(19) United States
(12) Patent Application Publication
(10) Pub. No.: US 2004/0102389 A1
(43) Pub. Date: May 27, 2004

(54) Nucleic acid-mediated treatment of diseases or conditions related to levels of vascular endothelial growth factor receptor (VEGF-R)

(75) Inventors: Pamela Pavco, Lafayette, CO (US); James McSwiggen, Boulder, CO (US); Dan Stinchcomb, Ft. Collins, CO (US); Jaime Escobedo, Alamo, CA (US); Julian Kim, Shaker Heights, OH (US); Daniel Lindner, Shaker Heights, OH (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Ribozyme Pharmaceuticals, Inc.

(21) Appl. No.: 10/287,949

(22) Filed: Nov. 4, 2002

Related U.S. Application Data
(63) Continuation-in-part of application No. 10/138,674, filed on May 3, 2002.
Application No. 10/138,674 is a continuation-in-part of application No. 09/870,161, filed on May 29, 2001.
Application No. 09/870,161 is a continuation-in-part of application No. 09/708,690, filed on Nov. 7, 2000.
Application No. 09/708,690 is a continuation-in-part of application No. 09/371,722, filed on Aug. 10, 1999, now Pat. No. 6,534,872.
Application No. 09/371,722 is a continuation-in-part of application No. 08/584,040, filed on Jan. 11, 1996, now Pat. No. 6,346,398.
(60) Provisional application No. 60/005,974, filed on Oct. 26, 1995.

(30) Foreign Application Priority Data
Oct. 25, 1996 (WO) PCT/US96/17480
May 29, 2002 (WO) PCT/US02/17674

(51) Int. Cl. A61K 48/00; C12N 5/08

(52) U.S. Cl. 514/44; 435/372; 435/375

(57) ABSTRACT

The present invention relates to nucleic acid molecules such as ribozymes, DNAzymes, short interfering RNA (siRNA), short interfering nuleic acid (siNA), and antisense which modulate the synthesis, expression and/or stability of an mRNA encoding one or more receptors of vascular endothelial growth factor, such as flt-1 (VEGFR1) and/or KDR (VEGFR2). Nucleic acid molecules and methods for the inhibition of angiogenesis and treatment of cancer and other conditions associated with VEGF-R are provided, optionally in conjunction with other therapeutic agents such as interferons.

This patent application is a continuation-in-part of Pavco et al., U.S. Ser. No. 10/138,674, filed May 3, 2002, entitled “Enzymatic Nucleic Acid-Mediated Treatment of Ocular Diseases or Conditions Related to Levels of Vascular Endothelial Growth Factor Receptor (VEGF-R)” which is a continuation-in-part of Pavco et al., U.S. Ser. No. 09/870,161, filed May 29, 2001, which is a continuation-in-part of Pavco et al., U.S. Ser. No. 09/708,690, filed Nov. 7, 2000, which is a continuation-in-part of Pavco et al., U.S. Ser. No. 09/371,722, filed Aug. 10, 1999, which is a continuation-in-part of Pavco et al., U.S. Ser. No. 08/584,040, filed Jan. 11, 1996, which claims the benefit of Pavco et al., U.S. S No. 60/005,974, filed on Oct. 26, 1995, all which are entitled “Method and Reagent for Treatment of Diseases or Conditions Related To Levels of Vascular Endothelial Growth Factor Receptor”. Each of these applications is hereby incorporated by reference herein in its entirety including the drawings and tables.

### BACKGROUND OF THE INVENTION

This invention relates to methods and reagents for the treatment of diseases or conditions relating to the levels of expression of vascular endothelial growth factor (VEGF) receptor(s).

The following is a discussion of relevant art, none of which is admitted to be prior art to the present invention.

VEGF, also referred to as vascular permeability factor (VPF) and vasculotropin, is a potent and highly specific mitogen of vascular endothelial cells (for a review see Ferrara, 1993 Trends Cardiovas. Med. 3, 244; Neufeld et al., 1994 Prog. Growth Factor Res. 5, 89). VEGF induced neovascularization is implicated in various pathological conditions such as tumor angiogenesis, proliferative diabetic retinopathy, hypoxia-induced angiogenesis, rheumatoid arthritis, psoriasis, wound healing and others.

VEGF, an endothelial cell-specific mitogen, is a 34-45 kDa glycoprotein with a wide range of activities that include promotion of angiogenesis, enhancement of vascular-permeability and others. VEGF belongs to the platelet-derived growth factor (PDGF) family of growth factors with approximately 18% homology with the A and B chain of PDGF at the amino acid level. Additionally, VEGF contains the eight conserved cysteine residues common to all growth factors belonging to the PDGF family (Neufeld et al., supra). VEGF protein is believed to exist predominantly as disulfide-linked homodimers; monomers of VEGF have been shown to be inactive (Plouet et al, 1989 EMBO J. 8, 3801).

VEGF exerts its influence on vascular endothelial cells by binding to specific high-affinity cell surface receptors. Covalent cross-linking experiments with ¹²⁵I-labeled VEGF protein have led to the identification of three high molecular weight complexes of 225, 195 and 175 kDa presumed to be VEGF and VEGF receptor complexes (Vaisman et al., 1990 J. Biol. Chem. 265, 19461). Based on these studies VEGF-specific receptors of 180, 150 and 130 kDa molecular mass were predicted. In endothelial cells, receptors of 150 and the 130 kDa have been identified. The VEGF receptors belong to the superfamily of receptor tyrosine kinases (RTKs) characterized by a conserved cytoplasmic catalytic kinase domain and a hydrophylic kinase sequence. The extracellular domains of the VEGF receptors consist of seven immunoglobulin-like domains that are thought to be involved in VEGF binding functions.

The two most abundant and high-affinity receptors of VEGF are flt-1 (fms-like tyrosine kinase) cloned by Shibuya et al., 1990 Oncogene 5, 519 and KDR (kinase-insert-domain-containing receptor) cloned by Terman et al., 1991 Oncogene 6, 1677. The murine homolog of KDR, cloned by Mathews et al., 1991, Proc. Natl. Acad. Sci., USA, 88, 9026, shares 85% amino acid homology with KDR and is termed as flk-1 (fetal liver kinase-1). Recently it has been shown that the high-affinity binding of VEGF to its receptors is modulated by cell surface-associated heparin and heparin-like molecules (Gitay-Goren et al., 1992 J. Biol. Chem. 267, 6093).

VEGF expression has been associated with several pathological states such as tumor angiogenesis, several forms of blindness, rheumatoid arthritis, psoriasis and others. Following is a brief summary of evidence supporting the involvement of VEGF in various diseases:

1) Tumor angiogenesis: Increased levels of VEGF gene expression have been reported in vascularized and edema-associated brain tumors (Berkman et al., 1993 J. Clini. Invest. 91, 153). A more direct demostration of the role of VEGF in tumor angiogenesis was demonstrated by Jim Kim et al., 1993 Nature 362,841 wherein, monoclonal antibodies against VEGF were successfully used to inhibit the growth of rhabdomyosarcoma, glioblastoma multiforme cells in nude mice. Similarly, expression of a dominant negative mutated form of the flt-1 VEGF receptor inhibits vascularization induced by human glioblastoma cells in nude mice (Millauer et al., 1994, Nature 367, 576).

2) Ocular diseases: Aiello et al., 1994 New Engl. J. Med. 331, 1480, showed that the ocular fluid of a majority of subjects suffering from diabetic retinopathy and other retinal disorders, contains a high concentration of VEGF. Miller et al., 1994 Am. J. Pathol. 145, 574, reported elevated levels of VEGF mRNA in subjects suffering from retinal ischemia. These observations support a direct role for VEGF in ocular diseases.

3) Psoriasis: Detmar et al., 1994 J. Exp. Med. 180, 1141 reported that VEGF and its receptors were over-expressed in psoriatic skin and psoriatic dermal microvessels, suggesting that VEGF plays a significant role in psoriasis.

4) Rheumatoid arthritis: Immunohistochemistry and in situ hybridization studies on tissues from the joints of subjects suffering from rheumatoid arthritis show an increased level of VEGF and its receptors (Fava et al., 1994 J. Exp. Med. 180, 341). Additionally, Koch et al., 1994 J. Immunol. 152, 4149, found that VEGF-specific antibodies were able to significantly reduce the mitogenic activity of synovial tissues from subjects suffering from rheumatoid arthritis. These 25 observations support a direct role for VEGF in rheumatoid arthritis.

5) Autosomal dominant polycystic kidney disease (ADPKD): ADPKD is the most common life threatening hereditary disease in the USA. It affects about 1:400 to 1:1000 people. Approximately 50% of people with ADPKD develop renal failure. ADPKD accounts for about 5-10% of end-stage renal failure in the United States requiring dialysis and renal transplantation. Several animal models, including the Han:SPRD rat model, mice with a targeted mutation in the Pkd2 gene, and congenital polycystic kidney (cpk) mice, closely resemble human ADPKD and present an opportunity to evaluate the therapeutic effect of agents that have the potential to interfere with one or more of the pathogenic elements of ADPKD. One of the features of ADPKD is angiogenesis. Angiogenesis may be necessary for growth of cyst cells as well as increased vascular permeability promoting fluid secretion into cysts. Proliferation of cystic epithelium is also a feature of ADPKD. Cyst cells in culture produce soluble vascular endothelial growth factor (VEGF), which is proven to be specific and critical for blood vessel formation. VEGF is also the best validated target for anti-angiogenesis therapies based on overwhelming genetic, mechanistic and animal efficacy data. However, VEGF can also directly stimulate proliferation of epithelial cells. VEGF triggers a response by interacting with cell-surface receptors. VEGFR1 has been detected in epithelial cells of cystic tubules but not in endothelial cells in the vasculature of cystic kidneys or normal kidneys. VEGFR2 expression is increased in endothelial cells of cyst vessels and in endothelial cells during renal ischemia-reperfusion. It is proposed that inhibition of VEGF receptors with anti-VEGFR1 and anti-VEGFR2 (KDR) agents (eg. nucleic acid molecules of the invention) would attenuate cyst formation, renal failure and mortality in ADPKD. Anti-VEGFR2 agents (eg. nucleic acid molecules of the invention) would inhibit angiogenesis involved in cyst formation. As VEGFR1 is present in cystic epithelium and not in vascular endothelium of cysts, it is proposed that anti-VEGFR1 agents would attenuate cystic epithelial cell proliferation and apoptosis which would in turn lead to less cyst formation. Further, it is proposed that VEGF produced by cystic epithelial cells is one of the stimuli for angiogenesis as well as epithelial cell proliferation and apoptosis. Validation assays for nucleic acid molecules of the invention can be performed in Han:SPRD rats, mice with a targeted mutation in the Pkd2 gene, and cpk mice. The effect of anti-VEGF nucleic acids on cyst formation and renal failure can determine the potential harmful role of angiogenesis in ADPKD.

In addition to the above data on pathological conditions involving excessive angiogenesis, a number of studies have demonstrated that VEGF is both necessary and sufficient for neovascularization. Takashita et al., 1995 J. Clin. Invest. 93, 662, demonstrated that a single injection of VEGF augmented collateral vessel development in a rabbit model of ischemia. VEGF also can induce neovascularization when injected into the cornea. Expression of the VEGF gene in CHO cells is sufficient to confer tumorigenic potential to the cells. Kim et al., supra and Millauer et al., supra used monoclonal antibodies against VEGF or a dominant negative form of flk-1 receptor to inhibit tumor-induced neovascularization.

During development, VEGF and its receptors are associated with regions of new vascular growth (Millauer et al., 1993 Cell 72, 835; Shalaby et al., 1993 J. Clin. Invest. 91, 2235). Furthermore, transgenic mice lacking either of the VEGF receptors are defective in blood vessel formation, in fact these mice do not survive; flk-1 appears to be required for differentiation of endothelial cells, while flt-1 appears to be required at later stages of vessel formation (Shalaby et al., 1995 Nature 376, 62; Fung et al., 1995 Nature 376, 66). Thus, these receptors must be present to properly signal endothelial cells or their precursors to respond to vascularization-promoting stimuli.

All of the conditions listed above, involve extensive vascularization. This hyper-stimulation of endothelial cells may be alleviated by VEGF antagonists. Thus most of the therapeutic efforts for the above conditions have concentrated on finding inhibitors of the VEGF protein.

Kim et al., 1993 Nature 362, 841 have been successful in inhibiting VEGF-induced tumor growth and angiogenesis in nude mice by treating the mice with VEGF-specific monoclonal antibody.

Koch et al., 1994 J. Immunol. 152, 4149 showed that the mitogenic activity of microvascular endothelial cells found in rheumatoid arthritis (RA) synovial tissue explants and the chemotactic property of endothelial cells from RA synovial fluid can be neutralized significantly by treatment with VEGF-specific antibodies.

Ullrich et al., International PCT Publication No. WO 94/11499 and Millauer et al., 1994 Nature 367, 576 used a soluble form of flk-1 receptor (dominant-negative mutant) to prevent VEGF-mediated tumor angiogenesis in immunodeficient mice.

Kendall and Thomas, International PCT Publication No. WO 94/21679 describe the use of naturally occuring or recombinantly-engineered soluble forms of VEGF receptors to inhibit VEGF activity.

Robinson, International PCT Publication No. WO 95/04142 describes the use of antisense oligonucleotides targeted against VEGF RNA to inhibit VEGF expression.

Jellinek et al., 1994 Biochemistry 33, 10450 describe the use of VEGF-specific high-affinity RNA aptamers to inhibit the binding of VEGF to its receptors.

Rockwell and Goldstein, International PCT Publication No. WO 95/21868, describe the use of anti-VEGF receptor monoclonal antibodies to neutralize the effect of VEGF on endothelial cells.

### SUMMARY OF THE INVENTION

The invention features novel nucleic acid-based compounds [e.g., enzymatic nucleic acid molecules (ribozymes such as Inozyme, G-cleaver, amberzyme, zinzyme), DNAzymes, antisense nucleic acids, 2-5A antisense chimeras, triplex forming nucleic acid, decoy nucleic acids, aptamers, allozymes, antisense nucleic acids containing RNA cleaving chemical groups (Cook et al., U.S. Pat. No. 5,359,051), small interfering RNA (siRNA), small interfering nucleic acid (siNA, Beigelman et al., U.S. S No. 60/409,293)] and methods for their use to down regulate or inhibit the expression of receptors of VEGF (VEGF-R such as VEGFR1 and/or VEGFR2).

In one embodiment, the invention features the use of one or more of the nucleic acid-based compounds to inhibit the expression of VEGFR1 (flt-1) and/or VEGFR2 (flk-1/KDR) receptors.

In another embodiment, the present invention features a compound having Formula I: (SEQ ID NO: 20818).

5′ gₛaₛgₛuₛugcUGAuGagg ccgaaa ggccGaaAgucugB 3′

wherein each a is 2′-O-methyl adenosine nucleotide, each g is a 2′-O-methyl guanosine nucleotide, each c is a 2′-O-methyl cytidine nucleotide, each u is a 2′-O-methyl uridine nucleotide, each A is adenosine, each G is guanosine, each s individually represents a phosphorothioate internucleotide linkage, U is 2′-deoxy-2′-C-allyl uridine, and B is an inverted deoxyabasic moiety.

In another embodiment, the present invention features a compound having Formula II: (SEQ ID NO: 13488).

5′-uₛaₛcₛaₛau ucU GAu Gag gcg aaa gcc Gaa Aag aca aB-3′

wherein each a is 2′-O-methyl adenosine nucleotide, each g is a 2′-O-methyl guanosine nucleotide, each c is a 2′-O-methyl cytidine nucleotide, each u is a 2′-O-methyl uridine nucleotide, each A is adenosine, each G is guanosine, each s individually represents a phosphorothioate internucleotide linkage, U is 2′-deoxy-2′-C-allyl uridine, and B is an inverted deoxyabasic moiety.

In one embodiment, the invention features a composition comprising a compound of Formula I and/or II in a pharmaceutically acceptable carrier or diluent.

In another embodiment, the invention features a method of administering to a cell, for example a mammalian cell or human cell, the compound of Formula I and/or II, comprising contacting the cell with the compound under conditions suitable for administration, for example in the presence of a delivery reagent. Examples of suitable delivery reagents include a lipid, cationic lipid, phospholipid, or liposome as described herein and known in the art.

In one embodiment, the invention features a method of administering to a cell the compound of Formula I or II in conjunction with a chemotherapeutic agent comprising contacting the cell with the compound and the chemotherapeutic agent under conditions suitable for administration.

Examples of chemotherapeutic agents that can be combined with the compound of Formula I and/or II include but are not limited to 5-fluoro uridine, Leucovorin, Irinotecan (CAMPTOSAR® or CPT-11 or Camptothecin-11 or Campto), Paclitaxel, or Carboplatin or a combination thereof.

In another embodiment, the present invention also features a cell comprising the compound of Formula I and/or II, wherein the cell is a mammalian cell. For example, in one embodiment the mammalian cell is a human cell.

In one embodiment, the invention features a method of inhibiting angiogenesis, for example tumor angiogenesis, in a subject comprising the step of contacting the subject with the compound of Formula I and/or II under conditions suitable for said inhibition. In one embodiment, the subject is a mammal, for example, a human.

In another embodiment, the invention features a method of treatment of a subject having a condition associated with an increased level of VEGF receptor, for example, cancers such as breast cancer, lung cancer, colorectal cancer, renal cancer, pancreatic cancer, or melanoma; Autosomal dominant polycystic kidney disease (ADPKD); or ocular indications such as diabetic retinopathy, or age related macular degeneration, comprising contacting one or more cells of the subject with the compound of Formula I and/or II, under conditions suitable for the treatment. In one embodiment, the subject is a human.

In another embodiment, the invention features a method of treatment of a subject having an ocular condition associated with an increased level of a VEGF receptor, for example, diabetic retinopathy, or age related macular degeneration, comprising contacting one or more cells of the subject with a nucleic acid molecule, such as an enzymatic nucleic acid molecule, targeted against a VEGF receptor RNA, e.g., a molecule according to Formula I and/or II, under conditions suitable for the treatment. In one embodiment, the subject is a human.

In yet another embodiment, a method of treatment of the invention further comprises the use of one or more drug therapies under conditions suitable for the treatment.

In one embodiment, the present invention also features a method of cleaving RNA comprising a sequence of VEGFR1 (flt-1) comprising contacting the compound of Formula I with the RNA under conditions suitable for the cleavage of the RNA, for example, where the cleavage is carried out in the presence of a divalent cation such as Mg2+.

In another embodiment, the invention features a method of administering to a mammal, for example a human, the compound of Formula I and/or II comprising contacting the mammal with the compound under conditions suitable for the administration, for example, in the presence of a delivery reagent such as a lipid, cationic lipid, phospholipid, or liposome.

In yet another embodiment, the invention features a method of administering to a mammal the compound of Formula I and/or II in conjunction with a chemotherapeutic agent comprising contacting the mammal, for example a human, with the compound and the chemotherapeutic agent under conditions suitable for the administration.

In another embodiment, the invention features a composition comprising the nucleic acid molecule of the instant invention and a pharmaceutically acceptable carrier or diluent. In one embodiment, the invention features one or more nucleic acid-based molecules and methods that independently or in combination modulate the expression of gene(s) encoding vascular endothelial growth factor receptors. Specifically, the present invention features nucleic acid molecules that modulate the expression of VEGF (for example Genbank Accession No. NM_{—}003376), VEGFR1 receptor (for example Genbank Accession No. NM_{—}002019), and VEGFR2 receptor (for example Genbank Accession No. NM_{—}002253) that are useful in preventing, treating, controlling, and/or diagnosing diseases and conditions described herein.

By “inhibit” it is meant that the activity of VEGF-R or level of VEGF-R mRNAs or equivalent RNAs encoding VEGF-R is reduced below that observed in the absence of a nucleic acid molecule of the instant invention. In one embodiment, inhibition with enzymatic nucleic acid preferably is below that level observed in the presence of an enzymatically inactive or attenuated molecule that is able to bind to the same site on the mRNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition with siRNA or siNA nucleic acid molecules is preferably below that level observed in the presence of, for example, an oligonucleotide with scrambled sequence or with mismatches. In another embodiment, inhibition of a VEGF-R gene with the nucleic acid molecule of the instant invention is greater in the presence of the nucleic acid molecule than in its absence.

By “VEGF-R” as used herein in meant a vascular endothelial growth factor receptor, for example VEGFR1 (also referred to as flt-1) and/or VEGFR2 (also referred to as flk-1 or kdr).

By “enzymatic nucleic acid molecule” it is meant an RNA molecule which has complementarity in a substrate binding region to a specified gene target, and also has an enzymatic activity which is active to specifically cleave target RNA. That is, the enzymatic RNA molecule is able to intermolecularly cleave RNA and thereby inactivate a target RNA molecule. The complementary region(s) allows sufficient hybridization of the enzymatic RNA molecule to the target RNA and thus permit cleavage. One hundred percent complementarity is preferred, but complementarity as low as 50-75% can also be useful in this invention. The nucleic acids can be modified at the base, sugar, and/or phosphate groups. The term enzymatic nucleic acid is used interchangeably with phrases such as ribozymes, catalytic RNA, enzymatic RNA, enzymatic DNA, catalytic DNA, catalytic oligonucleotides, nucleozyme, DNAzyme, Zinzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “enzymatic portion” or “catalytic domain” is meant that portion/region of the enzymatic nucleic acid essential for cleavage of a nucleic acid substrate (for example see FIG. 1).

By “substrate binding arm” or “substrate binding domain” is meant that portion/region of a enzymatic nucleic acid which is complementary to (i.e., able to base-pair with) a portion of its substrate. Generally, such complementarity is 100%, but can be less if desired. For example, as few as 10 bases out of 14 can be base-paired. Such arms are shown generally in FIG. 1. That is, these arms contain sequences within an enzymatic nucleic acid are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides, and are of sufficient length to stably interact with the target RNA; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in Ludwig et al., International PCT Publication No. WO 98/58058 and U.S. patent application Ser. No. 08/878,640. Inozymes possess endonuclease activity to cleave nucleic acid substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave nucleic acid substrates having a cleavage triplet NCN/, where N is a nucleotide, C is cytidine, and “/” represents the cleavage site. “I” represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in Eckstein et al., U.S. Pat. No. 6,127,173. G-cleavers possess endonuclease activity to cleave nucleic acid substrates having a cleavage triplet NYN/, where N is a nucleotide, Y is uridine or cytidine and “/” represents the cleavage site. G-cleavers can be chemically modified.

By “amberzyme” motif or configuration is meant, an enzymatic nucleic acid molecule comprising a motif as is generally described in Beigelman et al., International PCT publication No. WO 99/55857 and U.S. patent application Ser. No. 09/476,387. Amberzymes possess endonuclease activity to cleave nucleic acid substrates having a cleavage triplet NG/N, where N is a nucleotide, G is guanosine, and “/” represents the cleavage site. Amberzymes can be chemically modified to increase nuclease stability through substitutions. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif or configuration is meant, an enzymatic nucleic acid molecule comprising a motif as is generally described in Beigelman et al., International PCT publication No. WO 99/55857 and U.S. patent application Ser. No. 09/918,728. Zinzymes possess endonuclease activity to cleave nucleic acid substrates having a cleavage triplet including but not limited to YG/Y, where Y is uridine or cytidine, and G is guanosine and “/” represents the cleavage site. Zinzymes can be chemically modified to increase nuclease stability through substitutions, including substituting 2′-O-methyl guanosine nucleotides for guanosine nucleotides. In addition, differing nucleotide and/or non-nucleotide linkers can be used to substitute the 5′-gaaa-2′ loop of the motif. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is generally reviewed in Usman et al., U.S. Pat. No. 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection, see Santoro et al., supra and as generally described in Joyce et al., U.S. Pat. No. 5,807,718. Additional DNAzyme motifs can be selected for using techniques similar to those described in these references, and hence, are within the scope of the present invention.

By “sufficient length” is meant a nucleic acid molecule long enough to provide the intended function under the expected condition. For example, a nucleic acid molecule of the invention needs to be of “sufficient length” to provide stable binding to a target site under the expected binding conditions and environment. In another non-limiting example, for the binding arms of an enzymatic nucleic acid, “sufficient length” means that the binding arm sequence is long enough to provide stable binding to a target site under the expected reaction conditions and environment. The binding arms are not so long as to prevent useful turnover of the nucleic acid molecule.

By “stably interact” is meant interaction of the oligonucleotides with target, such as a target protein or target nucleic acid (e.g., by forming hydrogen bonds with complementary amino acids or nucleotides in the target under physiological conditions) that is sufficient for the intended purpose (e.g., specific binding to a protein target to disrupt the function of that protein or cleavage of target RNA/DNA by an enzyme).

By “equivalent” RNA to VEGF-R is meant to include those naturally occurring RNA molecules having homology (partial or complete) to VEGF-R, or encoding for proteins with similar function as VEGF-R in various animals, including human, rodent, primate, rabbit and pig. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target nucleic acid by means of RNA-RNA or RNA-DNA or RNA-PNA (protein nucleic acid; Egholm et al., 1993 Nature 365, 566) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, an antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target nucleic acid by means of DNA-RNA interactions, thereby activating RNase H, which digests the target nucleic acid in the duplex. The antisense oligonucleotides can comprise one or more RNAse H activating region, which is capable of activating RNAse H cleavage of a target nucleic acid. Antisense DNA can be synthesized chemically or expressed via the use of a single stranded DNA expression vector or equivalent thereof.

By “RNase H activating region” is meant a region (generally about 4-25 nucleotides in length, preferably about 5-11 nucleotides in length) of a nucleic acid molecule capable of binding to a target nucleic acid to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to a nucleic acid molecule-target nucleic acid complex and cleaves the target nucleic acid sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target nucleic acid in a sequence-specific manner and activate a cellular 2-5A-dependent ribonuclease which, in turn, cleaves the target nucleic acid (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded polynucleotide, such as DNA, in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

The term “complementarity” as used herein refers to the ability of a nucleic acid to form hydrogen bond(s) with another nucleic acid sequence by either traditional Watson-Crick or other non-traditional types. In reference to nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “nucleic acid decoy molecule”, or “decoy” as used herein is meant a nucleic acid molecule that mimics the natural binding domain for a ligand. The decoy therefore competes with the natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608).

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein. Similarly, the nucleic acid molecules of the instant invention can bind to VEGFR1 or VEGFR2 receptors to block activity of the receptor. This is a non-limiting example and those in the art will recognize that other embodiments can be readily generated using techniques generally known in the art, see for example Gold et al., U.S. Pat. No. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid moleule” as used herein refers to any nucleic acid molecule capable of mediating RNA interference “RNAi” or gene silencing; see for example Beigelman et al., U.S. S No. 60/409,293; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. For example the siNA can be a double-stranded polynucleotide molecule comprising self-complementary sense and antisense regions, wherein the antisense region comprises complementarity to a target nucleic acid molecule. The siNA can be a single-stranded hairpin polynucleotide having self-complementary sense and antisense regions, wherein the antisense region comprises complementarity to a target nucleic acid molecule. The siNA can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active siNA capable of mediating RNAi. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. In certain embodiments short interfering nucleic acids that do not require the presence of nucleotides having a 2′-hydroxy group for mediating RNAi are featured by the instant invention, and as such, short interfering nucleic acid molecules of the invention optionally do not contain any ribonucleotides (e.g., nucleotides having a 2′-OH group). The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA, short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transciptional gene silencing.

By “nucleic acid sensor molecule” or “allozyme” as used herein is meant a nucleic acid molecule comprising an enzymatic domain and a sensor domain, where the enzymatic nucleic acid domain's ability to catalyze a chemical reaction is dependent on the interaction with a target signaling molecule, such as a nucleic acid, polynucleotide, oligonucleotide, peptide, polypeptide, or protein, for example VEGF, VEGFR1 and/or VEGFR2. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520).

By “sensor component” or “sensor domain” of the nucleic acid sensor molecule as used herein is meant, a nucleic acid sequence (e.g., RNA or DNA or analogs thereof) which interacts with a target signaling molecule, for example a nucleic acid sequence in one or more regions of a target nucleic acid molecule or more than one target nucleic acid molecule, and which interaction causes the enzymatic nucleic acid component of the nucleic acid sensor molecule to either catalyze a reaction or stop catalyzing a reaction. In the presence of target signaling molecule of the invention, such as VEGF, VEGFR1 and/or VEGFR2, the ability of the sensor component, for example, to modulate the catalytic activity of the nucleic acid sensor molecule, is inhibited or diminished. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer) which is evolved to bind to a nucleic acid sequence within a target nucleic acid molecule (see for example Gold et al., U.S. Pat. No. 5,475,096 and 5,270,163). The sensor component can be covalently linked to the nucleic acid sensor molecule, or can be non-covalently associated. A person skilled in the art will recognize that all that is required is that the sensor component is able to selectively inhibit the activity of the nucleic acid sensor molecule to catalyze a reaction.

By “target molecule” or “target signaling molecule” is meant a molecule capable of interacting with a nucleic acid sensor molecule, specifically a sensor domain of a nucleic acid sensor molecule, in a manner that causes the nucleic acid sensor molecule to be active or inactive. The interaction of the signaling agent with a nucleic acid sensor molecule can result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, for example VEGF, VEGFR1 and/or VEGFR2.

The term “triplex forming oligonucleotides” as used herein refers to an oligonucleotide that can bind to a double-stranded DNA in a sequence-specific manner to form a triple-strand helix. Formation of such a triple helix structure has been shown to inhibit transcription of a targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

Seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA destroys its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

Nucleic acid molecules that target VEGF-R mRNA or specified sites in VEGF-R mRNAs represent a novel therapeutic approach to treat tumor angiogenesis, and cancers including, but not limited to, tumor and cancer types shown under Diagnosis in Table XX, ocular diseases, autosomal dominant polycystic kidney disease (ADPKD), rhuematoid arthritis, psoriasis and others. The nucleic acid molecules of the instant invention are able to inhibit the activity of VEGF-R (specifically flt-1 and flk-1/KDR). In another embodiment, the sequence specificity of siRNA and siNA or antisense acid molecules of the invention is required for their inhibitory effect. Those of ordinary skill in the art will find it clear from the exemplary nucleic acid molecules described that other nucleic acid molecules that cleave VEGF-R mRNAs or that otherwise mediate cleavage or inhibition of VEGF-R mRNA can be readily designed and are within the scope of the invention.

In one of the embodiments of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, Zinzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group TI introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071; of Zinzymes as is generally described by Beigelman et al., International PCT publication No. WO 99/55857 (see for example FIG. 32); and of DNAzymes as is generally described by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262 (see for example FIG. 33). NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

Enzymatic nucleic acid molecules of the invention that are allosterically regulated (“allozymes”) can be used to modulate VEGR receptor expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent. For example, allozymes can be designed to respond to signaling agents, such as flt-1 or kdr protein, flt-1 or kdr RNA, other proteins and/or RNAs involved in VEGF activity, and also, for example, compounds, metals, polymers, molecules and/or drugs that are targeted to VEGF or VEGF receptor, such as flt-1 or kdr expressing cells etc., which in turn modulate the activity of the enzymatic nucleic acid molecule. In response to interaction with a predetermined signaling agent, the activity of the allosteric enzymatic nucleic acid molecule is activated or inhibited such that the expression of a particular target is selectively down-regulated. The target can comprise flt-1 or kdr and/or a predetermined cellular component or receptor that modulates VEGF activity.

In a specific example, allosteric enzymatic nucleic acid molecules that are activated by interaction with a RNA encoding a flt-1 protein are used as therapeutic agents in vivo. The presence of RNA encoding the flt-1 protein activates the allosteric enzymatic nucleic acid molecule that subsequently cleaves the RNA encoding the flt-1 protein, resulting in the inhibition of flt-1 protein expression. In this manner, cells that express the flt-1 protein are selectively targeted.

In another non-limiting example, an allozyme can be activated by an flt-1 protein or peptide that caused the allozyme to inhibit the expression of flt-1 gene by, for example, cleaving RNA encoded by flt-1 gene. In this non-limiting example, the allozyme acts as a decoy to inhibit the function of flt-1 and also inhibit the expression of flt-1 once activated by the flt-1 protein.

In one embodiment, the nucleic acid molecule of the invention, e.g., antisense molecule, triplex DNA, or ribozyme, is 13 to 100 nucleotides in length, e.g., in specific embodiments 35, 36, 37, or 38 nucleotides in length (e.g., for particular ribozymes). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has a lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In another embodiment, a siRNA or siNA molecule of the invention comprises a duplex having two strands, one or both of which can be chemically modified, wherein each strand is between about 18 and about 27 (e.g., about 18, 19, 20, 21, 22, 23, 24, 25, 26, or 27) nucleotides in length, wherein the duplex has between about 18 and about 23 (e.g., about 18, 19, 20, 21, 22, or 23) base pairs. For example, an exemplary siNA or siRNA molecule of the invention comprises a duplex having two strands, one or both of which can be chemically modified (eg. sugar, base or backbone modification as described herein), wherein each strand consists of 21 nucleotides, each having 2 nucleotide 3′-overhangs, and wherein the duplex has 19 base pairs.

In another embodiment, a siNA or siRNA molecule of the invention comprises a single stranded hairpin structure, wherein the siNA is between about 36 and about 70 (e.g., about 36, 40, 45, 50, 55, 60, 65, or 70) nucleotides in length having between about 18 and about 23 (e.g., about 18, 19, 20, 21, 22, or 23) base pairs, and wherein the siNA or siRNA can include one or more chemical modifications described herein. For example, an siNA or siRNA molecule of the invention comprises a linear oligonucleotide having between about 42 and about 50 (e.g., about 42, 43, 44, 45, 46, 47, 48, 49, or 50) nucleotides that can be chemically modified, wherein the linear oligonucleotide forms a hairpin structure having 19 base pairs and a 2 nucleotide 3′-overhang.

In another embodiment, a linear hairpin siNA or siRNA molecule of the invention contains a stem loop motif, wherein the loop portion of the siNA or siRNA molecule is biodegradable. For example, a linear hairpin siNA molecule of the invention is designed such that degradation of the loop portion of the siNA or siRNA molecule in vivo can generate a double stranded siNA or siRNA molecule with 3′-overhangs, such as 3′-overhangs comprising about 2 nucleotides.

In one embodiment, the invention provides a method for producing nucleic acid molecules which exhibit a high degree of specificity for the RNA of a desired target. The nucleic acid molecule is preferably targeted to a highly conserved sequence region of target mRNAs encoding VEGF-R proteins (specifically flt-1 and/or flk-1/KDR) such that specific treatment of a disease or condition can be provided with either one or several enzymatic nucleic acids. Such enzymatic nucleic acid molecules can be delivered exogenously to specific tissue or cellular targets as required. Alternatively, the nucleic acid molecules can be expressed from DNA and/or RNA vectors that are delivered to specific cells.

By “highly conserved sequence region” is meant a nucleotide sequence of one or more regions in a nucleic acid molecule that does not vary significantly from one generation to the other or from one biological system to the other.

Synthesis of nucleic acids greater than 100 nucleotides in length is difficult using automated methods, and the therapeutic cost of such molecules is prohibitive. In this invention, small nucleic acid motifs (e.g., antisense oligonucleotides, siRNA, siNA, enzymatic nucleic acid molecules) are used for exogenous delivery. The simple structure of these molecules increases the ability of the nucleic acid to invade targeted regions of the mRNA structure. However, these nucleic acid molecules can also be expressed within cells from eukaryotic promoters (e.g., Izant and Weintraub, 1985 Science 229, 345; McGarry and Lindquist, 1986 Proc. Natl. Acad. Sci. USA 83, 399; SullengerScanlon et al., 1991, Proc. Natl. Acad. Sci. USA, 88, 10591-5; Kashani-Sabet et al, 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al., 1992 Nucleic Acids Res., 20, 4581-9; Sarver et al., 1990 Science 247, 1222-1225; Thompson et al., 1995 Nucleic Acids Res. 23, 2259). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO93/23569, and Sullivan et al., PCT WO94/02595, both hereby incorporated in their totality by reference herein; Ohkawa et al., 1992 Nucleic Acids Svmp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856).

The nucleic acid molecules of the invention are useful for the prevention of diseases and conditions related to the level of VEGF-R, including cancer (including but not limited to tumor and cancer types shown under Diagnosis in Table XX), diabetic retinopathy, macular degeneration, neovascular glaucoma, myopic degeneration, autosomal dominant polycystic kidney disease (ADPKD), arthritis, psoriasis, verruca vulgaris, angiofibroma of tuberous sclerosis, pot-wine stains, Sturge Weber syndrome, Kippel-Trenaunay-Weber syndrome, Osler-Weber-Rendu syndrome and any other diseases or conditions that are related to the levels of VEGF-R (specifically flt-1 and flk-1/KDR) in a cell or tissue.

By “diseases or conditions related to the level of VEGF-R” is meant that the reduction of VEGF-R (specifically flt-1 and flk-1/KDR) RNA levels and thus reduction in the level of the respective protein will relieve, to some extent, the symptoms of the disease or condition.

Nucleic acid molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers.

In one embodiment, the enzymatic nucleic acid molecule of the invention has one or more binding arms which are complementary to the substrate sequences in Tables II to IX, XIV-XIX, XXII, and XXIII. Examples of such enzymatic nucleic acid molecules also are shown in Tables II to IX, XIV-XIX, XXII, and XXIII. Examples of such enzymatic nucleic acid molecules consist essentially of sequences defined in these Tables.

In yet another embodiment, the invention features antisense nucleic acid molecules, siRNA, siNA, and/or 2-5A chimeras including sequences complementary to the target sequences shown in Tables II to IX, XIV-XIX, XXII, and XXIII. Such nucleic acid molecules can include sequences as shown for the binding arms of the enzymatic nucleic acid molecules in Tables II to IX, XIV-XIX, XXII, and XXIII. Similarly, triplex molecules can be provided targeted to the corresponding DNA target regions, and containing the DNA equivalent of a target sequence or a sequence complementary to the specified target (substrate) sequence. Typically, antisense and siRNA or siNA molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present that do not interfere with such cleavage. Thus, a core region can, for example, include one or more loop, stem-loop structure, or linker that does not prevent enzymatic activity. Thus, the underlined regions in the sequences in Tables II, IV, VI, VIII, XIV and XVI can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO: 20822), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

X can be a linker of >2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, X may be a non-nucleotide linker. In yet another embodiment, the nucleotide linker (X) can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press).

In yet another embodiment, the non-nucleotide linker (X) is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in one embodiment, the invention features an enzymatic nucleic acid molecule having one or more non-nucleotide moieties, and having enzymatic activity to cleave an RNA or DNA molecule.

In another aspect of the invention, nucleic acid molecules that cleave or inhibit expression of target RNA molecules and inhibit VEGF-R (specifically flt-1 and flk-1/KDR) activity are expressed from transcription units inserted into DNA or RNA vectors. The recombinant vectors are preferably DNA plasmids or viral vectors. Nucleic acid expressing viral vectors can be constructed based on, but not limited to, adeno-associated virus, retrovirus, adenovirus, or alphavirus vectors. Preferably, the recombinant vectors capable of expressing the nucleic acid molecules are delivered as described above, and persist in target cells. Alternatively, viral vectors can be used that provide for transient expression of nucleic acids. Such vectors can be repeatedly administered as necessary. Once expressed, the nucleic acids cleave, bind, and/or interact with the target mRNA. Delivery of nucleic acids expressing vectors can be systemic, such as by intravenous or intramuscular administration, by administration to target cells ex-planted from the subject followed by reintroduction into the subject, or by any other means that would allow for introduction into the desired target cell.

By “vectors” is meant any nucleic acid- and/or viral-based technique used to deliver a desired nucleic acid.

By “subject” is meant an organism which is a donor or recipient of explanted cells or the cells themselves. “Subject” also refers to an organism to which the nucleic acid molecules of the invention can be administered. In one embodiment, the subject is a mammal or mammalian cells. Preferably, the subject is a human or human cells.

The nucleic acid molecules of the instant invention, individually, or in combination or in conjunction with other drugs, can be used in the treatment of a disease or condition associated with VEGF-R, as discussed above. For example, a nucleic acid molecule of the invention can be administered individually or in combination with one or more drugs to a subject or the appropriate cells under conditions suitable for the treatment.

For example, to treat a disease or condition associated with VEGF-R levels, such as cancer (e.g., colorectal cancer, breast cancer), autosomal dominant polycystic kidney disease (ADPKD), or ocular diseases (e.g., diabetic retinopathy or age related macular degeneration) a subject may be treated, or other appropriate cells may be treated, as is evident to those skilled in the art, individually or in combination with one or more drugs under conditions suitable for the treatment.

In a further embodiment, the described molecules, such as siNA, siRNA, antisense or enzymatic nucleic acid molecules can be used in combination with other known treatments to treat conditions or diseases discussed above. For example, the described molecules can be used in combination with one or more known therapeutic agents to treat cancer and other diseases described herein.

In another embodiment, the invention features nucleic acid-based molecules [e.g., enzymatic nucleic acid molecules (ribozymes such as Inozyme, G-cleaver, amberzyme, zinzyme), DNAzymes, antisense nucleic acids, 2-5A antisense chimeras, triplex forming nucleic acid, decoy nucleic acids, aptamers, allozymes, antisense nucleic acids containing RNA cleaving chemical groups (Cook et al., U.S. Pat. No. 5,359,051), small interfering RNA (siRNA), small interfering nucleic acid (siNA, Beigelman et al., U.S. S No. 60/409,293)] and methods for their use to down regulate or inhibit the expression of genes capable of inducing angiogenesis (e.g., flt-1 and kdr).

In another embodiment, the invention features nucleic acid-based techniques [e.g., enzymatic nucleic acid molecules (ribozymes such as Inozyme, G-cleaver, amberzyme, zinzyme), DNAzymes, antisense nucleic acids, 2-5A antisense chimeras, triplex forming nucleic acid, decoy nucleic acids, aptamers, allozymes, antisense nucleic acids containing RNA cleaving chemical groups (Cook et al., U.S. Pat. No. 5,359,051), small interfering RNA (siRNA), small interfering nucleic acid (siNA, Beigelman et al., U.S. S No. 60/409,293)] and methods for their use to down regulate or inhibit the expression of VEGF receptor.

Other features and advantages of the invention will be apparent from the following description of the preferred embodiments thereof, and from the claims.

### BRIEF DESCRIPTION OF THE DRAWINGS

FIG. 1 is a diagrammatic representation of the hammerhead ribozyme domain known in the art. Stem II can be ≧2 base-pair long.

FIGS. 2a-d show hammerhead ribozyme substrate motifs. FIG. 2a is a diagrammatic representation of the hammerhead ribozyme domain known in the art; FIG. 2b is a diagrammatic representation of the hammerhead ribozyme as divided by Uhlenbeck (1987, Nature, 327, 596-600) into a substrate and enzyme portion; FIG. 2c is a similar diagram showing the hammerhead divided by Haseloff and Gerlach (1988, Nature, 334, 585-591) into two portions; and FIG. 2d is a similar diagram showing the hammerhead divided by Jeffries and Symons (1989, Nucl. Acids. Res., 17, 1371-1371) into two portions.

FIG. 3 is a diagrammatic representation of the general structure of a hairpin ribozyme.

Helix 2 (H2) is provided with at least 4 base pairs (i.e., n is 1, 2, 3 or 4) and helix 5 can be optionally provided of length 2 or more bases (preferably 3-20 bases, i.e., m is from 1-20 or more). Helix 2 and helix 5 can be covalently linked by one or more bases (i.e., r is ≧1 base). Helix 1, 4 or 5 can also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides can be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more can be modified chemically (abasic, base, sugar and/or phosphate modifications) or replaced with another base without significant effect. Helix 4 can be formed from two separate molecules, i.e., without a connecting loop. The connecting loop when present can be a ribonucleotide with or without modifications to its base, sugar or phosphate. “q” is ≧2 bases. The connecting loop can also be replaced with a non-nucleotide linker molecule. H refers to bases A, U, or C. Y refers to pyrimidine bases. “—” refers to a covalent bond.

FIG. 4 is a representation of the general structure of the hepatitis delta virus ribozyme domain known in the art.

FIG. 5 is a representation of the general structure of the VS RNA ribozyme domain.

FIG. 6 is a schematic representation of an RNAseH accessibility assay. Specifically, the left side of FIG. 6 is a diagram of complementary DNA oligonucleotides bound to accessible sites on the target RNA. Complementary DNA oligonucleotides are represented by broad lines labeled A, B, and C. Target RNA is represented by the thin, twisted line. The right side of FIG. 6 is a schematic of a gel separation of uncut target RNA from a cleaved target RNA. Detection of target RNA is by autoradiography of body-labeled, T7 transcript. The bands common to each lane represent uncleaved target RNA; the bands unique to each lane represent the cleaved products.

FIG. 7 shows the effect of hammerhead ribozymes targeted against flt-1 receptor on the binding of VEGF to the surface of human microvascular endothelial cells. Sequences of the ribozymes used are shown in Table II; the length of stem II region is 3 bp. The hammerhead ribozymes were chemically modified such that the ribozyme consists of ribose residues at five positions (see FIG. 11); U4 and U7 positions contain 2′-NH₂ modifications, the remaining nucleotide positions contain 2′-O-methyl substitutions; four nucleotides at the 5′ terminus contains phosphorothioate substitutions. Additionally, the 3′ end of the ribozyme contains a 3′-3′ linked inverted abasic deoxyribose. The results of two separate experiments are shown as separate bars for each set. Each bar represents the average of triplicate samples. The standard deviation is shown with error bars. For the flt-1 data, 500 nM ribozyme (3:1 charge ratio with LipofectAMINE®) was used. Control 1-10 is the control for ribozymes 307-2797, control 1-20 is the control for ribozymes 3008-5585. The Control 1-10 and Control 11-20 represent the treatment of cells with LipofectAMINE® alone without any ribozymes.

FIG. 8 shows the effect of hammerhead ribozymes targeted against KDR receptor on the binding of VEGF to KDR on the surface of human microvascular endothelial cells. Sequences of the ribozymes used are shown in Table IV; the length of stem II region is 3 bp. The hammerhead ribozymes were chemically modified such that the ribozyme consists of ribose residues at five positions (see FIG. 11); U4 and U7 positions contain 2′-NH₂ modifications, the remaining nucleotide positions contain 2′-O-methyl substitutions; four nucleotides at the 5′ terminus contains phosphorothioate substitutions. Additionally, the 3′ end of the ribozyme contains a 3′-3′ linked inverted abasic deoxyribose. The Control 1-10 and Control 11-20 represent the treatment of cells with LipofectAMINE® alone without any ribozymes. Irrel. RZ, is a control experiment wherein the cells are treated with, a non-KDR-targeted ribozyme complexed with Lipofectamine®. 200 nM ribozyme (3:1 charge ratio with LipofectAMINE®) was used. In addition to the KDR-targeted ribozymes, the effect on VEGF binding of a ribozyme targeted to an irrelevant mRNA (irrel. RZ) is also shown. Because the affinity of KDR for VEGF is about 10-fold lower than the affinity of flt-1 for VEGF, a higher concentration of VEGF was used in the binding assay.

FIG. 9 shows the specificity of hammerhead ribozymes targeted against flt-1 receptor. Inhibition of the binding of VEGF, urokinase plasminogen activator (UPA) and fibroblast growth factor (FGF) to their corresponding receptors as a function of anti-FLT ribozymes is shown. The sequence and description of the ribozymes used are as described in FIG. 7 above. The average of triplicate samples is given; percent inhibition as calculated below.

FIG. 10 shows the inhibition of the proliferation of Human aortic endothelial cells (HAEC) mediated by phosphorothioate antisense oligodeoxynucleotides targeted against human KDR receptor RNA. Cell proliferation (O.D. 490) as a function of antisense oligodeoxynucleotide concentration is shown. KDR 21 AS represents a 21 nt phosphorothioate antisense oligodeoxynucleotide targeted against KDR RNA. KDR 21 Scram represents a 21 nt phosphorothioate oligodeoxynucleotide having a scrambled sequence. LF represents the lipid carrier Lipofectin.

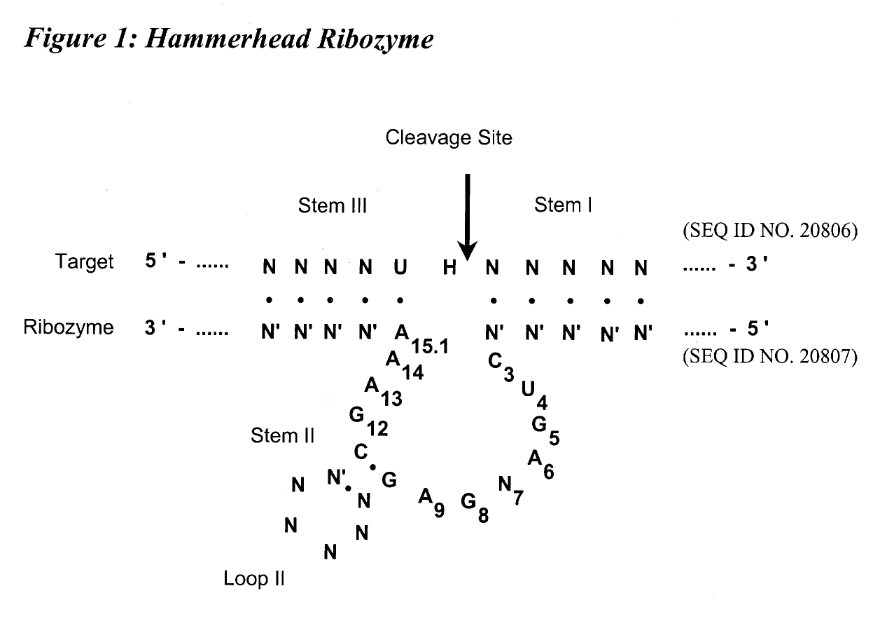

FIGS. 1A and B show a diagrammatic representation of hammerhead ribozymes targeted against flt-1 RNA and in vitro cleavage of flt-1 RNA by hammerhead ribozymes. The hammerhead (HH) ribozymes were chemically modified such that the ribozyme consists of ribose residues at five positions; U4 and U7 positions contain 2′-NH₂ modifications, the remaining nucleotide positions contain 2′-O-methyl substitutions; four nucleotides at the 5′ terminus contains phosphorothioate substitutions. Additionally, the 3′ end of the ribozyme contains a 3′-3′ linked inverted abasic deoxyribose (designated as 3′-1H). FIG. 11A shows hammerhead ribozymes 1358 HH-A and 4229 HH-A, which contain a 3 base-paired stem It region. FIG. 11B shows hammerhead ribozymes 1358 HH-B and 4229 HH-B, which contain a 4 base-paired stem II region. FIGS. 11C and 11D show in vitro cleavage kinetics of hammerhead ribozymes targeted against sites 1358 and 4229 within the flt-1 RNA

FIG. 12 shows a diagrammatic representation of hammerhead (HH) ribozymes targeted against sites 1358 and 4229 within the flt-1 RNA. The hammerhead (HH) ribozymes were chemically modified such that the ribozyme consists of ribose residues at five positions; U4 position contains 2′-C-allyl modification, the remaining nucleotide positions contain 2′-O-methyl substitutions; four nucleotides at the 5′ terminus contains phosphorothioate substitutions.

Additionally, the 3′ end of the ribozyme contains a 3′-3′ linked inverted abasic deoxyribose (designated as 3′-1H). FIG. 12B shows a graphical representation of the inhibition of cell proliferation mediated by 1358HH and 4229HH ribozymes

FIG. 13 shows inhibition of human microvascular endothelial cell proliferation mediated by anti-KDR hammerhead ribozymes. The figure is a graphical representation of the inhibition of cell proliferation mediated by hammerhead ribozymes targeted against sites 527, 730, 3702 and 3950 within the KDR RNA. Irrelevant HH RZ is a hammerhead ribozyme targeted to an irrelevant target. All of these ribozymes, including the Irrelevant HH RZ, were chemically modified such that the ribozyme consists of ribose residues at five positions; U4 and U7 positions contain 2′-NH₂ modifications, the remaining nucleotide positions contain 2′-O-methyl substitutions; four nucleotides at the 5′ termini contain phosphorothioate substitutions. Additionally, the 3′ end of the ribozyme contains a 3′-3′ linked inverted abasic deoxyribose (3′-1H).

FIG. 14 shows in vitro cleavage of KDR RNA by hammerhead ribozymes. The hammerhead (HH) ribozymes were chemically modified such that the ribozyme consists of ribose residues at five positions; U4 and U7 positions contain 2′-NH₂ modifications, the remaining nucleotide positions contain 2′-O-methyl substitutions. Additionally, the 3′ end of the ribozyme contains a 3′-3′ linked inverted abasic deoxyribose (designated as 3′-1H). 726 HH and 527 HH contain 4 base-paired stem II region. Percent in vitro cleavage kinetics as a function of time of HH ribozymes targeted against sites 527 and 726 within the KDR RNA is shown.

FIG. 15 shows in vitro cleavage of KDR RNA by hammerhead ribozymes. The hammerhead (HH) ribozymes were chemically modified such that the ribozyme consists of ribose residues at five positions; U4 and U7 positions contain 2′-NH₂ modifications, the remaining nucleotide positions contain 2′-O-methyl substitutions. Additionally, the 3′ end of the ribozyme contains a 3′-3′ linked inverted abasic deoxyribose (designated as 3′-1H). 3702 HH and 3950 HH contain 4 base-paired stem II region. Percent in vitro cleavage kinetics as a function of time of HH ribozymes targeted against sites 3702 and 3950 within the KDR RNA is shown.

FIG. 16 shows in vitro cleavage of RNA by hammerhead ribozymes that are targeted to sites that are conserved between flt-1 and KDR RNA. The hammerhead (HH) ribozymes were chemically modified such that the ribozyme consists of ribose residues at five positions; U4 and U7 positions contain 2′-NH₂ modifications, the remaining nucleotide positions contain 2′-O-methyl substitutions. Additionally, the 3′ end of the ribozyme contains a 3′-3′ linked inverted abasic deoxyribose (designated as 3′-1H). FLT/KDR-1HH ribozyme was synthesized with either a 4 base-paired or a 3 base-paired stem II region. FLT/KDR-1HH can cleave site 3388 within flt-1 RNA and site 3151 within KDR RNA. Percent in vitro cleavage kinetics as a function of time of HH ribozymes targeted against sites 3702 and 3950 within the KDR RNA is shown.

FIG. 17 shows inhibition of human microvascular endothelial cell proliferation mediated by anti-KDR and anti-flt-1 hammerhead ribozymes. The figure is a graphical representation of the inhibition of cell proliferation mediated by hammerhead ribozymes targeted against sites KDR sites-527, 726 or 3950 or flt-1 site 4229. The figure also shows enhanced inhibition of cell proliferation by a combination of flt-1 and KDR hammerhead ribozymes. 4229+527, indicates the treatment of cells with both the flt 4229 and the KDR 527 ribozymes. 4229+726, indicates the treatment of cells with both the flt 4229 and the KDR 726 ribozymes. 4229+3950, indicates the treatment of cells with both the flt 4229 and the KDR 3950 ribozymes. VEGF-, indicates the basal level of cell proliferation in the absence of VEGF. A, indicates catalytically active ribozyme; I, indicates catalytically inactive ribozyme. All of these ribozymes were chemically modified such that the ribozyme consists of ribose residues at five positions; U4 and U7 positions contain 2′-NH₂ modifications, the remaining nucleotide positions contain 2′-O-methyl substitutions; four nucleotides at the 5′ termini contain phosphorothioate substitutions. Additionally, the 3′ end of the ribozyme contains a 3′-3′ linked inverted abasic deoxyribose (3′-1H).

FIG. 18 shows the inhibition of VEGF-induced angiogenesis in rat cornea mediated by anti-flt-1 hammerhead ribozyme. All of these ribozymes were chemically modified such that the ribozyme consists of ribose residues at five positions; U4 position contains 2′-C-allyl modifications, the remaining nucleotide positions contain 2′-O-methyl substitutions; four nucleotides at the 5′ termini contain phosphorothioate substitutions. Additionally, the 3′ end of the ribozyme contains a 3′-3′ linked inverted abasic deoxyribose (3′-1H). A decrease in the Surface Area corresponds to a reduction in angiogenesis. VEGF alone corresponds to treatment of the cornea with VEGF and no ribozymes. Vehicle alone corresponds to the treatment of the cornea with the carrier alone and no VEGF. This control gives a basal level of Surface Area. Active 4229 HH, corresponds to the treatment of cornea with the flt-1 4229 HH ribozyme in the absence of any VEGF. This control also gives a basal level of Surface Area. Active 4229 HH+VEGF, corresponds to the co-treatment of cornea with the flt-1 4229 HH ribozyme and VEGF. Inactive 4229 HH+VEGF, corresponds to the co-treatment of cornea with a catalytically inactive version of 4229 HH ribozyme and VEGF.

FIG. 19 shows ribozyme-mediated inhibition of cell proliferation. Cultured HMVEC-d were treated with ribozyme or attenuated controls as LIPOFECTAMINE™ complexes. After treatment, cells were stimulated with VEGF₁₆₅ or bFGF and allowed to grow for 48 h prior to determining the cell number. Each ribozyme was tested in triplicate at three concentrations and data are presented as mean cell number per well +SD. The data obtained following ribozyme treatment and VEGF stimulation are presented in panels A & B for anti-Flt-1 ribozymes and panels D & E for anti-KDR ribozymes. Representative data obtained following ribozyme treatment and bFGF stimulation are shown in panel C for one anti-Flt-1 ribozyme and in panel F for one anti-KDR ribozyme. In all panels, active ribozymes are represented with filled symbols; attenuated controls with open symbols. In addition to the ribozymes and attenuated controls listed in Table XII, a second set having the same sequences but with an additional basepair in the “stem II” region of the ribozyme are also shown for VEGF-induced proliferation studies. These 4 bp stem II ribozymes and attenuated controls have one additional base pair such that the stem II/loop sequence is ggccgaaaggcc. Therefore, ribozymes and controls with 3 or 4 basepair stem IIs are denoted with circles and squares, respectively. The data for one irrelevant ribozyme (filled triangle, panel B) are also shown. This irrelevant ribozyme contains an active core sequence but has no binding site in either Flt-1 or KDR mRNA. Its sequence is 5′-gₛaₛaₛgₛgaacUGAuGaggccgaaaggccGaaAgauggcT-3′ with modifications as in Table XII except that T indicates a 3′-3′ inverted deoxythymidine. For reference, the average number of cells in control wells after 48 h in the absence of VEGF or bFGF for each of the panels are as follows: A, B, C, 12477+617; D, E, F, 17182+1053.

FIG. 20 shows target specificity of anti-Flt-1 and KDR ribozymes. Cultured HMVEC-d were treated with LIPOFECTAMINEE™ complexes containing 200 nM active ribozyme (A) or attenuated control (C) and analyzed by RNAse protection following 24 h of VEGF-stimulated growth. Data obtained for ribozymes and attenuated controls that target Flt-1 site 4229 or KDR site 726 are shown. Data were normalized to the level of an internal mRNA control (cyclophilin) and are presented as percent decrease in Flt-1 (left panel) or KDR mRNA (right panel) relative to an untreated control. Error bars indicate the range of duplicate samples.

FIG. 21 shows antiangiogenic efficacy of ribozyme in the rat corneal model of VEGF-induced angiogenesis. The percent inhibition of VEGF-induced angiogenesis for locally administered anti-Flt-1 (site 4229) ribozyme (filled circles) and their attenuated controls (open circles) are plotted over the dose range tested. Pixels associated with background structures including the iris were subtracted from all treatment groups. Data are expressed as mean percent reduction in VEGF-induced angiogenesis ±SEM. * p<0.05 relative to VEGF/vehicle treated controls by Dunnett's, **p<0.05 relative to attenuated dose-matched controls by Tukey-Kramer.

FIG. 22 shows antiangiogenic efficacy of ribozyme in the rat corneal model of VEGF-induced angiogenesis. The percent inhibition of VEGF-induced angiogenesis for locally administered anti-KDR (site 726) ribozyme (filled circles) and their attenuated controls (open circles) are plotted over the dose range tested. Pixels associated with background structures including the iris were subtracted from all treatment groups. Data are expressed as mean percent reduction in VEGF-induced angiogenesis ±SEM. * p<0.05 relative to VEGF/vehicle treated controls by Dunnett's, **p<0.05 relative to attenuated dose-matched controls by Tukey-Kramer.

FIG. 23 shows the effect of subcutaneous bolus administration of ANGIOZYME™ in a mouse Lewis Lung Carcinoma (LLC) model.

FIG. 24 shows the effect of ANGIOZYME™ in combination with gemcitabine or cyclophosphamide on primary tumor growth in the mouse LLC model.

FIG. 25 shows the effect of ANGIOZYME™ in combination with gemcitabine or cyclophosphamide on tumor metastases in the mouse LLC model.

FIG. 26 shows a secondary structure model of ANGIOZYME™ ribozyme bound to its RNA target.

FIG. 27 shows a time course of inhibition of primary tumor growth following systemic administration of ANGIOZYME™ in the LLC mouse model.

FIG. 28 shows inhibition of primary tumor growth following systemic administration of ANGIOZYME™ according to a certain dosing regimen in the LLC mouse model.

FIG. 29 shows a dose-dependent inhibition of tumor metastases following systemic administration of ANGIOZYME™ in a mouse colorectal model.

FIG. 30 shows inhibition of liver metastases following systemic administration of ANGIOZYME™ in a mouse colorectal model.

FIG. 31 is a graph showing the plasma concentration profile of ANGIOZYME™ after a single subcutaneous (SC) dose of 10, 30, 100 or 300 mg/m².

FIG. 32 shows an example of the Zinzyme enzymatic nucleic acid motif that is chemically stabilized (see for example Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

FIG. 33 shows an example of a DNAzyme motif described generally, for example in Santoro et al., 1997, PNAS, 94, 4262.

FIGS. 34A and B show a mouse model protocol and results of proliferative retinopathy.

FIG. 34A shows anoutline for the mouse model of proliferative retinopathy showing the points of ribozyme administration. FIG. 34B shows a graph demonstrating the efficacy of a VEGF-receptor-targeted enzymatic nucleic acid molecule in a mouse model of proliferative retinopathy.

FIG. 35 shows the effect of anti-VEGFR1 and VEGFR enzymatic nucleic acid molecules on primary subcutaneous tumor growth. Tumor size (in mm2) was measured following treatment with anti-VEGFR1, anti-VEGFR2, or both anti-VEGFR1 and anti-VEGFR2 enzymatic nucleic acid molecules compared to a control (HBSS). As shown in the Figure, significant inhibition of tumor growth resuts from the combination treatment with anti-VEGFR1 and anti-VEGFR2 enzymatic nucleic acid molecules.

### DETAILED DESCRIPTION OF THE INVENTION

Mechanism of action of Nucleic Acid Molecules of the Invention

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which acts as substrates for RNase H are phosphorothioates and phosphorodithioates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. S No. 60/082,404 which was filed on Apr. 20, 1998; Hartmann et al., U.S. S No. 60/101,174 which was filed on Sep. 21, 1998) all of these are incorporated by reference herein in their entirety.

Triplex Forming Oligonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra)

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Enzymatic nucleic acid molecules of this invention block to some extent VEGF-R (specifically flt-1 and flk-1/KDR) production and can be used to treat disease or diagnose such disease. Enzymatic nucleic acid molecules are delivered to cells in culture, to cells or tissues in animal models of angiogenesis and/or RA and to human cells or tissues ex vivo or in vivo.

Enzymatic nucleic acid molecule cleavage of VEGF-R RNAs (specifically RNAs that encode flt-1 and flk-1/KDR) in these systems can alleviate disease symptoms.

The enzymatic nature of enzymatic nucleic acid molecules, such as ribozymes, has significant advantages, such as the concentration of enzymatic nucleic acid necessary to affect a therapeutic treatment is lower. This advantage reflects the ability of the enucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, enzymatic nucleic acids, such as trans-cleaving ribozymes can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Short Interfering Nucleic Acid (siRNA/siNA): The discussion that follows discusses the proposed mechanism of RNA interference mediated by short interfering RNA as is presently known, and is not meant to be limiting and is not an admission of prior art. Applicant has determined that chemically modified short interfering nucleic acids possess similar or improved capacity to mediate RNAi as do siRNA molecules and are expected to possess improved stability and activity in vivo; therefore, this discussion is not meant to be limiting only to siRNA and can be applied to siNA as a whole. By “improved capacity to mediate RNAi” is meant to include RNAi activity measured in vitro and/or in vivo where the RNAi activity is a reflection of both the ability of the siNA to mediate RNAi and the stability of the siRNAs of the invention. In this invention, the product of these activities can be increased in vitro and/or in vivo compared to an all RNA siRNA or an siNA containing a plurality of ribonucleotides. In some cases, the activity or stability of the siNA molecule can be decreased (i.e., less than ten-fold), but the overall activity of the siNA molecule is enhanced, in vitro and/or in vivo.

RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease m enzyme referred to as Dicer. Dicer is involved in the processing of the dsRNA into short pieces of dsRNA known as short interfering RNAs (siRNA) (Berstein et al., 2001, Nature, 409, 363). Short interfering RNAs derived from Dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15,188).

RNAi has been studied in a variety of systems. Fire et al., 1998, Nature, 391, 806, were the first to observe RNAi in C. elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications which concern inactivation of expression of disease-related genes: WO 95/23225, WO 95/13380, WO 94/02595, incorporated by reference herein. Target sites useful for siRNA/siNA molecules of the invention can be determined as disclosed in, for example, Beigelman et al., U.S. S No. 60/409,293. Rather than repeat the guidance provided in those documents here, below are provided specific examples of such methods, not limiting to those with skill in the art. Nucleic acid molecules to such targets are designed as described in those applications and synthesized to be tested in vitro and in vivo, as also described.

In a non-limiting example, the sequence of human and mouse flt-1, KDR and/or flk-1 mRNAs were screened for optimal enzymatic nucleic acid target sites using a computer folding algorithm. Hammerhead, hairpin, NCH, or G-Cleaver ribozyme cleavage sites were identified. These sites are shown in Tables II to IX, XIV-XIX, XXII, and XXIII (all sequences are 5′ to 3′ in the tables; X can be any base-paired sequence, the actual sequence is not relevant here). The nucleotide base position is noted in the Tables as that site to be cleaved by the designated type of ribozyme. While mouse and human sequences can be screened and enzymatic nucleic acid molecules thereafter designed, the human targeted sequences are of most utility. However, as discussed in Stinchcomb et al., WO 95/23225, mouse targeted enzymatic nucleic acid can be useful to test efficacy of action of the enzymatic nucleic acid molecule prior to testing in humans. The nucleotide base position is noted in the Tables as that site to be cleaved by the designated type of enzymatic nucleic acid. Similarly, antisense nucleic acid molecules, siRNA, and/or siNA molecules of the invention can be desinged to target sequences shown in the Tables herein or sequences derived from Accession Numbers herein.

Enzymatic nucleic acid molecules are designed that bind and cleave target RNA in a sequence-specific manner, whereas antisense and siRNA/siNA molecules are desinged to be complementary to the target sequences. The nucleic acid molecules can be individually analyzed by computer folding (Jaeger et al, 1989 Proc. Natl. Acad. Sci. USA, 86, 7706) to assess whether the nucleic acid sequences fold into the appropriate secondary structure or if secondary structure will interfere with acitivity (eg. siRNA/siNA and antisense sequences). Those nucleic acid molecules with unfavorable intramolecular interactions are eliminated from consideration. Varying binding arm lengths or overall sequence lengths can be chosen to optimize activity.

In a non-limiting example, referring to FIG. 6, mRNA was screened for accessible cleavage sites by the method described generally in Draper et al, PCT WO93/23569, hereby incorporated by reference herein. Briefly, DNA oligonucleotides complementary to potential hammerhead or hairpin ribozyme cleavage sites were synthesized. A polymerase chain reaction was used to generate substrates for T7 RNA polymerase transcription from human and mouse flt-1, KDR and/or flk-1 cDNA clones. Labeled RNA transcripts were synthesized in vitro from the templates. The oligonucleotides and the labeled transcripts were annealed, RNAseH was added and the mixtures were incubated for the designated times at 37° C. Reactions were stopped and RNA separated on sequencing polyacrylamide gels. The percentage of the substrate cleaved was determined by autoradiographic quantitation using a PhosphorImaging system. From these data, antisense oligonucleotides, and ribozymes, such as hammerhead or hairpin ribozyme sites are chosen as the most accessible.

Ribozymes of the hammerhead or hairpin motif were designed to anneal to various sites in the mRNA message. The binding arms are complementary to the target site sequences described above. The ribozymes were chemically synthesized. The method of synthesis used follows the procedure for normal RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., siRNA/siNA, antisense oligonucleotides, enzymatic nucleic acid molecules) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211,3-19, and is incorporated herein by reference.

The method of synthesis used for normal RNA including certain nucleic acid molecules of the invention follows the procedure as described in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684 and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 μmol scale protocol with a 7.75 min coupling step for alkylsilyl protected nucleotides and a 2.5 min coupling step for 2′-O-methylated nucleotides. Table XI outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 15-fold excess (31 μL of 0.1 M=3.1 μmol) of phosphoramidite and a 38.7-fold excess of S-ethyl tetrazole (31 μL of 0.25 M=7.75 μmmol) relative to polymer-bound 5′-hydroxyl was used in each coupling cycle. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems, Inc. synthesizer; detritylation solution was 3% TCA in methylene chloride (ABI); capping was performed with 16% N-methyl imidazole in THF (ABI) and 10% acetic anhydride/10% 2,6-lutidine in THF (ABI); oxidation solution was 16.9 mM I₂, 49 mM pyridine, 9% water in THF (PERSEPTIVE™). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H2O/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO:1/1 (0.8 mL) at 65° C. for 15 min. The vial was brought to r.t. TEA.3HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO₃ solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCl and washed with water again. The oligonucleotide was then eluted with 30% acetonitrile.

Inactive hammerhead ribozymes or binding attenuated control (BAC) oligonucleotides) were synthesized by substituting a U for G5 and a U for A14 (numbering from Hertel, K. J., et al., 1992, Nucleic Acids Res., 20, 3252).

The average stepwise coupling yields were >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together by ligation (Moore et al., 1992, Science 256, 9923; Draper et al., International PCT publication No. WO 93/23569; Shabarova et al., 1991, Nucleic Acids Research 19, 4247)

Nucleic acid molecules are modified extensively to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34; Usman et al., 1994 Nucleic Acids Symp. Ser. 31, 163). Nucleic acid molecules are purified by gel electrophoresis using general methods or are purified by high pressure liquid chromatography (HPLC; See Wincott et al., Supra, the totality of which is hereby incorporated herein by reference) and are resuspended in water.

For example, the sequences of the enzymatic nucleic acid molecules that are chemically synthesized, useful in this study, are shown in Tables II to IX, XIV-XIX, XXII, and XXIII.

Those in the art will recognize that these sequences are representative only of many more such sequences where the enzymatic portion of the enzymatic nucleic acid (all but the binding arms) is altered to affect activity. Stem-loop IV sequence of hairpin ribozymes listed in, for example, Table III (5′-CACGUUGUG-3′) can be altered (substitution, deletion, and/or insertion) to contain any sequence, provided a minimum of two base-paired stem structure can form. Preferably, no more than 200 bases are inserted at these locations. The sequences listed in Tables II to X, XII-XIX, XXII, and XXIII may be formed of ribonucleotides or other nucleotides or non-nucleotides. Such molecules with enzymatic activity are equivalent to the enzymatic nucleic acids described specifically in the Tables.

Optimizing Activity of the Nucleic Acid Molecule of the Invention.

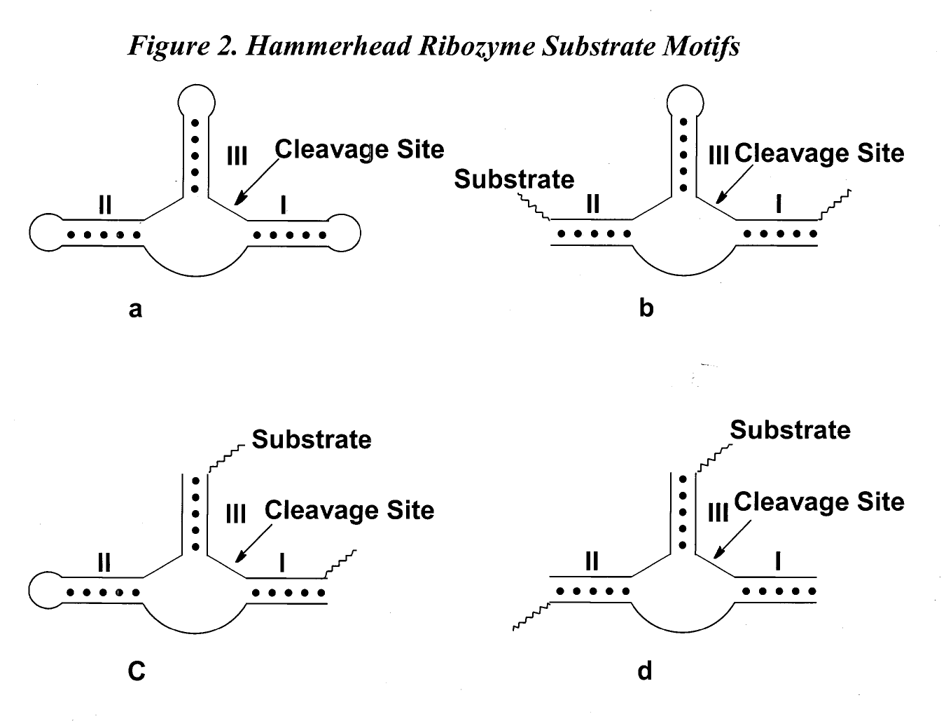

Nucleic acid activity can be optimized as described by Stinchcomb et al., supra. The details will not be repeated here, but include altering the length of the nucleic acid binding arms (stems I and III, see FIG. 2c), or chemically synthesizing enzymatic nucleic acid with modifications that prevent their degradation by serum ribonucleases (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al, International Publication No. WO 93/15187; Rossi et al., International Publication No. WO 91/03162; Beigelman et al., 1995 J. Biol. Chem. in press; as well as Sproat, U.S. Pat. No. 5,334,711 which describe various chemical modifications that can be made to the sugar moieties of enzymatic RNA molecules). Modifications which enhance their efficacy in cells, and removal of stem II bases to shorten RNA synthesis times and reduce chemical requirements are desired. (All these publications are hereby incorporated by reference herein).

There are several examples in the art describing sugar, base and phosphate modifications that can be introduced into enzymatic nucleic acid molecules without significantly effecting catalysis and with significant enhancement in their nuclease stability and efficacy. For example, enzymatic nucleic acid molecules are modified to enhance stability and/or enhance catalytic activity by modification with nuclease resistant groups, for example, 2′-amino, 2′-C-allyl, 2′-fluoro, 2′-O-methyl, 2′-H, nucleotide base modifications (for a review see Usman and Cedergren, 1992 TIBS 17, 34; Usman et al., 1994 Nucleic Acids Symp. Ser. 31, 163; Burgin et al., 1996 Biochemistry 35, 14090). Sugar modification of enzymatic nucleic acid molecules have been extensively described in the art (see Eckstein et al., International Publication PCT No. WO 92/07065; Perrault et al. Nature 1990, 344, 565-568; Pieken et al. Science 1991, 253, 314-317; Usman and Cedergren, Trends in Biochem. Sci. 1992, 17, 334-339; Usman et al. International Publication PCT No. WO 93/15187; Sproat, U.S. Pat. No. 5,334,711 and Beigelman et al., 1995 J. Biol. Chem. 270, 25702; all of the references are hereby incorporated in their totality by reference herein). Such publications describe general methods and strategies to determine the location of incorporation of sugar, base and/or phosphate modifications and the like into enzymatic nucleic acid without inhibiting catalysis, and are incorporated by reference herein. In view of such teachings, similar modifications can be used as described herein to modify the nucleic acid catalysts of the instant invention.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. As exemplified herein such enzymatic nucleic acid molecules are useful in a cell and/or in vivo even if activity over all is reduced 10 fold (Burgin et al., 1996, Biochemistry, 35, 14090). Such enzymatic nucleic acid molecules herein are said to “maintain” the enzymatic activity of an all RNA enzymatic nucleic acid molecules.

Therapeutic nucleic acid molecules (e.g., enzymatic nucleic acid molecules, siRNA/siNA, and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and enzymatic nucleic acid stability. In this invention, the product of these properties is increased or not significantly (less that 10 fold) decreased in vivo compared to an all RNA enzymatic nucleic acid molecule.

In one embodiment, the nucleic acid molecules comprises a 5′ and/or a 3′-cap structure.

By “cap structure” is meant chemical modifications, which have been incorporated at the terminus of the oligonucleotide (see for example Wincott et al, WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both terminus. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Beigelman et al, International PCT publication No. WO 97/26270, incorporated by reference herein).

In yet another embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moeity; 5′-5′-inverted abasic moeity; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moeities (for more details see Beaucage and Tyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

By “nucleotide” as used herein is as recognized in the art to include natural bases (standard), and modified bases well known in the art. Such bases are generally located at the 1′ position of a sugar moiety. A nucleotide generally comprises a base, sugar and a phosphate group. The nucleotide may also be abasic, i.e., lacking a base. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; all hereby incorporated by reference herein). Several examples of modified nucleic acid bases are known in the art and has recently been summarized by Limbach et al., 1994, Nucleic Acids Res. 22, 2183. Some of the non-limiting examples of base modifications that can be introduced into enzymatic nucleic acids without significantly effecting their catalytic activity include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine) and others (Burgin et al., 1996, Biochemistry, 35, 14090).

By “modified bases” in this aspect is meant nucleotide bases other than adenine, guanine, cytosine and uracil at 1′ position or their equivalents; such bases may be used within the catalytic core of the enzyme and/or in the substrate-binding regions.

By “abasic” is meant sugar moieties lacking a base or having other chemical groups in place of a base at the 1′ position.

By “unmodified nucleoside” is meant one of the bases adenine, cytosine, guanine, uracil joined to the 1′ carbon of β-D-ribo-furanose.

By “modified nucleoside” is meant any nucleotide base that contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications enhance shelf-life, half-life in vitro, stability, and ease of introduction of such oligonucleotides to the target site, e.g., to enhance penetration of cellular membranes, and confer the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different ribozyme motifs) and/or other chemical or biological molecules). The treatment of subjects with nucleic acid molecules can also include combinations of different types of nucleic acid molecules. Therapies can be devised which include a mixture of enzymatic nucleic acid molecules (including different ribozyme motifs), antisense and/or 2-5A chimera molecules to one or more targets to alleviate symptoms of a disease.

Administration of Nucleic Acid Molecules

Sullivan, et al., supra, describes the general methods for delivery of nucleic acid molecules. Nucleic acid molecules of the invention can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules can be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra and Draper et al., supra which have been incorporated by reference herein.

Methods for the delivery of nucleic acid molecules is described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules can be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra and Draper et al, PCT WO93/23569 which have been incorporated by reference herein.

The nucleic acid molecules of the instant invention can be used as pharmaceutical agents. Pharmaceutical agents prevent, inhibit the occurrence, or treat (alleviate a symptom to some extent, preferably all of the symptoms) of a disease state in a subject.

The nucleic acid molecules of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the cancer cells.

The invention also features the use of a composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). Long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. Id. at 1449. These include sodium benzoate, sorbic acid and esters of p-hydroxybenzoic acid. In addition, antioxidants and suspending agents can be used. Id.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Another means of accumulating high concentrations of a nucleic acid molecule of the invention (e.g., ribozyme or antisense) within cells is to incorporate the nucleic acid-encoding sequences into a DNA or RNA expression vector. Transcription of the nucleic acid sequences are driven from a promoter for eukaryotic RNA polymerase I (pol I), RNA polymerase II (pol II), or RNA polymerase II (po 111I). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990 Proc. Natl. Acad. Sci. USA, 87, 6743-7; Gao and Huang 1993 Nucleic Acids Res., 21, 2867-72; Lieber et al., 1993 Methods Enzymol., 217, 47-66; Zhou et al., 1990 Mol. Cell. Biol., 10, 4529-37; Thompson et al., 1995 supra). Several investigators have demonstrated that enzymatic nucleic acid or antisese expressed from such promoters can function in mammalian cells (e.g. Izant and Weintraub, 1985 Science 229, 345; McGarry and Lindquist, 1986 Proc. Natl. Acad. Sci. USA 83, 399; Kashani-Sabet et al., 1992 Antisense Res. Dev., 2, 3-15; Ojwang et al., 1992 Proc. Natl. Acad. Sci. USA, 89, 10802-6; Chen et al., 1992 Nucleic Acids Res., 20, 4581-9; Yu et al., 1993 Proc. Natl. Acad. Sci. USA, 90, 6340-4; L'Huillier et al., 1992 EMBO J. 11, 4411-8; Lisziewicz et al., 1993 Proc. Natl. Acad. Sci. U.S. A., 90, 8000-4; Thompson et al., 1995 Nucleic Acids Res. 23, 2259). The above nucleic acid transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors).

In one embodiment of the invention, a transcription unit expressing an enzymatic nucleic acid that cleaves RNAs that encode flt-1, KDR and/or flk-1 are inserted into a plasmid DNA vector or an adenovirus or adeno-associated virus DNA viral vector or a retroviral RNA vector. Viral vectors have been used to transfer genes and lead to either transient or long term gene expression (Zabner et al., 1993 Cell 75, 207; Carter, 1992 Curr. Opi. Biotech. 3, 533). The adenovirus, AAV or retroviral vector is delivered as recombinant viral particles. The DNA may be delivered alone or complexed with vehicles (as described for RNA above). The recombinant adenovirus or AAV or retroviral particles are locally administered to the site of treatment, eg., through incubation or inhalation in vivo or by direct application to cells or tissues ex vivo. Retroviral vectors have also been used to express enzymatic nucleic acid in mammalian cells (Ojwang et al., 1992 supra; Thompson et al., 1995 supra).

Flt-1, KDR and/or flk-1 are attractive nucleic acid-based therapeutic targets by several criteria. The interaction between VEGF and VEGF-R is well-established. Efficacy can be tested in well-defined and predictive animal models. Finally, the disease conditions are serious and current therapies are inadequate. Whereas protein-based therapies would inhibit VEGF activity nucleic acid-based therapy provides a direct and elegant approach to directly modulate flt-1, KDR and/or flk-1 expression.

Because flt-1 and KDR mRNAs are highly homologous in certain regions, some enzymatic nucleic acid target sites are also homologous (see Table X). In this case, a single enzymatic nucleic acid cantarget both flt-1 and KDR mRNAs. At partially homologous sites, a single enzymatic nucleic acid can sometimes be designed to accommodate a site on both mRNAs by including G/U base pairing. For example, if there is a G present in an enzymatic nucleic acid target site in KDR mRNA at the same position there is an A in the flt-1 ribozyme target site, the enzymatic nucleic acid can be synthesized with a U at the complementary position and it will bind both to sites. The advantage of one enzymatic nucleic acid that targets both VEGF-R mRNAs is clear, especially in cases where both VEGF receptors may contribute to the progression of angiogenesis in the disease state.

“Angiogenesis” refers to formation of new blood vessels, which is an essential process in reproduction, development and wound repair. “Tumor angiogenesis” refers to the induction of the growth of blood vessels from surrounding tissue into a solid tumor. Tumor growth and tumor metastasis are dependent on angiogenesis (for a review see Folkman, 1985 supra; Folkman 1990 J. Natl. Cancer Inst., 82, 4; Folkman and Shing, 1992 J. Biol. Chem. 267, 10931).

Angiogenesis plays an important role in other diseases such as arthritis wherein new blood vessels have been shown to invade the joints and degrade cartilage (Folkman and Shing, supra).

“Retinopathy” refers to inflammation of the retina and/or degenerative condition of the retina which may lead to occlusion of the retina and eventual blindness. In “diabetic retinopathy” angiogenesis causes the capillaries in the retina to invade the vitreous resulting in bleeding and blindness which is also seen in neonatal retinopathy (for a review see Folkman, 1985 supra; Folkman 1990 supra; Folkman and Shing, 1992 supra).

The following examples further illustrate the present invention but should not be construed to limit the present invention in any way.

### EXAMPLE 1

### flt-1, KDR and/or flk-1 Ribozymes

By engineering ribozyme motifs, Applicant has designed several ribozymes directed against flt-1, KDR and/or flk-1 encoded mRNA sequences. These ribozymes were synthesized with modifications that improve their nuclease resistance (Beigelman et al., 1995 J. Biol. Chem. 270, 25702) and enhance their activity in cells. The ability of ribozymes to cleave target sequences in vitro was evaluated essentially as described in Thompson et al., PCT Publication No. WO 93/23057; Draper et al., PCT Publication No. WO 95/04818.

### EXAMPLE 2

### Effect of Ribozymes on the Binding of VEGF to flt-1, KDR and/or flk-1 Receptors

Several common human cell lines are available that express endogenous flt-1, KDR and/or flk-1. flt-1, KDR and/or flk-1 which can be detected easily with monoclonal antibodies. Use of appropriate fluorescent reagents and fluorescence-activated cell-sorting (FACS) permit direct quantitation of surface flt-1, KDR and/or flk-1 on a cell-by-cell basis. Active ribozymes are expected to directly reduce flt-1, KDR and/or flk-1 expression and thereby reduce VEGF binding to the cells. In this example, human umbelical cord microvascular endothelial cells were used.

Cell Preparation:

Plates were coated with 1.5% gelatin and allowed to stand for one hour. Cells (e.g., microvascular endothelial cells derived from human umbilical cord vein) were plated at 20,000 cells/well (24 well plate) in 200 μl growth media and incubated overnight (˜1 doubling) to yield ˜40,000 cells (75-80% confluent).

Ribozyme Treatment:

Media was removed from cells and the cells were washed two times with 300 μl 1×PBS: Ca²⁺: Mg²⁺ mixture. A complex of 200-500 nM ribozyme and LipofectAMINE® (3:1 lipid:phosphate ratio) in 200 μl OptiMEM® (5% FBS) was added to the cells. The cells were incubated for 6 hr (equivalent to 2-3 VEGF-R turnovers).

¹²⁵I VEGF Binding Assay:

The assay was carried out on ice to inhibit internalization of VEGF during the experiment. The media containing the ribozyme was removed from the cells and the cells were washed twice with 300 μl 1×PBS: Ca²⁺: Mg²⁺ mixture containing 1% BSA. Appropriate ¹²⁵I VEGF solution (100,000 cpm/well, +/−10× cold 1×PBS, 1% BSA) was applied to the cells. The cells were incubated on ice for 1 hour. ¹²⁵I VEGF-containing solution was removed and the cells were washed three times with 300 μl 1×PBS: Ca²⁺: Mg²⁺ mixture containing 1% BSA. To each well 300 μl of 100 mM Tris-HCl, pH 8.0, 0.5% Triton X-100 was added and the mixture was incubated for 2 minutes. The ¹²⁵I VEGF-binding was quantitated using standard scintillation counting techniques. Percent inhibition was calculated as follows:

Percent Inhibition=

$$\frac{cpm {}^{125} I VEGF bound by the ribozyme \text{-} treated samples}{cpm {}^{125} I VEGF bound by the Control sample} \times 100$$

### EXAMPLE 3

### Effect of Hammerhead Ribozymes Targeted Against flt-1 Receptor on the Binding of VEGF

Hammerhead ribozymes targeted to twenty sites within flt-1 RNA were synthesized as described above. The sequences of the ribozymes used are shown in Table II; the length of the stem II region is 3 bp. The hammerhead ribozymes were chemically modified such that the ribozyme consists of ribose residues at five positions; U4 and U7 positions contain 2′-NH₂ modifications, the remaining nucleotide positions contain 2′-O-methyl substitutions; four nucleotides at the 5′ terminus contains phosphorothioate substitutions. Additionally, 3′ end of the ribozyme contains a 3′-3′ linked inverted abasic ribose.

Referring to FIG. 7, the effect of hammerhead ribozymes targeted against flt-1 receptor on the binding of VEGF to flt-1 on the surface of human microvascular endothelial cells is shown. The majority of the ribozymes tested were able to inhibit the expression of flt-1 and thereby were able to inhibit the binding of VEGF.

In order to determine the specificity of ribozymes targeted against flt-1 RNA, the effect of five anti-flt-1 ribozymes on the binding of VEGF, UPA (urokinase plasminogen activator) and FGF (fibroblast growth factor) to their corresponding receptors were assayed. As shown in FIG. 9, there was significant inhibition of VEGF binding to its receptors on cells treated with anti-flt-1 ribozymes. There was no specific inhibition of the binding of UPA and FGF to their corresponding receptors. These data strongly suggest that anti-flt-1 ribozymes specifically cleave flt-1 RNA and not RNAs encoding the receptors for UPA and FGF, resulting in the inhibition of flt-1 receptor expression on the surface of the cells. Thus the ribozymes are responsible for the inhibition of VEGF binding but not the binding of UPA and FGF.

### EXAMPLE 4

### Effect of Hammerhead Ribozymes Targeted Against KDR Receptor on the Binding of VEGF

Hammerhead ribozymes targeted to twenty-one sites within KDR RNA were synthesized as described above. The sequences of the ribozymes used are shown in Table IV; the length of stem II region is 3 bp. The hammerhead ribozymes were chemically modified such that the ribozyme consists of ribose residues at five positions; U4 and U7 positions contain 2′-NH₂ modifications, the remaining nucleotide positions contain 2′-O-methyl substitutions; four nucleotides at the 5′ terminus contains phosphorothioate substitutions. Additionally, the 3′ end of the ribozyme contains a 3′-3′ linked inverted abasic deoxyribose.

Referring to FIG. 8, the effect of hammerhead ribozymes targeted against KDR receptor on the binding of VEGF to KDR on the surface of human microvascular endothelial cells is shown. A majority of the ribozymes tested were able to inhibit the expression of KDR and thereby were able to inhibit the binding of VEGF. As a control, the cells were treated with a ribozyme that is not targeted towards KDR RNA (irrel. RZ); there was no specific inhibition of VEGF binding. The results from this control experiment strongly suggest that the inhibition of VEGF binding observed with anti-KDR ribozymes is a ribozyme-mediated inhibition.

### EXAMPLE 5

### Effect of Ribozymes Targeted Against VEGF Receptors on Cell Proliferation

Cell Preparation:

24-well plates were coated with 1.5% gelatin (porcine skin 300 bloom). After 1 hour, excess gelatin is washed off of the plate. Microvascular endothelial cells were plated at 5,000 cells/well (24 well plate) in 200 μl growth media. The cells were allowed to grow for ˜18 hours (˜1 doubling) to yield ˜10,000 cells (25-30% confluent).

Ribozyme Treatment:

Media was removed from the cells, and the cells were washed two times with 300 μl 1×PBS: Ca²⁺: Mg²⁺ mixture.

For anti-flt-1HH ribozyme experiment (FIG. 12) a complex of 500 nM ribozyme; 15 μM LFA (3:1 lipid:phosphate ratio) in 200 μl OptiMEM (5% FCS) media was added to the cells. Incubation of cells was carried out for 6 hours (equivalent to 2-3 VEGF receptor turnovers).

For anti-KDR HH ribozyme experiment (FIG. 13) a complex of 200 nM ribozyme; 5.25 μM LFA (3:1 lipid:phosphate ratio) in 200 μl OptiMEM (5% FCS) media was added to the cells. Incubation of cells was carried out for 3 hours.

Proliferation:

After three or six hours, the media was removed from the cells and the cells were washed with 300 μl 1×PBS: Ca²⁺: Mg²⁺ mixture. Maintenance media (contains dialyzed 10% FBS)+/− VEGF or basic FGF at 10 ng/ml was added to the cells. The cells were incubated for 48 or 72 hours. The cells were trypsinized and counted (Coulter counter). Trypan blue was added on one well of each treatment as a control.

As shown in FIG. 12B, VEGF and basic FGF stimulate human microvascular endothelial cell proliferation. However, treatment of cells with 1358 HH or 4229 HH ribozymes, targeted against flt-1 mRNA, results in a significant decrease in the ability of VEGF to stimulate endothelial cell proliferation. These ribozymes do not inhibit the FGF-mediated stimulation of endothelial cell proliferation.

Human microvascular endothelial cells were also treated with hammerhead ribozymes targeted against sites 527, 730, 3702 or 3950 within the KDR mRNA. As shown in FIG. 13, all four ribozymes caused significant inhibition of VEGF-mediated induction of cell proliferation. No significant inhibition of cell proliferation was observed when the cells were treated with a hammerhead ribozyme targeted to an irrelevant RNA. Additionally, none of the ribozymes inhibited FGF-mediated stimulation of cell proliferation.

These results strongly suggest that hammerhead ribozymes targeted against either flt-1 or KDR mRNA specifically inhibit VEGF-mediated induction of endothelial cell proliferation.

### EXAMPLE 6

### Effect of Antisense Oligonucleotides Targeted against VEGF Receptors on Cell Proliferation (Colorimetric Assay)

The following are some of the reagents used in the proliferation assay:

Cells: Human aortic endothelial cells (HAEC) from Clonetics®. Cells at early passage are preferably used.

Uptake Medium: EBM (from Clonetics®)); 1% L-Glutamine; 20 mM Hepes; No serum; No antibiotics.

Growth Medium: EGM (from Clonetics®); FBS to 20%; 1% L-Glutamine; 20 mM Hepes.

Cell Plating: 96-well tissue culture plates were coated with 0.2% gelatin (50 μl/well). The gelatin was incubated in the wells at room temperature for 15-30 minutes. The gelatin was removed by aspiration and the wells were washed with PBS:Ca²⁺: Mg²⁺ mixture. PBS mixture was left in the wells until cells were ready to be added. HAEC cells were detached by trypsin treatment and resuspended at 1.25×10⁴/ml in growth medium. PBS was removed from plates and 200 μl of cells (i.e. 2.5×10³ cells/well) were added to each well. The cells were allowed to grow for 48 hours before the proliferation assay.

Assay: Growth medium was removed from the wells. The cells were washed twice with PBS:Ca²⁺: Mg²⁺ mixture without antibiotics. A formulation of lipid/antisense oligonucleotide (antisense oligonucleotide is used here as a non-limiting example) complex was added to each well (100 μl/well) in uptake medium. The cells were incubated for 2-3 hours at 37° C. in a CO₂ incubator. After uptake, 100 μl/well of growth medium was added (gives final FBS concentration of 10%). After approximately 72 hours, 40 μl MTS® stock solution (made as described by manufacturer) was added to each well and incubated at 37° C. for 1-3 hours, depending on the color development. (For this assay, 2 hours was sufficient). The intensity of color formation was determined on a plate reader at 490 nM.

Phosphorothioate-substituted antisense oligodeoxynucleotides were custom synthesized by The Midland Certified Reagent Company®, Midland, Tex. Following non-limiting antisense oligodeoxynucleotides targeted against KDR RNA were used in the proliferation assay:

KDR 21 AS:
5′-GCA GCA CCT TGC TCT CCA TCC-3′
SCRAMBLED CONTROL:
5′-CTG CCA ACT TCC CAT GCC TGC-3′

As shown in FIG. 10, proliferation of HAEC cells is specifically inhibited by increasing concentrations of the phosphorothioate anti-KDR-antisense oligodeoxynucleotide. The scrambled antisense oligonucleotide is not expected to bind the KDR RNA and therefore is not expected to inhibit KDR expression. As expected, there is no detectable inhibition of proliferation of HAEC cells treated with a phosphorothioate antisense oligonucleotide with scrambled sequence.

### EXAMPLE 7

### In Vitro Cleavage of flt-1 RNA by Hammerhead Ribozymes

Referring to FIG. 11A, hammerhead ribozymes (HH) targeted against sites 1358 and 4229 within the flt-1 RNA were synthesized as described above.

RNA Cleavage Assay In Vitro:

Substrate RNA was 5′ end-labeled using [γ-³²P] ATP and T4 polynucleotide kinase (US Biochemicals). Cleavage reactions were carried out under ribozyme “excess” conditions. Trace amount (≦1 nM) of 5′ end-labeled substrate and 40 nM unlabeled ribozyme were denatured and renatured separately by heating to 90° C. for 2 minutes and snap-cooling on ice for 10-15 minutes. The ribozyme and substrate were incubated, separately, at 37° C. for 10 minutes in a buffer containing 50 mM Tris-HCl and 10 mM MgCl₂. The reaction was initiated by mixing the ribozyme and substrate solutions and incubating at 37° C. Aliquots of 5 μl were taken at regular intervals of time and the reaction was quenched by mixing with equal volume of 2× formamide stop mix. The samples were resolved on 20% denaturing polyacrylamide gels. The results were quantified and percentage of target RNA cleaved is plotted as a function of time.

Referring to FIGS. 11B and 11C, hammerhead ribozymes targeted against sites 1358 and 4229 within the flt-1 RNA are capable of cleaving target RNA efficiently in vitro.

### EXAMPLE 8

### In Vitro Cleavage of KDR RNA by Hammerhead Ribozymes

In this non-limiting example, hammerhead ribozymes targeted against sites 726, 527, 3702 and 3950 within KDR RNA were synthesized as described above. RNA cleavage reactions were carried out in vitro essentially as described under Example 7.

Referring to FIGS. 14 and 15, all four ribozymes were able to cleave their cognate target RNA efficiently in a sequence-specific manner.

### EXAMPLE 9

### In Vitro Cleavage of RNA by Hammerhead Ribozymes Targeted Against Cleavage Sites that are Homologous between KDR and flt-1 mRNA

Given that flt-1 and KDR mRNAs are highly homologous in certain regions, some ribozyme target sites are also homologous (see Table X). In this case, a single ribozyme will target both flt-1 and KDR mRNAs. Hammerhead ribozyme (FLT/KDR-I) targeted against one of the homologous sites between flt-1 and KDR (flt-1 site 3388 and KDR site 3151) was synthesized as described above. Ribozymes with either a 3 bp stem II or a 4 bp stem II were synthesized. RNA cleavage reactions were carried out in vitro essentially as described under Example 7.

Referring to FIG. 16, FLT/KDR-I ribozyme with either a 3 or a 4 bp stem II was able to cleave its target RNA efficiently in vitro.

### EXAMPLE 10

### Effect of Multiple Ribozymes Targeted Against both flt-1 and KDR RNA on Cell Proliferation

Since both flt-1 and KDR receptors of VEGF are involved in angiogenesis, the inhibition of the expression of both of these genes can be an effective approach to inhibit angiogenesis.

Human microvascular endothalial cells were treated with hammerhead ribozymes targeted against sites flt-1 4229 alone, KDR 527 alone, KDR 726 alone, KDR 3950 alone, flt-1 4229+KDR 527, flt-1 4229+KDR 726 or flt-1 4229+KDR 3950. As shown in FIG. 17, all the combinations of active ribozymes (A) caused significant inhibition of VEGF-mediated induction of cell proliferation. No significant inhibition of cell proliferation was observed when the cells were treated with a catalytically inactive (I) hammerhead ribozymes. Additionally, cells treated with ribozymes targeted against both fit-1 and KDR RNAs-flt-1 4229+KDR 527; flt-1 4229+KDR 726; flt-1 4229+KDR 3950, were able to cause a greater inhibition of VEGF-mediated induction of cell proliferation when compared with individual ribozymes targeted against either flt-1 or KDR RNA (see flt-1 4229 alone; KDR 527 alone; KDR 726 alone; KDR 3950 alone). This strongly suggests that treatment of cells with multiple ribozymes can be a more effective means of inhibition of gene expression.

Animal Models

There are several animal models in which the anti-angiogenesis effect of nucleic acids of the present invention, such as enzymatic nucleic acids, directed against VEGF-R mRNAs can be tested. Typically a corneal model has been used to study angiogenesis in rat and rabbit since recruitment of vessels can easily be followed in this normally avascular tissue (Pandey et al., 1995 Science 268: 567-569). In these models, a small Teflon or Hydron disk pretreated with an angiogenesis factor (e.g. bFGF or VEGF) is inserted into a pocket surgically created in the cornea. Angiogenesis is monitored 3 to 5 days later. Enzymatic nucleic acids directed against VEGF-R mRNAs are delivered in the disk as well, or dropwise to the eye over the time course of the experiment. In another eye model, hypoxia has been shown to cause both increased expression of VEGF and neovascularization in the retina (Pierce et al., 1995 Proc. Natl. Acad. Sci. USA. 92: 905-909; Shweiki et al., 1992 J. Clin. Invest. 91: 2235-2243).

In human glioblastomas, it has been shown that VEGF is at least partially responsible for tumor angiogenesis (Plate et al., 1992 Nature 359, 845). Animal models have been developed in which glioblastoma cells are implanted subcutaneously into nude mice and the progress of tumor growth and angiogenesism is studied (Kim et al., 1993 supra; Millauer et al., 1994 supra).

Another animal model that addresses neovascularization involves Matrigel, an extract of basement membrane that becomes a solid gel when injected subcutaneously (Passaniti et al., 1992 Lab. Invest. 67: 519-528). When the Matrigel is supplemented with angiogenesis factors such as VEGF, vessels grow into the Matrigel over a period of 3 to 5 days and angiogenesis can be assessed. Again, nucleic acids directed against VEGF-R mRNAs are delivered in the Matrigel.

Several animal models exist for screening of anti-angiogenic agents. These include corneal vessel formation following corneal injury (Burger et al., 1985 Cornea 4: 35-41; Lepri, et al., 1994 J. Ocular Pharmacol. 10: 273-280; Ormerod et al., 1990 Am. J. Pathol. 137: 1243-1252) or intracorneal growth factor implant (Grant et al., 1993 Diabetologia 36: 282-291; Pandey et al. 1995 supra; Zieche et al., 1992 Lab. Invest. 67: 711-715), vessel growth into Matrigel matrix containing growth factors (Passaniti et al., 1992 supra), female reproductive organ neovascularization following hormonal manipulation (Shweiki et al., 1993 Clin. Invest. 91: 2235-2243), several models involving inhibition of tumor growth in highly vascularized solid tumors (O'Reilly et al., 1994 Cell 79: 315-328; Senger et al., 1993 Cancer and Metas. Rev. 12: 303-324; Takahasi et al., 1994 Cancer Res. 54: 4233-4237; Kim et al., 1993 supra), and transient hypoxia-induced neovascularization in the mouse retina (Pierce et al., 1995 Proc. Natl. Acad. Sci. USA. 92: 905-909).

The cornea model, described in Pandey et al. supra, is the most common and well characterized anti-angiogenic agent efficacy screening model. This model involves an avascular tissue into which vessels are recruited by a stimulating agent (growth factor, thermal or alkalai burn, endotoxin). The corneal model utilizes the intrastromal corneal implantation of a Teflon pellet soaked in a VEGF-Hydron solution to recruit blood vessels toward the pellet which can be quantitated using standard microscopic and image analysis techniques. To evaluate their anti-angiogenic efficacy, nucleic acids are applied topically to the eye or bound within Hydron on the Teflon pellet itself. This avascular cornea as well as the Matrigel (see below) provide for low background assays. While the corneal model has been performed extensively in the rabbit, studies in the rat have also been conducted.

The mouse model (Passaniti et al., supra) is a non-tissue model which utilizes Matrigel, an extract of basement membrane (Kleinman et al., 1986) or Millipore® filter disk, which can be impregnated with growth factors and anti-angiogenic agents in a liquid form prior to injection. Upon subcutaneous administration at body temperature, the Matrigel or Millipore® filter disk forms a solid implant. VEGF embedded in the Matrigel or Millipore® filter disk is used to recruit vessels within the matrix of the Matrigel or Millipore® filter disk which can be processed histologically for endothelial cell specific vWF (factor VII antigen) immunohistochemistry, Trichrome-Masson stain, or hemoglobin content. Like the cornea, the Matrigel or Millipore® filter disk are avascular; however, it is not tissue. In the Matrigel or Millipore® filter disk model, nucleic acids are administered within the matrix of the Matrigel or Millipore® filter disk to test their anti-angiogenic efficacy. Thus, delivery issues in this model, as with delivery of nucleic acids by Hydron-coated Teflon pellets in the rat cornea model, may be less problematic due to the homogeneous presence of the nucleic acid within the respective matrix.

These models offer a distinct advantage over several other angiogenic models listed previously. The ability to use VEGF as a pro-angiogenic stimulus in both models is highly desirable since the instant nucleic acid molecules target only VEGFr mRNA. In other words, the involvement of other non-specific types of stimuli in the cornea and Matrigel models is not advantageous from the standpoint of understanding the pharmacologic mechanism by which the anti-VEGFr mRNA nucleic acid molecules produce their effects. In addition, the models allow for testing the specificity of the anti-VEGFr mRNA nucleic acids by using either a- or bFGF as a pro-angiogenic factor. Vessel recruitment using FGF should not be affected in either model by anti-VEGFr mRNA nucleic acid molecules. Other models of angiogenesis including vessel formation in the female reproductive system using hormonal manipulation (Shweiki et al., 1993 supra); a variety of vascular solid tumor models which involve indirect correltations with angiogenesis (O'Reilly et al., 1994 supra; Senger et al., 1993 supra; Takahasi et al., 1994 supra; Kim et al., 1993 supra); and retinal neovascularization following transient hypoxia (Pierce et al., 1995 supra) were not selected for efficacy screening due to their non-specific nature, although there is a correlation between VEGF and angiogenesis in these models.

Other model systems to study tumor angiogenesis is reviewed by Folkman, 1985 Adv. Cancer. Res. 43, 175.

Use of Murine Models

For a typical systemic study involving 10 mice (20 g each) per dose group, 5 doses (1, 3, 10, 30 and 100 mg/kg daily over 14 days continuous administration), approximately 400 mg of enzymatic nucleic acid, formulated in saline is used. A similar study in young adult rats (200 g) requires over 4 g. Parallel pharmacokinetic studies involve the use of similar quantities of enzymatic nucleic acid further justifying the use of murine models.

Enzymatic Nucleic Acids and Lewis Lung Carcinoma and B-16 Melanoma Murine Models

Identifying a common animal model for systemic efficacy testing of enzymatic nucleic acid is an efficient way of screening enzymatic nucleic acid for systemic efficacy.

The Lewis lung carcinoma and B-16 murine melanoma models are well accepted models of primary and metastatic cancer and are used for initial screening of anti-cancer agents. These murine models are not dependent upon the use of immunodeficient mice, are relatively inexpensive, and minimize housing concerns. Both the Lewis lung and B-16 melanoma models involve subcutaneous implantation of approximately 10⁶ tumor cells from metastatically aggressive tumor cell lines (Lewis lung lines 3LL or D122, LLc-LN7; B-16-BL6 melanoma) in C57BL/6J mice. Alternatively, the Lewis lung model can be produced by the surgical implantation of tumor spheres (approximately 0.8 mm in diameter). Metastasis also can be modeled by injecting the tumor cells directly intravenously. In the Lewis lung model, microscopic metastases can be observed approximately 14 days following implantation with quantifiable macroscopic metastatic tumors developing within 21-25 days. The B-16 melanoma exhibits a similar time course with tumor neovascularization beginning 4 days following implantation. Since both primary and metastatic tumors exist in these models after 21-25 days in the same animal, multiple measurements can be taken as indices of efficacy. Primary tumor volume and growth latency as well as the number of micro- and macroscopic metastatic lung foci or number of animals exhibiting metastases can be quantitated. The percent increase in lifespan can also be measured. Thus, these models provide suitable primary efficacy assays for screening systemically administered enzymatic nucleic acids and enzymatic nucleic acid formulations.

In the Lewis lung and B-16 melanoma models, systemic pharmacotherapy with a wide variety of agents usually begins 1-7 days following tumor implantation/inoculation with either continuous or multiple administration regimens. Concurrent pharmacokinetic studies can be performed to determine whether sufficient tissue levels of ribozymes can be achieved for pharmacodynamic effect to be expected. Furthermore, primary tumors and secondary lung metastases can be removed and subjected to a variety of in vitro studies (i.e. target RNA reduction).

Flt-1, KDR and/or flk-1 protein levels can be measured clinically or experimentally by FACS analysis. Flt-1, KDR and/or flk-1 encoded mRNA levels are assessed by Northern analysis, RNase-protection, primer extension analysis and/or quantitative RT-PCR. Nucleic acids that block flt-1, KDR and/or flk-1 protein encoding mRNAs and therefore result in decreased levels of flt-1, KDR and/or flk-1 activity by more than 20% in vitro can be identified.

Nucleic acids and/or genes encoding them are delivered by either free delivery, liposome delivery, cationic lipid delivery, adeno-associated virus vector delivery, adenovirus vector delivery, retrovirus vector delivery or plasmid vector delivery in these animal model experiments (see above).

Subjects can be treated by locally administering nucleic acids targeted against VEGF-R by direct injection. Routes of administration include, but are not limited to, intravascular, intramuscular, subcutaneous, intraarticular, aerosol inhalation, oral (tablet, capsule or pill form), topical, systemic, ocular, intraperitoneal and/or intrathecal delivery.

### EXAMPLE 11

### Ribozyme-Mediated Inhibition of Angiogenesis In Vivo

The purpose ot this study was to assess the anti-angiogenic activity of hammerhead ribozymes targeted against flt-1 4229 site in the rat cornea model of VEGF induced angiogenesis (see above). These ribozymes have either active or inactive catalytic core and either bind and cleave or just bind to VEGF-R mRNA of the flt-1 subtype. The active ribozymes, that are able to bind and cleave the target RNA, have been shown to inhibit (¹²⁵I-labeled) VEGF binding in cultured endothelial cells and produce a dose-dependent decrease in VEGF induced endothelial cell proliferation in these cells (see Examples 3-5 above). The catalytically inactive forms of these ribozymes, wherein the ribozymes can only bind to the RNA but cannot catalyze RNA cleavage, fail to show these characteristics. The ribozymes and VEGF were co-delivered using the filter disk method: Nitrocellulose filter disks (Millipore®) of 0.057 diameter were immersed in appropriate solutions and were surgically implanted in rat cornea as described by Pandey et al., supra. This delivery method has been shown to deliver rhodamine-labeled free ribozyme to scleral cells and, in all likelihood cells of the pericorneal vascular plexus. Since the active ribozymes show cell culture efficacy and can be delivered to the target site using the disk method, it is essential that these ribozymes be assessed for in vivo anti-angiogenic activity.

The stimulus for angiogenesis in this study was the treatment of the filter disk with 30 μM VEGF which is implanted within the cornea's stroma. This dose yields reproducible neovascularization stemming from the pericorneal vascular plexus growing toward the disk in a dose-response study 5 days following implant. Filter disks treated only with the vehicle for VEGF show no angiogenic response. The ribozymes were co-adminstered with VEGF on a disk in two different ribozyme concentrations. One concern with the simultaneous administration is that the ribozymes will not be able to inhibit angiogenesis since VEGF receptors can be stimulated. However, Applicant has observed that in low VEGF doses, the neovascular response reverts to normal suggesting that the VEGF stimulus is essential for maintaining the angiogenic response. Blocking the production of VEGF receptors using simultaneous administration of anti-VEGF-R mRNA ribozymes could attenuate the normal neovascularization induced by the filter disk treated with VEGF.

Materials and Methods:

1. Stock Hammerhead Ribozyme Solutions:

a. flt-1 4229 (786 μM)—Active

b. flt-1 4229 (736 μM)—Inactive

2. Experimantal Solutions/Groups:

| | | |
|---|---|---|
| Group 1 | Solution 1 | Control VEGF solution: 30 μM in 82 mM Tris base |
| Group 2 | Solution 2 | flt-1 4229 (1 μg/μL) in 30 μM |
| | | VEGF/82 mM Tris base |
| Group 3 | Solution 3 | flt-1 4229 (10 μg/μL) in 30 μM |
| | | VEGF/82 mM Tris base |
| Group 4 | Solution 4 | No VEGF, flt-1 4229 (10 μg/μL) |
| | | in 82 mM Tris base |
| Group 5 | Solution 5 | No VEGF, No ribozyme in 82 mM Tris base |

10 eyes per group, 5 animals (Since they have similar molecular weights, the molar concentrations should be essentially similar).

Each solution (VEGF and RIBOZYMES) were prepared as a 2× solution for 1:1 mixing for final concentrations above, with the exception of solution 1 in which VEGF was 2× and diluted with ribozyme diluent (sterile water).

3. VEGF Solutions

The 2×VEGF solution (60 μM) was prepared from a stock of 0.82 μg/μL in 50 mM Tris base. 200 μL of VEGF stock was concentrated by speed vac to a final volume of 60.8 μL, for a final concentration of 2.7 μg/μL or 60 μM. Six 10 μL aliquots were prepared for daily mixing. 2× solutions for VEGF and Ribozyme was stored at 4° C. until the day of the surgery. Solutions were mixed for each day of surgery. Original 2× solutions were prepared on the day before the first day of the surgery.

4. Surgical Solutions:

Anesthesia:

stock ketamine hydrochloride 100 mg/mL

stock xylazine hydrochloride 20 mg/mL

stock acepromazine 10 mg/mL

Final anesthesia solution: 50 mg/mL ketamine, 10 mg/mL xylazine, and 0.5 mg/mL acepromazine

5% povidone iodine for opthalmic surgical wash

2% lidocaine (sterile) for opthalmic administration (2 drops per eye)

sterile 0.9% NaCl for opthalmic irrigation

5. Surgical Methods:

Standard surgical procedure was performed as described in Pandey et al., supra. Filter disks were incubated in 1 μL of each solution for approximately 30 minutes prior to implantation.

6. Experimental Protocol:

The animal corneas were treated with the treatment groups as described above. Animals were allowed to recover for 5 days after treatment with daily observation (scoring 0-3). On the fifth day animals were euthanized and digital images of each eye was obtained for quantitaion using Image Pro Plus. Quantitated neovascular surface areas were analyzed by ANOVA followed by two post-hoc tests including Dunnets and Tukey-Kramer tests for significance at the 95% confidence level. Dunnets provide information on the significance between the differences within the means of treatments vs. controls while Tukey-Kramer provide information on the significance of differences within the means of each group.

Results are graphically represented in FIG. 18. As shown in FIG. 18, flt-1 4229 active hammerhead ribozyme at both concentrations was effective at inhibiting angiogenesis, while the inactive ribozyme did not show any significant reduction in angiogenesis. A statistically signifiant reduction in neovascular surface area was observed only with active ribozymes. This result clearly shows that the ribozymes are capable of significantly inhibiting angiogenesis in vivo. Specifically, the mechanism of inhibition appears to be by the binding and cleavage of target RNA by ribozymes.

### EXAMPLE 12

### Bioactivity of Anti-Angiogenesis Ribozymes Targeting flt-1 and kdr RNA

Materials and Methods

Ribozymes: Hammerhead ribozymes and controls designed to have attenuated activity (attenuated controls) were synthesized and purified as previously described above. The attenuated ribozyme controls maintain the binding arm sequence of the parent ribozyme and thus are still capable of binding to the mRNA target. However, they have two nucleotide changes in the core sequence that substantially reduce their ability to carry out the cleavage reaction. Ribozymes were designed to target Flt-1 or KDR mRNA sites conserved in human, mouse, and rat. In general, ribozymes with binding arms of seven nucleotides were designed and tested. If, however, only six nucleotides surrounding the cleavage site were conserved in all three species, six nucleotide binding arms were used. A subset of ribozyme and attenuated control sequences and modifications are listed in Table XII. Data are presented herein for 2′-NH₂ uridine modified ribozymes in cell proliferation studies and for 2′-C-allyl uridine modified ribozymes in RNAse protection, in vitro cleavage and corneal studies.

In vitro ribozyme cleavage assays: In vitro RNA cleavage rates on a 15 nucleotide synthetic RNA substrate were measured as previously described above.

Cell culture: Human dermal microvascular endothelial cells (HMVEC-d, Clonetics Corp.) were maintained at 37° C. in flasks or plates coated with 1.5% porcine skin gelatin (300 bloom, Sigma) in Growth medium (Clonetics Corp.) supplemented with 10-20% fetal bovine serum (FBS, Hyclone). Cells were grown to confluency and used up to the seventh passage. Stimulation medium consisted of 50% Sigma 99 media and 50% RPMI 1640 with L-glutamine and additional supplementation with 10 μg/mL Insulin-Transferrin-Selenium (Gibco BRL) and 10% FBS. Cell growth was stimulated by incubation in Stimulation medium supplemented with 20 ng/mL of either VEGF₁₆₅ or bFGF. VEGF₁₆₅ (165 amino acids) was selected for cell culture and animal studies because it is the predominant form of the four native forms of VEGF generated by alternative mRNA splicing. Cell culture assays were carried out in triplicate.

Ribozyme and Ribozyme/LIPOFECTAMINE™ Formulations:

Cell culture: Ribozymes or attenuated controls (50-200 nM) were formulated for cell culture studies and used immediately. Formulations were carried out with LIPOFECTAMINE™(Gibco BRL) at a 3:1 lipid to phosphate charge ratio in serum-free medium (OPTI-MEM™, Gibco BRL) by mixing for 20 minutes at room temperature. For example, a 3:1 lipid to phosphate charge ratio was established by complexing 200 nM ribozyme with 10.8 μg/L LIPOFECTAMINE™ (13.5 μM DOSPA).

In vivo: For corneal studies, lyophilized ribozyme or attenuated controls were resuspended in sterile water at a final stock concentration of 170 μg/μL (highest dose). Lower doses (1.7-50 μg/μL) were prepared by serial dilution in sterile water.

Proliferation assay: HMVEC-d were seeded (5×10³ cells/well) in 48-well plates (Costar) and incubated 24-30 hours in Growth medium at 37° C. After removal of the Growth medium, cells were treated with 50-200 nM LIPOFECTAMINE™ complexes of ribozyme or attenuated controls for 2 hours in OPTI-MEM™. The ribozyme/control-containing medium was removed and the cells were washed extensively in 1×PBS. The medium was then replaced with Stimulation medium or Stimulation medium supplemented with 20 ng/mL VEGF₁₆₅ or bFGF. After 48 hours, the cell number was determined using a Coulter™ cell counter. Data are presented as cell number per well following 48 h of VEGF stimulation.

RNAse protection assay: HMVEC-d were seeded (2×10⁵ cells/well) in 6-well plates (Costar) and allowed to grow 32-36 hours in Growth medium at 37° C. Cells were treated with LIPOFECTAMINE™ complexes containing 200 nM ribozyme or attenuated control for 2 hours as described under “Proliferation Assay” and then incubated in Growth medium containing 20 ng/mL VEGF₁₆₅ for 24 hours. Cells were harvested and an RNAse protection assay was carried out using the Ambion Direct Protect kit and protocol with the exception that 50 mM EDTA was added to the lysis buffer to eliminate the possibility of ribozyme cleavage during sample preparation. Antisense RNA probes targeting portions of Flt-1 and KDR were prepared by transcription in the presence of [³²P]-UTP. Samples were analyzed on polyacrylamide gels and the level of protected RNA fragments was quantified using a Molecular Dynamics PhosphorImager. The levels of Flt-1 and KDR were normalized to the level of cyclophilin (human cyclophilin probe template, Ambion) in each sample. The coefficient of variation for cyclophilin levels was 11% [265940 cpm±1 29386 (SD)] for all conditions tested here (i.e. in the presence of either active ribozymes or attenuated controls). Thus, cyclophilin is useful as an internal standard in these studies.

Rat Corneal Pocket assay Of VEGF-Induced Angiogenesis:

Animal guidelines and anesthesia. Animal housing and experimentation adhered to standards outlined in the 1996 Guide for the Care and Use of Laboratory Animals (National Research Council). Male Sprague Dawley rats (250-300 g) were anesthetized with ketamine (50 mg/kg), xylazine (10 mg/kg), and acepromazine (0.5 mg/kg) administered intramuscularly (im). The level of anesthesia was monitored every 2-3 min by applying hind limb paw pressure and examining for limb withdrawal. Atropine (0.4 mg/kg, im) was also administered to prevent potential corneal reflex-induced bradycardia.

Preparation of VEGF soaked disk. For corneal implantation, 0.57 mm diameter nitrocellulose disks, prepared from 0.45 μm pore diameter nitrocellulose filter membranes (Millipore Corporation), were soaked for 30 min in 1 μL of 30 μM VEGF₁₆₅ in 82 mM Tris HCl (pH 6.9) in covered petri dishes on ice.

Corneal surgery. The rat corneal model used in this study was a modified from Koch et al.

Supra and Pandey et al., supra. Briefly, corneas were irrigated with 0.5% povidone iodine solution followed by normal saline and two drops of 2% lidocaine. Under a dissecting microscope (Leica MZ-6), a stromal pocket was created and a presoaked filter disk (see above) was inserted into the pocket such that its edge was 1 mm from the corneal limbus.

Intraconjunctival injection of test solutions. Immediately after disk insertion, the tip of a 40-50 μm OD injector (constructed in our laboratory) was inserted within the conjunctival tissue 1 mm away from the edge of the corneal limbus that was directly adjacent to the VEGF-soaked filter disk. Six hundred nanoliters of test solution (ribozyme, attenuated control or sterile water vehicle) were dispensed at a rate of 1.2 μL/min using a syringe pump (Kd Scientific). The injector was then removed, serially rinsed in 70% ethanol and sterile water and immersed in sterile water between each injection. Once the test solution was injected, closure of the eyelid was maintained using microaneurism clips until the animal began to recover gross motor activity. Following treatment, animals were warmed on a heating pad at 37° C.

Animal treatment groups/experimental protocol. Ribozymes targeting Flt-1 site 4229 and KDR mRNA site 726 were tested in the corneal model along with their attenuated controls. Five treatment groups were assigned to examine the effects of five doses of each test substance over a dose range of 1-100 μg on VEGF-stimulated angiogenesis. Negative (30 μM VEGF soaked filter disk and intraconjunctival injection of 600 nL sterile water) and no stimulus (Tris-soaked filter disk and intraconjunctival injection of sterile water) control groups were also included. Each group consisted of five animals (10 eyes) receiving the same treatment.

Quantitation of angiogenic response. Five days after disk implantation, animals were euthanized following im administration of 0.4 mg/kg atropine and corneas were digitally imaged. The neovascular surface area (NSA, expressed in pixels) was measured postmortem from blood-filled corneal vessels using computerized morphometry (Image Pro Plus, Media Cybernetics, v2.0). The individual mean NSA was determined in triplicate from three regions of identical size in the area of maximal neovascularization between the filter disk and the limbus. The number of pixels corresponding to the blood-filled corneal vessels in these regions was summated to produce an index of NSA. A group mean NSA was then calculated. Data from each treatment group were normalized to VEGF/ribozyme vehicle-treated control NSA and finally expressed as percent inhibition of VEGF-induced angiogenesis.

Statistics. After determining the normality of treatment group means, group mean percent inhibition of VEGF-induced angiogenesis was subjected to a one-way analysis of variance. This was followed by two post-hoc tests for significance including Dunnett's (comparison to VEGF control) and Tukey-Kramer (all other group mean comparisons) at alpha=0.05. Statistical analyses were performed using JMP v.3.1.6 (SAS Institute).

RESULTS.

Ribozyme-mediated reduction of VEGF-induced cell proliferation: Ribozyme cleavage of Flt-1 or KDR mRNA should result in a decrease in the density of cell surface VEGF receptors. This decrease should limit VEGF binding and consequently interfere with the mitogenic signaling induced by VEGF. To determine if cell proliferation was impacted by anti-Flt-1 and/or anti-KDR ribozyme treatment, proliferation assays using cultured human microvascular cells were carried out. Ribozymes included in the proliferation assays were initially chosen by their ability to decrease the level of VEGF binding to treated cells (see FIG. 8). In these initial studies, ribozymes targeting 20 sites in the coding region of each mRNA were screened. The most effective ribozymes against two sites in each target (Table XII), Flt-1 sites 1358 and 4229 and KDR sites 726 and 3950, were included in the proliferation assays reported here (FIG. 19). In addition, attenuated analogs of each ribozyme were used as controls (Table XII). These attenuated controls are still capable of binding to the mRNA target since the binding arm sequence is maintained. However, these controls have two nucleotide changes in the core sequence that substantially reduce their ability to carry out the cleavage reaction.

The antiproliferative effect of active ribozymes targeting two lead sites on each VEGF receptor mRNA is shown in FIG. 19. The active ribozymes tested decreased the relative proliferation of HMVEC-d after VEGF stimulation, an effect that increased with ribozyme concentration. This concentration dependency was not observed following treatment with the attenuated controls designed for these sites. In fact, little or no change in cell growth was noted following treatment with the attenuated controls, even though these controls can still bind to the specific target sequences. At 200 nM, there was a distinct “window” between the anti-proliferative effects of each ribozyme and its attenuated control; a trend also observed at lower doses. This window of inhibition of proliferation (56-77% based on total cells/well) reflects the contribution of ribozyme-mediated activity. In comparison, no effect of anti-Flt-1 or anti-KDR ribozymes was noted on bFGF-stimulated cell proliferation (FIGS. 19C, 19F). Moreover, an irrelevant, but active, ribozyme whose binding sequence is not found in either Flt-1 or KDR mRNA had no effect in this assay (FIG. 19B). These data are consistent with the basic ribozyme mechanism in which binding and cleavage are necessary components. Although the relative surface distribution of Flt-1 and KDR receptors in this cell type is not known, the antiproliferative effects of these ribozymes indicate that, at least in cell culture, both receptors are functionally coupled to proliferation.

Specific reduction of Flt-1 or KDR mRNA by ribozyme treatment: To confirm that anti-Flt-1 and anti-KDR ribozymes reduce their respective mRNA targets, cellular levels of Flt-1 or KDR were quantified using an RNAse protection assay with specific Flt-1 or KDR probes. For each target, one ribozyme/attenuated control pair was chosen for continued study. Data from a representative experiment are shown in FIG. 20. Exposure of HMVEC-d to active ribozyme targeting Flt-1 site 4229 decreased Flt-1 mRNA, but not KDR mRNA. Likewise, treatment with the active ribozyme targeting KDR site 726 decreased KDR, but not Flt-1 mRNA. Both ribozymes decreased the level of their respective target RNA by greater than 50%. The degree of reduction associated with the corresponding attenuated controls was not greater than 13%.

In Vitro Activity of Anti-Flt and Anti-KDR Ribozymes.

To confirm further the necessity of an active ribozyme core, in vitro cleavage activities were determined for the Flt-1 site 4229 ribozyme and the KDR site 726 ribozyme as well as their paired attenuated controls. The first order rate constants calculated from the time-course of short substrate cleavage for the anti-Flt-1 ribozyme and its attenuated control were 0.081±0.0007 min⁻¹ and 0.001±6×10⁻⁵ min⁻¹, respectively. For the anti-KDR ribozyme and its paired control, the first order rate constants were 0.434±0.024 min⁻¹ and 0.002±1×10⁻⁴ min⁻¹, respectively. Although the attenuated controls retain a very slight level of cleavage activity under these optimized conditions, the decrease in in vitro cleavage activity between each active ribozyme and its paired attenuated control is about two orders of magnitude. Thus, an active core is essential for cleavage activity in vitro and is also necessary for ribozyme activity in cell culture.

Ribozyme-mediated reduction of VEGF-induced angiogenesis in vivo. To assess whether ribozymes targeting VEGF receptor mRNA could impact the complex process of angiogenesis, prototypic anti-Flt-1 and KDR ribozymes that were identified in cell culture studies were screened in a rat corneal pocket assay of VEGF-induced angiogenesis. In this assay, corneas implanted with VEGF-containing filter disks exhibited a robust neovascular response in the corneal region between the disk and the corneal limbus (from which the new vessels emerge). Disks containing a vehicle solution elicited no angiogenic response. In separate studies, intraconjunctival injections of sterile water vehicle did not affect the magnitude of the VEGF-induced angiogenic response. In addition, ribozyme injections alone did not induce angiogenesis.

The dose-related effects of anti-Flt-1 or KDR ribozymes on the VEGF-induced angiogenic response were then examined. FIGS. 21 and 22 illustrate the quantified antiangiogenic effect of the anti-Flt-(site 4229) and KDR (site 726) ribozymes and their attenuated controls over a dose range from 1 to 100 μg, respectively. For both ribozymes, the maximal antiangiogenic response (48 and 36% for anti-Flt-1 and KDR ribozymes, respectively) was observed at a dose of 10 μg.

The anti-Flt-1 ribozyme produced a significantly greater antiangiogenic response than its attenuated control at 3 and 10 μg (p<0.05; FIG. 21). Its attenuated control exhibited a small but significant antiangiogenic response at doses above 10 μg compared to vehicle treated VEGF controls (p<0.05; FIG. 21). At its maximum, this response was not significantly greater than that observed with the lowest dose of active anti-Flt-1 ribozyme. The anti-KDR ribozyme significantly inhibited angiogenesis from 3 to 30 μg (p<0.05; FIG. 22). The anti-KDR attenuated control had no significant effect at any dose tested.

### EXAMPLE 13

### In Vivo Inhibition of Tumor Growth and Metastases by VEGF-R Ribozymes.

A. Lewis Lung Carcinoma Mouse Model: Ribozymes were chemically synthesized as described above. The sequence of ANGIOZYME™ bound to its target RNA is shown in FIG. 26.

The tumors in this study were derived from a cell line (LLC-HM) which gives rise to reproducible numbers of spontaneous lung metastases when propagated in vivo. The LLC-HM line was obtained from Dr. Michael O'Reilly, Harvard University. Tumor neovascularization in Lewis lung carcinoma has been shown to be VEGF-dependent. Tumors from mice bearing LLC-HM (selected for the highly metastatic phenotype by serial propagation) were harvested 20 days post-inoculation. A tumor brei suspension was prepared from these tumors according to standard protocols. On day 0 of the study, 0.5×10⁶ viable LLC-HM tumor cells were injected subcutaneously (sc) into the dorsum or flank of previously untreated mice (100 μL injectate). Tumors were allowed to grow for a period of 3 days prior to initiating continuous intravenous administration of saline or 30 mg/kg/d ANGIOZYME™ via Alzet mini-pumps. One set of animals was dosed from days 3 to 17, inclusive. Tumor length and width measurements and volumes were calculated according to the formula: Volume=0.5(length)(width)². At post-inoculation day 25, animals were euthanized and lungs harvested. The number of lung macrometastatic nodules was counted. It should be noted that metastatic foci were quantified 8 days after the cessation of dosing. Ribozyme solutions were prepared to deliver to another set of animals 100, 10, 3, or 1 mg/kg/day of ANGIOZYME™ via Alzet mini-pumps. A total of 10 animals per dose or saline control group were surgically implanted on the left flank with osmotic mini-pumps pre-filled with the respective test solution three days following tumor inoculation. Pumps were attached to indwelling jugular vein catheters.

FIG. 27 shows the antitumor effects of ANGIOZYME™. There is a statistically significant inhibition (p<0.05) of primary LLC-HM tumor growth in tumors grown in the flank regions compared to saline control. ANGIOZYME™ significantly reduced (p<0.05) the number of lung metastatic foci in animals inoculated either in the flank regions. FIG. 28 illustrates the dose-dependent anti-metastatic effect of ANGIOZYME™ compared to saline control.

B. Mouse Colorectal Cancer Model. KM12L4a-16 is a human colorectal cancer cell line. On day 0 of the study, 0.5×10⁶ KM12L4a-16 cells were implanted into the spleen of nude mice. Three days after tumor inoculation, Alzet minipumps were implanted and continuous subcutaneous delivery of either saline or 12, 36 or 100 mg/kg/day of ANGIOZYME™ was initiated. On day 5, the spleens containing the primary tumors were removed. On day 18, the Alzet minipumps were replaced with fresh pumps so that delivery of saline or ANGIOZYME™ was continuous over a 28 day period from day 3 to day 32. Animals were euthanized on day 41 and the liver tumor burden was evaluated.

Following treatment with 100 mg/kg/day of ANGIOZYME™, there was a significant reduction in the incidence and median number of liver metastasis (FIGS. 29 and 30). In saline-treated animals, the median number of metastases was ≧101. However, at the high dose of ANGIOZYME™ (100 mg/kg/day), the median number of metastases was zero.

### EXAMPLE 14

### Effect of ANGIOZYME™ Alone or in Combination with Chemotherapeutic Agents in the Mouse Lewis Lung Carcinoma Model.

Methods

Tumor inoculations. Male C57/BL6 mice, age 6 to 8 weeks, were inoculated subcutaneously in the flank with 5×10⁵ LLC-HM cells from brei preparations made from tumors grown in mice.

Ribozymes and controls. The ribozyme and controls tested in this study are given in Table XIII. RPI.4610, also known as ANGIOZYME™, is an anti-Flt-1 ribozyme that targets site 4229 in the human Flt-1 receptor mRNA (EMBL accession no. X51602). The controls tested include RPI.13141, an attenuated version of RPI.4610 in which four nucleotides in the catalytic core are changed so that the cleavage activity is dramatically decreased. RPI.13141, however, maintains the base composition and binding arms of RPI.4610 and so is still capable of binding to the target site. The second control (RPI.13030) also has changes to the catalytic core (three) to inhibit cleavage activity, but in addition the sequence of the binding arms has been scrambled so that it can no longer bind to the target sequence. One nucleotide in the arm of RPI.13030 is also changed to maintain the same base composition as RPI.4610.

Ribozyme administrations. Ribozymes and controls were resuspended in normal saline. Administration was initiated seven days following tumor inoculation. Animals either received a daily subcutaneous injection (30 mg/kg test substance) from day 7 to day 20 or were instrumented with an Alzet osmotic minipump (12 μL/day flow rate) containing a solution of ribozyme or control. Subcutaneous infusion pumps delivered the test substances (30 mg/kg/day) from day 7 to 20 (14-day pumps, 420 mg/kg total test substance) or days 7-34 (28-day pumps, 840 mg/kg total test substance). Where indicated, chemotherapeutic agents were given in combination with ribozyme treatment. Cyclophosphamide was given by ip administration on days 7, 9 and 11 (125 mg/kg). Gemcitabine was given by intraperitoneal administration on days 8, 11 and 14 (125 mg/kg). Untreated, uninstrumented animals were used as comparison. Five animals were included in each group.

Results

The antiangiogenic ribozyme, ANGIOZYME™, was tested in a model of Lewis lung carcinoma alone and in combination with two chemotherapeutic agents. Previously (see above), 30 mg/kg/day ANGIOZYME™ alone was determined to inhibit both primary tumor growth and lung metastases in a highly metastatic variant of Lewis lung (continuous 14-day intraveneous delivery via Alzet minipump).

In this study, 30 mg/kg/day ANGIOZYME™ delivered either as a daily subcutaneous bolus injection or as a continuous infusion from an Alzet minipump resulted in a delay in tumor growth (FIG. 23). On average, tumor growth to 500 mm³ was delayed by approximately 7 days in animals being treated with ANGIOZYME™ compared to an untreated group. Growth of tumors in animals being treated with either of two attenuated controls was delayed by only approximately 2 days.

ANGIOZYME™ delivered by subcutaneous bolus was also tested in combination with either Gemcytabine or cyclophosphamide (FIG. 24). Tumor growth delay increased by about 3 days in the presence of combination therapy with ANGIOZYME™ and Gemcytabine over the effects of either treatment alone. The combination of ANGIOZYME™ and cyclophosphamide did not increase tumor growth delay over that of cyclophosphamide alone, however, suboptimal doses of cyclophosphamide were not included in this study. Neither of the attenuated controls increased the effect of the chemotherapeutic agents.

The effect of ANGIOZYME™ on metastases to the lung was also determined in the presence and absence of additional chemotherapeutic treatment. Macrometastases to the lungs were counted in two animals in each treatment group on day 20. Data for the daily subcutaneous administration of 30 mg/kg ANGIOZYME™ alone or with Gemcytabine or cyclophosphamide is given in FIG. 25. In the presence of ANGIOZYME™, with or without a chemotherapeutic agent, the lung metastases were reduced to zero. Treatment with either Gemcytabine or cyclophosphamide alone (mean number of metastases 4.5 and 4, respectively) were not as effective as ANGIOZYME™ alone or when used in combination with ANGIOZYME™. Neither of the attenuated controls increased the effect of the chemotherapeutic agents.

The effect on metastases to the lung was also determined following continuous treatment with ANGIOZYME™. At day 20, an average of approximately 8 macrometastases were noted in the treatment groups which had been instrumented with Alzet minipumps (either 14- or 28-day pumps). This is a decrease in metastases of approximately 50% from the untreated group. Since ANGIOZYME™ delivered by a daily subcutaneous bolus resulted in zero metastases (FIG. 4) in the two animals counted, it is possible that the additional burden of being instrumented with the minipump contributes to a slightly decreased response to ANGIOZYME™.

### EXAMPLE 15

### Phase I/II Study of Repetitive Dose ANGIOZYME™ Targeting the FLT-1 Receptor of VEGF

A ribozyme therapeutic agent ANGIOZYME™, was assessed by daily subcutaneous administration in a phase I/II trial for 31 subjects with refractory solid tumors. Demographic information relating to subjects enrolled in the study are shown in Table XX. The primary study endpoint was to determine the safety and maximum tolerated dose of ANGIOZYME™. Secondary endpoints assessed ANGIOZYME™ pharmacokinetics and clinical response. Subjects were treated in four cohorts of three subjects at doses of 10, 30, 100, and 300 mg/mZ/day. Following the dose escalation phase, an additional 15 evaluable subjects were entered in an expanded cohort at 100 mg/m2/day. Subjects were dosed for a minimum of 29 consecutive days with 24-hour pharmacokinetic analyses on Day 1 and 29. Clinical response was assessed monthly.

Results The data from 20 subjects indicated that ANGIOZYME™ was well tolerated, with no systemic adverse events. FIG. 31 shows the plasma concentration profile of ANGIOZYME™ after a single SC (sub-cutaneous) dose of 10, 30, 100, or 300 mg/m². The pharmacokinetic parameters of ANGIOZYME™ after SC bolus administration are outlined in Table XXI. An MTD (maximum tolerated dose) could not be established. One subject in the 300 mg/m²/d group experienced a grade 3 injection site reaction. Subjects in the other groups experienced intermittent grade 1 and grade 2 injection site reactions with erythema and induration. No systemic or laboratory toxicities were observed. Pharmacokinetic analyses demonstrated dose-dependent plasma concentrations with good bioavailability (70-90%), t½=209-384 min, and no accumulation after repeated doses. To date, 17/28 (61%) of evaluable subjects have had stable disease for periods of one to six months and two subjects (nasopharyngeal squamous cell carcinoma and melanoma) had minor clinical responses. The subject with nasopharyngeal carcinoma demonstrated central tumor necrosis as indicated by MRI. The longest period of treatment thus far has been 8 months for two subjects at 100 mg/m²/d (breast, peritoneal mesothelioma).

### EXAMPLE 16

### In Vivo Inhibition of Neovascularization in an Ocular Animal Model by VEGF-R Ribozymes

Summary of the Mouse Model: A mouse model of proliferative retinopathy (Aiello et al., 1995, Proc. Natl. Acad. Sci. USA 92: 10457-10461; Robinson et al., 1996, Proc. Natl. Acad. Sci. USA 93: 4851-4856; Pierce et al., 1996, Archives of Ophthalmology 114: 1219-1228) in which neovascularization of the mouse retina is induced by exposure of 7-day old mice to 75% oxygen followed by a return to normal room air. The initial period in high oxygen causes an obliteration of developing blood vessels in the retina. Exposure to room air five days later is perceived as hypoxia by the now underperfused retina. The result is an immediate upregulation of VEGF mRNA and VEGF protein (between 6-12 hours) followed by an extensive retinal neovascularization that peaks in approximately 5 days. Although this model is more representative of retinopathy of prematurity than diabetic retinopathy, it is an accepted small animal model in which to study neovascular pathophysiology of the retina. In fact, intravitreal injection of certain antisense DNA constructs targeting VEGF mRNA have been found to be antiangiogenic in this model, as were soluble VEGF receptor chimeric proteins designed to bind VEGF in the vitreous humor (Aiello et al., 1995, Proc. Natl. Acad. Sci. USA 92: 10457-10461; Robinson et al., 1996, Proc. Natl. Acad. Sci. USA 93: 4851-4856; Pierce et al., 1996, Archives of Ophthalmology 114: 1219-1228).

Summary of experiment: The effect of an anti-KDR/Flk-1 ribozyme on the peak level of neovascularization was tested in the mouse model described above. As shown in FIG. 34A, P7 mice were removed from the hyperoxic chamber and the mice received two intraocular injections (P12 and P13) in the right eye of 10 μg RPI.4731, the anti-KDR/Flk-1 ribozyme. The left eye of each mouse was treated as a control and received intraocular injections of saline. Five days after being exposed to room air, neovascular nuclei in the retina of both eyes were counted. Data are presented in FIG. 34B. There was a significant decrease in retinal neovascularization (approximately 40%) compared to the control, saline-injected eyes.

RPI.4731 sequence and chemical composition:

5′-uₛaₛcₛaₛau ucU GAu Gag gcg aaa gcc Gaa Aag aca aB-3′ (SEQ ID NO: 13488)

where:

uppercase G, A=ribonucleotides

lowercase=2′-OMe

U=2′-C-allyl uridine

B=inverted abasic nucleotide

S=phosphorothioate linkage

### EXAMPLE 17

### Therapeutic Effects of Anti-flt-1 and Anti-kdr Ribozymes Against Primary and Metastatic 4T1 Murine Mammary Carcinoma

Cleavage of flt-1 and/or kdr mRNA should result in down regulation of VEGF receptor subtypes 1 and 2, respectively. As described in the Examples above, treatment of animals with a ribozyme that specifically binds to and cleaves flt-1 mRNA (ANGIOZYME) results in reduced primary tumor growth and/or decreased metastases in murine models of lung (LLC-HM murine lung carcinoma) and colorectal carcinoma (KM12L4a). The purpose of this study was to determine the effects of treatment with ANGIOZYME and an anti-kdr ribozyme in a murine model of mammary carcinoma that spontaneously metastasizes.

Methods: The 4T1 murine mammary carcinoma (syngeneic to BALB/c mice) forms progressive subcutaneous (SC) tumors that spontaneously metastasize to regional lymph nodes, lung, liver and brain in a manner similar to human breast cancer. Mice were inoculated with 4T1 tumor cells either subcutaneously or in the hindlimb footpad and primary tumor growth, number of pulmonary metastases and overall survival were assessed. ANGIOZYME and the anti-kdr ribozyme was administered at varying doses and schedules.

Results: Table 24 shows the number of spontaneous pulmonary metastases resulting from 4T1 tumor inoculation in mice treated with ANGIOZYME compares to controls. Mice treated with ANGIOZYME alone showed no significant delay in 3-day established SC tumor growth as compared to controls. However, the development of spontaneous pulmonary metastases was significantly reduced in mice treated with ANGIOZYME at either 300 or 600 mg/m²/d (see Table 24). Mice treated with ANGIOZYME on days 3-32, with surgical removal of the primary tumor site on day 14, had a significant survival advantage (60% and 80% long-term cure, respectively) as compared to control mice, mice treated on days 3-14 only, or mice treated following removal of the primary tumor. Although combination treatment with ANGIOZYME and an anti-kdr ribozyme had no additional effect on pulmonary metastases at the doses tested, there was an additive reduction in primary SC tumor growth (see FIG. 35).

Conclusion: In 4T1 metastatic mammary carcinoma models, ANGIOZYME treatment significantly reduced pulmonary metastases and conferred a significant survival benefit compared to control, vehicle treated animals. The combined effects of an anti-kdr ribozyme and ANGIOZYME suggest that targeted down regulation of VEGFR1 and VEGFR2 may provide significant therapeutic advantages in vivo.

### EXAMPLE 18

### Antiangiogenic Activity of ANGIOZYME Combined with Interferon-alpha2b.

IFN-alpha2b and IFN-alpha have been shown to inhibit tumor angiogenesis in human xenografts when administered as single agents. As described in the Examples above, treatment of animals with a ribozyme that specifically binds to and cleaves flt-1 mRNA (ANGIOZYME) results in reduced primary tumor growth and/or decreased metastases in murine models of lung (LLC-HM murine lung carcinoma) and colorectal carcinoma (KM12L4a). Therefore, the combination of interferon and nucleic acid molecules (enzymatic nucleic acid molecules, antisense, siNA) targeting VEGF receptors may have an additive or synergistic effect on inhibition of angiogenesis, thus providing an additional treatment option for diseases and conditions described herein. The purpose of this study was to determine the effects of treatment with ANGIOZYME and IFN-alpha2b in the murine dermis model of angiogenesis.

Methods: The murine dermis model was used to assess the effects of the combined treatment of IFN-alpha2b and ANGIOZYME, a ribozyme directed against the vascular endothelial growth factor receptor-1 (VEGFR-1) mRNA. On day zero, two million tumor cells (human Hey ovarian, ACHN renal, SK-MEL-1 melanoma, or LNCaP prostate) were inoculated in the dermis of the flanks of athymic nude mice. Recombinant IFN-alpha2b (Intron, specific activity 1-2×10⁸ units/mg protein) and ANGIOZYME were used in these studies. Various dosages and schedules of IFN-alpha2b (0.1 mL subcutaneously) and ANGIOZYME (0.1 mL subcutaneously) were used. Mice were sacrificed on day 10 (avertin 0.5 mL intraperitoneally) and the tumor site was assessed for neovascularization by enumeration of radially oriented vessels under a dissecting microscope, measured by an observer blinded as to treatment group.

Results: In all four cell lines, treatment with ANGIOZYME or IFN-alpha2b alone, at doses which did not effect cell proliferation, resulted in a dose-dependent decrease in the mean number of peritumoral vessels. Escalating the ANGIOZYME dose (0.5, 1.0, 2.0 mg/day) resulted in a statistically significant decrease in the number of peritumoral vessels. In Hey ovarian tumors vessel number was reduced by 19%, 45%, 71% following ANGIOZYME dosing of 0.5, 1.0, 2.0 mg/mouse/day, respectively. In all cell lines, daily doses of 10⁴ U IFN-α2b yielded a statistically significant decrease (p<0.05) in vessel counts when compared to control untreated animals. Similarly, 10⁵ U IFN-alpha2b was superior to 10⁴ U in all cell lines (p<0.05). In three of four cell lines, increasing the IFN-alpha2b dose to 10⁶ U did not provide added benefit. Combination treatment with suboptimal doses of ANGIOZYME (0.5 mg/day) and IFN-alpha2b (10⁴ U/day) resulted in synergistic anti-angiogenic activity in three out of four cell lines. In Hey tumors, single agent ANGIOZYME and IFN-alpha2b caused a 25% and 20% reduction, respectively, whereas the combination resulted in a 76% reduction in vessel number. Similarly, in SK-Mel-1 tumors, single agent ANGIOZYME and IFN-alpha2b caused a 17% and 19% reduction, respectively, whereas the combination resulted in a 81% reduction in vessel number. Median effect analysis yielded a combination index of 0.032 (Hey cells) and <0.00001 (SK-Mel-1 cells), indicating strong anti-angiogenic synergy between ANGIOZYME and IFN-alpha2b.

Indications

The nucleic acid molecules discussed herein are useful in the prevention or treatment of the following exemplary conditions related to the level of VEG-F:

1) Tumor angiogenesis: Angiogenesis has been shown to be necessary for tumors to grow into pathological size (Folkman, 1971, PNAS 76, 5217-5221; Wellstein & Czubayko, 1996, Breast Cancer Res and Treatment 38, 109-119). In addition, it allows tumor cells to travel through the circulatory system during metastasis. Increased levels of gene expression of a number of angiogenic factors such as vascular endothelial growth factor (VEGF) have been reported in vascularized and edema-associated brain tumors (Berkman et al., 1993 J. Clini. Invest. 91, 153). A more direct demonstration of the role of VEGF in tumor angiogenesis was demonstrated by Jim Kim et al., 1993 Nature 362,841 wherein, monoclonal antibodies against VEGF were successfully used to inhibit the growth of rhabdomyosarcoma, glioblastoma multiforme cells in nude mice. Similarly, expression of a dominant negative mutated form of the flt-1 VEGF receptor inhibits vascularization induced by human glioblastoma cells in nude mice (Millauer et al., 1994, Nature 367, 576). Specific tumor/cancer types that can be targeted using the nucleic acid molecules of the invention include but are not limited to the tumor/cancer types described under Diagnosis in Table XX.

2) Ocular diseases: Neovascularization has been shown to cause or exacerbate ocular diseases including but not limited to, macular degeneration, neovascular glaucoma, diabetic retinopathy, myopic degeneration, and trachoma (Norrby, 1997, APMIS 105, 417-437). Aiello et al., 1994 New Engl. J. Med. 331, 1480, showed that the ocular fluid, of a majority of subjects suffering from diabetic retinopathy and other retinal disorders, contains a high concentration of VEGF. Miller et al., 1994 Am. J. Pathol. 145, 574, reported elevated levels of VEGF mRNA in subjects suffering from retinal ischemia. These observations support a direct role for VEGF in ocular diseases. Other factors, including those that stimulate VEGF synthesis, may also contribute to these indications.

3) Dermatological Disorders: Many indications have been identified which may be angiogenesis dependent including, but not limited to, psoriasis, verruca vulgaris, angiofibroma of tuberous sclerosis, pot-wine stains, Sturge Weber syndrome, Kippel-Trenaunay-Weber syndrome, and Osler-Weber-Rendu syndrome (Norrby, supra). Intradermal injection of the angiogenic factor b-FGF demonstrated angiogenesis in nude mice (Weckbecker et al., 1992, Angiogenesis: Key principles-Science-Technology-Medicine, ed R. Steiner) Detmar et al., 1994 J. Exp. Med. 180, 1141 reported that VEGF and its receptors were over-expressed in psoriatic skin and psoriatic dermal microvessels, suggesting that VEGF plays a significant role in psoriasis.

4) Rheumatoid arthritis: Immunohistochemistry and in situ hybridization studies on tissues from the joints of subjects suffering from rheumatoid arthritis show an increased level of VEGF and its receptors (Fava et al., 1994 J. Exp. Med. 180, 341). Additionally, Koch et al., 1994 J. Immunol. 152, 4149, found that VEGF-specific antibodies were able to significantly reduce the mitogenic activity of synovial tissues from subjects suffering from rheumatoid arthritis. These observations support a direct role for VEGF in rheumatoid arthritis. Other angiogenic factors including those of the present invention may also be involved in arthritis.

5) Autosomal dominant polycystic kidney disease (ADPKD): ADPKD is the most common life threatening hereditary disease in the USA. It affects about 1:400 to 1:1000 people.

Approximately 50% of people with ADPKD develop renal failure. ADPKD accounts for about 5-10% of end-stage renal failure in the USA requiring dialysis and renal transplantation. The Han:SPRD rat model, mice with a targeted mutation in the Pkd2 gene, and congenital polycystic kidney (cpk) mice closely resemble human ADPKD and present an opportunity to evaluate the therapeutic effect of agents that have the potential to interfere with one or more of the pathogenic elements of ADPKD. One feature of ADPKD is angiogenesis, which may be necessary for growth of cyst cells as well as increased vascular permeability, promoting fluid secretion into cysts. Proliferation of cystic epithelium is also a feature of ADPKD. Cyst cells in culture produce soluble vascular endothelial growth factor (VEGF), which is proven to be specific and critical for blood vessel formation. VEGF is also the best validated target for anti-angiogenesis therapies based on overwhelming genetic, mechanistic and animal efficacy data. However, VEGF can also directly stimulate proliferation of epithelial cells. VEGF triggers a response by interacting with cell-surface receptors. VEGFR1 has been detected in epithelial cells of cystic tubules but not in endothelial cells in the vasculature of cystic kidneys or normal kidneys. VEGFR2 expression is increased in endothelial cells of cyst vessels and in endothelial cells during renal ischemia-reperfusion. It is proposed that inhibition of VEGF receptors with anti-VEGFR1 and anti-VEGFR2 (KDR) agents (eg. nucleic acid molecules of the invention) would attenuate cyst formation, renal failure and mortality in ADPKD. Anti-VEGFR2 agents (eg. nucleic acid molecules of the invention) would inhibit angiogenesis involved in cyst formation. As VEGFR1 is present in cystic epithelium and not in vascular endothelium of cysts, it is proposed that anti-VEGFR1 agents would attenuate cystic epithelial cell proliferation and apoptosis which would in turn lead to less cyst formation. Further, it is proposed that VEGF produced by cystic epithelial cells is one of the stimuli for angiogenesis as well as epithelial cell proliferation and apoptosis. Validation assays for nucleic acid molecules of the invention can be performed in Han:SPRD rats, mice with a targeted mutation in the Pkd2 gene, and cpk mice. The effect of anti-VEGF nucleic acids on cyst formation and renal failure can determine the potential harmful role of angiogenesis in ADPKD.

Combination Therapies

Gemcytabine and cyclophosphamide are non-limiting examples of chemotherapeutic agents that can be combined with or used in conjunction with the nucleic acid molecules (e.g. enzymatic nucleic acid and antisense molecules) of the instant invention to prevent and/or treat VEG-F related conditions. Those skilled in the art will recognize that other anti-angiogenic and/or anti-cancer compounds and therapies can be similarly be readily combined with the nucleic acid molecules of the instant invention (e.g. ribozymes and antisense molecules) and are hence within the scope of the instant invention. Such compounds and therapies are well known in the art (see for example Cancer: Principles and Pranctice of Oncology, Volumes 1 and 2, eds Devita, V. T., Hellman, S., and Rosenberg, S. A., J. B. Lippincott Company, Philadelphia, USA;

incorporated herein by reference) and include, without limitations, folates, antifolates, pyrimidine analogs, fluoropyrimidines, purine analogs, adenosine analogs, topoisomerase I inhibitors, anthrapyrazoles, retinoids, antibiotics, anthacyclins, platinum analogs, alkylating agents, nitrosoureas, plant derived compounds such as vinca alkaloids, epipodophyllotoxins, tyrosine kinase inhibitors, taxols, radiation therapy, surgery, nutritional supplements, gene therapy, radiotherapy, for example 3D-CRT, immunotoxin therapy, for example ricin, and monoclonal antibodies. Specific examples of chemotherapeutic compounds that can be combined with or used in conjuction with the nucleic acid molecules of the invention include, but are not limited to, Paclitaxel; Docetaxel; Methotrexate; Doxorubin; Edatrexate; Vinorelbine; Tomaxifen; Leucovorin; 5-fluoro uridine (5-FU); lonotecan; Cisplatin; Carboplatin; Amsacrine; Cytarabine; Bleomycin; Mitomycin C; Dactinomycin; Mithramycin; Hexamethylmelamine; Dacarbazine; L-asperginase; Nitrogen mustard; Melphalan, Chlorambucil; Busulfan; Ifosfamide; 4-hydroperoxycyclophosphamide, Thiotepa; Irinotecan (CAMPTOSAR®, CPT-11, Camptothecin-11, Campto) Tamoxifen, Hereeptin; IMC C225; ABX-EGF: and combinations thereof.

Diagnostic Uses

The nucleic acid molecules of this invention (e.g., enzymatic nucleic acid) can be used as diagnostic tools to examine genetic drift and mutations within diseased cells or to detect the presence of flt-1, KDR and/or flk-1 RNA in a cell. The close relationship between enzymatic nucleic acid activity and the structure of the target RNA allows the detection of mutations in any region of the molecule which alters the base-pairing and three-dimensional structure of the target RNA. By using multiple enzymatic nucleic acids described in this invention, one can map nucleotide changes which are important to RNA structure and function in vitro, as well as in cells and tissues. Cleavage of target RNAs with enzymatic nucleic acid can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acids targeted to different genes, enzymatic nucleic acids coupled with known small molecule inhibitors, or intermittent treatment with combinations of enzymatic nucleic acids and/or other chemical or biological molecules). Other in vitro uses of enzymatic nucleic acids of the invention are well known in the art, and include detection of the presence of mRNAs associated with flt-1, KDR and/or flk-1related condition. Such RNA is detected by determining the presence of a cleavage product after treatment with a ribozyme using standard methodology.

In a specific example, enzymatic nucleic acids which can cleave only wild-type or mutant forms of the target RNA are used for the assay. The first enzymatic nucleic acid is used to identify wild-type RNA present in the sample and the second enzymatic nucleic acid is used to identify mutant RNA in the sample. As reaction controls, synthetic substrates of both wild-type and mutant RNA are cleaved by both enzymatic nucleic acids to demonstrate the relative enzymatic nucleic acid efficiencies in the reactions and the absence of cleavage of the “non-targeted” RNA species. The cleavage products from the synthetic substrates also serve to generate size markers for the analysis of wild-type and mutant RNAs in the sample population. Thus, each analysis requires two enzymatic nucleic acids, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., flt-1, KDR and/or flk-1) is adequate to establish risk. If probes of comparable specific activity are used for both transcripts, then a qualitative comparison of RNA levels is adequate and decreases the cost of the initial diagnosis. Higher mutant form to wild-type ratios is correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential usefulness of sequence-specific enzymatic nucleic acid molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of Ribozymes

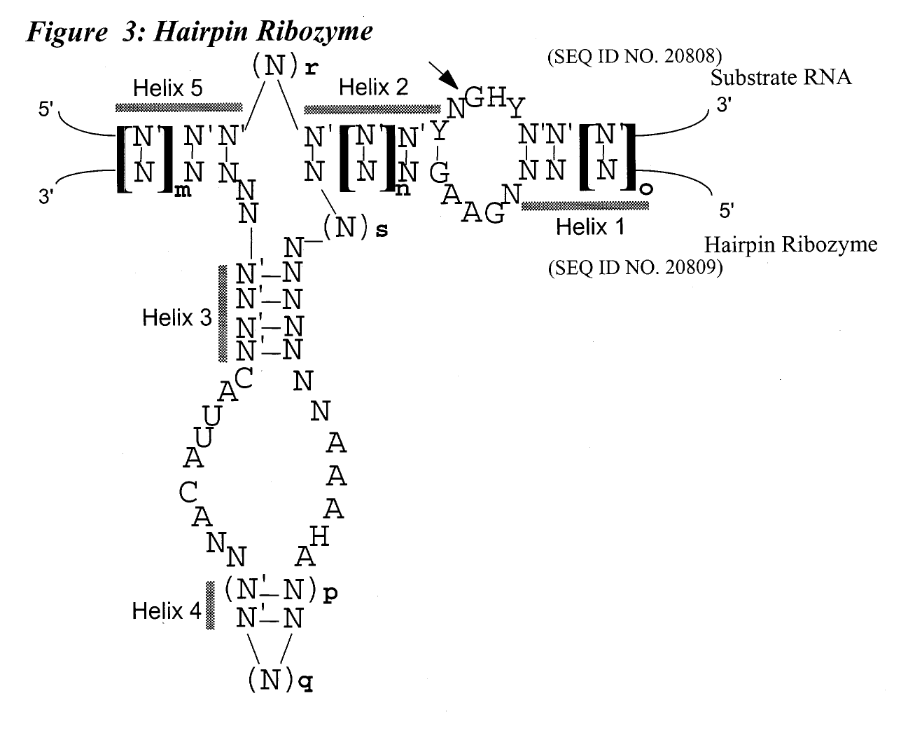

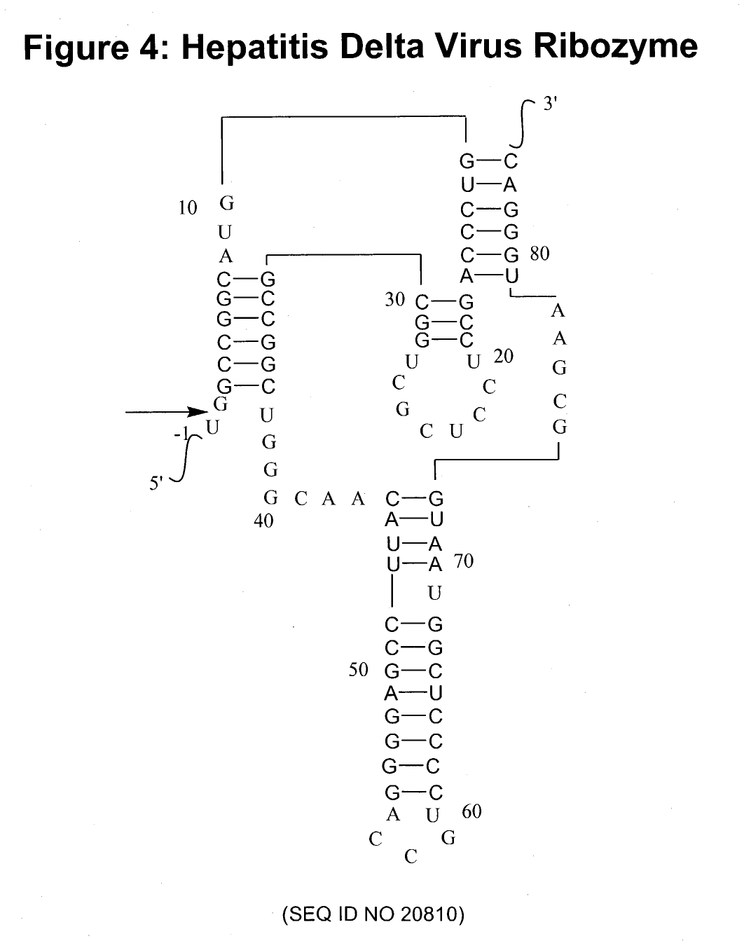

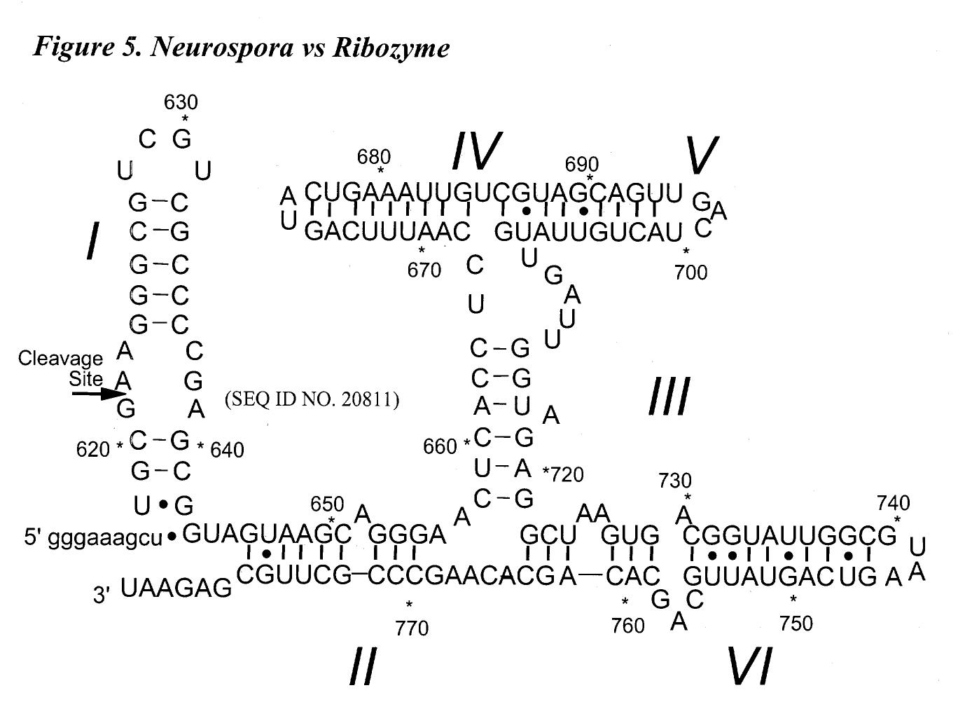

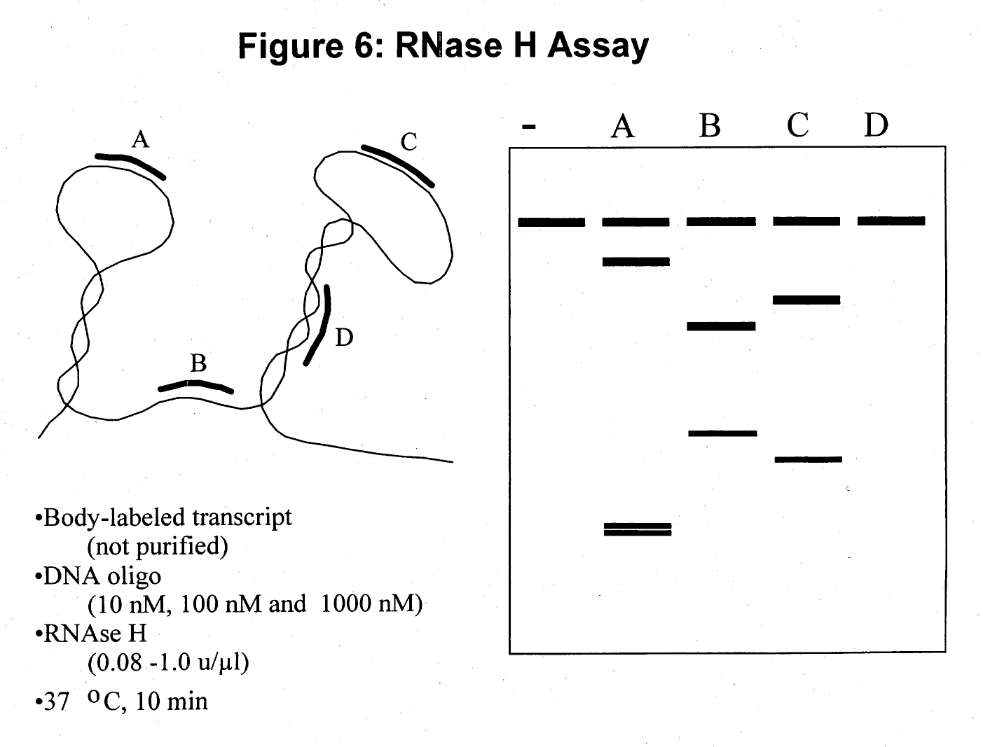

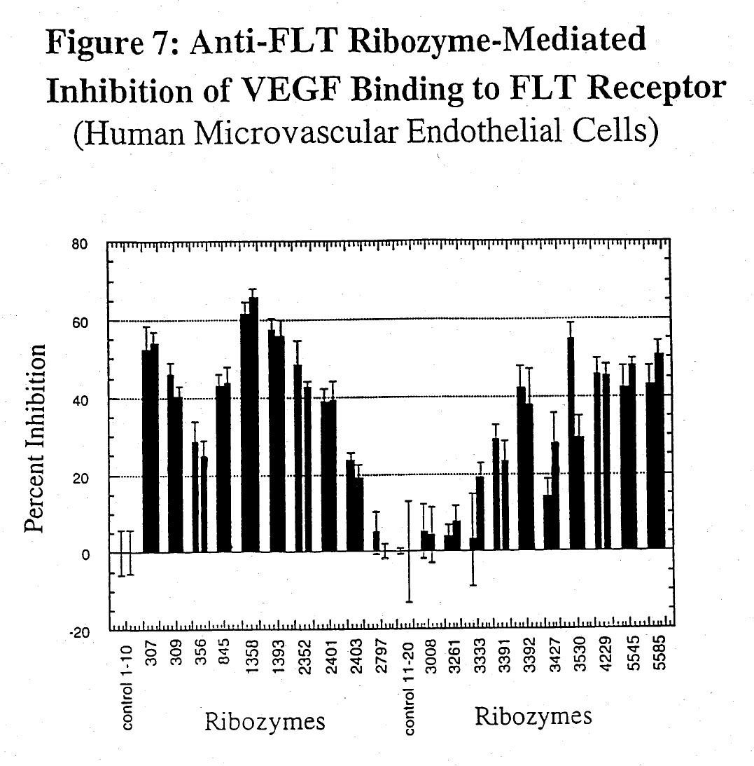

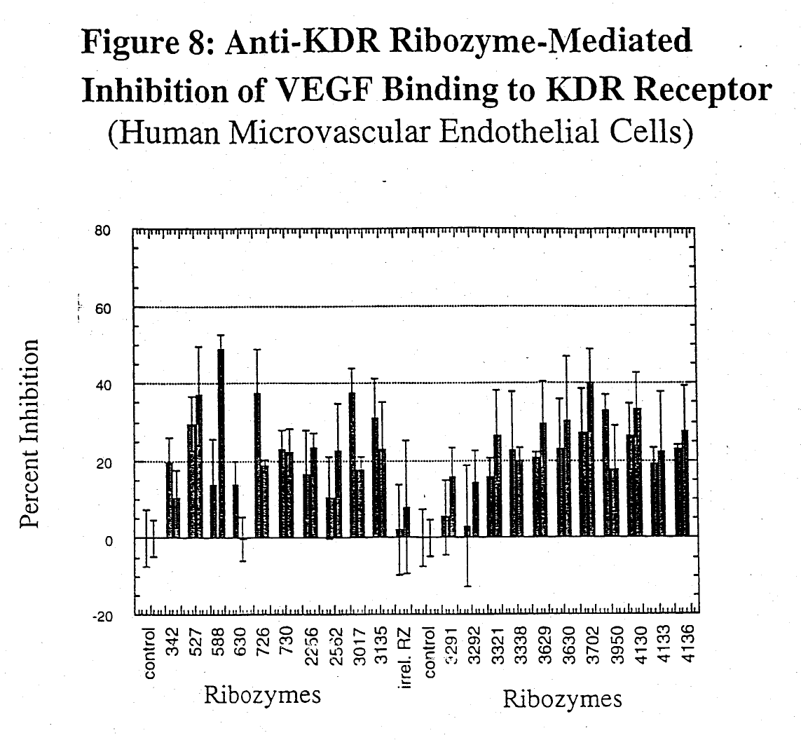

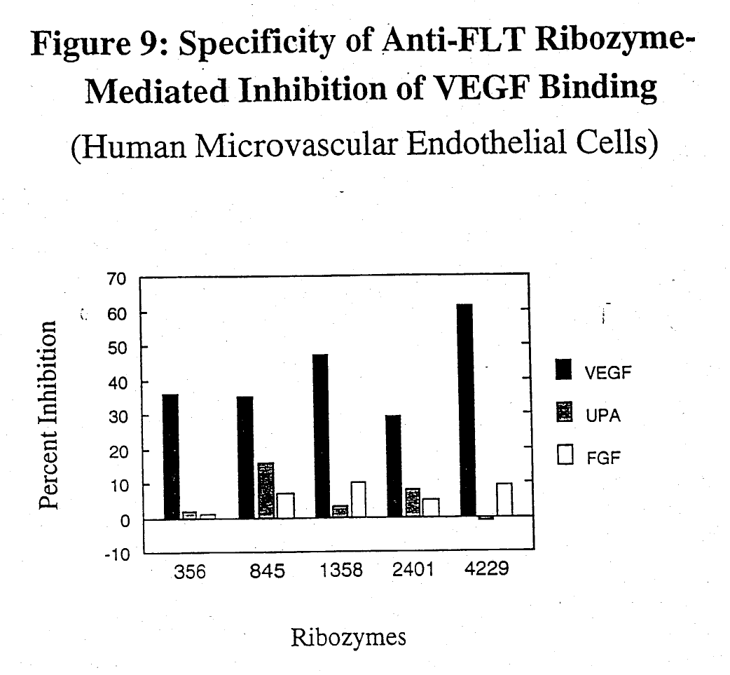

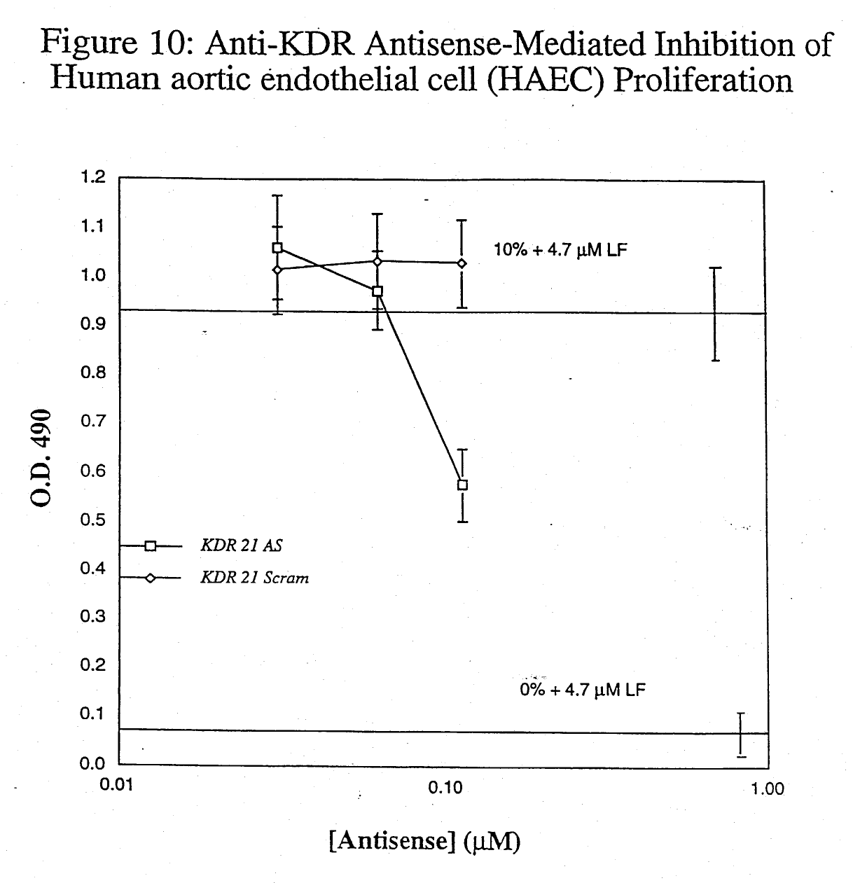

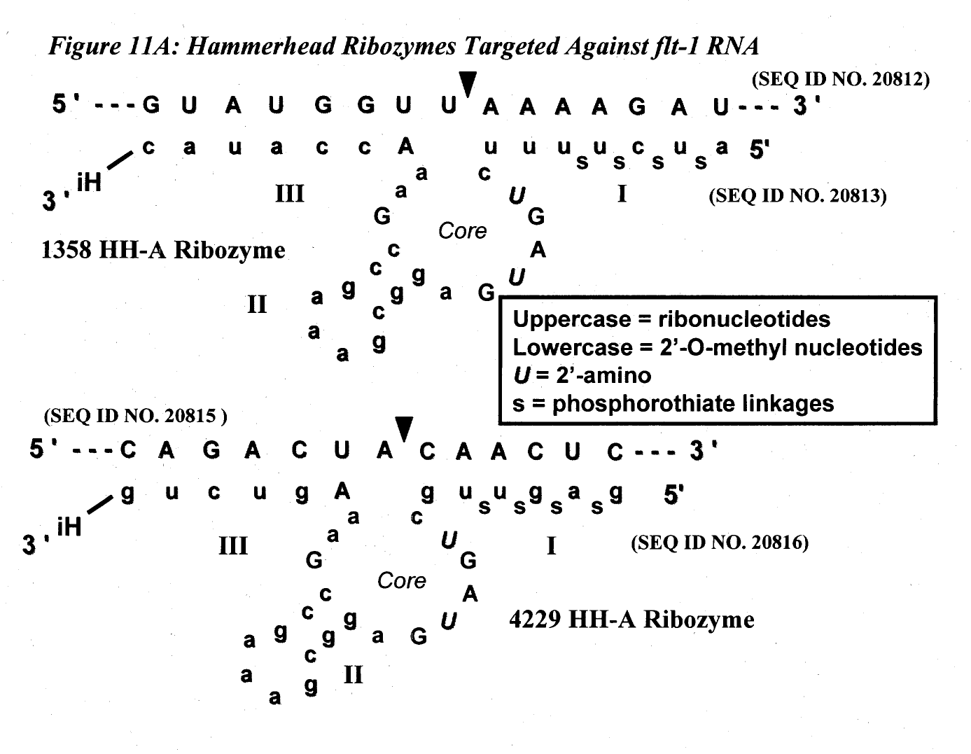

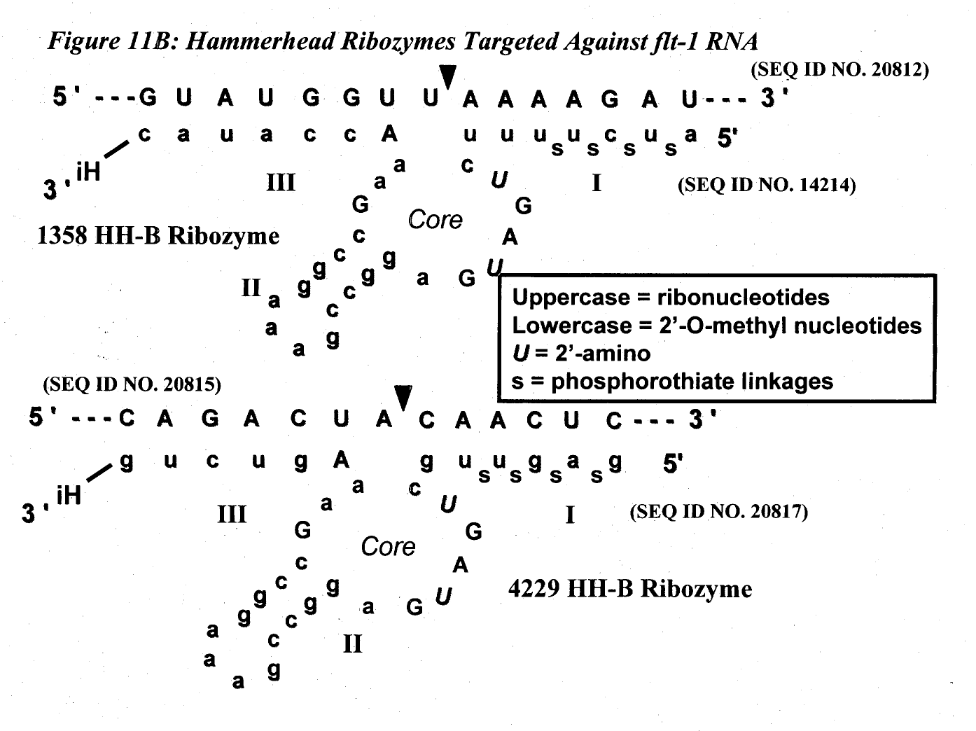

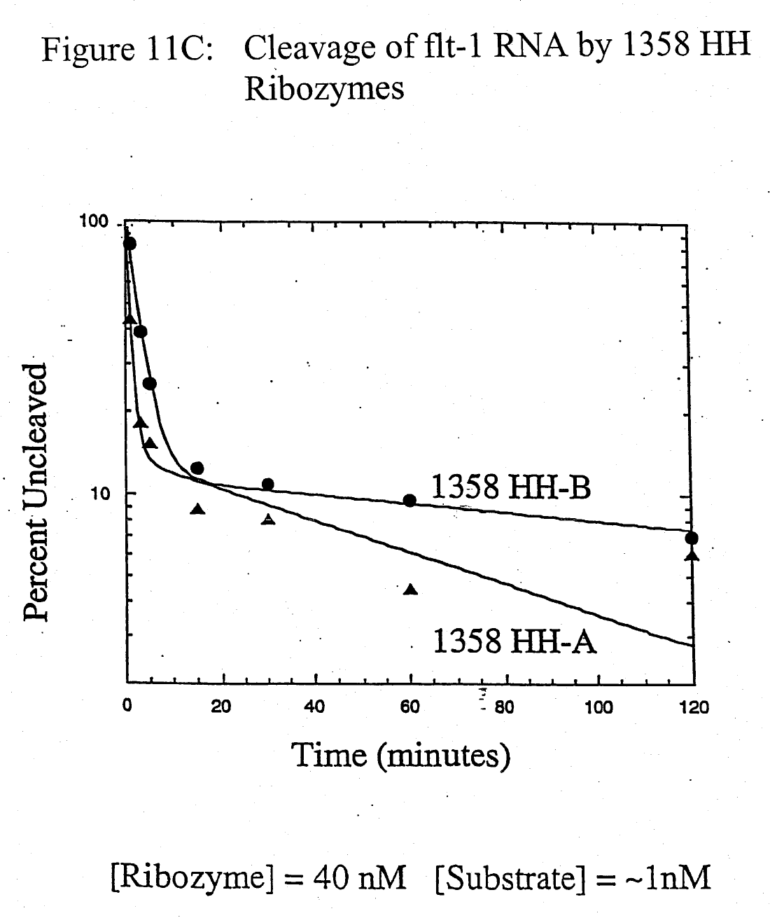

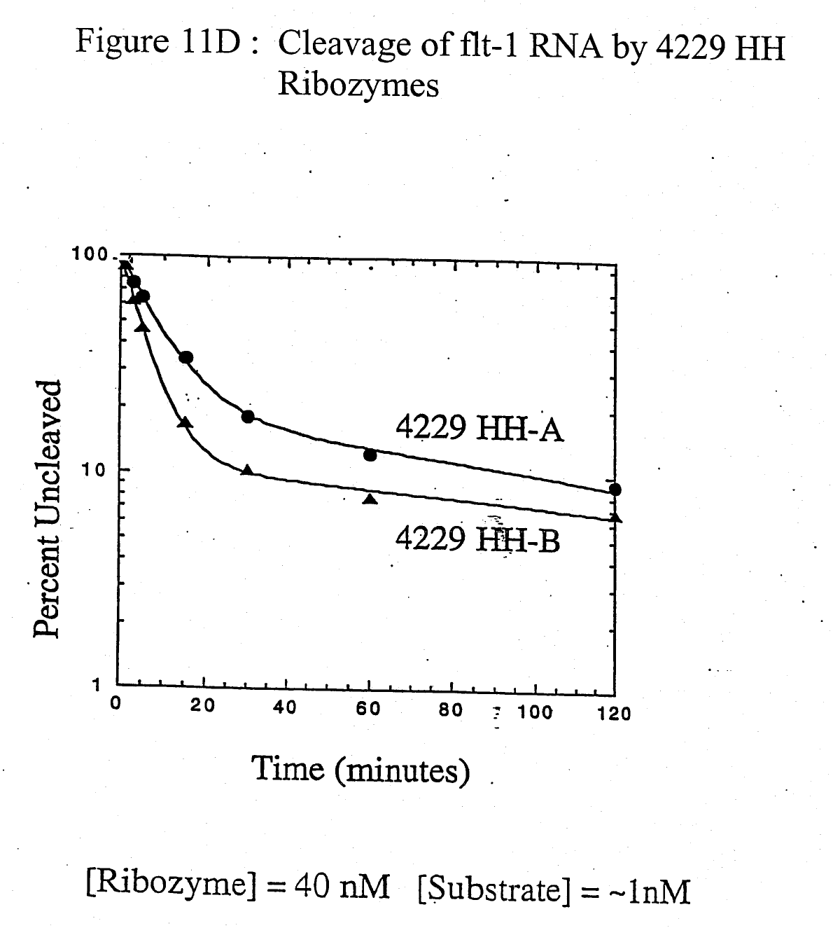

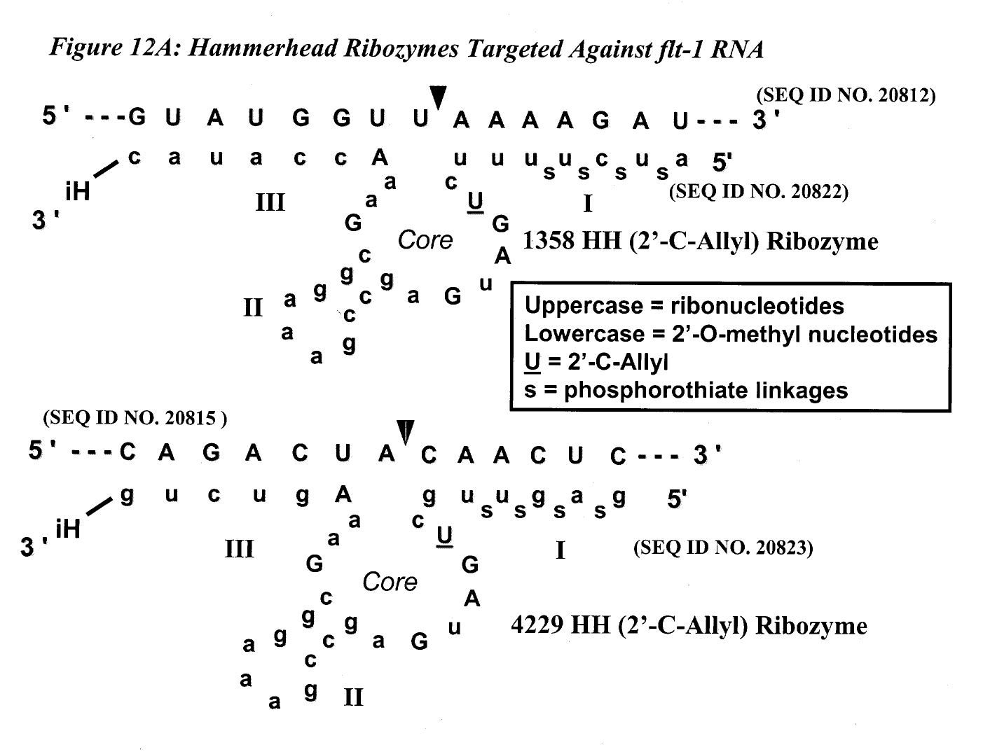

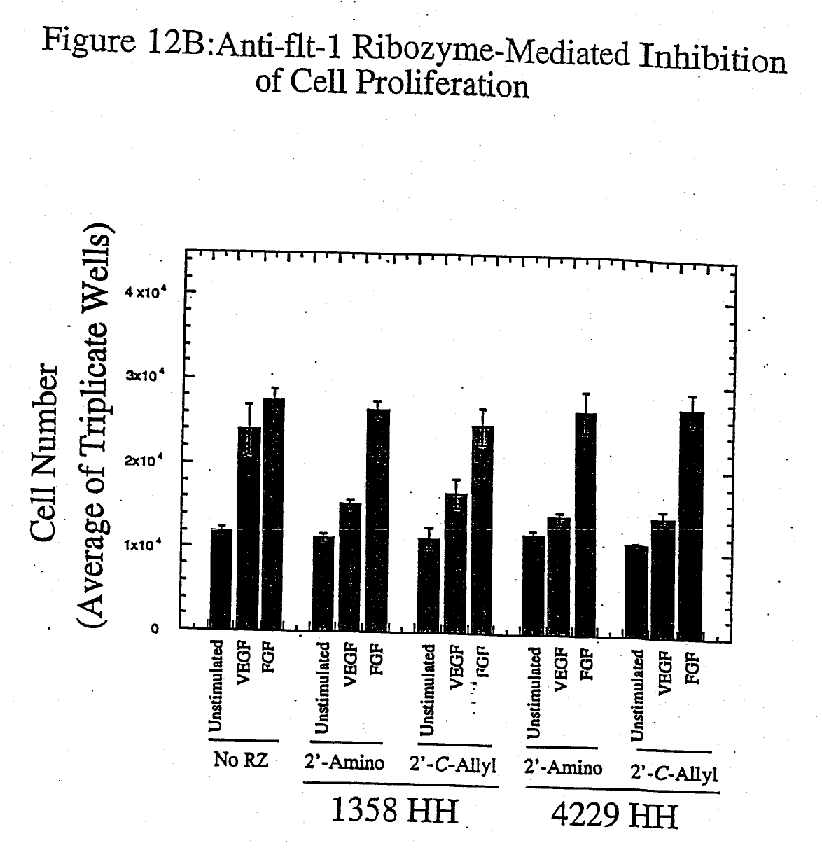

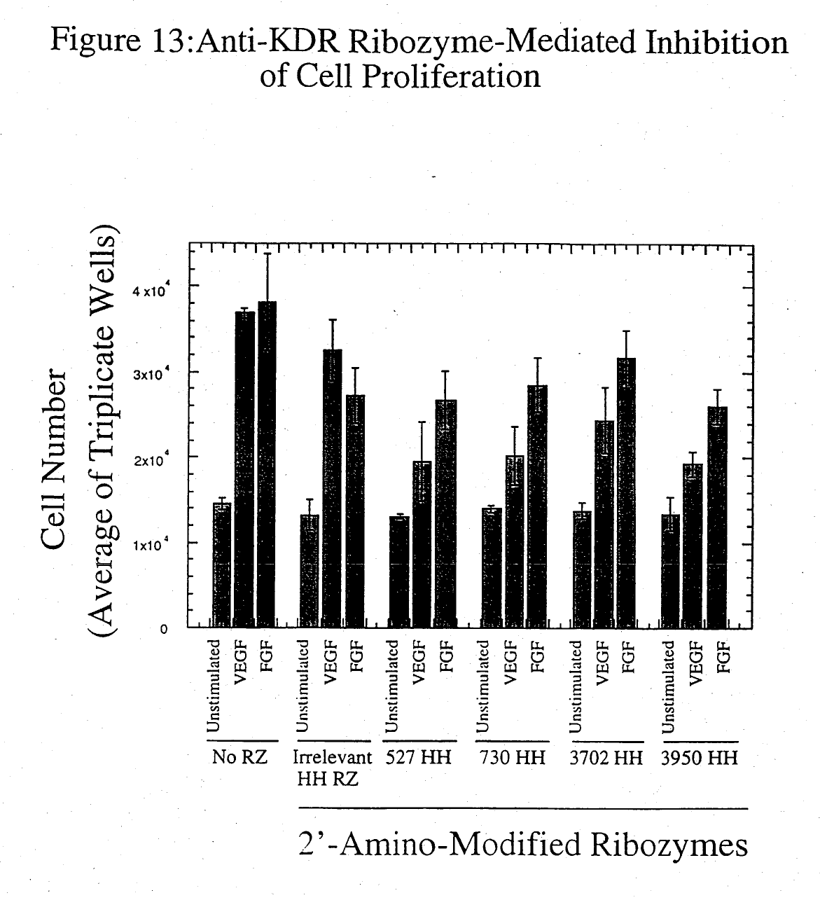

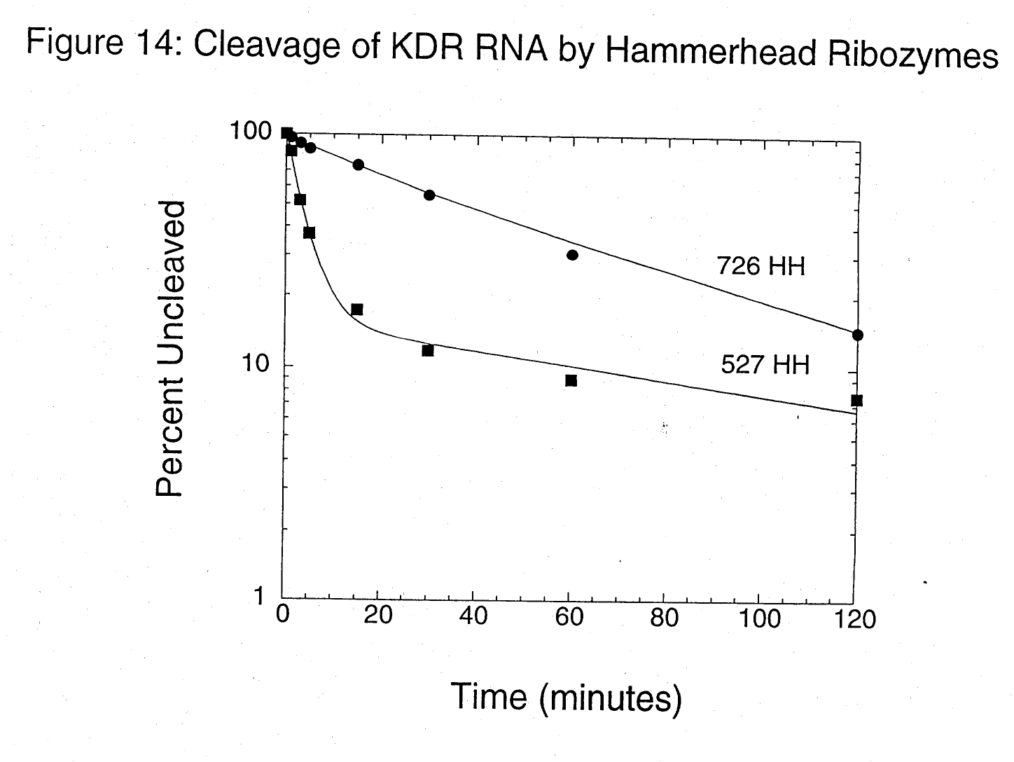

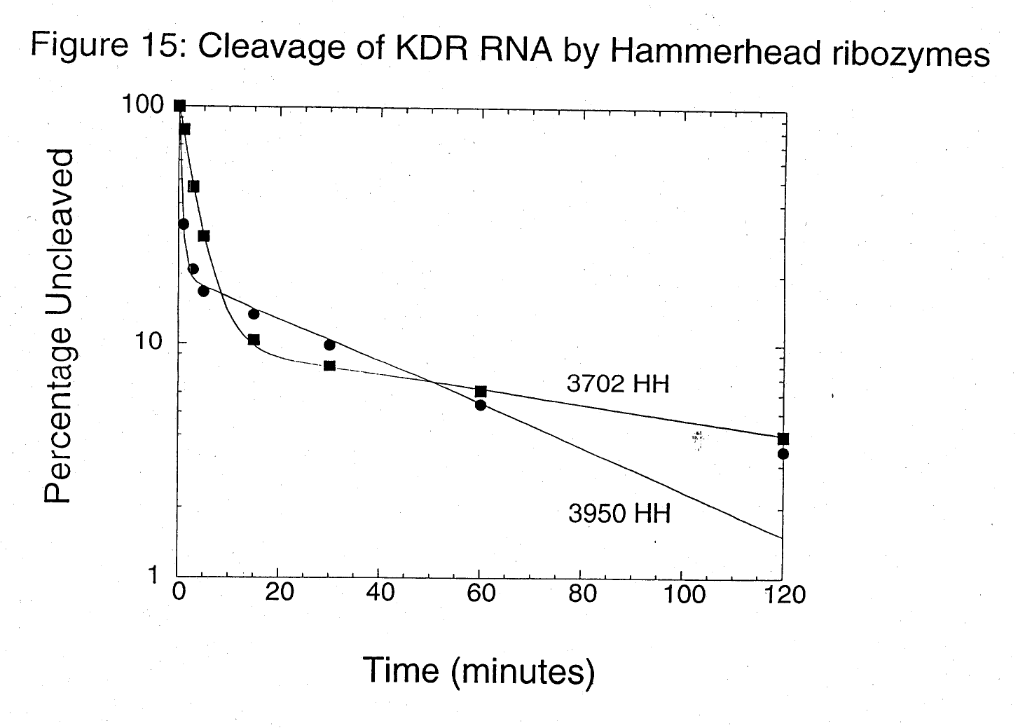

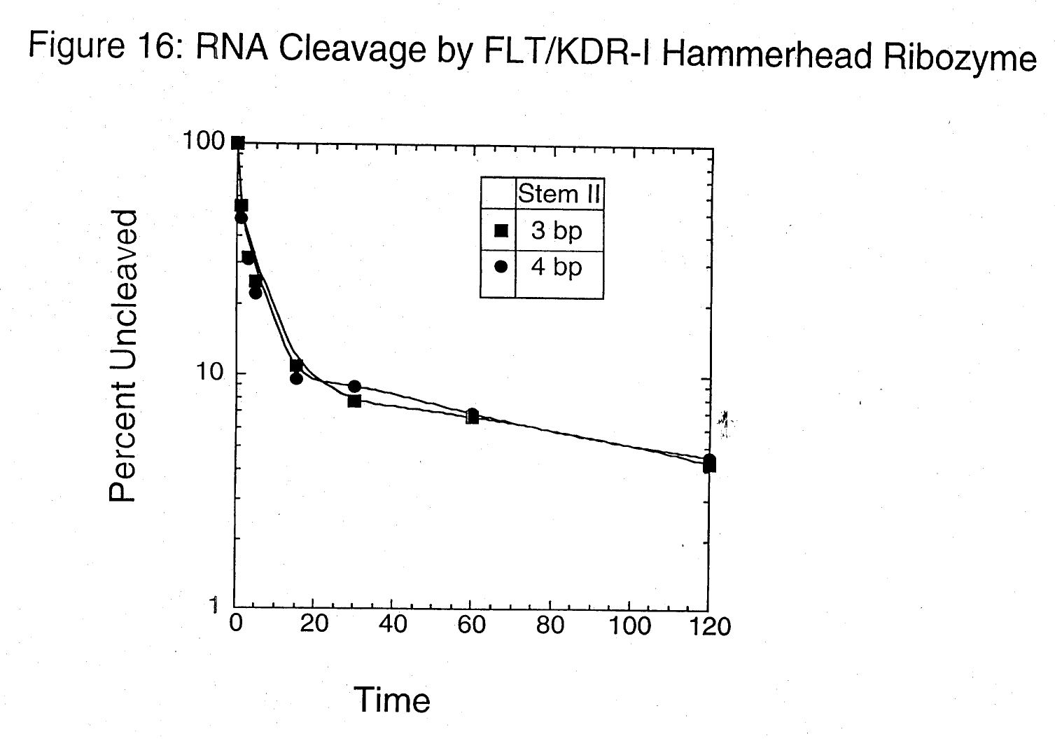

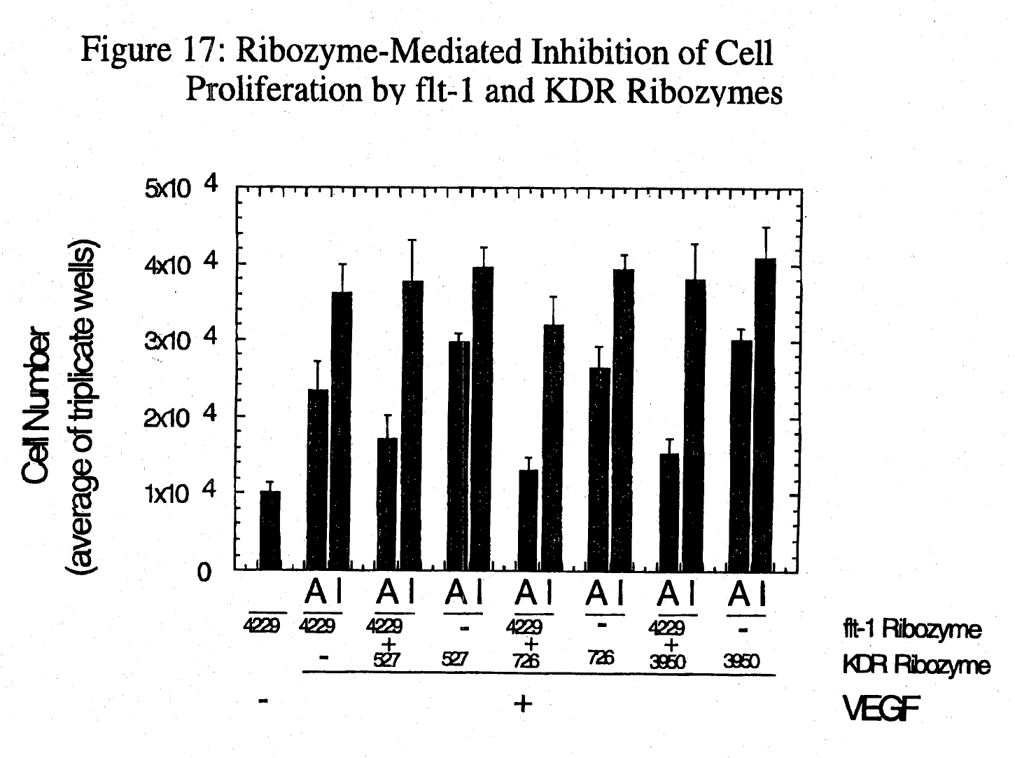

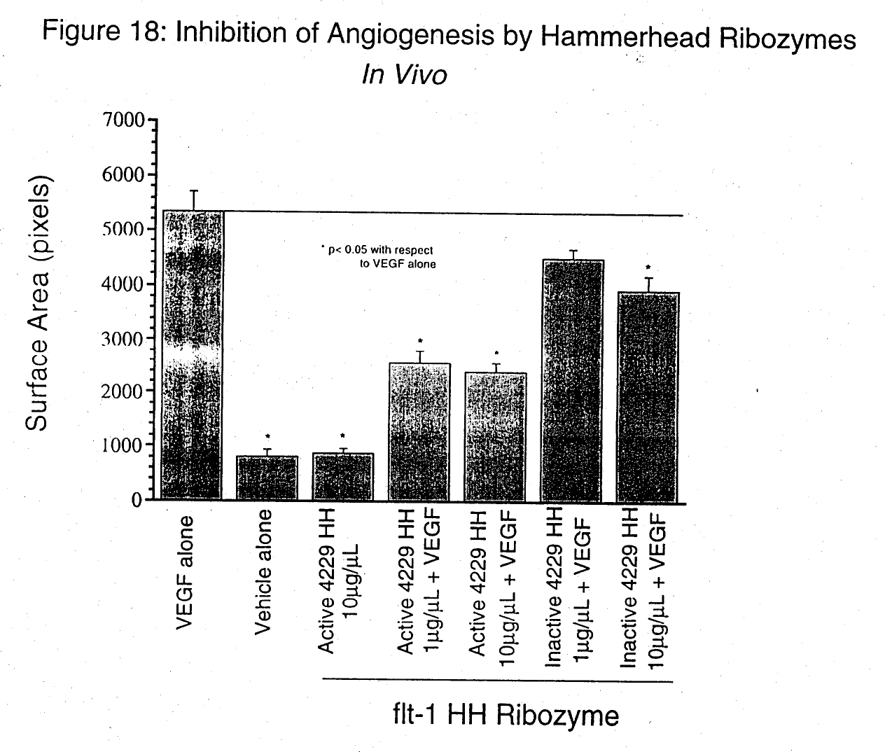

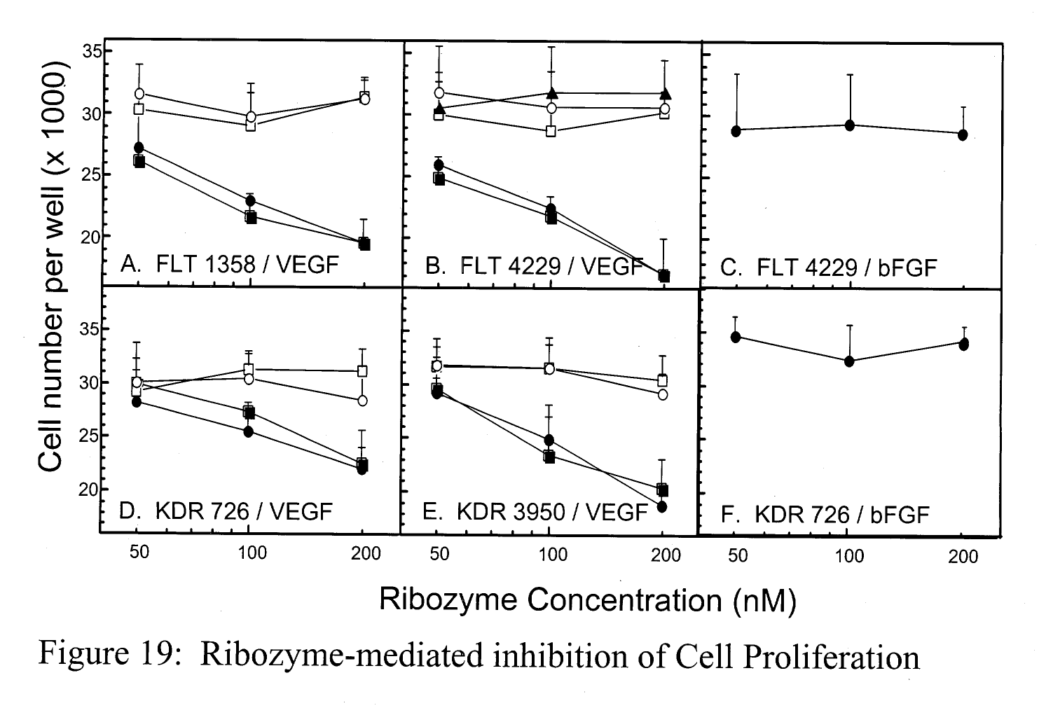

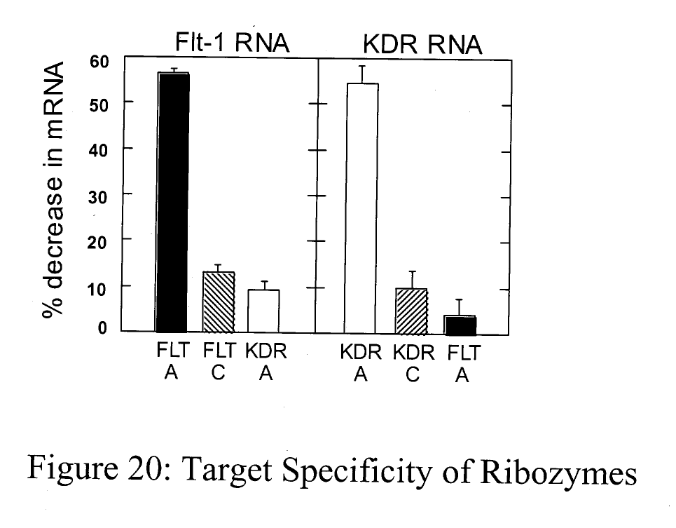

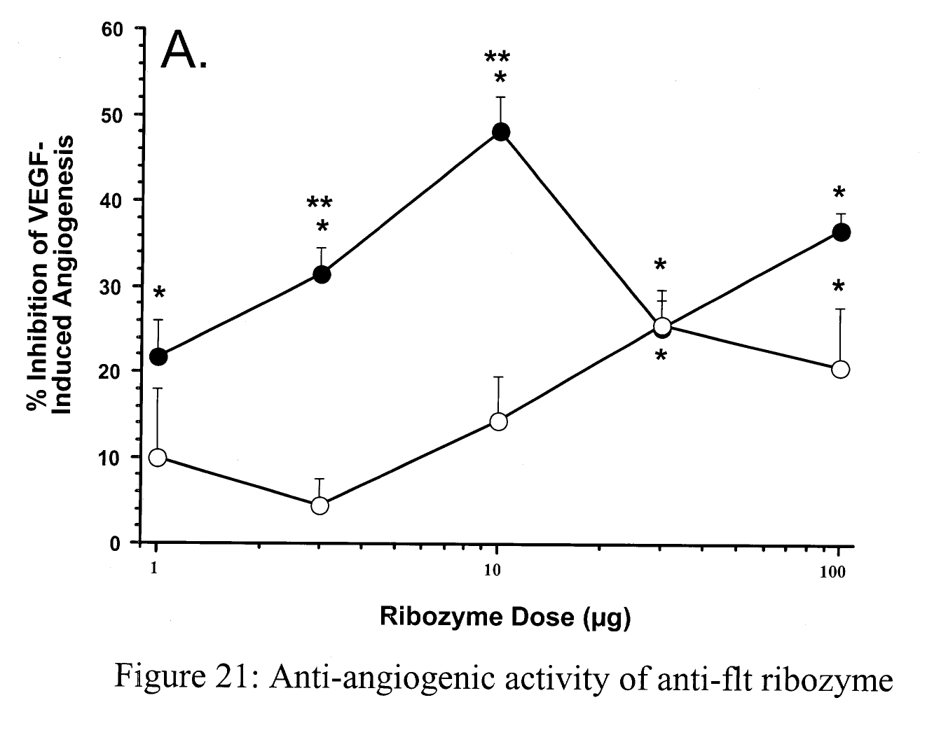

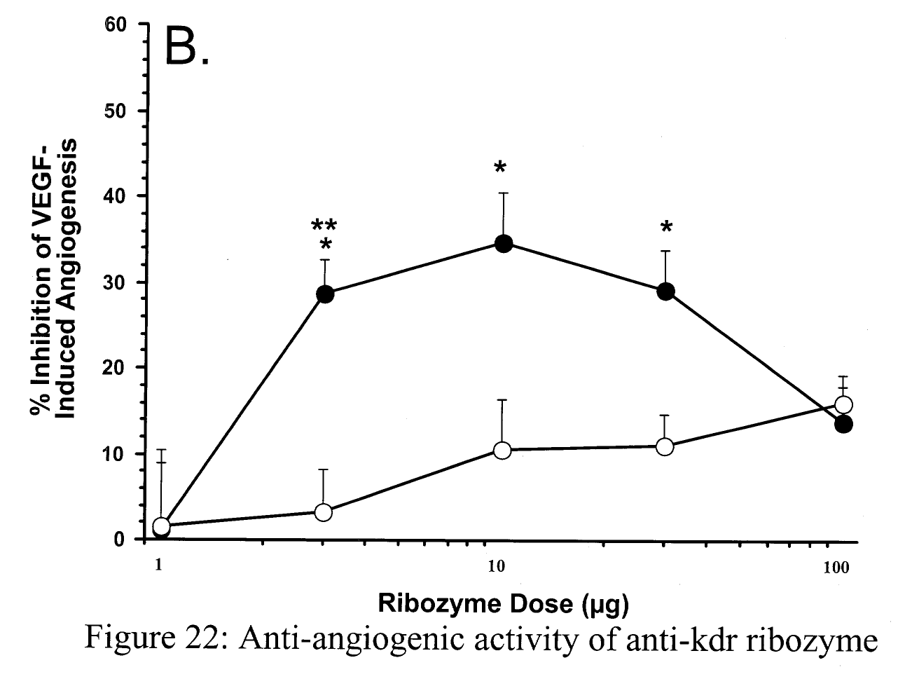

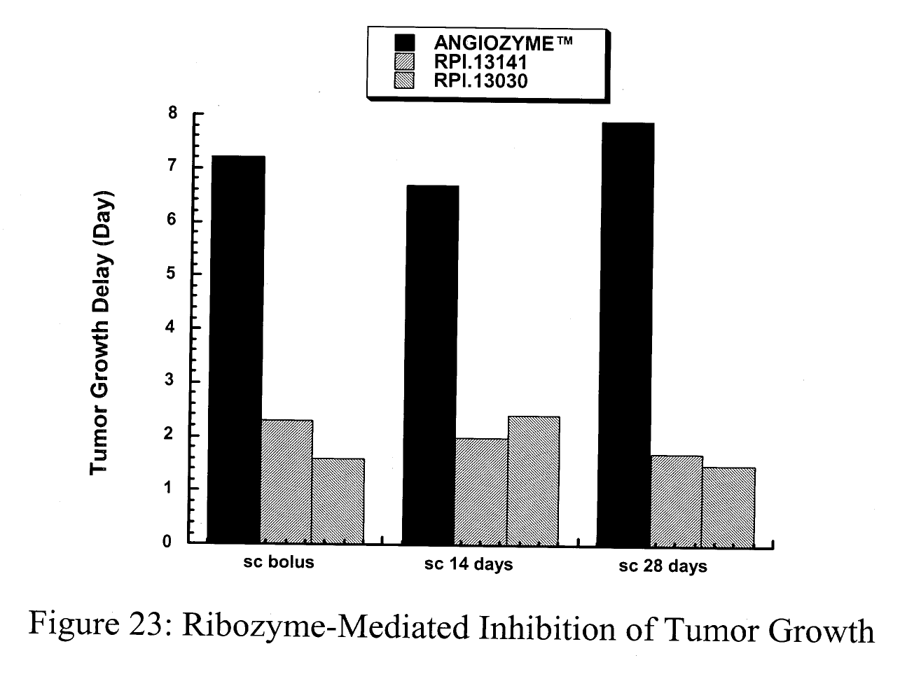

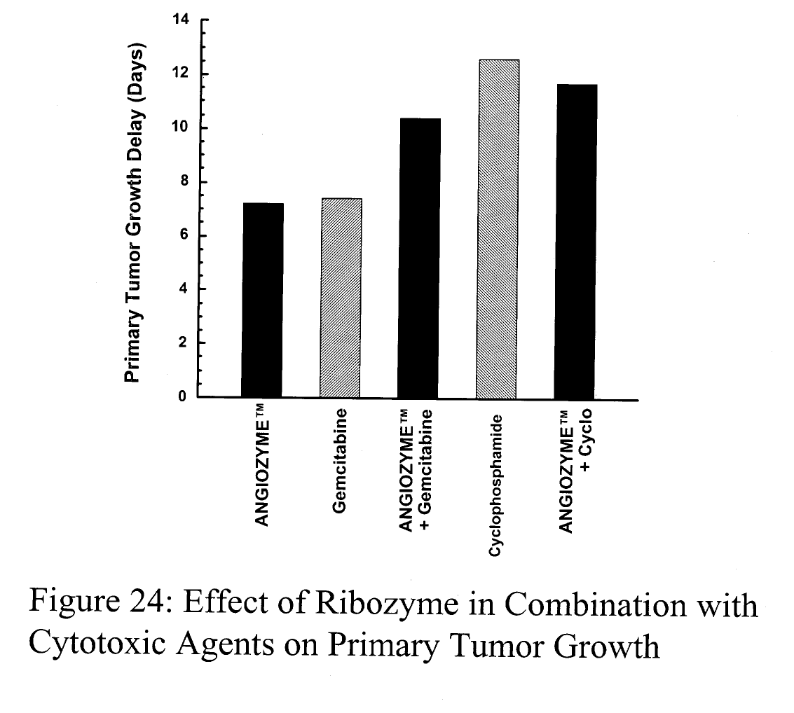

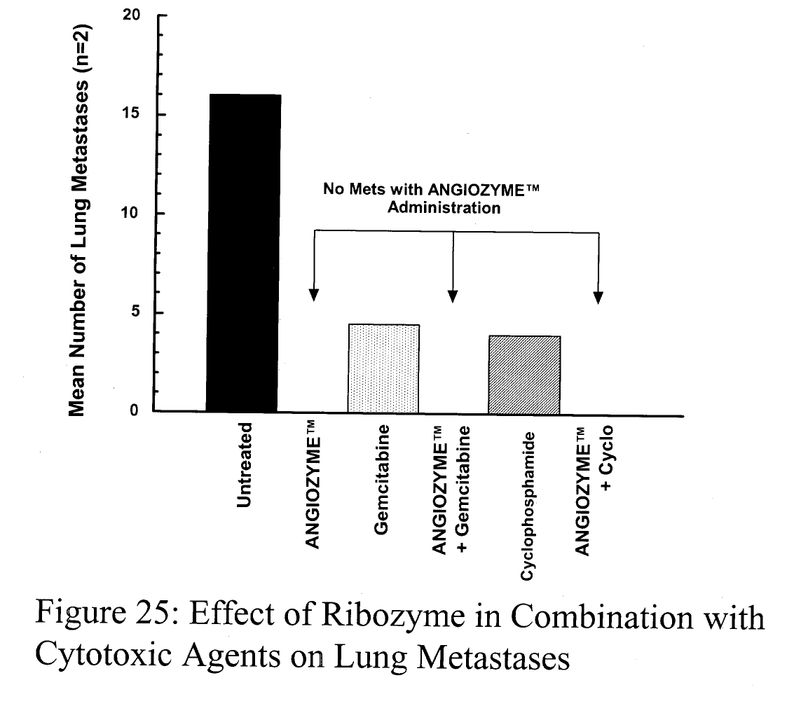

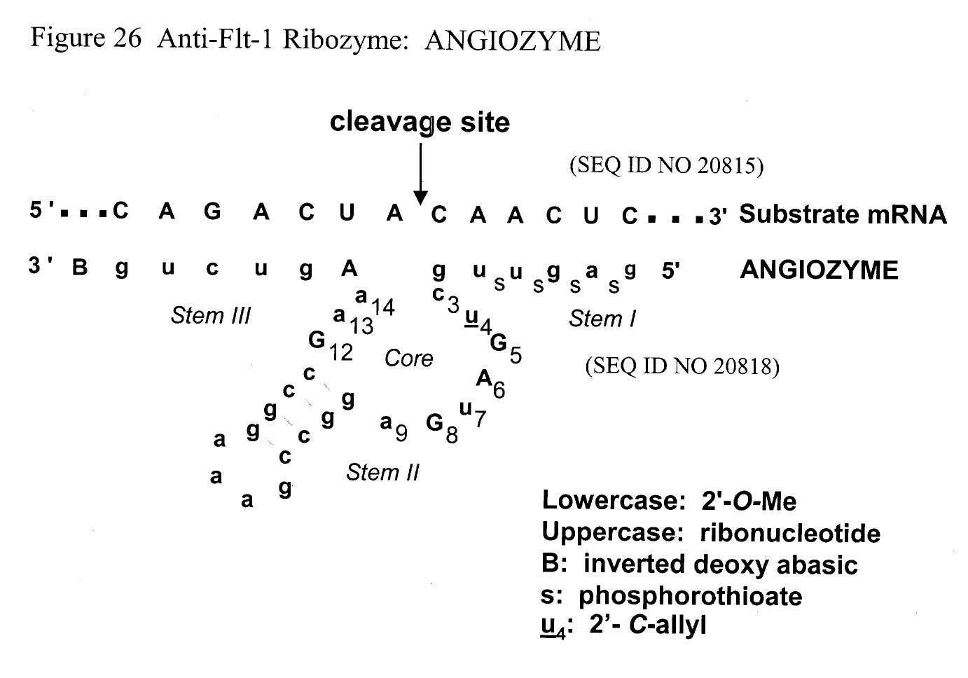

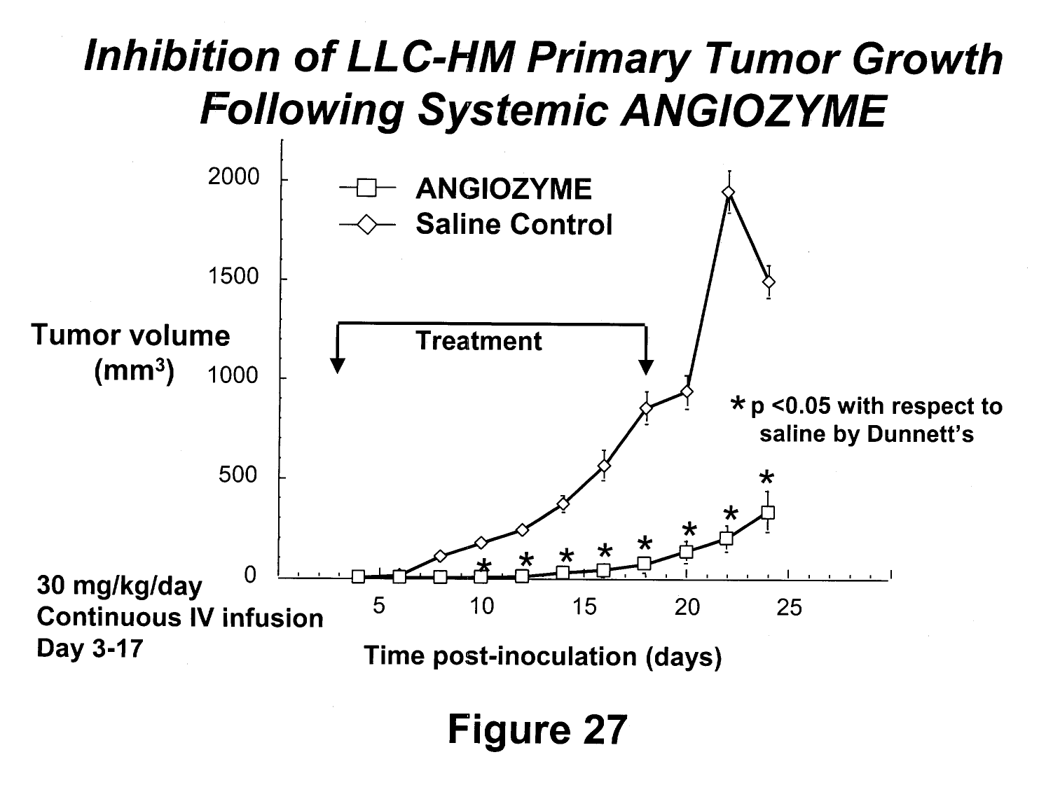

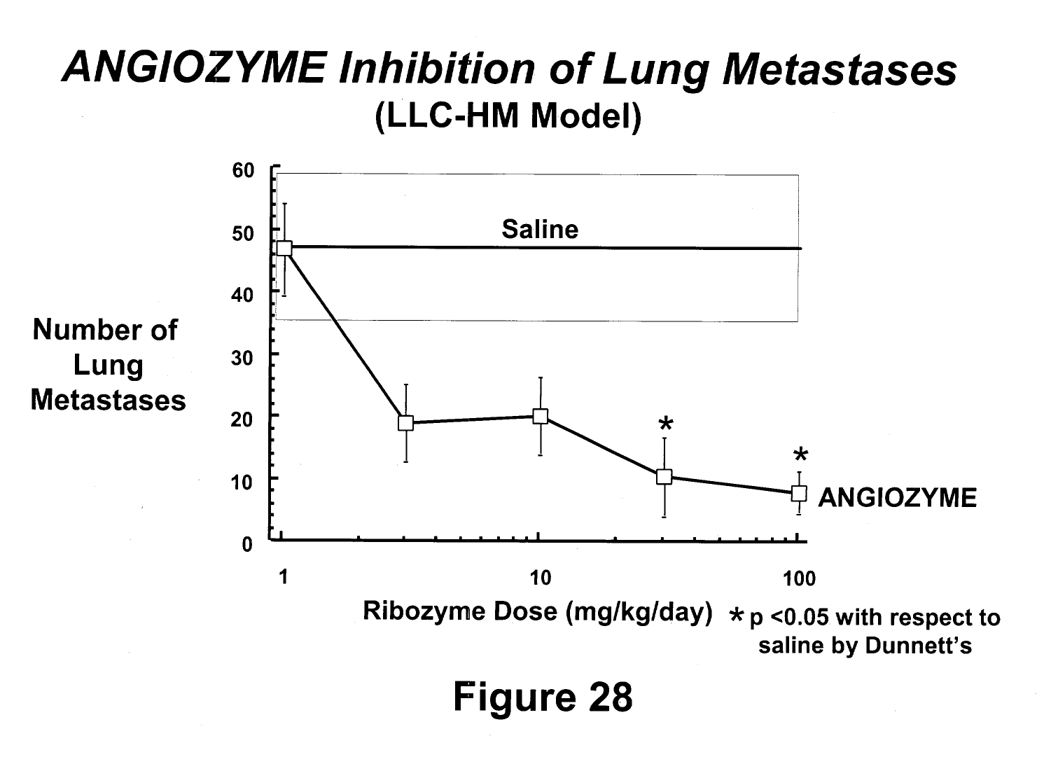

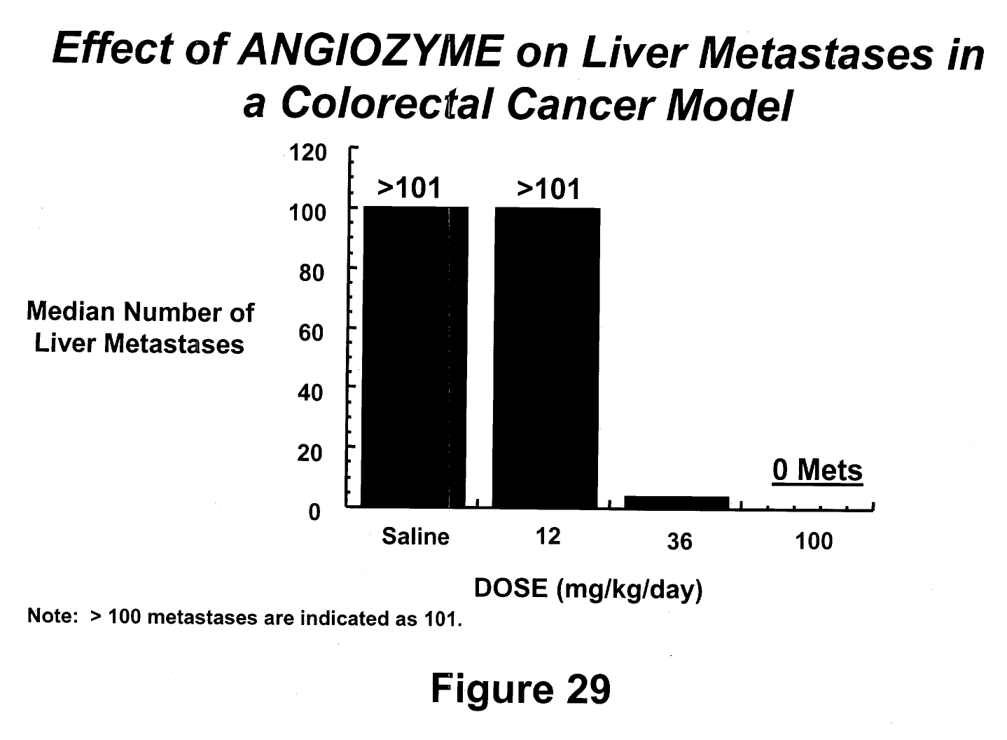

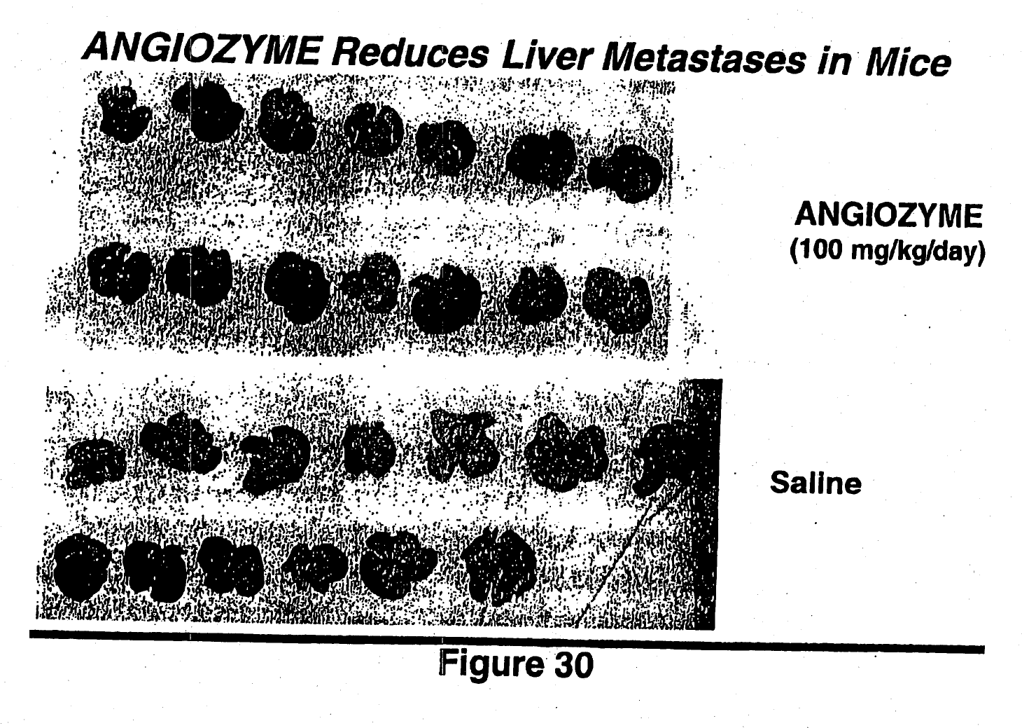

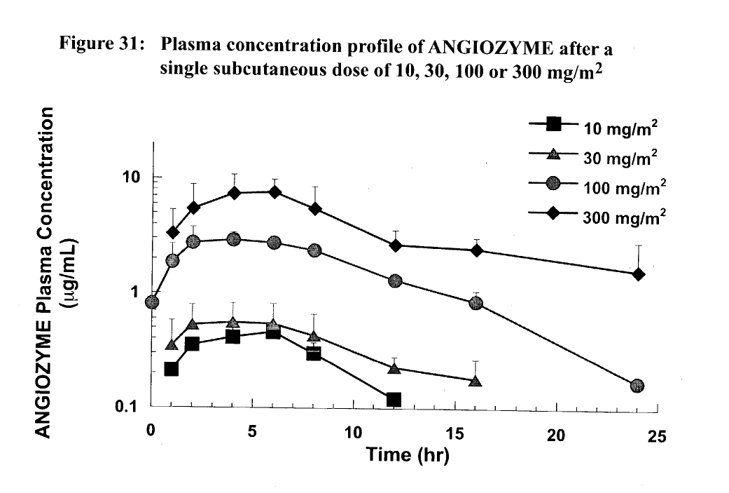

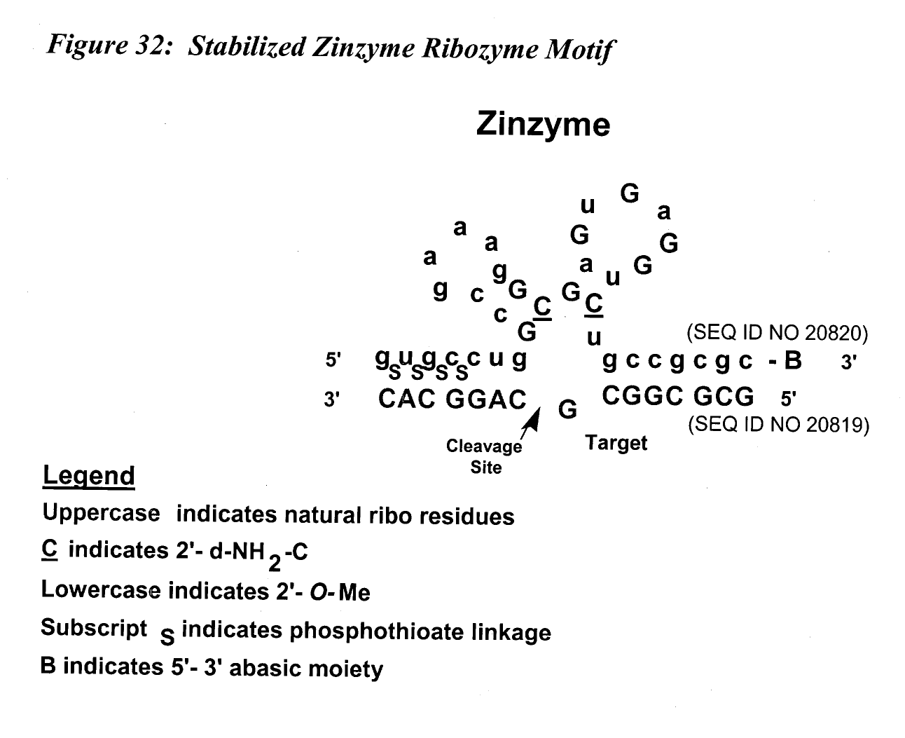

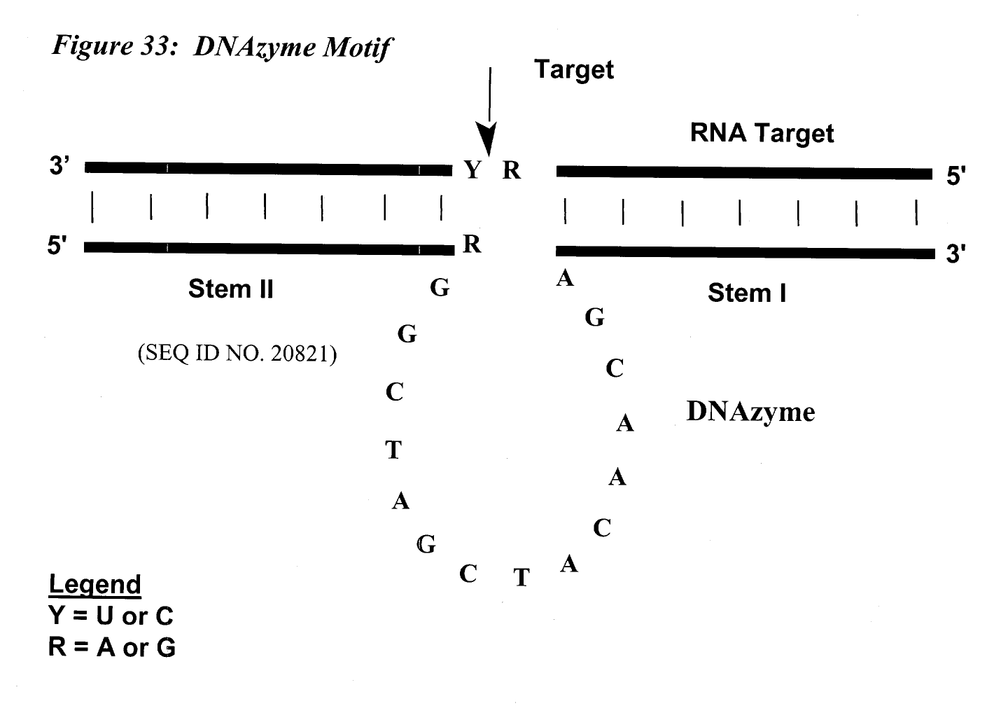

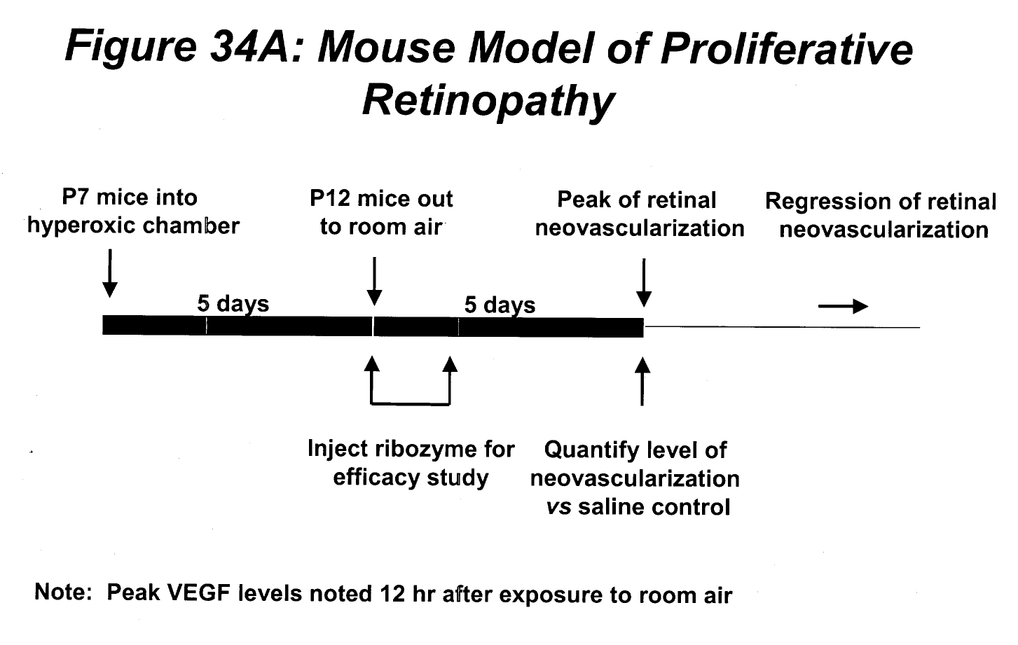

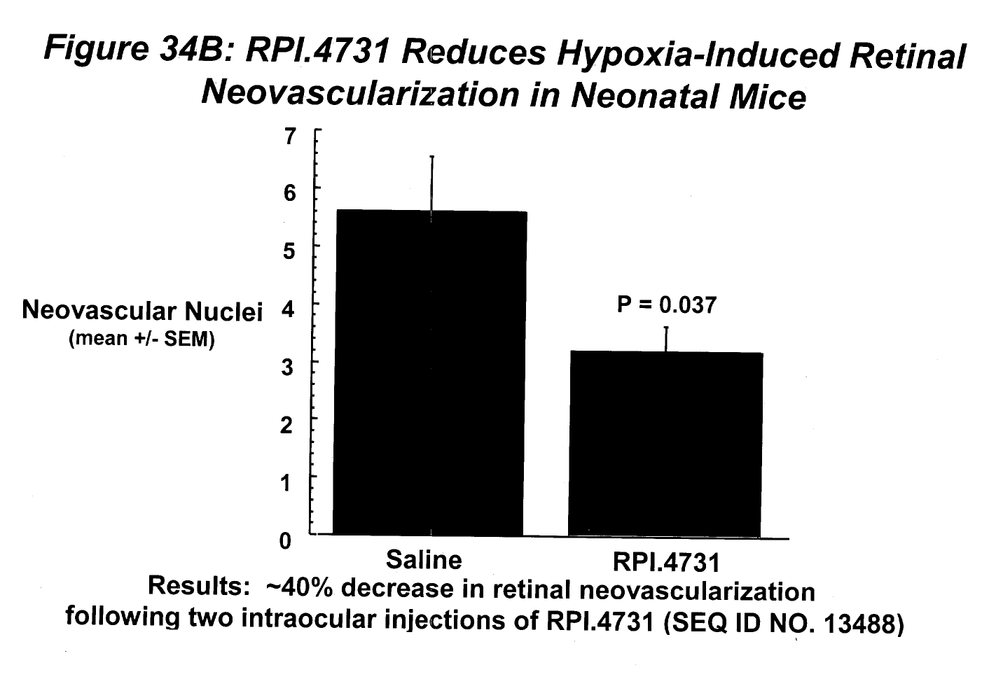

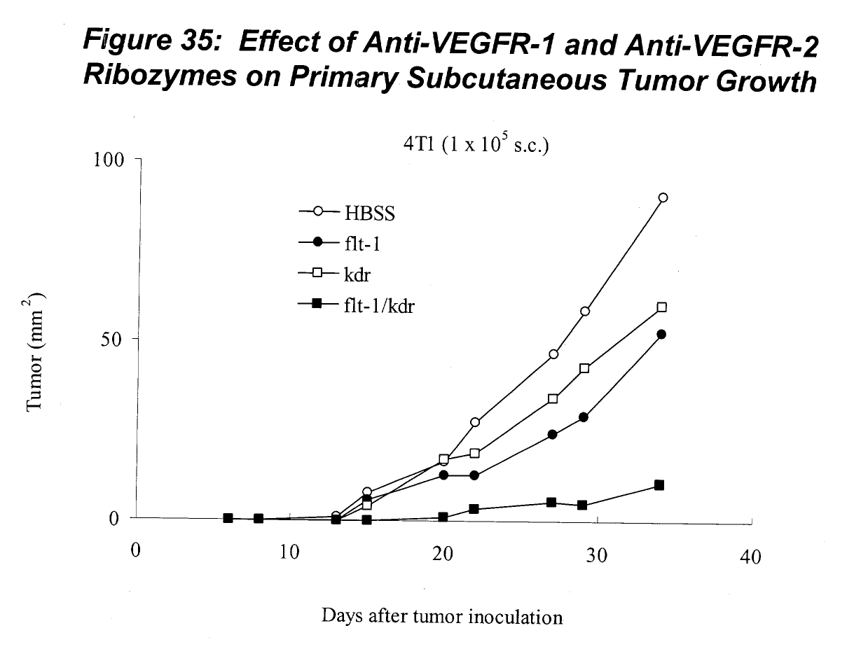

Group I Introns
Size: ˜200 to >1000 nucleotides.
Requires a U in the target sequence immediately 5′ of the cleavage
site.
Binds 4-6 nucleotides at 5′ side of cleavage site.
Over 75 known members of this class. Found in Tetrahymena
thermophila rRNA, fungal mitochondria, chloroplasts, phage T4,
blue-green algae, and others.
RNAseP RNA (M1 RNA)
Size: ˜290 to 400 nucleotides.
RNA portion of a ribonucleoprotein enzyme. Cleaves tRNA
precursors to form mature tRNA.
Roughly 10 known members of this group all are bacterial in origin.
Hammerhead Ribozyme
Size: ˜13 to 40 nucleotides.
Requires the target sequence UH immediately 5′ of the cleavage
site.
Binds a variable number of nucleotides on both sides of the
cleavage site.
14 known members of this class. Found in a number of plant
pathogens (virusoids) that use RNA as the infectious agent (FIGS. 1
and 2)
Hairpin Ribozyme
Size: ˜50 nucleotides.
Requires the target sequence GUC immediately 3′ of the cleavage
site.
Binds 4-6 nucleotides at 5′ side of the cleavage site and a variable
number to the 3′ side of the cleavage site.
Only 3 known member of this class. Found in three plant pathogen
(satellite RNAs of the tobacco ringspot virus, arabis mosaic virus
and chicory yellow mottle virus) which uses RNA as the infectious
agent (FIG. 3).
Hepatitis Delta Virus (HDV) Ribozyme
Size: 50-60 nucleotides (at present).
Sequence requirements not fully determined.
Binding sites and structural requirements not fully determined,
although no sequences 5′ of cleavage site are required.
Only 1 known member of this class. Found in human HDV (FIG. 4).
Neurospora VS RNA Ribozyme
Size: ˜144 nucleotides (at present)
Cleavage of target RNAs recently demonstrated.
Sequence requirements not fully determined.
Binding sites and structural requirements not fully determined. Only 1
known member of this class. Found in Neurospora VS RNA
(FIG. 5).

TABLE II
Human flt1 VEGF Receptor—Hammerhead Ribozyme and Substrate Sequence

| | | | | |
|---|---|---|---|---|
| | | Seq ID | | Seq ID |
| Pos | Substrate | No | HH Ribozyme | No |

| | | | | | |
|---|---|---|---|---|---|
| 10 | CGGACACUC CUCUCGGC | 1 | GCCGAGAG CUGAUGAGGCCGUUAGGCCCGAA AGUGUCCG | 9420 | |
| 13 | ACACUCCUC UCGGCUCC | 2 | GGAGCCGA CUGAUGAGGCCGUUAGGCCCGAA AGGAGUGU | 9421 | |
| 15 | ACUCCUCUC GGCUCCUC | 3 | GAGCAGCC CUGAUGAGGCCGUUAGGCCCGAA ACACGAGU | 9422 | |
| 20 | UCUCGGCUC CUCCCCGG | 4 | CCGGGGAG CUGAUGAGGCCGUUAGGCCCGAA AGCCCAGA | 9423 | |
| 23 | CGGCUCCUC CCCGGCAG | 5 | CUGCCGGG CUGAUGAGGCCGUUAGGCCCGAA AGGAGCCG | 9424 | |
| 43 | CGGCGGCUC GGAGCGGG | 6 | CCCGCUCC CUGAUGAGGCCGUUAGGCCCGAA AGCCGCCG | 9425 | |
| 54 | AGCGGGCUC CGGGGCUC | 7 | GAGCCCCG CUGAUGAGGCCGUUAGGCCCGAA AGCCCGCU | 9426 | |
| 62 | CCGCGGCUC GGGUGCAG | 8 | CUGCACCC CUGAUGAGGCCGUUAGGCCCGAA AGCCCCGG | 9427 | |
| 97 | GCCAGGAUU ACCCGCGG | 9 | CCCCGGGU CUGAUGAGGCCGUUAGGCCCGAA AUCCUCGC | 9428 | |
| 98 | CGAGGAUUA CCCCGGGA | 10 | UCCCCGGG CUGAUGAGGCCGUUAGGCCCGAA AAUCCUCC | 9429 | |
| 113 | GAAGUGGUU GUCUCCUG | 11 | CAGGAGAC CUGAUGAGGCCGUUAGGCCCGAA ACCACUUC | 9430 | |
| 116 | GUGGUUGUC UCCUGGCU | 12 | AGCCAGGA CUGAUGAGGCCGUUAGGCCGGAA ACAACCAC | 9431 | |
| 118 | GGUUGUCUC CUGGCUGG | 13 | CCAGCCAG CUGAUGAGGCCGUUAGGCCGGAA AGACAACC | 9432 | |
| 145 | CGGGCGCUC AGGGCGCG | 14 | CGCGCCCU CUGAUGAGGCCGUUAGGCCCGAA AGCGCCCG | 9433 | |
| 185 | GACGGACUC UGGCGGCC | 15 | GGCCGCCA CUGAUGAGGCCGUUAGGCCCGAA AGUCCGUC | 9434 | |
| 198 | GGCCGGGUC GUUGGCCG | 16 | CGGCCAAC CUGAUGAGGCCGUUAGGCCCGAA ACCCGGCC | 9435 | |
| 201 | CGGGUCGUU GGCCGGGG | 17 | CCCCGGCC CUGAUGAGGCCGUUAGGCCCGAA ACGACCCG | 9436 | |
| 240 | GGCCGCGUC GCGCUCAC | 18 | GUGAGCGC CUGAUGAGGCCGUUAGGCCCGAA ACGCGGCC | 9437 | |
| 246 | GUCGCGCUC ACCAUGGU | 19 | ACCAUGGU CUGAUGAGGCCGUUAGGCCCGAA AGCGCGAC | 9438 | |
| 255 | ACCAUGGUC AGCUACUG | 20 | CAGUAGCU CUGAUGAGGCCGUUAGGCCCGAA ACCAUGGU | 9439 | |
| 260 | GGUCAGCUA CUGGGACA | 21 | UGUCCCAG CUGAUGAGGCCGUUAGGCCCGAA AGCUGACC | 9440 | |
| 276 | ACCGGGGUC CUGCUGUG | 22 | CACAGCAG CUGAUGAGGCCGUUAGGCCCGAA ACCCCGGU | 9441 | |
| 294 | GCGCUGCUC AGCUGUCU | 23 | AGACAGCU CUGAUGAGGCCGUUAGGCCCGAA AGCAGCGC | 9442 | |
| 301 | UCAGCUGUC UGCUUCUC | 24 | GAGAAGCA CUGAUGAGGCCGUUAGGCCCGAA ACAGCUGA | 9443 | |
| 306 | UGUCUGCUU CUCACAGG | 25 | CCUGUGAG CUGAUGAGGCCGUUAGGCCCGAA AGCAGACA | 9444 | |
| 307 | GUCUGCUUC UCACAGGA | 26 | UCCUGUGA CUGAUGAGGCCGUUAGGCCCGAA AAGCAGAC | 9445 | |
| 309 | CUGCUUCUC ACAGGAUC | 27 | GAUCCUGU CUGAUGAGGCCGUUAGGCCCGAA AGAAGCAG | 9446 | |
| 317 | CACAGGAUC UAGUUCAG | 28 | CUGAACUA CUGAUGAGGCCGUUAGGCCCGAA AUCCUGUG | 9447 | |
| 319 | CAGGAUCUA GUUCAGGU | 29 | ACCUGAAC CUGAUGAGGCCGUUAGGCCCGAA AGAUCCUG | 9448 | |
| 322 | GAUCUAGUU CAGGUUCA | 30 | UGAACCUG CUGAUGAGGCCGUUAGGCCCGAA ACUAGAUC | 9449 | |
| 323 | AUCUAGUUC AGGUUCAA | 31 | UUGAACCU CUGAUGAGGCCGUUAGGCCCGAA AACUAGAU | 9450 | |
| 328 | GUUCAGGUU CAAAAUUA | 32 | UAAUUUUG CUGAUGAGGCCGUUAGGCCCGAA ACCUGAAC | 9451 | |
| 329 | UUCAGGUUC AAAAUUAA | 33 | UUAAUUUU CUGAUGAGGCCGUUAGGCCCGAA AACCUGAA | 9452 | |
| 335 | UUCAAAAUU AAAAGAUC | 34 | GAUCUUUU CUGAUGAGGCCGUUAGGCCCGAA AUUUUGAA | 9453 | |
| 336 | UCAAAAUUA AAAGAUCC | 35 | GGAUCUUU CUGAUGAGGCCGUUAGGCCCGAA AAUUUUGA | 9454 | |
| 343 | UAAAAGAUC CUGAACUG | 36 | CAGUUCAG CUGAUGAGGCCGUUAGGCCCGAA AUCUUUUA | 9455 | |
| 355 | AACUGAGUU UAAAAGGC | 37 | GCCUUUUA CUGAUGAGGCCGUUAGGCCCGAA ACUCAGUU | 9456 | |
| 356 | ACUGAGUUU AAAAGGCA | 38 | UGCCUUUU CUGAUGAGGCCGUUAGGCCCGAA AACUCAGU | 9457 | |
| 357 | CUGAGUUUA AAAGGCAC | 39 | GUGCCUUU CUGAUGAGGCCGUUAGGCCCGAA AAACUCAG | 9458 | |
| 375 | CAGCACAUC AUGCAAGC | 40 | GCUUGCAU CUGAUGAGGCCGUUAGGCCCGAA AUGUGCUG | 9459 | |
| 400 | CACUCCAUC UCCAAUGC | 41 | GCAUUGGA CUGAUGAGGCCGUUAGGCCCGAA AUGCAGUG | 9460 | |
| 402 | CUGCAUCUC CAAUGCAG | 42 | CUGCAUUG CUGAUGAGGCCGUUAGGCCCGAA AGAUGCAG | 9461 | |
| 427 | CAGCCCAUA AAUGGUCU | 43 | AGACCAUU CUGAUGAGGCCGUUAGGCCCGAA AUGGGCUG | 9462 | |
| 434 | UAAAUGGUC UUUGCCUG | 44 | CAGGCAAA CUGAUGAGGCCGUUAGGCCCGAA ACCAUUUA | 9463 | |
| 436 | AAUGGUCUU UGCCUGAA | 45 | UUCAGGCA CUGAUCAGGCCGUUAGGCCCGAA AGACCAUU | 9464 | |
| 437 | AUGGUCUUU GCCUGAAA | 46 | UUUCAGGC CUGAUGAGGCCGUUAGGCCCGAA AAGACCAU | 9465 | |
| 454 | UGGUGAGUA AGGAAAGC | 47 | CCUUUCCU CUGAUGAGGCCGUUAGGCCCGAA ACUCACCA | 9466 | |
| 477 | CUGACCAUA ACUAAAUC | 48 | GAUUUAGU CUGAUGAGGCCGUUAGGCCCGAA AUCCUCAG | 9467 | |
| 481 | GCAUAACUA AAUCUGCC | 49 | GGCAGAUU CUGAUGAGGCCGUUAGGCCCGAA AGUUAUGC | 9468 | |
| 485 | AACUAAAUC UGCCUGUG | 50 | CACAGGCA CUGAUGAGGCCGUUAGGCCCGAA AUUUAGUU | 9469 | |
| 512 | CAAACAAUU CUGCAGUA | 51 | UACUGCAG CUCAUGAGGCCGUUAGGCCCGAA AUUGUUUG | 9470 | |
| 513 | AAACAAUUC UGCAGUAC | 52 | GUACUCCA CUGAUGAGGCCGUUAGGCCCGAA AAUUGUUU | 9471 | |
| 520 | UCUGCAGUA CUUUAACC | 53 | GGUUAAAG CUGAUGAGGCCGUUAGGCCCGAA ACUGCAGA | 9472 | |
| 523 | GCAGUACUU UAACCUUG | 54 | CAAGGUUA CUGAUGAGGCCGUUAGGCCCGAA AGUACUGC | 9473 | |
| 524 | CAGUACUUU AACCUUGA | 55 | UCAAGGUU CUGAUGAGGCCGUUAGGCCCGAA AAGUACUG | 9474 | |
| 525 | AGUACUUUA ACCUUGAA | 56 | UUCAAGGU CUGAUGAGGCCGUUAGGCCCGAA AAAGUACU | 9475 | |
| 530 | UUUAACCUU GAACACAG | 57 | CUGUGUUC CUGAUGAGGCCGUUAGGCCCGAA AGGUUAAA | 9476 | |
| 541 | ACACAUCUC AAGCAAAC | 58 | GUUUGCUU CUGAUGAGGCCGUUAGGCCCGAA AGCUCUGU | 9477 | |
| 560 | CACUGGCUU CUACAGCU | 59 | ACCUGUAG CUGAUGAGGCCGUUAGGCCCGAA AGCCAGUG | 9478 | |
| 561 | ACUGGCUUC UACAGCUG | 60 | CAGCUGUA CUGAUGAGGCCGUUAGGCCCGAA AAGCCAGU | 9479 | |
| 563 | UGGCUUCUA CAGCUGCA | 61 | UGCAGCUG CUGAUGAGGCCGUUAGGCCCGAA AGAAGCCA | 9480 | |
| 575 | CUGCAAAUA UCUAGCUG | 62 | CAGCUAGA CUGAUGAGGCCGUUAGGCCCGAA AUUUGCAG | 9481 | |
| 577 | GCAAAUAUC UAGCUGUA | 63 | UACAGCUA CUGAUGAGGCCGUUAGGCCCGAA AUAUUUGC | 9482 | |
| 579 | AAAUAUCUA GCUGUACC | 64 | GGUACAGC CUGAUGAGGCCGUUAGGCCCGAA AGAUAUUU | 9483 | |
| 585 | CUAGCUGUA CCUACUUC | 65 | GAAGUAGG CUGAUGAGGCCGUUAGGCCCGAA ACACCUAG | 9484 | |
| 589 | CUGUACCUA CUUCAAAG | 66 | CUUUGAAG CUGAUGAGGCCGUUAGGCCCGAA AGGUACAG | 9485 | |
| 592 | UACCUACUU CAAAGAAG | 67 | CUUCUUUG CUGAUGAGGCCGUUAGGCCCGAA AGUAGGUA | 9486 | |
| 593 | ACCUACUUC AAAGAAGA | 68 | UCUUCUUU CUGAUGAGGCCGUUAGGCCCGAA AAGUAGGU | 9487 | |
| 614 | AACAGAAUC UGCAAUCU | 69 | AGAUUGCA CUGAUGAGGCCGUUAGGCCCGAA AUUCUGUU | 9488 | |
| 621 | UCUGCAAUC UAUAUAUU | 70 | AAUAUAUA CUGAUGAGGCCGUUAGGCCCGAA AUUGCAGA | 9489 | |
| 623 | UGCAAUCUA UAUAUUUA | 71 | UAAAUAUA CUGAUGAGGCCGUUAGGCCCGAA AGAUUGCA | 9490 | |
| 625 | CAAUCUAUA UAUUUAUU | 72 | AAUAAAUA CUGAUGAGGCCGUUAGGCCCGAA AUAGAUUG | 9491 | |
| 627 | AUCUAUAUA UUUAUUAG | 73 | CUAAUAAA CUGAUGAGGCCGUUAGGCCCGAA AUAUAGAU | 9492 | |
| 629 | CUAUAUAUU UAUUAGUG | 74 | CACUAAUA CUGAUGAGGCCGUUAGGCCCGAA AUAUAUAG | 9493 | |
| 630 | UAUAUAUUU AUUAGUGA | 75 | UCACUAAU CUGAUGAGGCCGUUAGGCCCGAA AAUAUAUA | 9494 | |
| 631 | AUAUAUUUA UUAGUGAU | 76 | AUCACUAA CUGAUGAGGCCGUUAGGCCCGAA AAAUAUAU | 9495 | |
| 633 | AUAUUUAUU AGUGAUAC | 77 | GUAUCACU CUGAUGAGGCCGUUAGGCCCGAA AUAAAUAU | 9496 | |
| 634 | UAUUUAUUA GUGAUACA | 78 | UGUAUCAC CUGAUGAGGCCGUUAGGCCCGAA AAUAAAUA | 9497 | |
| 640 | UUAGUGAUA CAGGUAGA | 79 | UCUACCUG CUGAUGAGGCCGUUAGGCCCGAA AUCACUAA | 9498 | |
| 646 | AUACAGGUA GACCUUUC | 80 | GAAAGGUC CUGAUGAGGCCGUUAGGCCCGAA ACCUGUAU | 9499 | |
| 652 | GUAGACCUU UCCUAGAG | 81 | CUCUACGA CUGAUGAGGCCGUUAGGCCCGAA AGGUCUAC | 9500 | |
| 653 | UAGACCUUU CGUAGAGA | 82 | UCUCUACG CUGAUGAGGCCGUUAGGCCCGAA AAGGUCUA | 9501 | |
| 654 | AGACCUUUC GUAGAGAU | 83 | AUCUCUAC CUGAUGAGGCCGUUAGGCCCGAA AAAGGUCU | 9502 | |
| 657 | CCUUUCGUA GAGAUGUA | 84 | UACAUCUC CUGAUGAGGCCGUUAGGCCCGAA ACGAAAGG | 9503 | |
| 665 | AGAGAUGUA CAGUGAAA | 85 | UUUCACUG CUGAUGAGGCCGUUAGGCCCGAA ACAUCUCU | 9504 | |
| 675 | AGUGAAAUC CCCGAAAU | 86 | AUUUCGGG CUGAUGAGGCCGUUAGGCCCGAA AUUUCACU | 9505 | |
| 684 | CCCGAAAUU AUACACAU | 87 | AUGUGUAU CUGAUGAGGCCGUUAGGCCCGAA AUUUCGGG | 9506 | |
| 685 | CCGAAAUUA UACACAUG | 88 | CAUGUGUA CUGAUGAGGCCGUUAGGCCCGAA AAUUUCGG | 9507 | |
| 687 | GAAAUUAUA CACAUGAC | 89 | GUCAUGUG CUGAUGAGGCCGUUAGGCCCGAA AUAAUUUC | 9508 | |
| 711 | AGGGAGCUC GUCAUUCC | 90 | GGAAUGAC CUGAUGAGGCCGUUAGGCCCGAA AGCUCCCU | 9509 | |
| 714 | GAGCUCGUC AUUCCCUG | 91 | CAGGGAAU CUGAUGAGGCCGUUAGGCCCGAA ACGAGCUC | 9510 | |
| 717 | CUCGUCAUU CCCUGCCG | 92 | CGGCAGGG CUGAUGAGGCCGUUAGGCCCGAA AUGACGAG | 9511 | |
| 718 | UCGUCAUUC CCUGCCGG | 93 | CCGGCAGG CUGAUGAGGCCGUUAGGCCCGAA AAUGACGA | 9512 | |
| 729 | UGCCGGGUU ACGUCACC | 94 | GGUGACGU CUGAUGAGGCCGUUAGGCCCGAA ACCCGGCA | 9513 | |
| 730 | GCCGGGUUA CGUCACCU | 95 | ACGUGACG CUGAUGAGGCCGUUACGCCCGAA AACCCGCC | 9514 | |
| 734 | GGUUACGUC ACCUAACA | 96 | UGUUAGGU CUGAUGAGGCCGUUAGGCCCGAA ACGUAACC | 9515 | |
| 739 | CGUCACCUA ACAUCACU | 97 | AGUGAUGU CUGAUGAGGCCGUUAGGCCCGAA AGGUCACG | 9516 | |
| 744 | CCUAACAUC ACUGUUAC | 98 | GUAACAGU CUGAUGAGGCCGUUAGGCCCGAA AUGUUAGG | 9517 | |
| 750 | AUCACUGUU ACUUUAAA | 99 | UUUAAAGU CUGAUGAGGCCGUUAGGCCCGAA ACAGUGAU | 9518 | |
| 751 | UCACUGUUA CUUUAAAA | 100 | UUUUAAAG CUGAUGAGGCCGUUAGGCCCGAA AACAGUGA | 9519 | |
| 754 | CUCUUACUU UAAAAAAG | 101 | CUUUUUUA CUGAUGAGGCCGUUAGGCCCGAA AGUAACAG | 9520 | |
| 755 | UGUUACUUU AAAAAAGU | 102 | ACUUUUUU CUGAUGAGGCCGUUAGGCCCGAA AAGUAACA | 9521 | |
| 756 | GUUACUUUA AAAAAGUU | 103 | AACUUUUU CUGAUGAGGCCGUUAGGCCCGAA AAAGUAAC | 9522 | |
| 764 | AAAAAAGUU UCCACUUG | 104 | CAAGUGGA CUGAUGAGGCCGUUAGGCCCGAA ACUUUUUU | 9523 | |
| 765 | AAAAAGUUU CCACUUGA | 105 | UCAAGUGG CUGAUGAGGCCGUUAGGCCCGAA AACUUUUU | 9524 | |
| 766 | AAAAGUUUC CACUUGAC | 106 | GUCAAGUG CUGAUGAGGCCGUUAGGCCCGAA AAACUUUU | 9525 | |
| 771 | UUUCCACUU GACACUUU | 107 | AAAGUGUC CUGAUGAGGCCGUUAGGCCCGAA AGUGGAAA | 9526 | |
| 778 | UUGACACUU UGAUCCCU | 108 | AGGGAUCA CUGAUGAGGCCGUUAGGCCCGAA AGUGUCAA | 9527 | |
| 779 | UGACACUUU GAUCCCUG | 109 | CAGGGAUC CUGAUGAGGCCGUUAGGCCCGAA AAGUGUCA | 9528 | |
| 783 | ACUUUGAUC CCUGAUGG | 110 | CCAUCAGG CUGAUGAGGCCGUUAGGCCCGAA AUCAAAGU | 9529 | |
| 801 | AAACGCAUA AUCUGGGA | 111 | UCCCAGAU CUGAUGAGGCCGUUACGCCCGAA AAGCGUUU | 9530 | |
| 804 | CGCAUAAUC UGGGACAG | 112 | CUGUCCCA CUGAUGAGGCCGUUAGGCCCGAA AUUAUGCG | 9531 | |
| 814 | GGGACAGUA GAAAGGGC | 113 | GCCCUUUC CUGAUGAGGCCGUUAGGCCCGAA ACUGUCCC | 9532 | |
| 824 | AAAGGCCUU CAUCAUAU | 114 | AUAUGAUG CUGAUGAGGCCGUUAGGCCCGAA AGCCCUUU | 9533 | |
| 825 | AAGGGCUUC AUCAUAUC | 115 | GAUAUGAU CUGAUGAGGCCGUUAGGCCCGAA AAGCCCUU | 9534 | |
| 828 | GGCUUCAUC AUAUCAAA | 116 | UUUGAUAU CUGAUGAGGCCCUUAGGCCCGAA AUGAAGCC | 9535 | |
| 831 | UUCAUCAUA UCAAAUGC | 117 | GCAUUUGA CUGAUGAGGCCGUUAGGCCCGAA AUGAUGAA | 9536 | |
| 833 | CAUCAUAUC AAAUGCAA | 118 | UUGCAUUU CUGAUGAGGCCGUUAGGCCCGAA AUAUGAUG | 9537 | |
| 845 | UGCAACGUA CAAAGAAA | 119 | UUUCUUUG CUGAUGAGGCCCUUAGGCCCGAA ACGUUGCA | 9538 | |
| 855 | AAAGAAAUA GGGCUUCU | 120 | AGAAGCCC CUGAUGACGCCGUUAGGCCCGAA AUUUCUUU | 9539 | |
| 861 | AUAGGGCUU CUGACCUG | 121 | CAGGUCAG CUGAUGAGGCCGUUAGGCCCGAA AGCCCUAU | 9540 | |
| 862 | UAGGGCUUC UGACCUGU | 122 | ACAGGUCA CUGAUGAGGCCGUUAGGCCCGAA AAGCCCUA | 9541 | |
| 882 | GCAACAGUC AAUGGGCA | 123 | UGCCCAUU CUGAUGAGGCCGUUAGGCCCGAA ACUGUUGC | 9542 | |
| 892 | AUGGGCAUU UGUAUAAG | 124 | CUUAUACA CUGAUGAGGCCGUUAGGCCCGAA AUGCCCAU | 9543 | |
| 893 | UGGGCAUUU GUAUAAGA | 125 | UCUUAUAC CUGAUGAGGCCGUUAGGCCCGAA AAUGCCCA 9544 | | |
| 896 | GCAUUUGUA UAAGACAA | 126 | UUGUCUUA CUGAUGAGGCCGUUAGGCCCGAA ACAAAUGC | 9545 | |
| 898 | AUUUGUAUA AGACAAAC | 127 | GUUUGUCU CUGAUGAGGCCGUUAGGCCCGAA AUACAAAU | 9546 | |
| 908 | GACAAACUA UCUCACAC | 128 | GUGUGAGA CUGAUGAGGCCGUUAGGCCCGAA AGUUUGUC | 9547 | |
| 910 | CAAACUAUC UCACACAU | 129 | AUGUGUGA CUGAUGAGGCCGUUAGGCCCGAA AUAGUUUG | 9548 | |
| 912 | AACUAUCUC ACACAUCG | 130 | CGAUGUGU CUGAUGAGGCCGUUAGGCCCGAA AGAUAGUU | 9549 | |
| 919 | UCACACAUC GACAAACC | 131 | GGUUUGUC CUGAUGAGGCCGUUAGGCCCGAA AUGUGUGA | 9550 | |
| 931 | AAACCAAUA CAAUCAUA | 132 | UAUGAUUG CUGAUGAGGCCGUUAGGCCCGAA AUUGGUUU | 9551 | |
| 936 | AAUACAAUC AUAGAUGU | 133 | ACAUCUAU CUGAUGAGGCCGUUAGGCCCGAA AUUGUAUU | 9552 | |
| 939 | ACAAUCAUA GAUGUCCA | 134 | UGGACAUC CUGAUGAGGCCGUUAGGCCCGAA AUGAUUGU | 9553 | |
| 945 | AUAGAUGUC CAAAUAAG | 135 | CUUAUUUG CUGAUGAGGCCGUUAGGCCCGAA ACAUCUAU | 9554 | |
| 951 | GUCCAAAUA AGCACACC | 136 | GGUGUGCU CUGAUGAGGCCGUUAGGCCCGAA AUUUGGAC | 9555 | |
| 969 | CGCCCAGUC AAAUUACU | 137 | AGUAAUUU CUGAUGAGGCCGUUAGGCCCGAA ACUGGGCG | 9556 | |
| 974 | AGUCAAAUU ACUUAGAG | 138 | CUCUAAGU CUGAUGAGGCCGUUAGGCCCGAA AUUUGACU | 9557 | |
| 975 | GUCAAAUUA CUUAGAGG | 139 | CCUCUAAG CUGAUGAGGCCGUUAGGCCCGAA AAUUUGAC | 9558 | |
| 978 | AAAUUACUU AGAGGCCA | 140 | UGGCCUCU CUGAUGAGGCCGUUAGGCCCGAA AGUAAUUU | 9559 | |
| 979 | AAUUACUUA GAGGCCAU | 141 | AUGGCCUC CUGAUGAGGCCGUUAGGCCCGAA AAGUAAUU | 9560 | |
| 988 | GAGGCCAUA CUCUUGUC | 142 | GACAAGAG CUGAUGAGGCCGUUAGGCCCGAA AUGGCCUC | 9561 | |
| 991 | GCCAUACUC UUGUCCUC | 143 | GAGGACAA CUGAUGAGGCCGUUAGGCCCGAA AGUAUGGC | 9562 | |
| 993 | CAUACUCU | GUCCUCAA | 144 | UUGAGGAC CUGAUGAGGCCGUUAGGCCCGAA AGAGUAUG | 9563 |
| 996 | ACUCUUGUC CUCAAUUG | 145 | CAAUUGAG CUGAUGAGGCCGUUAGGCCCGAA ACAAGAGU | 9564 | |
| 999 | CUUGUCCUC AAUUGUAC | 146 | GUACAAUU CUGAUGAGGCCGUUAGGCCCGAA AGCACAAG | 9565 | |
| 1003 | UCCUCAAUU GUACUCCU | 147 | ACCAGUAC CUGAUGAGGCCGUUAGGCCCGAA AUUGAGGA | 9566 | |
| 1006 | UCAAUUGUA CUGCUACC | 148 | GGUAGCAG CUGAUGAGGCCGUUAGGCCCGAA ACAAUUGA | 9567 | |
| 1012 | GUACUGCUA CCACUCCC | 149 | GGGAGUGG CUGAUGAGGCCGUUAGGCCCGAA AGCAGUAC | 9568 | |
| 1018 | CUACCACUC CCUUGAAC | 150 | GUUCAAGG CUGAUGAGGCCGUUAGGCCCGAA AGUGGUAG | 9569 | |
| 1022 | CACUCCCUU GAACACGA | 151 | UCGUGUUC CUGAUGAGGCCGUUAGGCCCGAA AGGUAGUG | 9570 | |
| 1035 | ACGAGAGUU CAAAUGAC | 152 | GUCAUUUG CUGAUGAGGCCGUUAGGCCCGAA ACUCUCGU | 9571 | |
| 1036 | CGAGAGUUC AAAUGACC | 153 | GGUCAUUU CUGAUGAGGCCGUUAGGCCCGAA AACUCUCG | 9572 | |
| 1051 | CCUGGAGUU ACCCUGAU | 154 | AUCAGGGU CUGAUGAGGCCGUUAGGCCCGAA ACUCCAGO | 9573 | |
| 1052 | CUGGAGUUA CCCUGAUG | 155 | CAUCAGUG CUGAUGAGGCCGUUAGGCCCGAA AACUCCAG | 9574 | |
| 1069 | AAAAAAAUA AGAGAGCU | 156 | AUCUCUCU CUGAUGAGGCCGUUAGGCCCGAA AUUUUUUU | 9575 | |
| 1078 | AGAGAGCUU CCGUAAGG | 157 | CCUUACGG CUGAUGAGGCCGUUAGGCCCGAA AGCUCUCU | 9576 | |
| 1079 | GAGAGCUUC CGUAAGGC | 158 | GCCUUACG CUGAUGAGGCCGUUAGGCCCGAA AAGCUCUC | 9577 | |
| 1083 | GCUUCCGUA AGGCGACG | 159 | CGUCGCCU CUGAUGAGGCCGUUAGGCCCGAA ACGGAAGC | 9578 | |
| 1095 | CGACGAAUU GACCAAAG | 160 | CUUUGGUC CUGAUGAGGCCGUUAGGCCCGAA AUUCGUCG | 9579 | |
| 1108 | AAAGCAAUU CCCAUGCC | 161 | GGCAUGGG CUGAUGAGGCCGUUAGGCCCGAA AUUGCUUU | 9580 | |
| 1109 | AAGCAAUUC CCAUGCCA | 162 | UGGCAUGG CUGAUGAGGCCGUUAGGCCCGAA AAUUGCUU | 9581 | |
| 1122 | GCCAACAUA UUCUACAG | 163 | CUGUAGAA CUGAUGAGGCCGUUAGGCCCGAA AUGUUGGC | 9582 | |
| 1124 | CAACAUAUU CUACAGUG | 164 | CACUGUAG CUGAUGAGGCCGUUAGGCCCGAA AUAUGUUG | 9583 | |
| 1125 | AACAUAUUC UACAGUGU | 165 | ACACUGUA CUGAUGAGGCCGUUAGGCCCGAA AAUAUGUU | 9584 | |
| 1127 | CAUAUUCUA CAGUGUUC | 166 | GAACACUG CUGAUGAGGCCGUUAGGCCCGAA AGAAUAUG | 9585 | |
| 1134 | UACAGUGUU CUUACUAU | 167 | AUAGUAAG CUGAUGAGGCCGUUAGGCCCGAA ACACUGUA | 9586 | |
| 1135 | ACAGUGUUC UUACUAUU | 168 | AAUAGUAA CUGAUGAGGCCGUUAGGCCCGAA AACACUGU | 9587 | |
| 1137 | AGUGUUCUU ACUAUUGA | 169 | UCAAUAGU CUGAUGAGGCCGUUAGGCCCGAA AGAACACU | 9588 | |
| 1138 | GUGUUCUUA CUAUUGAC | 170 | GUCAAUAG CUGAUGAGGCCGUUAGGCCCGAA AAGAACAC | 9589 | |
| 1141 | UUCUUACUA UUGACAAA | 171 | UUUGUCAA CUGAUGAGGCCGUUAGGCCCGAA AGUAAGAA | 9590 | |
| 1143 | CUUACUAUU GACAAAAU | 172 | AUUUUGUC CUGAUGAGGCCGUUAGGCCCGAA AUAGUAAG | 9591 | |
| 1173 | AAAGGACUU UAUACUUG | 173 | CAAGUAUA CUGAUGAGGCCGUUAGGCCCGAA AGUCCUUU | 9592 | |
| 1174 | AAGGACUUU AUACUUGU | 174 | ACAAGUAU CUGAUGAGGCCGUUAGGCCCGAA AAGUCCUU | 9593 | |
| 1175 | AGGACUUUA UACUUGUC | 175 | GACAAGUA CUGAUGAGGCCGUUAGGCCCGAA AAAGUCCU | 9594 | |
| 1177 | GACUUUAUA CUUGUCGU | 176 | ACGACAAG CUGAUGAGGCCGUUAGGCCCGAA AUAAAGUC | 9595 | |
| 1180 | UUUAUACUU GUCGUGUA | 177 | UACACGAC CUGAUGAGGCCGUUAGGCCCGAA AGUAUAAA | 9596 | |
| 1183 | AUACUUGUC GUGUAAGG | 178 | CCUUACAC CUGAUGAGGCCGUUAGGCCCGAA ACAAGUAU | 9597 | |
| 1188 | UGUCGUGUA AGGAGUGG | 179 | CCACUCCU CUGAUGAGGCCGUUAGGCCCGAA ACACGACA | 9598 | |
| 1202 | UGGACCAUC AUUCAAAU | 180 | AUUUGAAU CUGAUGAGGCCGUUAGGCCCGAA AUGGUCCA | 9599 | |
| 1205 | ACCAUCAUU CAAAUCUG | 181 | CAGAUUUG CUGAUGAGGCCGUUAGGCCCGAA AUGAUGGU | 9600 | |
| 1206 | CCAUCAUUC AAAUCUGU | 182 | ACAGAUUU CUGAUGAGGCCGUUAGGCCCGAA AAUGAUGG | 9601 | |
| 1211 | AUUCAAAUC UGUUAACA | 183 | UGUUAACA CUGAUGAGGCCGUUAGGCCCGAA AUUUGAAU | 9602 | |
| 1215 | AAAUCUGUU AACACCUC | 184 | GAGGUGUU CUGAUGAGGCCGUUAGGCCCGAA ACAGAUUU | 9603 | |
| 1216 | AAUCUGUUA ACACCUCA | 185 | UGAGGUGU CUGAUGAGGCCGUUAGGCCCGAA AACAGAUU | 9604 | |
| 1223 | UAACACCUC AGUGCAUA | 186 | UAUGCACU CUGAUGAGGCCGUUAGGCCCGAA AGGUGUUA | 9605 | |
| 1231 | CAGUGCAUA UAUAUGAU | 187 | AUCAUAUA CUGAUGAGGCCGUUAGGCCCGAA AUGCACUG | 9606 | |
| 1233 | GUGCAUAUA UAUGAUAA | 188 | UUAUCAUA CUGAUGAGGCCGUUAGGCCCGAA AUAUGCAC | 9607 | |
| 1235 | GCAUAUAUA UGAUAAAG | 189 | CUUUAUCA CUGAUGAGGCCGUUAGGCCCGAA AUAUAUGC | 9608 | |
| 1240 | UAUAUGAUA AAGCAUUC | 190 | GAAUGCUU CUGAUGAGGCCGUUAGGCCCGAA AUCAUAUA | 9609 | |
| 1247 | UAAAGCAUU CAUCACUG | 191 | CAGUGAUG CUGAUGAGGCCGUUAGGCCCGAA AUGCUUUA | 9610 | |
| 1248 | AAAGCAUUC AUCACUGU | 192 | ACAGUGAU CUGAUGAGGCCGUUAGGCCCGAA AAUGCUUU | 9611 | |
| 1251 | GCAUUCAUC ACUGUGAA | 193 | UUCACAGU CUGAUGAGGCCGUUAGGCCCGAA AUGAAUGC | 9612 | |
| 1264 | UGAAACAUC GAAAACAG | 194 | CUGUUUUC CUGAUGAGGCCGUUAGGCCCGAA AUGUUUCA | 9613 | |
| 1281 | CAGGUGCUU GAAACCGU | 195 | ACGGUUUC CUGAUGAGGCCGUUAGGCCCGAA AGCACCUG | 9614 | |
| 1290 | GAAACCGUA GCUGGCAA | 196 | UUGCCAGC CUGAUGAGGCCGUUAGGCCCGAA ACGCUUUC | 9615 | |
| 1304 | CAAGCGGUC UUACCGGC | 197 | GCCGGUAA CUGAUGAGGCCGUUAGGCCCGAA ACCGCUUC | 9616 | |
| 1306 | AGCGGUCUU ACCCGCUC | 198 | GAGCCGGU CUGAUGAGGCCGUUAGGCCCGAA AGACCGCU | 9617 | |
| 1307 | GCGGUCUUA CCGGCUCU | 199 | AGAGCCGG CUGAUGAGGCCGUUAGGCCCGAA AAGACCGC | 9618 | |
| 1314 | UACCGGCUC UCUAUGAA | 200 | UUCAUAGA CUGAUGAGGCCGUUAGGCCCGAA AGCCCGUA | 9619 | |
| 1316 | CCGGCUCUC UAUGAAAG | 201 | CUUUCAUA CUGAUGAGGCCGUUAGGCCCGAA AGAGCCGG | 9620 | |
| 1318 | GGCUCUCUA UGAAAGUG | 202 | CACUUUCA CUGAUGAGGCCGUUAGGCCCGAA AGAGAGCC | 9621 | |
| 1334 | GAAGGCAUU UCCCUCGC | 203 | GCGAGGGA CUCAUGAGGCCGUUAGGCCCGAA AUGCCUUC | 9622 | |
| 1335 | AAGGCAUUU CCCUCGCC | 204 | GGCGAGGG CUGAUGAGGCCGUUAGGCCCGAA AAUGCCUU | 9623 | |
| 1336 | AGGCAUUUC CCUCGCCG | 205 | CCGCGAGG CUGAUGAGGCCGUUAGGCCCGAA AAAUGCCU | 9624 | |
| 1340 | AUUUCCCUC GCCGGAAG | 206 | CUUCCGGC CUGAUGAGGCCGUUAGGCCCGAA AGGGAAAU | 9625 | |
| 1350 | CCGGAAGUU GUAUGGUU | 207 | AACCAUAC CUGAUGAGGCCGUUAGGCCCGAA ACUUCCGG | 9626 | |
| 1353 | GAAGUUGUA UGCUUAAA | 208 | UUUAACCA CUGAUGAGGCCGUUAGGCCCGAA ACAACUUC | 9627 | |
| 1358 | UGUAUGGUU AAAAGAUG | 209 | CAUCUUUU CUGAUGAGGCCGUUAGGCCCGAA ACCAUACA | 9628 | |
| 1359 | GUAUGGUUA AAAGAUGG | 210 | CCAUCUUU CUGAUGACGCCGUUAGGCCCGAA AACCAUAC | 9629 | |
| 1370 | AGAUGGGUU ACCUGCGA | 211 | UCGCAGGU CUGAUGACGCCGUUACGCCCGAA ACCCAUCU | 9630 | |
| 1371 | GAUGGGUUA CCUGCGAC | 212 | GUCGCAGG CUGAUGAGGCCGUUAGGCCCGAA AACCCAUC | 9631 | |
| 1388 | UGAGAAAUC UCCUCGCU | 213 | AGCGAGCA CUCAUGAGGCCGUUAGGCCCGAA AUUUCUCA | 9632 | |
| 1393 | AAUCUGCUC GCUAUUUG | 214 | CAAAUAGC CUCAUGAGGCCGUUAGGCCCGAA AGCAGAUU | 9633 | |
| 1397 | UGCUCGCUA UUUGACUC | 215 | GAGUCAAA CUGAUGAGCCCGUUAGGCCCGAA AGCGAGCA | 9634 | |
| 1399 | CUCGCUAUU UGACUCCU | 216 | ACCAGUCA CUGAUGAGGCCGUUAGGCCCGAA AUAGCGAG | 9635 | |
| 1400 | UCGCUAUUU GACUCGUG | 217 | CACGAGUC CUGAUGAGGCCGUUAGGCCCGAA AAUAGCGA | 9636 | |
| 1405 | AUUUGACUC GUGGCUAC | 218 | GUAGCCAC CUGAUGAGGCCGUUAGGCCCGAA AGUCAAAU | 9637 | |
| 1412 | UCGUGGCUA CUCGUUAA | 219 | UUAACGAG CUGAUGAGGCCGUUAGGCCCGAA AGCCACGA | 9638 | |
| 1415 | UGUCUACUC GUUAAUUA | 220 | UAAUUAAC CUGAUGAGGCCGUUAGGCCCGAA AGUAGCCA | 9639 | |
| 1418 | CUACUCGUU AAUUAUCA | 221 | UGAUAAUU CUGAUGAGGCCGUUAGGCCCGAA ACCAGUAG | 9640 | |
| 1419 | UACUCGUUA AUUAUCAA | 222 | UUGAUAAU CUGAUGAGGCCGUUAGGCCCGAA AACGAGUA | 9641 | |
| 1422 | UCGUUAAUU AUCAAGGA | 223 | UCCUUGAU CUGAUGAGGCCGUUAGGCCCGAA AUUAACGA | 9642 | |
| 1423 | CGUUAAUUA UCAAGGAC | 224 | GUCCUUGA CUGAUGAGGCCGUUAGGCCCGAA AAUUAACG | 9643 | |
| 1425 | UUAAUUAUC AAGGACGU | 225 | ACGUCCUU CUGAUGAGGCCGUUAGGCCCGAA AUAAUUAA | 9644 | |
| 1434 | AAGGACGUA ACUGAAGA | 226 | UCUUCAGU CUGAUGAGGCCGUUAGGCCCGAA ACGUCCUU | 9645 | |
| 1456 | CAGGGAAUU AUACAAUC | 227 | GAUUGUAU CUGAUGAGGCCGUUAGGCCCGAA AUUCCCUG | 9646 | |
| 1457 | AGGGAAUUA UACAAUCU | 228 | AGAUUGUA CUGAUGAGGCCGUUAGGCCCGAA AAUUCCCU | 9647 | |
| 1459 | GGAAUUAUA CAAUCUUG | 229 | CAAGAUUG CUGAUGAGGCCGUUAGGCCCGAA AUAAUUCC | 9648 | |
| 1464 | UAUACAAUC UUGCUGAG | 230 | CUCAGCAA CUGAUGAGGCCGUUAGGCCCGAA AUUGUAUA | 9649 | |
| 1466 | UACAAUCUU GCUGAGCA | 231 | UGCUCAGC CUGAUGAGGCCGUUAGGCCCGAA AGAUUGUA | 9650 | |
| 1476 | CUGAGCAUA AAACAGUC | 232 | GACUGUUU CUGAUGAGGCCGUUAGGCCCGAA AUGCUCAG | 9651 | |
| 1484 | AAAACAGUC AAAUGUGU | 233 | ACACAUUU CUGAUGAGGCCGUUAGGCCCGAA ACUGUUUU | 9652 | |
| 1493 | AAAUGUGUU UAAAAACC | 234 | GGUUUUUA CUGAUGAGGCCGUUAGGCCCGAA ACACAUUU | 9653 | |
| 1494 | AAUGUGUUU AAAAACCU | 235 | AGGUUUUU CUGAUGAGGCCGUUAGGCCCGAA AACACAUU | 9654 | |
| 1495 | AUGUGUUUA AAAACCUC | 236 | GAGGUUUU CUGAUGAGGCCGUUAGGCCCGAA AAACACAU | 9655 | |
| 1503 | AAAAACCUC ACUGCCAC | 237 | GUGGCAGU CUGAUGAGGCCGUUAGGCCCGAA AGGUUUUU | 9656 | |
| 1513 | CUGCCACUC UAAUUGUC | 238 | GACAAUUA CUGAUGAGGCCGUUAGGCCCGAA AGUGGCAG | 9657 | |
| 1515 | GCCACUCUA AUUGUCAA | 239 | UUGACAAU CUGAUGAGGCCGUUAGGCCCGAA AGAGUGUC | 9658 | |
| 1518 | ACUCUAAUU GUCAAUGU | 240 | ACAUUGAC CUGAUGAGGCCGUUAGGCCCGAA AUUAGAGU | 9659 | |
| 1521 | CUAAUUGUC AAUGUGAA | 241 | UUCACAUU CUGAUGAGGCCGUUAGGCCCGAA ACAAUUAG | 9660 | |
| 1539 | CCCCAGAUU UACGAAAA | 242 | UUUUCGUA CUGAUGAGGCCGUUAGGCCCGAA AUCUGGGG | 9661 | |
| 1540 | CCCAGAUUU ACGAAAAG | 243 | CUUUUCGU CUGAUGAGGCCGUUAGGCCCGAA AAUCUGGG | 9662 | |
| 1541 | CCAGAUUUA CGAAAACG | 244 | CCUUUUCG CUGAUGAGGCCGUUAGGCCCGAA AAAUCUGG | 9663 | |
| 1556 | GCCCGUGUC AUCGUUUC | 245 | GAAACGAU CUGAUGAGGCCGUUAGGCCCGAA ACACGGCC | 9664 | |
| 1559 | CGUGUCAUC GUUUCCAG | 246 | CUGGAAAC CUCAUGAGGCCGUUAGGCCCGAA AUGACACG | 9665 | |
| 1562 | GUCAUCGUU UCCAGACC | 247 | GGUCUGGA CUGAUGAGGCCGUUAGGCCCGAA ACCAUGAC | 9666 | |
| 1563 | UCAUCGUUU CCAGACCC | 248 | GGGUCUGG CUGAUGAGGCCGUUAGGCCCGAA AACGAUGA | 9667 | |
| 1564 | CAUCCUUUC CAGACCCG | 249 | CGGGUCUG CUGAUGAGGCCGUUAGGCCCGAA AAACGAUG | 9668 | |
| 1576 | ACCCGGCUC UCUACCCA | 250 | UGGGUAGA CUGAUGAGGCCGUUAGGCCCGAA AGCCGGGU | 9669 | |
| 1578 | CCGGCUCUC UACCCACU | 251 | AGUCGGUA CUGAUGAGGCCGUUAGGCCCGAA AGAGCCGG | 9670 | |
| 1580 | GGCUCUCUA CCCACUGG | 252 | CCAGUGGG CUGAUGAGGCCGUUAGGCCCGAA AGAGAGCC | 9671 | |
| 1602 | AGACAAAUC CUGACUUG | 253 | CAAGUCAG CUGAUGAGGCCGUUAGGCCCGAA AUUUGUCU | 9672 | |
| 1609 | UCCUGACUU GUACCGCA | 254 | UGCGGUAC CUGAUGAGGCCGUUAGGCCCGAA AGUCAGGA | 9673 | |
| 1612 | UGACUUGUA CCGCAUAU | 255 | AUAUGCGG CUGAUGAGGCCGUUAGGCCCGAA ACAAGUCA | 9674 | |
| 1619 | UACCGCAUA UGGUAUCC | 256 | GGAUACCA CUGAUGAGGCCGUUAGGCCCGAA AUGCGGUA | 9675 | |
| 1624 | CAUAUGGUA UCCCUCAA | 257 | UUGAGGGA CUGAUGAGGCCGUUAGGCCCGAA ACCAUAUG | 9676 | |
| 1626 | UAUGGUAUC CCUCAACC | 258 | GGUUGAGG CUGAUGAGGCCGUUAGGCCCGAA AUACCAUA | 9677 | |
| 1630 | GUAUCCCUC AACCUACA | 259 | UGUAGGUU CUGAUGAGGCCGUUAGGCCCGAA AGGGAUAC | 9678 | |
| 1636 | CUCAACCUA CAAUCAAG | 260 | CUUGAUUG CUGAUGAGGCCGUUAGGCCCGAA AGGUUGAG | 9679 | |
| 1641 | CCUACAAUC AAGUGGUU | 261 | AACCACUU CUGAUGAGGCCGUUAGGCCCGAA AUUGUAGG | 9680 | |
| 1649 | CAAGUGGUU CUGGCACC | 262 | GGUGCCAG CUGAUGAGGCCGUUAGGCCCGAA ACCACUUG | 9681 | |
| 1650 | AAGUGGUUC UGGCACCC | 263 | GGGUGCCA CUGAUGAGGCCGUUAGGCCCGAA AACCACUU | 9682 | |
| 1663 | ACCCCUGUA ACCAUAAU | 264 | AUUAUGGU CUGAUGAGGCCGUUAGGCCCGAA ACAGGGGU | 9683 | |
| 1669 | GUAACCAUA AUCAUUCC | 265 | GGAAUGAU CUGAUGAGGCCGUUAGGCCCGAA AUGGUUAC | 9684 | |
| 1672 | ACCAUAAUC AUUCCGAA | 266 | UUCGGAAU CUGAUGAGGCCGUUAGGCCCGAA AUUAUGGU | 9685 | |
| 1675 | AUAAUCAUU CCGAAGCA | 267 | UGCUUCGG CUGAUGAGGCCGUUAGGCCCGAA AUGAUUAU | 9686 | |
| 1676 | UAAUCAUUC CGAAGCAA | 268 | UUGCUUCG CUGAUGAGGCCGUUAGGCCCGAA AAUGAUUA | 9687 | |
| 1694 | GUGUGACUU UUGUUCCA | 269 | UGGAACAA CUGAUGAGGCCGUUAGGCCCGAA AGUCACAC | 9688 | |
| 1695 | UGUGACUUU UGUUCCAA | 270 | UUGGAACA CUGAUGAGGCCGUUAGGCCCGAA AAGUCACA | 9689 | |
| 1696 | GUGACUUUU GUUCCAAU | 271 | AUUGGAAC CUGAUGAGGCCGUUAGGCCCGAA AAAGUCAC | 9690 | |
| 1699 | ACUUUUGUU CCAAUAAU | 272 | AUUAUUGG CUGAUGAGGCCGUUAGGCCCGAA ACAAAAGU | 9691 | |
| 1700 | CUUUUGUUC CAAUAAUG | 273 | CAUUAUUG CUGAUGAGGCCGUUAGGCCCGAA AACAAAAG | 9692 | |
| 1705 | GUUCCAAUA AUGAAGAG | 274 | CUCUUCAU CUGAUGAGGCCGUUAGGCCCGAA AUUGGAAC | 9693 | |
| 1715 | UGAAGAGUC CUUUAUCC | 275 | GGAUAAAG CUGAUGAGGCCGUUAGGCCCGAA ACUCUUCA | 9694 | |
| 1718 | AGAGUCCUU UAUCCUGG | 276 | CCAGGAUA CUGAUGAGGCCGUUAGGCCCGAA AGGACUCU | 9695 | |
| 1719 | GAGUCCUUU AUCCUGGA | 277 | UCCAGGAU CUGAUGAGGCCGUUAGGCCCGAA AAGGACUC | 9696 | |
| 1720 | AGUCCUUUA UCCUGGAU | 278 | AUCCAGGA CUGAUGAGGCCGUUAGGCCCGAA AAAGGACU | 9697 | |
| 1722 | UCCUUUAUC CUGGAUGC | 279 | GCAUCCAG CUGAUGAGGCCGUUAGGCCCGAA AUAAAGGA | 9698 | |
| 1755 | AACAGAAUU GAGAGCAU | 280 | AUGCUCUC CUGAUGAGGCCGUUAGGCCCGAA AUUCUGUU | 9699 | |
| 1764 | GAGAGCAUC ACUCAGCG | 281 | CGCUGAGU CUGAUGAGGCCGUUAGGCCCGAA AUCCUCUC | 9700 | |
| 1768 | GCAUCACUC AGCGCAUG | 282 | CAUGCGCU CUGAUGAGGCCGUUAGGCCCGAA AGUGAUGC | 9701 | |
| 1782 | AUGGCAAUA AUAGAAGG | 283 | CCUUCUAU CUGAUGAGGCCGUUAGGCCCGAA AUUGCCAU | 9702 | |
| 1785 | GCAAUAAUA GAAGGAAA | 284 | UUUCCUUC CUGAUGAGGCCGUUAGGCCCGAA AUUAUUGC | 9703 | |
| 1798 | GAAAGAAUA AGAUGGCU | 285 | AGCCAUCU CUGAUGAGGCCGUUAGGCCCGAA AUUCUUUC | 9704 | |
| 1807 | AGAUGGCUA GCACCUUG | 286 | CAAGGUGC CUGAUGAGGCCGUUAGGCCCGAA AGCCAUCU | 9705 | |
| 1814 | UAGCACCUU GGUUGUGG | 287 | CCACAACC CUGAUGAGGCCGUUAGGCCCGAA AGGUGCUA | 9706 | |
| 1818 | ACCUUGGUU GUGGCUGA | 288 | UCAGCCAC CUGAUGAGGCCGUUAGGCCCGAA ACCAAGGU | 9707 | |
| 1829 | GOCUGACUC UAGAAUUU | 289 | AAAUUCUA CUGAUGAGGCCGUUAGGCCCGAA AGUCAGCC | 9708 | |
| 1831 | CUGACUCUA GAAUUUCU | 290 | AGAAAUUC CUGAUGAGGCCGUUAGGCCCGAA AGAGUCAG | 9709 | |
| 1836 | UCUAGAAUU UCUGGAAU | 291 | AUUCCAGA CUGAUGAGGCCGUUAGGCCCGAA AUUCUAGA | 9710 | |
| 1837 | CUAGAAUUU CUGGAAUC | 292 | GAUUCCAG CUGAUGAGGCCGUUAGGCCCGAA AAUUCUAG | 9711 | |
| 1838 | UAGAAUUUC UGGAAUCU | 293 | AGAUUCCA CUGAUGAGGCCGUUAGGCCCGAA AAAUUCUA | 9712 | |
| 1845 | UCUGGAAUC UACAUUUG | 294 | CAAAUGUA CUGAUGAGGCCGUUAGGCCCGAA AUUCCAGA | 9713 | |
| 1847 | UGGAAUCUA CAUUUGCA | 295 | UGCAAAUC CUGAUGAGGCCGUUAGGCCCGAA AGAUUCCA | 9714 | |
| 1851 | AUCUACAUU UGCAUAGC | 296 | GCUAUCCA CUGAUGAGGCCGUUAGGCCCGAA AUGUAGAU | 9715 | |
| 1852 | UCUACAUUU GCAUAGCU | 297 | AGCUAUGC CUGAUGAGGCCGUUAGGCCCGAA AAUCUAGA | 9716 | |
| 1857 | AUUUGCAUA GCUUCCAA | 298 | UUGGAAGC CUGAUGAGGCCGUUAGGCCCGAA AUGCAAAU | 9717 | |
| 1861 | GCAUAGCUU CCAAUAAA | 299 | UUUAUUGG CUGAUGAGGCCGUUAGGCCCGAA AGCUAUGC | 9718 | |
| 1862 | CAUAGCUUC CAAUAAAG | 300 | CUUUAUUG CUGAUGAGGCCGUUAGGCCCGAA AAGCUAUC | 9719 | |
| 1867 | CUUCCAAUA AAGUUGGG | 301 | CCCAACUU CUGAUGAGCCCGUUAGGCCCGAA AUUGGAAG | 9720 | |
| 1872 | AAUAAAGUU GGGACUGU | 302 | ACAGUCCC CUGAUGAGGCCGUUAGGCCCGAA ACUUUAUU | 9721 | |
| 1893 | AGAAACAUA AGCUUUUA | 303 | UAAAAGCU CUGAUGAGGCCGUUAGGCCCGAA AUGUUUCU | 9722 | |
| 1898 | CAUAAGCUU UUAUAUCA | 304 | UGAUAUAA CUGAUGAGGCCGUUAGGCCCGAA AGCUUAUG | 9723 | |
| 1899 | AUAAGCUUU UAUAUCAC | 305 | GUGAUAUA CUGAUGAGGCCGUUAGGCCCGAA AAGCUUAU | 9724 | |
| 1900 | UAAGCUUUU AUAUCACA | 306 | UGUGAUAU CUGAUGAGGCCGUUAGGCCCGAA AAAGCUUA | 9725 | |
| 1901 | AAGCUUUUA UAUCACAG | 307 | CUGUGAUA CUGAUGAGGCCGUUAGGCCCGAA AAAAGCUU | 9726 | |
| 1903 | GCUUUUAUA UCACACAU | 308 | AUCUGUGA CUGAUGAGGCCGUUAGGCCCGAA AUAAAAGC | 9727 | |
| 1905 | UUUUAUAUC ACAGAUGU | 309 | ACAUCUGU CUGAUGAGGCCGUUAGGCCCGAA AUAUAAAA | 9728 | |
| 1925 | AAAUGGGUU UCAUGUUA | 310 | UAACAUGA CUGAUGAGGCCGUUAGGCCCGAA ACCCAUUU | 9729 | |
| 1926 | AAUGGGUUU CAUGUUAA | 311 | UUAACAUG CUGAUGAGGCCGUUAGGCCCGAA AACCCAUU | 9730 | |
| 1927 | AUGGGUUUC AUGUUAAC | 312 | GUUAACAU CUGAUGAGGCCGUUAGGCCCGAA AAACCCAU | 9731 | |
| 1932 | UUUCAUGUU AACUUGGA | 313 | UCCAAGUU CUGAUGAGGCCGUUAGGCCCGAA ACAUGAAA | 9732 | |
| 1933 | UUCAUGUUA ACUUGGAA | 314 | UUCCAAGU CUGAUGAGGCCGUUAGGCCCGAA AACAUGAA | 9733 | |
| 1937 | UGUUAACUU GGAAAAAA | 315 | UUUUUUCC CUGAUGAGGCCGUUAGGCCCGAA AGUUAACA | 9734 | |
| 1976 | GAAACUGUC UUGCACAG | 316 | CUGUGCAA CUGAUGAGGCCGUUAGGCCCGAA ACAGUUUC | 9735 | |
| 1978 | AACUGUCUU GCACAGUU | 317 | AACUGUGC CUGAUGAGGCCGUUAGGCCCGAA AGACAGUU | 9736 | |
| 1986 | UGCACAGUU AACAAGUU | 318 | AACUUGUU CUGAUGAGGCCGUUAGGCCCGAA ACUGUOCA | 9737 | |
| 1987 | GCACAGUUA ACAAGUUC | 319 | GAACUUGU CUGAUGAGGCCGUUAGGCCCGAA AACUGUGC | 9738 | |
| 1994 | UAACAAGUU CUUAUACA | 320 | UGUAUAAG CUGAUGAGGCCGUUAGGCCCGAA ACUUGUUA | 9739 | |
| 1995 | AACAAGUUC UUAUACAG | 321 | CUGUAUAA CUGAUGAGGCCGUUAGGCCCGAA AACUUGUU | 9740 | |
| 1997 | CAAGUUCUU AUACAGAG | 322 | CUCUGUAU CUGAUGAGGCCGUUAGGCCCGAA AGAACUUG | 9741 | |
| 1998 | AAGUUCUUA UACAGAGA | 323 | UCUCUGUA CUGAUGAGGCCGUUAGGCCCGAA AAGAACUU | 9742 | |
| 2000 | GUUCUUAUA CAGAGACG | 324 | CGUCUCUG CUGAUGAGGCCGUUAGGCCCGAA AUAAGAAC | 9743 | |
| 2010 | AGAGACGUU ACUUGGAU | 325 | AUCCAAGU CUGAUGAGGCCGUUAGGCCCGAA ACGUCUCU | 9744 | |
| 2011 | GAGACGUUA CUUGGAUU | 326 | AAUCCAAG CUGAUGAGGCCGUUAGGCCCGAA AACGUCUC | 9745 | |
| 2014 | ACGUUACUU GGAUUUUA | 327 | UAAAAUCC CUGAUGAGGCCGUUAGGCCCGAA AGUAACGU | 9746 | |
| 2019 | ACUUGGAUU UUACUGCG | 328 | CGCAGUAA CUGAUGAGGCCGUUAGGCCCGAA AUCCAAGU | 9747 | |
| 2020 | CUUGGAUUU UACUGCGG | 329 | CCGCAGUA CUGAUGAGGCCGUUAGGCCCGAA AAUCCAAG | 9748 | |
| 2021 | UUGGAUUUU ACUGCGGA | 330 | UCCGCAGU CUGAUGAGGCCGUUAGGCCCGAA AAAUCCAA | 9749 | |
| 2022 | UGGAUUUUA CUGCGGAC | 331 | GUCCGCAG CUGAUGAGGCCGUUAGGCCCGAA AAAAUCCA | 9750 | |
| 2034 | CGGACAGUU AAUAACAG | 332 | CUGUUAUU CUGAUGAGGCCGUUAGGCCCGAA ACUGUCCG | 9751 | |
| 2035 | GGACAGUUA AUAACAGA | 333 | UCUGUUAU CUGAUGAGGCCGUUAGGCCCGAA AACUGUCC | 9752 | |
| 2038 | CAGUUAAUA ACAGAACA | 334 | UGUUCUGU CUGAUGAGGCCGUUAGGCCCGAA AUUAACUG | 9753 | |
| 2054 | AAUGCACUA CAGUAUUA | 335 | UAAUACUG CUGAUGAGGCCGUUAGGCCCGAA AGUGCAUU | 9754 | |
| 2059 | ACUACAGUA UUAGCAAG | 336 | CUUGCUAA CUGAUGAGGCCGUUAGGCCCGAA ACUGUAGU | 9755 | |
| 2061 | UACAGUAUU AGCAAGCA | 337 | UGCUUGCU CUGAUGAGGCCGUUAGGCCCGAA AUACUGUA | 9756 | |
| 2062 | ACAGUAUUA GCAAGCAA | 338 | UUGCUUGC CUGAUGAGGCCGUUAGGCCCGAA AAUACUGU | 9757 | |
| 2082 | AUGGCCAUC ACUAAGGA | 339 | UCCUUAGU CUGAUGAGGCCGUUAGGCCCGAA AUGGCCAU | 9758 | |
| 2086 | CCAUCACUA AGGAGCAC | 340 | GUGCUCCU CUGAUGAGGCCGUUAGGCCCGAA AGUGAUGG | 9759 | |
| 2096 | GGAGCACUC CAUCACUC | 341 | GAGUGAUG CUGAUGAGGCCGUUAGGCCCGAA AGUGCUCC | 9760 | |
| 2100 | CACUCCAUC ACUCUUAA | 342 | UUAAGAGU CUGAUGAGGCCGUUAGGCCCGAA AUGGAGUG | 9761 | |
| 2104 | CCAUCACUC UUAAUCUU | 343 | AAGAUUAA CUGAUGAGGCCGUUAGGCCCGAA AGUGAUGG | 9762 | |
| 2106 | AUCACUCUU AAUCUUAC | 344 | GUAAGAUU CUGAUGAGGCCGUUAGGCCCGAA ACAGUGAU | 9763 | |
| 2107 | UCACUCUUA AUCUUACC | 345 | GGUAAGAU CUGAUGAGGCCGUUAGGCCCGAA AAGAGUGA | 9764 | |
| 2110 | CUCUUAAUC UUACCAUC | 346 | GAUGGUAA CUGAUGAGGCCGUUAGGCCCGAA AUUAAGAG | 9765 | |
| 2112 | CUUAAUCUU ACCAUCAU | 347 | AUGAUGGU CUGAUGAGGCCGUUAGCCCCGAA AGAUUAAG | 9766 | |
| 2113 | UUAAUCUUA CCAUCAUG | 348 | CAUGAUGG CUGAUGAGGCCGUUAGCCCCGAA AAGAUUAA | 9767 | |
| 2118 | CUUACCAUC AUGAAUGU | 349 | ACAUUCAU CUGAUGAGGCCGUUAGGCCCGAA AUGGUAAG | 9768 | |
| 2127 | AUGAAUGUU UCCCUGCA | 350 | UCCAUGGA CUGAUGAGGCCGUUAGCCCCGAA ACAUUCAU | 9769 | |
| 2128 | UGAAUGUUU CCCUGCAA | 351 | CUCCAGOG CUGAUGAGGCCGUUAGGCCCGAA AACAUUCA | 9770 | |
| 2129 | GAAUGUUUC CCUGCAAG | 352 | CUUGCAGG CUGAUGAGGCCGUUAGGCCCGAA AAACAUUC | 9771 | |
| 2140 | UGCAAGAUU CAGGCACC | 353 | GGUGCCUG CUGAUGAGGCCGUUAGGCCCGAA AUCUUGCA | 9772 | |
| 2141 | GCAAGAUUC AGGCACCU | 354 | AGGUGCCU CUGAUGAGGCCGUUAGGCCCGAA AAUCUUGC | 9773 | |
| 2150 | AGGCACCUA UGCCUGCA | 355 | UGCAGGCA CUGAUGAGGCCGUUAGGCCCGAA AGGUGCCU | 9774 | |
| 2172 | AGGAAUGUA UACACAGG | 356 | CCUGUGUA CUGAUGAGGCCGUUAGGCCCGAA ACAUUCCU | 9775 | |
| 2174 | GAAUGUAUA CACAGGGG | 357 | CCCCUGUG CUGAUGAGGCCGUUAGGCCCGAA AUACAUUC | 9776 | |
| 2190 | GAAGAAAUC CUCCAGAA | 358 | UCCUGGAG CUGAUGAGGCCGUUAGGCCCGAA AUCUCUUC | 9777 | |
| 2193 | GAAAUCCUC CAGAAGAA | 359 | UUCUUCUG CUGAUGAGGCCGUUAGGCCCGAA AGGAUUUC | 9778 | |
| 2208 | AAAGAAAUU ACAAUCAG | 360 | CUGAUUGU CUGAUGAGGCCGUUAGGCCCGAA AUUUCUUU | 9779 | |
| 2209 | AAGAAAUUA CAAUCAGA | 361 | UCUGAUUG CUGAUGAGGCCGUUAGGCCCGAA AAUUUCUU | 9780 | |
| 2214 | AUUACAAUC AGAGAUCA | 362 | UGAUCUCU CUGAUGAGGCCGUUAGGCCCGAA AUUGUAAU | 9781 | |
| 2221 | UCAGAGAUC AGGAAGCA | 363 | UGCUUCCU CUGAUGAGGCCGUUAGGCCCGAA AUCUCUGA | 9782 | |
| 2234 | AGCACCAUA CCUCCUGC | 364 | GCAGGAGG CUGAUGAGGCCGUUAGGCCCGAA AUGGUGCU | 9783 | |
| 2238 | CCAUACCUC CUGCGAAA | 365 | UUUCGCAG CUGAUGAGGCCGUUAGGCCCGAA AGGUAUGG | 9784 | |
| 2250 | CGAAACCUC AGUGAUCA | 366 | UGAUCACU CUGAUGAGGCCGUUAGGCCCGAA AGGUUUCG | 9785 | |
| 2257 | UCAGUGAUC ACACAGUG | 367 | CACUGUGU CUGAUGAGGCCGUUAGGCCCGAA AUCACUGA | 9786 | |
| 2271 | GUGGCCAUC AGCAGUUC | 368 | GAACUGCU CUGAUGAGGCCGUUAGGCCCGAA AUGGCCAC | 9787 | |
| 2278 | UCAGCAGUU CCACCACU | 369 | AGUGGUGG CUGAUGAGGCCGUUAGGCCCGAA ACUCCUGA | 9788 | |
| 2279 | CAGCAGUUC CACCACUC | 370 | AAGUGGUG CUGAUGAGGCCGUUAGGCCCGAA AACUGCUG | 9789 | |
| 2287 | CCACCACUU UAGACUGU | 371 | ACAGUCUA CUGAUGAGGCCGUUAGGCCCGAA AGUGGUGG | 9790 | |
| 2288 | CACCACUUU AGACUGUC | 372 | GACAGUCU CUGAUGAGGCCGUUAGGCCCGAA AAGUGGUG | 9791 | |
| 2289 | ACCACUUUA GACUGUCA | 373 | UGACAGUC CUGAUGAGGCCGUUAGGCCCGAA AAAGUGGU | 9792 | |
| 2296 | UAGACUGUC AUGCUAAU | 374 | AUUAGCAU CUGAUGAGGCCGUUAGGCCCGAA ACAGUCUA | 9793 | |
| 2302 | GUCAUGCUA AUGGUGUC | 375 | GACACCAU CUGAUGAGGCCGUUAGGCCCGAA AGCAUGAC | 9794 | |
| 2310 | AAUGGUGUC CCCGAGCC | 376 | GGCUCGGG CUGAUGAGGCCGUUAGGCCCGAA ACACCAUU | 9795 | |
| 2320 | CCGAGCCUC AGAUCACU | 377 | AGUGAUCU CUGAUGAGGCCGUUAGGCCCGAA AGGCUCGG | 9796 | |
| 2325 | COUCAGAUC ACUUGGUU | 378 | AACCAAGU CUGAUGAGGCCGUUAGGCCCGAA AUCUGAGG | 9797 | |
| 2329 | AGAUCACUU GGUUUAAA | 379 | UUUAAACC CUGAUGAGGCCGUUAGGCCCGAA AGUGAUCU | 9798 | |
| 2333 | CACUUGGUU UAAAAACA | 380 | UGUUUUUA CUGAUGAGGCCGUUAGGCCCGAA ACCAAGUG | 9799 | |
| 2334 | ACUUGGUUU AAAAACAA | 381 | UUGUUUUU CUGAUGAGGCCGUUAGGCCCGAA AACCAAGU | 9800 | |
| 2335 | CUUGGUUUA AAAACAAC | 382 | GUUGUUUU CUGAUGAGGCCGUUAGGCCCGAA AAACCAAG | 9801 | |
| 2352 | CACAAAAUA CAACAAGA | 383 | UCUUGUUG CUGAUGAGGCCGUUAGGCCCGAA AUUUUGUG | 9802 | |
| 2370 | CCUGGAAUU AUUUUAGG | 384 | CCUAAAAU CUGAUGAGGCCGUUAGGCCCGAA AUUCCAGG | 9803 | |
| 2371 | CUGGAAUUA UUUUAGGA | 385 | UCCUAAAA CUGAUGAGGCCGUUAGGCCCGAA AAUUCCAG | 9804 | |
| 2373 | GGAAUUAUU UUAGGACC | 386 | GGUCCUAA CUGAUGAGGCCGUUAGGCCCGAA AUAAUUCC | 9805 | |
| 2374 | GAAUUAUUU UAGGACCA | 387 | UGGUCCUA CUGAUGAGGCCGUUAGGCCCGAA AAUAAUUC | 9806 | |
| 2375 | AAUUAUUUU AGGACCAG | 388 | CUGGUCCU CUGAUGAGGCCGUUAGGCCCGAA AAAUAAUU | 9807 | |
| 2376 | AUUAUUUUA GGACCAGG | 389 | CCUGGUCC CUGAUGAGGCCGUUAGGCCCGAA AAAAUAAU | 9808 | |
| 2399 | CACOCUGUC UAUUGAAA | 390 | UUUCAAUA CUGAUGAGGCCGUUAGGCCCGAA ACAGCGUG | 9809 | |
| 2400 | ACGCUGUUU AUUGAAAG | 391 | CUUUCAAU CUGAUGAGGCCGUUAGGCCCGAA AACAGCGU | 9810 | |
| 2401 | CGCUGUUUA UUGAAAGA | 392 | UCUUUCAA CUGAUGAGGCCGUUAGGCCCGAA AAACAGCG | 9811 | |
| 2403 | CUGUUUAUU GAAAGAGU | 393 | ACUCUUUC CUGAUGAGGCCGUUAGGCCCGAA AUAAACAG | 9812 | |
| 2412 | GAAAGAGUC ACAGAAGA | 394 | UCUUCUGU CUCGAUGAGGCCGUUAGCCCGAA ACUCUUUC | 9813 | |
| 2433 | GAAGGUGUC UAUCACUG | 395 | CAGUGAUA CUGAUGAGGCCGUUAGGCCCGAA ACACCUUC | 9814 | |
| 2435 | AGGUGUCUA UCACUCCA | 396 | UGCAGUGA CUGAUGAGGCCGUUAGGCCCGAA AGACACCU | 9815 | |
| 2437 | GUGUCUAUC ACUGCAAA | 397 | UUUGCAGU CUGAUGAGGCCGUUAGGCCCGAA AUAGACAC | 9816 | |
| 2465 | GAAGGGCUC UGUGGAAA | 398 | UUUCCACA CUCAUGAGGCCGUUAGGCCCGAA AGCCCUUC | 9817 | |
| 2476 | UGGAAAGUU CAGCAUAC | 399 | GUAUGCUG CUGAUGAGGCCGUUAGGCCCGAA ACUUUCCA | 9818 | |
| 2477 | GGAAAGUUC AGCAUACC | 400 | GGUAUGCU CUGAUGAGGCCGUUAGGCCCGAA AACUUUCC | 9819 | |
| 2483 | UUCAGCAUA CCUCACUG | 401 | CAGUGAGC CUGAUGAGGCCGUUAGGCCCGAA AUGCUGAA | 9820 | |
| 2487 | GCAUACCUC ACUGUUCA | 402 | UGAACAGU CUGAUGAGGCCGUUAGGCCCGAA AGGUAUCC | 9821 | |
| 2493 | CUCACUGUU CAAGGAAC | 403 | GUUCCUUG CUGAUGAGGCCGUUAGGCCCGAA ACAGUGAG | 9822 | |
| 2494 | UCACUGUUC AAGGAACC | 404 | GGUUCCUU CUGAUGAGGCCGUUAGGCCCGAA AACAGUGA | 9823 | |
| 2504 | AGGAACCUC GGACAAGU | 405 | ACUUGUCC CUGAUGAGGCCGUUAGGCCCGAA AGGUUCCU | 9824 | |
| 2513 | GGACAAGUC UAAUCUGG | 406 | CCAGAUUA CUGAUGAGGCCGUUAGGCCCGAA ACUUGUCC | 9825 | |
| 2515 | ACAAGUCUA AUCUGGAG | 407 | CUCCAGAU CUGAUGAGGCCGUUAGGCCCGAA AGACUUGU | 9826 | |
| 2518 | AGUCUAAUC UGGACCUG | 408 | CAGCUCCA CUGAUGAGGCCGUUAGGCCCGAA AUUAGACU | 9827 | |
| 2529 | GAGCUGAUC ACUCUAAC | 409 | GUUAGAGU CUGAUGAGGCCGUUAGGCCCGAA AUCAUCUC | 9828 | |
| 2533 | UGAUCACUC UAACAUGC | 410 | GCAUGUUA CUGAUGAGGCCGUUAGGCCCGAA AGUGAUCA | 9829 | |
| 2535 | AUCACUCUA ACAUGCAC | 411 | GUGCAUGU CUGAUGAGGCCGUUAGGCCCGAA AGAGUGAU | 9830 | |
| 2560 | CUGCGACUC UCUUCUCG | 412 | CCAGAAGA CUGAUGAGGCCGUUAGGCCCGAA AGUCGCAG | 9831 | |
| 2562 | GCGACUCUC UUCUGGCU | 413 | AGCCAGAA CUGAUGAGGCCGUUAGGCCCGAA AGAGUCGC | 9832 | |
| 2564 | GACUCUCUU CUGGCUCC | 414 | GGAGCCAG CUGAUGAGGCCGUUAGGCCCGAA AGAGAGUC | 9833 | |
| 2565 | ACUCUCUUC UGGCUCCU | 415 | AGGAGCCA CUGAUGAGGCCGUUAGGCCCGAA AAGAGAGU | 9834 | |
| 2571 | UUCUGGCUC CUAUUAAC | 416 | GUUAAUAG CUGAUGAGGCCGUUAGGCCCGAA AGCCAGAA | 9835 | |
| 2574 | UGGCUCCUA UUAACCCU | 417 | AGGGUUAA CUGAUGAGGCCGUUAGGCCCGAA AGGAGCCA | 9836 | |
| 2576 | GCUCCUAUU AACCCUCC | 418 | GGAGGGUU CUGAUGAGGCCGUUAGGCCCGAA AUAGGAGC | 9837 | |
| 2577 | CUCCUAUUA ACCCUCCU | 419 | AGGAGGGU CUGAUGAGGCCGUUAGGCCCGAA AAUAGGAG | 9838 | |
| 2583 | UUAACCCUC CUUAUCCG | 420 | CGGAUAAG CUGAUGAGGCCGUUAGGCCCGAA AGGGUUAA | 9839 | |
| 2586 | ACCCUCCUU AUCCGAAA | 421 | UUUCGGAU CUGAUGAGGCCGUUAGGCCCGAA AGGAGGGU | 9840 | |
| 2587 | CCCUCCUUA UCCGAAAA | 422 | UUUUCGGA CUGAUGAGGCCGUUAGGCCCGAA AAGGAGGG | 9841 | |
| 2589 | CUCCUUAUC CGAAAAAU | 423 | AUUUUUCG CUGAUGAGGCCGUUAGGCCCGAA AUAAGGAG | 9842 | |
| 2606 | GAAAAGGUC UUCUUCUG | 424 | CAGAAGAA CUGAUGAGGCCGUUAGGCCCGAA ACCUUUUC | 9843 | |
| 2608 | AAAGGUCUU CUUCUGAA | 425 | UUCAGAAG CUGAUGAGGCCGUUAGGCCCGAA AGACCUUU | 9844 | |
| 2609 | AAGGUCUUC UUCUGAAA | 426 | UUUCAGAA CUGAUGAGGCCGUUAGGCCCGAA AAGACCUU | 9845 | |
| 2611 | GGUCUUCUU CUGAAAUA | 427 | UAUUUCAG CUGAUGAGGCCGUUAGGCCCGAA AGAAGACC | 9846 | |
| 2612 | GUCUUCUUC UGAAAUAA | 428 | UUAUUUCA CUGAUGAGGCCGUUAGGCCCGAA AAGAAGAC | 9847 | |
| 2619 | UCUGAAAUA AAGACUGA | 429 | UCAGUCUU CUGAUGAGGCCGUUAGGCCCGAA AUUUCAGA | 9848 | |
| 2630 | GACUGACUA CCUAUCAA | 430 | UUGAUAGG CUGAUGAGGCCGUUAGGCCCGAA AGUCAGUC | 9849 | |
| 2634 | GACUACCUA UCAAUUAU | 431 | AUAAUUGA CUGAUGAGGCCGUUAGGCCCGAA AGGUAGUC | 9850 | |
| 2636 | CUACCUAUC AAUUAUAA | 432 | UUAUAAUU CUGAUGAGGCCGUUAGGCCCGAA AUAGGUAG | 9851 | |
| 2640 | CUAUCAAUU AUAAUGGA | 433 | UCCAUUAU CUGAUGAGGCCGUUAGGCCCGAA AUUGAUAG | 9852 | |
| 2641 | UAUCAAUUA UAAUGGAC | 434 | GUCCAUUA CUGAUGAGGCCGUUAGGCCCGAA AAUUGAUA | 9853 | |
| 2643 | UCAAUUAUA AUGGACCC | 435 | GGGUCCAU CUGAUGAGGCCGUUAGGCCCGAA AUAAUUGA | 9854 | |
| 2661 | GAUGAAGUU CCUUUGGA | 436 | UCCAAAGG CUGAUGAGGCCGUUAGGCCCGAA ACUUCAUC | 9855 | |
| 2662 | AUGAAGUUC CUUUGGAU | 437 | AUCCAAAG CUGAUGAGGCCGUUAGGCCCGAA AACUUCAU | 9856 | |
| 2665 | AAGUUCCUU UGGAUGAG | 438 | CUCAUCCA CUGAUGAGGCCGUUAGGCCCGAA AGGAACUU | 9857 | |
| 2666 | AGUUCCUUU GGAUGAGC | 439 | GCUCAUCC CUGAUGAGGCCGUUAGGCCCGAA AAGGAACU | 9858 | |
| 2688 | GAGCGGCUC CCUUAUGA | 440 | UCAUAAGG CUGAUGAGGCCGUUAGGCCCGAA AGCCGCUC | 9859 | |
| 2692 | GGCUCCCUU AUGAUGCC | 441 | GGCAUCAU CUGAUGAGGCCGUUAGGCCCGAA AGGGAGCC | 9860 | |
| 2693 | GCUCCCUUA UGAUGCCA | 442 | UGGCAUCA CUGAUGAGGCCGUUAGGCCCGAA AAGGGAGC | 9861 | |
| 2714 | GUGGGAGUU UGCCCGGG | 443 | CCCGGGCA CUGAUGAGGCCGUUAGGCCCGAA ACUCCCAC | 9862 | |
| 2715 | UGGGAGUUU GCCCGGGA | 444 | UCCCGGGC CUGAUGAGGCCGUUAGGCCCGAA AACUCCCA | 9863 | |
| 2730 | GAGAGACUU AAACUCGG | 445 | CCCAGUUU CUGAUGAGGCCGUUAGGCCCGAA AGUCUCUC | 9864 | |
| 2731 | AGAGACUUA AACUGGGC | 446 | GCCCAGUU CUGAUGAGGCCGUUAGGCCCGAA AAGUCUCU | 9865 | |
| 2744 | GGGCAAAUC ACUUGGAA | 447 | UUCCAAGU CUGAUGAGGCCGUUAGGCCCGAA AUUUGCCC | 9866 | |
| 2748 | AAAUCACUU GGAAGAGC | 448 | CCUCUUCC CUGAUGAGGCCGUUAGGCCCGAA AGUGAUUU | 9867 | |
| 2761 | GAGGGGCUU UUGGAAAA | 449 | UUUUCCAA CUGAUGAGGCCGUUAGGCCCGAA AGCCCCUC | 9868 | |
| 2762 | AGGGGCUUU UGGAAAAG | 450 | CUUUUCCA CUGAUGAGGCCGUUAGGCCCGAA AAGCCCCU | 9869 | |
| 2763 | GGGGCUUUU GGAAAAGU | 451 | ACUUUUCC CUGAUGAGGCCGUUAGGCCCGAA AAAGCCCC | 9870 | |
| 2775 | AAAGUGGUU CAAGCAUC | 452 | GAUGCUUG CUGAUGAGGCCGUUAGGCCCGAA ACCACUUU | 9871 | |
| 2776 | AAGUGGUUC AAGCAUCA | 453 | UGAUGCUU CUGAUGAGGCCGUUAGGCCCGAA AACCACUU | 9872 | |
| 2783 | UCAAGCAUC AGCAUUUG | 454 | CAAAUGCU CUCAUGAGGCCGUUAGGCCCGAA AUGCUUGA | 9873 | |
| 2789 | AUCAGCAUU UGGCAUUA | 455 | UAAUGCCA CUGAUGAGGCCGUUAGGCCCGAA AUGCUGAU | 9874 | |
| 2790 | UCAGCAUUU GGCAUUAA | 456 | UUAAUGCC CUGAUGAGGCCGUUAGGCCCGAA AAUGCUGA | 9875 | |
| 2796 | UUUGGCAUU AAGAAAUC | 457 | GAUUUCUU CUGAUGAGGCCGUUAGGCCCGAA AUGCCAAA | 9876 | |
| 2797 | UUGGCAUUA AGAAAUCA | 458 | UGAUUUCU CUGAUGAGGCCGUUAGGCCCGAA AAUGCCAA | 9877 | |
| 2804 | UAAGAAAUC ACCUACGU | 459 | AGGUAGOG CUGAUGAGGCCGUUAGGCCCGAA AUUUCUUA | 9878 | |
| 2809 | AAUCACCUA CGUGCCGG | 460 | CCGGCACG CUGAUGAGGCCGUUAGGCCCGAA AGGUGAUU | 9879 | |
| 2864 | CAGCGAGUA CAAAGCUC | 461 | GAGCUUUG CUGAUGAGGCCGUUAGGCCCGAA ACUCGCUG | 9880 | |
| 2872 | ACAAAGCUC UGAUGACU | 462 | AGUCAUCA CUGAUGAGGCCGUUAGGCCCGAA AGCUUUGU | 9881 | |
| 2886 | ACUGAGCUA AAAAUCUU | 463 | AAGAUUUU CUGAUGAGGCCGUUAGGCCCGAA AGCUCAGU | 9882 | |
| 2892 | CUAAAAAUC UUGACCCA | 464 | UGGGUCAA CUGAUGAGGCCGUUAGGCCCGAA AUUUUUAG | 9883 | |
| 2894 | AAPAAUCUU GACCCACA | 465 | UGUGGGUC CUGAUGAGGCCGUUAGGCCCGAA AGAUUUUU | 9884 | |
| 2904 | ACCCACAUU GGCCACCA | 466 | UGGUGGCC CUGAUGAGGCCGUUAGGCCCGAA AUGUGGGU | 9885 | |
| 2914 | GCCACCAUC UGAACGUG | 467 | CACGUUCA CUGAUGAGGCCGUUAGGCCCGAA AUGGUGGC | 9886 | |
| 2925 | AACGUGGUU AACCUGCU | 468 | AGCAGGUU CUGAUGAGGCCGUUAGGCCCGAA ACCACGUU | 9887 | |
| 2926 | ACGUGGUUA ACCUGCUG | 469 | CAGCAGGU CUGAUGAGGCCGUUAGGCCCGAA AACCACGU | 9888 | |
| 2962 | GAGGGCCUC UGAUGGUG | 470 | CACCAUCA CUGAUGAGGCCGUUAGGCCCGAA AGGCCCUC | 9889 | |
| 2973 | AUGGUGAUU GUUGAAUA | 471 | UAUUCAAC CUGAUGAGGCCGUUAGGCCCGAA AUCACCAU | 9890 | |
| 2976 | GUGAUUGUU GAAUACUG | 472 | CAGUAUUC CUGAUGAGGCCGUUAGGCCCGAA ACAAUCAC | 9891 | |
| 2981 | UGUUGAAUA CUGCAAAU | 473 | AUUUGCAG CUGAUGAGGCCGUUAGGCCCGAA AUUCAACA | 9892 | |
| 2990 | CUGCAAAUA UGGAAAUC | 474 | GAUUUCCA CUGAUGAGGCCGUUAGGCCCGAA AUUUGCAG | 9893 | |
| 2998 | AUGGAAAUC UCUCCAAC | 475 | GUUGGAGA CUGAUGAGGCCGUUAGGCCCGAA AUUUCCAU | 9894 | |
| 3000 | GGAAAUCUC UCCAACUA | 476 | UAGUUGGA CUGAUGAGGCCGUUAGGCCCGAA AGAUUUCC | 9895 | |
| 3002 | AAAUCUCUC CAACUACC | 477 | GGUAGUUG CUGAUGAGGCCGUUAGGCCCGAA AGAGAUUU | 9896 | |
| 3008 | CUCCAACUA CCUCAAGA | 478 | UCUUGAGG CUGAUGAGGCCGUUAGGCCCGAA AGGUGGAG | 9897 | |
| 3012 | AACUACCUC AAGAGCAA | 479 | UUGCUCUU CUGAUGAGGCCGUUAGGCCCGAA AGGUAGUU | 9898 | |
| 3029 | ACGUGACUU AUUUUUUC | 480 | GAAAAAAU CUGAUGAGGCCGUUAGGCCCGAA AGUCACGU | 9899 | |
| 3030 | CGUGACUUA UUUUUUCU | 481 | AGAAAAAA CUGAUGAGGCCGUUAGGCCCGAA AAGUCACG | 9900 | |
| 3032 | UGACUUAUU UUUUCUCA | 482 | UGAGAAAA CUGAUGAGGCCGUUAGGCCCGAA AUAAGUCA | 9901 | |
| 3033 | GACUUAUUU UUUCUCAA | 483 | UUGAGAAA CUGAUGAGGCCGUUAGGCCCGAA AAUAAGUC | 9902 | |
| 3034 | ACUUAUUUU UUCUCAAC | 484 | GUUGAGAA CUGAUGAGGCCGUUAGGCCCGAA AAAUAAGU | 9903 | |
| 3035 | CUUAUUUUU UCUCAACA | 485 | UGUUGAGA CUGAUGAGGCCGUUAGGCCCGAA AAAAUAAG | 9904 | |
| 3036 | UUAUUUUUU CUCAACAA | 486 | UUGUUGAG CUGAUGAGGCCGUUAGGCCCGAA AAAAAUAA | 9905 | |
| 3037 | UAUUUUUUC UCAACAAG | 487 | CUUGUUGA CUGAUGAGGCCGUUAGGCCCGAA AAAAAAUA | 9906 | |
| 3039 | UUUUUUCUC AACAAGGA | 488 | UCCUUGUU CUGAUGAGGCCGUUAGGCCCGAA AGAAAAAA | 9907 | |
| 3057 | GCAGCACUA CACAUGGA | 489 | UCCAUGUG CUGAUGAGGCCGUUAGGCCCGAA AGUGCUGC | 9908 | |
| 3070 | UGGAGCCUA AGAAAGAA | 490 | UUCUUUCU CUGAUGAGGCCGUUAGGCCCGAA AGOCUCCA | 9909 | |
| 3120 | CCAAGACUA GAUAGCGU | 491 | ACGCUAUC CUGAUGAGGCCGUUAGGCCCGAA AGUCUUGG | 9910 | |
| 3124 | GACUAGAUA GCGUCACC | 492 | GGUGACGC CUGAUGAGGCCGUUAGGCCCGAA AUCUAGUC | 9911 | |
| 3129 | GAUAGCGUC ACCACCAG | 493 | CUGCUGGU CUGAUGAGGCCGUUAGGCCCGAA ACGCUAUC | 9912 | |
| 3146 | CGAAAGCUU UGCGAGCU | 494 | AGCUCGCA CUGAUGAGGCCGUUAGGCCCGAA AGCUUUCG | 9913 | |
| 3147 | GAAAGCUUU GCGAGCUC | 495 | GAGCUCGC CUGAUGAGGCCGUUAGGCCCGAA AAGCUUUC | 9914 | |
| 3155 | UGCGAGCUC CGGCUUUC | 496 | GAAAGCCG CUGAUGAGGCCGUUAGGCCCGAA AGCUCGCA | 9915 | |
| 3161 | CUCCGGCUU UCAGGAAG | 497 | CUUCCUGA CUGAUGAGGCCGUUAGGCCCGAA AGCCGGAG | 9916 | |
| 3162 | UCCGGCTUU CAGGAAGA | 498 | UCUUCCUG CUGAUGAGGCCGUUAGGCCCGAA AAGCCGGA | 9917 | |
| 3163 | CCGGCUUUC AGGAAGAU | 499 | AUCUUCCU CUGAUGAGGCCGUUAGGCCCGAA AAAGCCGG | 9918 | |
| 3172 | AGGAAGAUA AAACUCUG | 500 | CAGACUUU CUGAUGAGGCCGUUAGGCCCGAA AUCUUCCU | 9919 | |
| 3178 | AUAAAAGUC UGAGUGAU | 501 | AUCACUCA CUGAUGAGGCCGUUAGGCCCGAA ACUUUUAU | 9920 | |
| 3189 | AGUGAUGUU GAGGAAGA | 502 | UCUUCCUC CUGAUGAGGCCGUUAGGCCCGAA ACAUCACU | 9921 | |
| 3205 | AGGAGGAUU CUGACCGU | 503 | ACCGUCAG CUGAUGAGGCCGUUAGGCCCGAA AUCCUCCU | 9922 | |
| 3206 | CUAGGACUC UGACGGUU | 504 | AACCGUCA CUGAUGAGGCCGUUAGGCCCGAA AAUCCUCC | 9923 | |
| 3214 | CUGACGGUU UCUACAAG | 505 | CUUGUAGA CUGAUGAGGCCGUUAGGCCCGAA ACCGUCAG | 9924 | |
| 3215 | UGACGGUUU CUACAAGG | 506 | CCUUGUAG CUGAUGAGGCCGUUAGGCCCGAA AACCGUCA | 9925 | |
| 3216 | GACGGUUUC UACAAGGA | 507 | UCCUUGUA CUGAUGAGGCCGUUAGGCCCGAA AAACCGUC | 9926 | |
| 3218 | CGGUUUCUA CAAGGAGC | 508 | GCUCCUUG CUGAUGAGGCCGUUAGGCCCGAA AGAAACCG | 9927 | |
| 3231 | GAGCCCAUC ACUAUGGA | 509 | UCCAUAGU CUGAUGAGGCCGUUAGGCCCGAA AUGGGCUC | 9928 | |
| 3235 | CCAUCACUA UGGAAGAU | 510 | AUCUUCCA CUGAUGAGGCCGUUAGGCCCGAA AGUGAUGG | 9929 | |
| 3244 | UGGAAGAUC UGAUUUCU | 511 | AGAAAUCA CUGAUGAGGCCGUUAGGCCCGAA AUCUUCCA | 9930 | |
| 3249 | GAUCUGAUU UCUUACAG | 512 | CUGUAAGA CUGAUGAGGCCGUUAGGCCCGAA AUCAGAUC | 9931 | |
| 3250 | AUCUGAUUU CUUACAGU | 513 | ACUGUAAG CUGAUGAGGCCGUUAGGCCCGAA AAUCAGAU | 9932 | |
| 3251 | UCUGAUUUC UUACAGUU | 514 | AACUGUAA CUGAUGAGGCCGUUAGGCCCGAA AAAUCAGA | 9933 | |
| 3253 | UGAUUUCUU ACAGUUUU | 515 | AAAACUGU CUGAUGAGGCCGUUAGGCCCGAA AGAAAUCA | 9934 | |
| 3254 | GAUUUCUUA CAGUUUUC | 516 | GAAAACUG CUGAUGAGGCCGUUAGGCCCGAA AAGAAAUC | 9935 | |
| 3259 | CUUACAGUU UUCAAGUG | 517 | CACUUGAA CUGAUGAGGCCGUUAGGCCCGAA ACUGUAAG | 9936 | |
| 3260 | UUACAGUUU UCAAGUGG | 518 | CCACUUGA CUGAUGAGGCCGUUAGGCCCGAA AACUGUAA | 9937 | |
| 3261 | UACAGUUUU CAAGUGGC | 519 | GCCACUUG CUGAUGAGGCCGUUAGGCCCGAA AAACUGUA | 9938 | |
| 3262 | ACAGUUUUC AAGUGGCC | 520 | GGCCACUU CUGAUGAGGCCGUUAGGCCCGAA AAAACUGU | 9939 | |
| 3284 | CAUGGAGUU CCUGUCUU | 521 | AAGACAGG CUGAUGAGGCCGUUAGGCCCGAA ACUCCAUG | 9940 | |
| 3285 | AUGGAGUUC CUGUCUUC | 522 | GAAGACAG CUGAUGAGGCCGUUAGGCCCGAA AACUCCAU | 9941 | |
| 3290 | GUUCCUGUC UUCCAGAA | 523 | UUCUGGAA CUGAUGAGGCCGUUAGGCCCGAA ACAGGAAC | 9942 | |
| 3292 | UCCUGUCUU CCAGAAAG | 524 | CUUUCUGG CUGAUGAGGCCGUUAGGCCCGAA AGACAGGA | 9943 | |
| 3293 | CCUGUCUUC CAGAAAGU | 525 | ACUUUCUG CUGAUGAGGCCGUUAGGCCCGAA AAGACAGG | 9944 | |
| 3306 | AAGUGCAUU CAUCGGGA | 526 | UCCCGAUG CUGAUGAGGCCGUUAGGCCCGAA AUGCACUU | 9945 | |
| 3307 | AGUGCAUUC AUCGGGAC | 527 | GUCCCGAU CUGAUGAGGCCGUUAGGCCCGAA AAUGCACU | 9946 | |
| 3310 | GCAUUCAUC GGGACCUG | 528 | CAGGUCCC CUGAUGAGGCCGUUAGGCCCGAA AUGAAUGC | 9947 | |
| 3333 | AGAAACAUU CUGUGAUC | 529 | GAUAAAAG CUGAUGAGGCCGUUAGGCCCGAA AUGUUUCU | 9948 | |
| 3334 | GAAACAUUC UUUUAUCU | 530 | AGAUAAAA CUGAUGAGGCCGUUAGGCCCGAA AAUGUUUC | 9949 | |
| 3336 | AACAUUCUU UUAUCUGA | 531 | UCAGAUAA CUGAUGAGGCCGUUAGGCCCGAA AGAAUGUU | 9950 | |
| 3337 | ACAUUCUUU UAUCUGAG | 532 | CUCAGAUA CUGAUGAGGCCGUUAGGCCCGAA AAGAAUGU | 9951 | |
| 3338 | CAUUCUUUU AUCUGAGA | 533 | UCUCAGAU CUGAUGAGGCCGUUAGGCCCGAA AAAGAAUG | 9952 | |
| 3339 | AGUCUUUUA UCUGAGAA | 534 | GUCUCAGA CUGAUGAGGCCGUUAGGCCCGAA AAAAGAAU | 9953 | |
| 3341 | UCUUUUAUC UGAGAACA | 535 | UGGUCUCA CUGAUGAGGCCGUUAGGCCCGAA AUAAAAGA | 9954 | |
| 3363 | GUGAAGAUU UGUGAUUU | 536 | AAAUCACA CUGAUGAGGCCGUUAGGCCCGAA AUCUUCAC | 9955 | |
| 3364 | UGAAGAUUU GUGAUUUU | 537 | AAAAUCAC CUGAUGAGGCCGUUAGGCCCGAA AAUCUUCA | 9956 | |
| 3370 | UUUGUGAUU UUGGCCUU | 538 | AAGGCCAA CUGAUGAGGCCGUUAGGCCCGAA AUCACAAA | 9957 | |
| 3371 | UUGUGAUUU UGGCCUUG | 539 | CAAGGCCA CUGAUGAGGCCGUUAGGCCCGAA AAUCACAA | 9958 | |
| 3372 | UGUGAUUUU GGCCUUGC | 540 | GCAAGGCC CUGAUGAGGCCGUUAGGCCCGAA AAAUCACA | 9959 | |
| 3378 | UUUGGCCUU GCCCGGGA | 541 | UCCCGGGC CUGAUGAGGCCGUUAGGCCCGAA AGGCCAAA | 9960 | |
| 3388 | CCCGGGAUA UUUAUAAG | 542 | CUUAUAAA CUGAUGAGGCCGUUAGGCCCGAA AUCCCGGG | 9961 | |
| 3390 | CGGGAUAUU UAUAAGAA | 543 | UUCUUAUA CUGAUGAGGCCGUUAGGCCCGAA AUAUCCCG | 9962 | |
| 3391 | GGGAUAUUU AUAAGAAC | 544 | GUUCUUAU CUGAUCAGGCCGUUAGGCCCGAA AAUAUCCC | 9963 | |
| 3392 | GGAUAUUUA UAAGAACC | 545 | GGUUCUUA CUGAUGAGGCCGUUAGGCCCGAA AAAUAUCC | 9964 | |
| 3394 | AUAUUUAUA AGAACCCC | 546 | GGGGUUCU CUGAUGAGGCCGUUAGGCCCGAA AUAAAUAU | 9965 | |
| 3406 | ACCCCGAUU AUGUGAGA | 547 | UCUCACAU CUGAUGAGGCCGUUAGGCCCGAA AUCGGGGU | 9966 | |
| 3407 | CCCCGAUUA UGUGAGAA | 548 | UUCUCACA CUGAUGAGGCCGUUAGGCCCGAA AAUCGGGG | 9967 | |
| 3424 | AAGGAGAUA CUGGACUU | 549 | AAGUCGAG CUGAUGAGGCCGUUAGGCCCGAA AUCUCCUU | 9968 | |
| 3427 | GAGAUACUC GACUUCCU | 550 | AGGAAGUC CUGAUGAGGCCGUUAGGCCCGAA AGUAUCUC | 9969 | |
| 3432 | ACUCGACUU CCUCUGAA | 551 | UUCAGAGG CUGAUGAGGCCGUUAGGCCCGAA AGUCGAGU | 9970 | |
| 3433 | CUCGACUUC CUCUGAAA | 552 | UUUCAGAG CUGAUGAGGCCGUUAGGCCCGAA AAGUCGAG | 9971 | |
| 3436 | GACUUCCUC UGAAAUGG | 553 | CCAUUUCA CUGAUGAGGCCGUUAGGCCCGAA AGGAAGUC | 9972 | |
| 3451 | GGAUGGCUC CCGAAUCU | 554 | AGAUUCGG CUGAUGAGGCCGUUAGGCCCGAA AGCCAUCC | 9973 | |
| 3458 | UCCCGAAUC UAUCUUUG | 555 | CAAAGAUA CUGAUGAGGCCGUUAGGCCCGAA AUUCGGGA | 9974 | |
| 3460 | CCGAAUCUA UCUUUGAC | 556 | GUCAAAGA CUGAUGAGGCCGUUAGGCCCGAA AGAUUCGG | 9975 | |
| 3462 | GAAUCUAUC UUUGACAA | 557 | UUGUCAAA CUGAUGAGGCCGUUAGGCCCGAA AUAGAUUC | 9976 | |
| 3464 | AUCUAUCUU UGACAAAA | 558 | UUUUGUCA CUGAUGAGGCCGUUAGGCCCGAA AGAUAGAU | 9977 | |
| 3465 | UCUAUCUUU GACAAAAU | 559 | AUUUUGUC CUGAUGAGGCCGUUAGGCCCGAA AAGAUAGA | 9978 | |
| 3474 | GACAAAAUC UACAGCAC | 560 | GUGCUGUA CUGAUGAGGCCGUUAGGCCCGAA AUUUUGUC | 9979 | |
| 3476 | CAAAAUCUA CAUCACCA | 561 | UGGUGCUG CUGAUGAGGCCGUUAGGCCCGAA AGAUUUUG | 9980 | |
| 3500 | UGUGUGGUC UUACGGAG | 562 | CUCCGUAA CUGAUGAGGCCGUUAGGCCCGAA ACCACACG | 9981 | |
| 3502 | UGUGGUCUU ACGGAGUA | 563 | UACUCCGU CUGAUGAGGCCGUUAGGCCCGAA AGACCACA | 9982 | |
| 3503 | GUGGUCUUA CGGAGUAU | 564 | AUACUCCG CUGAUGAGGCCGUUAGGCCCGAA AAGACCAC | 9983 | |
| 3510 | UACGGAGUA UUGCUGUG | 565 | CACAGCAA CUGAUGAGGCCGUUAGGCCCGAA ACUCCGUA | 9984 | |
| 3512 | CGGAGUAUU GCUGUGGG | 566 | CCCACAGC CUGAUGAGGCCGUUAGGCCCGAA AUACUCCG | 9985 | |
| 3525 | UGGGAAAUC UUCUCCUU | 567 | AAGGAGAA CUGAUGAGGCCGUUAGGCCCGAA AUUUCCCA | 9986 | |
| 3527 | GGAAAUCUU CUCCUUAG | 568 | CUAAGGAG CUGAUGAGGCCGUUAGGCCCGAA AGAUUUCC | 9987 | |
| 3528 | GAAAUCUUC UCCUUAGG | 569 | CCUAAGGA CUGAUGAGGCCGUUAGGCCCGAA AAGAUUUC | 9988 | |
| 3530 | AAUCUUCUC CUUAGGUG | 570 | CACCUAAG CUGAUGAGGCCGUUAGGCCCGAA AGAAGAUU | 9989 | |
| 3533 | CUUCUCCUU AGGUGGGU | 571 | ACCCACCU CUGAUGAGGCCGUUAGGCCCGAA AGGAGAAG | 9990 | |
| 3534 | UUCUCCUUA GGUGGGUC | 572 | GACCCACC CUGAUGAGGCCGUUAGGCCCGAA AAGGAGAA | 9991 | |
| 3542 | AGGUOGGUC UCCAUACC | 573 | GGUAUGGA CUGAUGAGGCCGUUAGGCCCGAA ACCCACCU | 9992 | |
| 3544 | GUGGGUCUC CAUACCCA | 574 | UGGGUAUG CUGAUGAGGCCGUUAGGCCCGAA AGACCCAC | 9993 | |
| 3548 | GUCUCCAUA CCCAGGAG | 575 | CUCCUGGG CUGAUGAGGCCGUUAGGCCCGAA AUGGAGAC | 9994 | |
| 3558 | CCAGGAGUA CAAAUGGA | 576 | UCCAUUUG CUGAUGAGGCCGUUAGGCCCGAA ACUCCUGG | 9995 | |
| 3575 | UGAGGACUU UUGCAGUC | 577 | GACUGCAA CUGAUGAGGCCGUUAGGCCCGAA AGUCCUCA | 9996 | |
| 3576 | GAGGACUUU UGCAGUCG | 578 | CGACUGCA CUGAUGAGGCCGUUAGGCCCGAA AAGUCCUC | 9997 | |
| 3577 | AGGACUUUU GCAGUCGC | 579 | GCGACUGC CUGAUGAGGCCGUUAGGCCCGAA AAAGUCCU | 9998 | |
| 3583 | UUUCCAGUC GCCUGAGG | 580 | CCUCAGGC CUGAUGAGGCCGUUAGGCCCGAA ACUGCAAA | 9999 | |
| 3613 | UGAGAGCUC CUGAGUAC | 581 | GUACUCAG CUGAUGAGGCCGUUAGGCCCGAA AGCUCUCA | 10000 | |
| 3620 | UCCUGAGUA CUCUACUC | 582 | GAGUAGAG CUGAUGAGGCCGUUAGGCCCGAA ACUCAGGA | 10001 | |
| 3623 | UGAGUACUC UACUCCUG | 583 | CAGGAGUA CUGAUGAGGCCGUUAGGCCCGAA AGUACUCA | 10002 | |
| 3625 | AGUACUCUA CUCCUGAA | 584 | UUCAGGAG CUGAUGAGGCCGUUAGGCCCGAA AGAGUACU | 10003 | |
| 3628 | ACUCUACUC CUGAAAUC | 585 | GAUUUCAG CUGAUGAGGCCGUUAGGCCCGAA AGUAGAGU | 10004 | |
| 3636 | CCUGAAAUC UAUCAGAU | 586 | AUCUGAUA CUGAUGAGGCCGUUAGGCCCGAA AUUUCAGG | 10005 | |
| 3638 | UGAAAUCUA UCAGAUCA | 587 | UGAUCUGA CUGAUGAGGCCGUUAGGCCCGAA AGAUUUCA | 10006 | |
| 3640 | AAAUCUAUC AGAUCAUG | 588 | CAUGAUCU CUGAUGAGGCCGUUAGGCCCGAA AUAGAUUU | 10007 | |
| 3645 | UAUCAGAUC AUGCUGGA | 589 | UCCAGCAU CUGAUGAGGCCGUUAGGCCCGAA AUCUGAUA | 10008 | |
| 3689 | GCCAAGAUU UGCAGAAC | 590 | GUUCUGCA CUGAUGAGGCCGUUAGGCCCGAA AUCUUGGC | 10009 | |
| 3690 | CCAAGAUUU GCAGAACU | 591 | AGUUCUGC CUGAUGAGGCCGUUAGGCCCGAA AAUCUUGG | 10010 | |
| 3699 | GCAGAACUU GUGGAAAA | 592 | UUUUCCAC CUGAUGAGGCCGUUAGGCCCGAA AGUUCUGC | 10011 | |
| 3711 | GAAAAACUA GGUGAUUU | 593 | AAAUCACC CUGAUGAGGCCGUUAGGCCCGAA AGUUUUUC | 10012 | |
| 3718 | UAGGUGAUU UGCUUCAA | 594 | UUGAAGCA CUGAUGAGGCCGUUAGGCCCGAA AUCACCUA | 10013 | |
| 3719 | AGGUGAUUU GCUUCAAG | 595 | CUUGAAGC CUGAUGAGGCCGUUAGGCCCGAA AAUCACCU | 10014 | |
| 3723 | GAUUUGCUU CAAGCAAA | 596 | UUUGCUUG CUGAUGAGGCCGUUAGGCCCGAA AGCAAAUC | 10015 | |
| 3724 | AUUUGCUUC AAGCAAAU | 597 | AUUUGCUU CUGAUGAGGCCGUUAGGCCCGAA AAGCAAAU | 10016 | |
| 3735 | GCAAAUGUA CAACAGGA | 598 | UCCUGUUG CUGAUCAGGCCGUUAGGCCCGAA ACAUUUGC | 10017 | |
| 3748 | AGGAUGGUA AAGACUAC | 599 | GUAGUCUU CUGAUGAGGCCGUUAGGCCCGAA ACCAUCCU | 10018 | |
| 3755 | UAAAGACUA CAUCCCAA | 600 | UUGGGAUG CUGAUGAGGCCGUUAGGCCCGAA AGUCUUUA | 10019 | |
| 3759 | GACUACAUC CCAAUCAA | 601 | UUGAUUGG CUGAUGAGGCCGUUAGGCCCGAA AUGUAGUC | 10020 | |
| 3765 | AUCCCAAUC AAUGCCAU | 602 | AUGGCAUU CUGAUGAGGCCGUUAGGCCCGAA AGUGOGAG | 10021 | |
| 3774 | AAUGCCAUA CUGACAGG | 603 | CCUGUCAG CUGAUGAGGCCGUUAGGCCCGAA AUGGCAUU | 10022 | |
| 3787 | CAGGAAAUA GUGGGUUU | 604 | AAACCCAC CUGAUGAGGCCGUUAGGCCCGAA AUUUCCUG | 10023 | |
| 3794 | UAGUGGGUU UACAUACU | 605 | AGUAUGUA CUGAUGAGGCCGUUAGGCCCGAA ACCCACUA | 10024 | |
| 3795 | AGUGGGUUU ACAUACUC | 606 | GAGUAUGU CUGAUGAGGCCGUUAGGCCCGAA AACCCACU | 10025 | |
| 3796 | GUGGGUUUA CAUACUCA | 607 | UGAGUAUG CUGAUGAGGCCGUUAGGCCCGAA AAACCCAC | 10026 | |
| 3800 | GUUUACAUA CUCAACUC | 608 | GAGUUGAG CUGAUGAGGCCGUUAGGCCCGAA AUGUAAAC | 10027 | |
| 3803 | UACAUACUC AACUCCUG | 609 | CAGGAGUU CUGAUGAGGCCGUUAGGCCCGAA AGUAUGUA | 10028 | |
| 3808 | ACUCAACUC CUGCCUUC | 610 | GAAGGCAG CUGAUGAGGCCGUUAGGCCCGAA AGUUGAGU | 10029 | |
| 3815 | UCCUGCCUU CUCUGAGG | 611 | CCUCAGAG CUGAUGAGGCCGUUAGGCCCGAA AGGCAGGA | 10030 | |
| 3816 | CCUGCCUUC UCUGAGGA | 612 | UCCUCAGA CUGAUGAGGCCGUUAGGCCCGAA AAGGCAGG | 10031 | |
| 3818 | UGCCUUCUC UGAGGACU | 613 | AGUCCUCA CUGAUGAGGCCGUUAGGCCCGAA AGAAGGCA | 10032 | |
| 3827 | UGAGGACUG CUUCAAGG | 614 | CCUUGAAG CUGAUGAGGCCGUUAGGCCCGAA AGUCCUCA | 10033 | |
| 3828 | GAGGACUUC UUCAAGGA | 615 | UCCUUGAA CUGAUGAGGCCGUUAGGCCCGAA AAGUCCUC | 10034 | |
| 3830 | GGACUUCUU CAAGGAAA | 616 | UUUCCUUG CUGAUGAGGCCGUUAGGCCCGAA AGAAGUCC | 10035 | |
| 3831 | GACUUCUUC AAGGAAAG | 617 | CUUUCCUU CUGAUGAGGCCGUUAGGCCCGAA AAGAAGUC | 10036 | |
| 3841 | AGGAAAGUA UUUCAGCU | 618 | AGCUGAAA CUGAUGAGGCCGUUAGGCCCGAA ACUUUCCU | 10037 | |
| 3843 | GAAAGUAUU UCAGCUCC | 619 | GGAGCUGA CUGAUGAGGCCGUUAGGCCCGAA AUACUUUC | 10038 | |
| 3844 | AAAGUAUUU CAGCUCCG | 620 | CGGAGCUG CUGAUGAGGCCGUUAGGCCCGAA AAUACUUU | 10039 | |
| 3845 | AAGUAUUUC AGCUCCGA | 621 | UCGGAGCU CUGAUGAGGCCGUUAGGCCCGAA AAAUACUU | 10040 | |
| 3850 | UUUCAGCUC CGAAGUUU | 622 | AAACUUCG CUGAUGAGGCCGUUAGGCCCGAA AGCUGAAA | 10041 | |
| 3857 | UCCGAAGUU UAAUUCAG | 623 | CUGAAUUA CUGAUGAGGCCGUUAGGCCCGAA ACUUCGGA | 10042 | |
| 3858 | CCGAAGUUU AAUUCAGG | 624 | CCUGAAUU CUGAUGAGGCCGUUAGGCCCGAA AACUUCGG | 10043 | |
| 3859 | CGAAGUUUA AUUCAGGA | 625 | UCCUGAAU CUGAUGAGGCCGUUAGGCCCGAA AAACUUCG | 10044 | |
| 3862 | AGUUUAAUU CAGGAAGC | 626 | GCUUCCUG CUGAUGAGGCCGUUAGGCCCGAA AUUAAACU | 10045 | |
| 3863 | GUUUAAUUC AGGAAGCU | 627 | AGCUUCCU CUGAUGAGGCCGUUAGGCCCGAA AAUUAAAC | 10046 | |
| 3872 | AGGAAGCUC UGAUGAUG | 628 | CAUCAUCA CUGAUGAGGCCGUUAGGCCCGAA AGCUGCCU | 10047 | |
| 3882 | GAUGAUGUC AGAUAUGU | 629 | ACAUAUCU CUGAUGAGGCCGUUAGGCCCGAA ACAUCAUC | 10048 | |
| 3887 | UGUCAGAUA UGUAAAUG | 630 | CAUUUACA CUGAUGAGGCCGUGAGGCCCGAA AUCUGACA | 10049 | |
| 3891 | AGAUAUGUA AAUGCUUU | 631 | AAAGCAUU CUGAUGAGGCCGUUAGGCCCGAA ACAUAUCU | 10050 | |
| 3898 | UAAAUGCUU UCAAGUUC | 632 | GAACUUGA CUGAUGAGGCCGUUAGGCCCGAA AGCAUUUA | 10051 | |
| 3899 | AAAUGCUUU CAAGUUCA | 633 | UGAACUUG CUGAUGAGGCCGUUAGGCCCGAA AAGCAUUU | 10052 | |
| 3900 | AAUGCUUUC AAGUUCAU | 634 | AUGAACUU CUGAUGAGGCCGUUAGGCCCGAA AAAGCAUU | 10053 | |
| 3905 | UUUCAAGUU CAUGAGCC | 635 | GGCUCAUG CUGAUGAGGCCGUUAGGCCCGAA ACUUGAAA | 10054 | |
| 3906 | UUCAAGUUC AUGAGCCU | 636 | AGGCUCAU CUGAUGAGGCCGUUAGGCCCGAA AACUGGAA | 10055 | |
| 3924 | GAAAGAAUC AAAACCUU | 637 | AAGGUUUU CUGAUGAGGCCGUUAGGCCCGAA AUUCUUUC | 10056 | |
| 3932 | CAAAACCUU UGAAGAAC | 638 | GUUCUUCA CUGAUGAGGCCGUUAGGCCCGAA AGGUUGUG | 10057 | |
| 3933 | AAAACCUUG GAAGAACU | 639 | AGUUCUUC CUGAUGAGGCCGUUAGGCCCGAA AAGGUUUU | 10058 | |
| 3942 | GAAGAACUU UUACCGAA | 640 | UUCGGUAA CUGAUGAGGCCGUUAGGCCCGAA AGUUCUUC | 10059 | |
| 3943 | AAGAACUUU UACCGAAU | 641 | AUUCGGUA CUGAUGAGGCCGUUAGGCCCGAA AAGUUCUU | 10060 | |
| 3944 | AGAACUUUU ACCGAAUG | 642 | CAUUCGGU CUGAUGAGGCCGUUAGGCCCGAA AAAGUUCU | 10061 | |
| 3945 | GAACUUUUA CCGAAUGC | 643 | GCAUUCGG CUGAUGAGGCCGUUAGGCCCGAA AAAAGUUC | 10062 | |
| 3959 | UGCCACCUC CAUGUUUG | 644 | CAAACAUG CUGAUGAGGCCGUUAGGCCCGAA AGGUGGCA | 10063 | |
| 3965 | CUCCAUGUU UGAUCACU | 645 | AGUCAUCA CUGAUGAGGCCGUUAGGCCCGAA ACAUGGAG | 10064 | |
| 3966 | UCCAUGUUU GAUGACUA | 646 | UAGUCAUC CUGAUGAGGCCGUUAGGCCCGAA AACAUGGA | 10065 | |
| 3974 | UGAUGACUA CCAGGGCG | 647 | CGCCCUGG CUGAUGAGGCCGUUAGGCCCGAA AGUCAUCA | 10066 | |
| 3994 | GCAGCACUC UGUUGGCC | 648 | GGCCAACA CUGAUGAGGCCGUUAGGCCCGAA AGUGCUGC | 10067 | |
| 3998 | CACUCUGUU GGCCUCUC | 649 | GAGAGGCC CUGAUGAGGCCGUUAGGCCCGAA ACAGAGUG | 10068 | |
| 4004 | GUUGGCCUC UCCCAUGC | 650 | GCAUGGGA CUGAUGAGGCCGUUAGGCCCGAA AGGCCAAC | 10069 | |
| 4006 | UGOCCUCUC CCAUGCUG | 651 | CAGCAUGG CUGAUGAGGCCGUUAGGCCCGAA AGAGGCCA | 10070 | |
| 4022 | GAAGCGCUU CACCUGGA | 652 | UCCAGGUG CUGAUGAGGCCGUUAGGCCCGAA AGCGCUUC | 10071 | |
| 4023 | AAGCGCUUC ACCUGGAC | 653 | GUCCAGGU CUGAUGAGGCCGUUAGGCCCGAA AAGCGCUU | 10072 | |
| 4052 | CAAGGCCUC GCUCAAGA | 654 | UCUUGAGC CUGAUGAGGCCGUUAGGCCCGAA AGGCCUUG | 10073 | |
| 4056 | GCCUCGCUC AAGAUUGA | 655 | UCAAUCUU CUGAUGAGGCCGUUAGGCCCGAA AGCGAGGC | 10074 | |
| 4062 | CUCAAGAUU GACUUGAG | 656 | CUCAAGUC CUGAUGAGGCCGUUAGGCCCGAA AUCUUGAG | 10075 | |
| 4067 | GAUUGACUU GAGAGUAA | 657 | UUACUCUC CUGAUGAGGCCGUUAGGCCCGAA AGUCAAUC | 10076 | |
| 4074 | UUGAGAGUA ACCAGUAA | 658 | UUACUGGU CUGAUGAGGCCGUUAGGCCCGAA ACUCUCAA | 10077 | |
| 4081 | UAACCAGUA AAAGUAAG | 659 | CUUACUUU CUGAUGAGGCCGUUAGGCCCGAA ACUGGUUA | 10078 | |
| 4087 | GUAAAAGUA AGGAGUCG | 660 | CGACUCCU CUGAUGAGGCCGUUAGGCCCGAA ACUUUUAC | 10079 | |
| 4094 | UAAGGAGUC GGGGCUGU | 661 | ACAGCCCC CUGAUGAGGCCGUUAGGCCCGAA ACUCCUUA | 10080 | |
| 4103 | GGGGCUGUC UGAUGUCA | 662 | UGACAUCA CUGAUGAGGCCGUUAGGCCCGAA ACAGCCCC | 10081 | |
| 4110 | UCUGAUGUC AGCAGGCC | 663 | GGCCUGCU CUGAUGAGGCCGUUAGGCCCGAA ACAUCAGA | 10082 | |
| 4123 | GGCCCAGUU UCUGCCAU | 664 | AUGGCAGA CUGAUGAGGCCGUUAGGCCCGAA ACUGGGCC | 10083 | |
| 4124 | GCCCAGUUU CUGCCAUU | 665 | AAUGGCAG CUGAUGAGGCCGUUAGGCCCGAA AACUGGGC | 10084 | |
| 4125 | CCCAGUUUC UGCCAUUC | 666 | GAAUGGCA CUGAUGAGGCCGUUAGGCCCGAA AAACUGGG | 10085 | |
| 4132 | UCUGCCAUU CCAGCUGU | 667 | ACAGCUGG CUGAUGAGGCCGUUAGGCCCGAA AUGGCAGA | 10086 | |
| 4133 | CUGCCAUUC CAGCUGUG | 668 | CACAGCUG CUGAUGAGGCCGUUAGGCCCGAA AAUGGCAG | 10087 | |
| 4149 | GGGCACGUC AGCGAAGG | 669 | CCUUCGCU CUGAUGAGGCCGUUAGGCCCGAA ACGUGCCC | 10088 | |
| 4169 | GCGCAGGUU CACCUACG | 670 | CGUAGGUG CUGAUGAGGCCGUUAGGCCCGAA ACCUGCGC | 10089 | |
| 4170 | CGCAGGUUC ACCUACGA | 671 | UCGUAGGU CUGAUGAGGCCGUUAGGCCCGAA AACCUGCG | 10090 | |
| 4175 | GUUCACCUA CGACCACG | 672 | CGUGGUCG CUGAUGAGGCCGUUAGGCCCGAA AGGUGAAC | 10091 | |
| 4203 | AGGAAAAUC GCGUGCUG | 673 | CAGCACGC CUGAUGAGGCCGUUAGGCCCGAA AUUUUCCU | 10092 | |
| 4214 | GUGCUGCUC CCCGCCCC | 674 | GGGGCGGG CUGAUGAGGCCGUUAGGCCCGAA AGCAGCAC | 10093 | |
| 4229 | CCCAGACUA CAACUCGG | 675 | CCGAGUUG CUGAUGAGGCCGUUAGGCCCGAA AGUCUGGG | 10094 | |
| 4235 | CUACAACUC GGUGGUCC | 676 | GGACCACC CUGAUGAGGCCGUUAGGCCCGAA AGUUGUAG | 10095 | |
| 4242 | UCGGUGGUC CUGUACUC | 677 | GAGUACAG CUGAUGAGGCCGUUAGGCCCGAA ACCACCGA | 10096 | |
| 4247 | GGUCCUGUA CUCCACCC | 678 | GGGUGGAG CUGAUGAGGCCGUUAGGCCCGAA ACAGGACC | 10097 | |
| 4250 | CCUGUACUC CACCCCAC | 679 | GUGGGGUG CUGAUGAGGCCGUUAGGCCCGAA AGUACAGG | 10098 | |
| 4263 | CCACCCAUC UAGAGUUU | 680 | AAACUCUA CUGAUGAGGCCGUUAGGCCCGAA AUGGGUGG | 10099 | |
| 4265 | ACCCAUCUA GAGUUUGA | 681 | UCAAACUC CUGAUGAGGCCGUUAGGCCCGAA AGAUGGGU | 10100 | |
| 4270 | UCUAGAGUU UGACACGA | 682 | UCGUGUCA CUGAUGAGGCCGUUAGGCCCGAA ACUCUAGA | 10101 | |
| 4271 | CUAGAGUUU GACACGAA | 683 | UUCGUGUC CUGAUGAGGCCGUUAGGCCCGAA AACUCUAG | 10102 | |
| 4284 | CGAAGCCUU AUUUCUAG | 684 | CUAGAAAU CUGAUGAGGCCGUUAGGCCCGAA AGGCUUCG | 10103 | |
| 4285 | GAAGCCUUA UUUCUAGA | 685 | UCUAGAAA CUGAUGAGGCCGUUAGGCCCGAA AAGGCUUC | 10104 | |
| 4287 | AGCCUUAUU UCUAGAAG | 686 | CUUCUAGA CUGAUGAGGCCGUUAGGCCCGAA AUAAGGCU | 10105 | |
| 4288 | GCCUUAUUU CUAGAAGC | 687 | GCUUCUAG CUGAUGAGGCCGUUAGGCCCGAA AAUAAGGC | 10106 | |
| 4289 | CCUUAUUUC UAGAAGCA | 688 | UGCUUCUA CUGAUGAGGCCGUUAGGCCCGAA AAAUAAGG | 10107 | |
| 4291 | UUAUUUCUA GAAGCACA | 689 | UGUGCUUC CUGAUGAGGCCGUUAGGCCCGAA AGAAAUAA | 10108 | |
| 4305 | ACAUGUGUA UUUAUACC | 690 | GGUAUAAA CUGAUGAGGCCGUUAGGCCCGAA ACACAUGU | 10109 | |
| 4307 | AUGUGUAUU UAUACCCC | 691 | GGGGUAUA CUGAUGAGGCCGUUAGGCCCGAA AUACACAU | 10110 | |
| 4308 | UGUGUAUUU AUACCCCC | 692 | GGGGGUAU CUGAUGAGGCCGUUAGGCCCGAA AAUACACA | 10111 | |
| 4309 | GUGUAUUUA UACCCCCA | 693 | UGGGGGUA CUGAUGAGGCCGUUAGGCCCGAA AAAUACAC | 10112 | |
| 4311 | GUAUUUAUA CCCCCACG | 694 | CCUGGGGG CUGAUCAGGCCGUUAGGCCCGAA AUAAAUAC | 10113 | |
| 4325 | AGGAAACUA GCUUUUGC | 695 | CCAAAAGC CUGAUGAGGCCGUUAGGCCCGAA AGUUUCCU | 10114 | |
| 4329 | AACUAGCUU UUCCCAGU | 696 | ACUGGCAA CUGAUGAGGCCGUUAGGCCCGAA AGCUAGUU | 10115 | |
| 4330 | ACUAGCUUU UGCCAGUA | 697 | UACUGGCA CUGAUGAGGCCGUUAGGCCCGAA AAGCUAGU | 10116 | |
| 4331 | CUAGCUUUU GCCAGUAU | 698 | AUACUGGC CUGAUGAGGCCGUUAGGCCCGAA AAAGCUAG | 10117 | |
| 4338 | UUGCCAGUA UUAUGCAU | 699 | AUGCAUAA CUGAUGAGGCCGUUAGGCCCGAA ACUGGCAA | 10118 | |
| 4340 | GCCAGUAUU AUGCAUAU | 700 | AUAUGCAU CUGAUGAGGCCGUUAGGCCCGAA AUACUGGC | 10119 | |
| 4341 | CCAGUAUUA UGCAUAUA | 701 | UAUAUGCA CUGAUGAGGCCGUUACGCCCGAA AAUACUGG | 10120 | |
| 4347 | UUAUGCAUA UAUAAGUU | 702 | AACUUAUA CUGAUGAGGCCGUUAGGCCCGAA AUGCAUAA | 10121 | |
| 4349 | AUGCAUAUA UAAGUUUA | 703 | UAAACUUA CUGAUGAGGCCGUUAGGCCCGAA AUAUGCAU | 10122 | |
| 4351 | GCAUAUAUA AOUUUACA | 704 | UGUAAACU CUGAUGAGGCCGUUACGCCCGAA AUAUAUGC | 10123 | |
| 4355 | AUAUAAGUU UACACCUU | 705 | AAGGUGUA CUGAUCAGGCCGUUAGGCCCGAA ACUUAUAU | 10124 | |
| 4356 | UAUAAGUUU ACACCUUU | 706 | AAAGGUCU CUGAUCAGGCCGUUAGGCCCGAA AACUUAUA | 10125 | |
| 4357 | AUAAGUUUA CACCUUUA | 707 | UAAAGGUG CUGAUGAGGCCGUUAGGCCCGAA AAACUUAU | 10126 | |
| 4363 | UUACACCUU UAUCUUUC | 708 | GAAAGAUA CUGAUGAGGCCGUUAGGCCCGAA AGGUGUAA | 10127 | |
| 4364 | UACACCUUU AUCUUUCC | 709 | GGAAAGAU CUGAUGAGGCCGUUAGGCCCGAA AAGGUGUA | 10128 | |
| 4365 | ACACCUUUA UCUUUCCA | 710 | UGGAAAGA CUGAUGAGGCCGUUAGGCCCGAA AAAGGUGU | 10129 | |
| 4367 | ACCUUUAUC UUUCCAUC | 711 | CAUGGAAA CUGAUGAGGCCGUUAGGCCCGAA AUAAAGGU | 10130 | |
| 4369 | CUUUAUCUU UCCAUGGG | 712 | CCCAUGGA CUGAUGAGGCCGUUAGGCCCGAA AGAUAAAG | 10131 | |
| 4370 | UUUAUCUUU CCAUGGGA | 713 | UCCCAUGG CUGAUGAGGCCGUUAGGCCCGAA AAGAUAAA | 10132 | |
| 4371 | UUAUCUUUC CAUGOGAG | 714 | CUCCCAUG CUGAUGAGGCCGUUAGGCCCGAA AAAGAUAA | 10133 | |
| 4389 | CAGCUGCUU UUUGUGAU | 715 | AUCACAAA CUGAUGAGGCCGUUAGGCCCGAA AGCAGCUG | 10134 | |
| 4390 | AGCUGCUUU UUGUGAUU | 716 | AAUCACAA CUGAUGAGGCCGUUAGGCCCGAA AAGCAGCU | 10135 | |
| 4391 | GCUGCUUUU UGUGAUUU | 717 | AAAUCACA CUGAUGAGGCCGUUAGGCCCGAA AAAGCAGC | 10136 | |
| 4392 | CUGCUUUUU GUGAUUUU | 718 | AAAAUCAC CUGAUGAGGCCGUUAGGCCCGAA AAAAGCAG | 10137 | |
| 4398 | UUUGUGAUU UUUUUAAU | 719 | AUUAAAAA CUGAUGAGGCCGUUAGGCCCGAA AUCACAAA | 10138 | |
| 4399 | UUGUGAUUU UUUUAAUA | 720 | UAUUAAAA CUGAUGAGGCCGUUAGGCCCGAA AAUCACAA | 10139 | |
| 4400 | UGUGAUUUU UUUAAUAG | 721 | CUAUUAAA CUGAUGAGGCCGUUAGGCCCGAA AAAUCACA | 10140 | |
| 4401 | GUGAUUUUU UUAAUAGU | 722 | ACUAUUAA CUGAUGAGGCCGUUAGGCCCGAA AAAAUCAC | 10141 | |
| 4402 | UGAUUUUUU UAAUAGUG | 723 | CACUAUUA CUGAUGAGGCCGUUAGGCCCGAA AAAAAUCA | 10142 | |
| 4403 | GAUUUUUUU AAUAGUGC | 724 | GCACUAUU CUGAUGAGGCCGUUAGGCCCGAA AAAAAAUC | 10143 | |
| 4404 | AUUUUUUUA AUAGUGCU | 725 | AGCACUAU CUGAUGAGGCCGUUAGGCCCGAA AAAAAAAU | 10144 | |
| 4407 | UUUUUAAUA GUGCUUUU | 726 | AAAAGCAC CUGAUGAGGCCGUUAGGCCCGAA AUUAAAAA | 10145 | |
| 4413 | AUAGUGCUU UUUUUUUU | 727 | AAAAAAAA CUGAUGAGGCCGUUAGGCCCGAA AGCACUAU | 10146 | |
| 4414 | UAGUCCUUU UUUUUUUU | 728 | AAAAAAAA CUGAUGAGGCCGUUAGGCCCGAA AAGCACUA | 10147 | |
| 4415 | AGUGCUUUU UUUUUUUG | 729 | CAAAAAAA CUGAUGAGGCCGUUAGGCCCGAA AAAGCACU | 10148 | |
| 4416 | GUGCUUUUU UUUUUUGA | 730 | UCAAAAAA CUGAUGAGGCCGUUAGGCCCGAA AAAAGCAC | 10149 | |
| 4417 | UGCUUUUUU UUUUUGAC | 731 | GUCAAAAA CUGAUGAGGCCGUUAGGCCCGAA AAAAAGCA | 10150 | |
| 4418 | GCUUUUUUU UUUUGACU | 732 | AGUCAAAA CUGAUGAGGCCGUUAGGCCCGAA AAAAAAGC | 10151 | |
| 4419 | CUUUUUUUU UUUGACUA | 733 | UAGUCAAA CUGAUGAGGCCGUUAGGCCCGAA AAAAAAAG | 10152 | |
| 4420 | UUUUUUUUU UUGACUAA | 734 | UUAGUCAA CUGAUGAGGCCGUUAGGCCCGAA AAAAAAAA | 10153 | |
| 4421 | UUUUUUUUU UGACUAAC | 735 | GUUAGUCA CUGAUGAGGCCGUUAGGCCCGAA AAAAAAAA | 10154 | |
| 4422 | UUUUUUUUU GACUAACA | 736 | UGUUAGUC CUGAUGAGGCCGUUAGGCCCGAA AAAAkAAA | 10155 | |
| 4427 | UUUUGACUA ACAAGAAU | 737 | AUUCUUGU CUGAUGAGGCCGUUAGGCCCGAA AGUCAAAA | 10156 | |
| 4438 | AAGAAUGUA ACUCCAGA | 738 | UCUGGAGU CUGAUGAGGCCGUUAGGCCCGAA ACAUUCUU | 10157 | |
| 4442 | AUGUAACUC CAGAUAGA | 739 | UCUAUCUG CUGAUGAGGCCGUUAGGCCCGAA AGUUACAU | 10158 | |
| 4448 | CUCCAGAUA GAGAAAUA | 740 | UAUUUCUC CUGAUGAGGCCGUUAGGCCCGAA AUCUGGAG | 10159 | |
| 4456 | AGAGAAAUA GUGACAAG | 741 | CUUGUCAC CUGAUGAGGCCGUUACGCCCGAA AUUUCUCU | 10160 | |
| 4476 | AGAACACUA CUGCUAAA | 742 | UUUAGCAG CUGAUGAGGCCGUUAGGCCCGAA AGUGUUCU | 10161 | |
| 4482 | CUACUGCUA AAUCCUCA | 743 | UGAGGAUU CUGAUGAGGCCGUUAGGCCCGAA AGCAGUAG | 10162 | |
| 4486 | UGCUAAAUC CUCAUGUU | 744 | AACAUGAG CUGAUGAGGCCGUUAGGCCCGAA AUUUAGCA | 10163 | |
| 4489 | UAAAUCCUC AUGUUACU | 745 | AGUAACAU CUGAUGAGGCCGUUAGGCCCGAA AGGAUUUA | 10164 | |
| 4494 | CCUCAUGUU ACUCAGUG | 746 | CACUGAGU CUGAUGAGGCCGUUAGGCCCGAA ACAUGAGG | 10165 | |
| 4495 | CUCAUGUUA CUCAGUGU | 747 | ACACUGAG CUGAUGAGGCCGUUAGGCCCGAA AACAUGAG | 10166 | |
| 4498 | AUGUUACUC AGUGUUAG | 748 | CUAACACU CUGAUGAGGCCGUUAGGCCCGAA AGUAACAU | 10167 | |
| 4504 | CUCAGUGUG AGAGAAAU | 749 | AUUUCUCU CUGAUGAGGCCGUUAGGCCCGAA ACACUGAG | 10168 | |
| 4505 | UCAGUGUUA GAGAAAUC | 750 | GAUGUCUC CUGAUGAGGCCGUUAGGCCCGAA AACACUGA | 10169 | |
| 4513 | AGAGAAAUC CUUCCUAA | 751 | UUAGGAAG CUGAUGAGGCCGUUAGGCCCGAA AUUUCUCU | 10170 | |
| 4516 | GAAAUCCUU CCUAAACC | 752 | GGUUUAGG CUGAUGAGGCCGUUAGGCCCGAA AGGAUGUC | 10171 | |
| 4517 | AAAUCCUUC CUAAACCC | 753 | GGGUUUAG CUGAUGAGGCCGUUAGGCCCGAA AAGGAUUU | 10172 | |
| 4520 | UCCUUCCUA AACCCAAU | 754 | AUUGGGUU CUGAUGAGGCCGUUAGGCCCGAA AGGAAGGA | 10173 | |
| 4533 | CAAUGACUU CCCUGCUC | 755 | GAGCAGGG CUGAUGAGGCCGUUAGGCCCGAA AGUCAUUG | 10174 | |
| 4534 | AAUGACUUC CCUGCUCC | 756 | GGAGCAGG CUGAUGAGGCCGUUAGGCCCGAA AAGUCAUU | 10175 | |
| 4541 | UCCCUGCUC CAACCCCC | 757 | GGGGGUUG CUGAUGAGGCCGUUAGGCCCGAA AGCAGGGA | 10176 | |
| 4557 | CGCCACCUC AGGGCACG | 758 | CGUGCCCU CUGAUGAGGCCGUUAGGCCCGAA AGGUGGCG | 10177 | |
| 4576 | GGACCAGUU UGAUUGAG | 759 | CUCAAUCA CUGAUGAGGCCGUUAGGCCCGAA ACUGGUCC | 10178 | |
| 4577 | GACCAGUUU GAUUGAGG | 760 | CCUCAAUC CUGAUGAGGCCGUUAGGCCCGAA AACUGGUC | 10179 | |
| 4581 | AGUUUGAUU GAGGAGCU | 761 | AGCUCCUC CUGAUGAGGCCGUUAGGCCCGAA AUCAAACU | 10180 | |
| 4598 | GCACUGAUC ACCCAAUG | 762 | CAUUGGGU CUGAUGAGGCCGUUAGGCCCGAA AUCAGUGC | 10181 | |
| 4610 | CAAUGCAUC ACGUACCC | 763 | GGGUACGU CUGAUGAGGCCGUUAGGCCCGAA AUGCAUUG | 10182 | |
| 4615 | CAUCACGUA CCCCACUG | 764 | CAGUGGGG CUGAUGAGGCCGUUAGGCCCGAA ACGUGAUG | 10183 | |
| 4664 | AAGCCCGUU AGCCCCAG | 765 | CUGGGGCU CUGAUGAGGCCGUUAGGCCCGAA ACGGGCUU | 10184 | |
| 4665 | AGCCCGUUA GCCCCAGG | 766 | CCUGGGGC CUGAUGAGGCCGUUAGGCCCGAA AACGGGCU | 10185 | |
| 4678 | CAGOGGAUC ACUGGCUG | 767 | CAGCCAGU CUGAUGAGGCCGUUAGGCCCGAA AUCCCCUG | 10186 | |
| 4700 | AGCAACAUC UCGGGAGU | 768 | ACUCCCGA CUGAUGAGGCCGUUAGGCCCGAA AUGUUGCU | 10187 | |
| 4702 | CAACAUCUC GGGAGUCC | 769 | GGACUCCC CUGAUGAGGCCGUUAGGCCCGAA AGAUGUUG | 10188 | |
| 4709 | UCGGGAGUC CUCUAGCA | 770 | UCCUAGAG CUGAUGAGGCCGUUAGGCCCGAA ACUCCCGA | 10189 | |
| 4712 | GGAGUCCUC UAGCAGGC | 771 | GCCUGCUA CUGAUGAGGCCGUUAGGCCCGAA AGGACUCC | 10190 | |
| 4714 | AGUCCUCUA GCAGGCCU | 772 | AGGCCUGC CUGAUGAGGCCGUUAGGCCCGAA AGAGGACU | 10191 | |
| 4723 | GCAGGCCUA AGACAUGU | 773 | ACAUGUCU CUGAUGAGGCCGUUAGGCCCGAA AGGCCUGC | 10192 | |
| 4802 | GAAAGAAUU UGAGACGC | 774 | GCGUCUCA CUGAUGAGGCCGUUAGGCCCGAA AUUCUUUC | 10193 | |
| 4803 | AAAGAAUUU GAGACGCA | 775 | UGCGUCUC CUGAUGAGGCCGUUAGGCCCGAA AAUUCUUU | 10194 | |
| 4840 | ACGGGGCUC AGCAAUGC | 776 | GCAUUGCU CUGAUGAGGCCGUUAGGCCCGAA AGCCCCGU | 10195 | |
| 4852 | AAUGCCAUU UCAGUGGC | 777 | GCCACUGA CUGAUGAGGCCGUUAGGCCCGAA AUGGCAUU | 10196 | |
| 4853 | AUGCCAUUU CAGUGGCU | 778 | AGCCACUG CUGAUGAGGCCGUUAGGCCCGAA AAUGGCAU | 10197 | |
| 4854 | UGCCAUUUC AGUGGCUU | 779 | AAGCCACU CUGAUGAGGCCGUUAGGCCCGAA AAAUGGCA | 10198 | |
| 4862 | CAGUGGCUU CCCAGCUC | 780 | GAGCUGGG CUGAUGAGGCCGUUAGGCCCGAA AGCCACUG | 10199 | |
| 4863 | AGUGGCUUC CCAGCUCU | 781 | AGAGCUGG CUGAUGAGGCCGUUAGGCCCGAA AAGCCACU | 10200 | |
| 4870 | UCCCAGCUC UGACCCUU | 782 | AAGGGUCA CUGAUGAGGCCGUUAGGCCCGAA AGCUGGGA | 10201 | |
| 4878 | CUGACCCUU CUACAUUU | 783 | AAAUGUAG CUGAUGAGGCCGUUAGGCCCGAA AGGGUCAG | 10202 | |
| 4879 | UGACCCUUC UACAUUUG | 784 | CAAAUGUA CUGAUGAGGCCGUUAGGCCCGAA AAGGGUCA | 10203 | |
| 4881 | ACCCUUCUA CAUUUGAG | 785 | CUCAAAUG CUGAUGAGGCCGUUAGGCCCGAA AGAAGGGU | 10204 | |
| 4885 | UUCUACAUU UGAGGGCC | 786 | GGCCCUCA CUGAUGAGGCCGUUAGGCCCGAA AUGUAGAA | 10205 | |
| 4886 | UCUACAUUU GAGGGCCC | 787 | GGGCCCUC CUGAUGAGGCCGUUAGGCCCGAA AAUGUAGA | 10206 | |
| 4929 | GGGGACAUU UUCUGGAU | 788 | AUCCAGAA CUGAUGAGGCCGUUAGGCCCGAA AUGUCCCC | 10207 | |
| 4930 | GGGACAUUU UCUGGAUU | 789 | AAUCCAGA CUGAUGAGGCCGUUAGGCCCGAA AAUGUCCC | 10208 | |
| 4931 | GGACAUUUU CUGGAUUC | 790 | GAAUCCAG CUGAUGAGGCCGUUAGGCCCGAA AAAUGUCC | 10209 | |
| 4932 | GACAUUUUC UGGAUUCU | 791 | AGAAUCCA CUGAUGAGGCCGUUAGGCCCGAA AAAAUGUC | 10210 | |
| 4938 | UUCUGGAUU CUGGGAGG | 792 | CCUCCCAG CUGAUGAGGCCGUUAGGCCCGAA AUCCAGAA | 10211 | |
| 4939 | UCUGGAUUC UGGGAGGC | 793 | GCCUCCCA CUGAUGAGGCCGUUAGGCCCGAA AAUCCAGA | 10212 | |
| 4963 | GGACAAAUA UCUUUUUU | 794 | AAAAAAGA CUGAUGAGGCCGUUAGGCCCGAA AUUUGUCC | 10213 | |
| 4965 | ACAAAUAUC UUUUUUCC | 795 | CCAAAAAA CUGAUGAGGCCGUUAGGCCCGAA AUAUUUGU | 10214 | |
| 4967 | AAAUAUCUU UUUUGGAA | 796 | UUCCAAAA CUGAUGAGGCCGUUAGGCCCGAA AGAUAUUU | 10215 | |
| 4968 | AAUAUCUUU UUUGGAAC | 797 | GUUCCAAA CUGAUGAGGCCGUUAGGCCCGAA AACAUAUU | 10216 | |
| 4969 | AUAUCUUUU UUGGAACU | 798 | AGUUCCAA CUGAUGAGGCCGUUAGGCCCGAA AAAGAUAU | 10217 | |
| 4970 | UAUCUUUUU UGGAACUA | 799 | UAGUUCCA CUGAUGAGGCCGUUAGGCCCGAA AAAACAUA | 10218 | |
| 4971 | AUCUUUUUU GGAACUAA | 800 | UUAGUUCC CUGAUGAGGCCGUUAGGCCCGAA AAAAAGAU | 10219 | |
| 4978 | UUGGAACUA AAGCAAAU | 801 | AUUUGCUU CUGAUGAGGCCGUUAGGCCCGAA AGUUCCAA | 10220 | |
| 4987 | AAGCAAAUU UUAGACCU | 802 | AGGUCUAA CUGAUGAGGCCGUUAGGCCCGAA AUUUGCUU | 10221 | |
| 4988 | AGCAAAUUU UAGACCUU | 803 | AAGGUCUA CUGAUGAGCCCGUUAGGCCCGAA AAUUUGCU | 10222 | |
| 4989 | GCAAAUUUU AGACCUUU | 804 | APAGGUCU CUGAUGAGGCCGUUAGGCCCGAA AAAUUUGC | 10223 | |
| 4990 | CAAAUUUUA GACCUUUA | 805 | UAAAGGUC CUGAUGAGGCCGUUAGGCCCGAA AAAAUUUG | 10224 | |
| 4996 | UUAGACCUU UACCUAUG | 806 | CAUAGGUA CUGAUGAGGCCGUUAGGCCCGAA AGGUCUAA | 10225 | |
| 4997 | UAGACCUUU ACCUAUGG | 807 | CCAUAGGU CUCAUGAGGCCGUUAGGCCCGAA AAGGUCUA | 10226 | |
| 4998 | AGACCUUUA CCUAUGGA | 808 | UCCAUAGG CUGAUGAGGCCGUUAGGCCCGAA AAAGGUCU | 10227 | |
| 5002 | CUUUACCUA UGGAAGUG | 809 | CACUUCCA CUGAUGAGGCCGUUAGGCCCGAA AGGUAAAG | 10228 | |
| 5013 | GAAGUGGUU CUAUGUCC | 810 | GGACAUAG CUGAUGAGGCCGUUAGGCCCGAA ACCACUUC | 10229 | |
| 5014 | AAGUGGUUC UAUGUCCA | 811 | UGGACAUA CUGAUGAGGCCGUUACGCCCGAA AACCACUU | 10230 | |
| 5016 | GUGGUUCUA UGUCCAUU | 812 | AAUGGACA CUGAUGAGGCCGUUAGGCCCGAA AGAACCAC | 10231 | |
| 5020 | UUCUAUGUC CAUUCUCA | 813 | UGAGAAUG CUGAUGAGGCCGUUAGGCCCGAA ACAUAGAA | 10232 | |
| 5024 | AUGUCCAUU CUCAUUCG | 814 | CGAAUGAG CUGAUGAGGCCGUUAGGCCCGAA AUGCACAU | 10233 | |
| 5025 | UGUCCAUUC UCAUUCGU | 815 | ACGAAUGA CUGAUCAGGCCGUUAGGCCCGAA AAUGGACA | 10234 | |
| 5027 | UCCAUUCUC AUUCGUGG | 816 | CCACGAAU CUGAUGAGGCCGUUAGGCCCGAA AGAAUGGA | 10235 | |
| 5030 | AUUCUCAUU CGUGGCAU | 817 | AUGCCACG CUGAUGAGGCCGUUAGGCCCGAA AUGAGAAU | 10236 | |
| 5031 | UUCUCAUUC GUGGCAUG | 818 | CAUGCCAC CUGAUGAGGCCGUUAGGCCCGAA AAUGAGAA | 10237 | |
| 5041 | UGGCAUGUU UUGAUUUG | 819 | CAAAUCAA CUGAUGAGGCCGUUAGGCCCGAA ACAUGCCA | 10238 | |
| 5042 | GGCAUGUUU UGAUUUGU | 820 | ACAAAUCA CUGAUCAGGCCGUUAGGCCCGAA AACAUCCC | 10239 | |
| 5043 | CCAUGUUUU GAUUUGUA | 821 | UACAAAUC CUGAUGAGGCCGUUAGCCCCGAA AAACAUGC | 10240 | |
| 5047 | GUUUUGAUU UGUAGCAC | 822 | GUGCUACA CUGAUGAGGCCGUUAGGCCCGAA AUCAAAAC | 10241 | |
| 5048 | UUUUGAUUU GUAGCACU | 823 | AGUGCUAC CUGAUGAGGCCGUUAGGCCCGAA AAUCAAAA | 10242 | |
| 5051 | UGAUUUGUA GCACUGAC | 824 | CUCAGUGC CUGAUGAGGCCGUUAGGCCCGAA ACAAAUCA | 10243 | |
| 5069 | GUGGCACUC AACUCUGA | 825 | UCAGAGUU CUGAUGAGGCCGUUAGGCCCGAA AGUGCCAC | 10244 | |
| 5074 | ACUCAACUC UGAGCCCA | 826 | UGGGCUCA CUGAUGAGGCCGUUAGGCCCGAA AGUUCAGU | 10245 | |
| 5084 | GAGCCCAUA CUUUUCGC | 827 | GCCAAAAG CUGAUGAGGCCGUUAGGCCCGAA AUGGGCUC | 10246 | |
| 5087 | CCCAUACUU UUGGCUCC | 828 | GGACCCAA CUGAUGAGGCCGUUAGGCCCGAA ACUAUGGG | 10247 | |
| 5088 | CCAUACUUU UGGCUCCU | 829 | AGGAGCCA CUGAUGAGGCCGUUAGGCCCGAA AAGUAUGG | 10248 | |
| 5089 | CAUACUUUU GGCUCCUC | 830 | GAGGAGCC CUGAUGAGGCCGUUAGGCCCGAA AAAGUAUG | 10249 | |
| 5094 | UUUUGGCUC CUCUAGUA | 831 | UACUAGAG CUGAUGAGGCCGUUAGGCCCGAA AGCCAAAA | 10250 | |
| 5097 | UGGCUCCUC UAGUAAGA | 832 | UCUUACUA CUGAUGAGGCCGUUAGGCCCGAA AGGAGCCA | 10251 | |
| 5099 | GCUCCUCUA GUAAGAUG | 833 | CAUCUUAC CUGAUGAGGCCGUUAGGCCCGAA AGAGGAGC | 10252 | |
| 5102 | CCUCUAGUA AGAUGCAC | 834 | GUGCAUCU CUGAUGAGGCCGUUAGGCCCGAA ACUAGAGG | 10253 | |
| 5119 | UGAAAACUU AGCCAGAG | 835 | CUCUGGCU CUGAUGAGGCCGUUAGGCCCGAA ACUUUUCA | 10254 | |
| 5120 | GAAAACUUA CCCAGAGU | 836 | ACUCUGGC CUGAUGAGGCCGUUAGGCCCGAA AAGUUUUC | 10255 | |
| 5129 | GCCAGAGUU AGGUUGUC | 837 | CACAACCU CUGAUGAGGCCGUUAGGCCCGAA ACUCUGGC | 10256 | |
| 5130 | CCACAGUUA GGUUGUCU | 838 | AGACAACC CUGAUGAGGCCGUUACGCCGGAA AACUCUGG | 10257 | |
| 5134 | AGUUAGGUU GUCUCCAG | 839 | CUGGAGAC CUGAUCAGGCCGUUAGGCCGGAA ACCUAACU | 10258 | |
| 5137 | UAGGUUGUC UCCAGGCC | 840 | GGCCUGGA CUGAUGAGGCCGUUAGGCCCGAA ACAACCUA | 10259 | |
| 5139 | GGUUGUCUC CAGGCCAU | 841 | AUGGCCUG CUGAUGAGGCCGUUAGGCCCGAA AGACAACC | 10260 | |
| 5156 | GAUGGCCUU ACACUGAA | 842 | UUCAGUGU CUGAUGAGGCCGUUAGGCCCGAA AGGCCAUC | 10261 | |
| 5157 | AUGGCCUUA CACUGAAA | 843 | UUUCAGUG CUCAUGAGGCCGUUAGGCCCGAA AAGGCCAU | 10262 | |
| 5170 | GAAAAUGUC ACAUUCUA | 844 | UAGAAUGU CUGAUGAGGCCGUUAGGCCCGAA ACAUUUUC | 10263 | |
| 5175 | UGUCACAUU CUAUUUUG | 845 | CAAAAUAG CUGAUGAGGCCGUUAGGCCCGAA AUGUGACA | 10264 | |
| 5176 | GUCACAUUC UAUUUUGG | 846 | CCAAAAUA CUGAUGAGGCCGUUAGGCCCGAA AAUGUGAC | 10265 | |
| 5178 | CACAUUCUA UUUUGGGU | 847 | ACCCAAAA CUGAUGAGGCCGUUAGGCCCGAA AGAAUGUG | 10266 | |
| 5180 | CAUUCUAUU UUGGGUAU | 848 | AUACCCAA CUGAUGAGGCCGUUAGGCCCGAA AUAGAAUG | 10267 | |
| 5181 | AUUCUAUUU UGGGUAUU | 849 | AAUACCCA CUGAUGAGGCCGUUAGGCCCGAA AAUAGAAU | 10268 | |
| 5182 | UUCUAUUUU GGGUAUUA | 850 | UAAUACCC CUGAUGAGGCCGUUAGGCCCGAA AAAUAGAA | 10269 | |
| 5187 | UUUUGGGUA UUAAUAUA | 851 | UAUAUUAA CUGAUGAGGCCGUUAGGCCCGAA ACCCAAAU | 10270 | |
| 5189 | UUGGGUAUU AAUAUAUA | 852 | UAUAUAUU CUGAUGAGGCCGUUAGGCCCGAA AUACCCAA | 10271 | |
| 5190 | UCGGUAUUA AUAUAUAG | 853 | CUAUAUAU CUGAUGAGGCCGUUAGGCCCGAA AAUACCCA | 10272 | |
| 5193 | GUAUUAAUA UAUAGUCC | 854 | GGACUAUA CUGAUGAGGCCGUUAGGCCCGAA AUUAAUAC | 10273 | |
| 5195 | AUUAAUAUA UAGUCCAG | 855 | CUGGACUA CUGAUGAGGCCGUUAGGCCCGAA AUAUUAAU | 10274 | |
| 5197 | UAAUAUAUA GUCCAGAC | 856 | GUCUGGAC CUGAUGAGGCCGUUAGGCCCGAA AUAUAUUA | 10275 | |
| 5200 | UAUAUAGUC CAGACACU | 857 | AGUGUCUG CUGAUGAGGCCGUUAGGCCCGAA ACUAHAHA | 10276 | |
| 5209 | CAGACACUU AACUCAAU | 858 | AUUGAGUU CUGAUGAGGCCGUUAGGCCCGAA AGUGUCUG | 10277 | |
| 5210 | AGACACUUA ACUCAAUU | 859 | AAUUGAGU CUGAUGAGGCCGUUAGGCCCGAA AAGUGUCU | 10278 | |
| 5214 | ACUUAACUC AAUUUCUU | 860 | AAGAAAUU CUGAUGAGGCCGUUAGGCCCGAA AGUUAAGU | 10279 | |
| 5218 | AACUCAAUU UCUUGGUA | 861 | UACCAAGA CUGAUGAGGCCGUUAGGCCCGAA AUUGAGUU | 10280 | |
| 5219 | ACUCAAUUU CUUGGUAU | 862 | AUACCAAG CUGAUGAGGCCGUUAGGCCCGAA AAUUGAGU | 10281 | |
| 5220 | CUCAAUUUC UUGGUAUU | 863 | AAUACCAA CUGAUGAGGCCGUUAGGCCCGAA AAAUUGAG | 10282 | |
| 5222 | CAAUUUCUU GGUAUUAU | 864 | AUAAUACC CUGAUGAGGCCGUUAGGCCCGAA AGAAAUUG | 10283 | |
| 5226 | UUCUUGGUA UUAUUCUG | 865 | CAGAAUAA CUGAUGAGGCCGUUAGGCCCGAA ACCAAGAA | 10284 | |
| 5228 | CUUGGUAUU AUUCUGUU | 866 | AACAGAAU CUGAUGAGGCCGUUAGGCCCGAA AUACCAAG | 10285 | |
| 5229 | UUGGUAUUA UUCUGUUU | 867 | AAACAGAA CUGAUGAGGCCGUUAGGCCCGAA AAUACCAA | 10286 | |
| 5231 | GGUAUUAUU CUGUUUUG | 868 | CAAAACAG CUGAUGAGGCCGUUAGGCCCGAA AUAAUACC | 10287 | |
| 5232 | GUAUUAUUC UGUUUUGC | 869 | GCAAAACA CUGAUGAGGCCGUUAGGCCCGAA AAUAAUAC | 10288 | |
| 5236 | UAUUCUGUU UUGCACAG | 870 | CUGUGCAA CUGAUGAGGCCGUUAGGCCCGAA ACAGAAUA | 10289 | |
| 5237 | AUUCUGUUU UGCACAGU | 871 | ACUGUGCA CUGAUGAGGCCGUUAGGCCCGAA AACAGAAU | 10290 | |
| 5238 | UUCUGUUUU GCACAGUU | 872 | AACUGUGC CUGAUGAGGCCGUUAGGCCCGAA AAACAGAA | 10291 | |
| 5246 | UGCACAGUU AGUUGUGA | 873 | UCACAACU CUGAUGAGGCCGUUAGGCCCGAA ACUGUGCA | 10292 | |
| 5247 | GCACAGUUA GUUGUGAA | 874 | UUCACAAC CUGAUGAGGCCGUUAGGCCCGAA AACUGUGC | 10293 | |
| 5250 | CAGUUAGUU GUGAAAGA | 875 | UCUGUCAC CUGAUGAGGCCGUUAGGCCCGAA ACUAACUG | 10294 | |
| 5284 | AAUGCAGUC CUGAGGAG | 876 | CUCCUCAG CUGAUGAGGCCGUUAGGCCCGAA ACUGCAUU | 10295 | |
| 5296 | AGGAGAGUU UUCUCCAU | 877 | AUGGAGAA CUGAUGAGGCCGUUAGGCCCGAA ACUCUCCU | 10296 | |
| 5297 | GGAGAGUUU UCUCCAUA | 878 | UAUGGAGA CUGAUGAGGCCGUUAGGCCCGAA AACUCUCC | 10297 | |
| 5298 | GAGAGUUUU CUCCAUAU | 879 | AUAUGGAG CUGAUGAGGCCGUUAGGCCCGAA AAACUCUC | 10298 | |
| 5299 | AGAGUUUUC UCCAUAUC | 880 | GAUAUGGA CUGAUGAGGCCGUUAGGCCCGAA AAAACUCU | 10299 | |
| 5301 | AGUUUUCUC CAUAUCAA | 881 | UUGAUAUG CUGAUGAGGCCGUUAGGCCCGAA AGAAAACU | 10300 | |
| 5305 | UUCUCCAUA UCAAAACG | 882 | CGUUUUGA CUGAUGAGGCCGUUAGGCCCGAA AUGGAGAA | 10301 | |
| 5307 | CUCCAUAUC AAAACGAG | 883 | CUCGUUUU CUGAUGAGGCCGUUAGGCCCGAA AUAUGGAG | 10302 | |
| 5336 | AAAAAGGUC AAUAAGGU | 884 | ACCUUAUU CUGAUGAGGCCGUUAGGCCCGAA ACCUUUUU | 10303 | |
| 5340 | AGGUCAAUA AGGUCAAG | 885 | CUUGACCU CUGAUGAGGCCGUUAGGCCCGAA AUUGACCU | 10304 | |
| 5345 | AAUAAGGUC AAGGGAAG | 886 | CUUCCCUU CUGAUGAGGCCGUUAGGCCCGAA ACCUUAUU | 10305 | |
| 5361 | GACCCCGUC UCUAUACC | 887 | GGUAUAGA CUGAUGAGGCCGUUAGGCCCGAA ACGGGGUC | 10306 | |
| 5363 | CCCCGUCUC UAUACCAA | 888 | UUGGUAUA CUGAUGAGGCCGUUAGGCCCGAA AGACGGGG | 10307 | |
| 5365 | CCGUCUCUA UACCAACC | 889 | GGUUGGUA CUGAUGAGGCCGUUAGGCCCGAA AGAGACGG | 10308 | |
| 5367 | GUCUCUAUA CCAACCAA | 890 | UUGGUUGG CUGAUGAGGCCGUUAGGCCCGAA AUAGAGAC | 10309 | |
| 5382 | AAACCAAUU CACCAACA | 891 | UGUUGGUG CUGAUGAGGCCGUUAGGCCCGAA AUUGGUUU | 10310 | |
| 5383 | AACCAAUUC ACCAACAC | 892 | GUGUUGGU CUGAUGAGGCCGUUAGGCCCGAA AAUUGGUU | 10311 | |
| 5395 | AACACAGUU GGGACCCA | 893 | UGGGUCCC CUGAUGAGGCCGUUAGGCCCGAA ACUGUGUU | 10312 | |
| 5417 | CAGGAAGUC AGUCACGU | 894 | ACGUGACU CUGAUGAGGCCGUUAGGCCCGAA ACUUCCUG | 10313 | |
| 5421 | AAGUCAGUC ACGUUUCC | 895 | GGAAACGU CUGAUGAGGCCGUUAGGCCCGAA ACUGACUU | 10314 | |
| 5426 | AGUCACGUU UCCUUUUC | 896 | GAAPAGGA CUGAUGAGGCCGUUAGGCCCGAA ACGUGACU | 10315 | |
| 5427 | GUCACGUUU CCUUUUCA | 897 | UGAAAAGG CUGAUGAGGCCGUUAGGCCCGAA AACGUGAC | 10316 | |
| 5428 | UCACGUUUC CUUUUCAU | 898 | AUGAAAAG CUGAUGAGGCCGUUAGGCCCGAA AAACGUGA | 10317 | |
| 5431 | CGUUUCCUU UUCAUUUA | 899 | UAAAUGAA CUGAUGAGGCCGUUAGGCCCGAA AGGAAACG | 10318 | |
| 5432 | GUUUCCUUU UCAUUUAA | 900 | UUAAAUGA CUGAUGAGGCCGUUAGGCCCGAA AAGGAAAC | 10319 | |
| 5433 | UUUCCUUUU CAUUUAAU | 901 | AUUAPAUG CUGAUGAGGCCGUUAGGCCCGAA AAAGGAAA | 10320 | |
| 5434 | UUCCUUUUC AUUUAAUG | 902 | CAUUAAAU CUGAUGAGGCCGUUAGGCCCGAA AAAAGGAA | 10321 | |
| 5437 | CUUUUCAUU UAAUGGGG | 903 | CCCCAUUA CUGAUGAGGCCGUUAGGCCCGAA AUGAAAAG | 10322 | |
| 5438 | UUUUCAUUU AAUGGGGA | 904 | UCCCCAUU CUGAUGAGGCCGUUAGGCCCGAA ZAAUGAAAA | 10323 | |
| 5439 | UUUCAUUUA AUGGGGAU | 905 | AUCCCCAU CUGAUGAGGCCGUUAGGCCCGAA AAAUGAAA | 10324 | |
| 5448 | AUGGGGAUU CCACUAUC | 906 | CAUAGUGG CUGAUGAGGCCGUUAGGCCCGAA AUCCCCAU | 10325 | |
| 5449 | UGGGCAUUC CACUAUCU | 907 | AGAUAGUG CUGAUGAGGCCGUUAGGCCCGAA AAUCCCCA | 10326 | |
| 5454 | AUUCCACUA UCUCACAC | 908 | GUGUGAGA CUGAUGAGGCCGUUAGGCCCGAA AGUGGAAU | 10327 | |
| 5456 | UCCACUAUC UCACACUA | 909 | UAGUGUGA CUGAUGAGGCCGUUAGGCCCGAA AUAGUGGA | 10328 | |
| 5458 | CACUAUCUC ACACUAAU | 910 | AUUAGUGU CUGAUGAGGCCGUUAGGCCCGAA AGAUAGUG | 10329 | |
| 5464 | CUCACACUA AUCUGAAA | 911 | UUUCAGAU CUGAUGAGGCCGUUAGGCCCGAA AGUGUGAG | 10330 | |
| 5467 | ACACUAAUC UGAAAGGA | 912 | UCCUUUCA CUGAUGAGGCCGUUAGGCCCGAA AUUAGUGU | 10331 | |
| 5489 | AAGAGCAUU AGCUGGCG | 913 | CGCCAGCU CUGAUGAGGCCGUUAGGCCCGAA AUGCUCUU | 10332 | |
| 5490 | AGAGCAUUA GCUGGCGC | 914 | GCGCCAGC CUGAUGAGGCCGUUAGGCCCGAA AAUGCUCU | 10333 | |
| 5501 | UGGCGCAUA UUAAGCAC | 915 | GUGCUUAA CUGAUGAGGCCGUUAGGCCCGAA AUCCUCCA | 10334 | |
| 5503 | GCGCAUAUU AAGCACUU | 916 | AAGUGCUU CUGAUGAGGCCGUUAGGCCCGAA AUAUGCGC | 10335 | |
| 5504 | CGCAUAUUA AGCACUUU | 917 | AAAGUGCU CUGAUGAGGCCGUUAGGCCCGAA AAUAUGCG | 10336 | |
| 5511 | UAAGCACUU UAAGCUCC | 918 | GUACCUGA CUGAUGAGGCCGUUAGGCCCGAA AGUGCUUA | 10337 | |
| 5512 | AAGCACUUU AAGCUCCU | 919 | AGGAGCUU CUGAUGAGGCCGUUAGGCCCGAA AAGUGCUU | 10338 | |
| 5513 | AGCACUUUA AGCUCCUU | 920 | AAGGAGCU CUGAUGAGGCCGUUAGGCCCGAA AAAGUGCU | 10339 | |
| 5518 | UUUAAGCUC CUUGAGUA | 921 | UACUCAAG CUGAUGAGGCCGUUAGGCCCGAA AGCUUAAA | 10340 | |
| 5521 | AAGCUCCUU GAGUAAAA | 922 | UUUUACUC CUGAUGAGGCCGUUAGGCCCGAA AGGAGCUU | 10341 | |
| 5526 | CCUUGAGUA AAAAGGUG | 923 | CACCUUUU CUGAUGAGGCCGUUAGGCCCGAA ACUCAAGG | 10342 | |
| 5537 | AAGGUGGUA UGUAAUUU | 924 | AAAUUACA CUGAUGAGGCCGUUAGGCCCGAA ACCACCUU | 10343 | |
| 5541 | UGGUAUGUA AUUUAUGC | 925 | GCAUAAAU CUGAUGAGGCCGUUAGGCCCGAA ACAUACCA | 10344 | |
| 5544 | UAUGUAAUU UAUGCAAG | 926 | CUUGCAUA CUGAUGAGGCCGUUAGGCCCGAA AUUACAUA | 10345 | |
| 5545 | AUGUAAUUU AUGCAAGG | 927 | CCUUGCAU CUGAUGAGGCCGUUAGGCCCGAA AAUUACAU | 10346 | |
| 5546 | UGUAAUUUA UGCAAGGU | 928 | ACCUUGCA CUGAUGAGGCCGUUAGGCCCGAA AAAUUACA | 10347 | |
| 5555 | UGCAAGGUA UUUCUCCA | 929 | UGGAGAAA CUGAUGAGGCCGUUAGGCCCGAA ACCUUGCA | 10348 | |
| 5557 | CAAGGUAUU UCUCCAGU | 930 | ACUGGAGA CUGAUGAGGCCGUUAGGCCCGAA AUACCUUG | 10349 | |
| 5558 | AAGGUAUUU CUCCAGUU | 931 | AACUGGAG CUGAUGAGGCCGUUAGGCCCGAA AAUACCUU | 10350 | |
| 5559 | AGGUAUUUC UCCAGUUG | 932 | CAACUGGA CUGAUGAGGCCGUUAGGCCCGAA AAAUACCU | 10351 | |
| 5561 | GUAUUUCUC CAGUUGGG | 933 | CCCAACUG CUGAUGAGGCCGUUAGGCCCGAA AGAAAUAC | 10352 | |
| 5566 | UCUCCAGUU GGGACUCA | 934 | UGAGUCCC CUGAUGAGGCCGUUAGGCCCGAA ACUGGAGA | 10353 | |
| 5573 | UUGGGACUC AGGAUAUU | 935 | AAUAUCCU CUGAUGAGGCCGUUAGGCCCGAA AGUCCCAA | 10354 | |
| 5579 | CUCAGGAUA UUAGUUAA | 936 | UUAACUAA CUGAUGAGGCCGUUAGGCCCGAA AUCCUGAG | 10355 | |
| 5581 | CAGGAUAUU AGUUAAUG | 937 | CAUUAACU CUGAUGAGGCCGUUAGGCCCGAA AUAUCCUG | 10356 | |
| 5582 | AGGAUAUUA GUUAAUGA | 938 | UCAUUAAC CUGAUGAGGCCGUUAGGCCCGAA AAUAUCCU | 10357 | |
| 5585 | AUAUUAGUU AAUGAGCC | 939 | GGCUCAUU CUGAUGAGGCCGUUAGGCCCGAA ACUAAUAU | 10358 | |
| 5586 | UAUUAGUUA AUGAGCCA | 940 | UGGCUCAU CUGAUGAGGCCGUUAGGCCCGAA AACUAAUA | 10359 | |
| 5596 | UGAGCCAUC ACUAGAAG | 941 | CUUCUAGU CUGAUGAGGCCGUUAGGCCCGAA AUGUCUCA | 10360 | |
| 5600 | CCAUCACUA GAAGAAAA | 942 | UUUUCUUC CUGAUGAGGCCGUUAGGCCCGAA AGUGAUGG | 10361 | |
| 5615 | AAGCCCAUU UUCAACUG | 943 | CAGUUGAA CUGAUGAGGCCGUUAGGCCCGAA AUGGGCUU | 10362 | |
| 5616 | AGCCCAUUU UCAACUGC | 944 | GCAGUUGA CUGAUGAGGCCGUUAGGCCCGAA AAUGGGCU | 10363 | |
| 5617 | GCCCAUUUU CAACUGCU | 945 | AGCAGUUG CUGAUGAGGCCGUUAGGCCCGAA AAAUGGGC | 10364 | |
| 5618 | CCCAUUUUC AACUGCUU | 946 | AAGCAGUU CUGAUGAGGCCGUUAGGCCCGAA AAAAUGGG | 10365 | |
| 5626 | CAACUGCUU UGAAACUU | 947 | AAGUUUCA CUGAUGAGGCCGUUAGGCCCGAA AGCAGUUG | 10366 | |
| 5627 | AACUGCUUU GAAACUUG | 948 | CAACUUUC CUGAUGAGGCCGUUAGGCCCGAA AAGCAGUU | 10367 | |
| 5634 | UUGAAACUU GCCUGGGG | 949 | CCCCAGGC CUCAUGAGGCCGUUAGGCCCGAA AGUUUCAA | 10368 | |
| 5644 | CCUGGGGUC UGAGCAUG | 950 | CAUGCUCA CUGAUGAGGCCGUUAGGCCCGAA ACCCCAGG | 10369 | |
| 5661 | AUGGGAAUA GGGAGACA | 951 | UCUCUCCC CUGAUGAGGCCGUUAGGCCCGAA AUUCCCAU | 10370 | |
| 5674 | GACAGGGUA GGAAAGGG | 952 | CCCUUUCC CUGAUGAGGCCGUUAGGCCCGAA ACCCUGUC | 10371 | |
| 5688 | GGGCGCCUA CUCUUCAG | 953 | CUGAAGAG CUGAUGAGGCCGUUAGGCCCGAA AGGCGCCC | 10372 | |
| 5691 | CGCCUACUC UUCAGGUU | 954 | ACCCUGAA CUGAUGAGGCCGUUAGGCCCGAA AGUAGGCG | 10373 | |
| 5693 | CCUACUCUU CAGGGUCU | 955 | AGACCCUG CUGAUGAGGCCGUUAGGCCCGAA AGAGUAGG | 10374 | |
| 5694 | CUACUCUUC AGGGUCUA | 956 | UAGACCCU CUGAUGAGGCCGUUAGGCCCGAA AAGAGUAG | 10375 | |
| 5700 | UUCAGGGUC UAAAGAUC | 957 | GAUCUUHA CUGAUGAGGCCGUUAGGCCCGAA ACCCUGAA | 10376 | |
| 5702 | CAGGGUCUA AAGAUCAA | 958 | UUGAUCUU CUGAUGAGGCCGUUAGGCCCGAA AGACCCUG | 10377 | |
| 5708 | CUAAAGAUC AAGUGGGC | 959 | GCCCACUU CUGAUGAGGCCGUUAGGCCCGAA AUCUUUAG | 10378 | |
| 5719 | GUGGGCCUU GGAUCGCU | 960 | AGCGAUCC CUGAUGAGGCCGUUAGGCCCGAA AGGCCCAC | 10379 | |
| 5724 | CCUUGGAUC GCUAAGCU | 961 | AGCUUAGC CUGAUGAGGCCGUUAGGCCCGAA AUCCAAGG | 10380 | |
| 5728 | GGAUCGCUA AGCUGGCU | 962 | AGCCAGCU CUGAUGAGGCCGUUAGGCCCGAA AGCGAUCC | 10381 | |
| 5737 | AGCUGGCUC UGUUUGAU | 963 | AUCAAACA CUGAUGAGGCCGUUAGGCCCGAA AGCCAGCU | 10382 | |
| 5741 | GOCUCUGUU UGAUGCUA | 964 | UAGCAUCA CUGAUGAGGCCGUUAGGCCCGAA ACAGAGCC | 10383 | |
| 5742 | GCUCUGUUU GAUGCUAU | 965 | AUAGCAUC CUGAUGAGGCCGUUAGGCCCGAA AACAGAGC | 10384 | |
| 5749 | UUGAUGCUA UUCAUCCA | 966 | UGCAUAAA CUGAUGAGGCCGUUAGGCCCGAA AGCAUCAA | 10385 | |
| 5751 | GAUGCUAUU UAUGCAAG | 967 | CUUGCAUA CUGAUGAGGCCGUUAGGCCCGAA AUAGCAUC | 10386 | |
| 5752 | AUGCUAUUU AUGCAAGU | 968 | ACUUCCAU CUGAUGAGGCCGUUAGGCCCGAA AAUAGCAU | 10387 | |
| 5753 | UGCUAUUUA UGCAAGUU | 969 | AACUUGCA CUGAUGAGGCCGUUAGGCCCGAA AAAUAGCA | 10388 | |
| 5761 | AUGCAAGUU AGGGUCUA | 970 | UAGACCCU CUGAUGAGGCCGUUAGGCCCGAA ACUUGCAU | 10389 | |
| 5762 | UGCAAGUUA GGGUCUAU | 971 | AUAGACCC CUGAUGAGGCCGUUAGGCCCGAA AACUUGCA | 10390 | |
| 5767 | GUUAGGGUC UAUGUAUU | 972 | AAUACAUA CUGAUGAGGCCGUUAGGCCCGAA ACCCUAAC | 10391 | |
| 5769 | UAGGGUCUA UGUAUUUA | 973 | UAAAUACA CUGAUGAGGCCGUUAGGCCCGAA AGACCCUA | 10392 | |
| 5773 | GUCUAUGUA UUUAGGAU | 974 | AUCCUAAA CUGAUGAGGCCGUUAGGCCCGAA ACAUAGAC | 10393 | |
| 5775 | CUAUGUAUU UAGGAUGC | 975 | GCAUCCUA CUGAUGAGGCCGUUAGGCCCGAA AUACAUAG | 10394 | |
| 5776 | UAUGUAUUU AGGAUGCG | 976 | CGCAUCCU CUGAUGAGGCCGUUAGGCCCGAA AAUACAUA | 10395 | |
| 5777 | AUGUAUUUA GGAUGCGC | 977 | GCGCAUCC CUGAUGAGGCCGUUAGGCCCGAA AAAUACAU | 10396 | |
| 5788 | AUGCGCCUA CUCUUCAG | 978 | CUGAAGAG CUGAUGAGGCCGUUAGGCCCGAA AGGCGCAU | 10397 | |
| 5791 | CGCCUACUC UUCAGGGU | 954 | ACCCUGAA CUGAUGAGGCCGUUAGGCCCGAA AGUAGGCG | 10373 | |
| 5793 | CCUACUCUU CAGGGUCU | 955 | AGACCCUG CUGAUGAGGCCGUUAGGCCCGAA AGAGUAGG | 10374 | |
| 5794 | CUACUCUUC AGGGUCUA | 956 | UAGACCCU CUGAUGAGGCCGUUAGGCCCGAA AAGAGUAG | 10375 | |
| 5800 | UUCAGGGUC UAAAGAUC | 957 | GAUCUUUA CUGAUGAGGCCGUUAGGCCCGAA ACCCUGUA | 10376 | |
| 5802 | CAGGGUCUA AAGAUCAA | 958 | UUGAUCUU CUGAUGAGGCCGUUAGGCCCGAA AGACCCUG | 10377 | |
| 5808 | CUAAAGAUC AAGUGGGC | 959 | GCCCACUU CUGAUGAGGCCGUUAGGCCCGAA AUCUUUAG | 10378 | |
| 5819 | GUGGGCCUU GGAUCGCU | 960 | AGCGAUCC CUGAUGAGGCCGUUAGGCCCGAA AGGCCCAC | 10379 | |
| 5824 | CCUUGGAUC GCUAAGCU | 961 | AGCUUAGC CUGAUGAGGCCGUUAGGCCCGAA AUCCAAGG | 10380 | |
| 5828 | GGAUCGCUA AGCUGGCU | 962 | AGCCAGCU CUGAUGAGGCCGUUAGGCCCGAA AGCGAUCC | 10381 | |
| 5837 | AGCUGGCUC UGUUUGAU | 963 | AUCAAACA CUGAUGAGGCCGUUAGGCCCGAA AGCCAGCU | 10382 | |
| 5841 | GGCUCUGUU UGAUGCUA | 964 | UAGCAUCA CUGAUGAGGCCGUUAGGCCCGAA ACAGAGCC | 10383 | |
| 5842 | GCUCUGUUU GAUGCUAU | 965 | AUAGCAUC CUGAUGAGGCCGUUAGGCCCGAA AACAGAGC | 10384 | |
| 5849 | UUGAUGCUA UUUAUGCA | 966 | UGCAUAAA CUGAUGAGGCCGUUAGGCCCGAA AGCAUCAA | 10385 | |
| 5851 | GAUGCUAUU UAUGCAAG | 967 | CUUGCAUA CUGAUGAGGCCGUUAGGCCCGAA AUAGCAUC | 10386 | |
| 5852 | AUGCUAUUU AUGCAAGU | 968 | ACUUGCAU CUGAUGAGGCCGUUAGGCCCGAA AAUAGCAU | 10387 | |
| 5853 | UGCUAUUUA UGCAAGUU | 969 | AACUUGCA CUGAUGAGGCCGUUAGGCCCGAA AAAUAGCA | 10388 | |
| 5861 | AUGCAAGUU AGGGUCUA | 970 | UAGACCCU CUGAUGAGGCCGUUAGGCCCGAA ACUUCCAU | 10389 | |
| 5862 | UGCAAGUUA GGGUCUAU | 971 | AUAGACCC CUGAUGAGGCCGUUAGGCCCGAA AACUUGCA | 10390 | |
| 5867 | GUUAGGGUC UAUGUAUU | 972 | AAUACAUA CUGAUGAGGCCGUUAGGCCCGAA ACCCUAAC | 10391 | |
| 5869 | UAGGGUCUA UGUAUUUA | 973 | UAAAUACA CUCAUGAGCCCGUUAGGCCCGAA AGACCCUA | 10392 | |
| 5873 | GUCUAUGUA UUUAGGAU | 974 | AUCCUAAA CUGAUGAGGCCGUUAGGCCCGAA ACAUAGAC | 10393 | |
| 5875 | CUAUCUAUU UAGGAUGU | 979 | ACAUCCUA CUGAUGAGGCCGUUAGGCCCGAA AUACAUAG | 10398 | |
| 5876 | UAUGUAUUU AGGAUGUC | 980 | GACAUCCU CUGAUGAGGCCGUUAGGCCCGAA AAUACAUA | 10399 | |
| 5877 | AUGUAUUUA GGAUGUCU | 981 | AGACAUCC CUGAUGAGGCCGUUAGGCCCGAA AAAUACAU | 10400 | |
| 5884 | UAGGAUGUC UCCACCUC | 982 | AAGGUGCA CUGAUGAGGCCGUUAGGCCCGAA ACAUCCUA | 10401 | |
| 5892 | CUGCACCUU CUGCAGCC | 983 | GGCUGCAG CUGAUGAGGCCGUUAGGCCCGAA AGGUGCAG | 10402 | |
| 5893 | UGCACCUUC UGCAGCCA | 984 | UGGCUGCA CUGAUGAGGCCGUUAGGCCCGAA AAGGUGCA | 10403 | |
| 5904 | CAGCCAGUC AGAAGCUG | 985 | CAGCUUCU CUGAUGAGGCCGUUAGGCCCGAA ACUGGCUG | 10404 | |
| 5930 | CAGUGGAUC GCUGCUUC | 986 | GAAGCAGC CUGAUGAGGCCGUUAGGCCCGAA AUCCACUG | 10405 | |
| 5937 | UUGCUGCUU CUUGGGGA | 987 | UCCCCAAG CUGAUGAGGCCGUUAGGCCCGAA AGCAGCAA | 10406 | |
| 5938 | UGCUGCUUC UUGGGGAG | 988 | CUCCCCAA CUGAUGAGGCCGUUAGGCCCGAA AAGCAGCA | 10407 | |
| 5940 | CUGCUUCUU GGGGAGAA | 989 | UUCUCCCC CUGAUGAGGCCGUUAGGCCCGAA AGAAGCAG | 10408 | |
| 5953 | AGAAGAGUA UGCUUCCU | 990 | AGGAAGCA CUGAUGAGGCCGUUAGGCCCGAA ACUCUUCU | 10409 | |
| 5958 | AGUAUGCUU CCUUUUAU | 991 | AUAAAAGG CUGAUGAGGCCGUUAGGCCCGAA AGCAUACU | 10410 | |
| 5959 | GUAUGCUCC CUUUUAUC | 992 | GAUAAAAG CUGAUGAGGCCGUUAGGCCCGAA AAGCAUAC | 10411 | |
| 5962 | UGCUUCCUU UUAUCCAU | 993 | AUGGAUAA CUGAUGAGGCCGUUAGGCCCGAA AGGAAGCA | 10412 | |
| 5963 | GCUUCCUUU UAUCCAUG | 994 | CAUGGAUA CUGAUGAGGCCGUUAGGCCCGAA AAGGAAGC | 10413 | |
| 5964 | CUUCCUUUU AUCCAUGU | 995 | ACAUGGAU CUGAUGAGGCCGUUAGGCCCGAA AAAGGAAG | 10414 | |
| 5965 | UUCCUUUUA UCCAUGUA | 996 | UACAUGGA CUGAUGAGGCCGUUAGGCCCGAA AAAAGGAA | 10415 | |
| 5967 | CCUCUUAUC CAUGUAAU | 997 | AUUACAUG CUGAUGAGGCCGUUAGGCCCGAA AUAAAAGG | 10416 | |
| 5973 | AUCCAUGUA AUUUAACU | 998 | AGUUAAAU CUGAUGAGGCCGUUAGGCCCGAA ACAUGGAU | 10417 | |
| 5976 | CAUGUAAUU UAACUGUA | 999 | UACAGUUA CUGAUGAGGCCGUUAGGCCCGAA AUUACAUG | 10418 | |
| 5977 | AUGUAAUUU AACUGUAG | 1000 | CUACAGUU CUGAUGAGGCCGUUAGGCCCGAA AAUUACAU | 10419 | |
| 5978 | UGUAAUUUA ACUGUAGA | 1001 | UCUACAGU CUGAUGAGGCCGUUAGGCCCGAA AAAUUACA | 10420 | |
| 5984 | UUAACUGUA GAACCUGA | 1002 | UCAGGUUC CUGAUGAGGCCGUUAGGCCCGAA ACAGUUAA | 10421 | |
| 5996 | CCUGAGCUC UAAGUAAC | 1003 | GUUACUUA CUGAUGAGGCCGUUAGGCCCGAA AGCUCAGG | 10422 | |
| 5998 | UGAGCUCUA AGUAACCG | 1004 | CGGUUACU CUGAUGAGGCCGUUAGGCCCGAA AGAGCUCA | 10423 | |
| 6002 | CUCUAAGUA ACCGAAGA | 1005 | UCUUCGGU CUGAUGAGGCCGUUAGGCCCGAA ACUCAGAG | 10424 | |
| 6015 | AAGAAUGUA UGCCUCUG | 1006 | CAGAGGCA CUGAUGAGGCCGUUAGGCCCGAA ACAUUCUU | 10425 | |
| 6021 | GUAUGCCUC UGUUCUUA | 1007 | UAAGAACA CUGAUGAGGCCGUUAGGCCCGAA AGGCAUAC | 10426 | |
| 6025 | GCCUCUGUU CUUAUGUG | 1008 | CACAUAAG CUGAUGAGGCCGUUAGGCCCGAA ACAGAGGC | 10427 | |
| 6026 | CCUCUGUUC UUAUGUGC | 1009 | GCACAUAA CUGAUGAGGCCGUUAGGCCCGAA AACAGAGG | 10428 | |
| 6028 | UCUGUUCUU AUGUGCCA | 1010 | UGGCACAU CUGAUGAGGCCGUUAGGCCCGAA AGAACAGA | 10429 | |
| 6029 | CUGUUCUUA UGUGCCAC | 1011 | GUGGCACA CUGAUGAGGCCGUUAGGCCCGAA AAGAACAG | 10430 | |
| 6040 | UGCCACAUC CUUGUUUA | 1012 | UAAACAAG CUGAUGAGGCCGUUAGGCCCGAA AUGUGGCA | 10431 | |
| 6043 | CACAUCCUC GUUUAAAG | 1013 | CUUUAAAC CUGAUGAGGCCGUUAGGCCCGAA AGGAUGUG | 10432 | |
| 6046 | AUCCUUGUU UAAAGGCU | 1014 | AGCCUUUA CUGAUGAGGCCGUUAGGCCCGAA ACAAGGAU | 10433 | |
| 6047 | UCCUUGUUU AAAGGCUC | 1015 | GAGCCUUU CUGAUGAGGCCGUUAGGCCCGAA AACAAGGA | 10434 | |
| 6048 | CCUUGUUUA AAGGCUCU | 1016 | AGAGCCUU CUGAUGAGGCCGUUAGGCCCGAA AAACAAGG | 10435 | |
| 6055 | UAAAGGCUC UCUGUAUG | 1017 | CAUACAGA CUGAUGAGGCCGUUAGGCCCGAA AGCCUUUA | 10436 | |
| 6057 | AAGGCUCUC UGUAUGAA | 1018 | UUCAUACA CUGAUGAGGCCGUUAGGCCCGAA AGAGCCUU | 10437 | |
| 6061 | CUCUCUGUA UGAAGAGA | 1019 | UCUCUUCA CUGAUGAGGCCGUUAGGCCCGAA ACAGAGAG | 10438 | |
| 6079 | GGGACCGUC AUCAGCAC | 1020 | GUGCUGAU CUGAUGAGGCCGUUAGGCCCGAA ACGGUCCC | 10439 | |
| 6082 | ACCGUCAUC AGCACAUU | 1021 | AAUGUGCU CUGAUGAGGCCGUUAGGCCCGAA AUGACGGU | 10440 | |
| 6090 | CAGCACAUU CCCUAGUG | 1022 | CACUAGGG CUGAUGAGGCCGUUAGGCCCGAA AUGUOCUG | 10441 | |
| 6091 | AGCACAUUC CCUAGUGA | 1023 | UCACUAGG CUGAUGAGGCCGUUAGGCCCGAA AAUGUGCU | 10442 | |
| 6095 | CAUUCCCUA GUGAGCCU | 1024 | AGGCUCAC CUGAUGAGGCCGUUAGGCCCGAA AGGGAAUG | 10443 | |
| 6104 | GUGAGCCUA CUGGCUCC | 1025 | GGAGCCAG CUGAUGAGGCCGUUAGGCCCGAA AGGCUCAC | 10444 | |
| 6111 | UACUGGCUC CUGGCAGC | 1026 | GCUGCCAG CUGAUGAGGCCGUUAGGCCCGAA AGCCAGUA | 10445 | |
| 6124 | CAGCGGCUU UUGUGGAA | 1027 | UUCCACAA CUCAUGAGGCCGUUAGGCCCGAA AGCCGCUG | 10446 | |
| 6125 | AGCGGCUUU UGUGGAAG | 1028 | CUUCCACA CUGAUGAGGCCGUUAGGCCCGAA AAGCCGCU | 10447 | |
| 6126 | GCGGCUUUU GUCGAAGA | 1029 | UCUUCCAC CUGAUGAGGCCGUUAGGCCCGAA AAAGCCGC | 10448 | |
| 6137 | GGAAGACUC ACUAGCCA | 1030 | UGGCUAGU CUGAUGAGGCCGUUAGGCCCGAA AGUCUUCC | 10449 | |
| 6141 | GACUCACUA GCCAGAAG | 1031 | CUUCUGGC CUGAUGAGGCCGUUAGGCCCGAA AGUGAGUC | 10450 | |
| 6166 | GGGACAGUC CUCUCCAC | 1032 | GUGGAGAG CUGAUGAGGCCGUUAGGCCCGAA ACUGUCCC | 10451 | |
| 6169 | ACAGUCCUC UCCACCAA | 1033 | UUGGUGGA CUGAUGAGGCCGUUAGGCCCGAA AGGACUGU | 10452 | |
| 6171 | AGUCCUCUC CACCAAGA | 1034 | UCUUGGUG CUGAUGAGGCCGUUAGGCCCGAA AGAGGACU | 10453 | |
| 6181 | ACCAAGAUC UAAAUCCA | 1035 | UGGAUUUA CUGAUGAGGCCGUUAGGCCCGAA AUCUUGGU | 10454 | |
| 6183 | CAAGAUCUA AAUCCAAA | 1036 | UUUGGAUU CUGAUGAGGCCGUUAGGCCCGAA AGAUCUUG | 10455 | |
| 6187 | AUCUAAAUC CAAACAAA | 1037 | UUUGUUUG CUGAUGAGGCCGUUAGGCCCGAA AUUUAGAU | 10456 | |
| 6204 | AGCAGGCUA GAGCCAGA | 1038 | UCUGGCUC CUGAUGAGGCCGUUAGGCCCGAA AGCCUGCU | 10457 | |
| 6226 | GGACAAAUC UUUGUUGU | 1039 | ACAACAAA CUGAUGAGGCCGUUAGGCCCGAA AUUUGUCC | 10458 | |
| 6228 | ACAAAUCUU UGUUGUUC | 1040 | GAACAACA CUGAUGAGGCCGUUAGGCCCGAA AGAUUUGU | 10459 | |
| 6229 | CAAAUCUUU GUUGUUCC | 1041 | GGAACAAC CUGAUGAGGCCGUUAGGCCCGAA AAGAUUUG | 10460 | |
| 6232 | AUCUUUGUU GUUCCUCU | 1042 | AGAGGAAC CUGAUGAGGCCGUUAGGCCCGAA ACAAAGAU | 10461 | |
| 6235 | UUUGUUGUU CCUCUUCU | 1043 | AGAAGAGG CUGAUGAGGCCGUUAGGCCCGAA ACAACAAA | 10462 | |
| 6236 | UUGUUGUUC CUCUUCUU | 1044 | AAGAAGAG CUGAUGAGGCCGUUAGGCCCGAA AACAACAA | 10463 | |
| 6239 | UUGUUCCUC UUCUUUAC | 1045 | GUAAAGAA CUGAUGAGGCCGUUAGGCCCGAA AGGAACAA | 10464 | |
| 6241 | GUUCCUCUU CUUUACAC | 1046 | GUGUAAAG CUGAUGAGGCCGUUAGGCCCGAA AGAGGAAC | 10465 | |
| 6242 | UUCCUCUUC UUUACACA | 1047 | UGUGUAAA CUGAUGAGGCCGUUAGGCCCGAA AAGAGGAA | 10466 | |
| 6244 | CCUCUUCUU UACACAUA | 1048 | UAUGUGUA CUGAUGAGGCCGUUAGGCCCGAA AGAAGAGG | 10467 | |
| 6245 | CUCUUCUUU ACACAUAC | 1049 | GUAUGUGU CUGAUGAGGCCGUUAGGCCCGAA AAGAAGAG | 10468 | |
| 6246 | UCUUCUUUA CACAUACG | 1050 | CGUAUGUG CUGAUGAGGCCGUUAGGCCCGAA AAAGAAGA | 10469 | |
| 6252 | UUACACAUA CGCAAACC | 1051 | GGUUUGCG CUGAUGAGGCCGUUAGGCCCGAA AUGUGUAA | 10470 | |
| 6280 | CUGGCAAUU UUAUAUAU | 1052 | AUUUAUAA CUGAUGAGGCCGUUAGGCCCGAA AUUGCCAG | 10471 | |
| 6281 | UGGCAAUUU UAUAAAUC | 1053 | GAUUUAUA CUGAUGAGGCCGUUAGGCCCGAA AAUUGCCA | 10472 | |
| 6282 | GGCAAUUUU AUAAAUCA | 1054 | UGAUUUAU CUGAUGAGGCCGUUAGGCCCGAA AAAUUGCC | 10473 | |
| 6283 | GCAAUUUUA UAAAUCAG | 1055 | CUGAUUUA CUGAUGAGGCCGUUAGGCCCGAA AAAAUUGC | 10474 | |
| 6285 | AAUUUUAUA AAUCAGGU | 1056 | ACCUGAUU CUGAUGAGGCCGUUAGGCCCGAA AUAAAAUU | 10475 | |
| 6289 | UUAUAAAUC AGGUAACU | 1057 | AGUUACCU CUGAUGAGGCCGUUAGGCCCGAA AUUUAUAA | 10476 | |
| 6294 | AAUCAGGUA ACUGGAAG | 1058 | CUUCCAGU CUGAUGAGGCCGUUAGGCCCGAA ACCUGAUU | 10477 | |
| 6308 | AAGGAGGUU AAACUCAG | 1059 | CUGAGUUU CUGAUGAGGCCGUUAGGCCCGAA ACCUCCUU | 10478 | |
| 6309 | AGGAGGUUA AACUCAGA | 1060 | UCUGAGUU CUGAUGAGGCCGUUAGGCCCGAA AACCUCCU | 10479 | |
| 6314 | GUUAAACUC AGAAAAAA | 1061 | UUUUUUCU CUGAUGAGGCCGUUAGGCCCGAA AGUUUAAC | 10480 | |
| 6331 | GAAGACCUC AGUCAAUU | 1062 | AAUUGACU CUGAUGAGGCCGUUAGGCCCGAA AGGUCUUC | 10481 | |
| 6335 | ACCUCAGUC AAUUCUCU | 1063 | AGAGAAUU CUGAUGAGGCCGUUAGGCCCGAA ACUGAGGU | 10482 | |
| 6339 | CAGUCAAUU CUCUACUU | 1064 | AAGUAGAG CUGAUGAGGCCGUUAGGCCCGAA AUUGACUG | 10483 | |
| 6340 | AGUCAAUUC UCUACUUU | 1065 | AAAGUAGA CUGAUGAGGCCGUUAGGCCCGAA AAUUGACU | 10484 | |
| 6342 | UCAAUUCUC UACUUUUU | 1066 | AAAAAGUA CUGAUGAGGCCGUUAGGCCCGAA AGAAUUGA | 10485 | |
| 6344 | AAUUCUCUA CUUUUUUU | 1067 | AAAPAAAG CUGAUGAGGCCGUUAGGCCCGAA AGAGAAUU | 10486 | |
| 6347 | UCUCUACUU UUUUUUUU | 1068 | AAAAAAAA CUGAUGAGGCCGUUAGGCCCGAA AGUAGAGA | 10487 | |
| 6348 | CUCUACUUU UUUUUUUU | 1069 | AAAAAAAA CUGAUGAGGCCGUUAGGCCCGAA AAGUAGAG | 10488 | |
| 6349 | UCUACUUUU UUUUUUUU | 1070 | AAAAAAAA CUGAUGAGGCCGUUAGGCCCGAA AAAGUAGA | 10489 | |
| 6350 | CUACUUUUU UUUUUUUU | 1071 | AAAAAAAA CUGAUGAGGCCGUUAGGCCCGAA AAAAGUAG | 10490 | |
| 6351 | UACUUUUUU UUUUUUUU | 1072 | AAAAAAAA CUGAUGAGGCCGUUAGGCCCGAA AAAAAGUA | 10491 | |
| 6352 | ACUUUUUUU UUUUUUUU | 1073 | AAAAAAAA CUGAUGAGGCCGUUAGGCCCGAA AAAAAAGU | 10492 | |
| 6353 | CUUUUUUUU UUUUUUUU | 1074 | AAAAAAAA CUGAUGAGGCCGUUAGGCCCGAA AAAAAAAG | 10493 | |
| 6354 | UUUUUUUUU UUUUUUUC | 1075 | CAAAAAAA CUGAUGAGGCCGUUAGGCCCGAA AAAAAAAA | 10494 | |
| 6355 | UUUUUUUUU UUUUUUCC | 1076 | GGAAAAAA CUGAUGAGGCCGUUAGGCCCGAA AAAAAAAA | 10495 | |
| 6356 | UUUUUUUUU UUUUUCCA | 1077 | UGGAAAAA CUGAUGAGGCCGUUAGGCCCGAA AAAAAAAA | 10496 | |
| 6357 | UUUUUUUUU UUUUCCAA | 1078 | UUGGAAAA CUGAUGAGGCCGUUAGGCCCGAA AAAAAAAA | 10497 | |
| 6358 | UUUUUUUUU UUUCCAAA | 1079 | UUUGGAAA CUCAUGAGGCCGUUAGGCCCGAA AAAAAAAA | 10498 | |
| 6359 | UUUUUUUUU UUCCAAAU | 1080 | AUUUGCAA CUGAUGAGGCCGUUAGGCCCGAA AAAAAAAA | 10499 | |
| 6360 | UUUUUUUUU UCCAAAUC | 1081 | GAUUUGGA CUGAUGAGGCCGUUAGGCCCGAA AAAAAAAA | 10500 | |
| 6361 | UUUUUUUUU CCAAAUCA | 1082 | UGAUUUGG CUGAUGAGGCCGUUAGGCCCGAA AAAAAAAA | 10501 | |
| 6362 | UUUUUUUUC CAAAUCAG | 1083 | CUGAUUUG CUGAUGAGCCCGUUAGGCCCGAA AAAAAAAA | 10502 | |
| 6368 | UUCCAAAUC AGAUAAUA | 1084 | UAUUAUCU CUGAUGAGGCCGUUAGGCCCGAA AUUUGGAA | 10503 | |
| 6373 | AAUCAGAUA AUAGCCCA | 1085 | UGGGCUAU CUGAUGAGGCCGUUAGGCCCGAA AUCUGAUU | 10504 | |
| 6376 | CAGAUAAUA GCCCAGCA | 1086 | UGCUGGGC CUGAUGAGGCCGUUAGGCCCGAA AUUAUCUG | 10505 | |
| 6388 | CAGCAAAUA GUGAUAAC | 1087 | GUUAUCAC CUGAUGAGGCCGUUAGGCCCGAA AUUUGCUG | 10506 | |
| 6394 | AUAGUGAUA ACAAAUAA | 1088 | UUAUUUGU CUGAUGAGGCCGUUAGGCCCGAA AUCACUAU | 10507 | |
| 6401 | UAACAAAUA AAACCUUA | 1089 | UAAGGUUU CUGAUGAGGCCGUUAGGCCCGAA AUUUGUUA | 10508 | |
| 6408 | UAAAACCUU AGCUGUUC | 1090 | GAACAGCU CUGAUGAGGCCGUUAGGCCCGAA AGGUUUUA | 10509 | |
| 6409 | AAAACCUUA GCUGUUCA | 1091 | UGAACAGC CUGAUGAGGCCGUUAGGCCCGAA AAGGUUUU | 10510 | |
| 6415 | UUAGCUGUU CAUGUCUU | 1092 | AAGACAUG CUGAUGAGGCCGUUAGGCCCGAA ACAGCUAA | 10511 | |
| 6416 | UAGCUGUUC AUGUCUUG | 1093 | CAAGACAU CUGAUGAGGCCGUTTAGGCCGAA AACAGCUA | 10512 | |
| 6421 | GUUCAUGUC UUGAUUUC | 1094 | GAAAUCAA CUGAUGAGGCCGUUAGGCCCGAA ACAUGAAC | 10513 | |
| 6423 | UCAUGUCUU GAUUUCAA | 1095 | UUGAAAUC CUGAUGAGGCCGUUAGGCCCGAA AGACAUGA | 10514 | |
| 6427 | GUCUUGAUU UCAAUAAU | 1096 | AUUAUUGA CUGAUGAGGCCGUUAGGCCCGAA AUCAAGAC | 10515 | |
| 6428 | UCUUGAUUU CAAUAAUU | 1097 | AAUUAUUG CUGAUGAGGCCGUUAGGCCCGAA AAUCAAGA | 10516 | |
| 6429 | CUUGAUUUC AAUAAUUA | 1098 | UAAUUAUU CUGAUGAGGCCGUUAGGCCCGAA AAAUCAAG | 10517 | |
| 6433 | AUUUCAAUA AUUAAUUC | 1099 | GAAUUAAU CUGAUGAGGCCGUUAGGCCCGAA AUUGAAAU | 10518 | |
| 6436 | UCAAUAAUU AAUUCUUA | 1100 | UAAGAAUU CUGAUGAGGCCGUUAGGCCCGAA AUUAUUGA | 10519 | |
| 6437 | CAAUAAUUA AUUCUUAA | 1101 | UUAAGAAU CUGAUGAGGCCGUUAGGCCCGAA AAUUAUUG | 10520 | |
| 6440 | UAAUUAAUU CUUAAUCA | 1102 | UGAUUAAG CUGAUCAGGCCGUUAGGCCCGAA AUUAAUUA | 10521 | |
| 6441 | AAUUAAUUC UUAAUCAU | 1103 | AUGAUUAA CUGAUGAGGCCGUUAGGCCCGAA AAUUAAUU | 10522 | |
| 6443 | UUAAUUCUU AAUCAUUA | 1104 | UAAUGAUU CUGAUGAGGCCGUUAGGCCCGAA AGAAUUAA | 10523 | |
| 6444 | UAAUUCUUA AUCAUUAA | 1105 | UUAAUGAU CUGAUGAGGCCGUUAGGCCCGAA AAGAAUUA | 10524 | |
| 6447 | UUCUUAAUC AUUAAGAG | 1106 | CUCUUAAU CUGAUGAGGCCGUUAGGCCCGAA AUUAAGAA | 10525 | |
| 6450 | UUAAUCAUU AAGAGACC | 1107 | GGUCUCUU CUGAUGAGGCCGUUAGGCCCGAA AUGAUUAA | 10526 | |
| 6451 | UAAUCAUUA AGAGACCA | 1108 | UGGUCUCU CUGAUGAGGCCGUUAGGCCCGAA AAUGAUUA | 10527 | |
| 6461 | GAGACCAUA AUAAAUAC | 1109 | GUAUUUAU CUGAUGAGGCCGUUAGGCCCGAA AUGGUCUC | 10528 | |
| 6464 | ACCAUAAUA AAUACUCC | 1110 | GGAGUAUU CUGAUGAGGCCGUUAGGCCCGAA AUUAUGGU | 10529 | |
| 6468 | UAAUAAAUA CUCCUUUU | 1111 | AAAAGGAG CUGAUGAGGCCGUUAGGCCCGAA AUUUAUUA | 10530 | |
| 6471 | UAAAUACUC CUUUUCAA | 1112 | UUGAAAAG CUGAUGAGGCCGUUAGGCCCGAA AGUAUUUA | 10531 | |
| 6474 | AUACUCCUU UUCAAGAG | 1113 | CUCUUGAA CUGAUGAGGCCGUUAGGCCCGAA AGGAGUAU | 10532 | |
| 6475 | UACUCCUUU UCAAGAGA | 1114 | UCUCUUGA CUGAUGAGGCCGUUAGGCCCGAA AAGGAGUA | 10533 | |
| 6476 | ACUCCUUUU CAAGAGAA | 1115 | UUCUCUUG CUGAUGAGGCCGUUAGGCCCGAA AAAGGAGU | 10534 | |
| 6477 | CUCCUUUUC AAGAGAAA | 1116 | UUUCUCUU CUGAUGAGGCCGUUAGGCCCGAA AAAAGGAG | 10535 | |
| 6497 | AAAACCAUU AGAAUUGU | 1117 | ACAAUUCU CUGAUGAGGCCGUUAGGCCCGAA AUGGUUUU | 10536 | |
| 6498 | AAACCAUUA GAAUUGUU | 1118 | AACAAUUC CUGAUGAGGCCGUUAGGCCCGAA AAUGGUUU | 10537 | |
| 6503 | AUUAGAAUU GUUACUCA | 1119 | UGAGUAAC CUGAUGAGGCCGUUAGGCCCGAA AUUCUAAU | 10538 | |
| 6506 | AGAAUUGUU ACUCAGCU | 1120 | AGCUGAGU CUGAUGAGGCCGUUAGGCCCGAA ACAAUUCU | 10539 | |
| 6507 | GAAUUGUUA CUCAGCUC | 1121 | GAGCUGAG CUGAUGAGGCCGUUAGGCCCGAA AACAAUUC | 10540 | |
| 6510 | UUGUUACUC AGCUCCUU | 1122 | AAGGAGCU CUGAUGAGGCCGUUAGGCCCGAA AGUAACAA | 10541 | |
| 6515 | ACUCAGCUC CUUCAAAC | 1123 | GUUUGAAG CUGAUGAGGCCGUUAGGCCCGAA AGCUGAGU | 10542 | |
| 6518 | CAGCUCCUU CAAACUCA | 1124 | UGAGUUUG CUGAUGAGGCCGUUAGGCCCGAA AGGAGCUG | 10543 | |
| 6519 | AGCUCCUUC AAACUCAG | 1125 | CUGAGUUU CUGAUGAGGCCGUUAGGCCCGAA AAGGAGCU | 10544 | |
| 6525 | UUCAAACUC AGGUUUGU | 1126 | ACAAACCU CUGAUGAGGCCGUUAGGCCCGAA AGUUUCAA | 10545 | |
| 6530 | ACUCAGGUU UGUAGCAU | 1127 | AUGCUACA CUGAUGAGGCCGUUAGGCCCGAA ACCUGAGU | 10546 | |
| 6531 | CUCAGGUUU GUAGCAUA | 1128 | UAUGCUAC CUGAUGAGGCCGUUAGGCCCGAA AACCUGAG | 10547 | |
| 6534 | AGGUUUGUA GCAUACAU | 1129 | AUGUAUGC CUGAUGAGGCCGUUAGGCCCGAA ACAAACCU | 10548 | |
| 6539 | UGUAGCAUA CAUGAGUC | 1130 | GACUCAUG CUGAUGAGGCCGUUAGGCCCGAA AUGCUACA | 10549 | |
| 6547 | ACAUGAGUC CAUCCAUC | 1131 | GAUGGAUG CUGAUGAGGCCGUUAGGCCCGAA ACUCAUGU | 10550 | |
| 6551 | GAGUCCAUC CAUCAGUC | 1132 | GACUGAUG CUGAUGAGGCCGUUAGGCCCGAA AUGGACUC | 10551 | |
| 6555 | CCAUCCAUC AGUCAAAG | 1133 | CUUUGACU CUGAUGAGGCCGUUAGGCCCGAA AUGGAUGG | 10552 | |
| 6559 | CCAUCAGUC AAAGAAUG | 1134 | CAUUCUUU CUGAUGAGGCCGUUAGGCCCGAA ACUGAUGG | 10553 | |
| 6570 | AGAAUGGUU CCAUCUGG | 1135 | CCAGAUGG CUGAUGAGGCCGUUAGGCCCGAA ACCAUUCU | 10554 | |
| 6571 | GAAUGGUUC CAUCUGGA | 1136 | UCCAGAUG CUGAUGAGGCCGUUAGGCCCGAA AACCAUUC | 10555 | |
| 6575 | GGUUCCAUC UGGAGUCU | 1137 | AGACUCCA CUGAUGAGGCCGUUAGGCCCGAA AUGGAACC | 10556 | |
| 6582 | UCUGGAGUC UUAAUGUA | 1138 | UACAUUAA CUGAUGAGGCCGUUAGGCCCGAA ACUCCAGA | 10557 | |
| 6584 | UGGAGUCUU AAUGUAGA | 1139 | UCUACAUU CUGAUGAGGCCGUUAGGCCCGAA AGACUCCA | 10558 | |
| 6585 | GGAGUCUUA AUGUAGAA | 1140 | UUCUACAU CUGAUGAGGCCGUUAGGCCCGAA AAGACUCC | 10559 | |
| 6590 | CUUAAUGUA GAAAGAAA | 1141 | UUUCUUUC CUGAUGAGGCCGUUAGGCCCGAA ACAUUAAG | 10560 | |
| 6609 | UGGAGACUU GUAAUAAU | 1142 | AUUAUUAC CUGAUGAGGCCGUUAGGCCCGAA AGUCUCCA | 10561 | |
| 6612 | AGACUUGUA AUAAUGAG | 1143 | CUCAUUAU CUGAUGAGGCCGUUAGGCCCGAA ACAAGUCU | 10562 | |
| 6615 | CUUGUAAUA AUGAGCUA | 1144 | UAGCUCAU CUGAUGAGGCCGUUAGGCCCGAA AUUACAAG | 10563 | |
| 6623 | AAUGAGCUA GUUACAAA | 1145 | UUUGUAAC CUGAUGAGGCCGUUAGGCCCGAA AGCUCAUU | 10564 | |
| 6626 | GAGCUAGUU ACAAAGUG | 1146 | CACUUUGU CUGAUGAGGCCGUUAGGCCCGAA ACUAGCUC | 10565 | |
| 6627 | AGCUAGUUA CAAAGUGC | 1147 | GCACUUUG CUGAUGAGGCCGUUAGGCCCGAA AACUAGCU | 10566 | |
| 6637 | AAAGUGCUU GUUCAUUA | 1148 | UAAUGAAC CUGAUGAGGCCGUUAGGCCCGAA AGCACUUU | 10567 | |
| 6640 | GUGCUUGUU CAUUAAAA | 1149 | UUUUAAUG CUGAUGAGGCCGUUAGGCCCGAA ACAAGCAC | 10568 | |
| 6641 | UGCUUGUUC AUUAAAAU | 1150 | AUUUUAAU CUGAUGAGGCCGUUAGGCCCGAA AACAAGCA | 10569 | |
| 6644 | UUGUUCAUU AAAAUAGC | 1151 | GCUAUUUU CUGAUGAGGCCGUUAGGCCCGAA AUGAACAA | 10570 | |
| 6645 | UGUUCAUUA AAAUAGCA | 1152 | UGCUAUUU CUGAUGAGGCCGUUAGGCCCGAA AAUGAACA | 10571 | |
| 6650 | AUUAAAAUA GCACUGAA | 1153 | UUCAGUGC CUGAUGAGGCCGUUAGGCCCGAA AUUUUAAU | 10572 | |
| 6662 | CUGAAAAUU GAAACAUG | 1154 | CAUGUUUC CUGAUGAGGCCGUUAGGCCCGAA AUUUUCAG | 10573 | |
| 6674 | ACAUGAAUU AACUGAUA | 1155 | UAUCAGUU CUGAUGAGGCCGUUAGGCCCGAA AUUCAUGU | 10574 | |
| 6675 | CAUGAAUUA ACUGAUAA | 1156 | UUAUCAGU CUGAUGAGGCCGUUAGGCCCGAA AAUUCAUG | 10575 | |
| 6682 | UAACUGAUA AUAUUCCA | 1157 | UGGAAUAU CUGAUGAGGCCGUUAGGCCCGAA AUCAGUUA | 10576 | |
| 6685 | CUGAUAAUA UUCCAAUC | 1158 | GAUUGGAA CUGAUGAGGCCGUUAGGCCCGAA AUUAUCAG | 10577 | |
| 6687 | GAUAAUAUU CCAAUCAU | 1159 | AUGAUUGG CUGAUGAGGCCGUUAGGCCCGAA AUAUUAUC | 10578 | |
| 6688 | AUAAUAUUC CAAUCAUU | 1160 | AAUGAUUG CUGAUGAGGCCGUUAGGCCCGAA AAUAUUAU | 10579 | |
| 6693 | AUUCCAAUC AUUUGCCA | 1161 | UGGCAAAU CUGAUGAGGCCGUUAGGCCCGAA AUUGGAAU | 10580 | |
| 6696 | CCAAUCAUU UGCCAUUU | 1162 | AAAUGGCA CUGAUGAGGCCGUUAGGCCCGAA AUGAUUGG | 10581 | |
| 6697 | CAAUCAUUU GCCAUUUA | 1163 | UAAAUGGC CUGAUGAGGCCGUUAGGCCCGAA AAUGAUUG | 10582 | |
| 6703 | UUUGCCAUU UAUGACAA | 1164 | UUGUCAUA CUGAUGAGGCCGUUAGGCCCGAA AUGGCAAA | 10583 | |
| 6704 | UUGCCAUUU AUGACAAA | 1165 | UUUGUCAU CUGAUGAGGCCGUUAGGCCCGAA AAUGGCAA | 10584 | |
| 6705 | UGCCAUUUA UGACAAAA | 1166 | UUUUGUCA CUGAUGAGGCCGUUAGGCCCGAA AAAUGGCA | 10585 | |
| 6719 | AAAAUGGUU GGCACUAA | 1167 | UUAGUGCC CUGAUGAGGCCGUUAGGCCCGAA ACCAUUUU | 10586 | |
| 6726 | UUGGCACUA ACAAAGAA | 1168 | UUCUUUGU CUGAUGAGGCCGUUAGGCCCGAA AGUGCCAA | 10587 | |
| 6743 | CGAGCACUU CCUUUCAG | 1169 | CUGAAAGG CUGAUGAGGCCGUUAGGCCCGAA AGUGCUCG | 10588 | |
| 6744 | GAGCACUUC CUUUCAGA | 1170 | UCUGAAAG CUGAUGAGGCCGUUAGGCCCGAA AAGUGCUC | 10589 | |
| 6747 | CACUUCCUU UCAGAGUU | 1171 | AACUCUGA CUGAUGAGGCCGUUAGGCCCGAA AGGAAGUG | 10590 | |
| 6748 | ACUUCCUUU CAGAGUUU | 1172 | AAACUCUG CUGAUGAGGCCGUUAGGCCCGAA AAGGAAGU | 10591 | |
| 6749 | CUUCCUUUC AGACUUUC | 1173 | GAAACUCU CUGAUGAGGCCGUUAGGCCCGAA AAAGGAAG | 10592 | |
| 6755 | UUCAGAGUU UCUCAGAU | 1174 | AUCUCAGA CUGAUGAGGCCGUUAGGCCCGAA ACUCUGAA | 10593 | |
| 6756 | UCAGAGUUU CUGAGAUA | 1175 | UAUCUCAG CUGAUGAGGCCGUUAGGCCCGAA AACUCUGA | 10594 | |
| 6757 | CAGAGUUUC UGAGAUAA | 1176 | UUAUCUCA CUGAUGAGGCCGUUAGGCCCGAA AAACUCUG | 10595 | |
| 6764 | UCUGAGAUA AUGUACGU | 1177 | ACGUACAU CUGAUGAGGCCGUUAGGCCCGAA AUCUCACA | 10596 | |
| 6769 | GAUAAUGUA CGUGGAAC | 1178 | GUUCCACG CUGAUGAGGCCGUUAGGCCCGAA ACAUUAUC | 10597 | |
| 6781 | GGAACAGUC UGOGUGGA | 1179 | UCCACCCA CUGAUGAGGCCGUUAGGCCCGAA ACUGUUCC | 10598 | |
| 6814 | GUGCAAGUC UGUGUCUU | 1180 | AAGACACA CUGAUGAGGCCGUUAGGCCCGAA ACUUGCAC | 10599 | |
| 6820 | GUCUGUGUC UUGUCAGU | 1181 | ACUGACAA CUGAUGAGGCCGUUAGGCCCGAA ACACAGAC | 10600 | |
| 6822 | CUGUGUCUU GUCAGUCC | 1182 | GGACUGAC CUGAUGAGGCCGUUAGGCCCGAA AGACACAG | 10601 | |
| 6825 | UGUCUGGUC AGUCCAAG | 1183 | CUUGGACU CUGAUGAGGCCGUUAGGCCCGAA ACAAGACA | 10602 | |
| 6829 | UUGUCAGUC CAAGAAGU | 1184 | ACUUCUUG CUGAUGAGGCCGUUAGGCCCGAA ACUGACAA | 10603 | |
| 6851 | CGAGAUGUU AAUUUUAG | 1185 | CUAAAAUU CUGAUGAGGCCGUUAGGCCCGAA ACAUCUCG | 10604 | |
| 6852 | GAGAUGUUA AUUUUAGG | 1186 | CCUAAAAU CUGAUGAGGCCGUUAGGCCCGAA AACAUCUC | 10605 | |
| 6855 | AUGUUAAUU UUAGGGAC | 1187 | GUCCCUAA CUGAUGAGGCCGUUAGGCCCGAA AUUAACAU | 10606 | |
| 6856 | UGUUAAUUU UAGGGACC | 1188 | GGUCCCUA CUGAUGAGGCCGUUAGGCCCGAA AAUUAACA | 10607 | |
| 6857 | GUUAAUUUU AGGGACCC | 1189 | GGGUCCCU CUGAUGAGGCCGUUAGGCCCGAA AAAUUAAC | 10608 | |
| 6858 | UUAAUUUUA GGGACCCG | 1190 | CGGGUCCC CUGAUGAGGCCGUUAGGCCCGAA AAAAUUAA | 10609 | |
| 6872 | CCGUGCCUU GUUUCCUA | 1191 | UAGGAAAC CUGAUGAGGCCGUUAGGCCCGAA AGGCACGG | 10610 | |
| 6875 | UGCCUUGUU UCCUAGCC | 1192 | GGCUAGGA CUGAUGAGGCCGUUAGGCCCGAA ACAAGGCA | 10611 | |
| 6876 | GCCUUGUUU CCUAGCCC | 1193 | GGGCUAGG CUGAUGAGGCCGUUAGGCCCGAA AACAAGGC | 10612 | |
| 6877 | CCUUGUUUC CUAGCCCA | 1194 | UGGGCUAG CUGAUGAGGCCGUUAGGCCCGAA AAACAAGG | 10613 | |
| 6880 | UGUUUCCUA GCCCACAA | 1195 | UUGUGGGC CUGAUGAGGCCGUUAGGCCCGAA AGGAAACA | 10614 | |
| 6901 | GCAAACAUC AAACAGAU | 1196 | AUCUGUUU CUGAUGAGGCCGUUAGGCCCGAA AUGUUUGC | 10615 | |
| 6910 | AAACAGAUA CUCGCUAG | 1197 | CUAGCGAG CUGAUGAGGCCGUUAGGCCCGAA AUCUGUUU | 10616 | |
| 6913 | CAGAUACUC GCUAGCCU | 1198 | AGGCUAGC CUGAUGAGGCCGUUAGGCCCGAA AGUAUCUG | 10617 | |
| 6917 | UACUCGCUA GCCUCAUU | 1199 | AAUGAGGC CUGAUGAGGCCGUUAGGCCCGAA AGCGAGUA | 10618 | |
| 6922 | GCUAGCCUC AUUUAAAU | 1200 | AUUUAAAU CUGAUGAGGCCGUUAGGCCCGAA AGGCUAGC | 10619 | |
| 6925 | AGCCUCAUU UAAAUUGA | 1201 | UCAAUUUA CUGAUGAGGCCGUUAGGCCCGAA AUGAGGCU | 10620 | |
| 6926 | GCCUCAUUU AAAUUGAU | 1202 | AUCAAUUU CUGAUGAGGCCGUUAGGCCCGAA AAUGAGGC | 10621 | |
| 6927 | CCUCAUUUA AAUUGAUU | 1203 | AAUCAAUU CUGAUGAGGCCGUUAGGCCCGAA AAAUGAGG | 10622 | |
| 6931 | AUUUAAAUU GAUUAAAG | 1204 | CUUUAAUC CUGAUGAGGCCGUUAGGCCCGAA AUUUAAAU | 10623 | |
| 6935 | AAAUUGAUU AAAGGAGG | 1205 | CCUCCUUU CUGAUGAGGCCGUUAGGCCCGAA AUCAAUUU | 10624 | |
| 6936 | AAUUGAUUA AAGGAGGA | 1206 | UCCUCCUU CUGAUGAGGCCGUUAGGCCCGAA AAUCAAUU | 10625 | |
| 6951 | GAGUGCAUC UUUGGCCG | 1207 | CGGCCAAA CUGAUGAGGCCGUUAGGCCCGAA AUGCACUC | 10626 | |
| 6953 | GUGCAUCUU UGGCCGAC | 1208 | GUCGGCCA CUGAUGAGGCCGUUAGGCCCGAA AGAUGCAC | 10627 | |
| 6954 | UGCAUCUUU GGCCGACA | 1209 | UGUCGGCC CUGAUGAGGCCGUUAGGCCCGAA AAGAUGCA | 10628 | |
| 6970 | AGUGGUGUA ACUGUGUG | 1210 | CACACAGU CUGAUGAGGCCGUUAGGCCCGAA ACACCACU | 10629 | |
| 7026 | GUGGGUGUA UGUGUGUG | 1211 | AACACACA CUGAUGAGGCCGUUAGGCCCGAA ACACCCAC | 10630 | |
| 7034 | AUGUGUGUU UUGUGCAU | 1212 | AUGCACAA CUGAUGAGGCCGUUAGGCCCGAA ACACACAU | 10631 | |
| 7035 | UGUGUGUUU UGUGCAUA | 1213 | UAUGCACA CUGAUGAGGCCGUUAGGCCCGAA AACACACA | 10632 | |
| 7036 | GUGUGUUUU GUGCAUAA | 1214 | UUAUGCAC CUGAUGAGGCCGUUAGGCCCGAA AAACACAC | 10633 | |
| 7043 | UUGUGCAUA ACUAUUUA | 1215 | UAAAUAGU CUGAUGAGGCCGUUAGGCCCGAA AUGCACAA | 10634 | |
| 7047 | GCAUAACUA UUUAAGGA | 1216 | UCCUUAAA CUGAUGAGGCCGUUAGGCCCGAA AGUUAUGC | 10635 | |
| 7049 | AUAACUAUU UAAGGAAA | 1217 | UUUCCUUA CUGAUGAGGCCGUUAGGCCCGAA AUAGUUAU | 10636 | |
| 7050 | UAACUAUUU AAGGAAAC | 1218 | GUUUCCUU CUGAUGAGGCCGUUAGGCCCGAA AAUAGUUA | 10637 | |
| 7051 | AACUAUUUA AGGAAACU | 1219 | AGUUUCCU CUGAUGAGGCCGUUAGGCCCGAA AAAUAGUU | 10638 | |
| 7065 | ACUGGAAUU UUAAAGUU | 1220 | AACUUUAA CUGAUGAGGCCGUUAGGCCCGAA AUUCCAGU | 10639 | |
| 7066 | CUGGAAUUU UAAAGUUA | 1221 | UAACUUUA CUGAUGAGGCCGUUAGGCCCGAA AAUUCCAG | 10640 | |
| 7067 | UGGAAUUUU AAAGUUAC | 1222 | GUAACUUU CUGAUGAGGCCGUUAGGCCCGAA AAAUUCCA | 10641 | |
| 7068 | GGAAUUUUA AAGUUACU | 1223 | AGUAACUU CUGAUGAGGCCGUUAGGCCCGAA AAAAUUCC | 10642 | |
| 7073 | UUUAAAGUU ACUUUUAU | 1224 | AUAAAAGU CUGAUGAGGCCGUUAGGCCCGAA ACUUUAAA | 10643 | |
| 7074 | UUAAAGUUA CUUUUAUA | 1225 | UAUAAAAG CUGAUGAGGCCGUUAGGCCCGAA AACUUUAA | 10644 | |
| 7077 | AAGUUACUU UUAUACAA | 1226 | UUGUAUAA CUGAUGAGGCCGUUAGGCCCGAA AGUAACUU | 10645 | |
| 7078 | AGUUACUUU UAUACAAA | 1227 | UUUGUAUA CUGAUGAGGCCGUUAGGCCCGAA AAGUAACU | 10646 | |
| 7079 | GUUACUUUU AUACAAAC | 1228 | GUUUGUAU CUGAUGAGGCCGUUAGGCCCGAA AAAGUAAC | 10647 | |
| 7080 | UUACUUUUA UACAAACC | 1229 | GGUUUGUA CUGAUGAGGCCGUUAGGCCCGAA AAAAGUAA | 10648 | |
| 7082 | ACUUUUAUA CAAACCAA | 1230 | UUGCUUUC CUGAUGAGGCCGUUAGGCCCGAA AUAAAAGU | 10649 | |
| 7095 | CCAAGAAUA UAUGCUAC | 1231 | GUAGCAUA CUGAUGAGGCCGUUAGGCCCGAA AUUCUUGG | 10650 | |
| 7097 | AAGAAUAUA UGCUACAG | 1232 | CUCUACCA CUGAUGAGGCCGUUAGGCCCGAA AUAUUCUU | 10651 | |
| 7102 | UAUAUGCUA CAGAUAUA | 1233 | UAUAUCUA CUGAUGAGGCCGUUAGGCCCGAA AGCAUAUA | 10652 | |
| 7108 | CUACAGAUA UAAGACAG | 1234 | CUGUCUUA CUGAUGAGGCCGUUAGGCCCGAA AUCUGUAG | 10653 | |
| 7110 | ACAGAUAUA AGACAGAC | 1235 | GUCUGUCU CUGAUGAGGCCGUUAGGCCCGAA AUAUCUGU | 10654 | |
| 7124 | GACAUGGUU UGGUCCUA | 1236 | UAGGACCA CUGAUGAGGCCGUUAGGCCCGAA ACCAUGUC | 10655 | |
| 7125 | ACAUGGUUU GGUCCUAU | 1237 | AUAGGACC CUGAUGAGGCCGUUAGGCCCGAA AACCAUGU | 10656 | |
| 7129 | GGUUUGGUC CUAUAUUU | 1238 | AAAUAUAG CUGAUGAGGCCGUUAGGCCCGAA ACCAAACC | 10657 | |
| 7132 | UUGGUCCUA UAUUUCUA | 1239 | UAGAAAUA CUGAUGAGGCCGUUAGGCCCGAA AGGACCAU | 10658 | |
| 7134 | GGUCCUAUA UUUCUAGU | 1240 | ACUAGAAA CUGAUGAGGCCGUUAGGCCCGAA AUAGGACC | 10659 | |
| 7136 | UCCUAUAUU UCUAGUCA | 1241 | UGACUAGA CUGAUGAGGCCGUUAGGCCCGAA AUAUAGGA | 10660 | |
| 7137 | CCUAUAUUU CUAGUCAU | 1242 | AUGACUAG CUGAUGAGGCCGUUAGGCCCGAA AAUAUAGG | 10661 | |
| 7138 | CUAUAUUUC GAGUCAUG | 1243 | CAUGACUA CUGAUGAGGCCGUUAGGCCCGAA AAAUAUAG | 10662 | |
| 7140 | AUAUUUCUA GUCAUGAU | 1244 | AUCAUGAC CUGAUGAGGCCGUUAGGCCCGAA AGAAAUAU | 10663 | |
| 7143 | UGUCUAGUC AUGAUGAA | 1245 | UUCAUCAU CUGAUGAGGCCGUUAGGCCCGAA ACUAGAAA | 10664 | |
| 7155 | AUGAAUGUA UUUUGUAU | 1246 | AUACAAAA CUGAUGAGGCCGUUAGGCCCGAA ACAUUCAU | 10665 | |
| 7157 | GAAUGUAUU UGGUAGAC | 1247 | GUAUACAA CUGAUGAGGCCGUUAGGCCCGAA AUACAUUC | 10666 | |
| 7158 | AAUGUAUUU UGUAUACC | 1248 | GGUAUACA CUGAUGAGGCCGUUAGGCCCGAA AAUACAUU | 10667 | |
| 7159 | AUGUAUUUU GUAUACCA | 1249 | UGGUAUAC CUGAUGAGGCCGUUAGGCCCGAA AAAUACAU | 10668 | |
| 7162 | UAUUUUGUA UACCAUCU | 1250 | AGAUGGUA CUGAUGAGGCCGUUAGGCCCGAA ACAAAAUA | 10669 | |
| 7164 | UUUUGUAUA CCAUCUUC | 1251 | GAAGAUGG CUGAUGAGGCCGUUAGGCCCGAA AUACAAAA | 10670 | |
| 7169 | UAUACCAUC UUCAUAUA | 1252 | UAUAUGAA CUGAUGAGGCCGUUAGGCCCGAA AUGGUAUA | 10671 | |
| 7171 | UACCAUCUU CAUAUAAU | 1253 | AUUAUAUG CUGAUGAGGCCGUUAGGCCCGAA AGAUGGUA | 10672 | |
| 7172 | ACCAUCUUC AUAUAAUA | 1254 | UAUUAUAU CUGAUGAGGCCGUUAGGCCCGAA AAGAUGGU | 10673 | |
| 7175 | AUCUUCAUA UAAUAUAC | 1255 | GUAGAUHA CUGAUGAGGCCGUUAGGCCCGAA AUGAAGAU | 10674 | |
| 7177 | CUUCAUAUA AUAUACUU | 1256 | AAGUAUAU CUGAUGAGGCCGUUAGGCCCGAA AUAUGAAG | 10675 | |
| 7180 | CAUAUAAUA UACUUAAA | 1257 | UUUAAGUA CUGAUGAGGCCGUUAGGCCCGAA AUUAUAUG | 10676 | |
| 7182 | UAUAAUAUA CUUAAAAA | 1258 | UUUUUAAG CUGAUGAGGCCGUUAGGCCCGAA AUAUUAUA | 10677 | |
| 7185 | AAUAUACUU AAAAAUAU | 1259 | AUAUUUUU CUGAUGAGGCCGUUAGGCCCGAA AGUAUAUU | 10678 | |
| 7186 | AUAUACUUA AAAAUAUU | 1260 | AAUAUUUU CUGAUGAGGCCGUUAGGCCCGAA AAGUAUAU | 10679 | |
| 7192 | UUAAAAAUA UUUCUUAA | 1261 | UUAAGAAA CUGAUGAGGCCGUUAGGCCCGAA AUUUUUAA | 10680 | |
| 7194 | AAAAAUAUU UCUUAAUU | 1262 | AAUUAAGA CUGAUGAGGCCGUUAGGCCCGAA AUAUUUUU | 10681 | |
| 7195 | AAAAUAUUU CUUAAUUG | 1263 | CAAUUAAG CUGAUGAGGCCGUUAGGCCCGAA AAUAUUUU | 10682 | |
| 7196 | AAAUAUUUC UUAAUUGG | 1264 | CCAAUUAA CUGAUGAGGCCGUUAGGCCCGAA AAAUAUUU | 10683 | |
| 7198 | AUAUUUCUU AAUUGGGA | 1265 | UCCCAAUU CUGAUGAGGCCGUUAGGCCCGAA AGAAAUAU | 10684 | |
| 7199 | UAUUUCUUA AUUGGGAU | 1266 | AUCCCAAU CUGAUGAGGCCGUUAGGCCCGAA AAGAAAUA | 10685 | |
| 7202 | UUCUUAAU | GGGAUUUG | 1267 | CAAAUCCC CUGAUGAGGCCGUUAGGCCCGAA AUUAAGAA | 10686 |
| 7208 | AUUGGGAUU UGUAAUCG | 1268 | CGAUUACA CUGAUGAGGCCGUUAGGCCCGAA AUCCCAAU | 10687 | |
| 7209 | UUGGGAUUU GUAAUCGU | 1269 | ACGAUUAC CUGAUGAGGCCGUUAGGCCCGAA AAUCCCAA | 10688 | |
| 7212 | GGAUUUGUA AUCGUACC | 1270 | GGUACGAU CUGAUGAGGCCGUUAGGCCCGAA ACAAAUCC | 10689 | |
| 7215 | UUUGUAAUC GUACCAAC | 1271 | GUUGGUAC CUGAUGAGGCCGUUAGGCCCGAA AUUACAAA | 10690 | |
| 7218 | GUAAUCGUA CCAACUUA | 1272 | UAAGUUGG CUGAUGAGGCCGUUAGGCCCGAA ACGAUUAC | 10691 | |
| 7225 | UACCAACUU AAUUCAUA | 1273 | UAUCAAUU CUGAUGAGGCCGUUAGGCCCGAA AGUUGGUA | 10692 | |
| 7226 | ACCAACUUA AUUGAUAA | 1274 | UUAUCAAU CUGAUGAGGCCGUUAGGCCCGAA AAGUUGGU | 10693 | |
| 7229 | AACUUAAUU CAUAAACU | 1275 | AGUUUAUC CUGAUGAGGCCGUUAGGCCCGAA AUUAAGUU | 10694 | |
| 7233 | UAAUUGAUA AACUUGGC | 1276 | GCCAAGUU CUGAUGAGGCCGUUAGGCCCGAA AUCAAUUA | 10695 | |
| 7238 | GAUAAACUU GGCAACUG | 1277 | CAGUUGCC CUGAUGAGGCCGUUAGGCCCGAA AGUUUAUC | 10696 | |
| 7249 | CAACUGCUU UUAUGUUC | 1278 | GAACAUAA CUGAUGAGGCCGUUAGGCCCGAA AGCAGUUG | 10697 | |
| 7250 | AACUGCUUU UAUGUUCU | 1279 | AGAACAUA CUGAUGAGGCCGUUAGGCCCGAA AAGCAGUU | 10698 | |
| 7251 | ACUGCUUUU AUGUUCUG | 1280 | CAGAACAU CUGAUGAGGCCGUUAGGCCCGAA AAAGCAGU | 10699 | |
| 7252 | CUGCUUUUA UGUUCUGU | 1281 | ACAGAACA CUGAUGAGGCCGUUAGGCCCGAA AAAAGCAG | 10700 | |
| 7256 | UUUUAUGUU CUGUCUCC | 1282 | GGAGACAG CUGAUGAGGCCGUUAGGCCCGAA ACAUAAAA | 10701 | |
| 7257 | UUUAUGUUC UGUCUCCU | 1283 | AGGAGACA CUGAUGAGGCCGUUAGGCCCGAA AACAUAAA | 10702 | |
| 7261 | UGUUCUGUC UCCUUCCA | 1284 | UGGAAGGA CUGAUGAGGCCGUUAGGCCCGAA ACAGAACA | 10703 | |
| 7263 | UUCUGUCUC CUUCCAUA | 1285 | UAUGGAAG CUGAUGAGGCCGUUAGGCCCGAA AGACAGAA | 10704 | |
| 7266 | UGUCUCCUU CCAUAAAU | 1286 | AUUUAUGG CUGAUGAGGCCGUUAGGCCCGAA AGGAGACA | 10705 | |
| 7267 | GUCUCCUUC CAUAAAUU | 1287 | AAUUUAUG CUGAUGAGGCCGUUAGGCCCGAA AAGGAGAC | 10706 | |
| 7271 | CCUUCCAUA AAUUUUUC | 1288 | GAAAAAUU CUGAUGAGGCCGUUAGGCCCGAA AUGGAAGG | 10707 | |
| 7275 | CCAUAAAUU UUUCAAAA | 1289 | UUUUGAAA CUGAUGAGGCCGUUAGGCCCGAA AUUUAUGG | 10708 | |
| 7276 | CAUAAAUUU UUCAAAAU | 1290 | AUUUUGAA CUGAUGAGGCCGUUAGGCCCGAA AAUUUAUG | 10709 | |
| 7277 | AUAAAUUUU UCAAAAUA | 1291 | UAUUUUGA CUGAUGAGGCCGUUAGGCCCGAA AAAUUUAU | 10710 | |
| 7278 | UAAAUUUUU CAAAAUAC | 1292 | GUAUUUUG CUGAUGAGGCCGUUAGGCCCGAA AAAAUUUA | 10711 | |
| 7279 | AAAUUUUUC AAAAUACU | 1293 | AGUAUUUU CUGAUGAGGCCGUUAGGCCCGAA AAAAAUUU | 10712 | |
| 7285 | UUCAAAAUA CUAAUUCA | 1294 | UGAAUUAG CUGAUGAGGCCGUUAGGCCCGAA AUUUUGAA | 10713 | |
| 7288 | AAAAUACUA AUUCAACA | 1295 | UGUUGAAU CUGAUGAGGCCGUUAGGCCCGAA AGUAUUUU | 10714 | |
| 7291 | AUACUAAUU CAACAAAG | 1296 | CUUUGUUG CUGAUGAGGCCGUUAGGCCCGAA AUUAGUAU | 10715 | |
| 7292 | UACUAAUUC AACAAAGA | 1297 | UCUUUGUU CUGAUGAGGCCGUUAGGCCCGAA AAUUAGUA | 10716 | |
| 7308 | AAAAAGCUC UUUUUUUU | 1298 | AAAAAAAA CUGAUGAGGCCGUUAGGCCCGAA AGCUUUUU | 10717 | |
| 7310 | AAAGCUCUU UUUUUUCC | 1299 | GGAAAAAA CUGAUGAGGCCGUUAGGCCCGAA AGAGCUUU | 10718 | |
| 7311 | AAGCUCUUU UUUUUCCU | 1300 | AGGAAAAA CUGAUGAGGCCGUUAGGCCCGAA AAGAGCUU | 10719 | |
| 7312 | AGCUCUUUU UUUUCCUA | 1301 | UAGGAAAA CUGAUGAGGCCGUUAGGCCCGAA AAAGAGCU | 10720 | |
| 7313 | GCUCUUUUU UUUCCUAA | 1302 | UUAGGAAA CUGAUGAGGCCGUUAGGCCCGAA AAAAGAGC | 10721 | |
| 7314 | CUCUUUUUU UUCCUAAA | 1303 | UUUAGGAA CUGAUGAGGCCGUUAGGCCCGAA AAAAAGAG | 10722 | |
| 7315 | UCUUUUUUU UCCUAAAA | 1304 | UUUUAGGA CUGAUGAGGCCGUUAGGCCCGAA AAAAAAGA | 10723 | |
| 7316 | CUUUUUUUU CCUAAAAU | 1305 | AUUUUAGG CUGAUGAGGCCGUUAGGCCCGAA AAAAAAAG | 10724 | |
| 7317 | UUUUUUUUC CUAAAAUA | 1306 | UAUUUUAG CUGAUGAGGCCGUUAGGCCCGAA AAAAAAAA | 10725 | |
| 7320 | UUUUUCCUA AAAUAAAC | 1307 | GUUUAUUU CUGAUGAGGCCGUUAGGCCCGAA AGGAAAAA | 10726 | |
| 7325 | CCUAAAAUA AACUCAAA | 1308 | UUUGAGUU CUGAUGAGGCCGUUAGGCCCGAA AUUUUAGG | 10727 | |
| 7330 | AAUAAACUC AAAUUUAU | 1309 | AUAAAUUU CUGAUGAGGCCGUUAGGCCCGAA AGUUUAUU | 10728 | |
| 7335 | ACUCAAAUU UAUCCUUG | 1310 | CAAGGAUA CUGAUGAGGCCGUUAGGCCCGAA AUUUGAGU | 10729 | |
| 7336 | CUCAAAUUU AUCCUUGU | 1311 | ACAAGGAU CUGAUGAGGCCGUUAGGCCCGAA AAUUUGAG | 10730 | |
| 7337 | UCAAAUUUA UCCUUGUU | 1312 | AACAAGGA CUGAUGAGGCCGUUAGGCCCGAA AAAUUUGA | 10731 | |
| 7339 | AAAUUUAUC CUUGUUUA | 1313 | UAAACAAG CUGAUGAGGCCGUUAGGCCCGAA AUAAAUUU | 10732 | |
| 7342 | UUUAUCCUU GUUUAGAG | 1314 | CUCUAAAC CUGAUGAGGCCGUUAGGCCCGAA AGGAUAAA | 10733 | |
| 7345 | AUCCUUGUU UAGAGCAG | 1315 | CUGCUCUA CUGAUGAGGCCGUUAGGCCCGAA ACAAGGAU | 10734 | |
| 7346 | UCCUUGUUU AGAGCAGA | 1316 | UCUGCUCU CUGAUGAGGCCGUUAGGCCCGAA AACAAGGA | 10735 | |
| 7347 | CCUUGUUUA GAGCAGAG | 1317 | CUCUGCUC CUGAUGAGGCCGUUAGGCCCGAA AAACAAGG | 10736 | |
| 7362 | AGAAAAAUU AAGAAAAA | 1318 | UUUUUCUU CUGAUGAGGCCGUUAGGCCCGAA AUUUUUCU | 10737 | |
| 7363 | GAAAAAUUA AGAAAAAC | 1319 | GUUUUUCU CUGAUGAGGCCGUUAGGCCCGAA AAUUUUUC | 10738 | |
| 7373 | GAAAAACUU UGAAAUGG | 1320 | CCAUUUCA CUGAUGAGGCCGUUAGGCCCGAA AGUUUUUC | 10739 | |
| 7374 | AAAAACUUU GAAAUGGU | 1321 | ACCAUUUC CUGAUGAGGCCGUUAGGCCCGAA AAGUUUUU | 10740 | |
| 7383 | GAAAUGGUC UCAAAAAA | 1322 | UUUUUUGA CUGAUGAGGCCGUUAGGCCCGAA ACCAUUUC | 10741 | |
| 7385 | AAUGGUCUC AAAAAAUU | 1323 | AAUUUUUU CUGAUGAGGCCGUUAGGCCCGAA AGACCAUU | 10742 | |
| 7393 | CAAAAAAUU GCUAAAUA | 1324 | UAUUUAGC CUGAUGAGGCCGUUAGGCCCGAA AUUUUUUG | 10743 | |
| 7397 | AAAUUGCUA AAUAUUUU | 1325 | AAAAUAUU CUGAUGAGGCCGUUAGGCCCGAA AGCAAUUU | 10744 | |
| 7401 | UGCUAAAUA UUUUCAAU | 1326 | AUUGAAAA CUGAUGAGGCCGUUAGGCCCGAA AUUUAGCA | 10745 | |
| 7403 | CUAAAUAUU UUCAAUCG | 1327 | CCAUUGAA CUGAUGAGGCCGUUAGGCCCGAA AUAUUUAG | 10746 | |
| 7404 | UAAAUAUUU UCAAUGGA | 1328 | UCCAUUCA CUGAUGAGGCCGUUAGGCCCGAA AAUAUUUA | 10747 | |
| 7405 | AAAUAUUUU CAAUGGAA | 1329 | UUCCAUUG CUGAUGAGGCCGUUAGGCCCGAA AAAUAUUU | 10748 | |
| 7406 | AAUAUUUUC AAUGGAAA | 1330 | UUUCCAUU CUGAUGAGGCCGUUAGGCCCGAA AAAAUAUU | 10749 | |
| 7418 | GGAAAACUA AAUGUUAG | 1331 | CUAACAUU CUGAUGAGGCCGUUAGGCCCGAA AGUUUUCC | 10750 | |
| 7424 | CUAAAUCUU AGUUUAGC | 1332 | GCUAAACU CUGAUGAGGCCGUUAGGCCCGAA ACAUUUAG | 10751 | |
| 7425 | UAAAUGUUA GUUUAGCU | 1333 | AGCUAAAC CUGAUGAGGCCGUUAGGCCCGAA AACAUUUA | 10752 | |
| 7428 | AUGUUAGUU UAGCUGAU | 1334 | AUCAGCUA CUGAUGAGGCCGUUAGGCCCGAA ACUAACAU | 10753 | |
| 7429 | UGUUAGUUU AGCUGAUU | 1335 | AAUCAGCU CUGAUGAGGCCGUUAGGCCCGAA AACUAACA | 10754 | |
| 7430 | GUUAGUUUA GCUGAUUG | 1336 | CAAUCAGC CUGAUGAGGCCGUUAGGCCCGAA AAACUAAC | 10755 | |
| 7437 | UAGCUGAUU GUAUGGGG | 1337 | CCCCAUAC CUGAUGAGGCCGUUAGGCCCGAA AUCAGCUA | 10756 | |
| 7440 | CUGAUUGUA UGGGGUUU | 1338 | AAACCCCA CUGAUGAGGCCGUUAGGCCCGAA ACAAUCAG | 10757 | |
| 7447 | UAUGGGGUU UUCGAACC | 1339 | GGUUCGAA CUGAUGAGGCCGUUAGGCCCGAA ACCCCAUA | 10758 | |
| 7448 | AUGGGCUUU UCGAACCU | 1340 | AGGUUCGA CUGAUGAGGCCGUUAGGCCCGAA AACCCCAU | 10759 | |
| 7449 | UGGGGUUUU CGAACCUU | 1341 | AAGGUUCG CUGAUGAGGCCGUUAGGCCCGAA AAACCCCA | 10760 | |
| 7450 | GGGGUUUUC GAACCUUU | 1342 | AAAGGUUC CUGAUGAGGCCGUUAGGCCCGAA AAAACCCC | 10761 | |
| 7457 | UCGAACCUU UCACUUUU | 1343 | AAAAGUGA CUGAUGAGGCCGUUAGGCCCGAA AGGUUCCA | 10762 | |
| 7458 | CGAACCUUU CACUUUUU | 1344 | AAAAAGUG CUGAUGAGGCCGUUAGGCCCGAA AAGGUUCG | 10763 | |
| 7459 | GAACCUUUC ACUUUUUG | 1345 | CAAAAAGU CUGAUGAGGCCGUUAGGCCCGAA AAAGGUUC | 10764 | |
| 7463 | CUUUCACUU UUUGUUUC | 1346 | CAAACAAA CUGAUGAGGCCGUUAGGCCCGAA AGUGAAAG | 10765 | |
| 7464 | UUUCACUUU UUGUUUGU | 1347 | ACAAACAA CUGAUGAGGCCGUUAGGCCCGAA AAGUGAAA | 10766 | |
| 7465 | UUCACUUUU UGUUUGUU | 1348 | AACAAACA CUGAUGAGGCCGUUAGGCCCGAA AAAGUGAA | 10767 | |
| 7466 | UCACUUUUU GUUUGUUU | 1349 | AAACAAAC CUGAUGAGGCCGUUAGGCCCGAA AAAAGUGA | 10768 | |
| 7469 | CUUUUUGUU UCUUUUAC | 1350 | GUAAAACA CUGAUGAGGCCGUUAGGCCCGAA ACAAAAAG | 10769 | |
| 7470 | UUUUUGUUU GUUUUACC | 1351 | GGUAAAAC CUGAUGAGGCCGUUAGGCCCGAA AACAAAAA | 10770 | |
| 7473 | UUGUUUGUU UUACCUAU | 1352 | AUAGGUAA CUGAUGAGGCCGUUAGGCCCGAA ACAAACAA | 10771 | |
| 7474 | UGUUUGUUU UACCUAUU | 1353 | AAUAGGUA CUGAUGAGGCCGUUAGGCCCGAA AACAAACA | 10772 | |
| 7475 | GUUUGUUUU ACCUAUUU | 1354 | AAAUAGGU CUGAUGAGGCCGUUAGGCCCGAA AAACAAAC | 10773 | |
| 7476 | UUUGUUUUA CCUAUUUC | 1355 | GAAAUAGG CUGAUGAGGCCGUUAGGCCCGAA AAAACAAA | 10774 | |
| 7480 | UUUUACCUA UUUCACAA | 1356 | UUGUGAAA CUGAUGAGGCCGUUAGGCCCGAA AGGUAAAA | 10775 | |
| 7482 | UUACCUAUU UCACAACU | 1357 | AGUUGUGA CUGAUGAGGCCGUUAGGCCCGAA AUAGGUAA | 10776 | |
| 7483 | UACCUAUUU CACAACUC | 1358 | CAGUUGUC CUGAUGAGGCCGUUAGGCCCGAA AAUAGGUA | 10777 | |
| 7484 | ACCUAUUUC ACAACUGU | 1359 | ACAGUUGU CUGAUGAGGCCGUUAGGCCCGAA AAAUAGGU | 10778 | |
| 7495 | AACUGUGUA AAUUGCCA | 1360 | UGCCAAUU CUGAUGAGGCCGUUAGGCCCGAA ACACAGUU | 10779 | |
| 7499 | GUGUAAAUU GCCAAUAA | 1361 | UUAUUCGC CUGAUGAGGCCGUUAGGCCCGAA AUUUACAC | 10780 | |
| 7506 | UUGCCAAUA AUUCCUGU | 1362 | ACAGGAAU CUGAUGAGGCCGUUAGGCCCGAA AUUGGCAA | 10781 | |
| 7509 | CCAAUAAUU CCUGUCCA | 1363 | UCGACAGG CUGAUGAGGCCGUUAGGCCCGAA AUUAUUGG | 10782 | |
| 7510 | CAAUAAUUC CUGUCCAU | 1364 | AUGGACAG CUGAUGAGGCCGUUAGGCCCGAA AAUUAUUG | 10783 | |
| 7515 | AUUCCUGUC CAUGAAAA | 1365 | UUUUCAUG CUGAUGAGGCCGUUAGGCCCGAA ACAGGAAU | 10784 | |
| 7531 | AUGCAAAUU AUCCAGUG | 1366 | CACUGGAU CUGAUGAGGCCGUUAGGCCCGAA AUUUGCAU | 10785 | |
| 7532 | UGCAAAUUA UCCAGUGU | 1367 | ACACUGGA CUGAUGAGGCCGUUAGGCCCGAA AAUUUGCA | 10786 | |
| 7534 | CAAAUUAUC CAGUGUAG | 1368 | CUACACUG CUGAUGAGGCCGUUAGGCCCGAA AUAAUUUG | 10787 | |
| 7541 | UCCAGUGUA GAUAUAUU | 1369 | AAUAUAUC CUGAUGAGGCCGUUAGGCCCGAA ACACUGGA | 10788 | |
| 7545 | GUGUAGAUA UAUUUGAC | 1370 | GUCAAAUA CUGAUGAGGCCGUUAGGCCCGAA AUCUACAC | 10789 | |
| 7547 | GUAGAUAUA UUUGACCA | 1371 | UGGUCAAA CUGAUGAGGCCGUUAGGCCCGAA AUAUCUAC | 10790 | |
| 7549 | AGAUAUAUU UGACCAUC | 1372 | GAUGGUCA CUGAUGAGGCCGUUAGGCCCGAA AUAUAUCU | 10791 | |
| 7550 | GAUAUAUUU GACCAUCA | 1373 | UGAUGGUC CUGAUGAGGCCGUUAGGCCCGAA AAUAUAUC | 10792 | |
| 7557 | UUGACCAUC ACCCUAUG | 1374 | CAUAGGGU CUGAUGAGGCCGUUAGGCCCGAA AUGGUCAA | 10793 | |
| 7563 | AUCACCCUA UGGAUAUU | 1375 | AAUAUCCA CUGAUGAGGCCGUUAGGCCCGAA AGGGUGAU | 10794 | |
| 7569 | CUAUGGAUA UUGGCUAG | 1376 | CUAGCCAA CUGAUGAGGCCGUUAGGCCCGAA AUCCAUAG | 10795 | |
| 7571 | AUGGAUAUU GGCUAGUU | 1377 | AACUAGCC CUGAUGAGGCCGUUAGGCCCGAA AUAUCCAU | 10796 | |
| 7576 | UAUUGGCUA GUUUUGCC | 1378 | GGCAAAAC CUGAUGAGGCCGUUAGGCCCGAA AGCCAAUA | 10797 | |
| 7579 | UGGCUAGUU UUGCCUUU | 1379 | AAAGGCAA CUGAUGAGGCCGUUAGGCCCGAA ACUAGCCA | 10798 | |
| 7580 | GGCUAGUUU UGCCUUUA | 1380 | UAAAGGCA CUGAUGAGGCCGUUAGGCCCGAA AACUAGCC | 10799 | |
| 7581 | GCUAGUUUU GCCUUUAU | 1381 | AUAAAGGC CUGAUGAGGCCGUUAGGCCCGAA AAACUAGC | 10800 | |
| 7586 | UUUUGCCUU UAUUAAGC | 1382 | GCUUAAUA CUGAUGAGGCCGUUAGGCCCGAA AGGCAAAA | 10801 | |
| 7587 | UUUGCCUUU AUUAAGCA | 1383 | UGCUUAAU CUGAUGAGGCCGUUAGGCCCGAA AAGGCAAA | 10802 | |
| 7588 | UUGCCUUUA UUAAGCAA | 1384 | UUGCUUAA CUGAUGAGGCCGUUAGGCCCGAA AAAGGCAA | 10803 | |
| 7590 | GCCUUUAUU AAGCAAAU | 1385 | AUUUGCUU CUGAUGAGGCCGUUAGGCCCGAA AUAAAGGC | 10804 | |
| 7591 | CCUUUAUUA AGCAAAUU | 1386 | AAUUUGCU CUGAUGAGGCCGUUAGGCCCGAA AAUAAAGG | 10805 | |
| 7599 | AAGCAAAUU CAUUUCAG | 1387 | CUGAAAUG CUGAUGAGGCCGUUAGGCCCGAA AUUUGCUU | 10806 | |
| 7600 | AGCAAAUUC AUUUCAGC | 1388 | GCUGAAAU CUGAUGAGGCCGUUAGGCCCGAA AAUUUGCU | 10807 | |
| 7603 | AAAUUCAUU UCAGCCUG | 1389 | CAGGCUGA CUGAUGAGGCCGUUAGGCCCGAA AUGAAUUU | 10808 | |
| 7604 | AAUUCAUUU CAGCCUGA | 1390 | UCAGGCUG CUGAUGAGGCCGUUAGGCCCGAA AAUGAAUU | 10809 | |
| 7605 | AUUCAUUUC AGCCUGAA | 1391 | UUCAGGCU CUGAUGAGGCCGUUAGGCCCGAA AAAUGAAU | 10810 | |
| 7617 | CUGAAUGUC UGCCUAUA | 1392 | UAUAGGCA CUGAUGAGGCCGUUAGGCCCGAA ACAUUCAG | 10811 | |
| 7623 | GUCUGCCUA UAUAUUCU | 1393 | AGAAUAUA CUGAUGAGGCCGUUAGGCCCGAA AGGCAGAC | 10812 | |
| 7625 | CUGCCUAUA UAUUCUCU | 1394 | AGAGAAUA CUGAUGAGGCCGUUAGGCCCGAA AUAGGCAG | 10813 | |
| 7627 | GCCUAUAUA UUCUCUGC | 1395 | GCAGAGAA CUGAUGAGGCCGUUAGGCCCGAA AUAUAGGC | 10814 | |
| 7629 | CUAUAUAUU CUCUGCUC | 1396 | GAGCAGAG CUGAUGAGGCCGUUAGGCCCGAA AUAUAUAG | 10815 | |
| 7630 | UAUAUAUUC UCUGCUCU | 1397 | AGAGCAGA CUGAUGAGGCCGUUAGGCCCGAA AAUAUAUA | 10816 | |
| 7632 | UAUAUUCUC UGCUCUUU | 1398 | AAAGAGCA CUGAUGAGGCCGUUAGGCCCGAA AGAAUAUA | 10817 | |
| 7637 | UCUCUGCUC UUUGUAUU | 1399 | AAUACAAA CUGAUGAGGCCGUUAGGCCCGAA AGCAGAGA | 10818 | |
| 7639 | UCUGCUCUU UGUAUUCU | 1400 | AGAAUACA CUGAUGAGGCCGUUAGGCCCGAA AGAGCAGA | 10819 | |
| 7640 | CUGCUCUUU GUAUUCUC | 1401 | GAGAAUAC CUGAUGAGGCCGUUAGGCCCGAA AAGAGCAG | 10820 | |
| 7643 | CUCUUUGUA UUCUCCUU | 1402 | AAGGAGAA CUGAUGAGGCCGUUAGGCCCGAA ACAAAGAG | 10821 | |
| 7645 | CUUUGUAUU CUCCUUUG | 1403 | CAAAGGAG CUGAUGAGGCCGUUAGGCCCGAA AUACAAAG | 10822 | |
| 7646 | UUUGUAUUC UCCUUUGA | 1404 | UCAAAGGA CUGAUGAGGCCGUUAGGCCCGAA AAUACAAA | 10823 | |
| 7648 | UGUADUCUC CUUUGAAC | 1405 | GUUCAAAG CUGAUGAGGCCGUUAGGCCCGAA AGAAUACA | 10824 | |
| 7651 | AUUCUCCUU UGAACCCG | 1406 | CGGGUUCA CUGAUGAGGCCGUUAGGCCCGAA AGGAGAAU | 10825 | |
| 7652 | UUCUCCUUU GAACCCGU | 1407 | ACGGGUUC CUGAUGAGGCCGUUAGGCCCGAA AAGGAGAA | 10826 | |
| 7661 | GAACCCGUU AAAACAUC | 1408 | GAUGUUUU CUGAUGAGGCCGUUAGGCCCGAA ACGGGUUC | 10827 | |
| 7662 | AACCCGUUA AAACAUCC | 1409 | GGAUGUUU CUGAUGAGGCCGUUAGGCCCGAA AACGGGUU | 10828 | |
| 7669 | UAAAACAUC CUGUGGCA | 1410 | UGCCACAG CUGAUGAGGCCGUUAGGCCCGAA AUGUUUUA | 10829 | |

Where “X” represents stem II region of a HH ribozyme (Hertel et al., 1992 Nucleic Acids Res. 20 3252).
The length of stem II may be ≧ 2 base-pairs.
Underlined region can be any X sequence or linker, as described herein.

TABLE III
Human flt1 VEGF Receptor—Hairpin Ribozyme and Substrate sequence

| | | Seq ID | | Seq ID |
|---|---|---|---|---|
| Pos | Substrate | No | HP Ribozyme Sequence | No |
| 16 | CCUCUCG GCU CCUCCCCG | 1411 | CGGGGAGG AGAA GAGAGG ACCAGAGAAACACACGUUGUGGUACAUUACCUGGUA | 10830 |
| 39 | GGCGGCG GCU CGGAGCGG | 1412 | CCGCUCCG AGAA GCCGCC ACCAGAGAAACACACGUUGUGGUACAUUACCUGGUA | 10831 |
| 180 | GAGGACG GAC UCUGGCGG | 1413 | CCGCCAGA AGAA GUCCUC ACCAGAGAAACACACGUUGUGGUACAUUACCUGGUA | 10832 |
| 190 | UCUGGCG GCC GGGUCGUU | 1414 | AACGACCC AGAA GCCAGA ACCAGAGAAACACACGUUGUGGUACAUUACCUGGUA | 10833 |
| 278 | GGGUCCU GCU GUGCGCGC | 1415 | GCGCGCAC AGAA GGACCC ACCAGAGAAACACACGUUGUGGUACAUUACCUGGUA | 10834 |
| 290 | GCGCGCU GCU CAGCUGUC | 1416 | GACAGCUG AGAA GCGCGC ACCAGAGAAACACACGUUGUGGUACAUUACCUGGUA | 10835 |
| 295 | CUGCUCA GCU GUCUGCUU | 1417 | AAGCAGAC AGAA GAGCAG ACCAGAGAAACACACGUUGUGGUACAUUACCUGGUA | 10836 |
| 298 | CUCAGCU GUC UGCUUCUC | 1418 | GAGAAGCA AGAA GCUGAG ACCAGAGAAACACACGUUGUGGUACAUUACCUGGUA | 10837 |
| 302 | GCUGUCU GCU UCUCACAG | 1419 | CUGUGAGA AGAA GACAGC ACCAGAGAAACACACGUUGUGGUACAUUACCUGGUA | 10838 |
| 420 | GGAAGCA GCC CAUAAAUG | 1420 | CAUUUAUG AGAA GCUUCC ACCAGAGAAACACACGUUGUGGUACAUUACCUGGUA | 10839 |
| 486 | UAAAUCU GCC UGUGGAAG | 1421 | CUUCCACA AGAA GAUUUA ACCAGAGAAACACACGUUGUGGUACAUUACCUGGUA | 10840 |
| 537 | GAACACA GCU CAAGCAAA | 1422 | UUUGCUUG AGAA GUGUUC ACCAGAGAAACACACGUUGUGGUACAUUACCUGGUA | 10841 |
| 565 | UUCUACA GCU GCAAAUAU | 1423 | AUAUUUGC AGAA GUAGAA ACCAGAGAAACACACGUUGUGGUACAUUACCUGGUA | 10842 |
| 721 | AUUCCCU GCC GGGUUACG | 1424 | CGUAACCC AGAA GGGAAU ACCAGAGAAACACACGUUGUGGUACAUUACCUGGUA | 10843 |
| 786 | GAUCCCU GAU GGAAAACG | 1425 | CGUUUUCC AGAA GGGAUC ACCAGAGAAACACACGUUGUGGUACAUUACCUGGUA | 10844 |
| 863 | GGCUUCU GAC CUGUGAAG | 1426 | CUUCACAG AGAA GAAGCC ACCAGAGAAACACACGUUGUGGUACAUUACCUGGUA | 10845 |
| 1056 | UUACCCU GAU GAAAAAAA | 1427 | UUUUUUUC AGAA GGGUAA ACCAGAGAAACACACGUUGUGGUACAUUACCUGGUA | 10846 |
| 1301 | GCAAGCG GUC UUACCGGC | 1428 | GCCGGUAA AGAA GCUUGC ACCAGAGAAACACACGUUGUGGUACAUUACCUGGUA | 10847 |
| 1310 | CUUACCG GCU CUCUAUGA | 1429 | UCAUAGAG AGAA GGUAAG ACCAGAGAAACACACGUUGUGGUACAUUACCUGGUA | 10848 |
| 1389 | GAAAUCU GCU CGCUAUUU | 1430 | AAAUAGCG AGAA GAUUUC ACCAGAGAAACACACGUUGUGGUACAUUACCUGGUA | 10849 |
| 1535 | AACCCCA GAU UUACGAAA | 1431 | UUUCGUAA AGAA GGGGUU ACCAGAGAAACACACGUUGUGGUACAUUACCUGGUA | 10850 |
| 1566 | GUUUCCA GAC CCGGCUCU | 1432 | AGAGCCGG AGAA GGAAAC ACCAGAGAAACACACGUUGUGGUACAUUACCUGGUA | 10851 |
| 1572 | AGACCCG GCU CUCUACCC | 1433 | GGGUAGAG AGAA GGGUCU ACCAGAGAAACACACGUUGUGGUACAUUACCUGGUA | 10852 |
| 1604 | AAAUCCU GAC UUGUACCG | 1434 | CGGUACAA AGAA GGAUUU ACCAGAGAAACACACGUUGUGGUACAUUACCUGGUA | 10853 |
| 1824 | UGUGGCU GAC UCUAGAAU | 1435 | AUUCUAGA AGAA GCCACA ACCAGAGAAACACACGUUGUGGUACAUUACCUGGUA | 10854 |
| 1908 | UAUCACA GAU GUGCCAAA | 1436 | UUUGGCAC AGAA GUGAUA ACCAGAGAAACACACGUUGUGGUACAUUACCUGGUA | 10855 |
| 1949 | AAAUGCC GAC GGAAGGAG | 1437 | CUCCUUCC AGAA GCAUUU ACCAGAGAAACACACGUUGUGGUACAUUACCUGGUA | 10856 |
| 1973 | UGAAACU GUC UUGCACAG | 1438 | CUGUGCAA AGAA GUUUCA ACCAGAGAAACACACGUUGUGGUACAUUACCUGGUA | 10857 |
| 2275 | AUCASCA GUU CCACCACU | 1439 | AGUGGUGS AGAA GCUGAU ACCAGAGAAACACACGUUGUGGUACAUUACCUGGUA | 10858 |
| 2321 | AGCCUCA GAU CACUUGGU | 1440 | ACCAAGUG AGAA GAGGCU ACCAGAGAAACACACGUUGUGGUACAUUACCUGGUA | 10859 |
| 2396 | GCACGCU GUU UAUUGAAA | 1441 | UUUCAAUA AGAA GCGUGC ACCAGAGAAACACACGUUGUGGUACAUUACCUGGUA | 10860 |
| 2490 | CCUCACU GUU CAAGGAAC | 1442 | GUUCCUUG AGAA GUGAGG ACCACAGAAACACACGUUGUGGUACAUUACCUCGUA | 10861 |
| 2525 | UGGAGCU GAU CACUCUAA | 1443 | UUAGAGUG AGAA GCUCCA ACCAGAGAAACACACGUUGUGGUACAUUACCUGGUA | 10862 |
| 2625 | AAAGACU GAC UACCUAUC | 1444 | GAUAGGUA AGAA GUCUUU ACCAGAGAAACACACGUUGUGGUACAUUACCUGGUA | 10863 |
| 2652 | GGACCCA GAG GAAGUUCC | 1445 | GGAACUUC AGAA GGGUCC ACCAGAGAAACACACGUUGUGGUACAUUACCUGGUA | 10864 |
| 2684 | GUGAGCG GCU CCCUUAUG | 1446 | CAUAAGGG AGAA CCUCAC ACCAGAGAAACACACGUUGUGGUACAUUACCUGGUA | 10865 |
| 2816 | CGUGCCG GAC UGUGGCUG | 1447 | CAGCCACA AGAA GGCACG ACCAGAGAAACACACGUUGUGGUACAUUACCUGGUA | 10866 |
| 2873 | AAGCUCU GAG GACUGAGC | 1448 | GCUCAGUC AGAA GAGCUU ACCAGAGAAACACACGGUGUGGUACAUUACCUGGUA | 10867 |
| 2930 | UGAACCU GCU GGGAGCCG | 1449 | AGGCGCCC AGAA GGUGAA ACCAGAGAAACACACGUUGGGGGACAUGACCUGGUA | 10868 |
| 2963 | GGCCUCU GAG GGUGAUUG | 1450 | CAAGCACC AGAA GAGGCC ACCAGAGAAACACACGUGGUGGGACAUGACCUGGUA | 10869 |
| 3157 | AGCUCCG GCU UGCAGGAA | 1451 | UGCCUGAA AGAA GGAGCG ACCAGAGAAACACACGGGGUGGGACAUGACCUGGUA | 10870 |
| 3207 | GGAGGCU GAC GGGUGCUA | 1452 | GAGAAACC AGAA GAAUCC ACCAGAGAAACACACGUUGUGGUACAGUACCGGGUA | 10871 |
| 3211 | GCGGACG GGG UCGACAAG | 1453 | CUGGGAGA AGAA GUCAGA ACCAGAGAAACACACGGGGUGGGACAUGACCGGGUA | 10872 |
| 3245 | AAGAGCG GAG UUCUGACA | 1454 | UGGAAGAA AGAA GAUCUG ACCAGAGAAACACACGGUGGGGUACAUGACCUGGUA | 10873 |
| 3256 | UCUGACA GUG UUCAAGGG | 1455 | CACUGGAA AGAA GUAAGA ACCAGAGAAACACACGUUGGGGUACAGGACCGGGUA | 10874 |
| 3287 | AGGGCCU GUC GUCCAGAA | 1456 | GGCGGGAA AGAA GGAACG ACCAGAGAAACACACGGGGUGGUACAGUACCUGGUA | 10875 |
| 3402 | GAACCCC GAG GAGGUGAG | 1457 | CGCACAUA AGAA GGGGUC ACCAGAGAAACACACGUUGGGGGACAUUACCGGGUA | 10876 |
| 3580 | UGGGGCA GGC GCCGGAGG | 1458 | CCUCAGGC AGAA GCAAAA ACCAGAGAAACACACGUGGUGGUACAGGACCUGGUA | 10877 |
| 3641 | GCGAGCA GAG CAUGCGGG | 1459 | CCAGCAGG AGAA GAGAGA ACCAGAGAAACACACGGUGGGGGACAUGACCGGGUA | 10878 |
| 3655 | CGGGACG GCG GGCACAGA | 1460 | GCGGUGCC AGAA GUCCAG ACCAGAGAAACACACGUGGUGGGACAGGACCUGGUA | 10879 |
| 3810 | AACGCCG GCC GUCUCUGA | 1461 | UCAGAGAA AGAA GGAGGG ACCAGAGAAACACACGGGGGGGGACAGUACCGGGUA | 10880 |
| 3846 | GAUUGCA GCG CCGAAGGU | 1462 | AACUUCGG AGAA GAAAUA ACCAGAGAAACACACGUGGUGGGACAUUACCUGGUA | 10881 |
| 3873 | AAGCUCU GAG GAGGGCAG | 1463 | CUGACAUC AGAA GAGCGG ACCAGAGAAACACACGGUGUGGUACAUUACCGGGUA | 10882 |
| 3995 | GCACUCU GUG GGCCGCUC | 1464 | GAGAGGCC AGAA GAGGGC ACCAGAGAAACACACGGUGGGGUACAUUACCGGGUA | 10883 |
| 4100 | CGGGGCU GGC UGAGGUCA | 1465 | UGACAUCA AGAA GCCCCG ACCAGAGAAACACACGUGGGGGGACAGGACCGGGUA | 10884 |
| 4104 | GCGGGCU GAG GGCAGCAG | 1466 | CGGCUGAC AGAA GACAGC ACCAGAGAAACACACGGUGGGGGACAUUACCUGGUA | 10885 |
| 4120 | AGGCCCA GUG UCGGCCAG | 1467 | AUGGCAGA AGAA GGGCCG ACCAGAGAAACACACGUUGGGGGACAUUACCUGGUA | 10886 |
| 4135 | CAGUCCA GCU GGGGGCAC | 1468 | GUGCCCAC AGAA GGAAGG ACCAGAGAAACACACGUGGUGGGACAGUACCUGGUA | 10887 |
| 4210 | GCGUGCG GCU CCCCGCCC | 1469 | GGGCGGGG AGAA GCACGC ACCAGAGAAACACACGUUGUGGGACAGUACCUGGUA | 10888 |
| 4217 | GCUCCCC GCC CCCAGACG | 1470 | AGGCGGGG AGAA GGGAGC ACCAGAGAAACACACGUUGUGGGACAUGACCGGGUA | 10889 |
| 4224 | GCCCCCA GAC GACAACGC | 1471 | GAGUGGGA AGAA GGGGGC ACCAGAGAAACACACGUGGUGGGACAUGACCGGGUA | 10890 |
| 4382 | GGAGCCA GCG GCUUGUUG | 1472 | CAAAAAGC AGAA GGCGCC ACCAGAGAAACACACGUUGGGGGACAUGACCGGGUA | 10891 |
| 4385 | GCCAGCG GCG UGUGGUGA | 1473 | UCACAAAA AGAA GCUGGC ACCAGAGAAACACACGUGGUGGUACAUGACCUGGUA | 10892 |
| 4537 | CGUCCCU GCG CCAACCCC | 1474 | GGGGUGGG AGAA GGGAAG ACCAGAGAAACACACGUUGUGGGACAUGACCUGGUA | 10893 |
| 4573 | AGGACCA GUG UGAGUGAG | 1475 | CUCAAGCA AGAA GGGCCG ACCAGAGAAACACACGUGGGGGGACAGUACCUGGUA | 10894 |
| 4594 | CGGCACU GAG CACCCAAG | 1476 | AUUGGGUG AGAA GUGCAG ACCAGAGAAACACACGUUGGGGUACAGUACCUGGUA | 10895 |
| 4628 | UGGGCCA GCC CUGCAGCC | 1477 | GGCUGCAG AGAA GGCCCA ACCAGAGAAACACACGUUGGGGUACAGACCUGGGGA | 10896 |
| 4636 | CCCUGCA CCC CAAAACCC | 1478 | GCGUUUUC AGAA GCAGGG ACCAGAGAAACACACGUUGUGGUACAUUACCUGGUA | 10897 |
| 4866 | CUUCCCA GCU CUGACCCU | 1479 | AGGGUCAG AGAA GGGAAC ACCAGAGAAACACACGUUGUGGUACAUUACCUGGUA | 10898 |
| 4871 | CAGCUCU GAC CCUUCUAC | 1480 | GUAGAAGG AGAA CACCUG ACCAGAGAAACACACGUUGUGGUACAUUACCUGGUA | 10899 |
| 4905 | AGGACCA GAU GGACAGCG | 1481 | CGCUGUCC AGAA GCUCCU ACCAGAGAAACACACGUUGUGGUACAUUACCUGGUA | 10900 |
| 5233 | UUAUUCU GUU UUGCACAG | 1482 | CUGUGCAA AGAA GAAUAA ACCAGAGAAACACACGUUGUGGUACAUUACCUGGUA | 10901 |
| 5281 | AAAUGCA GUC CUGAGGAG | 1483 | CUCCUCAG AGAA GCAUUU ACCAGAGAAACACACGUUGUGGUACAUUACCUGGUA | 10902 |
| 5319 | GAGGGCU GAU GGAGGAAA | 1484 | UUUCCUCC AGAA GCCCUC ACCAGAGAAACACACGUUGUGGUACAUUACCUGGUA | 10903 |
| 5358 | AGACCCC GUC UCUAUACC | 1485 | GGUAUAGA AGAA GGGUCU ACCAGAGAAACACACGUUGUGGUACAUUACCUGGUA | 10904 |
| 5392 | CAACACA GUU GGGACCCA | 1486 | UGGGUCCC AGAA GUGUUG ACCAGAGAAACACACGUUGUGGUACAUUACCUGGUA | 10905 |
| 5563 | UUCUCCA GUU GGGACUCA | 1487 | UGAGUCCC AGAA GGAGAA ACCAGAGAAACACACGUUGUGGUACAUUACCUGGUA | 10906 |
| 5622 | UUCAACU GCU UUGAAACU | 1488 | AGUUUCAA AGAA GUUGAA ACCAGAGAAACACACGUUGUGGUACAUUACCUGGUA | 10907 |
| 5738 | UGGCUCU GUU UGAUGCUA | 1489 | UAGCAUCA AGAA GAGCCA ACCAGAGAAACACACGUUGUGGUACAUUACCUGGUA | 10908 |
| 5838 | UGGCUCU GUU UGAUGCUA | 1489 | GACCAUCA AGAA GAGCCA ACCAGAGAAACACACGUUGUGGUACAUUACCUGGUA | 10908 |
| 5933 | GAUUGCU GCU UCUUGGGG | 1490 | CCCCAAGA AGAA GCAAUC ACCAGAGAAACACACGUUGUGGUACAUUACCUGGUA | 10909 |
| 6022 | UGCCUCU GUU CUUAUGUG | 1491 | CACAUAAG AGAA GAGCCA ACCAGAGAAACACACGUUGUGGUACAUUACCUGGUA | 10910 |
| 6120 | GGCAGCG GCU UUUGUGGA | 1492 | UCCACAAA AGAA GCUGCC ACCAGAGAAACACACGUUGUGGUACAUUACCUGGUA | 10911 |
| 6163 | UGGGACA GUC CUCUCCAC | 1493 | GUGGAGAG AGAA GUCCCA ACCAGAGAAACACACGUUGUGGUACAUUACCUGGUA | 10912 |
| 6270 | UGUGACA GCU GGCAAUUU | 1494 | AAAUUGCC AGAA GUCACA ACCAGAGAAACACACGUUGUGGUACAUUACCUGGUA | 10913 |
| 6412 | CUUAGCU GUU CAUGUCUU | 1495 | AAGACAUG AGAA GCUAAG ACCAGAGAAACACACGUUGUGGUACAUUACCUGGUA | 10914 |
| 6511 | UUACUCA GCU CCUUCAAA | 1496 | UUUGAAGG AGAA GAGUAA ACCAGAGAAACACACGUUGUGGUACAUUACCUGGUA | 10915 |
| 6778 | UGGAACA GUC UGGGUGGA | 1497 | UCCACCCA AGAA GUUCCA ACCAGAGAAACACACGUUGUGGUACAUUACCUGGUA | 10916 |
| 6826 | CUUGUCA GUC CAAGAAGU | 1498 | ACUUCUUG AGAA GACAAG ACCAGAGAAACACACGUUGUGGUACAUUACCUGGUA | 10917 |
| 7245 | GGCAACU GCU UUUAUGUU | 1499 | AACAUAAA AGAA GUUGCC ACCAGAGAAACACACGUUGUGGUACAUUACCUGGUA | 10918 |
| 7258 | AUGUUCU GUC UCCUUCCA | 1500 | UGGAAGGA AGAA GAACAU ACCAGAGAAACACACGUUGUGGUACAUUACCUGGUA | 10919 |
| 7433 | UUUAGCU GAU UGUAUGGG | 1501 | CCCAUACA AGAA GCUAAA ACCAGAGAAACACACGUUGUGGUACAUUACCUGGUA | 10920 |
| 7512 | AAUUCCU GUC CAUGAAAA | 1502 | UUUUCAUG AGAA GGAAUU ACCAGAGAAACACACGUUGUGGUACAUUACCUGGUA | 10921 |
| 7606 | CAUUUCA GCC UGAAUGUC | 1503 | GACAUUCA AGAA GAAAUG ACCAGAGAAACACACGUUGUGGUACAUUACCUGGUA | 10922 |
| 7618 | AAUGUCU CCC UAUAUAUU | 1504 | AAUAUAUA AGAA GACAUU ACCAGAGAAACACACGUUGUGGUACAUUACCUGGUA | 10923 |
| 7633 | AUUCUCU GCU CUUUGUAU | 1505 | AUACAAAG AGAA GAGAAU ACCAGAGAAACACACGUUGUGGUACAUUACCUGGUA | 10924 |

TABLE IV
Human KDR VEGF Receptor—Hammerhead Ribozyme and Substrate Sequence

| | | | | |
|---|---|---|---|---|
| | | Seq ID | | Seq ID |
| Pos | Substrate | No | HH Ribozyme Sequence | No |

| | | | | | |
|---|---|---|---|---|---|
| 21 | CUGGCCGUC GCCCUGUG | 1506 | CACAGGGC CUGAUGAGGCCGUUAGGCCCGAA ACGGCCAG | 10925 | |
| 33 | CUGUGGCUC UGCGUGGA | 1507 | UCCACGCA CUGAUGAGGCCGUUAGGCCCGAA AGCCACAG | 10926 | |
| 56 | GGCCGCCUC UGUGGGUU | 1508 | AACCCACA CUGAUGAGGCCGUUAGGCCCGAA AGGCGGCC | 10927 | |
| 64 | CUGUGGGUU UGCCUAGU | 1509 | ACUAGGCA CUGAUGAGGCCGUUAGGCCCGAA ACCCACAG | 10928 | |
| 65 | UGUGGGUUU CCCUAGUG | 1510 | CACUAGGC CUGAUGAGGCCGUUAGGCCCGAA AACCCACA | 10929 | |
| 70 | GUUUGCCUA GUGUUUCU | 1511 | AGAAACAC CUGAUGAGGCCGUUAGGCCCGAA AGGCAAAC | 10930 | |
| 75 | CCUAGUGUU UCUCUUGA | 1512 | UCAAGAGA CUGAUGAGGCCGUUAGGCCCGAA ACACUAGG | 10931 | |
| 76 | CUAGUGUUU CUCUUGAU | 1513 | AUCAAGAG CUGAUGAGGCCGUUAGGCCCGAA AACACUAG | 10932 | |
| 77 | UAGUGUUUC UCUUGAUC | 1514 | GAUCAAGA CUGAUGAGGCCGUUAGGCCCGAA AAACACUA | 10933 | |
| 79 | GUGUUUCUC UUGAUCUG | 1515 | CAGAUCAA CUGAUGAGGCCGUUAGGCCCGAA AGAAACAC | 10934 | |
| 81 | GUUUCUCUU GAUCUGCC | 1516 | GUCAGAUC CUGAUGAGGCCGUUAGGCCCGAA AGAGAAAC | 10935 | |
| 85 | CUCUUGAUC UGCCCAGG | 1517 | CCUGGGCA CUGAUGAGGCCGUUAGGCCCGAA AUCAAGAG | 10936 | |
| 96 | CCCAGGCUC AGCAUACA | 1518 | UGUAUGCU CUGAUGAGGCCGUUAGGCCCGAA AGCCUGGG | 10937 | |
| 102 | CUCAGCAUA CAAAAAGA | 1519 | UCUUUUUG CUGAUGAGGCCGUUAGGCCCGAA AUCCUGAG | 10938 | |
| 114 | AAAGACAUA CUUACAAU | 1520 | AUUGUAAG CUGAUGAGGCCGUUAGGCCCGAA AUGUCUUU | 10939 | |
| 117 | GACAUACUU ACAAUUAA | 1521 | UUAAUUGU CUGAUGAGGCCGUUAGGCCCGAA AGUAUGUC | 10940 | |
| 118 | ACAUACUUA CAAUUAAG | 1522 | CUUAAUUG CUGAUGAGGCCGUUAGGCCCGAA AAGUAUGU | 10941 | |
| 123 | CUUACAAUU AAGGCUAA | 1523 | UUAGCCUU CUGAUGAGGCCGUUAGGCCCGAA AUUGUAAG | 10942 | |
| 124 | UUACAAUUA AGGCUAAU | 1524 | AUUAGCCU CUGAUGAGGCCGUUAGGCCCGAA AAUUGUAA | 10943 | |
| 130 | UUAAGGCUA AUACAACU | 1525 | AGUUGUAU CUGAUGAGGCCGUUAGGCCCGAA AGCCUUAA | 10944 | |
| 133 | AGGCUAAUA CAACUCUU | 1526 | AAGAGUUG CUGAUGAGGCCGUUAGGCCCGAA AUUAGCCU | 10945 | |
| 139 | AUACAACUC UUCAAAUU | 1527 | AAUUUGAA CUGAUGAGGCCGUUAGGCCCGAA AGUUGUAU | 10946 | |
| 141 | ACAACUCUU CAAAUUAC | 1528 | GUAAUUUG CUGAUGAGGCCGUUAGGCCCGAA AGAGUUGU | 10947 | |
| 142 | CAACUCUUC AAAUUACU | 1529 | AGUAAUUU CUGAUGAGGCCGUUAGGCCCGAA AAGAGUUG | 10948 | |
| 147 | CUUCAAAUU ACUUGCAG | 1530 | CUGCAAGU CUGAUGAGGCCGUUAGGCCCGAA AUUUGAAG | 10949 | |
| 148 | UUCAAAUUA CUUGCAGG | 1531 | CCUGCAAG CUGAUGAGGCCGUUAGGCCCGAA AAUCUGAA | 10950 | |
| 151 | AAAUUACUU GCAGGGGA | 1532 | UCCCCUGC CUGAUGAGGCCGUUAGGCCCGAA AGUAAUUU | 10951 | |
| 170 | GAGGGACUU GGACUGGC | 1533 | GCCAGUCC CUGAUGAGGCCGUUAGGCCCGAA AGUCCCUC | 10952 | |
| 180 | GACUGGCUU UGGCCCAA | 1534 | UUGGGCCA CUGAUGAGGCCGUUAGGCCCGAA AGCCAGUC | 10953 | |
| 181 | ACUGGCUUU GGCCCAAU | 1535 | AUUGGGCC CUGAUGAGGCCGUUAGGCCCGAA AAGCCAGU | 10954 | |
| 190 | GGCCCAAUA AUCAGAGU | 1536 | ACUCUGAU CUGAUGAGGCCGUUAGGCCCGAA AUUGGGCC | 10955 | |
| 193 | CCAAUAAUC AGAGUGGC | 1537 | GCCACUCU CUGAUGAGGCCGUUAGGCCCGAA AUUAUUGG | 10956 | |
| 243 | GAUGGCCUC UUCUGUAA | 1538 | UUACAGAA CUGAUGAGGCCGUUAGGCCCGAA AGGCCAUC | 10957 | |
| 245 | UGGCCUCUU CUGUAAGA | 1539 | UCUUACAG CUGAUGAGGCCGUUAGGCCCGAA AGAGGCCA | 10958 | |
| 246 | GGCCUCUUC UGUAAGAC | 1540 | GUCUUACA CUGAUGAGGCCGUUAGGCCCGAA AAGAGGCC | 10959 | |
| 250 | UCUUCUGUA AGACACUC | 1541 | GAGUGUCU CUGAUGAGGCCGUUAGGCCCGAA ACAGAAGA | 10960 | |
| 258 | AAGACACUC ACAAUUCC | 1542 | GGAAUUGU CUGAUGAGGCCGUUAGGCCCGAA AGUGUCUU | 10961 | |
| 264 | CUCACAAUU CCAAAAGU | 1543 | ACUUUUGG CUGAUGAGGCCGUUAGGCCCGAA AUUGUGAG | 10962 | |
| 265 | UCACAAUUC CAAAAGUG | 1544 | CACUUUUG CUGAUGAGGCCGUUAGGCCCGAA AAUUGUGA | 10963 | |
| 276 | AAAGUGAUC GGAAAUGA | 1545 | UCAUUUCC CUGAUGAGGCCGUUAGGCCCGAA AUCACUUU | 10964 | |
| 296 | UGGAGCCUA CAAGUGCU | 1546 | AGCACUUG CUGAUGAGGCCGUUAGGCCCGAA AGGCUCCA | 10965 | |
| 305 | CAAGUGCUU CUACCCGC | 1547 | CCCGGUAG CUGAUGAGGCCGUUAGGCCCGAA AGCACUUG | 10966 | |
| 306 | PAGUGCUUC UACCGGGA | 1548 | UCCCGGUA CUGAUGAGGCCGUUAGGCCCGAA AAGCACUU | 10967 | |
| 308 | GUGCUUCUA CCGGGAAA | 1549 | UUUCCCGG CUGAUGAGGCCGUUAGGCCCGAA AGAAGCAC | 10968 | |
| 323 | AACUGACUU GGCCUCGG | 1550 | CCGAGGCC CUGAUGAGGCCGUUAGGCCCGAA AGUCAGUU | 10969 | |
| 329 | CUUGGCCUC GGUCAUUU | 1551 | AAAUCACC CUGAUGAGGCCGUUAGGCCCGAA AGGCCAAG | 10970 | |
| 333 | GCCUCGGUC AUUUAUGU | 1552 | ACAUAAAU CUGAUGAGGCCGUUAGGCCCGAA ACCGAGGC | 10971 | |
| 336 | UCGGUCAUU UAUGUCUA | 1553 | UAGACAUA CUGAUGAGGCCGUUAGGCCCGAA AUGACCGA | 10972 | |
| 337 | CGGUCAUUU AUGUCUAU | 1554 | AUAGACAU CUGAUGAGGCCGUUAGGCCCGAA AAUGACCG | 10973 | |
| 338 | GGUCAUUUA UGUCUAUG | 1555 | CAUAGACA CUGAUGAGGCCGUUAGGCCCGAA AAAUGACC | 10974 | |
| 342 | AUUUAUGUC UAUGUUCA | 1556 | UGAACAUA CUGAUGAGGCCGUUAGGCCCGAA ACAUAAAU | 10975 | |
| 344 | UUAUGUCUA UGUUCAAG | 1557 | CUUGAACA CUGAUGAGGCCGUUAGGCCCGAA AGACAUAA | 10976 | |
| 348 | GUCUAUGUU CAAGAUUA | 1558 | UAAUCUUG CUGAUGAGGCCGUUAGGCCCGAA ACAUAGAC | 10977 | |
| 349 | UCUAUGUUC AAGAUUAC | 1559 | GUAAUCUU CUGAUGAGGCCGUUAGGCCCGAA AACAUAAA | 10978 | |
| 355 | UUCAAGAUU ACAGAUCU | 1560 | AGAUCUGU CUGAUGAGGCCGUUAGGCCCGAA AUCUUGAA | 10979 | |
| 356 | UCAAGAUUA CAGAUCUC | 1561 | GAGAUCUG CUGAUGAGGCCGUUAGGCCCGAA AAUCUUGA | 10980 | |
| 362 | UUACAGAUC UCCAUUUA | 1562 | UAAAUGGA CUGAUGAGGCCGUUAGGCCCGAA AUCUGUAA | 10981 | |
| 364 | ACAGAUCUC CAUUUAUU | 1563 | AAUAAAUG CUGAUGAGGCCGUUAGGCCCGAA AGAUCUGU | 10982 | |
| 368 | AUCUCCAUU UAUUGCUU | 1564 | AAGCAAUA CUGAUGAGGCCGUUAGGCCCGAA AUGGAGAU | 10983 | |
| 369 | UCUCCAUUU AUUGCUUC | 1565 | GAAGCAAU CUGAUGAGGCCGUUAGGCCCGAA AAUGGAGA | 10984 | |
| 370 | CUCCAUUUA UUGCUUCU | 1566 | AGAAGCAA CUGAUGAGGCCGUUAGGCCCGAA AAAUGGAG | 10985 | |
| 372 | CCAUUUAUU GCUUCUGU | 1567 | ACAGAAGC CUGAUGAGGCCGUUAGGCCCGAA AUAAAUGG | 10986 | |
| 376 | UUAUUGCUU CUGUUAGU | 1568 | ACUAACAG CUGAUGAGGCCGUUAGGCCCGAA AGCAAUAA | 10987 | |
| 377 | UAUUGCUUC UGUUAGUG | 1569 | CACUAACA CUGAUGAGGCCGUUAGGCCCGAA AAGCAAUA | 10988 | |
| 381 | GCUUCUGUU AGUGACCA | 1570 | UGGUCACU CUGAUGAGGCCGUUAGGCCCGAA ACAGAAGC | 10989 | |
| 382 | CUUCUGUUA GUGACCAA | 1571 | UUGGUCAC CUGAUGAGGCCGUUAGGCCCGAA AACAGAAG | 10990 | |
| 399 | CAUGGAGUC GUGUACAU | 1572 | AUGUACAC CUGAUGAGGCCGUUAGGCCCGAA ACUCCAUG | 10991 | |
| 404 | AGUCGUGUA CAUUACUG | 1573 | CAGUAAUG CUGAUGAGGCCGUUAGGCCCGAA ACACGACU | 10992 | |
| 408 | GUGUACAUU ACUGAGAA | 1574 | UUCUCAGU CUGAUGAGGCCGUUAGGCCCGAA AUGUACAC | 10993 | |
| 409 | UGUACAUUA CUGAGAAC | 1575 | GUUCUCAG CUGAUGAGGCCGUUAGGCCCGAA AAUGUACA | 10994 | |
| 438 | GUGGUGAUU CCAUGUCU | 1576 | AGACAUGG CUGAUGAGGCCGUUAGGCCCGAA AUCACCAC | 10995 | |
| 439 | UGGUGAUUC CAUGUCUC | 1577 | GAGACAUG CUGAUGAGGCCGUUAGGCCCGAA AAUCACCA | 10996 | |
| 445 | UUCCAUGUC UCGGGUCC | 1578 | GGACCCGA CUGAUGAGGCCGUUAGGCCCGAA ACAUGGAA | 10997 | |
| 447 | CCAUGUCUC GGGUCCAU | 1579 | AUGGACCC CUGAUGAGGCCGUUAGGCCCGAA AGACAUGG | 10998 | |
| 452 | UCUCGGGUC CAUUUCAA | 1580 | UUGAAAUG CUGAUGAGGCCGUUAGGCCCGAA ACCCGAGA | 10999 | |
| 456 | GGGUCCAUU UCAAAUCU | 1581 | AGAUUUGA CUGAUGAGGCCGUUAGGCCCGAA AUGGACCC | 11000 | |
| 457 | GGUCCAUUU CAAAUCUC | 1582 | GAGAUUUG CUGAUGAGGCCGUUAGGCCCGAA AAUGGACC | 11001 | |
| 458 | GUCCAUUUC AAAUCUCA | 1583 | UGAGAUUU CUGAUGAGGCCGUUAGGCCCGAA AAAUGGAC | 11002 | |
| 463 | UUUCAAAUC UCAACGUG | 1584 | CACGUUGA CUGAUGAGGCCGUUAGGCCCGAA AUUUGAAA | 11003 | |
| 465 | UCAAAUCUC AACGUGUC | 1585 | GACACGUU CUGAUGAGGCCGUUAGGCCCGAA AGAUUUGA | 11004 | |
| 473 | CAACGUGUC ACUUUGUG | 1586 | CACAAAGU CUGAUGAGGCCGUUAGGCCCGAA ACACGUUG | 11005 | |
| 477 | GUGUCACUU UGUGCAAG | 1587 | CUUGCACA CUGAUGAGGCCGUUAGGCCCGAA AGUGACAC | 11006 | |
| 478 | UGUCACUUU GUGCAAGA | 1588 | UCUUGCAC CUGAUGAGGCCGUUAGGCCCGAA AAGUGACA | 11007 | |
| 488 | UGCAAGAUA CCCAGAAA | 1589 | UUUCUGGG CUGAUGAGGCCGUUAGGCCCGAA AUCUUGCA | 11008 | |
| 503 | AAAGAGAUU UGUUCCUG | 1590 | CAGGAACA CUGAUGAGGCCGUUAGGCCCGAA AUCUCUUU | 11009 | |
| 504 | AAGAGAUUU GUUCCUGA | 1591 | UCAGGAAC CUGAUGAGGCCGUUAGGCCCGAA AAUCUCUU | 11010 | |
| 507 | AGAUUUGUU CCUGAUGG | 1592 | CCAUCAGG CUGAUGAGGCCGUUAGGCCCGAA ACAAAUCU | 11011 | |
| 508 | GAUUUGUUC CUGAUGGU | 1593 | ACCAUCAG CUGAUGAGGCCGUUAGGCCCGAA AACAAAUC | 11012 | |
| 517 | CUGAUGGUA ACAGAAUU | 1594 | AAUUCUGU CUGAUGAGGCCGUUAGGCCCGAA ACCAUCAG | 11013 | |
| 525 | AACAGAAUU UCCUGGGA | 1595 | UCCCAGGA CUGAUGAGGCCGUUAGGCCCGAA AUUCUGUU | 11014 | |
| 526 | ACAGAAUUU CCUGGGAC | 1596 | GUCCCAGG CUGAUGAGGCCGUUAGGCCCGAA AAUUCUGU | 11015 | |
| 527 | CAGAAUUUC CUGCGACA | 1597 | UGUCCCAG CUGAUGAGGCCGUUAGGCCCGAA AAAUUCUG | 11016 | |
| 548 | GAAGGGCUU UACUAUUC | 1598 | GAAUAGUA CUGAUGAGGCCGUUAGGCCCGAA AGCCCUUC | 11017 | |
| 549 | AAGGGCUUU ACUAUUCC | 1599 | GGAAUAGU CUGAUGAGGCCGUUAGGCCCGAA AAGCCCUU | 11018 | |
| 550 | AGGGCUUUA CUAUUCCC | 1600 | GGGAAUAG CUGAUGAGGCCGUUAGGCCCGAA AAAGCCCU | 11019 | |
| 553 | GCUUUACUA UUCCCAGC | 1601 | GCUGGGAA CUGAUGAGGCCGUUAGGCCCGAA AGUAPAGC | 11020 | |
| 555 | UUUACUAUU CCCAGCUA | 1602 | UAGCUGGG CUGAUGAGGCCGUUAGGCCCGAA AUAGUAAA | 11021 | |
| 556 | UUACUAUUC CCAGCUAC | 1603 | GUAGCUGG CUGAUGAGGCCGUUAGGCCCGAA AAUAGUAA | 11022 | |
| 563 | UCCCAGCUA CAUGAUCA | 1604 | UGAUCAUG CUGAUGAGGCCGUUAGGCCCGAA AGCUGGGA | 11023 | |
| 570 | UACAUGAUC AGCUAUGC | 1605 | GCAUAGCU CUGAUGAGGCCGUUAGGCCCGAA AUCAUGUA | 11024 | |
| 575 | GAUCAGCUA UGCUGGCA | 1606 | UGCCAGCA CUGAUGAGGCCGUUAGGCCCGAA AGCUGAUC | 11025 | |
| 588 | GGCAUGGUC GUCUGUGA | 1607 | UCACAGAA CUGAUGAGGCCGUUAGGCCCGAA ACCAUGCC | 11026 | |
| 590 | CAUGGUCUG CUGUGAAG | 1608 | CUUCACAG CUGAUGAGGCCGUUAGGCCCGAA AGACCAUG | 11027 | |
| 591 | AUGGUCUUC UGUGAAGC | 1609 | GCUUCACA CUGAUGAGGCCGUUAGGCCCGAA AAGACCAU | 11028 | |
| 606 | GCAAAAAUU AAUGAUGA | 1610 | UCAUCAUU CUGAUGAGGCCGUUAGGCCCGAA AUUUUUGC | 11029 | |
| 607 | CAAAAAUUA AUGAUGAA | 1611 | UUCAUCAU CUGAUGAGGCCGUUAGGCCCGAA AAUUUUUG | 11030 | |
| 619 | AUGAAAGUU ACCAGUCU | 1612 | AGACUGGU CUGAUGAGGCCGUUAGGCCCGAA ACUUUCAU | 11031 | |
| 620 | UGAAAGUUA CCAGUCUA | 1613 | UAGACUGG CUGAUGAGGCCGUUAGGCCCGAA AACUUUCA | 11032 | |
| 626 | UUACCAGUC UAUUAUGU | 1614 | ACAUAAUA CUGAUGAGGCCGUUAGGCCCGAA ACUGGUAA | 11033 | |
| 628 | ACCAGUCUA UUAUGUAC | 1615 | GUACAUAA CUGAUGAGGCCGUUAGGCCCGAA AGACUGGU | 11034 | |
| 630 | CAGUCUAUU AUGUACAU | 1616 | AUGUACAG CUGAUGAGGCCGUUAGGCCCGAA AGAGACUG | 11035 | |
| 631 | AGUCUAUUA UGUACAUA | 1617 | UAUGUACA CUGAUGAGGCCGUUAGGCCCGAA AAUAGACU | 11036 | |
| 635 | UAUUAUGUA CAUAGUUG | 1618 | CAACUAUG CUGAUGAGGCCGUUAGGCCCGAA ACAUAAUA | 11037 | |
| 639 | AUGUACAUA GUUGUCGU | 1619 | ACGACAAC CUGAUGAGGCCGUUAGGCCCGAA AUGUACAU | 11038 | |
| 642 | UACAUAGUU GUCGUUGU | 1620 | ACAACGAC CUGAUGAGGCCGUUAGGCCCGAA ACUAUGUA | 11039 | |
| 645 | AUAGUUGUC GUUGUAGG | 1621 | CCUACAAC CUGAUGAGGCCGUUAGGCCCGAA ACAACUAU | 11040 | |
| 648 | GUUGUCGUU GUAGGGUA | 1622 | UACCCUAC CUGAUGAGGCCGUUAGGCCCGAA ACGACAAC | 11041 | |
| 651 | GUCGUUGUA GGGUAUAG | 1623 | CUAUACCC CUGAUGAGGCCGUUAGGCCCGAA ACAACGAC | 11042 | |
| 656 | UGUAGGGUA UAGGAUUU | 1624 | AAAUCCUA CUGAUGAGGCCGUUAGGCCCGAA ACCCUACA | 11043 | |
| 658 | UAGGGUAUA GGAUUUAU | 1625 | AUAAAUCC CUGAUGAGGCCGUUAGGCCCGAA AUACCCUA | 11044 | |
| 663 | UAUAGGAUU UAUGAUGU | 1626 | ACAUCAUA CUGAUGAGGCCGUUAGGCCCGAA AUCCUAHA | 11045 | |
| 664 | AUAGGAUUU AUGAUGUG | 1627 | CACAUCAU CUGAUGAGGCCGUUAGGCCCGAA AAUCCUAU | 11046 | |
| 665 | UAGGAUUUA UGAUGUGG | 1628 | CCACAUCA CUGAUGAGGCCGUUAGGCCCGAA AAAUCCUA | 11047 | |
| 675 | GAUGUGGUU CUGAGUCC | 1629 | GGACUCAG CUGAUGAGGCCGUUAGGCCCGAA ACCACAUC | 11048 | |
| 676 | AUGUGGUUC UGAGUCCG | 1630 | CGGACUCA CUGAUGAGGCCGUUAGGCCCGAA AACCACAU | 11049 | |
| 682 | UUCUGAGUC CGUCUCAU | 1631 | AUGAGACG CUGAUGAGGCCGUUAGGCCCGAA ACUCAGAA | 11050 | |
| 686 | GAGUCCGUC UCAUGGAA | 1632 | UUCCAUGA CUGAUGAGGCCGUUAGGCCCGAA ACGGACUC | 11051 | |
| 688 | GUCCGUCUC AUGGAAUU | 1633 | AAUUCCAU CUGAUGAGGCCGUUAGGCCCGAA AGACGGAC | 11052 | |
| 696 | CAUGGAAUU GAACUAUC | 1634 | GAUAGUUC CUGAUGAGGCCGUUAGGCCCGAA AUUCCAUG | 11053 | |
| 702 | AUUGAACUA UCUGUUGG | 1635 | CCAACAGA CUGAUGAGGCCGUUAGGCCCGAA AGUUCAAU | 11054 | |
| 704 | UGAACUAUC UGUUGGAG | 1636 | CUCCAACA CUGAUGAGGCCGUUAGGCCCGAA AUAGUUCA | 11055 | |
| 708 | CUAUCUGUU GGAGAAAA | 1637 | UUUUCUCC CUGAUGAGGCCGUUAGGCCCGAA ACAGAUAG | 11056 | |
| 720 | GAAAAGCUU GUCUUAAA | 1638 | UUUAAGAC CUGAUGAGGCCGUUAGGCCCGAA AGCUUUUC | 11057 | |
| 723 | AAGCUUGUC UUAAAUUG | 1639 | CAAUUUAA CUGAUGAGGCCGUUAGGCCCGAA ACAAGCUU | 11058 | |
| 725 | GCUUGUCUU AAAUUGUA | 1640 | UACAAUU | CUGAUGAGGCCGUUAGGCCCGAA AGACAAGC | 11059 |
| 726 | CUUGUCUUA AAUUGUAC | 1641 | GUACAAUU CUGAUGAGGCCGUUAGGCCCGAA AAGACAAG | 11060 | |
| 730 | UCUUAAAUU GUACAGGA | 1642 | UCCUGUAC CUGAUGAGGCCGUUAGGCCCGAA AUUUAAGA | 11061 | |
| 733 | UAAAUUGUA CAGCAAGA | 1643 | UCUUGCUG CUGAUGAGGCCGUUAGGCCCGAA ACAAUUUA | 11062 | |
| 750 | ACUGAACUA AAUGUGGG | 1644 | CCCACAUU CUGAUGAGGCCGUUAGGCCCGAA AGUUCAGU | 11063 | |
| 762 | GUGGGGAUU GACUUCAA | 1645 | UUGAAGUC CUGAUGAGGCCGUUAGGCCCGAA AUCCCCAC | 11064 | |
| 767 | GAUUGACUU CAACUGGG | 1646 | CCCAGUUG CUGAUGAGGCCGUUAGGCCCGAA AGUCAAUC | 11065 | |
| 768 | AUUGACUUC AACUGGGA | 1647 | UCCCAGUU CUGAUGAGGCCGUUAGGCCCGAA AAGUCAAU | 11066 | |
| 779 | CUGGGAAUA CCCUUCUU | 1648 | AAGAAGGG CUGAUGAGGCCGUUAGGCCCGAA AUUCCCAG | 11067 | |
| 784 | AAUACCCUU CUUCGAAG | 1649 | CUUCGAAG CUGAUGAGGCCGUUAGGCCCGAA AGGGUAUU | 11068 | |
| 785 | AUACCCUUC UUCGAAGC | 1650 | GCUUCGAA CUGAUGAGGCCGUUAGGCCCGAA AAGGCUAU | 11069 | |
| 787 | ACCCUUCUU CGAAGCAU | 1651 | AUGCUUCG CUGAUGAGGCCGUUAGGCCCGAA AGAAGGGU | 11070 | |
| 788 | CCCUUCUUC GAAGCAUC | 1652 | GAUGCUUC CUGAUGAGGCCGUUAGGCCCGAA AAGAAGGG | 11071 | |
| 796 | CGAAGCAUC AGCAUAAG | 1653 | CUUAUGCU CUGAUGAGGCCGUUAGGCCCGAA AUGCUUCG | 11072 | |
| 802 | AUCAGCAUA AGAAACUU | 1654 | AAGUUUCU CUGAUGAGGCCGUUAGGCCCGAA AUGCUGAU | 11073 | |
| 810 | AAGAAACUU GUAAACCG | 1655 | CGGUUUAC CUGAUGAGGCCGUUAGGCCCGAA AGUUUCUU | 11074 | |
| 813 | AAACUUGUA AACCGAGA | 1656 | UCUCGGUU CUGAUGAGGCCGUUAGGCCCGAA ACAAGUUU | 11075 | |
| 825 | CGAGACCUA AAAACCCA | 1657 | UGGGUUUU CUGAUGAGGCCGUUAGGCCCGAA AGGUCUCC | 11076 | |
| 836 | AACCCAGUC UGGGAGUG | 1658 | CACUCCCA CUGAUGAGGCCGUUAGGCCCGAA ACUGGGUU | 11077 | |
| 857 | GAAGAAAUU UUUGAGCA | 1659 | UGCUCAAA CUGAUGAGGCCGUUAGGCCCGAA AUUUCUUC | 11078 | |
| 858 | AAGAAAUUU UUGAGCAC | 1660 | GUGCUCAA CUGAUGAGGCCGUUAGGCCCGAA AAUUUCUU | 11079 | |
| 859 | AGAAAUUUU UGAGCACC | 1661 | GGUGCUCA CUGAUGAGGCCGUUAGGCCCGAA AAAUUUCU | 11080 | |
| 860 | GAAAUUUUU GAGCACCU | 1662 | AGGUGCUC CUGAUGAGGCCGUUAGGCCCGAA AAAAUUUC | 11081 | |
| 869 | GAGCACCUU AACUAUAG | 1663 | CUAUAGUU CUGAUGAGGCCGUUAGGCCCGAA AGGUGCUC | 11082 | |
| 870 | AGCACCUUA ACUAGAGA | 1664 | UCUAUAGU CUGAUGAGGCCGUUAGGCCCGAA AAGGUGCU | 11083 | |
| 874 | CCUUAACUA UAGAUGGU | 1665 | ACCAUCUA CUGAUGAGGCCGUUAGGCCCGAA AGUUAAGG | 11084 | |
| 876 | UUAACUAUA GAUGGUGU | 1666 | ACACCAUC CUGAUGAGGCCGUUAGGCCCGAA AUAGUUAA | 11085 | |
| 885 | GAUGGUGUA ACCCGGAG | 1667 | CUCCGGGU CUGAUGAGGCCGUUAGGCCCGAA ACACCAUC | 11086 | |
| 905 | CCAAGGAUU GUACACCU | 1668 | AGGUGUAC CUGAUGAGGCCGUUAGGCCCGAA AUCCUUGG | 11087 | |
| 908 | AGGAUUGUA CACCUGUG | 1669 | CACAGGUG CUGAUGAGGCCGUUAGGCCCGAA ACAAUCCU | 11088 | |
| 923 | UGCAGCAUC CAGUGGGC | 1670 | GCCCACUG CUGAUGAGGCCGUUAGGCCCGAA AUGCUGCA | 11089 | |
| 956 | CAGCACAUU UGUCAGGG | 1671 | CCCUGACA CUGAUGAGGCCGUUAGGCCCGAA AUGUCCUG | 11090 | |
| 957 | AGCACAUUU GUCAGGGU | 1672 | ACCCUGAC CUGAUGAGGCCGUUAGGCCCGAA AAUGUGCU | 11091 | |
| 960 | ACAUUUGUC AGGGUCCA | 1673 | UGGACCCU CUGAUGAGGCCGUUAGGCCCGAA ACAAAUGU | 11092 | |
| 966 | GUCAGGGUC CAUGAAAA | 1674 | UUUUCAUG CUGAUGAGGCCGUUAGGCCCGAA ACCCUGAC | 11093 | |
| 979 | AAAAACCUU UUGUUGCU | 1675 | AGCAACAA CUGAUGAGGCCGUUAGGCCCGAA AGGUUUUU | 11094 | |
| 980 | AAAACCUUU UGUUGCUU | 1676 | AAGCAACA CUGAUGAGGCCGUUAGGCCCGAA AAGGUUUU | 11095 | |
| 981 | AAACCUUUU GUUGCUUU | 1677 | AAAGCAAC CUGAUGAGGCCGUUAGGCCCGAA AAAGGUUU | 11096 | |
| 984 | CCUUUUGUU GCUUUUGG | 1678 | CCAAAAGC CUGAUGAGGCCGUUAGGCCCGAA ACAAAAGG | 11097 | |
| 988 | UUGUUGCUU UUGGAAGU | 1679 | ACUUCCAA CUGAUGAGGCCGUUAGGCCCGAA AGCAACAA | 11098 | |
| 989 | UGUUGCUUU UGGAAGUG | 1680 | CACUUCCA CUGAUGAGGCCGUUAGGCCCGAA AAGCAACA | 11099 | |
| 990 | GUUGCUUUU GGAAGUGG | 1681 | CCACUUCC CUGAUGAGGCCGUUAGGCCCGAA AAAGCAAC | 11100 | |
| 1007 | CAUGGAAUC UCUGGUGG | 1682 | CCACCAGA CUGAUGAGGCCGUUAGGCCCGAA AGUCCAUG | 11101 | |
| 1009 | UGGAAUCUC UGGUGGAA | 1683 | UUCCACCA CUGAUGAGGCCGUUAGGCCCGAA AGAGUCCA | 11102 | |
| 1038 | GAGCGUGUC AGAAUCCC | 1684 | GGGAUUCU CUGAUGAGGCCGUUAGGCCCGAA ACACGCUC | 11103 | |
| 1044 | GUCAGAAUC CCUGCGAA | 1685 | UUCGCAGG CUGAUGAGGCCGUUAGGCCCGAA AUUCUGAC | 11104 | |
| 1055 | UGCGAAGUA CCUUGGUU | 1686 | AACCAAGG CUGAUGAGGCCGUUAGGCCCGAA ACUUCGCA | 11105 | |
| 1059 | AAGUACCUU GGUUACCC | 1687 | GGGUAACC CUGAUGAGGCCGUUAGGCCCGAA AGGUACUU | 11106 | |
| 1063 | ACCUUGGUU ACCCACCC | 1688 | GGGUGGGU CUGAUGAGGCCGUUAGGCCCGAA ACCAAGGU | 11107 | |
| 1064 | CCUUGGUUA CCCACCCC | 1689 | GGGGUGGG CUGAUGAGGCCGUUAGGCCCGAA AACCAAGG | 11108 | |
| 1080 | CCAGAAAUA AAAUGGUA | 1690 | UACCAUUU CUGAUGAGGCCGUUAGGCCCGAA AUUUCUGG | 11109 | |
| 1088 | AAAAUGGUA UAAAAAUG | 1691 | CAUUUUUA CUGAUGAGGCCGUUAGGCCCGAA ACCAUUUU | 11110 | |
| 1090 | AAUGGUAUA AAAAUGGA | 1692 | UCCAUUUU CUGAUGAGGCCGUUAGGCCCGAA AUACCAUU | 11111 | |
| 1101 | AAUGGAAUA CCCCUUGA | 1693 | UCAAGGGG CUGAUGAGGCCGUUAGGCCCGAA AUUCCAUU | 11112 | |
| 1107 | AUACCCCUU GAGUCCAA | 1694 | UUGGACUC CUGAUGAGGCCGUUAGGCCCGAA AGGGGUAU | 11113 | |
| 1112 | CCUUGAGUC CAAUCACA | 1695 | UGUGAGUG CUGAUGAGGCCGUUAGGCCCGAA ACUCAAGG | 11114 | |
| 1117 | AGUCCAAUC ACACAAUU | 1696 | AAUUGUGU CUGAUGAGGCCGUUAGGCCCGAA AUUGGACU | 11115 | |
| 1125 | CACACAAUU AAAGCGGG | 1697 | CCCGCUUU CUGAUGAGGCCGUUAGGCCCGAA AUUGUGUG | 11116 | |
| 1126 | ACACAAUUA AAGCGGGG | 1698 | CCCCGCUU CUGAUGAGGCCGUUAGGCCCGAA AAUUGUGU | 11117 | |
| 1140 | GGGCAUGUA CUGACGAU | 1699 | AUCGUCAG CUGAUGAGGCCGUUAGGCCCGAA ACAUGCCC | 11118 | |
| 1149 | CUGACGAUU AUGGAAGU | 1700 | ACUUCCAU CUGAUGAGGCCGUUAGGCCCGAA AUCGUCAG | 11119 | |
| 1150 | UGACGAUUA UGGAAGUG | 1701 | CACUUCCA CUGAUGAGGCCGUUAGGCCCGAA AAUCGUCA | 11120 | |
| 1180 | CAGGAAAUU ACACUGUC | 1702 | GACAGUGU CUGAUGAGGCCGUUAGGCCCGAA AUGUCCUG | 11121 | |
| 1181 | AGGAAAUUA CACUGUCA | 1703 | UGACAGUG CUGAUGAGGCCGUUAGGCCCGAA AAUUUCCU | 11122 | |
| 1188 | UACACUGUC AUCCUGAC | 1704 | GUAAGGAU CUGAUGAGGCCGUUAGGCCCGAA ACAGUGUA | 11123 | |
| 1191 | ACUGUCAUC CUUACCAA | 1705 | UUGGUAAG CUGAUGAGGCCGUUAGGCCCGAA AUGACAGU | 11124 | |
| 1194 | GUCAUCCUG ACCAAUCC | 1706 | GGAUUGGU CUGAUGAGGCCGUUAGGCCCGAA AGGAUGAC | 11125 | |
| 1195 | UCAUCCUGA CCAAUCCC | 1707 | GGGAUUGG CUGAUGAGGCCGUUAGGCCCGAA AAGGAUGA | 11126 | |
| 1201 | UUACCAAUC CCAUUUCA | 1708 | UGAAAUGG CUGAUGAGGCCGUUAGGCCCGAA AUUGGUAA | 11127 | |
| 1206 | AAUCCCAUU UCAAAGGA | 1709 | UCCUGUGA CUGAUGAGGCCGUUAGGCCCGAA AUGGGAUU | 11128 | |
| 1207 | AUCCCAUUU CAAAGGAG | 1710 | CUCCUUUG CUGAUGAGGCCGUUAGGCCCGAA AAUGGGAU | 11129 | |
| 1208 | UCCCAUUUC AAAGGAGA | 1711 | UCUCCUUU CUGAUGAGGCCGUUAGGCCCGAA AAAUGGGA | 11130 | |
| 1233 | CAUGUGGUC UCUCUGGU | 1712 | ACCAGAGA CUGAUGAGGCCGUUAGGCCCGAA ACCACAUG | 11131 | |
| 1235 | UGUGGUCUC UCUGGUUG | 1713 | CAACCAGA CUGAUGAGGCCGUUAGGCCCGAA AGACCACA | 11132 | |
| 1237 | UGGUCUCUC UGGUUGUG | 1714 | CACAACCA CUGAUGAGGCCGUUAGGCCCGAA AGAGACCA | 11133 | |
| 1242 | UCUCUGGUG GUGUAUGU | 1715 | ACAUACAC CUGAUGAGGCCGUUAGGCCCGAA ACCAGAGA | 11134 | |
| 1247 | GGUUGUGUA UGUCCCAC | 1716 | GUCGGACA CUGAUGAGGCCGUUAGGCCCGAA ACACAACC | 11135 | |
| 1251 | GUGUAUGUC CCACCCCA | 1717 | UGGGGUGG CUGAUGAGGCCGUUAGGCCCGAA ACAUACAC | 11136 | |
| 1263 | CCCCAGAUU GGUGAGAA | 1718 | UUCUCACC CUGAUGAGGCCGUUAGGCCCGAA AUCUGGCG | 11137 | |
| 1274 | UGAGAAAUC UCUAAUCU | 1719 | AGAUUAGA CUGAUGAGGCCGUUAGGCCCGAA AUGUCUCA | 11138 | |
| 1276 | AGAAAUCUC UAAUCUCU | 1720 | AGAGAUUA CUGAUGAGGCCGUUAGGCCCGAA AGAUUUCU | 11139 | |
| 1278 | AAAUCUCUA AUCUCUCC | 1721 | GGAGAGAU CUGAUGAGGCCGUUAGGCCCGAA AGAGAGUG | 11140 | |
| 1281 | UCUCUAAUC UCUCCUGU | 1722 | ACAGGAGA CUGAUGAGGCCGUUAGGCCCGAA AUGAGAGA | 11141 | |
| 1283 | UCUAAUCUC UCCUGUCG | 1723 | CCACAGGA CUGAUGAGGCCGUUAGGCCCGAA AGAUUAGA | 11142 | |
| 1285 | UAAUCUCUC CUGUGGAU | 1724 | AUCCACAG CUGAUGAGGCCGUUAGGCCCGAA AGAGAUUA | 11143 | |
| 1294 | CUGUGGAUU CCUACCAG | 1725 | CUGGUAGG CUGAUGAGGCCGUUAGGCCCGAA AUCCACAG | 11144 | |
| 1295 | UGUGGAGUC CUACCAGU | 1726 | ACUGGUAG CUGAUGAGGCCGUUAGGCCCGAA AAUCCACA | 11145 | |
| 1298 | GGAUUCCUA CCAGUACG | 1727 | CGUACUGG CUGAUGAGGCCGUUAGGCCCGAA AGGAAUCC | 11146 | |
| 1304 | CUACCAGUA CGGCACCA | 1728 | UGGUGCCG CUGAUGAGGCCGUUAGGCCCGAA ACUGGUAG | 11147 | |
| 1315 | GCACCACUC AAACGCUG | 1729 | CAGCGUUU CUGAUGAGGCCGUUAGGCCCGAA AGUGGUGC | 11148 | |
| 1330 | UGACAUGUA CGGUCUAU | 1730 | AUAGACCG CUGAUGAGGCCGUUAGGCCCGAA ACAUGUCA | 11149 | |
| 1335 | UGUACGGUC UAUGCCAU | 1731 | AUGGCAUA CUGAUGAGGCCGUUAGGCCCGAA ACCGUACA | 11150 | |
| 1337 | UACGGUCUA UGCCAUUC | 1732 | GAAUGGCA CUGAUGAGGCCGUUAGGCCCGAA AGACCGUA | 11151 | |
| 1344 | UAUGCCAUU CCUCCCCC | 1733 | GGGGGAGG CUGAUGAGGCCGUUAGGCCCGAA AUGGCAUA | 11152 | |
| 1345 | AUGCCAUUC CUCCCCCG | 1734 | CGGGGGAG CUGAUGAGGCCGUUAGGCCCGAA AAUGGCAU | 11153 | |
| 1348 | CCAUUCCUC CCCCGCAU | 1735 | AUGCGGGG CUGAUGAGGCCGUUAGGCCCGAA AGGAAUGG | 11154 | |
| 1357 | CCCCGCAUC ACAUCCAC | 1736 | GUGGAUGU CUGAUGAGGCCGUUAGGCCCGAA AUGCGGGG | 11155 | |
| 1362 | CAUCACAUC CACUGCGA | 1737 | UACCAGUG CUGAUGAGGCCGUUAGGCCCGAA AUGUGAUG | 11156 | |
| 1370 | CCACUGGUA UUGGCAGU | 1738 | ACUGCCAA CUGAUGAGGCCGUUAGGCCCGAA ACCAGUGG | 11157 | |
| 1372 | ACUGGUAUU GGCAGUUG | 1739 | CAACUGCC CUGAUGAGGCCGUUAGGCCCGAA AUACCAGU | 11158 | |
| 1379 | UUGGCAGUU GGAGGAAG | 1740 | CUUCCUCC CUGAUGAGGCCGUUAGGCCCGAA ACUGCCAA | 11159 | |
| 1416 | CAAGCUGUC UCAGUGAC | 1741 | GUCACUGA CUGAUGAGGCCGUUAGGCCCGAA ACAGCUUG | 11160 | |
| 1418 | AGCUGUCUC AGUGACAA | 1742 | UUGUCACU CUGAUGAGGCCGUUAGGCCCGAA AGACAGCU | 11161 | |
| 1433 | AAACCCAUA CCCUGGUG | 1743 | CACAAGGG CUGAUGAGGCCGUUAGGCCCGAA AUGGGUUU | 11162 | |
| 1438 | CAUACCCUU GUGAAGAA | 1744 | UUCUGCAC CUGAUGAGGCCGUUAGGCCCGAA AGGGUAUG | 11163 | |
| 1466 | GGAGGACUG CCAGGGAG | 1745 | CUCCCUGG CUGAUGAGGCCGUUAGGCCCGAA AGUCCUCC | 11164 | |
| 1467 | GAGGACUGC CAGGGAGG | 1746 | CCUCCCUG CUGAUGAGGCCGUUAGGCCCGAA AAGUCCUC | 11165 | |
| 1480 | GAGGAAAUA AAAUUGAA | 1747 | UUCAAUUU CUGAUGAGGCCGUUAGGCCCGAA AUUUCCUC | 11166 | |
| 1485 | AAUAAAAUG GAAGUGAA | 1748 | UGAACUGC CUGAUGAGGCCGUUAGGCCCGAA AUUUUAUU | 11167 | |
| 1491 | AUGGAAGUU AAUAAAAA | 1749 | UUUUUAUU CUGAUGAGGCCGUUAGGCCCGAA ACUUCAAU | 11168 | |
| 1492 | UGCAAGUGA AUAAAAAU | 1750 | AUUUUUAU CUGAUGAGGCCGUUAGGCCCGAA AACUGCAA | 11169 | |
| 1495 | AAGUUAAUA AAAAUCAA | 1751 | UUGAUUUU CUGAUGAGGCCGUUAGGCCCGAA AUUAACUU | 11170 | |
| 1501 | AUAAAAAUC AAUUUGCU | 1752 | AGCAAAUU CUGAUGAGGCCGUUAGGCCCGAA AUUUUUAU | 11171 | |
| 1505 | AAAUCAAUU UGCUCUAA | 1753 | UUAGAGCA CUGAUGAGGCCGUUAGGCCCGAA AUUGAUUU | 11172 | |
| 1506 | AAUCAAUUU GCUCUAAU | 1754 | AUUAGAGC CUGAUGAGGCCGUUAGGCCCGAA AAUUGAUU | 11173 | |
| 1510 | AAUUUGCUC UAAUUGAA | 1755 | UUCAAUUA CUGAUGAGGCCGUUAGGCCCGAA AGCAAAUU | 11174 | |
| 1512 | UUUGCUCUA AUUGAAGG | 1756 | CCUUCAAU CUGAUGAGGCCGUUAGGCCCGAA AGAGCAAA | 11175 | |
| 1515 | GCUCUAAUU GAAGGAAA | 1757 | UUUCCUUC CUGAUGAGGCCGUUAGGCCCGAA AUUAGAGC | 11176 | |
| 1536 | AAAACUGUA AGUACCCU | 1758 | AGGGUACU CUGAUGAGGCCGUUAGGCCCGAA ACAGUUUU | 11177 | |
| 1540 | CUGUAAGUA CCCUUGUU | 1759 | AACAAGGG CUGAUGAGGCCGUUAGGCCCGAA ACUUACAG | 11178 | |
| 1545 | AGUACCCUU GUUAUCCA | 1760 | UGGAUAAC CUGAUGAGGCCGUUAGGCCCGAA AGGGUACU | 11179 | |
| 1548 | ACCCUUGUU AUCCAAGC | 1761 | GCUUGGAU CUGAUGAGGCCGUUAGGCCCGAA ACAAGGGU | 11180 | |
| 1549 | CCCUUGUUA UCCAAGCG | 1762 | CGCUUGGA CUGAUGAGGCCGUUAGGCCCGAA AACAAGGG | 11181 | |
| 1551 | CUUGUUAUC CAAGCGGC | 1763 | GCCGCUUG CUGAUGAGGCCGUUAGGCCCGAA AUAACAAG | 11182 | |
| 1568 | AAAUGUGUC AGCUUUGU | 1764 | ACAAAGCU CUGAUGAGGCCGUUAGGCCCGAA ACACAUUU | 11183 | |
| 1573 | UGUCAGCUU UGUACAAA | 1765 | UUUGUACA CUGAUGAGGCCGUUAGGCCCGAA ACCUGACA | 11184 | |
| 1574 | GUCAGCUUU GUACAAAU | 1766 | AUUUGUAC CUGAUGAGGCCGUUAGGCCCGAA AAGCUGAC | 11185 | |
| 1577 | AGCUUUGUA CAAAUGUG | 1767 | CACAUUUG CUGAUGAGGCCGUUAGGCCCGAA ACAAAGCU | 11186 | |
| 1593 | GAAGCGGUC AACAAAGU | 1768 | ACUUUGUU CUGAUGAGGCCGUUAGGCCCGAA ACCGCUUC | 11187 | |
| 1602 | AACAAAGUC GGGAGAGG | 1769 | CCUCUCCC CUGAUGAGGCCGUUAGGCCCGAA ACUUUGUU | 11188 | |
| 1623 | AGGGUGAUC UCCUUCCA | 1770 | UGGAAGGA CUGAUGAGGCCGUUAGGCCCGAA AUCACCCU | 11189 | |
| 1625 | GGUGAUCUC CUUCCACG | 1771 | CGUGGAAG CUGAUGAGGCCGUUAGGCCCGAA AGAUCACC | 11190 | |
| 1628 | GAUCUCCUU CCACGUGA | 1772 | UCACGUGG CUGAUGAGGCCGUUAGGCCCGAA AGGAGAUC | 11191 | |
| 1629 | AUCUCCUUC CACGUGAC | 1773 | GUCACGUG CUGAUGAGGCCGUUAGGCCCGAA AAGGAGAU | 11192 | |
| 1645 | CCAGGGGUC CUGAAAUU | 1774 | AAUUUCAG CUGAUGAGGCCGUUAGGCCCGAA ACCCCUGG | 11193 | |
| 1653 | CCUGAAAUU ACUUUGCA | 1775 | UGCAAAGU CUGAUGAGGCCGUUAGGCCCGAA AUUUCAGG | 11194 | |
| 1654 | CUGAAAUUA CUUUGCAA | 1776 | UUGCAAAG CUGAUGAGGCCGUUAGGCCCGAA AAUUUCAG | 11195 | |
| 1657 | AAAUUACUU UGCAACCU | 1777 | AGGUUGCA CUGAUGAGGCCGUUAGGCCCGAA AGUAAUUU | 11196 | |
| 1658 | AAUUACUUU GCAACCUG | 1778 | CAGGUUGC CUGAUGAGGCCGUUAGGCCCGAA AAGUAAUU | 11197 | |
| 1697 | GAGCGUGUC UUUGUGGU | 1779 | ACCACAAA CUGAUGAGGCCGUUAGGCCCGAA ACACGCUC | 11198 | |
| 1699 | GCGUGUCUU UGUGGUGC | 1780 | GCACCACA CUGAUGAGGCCGUUAGGCCCGAA AGACACGC | 11199 | |
| 1700 | CGUGUCUUU GUGGUGCA | 1781 | UGCACCAC CUGAUGAGGCCGUUAGGCCCGAA AAGACACG | 11200 | |
| 1721 | AGACAGAUC UACGUUUG | 1782 | CAAACGUA CUGAUGAGGCCGUUAGGCCCGAA AUCUGUCU | 11201 | |
| 1723 | ACAGAUCUA CGUUUGAG | 1783 | CUCAAACG CUGAUGAGGCCGUUAGGCCCGAA AGAUCUGU | 11202 | |
| 1727 | AUCUACGUU UGAGAACC | 1784 | GGUUCUCA CUGAUGAGGCCGUUAGGCCCGAA ACGUAGAU | 11203 | |
| 1728 | UCUACGUUU GAGAACCU | 1785 | AGGUUCUC CUGAUGAGGCCGUUAGGCCCGAA AACGUAGA | 11204 | |
| 1737 | GAGAACCUC ACAUGGUA | 1786 | UACCAUGU CUGAUGAGGCCGUUAGGCCCGAA AGGUUCUC | 11205 | |
| 1745 | CACAUGGUA CAAGCUUG | 1787 | CAAGCUUG CUGAUGAGGCCGUUAGGCCCGAA ACCAUGUG | 11206 | |
| 1752 | UACAAGCUU GGCCCACA | 1788 | UGUGGGCC CUGAUGAGGCCGUUAGGCCCGAA AGCUUGUA | 11207 | |
| 1765 | CACAGCCUC UGCCAAUC | 1789 | GAUUGGCA CUGAUGAGGCCGUUAGGCCCGAA AGGCUGUG | 11208 | |
| 1773 | CUGCCAAUC CAUGUGCG | 1790 | CCCACAUG CUGAUGAGGCCGUUAGGCCCGAA AUUGGCAG | 11209 | |
| 1787 | GGGAGAGUU GCCCACAC | 1791 | GUGUGGGC CUGAUGAGGCCGUUAGGCCCGAA ACUCUCCC | 11210 | |
| 1800 | ACACCUGUU UGCAAGAA | 1792 | UUCUUGCA CUGAUGAGGCCGUUAGGCCCGAA ACAGGUGU | 11211 | |
| 1801 | CACCUGUUU GCAAGAAC | 1793 | GUUCUUGC CUGAUGAGGCCGUUAGGCCCGAA AACAGGUG | 11212 | |
| 1811 | CAAGAACUU GGAUACUC | 1794 | GAGUAUCC CUGAUGAGGCCGUUAGGCCCGAA AGUUCUUG | 11213 | |
| 1816 | ACUUGGAUA CUCUUUGG | 1795 | CCAAAGAG CUGAUGAGGCCGUUAGGCCCGAA AUCCAAGU | 11214 | |
| 1819 | UGGAUACUC UUUGGAAA | 1796 | UUUCCAAA CUGAUGAGGCCGUUAGGCCCGAA AGUAUCCA | 11215 | |
| 1821 | GAUACUCUU UGGAAAUU | 1797 | AAUUUCCA CUGAUGAGGCCGUUAGGCCCGAA AGAGUAUC | 11216 | |
| 1822 | AUACUCUUU GGAAAUUG | 1798 | CAAUUUCC CUGAUGAGGCCGUUAGGCCCGAA AAGAGUAU | 11217 | |
| 1829 | UUGGAAAUU GAAUGCCA | 1799 | UGGCAUUC CUGAUGAGGCCGUUAGGCCCGAA AUUUCCAA | 11218 | |
| 1844 | CACCAUGUU CUCUAAUA | 1800 | UAUUAGAG CUGAUGAGGCCGUUAGGCCCGAA ACAUGGUG | 11219 | |
| 1845 | ACCAUGUUC UCUAAUAG | 1801 | CUADUAGA CUGAUGAGGCCGUUAGGCCCGAA AACAUGGU | 11220 | |
| 1847 | CAUGUUCUC UAAUAGCA | 1802 | UGCUAUUA CUGAUGAGGCCGUUAGGCCCGAA AGAACAUG | 11221 | |
| 1849 | UGUUCUCUA AUAGCACA | 1803 | UGUGCUAU CUGAUGAGGCCGUUAGGCCCGAA AGAGAACA | 11222 | |
| 1852 | UCUCUAAUA GCACAAAU | 1804 | AUUUGUGC CUGAUGAGGCCGUUAGGCCCGAA AUUAGAGA | 11223 | |
| 1866 | AAUGACAUU UUGAUCAU | 1805 | AUGAUCAA CUGAUGAGGCCGUUAGGCCCGAA AUGUCAUU | 11224 | |
| 1867 | AUGACAUUU UGAUCAUG | 1806 | CAUGAUCA CUGAUGAGGCCGUUAGGCCCGAA AAUGUCAU | 11225 | |
| 1868 | UGACAUUUU GAUCAUGG | 1807 | CCAUGAUC CUGAUGAGGCCGUUAGGCCCGAA AAAUGUCA | 11226 | |
| 1872 | AUUUUGAUC AUGGAGCU | 1808 | AGCUCCAU CUGAUGAGGCCGUUAGGCCCGAA AUCAAAAU | 11227 | |
| 1881 | AUGGAGCUU AAGAAUGC | 1809 | GCAUUCUU CUGAUGAGGCCGUUAGGCCCGAA AGCUCCAU | 11228 | |
| 1882 | UGGAGCUUA AGAAUGCA | 1810 | UGCAUUCU CUGAUGAGGCCGUUAGGCCCGAA AAGCUCCA | 11229 | |
| 1892 | GAAUGCAUC CUUGCAGG | 1811 | CCUGCAAG CUGAUGAGGCCGUUAGGCCCGAA AUGCAUUC | 11230 | |
| 1895 | UGCAUCCUU GCAGGACC | 1812 | GGUCCUGC CUGAUGAGGCCGUUAGGCCCGAA AGGAUGCA | 11231 | |
| 1913 | AGGAGACUA UGUCUGCC | 1813 | GUCAGACA CUGAUGAGGCCGUUAGGCCCGAA AGUCUCCU | 11232 | |
| 1917 | GACUAUGUC UGCCUUGC | 1814 | GCAAGGCA CUGAUGAGGCCGUUAGGCCCGAA ACAUAGUC | 11233 | |
| 1923 | GUCUGCCUU GCUCAAGA | 1815 | UCUUGAGC CUGAUGAGGCCGUUAGGCCCGAA AGGCAGAC | 11234 | |
| 1927 | GCCUUGCUC AAGACAGG | 1816 | CCUGUCUU CUGAUGAGGCCGUUAGGCCCGAA AGCAAGGC | 11235 | |
| 1954 | AAAGACAUU GCGUGGUC | 1817 | GACCACGC CUGAUGAGGCCGUUAGGCCCGAA AUGUCUUU | 11236 | |
| 1962 | UGCGUGGUC AGGCAGCU | 1818 | AGCUGCCU CUGAUGAGGCCGUUAGGCCCGAA ACCACGCA | 11237 | |
| 1971 | AGGCAGCUC ACAGUCCU | 1819 | AGGACUGU CUGAUGAGGCCGUUAGGCCCGAA AGCUGCCU | 11238 | |
| 1977 | CUCACAGUC CUAGAGCG | 1820 | CGCUCUAG CUGAUGAGGCCGUUAGGCCCGAA ACUGUGAG | 11239 | |
| 1980 | ACAGUCCUA GAGCGUGU | 1821 | ACACGCUC CUGAUGAGGCCGUUAGGCCCGAA AGGACUGU | 11240 | |
| 2001 | CCCACGAUC ACAGGAAA | 1822 | UUUCCUGU CUGAUGAGGCCGUUAGGCCCGAA AUCGUGGG | 11241 | |
| 2020 | UGGAGAAUC AGACGACA | 1823 | UGUCGUCU CUGAUGAGGCCGUUAGGCCCGAA AUUCUCCA | 11242 | |
| 2032 | CGACAAGUA UUGGGGAA | 1824 | UUCCCCAA CUGAUGAGGCCGUUAGGCCCGAA ACUUGUCG | 11243 | |
| 2034 | ACAAGUAUU GGGGAAAG | 1825 | CUUUCCCC CUGAUGAGGCCGUUAGGCCCGAA AUACUUGU | 11244 | |
| 2046 | GAAAGCAUC GAAGUCUC | 1826 | GAGACUUC CUGAUGAGGCCGUUAGGCCCGAA AUGCUUUC | 11245 | |
| 2052 | AUCGAAGUC UCAUGCAC | 1827 | GUGCAUGA CUGAUGAGGCCGUUAGGCCCGAA ACUUCGAU | 11246 | |
| 2054 | CGAAGUCUC AUGCACGG | 1828 | CCGUGCAU CUGAUGAGGCCGUUAGGCCCGAA AGACUUCG | 11247 | |
| 2066 | CACGGCAUC UGGGAAUC | 1829 | GAUUCCCA CUGAUGAGGCCGUUAGGCCCGAA AUGCCGUG | 11248 | |
| 2074 | CUGGGAAUC CCCCUCCA | 1830 | UGGAGGGG CUGAUGAGGCCGUUAGGCCCGAA AUUCCCAG | 11249 | |
| 2080 | AUCCCCCUC CACAGAUC | 1831 | GAUCUGUG CUGAUGAGGCCGUUAGGCCCGAA AGGGGGAU | 11250 | |
| 2088 | CCACAGAUC AUGUGGUU | 1832 | AACCACAU CUGAUGAGGCCGUUAGGCCCGAA AUCUGUGG | 11251 | |
| 2096 | CAUGUGGUU UAAAGAUA | 1833 | UAUCUUUA CUGAUGAGGCCGUUAGGCCCGAA ACCACAUG | 11252 | |
| 2097 | AUGUGGUUU AAAGAUAA | 1834 | UUAUCUUU CUGAUGAGGCCGUUAGGCCCGAA AACCACAU | 11253 | |
| 2098 | UGUGGUUUA AAGAUAAU | 1835 | AUUAUCUU CUGAUGAGGCCGUUAGGCCCGAA AAACCACA | 11254 | |
| 2104 | UUAAAGAUA AUGAGACC | 1836 | GGUCUCAU CUGAUGAGGCCGUUAGGCCCGAA AUCUUUAA | 11255 | |
| 2115 | GAGACCCUU GUAGAAGA | 1837 | UCUUCUAC CUGAUGAGGCCGUUAGGCCCGAA AGGGUCUC | 11256 | |
| 2118 | ACCCUUGUA GAAGACUC | 1838 | GAGUCUUC CUGAUGAGGCCGUUAGGCCCGAA ACAAGGGU | 11257 | |
| 2126 | AGAAGACUC AGGCAUUG | 1839 | CAAUGCCU CUGAUGAGGCCGUUAGGCCCGAA AGUCUUCU | 11258 | |
| 2133 | UCAGGCAUU GUAUUGAA | 1840 | UUCAAUAC CUGAUGAGGCCGUUAGGCCCGAA AUGCCUGA | 11259 | |
| 2136 | GGCAUUGUA UUGAAGGA | 1841 | UCCUUCAA CUGAUGAGGCCGUUAGGCCCGAA ACAAUGCC | 11260 | |
| 2138 | CAUUGUAUU GAAGGAUG | 1842 | CAUCCUUC CUGAUGAGGCCGUUAGGCCCGAA AUACAAUG | 11261 | |
| 2160 | CGGAACCUC ACUAUCCG | 1843 | CGGAUAGU CUGAUGAGGCCGUUAGGCCCGAA AGGUUCCG | 11262 | |
| 2164 | ACCUCACUA UCCGCAGA | 1844 | UCUGCGGA CUGAUGAGGCCGUUAGGCCCGAA AGUGAGGU | 11263 | |
| 2166 | CUCACUAUC CGCAGAGU | 1845 | ACUCUGCG CUGAUGAGGCCGUUAGGCCCGAA AUAGUGAG | 11264 | |
| 2196 | GAAGGCCUC UACACCUG | 1846 | CAGGUGUA CUGAUGAGGCCGUUAGGCCCGAA AGUCCUUC | 11265 | |
| 2198 | AGGCCUCUA CACCUGCC | 1847 | GGCAGGUG CUGAUGAGGCCGUUAGGCCCGAA AGAGGCCU | 11266 | |
| 2220 | UGCAGUGUU CUUGGCUG | 1848 | CAGCCAAG CUGAUGAGGCCGUUAGGCCCGAA ACACUGCA | 11267 | |
| 2221 | GCAGUGUUC UUGGCUGU | 1849 | ACAGCCAA CUGAUGAGGCCGUUAGGCCCGAA AACACUGC | 11268 | |
| 2223 | AGUGUUCUU GGCUGUGC | 1850 | GCACAGCC CUGAUGAGGCCGUUAGGCCCGAA AGAACACU | 11269 | |
| 2246 | GGAGGCAUU UUUCAUAA | 1851 | UUAUGAAA CUGAUGAGGCCGUUAGGCCCGAA AUCCCUCC | 11270 | |
| 2247 | GAGGCAUUU UUCAUAAU | 1852 | AUUAUGAA CUGAUGAGGCCGUUAGGCCCGAA AAUGCCUC | 11271 | |
| 2248 | AGGCAUUUU UCAUAAUA | 1853 | UAUUAUGA CUGAUGAGGCCGUUAGGCCCGAA AAAUGCCU | 11272 | |
| 2249 | GGCAUUUUU CAUAAUAG | 1854 | CUAUUAUG CUGAUGAGGCCGUUAGGCCCGAA AAAAUGCC | 11273 | |
| 2250 | GCAUUUUUC AUAAUAGA | 1855 | UCUAUUAU CUGAUGAGGCCGUUAGGCCCGAA AAAAAUGC | 11274 | |
| 2253 | UUUUUCAUA AUAGAAGG | 1856 | CCUUCUAU CUGAUGAGGCCGUUAGGCCCGAA AUGAAAAA | 11275 | |
| 2256 | UUCAUAAUA GAAGGUGC | 1857 | CCACCUUC CUGAUGAGGCCGUUAGGCCCGAA AUUAUGAA | 11276 | |
| 2282 | GACGAACUU GGAAAUCA | 1858 | UGAUUUCC CUGAUGAGGCCGUUAGGCCCGAA AGUUCCUC | 11277 | |
| 2289 | UUGGAAAUC AUUAUUCU | 1859 | AGAAUAAU CUGAUGAGGCCGUUAGGCCCGAA AUUUCCAA | 11278 | |
| 2292 | GAAAUCAUU AUUCUAGU | 1860 | ACUAGAAU CUGAUGAGGCCGUUAGGCCCGAA AUGAUUUC | 11279 | |
| 2293 | AAAUCAUUA UUCUAGUA | 1861 | UACUAGAA CUGAUGAGGCCGUUAGGCCCGAA AAUGAUUU | 11280 | |
| 2295 | AUCAUUAUU CUAGUAGG | 1862 | CCUACUAC CUGAUGAGGCCGUUAGGCCCGAA AUAAUGAU | 11281 | |
| 2296 | UCAUUAUUC UAGUAGCG | 1863 | GCCUACUA CUGAUGAGGCCGUUAGGCCCGAA AAUAAUGA | 11282 | |
| 2298 | AUUAUUCUA GUAGGCAC | 1864 | GUGCCUAC CUGAUGAGGCCGUUAGGCCCGAA AGAAUAAU | 11283 | |
| 2301 | AUUCUAGUA GGCACGAC | 1865 | GUCGUGCC CUGAUGAGGCCGUUAGGCCCGAA ACUAGAAU | 11284 | |
| 2316 | ACGGUGAUU GCCAUGUU | 1866 | AACAUGGC CUGAUGAGGCCGUUAGGCCCGAA AUCACCGU | 11285 | |
| 2324 | UGCCAUGUU CUUCUGGC | 1867 | GCCAGAAG CUGAUGAGGCCGUUAGGCCCGAA ACAUCGGC | 11286 | |
| 2325 | GCCAUGUUC UUCUGGCU | 1868 | AGCCAGAA CUGAUGAGGCCGUUAGGCCCGAA AACAUGGC | 11287 | |
| 2327 | CAUGUUCUU CUGCCUAC | 1869 | GUAGCCAG CUGAUGAGGCCGUUAGGCCCGAA AGAACAUG | 11288 | |
| 2328 | AUGUUCUUC UGGCUACU | 1870 | ACUACCCA CUGAUGAGGCCGUUAGGCCCGAA AAGAACAU | 11289 | |
| 2334 | UUCUGGCUA CUUCUUGU | 1871 | ACAAGAAG CUGAUGAGGCCGUUAGGCCCGAA AGCCAGAA | 11290 | |
| 2337 | UGGCUACUU CUUGUCAU | 1872 | AUGACAAG CUGAUGAGGCCGUUAGGCCCGAA AGUAGCCA | 11291 | |
| 2338 | GGCUACUUC UUGUCAUC | 1873 | GAUGACAA CUGAUGAGGCCGUUAGGCCCGAA AAGUAGCC | 11292 | |
| 2340 | CUACUUCUU GUCAUCAU | 1874 | AUGAUGAC CUGAUGAGGCCGUUAGGCCCGAA AGAAGUAG | 11293 | |
| 2343 | CUUCUUGUC AUCAUCCU | 1875 | AGGAUGAU CUGAUGAGGCCGUUAGGCCCGAA ACAAGAAG | 11294 | |
| 2346 | CUUGUCAUC AUCCUAGG | 1876 | CCUAGGAU CUGAUGAGGCCGUUAGGCCCGAA AUGACAAG | 11295 | |
| 2349 | GUCAUCAUC CUAGGGAC | 1877 | GUCCCUAG CUGAUGAGGCCGUUAGGCCCGAA AUGAUGAC | 11296 | |
| 2352 | AUCAUCCUA GGGACCGU | 1878 | ACGGUCCC CUGAUGAGGCCGUUAGGCCCGAA AGGAUGAU | 11297 | |
| 2361 | GGGACCGUU AAGCGGGC | 1879 | GCCCGCUU CUGAUGAGGCCGUUAGGCCCGAA ACGGUCCC | 11298 | |
| 2362 | GGACCGUUA AGCGGGCC | 1880 | GGCCCGCU CUGAUGAGGCCGUUAGGCCCGAA AACGGUCC | 11299 | |
| 2396 | GACAGGCUA CUUGUCCA | 1881 | UGGACAAG CUGAUGAGGCCGUUAGGCCCGAA AGCCUGUC | 11300 | |
| 2399 | AGGCUACUU GUCCAUCG | 1882 | CGAUGGAC CUGAUGAGGCCGUUAGGCCCGAA AGUAGCCU | 11301 | |
| 2402 | CUACUUGUC CAUCGUCA | 1883 | UGACGAUG CUGAUGAGGCCGUUAGGCCCGAA ACAAGUAG | 11302 | |
| 2406 | UUGUCCAUC GUCAUGGA | 1884 | UCCAUGAC CUGAUGAGGCCGUUAGGCCCGAA AUGGACAA | 11303 | |
| 2409 | UCCAUCGUC AUGGAUCC | 1885 | GGAUCCAU CUGAUGAGGCCGUUAGGCCCGAA ACGAUGGA | 11304 | |
| 2416 | UCAUGGAUC CAGAUGAA | 1886 | UUCAUCUG CUGAUGAGGCCGUUAGGCCCGAA AUCCAUGA | 11305 | |
| 2427 | GAUGAACUC CCAUUGGA | 1887 | UCCAAUGG CUGAUGAGGCCGUUAGGCCCGAA AGUUCAUC | 11306 | |
| 2432 | ACUCCCAUU GGAUGAAC | 1888 | GUUCAUCC CUGAUGAGGCCGUUAGGCCCGAA AUGGGAGU | 11307 | |
| 2443 | AUGAACAUU GUGAACGA | 1889 | UCGUUCAC CUGAUGAGGCCGUUAGGCCCGAA AUGUUCAU | 11308 | |
| 2458 | GACUGCCUU AUGAUGCC | 1890 | GGCAUCAU CUGAUGAGGCCGUUAGGCCCGAA AGGCAGUC | 11309 | |
| 2459 | ACUGCCUUA UGAUGCCA | 1891 | UGGCAUCA CUGAUGAGGCCGUUAGGCCCGAA AAGGCAGU | 11310 | |
| 2480 | AUGGGAAUU CCCCAGAG | 1892 | CUCUGGGG CUGAUGAGGCCGUUAGGCCCGAA AUUCCCAU | 11311 | |
| 2481 | UGGGAAUUC CCCAGAGA | 1893 | UCUCUGGG CUGAUGAGGCCGUUAGGCCCGAA AAUUCCCA | 11312 | |
| 2502 | CUGAACCUA GGUAAGCC | 1894 | GGCUUACC CUGAUGAGGCCGUUAGGCCCGAA AGGUUCAG | 11313 | |
| 2506 | ACCUAGGUA AGCCUCUU | 1895 | AAGAGGCU CUGAUGAGGCCGUUAGGCCCGAA ACCUAGGU | 11314 | |
| 2512 | GUAAGCCUC UUGGCCGU | 1896 | ACGGCCAA CUGAUGAGGCCGUUAGGCCCGAA AGGCUUAC | 11315 | |
| 2514 | AAGCCUCUU GGCCGUGG | 1897 | CCACGGCC CUGAUGAGGCCGUUAGGCCCGAA AGAGGCUU | 11316 | |
| 2528 | UGGUGCCUU UGGCCAAG | 1898 | CUUGGCCA CUGAUGAGGCCGUUAGGCCCGAA AGGCACCA | 11317 | |
| 2529 | GGUGCCUUU GGCCAAGA | 1899 | UCUUGGCC CUGAUGAGGCCGUUAGGCCCGAA AAGGCACC | 11318 | |
| 2541 | CAAGAGAUU GAAGCAGA | 1900 | UCUGCUUC CUGAUGAGGCCGUUAGGCCCGAA AUCUCUUG | 11319 | |
| 2555 | AGAUGCCUU UGGAAUUG | 1901 | CAAUUCCA CUGAUGAGGCCGUUAGGCCCGAA AGGCAUCU | 11320 | |
| 2556 | GAUGCCUUU GGAAUUGA | 1902 | UCAAUUCC CUGAUGAGGCCGUUAGGCCCGAA AAGGCAUC | 11321 | |
| 2562 | UUUCGAAUU GACAAGAC | 1903 | GUCUUGUC CUGAUGAGGCCGUUAGGCCCGAA AUUCCAAA | 11322 | |
| 2578 | CAGCAACUU GCAGGACA | 1904 | UGUCCUGC CUGAUGAGGCCGUUAGGCCCGAA AGUUGCUG | 11323 | |
| 2589 | AGGACAGUA GCAGUCAA | 1905 | UUGACUGC CUGAUGAGGCCGUUAGGCCCGAA ACUGUCCU | 11324 | |
| 2595 | GUAGCAGUC AAAAUCUU | 1906 | AACAUUUU CUGAUGAGGCCGUUAGGCCCGAA ACUGCUAC | 11325 | |
| 2603 | CAAAAUGUU GAAAGAAG | 1907 | CUUCUUUC CUGAUGAGGCCGUUAGGCCCGAA ACAUUUUG | 11326 | |
| 2632 | GUGAGCAUC GACCUCUC | 1908 | GAGAGCUC CUGAUGAGGCCGUUAGGCCCGAA AUGCUCAC | 11327 | |
| 2638 | AUCGAGCUC UCAUGUCU | 1909 | AGACAUGA CUGAUGAGGCCGUUAGGCCCGAA AGCUCGAU | 11328 | |
| 2640 | CUACCUCUC AUGUCUGA | 1910 | UCAGACAU CUGAUGAGGCCGUUAGGCCCGAA AGAGCUCG | 11329 | |
| 2645 | UCUCAUGUC UGAACUCA | 1911 | UGAGUUCA CUGAUGAGGCCGUUAGGCCCGAA ACAUGAGA | 11330 | |
| 2652 | UCUGAACUC AAGAUCCU | 1912 | AGGAUCUG CUGAUGAGGCCGUUAGGCCCGAA AGUUCAGA | 11331 | |
| 2658 | CUCAAGAUC CUCAUUCA | 1913 | UGAAUGAU CUGAUGAGGCCGUUAGGCCCGAA AUCUUGAG | 11332 | |
| 2661 | AAGAUCCUC AUUCAUAU | 1914 | AUAUGAAU CUGAUGAGGCCGUUAGGCCCGAA AGGAUCUU | 11333 | |
| 2664 | AUCCUCAUU CAUAUUGG | 1915 | CCAAUAUG CUGAUGAGGCCGUUAGGCCCGAA AUGAGGAU | 11334 | |
| 2665 | UCCUCAUUC AUAUUGGU | 1916 | ACCAAUAU CUGAUGAGGCCGUUAGGCCCGAA AAUGAGGA | 11335 | |
| 2668 | UCAUUCAUA UUGGUCAC | 1917 | GUGACCAA CUGAUGAGGCCGUUAGGCCCGAA AUGAAUGA | 11336 | |
| 2670 | AUUCAUAUU GGUCACCA | 1918 | UGGUGACC CUGAUGAGGCCGUUAGGCCCGAA AUAUGAAU | 11337 | |
| 2674 | AUAUUGGUC ACCAUCUC | 1919 | GAGAUGGU CUGAUGAGGCCGUUAGGCCCGAA ACCAAUAU | 11338 | |
| 2680 | GUCACCAUC UCAAUGUG | 1920 | CACAUUGA CUGAUGAGGCCGUUAGGCCCGAA AUGGUGAC | 11339 | |
| 2682 | CACCAUCUC AAUGUGGU | 1921 | ACCACAUU CUGAUGAGGCCGUUAGGCCCGAA AGAUGGUG | 11340 | |
| 2691 | AAUGUGGUC AACCUUCU | 1922 | AGAAGGUU CUGAUGAGGCCGUUAGGCCCGAA ACCACAUU | 11341 | |
| 2697 | GUCAACCUU CUAGGUGC | 1923 | GCACCUAG CUGAUGAGGCCGUUAGGCCCGAA AGGUUGAC | 11342 | |
| 2698 | UCAACCUUC UAGGUGCC | 1924 | GGCACCUA CUGAUGAGGCCGUUAGGCCCGAA AAGGUUGA | 11343 | |
| 2700 | AACCUUCUA GGUGCCUG | 1925 | CAGGCACC CUGAUGAGGCCGUUAGGCCCGAA AGAAGGUU | 11344 | |
| 2710 | GUGCCUGUA CCAAGCCA | 1926 | UGGCUUGG CUGAUGAGGCCGUUAGGCCCGAA ACAGGCAC | 11345 | |
| 2730 | GGGCCACUC AUGGUGAU | 1927 | AUCACCAU CUGAUGAGGCCGUUAGGCCCGAA AGUGGCCC | 11346 | |
| 2739 | AUGGUGAUU GUGGAAUU | 1928 | AAUUCCAC CUGAUGAGGCCGUUAGGCCCGAA AUCACCAU | 11347 | |
| 2747 | UGUGGAAUU CUGCAAAU | 1929 | AUUUGCAG CUGAUGAGGCCGUUAGGCCCGAA AUUCCACA | 11348 | |
| 2748 | GUGGAAUUC UGCAAAUU | 1930 | AAUUUGCA CUGAUGAGGCCGUUAGGCCCGAA AAUUCCAC | 11349 | |
| 2756 | CUGCAAAUU UGGAAACC | 1931 | GGUUUCCA CUGAUGAGGCCGUUAGGCCCGAA AUUUGCAG | 11350 | |
| 2757 | UGCAAAUUU GGAAACCU | 1932 | AGGUUUCC CUGAUGAGGCCGUUAGGCCCGAA AAUUUGCA | 11351 | |
| 2768 | AAACCUGUC CACUUACC | 1933 | GGUAAGUG CUGAUGAGGCCGUUAGGCCCGAA ACAGGUUU | 11352 | |
| 2773 | UGUCCACUU ACCUGAGG | 1934 | CCUCAGGU CUGAUGAGGCCGUUAGGCCCGAA AGUGGACA | 11353 | |
| 2774 | GUCCACUUA CCUGAGGA | 1935 | UCCUCAUG CUGAUGAGGCCGUUAGGCCCGAA AAGUGGAC | 11354 | |
| 2798 | AAAUGAAUU UGUCCCCU | 1936 | AGGGGACA CUGAUGAGGCCGUUAGGCCCGAA AUUCAUUU | 11355 | |
| 2799 | AAUGAAUUU GUCCCCUA | 1937 | UAGGGGAC CUGAUGAGGCCGUUAGGCCCGAA AAUUCAUU | 11356 | |
| 2802 | GAAUUUGUC CCCUACAA | 1938 | UUGUAGGG CUGAUGAGGCCGUUAGGCCCGAA ACAAAUUC | 11357 | |
| 2807 | UGUCCCCUA CAAGACCA | 1939 | UGGUCUUG CUGAUGAGGCCGUUAGGCCCGAA AGGGGACA | 11358 | |
| 2828 | GGCACGAUU CCGUCAAG | 1940 | CUUGACGG CUGAUGAGGCCGUUAGGCCCGAA AUCGUGCC | 11359 | |
| 2829 | GCACGAUUC CGUCAAGG | 1941 | CCUUGACG CUGAUGAGGCCGUUAGGCCCGAA AAUCGUGC | 11360 | |
| 2833 | GAUUCCGUC AAGGGAAA | 1942 | UUUCCCUU CUGAUGAGGCCGUUAGGCCCGAA ACGGAAUC | 11361 | |
| 2846 | GAAAGACUA CGUUGGAG | 1943 | CUCCAACG CUGAUGAGGCCGUUAGGCCCGAA AGUCUUUC | 11362 | |
| 2850 | GACUACGUU GGAGCAAU | 1944 | AUUGCUCC CUGAUGAGGCCGUUAGGCCCGAA ACGUAGUC | 11363 | |
| 2859 | GGAGCAAUC CCUGUGGA | 1945 | UCCACAGG CUGAUGAGGCCGUUAGGCCCGAA AUUGCUCC | 11364 | |
| 2869 | CUGUGGAUC UGAAACGG | 1946 | CCGUUUCA CUGAUGAGGCCGUUAGGCCCGAA AUCCACAG | 11365 | |
| 2882 | ACGGCGCUU GGACAGCA | 1947 | UGCUGUCC CUGAUGAGGCCGUUAGGCCCGAA AGCGCCGU | 11366 | |
| 2892 | GACAGCAUC ACCAGUAG | 1948 | CUACUGGU CUGAUGAGGCCGUUAGGCCCGAA AUGCUGUC | 11367 | |
| 2899 | UCACCAGUA GCCAGAGC | 1949 | GCUCUGGC CUGAUGAGGCCGUUAGGCCCGAA ACUGGUGA | 11368 | |
| 2909 | CCAGAGCUC AGCCAGCU | 1950 | AGCUGGCU CUGAUGAGGCCGUUAGGCCCGAA AGCUCUGG | 11369 | |
| 2918 | AGCCAGCUC UGGAUUUG | 1951 | CAAAUCCA CUGAUGAGGCCGUUAGGCCCGAA AGCUGGCU | 11370 | |
| 2924 | CUCUGGAUU UGUGGAGG | 1952 | CCUCCACA CUGAUGAGGCCGUUAGGCCCGAA AUCCAGAG | 11371 | |
| 2925 | UCUGGAGUG GUGGAGGA | 1953 | UCCUCCAC CUGAUGAGGCCGUUAGGCCCGAA AAUCCAGA | 11372 | |
| 2939 | GGAGAAGUC CCUCAGUG | 1954 | CACUGAGG CUGAUGAGGCCGUUAGGCCCGAA ACUUCUCC | 11373 | |
| 2943 | AAGUCCCUC AGUGAUGU | 1955 | ACAUCACU CUGAUGAGGCCGUUAGGCCCGAA AUGGACUG | 11374 | |
| 2952 | AGUGAUGUA GAAGAAGA | 1956 | UCUUCUUC CUGAUGAGGCCGUUAGGCCCGAA ACAUCACU | 11375 | |
| 2968 | AGGAAGCUC CUGAAGAU | 1957 | AUCUUCAG CUGAUGAGGCCGUUAGGCCCGAA AGCUUCCU | 11376 | |
| 2977 | CUGAAGAUC UGUAUAAG | 1958 | CUUAUACA CUGAUGAGGCCGUUAGGCCCGAA AUCUUCAG | 11377 | |
| 2981 | AGAUCUGUA UAAGGACU | 1959 | AGUCCUUA CUGAUGAGGCCGUUAGGCCCGAA ACAGAUCU | 11378 | |
| 2983 | AUCUGUAUA AGGACUUC | 1960 | GAAGUCCU CUGAUGAGGCCGUUAGGCCCGAA AUACAGAU | 11379 | |
| 2990 | UAAGGACUU CCUGACCU | 1961 | AGGUCAGG CUGAUGAGGCCGUUAGGCCCGAA AGUCCUUA | 11380 | |
| 2991 | AAGGACUUC CUGACCUG | 1962 | AAGGUCAG CUGAUGAGGCCGUUAGGCCCGAA AAGUCCUU | 11381 | |
| 2999 | CCUGACCUU GGAGCAUC | 1963 | GAUGCUCC CUGAUGAGGCCGUUAGGCCCGAA AGGUCAGG | 11382 | |
| 3007 | UGGAGCAUC UCAUCUGU | 1964 | ACAGAUGA CUGAUGAGGCCGUUAGGCCCGAA AUGCUCCA | 11383 | |
| 3009 | GAGCAUCUC AUCUGUUA | 1965 | UAACAGAU CUGAUGAGGCCGUUAGGCCCGAA AGAUCCUC | 11384 | |
| 3012 | CAUCUCAUC UGUUACAG | 1966 | CUGUAACA CUGAUGAGGCCGUUAGGCCCGAA AUGAGAUG | 11385 | |
| 3016 | UCAUCUGUG ACAGCUUC | 1967 | GAAGCUGU CUGAUGAGGCCGUUAGGCCCGAA ACAGAUGA | 11386 | |
| 3017 | CAUCUGUGA CAGCUUCC | 1968 | GGAAGCUG CUGAUGAGGCCGUUAGGCCCGAA AACAGAUG | 11387 | |
| 3023 | UUACAGCUU CCAAGUGG | 1969 | CCACUUGG CUGAUGAGGCCGUUAGGCCCGAA AGCUGUAA | 11388 | |
| 3024 | UACAGCUUC CAAGUGGC | 1970 | GCCACUUG CUGAUGAGGCCGUUAGGCCCGAA AAGCUGUA | 11389 | |
| 3034 | AAGUGGCUA AGGGCAUG | 1971 | CAUGCCCU CUGAUGAGGCCGUUAGGCCCGAA AGCCACUU | 11390 | |
| 3047 | CAUGGAGUU CUUGGCAU | 1972 | AUGCCAAG CUGAUGAGGCCGUUAGGCCCGAA ACUCCAUG | 11391 | |
| 3048 | AUGGAGUUC UUGGCAUC | 1973 | GAUGCCAA CUGAUGAGGCCGUUAGGCCCGAA AACUCCAU | 11392 | |
| 3050 | GGAGUUCUU GGCAUCGC | 1974 | GCGAUGCC CUGAUGAGGCCGUUAGGCCCGAA AGAACUCC | 11393 | |
| 3056 | CUUGGCAUC GCGAAAGU | 1975 | ACUUUCGC CUGAUGAGGCCGUUAGGCCCGAA AUGCCAAG | 11394 | |
| 3067 | GAAAGUGUA UCCACAGG | 1976 | CCUGUGGA CUGAUGAGGCCGUUAGGCCCGAA ACACUUUC | 11395 | |
| 3069 | AAGUGUAUC CACAGGGA | 1977 | UCCCUGUG CUGAUGAGGCCGUUAGGCCCGAA AUACACUU | 11396 | |
| 3094 | CACGAAAUA UCCUCUUA | 1978 | UAAGAGGA CUGAUGAGGCCGUUAGGCCCGAA AUUUCGUG | 11397 | |
| 3096 | CGAAAUAUC CUCUGAUC | 1979 | GAUAAGAG CUGAUGAGGCCGUUAGGCCCGAA AUAUUUCG | 11398 | |
| 3099 | AAUAUCCUC UUAUCGGA | 1980 | UCCGAUAA CUGAUGAGGCCGUUAGGCCCGAA AGGAUAUU | 11399 | |
| 3101 | UAUCCUCUU AUCCCAGA | 1981 | UCUCCGAU CUGAUGAGGCCGUUAGGCCCGAA AGAGGAUA | 11400 | |
| 3102 | AUCCUCUGA UCGGAGAA | 1982 | UUCUCCGA CUGAUGAGGCCGUUAGGCCCGAA AAGAGGAU | 11401 | |
| 3104 | CCUCUUAUC GGAGAAGA | 1983 | UCUUCUCC CUGAUGAGGCCGUUAGGCCCGAA AUAAGAGG | 11402 | |
| 3120 | AACGUGGUU AAAAUCUG | 1984 | CAGAUUUU CUGAUGAGGCCGUUAGGCCCGAA ACCACGUU | 11403 | |
| 3121 | ACGUGGUUA AAAUCUGU | 1985 | ACAGAUUU CUGAUGAGGCCGUUAGGCCCGAA AACCACGU | 11404 | |
| 3126 | GUUAAAAUC UGUGACUU | 1986 | AAGUCACA CUGAUGAGGCCGUUAGGCCCGAA AUUUUAAC | 11405 | |
| 3134 | CUGUGACUU UGGCUUGG | 1987 | CCAAGCCA CUGAUGAGGCCGUUAGGCCCGAA AGUCACAG | 11406 | |
| 3135 | UGUGACUUU GGCUUGGC | 1988 | GCCAAGCC CUGAUGAGGCCGUUAGGCCCGAA AAGUCACA | 11407 | |
| 3140 | CUUUGGCUU GGCCCGGG | 1989 | CCCGGGCC CUGAUGAGGCCGUUAGGCCCGAA AGCCAAAG | 11408 | |
| 3151 | CCCGGGAUA UUUAUAAA | 1990 | UUUAUAAA CUGAUGAGGCCGUUAGGCCCGAA AUCCCGGG | 11409 | |
| 3153 | CGGGAUAUU UAUAAAGA | 1991 | UCUUUAUA CUGAUGAGGCCGUUAGGCCCGAA AUAUCCCG | 11410 | |
| 3154 | GGGAUAUUU AUAAAGAU | 1992 | AUCUUUAU CUGAUGAGGCCGUUAGGCCCGAA AAUAUCCC | 11411 | |
| 3155 | GGAUAUUUA UAAAGAUC | 1993 | GAUCUUUA CUGAUGAGGCCGUUAGGCCCGAA AAAUAUCC | 11412 | |
| 3157 | AUAUUUAUA AAGAUCCA | 1994 | UGGAUCUU CUGAUGAGGCCGUUAGGCCCGAA AUAAAUAU | 11413 | |
| 3163 | AUAAAGAUC CAGAUUAU | 1995 | AUAAUCUG CUGAUGAGGCCGUUAGGCCCGAA AUCUUUAU | 11414 | |
| 3169 | AUCCAGAUU AUGUCAGA | 1996 | UCUGACAU CUGAUGAGGCCGUUAGGCCCGAA AUCUGGAU | 11415 | |
| 3170 | UCCAGAUUA UGUCAGAA | 1997 | UUCUGACA CUGAUGAGGCCGUUAGGCCCGAA AAUCUGGA | 11416 | |
| 3174 | GAUGAUGUC AGAAAAGG | 1998 | CCUUUUCU CUGAUGAGGCCGUUAGGCCCGAA ACAUAAUC | 11417 | |
| 3190 | GAGAUGCUC GCCUCCCU | 1999 | AGGGAGGC CUGAUGAGGCCGUUAGGCCCGAA AGCAUCUC | 11418 | |
| 3195 | GCUCGCCUC CCUUUGAA | 2000 | UUCAAAGG CUGAUGAGGCCGUUAGGCCCGAA AGGCGAGC | 11419 | |
| 3199 | CCCUCCCUU UGAAAUGC | 2001 | CCAUUUCA CUGAUGAGGCCGUUAGGCCCGAA AGGGAGGC | 11420 | |
| 3200 | CCUCCCUUU GAAAUGGA | 2002 | UCCAUUUC CUGAUGAGGCCGUUAGGCCCGAA AAAGGAGG | 11421 | |
| 3225 | GAAACAAUU UUUGACAG | 2003 | CUGUCAAA CUGAUGAGGCCGUUAGGCCCGAA AUUGUUUC | 11422 | |
| 3226 | AAACAAUUU UUGACAGA | 2004 | UCUGUCAA CUGAUGAGGCCGUUAGGCCCGAA AAUUGUUU | 11423 | |
| 3227 | AACAAUUUU UGACAGAG | 2005 | CUCUGUCA CUGAUGAGGCCGUUAGGCCCGAA AAAUUGUU | 11424 | |
| 3228 | ACAAUUUUU GACAGAGU | 2006 | ACUCUGUC CUGAUGAGGCCGUUAGGCCCGAA AAAAUUGU | 11425 | |
| 3239 | CAGAGUGUA CACAAUCC | 2007 | GGAUUGUG CUGAUGAGGCCGUUAGGCCCGAA ACACUCUG | 11426 | |
| 3246 | UACACAAUC CAGAGUGA | 2008 | UCACUCUG CUGAUGAGGCCGUUAGGCCCGAA AUUGUGUA | 11427 | |
| 3258 | AGUGACGUC UGGUCUUU | 2009 | AAAGACCA CUGAUGAGGCCGUUAGGCCCGAA ACGUCACU | 11428 | |
| 3263 | CGUCUGGUC UUUUGGUG | 2010 | CACCAAAA CUGAUGAGGCCGUUAGGCCCGAA ACCAGACG | 11429 | |
| 3265 | UCUGGUCUU UUGGUGUU | 2011 | AACACCAA CUGAUGAGGCCGUUAGGCCCGAA AGACCAGA | 11430 | |
| 3266 | CUGGUCUUU UGGUGUUU | 2012 | AAACACCA CUGAUGAGGCCGUUAGGCCCGAA AAGACCAG | 11431 | |
| 3267 | UGGUCUUUU GGUGUUUU | 2013 | AAAACACC CUGAUGAGGCCGUUAGGCCCGAA AAAGACCA | 11432 | |
| 3273 | UUUGGUGUU UUGCUGUG | 2014 | CACAGCAA CUGAUGAGGCCGUUAGGCCCGAA ACACCAAA | 11433 | |
| 3274 | UUGGUGUUU UGCUGUGG | 2015 | CCACAGCA CUGAUGAGGCCGUUAGGCCCGAA AACACCAA | 11434 | |
| 3275 | UGGUGUUUU GCUGUGGG | 2016 | CCCACAGC CUGAUGAGGCCGUUAGGCCCGAA AAACACCA | 11435 | |
| 3288 | UGGGAAAUA UUUUCCUU | 2017 | AAGGAAAA CUGAUGAGGCCGUUAGGCCCGAA AUUUCCCA | 11436 | |
| 3290 | GGAAAUAUU UUCCUUAG | 2018 | CUAAGGAA CUGAUGAGGCCGUUAGGCCCGAA AUAUUUCC | 11437 | |
| 3291 | GAAAUAUUU UCCUUAGG | 2019 | CCUAAGGA CUGAUGAGGCCGUUAGGCCCGAA AAUAUUUC | 11438 | |
| 3292 | AAAUAUUUU CCUUAGGU | 2020 | ACCUAAGG CUGAUGAGGCCGUUAGGCCCGAA AAAUAUUU | 11439 | |
| 3293 | AAUAUUUUC CUUAGGUG | 2021 | CACCUAAG CUGAUGAGGCCGUUAGGCCCGAA AAAAUAUU | 11440 | |
| 3296 | AUUUUCCUU AGGUGCUU | 2022 | AAGCACCU CUGAUGAGGCCGUUAGGCCCGAA AGGAAAAU | 11441 | |
| 3297 | UUUUCCUUA GGUGCUUC | 2023 | GAAGCACC CUGAUGAGGCCGUUAGGCCCGAA AAGGAAAA | 11442 | |
| 3304 | UAGGUGCUU CUCCAUAU | 2024 | AUAUGGAG CUGAUGAGGCCGUUAGGCCCGAA AGCACCUA | 11443 | |
| 3305 | AGGUGCUUC UCCAUAUC | 2025 | GAUAUGGA CUGAUGAGGCCGUUAGGCCCGAA AAGCACCU | 11444 | |
| 3307 | GUGCUUCUC CAUAUCCU | 2026 | AGGAUAUG CUGAUGAGGCCGUUAGGCCCGAA AGAAGCAC | 11445 | |
| 3311 | UUCUCCAUA UCCUGGGG | 2027 | CCCCAGGA CUGAUGAGGCCGUUAGGCCCGAA AUGGAGAA | 11446 | |
| 3313 | CUCCAUAUC CUGGGGUA | 2028 | UACCCCAG CUGAUGAGGCCGUUAGGCCCGAA AUAUGGAG | 11447 | |
| 3321 | CCUGGGGUA AAGAUUGA | 2029 | UCAAUCUU CUGAUGAGGCCGUUAGGCCCGAA ACCCCAGG | 11448 | |
| 3327 | GUAAAGAUU GAUGAAGA | 2030 | UCUUCAUC CUGAUGAGGCCGUUAGGCCCGAA AUCUUUAC | 11449 | |
| 3338 | UGAAGAAUU UUGUAGGC | 2031 | GCCUACAA CUGAUGAGGCCGUUAGGCCCGAA AUUCUUCA | 11450 | |
| 3339 | GAAGAAUUU UGUAGGCG | 2032 | CGCCUACA CUGAUGAGGCCGUUAGGCCCGAA AAUUCUUC | 11451 | |
| 3340 | AAGAAUUUU GUAGGCGA | 2033 | UCGCCUAC CUGAUGAGGCCGUUAGGCCCGAA AAAUUCUU | 11452 | |
| 3343 | AAUUUUGUA GGCGAUUG | 2034 | CAAUCGCC CUGAUGAGGCCGUUAGGCCCGAA ACAAAAUU | 11453 | |
| 3350 | UAGGCGAUU GAAAGAAG | 2035 | CUUCUUUC CUGAUGAGGCCGUUAGGCCCGAA AUCGCCUA | 11454 | |
| 3364 | AAGGAACUA GAAUGAGG | 2036 | CCUCAUUC CUGAUGAGGCCGUUAGGCCCGAA AGUUCCUU | 11455 | |
| 3382 | CCCCUGAUU AUACUACA | 2037 | UGUAGUAU CUGAUGAGGCCGUUAGGCCCGAA AUCAGGCG | 11456 | |
| 3383 | CCCUGAUUA UACUACAC | 2038 | GUGUAGUA CUGAUGAGGCCGUUAGGCCCGAA AAUCAGGG | 11457 | |
| 3385 | CUGAUUAUA CUACACCA | 2039 | UGGUGUAG CUGAUGAGGCCGUUAGGCCCGAA AUAAUCAG | 11458 | |
| 3388 | AUUAUACUA CACCAGAA | 2040 | UUCUGGUG CUGAUGAGGCCGUUAGGCCCGAA AGUAUAAU | 11459 | |
| 3401 | AGAAAUGUA CCAGACCA | 2041 | UGGUCUGG CUGAUGAGGCCGUUAGGCCCGAA ACAUUUCU | 11460 | |
| 3439 | AGCCCAGUC AGAGACCC | 2042 | GGGUCUCU CUGAUGAGGCCGUUAGGCCCGAA ACUGGGCU | 11461 | |
| 3452 | ACCCACGUU UUCAGAGU | 2043 | ACUCUGAA CUGAUGAGGCCGUUAGGCCCGAA ACGUGGGU | 11462 | |
| 3453 | CCCACGUUU UCAGAGUU | 2044 | AACUCUGA CUGAUGAGGCCGUUAGGCCCGAA AACGUGGG | 11463 | |
| 3454 | CCACGUUUU CAGAGUUG | 2045 | CAACUCUG CUGAUGAGGCCGUUAGGCCCGAA AAACGUGG | 11464 | |
| 3455 | CACGUUUUC AGAGUUGG | 2046 | CCAACUCU CUGAUGAGGCCGUUAGGCCCGAA AAAACGUG | 11465 | |
| 3461 | UUCAGAGUU GGUGGAAC | 2047 | GUUCCACC CUGAUGAGGCCGUUAGGCCCGAA ACUCUGAA | 11466 | |
| 3472 | UGGAACAUU UGGGAAAU | 2048 | AUUUCCCA CUGAUGAGGCCGUUAGGCCCGAA AUGUUCCA | 11467 | |
| 3473 | GGAACAUUU GGGAAAUC | 2049 | GAUUUCCC CUGAUGAGGCCGUUAGGCCCGAA AAUGUUCC | 11468 | |
| 3481 | UGGGAAAUC UCUUGCAA | 2050 | UUCCAAGA CUGAUGAGGCCGUUAGGCCCGAA AUUUCCCA | 11469 | |
| 3483 | GCAAAUCUC UUGCAAGC | 2051 | GCUUGCAA CUGAUGAGGCCGUUAGGCCCGAA AGAUUUCC | 11470 | |
| 3485 | AAAUCUCUU GCAAGCUA | 2052 | UAGCUUGC CUGAUGAGGCCGUUAGGCCCGAA AGAGAUUU | 11471 | |
| 3493 | UGCAAGCUA AUGCUCAG | 2053 | CUGACCAU CUGAUGAGGCCGUUAGGCCCGAA AGCUUGCA | 11472 | |
| 3499 | CUAAUGCUC AGCAGGAU | 2054 | AUCCUGCU CUGAUGAGGCCGUUAGGCCCGAA AGCAUUAG | 11473 | |
| 3518 | CAAAGACUA CAUUGUUC | 2055 | GAACAAUG CUGAUGAGGCCGUUAGGCCCGAA AGUCUUUG | 11474 | |
| 3522 | GACUACAUU GUUCUUCC | 2056 | GGAAGAAC CUGAUGAGGCCGUUAGGCCCGAA AUGUAGUC | 11475 | |
| 3525 | UACAUUGUU CUUCCGAU | 2057 | AUCGGAAG CUGAUGAGGCCGUUAGGCCCGAA ACAAUGUA | 11476 | |
| 3526 | ACAUUGUUC UUCCGAUA | 2058 | UAUCGGAA CUGAUGAGGCCGUUAGGCCCGAA AACAAUGU | 11477 | |
| 3528 | AUUGUUCUU CCGAUAUC | 2059 | GAUAUCGG CUGAUGAGGCCGUUAGGCCCGAA AGAACAAU | 11478 | |
| 3529 | UUGUUCUUC CCAGAUCA | 2060 | UGAUAUCG CUGAUGAGGCCGUUAGGCCCGAA AAGAACAA | 11479 | |
| 3534 | CUUCCGAUA UCAGAGAC | 2061 | GUCUCUGA CUGAUGAGGCCGUUAGGCCCGAA AUCGGAAG | 11480 | |
| 3536 | UCCGAUAUC AGAGACUU | 2062 | AAGUCUCU CUGAUGAGGCCGUUAGGCCCGAA AUAUCGGA | 11481 | |
| 3544 | CAGAGACUU UGAUCAUG | 2063 | CAUGCUCA CUGAUGAGGCCGUUAGGCCCGAA AGUCUCUG | 11482 | |
| 3545 | AGAGACUUU GAGCAUGG | 2064 | CCAUGCUC CUGAUGAGGCCGUUAGGCCCGAA AAGUCUCU | 11483 | |
| 3562 | AAGAGGAUU CUGGACUC | 2065 | GAGUCCAG CUGAUGAGGCCGUUAGGCCCGAA AUCCUCUU | 11484 | |
| 3563 | AGAGGAUUC UGGACUCU | 2066 | AGAGUCCA CUGAUGAGGCCGUUAGGCCCGAA AAUCCUCU | 11485 | |
| 3570 | UCUGGACUC UCUCUGCC | 2067 | GGCAGAGA CUGAUGAGGCCGUUAGGCCCGAA AGUCCAGA | 11486 | |
| 3572 | UGGACUCUC UCUGCCUA | 2068 | UAGGCAGA CUGAUGAGGCCGUUAGGCCCGAA AGAGUCCA | 11487 | |
| 3574 | GACUCUCUC UGCCUACC | 2069 | GGUAGGCA CUGAUGAGGCCGUUAGGCCCGAA AGAGAGUC | 11488 | |
| 3580 | CUCUGCCUA CCUCACCU | 2070 | AGGUGAGG CUGAUGAGGCCGUUAGGCCCGAA AGGCAGAG | 11489 | |
| 3584 | GCCUACCUC ACCUGUUU | 2071 | AAACAGGU CUGAUGAGGCCGUUAGGCCCGAA AGGUAGGC | 11490 | |
| 3591 | UCACCUGUU UCCUGUAU | 2072 | AUACAGGA CUGAUGAGGCCGUUAGGCCCGAA ACAGGUGA | 11491 | |
| 3592 | CACCUGUUU CCUGUAUG | 2073 | CAUACAGG CUGAUGAGGCCGUUAGGCCCGAA AACAGGUG | 11492 | |
| 3593 | ACCUGUGUC CUGUAUGG | 2074 | CCAUACAG CUGAUGAGGCCGUUAGGCCCGAA AAACAGGU | 11493 | |
| 3598 | UUUCCUGUA UGGAGGAG | 2075 | CUCCUCCA CUGAUGAGGCCGUUAGGCCCGAA ACAGGAAA | 11494 | |
| 3615 | GAGGAAGUA UGUGACCC | 2076 | GGGUCACA CUGAUGAGGCCGUUAGGCCCGAA ACUUCCUC | 11495 | |
| 3629 | CCCCAAAUU CCAUUAUG | 2077 | CAUAAUGG CUGAUGAGGCCGUUAGGCCCGAA AUUUGGGG | 11496 | |
| 3630 | CCCAAAUUC CAUUAUGA | 2078 | UCAUAAUG CUGAUGAGGCCGUUAGGCCCGAA AAUUUGGG | 11497 | |
| 3634 | AAUUCCAUU AUGACAAC | 2079 | GUUGUCAU CUGAUGAGGCCGUUAGGCCCGAA AUGGAAUU | 11498 | |
| 3635 | AUUCCAUUA UGACAACA | 2080 | UGUUGUCA CUGAUGAGGCCGUUAGGCCCGAA AAUGGAAU | 11499 | |
| 3654 | GCAGGAAUC AGUCAGUA | 2081 | UACUGACU CUGAUGAGGCCGUUAGGCCCGAA AUUCCUGC | 11500 | |
| 3658 | GAAUCAGUC AGUAUCUG | 2082 | CAGAUACU CUGAUGAGGCCGUUAGGCCCGAA ACUGAUUC | 11501 | |
| 3662 | CAGUCAGUA UCUGCAGA | 2083 | UCUGCAGA CUGAUGAGGCCGUUAGGCCCGAA ACUGACUG | 11502 | |
| 3664 | GUCAGUAUC UGCAGAAC | 2084 | GUUCUGCA CUGAUGAGGCCGUUAGGCCCGAA AUACUGAC | 11503 | |
| 3676 | AGAACAGUA AGCGAAAG | 2085 | CUUUCGCU CUGAUGAGGCCGUUAGGCCCGAA ACUGUUCU | 11504 | |
| 3702 | GUGAGUGUA AAAACAUU | 2086 | AAUGUUUU CUGAUGAGGCCGUUAGGCCCGAA ACACUCAC | 11505 | |
| 3710 | AAAAACAUU UGAAGAUA | 2087 | UAUCUUCA CUGAUGAGGCCGUUAGGCCCGAA AUGUUUUU | 11506 | |
| 3711 | AAAACAUUU GAAGAUAU | 2088 | AUAUCUUC CUGAUGAGGCCGUUAGGCCCGAA AAUGUUUU | 11507 | |
| 3718 | UUGAAGAUA UCCCGUUA | 2089 | UAACGGGA CUGAUGAGGCCGUUAGGCCCGAA AUCUUCAA | 11508 | |
| 3720 | GAAGAUAUC CCGUUAGA | 2090 | UCUAACGG CUGAUGAGGCCGUUAGGCCCGAA AUAUCUUC | 11509 | |
| 3725 | UAUCCCGUU AGAAGAAC | 2091 | GUUCUUCU CUGAUGAGGCCGUUAGGCCCGAA ACGGGAUA | 11510 | |
| 3726 | AUCCCGUUA GAAGAACC | 2092 | GGUUCUUC CUGAUGAGGCCGUUAGGCCCGAA AACGGGAU | 11511 | |
| 3741 | CCAGAAGUA AAAGUAAU | 2093 | AUUACUUU CUGAUGAGGCCGUUAGGCCCGAA ACUUCUGG | 11512 | |
| 3747 | GUAAAAGUA AUCCCAGA | 2094 | UCUGGGAU CUGAUGAGGCCGUUAGGCCCGAA ACUUUUAC | 11513 | |
| 3750 | AAAGUAAUC CCAGAUGA | 2095 | UCAUCUGG CUGAUGAGGCCGUUAGGCCCGAA AUUACUUU | 11514 | |
| 3778 | ACAGUGGUA UGGUUCUU | 2096 | AAGAACCA CUGAUGAGGCCGUUAGGCCCGAA ACCACUGU | 11515 | |
| 3783 | GGUAUGGUU CUUGCCUC | 2097 | GAGGCAAG CUGAUGAGGCCGUUAGGCCCGAA ACCAUACC | 11516 | |
| 3784 | GUAUGGUUC UUGCCUCA | 2098 | UGAGGCAA CUGAUGAGGCCGUUAGGCCCGAA AACCAUAC | 11517 | |
| 3786 | AUGGUUCUU GCCUCAGA | 2099 | UCUGAGGC CUGAUGAGGCCGUUAGGCCCGAA AGAACCAU | 11518 | |
| 3791 | UCUUGCCUC AGAAGAGC | 2100 | GCUCUUCU CUGAUGAGGCCGUUAGGCCCGAA AGGCAAGA | 11519 | |
| 3808 | UGAAAACUU UGGAAGAC | 2101 | GUCUUCCA CUGAUGAGGCCGUUAGGCCCGAA AGUUUUCA | 11520 | |
| 3809 | CAAAACUUU GGAAGACA | 2102 | UGUCUUCC CUGAUGAGGCCGUUAGGCCCGAA AAGUUUUC | 11521 | |
| 3827 | AACCAAAUU AUCUCCAU | 2103 | AUGGAGAU CUGAUGAGGCCGUUAGGCCCGAA AUUUGGUU | 11522 | |
| 3828 | ACCAAAUUA UCUCCAUC | 2104 | GAUGGAGA CUGAUGAGGCCGUUAGGCCCGAA AAUUUGGU | 11523 | |
| 3830 | CAAAUUAUC UCCAUCUU | 2105 | AAGAUGGA CUGAUGAGGCCGUUAGGCCCGAA AUAAUUUG | 11524 | |
| 3832 | AAUUAUCUC CAUCUUUU | 2106 | AAAAGAUG CUGAUGAGGCCGUUAGGCCCGAA AGAUAAUU | 11525 | |
| 3836 | AUCUCCAUC UUUUGGUG | 2107 | CACCAAAA CUGAUGAGGCCGUUAGGCCCGAA AUGGAGAG | 11526 | |
| 3838 | CUCCAUCUU UUGGUGGA | 2108 | UCCACCAA CUGAUGAGGCCGUUAGGCCCGAA AGAUGGAG | 11527 | |
| 3839 | UCCAUCUUU UGGUGGAA | 2109 | UUCCACCA CUGAUGAGGCCGUUAGGCCCGAA AAGAUGGA | 11528 | |
| 3840 | CCAUCUUUU GGUGGAAU | 2110 | AUUCCACC CUGAUGAGGCCGUUAGGCCCGAA AAAGAUGG | 11529 | |
| 3872 | CAGGGAGUC UGUGGCAU | 2111 | AUGCCACA CUGAUGAGGCCGUUAGGCCCGAA ACUCCCUG | 11530 | |
| 3881 | UGUGGCAUC UGAAGGCU | 2112 | AGCCUUCA CUGAUGAGGCCGUUAGGCCCGAA AUGCCACA | 11531 | |
| 3890 | UGAAGGCUC AAACCAGA | 2113 | UCUGGUUU CUGAUGAGGCCGUUAGGCCCGAA AGCCUUCA | 11532 | |
| 3908 | AAGCGGCUA CCAGUCCG | 2114 | CGGACUGG CUGAUGAGGCCGUUAGGCCCGAA AGCCGCUU | 11533 | |
| 3914 | CUACCAGUC CGGAUAUC | 2115 | GAUAUCCG CUGAUGAGGCCGUUAGGCCCGAA ACUGGUAG | 11534 | |
| 3920 | GUCCGGAUA UCACUCCG | 2116 | CGGAGUGA CUGAUGAGGCCGUUAGGCCCGAA AUCCCGAC | 11535 | |
| 3922 | CCGGAUAUC ACUCCGAU | 2117 | AUCGGAGU CUGAUGAGGCCGUUAGGCCCGAA AUAUCCGG | 11536 | |
| 3926 | AUAUCACUC CGAUGACA | 2118 | UGUCAUCG CUGAUGAGGCCGUUAGGCCCGAA AGUGAUAU | 11537 | |
| 3950 | CACCGUGUA CUCCAGUG | 2119 | CACUGGAG CUGAUGAGGCCGUUAGGCCCGAA ACACGGUG | 11538 | |
| 3953 | CGUGUACUC CAGUGAGG | 2120 | CCUCACUG CUGAUGAGGCCGUUAGGCCCGAA AGUACACG | 11539 | |
| 3972 | GCAGAACUU UUAAAGCU | 2121 | AGCUUUAA CUGAUGAGGCCGUUAGGCCCGAA AGUUCUGC | 11540 | |
| 3973 | CAGAACUUU UAAAGCUG | 2122 | CAGCUUUA CUGAUGAGGCCGUUAGGCCCGAA AAGUUCUG | 11541 | |
| 3974 | AGAACUUUU AAAGCUGA | 2123 | UCAGCUUU CUGAUGAGGCCGUUAGGCCCGAA AAAGUUCU | 11542 | |
| 3975 | GAACUUUUA AAGCUGAU | 2124 | AUCAGCUU CUGAUGAGGCCGUUAGGCCCGAA AAAAGUUC | 11543 | |
| 3984 | AAGCUGAUA GAGAUUGG | 2125 | CCAAUCUC CUGAUGAGGCCGUUAGGCCCGAA AUCAGCUU | 11544 | |
| 3990 | AUAGAGAUU GGAGUGCA | 2126 | UGCACUCC CUGAUGAGGCCGUUAGGCCCGAA AUCUCUAU | 11545 | |
| 4006 | AAACCGGUA GCACAGCC | 2127 | GGCUGUGC CUGAUGAGGCCGUUAGGCCCGAA ACCGGUUU | 11546 | |
| 4020 | GCCCAGAUU CUCCAGCC | 2128 | GGCUGGAG CUGAUGAGGCCGUUAGGCCCGAA AUCUGGGC | 11547 | |
| 4021 | CCCAGAUUC UCCAGCCU | 2129 | AGGCUGGA CUGAUGAGGCCGUUAGGCCCGAA AAUCUGGG | 11548 | |
| 4023 | CAGAUUCUC CAGCCUGA | 2130 | UCAGCCUG CUGAUGAGGCCGUUAGGCCCGAA AGAAUCUG | 11549 | |
| 4052 | ACUGAGCUC UCCUCCUG | 2131 | CAGGAGGA CUGAUGAGGCCGUUAGGCCCGAA AGCUCAGU | 11550 | |
| 4054 | UGAGCUCUC CUCCUGUU | 2132 | AACAGGAG CUGAUGAGGCCGUUAGGCCCGAA AGAGCUCA | 11551 | |
| 4057 | GCUCUCCUC CUGUUUAA | 2133 | UUAAACAG CUGAUGAGGCCGUUAGGCCCGAA AGGAGAGC | 11552 | |
| 4062 | CCUCCUGUU UAAAAGGA | 2134 | UCCUUUUA CUGAUGAGGCCGUUAGGCCCGAA ACAGGAGG | 11553 | |
| 4063 | CUCCUGUUU AAAAGGAA | 2135 | UUCCUUUU CUGAUGAGGCCGUUAGGCCCGAA AACAGGAG | 11554 | |
| 4064 | UCCUGUUUA AAAGGAAG | 2136 | CUUCCUUU CUGAUGAGGCCGUUAGGCCCGAA AAACAGGA | 11555 | |
| 4076 | GGAAGCAUC CACACCCC | 2137 | GGGGUGUG CUGAUGAGGCCGUUAGGCCCGAA AUGCUUCC | 11556 | |
| 4089 | CCCCAACUC CCGGACAU | 2138 | AUGUCCGG CUGAUGAGGCCGUUAGGCCCGAA AGUUGGGG | 11557 | |
| 4098 | CCGGACAUC ACAUGAGA | 2139 | UCUCAUGU CUGAUGAGGCCGUUAGGCCCGAA AUGUCCGG | 11558 | |
| 4110 | UGAGAGGUC UGCUCAGA | 2140 | UCUGAGCA CUGAUGAGGCCGUUAGGCCCGAA ACCUCUCA | 11559 | |
| 4115 | GGUCUGCUC AGAUUUUG | 2141 | CAAAAUCU CUGAUGAGGCCGUUAGGCCCGAA AGCAGACC | 11560 | |
| 4120 | GCUCAGAUU UUGAUGUG | 2142 | CACUUCAA CUGAUGAGGCCGUUAGGCCCGAA AUCUGAGC | 11561 | |
| 4121 | CUCAGAUUU UGAAGUGU | 2143 | ACACUUCA CUGAUGAGGCCGUUAGGCCCGAA AAUCUGAG | 11562 | |
| 4122 | UCAGAUUUU GAAGUGUU | 2144 | AACACUUC CUGAUGAGGCCGUUAGGCCCGAA AAAUCUGA | 11563 | |
| 4130 | UGAAGUGUU GUUCUUUC | 2145 | GAAAGAAC CUGAUGAGGCCGUUAGGCCCGAA ACACUUCA | 11564 | |
| 4133 | AGUGUUGUU CUUUCCAC | 2146 | GUGGAAAG CUGAUGAGGCCGUUAGGCCCGAA ACAACACU | 11565 | |
| 4134 | GUGUUGUUC UUUCCACC | 2147 | GGUGGAAA CUGAUGAGGCCGUUAGGCCCGAA AACAACAC | 11566 | |
| 4136 | GUUGUUCUU UCCACCAG | 2148 | CUGGUGGA CUGAUGAGGCCGUUAGGCCCGAA AGAACAAC | 11567 | |
| 4137 | UUGUUCUUU CCACCAGC | 2149 | GCUGGUGG CUGAUGAGGCCGUUAGGCCCGAA AAGAACAA | 11568 | |
| 4138 | UGUUCUUUC CACCAGCA | 2150 | UGCUGGUG CUGAUGAGGCCGUUAGGCCCGAA AAAGAACA | 11569 | |
| 4153 | CAGGAAGUA GCCGCAUU | 2151 | AAUGCGGC CUGAUGAGGCCGUUAGGCCCGAA ACUUCCUG | 11570 | |
| 4161 | AGCCGCAUU UGAUUUUC | 2152 | GAAAAUCA CUGAUGAGGCCGUUAGGCCCGAA AUGCGGCU | 11571 | |
| 4162 | GCCGCAUUU GAUUUUCA | 2153 | UGAAAAUC CUGAUGAGGCCGUUAGGCCCGAA AAUGCGGC | 11572 | |
| 4166 | CAUUUGAUU UUCAUUUC | 2154 | GAAAUGAA CUGAUGAGGCCGUUAGGCCCGAA AUCAAAUG | 11573 | |
| 4167 | AUUUGAUUU UCAUUUCG | 2155 | CGAAAUGA CUGAUGAGGCCGUUAGGCCCGAA AAUCAAAU | 11574 | |
| 4168 | UUUGAUUUU CAUUUCGA | 2156 | UCGAAAUG CUGAUGAGGCCGUUAGGCCCGAA AAAUCAAA | 11575 | |
| 4169 | UUGAUUUUC AUUUCCAC | 2157 | GUCGAAAU CUGAUGAGGCCGUUAGGCCCGAA AAAAUCAA | 11576 | |
| 4172 | AUUUUCAUU UCGACAAC | 2158 | GUUGUCGA CUGAUGAGGCCGUUAGGCCCGAA AUGAAAAU | 11577 | |
| 4173 | UUUUCAUUU CGACAACA | 2159 | UGUUGUCG CUGAUGAGGCCGUUAGGCCCGAA AAUGAAAA | 11578 | |
| 4174 | UUUCAUUUC GACAACAG | 2160 | CUGUUGUC CUGAUGAGGCCGUUAGGCCCGAA AAAUGAAA | 11579 | |
| 4194 | AAGGACCUC GGACUGCA | 2161 | UGCAGUCC CUGAUGAGGCCGUUAGGCCCGAA AGCUCCUU | 11580 | |
| 4214 | AGCCAGCUC UUCUAGGC | 2162 | GCCUAGAA CUGAUGAGGCCGUUAGGCCCGAA AGCUGGCU | 11581 | |
| 4216 | CCAGCUCUU CUAGGCUU | 2163 | AAGCCUAG CUGAUGAGGCCGUUAGGCCCGAA AGAGCUGG | 11582 | |
| 4217 | CAGCUCUUC UAGGCUUG | 2164 | CAAGCCUA CUGAUGAGGCCGUUAGGCCCGAA AAGAGCUG | 11583 | |
| 4219 | GCUCUUCUA GGCUUGUG | 2165 | CACAAGCC CUGAUGAGGCCGUUAGGCCCGAA AGAAGAGC | 11584 | |

Where “X” represents stem II region of a HH ribozyme (Hertel et al., 1992 Nucleic Acids Res. 20 3252).
The length of stem II may be ≧ 2 base-pairs.
Underlined region can be any X sequence or linker, as described herein.

TABLE V
Human KDR VEGF Receptor—Hairpin Ribozyme and Substrate Sequences

| | | Seq ID | | |
|---|---|---|---|---|
| Pos | Substrate | No | Hairpin Ribozyme Sequence | Seq ID No |
| 11 | AGGUGCU GCU GGCCGUCG | 2166 | CGACGGCC AGAA GCACCU ACCAGAGAAACACACGUUGUGGUACAUUACCUGGUA | 11585 |
| 18 | GCUGGCC GUC GCCCUGUG | 2167 | CACAGGGC AGAA GCCAGC ACCAGAGAAACACACGUUGUGGUACAUUACCUGGUA | 11586 |
| 51 | CCGGGCC GCC UCUGUGGG | 2168 | CCCACAGA AGAA GCCCGG ACCAGAGAAACACACGUUGUGGUACAUUACCUGGUA | 11587 |
| 86 | UUGAUCU GCC CAGUCUCA | 2169 | UGAGCCUG AGAA GAUCAA ACCAGAGAAACACACGUUGUGGUACAUUACCUGGUA | 11588 |
| 318 | GGAAACU GAC UUGGCCUC | 2170 | GAGGCCAA AGAA GUUUCC ACCAGAGAAACACACGUUGUGGUACAUUACCUGGUA | 11589 |
| 358 | GAUUACA GAU CUCCAUUU | 2171 | AAAUGGAG AGAA GUAAUC ACCAGAGAAACACACGUUGUGGUACAUUACCUGGUA | 11590 |
| 510 | UGUUCCU GAU GGUAACAG | 2172 | CUGUUACC AGAA GGAACA ACCAGAGAAACACACGUUGUGGUACAUUACCUGGUA | 11591 |
| 623 | GUUACCA GUC UAUUAUGU | 2173 | ACAUAAUA AGAA GGUAAC ACCAGAGAAACACACGUUGUGGUACAUUACCUGGUA | 11592 |
| 683 | UGAGUCC GUC UCAUGGAA | 2174 | UUCCAUGA AGAA GACUCA ACCAGAGAAACACACGUUGUGGUACAUUACCUGGUA | 11593 |
| 705 | ACUAUCU GUU GGAGAAAA | 2175 | UUUUCUCC AGAA GAUAGU ACCAGAGAAACACACGUUGUGGUACAUUACCUGGUA | 11594 |
| 833 | AAACCCA GUC UGGGAGUG | 2176 | CACUCCCA AGAA GGGUUU ACCAGAGAAACACACGUUGUGGUACAUUACCUGGUA | 11595 |
| 932 | GUGGGCU GAU GACCAAGA | 2177 | UCUUGGUC AGAA GCCCAC ACCAGAGAAACACACGUUGUGGUACAUUACCUGGUA | 11596 |
| 1142 | AUGUACU GAC GAUUAUGG | 2178 | CCAUAAUC AGAA GUACAU ACCAGAGAAACACACGUUGUGGUACAUUACCUGGUA | 11597 |
| 1259 | CACCCCA GAU UGGUGAGA | 2179 | UCUCACCA AGAA GGGGUG ACCAGAGAAACACACGUUGUGGUACAUUACCUGGUA | 11598 |
| 1332 | AUGUACG GUC UAUGCCAU | 2180 | AUGGCAUA AGAA GUACAU ACCAGAGAAACACACGUUGUGGUACAUUACCUGGUA | 11599 |
| 1376 | AUUGGCA GUU GGAGGAAG | 2181 | CUUCCUCC AGAA GCCAAU ACCAGAGAAACACACGUUGUGGUACAUUACCUGGUA | 11600 |
| 1413 | CCAAGCU GUC UCAGUGAC | 2182 | GUCACUGA AGAA GCUUGG ACCAGAGAAACACACGUUGUGGUACAUUACCUGGUA | 11601 |
| 1569 | UGUGUCA GCU UUGUACAA | 2183 | UUGUACAA AGAA GACACA ACCAGAGAAACACACGUUGUGGUACAUUACCUGGUA | 11602 |
| 1673 | ACAUGCA GCC CACUGAGC | 2184 | GCUCAGUG AGAA GCAUGU ACCAGAGAAACACACGUUGUGGUACAUUACCUGGUA | 11603 |
| 1717 | GCAGACA GAU CUACGUUU | 2185 | AAACGUAG AGAA GUCUGC ACCAGAGAAACACACGUUGUGGUACAUUACCUGGUA | 11604 |
| 1760 | GCCCACA GCC UCUGCCAA | 2186 | UUGGCAGA AGAA GUGGGC ACCAGAGAAACACACGUUGUGGUACAUUACCUGGUA | 11605 |
| 1797 | CACACCU GUU UGCAAGAA | 2187 | UUCUUGCA AGAA GGUGUG ACCAGAGAAACACACGUUGUGGUACAUUACCUGGUA | 11606 |
| 1918 | UAUGUCU GCC UUGCUCAA | 2188 | UUGAGCAA AGAA GACAUA ACCAGAGAAACACACGUUGUGGUACAUUACCUGGUA | 11607 |
| 1967 | UCAGGCA GCU CACAGUCC | 2189 | GGACUGUG AGAA GCCUGA ACCAGAGAAACACACGUUGUGGUACAUUACCUGGUA | 11608 |
| 1974 | GCUCACA GUC CUAGAGCG | 2190 | CGCUCUAG AGAA GUGAGC ACCAGAGAAACACACGUUGUGGUACAUUACCUGGUA | 11609 |
| 2021 | AGAAUCA GAC GACAAGUA | 2191 | UACUUGUC AGAA GAUUCU ACCAGAGAAACACACGUUGUGGUACAUUACCUGGUA | 11610 |
| 2084 | CUCCACA GAU CAUGUGGU | 2192 | ACCACAUG AGAA GUGGAG ACCAGAGAAACACACGUUGUGGUACAUUACCUGGUA | 11611 |
| 2418 | GGAUCCA GAU GAACUCCC | 2193 | GGGAGUUC AGAA GGAUCC ACCAGAGAAACACACGUUGUGGUACAUUACCUGGUA | 11612 |
| 2453 | AACGACU GCC UUAUGAUG | 2194 | CAUCAUAA AGAA GUCGUU ACCAGAGAAACACACGUUGUGGUACAUUACCUGGUA | 11613 |
| 2492 | GAGACCG GCU GAACCUAG | 2195 | CUAGGUUC AGAA GGUCUC ACCAGAGAAACACACGUUGUGGUACAUUACCUGGUA | 11614 |
| 2547 | UGAAGCA GAU GCCUUUGG | 2196 | CCAAAGGC AGAA GCUUCA ACCAGAGAAACACACGUUGUGGUACAUUACCUGGUA | 11615 |
| 2765 | GAAACCU GUC CACUUACC | 2197 | GGUAAGUG AGAA GGUUCA ACCAGAGAAACACACGUUGUGGUACAUUACCUGGUA | 11616 |
| 2914 | UCAGCCA GCU CUGGAUUU | 2198 | AAAUCCAG AGAA GGCUGA ACCAGAGAAACACACGUUGUGGUACAUUACCUGGUA | 11617 |
| 2993 | ACUUCCU GAC CUUGGACC | 2199 | GCUCCAAG AGAA CGAAGU ACCAGAGAAACACACGUUGUGGUACAUUACCUGGUA | 11618 |
| 3019 | UGUUACA GCU UCCAAGUG | 2200 | CACUUGGA AGAA GUAACA ACCAGAGAAACACACGUUGUGGUACAUUACCUGGUA | 11619 |
| 3165 | AGAUCCA GAG GAUGUCAG | 2201 | CUGACAGA AGAA GGAUCU ACCAGAGAAACACACGUUGUGGUACAUUACCUGGUA | 11620 |
| 3378 | GGCCCCU GAU UAUACUAC | 2202 | GUAGUAUA AGAA GGGGCC ACCAGAGAAACACACGUUGUGGUACAUUACCUGGUA | 11621 |
| 3404 | UGUACCA GAC CAUGCUGG | 2203 | CCAGCAUG AGAA UGUACA ACCAGAGAAACACACGUUGUGGUACAUUACCUGGUA | 11622 |
| 3418 | CUGGACU GCU GGCACGGG | 2204 | CCCGUGCC AGAA GUCCAG ACCAGAGAAACACACGUUGUGGUACAUUACCUGGUA | 11623 |
| 3575 | UCUCUCU GCC UACCUCAC | 2205 | GUGAGGUA AGAA GAGAGA ACCAGAGAAACACACGUUGUGGUACAUUACCUGGUA | 11624 |
| 3588 | CUCACCU GUU UCCUGUAU | 2206 | AUACAGGA AGAA GGUGAG ACCAGAGAAACACACGUUGUGGUACAUUACCUGGUA | 11625 |
| 3689 | AGAGCCG GCC UGUGAGUG | 2207 | CACUCACA AGAA GGCUCU ACCAGAGAAACACACGUUGUGGUACAUUACCUGGUA | 11626 |
| 3753 | AAUCCCA GAU GACAACCA | 2208 | UGGUUGUC AGAA GCGAUU ACCAGAGAAACACACGUUGUGGUACAUUACCUGGUA | 11627 |
| 3764 | ACAACCA GAC GGACAGUG | 2209 | CACUGUCC AGAA GGUUGU ACCAGAGAAACACACGUUGUGGUACAUUACCUGGUA | 11628 |
| 3911 | GCUACCA GUC CGGAUAUC | 2210 | GAUAUCCG AGAA GGUAGC ACCAGAGAAACACACGUUGUGGUACAUUACCUGGUA | 11629 |
| 3927 | UCACUCC GAG GACACACA | 2211 | UCUGUGUC AGAA GAGUGA ACCAGAGAAACACACGUUGUGGUACAUUACCUGGUA | 11630 |
| 4011 | UAGCACA GCC CAGAUUCU | 2212 | AGAAUCUG AGAA GUGCUA ACCAGAGAAACACACGUUGUGGUACAUUACCUGGUA | 11631 |
| 4016 | CAGCCCA GAU UCUCCAGC | 2213 | GCUGGAGA AGAA GGGCUG ACCAGAGAAACACACGUUGUGGUACAUUACCUGGUA | 11632 |
| 4025 | GUCUCCA GCC UGACACGG | 2214 | CCGUGUCA AGAA GGAGAA ACCAGAGAAACACACGUUGUGGUACAUUACCUGGUA | 11633 |
| 4059 | UCCUCCU GUU UAAAAGGA | 2215 | UCCUUUUA AGAA GGAGGA ACCAGAGAAACACACGUUGUGGUACAUUACCUGGUA | 11634 |
| 4111 | GAGGUCU GCU CAGAUUUU | 2216 | AAAAUCUG AGAA GACCUC ACCAGAGAAACACACGUUGUGGUACAUUACCUGGUA | 11635 |
| 4116 | CUGCUCA GAU UUUGAAGU | 2217 | ACUUCAAA AGAA GAGCAG ACCAGAGAAACACACGUUGUGGUACAUUACCUGGUA | 11636 |
| 4195 | GACCUCG GAC UGCAGGGA | 2218 | UCCCUGCA AGAA GAGGUC ACCAGAGAAACACACGUUGUGGUACAUUACCUGGUA | 11637 |
| 4210 | GGAGCCA GCU CUUCUAGG | 2219 | CCUAGAAG AGAA GGCUCC ACCAGAGAAACACACGUUGUGGUACAUUACCUGGUA | 11638 |

TABLE VI
Mouse flk-1 VEGF Receptor—Hammerhead Ribozyme and Substrate Sequence

| Pos | Substrate | Seq ID No | HH Ribozyme Sequence | Seq ID No |
|---|---|---|---|---|
| 13 | GGGCGAAUU GGGUACGG | 2220 | CCGUACCC CUGAUGAGGCCGUUAGGCCGAA AUUCGCCC | 11639 |
| 18 | AAUUGGGUA CGGGACCC | 2221 | GGGUCCCG CUGAUGAGGCCGUUAGGCCGAA ACCCAAUU | 11640 |
| 31 | ACCCCCCUC GAGGUCGA | 2222 | UCGACCUC CUGAUGAGGCCGUUAGGCCGAA AGGGGGGU | 11641 |
| 37 | CUCGAGGUC GACGGUAU | 2223 | AUACCGUC CUGAUGAGGCCGUUAGGCCGAA ACCUCGAG | 11642 |
| 44 | UCGACGGUA UCGAUAAG | 2224 | CUUAUCGA CUGAUGAGGCCGUUAGGCCGAA ACCGUCGA | 11643 |
| 46 | GACGGUAUC GAUAAGCU | 2225 | AGCUUAUC CUGAUGAGGCCGUUAGGCCGAA AUACCGUC | 11644 |
| 50 | GUAUCGAUA AGCUUGAU | 2226 | AUCAAGCU CUGAUGAGGCCGUUAGGCCGAA AUCGAUAC | 11645 |
| 55 | GAUAAGCUU GAUAUCGA | 2227 | UCGAUAUC CUGAUGAGGCCGUUAGGCCGAA AGCUUAUC | 11646 |
| 59 | AGCUUGAUA UCGAAUUC | 2228 | GAAUUCGA CUGAUGAGGCCGUUAGGCCGAA AUCAAGCU | 11647 |
| 61 | CUUGAUAUC GAAUUCGG | 2229 | CCGAAUUC CUGAUGAGGCCGUUAGGCCGAA AUAUCAAG | 11648 |
| 66 | UAUCGAAUU CGGGCCCA | 2230 | UGGGCCCG CUGAUGAGGCCGUUAGGCCGAA AUUCGAUA | 11649 |
| 67 | AUCGAAUUC GGGCCCAG | 2231 | CUGGGCCC CUGAUGAGGCCGUUAGGCCGAA AAUUCGAU | 11650 |
| 83 | GACUGUGUC CCGCAGCC | 2232 | GGCUGCGG CUGAUGAGGCCGUUAGGCCGAA ACACAGUC | 11651 |
| 97 | GCCGGGAUA ACCUGGCU | 2233 | AGCCAGGU CUGAUGAGGCCGUUAGGCCGAA AUCCCGGC | 11652 |
| 114 | GACCCGAUU CCGCGGAC | 2234 | GUCCGCGG CUGAUGAGGCCGUUAGGCCGAA AUCGGGUC | 11653 |
| 115 | ACCCGAUUC CGCGGACA | 2235 | UGUCCGCG CUGAUGAGGCCGUUAGGCCGAA AAUCGGGU | 11654 |
| 169 | CCCGCGCUC UCCCCGGU | 2236 | ACCGGGGA CUGAUGAGGCCGUUAGGCCGAA AGCGCGGG | 11655 |
| 171 | CGCGCUCUC CCCGGUCU | 2237 | AGACCGGG CUGAUGAGGCCGUUAGGCCGAA AGACCGCG | 11656 |
| 178 | UCCCCGGUC UUGCGCUG | 2238 | CAGCGCAA CUGAUGAGGCCGUUAGGCCGAA ACCGGGGA | 11657 |
| 180 | CCCGGUCUU GCGCUGCG | 2239 | CGCAGCGC CUGAUGAGGCCGUUAGGCCGAA AGACCGGG | 11658 |
| 197 | GGGGCCAUA CCGCCUCU | 2240 | AGAGGCGG CUGAUGAGGCCGUUAGGCCGAA AUGGCCCC | 11659 |
| 204 | UACCGCCUC UGUGACUU | 2241 | AAGUCACA CUGAUGAGGCCGUUAGGCCGAA AGGCGGUA | 11660 |
| 212 | CUGUGACUU CUUUGCGG | 2242 | CCGCAAAG CUGAUGAGGCCGUUAGGCCGAA AGUCACAG | 11661 |
| 213 | UGUGACUUC UUUGCGGG | 2243 | CCCGCAAA CUGAUGAGGCCGUUAGGCCGAA AAGUCACA | 11662 |
| 215 | UGACUUCUU UGCGGGCC | 2244 | GGCCCGCA CUGAUGAGGCCGUUAGGCCGAA AGAAGUCA | 11663 |
| 216 | GACUUCUUU GCGGGCCA | 2245 | UGGCCCGC CUGAUGAGGCCGUUAGGCCGAA AAGAAGUC | 11664 |
| 241 | GAAGGAGUC UGUGCCUG | 2246 | CAGGCACA CUGAUGAGGCCGUUAGGCCGAA ACUCCUUC | 11665 |
| 262 | ACUGGGCUC UGUGCCCA | 2247 | UGGGCACA CUGAUGAGGCCGUUAGGCCGAA AGCCCAGU | 11666 |
| 306 | GCGCUGCUA GCUGUCGC | 2248 | GCGACAGC CUGAUGAGGCCGUUAGGCCGAA AGCAGCGC | 11667 |
| 312 | CUAGCUGUC GCUCUGUG | 2249 | CACAGAGC CUGAUGAGGCCGUUAGGCCGAA ACAGCUAG | 11668 |
| 316 | CUGUCGCUC UGUGGUUC | 2250 | GAACCACA CUGAUGAGGCCGUUAGGCCGAA AGCGACAG | 11669 |
| 323 | UCUGUGGUU CUGCGUGG | 2251 | CCACGCAG CUGAUGAGGCCGUUAGGCCGAA ACCACAGA | 11670 |
| 324 | CUGUGGUUC UGCGUGGA | 2252 | UCCACGCA CUGAUGAGGCCGUUAGGCCGAA AACCACAG | 11671 |
| 347 | AUCCUCCUC UGUGGGUU | 2253 | AACCCACA CUGAUGAGUCCGUUAGGCCGAA AGGCGGCU | 11672 |
| 355 | CUGUGGGUU UGACUGGC | 2254 | GOCAGUCA CUGAUGAGGCCGUUAGGCCGAA ACCCACAG | 11673 |
| 356 | UGUGGGUUU GACUGGCG | 2255 | CGCCAGUC CUGAUGAGGCCGUUAGGCCGAA AACCCACA | 11674 |
| 367 | CUGGCGAUU UUCUCCAU | 2256 | AUGGAGAA CUGAUGAGGCCGUUAGGCCGAA AUCGCCAG | 11675 |
| 368 | UGGCGAUUU UCUCCAUC | 2257 | GAUGGAGA CUGAUGAGGCCGUUAGGCCGAA AAUCGCCA | 11676 |
| 369 | GGCGAUUUU CUCCAUCC | 2258 | GGAUGGAG CUGAUGAGGCCGUUAGGCCGAA AAAUCGCC | 11677 |
| 370 | GCGAUUUUC UCCAUCCC | 2259 | GGGAUGGA CUGAUGAGGCCGUUAGGCCGAA AAAAUCGC | 11678 |
| 372 | GAUUUUCUC CAUCCCCC | 2260 | GGGGGAUG CUGAUGAGGCCGUUAGGCCGAA AGAAAAUC | 11679 |
| 376 | UUCUCCAUC CCCCCAAG | 2261 | CUUGGGGG CUGAUGAGGCCGUUAGGCCGAA AUGGAGAA | 11680 |
| 387 | CCCAAGCUC AGCACACA | 2262 | UGUGUGCU CUGAUGAGGCCGUUAGGCCGAA AGCUUGGG | 11681 |
| 405 | AAAGACAUA CUGACAAU | 2263 | AUUGUCAG CUGAUGAGGCCGUUAGGCCGAA AUGUCUUU | 11682 |
| 414 | CUGACAATU UUGGCAAA | 2264 | UUUGCCAA CUGAUGAGGCCGUUAGGCCGAA AUUGUCAG | 11683 |
| 415 | UGACAAUUU UGGCAAAU | 2265 | AUUUGCCA CUGAUGAGGCCGUUAGGCCGAA AAUUGUCA | 11684 |
| 416 | GACAAUUUU GGCAAAUA | 2266 | UAUUUGCC CUGAUGAGGCCGUUAGGCCGAA AAAUUGUC | 11685 |
| 424 | UGGCAAAUA CAACCCUU | 2267 | AAGGGUUG CUGAUGAGGCCGUUAGGCCGAA AUUUGCCA | 11686 |
| 432 | ACAACCCUU CAGAUUAC | 2268 | GUAAUCUG CUGAUGAGGCCGUUAGGCCGAA AGGGUUGU | 11687 |
| 433 | CAACCCUUC AGAUUACU | 2269 | AGUAAUCU CUGAUGAGGCCGUUAGGCCGAA AAGGGUUG | 11688 |
| 438 | CUUCAGAUU ACUUGCAG | 2270 | CUGCAAGU CUGAUGAGGCCGUUAGGCCCAA AUCUGAAG | 11689 |
| 439 | UUCAGAUUA CUUGCAGG | 2271 | CCUGCAAG CUGAUGAGGCCGUUAGGCCGAA AAUCUGAA | 11690 |
| 442 | AGAUUACUU GCAGGCGA | 2272 | UCCCCUGC CUGAUGAGGCCGUUAGGCCGAA ACUAAUCU | 11691 |
| 471 | GACUGGCUU UGGCCCAA | 1534 | UUGGGCCA CUGAUGAGGCCGUUAGGCCGAA AGCCAGUC | 10953 |
| 472 | ACUGGCUUU GGCCCAAU | 1535 | AUUGGGCC CUGAUGAGGCCGUUAGGCCGAA AAGCCAGU | 10954 |
| 484 | CCAAUGCUC AGCGUGAU | 2273 | AUCACGCU CUGAUGAGGCCGUUAGGCCGAA AGCAUUGG | 11692 |
| 493 | AGCGUGAUU CUGAGGAA | 2274 | UUCCUCAG CUGAUGAGGCCGUUAGGCCGAA AUCACGCU | 11693 |
| 494 | GCGUGAUUC UGAGGAAA | 2275 | UUUCCUCA CUGAUGAGGCCGUUAGGCCGAA AAUCACGC | 11694 |
| 507 | GAAAGGGUA UUGGUGAC | 2276 | GUCACCAA CUGAUGAGGCCGUUAGGCCGAA ACCCUUUC | 11695 |
| 509 | AAGGGUAUU GGUGACUG | 2277 | CAGUCACC CUGAUGAGGCCGUUAGGCCGAA AUACCCUU | 11696 |
| 538 | GUGACAGUA UCUUCUGC | 2278 | GCAGAAGA CUGAUGAGGCCGUUAGGCCGAA ACUGUCAC | 11697 |
| 540 | GACAGUAUC UUCUGCAA | 2279 | UUGCAGAA CUGAUGAGGCCGUUAGGCCGAA AUACUGUC | 11698 |
| 542 | CAGUAUCUU CUGCAAAA | 2280 | UUUUGCAG CUGAUGAGGCCGUUAGGCCGAA AGAUACUG | 11699 |
| 543 | AGUAUCUUC UGCAAAAC | 2281 | GUUUUGCA CUGAUGAGGCCGUUAGGCCGAA AAGAUACU | 11700 |
| 555 | AAAACACUC ACCAUUCC | 2282 | GGAAUGGU CUGAUGAGGCCGUUAGGCCGAA AGUGUUUU | 11701 |
| 561 | CUCACCAUU CCCAGGGU | 2283 | ACCCUGGG CUGAUGAGGCCGUUAGGCCGAA AUGGUGAG | 11702 |
| 562 | UCACCAUUC CCAGGGUG | 2284 | CACCCUGG CUGAUGAGGCCGUUAGGCCGAA AAUGGUGA | 11703 |
| 573 | AGGGUGGUU GGAAAUGA | 2285 | UCAUUUCC CUGAUGAGGCCGUUAGGCCGAA ACCACCCU | 11704 |
| 583 | GAAAUGAUA CUGGAGCC | 2286 | GGCUCCAG CUGAUGAGGCCGUUAGGCCGAA AUCAUUUC | 11705 |
| 593 | UGGAGCCUA CAAGUGCU | 1546 | AGCACUUG CUGAUGAGGCCGUUAGGCCGAA AGGCUCCA | 10965 |
| 602 | CAAGUGCUC GUACCGGG | 2287 | CCCGGUAC CUGAUGAGGCCGUUAGGCCGAA AGCACTUG | 11706 |
| 605 | GUGCUCGUA CCGGGACG | 2288 | CGUCCCGG CUGAUGAGGCCGUUAGGCCGAA ACCACCAC | 11707 |
| 615 | CGGGACGUC GACAUAGC | 2289 | GCUAUGUC CUGAUGAGGCCGUUAGGCCGAA ACGUCCCG | 11708 |
| 621 | GUCGACAUA GCCUCCAC | 2290 | GUGGAGGC CUGAUGAGGCCGUUAGGCCGAA AUGUCGAC | 11709 |
| 626 | CAUAGCCUC CACUGUUU | 2291 | AAACAGUG CUGAUGAGGCCGUUAGGCCGAA AGGCUAUG | 11710 |
| 633 | UCCACUGUU UAUGUCUA | 2292 | UAGACAUA CUGAUGAGGCCGUUAGGCCGAA ACAGUGGA | 11711 |
| 634 | CCACUGUUU AUGUCUAU | 2293 | AUAGACAU CUGAUGAGGCCGUUAGGCCGAA AACAGUGG | 11712 |
| 635 | CACUGUUUA UGUCUAUG | 2294 | CAUAGACA CUGAUGAGGCCGUUAGGCCGAA AAACAGUG | 11713 |
| 639 | GUUUAUGUC UAUGUUCG | 2295 | CGAACAUA CUGAUGAGGCCGUUAGGCCGAA ACAUAAAC | 11714 |
| 641 | UUAUGUCUA UGUUCGAG | 2296 | CUCGAACA CUGAUGAGGCCGUUAGGCCGAA AGACAUAA | 11715 |
| 645 | GUCUAUGUU CGAGAUUA | 2297 | UAAUCUCG CUGAUGAGGCCGUUAGGCCGAA ACAUAGAC | 11716 |
| 646 | UCUAUGUUC GAGAUUAC | 2298 | GUAAUCUC CUGAUGAGGCCGUUAGGCCGAA AACAUAGA | 11717 |
| 652 | UUCGAGAUU ACAGAUCA | 2299 | UGAUCUGU CUGAUGAGGCCGUUAGGCCGAA AUCUCGAA | 11718 |
| 653 | UCGAGAUUA CAGAUCAC | 2300 | GUGAUCUG CUGAUGAGGCCGUUAGGCCGAA AAUCUCGA | 11719 |
| 659 | HUACAGAUC ACCAUUCA | 2301 | UGAAUGGU CUGAUGAGGCCGUUAGGCCGAA AUCUGUAA | 11720 |
| 665 | AUCACCAUU CAUCGCCU | 2302 | AGGCGAUG CUGAUGAGGCCGUUAGGCCGAA AUGGUGAU | 11721 |
| 666 | UCACCAUUC AUCGCCUC | 2303 | GAGGCGAU CUGAUGAGGCCGUUAGGCCGAA AAUGGUGA | 11722 |
| 669 | CCAUUCAUC GCCUCUGU | 2304 | ACAGAGGC CUGAUGAGGCCGUUAGGCCGAA AUGAAUGG | 11723 |
| 674 | CAUCGCCUC UGUCAGUG | 2305 | CACUGACA CUGAUGAGGCCGUUAGGCCGAA AGGCGAUG | 11724 |
| 678 | GCCUCUGUC AGUGACCA | 2306 | UGGUCACU CUGAUGAGGCCGUUAGGCCGAA ACAGAGGC | 11725 |
| 696 | CAUGGCAUC GUGUACAU | 2307 | AUGUACAC CUGAUGAGGCCGUUAGGCCGAA AUGCCAUG | 11726 |
| 701 | CAUCGUGUA CAUCACCG | 2308 | CGGUGAUG CUGAUGAGGCCGUUAGGCCGAA ACACGAUG | 11727 |
| 705 | GUGUACAUC ACCGAGAA | 2309 | UUCUCGGU CUGAUGAGGCCGUUAGGCCGAA AUGUACAC | 11728 |
| 735 | GUGGUGAUC CCCUGCCG | 2310 | CGGCAGGG CUGAUGAGGCCGUUAGGCCGAA AUCACCAC | 11729 |
| 749 | CCGAGGGUC GAUUUCAA | 2311 | UUGAAAUC CUGAUGAGGCCGUUAGGCCGAA ACCCUCGG | 11730 |
| 753 | GGGUCGAUU UCAAACCU | 2312 | AGGUUUGA CUGAUGAGGCCGUUAGGCCGAA AUCGACCC | 11731 |
| 754 | GGUCGAUUU CAAACCUC | 2313 | GAGGUUUG CUGAUGAGGCCGUUAGGCCGAA AAUCGACC | 11732 |
| 755 | GUCGAUUUC AAACCUCA | 2314 | UGAGGUUU CUGAUGAGGCCGUUAGGCCGAA AAAUCGAC | 11733 |
| 762 | UCAAACCUC AAUGUGUC | 2315 | GACACAUU CUGAUGAGGCCGUUAGGCCGAA AGGUUUCA | 11734 |
| 770 | CAAUGUGUC UCUUUGCG | 2316 | CGCAAAGA CUGAUGAGGCCGUUAGGCCGAA ACACAUUG | 11735 |
| 772 | AUGUGUCUC UUUGCGCU | 2317 | AGCGCAAA CUGAUGAGGCCGUUAGGCCGAA AGACACAU | 11736 |
| 774 | GUGUCUCUU UGCGCUAG | 2318 | CUAGCGCA CUGAUGAGGCCGUUAGGCCGAA AGAGACAC | 11737 |
| 775 | UGUCUCUUU GCGCUAGG | 2319 | CCUAGCGC CUGAUGAGGCCGUUAGGCCGAA AAGAGACA | 11738 |
| 781 | UUUGCGCUA GGUAUCCA | 2320 | UGGAUACC CUGAUGAGGCCGUUAGGCCGAA AGCGCAAA | 11739 |
| 785 | CGCUAGGUA UCCAGAAA | 2321 | UCUCUGGA CUGAUGAGGCCGUUAGGCCGAA ACCUAGCG | 11740 |
| 787 | CUAGGUAUC CAGAAAAG | 2322 | CUUUUCUG CUGAUGAGGCCGUUAGGCCGAA AUACCUAG | 11741 |
| 800 | AAAGAGAUU UGUUCCGG | 2323 | CCGGAACA CUGAUGAGGCCGUUAGGCCGAA AUCUCUUU | 11742 |
| 801 | AAGAGAUUU GUUCCGGA | 2324 | UCCGGAAC CUGAUGAGGCCGUUAGGCCGAA AAUCUCUU | 11743 |
| 804 | AGAUUUGUU CCGGAUGG | 2325 | CCAUCCGG CUGAUGAGGCCGUUAGGCCGAA ACAAAUCU | 11744 |
| 805 | GAUUUGUUC CGGAUGGA | 2326 | UCCAUCCG CUGAUGAGGCCGUUAGGCCGAA AACAAAUC | 11745 |
| 822 | AACAGAAUU UCCUGGGA | 1595 | UCCCAGGA CUGAUGAGGCCGUUAGGCCGAA AUUCUGUU | 11014 |
| 823 | ACAGAAUUU CCUGGGAC | 1596 | GUCCCAGG CUGAUGAGGCCGUUAGGCCGAA AAUUCUGU | 11015 |
| 824 | CAGAAUUUC CUGGGACA | 1597 | UGUCCCAG CUGAUGAGGCCGUUAGGCCGAA AAAUUCUG | 11016 |
| 840 | AGCGAGAUA GGCUUUAC | 2327 | GUAAAGCC CUGAUGAGGCCGUUAGGCCGAA AUCUCGCU | 11746 |
| 845 | GAUAGGCUU UACUCUCC | 2328 | GGAGAGUA CUGAUGAGGCCGUUAGGCCGAA AGCCUAUC | 11747 |
| 846 | AUAGGCUUU ACUCUCCC | 2329 | GGGAGAGU CUGAUGAGGCCGUUAGGCCGAA AAGCCUAU | 11748 |
| 847 | UAGGCUUUA CUCUCCCC | 2330 | GGGGAGAG CUGAUGAGGCCGUUAGGCCGAA AAAGCCUA | 11749 |
| 850 | GCUUUACUC UCCCCAGU | 2331 | ACUGGGGA CUGAUGAGGCCGUUAGGCCGAA AGUAAAGC | 11750 |
| 852 | UUUACUCUC CCCAGUUA | 2332 | UAACUGGG CUGAUGAGGCCGUUAGGCCGAA AGAGUAAA | 11751 |
| 859 | UCCCCAGUU ACAUGAUC | 2333 | GAUCAUGU CUGAUGAGGCCGUUAGGCCGAA ACUGGGGA | 11752 |
| 860 | CCCCAGUUA CAUGAUCA | 2334 | UGAUCAUG CUGAUGAGGCCGUUAGGCCGAA AACUGGGG | 11753 |
| 867 | UACAUGAUC AGCUAUGC | 1605 | GCAUAGCU CUGAUGAGGCCGUUAGGCCGAA AUCAUGUA | 11024 |
| 872 | GAUCAGCUA UGCCGGCA | 2335 | UGCCGGCA CUGAUGAGGCCGUUAGGCCGAA AGCUGAUC | 11754 |
| 885 | GGCAUGGUC UUCUGUGA | 1607 | UCACAGAA CUGAUGAGGCCGUUAGGCCGAA ACCAUGCC | 11026 |
| 887 | CAUGGUCUU CUGUGAGG | 2336 | CCUCACAG CUGAUGAGGCCGUUAGGCCGAA AGACCAUG | 11755 |
| 888 | AUGGUCUUC UGUGAGGC | 2337 | GCCUCACA CUGAUGAGGCCGUUAGGCCGAA AAGACCAU | 11756 |
| 903 | GCAAAGAUC AAUGAUGA | 2338 | UCAUCAUU CUGAUGAGGCCGUUAGGCCGAA AUCUUUGC | 11757 |
| 917 | UGAAACCUA UCAGUCUA | 2339 | UAGACUGA CUGAUGAGGCCGUUAGGCCGAA AGGUUUCA | 11758 |
| 919 | AAACCUAUC AGUCUAUC | 2340 | GAUAGACU CUGAUGAGGCCGUUAGGCCGAA AUAGGUUU | 11759 |
| 923 | CUAUCAGUC UAUCAUGU | 2341 | ACAUGAUA CUGAUGAGGCCGUUAGGCCGAA ACUGAUAG | 11760 |
| 925 | AUCAGUCUA UCAUGUAC | 2342 | GUACAUGA CUGAUGAGGCCGUUAGGCCGAA AGACUGAU | 11761 |
| 927 | CAGUCUAUC AUGUACAU | 2343 | AUGUACAU CUGAUGAGGCCGUUAGGCCGAA AUAGACUG | 11035 |
| 932 | UAUCAUGUA CAUAGUUG | 2344 | CAACUAUG CUGAUGAGGCCGUUAGGCCGAA ACAUGAUA | 11762 |
| 936 | AUGUACAUA GUUGUGGU | 2345 | ACCACAAC CUGAUGAGGCCGUUAGGCCGAA AUGUACAU | 11763 |
| 939 | UACAUAGUU GUGGUUGU | 2346 | ACAACCAC CUGAUGAGGCCGUUAGGCCGAA ACUAUGUA | 11764 |
| 945 | GUUGUGGUU GUAGGAUA | 2347 | UAUCCUAC CUGAUGAGGCCGUUAGGCCGAA ACCACAAC | 11765 |
| 948 | GUGGUUGUA GGAUAUAG | 2348 | CUAUAUCC CUGAUGAGGCCGUUAGGCCGAA ACAACCAC | 11766 |
| 953 | UGUAGGAUA UAGGAUUU | 2349 | AAAUCCUA CUGAUGAGGCCGUUAGGCCGAA AUCCUACA | 11767 |
| 955 | UAGGAUAUA GGAUUUAU | 2350 | AUAAAUCC CUGAUGAGGCCGUUAGGCCGAA AUAUCCUA | 11768 |
| 960 | UAUAGGAUU UAUGAUGU | 1626 | ACAUCAUA CUGAUGAGGCCGUUAGGCCGAA AUCCUAUA | 11045 |
| 961 | AUAGGAUUU AUGAUGUG | 1627 | CACAUCAU CUGAUGAGGCCGUUAGGCCGAA AAUCCUAU | 11046 |
| 962 | UAGGAUUUA UGAUGUGA | 2351 | UCACAUCA CUGAUGAGGCCGUUAGGCCGAA AAAUCCUA | 11769 |
| 972 | GAUGUGAUU CUGAGCCC | 2352 | GGGCUCAG CUGAUGAGGCCGUUAGGCCGAA AUCACAUC | 11770 |
| 973 | AUGUGAUUC UGAGCCCC | 2353 | GGGGCUCA CUGAUGAGGCCGUUAGGCCGAA AAUCACAU | 11771 |
| 993 | CAUGAAAUU GAGCUAUC | 2354 | GAUAGCUC CUGAUGAGGCCGUUAGGCCGAA AUUUCAUG | 11772 |
| 999 | AUUGAGCUA UCUGCCGG | 2355 | CCGGCAGA CUGAUGAGGCCGUUAGGCCGAA AGCUCAAU | 11773 |
| 1001 | UGAGCUAUC UGCCGGAG | 2356 | CUCCGGCA CUGAUGAGGCCGUUAGGCCGAA AUAGCUCA | 11774 |
| 1017 | GAAAAACUU GUCUUAAA | 2357 | UUUAAGAC CUGAUGAGGCCGUUAGGCCGAA AGUUUUUC | 11775 |
| 1020 | AAACUUGUC UUAAAUUG | 2358 | CAAUUUAA CUGAUGAGGCCGUUAGGCCGAA ACAAGUUU | 11776 |
| 1022 | ACUUGUCUU AAAUUGUA | 2359 | UACAAUUU CUGAUGAGGCCGUUAGGCCGAA AGACAAGU | 11777 |
| 1023 | CUUGUCUUA AAUUGUAC | 1641 | GUACAAUU CUGAUGAGGCCGUUAGGCCGAA AAGACAAG | 11060 |
| 1027 | UCUUAAAUU GUACAGCG | 2360 | CGCUGUAC CUGAUGAGGCCGUUAGGCCGAA AUUUAAGA | 11778 |
| 1030 | UAAAUUGUA CAGCGAGA | 2361 | UCUCGCUG CUGAUGAGGCCGUUAGGCCGAA ACAAUUUA | 11779 |
| 1047 | ACAGAGCUC AAUGUGGG | 2362 | CCCACAUU CUGAUGAGGCCGUUAGGCCGAA AGCUCUGU | 11780 |
| 1059 | GUGGGGCUU GAUUUCAC | 2363 | GUCAAAUC CUGAUGAGGCCGUUAGGCCGAA AGCCCCAC | 11781 |
| 1063 | GGCUUGAUU UCACCUGG | 2364 | CCAGGUGA CUGAUGAGGCCGUUAGGCCGAA AUCAAGCC | 11782 |
| 1064 | GCUUGALUU CACCUGGC | 2365 | GCCAGGUC CUGAUGAGGCCGUUAGGCCGAA AAUCAAGC | 11783 |
| 1065 | CUUGAUUUC ACCUGGCA | 2366 | UGCCAGGU CUGAUGAGGCCGUUAGGCCGAA AAAUCAAG | 11784 |
| 1076 | CUGGCACUC UCCACCUU | 2367 | AAGGUGGA CUGAUGAGGCCGUUAGGCCGAA AGUOCCAG | 11785 |
| 1078 | GUCACUCUC CACCUUCA | 2368 | UGAAGGUG CUGAUGAGGCCGUUAGGCCGAA AGAGUGCC | 11786 |
| 1084 | CUCCACCUU CAAAGUCU | 2369 | AGACUUUG CUGAUGAGGCCGUUAGGCCGAA AGGUGGAG | 11787 |
| 1085 | UCCACCUUC AAAGUCUC | 2370 | GAGACUUU CUGAUGAGGCCGUUAGGCCGAA AAGGUGGA | 11788 |
| 1091 | UUCAAAGUC UCAUCAUA | 2371 | UAUGAUGA CUGAUGAGGCCGUUAGGCCGAA ACUUUGAA | 11789 |
| 1093 | CAAAGUCUC AUCAUAAG | 2372 | CUUAUGAU CUGAUGAGGCCGUUAGGCCGAA AGACUUUG | 11790 |
| 1096 | AGUCUCAUC AUAAGAAG | 2373 | CUUCUUAU CUGAUGAGGCCGUUAGGCCGAA AUGAGACU | 11791 |
| 1099 | CUCAUCAUA AGAAGAUU | 2374 | AAUCUUCU CUGAUGAGGCCGUUAGGCCGAA AUGAUGAG | 11792 |
| 1107 | AAGAAGAUU GUAAACCG | 2375 | CGGUUUAC CUGAUGAGGCCGUUAGGCCGAA AUCUUCUU | 11793 |
| 1110 | AAGAUUGUA AACCGGGA | 2376 | UCCCGGUU CUGAUGAGGCCGUUAGGCCGAA ACAAUCUU | 11794 |
| 1130 | GAAACCCUU UCCUGGGA | 2377 | UCCCAGGA CUGAUGAGGCCGUUAGGCCGAA AGGGUUUC | 11795 |
| 1131 | AAACCCUUU CCUGGGAC | 2378 | GUCCCAGG CUGAUGAGGCCGUUAGGCCGAA AAGGGUUU | 11796 |
| 1132 | AACCCUUUC CUGGGACU | 2379 | AGUCCCAG CUGAUGAGGCCGUUAGGCCGAA AAAGGGUU | 11797 |
| 1154 | GAAGAUGUU UUUGAGCA | 2380 | UGCUCAAA CUGAUGAGGCCGUUAGGCCGAA ACAUCUUC | 11798 |
| 1155 | AAGAUGUUU UUGAGCAC | 2381 | GUGCUCAA CUGAUGAGGCCGUUAGGCCGAA AACAUCUU | 11799 |
| 1156 | AGAUGUUUU UGAGCACC | 2382 | GGUGCUCA CUGAUGAGGCCGUUAGGCCGAA AAACAUCU | 11800 |
| 1157 | GAUGUUUUU GAGCACCU | 2383 | AGGUGCUC CUGAUGAGGCCGUUAGGCCGAA AAAACAUC | 11801 |
| 1166 | GAGCACCUU GACAAUAG | 2384 | CUAUUGUC CUGAUGAGGCCGUUAGGCCGAA AGGUGCUC | 11802 |
| 1173 | UUGACAAUA GAAAGUGU | 2385 | ACACUUUC CUGAUGAGGCCGUUAGGCCGAA AUUGUCAA | 11803 |
| 1205 | AGGGGAAUA CACCUGUG | 2386 | CACAGGUG CUGAUGAGGCCGUUAGGCCGAA AUUCCCCU | 11804 |
| 1215 | ACCUGUGUA GCGUCCAG | 2387 | CUGGACGC CUGAUGAGGCCGUUAGGCCGAA ACACAGGU | 11805 |
| 1220 | UGUAGCGUC CAGUGGAC | 2388 | GUCCACUG CUGAUGAGGCCGUUAGGCCGAA ACGCUACA | 11806 |
| 1236 | CGGAUGAUC AAGAGAAA | 2389 | UUUCUCUU CUGAUGAGGCCGUUAGGCCGAA AUCAUCCG | 11807 |
| 1246 | AGAGAAAUA GAACAUUU | 2390 | AAAUGUUC CUGAUGAGGCCGUUAGGCCGAA AUUUCUCU | 11808 |
| 1253 | UAGAACAUU UGUCCGAG | 2391 | CUCGGACA CUGAUGAGGCCGUUAGGCCGAA AUGUUCUA | 11809 |
| 1254 | AGAACAUUU GUCCGAGU | 2392 | ACUCGGAC CUGAUGAGGCCGUUAGGCCGAA AAUGUUCU | 11810 |
| 1257 | ACAUUUGUC CGAGUUCA | 2393 | UGAACUCG CUGAUGAGGCCGUUAGGCCGAA ACAAAUGU | 11811 |
| 1263 | GUCCGAGUU CACACAAA | 2394 | UUUGUGUG CUGAUGAGGCCGUUAGGCCGAA ACUCGGAC | 11812 |
| 1264 | UCCGAGUUC ACACAAAG | 2395 | CUUUGUGU CUGAUGAGGCCGUUAGGCCGAA AACUCGGA | 11813 |
| 1276 | CAAAGCCUU UUAUUGCU | 2396 | AGCAAUAA CUGAUGAGGCCGUUAGGCCGAA AGGCUUUG | 11814 |
| 1277 | AAAGCCUUU UAUUGCUU | 2397 | AAGCAAUA CUGAUGAGGCCGUUAGGCCGAA AAGGCUUU | 11815 |
| 1278 | AAGCCUUUU AUUGCUUU | 2398 | AAAGCAAU CUGAUGAGGCCGUUAGGCCGAA AAAGGCUU | 11816 |
| 1279 | AGCCUUUTA UUGCUUUC | 2399 | GAAAGCAA CUGAUGAGGCCGUUAGGCCGAA AAAAGGCU | 11817 |
| 1281 | CCUUUUAUU GCUUUCGG | 2400 | CCGAAAGC CUGAUGAGGCCGUUAGGCCGAA AUAAAAGG | 11818 |
| 1285 | UUAUUGCUU UCGGUAGU | 2401 | ACUACCGA CUGAUGAGGCCGUUAGGCCGAA AGCAAUAA | 11819 |
| 1286 | UAUUGCUUU CGGUAGUG | 2402 | CACUACCG CUGAUGAGGCCGUUAGGCCGAA AAGCAAUA | 11820 |
| 1287 | AUUGCUUUC GGUAGUGG | 2403 | CCACUACC CUGAUGAGGCCGUUAGGCCGAA AAAGCAAU | 11821 |
| 1291 | CUUUCGGUA GUOGGAUG | 2404 | CAUCCCAC CUGAUGAGGCCGUUAGGCCGAA ACCGAAAG | 11822 |
| 1304 | GAUGAAAUC UUUGGUGG | 2405 | CCACCAAA CUGAUGAGGCCGUUAGGCCGAA AUUUCAUC | 11823 |
| 1306 | UGAAAUCUU UGGUGGAA | 2406 | UUCCACCA CUGAUGAGGCCGUUAGGCCGAA AGAUUUCA | 11824 |
| 1307 | GAAAUCUUU GGUGGAAG | 2407 | CUUCCACC CUGAUGAGGCCGUUAGGCCGAA AAGAUUUC | 11825 |
| 1330 | UGGGCAGUC AAGUCCGA | 2408 | UCGGACUU CUGAUGAGGCCGUUAGGCCGAA ACUGCCCA | 11826 |
| 1335 | AGUCAAGUC CGAAUCCC | 2409 | GGGAUUCG CUGAUGAGGCCGUUAGGCCGAA ACUUGACU | 11827 |
| 1341 | GUCCGAAUC CCUGUGAA | 2410 | UUCACAGG CUGAUGAGGCCGUUAGGCCGAA AUUCGGAC | 11828 |
| 1352 | UGUGAACUA UCUCAGUU | 2411 | AACUGAGA CUGAUGAGGCCGUUAGGCCGAA ACUUCACA | 11829 |
| 1354 | UGAAGUAUC UCAGUUAC | 2412 | GUAACUGA CUGAUGAGGCCGUUAGGCCGAA AUACUUCA | 11830 |
| 1356 | AAGUAUCUC AGUUACCC | 2413 | GGGUAACU CUGAUGAGGCCGUUAGGCCGAA AGAUACUU | 11831 |
| 1360 | AUCUCAGUU ACCCAGCU | 2414 | AGCUGGGU CUGAUGAGGCCGUUAGGCCGAA ACUGAGAU | 11832 |
| 1361 | UCUCAGUUA CCCAGCUC | 2415 | GAGCUGGG CUGAUGAGGCCGUUAGGCCGAA AACUGAGA | 11833 |
| 1369 | ACCCAGCUC CUGAUAUC | 2416 | GAUAUCAG CUGAUCAGGCCCUUAGGCCGAA AGCUGGGU | 11834 |
| 1375 | CUCCUGAUA UCAAAUGG | 2417 | CCAUUUGA CUGAUGAGGCCGUUAGGCCGAA AUCAGGAC | 11835 |
| 1377 | CCUGAUAUC AAAUGGUA | 2418 | UACCAUUU CUGAUGAGGCCGUUAGGCCGAA AUAUCAGG | 11836 |
| 1385 | CAAAUGGUA CAGAAAUG | 2419 | CAUUUCUG CUGAUGAGGCCGUUAGGCCCAA ACCADUUG | 11837 |
| 1404 | AGGCCCAUU GAGUCCPA | 2420 | UUGGACUC CUGAUGAGGCCGUUAGGCCGAA AUGGGCCU | 11838 |
| 1409 | CAUUGAGUC CAACUACA | 2421 | UGUAGUUG CUGAUGAGGCCGUUAGGCCGAA ACUCAAUG | 11839 |
| 1415 | GUCCAACUA CACAAUGA | 2422 | UCAUUGUG CUGAUGAGGCCGUUAGGCCGAA AGUUGGAC | 11840 |
| 1425 | ACAAUGAUU GUUGGCGA | 2423 | UCGCCAAC CUGAUGAGGCCGUUAGGCCGAA AUCAUUGU | 11841 |
| 1428 | AUGAUUGUU GGCGAUGA | 2424 | UCAUCGCC CUGAUGAGGCCGUUAGGCCGAA ACAAUCAU | 11842 |
| 1440 | GAUGAACUC ACCAUCAU | 2425 | AUGAUGGU CUGAUGAGGCCGUUAGGCCGAA AGUUCAUC | 11843 |
| 1446 | CUCACCAUC AUGGAAGU | 2426 | ACUUCCAU CUGAUGAGGCCGUUAGGCCGAA AUGGUGAG | 11844 |
| 1478 | AGGAAACUA CACGGUCA | 2427 | UGACCGUG CUGAUGAGGCCGUUAGGCCGAA AGUUUCCU | 11845 |
| 1485 | UACACGGUC AUCCUCAC | 2428 | GUGAGGAU CUGAUGAGGCCGUUAGGCCGAA ACCGUGUA | 11846 |
| 1488 | ACGGUCAUC CUCACCAA | 2429 | UUGGUGAG CUGAUGAGGCCGUUAGGCCGAA AUGACCGU | 11847 |
| 1491 | GUCAUCCUC ACCAACCC | 2430 | GGGUUGGU CUGAUGAGGCCGUUAGGCCGAA AGGAUGAC | 11848 |
| 1503 | AACCCCAUU UCAAUGGA | 2431 | UCCAUUGA CUGAUGAGGCCGUUAGGCCGAA AUGGGGUU | 11849 |
| 1504 | ACCCCAUUU CAAUGGAG | 2432 | CUCCAUUG CUGAUGAGGCCGUUAGGCCGAA AAUGGGGU | 11850 |
| 1505 | CCCCAUUUC AAUGGAGA | 2433 | UCUCCAUU CUGAUGAGGCCGUUAGGCCGAA AAAUGGGG | 11851 |
| 1530 | CACAUGGUC UCUCUGGU | 2434 | ACCAGAGA CUGAUGAGGCCGUUAGGCCGAA ACCAUGUG | 11852 |
| 1532 | CAUGGUCUC UCUGGUUG | 2435 | CAACCAGA CUGAUGAGGCCGUUAGGCCGAA AGACCAUG | 11853 |
| 1534 | UGGUCUCUC UGGUUGUG | 1714 | CACAACCA CUGAUGAGGCCGUUAGGCCGAA AGAGACCA | 11133 |
| 1539 | UCUCUGGUU GUGAAUGU | 2436 | ACAUUCAC CUGAUGAGGCCGUUAGGCCGAA ACCAGAGA | 11854 |
| 1548 | GUGAAUGUC CCACCCCA | 2437 | UGGGGUGG CUGAUGAGGCCGUUAGGCCGAA ACAUUCAC | 11855 |
| 1560 | CCCCAGAUC GGUGAGAA | 2438 | UUCUCACC CUGAUGAGGCCGUUAGGCCGAA AUCUGGGG | 11137 |
| 1574 | GAAAGCCUU GAUCUCGC | 2439 | GCGAGAUC CUGAUGAGGCCGUUAGGCCGAA AGGCUUUC | 11856 |
| 1578 | GCCUUGAUC UCGCCUAU | 2440 | AUAGGCGA CUGAUGAGGCCGUUAGGCCGAA AUCAAGGC | 11857 |
| 1580 | CUUGAUCUC GCCUAUGG | 2441 | CCAUAGGC CUGAUGAGGCCGUUAGGCCGAA AGAUCAAG | 11858 |
| 1585 | UCUCGCCUA UGGAUUCC | 2442 | GGAAUCCA CUGAUGAGGCCGUUAGGCCGAA AGGCGAGA | 11859 |
| 1591 | CUAUGGAUU CCUACCAG | 2443 | CUGGUAGG CUGAUGAGGCCGUUAGGCCGAA AUCCAUAG | 11860 |
| 1592 | UAUGGAUUC CUACCAGU | 2444 | ACUGGUAG CUGAUGAGGCCGUUAGGCCGAA AAUCCAUA | 11861 |
| 1595 | GGAUUCCUA CCAGUAUG | 2445 | CAUACUGG CUGAUGAGGCCGUUAGGCCGAA AGGAAUCC | 11862 |
| 1601 | CUACCAGUA UGGGACCA | 2446 | UGGUCCCA CUGAUGAGGCCGUUAGGCCGAA ACUGGUAG | 11863 |
| 1619 | GCAGACAUU GACAUGCA | 2447 | UGCAUGUC CUGAUGAGGCCGUUAGGCCGAA AUGUCUGC | 11864 |
| 1632 | UGCACAGUC UACGCCAA | 2448 | UUGGCGUA CUGAUGAGGCCGUUAGGCCGAA ACUGUGCA | 11865 |
| 1634 | CACAGUCUA CGCCAACC | 2449 | GGUUGGCG CUGAUGAGGCCGUUAGGCCGAA AGACUGUG | 11866 |
| 1645 | CCAACCCUC CCCUGCAC | 2450 | GUGCAGGG CUGAUGAGGCCGUUAGGCCGAA AGGGUUGG | 11867 |
| 1659 | CACCACAUC CAGUGGUA | 2451 | UACCACUG CUGAUGAGGCCGUUAGGCCGAA AUGUGGUG | 11868 |
| 1667 | CCAGUGGUA CUGGCAGC | 2452 | GCUGCCAG CUGAUGAGGCCGUUAGGCCGAA ACCACUGG | 11869 |
| 1677 | UGGCAGCUA GAAGAAGC | 2453 | GCUUCUUC CUGAUGAGGCCGUUAGGCCGAA AGCUGCCA | 11870 |
| 1691 | AGCCUGCUC CUACAGAC | 2454 | GUCUGUAG CUGAUGAGGCCGUUAGGCCGAA AGCAGGCU | 11871 |
| 1694 | CUGCUCCUA CAGACCCG | 2455 | CGGGUCUG CUGAUGAGGCCGUUAGGCCGAA AGGAGCAG | 11872 |
| 1718 | AAGCCCGUA UGCUUGUA | 2456 | UACAAGCA CUGAUGAGGCCGUUAGGCCGAA ACGGGCUU | 11873 |
| 1723 | CGUAUGCUU GUAAAGAA | 2457 | UUCUUUAC CUGAUGAGGCCGUUAGGCCGAA AGCAUACG | 11874 |
| 1726 | AUGCUUGUA AAGAAUGG | 2458 | CCAUUCUU CUGAUGAGGCCGUUAGGCCGAA ACAAGCAU | 11875 |
| 1750 | UGGAGGAUU UCCAGGGG | 2459 | CCCCUGGA CUGAUGAGGCCGUUAGGCCGAA AUCCUCCA | 11876 |
| 1751 | GGAGGAUUU CCAGGGGG | 2460 | CCCCCUGG CUGAUGAGGCCGUUAGGCCGAA AAUCCUCC | 11877 |
| 1752 | GAGGAUUUC CAGGGGGG | 2461 | CCCCCCUG CUGAUGAGGCCGUUAGGCCGAA AAAUCCUC | 11878 |
| 1770 | AACAAGAUC GAAGUCAC | 2462 | UUGACUUC CUGAUGAGGCCGUUAGGCCGAA AUCUUGUU | 11879 |
| 1776 | AUCGAAGUC ACCAAAAA | 2463 | UUUUUGGU CUGAUGAGGCCGUUAGGCCGAA ACUUCGAU | 11880 |
| 1790 | AAACCAAUA UGCCCUGA | 2464 | UCAGGGCA CUGAUGAGGCCGUUAGGCCGAA AUUGGUUU | 11881 |
| 1800 | GCCCUGAUU GAAGGAAA | 2465 | UUUCCUUC CUGAUGAGGCCGUUAGGCCGAA AUCAGGGC | 11882 |
| 1821 | AAAACUGUA AGUACGCU | 2466 | AGCGUACU CUGAUGAGCCCGUUAGGCCGAA ACAGUUUU | 11883 |
| 1825 | CUGUAAGUA COCUGGUC | 2467 | GACCAGCG CUGAUGAGGCCGUUAGGCCGAA ACUUACAG | 11884 |
| 1833 | ACGCUGGUC AUCCAAGC | 2468 | GCUUGGAU CUGAUGAGGCCGUUAGGCCGAA ACCAGCGU | 11885 |
| 1836 | CUGGUCAUC CAAGCUGC | 2469 | GCAGCUUG CUGAUGAGGCCGUUAGGCCGAA AUGACCAG | 11886 |
| 1853 | CAACGUGUC AGCGUUGU | 2470 | ACAACGCU CUGAUGAGGCCGUUAGGCCGAA ACACGUUG | 11887 |
| 1859 | GUCAGCGUU GUACAAAU | 2471 | AUUUGUAC CUGAUGAGGCCGUUAGGCCGAA ACOCUGAC | 11888 |
| 1862 | AGCGUUGUA CAAAUGUG | 2472 | CACAUUUG CUGAUGAGGCCGUUAGGCCGAA ACAACGCU | 11889 |
| 1878 | GAAGCCAUC AACAAAGC | 2473 | GCUUUGUU CUGAUGAGGCCGUUAGGCCGAA AUGGCUUC | 11890 |
| 1905 | GAGAGGGUC AUCUCCUU | 2474 | AAGGAGAU CUGAUGAGGCCGUUAGGCCGAA ACCCUCUC | 11891 |
| 1908 | AGGGUCAUC UCCUUCCA | 2475 | UGGAAGGA CUGAUGAGGCCGUUAGGCCGAA AUGACCCU | 11892 |
| 1910 | GGUCAUCUC CUUCCAUG | 2476 | CAUGGAAG CUGAUGAGGCCGUUAGGCCGAA AGAUGACC | 11893 |
| 1913 | CAUCUCCUU CCAUGUGA | 2477 | UCACAUGG CUGAUGAGGCCGUUAGGCCGAA AGGAGAUG | 11894 |
| 1914 | AUCUCCUUC CAUGUGAU | 2478 | AUCACAUG CUGAUGAGGCCGUUAGGCCGAA AAGGAGAU | 11895 |
| 1923 | CAUGUGAUC AGGGGUCC | 2479 | GGACCCCU CUGAUGAGGCCGUUAGGCCGAA AUCACAUG | 11896 |
| 1930 | UCAGGGGUC CUGAAAUU | 2480 | AAUUUCAG CUGAUGAGGCCGUUAGGCCGAA ACCCCUGA | 11897 |
| 1938 | CCUGAAAUU ACUGUGCA | 2481 | UGCACAGU CUGAUGAGGCCGUUAGGCCGAA AUUUCAGG | 11898 |
| 1939 | CUGAAAUUA CUGUGCAA | 2482 | UUGCACAG CUGAUGAGGCCGUUAGGCCGAA AAUUUCAG | 11899 |
| 1982 | GAGUGUGUC CCUGUUGU | 2483 | ACAACAGG CUGAUGAGGCCGUUAGGCCGAA ACACACUC | 11900 |
| 1988 | GUCCCUGUU GUGCACUG | 2484 | CAGUGCAC CUGAUGAGGCCGUUAGGCCGAA ACAGGGAC | 11901 |
| 2008 | ACAGAAAUA CGUUUGAG | 2485 | CUCAAACG CUGAUGAGGCCGUUAGGCCGAA AUUUCUGU | 11902 |
| 2012 | AAAUACGUU UGAGAACC | 2486 | GGUUCUCA CUGAUGAGGCCGUUAGGCCGAA ACGUAUUU | 11903 |
| 2013 | AAUACGUUU GAGAACCU | 2487 | AGGUUCUC CUGAUGAGGCCGUUAGGCCGAA AACGUAUU | 11904 |
| 2022 | GAGAACCUC ACGUGGUA | 2488 | UACCACGU CUGAUGAGGCCGUUAGGCCGAA AGGUUCUC | 11905 |
| 2030 | CACGUGGUA CAAGCUUG | 2489 | CAAGCUUG CUGAUGAGGCCGUUAGGCCGAA ACCACGUG | 11906 |
| 2037 | UACAAGCUU GGCUCACA | 2490 | UGUGAGCC CUGAUGAGGCCGUUAGGCCGAA AGCUUGUA | 11907 |
| 2042 | GCUUGGCUC ACAGGCAA | 2491 | UUGCCUGU CUGAUGAGGCCGUUAGGCCGAA AGCCAAGC | 11908 |
| 2054 | GGCAACAUC GGUCCACA | 2492 | UGUGGACC CUGAUGAGGCCGUUAGGCCGAA AUGUUGCC | 11909 |
| 2058 | ACAUCGGUC CACAUGGG | 2493 | CCCAUGUG CUGAUGAGGCCGUUAGGCCGAA ACCGAUGU | 11910 |
| 2072 | GGGCGAAUC ACUCACAC | 2494 | GUGUGAGU CUGAUGAGGCCGUUAGGCCGAA AUUCGCCC | 11911 |
| 2076 | GAAUCACUC ACACCAGU | 2495 | ACUGGUGU CUGAUGAGGCCGUUAGGCCGAA AGUGAUUC | 11912 |
| 2085 | ACACCAGUU UGCAAGAA | 2496 | UUCUUGCA CUGAUGAGGCCGUUAGGCCGAA ACUGGUGU | 11913 |
| 2086 | CACCAGUUU GCAAGAAC | 2497 | GUUCUUGC CUGAUGAGGCCGUUAGGCCGAA AACUGGUG | 11914 |
| 2096 | CAAGAACUU GGAUGCUC | 2498 | GAGCAUCC CUGAUGAGGCCGUUAGGCCGAA AGUUCUUG | 11915 |
| 2104 | UGGAUGCUC UUUGGAAA | 2499 | UUUCCAAA CUGAUGAGGCCGUUAGGCCGAA AGCAUCCA | 11916 |
| 2106 | GAUGCUCUU UGGAAACU | 2500 | AGUUUCCA CUGAUGAGGCCGUUAGGCCGAA AGAGCAUC | 11917 |
| 2107 | AUGCUCUUU GGAAACUG | 2501 | CAGUUUCC CUGAUGAGGCCGUUAGGCCGAA AAGAGCAU | 11918 |
| 2129 | CACCAUGUU UUCUAACA | 2502 | UGUUAGAA CUGAUGAGGCCGUUAGGCCGAA ACAUGGUG | 11919 |
| 2130 | ACCAUGUUU UCUAACAG | 2503 | CUGUUAGA CUGAUGAGGCCGUUAGGCCGAA AACAUGGU | 11920 |
| 2131 | CCAUGUUUU CUAACAGC | 2504 | GCUGUUAG CUGAUGAGGCCGUUAGGCCGAA AAACAUGG | 11921 |
| 2132 | CAUGUUUUC UAACAGCA | 2505 | UGCUGUUA CUGAUGAGGCCGUUAGGCCGAA AAAACAUG | 11922 |
| 2134 | UGUUUUCUA ACAGCACA | 2506 | UGUGCUGU CUGAUGAGGCCGUUAGGCCGAA AGAAAACA | 11923 |
| 2151 | AAUGACAUC UUGAUUGU | 2507 | ACAAUCAA CUGAUGAGGCCGUUAGGCCGAA AUGUCAUU | 11924 |
| 2153 | UGACAUCUU GAUUGUGG | 2508 | CCACAAUC CUGAUGAGGCCGUUAGGCCGAA AGAUGUCA | 11925 |
| 2157 | AUCUUGAUU GUGGCAUU | 2509 | AAUGCCAC CUGAUGAGGCCGUUAGGCCGAA AUCAAGAU | 11926 |
| 2165 | UGUGGCAUU UCAGAAUG | 2510 | CAUUCUGA CUGAUGAGGCCGUUAGGCCGAA AUGCCACA | 11927 |
| 2166 | GUGGCAUUU CAGAAUGC | 2511 | GCAUUCUG CUGAUGAGGCCGUUAGGCCGAA AAUGCCAC | 11928 |
| 2167 | UGGCAUUUC AGAAUGCC | 2512 | GGCAUUCU CUGAUGAGGCCGUUAGGCCGAA AAAUGCCA | 11929 |
| 2177 | GAAUGCCUC UCUGCAGG | 2513 | CCUGCAGA CUGAUGAGGCCGUUAGGCCGAA AGGCAUUC | 11930 |
| 2179 | AUGCCUCUC UGCAGGAC | 2514 | GUCCUGCA CUGAUGAGGCCGUUACCCCGAA ACAGGCAU | 11931 |
| 2198 | AGGCGACUA UGUUUGCU | 2515 | AGCAAACA CUGAUGAGGCCGUUACCCCGAA AGUCGCCU | 11932 |
| 2202 | GACUAUGUU UGCUCUGC | 2518 | GCAGAGCA CUGAUGAGGCCGUUAGGCCGAA ACAUAGUC | 11933 |
| 2203 | ACUAUGUUU GCUCUGCU | 2517 | AGCAGAGC CUGAUGAGGCCGUUAGGCCGAA AACAUAGU | 11934 |
| 2207 | UGUUUGCUC UGCUCAAG | 2518 | CUUGAGCA CUGAUGAGGCCGUUAGGCCGAA AGCAAACA | 11935 |
| 2212 | GCUCUGCUC AAGAUAAG | 2519 | CUUAUCUU CUGAUGAGGCCGUUAGGCCGAA AGCAGAGC | 11936 |
| 2218 | CUCAAGAUA AGAAGACC | 2520 | GGUCUUCU CUGAUGAGGCCGUUAGGCCGAA AUCUUGAG | 11937 |
| 2239 | AAAGACAUU GCCUGGUC | 2521 | GACCAGGC CUGAUGAGGCCGUUAGGCCGAA AUGUCUUU | 11938 |
| 2247 | UGCCUGGUC AAACAGCU | 2522 | AGCUGUUU CUGAUGAGGCCGUUAGGCCGAA ACCAGGCA | 11939 |
| 2256 | AAACAGCUC AUCAUCCU | 2523 | AGGAUGAU CUGAUGAGGCCGUUAGGCCGAA AGCUGUUU | 11940 |
| 2259 | CAGCUCAUC AUCCUAGA | 2524 | UCUAGGAU CUGAUGAGGCCGUUAGGCCGAA AUGAGCUG | 11941 |
| 2262 | CUCAUCAUC CUAGAGCG | 2525 | CGCUCUAG CUGAUGAGGCCGUUAGGCCGAA AUGAUGAG | 11942 |
| 2265 | AUCAUCCUA GAGCGCAU | 2526 | AUGCGCUC CUGAUGAGGCCGUUAGGCCGAA AGGAUGAG | 11943 |
| 2286 | CCCAUGAUC ACCGGAAA | 2527 | UUUCCGGU CUGAUGAGGCCGUUAGGCCGAA AUCAUGGG | 11944 |
| 2296 | CCGGAAAUC UGGAGAAU | 2528 | AUUCUCCA CUGAUGAGGCCGUUAGGCCGAA AUUUCCGG | 11945 |
| 2305 | UGGAGAAUC AGACAACA | 2529 | UGUUGUCU CUGAUGAGGCCGUUAGGCCGAA AGUCUCCA | 11946 |
| 2319 | ACAACCAUU GGCGAGAC | 2530 | GUCUCGCC CUGAUGAGGCCGUUAGGCCGAA AUGGUUGU | 11947 |
| 2331 | GAGACCAUU GAAGUGAC | 2531 | GUCACUUC CUGAUGAGGCCGUUAGGCCGAA AUGGUCUC | 11948 |
| 2341 | AAGUGACUU GCCCAGCA | 2532 | UGCUGGGC CUGAUGAGGCCGUUAGGCCGAA AGUCACUU | 11949 |
| 2351 | CCCAGCAUC UGGAAAUC | 2533 | GAUUUCCA CUGAUGAGGCCGUUAGGCCGAA AUGCUGGG | 11950 |
| 2359 | CUGGAAAUC CUACCCCA | 2534 | UGGGGUAG CUGAUGAGGCCGUUAGGCCGAA AUUUCCAG | 11951 |
| 2362 | GAAAUCCUA CCCCACAC | 2535 | GUGUGGGG CUGAUGAGGCCGUUAGGCCGAA AGGAUUUC | 11952 |
| 2373 | CCACACAUU ACAUGGUU | 2536 | AACCAUGU CUGAUGAGGCCGUUAGGCCGAA AUGUGUGG | 11953 |
| 2374 | CACACAUUA CAUGGUUC | 2537 | GAACCAUG CUGAUGAGGCCGUUAGGCCGAA AAUGUGUG | 11954 |
| 2381 | UACAUGGUU CAAAGACA | 2538 | UGUCUUUG CUGAUGAGGCCGUUAGGCCGAA ACCAUGUA | 11955 |
| 2382 | ACAUGGUUC AAAGACAA | 2539 | UUGUCUUU CUGAUGAGGCCGUUAGGCCGAA AACCAUGU | 11956 |
| 2403 | ACCCUGGUA GAAGAUUC | 2540 | GAAUCUUC CUGAUGAGGCCGUUAGGCCGAA ACCAGGGU | 11957 |
| 2410 | UAGAAGAUU CAGGCAUU | 2541 | AAUGCCUG CUGAUGAGGCCGUUAGGCCGAA AUCUUCUA | 11958 |
| 2411 | AGAAGAUUC AGGCAUUG | 2542 | CAAUGCCU CUGAUGAGGCCGUUAGGCCGAA AAUCUUCU | 11959 |
| 2418 | UCAGGCAUU GUACUGAG | 2543 | CUCAGUAC CUGAUGAGGCCGUUAGGCCGAA AUGCCUGA | 11960 |
| 2421 | GGCAUUGUA CUGAGAGA | 2544 | UCUCUCAG CUGAUGAGGCCGUUAGGCCGAA ACAAUGCC | 11961 |
| 2449 | ACCUGACUA UCCGCAGG | 2545 | CCUGCGGA CUGAUGAGGCCGUUAGGCCGAA AGUCAGGU | 11962 |
| 2451 | CUGACUAUC CGCAGGGU | 2546 | ACCCUGCG CUGAUGAGGCCGUUAGGCCGAA AUAGUCAG | 11963 |
| 2481 | GGAGGCCUC UACACCUG | 2547 | CAGGUGUA CUGAUGAGGCCGUUAGGCCGAA AGGCCUCC | 11964 |
| 2483 | AGGCCUCUA CACCUGCC | 1847 | GGCAGGUG CUGAUGAGGCCGUUAGGCCGAA AGAGGCCU | 11266 |
| 2505 | UGCAAUGUC CUUGGCUG | 2548 | CAGCCAAG CUGAUGAGGCCGUUAGGCCGAA ACAUUGCA | 11965 |
| 2508 | AAUGUCCUU GGCUGUGC | 2549 | GCACAGCC CUGAUGAGGCCGUUAGGCCGAA AGGACAUU | 11966 |
| 2532 | GAGACGCUC UUCAUAAU | 2550 | AUUAUGAA CUGAUGAGGCCGUUAGGCCGAA AGCGUCUC | 11967 |
| 2534 | GACGCUCUU CAUAAUAG | 2551 | CUAUUAUG CUGAUGAGGCCGUUAGGCCGAA AGAGCGUC | 11968 |
| 2535 | ACGCUCUUC AUAAUAGA | 2552 | UCUAUUAU CUGAUGAGGCCGUUAGGCCGAA AAGAGCGU | 11969 |
| 2538 | CUCUUCAUA AUAGAAGG | 2553 | CCUUCUAU CUGAUGAGGCCGUUAGGCCGAA AUGAAGAG | 11970 |
| 2541 | UUCAUAAUA GAAGGUGC | 1857 | GCACCUUC CUGAUGAGGCCGUUAGGCCGAA AUUAUGAA | 11276 |
| 2567 | GACCAACUU GGAAGUCA | 2554 | UGACUUCC CUGAUGAGGCCGUUAGGCCGAA AGUUGGUC | 11971 |
| 2574 | UUGGAAGUC AUUAUCCU | 2555 | AGGAUAAU CUGAUGAGGCCGUUAGGCCGAA ACUUCCAA | 11972 |
| 2577 | GAAGUCAUU AUCCUCGU | 2556 | ACGAGGAU CUGAUGAGGCCGUUAGGCCGAA AUGACUUC | 11973 |
| 2578 | AAGUCAUUA UCCUCGUC | 2557 | GACGAGGA CUGAUGAGGCCGUUAGGCCGAA AAUGACUU | 11974 |
| 2580 | GUCAUUAUC CUCGUCGG | 2558 | CCGACGAG CUGAUGAGGCCGUUAGGCCGAA AUAAUGAC | 11975 |
| 2583 | AUUAUCCUC GUCGGCAC | 2559 | GUGCCGAC CUGAUGAGGCCGUUAGGCCGAA AGGAUAAU | 11976 |
| 2586 | AUCCUCCUC GGCACUGC | 2560 | GCAGUGCC CUGAUGAGGCCGUUAGGCCGAA ACGAGGAU | 11977 |
| 2601 | GCAGUGAUU GCCAUGUU | 2561 | AACAUGGC CUGAUGAGGCCGUUAGGCCGAA AUCACUGC | 11978 |
| 2609 | UGCCAUGUU CUUCUGGC | 1867 | GCCAGAAG CUGAUGAGGCCGUUAGGCCGAA ACAUGGCA | 11286 |
| 2610 | GCCAUGUUC UUCUGGCU | 1868 | AGCCAGAA CUGAUGAGGCCGUUAGGCCGAA AACAUGGC | 11287 |
| 2612 | CAUGUUCUU CUGGCUCC | 2562 | GGAGCCAG CUGAUGAGGCCGUUAGGCCGAA AGAACAUG | 11979 |
| 2613 | AUGUUCUUC UGGCUCCU | 2563 | AGGAGCCA CUGAUGAGGCCGUUAGGCCGAA AAGAACAU | 11980 |
| 2619 | UUCUGGCUC CUUCUUGU | 2564 | ACAAGAAG CUCAUGAGGCCGUUAGGCCGAA AGCCAGAA | 11290 |
| 2622 | UGGCUCCUU CUUGUCAU | 2565 | AUGACAAG CUGAUGAGGCCGUUAGGCCGAA AGGAGCCA | 11981 |
| 2623 | GGCUCCUUC UUCUCAUU | 2566 | AAUGACAA CUGAUGAGGCCGUUACGCCGAA AAGGACCC | 11982 |
| 2625 | CUCCUUCUU GUCAUUGU | 2567 | ACAAUGAC CUGAUGAGGCCGUUAGGCCGAA AGAAGCAG | 11983 |
| 2628 | CUUCUUGUC AUUGUCCU | 2568 | AGGACAAU CUGAUGAGGCCGUUAGGCCGAA ACAAGAAG | 11984 |
| 2631 | CUUGUCAUU GUCCUACG | 2569 | CGUAGGAC CUGAUGAGGCCGUUAGCCCGAA AUGACAAG | 11985 |
| 2634 | GUCAUUGUC CUACGUAC | 2570 | GUCCGUAG CUGAUGAGGCCGUUAGCCCGAA ACAAUGAC | 11986 |
| 2637 | AUUGUCCUA CGGACCGU | 2571 | ACGGUCCG CUGAUGAGGCCGUUAGGCCGAA AGGACAAU | 11987 |
| 2646 | CGGACCGUU AAGCGGGC | 2572 | GCCCGCUU CUGAUGAGGCCGUUAGGCCGAA ACGGUCCG | 11988 |
| 2647 | GGACCCUUA AGCGGGCC | 1880 | GGCCCGCU CUGAUGAGGCCGUUAGGCCGAA AACGGUCC | 11299 |
| 2681 | GACAGGCUA CUUGUCUA | 2573 | UAGACAAG CUGAUGAGGCCGUUAGGCCGAA AGCCUGUC | 11989 |
| 2684 | AGGCUACUU GUCUAUUG | 2574 | CAAUAGAC CUGAUGAGGCCGUUAGGCCGAA AGUAGCCU | 11990 |
| 2687 | CUACUUGUC UAUUGUCA | 2575 | UGACAAUA CUGAUGAGGCCGUUAGGCCGAA ACAAGUAG | 11991 |
| 2689 | ACUUGUCUA GUGUCAUG | 2576 | CAUGACAA CUGAUGAGGCCGUUAGGCCGAA AGACAAGU | 11992 |
| 2691 | UUGUCUAUU GUCAUGGA | 2577 | UCCAUGAC CUGAUGAGGCCGUUAGGCCGAA AUAGACAA | 11993 |
| 2694 | UCUAUUGUC AUGGAUCC | 2578 | GGAUCCAU CUGAUGAGGCCGUUAGGCCGAA ACAAUAGA | 11994 |
| 2701 | UCAUGGAUC CAGAUGAA | 1886 | UUCAUCUG CUGAUGAGGCCGUUAGGCCGAA AUCCAUGA | 11305 |
| 2711 | AGAUGAAUU GCCCUUGG | 2579 | CCAAGGGC CUGAUGAGGCCGUUAGGCCGAA AUUCAUCU | 11995 |
| 2717 | AUUGCCCUU GGAUGAGC | 2580 | GCUCAUCC CUGAUGAGGCCGUUAGGCCGAA AGGGCAAU | 11996 |
| 2738 | UGAACGCUU GCCUUAUG | 2581 | CAUAAGGC CUGAUGAGGCCGUUAGGCCGAA AGCGUUCA | 11997 |
| 2743 | GCUUGCCUU AUGAUGCC | 2582 | GGCAUCAU CUGAUGAGGCCGUUAGGCCGAA AGGCAAGC | 11998 |
| 2744 | CUUGCCUUA UGAUGCCA | 2583 | UGGCAUCA CUGAUGAGGCCGUUAGGCCGAA AAGGCAAG | 11999 |
| 2765 | GUGGGAAUU CCCCAGGG | 2584 | CCCUGGGG CUGAUGAGGCCGUUAGGCCGAA AUUCCCAC | 12000 |
| 2766 | UGGGAAUUC CCCAGGGA | 2585 | UCCCUGGG CUGAUGAGGCCGUUAGGCCGAA AAUUCCCA | 12001 |
| 2787 | CUGAAACUA GGAAAACC | 2586 | GGUUUUCC CUGAUGAGGCCGUUAGGCCGAA AGUUUCAG | 12002 |
| 2797 | GAAAACCUC UUGGCCGC | 2587 | GCGGCCAA CUGAUGAGGCCGUUAGGCCGAA AGGUUUUC | 12003 |
| 2799 | AAACCUCUU GGCCGCGG | 2588 | CCGCGGCC CUGAUGAGGCCGUUAGGCCGAA AGAGGUUU | 12004 |
| 2813 | CGGUGCCUU CGGCCAAG | 2589 | CUUGGCCG CUGAUGAGGCCGUUAGGCCGAA AGGCACCG | 12005 |
| 2814 | GGUGCCUUC GGCCAAGU | 2590 | ACUUGGCC CUGAUGAGGCCGUUAGGCCGAA AAGGCACC | 12006 |
| 2826 | CAAGUGAUU GAGGCAGA | 2591 | UCUGCCUC CUGAUGAGGCCGUUAGGCCGAA AUCACUUG | 12007 |
| 2839 | CAGACGCUU UUGGAAUU | 2592 | AAUUCCAA CUGAUGAGGCCGUUAGGCCGAA AGCGUCUG | 12008 |
| 2840 | AGACGCUUU UGGAAUUG | 2593 | CAAUUCCA CUGAUGAGGCCGUUAGGCCGAA AAGCGUCU | 12009 |
| 2841 | GACGCUUUU GGAAUUGA | 2594 | UCAAUUCC CUGAUGAGGCCGUUAGGCCGAA AAAGCGUC | 12010 |
| 2847 | UUUGGAAUU GACAAGAC | 1903 | GUCUUGUC CUGAUGAGGCCGUUAGGCCGAA AUUCCAAA | 11322 |
| 2863 | CAGCGACUU GCAAAACA | 2595 | UGUUUUGC CUGAUGAGGCCGUUAGGCCGAA AGUCGCUG | 12011 |
| 2874 | AAAACAGUA GCCGUCAA | 2596 | UUGACGGC CUGAUGAGGCCGUUAGGCCGAA ACUGUUUU | 12012 |
| 2880 | GUAGCCGUC AAGAUGUU | 2597 | AACAUCUU CUGAUGAGGCCGUUAGGCCGAA ACGGCUAC | 12013 |
| 2888 | CAAGAUGUU GAAAGAAG | 2598 | CUUCUUUC CUGAUGAGGCCGUUAGGCCGAA ACAUCUUG | 12014 |
| 2917 | GCGAGCAUC GAGCCCUC | 2599 | GAGGGCUC CUGAUGAGGCCGUUAGGCCGAA AUGCUCGC | 12015 |
| 2925 | CGAGCCCUC AUGUCUGA | 2600 | UCAGACAU CUGAUGAGGCCGUUAGGCCGAA AGGGCUCG | 12016 |
| 2930 | CCUCAUGUC UGAACUCA | 2601 | UGAGUUCA CUGAUGAGGCCGUUAGGCCGAA ACAUGAGG | 12017 |
| 2937 | UCUGAACUC AAGAUCCU | 1912 | AGGAUCUU CUGAUGAGGCCGUUAGGCCGAA AGUUCAGA | 11331 |
| 2943 | CUCAAGAUC CUCAUCCA | 2602 | UGGAUGAG CUGAUGAGGCCGUUAGGCCGAA AUCUUGAG | 12018 |
| 2946 | AAGAUCCUC AUCCACAU | 2603 | AUGUGGAU CUGAUGAGGCCGUUAGGCCGAA AGGAUCUU | 12019 |
| 2949 | AUCCUCAUC CACAUUGG | 2604 | CCAAUGUG CUGAUGAGGCCGUUAGGCCGAA AUGAGGAU | 12020 |
| 2955 | AUCCACAUU GGUCACCA | 2605 | UGGUGACC CUGAUGAGGCCGUUAGGCCGAA AUGUGGAU | 12021 |
| 2959 | ACAUUGGUC ACCAUCUC | 2606 | GAGAUGGU CUGAUGAGGCCGUUAGGCCGAA ACCAAUGU | 12022 |
| 2965 | GUCACCAUC UCAAUGUG | 1920 | CACAUUGA CUGAUGAGGCCGUUAGGCCGAA AUGGUGAC | 11339 |
| 2967 | CACCAUCUC AAUGUGGU | 1921 | ACCACAUU CUGAUGAGGCCGUUAGGCCGAA AGAUGGUG | 11340 |
| 2982 | GUGAACCUC CUAGGCGC | 2607 | GCGCCUAG CUGAUGAGGCCGUUAGGCCGAA AGGUUCAC | 12023 |
| 2985 | AACCUCCUA GGCGCCUG | 2608 | CAGGCGCC CUGAUGAGGCCGUUAGGCCGAA AGGAGGUU | 12024 |
| 3013 | GAGGGCCUC UCAUGGUG | 2609 | CACCAUGA CUGAUGAGGCCGUUAGGCCGAA AGGCCCUC | 12025 |
| 3015 | GGGCCUCUC AUGGUGAG | 2610 | AUCACCAU CUGAUGAGGCCGUUAGGCCGAA AGAGGCCC | 12026 |
| 3024 | AUGGUGAUG GUGGAAUU | 1928 | AAUUCCAC CUGAUGAGGCCGUUAGGCCGAA AUCACCAU | 11347 |
| 3032 | UGUGGAAUU CUGCAAGU | 2611 | ACUUGCAG CUGAUGAGGCCGUUAGGCCGAA AUUCCACA | 12027 |
| 3033 | GUGGAAUUC UGCAAGUU | 2612 | AACUUGCA CUGAUGAGGCCGUUAGGCCGAA AAUUCCAC | 12028 |
| 3041 | CUGCAAGUU UGGAAACC | 2613 | GGUUUCCA CUGAUGAGGCCGUUAGGCCGAA ACUGUCAG | 12029 |
| 3042 | UGCAAGUUU GGAAACCU | 2614 | AGGUUUCC CUGAUGAGGCCGUUAGGCCGAA AACUUGCA | 12030 |
| 3051 | GGAAACCUA UCAACUUA | 2615 | UAAGUUGA CUGAUGAGGCCGUUAGGCCGAA AGGUUUCC | 12031 |
| 3053 | AAACCUAUC AACUUACU | 2616 | AGUAAGUU CUGAUGAGGCCGUUAGGCCGAA AUAGGUUU | 12032 |
| 3058 | UAUCAACUU ACUUACGG | 2617 | CCGUAAGU CUGAUGAGGCCGUUAGGCCGAA AGUUGAUA | 12033 |
| 3059 | AUCAACUUA CUUACGGG | 2618 | CCCGUAAG CUGAUGAGGCCGUUAGGCCGAA AAGUUGAU | 12034 |
| 3062 | AACUUACUU ACGGGGCA | 2619 | UGCCCCGU CUGAUGAGGCCGUUAGGCCGAA AGUAAGUU | 12035 |
| 3063 | ACUUACUUA CGGGGCAA | 2620 | UUGCCCCG CUGAUGAGGCCGUUAGGCCGAA AAGUAAGU | 12036 |
| 3083 | AAAUGAAUU UGUUCCCU | 2621 | AGGGAACA CUGAUGAGGCCGUUAGGCCGAA AUUCAUUU | 12037 |
| 3084 | AAUGAAUUU GUUCCCUA | 2622 | UAGGGAAC CUGAUGAGGCCGUUAGGCCGAA AAUUCAUU | 12038 |
| 3087 | GAAUUUGUU CCCUAUAA | 2623 | UUAUAGGG CUGAUGAGGCCGUUAGGCCGAA ACAAAUUC | 12039 |
| 3088 | AAUUUGUUC CCUAUAAG | 2624 | CUUAUAGG CUGAUGAGGCCGUUAGGCCGAA AACAAAUU | 12040 |
| 3092 | UGUUCCCUA UAAGAGCA | 2625 | UGCUCUUA CUGAUGAGGCCGUUAGGCCGAA AGGGAACA | 12041 |
| 3094 | UUCCCUAUA AGAGCAAA | 2626 | UUUGCUCU CUGAUGAGGCCGUUAGGCCGAA AUAGGGAA | 12042 |
| 3113 | GGCACGCUU CCGCCAGG | 2627 | CCUGGCGG CUGAUGAGGCCGUUAGGCCGAA AGCGUGCC | 12043 |
| 3114 | GCACGCUUC CGCCAGGG | 2628 | CCCUGGCG CUGAUGAGGCCGUUAGGCCGAA AAGCGUGC | 12044 |
| 3131 | CAAGGACUA CGUUGGGG | 2629 | CCCCAACG CUGAUGAGGCCGUUAGGCCGAA AGUCCUUG | 12045 |
| 3135 | GACUACGUU GGGGAGCU | 2630 | AGCUCCCC CUGAUGAGGCCGUUAGGCCGAA ACGUAGUC | 12046 |
| 3144 | GGGGAGCUC UCCGUGGA | 2631 | UCCACGGA CUGAUGAGGCCGUUAGGCCGAA AGCUCCCC | 12047 |
| 3146 | GGAGCUCUC CGUGGAUC | 2632 | GAUCCACG CUGAUGAGGCCGUUAGGCCGAA AGAGCUCC | 12048 |
| 3154 | CCGUGGAUC UGAAAAGA | 2633 | UCUUUUCA CUGAUGAGGCCGUUAGGCCGAA AUCCACGG | 12049 |
| 3167 | AAGACGCUU GGACAGCA | 2634 | UGCUGUCC CUGAUGAGGCCGUUAGGCCGAA AGCGUCUG | 12050 |
| 3177 | GACAGCAUC ACCAGCAG | 2635 | CUGCUGGU CUGAUGAGGCCGUUAGGCCGAA AUGCUGUC | 12051 |
| 3194 | CCAGAGCUC UGCCAGCU | 2636 | AGCUGGCA CUGAUGAGGCCGUUAGGCCGAA AGCUCUGG | 12052 |
| 3203 | UGCCAGCUC AGGCUUUG | 2637 | CAAAGCCU CUGAUGAGGCCGUUAGGCCGAA AGCUGGCA | 12053 |
| 3209 | CUCAGGCUU UGUUGAGG | 2638 | CCUCAACA CUGAUGAGGCCGUUAGGCCGAA AGCCUGAG | 12054 |
| 3210 | UCAGGCUUU GUUGAGGA | 2639 | UCCUCAAC CUGAUGAGGCCGUUAGGCCGAA AAGCCUGA | 12055 |
| 3213 | GGCUUUGUU GAGGAGAA | 2640 | UUCUCCUC CUGAUGAGGCCGUUAGGCCGAA ACAAAGCC | 12056 |
| 3224 | GGAGAAAUC GCUCAGUG | 2641 | CACUGAGC CUGAUGAGGCCGUUAGGCCGAA AUUUCUCC | 12057 |
| 3228 | AAAUCGCUC AGUGAUGU | 2642 | ACAUCACU CUGAUGAGGCCGUUAGGCCGAA AGCGAUUU | 12058 |
| 3237 | AGUGAUGUA GAGGAAGA | 2643 | UCUUCCUC CUGAUGAGGCCGUUAGGCCGAA ACAUCACU | 9921 |
| 3253 | AAGAAGCUU CUGAAGAA | 2644 | UUCUUCAG CUGAUGAGGCCGUUAGGCCGAA AGCUUCUU | 12059 |
| 3254 | AGAAGCUUC UGAAGAAC | 2645 | GUUCUUCA CUGAUGAGGCCGUUAGGCCGAA AAGCUUCU | 12060 |
| 3266 | AGAACUGUA CAAGGACU | 2646 | AGUCCUUG CUGAUGAGGCCGUUAGGCCGAA ACAGUUCU | 12061 |
| 3275 | CAAGGACUU CCUGACCU | 2647 | AGGUCAGG CUGAUGAGGCCGUUAGGCCGAA AGUCCUUG | 12062 |
| 3276 | AAGGACUUC CUGACCUG | 1962 | AAGGUCAG CUGAUGAGGCCGUUAGGCCGAA AAGUCCUU | 11381 |
| 3284 | CCUGACCUU GGAGCAUC | 1963 | GAUGCUCC CUGAUGAGGCCGUUAGGCCGAA AGGUCAGG | 11382 |
| 3292 | UGGAGCAUC UCAUCUGU | 1964 | ACAGAUGA CUGAUGAGGCCGUUAGGCCGAA AUGCUCCA | 11383 |
| 3294 | GAGCAUCUC AUCUGUUA | 1965 | UAACAGAU CUGAUGAGGCCGUUAGGCCGAA AGAUGCUC | 11384 |
| 3297 | CAUCUCAUC UGUGACAG | 1966 | CUGUAACA CUGAUGAGGCCGUUAGGCCGAA AUGAGAUG | 11385 |
| 3301 | UCAUCUGUG ACAGCUUC | 1967 | GAAGCUGU CUGAUGAGGCCGUUAGGCCGAA ACAGAUGA | 11386 |
| 3302 | CAUCUGUGA CAGCUUCC | 1968 | GGAAGCUG CUGAUGAGGCCGUUAGGCCGAA AACAGAUG | 11387 |
| 3308 | UUACAGCUU CCAAGUGG | 1969 | CCACUUGG CUGAUGAGGCCGUUAGGCCGAA AGCUGUAA | 11388 |
| 3309 | UACAGCUUC CAAGUGGC | 1970 | GCCACUUG CUGAUGAGGCCGUGAGGCCGAA AAGCUGUA | 11389 |
| 3319 | AAGUGGCUA AGGGCAUG | 1971 | CAUGCCCU CUGAUGAGGCCGUUAGGCCGAA AGCCACUU | 11390 |
| 3332 | CAUGGAGUU CUUGGCAU | 1972 | AUGCCAAG CUGAUGAGGCCGUUAGGCCGAA ACUCCAUG | 11391 |
| 3333 | AUGGAGUUC UUGGCAUC | 1973 | GAUGCCAA CUGAUGACGCCGUUAGGCCGAA AACUCCAU | 11392 |
| 3335 | GGAGUUCUU GGCAUCAA | 2648 | UUGAUCCC CUCAUGAGGCCGUUAGGCCCAA AGAACUCC | 12063 |
| 3341 | CUUGGCAUC AAGGAAGU | 2649 | ACUUCCUU CUGAUGAGGCCGUUAGGCCGAA AUGCCAAG | 12064 |
| 3352 | CCAAGUGUA UCCACAGG | 2650 | CCUGUGGA CUGAUGAGGCCGUUAGGCCGAA ACACUUCC | 12065 |
| 3354 | AAGUGUAUC CACAGGGA | 1977 | UCCCUGUG CUGAUGAGGCCGUUAGGCCGAA AUACACUU | 11396 |
| 3381 | CGAAACAUU CUCCUAUC | 2651 | GAUAGGAG CUGAUGAGGCCGUUAGGCCGAA AUGUUUCG | 12066 |
| 3382 | GAAACAUUC UCCUAUCG | 2652 | CGAUAGGA CUGAUGAGGCCGUUAGGCCGAA AAUGUUUC | 12067 |
| 3384 | AACAUUCUC CUAUCGGA | 2653 | UCCGAUAG CUGAUGAGGCCGUUAGCCCGAA AGAAUGUU | 12068 |
| 3387 | AUUCUCCUA UCUGAGAA | 2654 | UUCUCCGA CUGAUGAGGCCGUUAGGCCGAA AGGAGAAU | 12069 |
| 3389 | UCUCCUAUC GGAGAAGA | 2655 | UCUUCUCC CUGAUGAGGCCGUUAGCCCGAA AHAGGAGA | 12070 |
| 3405 | AAUGUGGUU AAGAUCUG | 2656 | CAGAUCUU CUGAUGAGGCCGUUAGGCCGAA ACCACAUU | 12071 |
| 3406 | AUGUGGUUA AGAUCUGU | 2657 | ACAGAUCU CUGAUGAGGCCGUUAGGCCGAA AACCACAU | 12072 |
| 3411 | GUUAAGAUC UGUGACUU | 2658 | AAGUCACA CUGAUGAGGCCGUUAGGCCGAA AUCUUAAC | 12073 |
| 3419 | CUGUGACUU CGGCUUGG | 2659 | CCAAGCCG CUGAUGAGGCCGUUAGGCCGAA AGUCACAG | 12074 |
| 3420 | UGUGACUUC GGCUUGGC | 2660 | GCCAAGCC CUGAUGAGGCCGUUAGGCCGAA AAGUCACA | 11407 |
| 3425 | CUUCGGCUU GGCCCGGG | 2661 | CCCGGGCC CUGAUGAGGCCGUUAGGCCGAA AGCCGAAG | 12075 |
| 3438 | CGGGACAUU UAUAAAGA | 2662 | UCUUUAUA CUGAUGAGGCCGUUAGGCCGAA AUGUCCCG | 12076 |
| 3439 | GGGACAUUU AUAAAGAC | 2663 | GUCUUUAU CUGAUGAGGCCGUUAGGCCGAA AAUGUCCC | 12077 |
| 3440 | GGACAUUUA UAAAGACC | 2664 | GGUCUUUA CUGAUGAGGCCGUUAGGCCGAA AAAUGUCC | 12078 |
| 3442 | ACAUUUAUA AAGACCCG | 2665 | CGGGUCUU CUGAUGAGGCCGUUAGGCCGAA AUAAAUGU | 12079 |
| 3454 | ACCCGGAUU AUGUCAGA | 2666 | UCUGACAU CUGAUGAGGCCGUUAGGCCGAA AUCCGGGU | 12080 |
| 3455 | CCCGGAUUA UGUCAGAA | 2667 | UUCUGACA CUGAUGAGGCCGUUAGGCCGAA AAUCCGGG | 12081 |
| 3459 | GAUUAUGUC AGAAAAGG | 1998 | CCUUUUCU CUGAUGAGGCCGUUAGGCCGAA ACAUAAUC | 11417 |
| 3480 | GCCCGACUC CCUUUGAA | 2668 | UUCAAAGG CUGAUGAGGCCGUUAGGCCGAA AGUCGGGC | 12082 |
| 3484 | GACUCCCUU UGAAGUGG | 2669 | CCACUUCA CUGAUGAGGCCGUUAGGCCGAA AGGGAGUC | 12083 |
| 3485 | ACUCCCUUU GAAGUGGA | 2670 | UCCACUUC CUGAUGAGGCCGUUAGGCCGAA AAGGGAGU | 12084 |
| 3510 | GAAACCAUU UUUGACAG | 2671 | CUGUCAAA CUGAUGAGGCCGUUAGGCCGAA AUGGUUUC | 12085 |
| 3511 | AAACCAUUU UUGACAGA | 2672 | UCUGUCAA CUGAUGAGGCCGUUAGGCCGAA AAUGGUUU | 12086 |
| 3512 | AACCAUUUU UGACAGAG | 2673 | CUCUGUCA CUGAUGAGGCCGUUAGGCCGAA AAAUGGUU | 12087 |
| 3513 | ACCAUUUUU GACAGAGU | 2674 | ACUCUGUC CUGAUGAGGCCGUUAGGCCGAA AAAAUGGU | 12088 |
| 3522 | GACAGAGUA UACACAAU | 2675 | AUUGUGUA CUGAUGAGGCCGUUAGGCCGAA ACUCUGUC | 12089 |
| 3524 | CAGAGUAUA CACAAUUC | 2676 | GAAUUGUG CUGAUGAGGCCGUUAGGCCGAA AUACUCUG | 12090 |
| 3531 | UACACAAUU CAGAGCGA | 2677 | UCGCUCUG CUGAUGAGGCCGUUAGGCCGAA AUUGUGUA | 12091 |
| 3532 | ACACAAUUC AGAGCGAU | 2678 | AUCGCUCU CUGAUGAGGCCGUUAGGCCGAA AAUUGUGU | 12092 |
| 3548 | UGUGUGGUC UUUCGGUG | 2679 | CACCGAAA CUGAUGAGGCCGUUAGGCCGAA ACCACACA | 12093 |
| 3550 | UGUGGUCUU UCUGUGUG | 2680 | CACACCGA CUGAUGAGGCCGUUAGGCCGAA AGACCACA | 12094 |
| 3551 | GUGGUCUUU CGGUGUGU | 2681 | ACACACCG CUGAUGAGGCCGUUAGGCCGAA AAGACCAC | 12095 |
| 3552 | UGGUCUUUC GGUGUGUU | 2682 | AACACACC CUGAUGAGGCCGUUAGGCCGAA AAAGACCA | 12096 |
| 3560 | CGGUGUGUU GCUCUGGG | 2683 | CCCAGAGC CUGAUGAGGCCGUUAGGCCGAA ACACACCG | 12097 |
| 3564 | GUGUUGCUC UGGGAAAU | 2684 | AUUUCCCA CUGAUGAGGCCGUUAGGCCGAA AGCAACAC | 12098 |
| 3573 | UGGGAAAUA UUUUCCUU | 2017 | AAGGAAAA CUGAUGAGGCCGUUAGGCCGAA AUUUCCCA | 11436 |
| 3575 | GGAAAUAUU UUCCUUAG | 2018 | CUAAGGAA CUGAUGAGGCCGUUAGGCCGAA AUAUUUCC | 11437 |
| 3576 | GAAAUAUUU UCCUUAGG | 2019 | CCUAAGGA CUGAUGAGGCCGUUAGGCCGAA AAUAUUUC | 11438 |
| 3577 | AAAUAUUUU CCUUAGGU | 2020 | ACCUUAGG CUGAUGAGGCCGUUAGGCCGAA AAAUAUUU | 11439 |
| 3578 | AAUAUUUUC CUUAGGUG | 2021 | CACCUAAG CUGAUGAGGCCGUUAGGCCGAA AAAAUAUU | 11440 |
| 3581 | AUUUUCCUU AGGUGCCU | 2685 | AGGCACCU CUGAUGAGGCCGUUAGGCCGAA AGGAAAAU | 12099 |
| 3582 | UUUUCCUUA GGUGCCUC | 2686 | GAGGCACC CUGAUGAGGCCGUUAGGCCGAA AAGGAAAA | 12100 |
| 3590 | AGGUGCCUC CCCAUACC | 2687 | GGUAUGGG CUGAUGAGGCCGUUAGGCCGAA AGGCACCU | 12101 |
| 3596 | CUCCCCAUA CCCUGGGG | 2688 | CCCCAGGG CUGAUGAGGCCGUUAGGCCGAA AUGUGGAG | 12102 |
| 3606 | CCUGGGGUC AAGAUUGA | 2689 | UCAAUCUU CUGAUGAGGCCGUUAGGCCGAA ACCCCAGG | 11448 |
| 3612 | GUCAAGAUU GAUGAAGA | 2690 | UCUUCAUC CUGAUGAGGCCGUUAGGCCGAA AUCUUGAC | 12103 |
| 3623 | UGAAGAAUU UUGUAGGA | 2691 | UCCUACAA CUGAUGAGGCCGUUAGGCCGAA AUUCUUCA | 12104 |
| 3624 | GAACAAUUU UGUAGGAG | 2692 | CUCCUACA CUGAUGAGGCCGUUAGGCCGAA AAUUCUUC | 12105 |
| 3625 | AAGAAUUUU GUAGGAGA | 2693 | UCUCCUAC CUGAUGAGGCCGUUAGGCCGAA AAAUUCUU | 12106 |
| 3628 | AAUUUUGUA GGAGAUUG | 2694 | CAAUCUCC CUGAUGAGGCCGUUAGGCCGAA ACAAAAUU | 12107 |
| 3635 | UAGGAGAUU GAAAGAAG | 2695 | CUUCUUUC CUGAUGAGGCCGUUAGGCCGAA AUCUCCUA | 12108 |
| 3649 | AAGGAACUA GAAUGCGG | 2696 | CCGCAUUC CUGAUGAGGCCGUUAGGCCGAA AGUUCCUU | 12109 |
| 3661 | UGCGGGCUC CUGACUAC | 2697 | GUAGUCAG CUGAUGAGGCCGUUAGGCCGAA AGCCCGCA | 12110 |
| 3668 | UCCUGACUA CACUACCC | 2698 | GGGUAGUG CUGAUGAGGCCGUUAGGCCGAA AGUCAGGA | 12111 |
| 3673 | ACUACACUA CCCCAGAA | 2699 | UUCUGGGG CUGAUGAGGCCGUUAGGCCGAA AGUGUAGU | 12112 |
| 3686 | AGAAAUGUA CCAGACCA | 2041 | UGGUCUGG CUGAUGAGGCCGUUAGGCCGAA ACAUUUCU | 11460 |
| 3734 | GAGACCCUC GUUUUCAG | 2700 | CUGAAAAC CUGAUGAGGCCGUUAGGCCGAA AGGGUCUC | 12113 |
| 3737 | ACCCUCGUU UUCAGAGU | 2701 | ACUCUGAA CUGAUGAGGCCGUUAGGCCGAA ACGAGGGU | 12114 |
| 3738 | CCCUCGUUU UCAGAGUU | 2702 | AACUCUGA CUGAUGAGGCCGUUAGGCCGAA AACGAGGG | 12115 |
| 3739 | CCUCGUUUU CAGAGUUG | 2703 | CAACUCUG CUGAUGAGGCCGUUAGGCCGAA AAACGAGG | 12116 |
| 3740 | CUCGUUUUC AGAGUUGG | 2704 | CCAACUCU CUGAUGAGGCCGUUAGGCCGAA AAAACGAG | 12117 |
| 3746 | UUCAGAGUU GGUGGAGC | 2705 | GCUCCACC CUGAUGAGGCCGUUAGGCCGAA ACUCUGAA | 12118 |
| 3757 | UGGAGCAUU UGGGAAAC | 2706 | GUUUCCCA CUGAUGAGGCCGUUAGGCCGAA AUGCUCCA | 12119 |
| 3758 | GGAGCAUUU GGGAAACC | 2707 | GGUUUCCC CUGAUGAGGCCGUUAGGCCGAA AAUGCUCC | 12120 |
| 3768 | GGAAACCUC CUGCAAGC | 2708 | GCUUGCAG CUGAUGAGGCCGUUAGGCCGAA AGGUUUCC | 12121 |
| 3803 | CAAAGACUA UAUUGUUC | 2709 | GAACAAUA CUGAUGAGGCCGUUAGGCCGAA AGUCUUUG | 12122 |
| 3805 | AAGACUAUA UUGUUCUU | 2710 | AAGAACAA CUGAUGAGGCCGUUAGGCCGAA AUAGUCUU | 12123 |
| 3807 | GACUAUAUU GUUCUUCC | 2711 | GGAAGAAC CUGAUGAGGCCGUUAGGCCGAA AUAUAGUC | 12124 |
| 3810 | UAUAUUGUU CUUCCAAU | 2712 | AUUGGAAG CUGAUGAGGCCGUUAGGCCGAA ACAAUAUA | 12125 |
| 3811 | AUAUUGUUC UUCCAAUG | 2713 | CAUUGGAA CUGAUGAGGCCGUUAGGCCGAA AACAAUAU | 12126 |
| 3813 | AUUGUUCUU CCAAUGUC | 2714 | GACAUUGG CUGAUGAGGCCGUUAGGCCGAA AGAACAAU | 12127 |
| 3814 | UUGUUCUUC CAAUGUCA | 2715 | UGACAUUG CUGAUGAGGCCGUUAGGCCGAA AAGAACAA | 12128 |
| 3821 | UCCAAUGUC AGAGACAC | 2716 | GUGUCUCU CUGAUGAGGCCGUUAGGCCGAA ACAUUGGA | 12129 |
| 3847 | AAGAGGAUU CUGGACUC | 2065 | GAGUCCAG CUGAUGAGGCCGUUAGGCCGAA AUCCUCUU | 11484 |
| 3848 | AGAGGAUUC UGGACUCU | 2066 | AGAGUCCA CUGAUGAGGCCGUUAGGCCGAA AAUCCUCU | 11485 |
| 3855 | UCUGGACUC UCCCUGCC | 2717 | GGCAGGGA CUGAUGAGGCCGUUAGGCCGAA AGUCCAGA | 12130 |
| 3857 | UGGACUCUC CCUGCCUA | 2718 | UAGGCAGG CUGAUGAGGCCGUUAGGCCGAA AGAGUCCA | 12131 |
| 3865 | CCCUGCCUA CCUCACCU | 2719 | AGGUGAGG CUGAUGAGGCCGUUAGGCCGAA AGGCAGGG | 12132 |
| 3869 | GCCUACCUC ACCUGUUU | 2071 | AAACAGGU CUGAUGAGGCCGUUAGGCCGAA AGGUAGGC | 11490 |
| 3876 | UCACCUGUU UCCUGUAU | 2072 | AUACAGGA CUGAUGAGGCCGUUAGGCCGAA ACAGGUGA | 11491 |
| 3877 | CACCUGUUU CCUGUAUG | 2073 | CAUACAGG CUGAUGAGGCCGUUAGGCCGAA AACAGGUG | 11492 |
| 3878 | ACCUGUUUC CUGUAUGG | 2074 | CCAUACAG CUGAUGAGGCCGUUAGGCCGAA AAACAGGU | 11493 |
| 3883 | UUUCCUGUA UGGAGGAA | 2720 | UUCCUCCA CUGAUGAGGCCGUUAGGCCGAA ACAGGAAA | 12133 |
| 3914 | CCCCAAAUU CCAUUAUG | 2077 | CAUAAUGG CUGAUGAGGCCGUUAGGCCGAA AUUUGGGG | 11496 |
| 3915 | CCCAAAUUC CAUUAUGA | 2078 | UCAUAAUG CUGAUGAGGCCGUUAGGCCGAA AAUUUGGG | 11497 |
| 3919 | AAUUCCAUU AUGACAAC | 2079 | GUUGUCAU CUGAUGAGGCCGUUAGGCCGAA AUGGAAUU | 11498 |
| 3920 | AUUCCAUUA UGACAACA | 2080 | UGUUGUCA CUGAUGAGGCCGUUAGGCCGAA AAUGGAAU | 11499 |
| 3939 | GCAGGAAUC AGUCAUUA | 2721 | UAAUGACU CUGAUGAGGCCGUUAGGCCGAA AUUCCUGC | 12134 |
| 3943 | GAAUCAGUC AUUAUCUC | 2722 | GAGAUAAU CUGAUGAGGCCGUUAGGCCGAA ACUGAUUC | 12135 |
| 3946 | UCAGUCAUU AUCUCCAG | 2723 | CUGGAGAU CUGAUGAGGCCGUUAGGCCGAA AUGACUGA | 12136 |
| 3947 | CAGUCAUUA UCUCCAGA | 2724 | UCUGGAGA CUGAUGAGGCCGUUAGGCCGAA AAUGACUG | 12137 |
| 3949 | GUCAUHAUC UCCAGAAC | 2725 | GUUCUGGA CUGAUGAGGCCGUUAGGCCGAA AUAAUGAC | 12138 |
| 3951 | CAUHAUCUC CAGAACAG | 2726 | CUGUUCUG CUGAUGAGGCCGUUAGGCCGAA AGAUAAUG | 12139 |
| 3961 | AGAACAGUA AGCGAAAG | 2085 | CUUUCGCU CUGAUGAGGCCGUUAGGCCGAA ACUGUUCU | 11504 |
| 3987 | GUGAGUGUA AAAACAUU | 2086 | AAUGUUUU CUGAUGAGGCCGUUAGGCCGAA ACACUCAC | 11505 |
| 3995 | AAAAACAUU UGAAGAUA | 2087 | UAUCUUCA CUGAUGAGGCCGUUAGGCCGAA AUGUUUUU | 11506 |
| 3996 | AAAACAUUU GAAGAUAU | 2088 | AUAUCUUC CUGAUGAGGCCGUUAGGCCGAA AAUGUUUU | 11507 |
| 4003 | UUGAAGAUA UCCCAUUG | 2727 | CAAUGGGA CUGAUGAGGCCGUUAGGCCGAA AUCUUCAA | 12140 |
| 4005 | GAAGAUAUC CCAUUGGA | 2728 | UCCAAUGG CUGAUGAGGCCGUUAGGCCGAA AUAUCUUC | 12141 |
| 4010 | UAUCCCAUU GGAGCAAC | 2729 | GUUCCUCC CUGAUGAGGCCGUUAGGCCGAA AUUGGAUA | 12142 |
| 4026 | CCAGAAGUA AAAGUGAU | 2730 | AUCACUUU CUGAUGAGGCCGUUAGGCCGAA ACUUCUGC | 12143 |
| 4035 | AAAGUCAUC CCAGAUGA | 2731 | UCAUCUGG CUGAUGAGGCCGUUAGGCCGAA AUCACUUU | 12144 |
| 4068 | GGGAUGGUC CUUGCAUC | 2732 | GAUGCAAG CUGAUGAGGCCGUUAGGCCGAA ACCAUCCC | 12145 |
| 4071 | AUGGUCCUU GCAUCAGA | 2733 | UCUCAUGC CUGAUGAGGCCGUUAGGCCGAA AGGACCAU | 12146 |
| 4076 | CCUUCCAUC AGAAGAGC | 2734 | GCUCUUCU CUGAUGAGGCCGUUACGCCGAA AUGCAAGG | 12147 |
| 4093 | UGAAAACUC UGGAAGAC | 2735 | GUCUUCCA CUGAUGACGCCGUUAGGCCGAA AGUUUUCA | 11520 |
| 4112 | GAACAAAUU AUCUCCAU | 2736 | AUGGAGAU CUGAUGAGGCCGUUAGGCCGAA AUUUGUUC | 12148 |
| 4113 | AACAAAUUA UCUCCAUC | 2737 | GAUGGAGA CUGAUGAGGCCGUUAGGCCGAA AAUUUGUU | 12149 |
| 4115 | CAAAUUAUC UCCAUCUU | 2105 | AAGAUGGA CUGAUGAGGCCGUUAGGCCGAA AUAAUUUG | 11524 |
| 4117 | AAUUAUCUC CAUCUUUU | 2106 | AAAAGAUG CUGAUGAGGCCGUUAGCCCGAA AGAUAAUU | 11525 |
| 4121 | AUCUCCAUC UUUUGGUG | 2107 | CACCAAAA CUGAUGAGGCCGUUAGGCCGAA AUGGAGAU | 11526 |
| 4123 | CUCCAUCUU UUGGUGGA | 2108 | UCCACCAA CUGAUGAGGCCGUUAGGCCGAA AGAUGGAG | 11527 |
| 4124 | UCCAUCUUU UGGUGGAA | 2109 | UUCCACCA CUGAUGAGGCCGUUAGGCCGAA AAGAUGGA | 11528 |
| 4125 | CCAUCUUUU GGUGGAAU | 2110 | AUUCCACC CUGAUGAGGCCGUUAGGCCGAA AAAGAUGG | 11529 |
| 4144 | UGCCCAGUA AAAGCAGG | 2738 | CCUGCUUU CUGAUGAGGCCGUUAGGCCGAA ACUGGGCA | 12150 |
| 4157 | CAGGGAGUC UGUGGCCU | 2739 | AGGCCACA CUGAUGAGGCCGUUAGGCCGAA ACUCCCUG | 12151 |
| 4166 | UGUGGCCUC GGAAGGCU | 2740 | AGCCUUCC CUGAUGAGGCCGUUAGGCCGAA AGGCCACA | 12152 |
| 4175 | GGAAGGCUC CAACCAGA | 2741 | UCUGGUUG CUGAUGAGGCCGUUAGGCCGAA AGCCUUCC | 12153 |
| 4193 | CAGUGGCUA CCAGUCUG | 2742 | CAGACUGG CUGAUGAGGCCGUUAGGCCGAA AGCCACUG | 12154 |
| 4199 | CUACCAGUC UGGGUAUC | 2743 | GAUACCCA CUGAUGAGGCCGUUAGGCCGAA ACUGGUAG | 12155 |
| 4205 | GUCUGGGUA UCACUCAG | 2744 | CUGAGUGA CUGAUGAGGCCGUUAGGCCGAA ACCCAGAC | 12156 |
| 4207 | CUGGGUAUC ACUCAGAU | 2745 | AUCUGAGU CUGAUGAGGCCGUUAGGCCGAA AUACCCAG | 12157 |
| 4211 | GUAUCACUC AGAUGACA | 2746 | UGUCAUCU CUGAUGAGGCCGUUAGGCCGAA AGUGAUAC | 12158 |
| 4235 | CACCGUGUA CUCCAGCG | 2747 | CGCUGGAG CUGAUGAGGCCGUUAGGCCGAA ACACGGUG | 12159 |
| 4238 | CGUGUACUC CAGCGACG | 2748 | CGUCGCUG CUGAUGAGGCCGUUAGGCCGAA AGUACACG | 12160 |
| 4257 | GCAGGACUU UUAAAGAU | 2749 | AUCUUUAA CUGAUGAGGCCGUUAGGCCGAA AGUCCUGC | 12161 |
| 4258 | CAGGACUUU UAAAGAUG | 2750 | CAUCUUUA CUGAUGAGGCCGUUAGGCCGAA AAGUCCUG | 12162 |
| 4259 | AGGACUUUU AAAGAUGG | 2751 | CCAUCUUU CUGAUGAGGCCGUUAGGCCGAA AAAGUCCU | 12163 |
| 4260 | GGACUUUUA AAGAUGGU | 2752 | ACCAUCUU CUGAUGAGGCCGUUAGGCCGAA AAAAGUCC | 12164 |
| 4281 | GCUGCAGUU CACGCUGA | 2753 | UCAGCGUG CUGAUGAGGCCGUUAGGCCGAA ACUGCAGC | 12165 |
| 4282 | CUGCAGUUC ACGCUGAC | 2754 | GUCAGCGU CUGAUGAGGCCGUUAGGCCGAA AACUGCAG | 12166 |
| 4292 | CGCUGACUC AGGGACCA | 2755 | UGGUCCCU CUGAUGAGGCCGUUAGGCCGAA AGUCAGCG | 12167 |
| 4311 | CUGCAGCUC ACCUCCUG | 2756 | CAGGAGGU CUGAUGAGGCCGUUAGGCCGAA AGCUGCAG | 12168 |
| 4316 | GCUCACCUC CUGUUUAA | 2757 | UUAAACAG CUGAUGAGGCCGUUAGGCCGAA AGGUGAGC | 12169 |
| 4321 | CCUCCUGUU UAAAUGGA | 2758 | UCCAUUUA CUGAUGAGGCCGUUAGGCCGAA ACAGGAGO | 12170 |
| 4322 | CUCCUGUUU AAAUGGAA | 2759 | UUCCAUUU CUGAUGAGGCCGUUAGGCCGAA AACAGGAG | 12171 |
| 4323 | UCCUGUUUA AAUGGAAG | 2760 | CUUCCAUU CUGAUGAGGCCGUUAGGCCGAA AAACAGGA | 12172 |
| 4336 | GAAGUGGUC CUGUCCCG | 2761 | CGGGACAG CUGAUGAGGCCGUUAGGCCGAA ACCACHUC | 12173 |
| 4341 | GGUCCUGUC CCGGCUCC | 2762 | GGAGCCGG CUGAUGAGGCCGUUAGGCCGAA ACAGGACC | 12174 |
| 4348 | UCCCGGCUC CGCCCCCA | 2763 | UGGGGGCG CUGAUGAGGCCGUUAGGCCGAA AGCCGGGA | 12175 |
| 4360 | CCCCAACUC CUGGAAAU | 2764 | AUUUCCAG CUGAUGAGGCCGUUAGGCCGAA AGUUGGGG | 12176 |
| 4369 | CUGGAAAUC ACGAGAGA | 2765 | UCUCUCGU CUGAUGAGGCCGUUAGGCCGAA AUUUCCAG | 12177 |
| 4387 | GUGCUGCUU AGAUUUUC | 2766 | GAAAAUCU CUGAUGAGGCCGUUAGGCCGAA AGCAGCAC | 12178 |
| 4388 | UGCUGCUUA GAUUUUCA | 2767 | UGAAAAUC CUGAUGAGGCCGUUAGGCCGAA AAGCAGCA | 12179 |
| 4392 | GCUUAGAUU UUCAAGUG | 2768 | CACUUGAA CUGAUGAGGCCGUUAGGCCGAA AUCUAAGC | 12180 |
| 4393 | CUUAGAUUU UCAAGUGU | 2769 | ACACUUGA CUGAUGAGGCCGUUAGGCCGAA AAUCUAAG | 12181 |
| 4394 | UUAGAUUUU CAAGUGUU | 2770 | AACACUUG CUGAUGAGGCCGUUAGGCCGAA AAAUCUAA | 12182 |
| 4395 | UAGAUUUUC AAGUGUUG | 2771 | CAACACUU CUGAUGAGGCCGUUAGGCCGAA AAAAUCUA | 12183 |
| 4402 | UCAAGUGUU GUUCUUUC | 2772 | GAAAGAAC CUGAUGAGGCCGUUAGGCCGAA ACACUUGA | 12184 |
| 4405 | AGUGUUGUU CUUUCCAC | 2146 | GUGGAAAG CUGAUGAGGCCGUUAGGCCGAA ACAACACU | 11565 |
| 4406 | GUGUUGUUC UUUCCACC | 2147 | GGUGGAAA CUGAUGAGGCCGUUAGGCCGAA UACAACAC | 11566 |
| 4408 | GUUGUUCUU UCCACCAC | 2773 | GUGGUCCA CUGAUGAGGCCGUUAGGCCGAA AGAACAAC | 12185 |
| 4409 | UUGUUCUUU CCACCACC | 2774 | GGUGGUGG CUGAUGAGGCCGUUAGGCCGAA AAGAACAA | 12186 |
| 4410 | UGUUCUUUC CACCACCC | 2775 | GGGUGGUG CUGAUGAGGCCGUUAGGCCGAA AAAGAACA | 12187 |
| 4425 | CCGGAAGUA GCCACAUU | 2776 | AAUGUGGC CUGAUGAGGCCGUUAGGCCGAA ACUUCCGG | 12188 |
| 4433 | AUCCACAUG UGAUUUUC | 2777 | GAAAAUCA CUGAUGAGGCCGUUAGGCCGAA AUGUGGCU | 12189 |
| 4434 | GCCACAUUU GAUUUUCA | 2778 | UGAAAAUC CUGAUGAGGCCGUUAGGCCGAA AAUGUGGC | 12190 |
| 4438 | CAUGUGAUG UUCAUUUU | 2779 | AAAAUGAA CUGAUGAGGCCGUUAGGCCGAA AUCAAAUG | 12191 |
| 4439 | AUUUGAUUU UCAUUUUU | 2780 | AAAAAUGA CUGAUGAGGCCGUUAGGCCGAA AAUCAAAU | 12192 |
| 4440 | UUUGAUUUU CAUUUUUG | 2781 | CAAAAAUG CUGAUGAGGCCGUUAGGCCGAA AAAUCAAA | 12193 |
| 4441 | HUGAGUGUC AUGGUGGG | 2782 | CCAAAAAU CUGAUGAGGCCGUUAGGCCGAA AAAAUCAA | 12194 |
| 4444 | AUUUUCAUU UUUGGAGG | 2783 | CCUCCAAA CUGAUGAGGCCGUUAGGCCGAA AUGAAAAU | 12195 |
| 4445 | UUUUCAUUU DUGGAGGA | 2784 | UCCUCCAA CUGAUGAGGCCGUUAGGCCGAA AAUGAAAA | 12196 |
| 4446 | UUUCAUUUU UGGAGGAG | 2785 | CUCCUCCA CUGAUGAGGCCGUUAGGCCGAA AAAUGAAA | 12197 |
| 4447 | UUCAUUUUU GGAGGAGG | 2786 | CCUCCUCC CUGAUGAGGCCGUUAGGCCGAA AAAAUGAA | 12198 |
| 4461 | AUGGACCUC AGACUGCA | 2787 | UGCAGUCU CUGAUGAGGCCGUUAGGCCGAA AGGUCCCU | 12199 |
| 4477 | AAGGAGCUU GUCCUCAG | 2788 | CUGAGGAC CUGAUGAGGCCGUUAGGCCGAA AGCUCCUU | 12200 |
| 4480 | GAGCUUGUC CUCAGGGC | 2789 | GCCCUGAG CUGAUGAGGCCGUUAGGCCGAA ACAAGCUC | 12201 |
| 4483 | CUUGUCCUC AGGGCAUU | 2790 | AAUGCCCU CUGAUGAGGCCGUUAGGCCGAA AGGACAAG | 12202 |
| 4491 | CAGGGCAUU UCCAGAGA | 2791 | UCUCUGGA CUGAUGAGGCCGUUAGGCCGAA AUGCCCUG | 12203 |
| 4492 | AGGGCAUUU CCAGAGAA | 2792 | UUCUCUOG CUGAUGAGGCCGUUAGGCCGAA AAUGCCCU | 12204 |
| 4493 | GGGCAUUUC CAGAGAAG | 2793 | CUUCUCUG CUGAUGAGGCCGUUAGGCCGAA AAAUGCCC | 12205 |
| 4525 | GAAUGUGUU GACUCUAC | 2794 | GUAGAGUC CUGAUGAGGCCGUUAGGCCGAA ACACAGUC | 12206 |
| 4530 | UGUUGACUC UACUCUCU | 2795 | AGAGAGGA CUGAUGAGGCCGUUAGGCCGAA AGUCAACA | 12207 |
| 4532 | UUGACUCUA CUCUCUUU | 2796 | AAAGAGAG CUGAUGAGGCCGUUAGGCCGAA AGAGUCAA | 12208 |
| 4535 | ACUCUACUC UCUUUUCC | 2797 | GGAAAAGA CUGAUGAGGCCGUUAGGCCGAA AGUAGAGU | 12209 |
| 4537 | UCUACUCUC UUUUCCAU | 2798 | AUGGAAAA CUGAUGAGGCCGUUAGGCCGAA AGAGUAGA | 12210 |
| 4539 | UACUCUCUU UUCCAUUC | 2799 | GAAUGGAA CUGAUGAGGCCGUUAGGCCGAA AGAGAGUA | 12211 |
| 4540 | ACUCUCUUU UCCAUUCA | 2800 | UGAAUGGA CUGAUGAGGCCGUUAGGCCGAA AAGAGAGU | 12212 |
| 4541 | CUCUCUUUG CCAUUCAU | 2801 | AUGAAUGG CUGAUGAGGCCGUUAGGCCGAA AAAGAGAG | 12213 |
| 4542 | UCUCUUUUC CAUUCAUU | 2802 | AAUGAAUG CUGAUGAGGCCGUUAGGCCGAA AAAAGAGA | 12214 |
| 4546 | UUUUCCAUU CAUUUAAA | 2803 | UUUAAAUG CUGAUGAGGCCGUUAGGCCGAA AUGGAAAA | 12215 |
| 4547 | UUUCCAUUC AUUUAAAA | 2804 | UUUUAAAU CUGAUGAGGCCGUUAGGCCGAA AAUGGAAA | 12216 |
| 4550 | CCAUUCAUU UAAAAGUC | 2805 | GACUUUUA CUGAUGAGGCCGUUAGGCCGAA AUGAAUGG | 12217 |
| 4551 | CAUUCAUUU AAAAGUCC | 2806 | GGACUUUU CUGAUGAGGCCGUUAGGCCGAA AAUGAAUG | 12218 |
| 4552 | AUUCAUUUA AAAGUCCU | 2807 | AGGACUUU CUGAUGAGGCCGUUAGGCCGAA AAAUGAAU | 12219 |
| 4558 | UUAAAAGUC CUAUAUAA | 2808 | UUAUAUAG CUGAUGAGGCCGUUAGGCCGAA ACUUUUAA | 12220 |
| 4561 | AAAGUCCUA UAUAAUGU | 2809 | ACAUUAUA CUGAUGAGGCCGUUAGGCCGAA AGGACUUU | 12221 |
| 4563 | AGUCCUAUA UAAUGUGC | 2810 | GCACAUUA CUGAUGAGGCCGUUAGGCCGAA AUAGGACU | 12222 |
| 4565 | UCCUAUAUA AUGUGCCC | 2811 | GGGCACAU CUGAUGAGGCCGUUAGGCCGAA AUAUAGGA | 12223 |
| 4583 | GCUGUGGUC UCACUACC | 2812 | GGUAGUGA CUGAUGAGGCCGUUAGGCCGAA ACCACAGC | 12224 |
| 4585 | UGUGGUCUC ACUACCAG | 2813 | CUGGUAGU CUGAUGAGGCCGUUAGGCCGAA AGACCACA | 12225 |
| 4589 | GUCUCACUA CCAGUUAA | 2814 | UUAACUGG CUGAUGAGGCCGUUAGGCCGAA AGUGAGAC | 12226 |
| 4595 | CUACCAGUG AAAGCAAA | 2815 | UUUGCUUU CUGAUGAGGCCGUUAGGCCGAA ACUGGUAG | 12227 |
| 4596 | UACCAGUUA AAGCAAAA | 2816 | UUUUGCUU CUGAUGAGGCCGUUAGGCCGAA AACUGGUA | 12228 |
| 4609 | AAAAGACUU UCAAACAC | 2817 | GUGUUUGA CUGAUGAGGCCGUUAGGCCGAA AGUCUUUU | 12229 |
| 4610 | AAAGACUUU CAAACACG | 2818 | CGUGUUUG CUGAUGAGGCCGUUAGGCCGAA AAGUCUUU | 12230 |
| 4611 | AAGACUUUC AAACACGU | 2819 | ACGUGUUU CUGAUGAGGCCGUUAGGCCGAA AAAGUCUU | 12231 |
| 4625 | CGUGGACUC UGUCCUCC | 2820 | GGAGGACA CUGAUGAGGCCGUUAGGCCGAA AGUCCACG | 12232 |
| 4629 | GACUCUGUC CUCCAAGA | 2821 | UCUUGGAG CUGAUGAGGCCGUUAGGCCGAA ACAGAGUC | 12233 |
| 4632 | UCUGUCCUC CAAGAAGU | 2822 | ACUUCUUG CUGAUGAGGCCGUUAGGCCGAA AGGACAGA | 12234 |
| 4654 | CGGCACCUC UGUGAAAC | 2823 | GUUUCACA CUGAUGAGGCCGUUAGGCCGAA AGGUGCCG | 12235 |
| 4668 | AACUGGAUC GAAUGGGC | 2824 | GCCCAUUC CUGAUGAGGCCGUUAGGCCGAA AUCCAGUU | 12236 |
| 4683 | GCAAUGCUU UGUGUGUU | 2825 | AACACACA CUGAUGACGCCGUUAGGCCGAA AGCAUUGC | 12237 |
| 4684 | CAAUGCUUU GUGUGUUG | 2826 | CAACACAC CUGAUGAGGCCGUUAGGCCGAA AAGCAUUG | 12238 |
| 4691 | UUGUGUGUU GAGGAUGG | 2827 | CCAUCCUC CUGAUGAGGCCGUUAGGCCGAA ACACACAA | 12239 |
| 4709 | UGAGAUGUC CCAGGGCC | 2828 | GGCCCUGG CUGAUGAGGCCGUUAGGCCGAA ACAUCUCA | 12240 |
| 4722 | GUCCUAGUC UGUCUACC | 2829 | GGUAGACA CUGAUGAGGCCGUUAGGCCGAA ACUCGGCC | 12241 |
| 4726 | GAGUCUGUC UACCUUGG | 2830 | CCAAGGUA CUGAUGAGGCCGUUAGGCCGAA ACAGACUC | 12242 |
| 4728 | GUCUGUCUA CCUUGGAG | 2831 | CUCCAAGG CUGAUGAGGCCGUUAGGCCGAA AGACAGAC | 12243 |
| 4732 | GUCUACCUG GGAGGCUU | 2832 | AAGCCUCC CUGAUGAGGCCGUUAGGCCGAA AGGUAGAC | 12244 |
| 4740 | UGGAGGCUU UGUGGAGG | 2833 | CCUCCACA CUGAUGAGGCCGUUAGGCCGAA AGCCUCCA | 12245 |
| 4741 | GGAGGCUUU GUGGAGGA | 2834 | UCCUCCAC CUGAUGAGGCCGUUAGGCCGAA AAGCCUCC | 12246 |
| 4758 | UGCGGGCUA UGAGCCAA | 2835 | UUGGCUCA CUGAUGAGGCCGUUAGGCCGAA AGCCCGCA | 12247 |
| 4771 | CCAAGUGUU AAGUGUGG | 2836 | CCACACUU CUGAUGAGGCCGUUAGGCCGAA ACACUUGG | 12248 |
| 4772 | CAAGUGUUA AGUGUGGG | 2837 | CCCACACU CUGAUGAGGCCGUUAGGCCGAA AACACUUG | 12249 |
| 4811 | GCGCAAGUC GCUCGGAG | 2838 | CUCCGAGC CUGAUGAGGCCGUUAGGCCGAA ACUUGCGC | 12250 |
| 4815 | AAGUCGCUC GGAGAGCG | 2839 | CGCUCUCC CUGAUGAGGCCGUUAGGCCGAA AGCGACUU | 12251 |
| 4826 | AGAGCGGUU GGAGCCUG | 2840 | CAGGCUCC CUGAUGAGGCCGUUAGGCCGAA ACCGCUCU | 12252 |
| 4844 | AGAUGCAUU GUGCUGGC | 2841 | GCCAGCAC CUGAUGAGGCCGUUAGGCCGAA AUGCAUCU | 12253 |
| 4854 | UGCUGGCUC UGGUGGAG | 2842 | CUCCACCA CUGAUGAGGCCGUUAGGCCGAA AGCCAGCA | 12254 |
| 4870 | GGUGGGCUU GUGGCCUG | 2843 | CAGGCCAC CUGAUGAGGCCGUUAGGCCGAA AGCCCACC | 12255 |
| 4880 | UGGCCUGUC AGGAAACG | 2844 | CGUUUCCU CUGAUGAGGCCGUUAGGCCGAA ACAGGCCA | 12256 |
| 4908 | GGCAGGGUU UGGUUUUG | 2845 | CAAAACCA CUGAUGAGGCCGUUAGGCCGAA ACCCUGCC | 12257 |
| 4909 | GCAGGGUUU GGUUUGGG | 2846 | CCAAAACC CUGAUGAGGCCGUUAGGCCGAA AACCCUGC | 12258 |
| 4913 | GGUUUGGUU UUGGAAGG | 2847 | CCUUCCAA CUGAUGAGGCCGUUAGGCCGAA ACCAAACC | 12259 |
| 4914 | GUUUGGUUU UGGAAGGU | 2848 | ACCUUCCA CUGAUGAGGCCGUUAGGCCGAA AACCAAAC | 12260 |
| 4915 | UUUGGUUUU GGAAGGUU | 2849 | AACCUUCC CUGAUGAGGCCGUUAGGCCGAA AAACCAAA | 12261 |
| 4923 | UGGAAGGUU UGCGUGCU | 2850 | AGCACGCA CUGAUGAGGCCGUUAGGCCGAA ACCUUCCA | 12262 |
| 4924 | GGAAGGUUU GCGUGCUC | 2851 | GAGCACGC CUGAUGAGGCCGUUAGGCCGAA AACCUUCC | 12263 |
| 4932 | UGCGUGCUC UUCACAGU | 2852 | ACUGUGAA CUGAUGAGGCCGUUAGGCCGAA AGCACGCA | 12264 |
| 4934 | CGUGCUCUU CACAGUCG | 2853 | CGACUGUG CUGAUGAGGCCGUUAGGCCGAA AGAGCACG | 12265 |
| 4935 | GUGCUCUUC ACAGUCGG | 2854 | CCGACUGU CUGAUGAGGCCGUUAGGCCGAA AAGAGCAC | 12266 |
| 4941 | UUCACAGUC GGGUUACA | 2855 | UGUAACCC CUGAUGAGGCCGUUAGGCCGAA ACUGUGAA | 12267 |
| 4946 | AGUCGGGUU ACAGGCGA | 2856 | UCGCCUGU CUGAUGAGGCCGUUAGGCCGAA ACCCGACU | 12268 |
| 4947 | GUCGGGUUA CAGGCGAG | 2857 | CUCGCCUG CUGAUGAGGCCGUUAGGCCGAA AACCCGAC | 12269 |
| 4957 | AGGCGAGUU CCCUGUGG | 2858 | CCACAGGG CUGAUGAGGCCGUUAGGCCGAA ACUCGCCU | 12270 |
| 4958 | GGCGAGUUC CCUGUGGC | 2859 | GCCACAGG CUGAUGAGGCCGUUAGGCCGAA AACUCGCC | 12271 |
| 4969 | UGUGGCGUU UCCUACUC | 2860 | GAGUAGGA CUGAUGAGGCCGUUAGGCCGAA ACOCCACA | 12272 |
| 4970 | GUGGCGUUU CCUACUCC | 2861 | GGAGUAGG CUGAUGAGGCCGUUAGGCCGAA AACGCCAC | 12273 |
| 4971 | UGGCGUUUC CUACUCCU | 2862 | AGGAGUAG CUGAUGAGGCCGUUAGGCCGAA AAACGCCA | 12274 |
| 4974 | CGUUUCCUA CUCCUAAU | 2863 | AUUAGGAG CUGAUGAGGCCGUUAGGCCGAA AGGAAACG | 12275 |
| 4977 | UUCCUACUC CUAAUGAG | 2864 | CUCAUUAG CUGAUGAGGCCGUUAGGCCGAA AGUAGGAA | 12276 |
| 4980 | CUACUCCUA AUGAGAGU | 2865 | ACUCUCAU CUGAUGAGGCCGUUAGGCCGAA AGGAGUAG | 12277 |
| 4989 | AUGAGAGUU CCUUCCGG | 2866 | CCGGAAGG CUGAUGAGGCCGUUAGGCCGAA ACUCUCAU | 12278 |
| 4990 | UGAGAGUUC CUUCCGGA | 2867 | UCCGGAAG CUGAUGAGGCCGUUAGGCCGAA AACUCUCA | 12279 |
| 4993 | GAGUUCCUU CCGGACUC | 2868 | GAGUCCGG CUGAUGAGGCCGUUAGGCCGAA AGGAACUC | 12280 |
| 4994 | AGUUCCUUC CGGACUCU | 2869 | AGAGUCCG CUGAUGAGGCCGUUAGGCCGAA AAGGAACU | 12281 |
| 5001 | UCCGGACUC UUACGUGU | 2870 | ACACGUAA CUGAUGAGGCCGUUAGGCCGAA AGUCCGGA | 12282 |
| 5003 | CGGACUCUU ACGUGUCU | 2871 | AGACACGU CUGAUGAGGCCGUUAGGCCGAA AGAGUCCG | 12283 |
| 5004 | GGACUCUUA CGUGUCUC | 2872 | GAGACACG CUGAUGAGGCCGUUAGGCCGAA AAGAGUCC | 12284 |
| 5010 | GUACQUGUC UCCUGGCC | 2873 | GGCCAGGA CUGAUGAGGCCGUUAGGCCGAA ACACGUAA | 12285 |
| 5012 | ACGUGUCUC CUGGCCUG | 2874 | CAGGCCAG CUGAUGAGGCCGUUAGGCCGAA AGACACGU | 12286 |
| 5046 | AUGCAGCUU GCUCCUUC | 2875 | GAAGGAGC CUGAUGAGGCCGUUAGGCCGAA AGCUGCAU | 12287 |
| 5050 | AGCUUGCUC CUUCCUCA | 2876 | UGAGGAAG CUGAUGAGGCCGUUAGGCCGAA AGCAAGCU | 12288 |
| 5053 | UUGCUCCUU CCUCAUCU | 2877 | AGAUGAGG CUGAUGAGGCCGUUAGGCCGAA AGGAGCAA | 12289 |
| 5054 | UGCUCCUUC CUCAUCUC | 2878 | GAGAUGAC CUGAUGAGGCCGUUAGGCCGAA AAGGAGCA | 12290 |
| 5057 | UCCUUCCUC AUCUCUCA | 2879 | UGAGAGAU CUGAUGAGGCCGUUAGGCCGAA AGGAAGGA | 12291 |
| 5060 | UUCCUCAUC UCUCAGUC | 2880 | UCCUGAGA CUGAUGAGGCCGUUAGGCCGAA AUGAGGAA | 12292 |
| 5062 | CCUCAUCUC UCAGOCUG | 2881 | CAUCCUGA CUGAUGAGGCCGUUAGGCCGAA AGAUGAGO | 12293 |
| 5064 | UCAUCUCUC AGOCUGUG | 2882 | CACAGCCU CUGAUGAGGCCGUUAGGCCGAA AGAGAUGA | 12294 |
| 5076 | CUGUGCCUU AAUUCAGA | 2883 | UCUGAAUU CUGAUGAGGCCGUUAGGCCGAA AGGCACAG | 12295 |
| 5077 | UGUGCCUUA AUUCAGAA | 2884 | UUCUGAAU CUGAUGAGGCCGUUAGGCCGAA AAGGCACA | 12296 |
| 5080 | GCCUUAAUU CAGAACAC | 2885 | GUGUUCUG CUGAUGAGGCCGUUAGGCCGAA AUUAAGGC | 12297 |
| 5081 | CCUUAAUUC AGAACACC | 2886 | GGUGUUCU CUGAUGAGGCCGUUAGGCCGAA AAUUAAGG | 12298 |
| 5105 | AGGAACGUC GGCAGAGG | 2887 | CCUCUGCC CUGAUGAGGCCGUUAGGCCGAA ACGUUCCU | 12299 |
| 5116 | CAGAGGCUC CUGACGGG | 2888 | CCCGUCAG CUGAUGAGGCCGUUAGGCCGAA AGCCUCUG | 12300 |
| 5135 | CGAAGAAUU GUGAGAAC | 2889 | GUUCUCAC CUGAUGAGGCCGUUAGGCCGAA AUUCUUCG | 12301 |
| 5156 | CAGAAACUC AGGGUUUC | 2890 | GAAACCCU CUGAUGAGGCCGUUAGGCCGAA AGUUUCUG | 12302 |
| 5162 | CUCAGGGUU UCUGCUGG | 2891 | CCAGCAGA CUGAUGAGGCCGUUAGGCCGAA ACCCUGAG | 12303 |
| 5163 | UCAGGGUUU CUGCUGGG | 2892 | CCCAGCAG CUGAUGAGGCCGUUAGGCCGAA AACCCUGA | 12304 |
| 5164 | CAGGGUUUC UGCUGGGU | 2893 | ACCCAGCA CUGAUGAGGCCGUUAGGCCGAA AAACCCUG | 12305 |
| 5203 | UGGCAGGUC UGAGGGUU | 2894 | AACCCUCA CUGAUGAGGCCGUUAGGCCGAA ACCUGCCA | 12306 |
| 5211 | CUGAGGGUU CUCUGUCA | 2895 | UGACAGAG CUGAUGAGGCCGUUAGGCCGAA ACCCUCAG | 12307 |
| 5212 | UGAGGGUUC UCUGUCAA | 2896 | UUGACAGA CUGAUGAGGCCGUUAGGCCGAA AACCCUCA | 12308 |
| 5214 | AGGGUUCUC UGUCAAGU | 2897 | ACUUGACA CUGAUGAGGCCGUUAGGCCGAA AGAACCCU | 12309 |
| 5218 | UUCUCUGUC AAGUGGCG | 2898 | CGCCACUU CUGAUGAGGCCGUUAGGCCGAA ACAGAGAA | 12310 |
| 5229 | GUGGCGGUA AAGGCUCA | 2899 | UGAGCCUU CUGAUGAGGCCGUUAGGCCGAA ACCGCCAC | 12311 |
| 5236 | UAAAGGCUC AGGCUGGU | 2900 | ACCAGCCU CUGAUGAGGCCGUUAGGCCGAA AGCCUUUA | 12312 |
| 5247 | GCUGGUGUU CUUCCUCU | 2901 | AGAGGAAG CUGAUGAGGCCGUUAGGCCGAA ACACCAGC | 12313 |
| 5248 | CUGGUGUUC UUCCUCUA | 2902 | UAGAGGUA CUGAUGAGGCCGUUAGGCCGAA AACACCAG | 12314 |
| 5250 | GGUGUUCUU CCUCUAUC | 2903 | GAUAGAGG CUGAUGAGGCCGUUAGGCCGAA AGAACACC | 12315 |
| 5251 | GUGUUCUUC CUCUAUCU | 2904 | AGAUAGAG CUGAUGAGGCCGUUAGGCCGAA AAGAACAC | 12316 |
| 5254 | UUCUUCCUC UAUCUCCA | 2905 | UGGAGAUA CUGAUGAGGCCGUUAGGCCGAA AGGAAGAA | 12317 |
| 5256 | CUUCCUCUA UCUCCACU | 2906 | AGUGGAGA CUGAUGAGGCCGUUAGGCCGAA AGAGGAAG | 12318 |
| 5258 | UCCUCUAUC UCCACUCC | 2907 | GGAGUGGA CUGAUGAGGCCGUUAGGCCGAA AUAGAGGA | 12319 |
| 5260 | CUCUAUCUC CACUCCUG | 2908 | CAGGAGUG CUGAUGAGGCCGUUAGGCCGAA AGAUAGAG | 12320 |
| 5265 | UCUCCACUC CUGUCAGG | 2909 | CCUGACAG CUGAUGAGGCCGUUAGGCCGAA AGUGGAGA | 12321 |
| 5270 | ACUCCUGUC AGGCCCCC | 2910 | GGGGGCCU CUGAUGAGGCCGUUAGGCCGAA ACAGGAGU | 12322 |
| 5283 | CCCCAAGUC CUCAGUAU | 2911 | AUACUGAG CUGAUGAGGCCGUUAGGCCGAA ACUUGGGG | 12323 |
| 5286 | CAAGUCCUC AGUAUUUU | 2912 | AAAAUACU CUGAUGAGGCCGUUAGGCCGAA AGGACUUG | 12324 |
| 5290 | UCCUCAGUA UUUUAGCU | 2913 | AGCUAAAA CUGAUGAGGCCGUUAGGCCGAA ACUGAGGA | 12325 |
| 5292 | CUCAGUAUU UUAGCUUU | 2914 | AAAGCUAA CUGAUGAGGCCGUUAGGCCGAA AUACUGAG | 12326 |
| 5293 | UCAGUAUUU UAGCUUUG | 2915 | CAAAGCUA CUGAUGAGGCCGUUAGGCCGAA AAUACUGA | 12327 |
| 5294 | CAGUAUUUU AGCUUUGU | 2916 | ACAAAGCU CUGAUGAGGCCGUUAGGCCGAA AAAUACUG | 12328 |
| 5295 | AGUAUUUUA GCUUUGUG | 2917 | CACAAAGC CUGAUGAGGCCGUUAGGCCGAA AAAAUACU | 12329 |
| 5299 | UUUUAGCUU UGUGGCUU | 2918 | AAGCCACA CUGAUGAGGCCGUUAGGCCGAA AGCUAAAA | 12330 |
| 5300 | UUUAGCUUU GUGGCUUC | 2919 | GAAGCCAC CUGAUGAGGCCGUUAGGCCGAA AAGCUAAA | 12331 |
| 5307 | UUGUGGCUU CCUGAUGG | 2920 | CCAUCAGG CUGAUGAGGCCGUUAGGCCGAA AGCCACAA | 12332 |
| 5308 | UGUGGCUUC CUGAUGGC | 2921 | GCCAUCAG CUGAUGAGGCCGUUAGGCCGAA AAGCCACA | 12333 |
| 5325 | AGAAAAAUC UUAAUUGG | 2922 | CCAAUUAA CUGAUGAGGCCGUUAGGCCGAA AUUUUUCU | 12334 |
| 5327 | AAAAAUCUU AAUUGGUU | 2923 | AACCAAUU CUGAUGAGGCCGUUAGGCCGAA AGAUUUUU | 12335 |
| 5328 | AAAAUCUUA AUUGGUUG | 2924 | CAACCAAU CUGAUGAGGCCGUUAGGCCGAA AAGAUUUU | 12336 |
| 5331 | AUCUUAAUU GGUUGGUU | 2925 | AACCAACC CUGAUGAGGCCGUUAGGCCGAA AUUAAGAU | 12337 |
| 5335 | UAAUUGGUU GGUUUGCU | 2926 | AGCAAACC CUGAUGAGGCCGUUAGGCCGAA ACCAAUUA | 12338 |
| 5339 | UGGUUGGUU UGCUCUCC | 2927 | GGAGAGCA CUGAUGAGGCCGUUAGGCCGAA ACCAACCA | 12339 |
| 5340 | GGUUGGUUU GCUCUCCA | 2928 | UGGAGAGC CUGAUGAGGCCGUUAGGCCGAA AACCAACC | 12340 |
| 5344 | GGUUUGCUC UCCAGAUA | 2929 | UAUCUGGA CUGAUGAGGCCGUUAGGCCGAA AGCAAACC | 12341 |
| 5346 | UUUGCUCUC CAGAUAAU | 2930 | AUUAUCUU CUGAUGAGGCCGUUAGCCCGAA AGAGCAAA | 12342 |
| 5352 | CUCCAGAUA AUCACUAG | 2931 | CUAGUGAU CUGAUGAGGCCGUUAGGCCGAA AUCUGGAG | 12343 |
| 5355 | CAGAUAAUC ACUAGCCA | 2932 | UGGCUAGU CUGAUGAGGCCGUUAGGCCGAA AUUAUCUG | 12344 |
| 5359 | UAAUCACUA GCCAGAUU | 2933 | AAUCUGGC CUGAUGAGGCCGUUAGGCCGAA AGUGAUUA | 12345 |
| 5367 | AGCCAGAUU UCGAAAUU | 2934 | AAUUUCGA CUGAUGAGGCCGUUAGGCCGAA AUCUGGCU | 12346 |
| 5368 | GCCAGAUUU CGAAAUUA | 2935 | UAAUUUCG CUGAUGAGGCCGUUAGGCCGAA AAUCUGGC | 12347 |
| 5369 | CCAGAUUUC GAAAUUAC | 2936 | UUAAUUUC CUGAUGAGGCCGUUAGGCCGAA AAAUCUGG | 12348 |
| 5375 | UUCGAAAUU ACUUUUUA | 2937 | UAAAAAGU CUGAUGAGGCCGUUAGGCCGAA AUUUCGAA | 12349 |
| 5376 | UCGAAAUUA CUUUUUAG | 2938 | CUAAAAAG CUGAUGAGGCCGUUAGGCCGAA AAUUUCGA | 12350 |
| 5379 | AAAUUACUU UUUAGCCG | 2939 | CGGCUAAA CUGAUGAGGCCGUUAGGCCGAA AGUAAUUU | 12351 |
| 5380 | AAUUACUUU UUAGCCGA | 2940 | UCGGCUAA CUGAUGAGGCCGUUAGGCCGAA AAGUAAUU | 12352 |
| 5381 | AUUACUUUU UAGCCGAG | 2941 | CUCGGCUA CUGAUGAGGCCGUUAGGCCGAA AAAGUAAU | 12353 |
| 5382 | UUACUUUUU AGCCGAGG | 2942 | CCUCGGCU CUGAUGAGGCCGUUAGGCCGAA AAAAGUAA | 12354 |
| 5383 | UACUUUUUA GCCGAGGU | 2943 | ACCUCGGC CUGAUGAGGCCGUUAGGCCGAA AAAAAGUA | 12355 |
| 5392 | GCCGAGGUU AUGAUAAC | 2944 | GUUAUCAU CUGAUGAGGCCGUUAGGCCGAA ACCUCGGC | 12356 |
| 5393 | CCGAGGUUA UGAUAACA | 2945 | UGUGAUCA CUGAUGAGGCCGUUAGGCCGAA AACCUCGG | 12357 |
| 5398 | GUUAUGAUA ACAUCUAC | 2946 | GUAGAUGU CUGAUGAGGCCGUUAGGCCGAA AUCAUAAC | 12358 |
| 5403 | GAUAACAUC UACUGUAU | 2947 | AUACAGUA CUGAUGAGGCCGUUAGGCCGAA AUGUUAUC | 12359 |
| 5405 | UAACAUCUA CUGUAUCC | 2948 | GGAUACAG CUGAUGAGGCCGUUAGGCCGAA AGAUGUUA | 12360 |
| 5410 | UCUACUGUA UCCUUUAG | 2949 | CUAAAGGA CUGAUGAGGCCGUUAGGCCGAA ACAGUAGA | 12361 |
| 5412 | UACUGUAUC CUUUAGAA | 2950 | UUCUAAAG CUGAUGAGGCCGUUAGGCCGAA AUACAGUA | 12362 |
| 5415 | UGUAUCCUU UAGAAUUU | 2951 | AAAUUCUA CUGAUGAGGCCGUUAGGCCGAA AGGAUACA | 12363 |
| 5416 | GUAUCCUUU AGAAUUUU | 2952 | AAAAUUCU CUGAUGAGGCCGUUAGGCCGAA AAGGAUAC | 12364 |
| 5417 | UAUCCUUUA GAAUUUUA | 2953 | UAAAAUUC CUGAUGAGGCCGUUAGGCCGAA AAAGGAUA | 12365 |
| 5422 | UUUAGAAUU UUAACCUA | 2954 | UAGGUUAA CUGAUGAGGCCGUUAGGCCGAA AUUCUAAA | 12366 |
| 5423 | UUAGAAUUU UAACCUAU | 2955 | AUAGGUUA CUGAUGAGGCCGUUAGGCCGAA AAUUCUAA | 12367 |
| 5424 | UAGAAUUUU AACCUAUA | 2956 | UAUAGGUU CUGAUGAGGCCGUUAGGCCGAA AAAUUCUA | 12368 |
| 5425 | AGAAUUUUA ACCUAUAA | 2957 | UUAUAGGU CUGAUGAGcsGCCGUUAGGCCGAA AAAAUUCU | 12369 |
| 5430 | UUUAACCUA UAAAACUA | 2958 | UAGUUUUA CUGAUGAGGCCGUUAGGCCGAA AGGUUAAA | 12370 |
| 5432 | UAACCUAUA AAACUAUG | 2959 | CAUAGUUU CUGAUGAGGCCGUUAGGCCGAA AUAGGUUA | 12371 |
| 5438 | AUAAAACUA UGUCUACU | 2960 | AGUAGACA CUGAUGAGGCCGUUAGGCCGAA AGUUUUAU | 12372 |
| 5442 | AACUAUGUC UACUGGUU | 2961 | AACCAGUA CUGAUGAGGCCGUUAGGCCGAA ACAUAGUU | 12373 |
| 5444 | CUAUGUCUA CUGGUUUC | 2962 | GAAACCAG CUGAUGAGGCCGUUAGGCCGAA AGACAUAG | 12374 |
| 5450 | CUACUGGUU UCUGCCUG | 2963 | CAGGCAGA CUGAUGAGGCCGUUAGGCCGAA ACCAGUAG | 12375 |
| 5451 | UACUGGUUU CUGCCUGU | 2964 | ACAGGCAG CUGAUGAGGCCGUUAGGCCGAA AACCAGUA | 12376 |
| 5452 | ACUGGUUUG UGCCUGUG | 2965 | CACAGGCA CUGAUGAGGCCGUUAGGCCGAA AAACCAGU | 12377 |

Where “X” represents stem II region of a HH ribozyme (Hertel et al., 1992 Nucleic Acids Res. 20 3252). The length of stem II may be ≧ 2 base-pairs.
Underlined region can be any X sequence or linker, as described herein.

TABLE VII
Mouse flk-1 VEGF Receptor—Hairpin Ribozyme and Substrate Sequences

| | | Seq ID | | |
|---|---|---|---|---|
| Pos | Substrate | No | HP Ribozyme Sequences | Seq ID No |
| 74 | GGGCCCA GAC UGUGUCCC | 2966 | GGGACACA AGAA GGGCCC ACCACAGAAACACACGUUGUGGUACAUUACCUGGUA | 12378 |
| 88 | UCCCGCA CCC GGGAUAAC | 2967 | GUUAUCCC ACAA GCGGGA ACCACAGAAACACACGUUGUGGUACAUUACCUGGUA | 12379 |
| 105 | CCUGGCU CAC CCGAUUCC | 2968 | GGAAUCGG AGAA GCCAGG ACCACAGAAACACACGUUGUGGUACAUUACCUCGUA | 12380 |
| 110 | CUGACCO GAU UCCGCGGA | 2969 | UCCGCGGA AGAA CGUCAG ACCAGAGAAACACACGUUGUGGUACAUUACCUGGUA | 12381 |
| 125 | GGACACC GCU GACAGCCG | 2970 | CGGCUGUC AGAA CUCUCC ACCAGAGAAACACACGUUGUGGUACAUUACCUGGUA | 12382 |
| 132 | GCUGACA CCC GCCCCUGG | 2971 | CCACCCGC AGAA CUCAGC ACCAGAGAAACACACGUUGUGGUACAUUACCUGGUA | 12383 |
| 138 | AGCCCCG GCU GGAGCCAG | 2972 | CUGGCUCC AGAA GCGGCU ACCAGAGAAACACACGUUCUGGUACAUUACCUGGUA | 12384 |
| 175 | CUCCCCG GUC UUGCGCUG | 2973 | CAGCGCAA AGAA GGGGAG ACCAGAGAAACACACGUUCUGGUACAUUACCUGGUA | 12385 |
| 199 | CCAUACC GCC UCUGUGAC | 2974 | GUCACAGA AGAA GUAUGG ACCAGAGAAACACACGUUGUGGUACAUUACCUGGUA | 12386 |
| 309 | GCUAGCU GUC GCUCUGUG | 2975 | CACAGAGC AGAA GCUAGC ACCAGAGAAACACACGUUGUGGUACAUUACCUGGUA | 12387 |
| 342 | CCGAGCC GCC UCUGUGGG | 2976 | CCCACAGA AGAA GCUCGG ACCAGAGAAACACACGUUGUGGUACAUUACCUGGUA | 12388 |
| 434 | CCCUUCA GAU UACUUGCA | 2977 | UGCAAGUA AGAA GAACGG ACCACAGAAACACACGUUGUGGUACAUUACCUCGUA | 12389 |
| 630 | CUCCACU GUU UAUGUCUA | 2978 | UAGACAUA AGAA GUGGAG ACCAGAGAAACACACGUUGUGGUACAUUACCUCCUA | 12390 |
| 655 | GACUACA GAU CACCAUUC | 2979 | GAAUGGUG AGAA GUAAUC ACCAGAGAAACACACGUUGUGGUACAUUACCUCGUA | 12391 |
| 739 | AUCCCCU GCC GAGGGUCG | 2980 | CGACCCUC AGAA GGGGAU ACCAGAGAAACACACGUUGUGGUACAUUACCUGGUA | 12392 |
| 807 | UCUUCCG GAU GGAAACAG | 2981 | CUCUUUCC AGAA GCAACA ACCAGAGAAACACACGUUGUCGUACAUUACCUGGUA | 12393 |
| 920 | CCUAUCA CUC UAUCAUGU | 2982 | ACAUCAUA ACAA GAUAGG ACCAGAGAAACACACGUUGUGGUACAUUACCUCGUA | 12394 |
| 1002 | CCUAUCU CCC GCAGAAAA | 2983 | UUUUCUCC ACAA GAUAGC ACCAGAGAAACACACGUUGUGGUACAUUACCUCGUA | 12395 |
| 1229 | GUGGACG GAU GAUCAAGA | 2984 | UCUUCAUC ACAA GUCCAC ACCAGAGAAACACACGUUGUGGUACAUUACCUGGUA | 12396 |
| 1365 | UUACCCA GCU CCUGAUAU | 2985 | AUAUCAGG AGAA GGGUAA ACCACAGAAACACACCUUGUGGUACAUUACCUGGUA | 12397 |
| 1556 | CACCCCA CAU CGGUGAGA | 2986 | UCUCACCG AGAA CGCGUG ACCAGAGAAACACACCUUCUCGUACAUUACCUGGUA | 12398 |
| 1629 | AUGCACA GUC UACGCCAA | 2987 | UUGGCGUA AGAA CUCCAU ACCAGAGAAACACACCUUGUCGUACAUUACCUCGUA | 12399 |
| 1687 | CAAGCCU GCU CCUACACA | 2988 | UCUGUAGG ACAA GGCUUC ACCACAGAAACACACCUUGUCGUACAUUACCUGGUA | 12400 |
| 1696 | UCCUACA GAC CCGGCCAA | 2989 | UUGCCCGG ACAA GUAGGA ACCACACAAACACACCUUGUCGUACAUUACCUGGUA | 12401 |
| 1796 | AUCCCCU GAU UGAAGGAA | 2990 | UUCCUUCA ACAA GGGCAU ACCACACAAACACACCUUGUGCUACAUUACCUGCUA | 12402 |
| 1950 | CCAACCU GCU CCCCAGCC | 2991 | GGCUGGGC AGAA GGUUGC ACCAGAGAAACACACGUUGUGGUACAUUACCUGCUA | 12403 |
| 1953 | ACCUGCU CCC CAGCCAAC | 2992 | GUUCGCUG ACAA GCAGCU ACCAGACAAACACACGUUGUGCUACAUUACCUGCUA | 12404 |
| 1985 | UGUCCCU CUU GUCCACUG | 2993 | CAGUGCAC AGAA CGGACA ACCAGACAAACACACGUUGUGCUACAUUACCUGGUA | 12405 |
| 2055 | AACAUCG GUC CACAUGGG | 2994 | CCCAUGUC AGAA GAUGUU ACCAGAGAAACACACGUUGUGCUACAUUACCUGGUA | 12406 |
| 2082 | CACACCA GUU UGCAAGAA | 2995 | UUCUUCCA AGAA GGUGUG ACCACACAAACACACCUUGUGCUACAUUACCUGGUA | 11606 |
| 2208 | UUGCUCU GCU CAAGAUAA | 2996 | UUAUCUUG AGAA GAGCAA ACCAGAGAAACACACGUUGUGGUACAUUACCUGGUA | 12407 |
| 2252 | UCAAACA GOU CAUCAUCC | 2997 | GGAUGAUG AGAA GUUUGA ACCAGAGAAACACACGUUGUGGUACAUUACCUGGUA | 12408 |
| 2444 | CGAACCU GAC UAUCCGCA | 2998 | UGCGGAUA AGAA GGUUCC ACCAGAGAAACACACGUUGUGGUACAUUACCUCGUA | 12409 |
| 2639 | UCCUACG GAC CGUUAAGC | 2999 | GCUUAACG AGAA GUAGGA ACCAGAGAAACACACGUUGUGGUACAUUACCUGGUA | 12410 |
| 2703 | GGAUCCA GAU GAAUUGCC | 3000 | GGCAAUUC AGAA GGAUCC ACCAGAGAAACACACGUUGUGGUACAUUACCUGGUA | 12411 |
| 2777 | GGGACCG GCU GAAACUAG | 3001 | CUAGUUUC AGAA GGUCCC ACCAGAGAAACACACGUUGUGGUACAUUACCUGGUA | 12412 |
| 2832 | UGAGGCA GAC GCUUUUGG | 3002 | CCAAAAGC AGAA GCCUCA ACCAGAGAAACACACGUUGUGGUACAUUACCUGGUA | 12413 |
| 3199 | UCUGCCA GCU CAGGCUUU | 3003 | AAAGCCUG AGAA GGCAGA ACCAGAGAAACACACGUUGUGGUACAUUACCUGGUA | 12414 |
| 3278 | ACUUCCU GAC CUUGGAGC | 2199 | GCUCCAAG AGAA GGAAGU ACCAGAGAAACACACGUUGUGGUACAUUACCUGGUA | 11618 |
| 3304 | UGUUACA GCU UCCAAGUG | 2200 | CACUUGGA AGAA GUAACA ACCAGAGAAACACACGUUGUGGUACAUUACCUGGUA | 11619 |
| 3421 | GACUUCG GCU UGGCCCGG | 3004 | CCGGGCCA AGAA GAAGUC ACCAGAGAAACACACGUUGUGGUACAUUACCUGGUA | 12415 |
| 3450 | AGACCCG GAU UAUGUCAG | 3005 | CUGACAUA AGAA GGGUCU ACCAGAGAAACACACGUUGUGGUACAUUACCUGGUA | 12416 |
| 3475 | GAUGCCC GAC UCCCUUUG | 3006 | CAAAGGGA AGAA GGCAUC ACCAGAGAAACACACGUUGUGGUACAUUACCUGGUA | 12417 |
| 3663 | GGCUCCU GAC UACACUAC | 3007 | GUAGUGUA AGAA GGAGCC ACCAGAGAAACACACGUUGUGGUACAUUACCUGGUA | 12418 |
| 3689 | UGUACCA GAC CAUGCUGG | 2203 | CCAGCAUG AGAA GGUACA ACCAGAGAAACACACGUUGUGGUACAUUACCUGGUA | 11622 |
| 3703 | CUGGACU GCU GGCAUGAG | 3008 | CUCAUGCC AGAA GUCCAG ACCAGAGAAACACACGUUGUGGUACAUUACCUGGUA | 12419 |
| 3860 | UCUCCCU GCC UACCUCAC | 3009 | GUGAGGUA AGAA GGGAGA ACCAGAGAAACACACGUUGUGGUACAUUACCUGGUA | 12420 |
| 3873 | CUCACCU GUU UCCUGUAU | 2206 | AUACAGGA AGAA GGUGAG ACCAGAGAAACACACGUUGUGGUACAUUACCUGGUA | 11625 |
| 4038 | GAUCCCA GAU GACAGCCA | 3010 | UGGCUGUC AGAA GGGAUC ACCAGAGAAACACACGUUGUGGUACAUUACCUGGUA | 12421 |
| 4181 | CCAACCA GAC CAGUGGCU | 3011 | AGCCACUG AGAA GGUUGG ACCAGAGAAACACACGUUGUGGUACAUUACCUGGUA | 12422 |
| 4196 | GCUACCA GUC UGGGUAUC | 3012 | GAUACCCA AGAA GGUAGC ACCAGAGAAACACACGUUGUGGUACAUUACCUGGUA | 12423 |
| 4212 | UCACUCA GAU GACACAGA | 3013 | UCUGUGUC AGAA GAGUGA ACCAGAGAAACACACGUUGUGGUACAUUACCUGGUA | 11630 |
| 4278 | UGCUGCA GUU CACGCUGA | 3014 | UCAGCGUG AGAA GCAGCA ACCAGAGAAACACACGUUGUGGUACAUUACCUGGUA | 12424 |
| 4287 | UCACGCU GAC UCAGGGAC | 3015 | GUCCCUGA AGAA GCGUGA ACCAGAGAAACACACGUUGUGGUACAUUACCUGGUA | 12425 |
| 4307 | CACUGCA GCU CACCUCCU | 3016 | AGGAGGUG AGAA GCAGUG ACCAGAGAAACACACGUUGUGGUACAUUACCUGGUA | 12426 |
| 4318 | ACCUCCU GUU UAAAUGGA | 3017 | UCCAUUUA AGAA GGAGGU ACCAGAGAAACACACGUUGUGGUACAUUACCUGGUA | 12427 |
| 4338 | UGGUCCU GUC CCGGCUCC | 3018 | GGAGCCGG AGAA GGACCA ACCAGAGAAACACACGUUGUGGUACAUUACCUGGUA | 12428 |
| 4344 | UGUCCCG GCU CCGCCCCC | 3019 | GGGGGCGG AGAA GGGACA ACCAGAGAAACACACGUUGUGGUACAUUACCUGGUA | 12429 |
| 4349 | CGGCUCC GCC CCCAACUC | 3020 | GAGUUGGG AGAA GAGCCG ACCAGAGAAACACACGUUGUGGUACAUUACCUGGUA | 12430 |
| 4383 | AGGUGCU GCU UAGAUUUU | 3021 | AAAAUCUA AGAA GCACCU ACCAGAGAAACACACGUUGUGGUACAUUACCUGGUA | 12431 |
| 4462 | GACCUCA GAC UGCAAGGA | 3022 | UCCUUGCA AGAA GAGGUC ACCAGAGAAACACACGUUGUGGUACAUUACCUGGUA | 12432 |
| 4574 | GUGCCCU GCU GUGGUCUC | 3023 | GAGACCAC AGAA GGGCAC ACCAGAGAAACACACGUUGUGGUACAUUACCUGGUA | 12433 |
| 4626 | GGACUCU GUC CUCCAAGA | 3024 | UCUUGGAG AGAA GAGUCC ACCAGAGAAACACACGUUGUGGUACAUUACCUGGUA | 12434 |
| 4723 | CGAGUCU GUC UACCUUGG | 3025 | CCAAGGUA AGAA GACUCG ACCAGAGAAACACACGUUGUGGUACAUUACCUGGUA | 12435 |
| 4823 | GAGAGCG GUU GGAGCCUG | 3026 | CAGGCUCC AGAA GCUCUC ACCAGAGAAACACACGUUGUGGUACAUUACCUGGUA | 12436 |
| 4836 | GCCUGCA GAU GCAUUGUG | 3027 | CACAAUGC AGAA GCAGGC ACCAGAGAAACACACGUUGUGGUACAUUACCUGGUA | 12437 |
| 4896 | AAAGGCG GCC GGCAGGGU | 3028 | ACCCUGCC AGAA GCCUUU ACCAGAGAAACACACGUUGUGGUACAUUACCUGUUA | 12438 |
| 4938 | CUUCACA GUC GGGUUACA | 3029 | UGUAACCC AGAA GUGAAG ACCAGAGAAACACACGUUGUGGUACAUUACCUGGUA | 12439 |
| 4996 | CCUUCCG GAC UCUUACGU | 3030 | ACGUAAGA AGAA GGAAGG ACCAGAGAAACACACGUUGUGCUACAUUACCUGGUA | 12440 |
| 5042 | UGAUGCA GCU UGCUCCUU | 3031 | AAGGAGCA AGAA GCAUCA ACCAGAGAAACACACGUUGUGGUACAUUACCUGGUA | 12441 |
| 5118 | GGCUCCU GAC GGGGCCGA | 3032 | UCGCCCCC AGAA GGAGCC ACCAGAGAAACACACGUUGUGGUACAUUACCUGGUA | 12442 |
| 5165 | GGUUUCU GCU GGGUGCAG | 3033 | CUCCACCC AGAA GAAACC ACCAGACAAACACACGUUGUGGUACAUUACCUGGUA | 12443 |
| 5310 | GCUUCCU GAU GGCAGAAA | 3034 | UUUCUGCC AGAA GGAAGC ACCAGAGAAACACACGUUGUGGUACAUUACCUGGUA | 12444 |
| 5363 | CUAGGCA GAU UUCGAAAU | 3035 | AUUUCGAA AGAA GGCUAG ACCAGAGAAACACACGUUGUGGUACAUUACCUGGUA | 12445 |
| 5453 | GGUUUCU CCC UGUGUGCU | 3036 | ACCACACA AGAA GAAACC ACCAGAGAAACACACGUUGUGGUACAUUACCUGGUA | 12446 |

TABLE VIII
Mouse flt-1 VEGF Receptor—Hammerhead Ribozyme and Substrate Sequence

| Pos | Substrate | Seq ID No | HH Ribozyme Sequence | Seq ID No |
|---|---|---|---|---|
| 17 | GGCCGCGUC UUGCUCAC | 3037 | GUGAGCAA CUGAUGAGGCCGUUAGGCCGAA ACGCGGCC | 12447 |
| 19 | CCGCGUCUU GCUCACCA | 3038 | UGGUGAGC CUGAUGAGGCCGUUAGGCCGAA AGACGCGG | 12448 |
| 23 | GUCUUGCUC ACCAUGGU | 3039 | ACCAUGGU CUGAUGAGGCCGUUAGGCCGAA AGCAAGAC | 12449 |
| 32 | ACCAUGGUC AGCUGCUG | 3040 | CAGCAGCU CUGAUGAGGCCGUUAGGCCGAA ACCAUGGU | 12450 |
| 53 | ACCGCGGUC UUGCCUUA | 3041 | UAAGGCAA CUGAUGAGGCCGUUAGGCCGAA ACCGCGGU | 12451 |
| 55 | CGCGGUCUU GCCUUACG | 3042 | CGUAAGGC CUGAUGAGGCCGUUAGGCCGAA AGACCGCG | 12452 |
| 60 | UCUUGCCUU ACGCGCUG | 3043 | CAGCGCGU CUGAUGAGGCCGUUAGGCCGAA AGGCAAGA | 12453 |
| 61 | CUUGCCUUA CGCGCUGC | 3044 | GCAGCGCG CUGAUGAGGCCGUUAGGCCGAA AAGGCAAG | 12454 |
| 71 | GCGCUGCUC GGGUGUCU | 3045 | AGACACCC CUGAUGAGGCCGUUAGGCCGAA AGCAGCGC | 12455 |
| 78 | UCGGGUGUC UGCUUCUC | 3046 | GAGAAGCA CUGAUGAGGCCGUUAGGCCGAA ACACCCGA | 12456 |
| 83 | UGUCUGCUU CUCACAGG | 25 | CCUGUGAG CUGAUGAGGCCGUUAGGCCGAA AGCAGACA | 9444 |
| 84 | GUCUGCUUC UCACAGGA | 26 | UCCUGUGA CUGAUGAGGCCGUUAGGCCGAA AAGCAGAC | 9445 |
| 86 | CUGCUUCUC ACAGGAUA | 3047 | UAUCCUGU CUGAUGAGGCCGUUAGGCCGAA AGAAGCAG | 12457 |
| 94 | CACAGGAUA UGGCUCAG | 3048 | CUGAGCCA CUGAUGAGGCCGUUAGGCCGAA AUCCUGUG | 12458 |
| 100 | AUAUGGCUC AGGGUCGA | 3049 | UCGACCCU CUGAUGAGGCCGUUAGGCCGAA AGCCAUAU | 12459 |
| 106 | CUCAGGGUC GAAGUUAA | 3050 | UUAACUUC CUGAUGAGGCCGUUAGGCCGAA ACCCUGAG | 12460 |
| 112 | GUCGAAGUU AAAAGUGC | 3051 | GCACUUUU CUGAUGAGGCCGUUAGGCCGAA ACUUCGAC | 12461 |
| 113 | UCGAAGUUA AAAGUGCC | 3052 | GGCACUUU CUGAUGAGGCCGUUAGGCCGAA AACUUCGA | 12462 |
| 132 | AACUGAGUU UAAAAGGC | 37 | GCCUUUUA CUGAUGAGGCCGUUAGGCCGAA ACUCAGUU | 9456 |
| 133 | ACUGAGUUU AAAAGGCA | 38 | UGCCUUUU CUGAUGAGGCCGUUAGGCCGAA AACUCAGU | 9457 |
| 134 | CUGAGUUUA AAAGGCAC | 39 | GUGCCUUU CUGAUGAGGCCGUUAGGCCGAA AAACUCAG | 9458 |
| 152 | CAGCAUGUC AUGCAAGC | 3053 | GCUUGCAU CUGAUGAGGCCGUUAGGCCGAA ACAUGCUG | 12463 |
| 171 | GCCAGACUC UCUUUCUC | 3054 | GAGAAAGA CUGAUGAGGCCGUUAGGCCGAA AGUCUGGC | 12464 |
| 173 | CAGACUCUC UUUCUCAA | 3055 | UUGAGAAA CUGAUGAGGCCGUUAGGCCGAA AGAGUCUG | 12465 |
| 175 | GACUCUCUU UCUCAAGU | 3056 | ACUUGAGA CUGAUGAGGCCGUUAGGCCGAA AGAGAGUC | 12466 |
| 176 | ACUCUCUUU CUCAAGUG | 3057 | CACUUGAG CUGAUGAGGCCGUUAGGCCGAA AAGAGAGU | 12467 |
| 177 | CUCUCUUUC UCAAGUGC | 3058 | GCACUUGA CUGAUGAGGCCGUUAGGCCGAA AAAGAGAG | 12468 |
| 179 | CUCUUUCUC AAGUGCAG | 3059 | CUGCACUU CUGAUGAGGCCGUUAGGCCGAA AGAAAGAG | 12469 |
| 205 | AGCCCACUC AUGGUCUC | 3060 | GAGACCAU CUGAUGAGGCCGUUAGGCCGAA AGUGGGCU | 12470 |
| 211 | CUCAUGGUC UCUGCCCA | 3061 | UGGGCAGA CUGAUGAGGCCGUUAGGCCGAA ACCAUGAG | 12471 |
| 213 | CAUGGUCUC UGCCCACG | 3062 | CGUGGGCA CUGAUGAGGCCGUUAGGCCGAA AGACCAUG | 12472 |
| 254 | CUGAGCAUC ACUCCCCC | 3063 | GGGGGAGU CUGAUGAGGCCGUUAGGCCGAA AUGCUCAG | 12473 |
| 258 | GCAUCACUC CCCCAUCG | 3064 | CGAUGGGG CUGAUGAGGCCGUUAGGCCGAA AGUGAUGC | 12474 |
| 265 | UCCCCCAUC GGCCUGUG | 3065 | CACAGGCC CUGAUGAGGCCGUUAGGCCGAA AUGGOGGA | 12475 |
| 282 | GGAGGGAUA ACAGGCAA | 3066 | UUGCCUGU CUGAUGAGGCCGUUAGGCCGAA AUCCCUCC | 12476 |
| 292 | CAGGCAAUU CUGCAGCA | 3067 | UGCUGCAG CUGAUGAGGCCGUUAGGCCGAA AUUGCCUG | 12477 |
| 293 | AGGCAAUUC UGCAGCAC | 3068 | GUGCUGCA CUGAUGAGGCCGUUAGGCCGAA AAUUGCCU | 12478 |
| 304 | CAGCACCUU GACCUUGG | 3069 | CCAAGGUC CUGAUGAGGCCGUUAGGCCGAA AGGUGCUG | 12479 |
| 310 | CUUGACCUU GGACACGG | 3070 | CCGUGUCC CUGAUGAGGCCGUUAGGCCGAA AGGUCAAG | 12480 |
| 341 | ACGGGCCUC UACACCUG | 3071 | CAGGUGUA CUGAUGAGGCCGUUAGGCCGAA AGGCCCGU | 12481 |
| 343 | GGGCCUCUA CACCUGUA | 3072 | UACAGGUG CUGAUGAGGCCGUUAGGCCGAA AGAGGCCC | 12482 |
| 351 | ACACCUGUA GAUACCUC | 3073 | GAGGUAUC CUGAUGAGGCCGUUAGGCCGAA ACAGGUGU | 12483 |
| 355 | CUGUAGAUA CCUCCCUA | 3074 | UAGGGAGG CUGAUGAGGCCGUUAGGCCGAA AUCUACAG | 12484 |
| 359 | AGAUACCUC CCUACAUC | 3075 | GAUGUAGG CUGAUGAGGCCGUUAGGCCGAA AGGUAUCU | 12485 |
| 363 | ACCUCCCUA CAUCUACU | 3076 | AGUAGAUG CUGAUGAGGCCGUUAGGCCGAA AGGGAGGU | 12486 |
| 367 | CCCUACAUC UACUUCGA | 3077 | UCGAAGUA CUGAUGAGGCCGUUAGGCCGAA AUGUAGGG | 12487 |
| 369 | CUACAUCHA CUUCGAAG | 3078 | CUUCGAAC CUGAUGAGGCCGUUAGGCCGAA AGAUGUAG | 12488 |
| 372 | CAUCUACUC CGAAGAAA | 3079 | UUUCUUCG CUGAUGAGGCCGUUAGGCCGAA AGUAGAUG | 12489 |
| 373 | AUCUACUUC GAAGAAAA | 3080 | UUUUCUUC CUGAUGAGGCCGUUAGGCCGAA AAGUAGAU | 12490 |
| 394 | AGCGGAAUC UUCAAUCU | 3081 | AGAUUGAA CUGAUGAGGCCGUUAGGCCGAA AUUCCGCU | 12491 |
| 396 | CGGAAUCUU CAAUCUAC | 3082 | GUAGAUUG CUGAUGAGGCCGUUAGGCCGAA AGAUUCCG | 12492 |
| 397 | GGAAUCUUC AAUCUACA | 3083 | UGUAGAUU CUGAUGAGGCCGUUAGGCCGAA AAGAUUCC | 12493 |
| 401 | UCUUCAAUC UACAUAUU | 3084 | AAUAUGUA CUGAUGAGGCCGUUAGGCCGAA AUUGAAGA | 12494 |
| 403 | UUCAAUCUA CAUAUUUG | 3085 | CAAAUAUG CUGAUGAGGCCGUUAGGCCGAA AGAUUGAA | 12495 |
| 407 | AUCUACAUA UUUGUUAG | 3086 | CUAACAAA CUGAUGAGGCCGUUAGGCCGAA AUGUAGAU | 12496 |
| 409 | CUACAUAUU UGUUAGUG | 3087 | CACUAACA CUGAUGAGGCCGUUAGGCCGAA AUAUGUAG | 12497 |
| 410 | UACAUAUUU GUUAGUGA | 3088 | UCACUAAC CUGAUGAGGCCGUUAGGCCGAA PAUAUGUA | 12498 |
| 413 | AUAUUUGUU AGUGAUGC | 3089 | GCAUCACU CUGAUGAGGCCGUUAGGCCGAA ACAAAUAU | 12499 |
| 414 | UAUUUGUUA GUGAUCCA | 3090 | UGCAUCAC CUGAUGAGGCCGUUAGGCCGAA AACAAAUA | 12500 |
| 429 | CAGGGAGUC CUUUCAUA | 3091 | UAUGAAAG CUGAUGAGGCCGUUAGGCCGAA ACUCCCUG | 12501 |
| 432 | GGAGUCCUU UCAUAGAG | 3092 | CUCUAUGA CUGAUGAGGCCGUUAGGCCGAA AGGACUCC | 12502 |
| 433 | GAGUCCUUU CAUAGAGA | 3093 | UCUCUAUG CUGAUGAGGCCGUUAGGCCGAA AAGGACUC | 12503 |
| 434 | AGUCCUUUC AUAGAGAU | 3094 | AUCUCUAU CUGAUGAGGCCGUUAGGCCGAA AAAGGACU | 12504 |
| 437 | CCUUUCAUA GAGAUGCA | 3095 | UGCAUCUC CUGAUGAGGCCGUUAGGCCGAA AUGAAAGG | 12505 |
| 455 | ACUGACAUA CCCAAACU | 3096 | AGUCUGOG CUGAUGAGGCCGUUAGGCCGAA AUGUCAGU | 12506 |
| 464 | CCCAAACUU GUGCACAU | 3097 | AUGUGCAC CUGAUGAGGCCGUUAGGCCGAA AGUUUGGG | 12507 |
| 491 | AGACAGCUC AUCAUCCC | 3098 | GGGAUGAU CUGAUGAGGCCGUUAGGCCGAA AGCUGUCU | 12508 |
| 494 | CAGCUCAUC AUCCCCUG | 3099 | CAGGGGAU CUGAUGAGGCCGUUAGGCCGAA AUGAGCUG | 12509 |
| 497 | CUCAUCAUC CCCUGCCG | 3100 | CGGCAGGG CUGAUGAGGCCGUUAGGCCGAA AUGAUGAG | 12510 |
| 514 | GGUGACGUC ACCCAACG | 3101 | CGUUGGGU CUGAUGAGGCCGUUAGGCCGAA ACGUCACC | 12511 |
| 524 | CCCAACGUC ACAGUCAC | 3102 | GUGACUGU CUGAUGAGGCCGUUAGGCCGAA ACGUUGGG | 12512 |
| 530 | GUCACAGUC ACCCUAAA | 3103 | UUUAGGGU CUGAUGAGGCCGUUAGGCCGAA ACUGUGAC | 12513 |
| 536 | GUCACCCUA AAAAAGUU | 3104 | AACUUUUU CUGAUGAGGCCGUUAGGCCGAA AGUGUGAC | 12514 |
| 544 | AAAAAAGUU UCCAUUUG | 3105 | CAAAUGGA CUGAUGAGGCCGUUAGGCCGAA ACUUUUUU | 12515 |
| 545 | AAAAAGUUU CCAUUUGA | 3106 | UCAAAUGG CUGAUGAGGCCGUUAGGCCGAA AACUUUUU | 12516 |
| 546 | AAAAGUUUC CAUUUGAU | 3107 | AUCAAAUG CUGAUGAGGCCGUUAGGCCGAA AAACUUUU | 12517 |
| 550 | GUUUCCAUU UGAHACUC | 3108 | GAGUAUCA CUGAUGAGGCCGUUAGGCCGAA AUGGAAAC | 12518 |
| 551 | UUUCCAUUU GAUACUCU | 3109 | AGAGUAUC CUGAUGAGGCCGUUAGGCCGAA AAUGGAAA | 12519 |
| 555 | CAUUUGAUA CUCUUACC | 3110 | GGUAAGAG CUGAUGAGGCCGUUAGGCCGAA AUCAAAUG | 12520 |
| 558 | UUGAUACUC UCACCCCU | 3111 | AGGGGUAA CUGAUGAGGCCGUUAGGCCGAA AGUAUCAA | 12521 |
| 560 | GAUACUCUU ACCCCUGA | 3112 | UCAGGGGU CUGAUGAGGCCGUUAGGCCGAA AGAGUAUC | 12522 |
| 561 | AUACUCUUA CCCCUGAU | 3113 | AUCAGGGG CUGAUGAGGCCGUUAGGCCGAA AAGAGUAU | 12523 |
| 581 | CAAAGAAUA ACAUGGGA | 3114 | UCCCAUGU CUGAUGAGGCCGUUAGGCCGAA AUUCUUUG | 12524 |
| 594 | GGGACAGUA GGAGAGGC | 3115 | GCCUCUCC CUGAUGAGGCCGUUAGGCCGAA ACUGUCCC | 12525 |
| 604 | GAGAGGCUU UAUAAUAG | 3116 | CUAUUAUA CUGAUGAGGCCGUUAGGCCGAA AGCCUCUC | 12526 |
| 605 | AGAGGCUUU AUAAUAGC | 3117 | GCUAUUAU CUGAUGAGGCCGUUAGGCCGAA AAGCCUCU | 12527 |
| 606 | GAGGCUUUA UAAUAGCA | 3118 | UGCUAUUA CUGAUGAGGCCGUUAGGCCGAA AAAGCCUC | 12528 |
| 608 | GGCUUUAUA AUAGCAAA | 3119 | UUUGCUAU CUGAUGAGGCCGUUAGGCCGAA AUAAAGCC | 12529 |
| 611 | UUUAUAAUA GCAAAUGC | 3120 | GCAUUUGC CUGAUGAGGCCGUUAGGCCGAA AUUAUAAA | 12530 |
| 625 | UGCAACGUA CAAAGAGA | 3121 | UCUCUUUG CUGAUGAGGCCGUUAGGCCGAA ACGUUGCA | 12531 |
| 635 | AAAGAGAUA GGACUGCU | 3122 | AGCAGUCC CUGAUGAGGCCGUUAGGCCGAA AUCUCUUU | 12532 |
| 662 | GCCACCGUC AACGGGCA | 3123 | UGCCCGUU CUGAUGAGGCCGUUAGGCCGAA ACGGUGGC | 12533 |
| 676 | GCACCUGUA CCAGACAA | 3124 | UUGUCUGG CUGAUGAGGCCGUUAGGCCGAA ACAGGUGC | 12534 |
| 688 | GACAAACUA UCUGACCC | 3125 | GGGUCAGA CUGAUGAGGCCGUUAGGCCGAA AGUCUGUC | 12535 |
| 690 | CAAACUAUC UGACCCAU | 3126 | AUGGGUCA CUGAUGAGGCCGUUAGGCCGAA AUAGUUUG | 12536 |
| 699 | UGACCCAUC GGCAGACC | 3127 | GGUCUGCC CUGAUGAGGCCGUUAGGCCGAA AUGGGUCA | 12537 |
| 711 | AGACCAAUA CAAUCCUA | 3128 | UAGGAUUG CUGAUGAGGCCGUUAGGCCGAA AUUGGUCU | 12538 |
| 716 | AAUACAAUC CUAGAUGU | 3129 | ACAUCUAG CUGAUGAGGCCGUUAGGCCGAA AUUGUAUU | 12539 |
| 719 | ACAAUCCUA GAUGUCCA | 3130 | UGGACAUC CUGAUGAGGCCGUUAGGCCGAA AGGAUUGU | 12540 |
| 725 | CUAGAUGUC CAAAUACG | 3131 | CGUAUUUG CUGAUGAGGCCGUUAGGCCGAA ACAUCUAG | 12541 |
| 731 | GUCCAAAUA CGCCCGCC | 3132 | GGCGGGCG CUGAUGAGGCCGUUAGGCCGAA AUUUGGAC | 12542 |
| 758 | AGACUOCUC CACGGGCA | 3133 | UGCCCGUG CUGAUGAGGCCGUUAGGCCGAA AGCAGUCU | 12543 |
| 771 | GUCAGACUC UGGUCCUC | 3134 | GAGGACAA CUGAUGAGGCCGUUAGGCCGAA AGUCUGCC | 12544 |
| 773 | CAGACUCUU GUCCUCAA | 3135 | GUGAGGAC CUGAUGAGGCCGUUAGGCCGAA AGAGUCUG | 12545 |
| 776 | ACUCUGGUC CUCAACUG | 3136 | CAGUGGAG CUGAUGAGGCCGUUAGGCCGAA ACAAGAGU | 12546 |
| 779 | CUUGUCCUC AACUGCAC | 3137 | GUGCAGUU CUGAUGAGGCCGUUAGGCCGAA AGGACAAG | 12547 |
| 803 | ACGGAGCUC AAUACGAG | 3138 | CUCGUAUU CUGAUGAGGCCGUUAGGCCGAA AGCUCCGU | 12548 |
| 807 | AGCUCAAUA CGAGGGUG | 3139 | CACCCUCG CUGAUGAGGCCGUUAGGCCGAA AUUGAGCU | 12549 |
| 831 | GCUGGAAUU ACCCUGGU | 3140 | ACCAGGGU CUGAUGAGGCCGUUAGGCCGAA AUUCCAGC | 12550 |
| 832 | CUGGAAUUA CCCUGGUA | 3141 | UACCAGGG CUGAUGAGGCCGUUAGGCCGAA AAUUCCAG | 12551 |
| 840 | ACCCUGGUA AAGCAACU | 3142 | AGUUGCUU CUGAUGAGGCCGUUAGGCCGAA ACCAGGGU | 12552 |
| 849 | AAGCAACUA AGAGAGCA | 3143 | UGCUCUCU CUGAUGAGGCCGUUAGGCCGAA AGUUGCUU | 12553 |
| 859 | GAGAGCAUC UAUAAGGC | 3144 | GCCUUAUA CUGAUGAGGCCGUUAGGCCGAA AUCCUCUC | 12554 |
| 861 | GAGCAUCUA UAAGGCAG | 3145 | CUGCCUUA CUGAUGAGGCCGUUAGGCCGAA AGAUGCUC | 12555 |
| 863 | GCAUCUAUA AGGCAGCG | 3146 | CGCUGCCU CUGAUGAGGCCGUUAGGCCGAA AUAGAUGC | 12556 |
| 875 | CAGCGGAUU GACCGGAG | 3147 | CUCCGGUC CUGAUGAGGCCGUUAGGCCGAA AUCCGCUG | 12557 |
| 888 | GGAGCCAUU CCCACAAC | 3148 | GUUGUGGG CUGAUGAGGCCGUUAGGCCGAA AUGGCUCC | 12558 |
| 889 | GAGCCAUUC CCACAACA | 3149 | UGUUGUGG CUGAUGAGGCCGUUAGGCCGAA AAUGGCUC | 12559 |
| 904 | CAAUGUGUU CCACAGUG | 3150 | CACUGUGG CUGAUGAGGCCGUUAGGCCGAA ACACAUUG | 12560 |
| 905 | AAUGUGUGC CACAGUGU | 3151 | ACACUGUG CUGAUGAGGCCGUGAGGCCGAA AACACAUG | 12561 |
| 914 | CACAGUGUG CUGAAGAU | 3152 | AUCUGAAG CUGAUGAGGCCGUGAGGCCGAA ACACUGUG | 12562 |
| 915 | ACAGUGUGC UGAAGAUC | 3153 | GAUCUGAA CUGAUGAGGCCGUGAGGCCGAA AACACUGU | 12563 |
| 917 | AGUGUGCUG AAGAUCAA | 3154 | UGGAUCUG CUGAUGAGGCCGUGAGGCCGAA AGAACACU | 12564 |
| 918 | GUGUGCUGA AGAUCAAC | 3155 | GUGGAUCU CUGAUGAGGCCGUGAGGCCGAA AAGAACAC | 12565 |
| 923 | CUGAAGAUC AACAAUGU | 3156 | ACAUGGUG CUGAUGAGGCCGUGAGGCCGAA AUCUGAAG | 12566 |
| 953 | AAGGGGCUC UACACCUG | 3157 | CAGGUGUA CUGAUGAGGCCGUGAGGCCGAA AGCCCCUG | 12567 |
| 955 | GGGGCUCUA CACCUGUC | 3158 | GACAGGUG CUGAUGAGGCCGUGAGGCCGAA AGAGCCCC | 12568 |
| 963 | ACACCUGUC GCGUGAAG | 3159 | CUGCACGC CUGAUGAGGCCGUGAGGCCGAA ACAGGUGU | 12569 |
| 979 | GAGUGGGUC CUCGUGCC | 3160 | GGAACGAG CUGAUGAGGCCGUGAGGCCGAA ACCCACUC | 12570 |
| 982 | UGGGUCCUC GUGCCAGU | 3161 | ACUGGAAC CUGAUGAGGCCGUGAGGCCGAA AGGACCCA | 12571 |
| 985 | GUCCUCGUG CCAGUCUG | 3162 | AAGACUGG CUGAUGAGGCCGUGAGGCCGAA ACGAGGAC | 12572 |
| 986 | UCCUCGUGC CAGUCUUU | 3163 | AAAGACUG CUGAUGAGGCCGUGAGGCCGAA AACGAGGA | 12573 |
| 991 | GUGCCAGUC UUUCAACA | 3164 | UGUGGAAA CUGAUGAGGCCGUGAGGCCGAA ACUGGAAC | 12574 |
| 993 | UCCAGUCUG UCAACACC | 3165 | GGUGUGGA CUGAUGAGGCCGUGAGGCCGAA AGACUGGA | 12575 |
| 994 | CCAGUCUUU CAACACCU | 3166 | AGGUGUGG CUGAUGAGGCCGUGAGGCCGAA AAGACUGG | 12576 |
| 995 | CAGUCUGUC AACACCUC | 3167 | GAGGUGUG CUGAUGAGGCCGUGAGGCCGAA AAAGACUG | 12577 |
| 1003 | CAACACCUC CGUGCAUG | 3168 | CAUGCACG CUGAUGAGGCCGUUAGGCCGAA AGGUGUGG | 12578 |
| 1015 | GCAUGUGUA UGAAAAAG | 3169 | CUUUDUCA CUGAUGAGGCCGUUAGGCCGAA ACACAUGC | 12579 |
| 1027 | AAAAGGAUG CAUCAGUG | 3170 | CACUGAUG CUGAUGAGGCCGUUAGGCCGAA AUCCUUUU | 12580 |
| 1028 | AAAGGAUGC AUCAGUGU | 3171 | ACACUGAU CUGAUGAGGCCGUUAGGCCGAA AAUCCUUU | 12581 |
| 1031 | GGAUGCAUC AGUGUGAA | 3172 | UGCACACU CUGAUGAGGCCGUUAGGCCGAA AUGAAUCC | 12582 |
| 1044 | UGAAACAUC GGAAGCAG | 3173 | CUGCUGCC CUGAUGAGGCCGUUAGGCCGAA AUGUUUCA | 12583 |
| 1084 | AAGACGGUC CUAUCGGC | 3174 | GCCGAUAG CUGAUGAGGCCGUUAGGCCGAA ACCGUCUG | 12584 |
| 1087 | ACGGUCCUA UCGGCUGU | 3175 | ACAGCCGA CUGAUGAGGCCGUUAGGCCGAA AGGACCGU | 12585 |
| 1089 | GGUCCUAUC GGCUGUCC | 3176 | GGACAGCC CUGAUGAGGCCGUUAGGCCGAA AUAGGACC | 12586 |
| 1096 | UCGGCUGUC CAUGAAAG | 3177 | CUUUCAUG CUGAUGAGGCCGUUAGGCCGPA ACAGCCGA | 12587 |
| 1114 | GAAGGCCUG CCCCUCCC | 3178 | GGGAGGGG CUGAUGAGGCCGUUAGGCCGAA AGGCCUGC | 12588 |
| 1115 | AAGGCCUUC CCCUCCCC | 3179 | GGGGAGGG CUGAUGAGGCCGUUAGGCCGAA AAGGCCUU | 12589 |
| 1120 | CUUCCCCUC CCCAGAAA | 3180 | UUUCUGGG CUGAUGAGGCCGUUAGGCCGAA AGGGGAAG | 12590 |
| 1130 | CCAGAAAUC GUAUGGUU | 3181 | AACCAUAC CUGAUGAGGCCGUUAGGCCGAA AUUUCUGG | 12591 |
| 1133 | GAAAUCGUA UGGUUAAA | 3182 | UUUAACCA CUCAUGAGGCCGUUAGGCCCAA ACGAUUUC | 12592 |
| 1138 | CUUAUUGUU AAAACAUG | 3183 | CAUCUUUU CUGAUGAGGCCGUUAGGCCGAA ACCAUACG | 12593 |
| 1139 | GUAUGGUUA AAAGAUGG | 210 | CCAUCUUU CUGAUGAGGCCGUUAGGCCGAA AACCAUAC | 9629 |
| 1150 | AGAUGGCUC GCCUGCAA | 3184 | UUGCAGGC CUGAUGAGGCCGUUAGGCCGAA AGCCAUCU | 12594 |
| 1162 | UGCAACAUU GAAGUCUG | 3185 | CAGACUUC CUGAUGAGGCCGUUAGGCCGAA AUGUUGCA | 12595 |
| 1168 | AUUGAAGUC UGCUCGCU | 3186 | AGCGAGCA CUGAUGAGGCCGUUAGGCCGAA ACUUCAAU | 12596 |
| 1173 | AGUCUGCUC GCUAUUUG | 3187 | CAAAUAGC CUGAUGAGGCCGUUAGGCCGAA AGCAGACU | 12597 |
| 1177 | UGCUCGCUA UUUGGUAC | 3188 | GUACCAAA CUGAUCAGGCCGUUAGGCCGAA AGCCAGCA | 12598 |
| 1179 | CUCGCUAUU UGGUACAU | 3189 | AUGUACCA CUGAUGAGGCCGUUAGGCCGAA AUAGCGAG | 12599 |
| 1180 | UCGCUAUUU GGUACAUG | 3190 | CAUGUACC CUGAUGAGGCCGUUAGGCCGAA AAUAGCGA | 12600 |
| 1184 | UAUUUGGUA CAUGGCUA | 3191 | UAGCCAUG CUGAUGAGGCCGUUAGGCCGAA ACCAAAUA | 12601 |
| 1192 | ACAUGGCUA CUCAUUAA | 3192 | UUAAUGAG CUGAUGAGGCCGUUAGGCCGAA AGCCAUGU | 12602 |
| 1195 | UGGCUACUC AUUAAUUA | 3193 | UAAUUAAU CUGAUGAGGCCGUUAGGCCGAA AGUAGCCA | 12603 |
| 1198 | CUACUCAUU AAUUAUCA | 3194 | UGAUAAUU CUGAUGAGGCCGUUAGGCCGAA AUGAGUAG | 12604 |
| 1199 | UACUCAUUA AUUAUCAA | 3195 | UUGAUAAU CUGAUGAGGCCGUUAGGCCGAA AAUGAGUA | 12605 |
| 1202 | UCAUUAAUU AUCAAAGA | 3196 | UCUUUGAU CUGAUGAGGCCGUUAGGCCGAA AUUAAUGA | 12606 |
| 1203 | CAUUAAUUA UCAAAGAU | 3197 | AUCUUUGA CUGAUGAGGCCGUUAGGCCGAA AAUUAAUG | 12607 |
| 1205 | UUAAUUAUC AAAGAUGU | 3198 | ACAUCUUU CUGAUGAGGCCGUUAGGCCGAA AUAAUUAA | 12608 |
| 1237 | AGGGGACUA UACGAUCU | 3199 | AGAUCGUA CUGAUGAGGCCGUUAGGCCGAA AGUCCCCU | 12609 |
| 1239 | GGGACUAUA CGAUCUUG | 3200 | CAAGAUCG CUGAUGAGGCCGUUAGGCCGAA AUAGUCCC | 12610 |
| 1244 | UAUACGAUC UUGCUGGG | 3201 | CCCAGCAA CUGAUGAGGCCGUUAGGCCGAA AUCGUAUA | 12611 |
| 1246 | UACGAUCUU GCUGGGCA | 3202 | UGCCCAGC CUGAUGAGGCCGUUAGGCCGAA AGAUCGUA | 12612 |
| 1256 | CUGGGCAUA AAGCAGUC | 3203 | GACUGCUU CUGAUGAGGCCGUUAGGCCGAA AUGCCCAG | 12613 |
| 1264 | AAAGCAGUC AAGGCUAU | 3204 | AUAGCCUU CUGAUGAGGCCGUUAGGCCGAA ACUGCUUU | 12614 |
| 1271 | UCAAGGCUA UUUAAAAA | 3205 | UUUUUAAA CUGAUGAGGCCGUUAGGCCGAA AGCCUUGA | 12615 |
| 1273 | AAGGCUAUU UAAAAACC | 3206 | GGUUUUUA CUGAUGAGGCCGUUAGGCCGAA AUAGCCUU | 12616 |
| 1274 | AGGCUAUUU AAAAACCU | 3207 | AGGUUUUU CUGAUGAGGCCGUUAGGCCGAA AAUAGCCU | 12617 |
| 1275 | GGCUAUUUA AAAACCUC | 3208 | GAGGUUUU CUGAUGAGGCCGUUAGGCCGAA AAAUAGCC | 12618 |
| 1283 | AAAAACCUC ACUGCCAC | 237 | GUGGCAGU CUGAUGAGGCCGUUAGGCCGAA AGGUUUUU | 9656 |
| 1293 | CUGCCACUC UCAUUGUA | 3209 | UACAAUGA CUGAUGAGGCCGUUAGGCCGAA AGUGGCAG | 12619 |
| 1295 | GCCACUCUC AUUGUAAA | 3210 | UUUACAAU CUGAUGAGGCCGUUAGGCCGAA AGAGUGGC | 12620 |
| 1298 | ACUCUCAUU GUAAACGU | 3211 | ACGUUUAC CUGAUGAGGCCGUUAGGCCGAA AUGAGAGU | 12621 |
| 1301 | CUCAUUGUA AACGUGAA | 3212 | UUCACGUU CUGAUGAGGCCGUUAGGCCGAA ACAAUGAG | 12622 |
| 1314 | UGAAACCUC AGAUCUAC | 3213 | GUAGAUCU CUGAUGAGGCCGUUAGGCCGAA AGGUUUCA | 12623 |
| 1319 | CCUCAGAUC UACGAAAA | 3214 | UUUUCGUA CUGAUGAGGCCGUUAGGCCGAA AUCUGAGG | 12624 |
| 1321 | UCAGAUCUA CGAAAAGU | 3215 | ACUUUUCG CUGAUGAGGCCGUUAGGCCGAA AGAUCUGA | 12625 |
| 1330 | CGAAAAGUC CGUGUCCU | 3216 | AGGACACG CUGAUGAGGCCGUUAGGCCGAA ACUUUUCG | 12626 |
| 1336 | GUCCGUGUC CUCGCUUC | 3217 | GAAGCGAG CUGAUGAGGCCGUUAGGCCGAA ACACGGAC | 12627 |
| 1339 | CGUGUCCUC GCUUCCAA | 3218 | UUGGAAGC CUGAUGAGGCCGUUAGGCCGAA AGGACACG | 12628 |
| 1343 | UCCUCGCUU CCAAGCCC | 3219 | GGGCUUGG CUGAUGAGGCCGUUAGGCCGAA AGCGAGGA | 12629 |
| 1344 | CCUCGCUUC CAAGCCCA | 3220 | UGGGCUUG CUGAUGAGGCCGUUAGGCCGAA AAGCGAGG | 12630 |
| 1356 | GCCCACCUC UCUAUCCG | 3221 | CGGAUAGA CUGAUGAGGCCGUUAGGCCGAA AGGUGGGC | 12631 |
| 1358 | CCACCUCUC UAUCCGCU | 3222 | AGCGGAUA CUGAUGAGGCCGUUAGGCCGAA AGAGGUGG | 12632 |
| 1360 | ACCUCUCUA UCCGCUGG | 3223 | CCAGCGGA CUGAUGAGGCCGUUAGGCCGAA AGAGAGGU | 12633 |
| 1362 | CUCUCUAUC CGCUGGGC | 3224 | GCCCAGCG CUGAUGAGGCCGUUAGGCCGAA AUAGAGAG | 12634 |
| 1382 | AGACAAGUC CUCACUUG | 3225 | CAAGUGAG CUGAUGAGGCCGUUAGGCCGAA ACUUGUCU | 12635 |
| 1385 | CAAGUCCUC ACUUGCAC | 3226 | GUGCAAGU CUGAUGAGGCCGUUAGGCCGAA AGGACUUG | 12636 |
| 1389 | UCCUCACUU GCACCGUG | 3227 | CACGGUGC CUGAUGAGGCCGUUAGGCCGAA AGUGAGGA | 12637 |
| 1399 | CACCGUCUA UGGCAUCC | 3228 | GGAUGCCA CUGAUGAGGCCGUUAGGCCGAA ACACGGUG | 12638 |
| 1406 | UAUGGCAUC CCUCGGCC | 3229 | GGCCGAGG CUGAUGAGGCCGUUAGGCCGAA AUGCCAUA | 12639 |
| 1410 | GCAUCCCUC GGCCAACA | 3230 | UGUUGGCC CUGAUGAGGCCGUUAGGCCGAA AGGGAUGC | 12640 |
| 1421 | CCAACAAUC ACGUGGCU | 3231 | AGCCACGU CUGAUGAGGCCGUUAGGCCGAA AUUGUUGG | 12641 |
| 1430 | ACGUGGCUC UGGCACCC | 3232 | GGGUGCCA CUGAUGAGGCCGUUAGGCCGAA AGCCACGU | 12642 |
| 1443 | ACCCCUGUC ACCACAAU | 3233 | AUUGUGGU CUGAUGAGGCCGUUAGGCCGAA ACAGGGGU | 12643 |
| 1452 | ACCACAAUC ACUCCAAA | 3234 | UUUGGAGU CUGAUGAGGCCGUUAGGCCGAA AUUGUGGU | 12644 |
| 1456 | CAAUCACUC CAAAGAAA | 3235 | UUUCUUUG CUGAUGAGGCCGUUAGGCCGAA AGUGAUUG | 12645 |
| 1468 | AGAAAGGUA UGACUUCU | 3236 | AGAAGUCA CUGAUGAGGCCGUUAGGCCGAA ACCUUUCU | 12646 |
| 1474 | GUAUGACUU CUGCACUG | 3237 | CAGUGCAG CUGAUGAGGCCGUUAGGCCGAA AGUCAUAC | 12647 |
| 1475 | UAUGACUUC UGCACUGA | 3238 | UCAGUGCA CUGAUGAGGCCAGUUGGCCGAA AAGUCAUA | 12648 |
| 1495 | UGAAUAAUC CUUUAUCC | 3239 | GGAUAAAG CUGAUGAGGCCAGUUGGCCGAA AUUCUUCA | 12649 |
| 1498 | AGAAUCCUU UAUCCUGG | 3240 | CCAGGAUA CUGAUGAGGCCAGUUGGCCGAA AGGAUUCU | 12650 |
| 1499 | GAAUCCUUU AUCCUGGA | 3241 | UCCAGGAU CUGAUGAGGCCAGUUGGCCGAA AAGGAUUC | 12651 |
| 1500 | AAUCCUUUA UCCUGGAU | 3242 | AUCCAGGA CUGAUGAGGCCAGUUGGCCGAA AAAGGAUU | 12652 |
| 1502 | UCCUUUAUC CUGGAUCC | 3243 | GGAUCCAG CUGAUGAGGCCAGUUGGCCGAA AUAAAGGA | 12653 |
| 1509 | UCCUGGAUC CCAGCAGC | 3244 | GCUGCUGG CUGAUGAGGCCAGUUGGCCGAA AUCCAGGA | 12654 |
| 1522 | CAGCAACUU AGGAAACA | 3245 | UGUUUCCU CUGAUGAGGCCAGUUGGCCGAA AGUUGCUG | 12655 |
| 1523 | AGCAACUUA GGAAACAG | 3246 | CUGUUUCC CUGAUGAGGCCAGUUGGCCGAA AAGUUGCU | 12656 |
| 1535 | AACAGAAUU GAGAGCAU | 280 | AUGCUCUC CUGAUGAGGCCAGUUGGCCGAA AUUCUGUU | 9699 |
| 1544 | GAGAGCAUC UCUCAGCG | 3247 | CGCUGAGA CUGAUGAGGCCAGUUGGCCGAA AUGCUCUC | 12657 |
| 1546 | GAGCAUCUC UCAGCGCA | 3248 | UGCGCUGA CUGAUGAGGCCAGUUGGCCGAA AGAUCCUC | 12658 |
| 1548 | GCAUCUCUC AGCGCAUG | 3249 | CAUGCGCU CUGAUGAGGCCAGUUGGCCGAA AGAGAUGC | 12659 |
| 1562 | AUGACGGUC AUAGAAGG | 3250 | CCUUCUAU CUGAUGAGGCCAGUUGGCCGAA ACCGUCAU | 12660 |
| 1565 | ACGGUCAUA GAAGGAAC | 3251 | GUUCCUUC CUGAUGAGGCCAGUUGGCCGAA AUGACCGU | 12661 |
| 1578 | GAACAAAUA AGACGGUU | 3252 | AACCGUCU CUGAUGAGGCCAGUUGGCCGAA AUUUGUUC | 12662 |
| 1586 | AAGACGGUU AGCACAUU | 3253 | AAUGUGCU CUGAUGAGGCCAGUUGGCCGAA ACCGUCUU | 12663 |
| 1587 | AGACGGUUA GCACAUUG | 3254 | CAAUGUGC CUGAUGAGGCCAGUUGGCCGAA AACCGUCU | 12664 |
| 1594 | UAGCACAUU GGUGGUGG | 3255 | CCACCACC CUGAUGAGGCCAGUUGGCCGAA AUGUGCUA | 12665 |
| 1609 | GGCUGACUC UCAGACCC | 3256 | GGGUCUGA CUGAUGAGGCCAGUUGGCCGAA AGUCAGCC | 12666 |
| 1611 | CUGACUCUC AGACCCCU | 3257 | AGGGGUCU CUGAUGAGGCCAGUUGGCCGAA AGAGUCAG | 12667 |
| 1625 | CCUGGAAUC UACAGCUG | 3258 | CAGCUGUA CUGAUGAGGCCAGUUGGCCGAA AUUCCAGG | 12668 |
| 1627 | UGGAAUCUA CAGCUGCC | 3259 | GGCAGCUG CUGAUGAGGCCAGUUGGCCGAA AGAUUCCA | 12669 |
| 1642 | CCGGGCCUU CAAUAAAA | 3260 | UUUUAUUG CUGAUGAGGCCAGUUGGCCGAA AGGCCCGG | 12670 |
| 1643 | CGGGCCUUC AAUAAAAU | 3261 | AUUUUAUU CUGAUGAGGCCAGUUGGCCGAA AAGGCCCG | 12671 |
| 1647 | CCUUCAAUA AAAUAGGG | 3262 | CCCUAUUU CUGAUGAGGCCAGUUGGCCGAA AUUGAAGG | 12672 |
| 1652 | AAUAAAAUA GGGACUGU | 3263 | ACAGUCCC CUGAUGAGGCCAGUUGGCCGAA AUUUUAUU | 12673 |
| 1673 | AGAAACAUA AAAUUUUA | 3264 | UAAAAUUU CUGAUGAGGCCAGUUGGCCGAA AUGUUUCU | 12674 |
| 1678 | CAUAAAAUU UUAUGUCA | 3265 | UGACAUAA CUGAUGAGGCCAGUUGGCCGAA AUUUUAUG | 12675 |
| 1679 | AUAAAAUUU UAUGUCAC | 3266 | GUGACAUA CUGAUGAGGCCAGUUGGCCGAA AAUUUUAU | 12676 |
| 1680 | UAAAAUUUU AUGUCACA | 3267 | UGUGACAU CUGAUGAGGCCAGUUGGCCGAA AAAUUUUA | 12677 |
| 1681 | AAAAUUUUA UGUCACAG | 3268 | CUGUGACA CUGAUGAGGCCAGUUGGCCGAA AAAAUUUU | 12678 |
| 1685 | UUUUAUGUC ACAGAUGU | 3269 | ACAUCUGU CUGAUGAGGCCAGUUGGCCGAA ACAUAAAA | 12679 |
| 1705 | GAAUGGCUU UCACGUUU | 3270 | AAACGUGA CUGAUGAGGCCAGUUGGCCGAA AGCCAUUC | 12680 |
| 1706 | AAUGGCUUU CACGUUUC | 3271 | GAAACGUG CUGAUGAGGCCAGUUGGCCGAA AAGCCAUU | 12681 |
| 1707 | AUGGCUUUC ACGUUUCC | 3272 | GGAAACGU CUGAUGAGGCCAGUUGGCCGAA AAAGCCAU | 12682 |
| 1712 | UUUCACGUU UCCUUGGA | 3273 | UCCAAGGA CUGAUGAGGCCAGUUGGCCGAA ACGUGAAA | 12683 |
| 1713 | UUCACGUUU CCUUGGAA | 3274 | UUCCAAGG CUGAUGAGGCCAGUUGGCCGAA AACGUGAA | 12684 |
| 1714 | UCACGUUUC CUUGGAAA | 3275 | UUUCCAAG CUGAUGAGGCCAGUUGGCCGAA AAACGUGA | 12685 |
| 1717 | CGUUUCCUU GGAAAAGA | 3276 | UCUUUUCC CUGAUGAGGCCAGUUGGCCGAA AGGAAACG | 12686 |
| 1756 | GAAACUGUC CUGUGUGG | 3277 | CCACACAG CUGAUGAGGCCAGUUGGCCGAA ACAGUUUC | 12687 |
| 1766 | UGUGUGGUC AAUAAAUU | 3278 | AAUUUAUU CUGAUGAGGCCAGUUGGCCGAA ACCACACA | 12688 |
| 1770 | UGGUCAAUA AAUUCCUG | 3279 | CAGGAAUU CUGAUGAGGCCAGUUGGCCGAA AUUGACCA | 12689 |
| 1774 | CAAUAAAUU CCUGUACA | 3280 | UGUACAGG CUGAUGAGGCCAGUUGGCCGAA AUUUAUUG | 12690 |
| 1775 | AAUAAAUUC CUGUACAG | 3281 | CUGUACAG CUGAUGAGGCCAGUUGGCCGAA AAUUUAUU | 12691 |
| 1780 | AUUCCUGUA CAGAGACA | 3282 | UGUCUCUG CUGAUGAGGCCAGUUGGCCGAA ACAGGAAU | 12692 |
| 1790 | AGAGACAUU ACCUGGAU | 3283 | AUCCAGGU CUGAUGAGGCCAGUUGGCCGAA AUGUCUCU | 12693 |
| 1791 | GAGACAUUA CCUGGAUU | 3284 | AAUCCAGG CUGAUGAGGCCAGUUGGCCGAA AAUGUCUC | 12694 |
| 1799 | ACCUGGAUG CUGCUACG | 3285 | CGUAGCAG CUGAUGAGGCCAGUUGGCCGAA AUCCAGGU | 12695 |
| 1800 | CCUGGAUUC UGCUACGG | 3286 | CCGUAGCA CUGAUGAGGCCAGUUGGCCGAA AAUCCAGG | 12696 |
| 1805 | AUUCUGCUA CGGACAGU | 3287 | ACUGUCCG CUGAUGAGGCCAGUUGGCCGAA AGCAGAAU | 12697 |
| 1814 | CGGACAGUU AACAACAG | 3288 | CUGUUGUU CUGAUGAGGCCAGUUGGCCGAA ACUGUCCG | 12698 |
| 1815 | GGACAGUUA ACAACAGA | 3289 | UCUGUUGU CUGAUGAGGCCAGUUGGCCGAA AACUGUCC | 12699 |
| 1836 | UGCACCAUA GUAUCAGC | 3290 | GCUGAUAC CUGAUGAGGCCAGUUGGCCGAA AUGGUGCA | 12700 |
| 1839 | ACCAUAGUA UCAGCAAG | 3291 | CUUGCUGA CUGAUGAGGCCAGUUGGCCGAA ACUAUGGU | 12701 |
| 1841 | CAUAGUAUC AGCAAGCA | 3292 | UGCUUGCU CUGAUGAGGCCAGUUGGCCGAA AUACUAUG | 12702 |
| 1866 | CCACCACUC AAGAUUAC | 3293 | GUAAUCUU CUGAUGAGGCCAGUUGGCCGAA AGUGGUGG | 12703 |
| 1872 | CUCAAGAUU ACUCCAUC | 3294 | GAUGGAGU CUGAUGAGGCCAGUUGGCCGAA AUCUUGAG | 12704 |
| 1873 | UCAAGAUUA CUCCAUCA | 3295 | UGAUGGAG CUGAUGAGGCCAGUUGGCCGAA AAUCUUGA | 12705 |
| 1876 | AGAUUACUC CAUCACUC | 3296 | GAGUGAUG CUGAUGAGGCCAGUUGGCCGAA AGUAAUCU | 12706 |
| 1880 | UACUCCAUC ACUCUGAA | 3297 | UUCAGAGU CUGAUGAGGCCAGUUGGCCGAA AUGGAGUA | 12707 |
| 1884 | CCAUCACUC UGAACCUU | 3298 | AAGGUUCA CUGAUGAGGCCAGUUGGCCGAA AGUGAUGG | 12708 |
| 1892 | CUGAACCUU GUCAUCAA | 3299 | UGGAUGAC CUGAUGAGGCCAGUUGGCCGAA AGGUUCAG | 12709 |
| 1895 | AACCUUGUC AUCAAGAA | 3300 | UUCUUGAU CUGAUGAGGCCAGUUGGCCGAA ACAAGGUU | 12710 |
| 1898 | CUUGUCAUC AAGAACGU | 3301 | ACGUUCUU CUGAUGAGGCCAGUUGGCCGAA AUGACAAG | 12711 |
| 1909 | GAACGUGUC UCUAGAAG | 3302 | CUUCUAGA CUGAUGAGGCCAGUUGGCCGAA ACACGUUC | 12712 |
| 1911 | ACGUGUCUC UAGAAGAC | 3303 | GUCUUCUA CUGAUGAGGCCAGUUGGCCGAA AGACACGU | 12713 |
| 1913 | GUGUCUCUA GAAGACUC | 3304 | GAGUCUUC CUGAUGAGGCCAGUUGGCCGAA AGAGACAC | 12714 |
| 1921 | AGAAGACUC GGGCACCU | 3305 | AGGUGCCC CUGAUGAGGCCAGUUGGCCGAA AGUCUUCU | 12715 |
| 1930 | GGGCACCUA UGCGUGCA | 3306 | UGCACGCA CUGAUGAGGCCAGUUGGCCGAA AGGUGCCC | 12716 |
| 1952 | AGGAACAUA UACACAGG | 3307 | CCUGUGUA CUGAUGAGGCCAGUUGGCCGAA AUGUUCCU | 12717 |
| 1954 | GAACAUAUA CACAGGGG | 3308 | CCCCUGUG CUGAUGAGGCCAGUUGGCCGAA AUAUGUUC | 12718 |
| 1970 | GAAGACAUC CUUCGGAA | 3309 | UUCCGAAG CUGAUGAGGCCAGUUGGCCGAA AUGUCUUC | 12719 |
| 1973 | GACAUCCUU CGGAAGAC | 3310 | GUCUUCCG CUGAUGAGGCCAGUUGGCCGAA AGGAUGUC | 12720 |
| 1974 | ACAUCCUUC GGAAGACA | 3311 | UGUCUUCC CUGAUGAGGCCAGUUGGCCGAA AAGGAUGU | 12721 |
| 1988 | ACAGAAGUU CUCGUUAG | 3312 | CUAACGAG CUGAUGAGGCCAGUUGGCCGAA ACUUCUGU | 12722 |
| 1989 | CAGAAGUUC UCGUUAGA | 3313 | UCUAACGA CUGAUGAGGCCAGUUGGCCGAA AACUGCUG | 12723 |
| 1991 | GAAGUUCUC GUGAGAGA | 3314 | UCUCUAAC CUGAUGAGGCCAGUUGGCCGAA AGAACUGC | 12724 |
| 1994 | GUUCUCGUU AGAGAGUC | 3315 | GAAUCUCU CUGAUGAGGCCAGUUGGCCGAA ACGAGAAC | 12725 |
| 1995 | UUCUCGUUA GAGAUUCG | 3316 | CGAAUCUC CUGAUGAGGCCAGUUGGCCGAA AACGAGAA | 12726 |
| 2001 | UGAGAGAUG CGGAAGCG | 3317 | CGCUUCCG CUGAUGAGGCCAGUUGGCCGAA AUCUCUAA | 12727 |
| 2002 | UAGAGAUUC GGAAGCGC | 3318 | GCGCUUCC CUGAUGAGGCCAGUUGGCCGAA AAUCUCUA | 12728 |
| 2021 | CACCUGCUU CAAAACCU | 3319 | AGGUUUUG CUGAUGAGGCCAGUUGGCCGAA AGCAGGUG | 12729 |
| 2022 | ACCUGCUUC AAAACCUC | 3320 | GAGGUUUU CUGAUGAGGCCAGUUGGCCGAA AAGCAGGU | 12730 |
| 2030 | CAAAACCUC AGUGACUA | 3321 | UAGUCACU CUGAUGAGGCCAGUUGGCCGAA AGGUUUUG | 12731 |
| 2038 | CAGUGACUA CGAGGUCU | 3322 | AGACCUCG CUGAUGAGGCCAGUUGGCCGAA AGUCACUG | 12732 |
| 2045 | UACGAGGUC UCCAUCAG | 3323 | CUGAUGGA CUGAUGAGGCCAGUUGGCCGAA ACCUCGUA | 12733 |
| 2047 | CGAGGUCUC CAUCAGUG | 3324 | CACUGAUG CUGAUGAGGCCAGUUGGCCGAA AGACCUCG | 12734 |
| 2051 | GUCUCCAUC AGUGGCUC | 3325 | GAGCCACU CUGAUGAGGCCAGUUGGCCGAA AUGGAGAC | 12735 |
| 2059 | CAGUGGCUC UACGACCU | 3326 | AGGUCGUA CUGAUGAGGCCAGUUGGCCGAA AGCCACUG | 12736 |
| 2061 | GUGGCUCUA CGACCUUA | 3327 | UAAGGUCG CUGAUGAGGCCAGUUGGCCGAA AGAGCCAC | 12737 |
| 2068 | UACGACCUU AGACUGUC | 3328 | GACAGUCU CUGAUGAGGCCAGUUGGCCGAA AGGUCGUA | 12738 |
| 2069 | ACGACCUUA GACUGUCA | 3329 | UGACAGUC CUGAUGAGGCCAGUUGGCCGAA AAGGUCGU | 12739 |
| 2076 | UAGACUGUC AAGCUAGA | 3330 | UCUAGCUU CUGAUGAGGCCAGUUGGCCGAA ACAGUCUA | 12740 |
| 2082 | GUCAAGCUA GAGGUGUC | 3331 | GACACCUC CUGAUGAGGCCAGUUGGCCGAA AGCUUGAC | 12741 |
| 2090 | AGAGGUGUC CCCGCGCC | 3332 | GGCGCGGG CUGAUGAGGCCAGUUGGCCGAA ACACCUCU | 12742 |
| 2100 | CCGCGCCUC AGAUCACU | 3333 | AGUGAUCU CUGAUGAGGCCAGUUGGCCGAA AGGCGCGG | 12743 |
| 2105 | CCUCAGAUC ACUUGGUU | 378 | AACCAAGU CUGAUGAGGCCAGUUGGCCGAA AUCUGAGG | 9797 |
| 2109 | AGAUCACUU GGUUCAAA | 3334 | UUUGAACC CUGAUGAGGCCAGUUGGCCGAA AGUGAUCU | 12744 |
| 2113 | CACUUGGUU CAAAAACA | 3335 | UGUUGUUG CUGAUGAGGCCAGUUGGCCGAA ACCAAGUG | 12745 |
| 2114 | ACUUGGUUC AAAAACAA | 3336 | UUGUUUUU CUGAUGAGGCCAGUUGGCCGAA AACCAAGU | 9800 |
| 2132 | CACAAAAUA CAACAAGA | 383 | UCUUGUUG CUGAUGAGGCCAGUUGGCCGAA AUUUUGUG | 9802 |
| 2150 | CCGGGAAUU AUUUUAGG | 3337 | CCUAAAAU CUGAUGAGGCCAGUUGGCCGAA AUUCCCGG | 12746 |
| 2151 | CGGGAAUUA UUUUAGGA | 3338 | UCCUAAAA CUGAUGAGGCCAGUUGGCCGAA AAUUCCCG | 12747 |
| 2153 | GGAAUUAUU UUAGGACC | 386 | GGUCCUAA CUGAUGAGGCCAGUUGGCCGAA AUAAUUCC | 9805 |
| 2154 | GAAUUAUUU UAGGACCA | 387 | UGGUCCUA CUGAUGAGGCCAGUUGGCCGAA AAUAAUUC | 9806 |
| 2155 | AAUUAUUUU AGGACCAG | 388 | CUGGUCCU CUGAUGAGGCCAGUUGGCCGAA AAAUAAUU | 9807 |
| 2156 | AUUAUUUUA GGACCAGG | 389 | CCUGGUCC CUGAUGAGGCCAGUUGGCCGAA AAAAUAAU | 9808 |
| 2179 | CACGCUGUU UAUUGAAA | 390 | UUUCAAUA CUGAUGAGGCCAGUUGGCCGAA ACAGCGUG | 9809 |
| 2180 | ACGCUGUUU AUUGAAAG | 391 | CUUUCAAU CUGAUGAGGCCAGUUGGCCGAA AACAGCGU | 9810 |
| 2181 | CGCUGUUUA UUGAAAGA | 392 | UCUUUCAA CUGAUGAGGCCAGUUGGCCGAA AAACAGCG | 9811 |
| 2183 | CUGUUUAUU GAAAGAGU | 393 | ACUCUUUC CUGAUGAGGCCAGUUGGCCGAA AUAAACAG | 9812 |
| 2192 | GAAAGAGUC ACAGAGGA | 3339 | UCCUCUGU CUGAUGAGGCCAGUUGGCCGAA ACUCUUUC | 12748 |
| 2213 | GAGGGUGUC UAUAGGUG | 3340 | CACCUAUA CUGAUGAGGCCAGUUGGCCGAA ACACCCUC | 12749 |
| 2215 | GGGUGUCUA UAGGUGCC | 3341 | GGCACCUA CUGAUGAGGCCAGUUGGCCGAA AGACACCC | 12750 |
| 2217 | GUGUCUAUA GGUGCCGA | 3342 | UCGGCACC CUGAUGAGGCCAGUUGGCCGAA AUAGACAC | 12751 |
| 2263 | CGCAGCCUA CCUCACCG | 3343 | CGGUGAGG CUGAUGAGGCCAGUUGGCCGAA AGGCUGCG | 12752 |
| 2267 | GCCUACCUC ACCGUGCA | 3344 | UGCACGGU CUGAUGAGGCCAGUUGGCCGAA AGGUAGGC | 12753 |
| 2284 | AGGAACCUC AGACAAGU | 3345 | ACUUGUCU CUGAUGAGGCCAGUUGGCCGAA AGGUUCCU | 12754 |
| 2293 | AGACAAGUC AAACCUGG | 3346 | CCAGGUUU CUGAUGAGGCCAGUUGGCCGAA ACUUGUCU | 12755 |
| 2309 | GACCUGAUC ACGCUCAC | 3347 | GUGAGCGU CUGAUGAGGCCAGUUGGCCGAA AUCAGCUC | 12756 |
| 2315 | AUCACGCUC ACGUGCAC | 3348 | GUGCACGU CUGAUGAGGCCAGUUGGCCGAA AGCGUGAU | 12757 |
| 2342 | GCGACCCUC UUUUGGCU | 3349 | AGCCAAAA CUGAUGAGGCCAGUUGGCCGAA AGGGUCGC | 12758 |
| 2344 | GACCCUCUU UUGGCUCC | 3350 | GGAGCCAA CUGAUGAGGCCAGUUGGCCGAA AGAGGGUC | 12759 |
| 2345 | ACCCUCUUU UGGCUCCU | 3351 | AGGAGCCA CUGAUGAGGCCAGUUGGCCGAA AAGAGGGU | 12760 |
| 2346 | CCCUCUUUU GGCUCCUU | 3352 | AAGGAGCC CUGAUGAGGCCAGUUGGCCGAA AAAGAGGG | 12761 |
| 2351 | UUUUGGCUC CUUCUAAC | 3353 | GUUAGAAG CUGAUGAGGCCAGUUGGCCGAA AGCCAAAA | 12762 |
| 2354 | UGGCUCCUU CUAACUCU | 3354 | AGAGUUAG CUGAUGAGGCCAGUUGGCCGAA AGGAGCCA | 12763 |
| 2355 | GGCUCCUUC UAACUCUC | 3355 | GAGAGUUA CUGAUGAGGCCAGUUGGCCGAA AAGGAGCC | 12764 |
| 2357 | CUCCUUCUA ACUCUCUU | 3356 | AAGAGAGU CUGAUGAGGCCAGUUGGCCGAA AGAAGGAG | 12765 |
| 2361 | UUCUAACUC UCUUCAUC | 3357 | GAUGAAGA CUGAUGAGGCCAGUUGGCCGAA AGUUAGAA | 12766 |
| 2363 | CUAACUCUC UUCAUCAG | 3358 | CUGAUGAA CUGAUGAGGCCAGUUGGCCGAA AGAGUUAG | 12767 |
| 2365 | AACUCUCUU CAUCAGAA | 3359 | UUCUGAUG CUGAUGAGGCCAGUUGGCCGAA AGAGAGUU | 12768 |
| 2366 | ACUCUCUUC AUCAGAAA | 3360 | UUUCUGAU CUGAUGAGGCCAGUUGGCCGAA AAGAGAGU | 12769 |
| 2369 | CUCUUCAUC AGAAAACU | 3361 | AGUUUUCU CUGAUGAGGCCAGUUGGCCGAA AUGAAGAG | 12770 |
| 2386 | GAAGCGGUC UUCUUCCG | 3362 | CGGAAGAA CUGAUGAGGCCAGUUGGCCGAA ACCGCUUC | 12771 |
| 2388 | AGCGGUCUU CUUCCGAA | 3363 | UUCGGAAG CUGAUGAGGCCAGUUGGCCGAA AGACCGCU | 12772 |
| 2389 | GCGGUCUUC UUCCGAAG | 3364 | CUUCGGAA CUGAUGAGGCCAGUUGGCCGAA AAGACCGC | 12773 |
| 2391 | GGUCUUCUU CCGAAGUA | 3365 | UACUUCGG CUGAUGAGGCCAGUUGGCCGAA AGAAGACC | 12774 |
| 2392 | GUCUUCUUC CGAAGUAA | 3366 | UUACUUCG CUGAUGAGGCCAGUUGGCCGAA AAGAAGAC | 12775 |
| 2399 | UCCGAAGUA AAGACAGA | 3367 | UCUGUCUU CUGAUGAGGCCAGUUGGCCGAA ACUUCGGA | 12776 |
| 2410 | GACAGACUA CCUGUCAA | 3368 | UUGACAGG CUGAUGAGGCCAGUUGGCCGAA AGUCUGUC | 12777 |
| 2416 | CUACCUGUC AAUCAUUA | 3369 | UAAUGAUU CUGAUGAGGCCAGUUGGCCGAA ACAGGUAG | 12778 |
| 2420 | CUGUCAAUC AUUAUGGA | 3370 | UCCAUAAU CUGAUGAGGCCAGUUGGCCGAA AUUGACAG | 12779 |
| 2423 | UCAAUCAUU AUGGACCC | 3371 | GGGUCCAU CUGAUGAGGCCAGUUGGCCGAA AUGAUUGA | 12780 |
| 2424 | CAAUCAUUA UGGACCCA | 3372 | UGGGUCCA CUGAUGAGGCCAGUUGGCCGAA AAUGAUUG | 12781 |
| 2441 | GAUGAAGUU CCCCUGGA | 3373 | UCCAGGGG CUGAUGACGCCGUUAGGCCGAA ACUUCAUC | 12782 |
| 2442 | AUGAAGUUC CCCUGGAU | 3374 | AUCCAGGO CUGAUGAGGCCGUUAGGCCGAA AACUUCAU | 12783 |
| 2473 | GCUGCCCUA UGAUGCCA | 3375 | UGGCAUCA CUGAUGAGGCCGUUAGGCCGAA AGGGCAGC | 12784 |
| 2494 | GUGGGAGUU UGCACGGG | 3376 | CCCGUGCA CUGAUGAGGCCGUUAGGCCGAA ACUCCCAC | 12785 |
| 2495 | UGGGAGUUU GCACCGGA | 3377 | UCCCGUGC CUGAUGAGGCCGUUAGGCCGAA AACUCCCA | 12786 |
| 2516 | CUGAAACUA GGCAAAUC | 3378 | GAUUUGCC CUGAUGAGGCCGUUAGGCCGAA AGUUUCAG | 12787 |
| 2524 | AGGCAAAUC GCUCGGAA | 3379 | UUCCGAGC CUGAUGAGGCCGUUAGGCCGAA AUUUGCCU | 12788 |
| 2528 | AAAUCGCUC GGAAGAGG | 3380 | CCUCUUCC CUGAUGAGGCCGUUAGGCCGAA AGCGAUUU | 12789 |
| 2541 | GAGGGGCUU UUGGGAAA | 3381 | UUUCCCAA CUGAUGAGGCCGUUAGGCCGAA AGCCCCUC | 12790 |
| 2542 | AGGGGCUUU UGGGAAAG | 3382 | CUUUCCCA CUGAUGAGGCCGUUAGGCCGAA AAGCCCCU | 12791 |
| 2543 | GGGGCUUUU GGGAAAGU | 3383 | ACUUUCCC CUGAUGAGGCCGUUAGGCCGAA AAAGCCCC | 12792 |
| 2552 | GGGAAAGUC GUUCAAGC | 3384 | GCUUGAAC CUCAUGAGGCCGUUAGGCCGAA ACUUUCCC | 12793 |
| 2555 | AAAGUCGUU CAAGCCUC | 3385 | GAGGCUUG CUGAUGAGGCCGUUAGGCCGAA ACGACUUU | 12794 |
| 2556 | AAGUCGUUC AAGCCUCU | 3386 | AGAGGCUU CUGAUGAGGCCGUUAGGCCGAA AACGACUU | 12795 |
| 2563 | UCAAGCCUC UGCAUUUG | 3387 | CAAAUGCA CUGAUGAGGCCGUUAGGCCGAA AGGCUUGA | 12796 |
| 2569 | CUCUGCAUU UGGCAUUA | 3388 | UAAUGCCA CUGAUGAGGCCGUUAGGCCGAA AUGCAGAG | 12797 |
| 2570 | UCUGCAUUU GGCAUUAA | 3389 | UUAAUGCC CUGAUGAGGCCGUUAGGCCGAA AAUGCAGA | 12798 |
| 2576 | UUUGGCAUU AAGAAAUC | 457 | GAUUUCUU CUGAUGAGGCCGUUAGGCCGAA AUGCCAAA | 9876 |
| 2577 | UUGGCAUUA AGAAAUCA | 458 | UGAUUUCU CUGAUGAGGCCGUUAGGCCGAA AAUGCCAA | 9877 |
| 2584 | UAAGAAAUC ACCCACCU | 3390 | AGGUGGGU CUGAUCAGGCCGUUAGGCCGAA AUUUCUUA | 12799 |
| 2617 | GAAGAUGUU GAAAGAGG | 3391 | CCUCUUUC CUGAUGAGGCCGUUAGGCCGAA ACAUCUUC | 12800 |
| 2644 | CAGUGAGUA CAAAGCUC | 3392 | GAGCUUUG CUGAUGAGGCCGUUAGGCCGAA ACUCACUG | 12801 |
| 2652 | ACAAAGCUC UGAUCACC | 3393 | GGUCAUCA CUGAUGAGGCCGUUAGGCCGAA AGCUUUGU | 12802 |
| 2666 | ACCGAACUC AA3AUCUU | 3394 | AAGAUCUU CUGAUGAGGCCGUUAGGCCGAA AGUUCGGU | 12803 |
| 2672 | CUCAAGAUC UUGACCCA | 3395 | UGGGUCAA CUGAUGAGGCCGUUAGGCCGAA AUCUUGAG | 12804 |
| 2674 | CAAGAUCUU GACCCACA | 3396 | UGUGGGUC CUGAUGAGGCCGUUAGGCCGAA AGAUCUUC | 12805 |
| 2684 | ACCCACAUC GGCCAUCA | 3397 | UGAUGGCC CUGAUGAGGCCGUUAGGCCGAA AUGUGGGU | 12806 |
| 2691 | UCGCCCAUC AUCUGAAU | 3398 | AUUCAGAU CUGAUGAGGCCGUUAGGCCGAA AUGGCCGA | 12807 |
| 2694 | GCCAUCAUC UGAAUGUG | 3399 | CACAUUCA CUGAUGAGGCCGUUAGGCCGAA AUGAUGOC | 12808 |
| 2705 | AAUGUGGUU AACCUCCU | 3400 | AGGAGGUU CUGAUGAGGCCGUUAGGCCGAA ACCACAUU | 12809 |
| 2706 | AUGUGGUUA ACCUCCUG | 3401 | CAGGAGGU CUGAUGAGGCCGUUAGGCCGAA AACCACAU | 12810 |
| 2711 | GUUAACCUC CUGGGAGC | 3402 | GCUCCCAG CUGAUGAGGCCGUUAGGCCGAA AGGUUAAC | 12811 |
| 2742 | GAGGGCCUC UGAUGGUG | 470 | CACCAUCA CUGAUGAGGCCGUUAGGCCGAA AGGCCCUC | 9889 |
| 2753 | AUGGUGAUC GUGGAAUA | 3403 | UAUUCCAC CUGAUGAGGCCGUUAGGCCGAA AUCACCAU | 12812 |
| 2761 | CGUGGAAUA CUGCAAAU | 3404 | AUUUGCAG CUGAUGAGGCCGUUAGGCCGAA AGUCCACO | 12813 |
| 2770 | CUGCAAAUA CGGAAACC | 3405 | GGUUUCCG CUGAUGAGGCCGUUAGGCCGAA AUUUGCAG | 12814 |
| 2782 | AAACCUGUC CAACUACC | 3406 | GGUAGUUG CUGAUGAGGCCGUUAGGCCGAA ACAGGUUU | 12815 |
| 2788 | GUCCAACUA CCUCAAGA | 3407 | UCUUGAGG CUGAUGAGGCCGUUAGGCCGAA AGUUGGAC | 12816 |
| 2792 | AACUACCUC AAGAGCAA | 479 | UUGCUCUU CUGAUGAGGCCGUUAGGCCGAA AGGUAGUU | 9898 |
| 2809 | ACGUGACUU AUUCUGUC | 3408 | GACAGAAU CUGAUGAGGCCGUUAGGCCGAA AGUCACGU | 12817 |
| 2810 | CGUGACUUA UUCUGUCU | 3409 | AGACAGAA CUGAUGAGGCCGUUAGGCCGAA AAGUCACG | 12818 |
| 2812 | UGACUUAUU CUGUCUCA | 3410 | UGAGACAG CUGAUGAGGCCGUUAGGCCGAA AUAAGUCA | 12819 |
| 2813 | GACUUAUUC UGUCUCAA | 3411 | UUGAGACA CUGAUGAGGCCGUUAGGCCGAA AAUAAGUC | 12820 |
| 2817 | UAUUCUGUC UCAACAAG | 3412 | CUUGUUGA CUGAUGAGGCCGUUAGGCCGAA ACAGAAUA | 12821 |
| 2819 | UUCUGUCUC AACAAGGA | 3413 | UCCUUGUU CUGAUGAGGCCGUUAGGCCGAA AGACAGAA | 12822 |
| 2836 | CGCAGCCUU GCAUAUGG | 3414 | CCAUAUGC CUGAUGAGGCCGUUAGGCCGAA AGGCUGCG | 12823 |
| 2841 | CCUUGCAUA UGGAGCUC | 3415 | GAGCUCCA CUGAUGAGGCCGUUAGGCCGAA AUGCAAGG | 12824 |
| 2849 | AUGGAGCUC AAGAAAGA | 3416 | UCUUUCUU CUGAUGAGGCCGUUAGGCCGAA AGCUCCAU | 12825 |
| 2900 | CCCCGCCUA GACAGUGU | 3417 | ACACUGUC CUGAUGAGGCCGUUAGGCCGAA AGGCGGGG | 12826 |
| 2909 | GACAGUGUC AGCAGCUC | 3418 | GAGCUGCU CUGAUGAGGCCGUUAGGCCGAA ACACUGUC | 12827 |
| 2917 | CAGCAGCUC AAGUGUCA | 3419 | UGACACUU CUGAUGAGGCCGUUAGGCCGAA AGCUGCUG | 12828 |
| 2924 | UCAAGUGUC ACCAGCUC | 3420 | GAGCUGGU CUGAUGAGGCCGUUAGGCCGAA ACACUUGA | 12829 |
| 2932 | CACCAGCUC CAGCUUCC | 3421 | GGAAGCUG CUGAUGAGGCCGUUAGGCCGAA ACCUGGUG | 12830 |
| 2938 | CUCCAGCUU CCCUGAAG | 3422 | CUUCAGGG CUGAUGAGGCCGUUAGGCCGAA AGCUGGAG | 12831 |
| 2939 | UCCAGCUUC CCUGAAGA | 3423 | UCUUCAGG CUGAUGAGGCCGUUAGGCCGAA AAGCUGGA | 12832 |
| 2982 | ACGAGGAUU ACAGUGAG | 3424 | CUCACUGU CUGAUGAGGCCGUUAGGCCGAA AUCCUCGU | 12833 |
| 2983 | CGAGGAUUA CAGUGAGA | 3425 | UCUCACUG CUGAUGAGGCCGUUAGGCCGAA AAUCCUCG | 12834 |
| 2993 | AGUGAGAUC UCCAAGCA | 3426 | UGCUUGGA CUGAUGAGGCCGUUAGGCCGAA AUCUCACU | 12835 |
| 2995 | UGAGAUCUC CAAGCAGC | 3427 | GCUGCUUG CUGAUGAGGCCGUUAGGCCGAA AGAUCUCA | 12836 |
| 3008 | CAGCCCCUC ACCAUGGA | 3428 | UCCAUGGU CUGAUGAGGCCGUUAGGCCGAA AGGGGCUG | 12837 |
| 3026 | GACCUGAUU UCCUACAG | 3429 | CUGUAGGA CUGAUGAGGCCGUUAGGCCGAA AUCAGGUC | 12838 |
| 3027 | ACCUGAUUU CCUACAGU | 3430 | ACUGUAGG CUGAUGAGGCCGUUAGGCCGAA AAUCAGGU | 12839 |
| 3028 | CCUGAUUUC CUACAGUU | 3431 | AACUGUAG CUGAUGAGGCCGUUAGGCCGAA AAAUCAGG | 12840 |
| 3031 | GAUUUCCUA CAGUUUCC | 3432 | GGAAACUG CUGAUGAGGCCGUUAGGCCGAA AGGAAAUC | 12841 |
| 3036 | CCUACAGUU UCCAAGUG | 3433 | CACUUGGA CUGAUGAGGCCGUUAGGCCGAA ACUGUAGG | 12842 |
| 3037 | CUACAGUUU CCAAGUGG | 3434 | CCACUUGG CUGAUGAGGCCGUUAGGCCGAA AACUGUAG | 12843 |
| 3038 | UACAGUUUC CAAGUGGC | 3435 | GCCACUUG CUGAUGAGGCCGUUAGGCCGAA AAACUGUA | 9938 |
| 3061 | CAUGGAGUU UCUGUCCU | 3436 | AGGACAGA CUGAUGAGGCCGUUAGGCCGAA ACUCCAUG | 12844 |
| 3062 | AUGGAGUUU CUGUCCUC | 3437 | GAGGACAG CUGAUGAGGCCGUUAGGCCGAA UACUCCAU | 12845 |
| 3063 | UGGAGUUUC UGUCCUCC | 3438 | GGAGGACA CUGAUGAGGCCGUUAGGCCGAA AAACUCCA | 12846 |
| 3067 | GUUUCUGUC CUCCAGAA | 3439 | UUCUGGAG CUGAUGAGGCCGUUAGGCCGAA ACAGAAAC | 12847 |
| 3070 | UCUGUCCUC CAGAAAGU | 3440 | ACUUUCUG CUGAUGAGGCCGUUAGGCCGAA AGGACAGA | 12848 |
| 3083 | AAGUGCAUU CAUCGGGA | 526 | UCCCGAUG CUGAUGAGGCCGUUAGGCCGAA AUGCACUU | 9945 |
| 3084 | AGUGCAUUC AUCGGGAC | 527 | GUCCCGAU CUGAUGAGGCCGUUAGGCCGAA AAUGCACU | 9946 |
| 3087 | GCAUUCAUC GGGACCUG | 528 | CAGGUCCC CUGAUGAGGCCGUUAGGCCGAA AUGAAUGC | 9947 |
| 3110 | AGAAACAUC CUUUUAUC | 3441 | GAUAAAAG CUGAUGAGGCCGUUAGGCCGAA AUGUUUCU | 9948 |
| 3113 | AACAUCCUU UUAUCUGA | 3442 | UCAGAUAA CUGAUGAGGCCGUUAGGCCGAA AGGAUGUU | 12849 |
| 3114 | ACAUCCUUU UAUCUGAG | 3443 | CUCAGAUA CUGAUGAGGCCGUUAGGCCGAA AAGGAUGU | 12850 |
| 3115 | CAUCCUUUU AUCUGAGA | 3444 | UCUCAGAU CUGAUGAGGCCGUUAGGCCGAA AAAGGAUG | 12851 |
| 3116 | AUCCUUUUA UCUGAGAA | 3445 | UUCUCAGA CUGAUGAGGCCGUUAGGCCGAA AAAAGGAU | 12852 |
| 3118 | CCUUUUAUC UGAGAACA | 3446 | UGUUCUCA CUGAUGAGGCCGUUAGGCCGAA AUAAAAGG | 12853 |
| 3140 | GUGAAGAUU UGCGACUU | 3447 | AAGUCGCA CUGAUGAGGCCGUUAGGCCGAA AUCUUCAC | 12854 |
| 3141 | UGAAGAUUU GCGACUUU | 3448 | AAAGUCGC CUGAUGAGGCCGUUAGGCCGAA AAUCUUCA | 12855 |
| 3148 | UUGCGACUU UGCCCUGG | 3449 | CCAGGCCA CUGAUGAGGCCGUUAGGCCGAA AGUCGCAA | 12856 |
| 3149 | UGCGACUUU GGCCUGGC | 3450 | GCCAGGCC CUGAUGAGGCCGUUAGGCCGAA AAGUCGCA | 12857 |
| 3165 | CCCGGGAUA UUUAUAAG | 542 | CUUAUAAA CUGAUGAGGCCGUUAGGCCGAA AUCCCGGG | 9961 |
| 3167 | CGGGAUAUU UAUAAGAA | 543 | UUCUUAUA CUGAUGAGGCCGUUAGGCCGAA AUAUCCCG | 9962 |
| 3168 | GGGAUAUUU AUAAGAAC | 544 | GUUCUUAU CUGAUGAGGCCGUUAGGCCGAA AAUAUCCC | 9963 |
| 3169 | GGAUAUUUA UAAGAACC | 545 | GGUUCUUA CUGAUGAGGCCGUUAGGCCGAA AAAUAUCC | 9964 |
| 3171 | AUAUUUAUA AGAACCCU | 3451 | AGGGUUCU CUGAUGAGGCCGUUAGGCCGAA AUAAAUAU | 12858 |
| 3183 | ACCCUGAUU AUGUGAGG | 3452 | CCUCACAU CUGAUGAGGCCGUUAGGCCGAA AUCAGGGU | 12859 |
| 3184 | CCCUGAUUA UGUCAGGA | 3453 | UCCUCACA CUGAUGAGGCCGUUAGGCCGAA AAUCAGGG | 12860 |
| 3201 | GAGGAGAUA CUCGACUU | 3454 | AAGUCGAG CUGAUGAGGCCGUUAGGCCGAA AUCUCCUC | 12861 |
| 3204 | GAGAUACUC GACUUCCC | 3455 | GGGAAGUC CUGAUGAGGCCGUUAGGCCGAA AGUAUCUC | 12862 |
| 3209 | ACUCGACUU CCCCUAAA | 3456 | UUUAGGGG CUGAUGAGGCCGUUAGGCCGAA AGUCGAGU | 12863 |
| 3210 | CUCGACUUC CCCUAAAA | 3457 | UUUUAGGG CUGAUGAGGCCGUUAGGCCGAA AAGUCGAG | 12864 |
| 3215 | CUUCCCCUA AAAUGGAU | 3458 | AUCCAUUU CUGAUGAGGCCGUUAGGCCGAA AGGGGAAG | 12865 |
| 3228 | GGAUGGCUC CUGAAUCC | 3459 | GGAUUCAG CUGAUGAGGCCGUUAGGCCGAA AGCCAUCC | 12866 |
| 3235 | UCCUGAAUC CAUCUUUG | 3460 | CAAAGAUG CUGAUGAGGCCGUUAGGCCGAA AUUCAGGA | 12867 |
| 3239 | GAAUCCAUC UUUGACAA | 3461 | UUGUCAAA CUGAUGAGGCCGUUAGGCCGAA AUGGAUUC | 12868 |
| 3241 | AUCCAUCUU UGACAAGG | 3462 | CCUUGUCA CUGAUGAGGCCGUUAGGCCGAA AGAUGGAU | 12869 |
| 3242 | UCCAUCUUU GACAAGGU | 3463 | ACCUUGUC CUGAUGAGGCCGUUAGGCCGAA AAGAUGGA | 12870 |
| 3251 | GACAACGUC UACAGCAC | 3464 | GUGCUGUA CUGAUGAGGCCGUUAGGCCGAA ACCUUGUC | 12871 |
| 3253 | CAAGGUCUA CAGCACCA | 3465 | UGGUOCUG CUGAUGAGGCCGUUAGGCCGAA AGACCUUG | 12872 |
| 3277 | UGUGUGGUC CUAUGGCG | 3466 | CGCCAUAG CUGAUGAGGCCGUUAGGCCGAA ACCACACA | 12873 |
| 3280 | GUGGUCCUA UGGCGUGU | 3467 | ACACGCCA CUGAUGAGGCCGUUAGGCCGAA AGGACCAC | 12874 |
| 3289 | UGGCGUGUU GCUGUGGG | 3468 | CCCACAGC CUGAUGAGGCCGUUAGGCCGAA ACACGCCA | 12875 |
| 3302 | UGGGAGAUC UUCUCCUU | 3469 | AAGGAGAA CUGAUGAGGCCGUUAGGCCGAA AUCUCCCA | 12876 |
| 3304 | GGAGAUCUU CUCCUUAG | 3470 | CUAAGGAG CUGAUGAGGCCGUUAGGCCGAA AGAUCUCC | 12877 |
| 3305 | GAGAUCUUC UCCUUAGG | 3471 | CCUAAGGA CUGAUGAGGCCGUUAGGCCGAA AAGAUCUC | 12878 |
| 3307 | GAUCUUCUC CUUAGGGG | 3472 | CCCCUAAG CUGAUGAGGCCGUUAGGCCGAA AGAAGAUC | 12879 |
| 3310 | CUUCUCCUU AGGGGGUU | 3473 | AACCCCCU CUGAUGAGGCCGUUAGGCCGAA AGGAGAAG | 12880 |
| 3311 | UUCUCCUUA GGGGGUUC | 3474 | GAACCCCC CUGAUGAGGCCGUUAGGCCGAA AAGGAGAA | 12881 |
| 3318 | UAGGGGGUU CUCCAUAC | 3475 | GUAUGGAG CUGAUGAGGCCGUUAGGCCGAA ACCCCCUA | 12882 |
| 3319 | AGGGGGUUC UCCAUACC | 3476 | GGUAUGGA CUGAUGAGGCCGUUAGGCCGAA AACCCCCU | 12883 |
| 3321 | GGGGUUCUC CAUACCCA | 3477 | UGGGUAUG CUGAUGAGGCCGUUAGGCCGAA AGAACCCC | 12884 |
| 3325 | UUCUCCAUA CCCAGGAG | 3478 | CUCCUGGG CUGAUGAGGCCGUUAGGCCGAA AUGGAGAA | 12885 |
| 3352 | UGAAGACUU CUGCAGCC | 3479 | GGCUGCAG CUGAUGAGGCCGUUAGGCCGAA AGUCUUCA | 12886 |
| 3353 | GAAGACUUC UGCAGCCG | 3480 | CGGCUGCA CUGAUGAGGCCGUUAGGCCGAA AAGUCUUC | 12887 |
| 3397 | CCCGGAGUA UGCCACAC | 3481 | GUGUGGCA CUGAUGAGGCCGUUAGGCCGAA ACUCCGGG | 12888 |
| 3413 | CCUGAAAUC UACCAAAU | 3482 | AUUUGGUA CUGAUGAGGCCGUUAGGCCGAA AUUUCAGG | 12889 |
| 3415 | UGAAAUCUA CCAAAUCA | 3483 | UGAUUUGG CUGAUGAGGCCGUUAGGCCGAA AGAUUUCA | 12890 |
| 3422 | UACCAAAUC AUGUUGGA | 3484 | UCCAACAU CUGAUGAGGCCGUUAGGCCGAA AUUUGGUA | 12891 |
| 3427 | AAUCAUGUU GGAUUGCU | 3485 | AGCAAUCC CUGAUGAGGCCGUUAGGCCGAA ACAUGAUU | 12892 |
| 3432 | UGUUGGAUU GCUGGCAC | 3486 | GUGCCAGC CUGAUGAGGCCGUUAGGCCGAA AUCCAACA | 12893 |
| 3466 | GCCCCGGUU UGCUGAAC | 3487 | GUUCAGCA CUGAUGAGGCCGUUAGGCCGAA ACCOGGOC | 12894 |
| 3467 | CCCCGGUUU GCUGAACU | 3488 | AGUUCAGC CUGAUGAGGCCGUUAGGCCGAA AACCGGGG | 12895 |
| 3476 | GCUGAACUU GUGGAGAA | 3489 | UUCUCCAC CUGAUGAGGCCGUUAGGCCGAA AGUUCAGC | 12896 |
| 3488 | GAGAAACUU GGUGACCU | 3490 | AGGUCACC CUGAUGAGGCCGUUAGGCCGAA AGUUUCUC | 12897 |
| 3500 | GACCUGCUU CAAGCCAA | 3491 | UUGGCUUG CUGAUGAGGCCGUUAGGCCGAA ACCAGGUC | 12898 |
| 3501 | ACCUGCUUC AAGCCAAC | 3492 | GUUGGCUU CUGAUGAGGCCGUUAGGCCGAA AAGCAGGU | 12899 |
| 3512 | GCCAACGUC CAACAGGA | 3493 | UCCUGUUG CUGAUGAGGCCGUUAGGCCGAA ACGUUGGC | 12900 |
| 3531 | GGAAAGAUU ACAUCCCC | 3494 | GGGGAUGU CUGAUGAGGCCGUUAGGCCGAA AUCUUUCC | 12901 |
| 3532 | GAAAGAUUA CAUCCCCC | 3495 | GGGGGAUG CUGAUGAGGCCGUUAGGCCGAA AAUCUUUC | 12902 |
| 3536 | GAUUACAUC CCCCUCAA | 3496 | UUGAGGGG CUGAUGAGGCCGUUAGGCCGAA AUGUAAUC | 12903 |
| 3542 | AUCCCCCUC AAUGCCAU | 3497 | AUGGCAUU CUGAUGAGGCCGUUAGGCCGAA AGGGGGAU | 12904 |
| 3551 | AAUGCCAUA CUGACUAG | 3498 | CUAGUCAG CUGAUGAGGCCGUUAGGCCGAA AUGGCAUU | 12905 |
| 3558 | UACUGACUA GAAACAGU | 3499 | ACUGUHUC CUGAUGAGGCCGUUAGGCCGAA AGUCAGUA | 12906 |
| 3567 | GAAACAGUA GCUUCACA | 3500 | UGUGAAGC CUGAUGAGGCCGUUAGGCCGAA ACUGUUUC | 12907 |
| 3571 | CAGUAGCUU CACAUACU | 3501 | AGUAUGUG CUGAUGAGGCCGUUAGGCCGAA ACCUACUG | 12908 |
| 3572 | AGUAGCUUC ACAUACUC | 3502 | GAGUAUGU CUGAUGAGGCCGUUAGGCCGAA AAGCUACU | 12909 |
| 3577 | CUUCACAUA CUCGACCC | 3503 | GGGUCCAG CUGAUGAGGCCGUUAGGCCGAA AUGUGAAG | 12910 |
| 3580 | CACAUACUC GACCCCCA | 3504 | UGGGGGUC CUGAUGAGGCCGUUAGGCCGAA AGUAUGUG | 12911 |
| 3592 | CCCCACCUU CUCUGAGG | 3505 | CCUCAGAG CUGAUGAGGCCGUUAGGCCGAA AGGUGGUG | 12912 |
| 3593 | CCCACCUUC UCUGAGGA | 3506 | UCCUCAGA CUGAUGAGGCCGUUAGGCCGAA AAGGUGGG | 12913 |
| 3595 | CACCUUCUC UGAGGACC | 3507 | GGUCCUCA CUGAUGAGGCCGUUAGGCCGAA AGAAGGUG | 12914 |
| 3605 | GAGGACCUU UUCAAGGA | 3508 | UCCUUGAA CUGAUGAGGCCGUUAGGCCGAA AGGUCCUC | 12915 |
| 3606 | AGGACCUUU UCAAGGAC | 3509 | GUCCUUGA CUGAUGAGGCCGUUAGGCCGAA AAGGUCCU | 12916 |
| 3607 | GGACCUUUU CAAGGACG | 3510 | CGUCCUUG CUGAUGAGGCCGUUAGGCCGAA AAAGGUCC | 12917 |
| 3608 | GACCUUUUC AAGGACGG | 3511 | CCGUCCUU CUGAUGAGGCCGUUAGGCCGAA AAAAGGUC | 12918 |
| 3619 | CGACGGCUU UGCAGAUC | 3512 | GAUCUGCA CUGAUGAGGCCGUUAGGCCCAA AGCCGUCC | 12919 |
| 3620 | GACGGCUUU GCAGAUCC | 3513 | GGAUCUGC CUGAUGAGGCCGUUAGGCCGAA AAGCCGUC | 12920 |
| 3627 | UUGCAGAUC CACAUUUU | 3514 | AAAAUGUG CUGAUGAGGCCGUUAGGCCGAA AUCUGCAA | 12921 |
| 3633 | AUCCACAUU UUCAUUCC | 3515 | GGAAUGAA CUGAUGAGGCCGUUAGGCCGAA AUGUGGAU | 12922 |
| 3634 | UCCACAUUU UCAUUCCG | 3516 | CGGAAUGA CUGAUGAGGCCGUUAGGCCGAA AAUGUGGA | 12923 |
| 3635 | CCACAUUUU CAUUCCGG | 3517 | CCGGAAUG CUGAUGAGGCCGUUAGGCCGAA AAAUGUGG | 12924 |
| 3636 | CACAGUOUC AUUCCGGA | 3518 | UCCGGAAU CUGAUGAGGCCGUUAGGCCGAA AAAAUGUG | 12925 |
| 3639 | AUUUUCAUU CCGGAAGC | 3519 | GCUUCCGG CUGAUGAGGCCGUUAGGCCGAA AUGAAAAU | 12926 |
| 3640 | UUUUCAUUC CGGAAGCU | 3520 | AGCUUCCG CUGAUGAGGCCGUUAGGCCGAA AAUGAAAA | 12927 |
| 3649 | CGGAAGCUC UGAUGAUG | 3521 | CAUCAUCA CUGAUGAGGCCGUUAGGCCGAA AGCUUCCG | 12928 |
| 3664 | UGUGAGAUA UGUAAACG | 3522 | CGUUUACA CUGAUGAGGCCGUUAGGCCGAA AUCUCACA | 12929 |
| 3668 | AGAUAUGUA AACGCUUU | 3523 | AAAGCGUU CUGAUGAGGCCGUUAGGCCGAA ACAUAUCU | 12930 |
| 3675 | UAAACGCUU UCAAAUUC | 3524 | GAAUUUGA CUGAUGAGGCCGUUAGGCCGAA AGCGUUUA | 12931 |
| 3676 | AAACGCUUU CAAAUUCA | 3525 | UGAAUUUG CUGAUGAGGCCGUUAGGCCGAA AAGCGUUU | 12932 |
| 3677 | AACGCUUUC AAAUUCAU | 3526 | AUGAAUUU CUGAUGAGGCCGUUAGGCCGAA AAAGCGUU | 12933 |
| 3682 | UUUCAAAUU CAUGAGCC | 3527 | GGCUCAUG CUGAUGAGGCCGUUAGGCCGAA AUUUGAAA | 12934 |
| 3683 | UUCAAAUUC AUGAGCCU | 3528 | AGGCUCAU CUGAUGAGGCCGUUAGGCCGAA AAUUUGAA | 12935 |
| 3701 | GAAAGAAUC AAAACCUU | 637 | AAGGUUUU CUGAUGAGGCCGUUAGGCCGAA AUUCUUUC | 10056 |
| 3709 | CAAAACCUU UGAGGAUC | 3529 | GCUCCUCA CUGAUGAGGCCGUUAGGCCGAA AGGUUUUG | 12936 |
| 3710 | AAAACCUUU GAGGAGCU | 3530 | AGCUCCUC CUGAUGAGGCCGUUAGGCCGAA AAGGUUUU | 12937 |
| 3719 | GAGGAGCUU UCACCGAA | 3531 | UUCGGUGA CUGAUGAGGCCGUUAGGCCGAA AGCUCCUC | 12938 |
| 3720 | AGGAGCUUU CACCGAAC | 3532 | GUUCGGUG CUGAUGAGGCCGUUAGGCCGAA AAGCUCCU | 12939 |
| 3721 | GGAGCUUUC ACCGAACU | 3533 | AGUUCGGU CUGAUGAGGCCGUUAGGCCGAA AAAGCUCC | 12940 |
| 3730 | ACCGAACUC CACCUCCA | 3534 | UGGAGGUG CUGAUGAGGCCGUUAGGCCGAA AGUUCGGU | 12941 |
| 3736 | CUCCACCUC CAUGUUUG | 3535 | CAAACAUG CUGAUGAGGCCGUUAGGCCGAA AGGUGGAG | 12942 |
| 3742 | CUCCAUGUU UGAGGACU | 3536 | AGUCCUCA CUGAUGAGGCCGUUAGGCCGAA ACAUGGAG | 12943 |
| 3743 | UCCAUGUUU GAGGACUA | 3537 | UAGUCCUC CUGAUGAGGCCGUUAGGCCGAA AACAUGGA | 12944 |
| 3751 | UGAGGACUA UCAGCUGG | 3538 | CCAGCUGA CUGAUGAGGCCGUUAGGCCGAA AGUCCUCA | 12945 |
| 3753 | AGGACUAUC AGCUGGAC | 3539 | GUCCAGCU CUGAUGAGGCCGUUAGGCCGAA AUAGUCCU | 12946 |
| 3765 | UGGACACUA GCACUCUG | 3540 | CAGAGUGC CUGAUGAGGCCGUUAGGCCGAA AGUGUCCA | 12947 |
| 3771 | CUAGCACUC UGCUGGGC | 3541 | GCCCAGCA CUGAUGAGGCCGUUAGGCCGAA AGUGCUAG | 12948 |
| 3781 | GCUGGGCUC CCCCUUGC | 3542 | GCAAGGGG CUGAUGAGGCCGUUAGGCCGAA AGCCCAGC | 12949 |
| 3787 | CUCCCCCUU GCUGAAGC | 3543 | GCUUCAGC CUGAUGAGGCCGUUAGGCCGAA AGOGGGAG | 12950 |
| 3799 | GAAGCGGUU CACCUGGA | 3544 | UCCAGGUG CUGAUGAGGCCGUUAGGCCGAA ACCGCUUC | 12951 |
| 3800 | AAGCGGUUC ACCUGGAC | 3545 | GUCCAGGU CUGAUGAGGCCGUUAGGCCGAA AACCGCUU | 12952 |
| 3829 | CAAGGCCUC CAUGAAGA | 3546 | UCUUCAUG CUGAUGAGGCCGUUAGGCCGAA AGGCCUUG | 12953 |
| 3839 | AUGAAGAUA GACUUGAG | 3547 | CUCAAGUC CUGAUGAGGCCGUUAGGCCGAA AUCUUCAU | 12954 |
| 3844 | GAUAGACUU GAGAAUAG | 3548 | CUAUUCUC CUGAUGAGGCCGUUAGGCCGAA AGUCUAUC | 12955 |
| 3851 | UUGAGAAUA GCGAGUAA | 3549 | UUACUCGC CUGAUGAGGCCGUUAGGCCGAA AUUCUCAA | 12956 |
| 3858 | UAGCGAGUA AAAGCAAG | 3550 | CUUGCUUU CUGAUGAGGCCGUUAGGCCGAA ACUCGCUA | 12957 |
| 3878 | GCGGGACUU UCCGAUCU | 3551 | AGAUCGGA CUGAUGAGGCCGUUAGGCCGAA AGUCCCGC | 12958 |
| 3879 | CGGGACUUU CCGAUCUG | 3552 | CAGAUCGG CUGAUGAGGCCGUUAGGCCGAA AAGUCCCG | 12959 |
| 3880 | GGGACUUUC CGAUCUGC | 3553 | GCAGAUCG CUGAUGAGGCCGUUAGGCCGAA AAAGUCCC | 12960 |
| 3885 | UUUCCGAUC UGCCGAGG | 3554 | CCUCGGCA CUGAUGAGGCCGUUAGGCCGAA AUCGGAAA | 12961 |
| 3901 | GCCCAGCUU CUGCUUCU | 3555 | AGAAGCAG CUGAUGAGGCCGUUAGGCCGAA AGCUGGGC | 12962 |
| 3902 | CCCAGCUUC UGCUUCUC | 3556 | GAGAAGCA CUGAUGAGGCCGUUAGGCCGAA AAGCUGGG | 12963 |
| 3907 | CUUCUGCUU CUCCAGCU | 3557 | AGCUGGAG CUGAUGAGGCCGUUAGGCCGAA AGCAGAAG | 12964 |
| 3908 | UUCUGCUUC UCCACCUG | 3558 | CAGCUGGA CUGAUGAGGCCGUUAGGCCGAA AAGCAGAA | 12965 |
| 3910 | CUGCUUCUC CAGCUGUG | 3559 | CACAGCUG CUGAUGAGGCCGUUAGGCCGAA AGAAGCAG | 12966 |
| 3926 | GGCCACAUC AGGCCCGU | 3560 | ACGGGCCU CUGAUGAGGCCGUUAGGCCGAA AUGUGGCC | 12967 |
| 3949 | CGAUGAAUC UGAGCUGG | 3561 | CCAGCUCA CUGAUGAGGCCGUUAGGCCGAA AUUCAUCG | 12968 |
| 3967 | AAAGGAGUC CUGCUGUU | 3562 | AACAGCAG CUGAUGAGGCCGUUAGGCCGAA ACUCCUUU | 12969 |
| 3975 | CCUGCUGUU CUCCACCC | 3563 | GGCUGGAG CUGAUGAGGCCGUUAGGCCGAA ACAGCAGC | 12970 |
| 3976 | CUGCUGUUC UCCACCCC | 3564 | GGGGUGGA CUGAUGAGGCCGUUAGGCCGAA AACAGCAG | 12971 |
| 3978 | GCUGUUCUC CACCCCCA | 3565 | UGGGGGUG CUGAUGAGGCCGUUAGGCCGAA AGAACAGC | 12972 |
| 3991 | CCCAGACUA CAACUCCG | 3566 | CGGAGUUG CUGAUGAGGCCGUUAGGCCGAA AGUCUGGG | 12973 |
| 3997 | CUACAACUC CGUGGUGU | 3567 | ACACCACG CUGAUGAGGCCGUUAGGCCGAA AGUUGUAG | 12974 |
| 4006 | CGUGGUCUU GUACUCCU | 3568 | AGGAGUAC CUGAUGAGGCCGUUAGGCCGAA ACACCACO | 12975 |
| 4009 | GGUGUUGUA CUCCUCCC | 3569 | GGGAGGAG CUGAUGAGGCCGUUAGGCCGAA ACAACACC | 12976 |
| 4012 | GUUGUACUC CUCCCCGC | 3570 | GCGGGGAG CUGAUGAGGCCGUUAGGCCGAA AGUACAAC | 12977 |
| 4015 | GUACUCCUC CCCGCCCG | 3571 | CGGGCGGG CUGAUGAGGCCGUUAGGCCGAA AGGAGUAC | 12978 |
| 4027 | GCCCGCCUA AAGCUUCU | 3572 | AGAAGCUU CUGAUGAGGCCGUUAGGCCGAA AGGCGGGC | 12979 |
| 4033 | CUAAAGCUU CUCACCAG | 3573 | CUGGUGAG CUGAUGAGGCCGUUAGGCCGAA AGCUUUAG | 12980 |
| 4034 | UAAAGCUUC UCACCAGC | 3574 | GCUGGUGA CUGAUGAGGCCGUUAGGCCGAA AAGCUUUA | 12981 |
| 4036 | AAGCUUCUC ACCAGCCC | 3575 | GGGCUGGU CUGAUGAGGCCGUUAGGCCGAA AGAAGCUU | 12982 |
| 4066 | CUGACAGUA UUAUACAU | 3576 | AUGUAUAA CUGAUGAGGCCGUUAGGCCGAA ACUGUCAG | 12983 |
| 4068 | GACAGGAUG AUACAUCU | 3577 | AGAUGUAU CUGAUGAGGCCGUUAGGCCGAA AUACUGUC | 12984 |
| 4069 | ACAGUAUUA UACAUCUA | 3578 | UAGAUGUA CUGAUGAGGCCGUUAGGCCGAA AAUACUGU | 12985 |
| 4071 | AGUAUUAUA CAUCUAUG | 3579 | CAUAGAUG CUGAUGAGGCCGUUAGGCCGAA AUAAUACU | 12986 |
| 4075 | UUAUACAUC UAUGAGUU | 3580 | AACUCAUA CUGAUGAGGCCGUUAGGCCGAA AUGUAUAA | 12987 |
| 4077 | AUACAUCUA UGAGUUUA | 3581 | UAAACUCA CUGAUGAGGCCGUUAGGCCGAA AGAUGUAU | 12988 |
| 4083 | CUAUGAGUU UACACCUA | 3582 | GAGGUGHA CUGAUGAGGCCGUUAGGCCGAA ACUCAUAG | 12989 |
| 4084 | UAUGAGUUU ACACCUAU | 3583 | AUAGGUGU CUGAUGAGGCCGUUAGGCCGAA AACUCAUA | 12990 |
| 4085 | AUGAGUUGA CACCUAUU | 3584 | AAUAGGUG CUGAUGAGGCCGUUAGGCCGAA AAACUCAU | 12991 |
| 4091 | UUACACCUA UUCCGCUC | 3585 | GAGCGGAA CUGAUGAGGCCGUUAGGCCGAA AGGUGUAA | 12992 |
| 4093 | ACACCUAUU CCGCUCCA | 3586 | UGGAGCGG CUGAUGAGGCCGUUAGGCCGAA AUAGGUGU | 12993 |
| 4094 | CACCUAUUC CGCUCCAC | 3587 | GUGGAGCG CUGAUGAGGCCGUUAGGCCGAA AAUAGGUG | 12994 |
| 4099 | AUUCCGCUC CACAGGAG | 3588 | CUCCUGUG CUGAUGAGGCCGUUAGGCCGAA AGCGGAAU | 12995 |
| 4117 | CAGCUGCUU UUCGUGAC | 3589 | GUCACGAA CUGAUGAGGCCGUUAGGCCGAA AGCAGCUG | 12996 |
| 4118 | AGCUGCUUU UCGUGACC | 3590 | GGUCACGA CUGAUGAGGCCGUUAGGCCGAA AAGCAGCU | 12997 |
| 4119 | GCUGCUUUU CGUGACCU | 3591 | AGGUCACG CUGAUGAGGCCGUUAGGCCGAA AAAGCAGC | 12998 |
| 4120 | CUGCUUUUC GUGACCUU | 3592 | AAGGUCAC CUGAUGAGGCCGUUAGGCCGAA AAAAGCAG | 12999 |
| 4128 | CUUGACCUG UAAUCGUG | 3593 | CACGAUUA CUGAUGAGGCCGUUAGGCCGAA AGGUCACG | 13000 |
| 4129 | GUGACCUUU AAUCGUGC | 3594 | GCACGAUU CUGAUGAGGCCGUUAGGCCGAA AAGGUCAC | 13001 |
| 4130 | UGACCUUUA AUCGUGCU | 3595 | AGCACGAU CUGAUGAGGCCGUUAGGCCGAA AAAGGUCA | 13002 |
| 4133 | CCUUUAAUC GUGCUUUU | 3596 | AAAAGCAC CUGAUGAGGCCGUUAGGCCGAA AUUAAAGG | 13003 |
| 4139 | AUCGUGCUU UUUUGUUU | 3597 | AAACAAAA CUGAUGAGGCCGUUAGGCCGAA AGCACGAU | 13004 |
| 4140 | UCGUGCUUU UUUGUUUG | 3598 | AAAACAAA CUGAUGAGGCCGUUAGGCCGAA AAGCACGA | 13005 |
| 4141 | CGUGCUUUU UUGUUUUU | 3599 | AAAAACAA CUGAUGAGGCCGUUAGGCCGAA AAAGCACG | 13006 |
| 4142 | GUGCUUUUU UGUUUUUU | 3600 | AAAAAACA CUGAUGAGGCCGUUAGGCCGAA AAAAGCAC | 13007 |
| 4143 | UGCUUUUUU GUUUUUUG | 3601 | CAAAAAAC CUGAUGAGGCCGUUAGGCCGAA AAAAAGCA | 13008 |
| 4146 | UUUUGUGUG UUUUGUUU | 3602 | AAACAAAA CUGAUGAGGCCGUUAGGCCGAA ACAAAAAA | 13009 |
| 4147 | UUUUUGUUU UUUGUUUU | 3603 | AAAACAAA CUGAUGAGGCCGUUAGGCCGAA AACAAAAA | 13010 |
| 4148 | UUUGGUUUU UUGUUUUG | 3604 | CAAAACAA CUGAUGAGGCCGUUAGGCCGAA AAACAAAA | 13011 |
| 4149 | UUUGUUUUU UGUUUUGU | 3605 | ACAAAACA CUGAUGAGGCCGUUAGGCCGAA AAAACAAA | 13012 |
| 4150 | UUGUUUUUU GUUUUGUG | 3606 | AACAAAAC CUGAUGAGGCCGUUAGGCCGAA AAAAACAA | 13013 |
| 4153 | UUUUUUGUU UUGUUUGU | 3607 | ACAAACAA CUGAUGAGGCCGUUAGGCCGAA ACAAAAAA | 13014 |
| 4154 | UUUUGGUUU UGUUUGUU | 3608 | AACAAACA CUGAUGAGGCCGUUAGGCCGAA AACAAAAA | 13015 |
| 4155 | UUUUGUUUU GUUUGUUG | 3609 | CAACAAAC CUGAUGAGGCCGUUAGGCCGAA AAACCAAA | 13016 |
| 4158 | UGUUUUGUU UGUGGUUG | 3610 | CAACAACA CUGAUGAGGCCGUUAGGCCGAA ACAAAACA | 13017 |
| 4159 | GUUUUGUUU GUUGUUGC | 3611 | GCAACAAC CUGAUGAGGCCGUUAGGCCGAA AACAAAAC | 13018 |
| 4162 | UUGUUUGUU GUUGCUGU | 3612 | ACAGCAAC CUGAUGAGGCCGUUAGGCCGAA ACAAACAA | 13019 |
| 4165 | UUUGUUGUU GCUGUUUU | 3613 | AAAACAGC CUGAUGAGGCCGUUAGGCCGAA ACAACAAA | 13020 |
| 4171 | GUUGCUGUU UUGACUAA | 3614 | UUAGUCAA CUGAUGAGGCCGUUAGGCCGAA ACAGCAAC | 13021 |
| 4172 | UUGCUGUUU UGACUAAC | 3615 | GUUAGUCA CUGAUGAGGCCGUUAGGCCGAA AACAGCAA | 13022 |
| 4173 | UGCUGUUUU GACUAACA | 3616 | UGUUAGUC CUGAUGAGGCCGUUAGGCCGAA AAACAGCA | 13023 |
| 4178 | UUUUGACUA ACAAGAAU | 737 | AUUCUUGU CUGAUGAGGCCGUUAGGCCGAA AUUCAAAA | 10156 |
| 4189 | AAGAAUGUA ACCCCAGU | 3617 | ACUGGGGU CUGAUGAGGCCGUUAGCCCGAA ACAUUCUU | 13024 |
| 4198 | ACCCCAGUU AGUGACGU | 3618 | ACGUCACU CUGAUGAGGCCGUUAGGCCGAA ACUGGOGU | 13025 |
| 4199 | CCCCAGUUA UUGACGUG | 3619 | CACCUCAC CUGAUGAGGCCGUUAGGCCGAA AACUGGCC | 13026 |
| 4216 | UGAAGAAUA CUAUUGUU | 3620 | AACAAUAG CUGAUGAGGCCGUUAGGCCGAA AUUCUUCA | 13027 |
| 4219 | AGAAUACUA UUGUUAGA | 3621 | UCUAACAA CUGAUGAGGCCGUUAGGCCGAA AGUAUUCU | 13028 |
| 4221 | AAUACUAUU GUUAGAGA | 3622 | UCUCUAAC CUGAUGAGGCCGUUAGGCCGAA AUAGUAUU | 13029 |
| 4224 | ACUAUUGUU AGAGAAAU | 3623 | AUUUCUCU CUGAUGAGGCCGUUAGGCCGAA ACAAUAGU | 13030 |
| 4225 | CUAUUGUUA GACAAAUC | 3624 | GAUUUCUC CUGAUGAGGCCGUUAGGCCGAA AACAAUAG | 13031 |
| 4233 | AGAGAAAUC CCCCCCGC | 3625 | GCGGGGGU CUGAUGAGGCCGUUAGGCCGAA AUUUCUCU | 13032 |
| 4249 | CAAAGCCUC AGGGUAAC | 3626 | GUUACCCU CUGAUGAGCCGGUUAGGCCGAA AGGCUUUG | 13033 |
| 4255 | CUCAGGGUA ACCUGGAC | 3627 | GUCCAGGU CUGAUGAGGCCGUUAGGCCGAA ACCCUGAG | 13034 |
| 4282 | AGGUGCCUC UGGCGACC | 3628 | GGUCGCCA CUGAUGAGGCCGUUAGGCCGAA AGGCACCU | 13035 |
| 4323 | CCCACCCUC CCUGCAGC | 3629 | GCUGCAGG CUGAUGAGGCCGUUAGGCCGAA AGGGUGGG | 13036 |
| 4341 | GUGGGACUA GAGGCAGU | 3630 | ACUGCCUC CUGAUGAGGCCGUUAGGCCGAA AGUCCCAC | 13037 |
| 4350 | GAGGCAGUA AGCCCAUU | 3631 | AAUGGGCU CUGAUGAGGCCGUUAGGCCGAA ACUGCCUC | 13038 |
| 4358 | AAGCCCAUU AGCUCAUG | 3632 | CAUGAGCU CUGAUGAGGCCGUUAGGCCGAA AUGGGCUU | 13039 |
| 4359 | AGCCCAUUA GCUCAUGG | 3633 | CCAUGAGC CUGAUGAGGCCGUUAGGCCGAA AAUGGGCU | 13040 |
| 4363 | CAUUAGCUC AUGGCUGC | 3634 | GCAGCCAU CUGAUGAGGCCGUUAGGCCGAA AGCUAAUG | 13041 |
| 4387 | GACCUGCUC UGUCUCUC | 3635 | GAGAGACA CUGAUGAGGCCGUUAGGCCGAA AGCAGGUC | 13042 |
| 4391 | UGCUCUGUC UCUCUUAU | 3636 | AUAAGAGA CUGAUGAGGCCGUUAGGCCGAA ACAGACCA | 13043 |
| 4393 | CUCUGUCUC UCUUAUGG | 3637 | CCAUAAGA CUGAUGAGGCCGUUAGGCCGAA AGACAGAG | 13044 |
| 4395 | CUGUCUCUC UUAUGGAG | 3638 | CUCCAUAA CUGAUGAGGCCGUUAGGCCGAA AGAGACAG | 13045 |
| 4397 | GUCUCUCUU AUGGAGGA | 3639 | UCCUCCAU CUGAUGAGGCCGUUAGGCCGAA AGAGAGAC | 13046 |
| 4398 | UCUCUCUUA UGGAGGAA | 3640 | UUCCUCCA CUGAUGAGGCCGUUAGGCCGAA AAGAGAGA | 13047 |
| 4445 | AAAAGGCUU UGGGAUGC | 3641 | GCAUCCCA CUGAUGAGGCCGUUAGGCCGAA AGCCUUUU | 13048 |
| 4446 | AAAGGCUUU GGGAUGCG | 3642 | CGCAUCCC CUGAUGAGGCCGUUAGGCCGAA AAGCCUUU | 13049 |
| 4456 | GGAUGCGUC CGUCCUGU | 3643 | ACAGGACG CUGAUGAGGCCGUUAGGCCGAA ACGCAUCC | 13050 |
| 4460 | GCGUCCGUC CUGUGGAG | 3644 | CUCCACAG CUGAUGAGGCCGUUAGGCCGAA ACGGACGC | 13051 |
| 4487 | AGGGGGCUC CGCUAUGC | 3645 | GCAUAGCG CUGAUGAGGCCGUUAGGCCGAA AGCCCCCU | 13052 |
| 4492 | GCUCCGCUA UGCCACUU | 3646 | AAGUGGCA CUGAUGAGGCCGUUAGGCCGAA AGCGGAGC | 13053 |
| 4500 | AUGCCACUU CAGUGACU | 3647 | AGUCACUG CUGAUGAGGCCGUUAGGCCGAA AGUGGCAU | 13054 |
| 4501 | UGCCACUUC AGUGACUU | 3648 | AAGUCACU CUGAUGAGGCCGUUAGGCCGAA AAGUGGCA | 13055 |
| 4509 | CAGUGACUU CUCACUCC | 3649 | GGAGUGAG CUGAUGAGGCCGUUAGGCCGAA AGUCACUG | 13056 |
| 4510 | AGUGACUUC UCACUCCU | 3650 | AGGAGUGA CUGAUGAGGCCGUUAGGCCGAA AAGUCACU | 13057 |
| 4512 | UGACUUCUC ACUCCUGG | 3651 | CCAGGAGU CUGAUGAGGCCGUUAGGCCGAA AGAAGUCA | 13058 |
| 4516 | UUCUCACUC CUGGCCUC | 3652 | GAGGCCAG CUGAUGAGGCCGUUAGGCCGAA AGUGAGAA | 13059 |
| 4524 | CCUGGCCUC CGCUGUUU | 3653 | AAACAGCG CUGAUGAGGCCGUUAGGCCGAA AGGCCAGG | 13060 |
| 4531 | UCCGCUGUU UCGGGCCC | 3654 | GGGCCCGA CUGAUGAGGCCGUUAGGCCGAA ACAGCGGA | 13061 |
| 4532 | CCGCUGUUU CGGGCCCC | 3655 | GGGGCCCG CUGAUGAGGCCGUUAGGCCGAA AACAGCGG | 13062 |
| 4533 | CGCUGUUUC GGGCCCCC | 3656 | GGGGGCCC CUGAUGAGGCCGUUAGGCCGAA AAACAGCG | 13063 |
| 4543 | GGCCCCCUU CCAAGAGG | 3657 | CCUCUUGG CUGAUGAGGCCGUUAGGCCGAA AGGGGGCC | 13064 |
| 4544 | GCCCCCUUC CAAGAGGU | 3658 | ACCUCUUG CUGAUGAGGCCGUUAGGCCGAA AAGGGGGC | 13065 |
| 4553 | CAAGAGGUA UCAGACCA | 3659 | UGCUCUGA CUGAUGAGGCCGUUAGGCCGAA ACCUCUUG | 13066 |
| 4555 | AGAGGUAUC AGAGCAGA | 3660 | UCUGCUCU CUGAUGAGGCCGUUAGGCCGAA AUACCUCU | 13067 |
| 4577 | AGGGACGUU UCCUAGAC | 3661 | GUCUAGGA CUGAUGAGGCCGUUAGGCCGAA ACGUCCCU | 13068 |
| 4578 | GGGACGUUU CCUAGACC | 3662 | GGUCUAGG CUGAUGAGGCCGUUAGGCCGAA AACGUCCC | 13069 |
| 4579 | GGACGUUUC CUAGACCA | 3663 | UGGUCUAG CUGAUGAGGCCGUUAGGCCGAA AAACGUCC | 13070 |
| 4582 | CGUUUCCUA GACCAGOG | 3664 | CCCUGGUC CUGAUGAGGCCGUUAGGCCGAA AGGAAACG | 13071 |
| 4598 | GCACAUGUU CUCGGGAA | 3665 | UUCCCGAG CUGAUGAGGCCGUUAGGCCGAA ACAUGUGC | 13072 |
| 4599 | CACAUGGUC UCGGGAAC | 3666 | GUUCCCGA CUGAUGAGGCCGUUAGGCCGAA AACAUGUG | 13073 |
| 4601 | CAUGUUCUC GGGAACCA | 3667 | UGGUUCCC CUGAUGAGGCCGUUAGGCCGAA AGAACAUG | 13074 |
| 4614 | ACCACAGUU AAUCUUAA | 3668 | UUAAGAUU CUGAUGAGGCCGUUAGGCCGAA ACUGUGGU | 13075 |
| 4615 | CCACAGUUA AUCUUAAA | 3669 | UUUAAGAU CUGAUGAGGCCGUUAGGCCGAA AACUGUGG | 13076 |
| 4618 | CAGUUAAUC UUAAAUCU | 3670 | AGAUUUAA CUGAUGAGGCCGUUAGGCCGAA AUUAACUG | 13077 |
| 4620 | GUUAAUCUU AAAUCUUU | 3671 | AAAGAUUU CUGAUGAGGCCGUUAGGCCGAA AGAUUAAC | 13078 |
| 4621 | UUAAUCUUA AAUCUUUU | 3672 | AAAAGAUU CUGAUGAGGCCGUUAGGCCGAA AAGAUUAA | 13079 |
| 4625 | UCUUAAAUC UUUUCCCG | 3673 | CGGGAAAA CUGAUGAGGCCGUUAGGCCGAA AUUUAAGA | 13080 |
| 4627 | UUAAAUCUU UUCCCGGG | 3674 | CCCGGGAA CUGAUGAGGCCGUUAGGCCGAA AGAUUUAA | 13081 |
| 4628 | UAAAUCUUU UCCCGGGA | 3675 | UCCCGGGA CUGAUGAGGCCGUUAGGCCGAA AAGAUUUA | 13082 |
| 4629 | AAAUCUUUU CCCGGGAG | 3676 | CUCCCGGG CUGAUGAGGCCGUUAGGCCGAA AAAGAUUU | 13083 |
| 4630 | AAUCUUUUC CCGGGAGU | 3677 | ACUCCCGG CUGAUGAGGCCGUUAGGCCGAA AAAAGAUU | 13084 |
| 4639 | CCGGGAGUC GUCUGGUG | 3678 | CAACAGAA CUGAUGAGGCCGUUAGGCCGAA ACUCCCGG | 13085 |
| 4641 | GGGAGUCUU CUGGUGUC | 3679 | GACAACAG CUGAUGAGGCCGUUAGGCCGAA AGACUCCC | 13086 |
| 4642 | GGAGUCUUC UGUUGUCU | 3680 | AGACAACA CUGAUGAGGCCGUUAGGCCGAA AAGACUCC | 13087 |
| 4646 | UCUUCUGUU GUCUGUUU | 3681 | AAACAGAC CUGAUGAGGCCGUUAGGCCGAA ACAGAAGA | 13088 |
| 4649 | UCUGUUGUC UGUUUACC | 3682 | GGUAAACA CUGAUGAGGCCGUUAGGCCGAA ACAACAGA | 13089 |
| 4653 | UUGUCUGUU UACCAUCC | 3683 | GGAUGGUA CUGAUGAGGCCGUUAGGCCGAA ACAGACAA | 13090 |
| 4654 | UGUCUGUUU ACCAUCCA | 3684 | UGGAUGGU CUGAUGAGGCCGUUAGGCCGAA AACAGACA | 13091 |
| 4655 | GUCUGUUUA CCAUCCAA | 3685 | UUGGAUGG CUGAUGAGGCCGUUAGGCCGAA AAACAGAC | 13092 |
| 4660 | UGUACCAUC CAAAGCAU | 3686 | AUGCUUUG CUGAUGAGGCCGUUAGGCCGAA AUGGUAAA | 13093 |
| 4669 | CAAAGCAUA UUUAACAU | 3687 | AUGUUAAA CUGAUGAGGCCGUUAGGCCGAA AUGCUUUG | 13094 |
| 4671 | AAGCAUAUU UAACAUGU | 3688 | ACAUGUUA CUGAUGAGGCCGUUAGGCCGAA AUAUGCUU | 13095 |
| 4672 | AGCAUAUUU AACAUGUG | 3689 | CACAUGUU CUGAUGAGGCCGUUAGGCCGAA AAUAUGCU | 13096 |
| 4673 | GCAUAUUUA ACAUGUGU | 3690 | ACACAUGU CUGAUGAGGCCGUUAGGCCGAA AAAUAUGC | 13097 |
| 4682 | ACAUGUGUC AGUGGGGG | 3691 | CCCCCACU CUGAUGAGGCCGUUAGGCCGAA ACACAUGU | 13098 |
| 4698 | GUGGCGCUU GGCUUCUG | 3692 | CAGAAGCC CUGAUGAGGCCGUUAGGCCGAA AGCGCCAC | 13099 |
| 4703 | GCUUGGCUU CUGAGGCC | 3693 | GGCCUCAG CUGAUGAGGCCGUUAGGCCGAA AGCCAAGC | 13100 |
| 4704 | CUUGGCUUC UGAGGCCA | 3694 | UGGCCUCA CUGAUGAGGCCGUUAGGCCGAA AAGCCAAG | 13101 |
| 4720 | AGAGCCAUC AUCAGUUC | 3695 | GAACUGAU CUGAUGAGGCCGUUAGGCCGAA AUGGCUCU | 13102 |
| 4723 | GCCAUCAUC AGUUCCUC | 3696 | GAGGAACU CUGAUGAGGCCGUUAGGCCGAA AUGAUGGC | 13103 |
| 4727 | UCAUCAGUU CCUCUAGU | 3697 | ACUAGAGG CUGAUGAGGCCGUUAGGCCGAA ACUGAUGA | 13104 |
| 4728 | CAUCAGGUC CUCUAGUG | 3698 | CACUAGAG CUGAUGAGGCCGUUAGGCCGAA AACUGAUG | 13105 |
| 4731 | CAGUUCCUC UAGUGAGA | 3699 | UCUCACUA CUGAUGAGGCCGUUAGGCCGAA AGGAACUG | 13106 |
| 4733 | GUUCCUCUA GUGAGAUG | 3700 | CAUCUCAC CUGAUGAGGCCGUUAGGCCGAA AGAGGAAC | 13107 |
| 4745 | AGAUGCAUU GAGGUCAU | 3701 | AUGACCUC CUGAUGAGGCCGUUAGGCCGAA AUGCAUCU | 13108 |
| 4751 | AUUGAGGUC AUACCCAA | 3702 | UUGGGUAU CUGAUGAGGCCGUUAGGCCGAA ACCUCAAU | 13109 |
| 4754 | GAGGUCAUA CCCAAGCU | 3703 | AGCUUGGG CUGAUGAGGCCGUUAGGCCGAA AUGACCUC | 13110 |
| 4763 | CCCAAGCUU GCAGGCCU | 3704 | AGGCCUGC CUGAUGAGGCCGUUAGGCCGAA AGCUUGGG | 13111 |
| 4777 | CCUGACCUU CGCAUACU | 3705 | AGUAUGCG CUGAUGAGGCCGUUAGGCCGAA AGGUCAGG | 13112 |
| 4778 | CUGACCUUC GCAUACUG | 3706 | CAGUAUGC CUGAUGAGGCCGUUAGGCCGAA AAGGUCAG | 13113 |
| 4783 | CUUCGCAUA CUGCUCAC | 3707 | GUGAGCAG CUGAUGAGGCCGUUAGGCCGAA AUGCGAAG | 13114 |
| 4789 | AUACUGCUC ACGGGGAG | 3708 | CUCCCCGU CUGAUGAGGCCGUUAGGCCGAA AGCAGUAU | 13115 |
| 4799 | CGGGGAGUU AAGUGGUC | 3709 | GACCACUG CUGAUGAGGCCGUUAGGCCGAA ACUCCCCG | 13116 |
| 4800 | GGGGAGUUA AGUGGUCC | 3710 | GGACCACU CUGAUGAGGCCGUUAGGCCGAA AACUCCCC | 13117 |
| 4807 | UAAGUGGUC CAGUUUGG | 3711 | CCAAACUG CUGAUGAGGCCGUUAGGCCGAA ACCACUUA | 13118 |
| 4812 | GGUCCAGUU UGOCCUAG | 3712 | CUAGGCCA CUGAUGAGGCCGUUAGGCCGAA ACUGGACC | 13119 |
| 4813 | GUCCAGUUU GGCCUAGU | 3713 | ACUAGGCC CUGAUGAGGCCGUUAGGCCGAA AACUGGAC | 13120 |
| 4819 | UUUGGCCUA GUAAGGUU | 3714 | AACCUUAC CUGAUGAGGCCGUUAGGCCCAA AGGCCAAA | 13121 |
| 4822 | GGCCUAGUA AGGUUGCC | 3715 | GGCAACCU CUGAUGAGGCCGUUAGGCCGAA ACUAGGCC | 13122 |
| 4827 | AGUAAGGUU GCCUACUG | 3716 | CAGUAGOC CUGAUGAGGCCGUUAGGCCGAA ACCUGACH | 13123 |
| 4832 | GGUUGCCUA CUGAUGGG | 3717 | CCCAUCAG CUGAUGAGGCCGUUAGGCCGAA AGGCAACC | 13124 |
| 4843 | GAUGGGCUC AAAAGCCA | 3718 | UGGCUUUU CUGAUGAGGCCGUUAGGCCGAA AGCCCAUC | 13125 |
| 4855 | AGCCACAUU UUAAACAG | 3719 | CUGUUUAA CUGAUGAGGCCGUUAGGCCGAA AUGUGGCU | 13126 |
| 4856 | GCCACAUUU UAAACAGG | 3720 | CCUGUUUA CUGAUGAGGCCGUUAGGCCGAA AAUGUGGC | 13127 |
| 4857 | CCACAUUUU AAACAGGU | 3721 | ACCUGUUU CUGAUGAGGCCGUUAGGCCGAA AAAUGUGG | 13128 |
| 4858 | CACAUUUUA AACAGGUU | 3722 | AACCUGUU CUGAUGAGGCCGUUAGGCCGAA AAAAUGUG | 13129 |
| 4866 | AAACAGGUU UGAUCUCA | 3723 | UGAGAUAA CUGAUGAGGCCGUUAGGCCGAA ACCUGUUU | 13130 |
| 4867 | AACAGGUUU UAUCUCAA | 3724 | UUGAGAUA CUGAUGAGGCCGUUAGGCCGAA AACCUGUU | 13131 |
| 4868 | ACAGGUUUU AUCUCAAG | 3725 | CUUGAGAU CUGAUGAGGCCGUUAGGCCGAA AAACCUGU | 13132 |
| 4869 | CAGGUUUUA UCUCAAGU | 3726 | ACUUGAGA CUGAUGAGGCCGUUAGGCCGAA AAAACCUG | 13133 |
| 4871 | GGUUUUAUC UCAAGUAU | 3727 | AUACUUGA CUGAUGAGGCCGUUAGGCCGAA AUUAAACC | 13134 |
| 4873 | UUUUAUCUC AAGUAUUA | 3728 | UAAUACUU CUGAUGAGGCCGUUAGGCCGAA AGAUAAAA | 13135 |
| 4878 | UCUCAAGUA UUAAUAUA | 3729 | UAUAUUAA CUGAUGAGGCCGUUAGGCCGAA ACUUGAGA | 13136 |
| 4880 | UCAAGUAUU AAUAUAUA | 3730 | UAUAUAUU CUGAUGAGGCCGUUAGGCCGAA AUACUUGA | 13137 |
| 4881 | CAAGUAUUA AUAUAUAG | 3731 | CUAUAUAU CUGAUGAGGCCGUUAGGCCGAA AAUACUUG | 13138 |
| 4884 | GUAUUAAUA UAUAGACA | 3732 | UGUCUAGA CUGAUGAGGCCGUUAGGCCGAA AUUAAUAC | 13139 |
| 4886 | AUUAAUAUA UAGACAAG | 3733 | CUUGUCUA CUGAUGAGGCCGUUAGGCCGAA AUAUUAAU | 13140 |
| 4888 | UAAUAUAUA GACAAGAC | 3734 | GUCUUGUC CUGAUGAGGCCGUUAGGCCGAA AUAUAUUA | 13141 |
| 4900 | AAGACACUU AUGCAUUA | 3735 | UAAUGCAU CUGAUGAGGCCGUUAGGCCGAA AGUGUCUU | 13142 |
| 4901 | AGACACUUA UGCAUUAU | 3736 | AUAAUGCA CUGAUGAGGCCGUUAGGCCGAA AAGUGUCU | 13143 |
| 4907 | UUAUGCAUU AUCCUGUU | 3737 | AACAGGAU CUGAUGAGGCCGUUAGGCCGAA AUGCAUAA | 13144 |
| 4908 | UAUGCAUUA UCCUGUUU | 3738 | AAACAGGA CUGAUGAGGCCGUUAGGCCGAA AAUGCAUA | 13145 |
| 4910 | UGCAUUAUC CUGUUUUA | 3739 | UAAAACAG CUGAUGAGGCCGUUAGGCCGAA AUAAUGCA | 13146 |
| 4915 | UAUCCUGUU UUAUAUAU | 3740 | AUAUAUAA CUGAUGAGGCCGUUAGGCCGAA ACAGGAUA | 13147 |
| 4916 | AUCCUGUUU UAUAUAUC | 3741 | GAUAUAUA CUGAUGAGGCCGUUAGGCCGAA AACAGGAU | 13148 |
| 4917 | UCCUGUUUU AUAUAUCC | 3742 | GGAUAUAU CUGAUGAGGCCGUUAGGCCGAA AAACAGGA | 13149 |
| 4918 | CCUGUUUUA UAUAUCCA | 3743 | UGGAUAUA CUGAUGAGGCCGUUAGGCCGAA AAAACAGG | 13150 |
| 4920 | UGUUUUAUA UAUCCAAU | 3744 | AUUGGAUA CUGAUGAGGCCGUUAGGCCGAA AUAAAACA | 13151 |
| 4922 | UUUUAUAUA UCCAAUGA | 3745 | UCAUUGGA CUGAUGAGGCCGUUAGGCCGAA AUAUAAAA | 13152 |
| 4924 | UUAUAUAUC CAAUGAAU | 3746 | AUUCAUUG CUGAUGAGGCCGUUAGGCCGAA AUAUAUAA | 13153 |
| 4933 | CAAUGAAUA UAACUGGG | 3747 | CCCAGUUA CUGAUGAGGCCGUUAGGCCGAA AUUCAUUG | 13154 |
| 4935 | AUGAAUAUA ACUGGGGC | 3748 | GCCCCAGU CUGAUGAGGCCGUUAGGCCGAA AUAUUCAU | 13155 |
| 4948 | GGGCGAGUU AAGAGUCA | 3749 | UGACUCUU CUGAUGAGGCCGUUAGGCCGAA ACUCGCCC | 13156 |
| 4949 | GGCGAGUUA AGAGUCAU | 3750 | AUGACUCU CUGAUGAGGCCGUUAGGCCGAA AACUCGCC | 13157 |
| 4955 | UUAAGAGUC AUGGUCUA | 3751 | UAGACCAU CUGAUGAGGCCGUUAGGCCGAA ACUCUUAA | 13158 |
| 4961 | GUCAUGGUC UAGAAAAG | 3752 | CUUUUCUA CUGAUGAGGCCGUUAGGCCGAA ACCAUGAC | 13159 |
| 4963 | CAUGGUCUA GAAAAGGG | 3753 | CCCUUUUC CUGAUGAGGCCGUUAGGCCGAA AGACCAUG | 13160 |
| 4974 | AAAGGGGUU UCUCUGUA | 3754 | UACAGAGA CUGAUGAGGCCGUUAGGCCGAA ACCCCUUU | 13161 |
| 4975 | AAGGGGUUU CUCUGUAC | 3755 | GUACAGAG CUGAUGAGGCCGUUAGGCCGAA AACCCCUU | 13162 |
| 4976 | AGGGGUUUC UCUGUACC | 3756 | GGUACAGA CUGAUGAGGCCGUUAGGCCGAA AAACCCCU | 13163 |
| 4978 | GGGUUUCUC UGUACCCA | 3757 | UGGGUACA CUGAUGAGGCCGUUAGGCCGAA AGAAACCC | 13164 |
| 4982 | UUCUCUGUA CCCAAAUC | 3758 | GAUUUGGG CUGAUGAGGCCGUUAGGCCGAA ACAGAGAA | 13165 |
| 4990 | ACCCAAAUC GGGCUGGU | 3759 | ACCAGCCC CUGAUGAGGCCGUUAGGCCGAA AUUUGGGU | 13166 |
| 4999 | GGGCUGGUU GGACCAAG | 3760 | CUUGGUCC CUGAUGAGGCCGUUAGGCCGAA ACCAGCCC | 13167 |
| 5029 | AGAGUGGUG GUCCCAGC | 3761 | GCUGGGAC CUGAUGAGGCCGUUAGGCCGAA ACCACUCU | 13168 |
| 5032 | GUGGUUGUC CCAGCUAU | 3762 | AUAGCUGG CUGAUGAGGCCGUUAGGCCGAA ACAACCAC | 13169 |
| 5039 | UCCCAGCUA UAGUUACU | 3763 | AGUAACUA CUGAUGAGGCCGUUAGGCCGAA AGCUGGGA | 13170 |
| 5041 | CCAGCUAUA GUUACUAA | 3764 | UUAGUAAC CUGAUGAGGCCGUUAGGCCGAA AUAGCUGG | 13171 |
| 5044 | GCUAUAGUU ACUAAACU | 3765 | AGUUUAGU CUGAUGAGGCCGUUAGGCCGAA ACUAUAUC | 13172 |
| 5045 | CUAUAGUUA CUAAACUA | 3766 | UAGUUUAG CUGAUGAGGCCGUUAGGCCGAA AACUAUAG | 13173 |
| 5048 | UACUUACUA AACUACUC | 3767 | GAGUAGUU CUGAUGAGGCCGUUAGGCCGAA AGUAACUA | 13174 |
| 5053 | ACUAAACUA CUCACCCA | 3768 | UGGCUGAG CUGAUGAGGCCGUUAGUCCGAA AGUUUAGU | 13175 |
| 5056 | AAACUACUC ACCCAAAG | 3769 | CUUUGGGU CUGAUGAGCCCGUUAGGCCGAA AGUAGUUU | 13176 |
| 5066 | CCCAAAGUU GGGACCUC | 3770 | GAGGUCCC CUGAUGAGGCCGUUAGGCCGAA ACUUUGGG | 13177 |
| 5074 | UGGGACCUC ACUGGCUU | 3771 | AAGCCAGU CUGAUGAGGCCGUUAGGCCGAA AGGUCCCA | 13178 |
| 5082 | CACUGGCUU CUCUUUAC | 3772 | GUAAAGAG CUGAUGAGGCCGUUAGGCCGAA AUCCAGUC | 13179 |
| 5083 | ACUGUCUUC UCUUUACU | 3773 | AGUAAAGA CUGAUGAGGCCGUUAGGCCGAA AAGCCAGU | 13180 |
| 5085 | UGGCUUCUC UUUACUUC | 3774 | GAAGUAAA CUGAUGAGGCCGUUAGGCCGAA AGAAGCCA | 13181 |
| 5087 | GCUUCUCUU GACUUCAU | 3775 | AUGAAGUA CUGAUGAGGCCGUUAGGCCGAA AGAGAAGC | 13182 |
| 5088 | CUUCUCUUU ACUUCAUC | 3776 | GAUGAAGU CUGAUGAGGCCGUUAGGCCGAA AAGAGAAG | 13183 |
| 5089 | UUCUCUUUA CUUCAUCA | 3777 | UGAUGAAG CUGAUGAGGCCGUUAGGCCGAA AAAGAGAA | 13184 |
| 5092 | UCUUUACUU CAUCAUGG | 3778 | CCAUGAUG CUGAUGAGGCCGUUAGGCCGAA AGUAAAGA | 13185 |
| 5093 | CUUUACUUC AUCAUGGA | 3779 | UCCAUGAU CUGAUGAGGCCGUUAGGCCGAA AAGUAAAG | 13186 |
| 5096 | UACUUCAUC AUGGAUUU | 3780 | AAAUCCAU CUGAUGAGGCCGUUAGGCCGAA AUGAAGUA | 13187 |
| 5103 | UCAUGGAUU UCACCAUC | 3781 | GAUGGUGA CUGAUGAGGCCGUUAGGCCGAA AUCCAUGA | 13188 |
| 5104 | CAUGGAGUG CACCAUCC | 3782 | GGAUGGUG CUGAUGAGGCCGUUAGGCCGAA AAUCCAUG | 13189 |
| 5105 | AUGGAUUUC ACCAUCCC | 3783 | GGGAUGGU CUGAUGAGGCCGUUAGGCCGAA AAAUCCAU | 13190 |
| 5111 | UUCACCAUC CCAAGGCA | 3784 | UGCCUUGG CUGAUGAGGCCGUUAGGCCGAA AUGGUGAA | 13191 |
| 5122 | AAGGCAGUC UGAGAGGA | 3785 | UCCUCUCA CUGAUGAGGCCGUUAGGCCGAA ACUGCCUU | 13192 |
| 5134 | GAGGAGCUA AAGAGUAU | 3786 | AUACUCUU CUGAUGAGGCCGUUAGGCCGAA AGCUCCUC | 13193 |
| 5141 | UAAAGAGUA UCAGCCCA | 3787 | UGGGCUGA CUGAUGAGGCCGUUAGGCCGAA ACUCUUUA | 13194 |
| 5143 | AAGAGUAUC AGCCCAUA | 3788 | UAUGGGCU CUGAUGAGGCCGUUAGGCCGAA AUACUCUU | 13195 |
| 5151 | CAGCCCAUA UUUAUUAA | 3789 | UUAAUAAA CUGAUGAGGCCGUUAGGCCGAA AUGGGCUG | 13196 |
| 5153 | GCCCAUAUU UAUUAAGC | 3790 | GCUUAAUA CUGAUGAGGCCGUUAGGCCGAA AUAUGGGC | 13197 |
| 5154 | CCCAUAUUU AUUAAGCA | 3791 | UGCUUAAU CUGAUGAGGCCGUUAGGCCGAA AAUAUGGG | 13198 |
| 5155 | CCAUAUUUA UUAAGCAC | 3792 | GUGCUUAA CUGAUGAGGCCGUUAGGCCGAA AAAUAUGG | 13199 |
| 5157 | AUAUUUAUU AAGCACUU | 3793 | AAGUGCUU CUGAUGAGGCCGUUAGGCCGAA AUAUAUAU | 13200 |
| 5158 | UAUUUAUUA AGCACUUU | 3794 | AAAGUGCU CUGAUGAGGCCGUUAGGCCGAA AAUAAAUA | 13201 |
| 5165 | UAAGCACUU UAUGCUCC | 3795 | GGAGCAUA CUGAUGAGGCCGUUAGGCCGAA AGUGCUUA | 13202 |
| 5166 | AAGCACUUU AUGCUCCU | 3796 | AGGAGCAU CUGAUGAGGCCGUUAGGCCGAA AAGUGCUU | 13203 |
| 5167 | AGCACUUUA UGCUCCUU | 3797 | AAGGAGCA CUGAUGAGGCCGUUAGGCCGAA AAAGUGCU | 13204 |
| 5172 | UUUAUGCUC CUUGGCAC | 3798 | GUGCCAAG CUGAUGAGGCCGUUAGGCCGAA AGCAUAAA | 13205 |
| 5175 | AUGCUCCUU GGCACAGC | 3799 | GCUGUGCC CUGAUGAGGCCGUUAGGCCGAA AGGAGCAU | 13206 |
| 5195 | UGAUGUGUA AUUUAUGC | 3800 | GCAUAAAU CUGAUGAGGCCGUUAGGCCGAA ACACAUCA | 13207 |
| 5198 | UGUGUAAUU UAUGCAAG | 3801 | CUUGCAUA CUGAUGAGGCCGUUAGGCCGAA AUUACACA | 13208 |
| 5199 | GUGUAAUUU AUGCAAGC | 3802 | GCUUGCAU CUGAUGAGGCCGUUAGGCCGAA AAUUACAC | 13209 |
| 5200 | UGUAAUUUA UGCAAGCU | 3803 | AGCUUGCA CUGAUGAGGCCGUUAGGCCGAA AAAUUACA | 13210 |
| 5209 | UGCAAGCUC CCUCUCCA | 3804 | UGGAGAGG CUGAUGAGGCCGUUAGGCCGAA AGCUUGCA | 13211 |
| 5213 | AGCUCCCUC UCCAGCUA | 3805 | UAGCUGGA CUGAUGAGGCCGUUAGGCCGAA AGGGAGCU | 13212 |
| 5215 | CUCCCUCUC CAGCUAGG | 3806 | CCUAGCUG CUGAUGAGGCCGUUAGGCCGAA AGAGGGAG | 13213 |
| 5221 | CUCCAGCUA GUACUCAG | 3807 | CUGAGUCC CUGAUGAGGCCGUUAGGCCGAA ACCUGGAG | 13214 |
| 5227 | CUAGGACUC AGGAUAUU | 3808 | AAUAUCCU CUGAUGAGGCCGUUAGGCCGAA AGUCCUAG | 13215 |
| 5233 | CUCAGGAUA UUAGUCAA | 3809 | UUGACUAA CUGAUGAGGCCGUUAGGCCGAA AUCCUGAG | 13216 |
| 5235 | CAGGAUAUU AGUCAAUG | 3810 | CAUUGACU CUGAUGAGGCCGUUAGGCCGAA AUAUCCUG | 13217 |
| 5236 | AGGAUAUUA GUCAAUGA | 3811 | UCAUUGAC CUGAUGAGGCCGUUAGGCCGAA AAUAUCCU | 13218 |
| 5239 | AUAUUAGUC AAUGAGCC | 3812 | GGCUCAUU CUGAUGAGGCCGUUAGGCCGAA ACUAAUAU | 10358 |
| 5250 | UGAGCCAUC AAAAGGAA | 3813 | UUCCUUUU CUGAUGAGGCCGUUAGGCCGAA AUGGCUCA | 13219 |
| 5273 | AAAAACCUA UCUUAUUU | 3814 | AAAUAAGA CUGAUGAGGCCGUUAGGCCGAA AGGUUUUU | 13220 |
| 5275 | AAACCUAUC UUAUUUUC | 3815 | GAAAAUAA CUGAUGAGGCCGUUAGGCCGAA AUAGGUUU | 13221 |
| 5277 | ACCUAUCUU AUUUUCAU | 3816 | AUGAAAAU CUCAUCAGGCCGUUAGGCCGAA AGAUAGGU | 13222 |
| 5278 | CCUAUCUUA UUUUCAUC | 3817 | GAUGAAAA CUGAUUAGGCCGUUAGGCCGAA AAGAUAGG | 13223 |
| 5280 | UAUCUUAUU UUCAUCUG | 3818 | CAGAUGAA CUGAUGAGGCCGUUAGGCCGAA AUAAGAUA | 13224 |
| 5281 | AUCUUAUUU UCAUCUGU | 3819 | ACAGAUGA CUGAUGAGGCCGUUAGGCCGAA AAUAAGAU | 13225 |
| 5282 | UCUUAUUUU CAUCUGUU | 3820 | AACAGAUG CUGAUGAGGCCGUUAGGCCGAA AAAUAAGA | 13226 |
| 5283 | CUUAUUUUC AUCUGUUU | 3821 | AAACAGAU CUGAUGAGGCCGUUAGGCCGAA AAAAUAAG | 13227 |
| 5286 | AUUUUCAUC UGUUUCAU | 3822 | AUGAAACA CUGAUGAGGCCGUUAGGCCGAA AUGAAAAU | 13228 |
| 5290 | UCAUCUGUU UCAUACCU | 3823 | AGGUAUGA CUGAUGAGGCCGUUAGGCCGAA ACAGAUGA | 13229 |
| 5291 | CAUCUGUUU CAUACCUU | 3824 | AAGGUAUG CUGAUGAGGCCGUUAGGCCGAA AACAGAUG | 13230 |
| 5292 | AUCUGUUUC AUACCUUG | 3825 | CAAGGUAU CUGAUGAGGCCGUUAGGCCGAA AAACAGAU | 13231 |
| 5295 | UGUUUCAUA CCUUGUCU | 3826 | AGACAAGG CUGAUGAGGCCGUUAGGCCGAA AUGAAACA | 13232 |
| 5299 | UCAUACCUU GUCUGGGG | 3827 | CCCCAGAC CUGAUGAGGCCGUUAGGCCGAA AGGUAUGA | 13233 |
| 5302 | UACCUUGUC UGGGGUCU | 3828 | AGACCCCA CUGAUGAGGCCGUUAGGCCGAA ACAAGGUA | 13234 |
| 5309 | UCUGGGGUC UAAUGACG | 3829 | CGUCAUUA CUGAUGAGGCCGUUAGGCCGAA ACCCCAGA | 13235 |
| 5311 | UGGGGUCUA AUGACGAU | 3830 | AUCGUCAU CUGAUGAGGCCGUUAGGCCGAA AGACCCCA | 13236 |
| 5331 | AACAGGGUA GACAUGGG | 3831 | CCCAUGUC CUGAUGAGGCCGUUAGGCCGAA ACCCUGUU | 13237 |
| 5350 | GACAGGGUA GAAAAGGG | 3832 | CCCUUUUC CUGAUGAGGCCGUUAGGCCGAA ACCCUGUC | 13238 |
| 5367 | UGCCCGCUC UUUGGGGU | 3833 | ACCCCAAA CUGAUGAGGCCGUUAGGCCGAA AGCGGGCA | 13239 |
| 5369 | CCCGCUCUU UGGGGUCU | 3834 | AGACCCCA CUGAUGAGGCCGUUAGGCCGAA AGAGCGGG | 13240 |
| 5370 | CCGCUCUUU GGGGUCUA | 3835 | UAGACCCC CUGAUGAGGCCGUUAGGCCGAA AAGAGCGG | 13241 |
| 5376 | UUUGGGGUC UAGAGAUG | 3836 | CAUCUCUA CUGAUGAGGCCGUUAGGCCGAA ACCCCAAA | 13242 |
| 5378 | UGGGGUCUA GAGAUGAG | 3837 | CUCAUCUC CUGAUGAGGCCGUUAGGCCGAA AGACCCCA | 13243 |
| 5395 | CCCUGGGUC UCUAAAAU | 3838 | AUUUUAGA CUGAUGAGGCCGUUAGGCCGAA ACCCAGGG | 13244 |
| 5397 | CUGGGUCUC UAAAAUGG | 3839 | CCAUUUUA CUGAUGAGGCCGUUAGGCCGAA AGACCCAG | 13245 |
| 5399 | GGGUCUCUA AAAUGGCU | 3840 | AGCCAUUU CUGAUGAGGCCGUUAGGCCGAA AGAGACCC | 13246 |
| 5408 | AAAUGGCUC UCUUAGAA | 3841 | UUCUAAGA CUGAUGAGGCCGUUAGGCCGAA AGCCAUUU | 13247 |
| 5410 | AUGGCUCUC UUAGAAGU | 3842 | ACUUCUAA CUGAUGAGGCCGUUAGGCCGAA AGAGCCAU | 13248 |
| 5412 | GGCUCUCUU AGAAGUUG | 3843 | CAACUUCU CUGAUGAGGCCGUUAGGCCGAA AGAGAGCC | 13249 |
| 5413 | GCUCUCUUA GAAGUUGU | 3844 | ACAACUUC CUGAUGAGGCCGUUAGGCCGAA AAGAGAGC | 13250 |
| 5419 | UUAGAAGUU GUAUGUGC | 3845 | GCACAUAC CUGAUGAGGCCGUUAGGCCGAA ACUUCUAA | 13251 |
| 5422 | GAAGUUGUA UGUGCAAA | 3846 | UUUGCACA CUGAUGAGGCCGUUAGGCCGAA ACAACUUC | 13252 |
| 5432 | GUGCAAAUU AUGGUCUG | 3847 | CAGACCAU CUGAUGAGGCCGUUAGGCCGAA AUUUGCAC | 13253 |
| 5433 | UGCAAAUUA UGGUCUGU | 3848 | ACAGACCA CUGAUGAGGCCGUUAGGCCGAA AAUUUGCA | 13254 |
| 5438 | AUUAUGGUC UGUGUGCU | 3849 | AGCACACA CUGAUGAGGCCGUUAGGCCGAA ACCAUAAU | 13255 |
| 5447 | UGUGUGCUU AGGUCGUG | 3850 | CACGACCU CUGAUGAGGCCGUUAGGCCGAA AGCACACA | 13256 |
| 5448 | GUGUGCUUA GGUCGUGC | 3851 | GCACGACC CUGAUGAGGCCGUUAGGCCGAA AAGCACAC | 13257 |
| 5452 | GCUUAGGUC GUGCACAC | 3852 | GUGUGCAC CUGAUGAGGCCGUUAGGCCGAA ACCUAAGC | 13258 |
| 5475 | GAGCCGGUC ACAGCUGG | 3853 | CCAGCUGU CUGAUGAGGCCGUUAGGCCGAA ACCGGCUC | 13259 |
| 5497 | CGAUGAAUA GCUGCUUU | 3854 | AAAGCAGC CUGAUGAGGCCGUUAGGCCGAA AUUCAUCG | 13260 |
| 5504 | UAGCUGCUU UGGGAGAG | 3855 | CUCUCCCA CUGAUGAGGCCGUUAGGCCGAA AGCAGCUA | 13261 |
| 5505 | AGCUGCUUU GGGAGAGC | 3856 | GCUCUCCC CUGAUGAGGCCGUUAGGCCGAA AAGCAGCU | 13262 |
| 5524 | AGCAUGCUA GCCACUUA | 3857 | UAAGUGGC CUGAUGAGGCCGUUAGGCCGAA AGCAUGCU | 13263 |
| 5531 | UAGCCACUU AAUUCUCU | 3858 | AGAGAAUU CUGAUGAGGCCGUUAGGCCGAA AGUGGCUA | 13264 |
| 5532 | AGCCACUUA AUUCUCUG | 3859 | CAGAGAAU CUGAUGAGGCCGUUAGGCCGAA AAGUGGCU | 13265 |
| 5535 | CACUUAAUU CUCUGACC | 3860 | GGUCAGAG CUGAUGAGGCCGUUAGGCCGAA AUUAAGUG | 13266 |
| 5536 | ACUUAAUUC UCUGACOG | 3861 | CGGUCAGA CUGAUGAGGCCGUUAGGCCGAA AAUUAAGU | 13267 |
| 5538 | UUAAUUCUC UGACCOOG | 3862 | CCCGGUCA CUGAUGAGGCCGUUAGGCCGAA AGAAUUAA | 13268 |
| 5554 | GCCAGCAUC AUGGGUAC | 3863 | GUACCCAU CUGAUGAGGCCGUUAGGCCGAA AUGCUGGC | 13269 |
| 5561 | UCAUGGGUA CCUGCUCC | 3864 | GGAGCAGG CUGAUGAGGCCGUUAGGCCGAA ACCCAUGA | 13270 |
| 5568 | UACCUGCUC CCCUGUGU | 3865 | ACACAGGG CUGAUGAGGCCGUUAGGCCGAA AGCAGGUA | 13271 |
| 5577 | CCCUGUGUA CCCCAUCC | 3866 | GGAUGGGG CUGAUGAGGCCGUUAGGCCGAA ACACAGGG | 13272 |
| 5584 | UACCCCAUC CUUAAGGU | 3867 | ACCUUAAG CUGAUGAGGCCGUUAGGCCGAA AUGGGGUA | 13273 |
| 5587 | CCCAUCCUU AAGGUUUU | 3868 | AAAACCUU CUGAUGAGGCCGUUAGGCCGAA AGGAUCGG | 13274 |
| 5588 | CCAUCCUUA AGCUUUUC | 3869 | GAAAACCU CUGAUGAGGCCGUUAGGCCGAA AAGGAUGG | 13275 |
| 5593 | CUUAAGGUU UUCUGUCU | 3870 | AGACAGAA CUGAUGAGGCCGUUAGGCCGAA ACCUUAAG | 13276 |
| 5594 | UUAAGGUUU UCUGUCUG | 3871 | CAGACAGA CUGAUGAGGCCGUUAGGCCGAA AACCUUAA | 13277 |
| 5595 | UAAGGUUUU CUGUCUGA | 3872 | UCAGACAG CUGAUGAGGCCGUUAGGCCGAA AAACCUUA | 13278 |
| 5596 | AAGGUUUUC UGUCUGAU | 3873 | AUCAGACA CUGAUGAGGCCGUUAGGCCGAA AAAACCUU | 13279 |
| 5600 | UUUUCUGUC UGAUGAGA | 3874 | UCUCAUCA CUGAUGAGGCCGUUAGGCCGAA ACAGAAAA | 13280 |
| 5627 | AGUGCAAUC CCCACUGA | 3875 | UCAGUGGG CUGAUGAGGCCGUUAGGCCGAA AUUGCACU | 13281 |
| 5660 | CUGUGOCUC UUGGUGCA | 3876 | UGCACCAA CUGAUGAGGCCGUUAGGCCGAA AGCCACAG | 13282 |
| 5662 | GUGGCUCUU GGUGCACU | 3877 | AGUGCACC CUGAUGAGGCCGUUAGGCCGAA AGAGCCAC | 13283 |
| 5671 | GGUGCACUC ACCAGCCA | 3878 | UGGCUGGU CUGAUGAGGCCGUUAGGCCGAA AGUGCACC | 13284 |
| 5685 | CCAGGACUA GACAAGUA | 3879 | UACUUGUC CUGAUGAGGCCGUUAGGCCGAA AGUCCUGG | 13285 |
| 5693 | AGACAAGUA GGAAAGGG | 3880 | CCCUUUCC CUGAUGAGGCCGUUAGGCCGAA ACUUGUCU | 13286 |
| 5704 | AAAGGGCUU CUAGCCAC | 3881 | GUGGCUAG CUGAUGAGGCCGUUAGGCCGAA AGCCCUUU | 13287 |
| 5705 | AAGGGCUUC UAGCCACA | 3882 | UGUGGCUA CUGAUGAGGCCGUUAGGCCGAA AAGCCCUU | 13288 |
| 5707 | GGGCUUCUA GCCACACU | 3883 | AGUGUGGC CUGAUGAGGCCGUUAGGCCGAA AGAAGCCC | 13289 |
| 5731 | AAGAAAAUC AGGUAGGG | 3884 | CCCUACCU CUGAUGAGGCCGUUAGGCCGAA AUUUUCUU | 13290 |
| 5736 | AAUCAGGUA GGGCUGGC | 3885 | GCCAGCCC CUGAUGAGGCCGUUAGGCCGAA ACCUGAUU | 13291 |
| 5754 | AAAGACAUC UUUGUCCA | 3886 | UGGACAAA CUGAUGAGGCCGUUAGGCCGAA AUGUCUUU | 13292 |
| 5756 | AGACAUCUU UGUCCAUU | 3887 | AAUGGACA CUGAUGAGGCCGUUAGGCCGAA AGAUGUCU | 13293 |
| 5757 | GACAUCUUU GUCCAUUC | 3888 | GAAUGGAC CUGAUGAGGCCGUUAGGCCGAA AAGAUGUC | 13294 |
| 5760 | AUCUUUGUC CAUUCGCA | 3889 | UGCGAAUG CUGAUGAGGCCGUUAGGCCGAA ACAAAGAU | 13295 |
| 5764 | UUGUCCAUU CGCAAAAG | 3890 | CUUUUGCG CUGAUGAGGCCGUUAGGCCGAA AUGGACAA | 13296 |
| 5765 | UGUCCAUUC GCAAAAGC | 3891 | GCUUUUGC CUGAUGAGGCCGUUAGGCCGAA AAUGGACA | 13297 |
| 5775 | CAAAAGCUC UUGUCGGC | 3892 | GCCGACAA CUGAUGAGGCCGUUAGGCCGAA AGCUUUUG | 13298 |
| 5777 | AAAGCUCUU GUCGGCUG | 3893 | CAGCCGAC CUGAUGAGGCCGUUAGGCCGAA AGAGCUUU | 13299 |
| 5780 | GCUCUUGUC GGCUGCAG | 3894 | CUGCAGCC CUGAUGAGGCCGUUAGGCCGAA ACAAGAGC | 13300 |
| 5794 | CAGUGUGUA AGUCAGGC | 3895 | GCCUGACU CUGAUGAGGCCGUUAGGCCGAA ACACACUG | 13301 |
| 5798 | GUGUAAGUC AGGCGAUG | 3896 | CAUCGCCU CUGAUGAGGCCGUUAGGCCGAA ACUUACAC | 13302 |
| 5818 | CAGAGGCUA CCAGAGAA | 3897 | UUCUCUGG CUGAUGAGGCCGUUAGGCCGAA AGCCUCUG | 13303 |
| 5852 | CCUGAGGUU UCUCAUCC | 3898 | GGAUGAGA CUGAUGAGGCCGUUAGGCCGAA ACCUCAGG | 13304 |
| 5853 | CUGAGGUUU CUCAUCCA | 3899 | UGGAUGAG CUGAUGAGGCCGUUAGGCCGAA AACCUCAG | 13305 |
| 5854 | UGAGGUUUC UCAUCCAG | 3900 | CUGGAUGA CUGAUGAGGCCGUUAGGCCGAA AAACCUCA | 13306 |
| 5856 | AGGUUUCUC AUCCAGAU | 3901 | AUCUGGAU CUGAUGAGGCCGUUAGGCCGAA AGAAACCU | 13307 |
| 5859 | UUUCUCAUC CAGAUAUC | 3902 | GAUAUCUG CUGAUGAGGCCGUUAGGCCGAA AUGAGAAA | 13308 |
| 5865 | AUCCAGAUA UCCAGCAA | 3903 | UUGCUGGA CUGAUGAGGCCGUUAGGCCGAA AUCUGGAU | 13309 |
| 5867 | CCAGAUAUC CAGCAAUU | 3904 | AAUUGCUG CUGAUGAGGCCGUUAGGCCGAA AUAUCUGG | 13310 |
| 5875 | CCAGCAAUU GGGGGGUG | 3905 | CACCCCCC CUGAUGAGGCCGUUAGGCCGAA AUUGCUGG | 13311 |
| 5896 | AAGACCAUA GAUGGUCC | 3906 | GGACCAUC CUGAUGAGGCCGUUAGGCCGAA AUGGUCUU | 13312 |
| 5903 | UAGAUGGUC CUGUAUUA | 3907 | UAAUACAG CUGAUGAGGCCGUUAGGCCGAA ACCAUCUA | 13313 |
| 5908 | GGUCCUGUA UUAUUCCG | 3908 | CGGAAUAA CUGAUGAGGCCGUUAGGCCGAA ACAGGACC | 13314 |
| 5910 | UCCUGUAUU AUUCCGAU | 3909 | AUCGGAAU CUGAUGAGGCCGUUAGGCCGAA AUACAGGA | 13315 |
| 5911 | CCUGUAUUA UUCCGAUU | 3910 | AAUCGGAA CUGAUGAGGCCGUUAGGCCGAA AAUACAGG | 13316 |
| 5913 | UGUAUUAUU CCGAUUUU | 3911 | AAAAUCGG CUGAUGAGGCCGUUAGGCCGAA AUAAUACA | 13317 |
| 5914 | GUAUUAUUC CGAUUUUA | 3912 | UAAAAUCG CUGAUGAGGCCGUUAGGCCGAA AAUAAUAC | 13318 |
| 5919 | AUUCCGAUU UUAAUAAU | 3913 | AUUAUUAA CUGAUGAGGCCGUUAGGCCGAA AUCGGAAU | 13319 |
| 5920 | UUCCGAUUU UAAUAAUC | 3914 | GAUUAUUA CUGAUGAGGCCGUUAGGCCGAA AAUCGGAA | 13320 |
| 5921 | UCCGAUUUU AAUAAUCU | 3915 | AGAUUAUU CUGAUGAGGCCGUUAGGCCGAA AAAUCGGA | 13321 |
| 5922 | CCGAUUUUA AUAAUCUA | 3916 | UAGAUUAU CUGAUGAGGCCGUUAGGCCGAA AAAAUCGG | 13322 |
| 5925 | AUUUUAAUA AUCUAAUU | 3917 | AAUUAGAU CUGAUGAGGCCGUUAGGCCGAA AUUAAAAU | 13323 |
| 5928 | UUAAUAAUC UAAUUCGU | 3918 | ACGAAUUA CUGAUGAGGCCGUUAGGCCGAA AUUAUUAA | 13324 |
| 5930 | AAUAAUCUA AUUCGUGA | 3919 | UCACGAAU CUGAUGAGGCCGUUAGGCCGAA AGAUUAUU | 13325 |
| 5933 | AAUCUAAUU CGUGAUCA | 3920 | UGAUCACG CUGAUGAGGCCGUUAGGCCGAA AUUAGAUU | 13326 |
| 5934 | AUCUAAUUC GUGAUCAU | 3921 | AUGAUCAC CUGAUGAGGCCGUUAGGCCGAA AAUUAGAU | 13327 |
| 5940 | UUCGUGAUC AUUAAGAG | 3922 | CUCUUAAU CUGAUGAGGCCGUUAGGCCGAA AUCACGAA | 13328 |
| 5943 | GUGAUCAUU AAGAGACU | 3923 | AGUCUCUU CUGAUGAGGCCGUUAGGCCGAA AUGAUCAC | 13329 |
| 5944 | UGAUCAUUA AGAGACUU | 3924 | AAGUCUCU CUGAUGAGGCCGUUAGGCCGAA AAUGAUCA | 13330 |
| 5952 | AAGAGACUU UAGUAAAU | 3925 | AUUUACUA CUGAUGAGGCCGUUAGGCCGAA AGUCUCUU | 13331 |
| 5953 | AGAGACUUU ACUAAAUG | 3926 | CAUUUACU CUGAUGAGGCCGUUAGGCCGAA PAGUCUCU | 13332 |
| 5954 | GAGACUUUA GUAAAUGU | 3927 | ACAUGUAC CUGAUGAGGCCGUUAGGCCGAA AAAGUCUC | 13333 |
| 5957 | ACUUUAGUA AAUGUCCC | 3928 | GGGACAUU CUGAUGAGGCCGUUAGGCCGAA ACUAAAGU | 13334 |
| 5963 | GUAAAUGUC CCUUUUCC | 3929 | GGAAAAGG CUGAUGAGGCCGUUAGGCCGAA ACAUUUAC | 13335 |
| 5967 | AUGUCCCUU UUCCCACA | 3930 | UGUGGGAA CUGAUGAGGCCGUUAGGCCGAA AGGGACAU | 13336 |
| 5968 | UGUCCCUUU UCCCACAA | 3931 | UUGUGGGA CUGAUGAGGCCGUUAGGCCGAA AAGGGACA | 13337 |
| 5969 | GUCCCUUUU CCCACAAA | 3932 | UUUGUGGG CUGAUGAGGCCGUUAGGCCGAA AAAGGGAC | 13338 |
| 5970 | UCCCUUUUC CCACAAAA | 3933 | UUUUGUGG CUGAUGAGGCCGUUAGGCCGAA AAAAGGGA | 13339 |
| 5981 | ACAAAAGUA AAGAAAAG | 3934 | CUUUUCUU CUGAUGAGGCCGUUAGGCCGAA ACUUUUGU | 13340 |
| 5992 | GAAAAGCUA UCGGGAUU | 3935 | AAUCCCGA CUGAUGAGGCCGUUAGGCCGAA AGCUUUUC | 13341 |
| 5994 | AAAGCUAUC GGGAUUCU | 3936 | AGAAUCCC CUGAUGAGGCCGUUAGGCCGAA AUAGCUUU | 13342 |
| 6000 | AUCGGGAUU CUCUGGUU | 3937 | AACCAGAG CUGAUGAGGCCGUUAGGCCGAA AUCCCGAU | 13343 |
| 6001 | UCGGGAUUC UCUGGUUC | 3938 | GAACCAGA CUCAUGAGGCCGUUAGGCCGAA AAUCCCGA | 13344 |
| 6003 | GGGAUUCUC UGGUUCUG | 3939 | CAGAACCA CUGAUGAGGCCGUUAGGCCGAA AGAAUCCC | 13345 |
| 6008 | UCUCUGGUU CUGCUUAA | 3940 | UUAAGCAG CUGAUGAGGCCGUUAGGCCGAA ACCAGAGA | 13346 |
| 6009 | CUCUGGUUC UGCUUAAA | 3941 | UUUAAGCA CUGAUGAGGCCGUUAGGCCGAA AACCAGAG | 13347 |
| 6014 | GUUCUGCUU AAAGACUU | 3942 | AAGUCUUU CUGAUGAGGCCGUUAGGCCGAA AGCAGAAC | 13348 |
| 6015 | UUCUGCUUA AAGACUUA | 3943 | UAAGUCUU CUGAUGAGGCCGUUAGGCCGAA AAGCAGAA | 13349 |
| 6022 | UAAAGACUU AGCUUUGG | 3944 | CCAAAGCU CUGAUGAGGCCGUUAGGCCGAA AGUCUUUA | 13350 |
| 6023 | AAAGACUUA GCUUUGGA | 3945 | UCCAAAGC CUGAUGAGGCCGUUAGGCCGAA AAGUCUUU | 13351 |
| 6027 | ACUUAGCUU UGGAGCCU | 3946 | AGOCUCCA CUGAUGAGGCCGUUAGGCCGAA AGCUAAGU | 13352 |
| 6028 | CUUAGCUUU GGAGCCUA | 3947 | UAGGCUCC CUGAUGAGGCCGUUAGGCCGAA AAGCUAAG | 13353 |
| 6036 | UGGAGCCUA UGAAACUU | 3948 | AACUUUCA CUGAUGAGGCCGUUAGGCCGAA AGGCUCCA | 13354 |
| 6044 | AUGAAAGUU GAUCAGCC | 3949 | GGCUGAUC CUGAUGAGGCCGUUAGGCCGAA ACUUUCAU | 13355 |

Where “X” represents stem II region of a HH ribozyme (Hertel et al., 1992 Nucleic Acids Res. 20 3252). The length of stem II may be ≧ 2 base-pairs.
Underlined region can be any X sequence or linker, as described herein.

TABLE IX
Mouse flt1 VEGF Receptor—Hairpin Ribozyme and Substrate Sequence

| | | Seq ID | | Seq ID |
|---|---|---|---|---|
| Pos | Substrate | No | HP Ribozyme | No |
| 33 | AUGGUCA GCU GCUGGGAC | 3950 | GUCCCAGC AGAA GACCAU ACCAGAGAAACACACGUUGUGGUACAUUACCUGGUA | 13356 |
| 36 | GUCAGCU GCU GGGACACC | 3951 | GGUGUCCC AGAA GCUGAC ACCAGAGAAACACACGUUGUGGUACAUUACCUGGUA | 13357 |
| 50 | CACCGCG GUC UUGCCUUA | 3952 | UAAGGCAA AGAA GCGGUG ACCAGAGAAACACACGUUGUGGUACAUUACCUGGUA | 13358 |
| 67 | ACGCGCU GCU CGGGUGUC | 3953 | GACACCCG AGAA GCGCGU ACCAGAGAAACACACGUUGUGGUACAUUACCUGGUA | 13359 |
| 79 | GGUGUCU GCU UCUCACAG | 3954 | CUGUGAGA AGAA GACACC ACCAGAGAAACACACGUUGUGGUACAUUACCUGGUA | 13360 |
| 166 | CAGGCCA GAC UCUCUUUC | 3955 | GAAAGAGA AGAA GGCCUG ACCAGAGAAACACACGUUGUGGUACAUUACCUGGUA | 13361 |
| 197 | GGAGGCA GCC CACUCAUG | 3956 | CAUGAGUG AGAA GCCUCC ACCAGAGAAACACACGUUGUGGUACAUUACCUGGUA | 13362 |
| 214 | GGUCUCU GCC CACGACCG | 3957 | CGGUCGUG AGAA GAGACC ACCAGAGAAACACACGUUGUGGUACAUUACCUGGUA | 13363 |
| 266 | CCCAUCG GCC UGUGGGAG | 3958 | CUCCCACA AGAA GAUGGG ACCAGAGAAACACACGUUGUGGUACAUUACCUGGUA | 13364 |
| 487 | GAAGACA GCU CAUCAUCC | 3959 | GGAUGAUG AGAA GUCUUC ACCAGAGAAACACACGUUGUGGUACAUUACCUGGUA | 13365 |
| 501 | AUCCCCU GCC GGGUGACG | 3960 | CGUCACCC AGAA GGGGAU ACCAGAGAAACACACGUUGUGGUACAUUACCUGGUA | 13366 |
| 566 | UACCCCU GAU GGGCAAAG | 3961 | CUUUGCCC AGAA GGGGUA ACCAGAGAAACACACGUUGUGGUACAUUACCUGGUA | 13367 |
| 640 | UAGGACU GCU GAACUGCG | 3962 | CGCAGUUC AGAA GUCCUA ACCAGAGAAACACACGUUGUGGUACAUUACCUGGUA | 13368 |
| 691 | ACUAUCU GAC CCAUCGGC | 3963 | GCCGAUGG AGAA GAUAGU ACCAGAGAAACACACGUUGUGGUACAUUACCUGGUA | 13369 |
| 703 | AUCGGCA GAC CAAUACAA | 3964 | UUGUAUUG AGAA GCCGAU ACCAGAGAAACACACGUUGUGGUACAUUACCUGGUA | 13370 |
| 736 | UACGCCC GCC GAGCCCAG | 3965 | CUGGGCUC AGAA GGCGUA ACCAGAGAAACACACGUUGUGGUACAUUACCUGGUA | 13371 |
| 754 | UGAGACU GCU CCACGGGC | 3966 | GCCCGUGG AGAA GUCUCA ACCAGAGAAACACACGUUGUGGUACAUUACCUGGUA | 13372 |
| 766 | ACGGGCA GAC UCUUGUCC | 3967 | GGACAAGA AGAA GCCCGU ACCAGAGAAACACACGUUGUGGUACAUUACCUGGUA | 13373 |
| 871 | GGCAGCG GAU UGACCGGA | 3968 | UCCGGUCA AGAA GCUGCC ACCAGAGAAACACACGUUGUGGUACAUUACCUGGUA | 13374 |
| 960 | UACACCU GUC GCGUGAAG | 3969 | CUUCACGC AGAA GGUGUA ACCAGAGAAACACACGUUGUGGUACAUUACCUGGUA | 13375 |
| 988 | CGUUCCA GUC UUUCAACA | 3970 | UGUUGAAA AGAA GGAACG ACCAGAGAAACACACGUUGUGGUACAUUACCUGGUA | 13376 |
| 1051 | GGAAGCA GCC GGUGCAGG | 3971 | CCUGCACC AGAA GCUUCC ACCAGAGAAACACACGUUGUGGUACAUUACCUGGUA | 13377 |
| 1081 | GAAGACG GUC CUAUCGGC | 3972 | GCCGAUAG AGAA GUCUUC ACCAGAGAAACACACGUUGUGGUACAUUACCUGGUA | 13378 |
| 1090 | CCUAUCG GCU GUCCAUGA | 3973 | UCAUGGAC AGAA GAUAGG ACCAGAGAAACACACGUUGUGGUACAUUACCUGGUA | 13379 |
| 1093 | AUCGGCU GUC CAUGAAAG | 3974 | CUUUCAUG AGAA GCCGAU ACCAGAGAAACACACGUUGUGGUACAUUACCUGGUA | 13380 |
| 1169 | GAAGUCU GCU CGCUAUUU | 3975 | AAAUAGCG AGAA GACUUC ACCAGAGAAACACACGUUGUGGUACAUUACCUGGUA | 13381 |
| 1315 | AACCUCA GAU CUACGAAA | 3976 | UUUCGUAG AGAA GAGGUU ACCAGAGAAACACACGUUGUGGUACAUUACCUGGUA | 13382 |
| 1363 | UCUAUCC GCU GGGCAGCA | 3977 | UGCUGCCC AGAA GAUAGA ACCAGAGAAACACACGUUGUGGUACAUUACCUGGUA | 13383 |
| 1604 | GGUGGCU GAC UCUCAGAC | 3978 | GUCUGAGA AGAA GCCACC ACCAGAGAAACACACGUUGUGGUACAUUACCUGGUA | 13384 |
| 1612 | ACUCUCA GAC CCCUGGAA | 3979 | UUCCAGGG AGAA GAGAGU ACCAGAGAAACACACGUUGUGGUACAUUACCUGGUA | 13385 |
| 1629 | AUCUACA GCU GCCGGGCC | 3980 | GGCCCGGC AGAA GUAGAU ACCAGAGAAACACACGUUGUGGUACAUUACCUGGUA | 13386 |
| 1632 | UACAGCU GCC GGGCCUUC | 3981 | GAAGGCCC AGAA GCUGUA ACCAGAGAAACACACGUUGUGGUACAUUACCUGGUA | 13387 |
| 1688 | UGUCACA GAU GUGCCGAA | 3982 | CUGGGCAC AGAA GUGACA ACCAGAGAAACACACGUUGUGGUACAUUACCUGGUA | 13388 |
| 1730 | GAUGCCA GCC GAAGGAGA | 3983 | UCUCCUUC AGAA GGCAUC ACCAGAGAAACACACGUUGUGGUACAUUACCUGGUA | 13389 |
| 1753 | UGAAACU GUC CUGUGUGG | 3984 | CCACACAG AGAA GUUUCA ACCAGAGAAACACACGUUGUGGUACAUUACCUGGUA | 13390 |
| 2017 | CACACCU GCU UCAAAACC | 3985 | GGUUUUGA AGAA GGUGUG ACCAGAGAAACACACGUUGUGGUACAUUACCUGGUA | 13391 |
| 2101 | CGCCUCA GAU CACUUGGU | 3988 | ACCAAGUG AGAA GAGGCG ACCAGAGAAACACACGUUGUGGUACAUUACCUGGUA | 13392 |
| 2176 | GCACGCU GUU UAUUGAAA | 1441 | UUUCAAUA AGAA GCGUGC ACCAGAGAAACACACGUUGUGGUACAUUACCUGGUA | 10860 |
| 2258 | AAGCGCA GCC UACCUCAC | 3987 | GUGAGGUA AGAA GCGCUU ACCAGAGAAACACACGUUGUGGUACAUUACCUGGUA | 13393 |
| 2305 | UGGAGCU GAU CACGCUCA | 3988 | UGAGCGUG AGAA GCUCCA ACCAGAGAAACACACGUUGUGGUACAUUACCUGGUA | 13394 |
| 2383 | UGAAGCG GUC UUCUUCCG | 3989 | CGGAAGAA AGAA GCUUCA ACCAGAGAAACACACGUUGUGGUACAUUACCUGGUA | 13395 |
| 2405 | AAAGACA GAC UACCUGUC | 3990 | GACAGGUA AGAA GUCUUU ACCAGAGAAACACACGUUGUGGUACAUUACCUGGUA | 13396 |
| 2432 | GGACCCA GAU GAAGUUCC | 1445 | GGAACUUC AGAA GGGUCC ACCAGAGAAACACACGUUGUGGUACAUUACCUGGUA | 10864 |
| 2464 | GUGAACG GCU GCCCUAUG | 3991 | CAUAGGGC AGAA GUUCAC ACCAGAGAAACACACGUUGUGGUACAUUACCUGGUA | 13397 |
| 2467 | AACGGCU GCC CUAUGAUG | 3992 | CAUCAUAG AGAA GCCGUU ACCAGAGAAACACACGUUGUGGUACAUUACCUGGUA | 13398 |
| 2592 | CCCACCU GCC GGACUGUG | 3993 | CACAGUCC AGAA GGUGGG ACCAGAGAAACACACGUUGUGGUACAUUACCUGGUA | 13399 |
| 2596 | CCUGCCG GAC UGUGGCUG | 3994 | CAGCCACA AGAA GGCAGG ACCAGAGAAACACACGUUGUGGUACAUUACCUGGUA | 13400 |
| 2653 | AAGCUCU GAU GACCGAAC | 3995 | GUUCGGUC AGAA GAGCUU ACCAGAGAAACACACGUUGUGGUACAUUACCUGGUA | 13401 |
| 2743 | GGCCUCU GAU GGUGAUCG | 3996 | CGAUCACC AGAA GAGGCC ACCAGAGAAACACACGUUGUGGUACAUUACCUGGUA | 13402 |
| 2779 | GAAACCU GUC CAACUACC | 3997 | GGUAGUUG AGAA GGUUUC ACCAGAGAAACACACGUUGUGGUACAUUACCUGGUA | 13403 |
| 2814 | UUAUUCU GUC UCAACAAG | 3998 | CUUGUUGA AGAA GAAUAA ACCAGAGAAACACACGUUGUGGUACAUUACCUGGUA | 13404 |
| 2831 | GGACGCA GCC UUGCAUAU | 3999 | AUAUGCAA AGAA GCGUCC ACCAGAGAAACACACGUUGUGGUACAUUACCUGGUA | 13405 |
| 2895 | AAGCCCC GCC UAGACAGU | 4000 | ACUGUCUA AGAA GGGCUU ACCAGAGAAACACACGUUGUGGUACAUUACCUGGUA | 13406 |
| 2913 | GUCAGCA GCU CAAGUGUC | 4001 | GACACUUG AGAA GCUGAC ACCAGAGAAACACACGUUGUGGUACAUUACCUGGUA | 13407 |
| 2928 | GUCACCA GCU CCAGCUUC | 4002 | GAAGCUGG AGAA GGUGAC ACCAGAGAAACACACGUUGUGGUACAUUACCUGGUA | 13408 |
| 2934 | AGCUCCA GCU UCCCUGAA | 4003 | UUCAGGGA AGAA GGAGCU ACCAGAGAAACACACGUUGUGGUACAUUACCUGGUA | 13409 |
| 3001 | CCAAGCA GCC CCUCACCA | 4004 | UGGUGAGG AGAA GCUUGG ACCAGAGAAACACACGUUGUGGUACAUUACCUGGUA | 13410 |
| 3022 | AAGACCU GAU UUCCUACA | 4005 | UGUAGGAA AGAA GGUCUU ACCAGAGAAACACACGUUGUGGUACAUUACCUGGUA | 13411 |
| 3033 | UCCUACA GUU UCCAAGUG | 4006 | CACUUGGA AGAA GUAGGA ACCAGAGAAACACACGUUGUGGUACAUUACCUGGUA | 13412 |
| 3064 | AGUUUCU GUC CUCCAGAA | 4007 | UUCUGGAG AGAA GAAACU ACCAGAGAAACACACGUUGUGGUACAUUACCUGGUA | 13413 |
| 3179 | GAACCCU GAU UAUGUGAG | 4008 | CUCACAUA AGAA GGGUUC ACCAGAGAAACACACGUUGUGGUACAUUACCUGGUA | 10876 |
| 3357 | UUCUGCA GCC GCCUGAAG | 4009 | CUUCAGGC AGAA GCAGAA ACCAGAGAAACACACGUUGUGGUACAUUACCUGGUA | 13414 |
| 3360 | UGCAGCC GCC UGAAGGAA | 4010 | UUCCUUCA AGAA GCUGCA ACCAGAGAAACACACGUUGUGGUACAUUACCUGGUA | 13415 |
| 3379 | GCAUGCG GAU GAGAACCC | 4011 | GGGUUCUC AGAA GCAUGC ACCAGAGAAACACACGUUGUGGUACAUUACCUGGUA | 13416 |
| 3463 | GGCCCCG GUU UGCUGAAC | 4012 | GUUCAGCA AGAA GGGGCC ACCAGAGAAACACACGUUGUGGUACAUUACCUGGUA | 13417 |
| 3496 | GUGACCU GCU UCAAGCCA | 4013 | UGGCUUGA AGAA GGUCAC ACCAGAGAAACACACGUUGUGGUACAUUACCUGGUA | 13418 |
| 3553 | CCAUACU GAC UAGAAACA | 4014 | UGUUUCUA AGAA GUAUGG ACCAGAGAAACACACGUUGUGGUACAUUACCUGGUA | 13419 |
| 3615 | AAGGACG GCU UUGCAGAU | 4015 | AUCUGCAA AGAA GUCCUU ACCAGAGAAACACACGUUGUGGUACAUUACCUGGUA | 13420 |
| 3623 | CUUUGCA GAU CCACAUUU | 4016 | AAAUGUGG AGAA GCAAAG ACCAGAGAAACACACGUuGUGGUACAUUACCUGGUA | 13421 |
| 3650 | AAGCUCU GAU GAUGUGAG | 4017 | CUCACAUC AGAA GAGCUU ACCAGAGAAACACACGUUGUGGUACAUUACCUGGUA | 13422 |
| 3754 | ACUAUCA GCU GGACACUA | 4018 | UAGUGUCC AGAA GAUAGU ACCAGAGAAACACACGUUGUGGUACAUUACCUGGUA | 13423 |
| 3772 | GCACUCU GCU GGGCUCCC | 4019 | GGGAGCCC AGAA GAGUGC ACCAGAGAAACACACGUUGUGGUACAUUACCUGGUA | 13424 |
| 3796 | UGAAGCG GUU CACCUGGA | 4020 | UCCAGGUG AGAA GCUUCA ACCAGAGAAACACACGUUGUGGUACAUUACCUGGUA | 13425 |
| 3881 | ACUUUCC GAU CUGCCGAG | 4021 | CUCGGCAG AGAA GAAAGU ACCAGAGAAACACACGUUGUGGUACAUUACCUGGUA | 13426 |
| 3886 | CCGAUCU GCC GAGGGCCA | 4022 | UGGGCCUC AGAA GAUCGG ACCAGAGAAACACACGUUGUGGUACAUUACCUGGUA | 13427 |
| 3897 | AGGCCCA GCU UCUGCUUC | 4023 | GAAGCAGA AGAA GGGCCU ACCAGAGAAACACACGUUGUGGUACAUUACCUGGUA | 13428 |
| 3903 | AGCUUCU GCU UCUCCAGC | 4024 | GCUGGAGA AGAA GAAGCU ACCAGAGAAACACACGUUGUGGUACAUUACCUGGUA | 13429 |
| 3912 | UUCUCCA GCU GUGGCCAC | 4026 | GUGGCCAC AGAA GGAGAA ACCAGAGAAACACACGUUGUGGUACAUUACCUGGUA | 13430 |
| 3969 | GAGUCCU GCU GUUCUCCA | 4026 | UGGAGAAC AGAA UGACUC ACCAGAGAAACACACGUUGUGGUACAUUACCUGGUA | 13431 |
| 3972 | UCCUGCU GUU CUCCACCC | 4027 | GGGUGGAG AGAA GCAGGA ACCAGAGAAACACACGUUGUGGUACAUUACCUGGUA | 13432 |
| 3986 | ACCCCCA GAC UACAACUC | 4028 | GAGUUGUA AGAA GGGGGU ACCAGAGAAACACACGUUGUGGUACAUUACCUGGUA | 13433 |
| 4018 | CCUCCCC GCC CGCCUAAA | 4029 | UUUAGGCG AGAA GGGAGG ACCAGAGAAACACACGUUGUGGUACAUUACCUGGUA | 13434 |
| 4022 | CCCGCCC GCC UAAAGCUU | 4030 | AAGCUUUA AGAA GGCGGG ACCAGAGAAACACACGUUGUGGUACAUUACCUGGUA | 13435 |
| 4040 | CUCACCA GCC CCGACAAC | 4031 | GUUGUCGG AGAA GGUGAG ACCAGAGAAACACACGUUGUGGUACAUUACCUGGUA | 13436 |
| 4053 | ACAACCA GCC CCUGACAG | 4032 | CUGUCAGG AGAA GGUUGU ACCAGAGAAACACACGUUGUGGUACAUUACCUGGUA | 13437 |
| 4095 | CUAUUCC GCU CCACAGGA | 4033 | UCCUGUGG AGAA GAAUAG ACCAGAGAAACACACGUUGUGGUACAUUACCUGGUA | 13438 |
| 4110 | GGAGCCA GCU GCUUUUCG | 4034 | CGAAAAGC AGAA GGCUCC ACCAGAGAAACACACGUUGUGGUACAUUACCUGGUA | 13439 |
| 4113 | GCCAGCU GCU UUUCGUGA | 4035 | UCACGAAA AGAA GCUGGC ACCAGAGAAACACACGUUGUGGUACAUUACCUGGUA | 13440 |
| 4168 | UGUUGCU GUU UUGACUAA | 4036 | UUAGUCAA AGAA GCAACA ACCAGAGAAACACACGUUGUGGUACAUUACCUGGUA | 13441 |
| 4290 | GGCGACC GCC CGCCCACC | 4037 | GGUGGGCG AGAA GUCGCC ACCAGAGAAACACACGUUGUGGUACAUUACCUGGUA | 13442 |
| 4294 | ACCGCCC GCC CACCGGCC | 4038 | GGCCGGUG AGAA GGCGGU ACCAGAGAAACACACGUUGUGGUACAUUACCUGGUA | 13443 |
| 4329 | CCCUGCA GCU GUGGGACU | 4039 | AGUCCCAC AGAA GCAGGG ACCAGAGAAACACACGUUGUGGUACAUUACCUGGUA | 13444 |
| 4378 | AUGCACU GAC CUGCUCUG | 4040 | CAGAGCAG AGAA GUGCAU ACCAGAGAAACACACGUUGUGGUACAUUACCUGGUA | 13445 |
| 4383 | CUGACCU GCU CUGUCUCU | 4041 | AGAGACAG AGAA GGUCAG ACCAGAGAAACACACGUUGUGGUACAUUACCUGGUA | 13446 |
| 4388 | CUGCUCU GUC UCUCUUAU | 4042 | AUAAGAGA AGAA GAGCAG ACCAGAGAAACACACGUUGUGGUACAUUACCUGGUA | 13447 |
| 4457 | UGCGUCC GUC CUGUGGAG | 4043 | CUCCACAG AGAA GACGCA ACCAGAGAAACACACGUUGUGGUACAUUACCUGGUA | 13448 |
| 4525 | GGCCUCC GCU GUUUCGGG | 4044 | CCCGAAAC AGAA GAGGCC ACCAGAGAAACACACGUUGUGGUACAUUACCUGGUA | 13449 |
| 4528 | CUCCGCU GUU UCGGGCCC | 4045 | GGGCCCGA AGAA GCGGAG ACCAGAGAAACACACGUUGUGGUACAUUACCUGGUA | 13450 |
| 4643 | GUCUUCU GUG GUCUGUUU | 4046 | AAACAGAC AGAA GAAGAC ACCAGAGAAACACACGUUGUGGUACAUUACCUGGUA | 13451 |
| 4650 | GUUGUCU GUU UACCAUCC | 4047 | GGAUGGUA AGAA GACAAC ACCAGAGAAACACACGUUGUGGUACAUUACCUGGUA | 13452 |
| 4724 | AUCAUCA GUU CCUCUAGU | 4048 | ACUAGAGG AGAA GAUGAU ACCAGAGAAACACACGUUGUGGUACAUUACCUGGUA | 13453 |
| 4771 | CAGGCCU GAC CUUCGCAU | 4049 | AUGCGAAG AGAA GGCCUG ACCAGAGAAACACACGUUGUGGUACAUUACCUGGUA | 13454 |
| 4785 | GCAUACU GCU CACGGGGA | 4050 | UCCCCGUG AGAA GUAUGC ACCAGAGAAACACACGUUGUGGUACAUUACCUGGUA | 13455 |
| 4809 | UGGUCCA GUU UGGCCUAG | 4051 | CUAGGGCA AGAA GGACCA ACCAGAGAAACACACGUUGUGGUACAUUACCUGGUA | 13456 |
| 4834 | GCCUACU GAU GGGCUCAA | 4052 | UUGAGCCC AGAA GUAGGC ACCAGAGAAACACACGUUGUGGUACAUUACCUGGUA | 13457 |
| 4912 | UUAUCCU GUU UUAUAUAU | 4053 | AUAUAUAA AGAA GGAUAA ACCAGAGAAACACACGUUGUGGUACAUUACCUGGUA | 13458 |
| 5119 | CAAGGCA GUC UGAGAGGA | 4054 | UCCUCUCA AGAA GCCUUG ACCAGAGAAACACACGUUGUGGUACAUUACCUGGUA | 13459 |
| 5144 | AGUAUCA GCC CAUAUUUA | 4055 | UAAAUAUG AGAA GAUACU ACCAGAGAAACACACGUUGUGGUACAUUACCUGGUA | 13460 |
| 5287 | UUCAUCU GUU UCAUACCU | 4056 | AGGUAUGA AGAA GAUGAA ACCAGAGAAACACACGUUGUGGUACAUUACCUGGUA | 13461 |
| 5363 | GGUGCCC GCU CUUUGGGG | 4057 | CCCCAAAG AGAA GGCACC ACCAGAGAAACACACGUUGUGGUACAUUACCUGGUA | 13462 |
| 5462 | CACACCU CCC GGAGCCGG | 4058 | CCGGCUCC AGAA GGUGUG ACCAGAGAAACACACGUUGUGGUACAUUACCUGGUA | 13463 |
| 5478 | GGUCACA GCU GGGCAGAC | 4059 | GUCUGCCC AGAA GUGACC ACCAGAGAAACACACGUUGUGGUACAUUACCUGGUA | 13464 |
| 5486 | CUGGGCA GAC GAUGAAUA | 4060 | UAUUCAUC AGAA GCCCAG ACCAGAGAAACACACGUUGUGGUACAUUACCUGGUA | 13465 |
| 5500 | AAUAGCU GCU UUGGGAGA | 4061 | UCUCCCAA AGAA GCUAUU ACCAGAGAAACACACGUUGUGGUACAUUACCUGGUA | 13466 |
| 5539 | AUUCUCU GAC CGGGCCAG | 4062 | CUGGCCCG AGAA GAGAAU ACCAGAGAAACACACGUUGUGGUACAUUACCUGGUA | 13467 |
| 5564 | GGUACCU GCU CCCCUGUG | 4063 | CACAGGGG AGAA GGUACC ACCAGAGAAACACACGUUGUGGUACAUUACCUGGUA | 13468 |
| 5597 | GUUUUCU GUC UGAUGAGA | 4064 | UCUCAUCA AGAA GAAAAC ACCAGAGPAACACACGUUGUGGUACAUUACCUGGUA | 13469 |
| 5601 | UCUGUCU GAU GAGACUGG | 4065 | CCAGUCUC AGAA GACAGA ACCAGAGAAACACACGUUGUGGUACAUUACCUGGUA | 13470 |
| 5639 | UGAGACA GCC UGCAGCCC | 4066 | GGGCUGCA AGAA GUCUCA ACCAGAGAAACACACGUUGUGGUACAUUACCUGGUA | 13471 |
| 5646 | GCCUGCA CCC CACUGUGG | 4067 | CCACAGUG AGAA GCAGGC ACCAGAGAAACACACGUUGUGGUACAUUACCUGGUA | 13472 |
| 5781 | CUUGUCG GCU GCAGUGUG | 4068 | CACACUGC AGAA GACAAG ACCAGAGAAACACACGUUGUGGUACAUUACCUGGUA | 13473 |
| 5829 | AGAAACG GAU GAGAACAG | 4069 | CUGUUCUC AGAA GUUUCU ACCAGAGAAACACACGUUGUGGUACAUUACCUGGUA | 13474 |
| 5842 | AACAGCA GCC UGAGGUUU | 4070 | AAACCUCA AGAA GCUGUU ACCAGAGAAACACACGUUGUGGUACAUUACCUGGUA | 13475 |
| 5915 | UUAUUCC GAU UUUAAUAA | 4071 | UUAUUAAA AGAA GAAUAA ACCAGAGAAACACACGUUGUGGUACAUUACCUGGUA | 13476 |
| 6010 | UGGUUCU GCU UAAAGACU | 4072 | AGUCUUUA AGAA GAACCA ACCAGAGAAACACACGUUGUGGUACAUUACCUGGUA | 13477 |

TABLE X
Homologous Hammerhead Ribozyme Target Sites Between
Human flt-1 and KDR RNA

| nt. | fit-1 | Seq | nt. | KDR | Seq |
|---|---|---|---|---|---|
| Position | Target Sequence | ID No | Position | Target Sequence | ID No |
| 3388 | CCGGGAU A UUUAUAA | 4073 | 3151 | CCGGGAU A UUUAUAA | 4073 |
| 2174 | AAUGUAU A CACAGGG | 4074 | 3069 | AgUGUAU c CACAGUG | 4116 |
| 2990 | UGCAAAU A UGGAAAU | 4075 | 2756 | UGCAAAU u UGGAAAc | 4117 |
| 2693 | CUCCCUU A UGAUGCC | 4076 | 2459 | CUgCCUU A UGAUGCC | 4118 |
| 2981 | GUUGAAU A CUGCAAA | 4077 | 2747 | GUgGAAU u CUGCAAA | 4119 |
| 1359 | UAUGGUU A AAAGAUG | 4078 | 2097 | UgUGGUU u AAAGAUa | 4120 |
| 3390 | GGGAUAU U UAUAAGA | 4079 | 3153 | GGGAUAU U UAUAAag | 4121 |
| 3391 | GGAUAUU U AUAAGAA | 4080 | 3154 | GGAUAUU U AUAAagA | 4122 |
| 2925 | ACGUGGU U AACCUGC | 4081 | 2691 | AuGUGGU c AACCUuC | 4123 |
| 7140 | UAUUUCU A GUCAUGA | 4082 | 2340 | UAGUUCU u GUCAUCA | 4124 |
| 1785 | CAAUAAU A GAAGGAA | 4083 | 1515 | CuCUAAU u GAAGGAA | 4125 |
| 2731 | GAGACUU A AACUGGG | 4084 | 768 | uuGACUU c AACUGGG | 4126 |
| 3974 | GAUGACU A CCAGGGC | 4085 | 1466 | GAgGACU u CCAGGGa | 4127 |
| 6590 | UUAAUGU A GAAAGAA | 4086 | 2603 | aaAAUGU u GAAAGAA | 4128 |
| 6705 | GCCAUUU A UGACAAA | 4087 | 3227 | aCaAUUU u UGACAgA | 4129 |
| 974 | GUCAAAU U ACUUAGA | 4088 | 147 | uUCAAAU U ACUUgGA | 4130 |
| 1872 | AUAAAGU U GGGACUG | 4089 | 1602 | AcAAAGU c GGGAgaG | 4131 |
| 2333 | ACUUGGU U UAAAAAC | 4090 | 1088 | AaaUGGU a UAAAAAu | 4132 |
| 2775 | AAGUGGU U CAAGCAU | 4091 | 1745 | AcaUGGU a CAAGCuU | 4133 |
| 3533 | UUCUCCU U AGGUGGG | 4092 | 3296 | UUuUCCU U AGGUGcu | 4134 |
| 3534 | UCUCCUG A GGUGGGU | 4093 | 3297 | UuUCCUU A GGUGcuU | 4135 |
| 3625 | GUACUCU A CUCCUGA | 4094 | 4054 | GagCUCU c CUCCUGu | 4136 |
| 1814 | AGCACCU U GGUUGUG | 4095 | 1059 | AGuACCU U GGUUacc | 4137 |
| 2744 | GGCAAAU C ACUUGGA | 4096 | 147 | uuCAAAU u ACUUGcA | 4130 |
| 2783 | CAAGCAU C AGCAUUU | 4097 | 796 | gAAGCAU C AGCAUaa | 4138 |
| 3613 | GAGAGCU C CUGAGUA | 4098 | 2968 | GgaAGCU C CUGAagA | 4139 |
| 4052 | AAGGCCU C GCUCAAG | 4099 | 1923 | uGuGCCU u GCUCAAG | 4140 |
| 5305 | UCUCCAU A UCAAAAC | 4100 | 456 | ggUCCAU u UCAAAuC | 4141 |
| 7158 | AUGUAUU U UGUAUAC | 4101 | 631 | gUCUAUU a UGUAcAu | 4142 |
| 1836 | CUAGAAU U UCUGGAA | 4102 | 1007 | aUgGAAU c UCUGGug | 4143 |
| 2565 | CUCUCUG C UGGCUCC | 4103 | 2328 | uguUCUU C UGGCUaC | 4144 |
| 4250 | CUGUACU C CACCCCA | 4104 | 3388 | uUaUACU a CACCagA | 4145 |
| 7124 | ACAUGGU U UGGUCCU | 4105 | 3778 | cagUGGU a UGGUuCU | 4146 |
| 436 | AUGGUCU U UGCCUGA | 4106 | 1337 | AcGGUCU a UGCCauu | 4147 |
| 2234 | GCACCAG A CCUCCUG | 4107 | 1344 | augCCAU u CCUCCcc | 4148 |
| 2763 | GGGCUUU U GGAAAAG | 4108 | 990 | uuGCUUU U GGAAguG | 4149 |
| 4229 | CCAGACU A CAACUCG | 4109 | 767 | auuGACU u CAACUgG | 4150 |
| 5301 | GUUUUCU C CAGAUCA | 4110 | 3307 | ugcUUCU C CAUAUCc | 4151 |
| 6015 | AGAAUGU A UGCCUCU | 4111 | 1917 | ACuAUGU c UGCCUug | 4152 |
| 6095 | AUUCCCU A GUGAGCC | 4112 | 1438 | AUaCCCU u GUGAaga | 4153 |
| 6236 | UGUUGUU C CUCUUCU | 4113 | 76 | UagUGUU u CUCUUga | 4154 |
| 5962 | GCUUCCU U UUAUCCA | 4114 | 3099 | auaUCCU c UUAUCgg | 4155 |
| 7629 | UAUAUAU U CUCUGCU | 4115 | 3096 | gAaAUAU c CUCUuaU | 4156 |

Lowercase letters are used to represent sequence variance between flt-1 and KDR RNA

TABLE XI
A. 2.5 μmol Synthesis Cycle ABI 394 Instrument

| | | | | | | Wait Time* | | | |
|---|---|---|---|---|---|---|---|---|---|
| | | | | Wait Time* | | 2′- | | Wait Time* | |
| Reagent | Equivalents | Amount | | DNA | | O-methyl | | RNA | |
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl Imidazole | 186 | 233 | μL | 5 | sec | 5 | sec | 5 | sec |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | Wait Time* | | | |
|---|---|---|---|---|---|---|---|---|---|
| | | | | Wait Time* | | 2′- | | Wait Time* | |
| Reagent | Equivalents | Amount | | DNA | | O-methyl | | RNA | |
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl Imidazole | 1245 | 124 | μL | 5 | sec | 5 | sec | 5 | sec |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | | | | | | Wait Time* | | | |
|---|---|---|---|---|---|---|---|---|---|
| | Equivalents | Amount | | Wait Time* | | 2′- | | Wait Time* | |
| Reagent | DNA/2′-O-methyl/Ribo | DNA/2′-O-methyl/Ribo | | DNA | | O-methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl Imidazole | 502/502/502 | 50/50/50 | μL | 10 | sec | 10 | sec | 10 | sec |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE XII
Ribozyme and attenuated control sequences and locations of modified nucleotides.

| | RPI | | | Seq. |
|---|---|---|---|---|
| Target Site* | No. | Ribozyme Sequence^{†} (5′-3′) | Activity | ID No. |
| Flt-1358-amino | RPI 4118 | 5′ aₛuₛcₛuₛuuu cUGAUGaggcgaaagccGaa AccauacB 3′ | Active | 13478 |
| Flt-1358-amino (att.) | RPI 4581 | aₛuₛcₛuₛuuu cUuuAuGaggcgaaagccGau AccauacB | Attenuated | 13479 |
| Flt-1358-allyl | RPI 4159 | 5′ aₛuₛcₛuₛuuu cUGAuGaggcgaaagccGaa AccauacB 3′ | Active | 13480 |
| Flt-1358-allyl (att.) | RPI 4583 | aₛuₛcₛuₛuuu cUuAuGaggcgaaagccGau AccauacB | Attenuated | 13481 |
| Flt-4229-amino | RPT 4131 | gₛaₛgₛuₛug cUGAUGaggcgaaagccGaa AgucugB | Active | 13482 |
| Flt-4229-amino (att.) | RPI 4582 | gₛaₛgₛuₛug cUuAUGaggcgaaagccGau AgucugB | Attenuated | 13483 |
| Flt-4229-allyl | RPI 4172 | gₛaₛgₛuₛug cUGAuGaggcgaaagccGaa AgucugB | Active | 13484 |
| Flt-4229-allyl (att.) | RPI 4584 | gₛaₛgₛuₛug cUuAuGaggcgaaagccGau AgucugB | Attenuated | 13485 |
| KDR-726-amino | RPI 4097 | uₛaₛcₛaₛauu cUGAUGaggcgaaagccGaa AagacaaB | Active | 13486 |
| KDR-726-amino (att.) | RPI 4674 | uₛaₛcₛaₛauu cUuAUGaggcgaaagccGau AagacaaB | Attenuated | 13487 |
| KDR-726-allyl | RPI 4138 | uₛaₛcₛaₛauu cUGAuGaggcgaaagccGaa AagacaaB | Active | 13488 |
| KDR-726 allyl (att.) | | uₛaₛcₛaₛauu cUuAuGaggcgaaagccGau AagacaaB | Attenuated | 13489 |
| KDR-3950-amino | RPI 4110 | cₛuₛgₛgₛag cUGAUGaggcgaaagccGaa AcacggB | Active | 13490 |
| KDR-3950-amino (att.) | RPI 4677 | cₛuₛgₛgₛag cUuAuGaggcgaaagccGau AcacggB | Attenuated | 13491 |
| KDR-3950-allyl | RPI 4151 | cₛuₛgₛgₛag cUcAuGaggcgaaagccGaa AcacggB | Active | 13492 |
| KDR-3950-allyl (att.) | | cₛuₛgₛgₛag cUuAuGaggcgaaagccGau AcacggB | Attenuated | 13493 |
| | | binding arm/catalytic core & stem II/binding arm | | |

*Ribozyme target sites are named according to the cleavage position in human Flt-1 (EMBL accession no. X51602) or human KDR mRNA (EMBL accession no. L0497).
^{\}Modifications are indicated as follows:
2′-O-methyl nucleotides, lowercase;
ribonucleotides, uppercase G, A;
inverted 3′—3′ deoxyabasic, B.
Two ribozyme motifs were tested ²³.
For the NH₂-modified ribozymes, U indicates the two core positions where 2′-NH₂ uridines are present.
U indicates 2′-C-allyl-uridine modification.
The positions of four phosphorothioate linkages at the 5′ end are indicated by a subscript “s”.
Changes in the attenuated controls are underlined.
Nucleotides comprising the base paired region of stem II are in italics.
A 3 bp stem II is shown above.
Ribozymes or attenuated controls referred to in the text with a 4 bp stem II have one additional base pair such that the stem II/loop sequence is ggccgaaaggcc.

TABLE XIII
Ribozyme and attenuated control sequences
with locations of modified nucleotides.

| Ribozyme | Sequence^{\}(5′-3′) | Activity | Seq. ID Nos. |
|---|---|---|---|
| RPI.4610 | gₛaₛgₛuₛug cUGAuGaggccgaaaggccGaa AgucugB | Active | 13494 |
| (ANGIOZYME ™) | | | |
| RPI.13141 | gₛaₛgₛuₛug cUAGaGaggccgaaaggccGau AgucugB | BAC^{‡} | 13495 |
| RPI.13030 | gₛaₛaₛgₛgu cUAGuGaggccgaaaggccGau AugucB | SAC* | 13496 |
| | binding arm/core & stem II/binding arm | | |

^{\}Modifications are indicated as follows:
2′-O-methyl nucleotides, lowercase;
ribonucleotides, uppercase G, A;
2′-C-allyl uridine, U;
inverted 3′—3′ deoxyabasic, B.
The positions of four phosphorothioate linkages at the 5′ end are indicated by a subscript “s”.
Nucleotides comprising the base paired region of stem II are in italics.
Nucleotide changes in the core of the attenuated controls are underlined.
ANGIOZYME ribozyme is targeted to cleave site 4229 in human flt-1 RNA and is identical to Flt-4229-allyl ribozyme (Table XII) except that the ANGIOZYME has a stem II of length 4 bps.
^{‡}BAC: Binding arm, Attenuated core Control.
*SAC: Scrambled arm, Attenuated core Control.

TABLE XIV
NCH Ribozyme and Target Sequences

| | | Seq ID | | Seq ID |
|---|---|---|---|---|
| Pos | Target | No. | NCH Ribozyme | No. |
| 9 | GCGGACAC U CGUCUCGG | 4157 | CCGAGAGG CUGAUGAGGCCGUUAGGCCGAA IUGUCCGC | 13497 |
| 11 | GGACACUC C UCUCGGCU | 4158 | AGCCGAGA CUGAUGAGGCCGUUAGGCCGAA IAGUGUCC | 13498 |
| 12 | GACACUCC U CUCGGCUC | 4159 | GAGCCGAG CUGAUGAGGCCGUUAGGCCGAA IGAGUGUC | 13499 |
| 14 | CACUCCUC U CGGCUCCU | 4160 | AGGAGCCG CUGAUGAGGCCGUUAGGCCGAA IAGGAGUG | 13500 |
| 19 | CUCUCGGC U CCUCCCCG | 4161 | CGGGGAGG CUGAUGAGGCCGUUAGGCCGAA ICCGAGAG | 13501 |
| 21 | CUCGGCUC C UCCCCGGC | 4162 | GCCGGGGA CUGAUGAGGCCGUUAGGCCGAA IAGCCGAG | 13502 |
| 22 | UCGGCUCC U CCCCGGCA | 4163 | UGCCGGGG CUGAUGAGGCCGUUAGGCCGAA IGAGCCGA | 13503 |
| 24 | GGCUCCUC C CCGGCAGC | 4164 | GCUGCCGG CUGAUGAGGCCGUUAGGCCGAA IAGGAGCC | 13504 |
| 25 | UCUCCUCG C CGGCAGCG | 4165 | CGCUGCCG CUGAUGAGGCCGUUAGGCCGAA IGAGGAGC | 13505 |
| 26 | CUCCUCCC C GGCAGCGG | 4166 | CCGCUGCC CUGAUGAGGCCGUUAGGCCGAA IGGAGGAG | 13506 |
| 30 | UCCCCGGC A GCGGCGGC | 4167 | GCCGCCGC CUGAUGAGGCCGUUAGGCCGAA ICCGGGGA | 13507 |
| 42 | GCGGCGGC U CGGAGCGG | 4168 | CCGCUCCG CUGAUGAGGCCGUUAGGCCGAA ICCGCCGC | 13508 |
| 53 | GAGCGGGC U CCGGGGCU | 4169 | AGCCCCGG CUGAUGAGGCCGUUAGGCCGAA ICCCGCUC | 13509 |
| 55 | GCGGGCUC C GGGGCUCG | 4170 | CGAGCCCC CUGAUGAGGCCGUUAGGCCGAA IAGCCCGC | 13510 |
| 61 | UCCGGGGC U CGGGUGCA | 4171 | UGCACCCG CUGAUGAGGCCGUUAGGCCGAA ICCCCGGA | 13511 |
| 69 | UCGGGUGC A GCGGCCAG | 4172 | CUGGCCGC CUGAUGAGGCCGUUAGGCCGAA ICACCCGA | 13512 |
| 75 | GCAGCGGC C AGCGGGCC | 4173 | GGCCCGCU CUGAUGAGGCCGUUAGGCCGAA ICCGCUGC | 13513 |
| 76 | CAGCGGCC A GCGGGCCU | 4174 | AGGCCCGC CUGAUGAGGCCGUUAGGCCGAA IGCCGCUG | 13514 |
| 83 | CAGCGGGC C UGGCGGCG | 4175 | CGCCGCCA CUGAUGAGGCCGUUAGGCCGAA ICCCGCUG | 13515 |
| 84 | AGCGGGCC U GGCGGCGA | 4176 | UCGCCGCC CUGAUGAGGCCGUUAGGCCGAA IGCCCGCU | 13516 |
| 100 | AGGAUUAC C CGGGGAAG | 4177 | CUUCCCCG CUGAUGAGGCCGUUAGGCCGAA IUAAUCCU | 13517 |
| 101 | GGAUUACC C GGGGAAGU | 4178 | ACUUCCCC CUGAUGAGGCCGUUAGGCCGAA IGUAAUCC | 13518 |
| 117 | UGGUUGUC U CCUGGCUG | 4179 | CAGCCAGG CUGAUGAGGCCGUUAGGCCGAA IACAACCA | 13519 |
| 119 | GUUGUCUC C UGGCUGGA | 4180 | UCCAGCCA CUGAUGAGGCCGUUAGGCCGAA IAGACAAC | 13520 |
| 120 | UUGUCUCC U GGCUGGAG | 4181 | CUCCAGCC CUGAUGAGGCCGUUAGGCCGAA IGAGACAA | 13521 |
| 124 | CUCCUGGC U GGAGCCGC | 4182 | GCGGCUCC CUGAUGAGGCCGUUAGGCCGAA ICCAGGAG | 13522 |
| 130 | GCUGGAGC C GCGAGACG | 4183 | CGUCUCGC CUGAUGAGGCCGUUAGGCCGAA ICUCCAGC | 13523 |
| 144 | ACGGGCGC U CAGGGCGC | 4184 | GCGCCCUG CUGAUGAGGCCGUUAGGCCGAA ICGCCCGU | 13524 |
| 146 | GGGCGCUC A GGGCGCGG | 4185 | CCGCGCCC CUGAUGAGGCCGUUAGGCCGAA IAGCGCCC | 13525 |
| 158 | CGCGGGGC C GGCGGCGG | 4186 | CCGCCGCC CUGAUGAGGCCGUUAGGCCGAA ICCCCGCG | 13526 |
| 184 | GGACGGAC U CUGGCGGC | 4187 | GCCGCCAG CUGAUGAGGCCGUUAGGCCGAA IUCCGUCC | 13527 |
| 186 | ACGGACUC U GGCGGCCG | 4188 | CGGCCGCC CUGAUGAGGCCGUUAGGCCGAA IAGUCCGU | 13528 |
| 193 | CUGGCGGC C GGGUCGUU | 4189 | AACGACCC CUGAUGAGGCCGUUAGGCCGAA ICCGCCAG | 13529 |
| 205 | UCGUUGGC C GGGGGAGC | 4190 | GCUCCCCC CUGAUGAGGCCGUUAGGCCGAA ICCAACGA | 13530 |
| 220 | GCGCGGGC A CCGGGCGA | 4191 | UCGCCCGG CUGAUGAGGCCGUUAGGCCGAA ICCCGCGC | 13531 |
| 222 | GCGGGCAC C GGGCGAGC | 4192 | GCUCGCCC CUGAUGAGGCCGUUAGGCCGAA IUGCCCGC | 13532 |
| 231 | GGGCGAGC A GGCCGCGU | 4193 | ACGCGGCC CUGAUGAGGCCGUUAGGCCGAA ICUCGCCC | 13533 |
| 235 | GAGCAGGC C GCGUCGCG | 4194 | CGCGACGC CUGAUGAGGCCGUUAGGCCGAA ICCUCCUC | 13534 |
| 245 | CGUCGCGC U CACCAUGG | 4195 | CCAUGGUG CUGAUGAGGCCGUUAGGCCGAA ICGCGACG | 13535 |
| 247 | UCGCGCUC A CCAUGGUC | 4196 | GACCAUGG CUGAUGAGGCCGUUAGGCCGAA IAGCGCGA | 13536 |
| 249 | GCGCUCAC C AUGGUCAG | 4197 | CUGACCAU CUGAUGAGGCCGUUAGGCCGAA IUGAGCGC | 13537 |
| 250 | CGCUCACC A UGGUCAGC | 4198 | GCUGACCA CUGAUGAGGCCGUUAGGCCGAA IGUGAGCG | 13538 |
| 256 | CCAUGGUC A GCUACUGG | 4199 | CCAGUAGC CUGAUGAGGCCGUUAGGCCGAA IACCAUGG | 13539 |
| 259 | UGGUCAGC U ACUGGGAC | 4200 | GUCCCAGU CUGAUGAGGCCGUUAGGCCGAA ICUGACCA | 13540 |
| 262 | UCACCUAC U GGGACACC | 4201 | GGUGUCCC CUGAUGAGGCCGUUAGGCCGAA IUAGCUGA | 13541 |
| 268 | ACUGGGAC A CCGGGGUC | 4202 | GACCCCGG CUGAUGAGGCCGUUAGGCCGAA IUCCCAGU | 13542 |
| 270 | UGGGACAC C CCGGUCCU | 4203 | AGGACCCC CUGAUGAGGCCGUUAGGCCGAA IUGUCCCA | 13543 |
| 277 | CCGGGGUC C UGCUGUGC | 4204 | GCACAGCA CUGAUGAGGCCGUUAGGCCGAA IACCCCGG | 13544 |
| 278 | CQGGGUCC U GCUGUGCG | 4205 | CCCACAGC CUGAUGAGGCCGUUAGGCCGAA IGACCCCG | 13545 |
| 281 | GGUCCUGC U GUGCCCGC | 4206 | GCCCGCAC CUGAUGAGGCCGUUAGGCCGAA ICAGGACC | 13546 |
| 290 | CUGCGCCC U GCUCAGCU | 4207 | AUCUGAUC CUGAUGAGGCCGUUAGGCCGAA ICGCGCAC | 13547 |
| 293 | CGCGCUGC U CAGCUGUC | 4208 | GACAGCUG CUGAUGAGGCCGUUAGGCCGAA ICAGCGCG | 13548 |
| 295 | CGCUGCUC A GCUGUCUG | 4209 | CAGACAGC CUGAUGAGGCCGUUAGGCCGAA IAGCAGCG | 13549 |
| 298 | UGCUCAGC U GUCUGCUU | 4210 | AAGCAGAC CUGAUGAGGCCGUUAGGCCGAA ICUGAGCA | 13550 |
| 302 | CAGCUGUC U GCUUCUCA | 4211 | UGAGAAGC CUGAUGAGGCCGUUAGGCCGAA IACAGCUG | 13551 |
| 305 | CUGUCUGC U UCUCACAG | 4212 | CUGUGAGA CUGAUGAGGCCGUUAGGCCGAA ICAGACAG | 13552 |
| 308 | UCUGCUUC U CACAGGAU | 4213 | AUCCUGUG CUGAUGAGGCCGUUAGGCCGAA IAAGCAGA | 13553 |
| 310 | UGCUUCUC A CAGGAUCU | 4214 | AGAUCCUG CUGAUGAGGCCGUUAGGCCGAA IAGAAGCA | 13554 |
| 312 | CUUCUCAC A GGAUCUAG | 4215 | CUAGAUCC CUGAUGAGGCCGUUAGGCCGAA IUGAGAAG | 13555 |
| 318 | ACAGGAUC U AGUUCAGG | 4216 | CCUGAACU CUGAUGAGGCCGUUAGGCCGAA IAUCCUGU | 13556 |
| 324 | UCUAGUUC A GGUUCAAA | 4217 | UUUGAACC CUGAUGAGGCCGUUAGGCCGAA IAACUAGA | 13557 |
| 330 | UCAGGUUC A AAAUUAAA | 4218 | UUUAAUUU CUGAUGAGGCCGUUAGGCCGAA IAACCUGA | 13558 |
| 344 | AAAAGAUC C UGAACUGA | 4219 | UCAGUUCA CUGAUGAGGCCGUUAGGCCGAA IAUCUUUU | 13559 |
| 345 | AAAGAUCC U GAACUGAG | 4220 | CUCAGUUC CUGAUGAGGCCGUUAGGCCGAA IGAUCUUU | 13560 |
| 350 | UCCUGAAC U GAGUUUAA | 4221 | UUAAACUC CUGAUGAGGCCGUUAGGCCGAA IUUCAGGA | 13561 |
| 364 | UAAAAGGC A CCCAGCAC | 4222 | GUGCUGGG CUGAUGAGGCCGUUAGGCCGAA ICCUUUUA | 13562 |
| 366 | AAAGGCAC C CAGCACAU | 4223 | AUGUGCUG CUGAUGAGGCCGUUAGGCCGAA IUGCCUUU | 13563 |
| 367 | AAGGCACC C AGCACAUC | 4224 | GAUGUGCU CUGAUGAGGCCGUUAGGCCGAA IGUGCCUU | 13564 |
| 368 | AGGCACCC A GCACAUCA | 4225 | UGAUGUGC CUGAUGAGGCCGUUAGGCCGAA IGGUGCCU | 13565 |
| 371 | CACCCAGC A CAUCAUGC | 4226 | GCAUGAUG CUGAUGAGGCCGUUAGGCCGAA ICUGGGUG | 13566 |
| 373 | CCCAGCAC A UCAUGCAA | 4227 | UUGCAUGA CUGAUGAGGCCGUUAGGCCGAA IUGCUGGG | 13567 |
| 376 | AGCACAUC A UGCAAGCA | 4228 | UGCUUGCA CUGAUGAGGCCGUUAGGCCGAA IAUGUGCU | 13568 |
| 380 | CAUCAUGC A AGCAGGCC | 4229 | GGCCUGCU CUGAUGAGGCCGUUAGGCCGAA ICAUGAUG | 13569 |
| 384 | AUGCAAGC A GGCCAGAC | 4230 | GUCUGGCC CUGAUGAGGCCGUUAGGCCGAA ICUUGCAU | 13570 |
| 388 | AAGCAGGC C AGACACUG | 4231 | CAGUGUCH CUGAUGAGGCCGUUAGGCCGAA ICCUGCUU | 13571 |
| 389 | AGCAGGCC A GACACUGC | 4232 | GCAGUGUC CUGAUGAGGCCGUUAGGCCGAA IGCCUGCU | 13572 |
| 393 | GGCCAGAC A CUGCAUCU | 4233 | AGAUGCAG CUGAUGAGGCCGUUAGGCCGAA IUCUGGCC | 13573 |
| 395 | CCAGACAC U GCAUCUCC | 4234 | GGAGAUGC CUGAUGAGGCCGUUAGGCCGAA IUGUCUGG | 13574 |
| 398 | GACACUGC A UCUCCAAU | 4235 | AUUGGAGA CUGAUGAGGCCGUUAGGCCGAA ICAGUGUC | 13575 |
| 401 | ACUGCAUC U CCAAUGCA | 4236 | UGCAUUGG CUGAUGAGGCCGUUAGGCCGAA IAUGCAGU | 13576 |
| 403 | UGCAUCUC C AAUGCAGG | 4237 | CCUGCAUU CUGAUGAGGCCGUUAGGCCGAA IAGAUGCA | 13577 |
| 404 | GCAUCUCC A AUGCAGGG | 4238 | CCCUGCAU CUGAUGAGGCCGUUAGGCCGAA IGAGAUGC | 13578 |
| 409 | UCCAAUGC A GGGGGGAA | 4239 | UUCCCCCC CUGAUGAGGCCGUUAGGCCGAA ICAUUGGA | 13579 |
| 420 | GGGGAAGC A GCCCAUAA | 4240 | UUAUGGGC CUGAUGAGGCCGUUAGGCCGAA ICUUCCCC | 13580 |
| 423 | GAAGCAGC C CAUAAAUG | 4241 | CAUUUAUG CUGAUGAGGCCGUUAGGCCGAA ICUGCUUC | 13581 |
| 424 | AAGCAGCC C AUAAAUGG | 4242 | CCAUUUAU CUGAUGAGGCCGUUAGGCCGAA IGCUGCUU | 13582 |
| 425 | AGCAGCCC A UAAAUGGU | 4243 | ACCAUUUA CUGAUGAGGCCGUUAGGCCGAA IGGCUGCU | 13583 |
| 435 | AAAUGGUC U UUGCCUGA | 4244 | UCAGGCAA CUGAUGAGGCCGUUAGGCCGAA IACCAUUU | 13584 |
| 440 | GUCUUUGC C UGAAAUGG | 4245 | CCAUUUCA CUGAUGAGGCCGUUAGGCCGAA ICAAAGAC | 13585 |
| 441 | UCUUUGCC U GAAAUGGU | 4246 | ACCAUUUC CUGAUGAGGCCGUUAGGCCGAA IGCAAAGA | 13586 |
| 470 | CGAAAGGC U GAGCAUAA | 4247 | UUAUGCUC CUGAUGAGGCCGUUAGGCCGAA ICCUUUCG | 13587 |
| 475 | GGCUGAGC A UAACUAAA | 4248 | UUUAGUUA CUGAUGAGGCCGUUAGGCCGAA ICUCAGCC | 13588 |
| 480 | AGCAUAAC U AAAUCUGC | 4249 | GCAGAUUU CUGAUGAGGCCGUUAGGCCGAA IUUAUGCU | 13589 |
| 486 | ACUAAAUC U GCCUGUGG | 4250 | CCACAGGC CUGAUGAGGCCGUUAGGCCGAA IAUUUAGU | 13590 |
| 489 | AAAUCUGC C UGUGGAAG | 4251 | CUUCCACA CUGAUGAGGCCGUUAGGCCGAA ICAGAUUU | 13591 |
| 490 | AAUCUGCC U GUGGAAGA | 4252 | UCUUCCAC CUGAUGAGGCCGUUAGGCCGAA IGCAGAUU | 13592 |
| 505 | GAAAUGGC A AACAAUUC | 4253 | GAAUUGUU CUGAUGAGGCCGUUAGGCCGAA ICCAUUUC | 13593 |
| 509 | UGGCAAAC A AUUCUGCA | 4254 | UGCAGAAU CUGAUGAGGCCGUUAGGCCGAA IUUUGGCA | 13594 |
| 514 | AACAAUUC U GCAGUACU | 4255 | AGUACUGC CUGAUGAGGCCGUUAGGCCGAA IAAUUGUU | 13595 |
| 517 | AAUUCUGC A GUACUUUA | 4256 | UAAAGUAC CUGAUGAGGCCGUUAGGCCGAA ICAGAAUU | 13596 |
| 522 | UGCAGUAC U UUAACCUU | 4257 | AAGGUUAA CUGAUGAGGCCGUUAGGCCGAA IUACUGCA | 13597 |
| 528 | ACUUUAAC C UUGAACAC | 4258 | GUGUUCAA CUGAUGAGGCCGUUAGGCCGAA IUUAAAGU | 13598 |
| 529 | CUUUAACC U UGAACACA | 4259 | UGUGGUCA CUGAUGAGGCCGUUAGGCCGAA IGUUAAAG | 13599 |
| 535 | CCUUGAAC A CAGCUCAA | 4260 | UUGAGCUG CUGAUGAGGCCGUUAGGCCGAA IUUCAAGG | 13600 |
| 537 | UUGAACAC A GCUCAAGC | 4261 | GCUUGAGC CUGAUGAGGCCGUUAGGCCGAA IUGUUCAA | 13601 |
| 540 | AACACAGC U CAAGCAAA | 4262 | UUUGCUUG CUGAUGAGGCCGUUAGGCCGAA ICUGUGUU 13602 | |
| 542 | CACACCUC A AGCAAACC | 4263 | GGUUUGCU CUGAUGAGCCGUUAGGCCCGAA IAGCUGUG | 13603 |
| 546 | GCUCAAGC A AACCACAC | 4264 | GUGUGGUU CUGAUGAGGCCGUUAGGCCGAA ICUUGAGC | 13604 |
| 550 | AAGCAAAC C ACACUGGC | 4265 | GCCAGUGU CUGAUGAGGCCGUUAGGCCGAA IUUUGCUU | 13605 |
| 551 | AGCAAACC A CACUGGCU | 4266 | AUCCAGUG CUGAUGAGGCCGUUAGGCCGAA IGUUUGCU | 13606 |
| 553 | CAAACCAC A CUGGCUUC | 4267 | GAAGCCAG CUGAUGAGGCCGUUAGGCCGAA IUGGUUUG | 13607 |
| 555 | AACCACAC U GGCUUCUA | 4268 | UAGAAGCC CUGAUGAGGCCGUUAGGCCGAA IUGUGGUU | 13608 |
| 559 | ACACUGGC U UCUACAGC | 4269 | GCUGUAGA CUGAUGAGGCCGUUAGGCCGAA ICCAGUGU | 13609 |
| 562 | CUGGCUUC U ACAGCUGC | 4270 | GCAGCUGU CUGAUGAGGCCGUUAGGCCGAA IAAGCCAG | 13610 |
| 565 | GCUUCUAC A GCUGCAAA | 4271 | UUUGCAGC CUGAUGAGGCCGUUAGGCCGAA IUAGAAGC | 13611 |
| 568 | UCUACAGC U GCAAAUAU | 4272 | AUAUUUGC CUGAUGAGGCCGUUAGGCCGAA ICUGUAGA | 13612 |
| 571 | ACAUCUGC A AAUAUCUA | 4273 | UAGAUAUU CUGAUGAGGCCGUUAGGCCGAA ICAGCUGU | 13613 |
| 578 | CAAAUAUC U AGCUGUAC | 4274 | GUACAGCU CUGAUGAGGCCGUUAGGCCGAA IAUAUUUG | 13614 |
| 582 | UAUCUAGC U GUACCUAC | 4275 | GUAGGUAC CUGAUGAGGCCGUUAGGCCGAA ICUAGAUA | 13615 |
| 587 | AGCUGUAC C UACUUCAA | 4276 | UUGAAGUA CUGAUGAGGCCGUUAGGCCGAA IUACAGCU | 13616 |
| 588 | GCUGUACC U ACUUCAAA | 4277 | UUUGAAGU CUGAUGAGGCCGUUAGGCCGAA IGUACAUC | 13617 |
| 591 | GUACCUAC U UCAAAGAA | 4278 | UUCUUUGA CUGAUGAGGCCGUUAGGCCGAA IUAGGUAC | 13618 |
| 594 | CCUACUUC A AAGAAGAA | 4279 | UUCUUCUU CUGAUGAGGCCGUUAGGCCGAA IAAGUAGG | 13619 |
| 609 | AAGGAAAC A GAAUCUGC | 4280 | GCAGAUUC CUGAUGAGGCCGUUAUGCCGAA IUUUCCUU | 13620 |
| 615 | ACAGAAUC U GCAAUCUA | 4281 | UAUAUUGC CUGAUGAGGCCGUUAGGCCGAA IAUUCUGU | 13621 |
| 618 | GAAUCUGC A AUCUAUAU | 4282 | AUAUAGAU CUGAUGAGGCCGUUAGGCCGAA ICAGADUC | 13622 |
| 622 | CUGCAAUC U AUAUAUUU | 4283 | AAAUAUAU CUGAUGAGGCCGUUAGGCCGAA IAUUGCAG | 13623 |
| 642 | AGUGAUAC A GGUAGACC | 4284 | GGUCUACC CUGAUGAGGCCGUUAGGCCGAA IUAUCACU | 13624 |
| 650 | AGGUAGAC C UUUCGUAG | 4285 | CUACGAAA CUGAUGAGGCCGUUAGGCCGAA IUCUACCU | 13625 |
| 651 | GGUAGACC U UUCGUAGA | 4286 | UCUACGAA CUGAUGAGGCCGUUAGGCCGAA IGUCUACC | 13626 |
| 667 | AGAUGUAC A GUGAAAUC | 4287 | GAUUUCAC CUGAUGAGGCCGUUAGGCCGAA IUACAUCU | 13627 |
| 676 | GUGAAAUC C CCGAAAUU | 4288 | AAUUUCGG CUGAUGAGGCCGUUAGGCCGAA IAUUUCAC | 13628 |
| 677 | UGAAAUCC C CGAAAUUA | 4289 | UAAUUUCG CUGAUGAGGCCGUUAGGCCGAA IGAUUUCA | 13629 |
| 678 | GAAAUCCC C GAAAUUAU | 4290 | AUAAUUUC CUGAUGAGGCCGUUAGGCCGAA IGGAUUUC | 13630 |
| 689 | AAUUAUAC A CAUGACUG | 4291 | CAGUCAUG CUGAUGAGGCCGUUAGGCCGAA IUAUAAUU | 13631 |
| 691 | UUAUACAC A UGACUGAA | 4292 | UUCAGUCA CUGAUGAGGCCGUUAGGCCGAA IUGUAUAA | 13632 |
| 696 | CACAUGAC U GAAGGAAG | 4293 | CUUCCUUC CUGAUGAGGCCGUUAGGCCGAA IUCAUGUG | 13633 |
| 710 | AAGGGAGC U CGUCAUUC | 4294 | GAAUGACG CUGAUGAGGCCGUUAGGCCGAA ICUCCCUU | 13634 |
| 715 | AGCUCGUC A UUCCCUGC | 4295 | GCAGGGAA CUGAUGAGGCCGUUAGGCCGAA IACGAGCU | 13635 |
| 719 | CGUCAUUC C CUGCCGGG | 4296 | CCCGGCAG CUGAUGAGGCCGUUAGGCCGAA IAAUGACG | 13636 |
| 720 | GUCAUUCC C UGCCGGGU | 4297 | ACCCGGCA CUGAUGAGGCCGUUAGGCCGAA IGAAUGAC | 13637 |
| 721 | UCAUUCCC U GCCGGGUU | 4298 | AACCCGGC CUGAUGAGGCCGUUAGGCCGAA IGGAAUGA | 13638 |
| 724 | UUCCCUGC C GGGUUACG | 4299 | CGUAACCC CUGAUGAGGCCGUUAGGCCGAA ICAGGGAA | 13639 |
| 735 | GUUACGUC A CCUAACAU | 4300 | AUGUUAGG CUGAUGAGGCCGUUAGGCCGAA IACGUAAC | 13640 |
| 737 | UACGUCAC C UAACAUCA | 4301 | UGAUGUGA CUGAUGAGGCCGUUAGGCCGAA IUGACGUA | 13641 |
| 738 | ACGUCACC U AACAUCAC | 4302 | GUGAUGUU CUGAUGAGGCCGUUAGGCCGAA IGUGACGU | 13642 |
| 742 | CACCUAAC A UCACUGUU | 4303 | AACAGUGA CUGAUGAGGCCGUUAGGCCGAA IUUAGGUG | 13643 |
| 745 | CUAACAUC A CUGUUACU | 4304 | AGUAACAG CUGAUGAGGCCGUUAGGCCGAA IAUGUUAG | 13644 |
| 747 | AACAUCAC U GUUACUUU | 4305 | AAAGUAAC CUGAUGAGGCCGUUAGGCCGAA IUGAUGUU | 13645 |
| 753 | ACUGUUAC U UUAAAAAA | 4306 | UUUUUUAA CUGAUGAGGCCGUUAGGCCGAA IUAACAGU | 13646 |
| 767 | AAAGUUUC C ACUUGACA | 4307 | UGUCAAGU CUGAUGAGGCCGUUAGGCCGAA IAAACUUU | 13647 |
| 768 | AACUUUCC A CUUGACAC | 4308 | GUGUCAAG CUGAUGAGGCCGUUAGGCCGAA IGAAACUU | 13648 |
| 770 | GUUUCCAC U UGACACUU | 4309 | AAGUGUCA CUGAUGAGGCCGUUAGGCCGAA IUGGAAAC | 13649 |
| 775 | CACUUGAC A CUUUCAUC | 4310 | GAUCAAAG CUGAUGAGGCCGUUAGGCCGAA IUCAAGUG | 13650 |
| 777 | CUUGACAC U UUGAUCCC | 4311 | GGGAUCAA CUGAUGAGGCCGUUAGGCCGAA IUGUCAAG | 13651 |
| 784 | CUUUGAUC C CUGAUGGA | 4312 | UCCAUCAG CUGAUGAGGCCGUUAGGCCGAA IAUCAAAG | 13652 |
| 785 | UUUGAUCC C UGAUGGAA | 4313 | UUCCAUCA CUGAUGAGGCCGUUAGGCCGAA IGAUCAAA | 13653 |
| 786 | UUGAUCCC U GAUGGAAA | 4314 | UUUCCAUC CUGAUGAGGCCGUUAGGCCGAA IGGAUCAA | 13654 |
| 799 | GAAAACGC A UAAUCUGG | 4315 | CCAGAUUA CUGAUGAGGCCGUUAGGCCGAA ICGUUUUC | 13655 |
| 805 | GCAUAAUC U GGGACAGU | 4316 | ACUGUCCC CUGAUGAGGCCGUUAGGCCGAA IAUUAUGC | 13656 |
| 811 | UCUGGGAC A GUAGAAAG | 4317 | CUUICUAC CUGAUGAGGCCGUUAGGCCGAA IUCCCAGA | 13657 |
| 823 | GAAAGGGC U UCAUCAUA | 4318 | UAUGAUGA CUGAUGAGGCCGUUAGGCCGAA ICCCUUUC | 13658 |
| 826 | AGGGCUUC A UCAUAUCA | 4319 | UGAUAUGA CUGAUGAGGCCGUUAGGCCGAA IAAGCCCU | 13659 |
| 829 | UCUUCAUC A UAUCAAAU | 4320 | AUUUGAUA CUGAUGAGGCCGUUAGGCCGAA IAUGAAGC | 13660 |
| 834 | AUCAUAUC A AAUGCAAC | 4321 | GUUGCAUU CUGAUGAGGCCGUUAGGCCGAA IAUAUGAU | 13661 |
| 840 | UCAAAUGC A ACGUACAA | 4322 | UUGUACGU CUGAUGAGGCCGUUAGGCCGAA ICAUUUGA | 13662 |
| 847 | CAACGUAC A AAGAAAUA | 4323 | UAUUUCUU CUGAUGAGGCCGUUAGGCCGAA IUACGUUG | 13663 |
| 860 | AAUAGGGC U UCUGACCU | 4324 | AGGUCAGA CUGAUGAGGCCGUUAGGCCGAA ICCCUAUU | 13664 |
| 863 | AGGGCUUC U GACCUGUG | 4325 | CACAGGUC CUGAUGAGGCCGUUAGGCCGAA IAAGCCCU | 13665 |
| 867 | CUUCUGAC C UGUGAAGC | 4326 | GCUUCACA CUGAUGAGGCCGUUAGGCCGAA IUCAGAAG | 13666 |
| 868 | UUCUGACC U GUGAAGCA | 4327 | UGCUUCAC CUGAUGAGGCCGUUAGGCCGAA IGUCAGAA | 13667 |
| 876 | UGUGAAGC A ACAGUCAA | 4328 | UUGACUGU CUGAUGAGGCCGUUAGGCCGAA ICUUCACA | 13668 |
| 879 | GAAGCAAC A GUCAAUGG | 4329 | CCAUUGAC CUGAUGAGGCCGUUAGGCCGAA IUUGCUUC | 13669 |
| 883 | CAACAGUC A AUGGGCAU | 4330 | AUGCCCAU CUGAUGAGGCCGUUAGGCCGAA IACUGUUG | 13670 |
| 890 | CAAUGGGC A UUUGUAUA | 4331 | UAUACAAA CUGAUGAGGCCGUUAGGCCGAA ICCCAUUG | 13671 |
| 903 | UAUAAGAC A AACUAUCU | 4332 | AGAUAGUU CUGAUGAGGCCGUUAGGCCGAA IUCUUAUA | 13672 |
| 907 | AGACAAAC U AUCUCACA | 4333 | UGUGAGAU CUGAUGAGGCCGUUAGGCCGAA IUUUGUCU | 13673 |
| 911 | AAACUAUC U CACACAUC | 4334 | GAUGUGUG CUGAUGAGGCCGUUAGGCCGAA IAUAGUUU | 13674 |
| 913 | ACUAUCUC A CACAUCGA | 4335 | UCGAUGUG CUGAUGAGGCCGUUAGGCCGAA IAGAUAGU | 13675 |
| 915 | UAUCUCAC A CAUCGACA | 4336 | UGUCGAUG CUGAUGAGGCCGUUAGGCCGAA IUGAGAUA | 13676 |
| 917 | UCUCACAC A UCGACAAA | 4337 | UUUGUCGA CUGAUGAGGCCGUUAGGCCGAA IUGUGAGA | 13677 |
| 923 | ACAUCGAC A AACCAAUA | 4338 | UAUUGGUU CUGAUGAGGCCGUUAGGCCGAA IUCGAUGU | 13678 |
| 927 | CGACAAAC C AAUACAAU | 4339 | AUUGUAUU CUGAUGAGGCCGUUAGGCCGAA IUUUGUCG | 13679 |
| 928 | GACAAACC A AUACAAUC | 4340 | GAUUGUAU CUGAUGAGGCCGUUAGGCCGAA IGUUUGUC | 13680 |
| 933 | ACCAAUAC A AUCAUAGA | 4341 | UCUAUGAU CUGAUGAGGCCGUUAGGCCGAA IUAUUGGU | 13681 |
| 937 | AUACAAUC A UAGAUGUC | 4342 | GACAUCUA CUGAUGAGGCCGUUAGGCCGAA IAUUGUAU | 13682 |
| 946 | UAGAUGUC C AAAUAAGC | 4343 | GCUUAUUU CUGAUGAGGCCGUUAGGCCGAA IACAUCUA | 13683 |
| 947 | AGAUGUCC A AAUAAGCA | 4344 | UGCUUAUU CUGAUGAGGCCGUUAGGCCGAA IGACAUCU | 13684 |
| 955 | AAAUAAGC A CACCACGC | 4345 | GCGUGGUG CUGAUGAGGCCGUUAGGCCGAA ICUUAUUU | 13685 |
| 957 | AUAAGCAC A CCACGCCC | 4346 | GGGCGUGG CUGAUGAGGCCGUUAGGCCGAA IUGCUUAU | 13686 |
| 959 | AAGCACAC C ACGCCCAG | 4347 | CUGGGCGU CUGAUGAGGCCGUUAGGCCGAA IUGUGCUU | 13687 |
| 960 | AGCACACC A CGCCCAGU | 4348 | ACUGGGCG CUGAUGAGGCCGUUAGGCCGAA IGUGUGCU | 13688 |
| 964 | CACCACGC C CAGUCAAA | 4349 | UUUGACUG CUGAUGAGGCCGUUAGGCCGAA ICGUGGUG | 13689 |
| 965 | ACCACGCC C AGUCAAAU | 4350 | AUUUGACU CUGAUGAGGCCGUUAGGCCGAA IGCGUGGU | 13690 |
| 966 | CCACGCCC A GUCAAAUU | 4351 | AAUUUGAC CUGAUGAGGCCGUUAGGCCGAA IGGCGUGG | 13691 |
| 970 | GCCCAGUC A AAUUACUU | 4352 | AAGUAAUU CUGAUGAGGCCGUUAGGCCGAA IACUGGGC | 13692 |
| 977 | CAAAUUAC U UAGAGGCC | 4353 | GGCCUCUA CUGAUGAGGCCGUUAGGCCGAA IUAAUUUG | 13693 |
| 985 | UUAGAGGC C AUACUCUU | 4354 | AAGAGUAU CUGAUGAGGCCGUUAGGCCGAA ICCUCUAA | 13694 |
| 986 | UAGAGGCC A UACUCUUG | 4355 | CAAGAGUA CUGAUGAGGCCGUUAGGCCGAA IGCCUCUA | 13695 |
| 990 | GGCCAUAC U CUUGUCCU | 4356 | AGGACAAG CUGAUGAGGCCGUUAGGCCGAA IUAUGGCC | 13696 |
| 992 | CCAUACUC U UGUCCUCA | 4357 | UGAGGACA CUGAUGAGGCCGUUAGGCCGAA IAGUAUGG | 13697 |
| 997 | CUCUUGUC C UCAAUUGU | 4358 | ACAAUUGA CUGAUGAGGCCGUUAGGCCGAA IACAAGAG | 13698 |
| 998 | UCUUGUCC U CAAUUGUA | 4359 | UACAAUUG CUGAUGAGGCCGUUAGGCCGAA IGACAAGA | 13699 |
| 1000 | UUGUCCUC A AUUGUACU | 4360 | AGUACAAU CUGAUGAGGCCGUUAGGCCGAA IAGGACAA | 13700 |
| 1008 | AAUUGUAC U GCUACCAC | 4361 | GUGGUAGC CUGAUGAGGCCGUUAGGCCGAA IUACAAUU | 13701 |
| 1011 | UGUACUGC U ACCACUCC | 4362 | GGAGUGGU CUGAUGAGGCCGUUAGGCCGAA ICAGUACA | 13702 |
| 1014 | ACUGCUAC C ACUCCCUU | 4363 | AAGGGAGU CUGAUGAGGCCGUUAGGCCGAA IUAGCAGU | 13703 |
| 1015 | CUGCUACC A CUCCCUUG | 4364 | CAAGGGAG CUGAUGAGGCCGUUAGGCCGAA IGUAGCAG | 13704 |
| 1017 | GCUACCAC U CCCUUGAA | 4365 | UUCAAGGG CUGAUGAGGCCGUUAGGCCGAA IUGGUAGC | 13705 |
| 1019 | UACCACUC C CUUGAACA | 4366 | UGUUCAAG CUGAUGAGGCCGUUAGGCCGAA IAGUGGUA | 13706 |
| 1020 | ACCACUCC C UUGAACAC | 4367 | GUGUUCAA CUGAUGAGGCCGUUAGGCCGAA IGAGUGGU | 13707 |
| 1021 | CCACUCCC U UGAACACG | 4368 | CGUGUUCA CUGAUGAGGCCGUUAGGCCGAA IGGAGUGG | 13708 |
| 1027 | CCUUGAAC A CGAGAGUU | 4369 | AACUCUCG CUGAUGAGGCCGUUAGGCCGAA IUUCAAGG | 13709 |
| 1037 | GAGAGUUC A AAUGACCU | 4370 | AGGUCAUU CUGAUGAGGCCGUUAGGCCGAA IAACUCUC | 13710 |
| 1044 | CAAAUGAC C UGGAGUUA | 4371 | UAACUCCA CUGAUGAGGCCGUUAGGCCGAA IUCAUUUG | 13711 |
| 1045 | AAAUGACC U GGAGUUAC | 4372 | GUAACUCC CUGAUGAGGCCGUUAGGCCGAA IGUCAUUU | 13712 |
| 1054 | GGAGUUAC C CUGAUGAA | 4373 | UUCAUCAG CUGAUGAGGCCGUUAGGCCGAA IUAACUCC | 13713 |
| 1055 | GAGUUACC C UGAUGAAA | 4374 | UUUCAUCA CUGAUGAGGCCGUUAGGCCGAA IGUAACUC | 13714 |
| 1056 | AGUUACCC U GAUGAAAA | 4375 | UUUUCAUC CUGAUGAGGCCGUUAGGCCGAA IGGUAACU | 13715 |
| 1077 | AAGAGAGC U UCCGUAAG | 4376 | CUUACGGA CUGAUGAGGCCGUUAGGCCGAA ICUCUCUU | 13716 |
| 1080 | AGAGCUUC C GUAAGGCG | 4377 | CGCCUUAC CUGAUGAGGCCGUUAGGCCGAA IAAGCUCU | 13717 |
| 1099 | GAAUUGAC C AAAGCAAU | 4378 | AUUGCUUU CUGAUGAGGCCGUUAGGCCGAA IUCAAUUC | 13718 |
| 1100 | AAUUGACC A AAGCAAUU | 4379 | AAUUGCUU CUGAUGAGGCCGUUAGGCCGAA IGUCAAUU | 13719 |
| 1105 | ACCAAAGC A AUUCCCAU | 4380 | AUGGGAAU CUGAUGAGGCCGUUAGGCCGAA ICUUUGGU | 13720 |
| 1110 | AGCAAUUC C CAUGCCAA | 4381 | UUGGCAUG CUGAUGAGGCCGUUAGGCCGAA IAAUUGCU | 13721 |
| 1111 | GCAAUUCC C AUGCCAAC | 4382 | GUUGGCAU CUGAUGAGGCCGUUAGGCCGAA IGAAUUGC | 13722 |
| 1112 | CAAUUCCC A UGCCAACA | 4383 | UGUUGGCA CUGAUGAGGCCGUUAGGCCGAA IGGAAUUG | 13723 |
| 1116 | UCCCAUGC C AACAUAUU | 4384 | AAUAUGUU CUGAUGAGGCCGUUAGGCCGAA ICAUGGGA | 13724 |
| 1117 | CCCAUGCC A ACAUAUUC | 4385 | GAAUAUGU CUGAUGAGGCCGUUAGGCCGAA IGCAUGGG | 13725 |
| 1120 | AUGCCAAC A UAUUCUAC | 4386 | GUAGAAUA CUGAUGAGGCCGUUAGGCCGAA IUUGGCAU | 13726 |
| 1126 | ACAUAUUC U ACAGUGUU | 4387 | AACACUGU CUGAUGAGGCCGUUAGGCCGAA IAAUAUGU | 13727 |
| 1129 | UAUUCUAC A GUGUUCUU | 4388 | AAGAACAC CUGAUGAGGCCGUUAGGCCGAA IUAGAAUA | 13728 |
| 1136 | CAGUGUUC U UACUAUUG | 4389 | CAAUAGUA CUGAUGAGGCCGUUAGGCCGAA IAACACUG | 13729 |
| 1140 | GUUCUUAC U AUUGACAA | 4390 | UUGUCAAU CUGAUGAGGCCGUUAGGCCGAA IUAAGAAC | 13730 |
| 1147 | CUAUUGAC A AAAUGCAG | 4391 | CUGCAUUU CUGAUGAGGCCGUUAGGCCGAA IUCAAUAG | 13731 |
| 1154 | CAAAAUGC A GAACAAAG | 4392 | CUUUGUUC CUGAUGAGGCCGUUAGGCCGAA ICAUUUUG | 13732 |
| 1159 | UGCAGAAC A AAGACAAA | 4393 | UUUGUCUU CUGAUGAGGCCGUUAGGCCGAA IUUCUGCA | 13733 |
| 1165 | ACAAAGAC A AAGGACUU | 4394 | AAGUCCUU CUGAUGAGGCCGUUAGGCCGAA IUCUUUGU | 13734 |
| 1172 | CAAAGGAC U UUAUACUU | 4395 | AAGUAUAA CUGAUGAGGCCGUUAGGCCGAA IUCCUUUG | 13735 |
| 1179 | CUUUAUAC U UGUCGUGU | 4396 | ACACGACA CUGAUGAGGCCGUUAGGCCGAA IUAUAAAG | 13736 |
| 1199 | GAGUGGAC C AUCAUUCA | 4397 | UGAAUGAU CUGAUGAGGCCGUUAGGCCGAA IUCCACUC | 13737 |
| 1200 | AGUGGACC A UCAUUCAA | 4398 | UUGAAUGA CUGAUGAGGCCGUUAGGCCGAA IGUCCACU | 13738 |
| 1203 | GGACCAUC A UUCAAAUC | 4399 | GAUUUGAA CUGAUGAGGCCGUUAGGCCGAA IAUGGUCC | 13739 |
| 1207 | CAUCAUUC A AAUCUGUU | 4400 | AACAGAUU CUGAUGAGGCCGUUAGGCCGAA IAAUGAUG | 13740 |
| 1212 | UUCAAAUC U GUUAACAC | 4401 | GUGUUAAC CUGAUGAGGCCGUUAGGCCGAA IAUUUGAA | 13741 |
| 1219 | CUGUUAAC A CCUCAGUG | 4402 | CACUGAGG CUGAUGAGGCCGUUAGGCCGAA IUUAACAG | 13742 |
| 1221 | GUUAACAC C UCAGUGCA | 4403 | UGCACUGA CUGAUGAGGCCGUUAGGCCGAA IUGUUAAC | 13743 |
| 1222 | UUAACACC U CAGUGCAU | 4404 | AUGCACUG CUGAUGAGGCCGUUAGGCCGAA IGUGUUAA | 13744 |
| 1224 | AACACCUC A GUGCAUAU | 4405 | AUAUGCAC CUGAUGAGGCCGUUAGGCCGAA IAGGUGUU | 13745 |
| 1229 | CUCAGUGC A UAUAUAUG | 4406 | CAUAUAUA CUGAUGAGGCCGUUAGGCCGAA ICACUGAG | 13746 |
| 1245 | GAUAAAGC A UUCAUCAC | 4407 | GUGAUGAA CUGAUGAGGCCGUUAGGCCGAA ICUUUAUC | 13747 |
| 1249 | AAGCAUUC A UCACUGUG | 4408 | CACAGUGA CUGAUGAGGCCGUUAGGCCGAA IAAUGCUU | 13748 |
| 1252 | CAUUCAUC A CUGUGAAA | 4409 | UUUCACAG CUGAUGAGGCCGUUAGGCCGAA IAUGAAUG | 13749 |
| 1254 | UUCAUCAC U GUGAAACA | 4410 | UGUGUCAC CUGAUGAGGCCGUUAGGCCGAA IUGAUGAA | 13750 |
| 1262 | UGUGAAAC A UCGAAAAC | 4411 | GUUUUCGA CUGAUGAGGCCGUUAGGCCGAA IUUUCACA | 13751 |
| 1271 | UCGAAAAC A GCAGGUGC | 4412 | GCACCUGC CUGAUGAGGCCGUUAGGCCGAA IUUUUCGA | 13752 |
| 1274 | AAAACAGC A UGUGCUUG | 4413 | CAAGCACC CUGAUGAGGCCGUUAGGCCGAA ICUGUUUU | 13753 |
| 1280 | GCAGGUGC U UGAAACCG | 4414 | CGGUUUCA CUGAUGAGGCCGUUAGGCCGAA ICACCUGC | 13754 |
| 1287 | CUUGAAAC C GUAGCUGG | 4415 | CCAGCUAC CUGAUGAGGCCGUUAGGCCGAA IUUUCAAG | 13755 |
| 1293 | ACCGUAGC U GGCAAGCG | 4416 | CGCUUGCC CUGAUGAGGCCGUUAGGCCGAA ICUACGGU | 13756 |
| 1297 | UAGCUGGC A AGCGGUCU | 4417 | AGACCUCU CUGAUGAGGCCGUUAGGCCGAA ICCAGCUA | 13757 |
| 1305 | AAGCGGUC U UACCGGCU | 4418 | AGCCGGUA CUGAUGAGGCCGUUAGGCCGAA IACCGCUU | 13758 |
| 1309 | GGUCUUAC C GGCUCUCU | 4419 | AGAGAGCC CUGAUGAGGCCGUUAGGCCGAA IUAAGACC | 13759 |
| 1313 | UUACCGGC U CUCUAUGA | 4420 | UCAUAGAG CUGAUGAGGCCGUUAGGCCGAA ICCGGUAA | 13760 |
| 1315 | ACCGGCUC U CUAUGAAA | 4421 | UUUCAUAG CUGAUGAGGCCGUUAGGCCGAA IAGCCGGU | 13761 |
| 1317 | CGGCUCUC U AUGAAAGU | 4422 | ACUUUCAU CUGAUGAGGCCGUUAGGCCGPA IAGAGCCG | 13762 |
| 1332 | GUGAAGGC A UUUCCCUC | 4423 | GAGGGAAA CUGAUGAGGCCGUUAGGCCGAA ICCUUCAC | 13763 |
| 1337 | GGCAUUUC C CUCGCCGG | 4424 | CCGGCGAG CUGAUGAGGCCGUUAGGCCGAA IAAAUGCC | 13764 |
| 1338 | GCAUUUCC C UCGCCGGA | 4425 | UCCGGCGA CUGAUGAGGCCGUUAGGCCGAA IGAAAUGC | 13765 |
| 1339 | CAUUUCCC U CGCCGGAA | 4426 | UUCCGGCG CUGAUGAGGCCGUUAGGCCGAA IGGAAAUG | 13766 |
| 1343 | UCCCUCGC C GGAAGUUG | 4427 | CAACUUCC CUGAUGAGGCCGUUAGGCCGAA ICGAGGGA | 13767 |
| 1373 | UGGGUUAC C UGCGACUG | 4428 | CAGUCGCA CUGAUGAGGCCGUUAGGCCGAA IUAACCCA | 13768 |
| 1374 | GGGUUACC U GCGACUGA | 4429 | UCAGUCGC CUGAUGAGGCCGUUAGGCCGAA IGUAACCC | 13769 |
| 1380 | CCUGCGAC U GAGAAAUC | 4430 | GAUUUCUC CUGAUGAGGCCGUUAGGCCGAA IUCGCAGG | 13770 |
| 1389 | GAGAAAUC U GCUCGCUA | 4431 | UAGCGAGC CUGAUGAGGCCGUUAGGCCGAA IAUUUCUC | 13771 |
| 1392 | AAAUCUGC U CGCUAUUU | 4432 | AAAUAGCG CUGAUGAGGCCGUUAGGCCGAA ICAGAUUU | 13772 |
| 1396 | CUGCUCGC U AUUUGACU | 4433 | AGUCAAAU CUGAUGAGGCCGUUAGGCCGAA ICGAGCAG | 13773 |
| 1404 | UAUUUGAC U CGUGGCUA | 4434 | UAGCCACG CUGAUGAGGCCGUUAGGCCGAA IUCAAAUA | 13774 |
| 1411 | CUCGUGGC U ACUCGUUA | 4435 | UAACGAGU CUGAUGAGGCCGUUAGGCCGAA ICCACGAG | 13775 |
| 1414 | GUGGCUAC U CGUUAAUU | 4436 | AAUUAACG CUGAUGAGGCCGUUAGGCCGAA IUAGCCAC | 13776 |
| 1426 | UAAUUAUC A AGGACGUA | 4437 | UACGUCCU CUGAUGAGGCCGUUAGGCCGAA IAUAAUUA | 13777 |
| 1437 | GACGUAAC U GAAGAGGA | 4438 | UCCUCUUC CUGAUGAGGCCGUUAGGCCGAA IUUACGUC | 13778 |
| 1449 | GAGGAUGC A GGGAAUUA | 4439 | UAAUUCCC CUGAUGAGGCCGUUAGGCCGAA ICAUCCUC | 13779 |
| 1461 | AAUUAUAC A AUCUUGCU | 4440 | AGCAAGAU CUGAUGAGGCCGUUAGGCCGAA IUAUAAUU | 13780 |
| 1465 | AUACAAUC U UGCUGAGC | 4441 | GCUCAGCA CUGAUGAGGCCGUUAGGCCGAA IAUUGUAU | 13781 |
| 1469 | AAUCUUGC U GAGCAUAA | 4442 | UUAUGCUC CUGAUGAGGCCGUUAGGCCGAA ICAAGAUU | 13782 |
| 1474 | UGCUGAGC A UAAkACAG | 4443 | CUGUUUUA CUGAUGAGGCCGUUAGGCCGAA ICUCACCA | 13783 |
| 1481 | CAUAAAAC A GUCAAAUG | 4444 | CAUUUGAC CUGAUGAGGCCGUUAGGCCGAA IUUUUAUG | 13784 |
| 1485 | AAACAGUC A AAUGUGUU | 4445 | AACACAUU CUGAUGAGGCCGUUAGGCCGAA IACUGUUU | 13785 |
| 1501 | UUAAAAAC C UCACUGCC | 4446 | GGCAGUGA CUGAUGAGGCCGUUAGGCCGAA IUUUUUAA | 13786 |
| 1502 | UAAAAACC U CACUGCCA | 4447 | UGGCAGUG CUGAUGAGGCCGUUAGGCCGAA IGUUUUUA | 13787 |
| 1504 | AAAACCUC A CUGCCACU | 4448 | AGUGUCAG CUGAUGAGGCCGUUAGGCCGAA IAGGUUUU | 13788 |
| 1506 | AACCUCAC U GCCACUCU | 4449 | AGAGUGGC CUGAUGAGGCCGUUAGGCCGAA IUGAGGUG | 13789 |
| 1509 | CUCACUGC C ACUCUAAU | 4450 | AUUAGAGU CUGAUGAGGCCGUUAGGCCGAA ICAGUGAG | 13790 |
| 1510 | UCACUGCC A CUCUAAUU | 4451 | AAUUAGAG CUGAUGAGGCCGUUAGGCCGAA ICCAGUGA | 13791 |
| 1512 | ACUGCCAC U CUAAUUGU | 4452 | ACAAUUAG CUGAUGAGGCCGUUAGGCCGAA IUGGCAGU | 13792 |
| 1514 | UGCCACUC U AAUUGUCA | 4453 | UGACAAUU CUGAUGAGGCCGUUAGGCCGAA IAGUGUCA | 13793 |
| 1522 | UAAUUGUC A AUGUGAAA | 4454 | UUUCACAU CUGAUGAGGCCGUUAGGCCGAA IACAAUUA | 13794 |
| 1532 | UGUGAAAC C CCAGAUUU | 4455 | AAAUCUGG CUGAUGAGGCCGUUAGGCCGAA IUUUCACA | 13795 |
| 1533 | GUGAAACC C CAGAUUUA | 4456 | UAAAUCUG CUGAUGAGGCCGUUAGGCCGAA IGUUUCAC | 13796 |
| 1534 | UGAAACCC C AGAUUUAC | 4457 | GUAAAUCU CUGAUGAGGCCGUUAGGCCGAA IGGUUUCA | 13797 |
| 1535 | GAAACCCC A GAUUUACG | 4458 | CGUAAAUC CUGAUGAGGCCGUUAGGCCGAA IGGGUUUC | 13798 |
| 1551 | GAAAAGGC C GUGUCAUC | 4459 | GAUGACAC CUGAUGAGGCCGUUAGGCCGAA ICCUUUUC | 13799 |
| 1557 | GCCGUGUC A UCGUUUCC | 4460 | GGAAACGA CUGAUGAGGCCGUUAGGCCGAA IACACGGC | 13800 |
| 1565 | AUCGUUUC C AGACCCGG | 4461 | CCGGGUCU CUGAUGAGGCCGUUAGGCCGAA IAAACGAU | 13801 |
| 1566 | UCGUUUCC A GACCCGGC | 4462 | GCCGGGUC CUGAUGAGGCCGUUAGGCCGAA IGAAACGA | 13802 |
| 1570 | UUCCAGAC C CGCCUCUC | 4463 | GAGAGCCG CUGAUGAGGCCGUUAGGCCGAA IUCUGGAA | 13803 |
| 1571 | UCCAGACC C GGCUCUCU | 4464 | AGAGAGCC CUGAUGAGGCCGUUAGGCCGAA IGUCUGGA | 13804 |
| 1575 | GACCCGGC U CUCUACCC | 4465 | GGGUAGAG CUGAUGAGGCCGUUAGGCCGAA ICCGGGUC | 13805 |
| 1577 | CCCGGCUC U CUACCCAC | 4466 | GUGGGUAG CUGAUGAGGCCGUUAGGCCGAA IAGCCGGG | 13806 |
| 1579 | CGGCUCUC U ACCCACUG | 4467 | CAGUGGGU CUGAUGAGGCCGUUAGGCCGAA IAGAGCCG | 13807 |
| 1582 | CUCUCUAC C CACUGGGC | 4468 | GCCCAGUG CUGAUGAGGCCGUUAGGCCGAA IUAGAGAG | 13808 |
| 1583 | UCUCUACC C ACUGGGCA | 4469 | UGCCCAGU CUGAUGAGGCCGUUAGGCCGAA IGUAGAGA | 13809 |
| 1584 | CUCUACCC A CUGGGCAG | 4470 | CUGCCCAG CUGAUGAGGCCGUUAGGCCGAA IGGUAGAG | 13810 |
| 1586 | CUACCCAC U GGGCAGCA | 4471 | UGCUGCCC CUGAUGAGGCCGUUAGGCCGAA IUGGGUAG | 13811 |
| 1591 | CACUGGGC A GCAGACAA | 4472 | CUGUCUGC CUGAUGAGGCCGUUAGGCCGAA ICCCAGUG | 13812 |
| 1594 | UGGGCAGC A GACAAAUC | 4473 | GAUUUGUC CUGAUGAGGCCGUUAGGCCGAA ICUGCCCA | 13813 |
| 1598 | CAGCAGAC A AAUCCUGA | 4474 | UCAGGAUU CUGAUGAGGCCGUUAGGCCGAA IUCUGCUG | 13814 |
| 1603 | GACAAAUC C UGACUUGU | 4475 | ACAAGUCA CUGAUGAGGCCGUUAGGCCGAA IAUUUGUC | 13815 |
| 1604 | ACAAAUCC U GACUUGUA | 4476 | UACAAGUC CUGAUGAGGCCGUUAGGCCGAA IGAUUUGU | 13816 |
| 1608 | AUCCUGAC U UGUACCGC | 4477 | GCGGUACA CUGAUGAGGCCGUUAGGCCGAA IUCAGGAU | 13817 |
| 1614 | ACUUGUAC C GCAUAUGG | 4478 | CCAUAUGC CUGAUGAGGCCGUUAGGCCGAA IUACAAGU | 13818 |
| 1617 | UGUACCGC A UAUGGUAU | 4479 | AUACCAUA CUGAUGAGGCCGUUAGGCCGAA ICGGUACA | 13819 |
| 1627 | AUGGUAUC C CUCAACCU | 4480 | AGGUUGAG CUGAUGAGGCCGUUAGGCCGAA IAUACCAU | 13820 |
| 1628 | UGGUAUCC C UCAACCUA | 4481 | UAGGUUGA CUGAUGAGGCCGUUAGGCCGAA IGAUACCA | 13821 |
| 1629 | GGUAUCCC U CAACCUAC | 4482 | GUAGGUUG CUGAUGAGGCCGUUAGGCCGAA IGGAUACC | 13822 |
| 1631 | UAUCCCUC A ACCUACAA | 4483 | UUGUAGGU CUGAUGAGGCCGUUAGGCCGAA IAGGGAUA | 13823 |
| 1634 | CCCUCAAC C UACAAUCA | 4484 | UGAUUGUA CUGAUGAGGCCGUUAGGCCGAA IUUGAGGG | 13824 |
| 1635 | CCUCAACC U ACAAUCAA | 4485 | UUGAUUGU CUGAUGAGGCCGUUAGGCCGAA IGUUGAGG | 13825 |
| 1638 | CAACCUAC A AUCAAGUG | 4486 | CACUUGAU CUGAUGAGGCCGUUAGGCCGAA IUAGGUUG | 13826 |
| 1642 | CUACAAUC A AGUGGUUC | 4487 | GAACCACU CUGAUGAGGCCGUUAGGCCGAA IAUUGUAG | 13827 |
| 1651 | AGUGGUUC U GGCACCCC | 4488 | GGGGUGCC CUGAUGAGGCCGUUAGGCCGAA IAACCACU | 13828 |
| 1655 | GUUCUGGC A CCCCUGUA | 4489 | UACAGGGG CUGAUGAGGCCGUUAGGCCGAA ICCAGAAC | 13829 |
| 1657 | UCUGGCAC C CCUGUAAC | 4490 | GUUACAGG CUGAUGAGGCCGUUAGGCCGAA IUGCCAGA | 13830 |
| 1658 | CUGGCACC C CUGUAACC | 4491 | GGUUACAG CUGAUGAGGCCGUUAGGCCGAA IGUGCCAG | 13831 |
| 1659 | UGGCACCC C UGUAACCA | 4492 | UGGUUACA CUGAUGAGGCCGUUAGGCCGAA IGGUGCCA | 13832 |
| 1660 | GGCACCCC U GUAACCAU | 4493 | AUGGUUAC CUGAUGAGGCCGUUAGGCCGAA IGGGUGCC | 13833 |
| 1666 | CCUGUAAC C AUAAUCAU | 4494 | AUGAUUAU CUGAUGAGGCCGUUAGGCCGAA IUUACAGG | 13834 |
| 1667 | CUGUAACC A UAAUCAUU | 4495 | AAUGAUUA CUGAUGAGGCCGUUAGGCCGAA IGUUACAG | 13835 |
| 1673 | CCAUAAUC A UUCCGAAG | 4496 | CUUCGGAA CUGAUGAGGCCGUUAGGCCGAA IAUUAUGG | 13836 |
| 1677 | AAUCAUUC C GAAGCAAG | 4497 | CUUGCUUC CUGAUGAGGCCGUUAGGCCGAA IAAUGAUU | 13837 |
| 1683 | UCCGAAGC A AGGUGUGA | 4498 | UCACACCU CUGAUGAGGCCGUUAGGCCGAA ICUUCGGA | 13838 |
| 1693 | GGUGUGAC U UUUGUUCC | 4499 | GGAACAAA CUGAUGAGGCCGUUAGGCCGAA IUCACACC | 13839 |
| 1701 | UUUUGUUC C AAUAAUGA | 4500 | UCAUUAUU CUGAUGAGGCCGUUAGGCCGAA IAACAAAA | 13840 |
| 1702 | UUUGUUCC A AUAAUGAA | 4501 | UUCAUUAU CUGAUGAGGCCGUUAGGCCGAA IGAACAAA | 13841 |
| 1716 | GAAGAGUC C UUUAUCCU | 4502 | AGGAUAAA CUGAUGAGGCCGUUAGGCCGAA IACUCUUC | 13842 |
| 1717 | AAGAGUCC U UUAUCCUG | 4503 | CAGGAUAA CUGAUGAGGCCGUUAGGCCGAA IGACUCUU | 13843 |
| 1723 | CCUUUAUC C UGGAUGCU | 4504 | AGCAUCCA CUGAUGAGGCCGUUAGGCCGAA IAUAAAGG | 13844 |
| 1724 | CUUUAUCC U GGAUGCUG | 4505 | CAGCAUCC CUGAUGAGGCCGUUAGGCCGAA IGAUAAAG | 13845 |
| 1731 | CUGGAUGC U GACAGCAA | 4506 | UUGCUGUC CUGAUGAGGCCGUUAGGCCGAA ICAUCCAG | 13846 |
| 1735 | AUGCUGAC A GCAACAUG | 4507 | CAUGUUGC CUGAUGAGGCCGUUAGGCCGAA IUCAGCAU | 13847 |
| 1738 | CUGACAGC A ACAUGGGA | 4508 | UCCCAUGU CUGAUGAGGCCGUUAGGCCGAA ICUGUCAG | 13848 |
| 1741 | ACAGCAAC A UGGGAAAC | 4509 | GUUUCCCA CUGAUGAGGCCGUUAGGCCGAA IUUGCUGU | 13349 |
| 1750 | UGGGAAAC A GAAUUGAG | 4510 | CUCAAUUC CUGAUGAGGCCGUUAGGCCGAA IUUUCCCA | 13850 |
| 1762 | UUGAGAGC A UCACUCAG | 4511 | CUGAGUGA CUGAUGAGGCCGUUAGGCCGAA ICUCUCAA | 13851 |
| 1765 | AGAGCAUC A CUCAGCGC | 4512 | GCGCUGAG CUGAUGAGGCCGUUAGGCCGAA IAUGCUCU | 13852 |
| 1767 | ACCAUCAC U CAGCGCAU | 4513 | AUGCGCUG CUGAUGAGGCCGUUAGGCCGAA IUGAUGCU | 13853 |
| 1769 | CAUCACUC A GCGCAUGG | 4514 | CCAUGCGC CUGAUGAGGCCGUUAGGCCGAA IAGUGAUG | 13854 |
| 1774 | CUCAGCGC A UGGCAAUA | 4515 | UAUUGCCA CUGAUGAGGCCGUUAGGCCGAA ICGCUGAG | 13855 |
| 1779 | CGCAUGGC A AUAAUAGA | 4516 | UCUAUUAU CUGAUGAGGCCGUUAGGCCGAA ICCAUGCG | 13856 |
| 1806 | AAGAUGGC U AGCACCUU | 4517 | AAGGUGCU CUGAUGAGGCCGUUAGGCCGAA ICCAUCUU | 13857 |
| 1810 | UGGCUAGC A CCUUGGUU | 4518 | AACCAAGG CUGAUGAGGCCGUUAGGCCGAA ICUAGCCA | 13858 |
| 1812 | GCUAGCAC C UUGGUUGU | 4519 | ACAACCAA CUGAUGAGGCCGUUAGGCCGAA IUGCUAGC | 13859 |
| 1813 | CUAGCACC U UGGUUGUG | 4520 | CACAACCA CUGAUGAGGCCGUUAGGCCGAA IGUGCUAG | 13860 |
| 1824 | GUUGUGGC U GACUCUAG | 4521 | CUAGAGUC CUGAUGAGGCCGUUAGGCCGAA ICCACAAC | 13861 |
| 1828 | UGGCUGAC U CUAGAAUU | 4522 | AAUUCUAG CUGAUGAGGCCGUUAGGCCGAA IUCAGCCA | 13862 |
| 1830 | GCUGACUC U AGAAUUUC | 4523 | GAAAUUCU CUGAUGAGGCCGUUAGGCCGAA IAGUCAGC | 13863 |
| 1839 | AGAAUUUC U GGAAUCUA | 4524 | UAGAUUCC CUGAUGAGGCCGUUAGGCCGAA IAAAUUCU | 13864 |
| 1846 | CUGGAAUC U ACAUUUGC | 4525 | GCAAAUGU CUGAUGAGGCCGUUAGGCCGAA IAUUCCAG | 13865 |
| 1849 | GAAUCUAC A UUUGCAUA | 4526 | UAUGCAAA CUGAUGAGGCCGUUAGGCCGAA IUAGAUUC | 13866 |
| 1855 | ACAUUUGC A UAGCUUCC | 4527 | GGAAGCUA CUGAUGAGGCCGUUAGGCCGAA ICAAAUGU | 13867 |
| 1860 | UGCAUAGC U UCCAAUAA | 4528 | UUAUUGGA CUGAUGAGGCCGUUAGGCCGAA ICUAUGCA | 13868 |
| 1863 | AUAGCUUC C AAUAAAGU | 4529 | ACUUUAUU CUGAUGAGGCCGUUAGGCCGAA IAAGCUAU | 13869 |
| 1864 | UAGCUUCC A AUAAAGUU | 4530 | AACUUUAU CUGAUGAGGCCGUUAGGCCGAA IGAAGCUA | 13870 |
| 1878 | GUUGGGAC U GUGGGAAG | 4531 | CUUCCCAC CUGAUGAGGCCGUUAGGCCGAA IUCCCAAC | 13871 |
| 1891 | GAAGAAAC A UAAGCUUU | 4532 | AAAGCUUA CUGAUGAGGCCGUUAGGCCGAA IUUUCUUC | 13872 |
| 1897 | ACAUAAGC U UUUAUAUC | 4533 | GAUAUAAA CUGAUGAGGCCGUUAGGCCGAA ICUUAUGU | 13873 |
| 1906 | UUUAUAUC A CAGAUGUG | 4534 | CACAUCUG CUGAUGAGGCCGUUAGGCCGAA IAUAUAAA | 13874 |
| 1908 | UAUAUCAC A GAUGUGCC | 4535 | GGCACAUC CUGAUGAGGCCGUUAGGCCGAA IUGAUAUA | 13875 |
| 1916 | AGAUGUGC C AAAUGGGU | 4536 | ACCCAUUU CUGAUGAGGCCGUUAGGCCGAA ICACAUCU | 13876 |
| 1917 | GAUGUGCC A AAUGGGUU | 4537 | AACCCAUU CUGAUGAGGCCGUUAGGCCGAA IGCACAUC | 13877 |
| 1928 | UGGGUUUC A UGUUAACU | 4538 | AGUUAACA CUGAUGAGGCCGUUAGGCCGAA IAAACCCA | 13878 |
| 1936 | AUGUUAAC U UGGAAAAA | 4539 | UUUUUCCA CUGAUGAGGCCGUUAGGCCGAA IUUAACAU | 13879 |
| 1949 | AAAAAUGC C GACGGAAG | 4540 | CUUCCGUC CUGAUGAGGCCGUUAGGCCGAA ICAUUUUU | 13880 |
| 1966 | GAGAGGAC C UGAAACUG | 4541 | CAGUUUCA CUGAUGAGGCCGUUAGGCCGAA IUCCUCUC | 13881 |
| 1967 | AGAGGACC U GAAACUGU | 4542 | ACAGUUUC CUGAUGAGGCCGUUAGGCCGAA IGUCCUCU | 13882 |
| 1973 | CCUGAAAC U GUCUUGCA | 4543 | UGCAAGAC CUGAUGAGGCCGUUAGGCCGAA IUUUCAGG | 13883 |
| 1977 | AAACUGUC U UGCACAGU | 4544 | ACUGUGCA CUGAUGAGGCCGUUAGGCCGAA IACAGUUU | 13884 |
| 1981 | UGUCUUGC A CAGUUAAC | 4545 | GUUAACUG CUGAUGAGGCCGUUAGGCCGAA ICAAGACA | 13885 |
| 1983 | UCUUGCAC A GUUAACAA | 4546 | UUGUUAAC CUGAUGAGGCCGUUAGGCCGAA IUGCAAGA | 13886 |
| 1990 | CAGUUAAC A AGUUCUUA | 4547 | UAAGAACU CUGAUGAGGCCGUUAGGCCGAA IUUAACUG | 13887 |
| 1996 | ACAAGUUC U UAUACAGA | 4548 | UCUGUAUA CUGAUGAGGCCGUUAGGCCGAA IAACUUGU | 13888 |
| 2002 | UCUUAUAC A GAGACGUU | 4549 | AACGUCUC CUGAUGAGGCCGUUAGGCCGAA IUAUAAGA | 13889 |
| 2013 | GACGUUAC U UGGAUUUU | 4550 | AAAAUCCA CUGAUGAGGCCGUUAGGCCGAA IUAACGUC | 13890 |
| 2024 | GAUUUUAC U GCGGACAG | 4551 | CUGUCCGC CUGAUGAGGCCGUUAGGCCGAA IUAAAAUC | 13891 |
| 2031 | CUGCGGAC A GUUAAUAA | 4552 | UUAUUAAC CUGAUGAGGCCGUUAGGCCGAA IUCCGCAG | 13892 |
| 2041 | UUAAUAAC A GAACAAUG | 4553 | CAUUGUUC CUGAUGAGGCCGUUAGGCCGAA IUUAUUAA | 13893 |
| 2046 | AACAGAAC A AUGCACUA | 4554 | UAGUGCAU CUGAUGAGGCCGUUAGGCCGAA IUUCUGUU | 13894 |
| 2051 | AACAAUGC A CUACAGUA | 4555 | UACUGUAG CUGAUGAGGCCGUUAGGCCGAA ICAUUGUU | 13895 |
| 2053 | CAAUGCAC U ACAGUAUU | 4556 | AAUACUGU CUGAUGAGGCCGUUAGGCCGAA IUGCAUUG | 13896 |
| 2056 | UGCACUAC A GUAUUAGC | 4557 | GCUAAUAC CUGAUGAGGCCGUUAGGCCGAA IUAGUGCA | 13897 |
| 2065 | GUAUUAGC A AGCAAAAA | 4558 | UUUUUGCU CUGAUGAGGCCGUUAGGCCGAA ICUAAUAC | 13898 |
| 2069 | UAGCAAGC A AAAAAUGG | 4559 | CCAUUUUU CUGAUGAGGCCGUUAGGCCGAA ICUUGCUA | 13899 |
| 2079 | AAAAUGCC C AUCACUAA | 4560 | UUAGUGAU CUGAUGAGGCCGUUAGGCCGAA ICCAUUUU | 13900 |
| 2080 | AAAUGGCC A UCACUAAC | 4561 | CUHAGUGA CUGAUGAGGCCGUUAGGCCGAA IGCCAUUU | 13901 |
| 2083 | UGGCCAUC A CUAAGGAG | 4562 | CUCCUUAG CUGAUGAGGCCGUUAGGCCGAA IAUGGCCA | 13902 |
| 2085 | GCCAUCAC U AAGGAGCA | 4563 | UGCUCCUU CUGAUGAGGCCGUUAGGCCGAA IUGAUGGC | 13903 |
| 2093 | UAAGGAGC A CUCCAUCA | 4564 | UGAUGGAG CUGAUGAGGCCGUUAGGCCGAA ICUCCUUA | 13904 |
| 2095 | AGGAGCAC U CCAUCACU | 4565 | AGUGAUGG CUGAUGAGGCCGUUAGGCCGAA IUGCUCCU | 13905 |
| 2097 | GACCACUC C AUCACUCU | 4566 | AGAGUGAU CUGAUGAGGCCGUUAGGCCGAA IAGUGCUC | 13906 |
| 2098 | AGCACUCC A UCACUCUU | 4567 | AAGAGUGA CUGAUGAGGCCGUUAGGCCGAA IGAGUGCU | 13907 |
| 2101 | ACUCCAUC A CUCUUAAU | 4568 | AUUAAGAG CUGAUGAGGCCGUUAGGCCGAA IAUGGAGU | 13908 |
| 2103 | UCCAUCAC U CUUAAUCU | 4569 | AGAUUAAG CUGAUGAGGCCGUUAGGCCGAA IUGAUGGA | 13909 |
| 2105 | CAUCACUC U UAAUCUUA | 4570 | UAAGAUUA CUGAUGAGGCCGUUAGGCCGAA IAGUGAUG | 13910 |
| 2111 | UCUUAAUC U UACCAUCA | 4571 | UGAUGGUA CUGAUGAGGCCGUUAGGCCGAA IAUUAAGA | 13911 |
| 2115 | AAUCUUAC C AUCAUGAA | 4572 | UUCAUGAU CUGAUGAGGCCGUUAGGCCGAA IUAAGAUU | 13912 |
| 2116 | AUCUUACC A UCAUGAAU | 4573 | AUUCAUGA CUGAUGAGGCCGUUAGGCCGAA IGUAAGAU | 13913 |
| 2119 | GUACCAUC A UGAAUGUU | 4574 | AACAUUCA CUGAUGAGGCCGUUAGGCCGAA IAUGGUAA | 13914 |
| 2130 | AAUGUUUC C CUGCAAGA | 4575 | UCUUGCAG CUGAUGAGGCCGUUAGGCCGAA IAAACAUU | 13915 |
| 2131 | AUGUUUCC C UGCAAGAU | 4576 | AUCUUGCA CUGAUGAGGCCGUUAGGCCGAA IGAAACAU | 13916 |
| 2132 | UGUUUCCC U GCAAGAUU | 4577 | AAUCUUGC CUGAUGAGGCCGUUAGGCCGAA IGGAAACA | 13917 |
| 2135 | UUCCCUGC A AGAUUCAG | 4578 | CUGAAUCU CUGAUGAGGCCGUUAGGCCGAA ICAGGGAA | 13918 |
| 2142 | CAAGAUUC A GGCACCUA | 4579 | UAGGUGCC CUGAUGAGGCCGUUAGGCCGAA IAAUCUUG | 13919 |
| 2146 | AUUCAGGC A CCUAUGCC | 4580 | GGCAUAGG CUGAUGAGGCCGUUAGGCCGAA ICCUGAAU | 13920 |
| 2148 | UCAGGCAC C UAUGCCUG | 4581 | CAGGCAUA CUGAUGAGGCCGUUAGGCCGAA IUGCCUGA | 13921 |
| 2149 | CAGGCACC U AUGCCUGC | 4582 | GCAGGCAU CUGAUGAGGCCGUUAGGCCGAA IGUGCCUG | 13922 |
| 2154 | ACCUAUGC C UGCAGAGC | 4583 | GCUCUGCA CUGAUGAGGCCGUUAGGCCGAA ICAUAGGU | 13923 |
| 2155 | CCUAUGCC U GCAGAGCC | 4584 | GGCUCUGC CUGAUGAGGCCGUUAGGCCGAA IGCAUAGG | 13924 |
| 2158 | AUGCCUGC A GAGCCAGG | 4585 | CCUGGCUC CUGAUGAGGCCGUUAGGCCGAA ICAGGCAU | 13925 |
| 2163 | UGCAGAGC C AGGAAUGU | 4586 | ACAUUCCU CUGAUGAGGCCGUUAGGCCGAA ICUCUGCA | 13926 |
| 2164 | GCAGAGCC A GGAAUGUA | 4587 | UACAUUCC CUGAUGAGGCCGUUAGGCCGAA IGCUCUGC | 13927 |
| 2176 | AUGUAUAC A CAGGGGAA | 4588 | UUCCCCUG CUGAUGAGGCCGUUAGGCCGAA IUAUACAU | 13928 |
| 2178 | GUAUACAC A GGGGAAGA | 4589 | UCUUCCCC CUGAUGAGGCCGUUAGGCCGAA IUGUAUAC | 13929 |
| 2191 | AAGAAAUC C UCCAGAAG | 4590 | CUUCUGGA CUGAUGAGGCCGUUAGGCCGAA IAUUUCUU | 13930 |
| 2192 | AGAAAUCC U CCAGAAGA | 4591 | UCUUCUGG CUGAUGAGGCCGUUAGGCCGAA IGAUUUCU | 13931 |
| 2194 | AAAUCCUC C AGAAGAAA | 4592 | UUUCUUCU CUGAUGAGGCCGUUAGGCCGAA IAGGAUUU | 13932 |
| 2195 | AAUCCUCC A GAAGAAAG | 4593 | CUUUCUUC CUGAUGAGGCCGUUAGGCCGAA IGAGGAUU | 13933 |
| 2211 | GAAAUUAC A AUCAGAGA | 4594 | UCUCUGAU CUGAUGAGGCCGUUAGGCCGAA IUAAUUUC | 13934 |
| 2215 | UUACAAUC A GAGAUCAG | 4595 | CUGAUCUC CUGAUGAGGCCGUUAGGCCGAA IAUUGUAA | 13935 |
| 2222 | CAGAGAUC A GGAAGCAC | 4596 | GUGCUUCC CUGAUGAGGCCGUUAGGCCGAA IAUCUCUG | 13936 |
| 2229 | CAGGAAGC A CCAUACCU | 4597 | AGGUAUGG CUGAUGAGGCCGUUAGGCCGAA ICUUCCUG | 13937 |
| 2231 | GGAAGCAC C AUACCUCC | 4598 | CCAGGUAU CUGAUGAGGCCGUUAGGCCGAA IUGCUUCC | 13938 |
| 2232 | GAAGCACC A UACCUCCU | 4599 | AGGAGGUA CUGAUGAGGCCGUUAGGCCGAA IGUGCUUC | 13939 |
| 2236 | CACCAUAC C UCCUGCGA | 4600 | UCGCAGGA CUGAUGAGGCCGUUAGGCCGAA IUAUGGUG | 13940 |
| 2237 | ACCAUACC U CCUGCGAA | 4601 | UUCGCAGG CUGAUGAGGCCGUUAGGCCGAA IGUAUGGU | 13941 |
| 2239 | CAUACCUC C UGCGAAAC | 4602 | GUUUCGCA CUGAUGAGGCCGUUAGGCCGAA IAGGUAUG | 13942 |
| 2240 | AUACCUCC U GCGAAACC | 4603 | GGUUUCGC CUGAUGAGGCCGUUAGGCCGAA IGAGGUAU | 13943 |
| 2248 | UGCGAAAC C UCAGUGAU | 4604 | AUCACUGA CUGAUGAGGCCGUUAGGCCGAA IUUUCGCA | 13944 |
| 2249 | GCGAAACC U CAGUGAUC | 4605 | GAUCACUG CUGAUGAGGCCGUUAGGCCGAA IGUUUCGC | 13945 |
| 2251 | GAAACCUC A GUGAUCAC | 4606 | GUGAUCAC CUGAUGAGGCCGUUAGGCCGAA IAGGUUUC | 13946 |
| 2258 | CAGUGAUC A CACAGUGG | 4607 | CCACUGUG CUGAUGAGGCCGUUAGGCCGAA IAUCACUG | 13947 |
| 2260 | GUGAUCAC A CAGUGGCC | 4608 | GGCCACUG CUGAUGAGGCCGUUAGGCCGAA IUGAUCAC | 13948 |
| 2262 | GAUCACAC A GUGGCCAU | 4609 | AUGGCCAC CUGAUGAGGCCGUUAGGCCGAA IUGUGAUC | 13949 |
| 2268 | ACAGUGGC C AUCAGCAG | 4610 | CUGCUGAU CUGAUGAGGCCGUUAGGCCGAA ICCACUGU | 13950 |
| 2269 | CAGUGGCC A UCACCAGU | 4611 | ACUGCUGA CUGAUGAGGCCGUUAGGCCGAA IGCCACUG | 13951 |
| 2272 | UGGCCAUC A GCAGUUCC | 4612 | GGAACUGC CUGAUGAGGCCGUUAGGCCGAA IAUGGCCA | 13952 |
| 2275 | CCAUCAGC A GUUCCACC | 4613 | GGUCCAAC CUGAUGAGGCCGUUAGGCCGAA ICUGAUGG | 13953 |
| 2280 | AGCAGUUC C ACCACUUU | 4614 | AAAGUGGU CUGAUGAGGCCGUUAGGCCGAA IAACUCCU | 13954 |
| 2281 | GCAGUUCC A CCACUUUA | 4615 | UAAAGUCG CUGAUGAGGCCCUUACGCCGAA IGAACUGC | 13955 |
| 2283 | AGUUCCAC C ACUUUAGA | 4616 | UCUAAAGU CUGAUGAGGCCGUUAGGCCGAA IUGGAACU | 13956 |
| 2284 | GUUCCACC A CUUUAGAC | 4617 | GUCUAAAG CUGAUGAGGCCGUUAGGCCGAA IGUGGAAC | 13957 |
| 2286 | UCCACCAC U CUAGACUG | 4618 | CAGUCUAA CUGAUGAGGCCGUUAGGCCGAA IUGGUGGA | 13958 |
| 2293 | CUUUAGAC U GUCAUGCU | 4619 | AGCAUGAC CUGAUGAGGCCGUUAGGCCGAA IUCUAAAG | 13959 |
| 2297 | AGACUGUC A UGCUAAUG | 4620 | CAUUAGCA CUGAUGAGGCCGUUAGGCCGAA IACAGUCU | 13960 |
| 2301 | UGUCAUGC U AAUGGUGU | 4621 | ACACCAUU CUGAUGAGGCCGUUAGGCCGAA ICAUGACA | 13961 |
| 2311 | AUGGUGUC C CCGAGCCU | 4622 | AGGCUCGG CUGAUGAGGCCGUUAGGCCGAA IACACCAU | 13962 |
| 2312 | UGGUGUCC C CGAGCCUC | 4623 | GAGGCUCG CUGAUGAGGCCGUUAGGCCGAA IGACACCA | 13963 |
| 2313 | GGUGUCCC C GAUCCUCA | 4624 | UGAGGCUC CUGAUGAGGCCGUUAGGCCGAA IGGACACC | 13964 |
| 2318 | CCCCGAGC C UCAGAUCA | 4625 | UGAUCUGA CUGAUGAGGCCGUUAGGCCGAA ICUCGGGG | 13965 |
| 2319 | CCCGAGCC U CAGAUCAC | 4626 | GUGAUCUG CUGAUGAGGCCGUUAGGCCGAA IGCUCGGG | 13966 |
| 2321 | CGAGCCUC A GAUCACUU | 4627 | AAGUGAUC CUGAUGAGGCCGUUAGGCCGAA IAGGCUCG | 13967 |
| 2326 | CUCAGAUC A CUUGGUUU | 4628 | AAACCAAG CUGAUGAGGCCGUUAGGCCGAA IAUCUGAG | 13968 |
| 2328 | CAGAUCAC U UGGUUUAA | 4629 | UUAAACCA CUGAUGAGGCCGUUAGGCCGAA IUGAUCUG | 13969 |
| 2341 | UUAAAAAC A ACCACAAA | 4630 | UUUGUGGU CUGAUGAGGCCGUUAGGCCGAA IUUUUUAA | 13970 |
| 2344 | AAAACAAC C ACAAAAUA | 4631 | UAUUUUGU CUGAUGAGGCCGUUAGGCCGAA IUUGUUUU | 13971 |
| 2345 | AAACAACC A CAAAAUAC | 4632 | GUAUUUUG CUGAUGAGGCCGUUAGGCCGAA IUUGUUUU | 13972 |
| 2347 | ACAACCAC A AAAUACAA | 4633 | UUGUAUUU CUGAUGAGGCCGUUAGGCCGAA IUGGUUGU | 13973 |
| 2354 | CAAAAUAC A ACAAGAGC | 4634 | GCUCUUGU CUGAUGAGGCCGUUAGGCCGAA IUAUUUUG | 13974 |
| 2357 | AAUACAAC A AGAGCCUG | 4635 | CAGGCUCU CUGAUGAGGCCGUUAGGCCGAA IUUGUAUU | 13975 |
| 2363 | ACAAGAGC C UGGAAUUA | 4636 | UAAUUCCA CUGAUGAGGCCGUUAGGCCGAA ICUCUUGU | 13976 |
| 2364 | CAAGAGCC U GGAAUUAU | 4637 | AUAAUUCC CUGAUGAGGCCGUUAGGCCGAA IGCUCUUG | 13977 |
| 2381 | UUUAGGAC C AGGAAGCA | 4638 | UGCUUCCU CUGAUGAGGCCGUUAGGCCGAA IUCCUAAA | 13978 |
| 2382 | UUAGGACC A GGAAGCAG | 4639 | CUGCUUCC CUGAUGAGGCCGUUAGGCCGAA IGUCCUAA | 13979 |
| 2389 | CAGGAAGC A GCACGCUG | 4640 | CAGCGUGC CUGAUGAGGCCGUUAGGCCGAA ICUUCCUG | 13980 |
| 2392 | GAAGCAGC A CGCUGUUU | 4641 | AAACAGCG CUGAUGAGGCCGUUAGGCCGAA ICUGCUUC | 13981 |
| 2396 | CAGCACGC U GUUUAUUG | 4642 | CAAUAAAC CUGAUGAGGCCGUUAGGCCGAA ICCUCCUG | 13982 |
| 2413 | AAAGAGUC A CAGAAGAG | 4643 | CUCUUCUG CUGAUGAGGCCGUUAGGCCGAA IACUCUUU | 13983 |
| 2415 | AGAGUCAC A GAAGAGGA | 4644 | UCCUCUUC CUGAUGAGGCCGUUAGGCCGAA IUGACUCU | 13984 |
| 2434 | AAGGUGUC U AUCACUGC | 4645 | GCAGUGAU CUGAUGAGGCCGUUAGGCCGAA IACACCUU | 13985 |
| 2438 | UGUCUAUC A CUGCAAAG | 4646 | CUUUGCAG CUGAUGAGGCCGUUAGGCCGAA IAUAGACA | 13986 |
| 2440 | UCUAUCAC U GCAAAGCC | 4647 | GGCUUUGC CUGAUGAGGCCGUUAGGCCGAA IUGAUAGA | 13987 |
| 2443 | AUCACUGC A AAGCCACC | 4648 | GGUGGCUU CUGAUGAGGCCGUUAGGCCGAA ICAGUGAU | 13988 |
| 2448 | UGCAAAGC C ACCAACCA | 4649 | UGGUUGGU CUGAUGAGGCCGUUAGGCCGAA ICUUUGCA | 13989 |
| 2449 | GCAAAGCC A CCAACCAG | 4650 | CUGGUUGG CUGAUGAGGCCGUUAGGCCGAA IGCUUUGC | 13990 |
| 2451 | AAAGCCAC C AACCAGAA | 4651 | UUCUGGUU CUGAUGAGGCCGUUAGGCCGAA IUGGCUUU | 13991 |
| 2452 | AAGCCACC A ACCAGAAG | 4652 | CUUCUGGU CUGAUGAGGCCGUUAGGCCGAA IGUGGCUU | 13992 |
| 2455 | CCACCAAC C AGAAGGGC | 4653 | GCCCUUCU CUGAUGAGGCCGUUAGGCCGAA IUUGGUGG | 13993 |
| 2456 | CACCAACC A GAAGGGCU | 4654 | AGCCCUUC CUGAUGAGGCCGUUAGGCCGAA IGUUGGUG | 13994 |
| 2464 | AGAAGGGC U CUGUGGAA | 4655 | UUCCACAG CUGAUGAGGCCGUUAGGCCGAA ICCCUUCU | 13995 |
| 2466 | AAGGGCUC U GUGGAAAG | 4656 | CUUUCCAC CUGAUGAGGCCGUUAGGCCGAA IAGCCCUU | 13996 |
| 2478 | GAAAGUUC A GCAUACCU | 4657 | AGGUAUGC CUGAUGAGGCCGUUAGGCCGAA IAACUUUC | 13997 |
| 2481 | AGUUCAGC A UACCUCAC | 4658 | GUGAGGUA CUGAUGAGGCCGUUAGGCCGAA ICUGAACU | 13998 |
| 2485 | CAGCAUAC C UCACUGUU | 4659 | AACAGUGA CUGAUGAGGCCGUUAGGCCGAA IUAUGCUG | 13999 |
| 2486 | AGCAUACC U CACUGUUC | 4660 | GAACAGUG CUGAUGAGGCCGUUAGGCCGAA IGUAUGCU | 14000 |
| 2488 | CAUACCUC A CUGUUCAA | 4661 | UUGAACAG CUGAUGAGGCCGUUAGGCCGAA IAGGUAUG | 14001 |
| 2490 | UACCUCAC U GUUCAAGG | 4662 | CCUUGAAC CUGAUGAGGCCGUUAUGCCGAA IUGAGGUA | 14002 |
| 2495 | CACUGUUC A AGGAACCU | 4663 | AGGUUCCU CUGAUGAGGCCGUUAGGCCGAA IAACAGUG | 14003 |
| 2502 | CAAGGAAC C UCCGACAA | 4664 | UUGUCCGA CUGAUGAGGCCGUUAGGCCGAA IUUCCUUG | 14004 |
| 2503 | AAGGAACC U CCGACAAG | 4665 | CUUGUCCG CUGAUGAGGCCGUUAGGCCGAA IGUUCCUU | 14005 |
| 2509 | CCUCGGAC A AGUCUAAU | 4666 | AUUAGACU CUGAUGAGGCCGUUAGGCCGAA IUCCGAGG | 14006 |
| 2514 | CACAAGUC U AAUCUGGA | 4667 | UCCAGAUC CUGAUGAGGCCGUUAGGCCGAA IACUUGUC | 14007 |
| 2519 | GUCUAAUC U GGAGCUGA | 4668 | UCAGCUCC CUGAUGAGGCCGUUAGGCCGAA IAUUAGAC | 14008 |
| 2525 | UCUGGAGC U GAUCACUC | 4669 | GAGUGAUC CUGAUGAGGCCGUUAGGCCGAA ICUCCAGA | 14009 |
| 2530 | ACCUGAUC A CUCUAACA | 4670 | UGUUAGAG CUGAUGAGGCCGUUAGGCCGAA IAUCAGCU | 14010 |
| 2532 | CUGAUCAC U CUAACAUG | 4671 | CAUGUUAG CUGAUGAGGCCGUUAGGCCGAA IUGAUCAG | 14011 |
| 2534 | GAUCACUC U AACAUGCA | 4672 | UGCAUGUU CUGAUGAGGCCGUUAGGCCGAA IAGUGAUC | 14012 |
| 2538 | ACUCUAAC A UGCACCUG | 4673 | CAGGUGCA CUGAUGAGGCCGUUAGGCCGAA IUUAGAGU | 14013 |
| 2542 | UAACAUGC A CCUGUGUG | 4674 | CACACAGG CUGAUGAGGCCGUUAGGCCGAA ICAUGUUA | 14014 |
| 2544 | ACAUCCAC C UGUGUGGC | 4675 | GCCACACA CUGAUGAGGCCGUUAGGCCGAA IUGCAUGU | 14015 |
| 2545 | CAUGCACC U GUGUGGCU | 4676 | AGCCACAC CUGAUGAGGCCGUUAGGCCGAA IGUGCAUG | 14016 |
| 2553 | UGUGUGUC U GCGACUCU | 4677 | AGAGUCGC CUGAUGAGGCCGUUAGGCCGAA ICCACACA | 14017 |
| 2559 | GCUGCGAC U CUCUUCUG | 4678 | CAGAAGAG CUGAUGAGGCCGUUAGGCCGAA IUCGCAGC | 14018 |
| 2561 | UGCGACUC U CUUCUGGC | 4679 | GCCAGAAG CUGAUGAGGCCGUUAGGCCGAA IAGUCGCA | 14019 |
| 2563 | CGACUCUC U UCUGGCUC | 4680 | GAGCCAGA CUGAUGAGGCCGUUAGGCCGAA IAGAGUCG | 14020 |
| 2566 | CUCUCUUC U GGCUCCUA | 4681 | UAGGAGCC CUGAUGAGGCCGUUAGGCCGAA IAAGAGAG | 14021 |
| 2570 | CUUCUGGC U CCUAUUAA | 4682 | UUAAUAGG CUGAUGAGGCCGUUAGGCCGAA ICCAGAAG | 14022 |
| 2572 | UCUGGCUC C UAUUAACC | 4683 | GGUUAAUA CUGAUGAGGCCGUUAGGCCGAA IAGCCAGA | 14023 |
| 2573 | CUGGCUCC U AUUAACCC | 4684 | GGGUUAAU CUGAUGAGGCCGUUAGGCCGAA IGAGCCAG | 14024 |
| 2580 | CUAUUAAC C CUCCUUAU | 4685 | AUAAGGAG CUGAUGAGGCCGUUAGGCCGAA IUUAAUAG | 14025 |
| 2581 | UAUUAACC C UCCUUAUC | 4686 | GAUAAGGA CUGAUGAGGCCGUUAGGCCGAA IGUUAAUA | 14026 |
| 2582 | AUUAACCC U CCUUAUCC | 4687 | GGAUAAGG CUGAUGAGGCCGUUAGGCCGAA IGGUUAAU | 14027 |
| 2584 | UAACCCUC C UUAUCCGA | 4688 | UCGGAUAA CUGAUGAGGCCGUUAGGCCGAA IAGGGUUA | 14028 |
| 2585 | AACCCUCC U UAUCCGAA | 4689 | UUCGGAUA CUGAUGAGGCCGUUAGGCCGAA IGAGGGUU | 14029 |
| 2590 | UCCUUAUC C GAAAAAUG | 4690 | CAUUUUUC CUGAUGAGGCCGUUAGGCCGAA IAUAAGGA | 14030 |
| 2607 | AAAAGGUC U UCUUCUGA | 4691 | UCAGAAGA CUGAUGAGGCCGUUAGGCCGAA IACCUUUU | 14031 |
| 2610 | AGGUCUUC U UCUGAAAU | 4692 | AUUUCAGA CUGAUGAGGCCGUUAGGCCGAA IAAGACCU | 14032 |
| 2613 | UCUUCUUC U GAAAUAAA | 4693 | UUUAUUUC CUGAUGAGGCCGUUAGGCCGAA IAAGAAGA | 14033 |
| 2625 | AUAAAGAC U GACUACCU | 4694 | AGGUAGUC CUGAUGAGGCCGUUAGGCCGAA IUCUUUAU | 14034 |
| 2629 | AGACUGAC U ACCUAUCA | 4695 | UGAUAGGU CUGAUGAGGCCGUUAGGCCGAA IUCAGUCU | 14035 |
| 2632 | CUGACUAC C UAUCAAUU | 4696 | AAUUGAUA CUGAUGAGGCCGUUAGGCCGAA IUAGUCAG | 14036 |
| 2633 | UGACUACC U AUCAAUUA | 4697 | UAAUUGAU CUGAUGAGGCCGUUAGGCCGAA IGUAGUCA | 14037 |
| 2637 | UACCUAUC A AUUAUAAU | 4698 | AUUAUAAU CUGAUGAGGCCGUUAGGCCGAA IAUAGGUA | 14038 |
| 2650 | UAAUGGAC C CAGAUGAA | 4699 | UUCAUCUG CUGAUGAGGCCGUUAGGCCGAA IUCCAUUA | 14039 |
| 2651 | AAUGGACC C AGAUGAAG | 4700 | CUUCAUCU CUGAUGAGGCCGUUAGGCCGAA IGUCCAUU | 14040 |
| 2652 | AUGGACCC A GAUGAAGU | 4701 | ACUUCAUC CUGAUGAGGCCGUUAGGCCGAA IGGUCCAU | 14041 |
| 2663 | UGAAGUUC C UUUGGAUG | 4702 | CAUCCAAA CUGAUGAGGCCGUUAGGCCGAA IAACUUCA | 14042 |
| 2664 | GAAGUUCC U UUGGAUGA | 4703 | UCAUCCAA CUGAUGAGGCCGUUAGGCCGAA IGAACUUC | 14043 |
| 2675 | GGAUGAGC A GUGUGAGC | 4704 | GCUCACAC CUGAUGAGGCCGUUAGGCCGAA ICUCAUCC | 14044 |
| 2687 | UGAGCGGC U CCCUUAUG | 4705 | CAUAAGGG CUGAUGAGGCCGUUAGGCCGAA ICCGCUCA | 14045 |
| 2689 | AGCGGCUC C CUUAUGAU | 4706 | AUCAUAAG CUGAUGAGGCCGUUAGGCCGAA IAGCCGCU | 14046 |
| 2690 | GCGGCUCC C UUAUGAUG | 4707 | CAUCAUAA CUGAUGAGGCCGUUAGGCCGAA IGAGCCGC | 14047 |
| 2691 | CGGCUCCC U UAUGAUGC | 4708 | GCAUCAUA CUGAUGAGGCCGUUAGGCCGAA IGGAGCCG | 14048 |
| 2700 | UAUGAUGC C AGCAAGUG | 4709 | CACUUGCU CUGAUGAGGCCGUUAGGCCGAA ICAUCAUA | 14049 |
| 2701 | AUGAUGCC A GCAAGUGG | 4710 | CCACUUGC CUGAUGAGGCCGUUAGGCCGAA IGCAUCAU | 14050 |
| 2704 | AUGCCAGC A AGUGGGAG | 4711 | CUCCCACU CUGAUGAGGCCGUUAGGCCGAA ICUGGCAU | 14051 |
| 2718 | GAGUUUGC C CGGGAGAG | 4712 | CUCUCCCG CUGAUGAGGCCGUUAGGCCGAA ICAAACUC | 14052 |
| 2719 | AGUUUGCC C GGGAGAGA | 4713 | UCUCUCCC CUGAUGAGGCCGUUAGGCCGAA IGCAAACU | 14053 |
| 2729 | GGAGAGAC U UAAACUGC | 4714 | CCAGUUUA CUGAUGAGGCCGUUAGGCCGAA IUCUCUCC | 14054 |
| 2735 | ACUUAAAC U GGGCAAAU | 4715 | AUUUGCCC CUGAUGAGGCCGUUAGGCCGAA IUUUAAGU | 14055 |
| 2740 | AACUGGGC A AAUCACUU | 4716 | AAGUGAUU CUGAUGAGGCCGUUAGGCCGAA ICCCAGUU | 14056 |
| 2745 | GGCAAAUC A CUUGGAAG | 4717 | CUUCCAAG CUGAUGAGGCCGUUAGGCCGAA IAUUUGCC | 14057 |
| 2747 | CAAAUCAC U UGGAAGAG | 4718 | CUCUUCCA CUGAUGAGGCCGUUAGGCCGAA IUGAUUUG | 14058 |
| 2760 | AGAGGGGC U UUUGGAAA | 4719 | UUUCCAAA CUGAUGAGGCCGUUAGGCCGAA ICCCCUCU | 14059 |
| 2777 | AGUGGUUC A AUCAUCAG | 4720 | CUGAUGCU CUGAUGAGGCCGUUAGGCCGAA IAACCACU | 14060 |
| 2781 | GUUCAAGC A UCAGCAUU | 4721 | AAUGCUGA CUGAUGAGGCCGUUAGGCCGAA ICUUGAAC | 14061 |
| 2784 | CAAGCAUC A GCAUUUGG | 4722 | CCAAAUGC CUGAUGAGGCCGUUAGGCCGAA IAUGCUUG | 14062 |
| 2787 | GCAUCAGC A UUUGGCAU | 4723 | AUGCCAAA CUGAUGAGGCCGUUAGGCCGAA ICUGAUGC | 14063 |
| 2794 | CAUUUGGC A UUAAGAAA | 4724 | UUUCUUAA CUGAUGAGGCCGUUAGGCCGAA ICCAAAUG | 14064 |
| 2805 | AAGAAAUC A CCUACGUG | 4725 | CACGUAGG CUGAUGAGGCCGUUAGGCCGAA IAUUUCUU | 14065 |
| 2807 | GAAAUCAC C UACGUGCC | 4726 | GGCACGUA CUGAUGAGGCCGUUAGGCCGAA IUGAUUUC | 14066 |
| 2808 | AAAUCACC U ACGUGCCG | 4727 | CGGCACGU CUGAUGAGGCCGUUAGGCCGAA IGUGAUUU | 14067 |
| 2815 | CUACGUGC C GGACUGUG | 4728 | CACAGUCC CUGAUGAGGCCGUUAGGCCGAA ICACGUAG | 14068 |
| 2820 | UGCCGGAC U GUGGCUGU | 4729 | ACAGCCAC CUGAUGAGGCCGUUAGGCCGAA IUCCGGCA | 14069 |
| 2826 | ACUGUGGC U GUGAAAAU | 4730 | AUUUUCAC CUGAUGAGGCCGUUAGGCCGAA ICCACAGU | 14070 |
| 2837 | GAAAAUGC U GAAAGAGG | 4731 | CGUCUUUC CUGAUGAGGCCGUUAGGCCGAA ICAUUUUC | 14071 |
| 2850 | GAGGGGGC C ACGGCCAG | 4732 | CUGGCCGU CUGAUGAGGCCGUUAGGCCGAA ICCCCCUC | 14072 |
| 2851 | AGGGGGCC A CGGCCAGC | 4733 | GCUGGCCG CUGAUGAGGCCGUUAGGCCGAA IGCCCCCU | 14073 |
| 2856 | GCCACGGC C AGCGAGUA | 4734 | UACUCGCU CUGAUGAGGCCGUUAGGCCGAA ICCGUGGC | 14074 |
| 2857 | CCACGGCC A GCGAGUAC | 4735 | GUACUCGC CUGAUGAGGCCGUUAGGCCGAA IGCCGUGG | 14075 |
| 2866 | GCGAGUAC A AAGCUCUG | 4736 | CAGAGCUU CUGAUGAGGCCGUUAGGCCGAA IUACUCGC | 14076 |
| 2871 | UACAAAGC U CUGAUGAC | 4737 | GUCAUCAG CUGAUGAGGCCGUUAGGCCGAA ICUUUGUA | 14077 |
| 2873 | CAAAGCUC U GAUGACUG | 4738 | CAGUCAUC CUGAUGAGGCCGUUAGGCCGAA IAGCUUUG | 14078 |
| 2880 | CUGAUGAC U GAGCUAAA | 4739 | UUUAGCUC CUGAUGAGGCCGUUAGGCCGAA IUCAUCAG | 14079 |
| 2885 | GACUGAGC U AAAAAUCU | 4740 | AGAUUUUU CUGAUGAGGCCGUUAGGCCGAA ICUCAGUC | 14080 |
| 2893 | UAAAAAUC U UGACCCAC | 4741 | GUGGGUCA CUGAUGAGGCCGUUAGGCCGAA IAUUUUUA | 14081 |
| 2898 | AUCUUGAC C CACAUUGG | 4742 | CCAAUGUG CUGAUGAGGCCGUUAGGCCGAA IUCAAGAU | 14082 |
| 2899 | UCUUGACC C ACAUUGGC | 4743 | GCCAAUGU CUGAUGAGGCCGUUAGGCCGAA IGUCAAGA | 14083 |
| 2900 | CUUGACCC A CAUUGGCC | 4744 | GGCCAAUG CUGAUGAGGCCGUUAGGCCGAA IGGUCAAG | 14084 |
| 2902 | UGACCCAC A UUGGCCAC | 4745 | GUGGCCAA CUGAUGAGGCCGUUAGGCCGAA IUGGGUCA | 14085 |
| 2908 | ACAUUGGC C ACCAUCUG | 4746 | CAGAUGGU CUGAUGAGGCCGUUAGGCCGAA ICCAAUGU | 14086 |
| 2909 | CAUUGGCC A CCAUCUGA | 4747 | UCAGAUGG CUGAUGAGGCCGUUAGGCCGAA IGCCAAUG | 14087 |
| 2911 | UUGGCCAC C AUCUGAAC | 4748 | GUUCAGAU CUGAUGAGGCCGUUAGGCCGAA IUGGCCAA | 14088 |
| 2912 | UGGCCACC A UCUGAACG | 4749 | CGUUCAGA CUGAUGAGGCCGUUAGGCCGAA IGUGGCCA | 14089 |
| 2915 | CCACCAUC U GAACGUGG | 4750 | CCACGUUC CUGAUGAGGCCGUUAGGCCGAA IAUGGUGG | 14090 |
| 2929 | UGGUUAAC C UGCUGGGA | 4751 | UCCCAGCA CUGAUGAGGCCGUUAGGCCGAA IUUAACCA | 14091 |
| 2930 | GGUUAACC U GCUGGGAG | 4752 | CUCCCAGC CUGAUGAGGCCGUUAGGCCGAA IGUUAACC | 14092 |
| 2933 | UAACCUGC U GGGAGCCU | 4753 | AGGCUCCC CUGAUGAGGCCGUUAGGCCGAA ICAGGUUA | 14093 |
| 2940 | CUGGGAGC C UGCACCAA | 4754 | UUGGUGCA CUGAUGAGGCCGUUAGGCCGAA ICUCCCAG | 14094 |
| 2941 | UGGGAGCC U GCACCAAG | 4755 | CUUGGUGC CUGAUGAGGCCGUUAGGCCGAA IGCUCCCA | 14095 |
| 2944 | GAGCCUGC A CCAAGCAU | 4756 | UUGCUUGG CUGAUGAGGCCGUUAGGCCGAA ICAGGCUC | 14096 |
| 2946 | GCCUGCAC C AAGCAAGG | 4757 | CCUUGCUU CUGAUGAGGCCGUUAGGCCGAA IUGCAGGC | 14097 |
| 2947 | CCUGCACC A AGCAAGGA | 4758 | UCCUUGCU CUGAUGAGGCCGUUAGGCCGAA IGUGCAGG | 14098 |
| 2951 | CACCAAGC A AGGAGGGC | 4759 | GCCCUCCU CUGAUGAGGCCGUUAGGCCGAA ICUUGGUG | 14099 |
| 2960 | AGGAGGGC C UCUGAUGG | 4760 | CCAUCAGA CUGAUGAGGCCGUUAGGCCGAA ICCCUCCU | 14100 |
| 2961 | GGAGGGCC U CUGAUGGU | 4761 | ACCAUCAG CUGAUGAGGCCGUUAGGCCGAA IGCCCUCC | 14101 |
| 2963 | AGGGCCUC U GAUGGUGA | 4762 | UCACCAUC CUGAUGAGGCCGUUAGGCCGAA IAGGCCCU | 14102 |
| 2983 | UUGAAUAC U GCAAAUAU | 4763 | AUAUUUGC CUGAUGAGGCCGUUAGGCCGAA IUAUUCAA | 14103 |
| 2986 | AAUACUGC A AAUAUGGA | 4764 | UCCAUAUU CUGAUGAGGCCGUUAGGCCGAA ICAGUAUU | 14104 |
| 2999 | UGGAAAUC U CUCCAACU | 4765 | AGUUGGAG CUGAUGAGGCCGUUAGGCCGAA IAUUUCCA | 14105 |
| 3001 | GAAAUCUC U CCAACUAC | 4766 | GUAGUUGG CUGAUGAGGCCGUUAGGCCGAA IAGAUUUC | 14106 |
| 3003 | AAUCUCUC C AACUACCU | 4767 | AGGUAGUU CUGAUGAGGCCGUUAGGCCGAA IAGAGAUU | 14107 |
| 3004 | AUCUCUCC A ACUACCUC | 4768 | GAGGUAGU CUUAUGAGGCCGUUAGGCCGAA IGAGAGAU | 14108 |
| 3007 | UCUCCAAC U ACCUCAAG | 4769 | CUUGAGGU CUGAUGAGGCCGUUAGGCCGAA IUUGGAGA | 14109 |
| 3010 | CCAACUAC C UCAAGAGC | 4770 | GCUCUUGA CUGAUGAGGCCGUUAGGCCGAA IUAGUUGG | 14110 |
| 3011 | CAACUACC U CAAGAGCA | 4771 | UGCUCUUG CUGAUGAGGCCGUUAGGCCGAA IGUAGUUG | 14111 |
| 3013 | ACUACCUC A AGAGCAAA | 4772 | UUUGCUCU CUGAUGAGGCCGUUAGGCCGAA IAGGUAGU | 14112 |
| 3019 | UCAAGAGC A AACGUGAC | 4773 | GUCACGUU CUGAUGAGGCCGUUAGGCCGAA ICUCUUGA | 14113 |
| 3028 | AACGUGAC U UAUUUUUU | 4774 | AAAAAAUA CUGAUGAGGCCGUUAGGCCGAA IUCACGUU | 14114 |
| 3038 | AUUUUUUC U CAACAAGG | 4775 | CCUUGUUG CUGAUGAGGCCGUUAGGCCGAA IAAAAAAU | 14115 |
| 3040 | UUUUUCUC A ACAAGGAU | 4776 | AUCCUUGU CUGAUGAGGCCGUUAGGCCGAA IAGAAAAA | 14116 |
| 3043 | UUCUCAAC A AGGAUGCA | 4777 | UGCAUCCU CUGAUGAGGCCGUUAGGCCGAA IUUGAGAA | 14117 |
| 3051 | AAGGAUGC A GCACUACA | 4778 | UGUAGUGC CUGAUGAGGCCGUUAGGCCGAA ICAUCCUU | 14118 |
| 3054 | GAUGCAGC A CUACACAU | 4779 | AUGUGUAG CUGAUGAGGCCGUUAGGCCGAA ICUGCAUC | 14119 |
| 3056 | UGCAGCAC U ACACAUGG | 4780 | CCAUGUGU CUGAUGAGGCCGUUAGGCCGAA IUGCUGCA | 14120 |
| 3059 | AGCACUAC A CAUGGAGC | 4781 | GCUCCAUG CUGAUGAGGCCGUUAGGCCGAA IUAGUGCU | 14121 |
| 3061 | CACUACAC A UGGAGCCU | 4782 | AGGCUCCA CUGAUGAGGCCGUUAGGCCGAA IUGUAGUG | 14122 |
| 3068 | CAUGGAGC C UAAGAAAG | 4783 | CUUUCUUA CUGAUGAGGCCGUUAGGCCGAA ICUCCAUG | 14123 |
| 3069 | AUGGAGCC U AAGAAAGA | 4784 | UCUUUCUU CUGAUGAGGCCGUUAGGCCGAA IGCUCCAU | 14124 |
| 3089 | AAUGGAGC C AGGCCUGG | 4785 | CCAGGCCU CUGAUGAGGCCGUUAGGCCGAA ICUCCAUU | 14125 |
| 3090 | AUGGAGCC A GGCCUGGA | 4786 | UCCAGGCC CUGAUGAGGCCGUUAGGCCGAA IGCUCCAU | 14126 |
| 3094 | AGCCAGGC C UGGAACAA | 4787 | UUGUUCCA CUGAUGAGGCCGUUAGGCCGAA ICCUGGCU | 14127 |
| 3095 | GCCAGGCC U GGAACAAG | 4788 | CUUGUUCC CUGAUGAGGCCGUUAGGCCGAA IGCCUGGC | 14128 |
| 3101 | CCUGGAAC A AGGCAAGA | 4789 | UCUUGCCU CUGAUGAGGCCGUUAGGCCGAA IUUCCAGG | 14129 |
| 3106 | AACAAGGC A AGAAACCA | 4790 | UGGUUUCU CUGAUGAGGCCGUUAGGCCGAA ICCUUGUU | 14130 |
| 3113 | CAAGAAAC C AAGACUAG | 4791 | CUAGUCUU CUGAUGAGGCCGUUAGGCCGAA IUUUCUUG | 14131 |
| 3114 | AAGAAACC A AGACUAGA | 4792 | UCUAGUCU CUGAUGAGGCCGUUAGGCCGAA IGUUUCUU | 14132 |
| 3119 | ACCAAGAC U AGAUAGCG | 4793 | CGCUAUCU CUGAUGAGGCCGUUAGGCCGAA IUCUUGGU | 14133 |
| 3130 | AUAGCGUC A CCAGCAGC | 4794 | GCUGCUGG CUGAUGAGGCCGUUAGGCCGAA IACGCUAU | 14134 |
| 3132 | AGCUUCAC C AGCAGCGA | 4795 | UCGCUGCU CUGAUGAGGCCGUUAUGCCGAA IUGACGCU | 14135 |
| 3133 | GCGUCACC A GCAGCGAA | 4796 | UUCGCUGC CUGAUGAGGCCGUUAGGCCGAA IGUGACGC | 14136 |
| 3136 | UCACCAGC A GCGAAAGC | 4797 | GCUUUCGC CUGAUGAGGCCGUUAGGCCGAA ICUGGUGA | 14137 |
| 3145 | GCGAAAGC U UUGCGAGC | 4798 | GCUCGCAA CUGAUGAGGCCGUUAGGCCGAA ICUUUCGC | 14138 |
| 3154 | UUGCGAGC U CCGGCUUU | 4799 | AAAGCCGG CUGAUGAGGCCGUUAGGCCGAA ICUCGCAA | 14139 |
| 3156 | GCGAGCUC C GGCUUUCA | 4800 | UGAAAGCC CUGAUGAGGCCGUUAGGCCGAA IAGCUCGC | 14140 |
| 3160 | GCUCCGGC U UUCAGGAA | 4801 | UUCCUGAA CUGAUGAGGCCGUUAGGCCGAA ICCGGAGC | 14141 |
| 3164 | CGGCUUUC A GGAAGAUA | 4802 | UAUCUUCC CUGAUGAGGCCGUUAGGCCGAA IAAAGCCG | 14142 |
| 3179 | UAAAAGUC U GAGUGAUG | 4803 | CAUCACUC CUGAUGAGGCCGUUAGGCCGAA IACUUUUA | 14143 |
| 3207 | GAGGADUC U GACGGUUU | 4804 | AAACCGUC CUGAUGAGGCCGUUAGGCCGAA IAAUCCUC | 14144 |
| 3217 | ACGGUUUC U ACAAGGAG | 4805 | CUCCUUGU CUGAUGAGGCCGUUAGGCCGAA IAAACCGU | 14145 |
| 3220 | GUUUCUAC A AGGAGCCC | 4806 | GGGCUCCU CUGAUGAGGCCGUUAGGCCGAA IUAGAAAC | 14146 |
| 3227 | CAAGGAGC C CAUCACUA | 4807 | UAGUGAUG CUGAUGAGGCCGUUAGGCCGAA ICUCCUUG | 14147 |
| 3228 | AAGGAGCC C AUCACUAU | 4808 | AUAGUGAU CUGAUGAGGCCGUUAGGCCGAA IGCUCCUU | 14148 |
| 3229 | AGGAGCCC A UCACUAUG | 4809 | CAUAGUGA CUGAUGAGGCCGUUAGGCCGAA IGGCUCCU | 14149 |
| 3232 | AGCCCAUC A CUAUGGAA | 4810 | UUCCAUAG CUGAUGAGGCCGUUAGGCCGAA IAUGGGCU | 14150 |
| 3234 | CCCAUCAC U AUGGAAGA | 4811 | UCUUCCAU CUGAUGAGGCCGUUAGGCCGAA IUGAUGGG | 14151 |
| 3245 | GGAAGAUC U GAUUUCUU | 4812 | AAGAAAUC CUGAUGAGGCCGUUAGGCCGAA IAUCUUCC | 14152 |
| 3252 | CUGAUUUC U UACAGUUU | 4813 | AAACUGUA CUGAUGAGGCCGUUAGGCCGAA IAAAUCAG | 14153 |
| 3256 | UUUCUUAC A GUUUUCAA | 4814 | UUGAAAAC CUGAUGAGGCCGUUAGGCCGAA IUAAGAAA | 14154 |
| 3263 | CACUUUUC A AGUGGCCA | 4815 | UGGCCACU CUGAUGAGGCCGUUAGGCCGAA IAAAACUG | 14155 |
| 3270 | CAAGUGGC C AGACGCAU | 4816 | AUGCCUCU CUGAUGAGGCCGUUAGGCCGAA ICCACUUC | 14156 |
| 3271 | AAGUGGCC A GAGGCAUG | 4817 | CAUGCCUC CUGAUGAGGCCGUUAGGCCGAA IGCCACUU | 14157 |
| 3277 | CCAGAGGC A UGGAGUUC | 4818 | GAACUCCA CUCAUGAGGCCCUUAGGCCGAA ICCUCUGG | 14158 |
| 3286 | UGGAGUUC C UGUCUUCC | 4819 | GGAAGACA CUGAUGAGGCCGUUAGGCCGAA IAACUCCA | 14159 |
| 3287 | GGAGUUCC U GUCUUCCA | 4820 | UGGAAGAC CUGAUGAGGCCGUUAGGCCGAA IGAACUCC | 14160 |
| 3291 | CUCCUGUC U UCCAGAAA | 4821 | UUUCUGGA CUGAUGAGGCCGUUAGGCCGAA IACAGGAA | 14161 |
| 3294 | CUGUCUUC C AGAAAGUG | 4822 | CACUUUCU CUGAUGAGGCCGUUAGGCCGAA IAAGACAG | 14162 |
| 3295 | UGUCUUCC A GAAAGUGC | 4823 | GCACUUUC CUGAUGAGGCCGUUAGGCCGAA IGAAGACA | 14163 |
| 3304 | GAAAGUGC A UUCAUCGG | 4824 | CCGAUGAA CUGAUGAGGCCGUUAGGCCGAA ICACUUUC | 14164 |
| 3308 | GUGCAUUC A UCGGGACC | 4825 | GGUCCCGA CUGAUGAGGCCGUUAGGCCGAA IAAUGCAC | 14165 |
| 3316 | AUCGGGAC C UGGCAGCG | 4826 | CGCUGCCA CUGAUGAGGCCGUUAGGCCGAA IUCCCGAU | 14166 |
| 3317 | UCGGGACC U GGCAGCGA | 4827 | UCGCUGCC CUGAUGAGGCCGUUAGGCCGAA IGUCCCGA | 14167 |
| 3321 | GACCUGGC A GCGAGAAU | 4828 | UUUCUCGC CUGAUGAGGCCGUUAGGCCGAA ICCAGGUC | 14168 |
| 3331 | CGAGAAAC A UUCUUUUA | 4829 | UAAAAGAA CUGAUGAGGCCGUUAGGCCGAA IUUUCUCG | 14169 |
| 3335 | AAACAUCU U UUCAUCUG | 4830 | CAGAUAAA CUGAUGAGGCCGUUAGGCCGAA IAAUGUUU | 14170 |
| 3342 | CUUUUAUC U GAGAACAA | 4831 | UUGUUCUC CUGAUGAGGCCGUUAGGCCGAA IAUAAAAG | 14171 |
| 3349 | CUGAGAAC A ACCUGGUG | 4832 | CACCACGU CUGAUGAGGCCGUUAGGCCGAA IDUCUCAG | 14172 |
| 3376 | AUUUUGGC C UUGCCCGG | 4833 | CCGGGCAA CUGAUGAGGCCGUUAGGCCGAA ICCAAAAU | 14173 |
| 3377 | UUUUGGCC U UGCCCGGG | 4834 | CCCGGGCA CUGAUGAGGCCGUUAGGCCGAA IGCCAAAA | 14174 |
| 3381 | GGCCUUGC C CGGGAUAU | 4835 | AUAUCCCG CUGAUGAGGCCGUUAGGCCGAA ICAAGGCC | 14175 |
| 3382 | GCCUUGCC C GGGAUAUU | 4836 | AAUAUCCC CUGAUGAGGCCGUUAGGCCGAA IGCAAGGC | 14176 |
| 3400 | AUAAGAAC C CCGAUUAU | 4837 | AUAAUCGG CUGAUGAGGCCGUUAGGCCGAA IUUCUUAU | 14177 |
| 3401 | UAAGAACC C CGAUUAUG | 4838 | CAUAAUCG CUGAUGAGGCCGUUAGGCCGAA IGUUCUUA | 14178 |
| 3402 | AAGAACCC C GAUUAUGU | 4839 | ACAUAAUC CUGAUGAGGCCGUUAGGCCGAA IGGUUCUU | 14179 |
| 3426 | GGAGAUAC U CGACUUCC | 4840 | GGAAGUCG CUGAUGAGGCCGUUAGGCCGAA IUAUCUCC | 14180 |
| 3431 | UACUCGAC U UCCUCUGA | 4841 | UCAGAGGA CUGAUGAGGCCGUUAGGCCGAA IUCGAGUA | 14181 |
| 3434 | UCGACUUC C UCUGAAAU | 4842 | AUUUCAGA CUGAUGAGGCCGUUAGGCCGAA IAAGUCGA | 14182 |
| 3435 | CGACUUCC U CUGAAAUG | 4843 | CAUWUCAG CUGAUGAGGCCGUUAGGCCGAA IGAAGUCG | 14183 |
| 3437 | ACUUCCUC U GAAAUGGA | 4844 | UCCAUUUC CUGAUGAGGCCGUUAGGCCGAA IAGGAAGU | 14184 |
| 3450 | UGGAUGGC U CCCGAAUC | 4845 | GAUUCGGG CUGAUGAGGCCGUUAGGCCGAA ICCAUCCA | 14185 |
| 3452 | GAUGGCUC C CGAAUCUA | 4846 | UAGAUUCG CUGAUGAGGCCGUUAGGCCGAA IAGCCAUC | 14186 |
| 3453 | AUGGCUCC C GAAUCUAU | 4847 | AUAGAUUC CUGAUGAGGCCGUUAGGCCGAA IGAGCCAU | 14187 |
| 3459 | CCCGAAUC U AUCUUUGA | 4848 | UCAAAGAU CUGAUGAGGCCGUUAGGCCGAA IAUUCGGG | 14188 |
| 3463 | AAUCUAUC U UUGACAAA | 4849 | UUUGUCAA CUGAUGAGGCCGUUAGGCCGAA IAUAGAUU | 14189 |
| 3469 | UCUUUGAC A AAAUCUAC | 4850 | GUAGAUUU CUGAUGAGGCCGUUAGGCCGAA IUCAAAGA | 14190 |
| 3475 | ACAAAAUC U ACAGCACC | 4851 | GGUGCUGU CUGAUGAGGCCGUUAGGCCGAA IAUUUUGU | 14191 |
| 3478 | AAAUCUAC A GCACCAAG | 4852 | CUUGGUGC CUGAUGAGGCCGUUAGGCCGAA IUAGAUUU | 14192 |
| 3481 | UCUACAGC A CCAAGAGC | 4853 | GCUCUUGG CUGAUGAGGCCGUUAGGCCGAA ICUGUAGA | 14193 |
| 3483 | UACAGCAC C AAGAGCGA | 4854 | UCUCUCUU CUGAUGAGGCCGUUAGGCCGAA IUGCUGUA | 14194 |
| 3484 | ACAGCACC A AGAGCGAC | 4855 | GUCGCUCU CUGAUGAGGCCGUUAGGCCGAA IGUGCUGU | 14195 |
| 3501 | GUGUGGUC U UACGGAGU | 4856 | ACUCCGUA CUGAUGAGGCCGUUAGGCCGAA IACCACAC | 14196 |
| 3515 | AGUAUUGC U GUGGGAAA | 4857 | UUUCCCAC CUGAUGAGGCCGUUAGGCCGAA ICAAUACU | 14197 |
| 3526 | GGGAAAUC U UCUCCUUA | 4858 | UAAGGAGA CUGAUGAGGCCGUUAGGCCGAA IAUUUCCC | 14198 |
| 3529 | AAAUCUUC U CCUUAGGU | 4859 | ACCUAAGG CUGAUGAGGCCGUUAGGCCGAA IAAGAUUU | 14199 |
| 3531 | AUCUUCUC C UUAGGUGG | 4860 | CCACCUAA CUGAUGAGGCCGUUAGGCCGAA IAGAAGAU | 14200 |
| 3532 | UCUUCUCC U UAGGUGGG | 4861 | CCCACCUA CUGAUGAGGCCGUUAGGCCGAA IGAGAAGA | 14201 |
| 3543 | GGUGGGUC U CCAUACCC | 4862 | GGGUAUGG CUGAUGAGGCCGUUAGGCCGAA IACCCACC | 14202 |
| 3545 | UGGGUCUC C AUACCCAG | 4863 | CUGGGUAU CUGAUGAGGCCGUUAGGCCGAA IAGACCCA | 14203 |
| 3546 | GGGUCUCC A UACCCAGG | 4864 | CCUGGGUA CUGAUGAGGCCGUUAGGCCGAA IGAGACCC | 14204 |
| 3550 | CUCCAUAC C CAGGAGUA | 4865 | UACUCCUG CUGAUGAGGCCGUUAGGCCGAA IUAUGGAG | 14205 |
| 3551 | UCCAUACC C AGGAGUAC | 4866 | GUACUCCU CUGAUGAGGCCGUUAGGCCGAA IGUAUGGA | 14206 |
| 3552 | CCAUACCC A GUAGUACA | 4867 | UGUACUCC CUGAUGAGGCCGUUAGGCCGAA IGGUAUGG | 14207 |
| 3560 | AGGAGUAC A AAUGGAUG | 4868 | CAUCCAUU CUGAUGAGGCCGUUAGGCCGAA IUACUCCU | 14208 |
| 3574 | AUGAGGAC U UUUGCAGU | 4869 | ACUGCAAA CUGAUGAGGCCGUUAGGCCGAA IUCCUCAU | 14209 |
| 3580 | ACUUUUGC A GUCGCCUG | 4870 | CAGGCGAC CUGAUGAGGCCGUUAGGCCGAA ICAAAAGU | 14210 |
| 3586 | UCAGUCUC C UGAGGGAA | 4871 | UUCCCUCA CUGAUGAGGCCGUUAGGCCGAA ICGACUGC | 14211 |
| 3587 | CAGUCGCC U GAGGGAAG | 4872 | CUUCCCUC CUGAUGAGGCCGUUAGGCCGAA IGCGACUG | 14212 |
| 3598 | GGGAAGGC A UGAGGAUG | 4873 | CAUCCUCA CUGAUGAGGCCGUUAGGCCGAA ICCUUCCC | 14213 |
| 3612 | AUGAGAGC U CCUGAGUA | 4874 | UACUCAGG CUGAUGAGGCCGUUAGGCCGAA ICUCUCAU | 14214 |
| 3614 | GAGAGCUC C UGAGUACU | 4875 | AGUACUCA CUGAUGAGGCCGUUAGGCCGAA IAUCUCUC | 14215 |
| 3615 | AGAGCUCC U GAGUACUC | 4876 | GAGUACUC CUGAUGAGGCCGUUAGGCCGAA IGAGCUCU | 14216 |
| 3622 | CUGAGUAC U CUACUCCU | 4877 | AGGAGUAG CUGAUGAGGCCGUUAGGCCGAA IUACUCAG | 14217 |
| 3624 | GAGUACUC U ACUCCUGA | 4878 | UCAGGAGU CUGAUGAGGCCGUUAGGCCGAA IAGUACUC | 14218 |
| 3627 | UACUCUAC U CCUGAAAU | 4879 | AUUUCAGG CUGAUGAGGCCGUUAGGCCGAA IUAGAGUA | 14219 |
| 3629 | CUCUACUC C UGAAAUCU | 4880 | AGAUUUCA CUGAUGAGGCCGUUAGGCCGAA IAGUAGAG | 14220 |
| 3630 | UCUACUCC U GAAAUCUA | 4881 | UAGAUUUC CUGAUGAGGCCGUUAGGCCGAA IGAGUAGA | 14221 |
| 3637 | CUGAAAUC U AUCAGAUC | 4882 | GAUCUGAU CUGAUGAGGCCGUUAGGCCGAA IAUUUCAG | 14222 |
| 3641 | AAUCUAUC A GAUCAUGC | 4883 | GCAUGAUC CUGAUGAGGCCGUUAGGCCGAA IAUAGAUU | 14223 |
| 3646 | AUCAGAUC A UGCUGGAC | 4884 | GUCCAGCA CUGAUGAGGCCGUUAGGCCGAA IAUCUGAU | 14224 |
| 3650 | GAUCAUGC U GGACUGCU | 4885 | AGCAGUCC CUGAUGAGGCCGUUAGGCCGAA ICAUGAUC | 14225 |
| 3655 | UGCUGGAC U GCUGGCAC | 4886 | GUGCCAGC CUGAUGAGGCCGUUAGGCCGAA IUCCAGCA | 14226 |
| 3658 | UGGACUGC U GGCACAGA | 4887 | UCUGUGCC CUGAUGAGGCCGUUAGGCCGAA ICAGUCCA | 14227 |
| 3662 | CUGCUGGC A CAGAGACC | 4888 | GGUCUCUG CUGAUGAGGCCGUUAGGCCGAA ICCAGCAG | 14228 |
| 3664 | GCUGGCAC A GAGACCCA | 4889 | UGGGUCUC CUGAUGAGGCCGUUAGGCCGAA IUGCCAGC | 14229 |
| 3670 | ACAGAGAC C CAAAAGAA | 4890 | UUCUUUUG CUGAUGAGGCCGUUAGGCCGAA IUCUCUGU | 14230 |
| 3671 | CAGAGACC C AAAAGAAA | 4891 | UUUCUUUU CUGAUGAGGCCGUUAGGCCGAA IGUCUCUG | 14231 |
| 3672 | AGAGACCC A AAAGAAAG | 4892 | CUUUCUUU CUGAUGAGGCCGUUAGGCCGAA IGGUCUCU | 14232 |
| 3683 | AGAAAGGC C AAGAUUUG | 4893 | CAAAUCUU CUGAUGAGGCCGUUAGGCCGAA ICCUUUCU | 14233 |
| 3684 | GAAAGGCC A AGAUUUGC | 4894 | GCAAAUCU CUGAUGAGGCCGUUAGGCCGAA IGCCUUUC | 14234 |
| 3693 | AGAUUUGC A GAACUUGU | 4895 | ACAAGUUC CUGAUGAGGCCGUUAGGCCGAA ICAAAUCU | 14235 |
| 3698 | UGCAGAAC U UGUGGAAA | 4896 | UUUCCACA CUGAUGAGGCCGUUAGGCCGAA IUUCUGCA | 14236 |
| 3710 | GGAAAAAC U AGGUGAUU | 4897 | AAUCACCU CUGAUGAGGCCGUUAGGCCGAA IUUUUUCC | 14237 |
| 3722 | UGAUUUGC U UCAAGCAA | 4898 | UUGCUUGA CUGAUGAGGCCGUUAGGCCGAA ICAAAUCA | 14238 |
| 3725 | UUUGCUUC A AGCAAAUG | 4899 | CAUUUGCU CUGAUGAGGCCGUUAGGCCGAA IAAGCAAA | 14239 |
| 3729 | CUUCAAGC A AAUGUACA | 4900 | UGUACAUU CUGAUGAGGCCGUUAGGCCGAA ICUUGAAG | 14240 |
| 3737 | AAAUGUAC A ACAGGAUG | 4901 | CAUCCUGU CUGAUGAGGCCGUUAGGCCGAA IUACAUUU | 14241 |
| 3740 | UGUACAAC A GGAUGGUA | 4902 | UACCAUCC CUGAUGAGGCCGUUAGGCCGAA IUUGUACA | 14242 |
| 3754 | GUAAAGAC U ACAUCCCA | 4903 | UGGGAUGU CUGAUGAGGCCGUUAGGCCGAA IUCUGUAC | 14243 |
| 3757 | AAGACUAC A UCCCAAUC | 4904 | GAUUGGGA CUGAUGAGGCCGUUAGGCCGAA IUAGUCUU | 14244 |
| 3760 | ACUACAUC C CAAUCAAU | 4905 | AUUGAUUG CUGAUGAGGCCGUUAGGCCGAA IAUGUAGU | 14245 |
| 3761 | CUACAUCC C AAUCAAUG | 4906 | CAUUGAUU CUGAUGAGGCCGUUAGGCCGAA IGAUGUAG | 14246 |
| 3762 | UACAUCCC A AUCAAUGC | 4907 | GCAUUGAU CUGAUGAGGCCGUUAGGCCGAA IGGAUGHA | 14247 |
| 3766 | UCCCAAUC A AUGCCAUA | 4908 | UAUGGCAU CUGAUGAGGCCGUUAGGCCGAA IAUUGGGA | 14248 |
| 3771 | AUCAAUGC C AUACUGAC | 4909 | GUCAGUAU CUGAUGAGGCCGUUAGGCCGAA ICAUUGAU | 14249 |
| 3772 | UCAAUGCC A UACUGACA | 4910 | UGUCAGUA CUGAUGAGGCCGUUAGGCCGAA IGCAUUGA | 14250 |
| 3776 | UGCCAUAC U GACAGGAA | 4911 | UUCCUGUC CUGAUGAGGCCGUUAGGCCGAA IUAUGGCA | 14251 |
| 3780 | AUACUGAC A GGAAAUAG | 4912 | CUAUUUCC CUGAUGAGGCCGUUAGGCCGAA IUCAGUAU | 14252 |
| 3798 | GGGUUUAC A UACUCAAC | 4913 | GUUGAGUA CUGAUGAGGCCGUUAGGCCGAA IUAAACCC | 14253 |
| 3802 | UUACAUAC U CAACUCCU | 4914 | AGGAGUUG CUGAUGAGGCCGUUAGGCCGAA IUAUGUAA | 14254 |
| 3804 | ACAUACUC A ACUCCUGC | 4915 | GCAGGAGU CUGAUGAGGCCGUUAGGCCGAA IAGUAUGU | 14255 |
| 3807 | UACUCAAC U CCUGCCUU | 4916 | AAGGCAGG CUGAUGAGGCCGUUAGGCCGAA IUUGAGUA | 14256 |
| 3809 | CUCAACUC C UGCCUUCU | 4917 | AGAAGGCA CUGAUGAGGCCGUUAGGCCGAA IAGUUCAG | 14257 |
| 3810 | UCAACUCC U CCCUUCUC | 4918 | GAGAAGGC CUGAUGAGGCCGUUAGGCCGAA IGAGUUGA | 14258 |
| 3813 | ACUCCUGC C UUCUCUGA | 4919 | UCAGAGAA CUGAUGAGGCCGUUAGGCCGAA ICAGGAGU | 14259 |
| 3814 | CUCCUGCC U UCUCUGAG | 4920 | CUCAGAGA CUGAUGAGGCCGUUAGGCCGAA ICCAGGAG | 14260 |
| 3817 | CUGCCUUC U CUGAGGAC | 4921 | GUCCUCAG CUGAUGAGGCCGUUAGGCCGAA IAAGGCAG | 14261 |
| 3819 | GCCUUCUC U GAGGACUU | 4922 | AAGUCCUC CUGAUGAGGCCGUUAGGCCGAA IAGAAGGC | 14262 |
| 3826 | CUGAGGAC U UCUUCAAG | 4923 | CUUGAAGA CUGAUGAGGCCGUUAGGCCGAA IUCCUCAG | 14263 |
| 3829 | AGGACUUC U UCAAGGAA | 4924 | UUCCUUGA CUGAUGAGGCCGUUAGGCCGAA IAAGUCCU | 14264 |
| 3832 | ACUUCUUC A AGGAAAGU | 4925 | ACUUUCCU CUGAUGAGGCCGUUAGGCCGAA IAAGAAGU | 14265 |
| 3846 | AGUADUUC A GCUCCGAA | 4926 | UUCGGAGC CUGAUGAGGCCGUUAGGCCGAA IAAAUACU | 14266 |
| 3849 | AUUUCAGC U CCGAAGUU | 4927 | AACUUCGG CUGAUGAGGCCGUUAGGCCGAA ICUGAAAU | 14267 |
| 3851 | UUCAGCUC C GAAGUUUA | 4928 | UAAACUUC CUGAUGAGGCCGUUAGGCCGAA IAGCUGAA | 14268 |
| 3864 | UUUAAUUC A GGAAGCUC | 4929 | GAGCUUCC CUGAUGAGGCCGUUAGGCCGAA IAAUUAAA | 14269 |
| 3871 | CAGGAAGC U CUGAUGAU | 4930 | AUCAUCAG CUGAUGAGGCCGUUAGGCCGAA ICUUCCUG | 14270 |
| 3873 | GGAAGCUC U GAUGAUGU | 4931 | ACAUCAUC CUGAUGAGGCCGUUAGGCCGAA IAGCUUCC | 14271 |
| 3883 | AUGAUGUC A GAUAUGUA | 4932 | UACAUAUC CUGAUGAGGCCGUUAGGCCGAA IACAUCAU | 14272 |
| 3897 | GUAAAUGC U UUCAAGUU | 4933 | AACUUGAA CUGAUGAGGCCGUUAGGCCGAA ICAUUUAC | 14273 |
| 3901 | AUGCUUUC A AGUUCAUG | 4934 | CAUGAACU CUGAUGAGGCCGUUAGGCCGAA IAAAGCAU | 14274 |
| 3907 | UCAAGUUC A UGAGCCUG | 4935 | CAGGCUCA CUGAUGAGGCCGUUAGGCCGAA IAACUUGA | 14275 |
| 3913 | UCAUGAGC C UGGAAAGA | 4936 | UCUUUCCA CUGAUGAGGCCGUUAGGCCGAA ICUCAUGA | 14276 |
| 3914 | CAUGAGCC U GGAAAGAA | 4937 | UUCUUUCC CUGAUGAGGCCGUUAGGCCGAA IGCUCAUG | 14277 |
| 3925 | AAAGAAUC A AAACCUUU | 4938 | AAAGGUUU CUGAUGAGGCCGUUAGGCCGAA IAUUCUUU | 14278 |
| 3930 | AUCAAAAC C UUUGAAGA | 4939 | UCUUCAAA CUGAUGAGGCCGUUAGGCCGAA IUUUUGAU | 14279 |
| 3931 | UCAAAACC U UUGAAGAA | 4940 | UUCUUCAA CUGAUGAGGCCGUUAGGCCGAA IGUUUUGA | 14280 |
| 3941 | UGAAGAAC U UUUACCGA | 4941 | UCGGUAAA CUGAUGAGGCCGUUAGGCCGAA IUUCUUCA | 14281 |
| 3947 | ACUUUUAC C GAAUGCCA | 4942 | UGGCAUUC CUGAUGAGGCCGUUAGGCCGAA IUAAAAGU | 14282 |
| 3954 | CCGAAUGC C ACCUCCAU | 4943 | AUGGAGGU CUGAUGAGGCCGUUAGGCCGAA ICAUUCGG | 14283 |
| 3955 | CGAAUGCC A CCUCCAUG | 4944 | CAUGGAGG CUGAUGAGGCCGUUAGGCCGAA IGCAUUCG | 14284 |
| 3957 | AAUGCCAC C UCCAUGUU | 4945 | AACAUGGA CUGAUGAGGCCGUUAGGCCGAA IUGGCAUU | 14285 |
| 3958 | AUGCCACC U CCAUGUUU | 4946 | AAACAUGG CUGAUGAGGCCGUUAGGCCGAA IGUGGCAU | 14286 |
| 3960 | GCCACCUC C AUGUUUGA | 4947 | UCAAACAU CUGAUGAGGCCGUUAGGCCGAA IAGGUGGC | 14287 |
| 3961 | CCACCUCC A UGUUUGAU | 4948 | AUCAAACA CUGAUGAGGCCGUUAGGCCGAA IGAGGUGG | 14288 |
| 3973 | UUGAUGAC U ACCAGGGC | 4949 | GCCCUGGU CUGAUGAGGCCGUUAGGCCGAA IUCAUCAA | 14289 |
| 3976 | AUGACUAC C AGGGCGAC | 4950 | GUCGCCCU CUGAUGAGGCCGUUAGGCCGAA IUAGUCAU | 14290 |
| 3977 | UGACUACC A GGGCGACA | 4951 | UGUCGCCC CUGAUGAGGCCGUUAGGCCGAA IGUAGUCA | 14291 |
| 3985 | AGGGCGAC A GCAGCACU | 4952 | AGUGCUGC CUGAUGAGGCCGUUAGGCCGAA IUCGCCCU | 14292 |
| 3988 | GCGACAGC A GCACUCUG | 4953 | CAGAGUGC CUGAUGAGGCCGUUAGGCCGAA ICUGUCGC | 14293 |
| 3991 | ACAGCAGC A CUCUGUUG | 4954 | CAACAGAG CUGAUGAGGCCGUUAGGCCGAA ICUGCUGU | 14294 |
| 3993 | AGCAGCAC U CUGUUGGC | 4955 | GCCAACAG CUGAUGAGGCCGUUAGGCCGAA IUGCUGCU | 14295 |
| 3995 | CAGCACUC U GUUGGCCU | 4956 | AGGCCAAC CUGAUGAGGCCGUUAGGCCGAA IAGUGCUG | 14296 |
| 4002 | CUGUUGGC C UCUCCCAU | 4957 | AUGGGAGA CUGAUGAGGCCGUUAGGCCGAA ICCAACAG | 14297 |
| 4003 | UGUUGGCC U CUCCCAUG | 4958 | CAUGGGAG CUGAUGAGGCCGUUAGGCCGAA IGCCAACA | 14298 |
| 4005 | UUGGCCUC U CCCAUGCU | 4959 | AGCAUGGG CUGAUGAGGCCGUUAGGCCGAA IAGGCCAA | 14299 |
| 4007 | GGCCUCUC C CAUGCUGA | 4960 | UCAGCAUG CUGAUGAGGCCGUUAGGCCGAA IAGAGGCC | 14300 |
| 4008 | GCCUCUCC C AUGCUGAA | 4961 | UUCAGCAU CUGAUGAGGCCGUUAGGCCGAA IGAGAGGC | 14301 |
| 4009 | CCUCUCCC A UGCUGAAG | 4962 | CUUCAGCA CUGAUGAGGCCGUUAGGCCGAA IGGAGAGG | 14302 |
| 4013 | UCCCAUGC U GAAGCGCU | 4963 | AGCGCUUC CUGAUGAGGCCGUUAGGCCGAA ICAUGGGA | 14303 |
| 4021 | UGAAGCGC U UCACCUGG | 4964 | CCAGGUGA CUGAUGAGGCCGUUAGGCCGAA ICGCUUCA | 14304 |
| 4024 | AGCGCUUC A CCUGGACU | 4965 | AGUCCAUG CUGAUGAGGCCGUUAGGCCGAA IAAGCGCU | 14305 |
| 4026 | CGCUUCAC C UGGACUGA | 4966 | UCAGUCCA CUGAUGAGGCCGUUAGGCCGAA IUGAAGCG | 14306 |
| 4027 | GCUUCACC U GGACUGAC | 4967 | GUCAGUCC CUGAUGAGGCCGUUAGGCCGAA IGUGAAGC | 14307 |
| 4032 | ACCUGGAC U GACAGCAA | 4968 | UUGCUGUC CUGAUGAGGCCGUUAGGCCGAA IUCCAGGU | 14308 |
| 4036 | GGACUGAC A GCAAACCC | 4969 | GGGUUUGC CUGAUGAGGCCGUUAGGCCGAA IUCAGUCC | 14309 |
| 4039 | CUGACAGC A AACCCAAG | 4970 | CUUGGGUU CUGAUGAGGCCGUUAGGCCGAA ICUGUCAG | 14310 |
| 4043 | CAGCAAAC C CAAGGCCU | 4971 | AGGCCUUG CUGAUGAGGCCGUUAGGCCGAA IUUUGCUG | 14311 |
| 4044 | AGCAAACC C AAGGCCUC | 4972 | GAGGCCUU CUGAUGAGGCCGUUAGGCCGAA IGUUUGCU | 14312 |
| 4045 | GCAAACCC A AGGCCUCG | 4973 | CGAGGCCU CUGAUGAGGCCGUUAGGCCGAA IGGUUUGC | 14313 |
| 4050 | CCCAAGGC C UCGCUCAA | 4974 | UUGAGCGA CUGAUGAGGCCGUUAGGCCGAA ICCUUGGG | 14314 |
| 4051 | CCAAGGCC U CGCUCAAG | 4975 | CUUGAGCG CUGAUGAGGCCGUUAGGCCGAA IGCCUUGG | 14315 |
| 4055 | GGCCUCGC U CAAGAUUG | 4976 | CAAUCUUG CUGAUGAGGCCGUUAGGCCGAA ICGAGGCC | 14316 |
| 4057 | CCUCGCUC A AGAUUGAC | 4977 | GUCAAUCU CUGAUGAGGCCGUUAGGCCGAA IAGCGAGG | 14317 |
| 4066 | AGAUUGAC U UGAGAGUA | 4978 | UACUCUCA CUGAUGAGGCCGUUAGGCCGAA IUCAAUCU | 14318 |
| 4077 | AGAGUAAC C AGUAAAAG | 4979 | CUUUUACU CUGAUGAGGCCGUUAGGCCGAA IUUACUCU | 14319 |
| 4078 | GAGUAACC A GUAAAAGU | 4980 | ACUUUUAC CUGAUGAGGCCGUUAGGCCGAA IGUUACUC | 14320 |
| 4100 | GUCGGGGC U GUCUGAUG | 4981 | CAUCAGAC CUGAUGAGGCCGUUAGGCCGAA ICCCCGAC | 14321 |
| 4104 | GGGCUGUC U GAUGUCAG | 4982 | CUGACAUC CUGAUGAGGCCGUUAGGCCGAA IACAGCCC | 14322 |
| 4111 | CUGAUGUC A GCAGGCCC | 4983 | GGGCCUGC CUGAUGAGGCCGUUAGGCCGAA IACAUCAG | 14323 |
| 4114 | AUGUCAGC A GGCCCAGU | 4984 | ACUGGGCC CUGAUGAGGCCGUUAGGCCGAA ICUGACAU | 14324 |
| 4118 | CAGCAGGC C CAGUUUCU | 4985 | AGAAACUG CUGAUGAGGCCGUUAGGCCGAA ICCUCCUG | 14325 |
| 4119 | AGCAGGCC C AGUUUCUG | 4986 | CAGAAACU CUGAUGAGGCCGUUAGGCCGAA IGCCUGCU | 14326 |
| 4120 | GCAGGCCC A GUUUCUGC | 4987 | GCAGAAAC CUGAUGAGGCCGUUAGGCCGAA IGGCCUGC | 14327 |
| 4126 | CCAGUUUC U GCCAUUCC | 4988 | GGAAUGGC CUGAUGAGGCCGUUAGGCCGAA IAAACUGG | 14328 |
| 4129 | GUUUCUGC C AUUCCAGC | 4989 | GCUGGAAU CUGAUGAGGCCGUUAGGCCGAA ICAGAAAC | 14329 |
| 4130 | UUUCUGCC A UUCCAGCU | 4990 | AGCUGGAA CUGAUGAGGCCGUUAGGCCGAA IGCAGAAA | 14330 |
| 4134 | UGCCAUUC C AGCUGUGG | 4991 | CCACAGCU CUGAUGAGGCCGUUAGGCCGAA IAAUGGCA | 14331 |
| 4135 | GCCAUUCC A GCUGUGGG | 4992 | CCCACAGC CUGAUGAGGCCGUUAGGCCGAA IGAAUGGC | 14332 |
| 4138 | AUUCCAGC U GUGGGCAC | 4993 | GUGCCCAC CUGAUGAGGCCGUUAGGCCGAA ICUGGAAU | 14333 |
| 4145 | CUGUGGGC A CGUCAGCG | 4994 | CGCUGACG CUGAUGAGGCCGUUAGGCCGAA ICCCACAG | 14334 |
| 4150 | GGCACGUC A GCGAAGGC | 4995 | GCCUUCGC CUGAUGAGGCCGUUAGGCCGAA IACGUGCC | 14335 |
| 4159 | GCGAAGGC A AGCGCAGG | 4996 | CCUGCGCU CUGAUGAGGCCGUUAGGCCGAA ICCUUCGC | 14336 |
| 4165 | GCAAGCGC A GGUUCACC | 4997 | GGUGAACC CUGAUGAGGCCGUUAGGCCGAA ICGCUUGC | 14337 |
| 4171 | GCAGGUUC A CCUACGAC | 4998 | GUCGUAGG CUGAUGAGGCCGUUAGGCCGAA IAACCUGC | 14338 |
| 4173 | AGGUUCAC C UACGACCA | 4999 | UGGUCGUA CUGAUGAGGCCGUUAGGCCGAA IUGAACCU | 14339 |
| 4174 | GGUUCACC U ACGACCAC | 5000 | GUGGUCGU CUGAUGAGGCCGUUAGGCCGAA IGUGAACC | 14340 |
| 4180 | CCUACGAC C ACGCUGAG | 5001 | CUCAGCGU CUGAUGAGGCCGUUAGGCCGAA IUCGUAGG | 14341 |
| 4181 | CUACGACC A CGCUGAGC | 5002 | GCUCAGCG CUGAUGAGGCCGUUAGGCCGAA IGUCCUAG | 14342 |
| 4185 | GACCACGC U GAGCUGGA | 5003 | UCCAGCUC CUGAUGAGGCCGUUAGGCCGAA ICGUGGUC | 14343 |
| 4190 | CGCUGAGC U GGAAAGGA | 5004 | UCCUUUCC CUGAUGAGGCCGUUAGGCCGAA ICUCAGCG | 14344 |
| 4210 | UCGCGUGC U GCUCCCCG | 5005 | CGGGGAGC CUGAUGAGGCCGUUAGGCCGAA ICACGCGA | 14345 |
| 4213 | CGUGCUGC U CCCCGCCC | 5006 | GGGCGGGG CUGAUGAGGCCGUUAGGCCGAA ICAGCACG | 14346 |
| 4215 | UGCUGCUC C CCGCCCCC | 5007 | GGGGGCGG CUGAUGAGGCCGUUAGGCCGAA IAUCAUCA | 14347 |
| 4216 | GCUGCUCC C CGCCCCCA | 5008 | UGGGGGCG CUGAUGAGGCCGUUAGGCCGAA IGAGCAGC | 14348 |
| 4217 | CUGCUCCC C GCCCCCAG | 5009 | CUGGGGGC CUGAUGAGGCCGUUAGGCCGAA IGGAGCAG | 14349 |
| 4220 | CUCCCCGC C CCCAGACU | 5010 | AGUCUGGG CUGAUGAGGCCGUUAGGCCGAA ICGGGGAG | 14350 |
| 4221 | UCCCCGCC C CCAGACUA | 5011 | UAGUCUGG CUGAUGAGGCCGUUAGGCCGAA IGCGGGGA | 14351 |
| 4222 | CCCCGCCC C CAGACUAC | 5012 | GUAGUCUG CUGAUGAGGCCGUUAGGCCGAA IGGCGGGG | 14352 |
| 4223 | CCCGCCCC C AGACUACA | 5013 | UGUAGUCU CUGAUGAGGCCGUUAGGCCGAA IGGGCGGG | 14353 |
| 4224 | CCGCCCCC A GACUACAA | 5014 | UUGUAGUC CUGAUGAGGCCGUUAGGCCGAA IGGGGCGG | 14354 |
| 4228 | CCCCAGAC U ACAACUCG | 5015 | CGAGUUGU CUGAUGAGGCCGUUAGGCCGAA IUCUGGUG | 14355 |
| 4231 | CAGACUAC A ACUCGGUG | 5016 | CACCGAGU CUGAUGAGGCCGUUAGGCCGAA IUAGUCUG | 14356 |
| 4234 | ACUACAAC U CGGUGGUC | 5017 | GACCACCG CUGAUGAGGCCGUUAGGCCGAA IUUGUAGU | 14357 |
| 4243 | CGGUGGUC C UGUACUCC | 5018 | GGAGUACA CUGAUGAGGCCGUUAGGCCGAA IACCACCG | 14358 |
| 4244 | GGUGGUCC U GUACUCCA | 5019 | UGGAGUAC CUGAUGAGGCCGUUAGGCCGAA IGACCACC | 14359 |
| 4249 | UCCUGUAC U CCACCCCA | 5020 | UGGUGUGG CUGAUGAGGCCGUUAGGCCGAA IUACAGGA | 14360 |
| 4251 | CUGUACUC C ACCCCACC | 5021 | GGUGGGGU CUGAUGAGGCCGUUAGGCCGAA IAGUACAG | 14361 |
| 4252 | UGUACUCC A CCCCACCC | 5022 | GGGUGGGG CUGAUGAGGCCGUUAGGCCGAA IGAGUACA | 14362 |
| 4254 | UACUCCAC C CCACCCAU | 5023 | AUGGGUGG CUGAUGAGGCCGUUAGGCCGAA IUGGAGHA | 14363 |
| 4255 | ACUCCACC C CACCCAUC | 5024 | GAUGGGUG CUGAUGAGGCCGUUAGGCCGAA IGUGGAGU | 14364 |
| 4256 | CUCCACCC C ACCCAUCU | 5025 | AGAUGGGU CUGAUGAGGCCGUUAGGCCGAA IGGUGGAG | 14365 |
| 4257 | UCCACCCC A CCCAUCUA | 5026 | UAGAUGGG CUGAUGAGGCCGUUAGGCCGAA IGGGUGGA | 14366 |
| 4259 | CACCCCAC C CAUCUAGA | 5027 | UCUAGAUG CUGAUGAGGCCGUUAGGCCGAA IUGGGGUG | 14367 |
| 4260 | ACCCCACC C AUCUAGAG | 5028 | CUCUAGAU CUGAUGAGGCCGUUAGGCCGAA IGUGGGGU | 14368 |
| 4261 | CCCCACCC A UCUAGAGU | 5029 | ACUCUAGA CUGAUGAGGCCGUUAGGCCGAA IGGUGGGG | 14369 |
| 4264 | CACCCAUC U AGAGUUUG | 5030 | CAAACUCU CUGAUGAGGCCGUUAGGCCGAA IAUGGGUG | 14370 |
| 4275 | AGUUUGAC A CGAAGCCU | 5031 | AGGCUUCG CUGAUGAGGCCGUUAGGCCGAA IUCAAACU | 14371 |
| 4282 | CACGAAGC C UUAUUUCU | 5032 | AGAAAUAA CUGAUGAGGCCGUUAGGCCGAA ICUUCGUG | 14372 |
| 4283 | ACGAAGCC U UAUUUCUA | 5033 | UAGAAAUA CUGAUGAGGCCGUUAGGCCGAA IGCUUCGU | 14373 |
| 4290 | CUUAUUUC U AGAAGCAC | 5034 | GUGCUUCU CUGAUGAGGCCGUUAGGCCGAA IAAAUAAG | 14374 |
| 4297 | CUAGAAGC A CAUGUGUA | 5035 | UACACAUG CUGAUGAGGCCGUUAGGCCGAA ICUUCUAG | 14375 |
| 4299 | AGAAGCAC A UGUGUAUU | 5036 | AAUACACA CUGAUGAGGCCGUUAGGCCGAA IUGCUUCU | 14376 |
| 4313 | AUUUAUAC C CCCAGGAA | 5037 | UUCCUGGG CUGAUGAGGCCGUUAGGCCGAA IUAUAAAU | 14377 |
| 4314 | UUUAUACC C CCAGGAAA | 5038 | UUUCCUGG CUGAUGAGGCCGUUAGGCCGAA IGUAUAAA | 14378 |
| 4315 | UUAUACCC C CAGGAAAC | 5039 | GUUUCCUG CUGAUGAGGCCGUUAGGCCGAA IGGUAUAA | 14379 |
| 4316 | UAUACCCC C AGGAAACU | 5040 | AGUUUCCU CUGAUGAGGCCGUUAGGCCGAA IGGGUAUA | 14380 |
| 4317 | AUACCCCC A GGAAACUA | 5041 | UAGUUUCC CUGAUGAGGCCGUUAGGCCGAA IGGGGUAU | 14381 |
| 4324 | CAGGAAAC U AGCUUUUG | 5042 | CAAAAGCU CUGAUGAGGCCGUUAGGCCGAA IUUUCCUG | 14382 |
| 4328 | AAACUAGC U UUUGCCAG | 5043 | CUGGCAAA CUGAUGAGGCCGUUAGGCCGAA ICUAGUUU | 14383 |
| 4334 | GCUUUUGC C AGUAUUAU | 5044 | AUAAUACU CUGAUGAGGCCGUUAGGCCGAA ICAAAAGC | 14384 |
| 4335 | CUUUUGCC A GUAUUAUG | 5045 | CAUAAUAC CUGAUGAGGCCGUUAGGCCGAA IGCAAAAG | 14385 |
| 4345 | UAUUAUGC A UAUAUAAG | 5046 | CUUAUAUA CUGAUGAGGCCGUUAGGCCGAA ICAUAAUA | 14386 |
| 4359 | AAGUUUAC A CCUUUAUC | 5047 | GAUAAAGG CUGAUGAGGCCGUUAGGCCGAA IUAAACUU | 14387 |
| 4361 | GUUUACAC C UUUAUCUU | 5048 | AAGAUAAA CUGAUGAGGCCGUUAGGCCGAA IUGUAAAC | 14388 |
| 4362 | UUUACACC U UUAUCUUU | 5049 | AAAGAUAA CUGAUGAGGCCGUUAGGCCGAA IGUGUAAA | 14389 |
| 4368 | CCUUUAUC U UUCCAUGG | 5050 | CCAUGGAA CUGAUGAGGCCGUUAGGCCGAA IAUAAAGG | 14390 |
| 4372 | UAUCUUUC C AUGGGAGC | 5051 | GCUCCCAU CUGAUGAGGCCGUUAGGCCGAA IAAAGAUA | 14391 |
| 4373 | AUCUUUCC A UGGGAGCC | 5052 | GGCUCCCA CUGAUGAGGCCGUUAGGCCGAA IGAAAGAU | 14392 |
| 4381 | AUGGGAGC C AGCUGCUU | 5053 | AAGCAGCU CUGAUGAGGCCGUUAGGCCGAA ICUCCCAU | 14393 |
| 4382 | UGGGAGCC A GCUGCUUU | 5054 | AAAGCAGC CUGAUGAGGCCGUUAGGCCGAA IGCUCCCA | 14394 |
| 4385 | GAGCCAGC U GCUUUUUG | 5055 | CAAAAAGC CUGAUGAGGCCGUUAGGCCGAA ICUGGCUC | 14395 |
| 4388 | CCAGCUGC U UUUUGUGA | 5056 | UCACAAAA CUGAUGAGGCCGUUAGGCCGAA ICAGCUGG | 14396 |
| 4412 | AAUAGUGC U UUUUUUUU | 5057 | AAAAAAAA CUGAUGAGGCCGUUAGGCCGAA ICACUAUU | 14397 |
| 4426 | UUUUUGAC U AACAAGAA | 5058 | UUCUUGUU CUGAUGAGGCCGUUAGGCCGAA IUCAAAAA | 14398 |
| 4430 | UGACUAAC A AGAAUGUA | 5059 | UACAUUCU CUGAUGAGGCCGUUAGGCCGAA IUUAGUCA | 14399 |
| 4441 | AAUGUAAC U CCAGAUAG | 5060 | CUAUCUGG CUGAUGAGGCCGUUAGGCCGAA IUUACAUU | 14400 |
| 4443 | UGUAACUC C AGAUAGAG | 5061 | CUCUAUCU CUGAUGAGGCCGUUAGGCCGAA IAGUUACA | 14401 |
| 4444 | GUAACUCC A GAUAGAGA | 5062 | UCUCUAUC CUGAUGAGGCCGUUAGGCCGAA IGAGUUAC | 14402 |
| 4462 | AUAGUGAC A AGUGAAGA | 5063 | UCUUCACU CUGAUGAGGCCGUUAGGCCGAA IUCACUAU | 14403 |
| 4473 | UGAAGAAC A CUACUGCU | 5064 | AGCAGUAG CUGAUGAGGCCGUUAGGCCGAA IUUCUUCA | 14404 |
| 4475 | AAGAACAC U ACUGCUAA | 5065 | UUAGCAGU CUGAUGAGGCCGUUAGGCCGAA IUGUUCUU | 14405 |
| 4478 | AACACUAC U GCUAAAUC | 5066 | GAUUUAGC CUGAUGAGGCCGUUAGGCCGAA IUAGUGUU | 14406 |
| 4481 | ACUACUGC U AAAUCCUC | 5067 | GAGGAUUU CUGAUGAGGCCGUUAGGCCGAA ICAGUAGU | 14407 |
| 4487 | GCUAAAUC C UCAUGUUA | 5068 | UAACAUGA CUGAUGAGGCCGUUAGGCCGAA IAUUUAGC | 14408 |
| 4488 | CUAAAUCC U CAUGUUAC | 5069 | GUAACAUG CUGAUGAGGCCGUUAGGCCGAA IGAUUUAG | 14409 |
| 4490 | AAAUCCUC A UGUUACUC | 5070 | GAGUAACA CUGAUGAGGCCGUUAGGCCGAA IAGGAUUU | 14410 |
| 4497 | CAUGUUAC U CAGUGUUA | 5071 | UAACACUG CUGAUGAGGCCGUUAGGCCGAA IUAACAUG | 14411 |
| 4499 | UGUUACUC A GUGUUAGA | 5072 | UCUAACAC CUGAUGAGGCCGUUAGGCCGAA IAGUAACA | 14412 |
| 4514 | GAGAAAUC C UUCCUAAA | 5073 | UUUAGGAA CUGAUGAGGCCGUUAGGCCGAA IAUUUCUC | 14413 |
| 4515 | AGAAAUCC U UCCUAAAC | 5074 | GUUUAGGA CUGAUGAGGCCGUUAGGCCGAA IGAUUUCU | 14414 |
| 4518 | AAUCCUUC C UAAACCCA | 5075 | UGGGUUUA CUGAUGAGGCCGUUAGGCCGAA IAAGGAUU | 14415 |
| 4519 | AUCCUUCC U AAACCCAA | 5076 | UUGGGUUU CUGAUGAGGCCGUUAGGCCGAA IGAAGGAU | 14416 |
| 4524 | UCCUAAAC C CAAUGACU | 5077 | AGUCAUUG CUGAUGAGGCCGUUAGGCCGAA IUUUAGGA | 14417 |
| 4525 | CCUAAACC C AAUGACUU | 5078 | AAGUCAUU CUGAUGAGGCCGUUAGGCCGAA IGUUUAGG | 14418 |
| 4526 | CUAAACCC A AUGACUUC | 5079 | GAAGUCAU CUGAUGAGGCCGUUAGGCCGAA IGGUUUAG | 14419 |
| 4532 | CCAAUGAC U UCCCUGCU | 5080 | AGCAGGGA CUGAUGAGGCCGUUAGGCCGAA IUCAUUGG | 14420 |
| 4535 | AUGACUUC C CUGCUCCA | 5081 | UGGAGCAG CUGAUGAGGCCGUUAGGCCGAA IAAGUCAU | 14421 |
| 4536 | UGACUUCC C UGCUCCAA | 5082 | UUGGAGCA CUGAUGAGGCCGUUAGGCCGAA IGAAGUCA | 14422 |
| 4537 | GACUUCCC U GCUCCAAC | 5083 | GUUCGAGC CUGAUGAGGCCGUUAGGCCGAA IGGAAGUC | 14423 |
| 4540 | UUCCCUGC U CCAACCCC | 5084 | GGGGUUGG CUGAUGAGGCCGUUAGGCCGAA ICAGGGAA | 14424 |
| 4542 | CCCUGCUC C AACCCCCG | 5085 | CGGGGGUU CUGAUGAGGCCGUUAGGCCGAA IAGCAGGG | 14425 |
| 4553 | CCUGCUCC A ACCCCCGC | 5086 | GCGGGGGU CUGAUGAGGCCGUUAGGCCGAA IGAGCAGG | 14426 |
| 4546 | GCUCCAAC C CCCGCCAC | 5087 | GUGGCGGG CUGAUGAGGCCGUUAGGCCGAA IUUGGAGC | 14427 |
| 4547 | CUCCAACC C CCGCCACC | 5088 | GGUGGCGG CUGAUGAGGCCGUUAGGCCGAA IGUUGGAG | 14428 |
| 4548 | UCCAACCC C CGCCACCU | 5089 | AGGUGGCG CUGAUGAGGCCGUUAGGCCGAA IGGUUGGA | 14429 |
| 4549 | CCAACCCC C GCCACCUC | 5090 | GAGGUGGC CUGAUGAGGCCGUUAGGCCGAA IGGGUUGG | 14430 |
| 4552 | ACCCCCGC C ACCUCAGG | 5091 | CCUGAGGU CUGAUGAGGCCGUUAGGCCGAA ICGGGGGU | 14431 |
| 4553 | CCCCCGCC A CCUCAGGG | 5092 | CCCUGAGG CUGAUGAGGCCGUUAGGCCGAA IGCGGGGG | 14432 |
| 4555 | CCCGCCAC C UCAGGGCA | 5093 | UGCCCUGA CUGAUGAGGCCGUUAGGCCGAA IUGGCGGG | 14433 |
| 4556 | CCGCCACC U CAGGGCAC | 5094 | GUGCCCUG CUGAUGAGGCCGUUAGGCCGAA IGUGGCGG | 14434 |
| 4558 | GCCACCUC A GGGCACGC | 5095 | GCGUGCCC CUGAUGAGGCCGUUAGGCCGAA IAGGUGGC | 14435 |
| 4563 | CUCAGGGC A CGCAGGAC | 5096 | GUCCUGCG CUGAUGAGGCCGUUAGGCCGAA ICCCUGAG | 14436 |
| 4567 | GGGCACGC A GGACCAGU | 5097 | ACUGGUCC CUGAUGAGGCCGUUAGGCCGAA ICGUGCCC | 14437 |
| 4572 | CGCAGGAC C AGUUUGAU | 5098 | AUCAAACU CUGAUGAGGCCGUUAGGCCGAA IUCCUGCG | 14438 |
| 4573 | GCAGGACC A GUUUGAUU | 5099 | AAUCAAAC CUGAUGAGGCCGUUAGGCCGAA IGUCCUGC | 14439 |
| 4589 | UGAGGAGC U GCACUGAU | 5100 | AUCAGUGC CUGAUGAGGCCGUUAGGCCGAA ICUCCUCA | 14440 |
| 4592 | GGAGCUGC A CUGAUCAC | 5101 | GUGAUCAG CUGAUGAGGCCGUUAGGCCGAA ICAGCUCC | 14441 |
| 4594 | AGCUGCAC U GAUCACCC | 5102 | GGGUGAUC CUGAUGAGGCCGUUAGGCCGAA IUGCAGCU | 14442 |
| 4599 | CACUGAUC A CCCAAUGC | 5103 | GCAUUGGG CUGAUGAGGCCGUUAGGCCGAA IAUCAGUG | 14443 |
| 4601 | CUGAUCAC C CAAUGCAU | 5104 | AUGCAUUG CUGAUGAGGCCGUUAGGCCGAA IGUAUCAG | 14444 |
| 4602 | UGAUCACC C AAUGCAUC | 5105 | GAUGCAUU CUGAUGAGGCCGUUAGGCCGAA IGUGAUCA | 14445 |
| 4603 | GAUCACCC A AUGCAUCA | 5106 | UGAUGCAU CUGAUGAGGCCGUUAGGCCGAA IGGUGAUC | 14446 |
| 4608 | CCCAAUGC A UCACGUAC | 5107 | GUACGUGA CUGAUGAGGCCGUUAGGCCGAA ICAUUGGG | 14447 |
| 4611 | AAUGCAUC A CGUACCCC | 5108 | GGGGUACG CUGAUGAGGCCGUUAGGCCGAA IAUGCAUU | 14448 |
| 4617 | UCACGUAC C CCACUGGG | 5109 | CCCAGUGG CUGAUGAGGCCGUUAGGCCGAA IUACGUGA | 14449 |
| 4618 | CACGUACC C CACUGGGC | 5110 | GCCCAGUG CUGAUGAGGCCGUUAGGCCGAA IGUACGUG | 14450 |
| 4619 | ACGUACCC C ACUGGGCC | 5111 | GGCCCAGU CUGAUGAGGCCGUUAGGCCGAA IGGUACGU | 14451 |
| 4620 | CGUACCCC A CUGGGCCA | 5112 | UGGCCCAG CUGAUGAGGCCGUUAGGCCGAA IGGGUACG | 14452 |
| 4622 | UACCCCAC U GGGCCAGC | 5113 | GCUGGCCC CUGAUGAGGCCGUUAGGCCGAA IUGGGGUA | 14453 |
| 4627 | CACUGGGC C AGCCCUGC | 5114 | GCAGGGCU CUGAUGAGGCCGUUAGGCCGAA ICCCAGUG | 14454 |
| 4628 | ACUGGGCC A GCCCUGCA | 5115 | UGCAGGGC CUGAUGAGGCCGUUAGGCCGAA IGCCCAGU | 14455 |
| 4631 | GGGCCAGC C CUGCAGCC | 5116 | GGCUGCAG CUGAUGAGGCCGUUAGGCCGAA ICUGGCCC | 14456 |
| 4632 | GGCCAGCC C UGCAGCCC | 5117 | GGGCUGCA CUGAUGAGGCCGUUAGGCCGAA IGCUGGCC | 14457 |
| 4633 | GCCAGCCC U GCAGCCCA | 5118 | UGGGCUGC CUGAUGAGGCCGUUAGGCCGAA IGGCUGGC | 14458 |
| 4636 | AGCCCUGC A GCCCAAAA | 5119 | UUUUGGGC CUGAUGAGGCCGUUAGGCCGAA ICAGGGCU | 14459 |
| 4639 | CCUGCAGC C CAAAACCC | 5120 | GGGUUUUG CUGAUGAGGCCGUUAGGCCGAA ICUGCAGG | 14460 |
| 4640 | CUGCAGCC C AAAACCCA | 5121 | UGGCUUUU CUGAUGAGGCCGUUAGGCCGAA IGCUGCAG | 14461 |
| 4641 | UGCAGCCC A AAACCCAG | 5122 | CUGGGUUU CUGAUGAGGCCGUUAGGCCGAA IGGCUGCA | 14462 |
| 4646 | CCCAAAAC C CAGCGCAA | 5123 | UUGCCCUG CUGAUGAGGCCGUUAGGCCGAA IUUUUGGC | 14463 |
| 4647 | CCAAAACC C AGGGCAAC | 5124 | GUUGCCCU CUGAUGAGGCCGUUAGGCCGAA IGUUUUGG | 14464 |
| 4648 | CAAAACCC A GGGCAACA | 5125 | UGUUGCCC CUGAUGAGGCCGUUAGGCCGAA IGGUUUUG | 14465 |
| 4653 | CCCAGGGC A ACAAGCCC | 5126 | GGGCUUGU CUGAUGAGGCCGUUAGGCCGAA ICCCUGCG | 14466 |
| 4656 | AGGCCAAC A AGCCCGUU | 5127 | AACGGGCU CUGAUGAGGCCGUUAGGCCGAA IUUGCCCU | 14467 |
| 4660 | CAACAAGC C CGUUAGCC | 5128 | GGCUAACG CUGAUGAGGCCGUUAGGCCGAA ICUUGUUG | 14468 |
| 4661 | AACAAGCC C GUUAGCCC | 5129 | GGGCUAAC CUGAUGAGGCCGUUAGGCCGAA IGCUUGUU | 14469 |
| 4668 | CCGUUAGC C CCAGGGGA | 5130 | UCCCCUGG CUGAUGAGGCCGUUAGGCCGAA ICUAACGG | 14470 |
| 4669 | CGUUAGCC C CAGGGGAU | 5131 | AUCCCCUG CUGAUGAGGCCGUUACGCCGAA IGCUAACG | 14471 |
| 4670 | GUUAGCCC C AGGGGAUC | 5132 | GAUCCCCU CUGAUGAGGCCGUUAGGCCGAA IGGCUAAC | 14472 |
| 4671 | UUAGCCCC A GGGCAUCA | 5133 | UGAUCCCC CUGAUGAGGCCGUUAGGCCGAA IGGGCUAA | 14473 |
| 4679 | AGGGCAUC A CUGGCUGG | 5134 | CCAGCCAG CUGAUGAGGCCGUUAGGCCGAA IAUCCCCU | 14474 |
| 4681 | GGGAUCAC U GGCUGGCC | 5135 | GGCCAGCC CUGAUGAGGCCGUUAGGCCGAA IUGAUCCC | 14475 |
| 4685 | UCACUGGC U GGCCUGAG | 5136 | CUCACGCC CUGAUGAGGCCGUUAGGCCGAA ICCAGUCA | 14476 |
| 4689 | UGGCUGGC C UGACCAAC | 5137 | GUUGCUCA CUGAUGAGGCCGUUAGGCCGAA ICCAGCCA | 14477 |
| 4690 | GGCUGGCC U GAGCAACA | 5138 | UGUUGCUC CUGAUGAGGCCGUUAGGCCGAA IGCCAGCC | 14478 |
| 4695 | GCCUGAGC A ACAUCUCG | 5139 | CGAGAUGU CUGAUGAGGCCGUUAGGCCGAA ICUCAGGC | 14479 |
| 4698 | UGAGCPAC A UCUCGGGA | 5140 | UCCCGAGA CUGAUGAGGCCGUUAGGCCGAA IUUGCUCA | 14480 |
| 4701 | GCAACAUC U CGGGACUC | 5141 | GACUCCCG CUGAUGAGGCCGUUAGGCCGAA IAUGUUGC | 14481 |
| 4710 | CGGGACUC C UCUAGCAG | 5142 | CUGCUAGA CUGAUGAGGCCGUUAGGCCGAA IACUCCCG | 14482 |
| 4711 | GGGAGUCC U CUAGCAGG | 5143 | CCUGCUAG CUGAUGAGGCCGUUAGGCCGAA IGACUCCC | 14483 |
| 4713 | GAGUCCUC U AGCAGGCC | 5144 | GGCCUGCU CUGAUGAGGCCGUUAGGCCGAA IAGGACUC | 14484 |
| 4717 | CCUCUAGC A GGCCUAAG | 5145 | CUUAGGCC CUGAUGAGGCCGUUAGGCCGAA ICUAGAUG | 14485 |
| 4721 | UAGCAGGC C UAAGACAU | 5146 | AUGUCUUA CUGAUGAGGCCGUUAGGCCGAA ICCUGCUA | 14486 |
| 4722 | AGCAGGCC U AAGACAUG | 5147 | CAUGUCUU CUGAUGAGGCCGUUAGGCCGAA IGCCUGCU | 14487 |
| 4728 | CCUAAGAC A UGUGAGGA | 5148 | UCCUCACA CUGAUGAGGCCGUUAGGCCGAA IUCUUAGG | 14488 |
| 4754 | AAAAAAGC A AAAAGCAA | 5149 | UUGCUUUU CUGAUGAGGCCGUUAGGCCGAA ICUUUUUU | 14489 |
| 4761 | CAAAAAGC A AGGGAGAA | 5150 | UUCUCCCU CUGAUGAGGCCGUUAGGCCGAA ICUUUUUG | 14490 |
| 4779 | AGAGAAAC C GGGAGAAG | 5151 | CUUCUCCC CUGAUGAGGCCGUUAGGCCGAA IUUUCUCU | 14491 |
| 4790 | GAGAAGGC A UGAGAAAG | 5152 | CUUUCUCA CUGAUGAGGCCGUUAGGCCGAA ICCUUCUC | 14492 |
| 4811 | UGAGACGC A CCAUGUGG | 5153 | CCACAUGG CUGAUGAGGCCGUUAGGCCGAA ICCUCUCA | 14493 |
| 4813 | AGACGCAC C AUGUGGGC | 5154 | GCCCACAU CUGAUGAGGCCGUUAGGCCGAA IUGCGUCU | 14494 |
| 4814 | GACGCACC A UGUGGGCA | 5155 | UGCCCACA CUGAUGAGGCCGUUAGGCCGAA IGUGCGUC | 14495 |
| 4822 | AUGUGGGC A CGGAGGGG | 5156 | CCCCUCCG CUGAUGAGGCCGUUAGGCCGAA ICCCACAU | 14496 |
| 4839 | GACGGGGC U CAGCAAUG | 5157 | CAUUGCUG CUGAUGAGGCCGUUAGGCCGAA ICCCCGUC | 14497 |
| 4841 | CGGGGCUC A GCAAUGCC | 5158 | GGCAUUGC CUGAUGAGGCCGUUAGGCCGAA IAGCCCCG | 14498 |
| 4844 | GGCUCAGC A AUGCCAUU | 5159 | AAUGGCAU CUGAUGAGGCCGUUAGGCCGAA ICUGAGCC | 14499 |
| 4849 | AGCAAUGC C AUUUCAGU | 5160 | ACUGAAAU CUGAUGAGGCCGUUAGGCCGAA ICAUUGCU | 14500 |
| 4850 | GCAAUGCC A UUUCAGUG | 5161 | CACUGAAA CUGAUGAGGCCGUUAGGCCGAA IGCAUUGC | 14501 |
| 4855 | GCCAUUUC A GUGGCUUC | 5162 | GAAGCCAC CUGAUGAGGCCGUUAGGCCGAA IAAAUGGC | 14502 |
| 4861 | UCAGUGGC U UCCCAGCU | 5163 | AGCUGGGA CUGAUGAGGCCGUUAGGCCGAA ICCACUGA | 14503 |
| 4864 | GUGGCUUC C CAGCUCUG | 5164 | CAGAGCUG CUGAUGAGGCCGUUAGGCCGAA IAAGCCAC | 14504 |
| 4865 | UGGCUUCC C AGCUCUGA | 5165 | UCAGAGCU CUGAUGAGGCCGUUAGGCCGAA IGAAGCCA | 14505 |
| 4866 | GGCUUCCC A GCUCUGAC | 5166 | GUCAGAGC CUGAUGAGGCCGUUAGGCCGAA IGGAAGCC | 14506 |
| 4869 | UUCCCAGC U CUGACCCU | 5167 | AGGGUCAG CUGAUGAGGCCGUUAGGCCGAA ICUGGGAA | 14507 |
| 4871 | CCCAGCUC U GACCCUUC | 5168 | GAAGGGUC CUGAUGAGGCCGUUAGGCCGAA IAGCUGGG | 14508 |
| 4875 | GCUCUGAC C CUUCUACA | 5169 | UGUAGAAG CUGAUGAGGCCGUUAGGCCGAA IUCAGAGC | 14509 |
| 4876 | CUCUGACC C UUCUACAU | 5170 | AUGUAGAA CUGAUGAGGCCGUUAGGCCGAA IGUCAGAG | 14510 |
| 4877 | UCUGACCC U UCUACAUU | 5171 | AAUGUAGA CUGAUGAGGCCGUUAGGCCGAA IGGUCAGA | 14511 |
| 4880 | GACCCUUC U ACAUUUGA | 5172 | UCAAAUGU CUGAUGAGGCCGUUAGGCCGAA IAAGGCUC | 14512 |
| 4883 | CCUUCUAC A UUUGAGGG | 5173 | CCCUCAAA CUGAUGAGGCCGUUAGGCCGAA IUAGAAGG | 14513 |
| 4893 | UUGAGGGC C CACCCAGG | 5174 | CCUGGCUG CUGAUGAGGCCGUUAGGCCGAA ICCCUCAA | 14514 |
| 4894 | UGAGGGCC C AUCCAGGA | 5175 | UCCUGGCU CUGAUGAGGCCGUUAGGCCGAA IGCCCUCA | 14515 |
| 4895 | GAGGGCCC A GCCAGGAG | 5176 | CUCCUGGC CUGAUGAGGCCGUUAGGCCGAA IGGCCCUC | 14516 |
| 4898 | GGCCCAGC C AGGAGCAG | 5177 | CUGCUCCU CUGAUGAGGCCGUUAGGCCGAA ICUGGGCC | 14517 |
| 4899 | GCCCAGCC A GGAGCAGA | 5178 | UCUGCUCC CUGAUGAGGCCGUUAGGCCGAA IGCUGGGC | 14518 |
| 4905 | CCAGGAGC A GAUGGACA | 5179 | UGUCCAUC CUGAUGAGGCCGUUAGGCCGAA ICUCCUGG | 14519 |
| 4913 | AGAUGGAC A GGGAUGAG | 5180 | CUCAUCGC CUGAUGAGGCCGUUAGGCCGAA IUCCAUCU | 14520 |
| 4927 | GAGGGGAC A UUUUCUGG | 5181 | CCAGAAAA CUGAUGAGGCCGUUAGGCCGAA IUCCCCUC | 14521 |
| 4933 | ACAUUUUC U GUAGUCUG | 5182 | CAGAAUCC CUGAUGAGGCCGUUAGGCCGAA IAAAAUGU | 14522 |
| 4940 | CUGGAUUC U GGGAGGCA | 5183 | UGCCUCCC CUGAUGAGGCCGUUAGGCCGAA IAAUCCAG | 14523 |
| 4948 | UGGGAGGC A AGAAAAGG | 5184 | CCUUUUCU CUGAUGAGGCCGUUAGGCCGAA ICCUCCCA | 14524 |
| 4959 | AAAAGGAC A AAUAUCUU | 5185 | AAGAUAUU CUGAUGAGGCCGUUAGGCCGAA IUCCUUUU | 14525 |
| 4966 | CAAAUAUC U UUUUUGGA | 5186 | UCCAAAAA CUGAUGAGGCCGUUAGGCCGAA IAUAUUUG | 14526 |
| 4977 | UUUGGAAC U AAAGCAAA | 5187 | UUUGCUUU CUGAUGAGGCCGUUAGGCCGAA IUUCCAAA | 14527 |
| 4983 | ACUAAAGC A AAUUUUAG | 5188 | CUAAAAUU CUGAUGAGGCCGUUAGGCCGAA ICUUUAGU | 14528 |
| 4994 | UUUUAGAC C UUUACCUA | 5189 | UAGGUAAA CUGAUGAGGCCGUUAGGCCGAA IUCUAAAA | 14529 |
| 4995 | UUUAGACC U UUACCUAU | 5190 | AUAGGUAA CUGAUGAGGCCGUUAGGCCGAA IGUCUAAA | 14530 |
| 5000 | ACCUUUAC C UAUGGAAG | 5191 | CUUCCAUA CUGAUGAGGCCGUUAGGCCGAA IUAAAGGU | 14531 |
| 5001 | CCUUUACC U AUGGAAGU | 5192 | ACUUCCAU CUGAUGAGGCCGUUAGGCCGAA IGUAAAGG | 14532 |
| 5015 | AGUGGUUC U AUGUCCAU | 5193 | AUGGACAU CUGAUGAGGCCGUUAGGCCGAA IAACCACU | 14533 |
| 5021 | UCUAUGUC C AUUCUCAU | 5194 | AUGAGAAU CUGAUGAGGCCGUUAGGCCGAA IACAUAGA | 14534 |
| 5022 | CUAUGUCC A UUCUCAUU | 5195 | AAUGAGAA CUGAUGAGGCCGUUAGGCCGAA IGACAUAG | 14535 |
| 5026 | GUCCAUUC U CAUUCCUG | 5196 | CACGAAUG CUGAUGAGGCCGUUAGGCCGAA IAAUGGAC | 14536 |
| 5028 | CCAUUCUC A UUCGUGGC | 5197 | GCCACGAA CUGAUGAGGCCGUUAGGCCGAA IAGAAUGG | 14537 |
| 5037 | UUCGUGGC A UGUUUUGA | 5198 | UCAAAACA CUGAUGAGGCCGUUAGGCCGAA ICCACGAA | 14538 |
| 5054 | UUUGUAGC A CUGAGGGU | 5199 | ACCCUCAG CUGAUGAGGCCGUUAGGCCGAA ICUACAAA | 14539 |
| 5056 | UGUAGCAC U GAGGGUGG | 5200 | CCACCCUC CUGAUGAGGCCGUUAGGCCGAA IUGCUACA | 14540 |
| 5066 | AGGGUGGC A CUCAACUC | 5201 | GAGUUGAG CUGAUGAGGCCGUUAGGCCGAA ICCACCCU | 14541 |
| 5068 | GGUGGCAC U CAACUCUG | 5202 | CAGAGUUG CUGAUGAGGCCGUUAGGCCGAA IUGCCACC | 14542 |
| 5070 | UGGCACUC A ACUCUGAG | 5203 | CUCAGAGU CUGAUGAGGCCGUUAGGCCGAA IAGUGCCA | 14543 |
| 5073 | CACUCAAC U CUGAGCCC | 5204 | GGGCUCAG CUGAUGAGGCCGUUAGGCCGAA IUUGAGUG | 14544 |
| 5075 | CUCAACUC U GAGCCCAU | 5205 | AUGGGCUC CUGAUGAGGCCGUUAGGCCGAA IAGUUGAG | 14545 |
| 5080 | CUCUGAGC C CAUACUUU | 5206 | AAAGUAUG CUGAUGAGGCCGUUAGGCCGAA ICUCAGAG | 14546 |
| 5081 | UCUGAGCC C AUACUUUU | 5207 | AAAAGUAU CUGAUGAGGCCGUUAGGCCGAA IGCUCAGA | 14547 |
| 5082 | CUGAGCCC A UACUUUUG | 5208 | CAAAAGUA CUGAUGAGGCCGUUAGGCCGAA IGGCUCAG | 14548 |
| 5086 | GCCCAUAC U UUUGGCUC | 5209 | GAGCCAAA CUGAUGAGGCCGUUAGGCCGAA IUAUGGGC | 14549 |
| 5093 | CUUUUGGC U CCUCUAGU | 5210 | ACUAGAGG CUGAUGAGGCCGUUAGGCCGAA ICCAAAAG | 14550 |
| 5095 | UUUGGCUC C UCUAGUAA | 5211 | UUACUAGA CUGAUGAGGCCGUUAGGCCGAA IAGCCAAA | 14551 |
| 5096 | UUGGCUCC U CUAGUAAG | 5212 | CUUACUAG CUGAUGAGGCCGUUAGGCCGAA IGAGCCAA | 14552 |
| 5098 | GGCUCCUC U AGUAAGAU | 5213 | AUCUUACU CUGAUGAGGCCGUUAGGCCGAA IAGGAGCC | 14553 |
| 5109 | UAAGAUGC A CUGAAAAC | 5214 | GUUUUCAG CUGAUGAGGCCGUUAGGCCGAA ICAUCUUA | 14554 |
| 5111 | AGAUGCAC U GAAAACUU | 5215 | AAGUUUUC CUGAUGAGGCCGUUAGGCCGAA IUGCAUCU | 14555 |
| 5118 | CUGAAAAC U UAGCCAGA | 5216 | UCUGGCUA CUGAUGAGGCCGUUAGGCCGAA IUUUUCAG | 14556 |
| 5123 | AACUUAGC C AGAGUUAG | 5217 | CUAACUCU CUGAUGAGGCCGUUAGGCCGAA ICUAAGUU | 14557 |
| 5124 | ACUUAGCC A GAGUUAGG | 5218 | CCUAACUC CUGAUGAGGCCGUUAGGCCGAA IGCUAAGU | 14558 |
| 5138 | AGGUUGUC U CCAGGCCA | 5219 | UGGCCUGG CUGAUGAGGCCGUUAGGCCGAA IACAACCU | 14559 |
| 5140 | GUUGUCUC C AGGCCAUG | 5220 | CAUGGCCU CUGAUGAGGCCGUUAGGCCGAA IAGACAAC | 14560 |
| 5141 | UUGUCUCC A GGCCAUGA | 5221 | UCAUGGCC CUGAUGAGGCCGUUAGGCCGAA IGAGACAA | 14561 |
| 5145 | CUCCAGGC C AUGAUGGC | 5222 | GCCAUCAU CUGAUGAGGCCGUUAGGCCGAA ICCUGGAG | 14562 |
| 5146 | UCCAGGCC A UGAUGGCC | 5223 | GGCCAUCA CUGAUGAGGCCGUUAGGCCGAA IGCCUGGA | 14563 |
| 5154 | AUGAUGGC C CUACACUG | 5224 | CAGUGUAA CUGAUGAGGCCGUUAGGCCGAA ICCAUCAU | 14564 |
| 5155 | UGAUGGCC U UACACUGA | 5225 | UCAGUGUA CUGAUGAGGCCGUUAGGCCGAA IGCCAUCA | 14565 |
| 5159 | GGCCUUAC A CUGAAAAU | 5226 | AUUUUCAG CUGAUGAGGCCGUUAGGCCGAA IUAAGGCC | 14566 |
| 5161 | CCUUACAC U GAAAAUGU | 5227 | ACAUUUUC CUGAUGAGGCCGUUAGGCCGAA IUGUAAGG | 14567 |
| 5171 | AAAAUGUC A CAUUCUAU | 5228 | AUAGAAUG CUGAUGAGGCCGUUAGGCCGAA IACAUUUU | 14568 |
| 5173 | AAUGUCAC A UUCUAUUU | 5229 | AAAUAGAA CUGAUGAGGCCGUUAGGCCGAA IUGACAUU | 14569 |
| 5177 | UCACAUUC U AUUUUGGG | 5230 | CCCAAAAU CUGAUGAGGCCGUUAGGCCGAA IAAUGUGA | 14570 |
| 5201 | AUAUAGUC C AGACACUU | 5231 | AAGUGUCU CUGAUGAGGCCGUUAGGCCGAA IACUAUAU | 14571 |
| 5202 | UAUAGUCC A GACACUUA | 5232 | UAAGUGUC CUGAUGAGGCCGUUAGGCCGAA IGACUAUA | 14572 |
| 5206 | GUCCAGAC A CUUAACUC | 5233 | GAGUUAAG CUGAUGAGGCCGUUAGGCCGAA IUCUGGAC | 14573 |
| 5208 | CCAGACAC U UAACUCAA | 5234 | UUGAGUUA CUGAUGAGGCCGUUAGGCCGAA IUGUCUGG | 14574 |
| 5213 | CACUUAAC U CAAUUUCU | 5235 | AGAAAUUG CUGAUGAGGCCGUUAGGCCGAA IUUAAGUG | 14575 |
| 5215 | CUUAACUC A AUUUCUUG | 5236 | CAAGAAAU CUGAUGAGGCCGUUAGGCCGAA IAGUUAAG | 14576 |
| 5221 | UCAAUUUC U UGGUAUUA | 5237 | UAAUACCA CUGAUGAGGCCGUUAGGCCGAA IAAAUUGA | 14577 |
| 5233 | UAUUAUUC U GUUUUGCA | 5238 | UGCAAAAC CUGAUGAGGCCGUUAGGCCGAA IAAUAAUA | 14578 |
| 5241 | UGUUUUGC A CAGUUAGU | 5239 | ACUAACUG CUGAUGAGGCCGUUAGGCCGAA ICAAAACA | 14579 |
| 5243 | UUUUGCAC A GUUAGUUG | 5240 | CAACUAAC CUGAUGAGGCCGUUAGGCCGAA IUGCAAAA | 14580 |
| 5263 | AAGAAAGC U GAGAAGAA | 5241 | UUCUUCUC CUGAUGAGGCCGUUAGGCCGAA ICUUUCUU | 14581 |
| 5281 | GAAAAUGC A GUCCUGAG | 5242 | CUCAGGAC CUGAUGAGGCCGUUAGGCCGAA ICAUUUUC | 14582 |
| 5285 | AUGCAGUC C UGAGGAGA | 5243 | UCUCCUCA CUGAUGAGGCCGUUAGGCCGAA IACUGCAU | 14583 |
| 5286 | UGCAGUCC U GAGGAGAG | 5244 | CUCUCCUC CUGAUGAGGCCGUUAGGCCGAA IGACUGCA | 14584 |
| 5300 | GAGUUUUC U CCAUAUCA | 5245 | UGAUAUGG CUGAUGAGGCCGUUAGGCCGAA IAAAACUC | 14585 |
| 5302 | GUUUUCUC C AUAUCAAA | 5246 | UUUGAUAU CUGAUGAGGCCGUUAGGCCGAA IAGAAAAC | 14586 |
| 5303 | UUUUCUCC A UAUCAAAA | 5247 | UUUUGAUA CUGAUGAGGCCGUUAGGCCGAA IGAGAAAA | 14587 |
| 5308 | UCCAUAUC A AAACGAGG | 5248 | CCUCGUUU CUGAUGAGGCCGUUAGGCCGAA IAUAUGGA | 14588 |
| 5319 | ACGAGGGC U GAUGGAGG | 5249 | CCUCCAUC CUGAUGAGGCCGUUAGGCCGAA ICCCUCGU | 14589 |
| 5337 | AAAAGGUC A AUAAGGUC | 5250 | GACCUUAU CUGAUGAGGCCGUUAGGCCGAA IACCUUUU | 14590 |
| 5346 | AUAAGGUC A AGGGAAGA | 5251 | UCUUCCCU CUGAUGAGGCCGUUAGGCCGAA IACCUUAU | 14591 |
| 5356 | GGGAAGAC C CCGUCUCU | 5252 | AGAGACGG CUGAUGAGGCCGUUAGGCCGAA IUCUUCCC | 14592 |
| 5357 | GGAAGACC C CGUCUCUA | 5253 | UAGAGACG CUGAUGAGGCCGUUAGGCCGAA IGUCUUCC | 14593 |
| 5358 | GAAGACCC C GUCUCUAU | 5254 | AUAGAGAC CUGAUGAGGCCGUUAGGCCGAA IGGUCUUC | 14594 |
| 5362 | ACCCCGUC U CUAUACCA | 5255 | UGGUAUAG CUGAUGAGGCCGUUAGGCCGAA IACGGGGU | 14595 |
| 5364 | CCCGUCUC U AUACCAAC | 5256 | GUUGGUAU CUGAUGAGGCCGUUAGGCCGAA IAGACGGG | 14596 |
| 5369 | CUCUAUAC C AACCAAAC | 5257 | GUUUGGUU CUGAUGAGGCCGUUAGGCCGAA IUAUAGAG | 14597 |
| 5370 | UCUAUACC A ACCAAACC | 5258 | GGUUUGGU CUGAUGAGGCCGUUAGGCCGAA IGUAUAGA | 14598 |
| 5373 | AUACCAAC C AAACCAAU | 5259 | AUUGGUUU CUGAUGAGGCCGUUAGGCCGAA IUUGGUAU | 14599 |
| 5374 | UACCAACC A AACCAAUU | 5260 | AAUUGGUU CUGAUGAGGCCGUUAGGCCGAA IGUUGGUA | 14600 |
| 5378 | AACCAAAC C AAUUCACC | 5261 | GGUGAAUU CUGAUGAGGCCGUUAGGCCGAA IUUUGGUU | 14601 |
| 5379 | ACCAAACC A AUUCACCA | 5262 | UGGUGAAU CUGAUGAGGCCGUUAGGCCGAA IGUUUGGU | 14602 |
| 5384 | ACCAAUUC A CCAACACA | 5263 | UGUGUUGG CUGAUGAGGCCGUUAGGCCGAA IAAUUGGU | 14603 |
| 5386 | CAAUUCAC C AACACAGU | 5264 | ACUGUGUU CUGAUGAGGCCGUUAGGCCGAA IUGAAUUG | 14604 |
| 5387 | AAUUCACC A ACACAGUU | 5265 | AACUGUGU CUGAUGAGGCCGUUAGGCCGAA IGUGAAUU | 14605 |
| 5390 | UCACCAAC A CAGUUGGG | 5266 | CCCAACUG CUGAUGAGGCCGUUAGGCCGAA IUUGGUGA | 14606 |
| 5392 | ACCAACAC A GUUGGGAC | 5267 | GUCCCAAC CUGAUGAGGCCGUUAGGCCGAA IUGUUGGU | 14607 |
| 5401 | GUUGGGAC C CAAAACAC | 5268 | GUGUUUUG CUGAUGAGGCCGUUAGGCCGAA IUCCCAAC | 14608 |
| 5402 | UUGGGACC C AAAACACA | 5269 | UGUGUUUU CUGAUGAGGCCGUUAGGCCGAA IGUCCCAA | 14609 |
| 5403 | UGGGACCC A AAACACAG | 5270 | CUGUGUUU CUGAUGAGGCCGUUAGGCCGAA IGGUCCCA | 14610 |
| 5408 | CCCAAAAC A CAGGAAGU | 5271 | ACUUCCUG CUGAUGAGGCCGUUAGGCCGAA IUUUUGGG | 14611 |
| 5410 | CAAAACAC A GGAAGUCA | 5272 | UGACUUCC CUGAUGAGGCCGUUAGGCCGAA IUGUUUUG | 14612 |
| 5418 | AGGAAGUC A GUCACGUU | 5273 | AACGUGAC CUGAUGAGGCCGUUAGGCCGAA IACUUCCU | 14613 |
| 5422 | AGUCAGUC A CGUUUCCU | 5274 | AGGAAACG CUGAUGAGGCCGUUAGGCCGAA IACUGACU | 14614 |
| 5429 | CACGUUUC C UUUUCAUU | 5275 | AAUGAAAA CUGAUGAGGCCGUUAGGCCGAA IAAACGUG | 14615 |
| 5430 | ACGUUUCC U UUUCAUUU | 5276 | AAAUGAAA CUGAUGAGGCCGUUAGGCCGAA IGAAACGU | 14616 |
| 5435 | UCCUUUUC A UUUAAUGG | 5277 | CCAUUAAA CUGAUGAGGCCGUUAGGCCGAA IAAAAGGA | 14617 |
| 5450 | GGGGAUUC C ACUAUCUC | 5278 | GAGAUAGU CUGAUGAGGCCGUUAGGCCGAA IAAUCCCC | 14618 |
| 5451 | GGGAUUCC A CUAUCUCA | 5279 | UCAGAUAG CUGAUGAGGCCGUUAGGCCGAA IGAAUCCC | 14619 |
| 5453 | GAUUCCAC U AUCUCACA | 5280 | UGUGAGAU CUGAUGAGGCCGUUAGGCCGAA IUGGAAUC | 14620 |
| 5457 | CCACUAUC U CACACUAA | 5281 | UUAGUGUG CUGAUGAGGCCGUUAGGCCGAA IAUAGUGG | 14621 |
| 5459 | ACUAUCUC A CACUAAUC | 5282 | GAUUAGUG CUGAUGAGGCCGUUAGGCCGAA IAGAUAGU | 14622 |
| 5461 | UAUCUCAC A CUAAUCUG | 5283 | CAGAUUAG CUGAUGAGGCCGUUAGGCCGAA IUGAGAUA | 14623 |
| 5463 | UCUCACAC U AAUCUGAA | 5284 | UUCAGAUU CUGAUGAGGCCGUUAGGCCGAA IUGUGAGA | 14624 |
| 5468 | CACUAAUC U GAAAGGAU | 5285 | AUCCUUUC CUGAUGAGGCCGUUAGGCCGAA IAUUAGUG | 14625 |
| 5487 | GGAAGAGC A UUAGCUGG | 5286 | CCAGCUAA CUGAUGAGGCCGUUAGGCCGAA ICUCUUCC | 14626 |
| 5493 | GCAUUAGC U GGCGCAUA | 5287 | UAUGCGCC CUGAUGAGGCCGUUAGGCCGAA ICUAAUGC | 14627 |
| 5499 | GCUGGCGC A UAUUAAGC | 5288 | GCUUAAUA CUGAUGAGGCCGUUAGGCCGAA ICGCCAGC | 14628 |
| 5508 | UAUUAAGC A CUUUAAGC | 5289 | GCUUAAAG CUGAUGAGGCCGUUAGGCCGAA ICUUAAUA | 14629 |
| 5510 | UUAAGCAC U UUAAGCUC | 5290 | GAGCUUAA CUGAUGAGGCCGUUAGGCCGAA IUGCUUAA | 14630 |
| 5517 | CUUUAAGC U CCUUGAGU | 5291 | ACUCAAGG CUGAUGAGGCCCUUAGGCCGAA ICUUAAAG | 14631 |
| 5519 | UUAAGCUC C UUGAGUAA | 5292 | UUACUCAA CUGAUGAGGCCGUUAGGCCGAA IAGCUUAA | 14632 |
| 5520 | UAAGCUCC U UGAGUAAA | 5293 | UUUACUCA CUGAUGAGGCCGUUAGGCCGAA IGAGCUUA | 14633 |
| 5550 | AUUUAUGC A AGGUAUUU | 5294 | AAAUACCU CUGAUGAGGCCGUUAGGCCGAA ICAUAAAU | 14634 |
| 5560 | GGUAUUUC U CCAGUUGG | 5295 | CCAACUGG CUGAUGAGGCCGUUAGGCCGAA IAAAUACC | 14635 |
| 5562 | UAUUUCUC C AGUUGGGA | 5296 | UCCCAACU CUGAUGAGGCCGUUAGGCCGAA IAGAAAUA | 14636 |
| 5563 | AUUUCUCC A GUUGGGAC | 5297 | GUCCCAAC CUGAUGAGGCCGUUAGGCCGAA IGAGAAAU | 14637 |
| 5572 | GUUGGGAC U CAGGAUAU | 5298 | AUAUCCUG CUGAUGAGGCCGUUAGGCCGAA IUCCCAAC | 14638 |
| 5574 | UGGGACUC A GGAUAUUA | 5299 | UAAUAUCC CUGAUGAGGCCGUUAGGCCGAA IAGUCCCA | 14639 |
| 5593 | UPAUGAGC C AUCACUAG | 5300 | CUAGUGAU CUGAUGAGGCCGUUAGGCCGAA ICUCAUUA | 14640 |
| 5594 | AAUGAGCC A UCACUAGA | 5301 | UCUAGUGA CUGAUGAGGCCGUUAGGCCGAA IGCUCAUU | 14641 |
| 5597 | GAGCCAUC A CUAGAAGA | 5302 | UCUUCUAG CUGAUGAGGCCGUUAGGCCGAA IAUGGCUC | 14642 |
| 5599 | GCCAUCAC U AGAAGAAA | 5303 | UUUCUUCU CUGAUGAGGCCGUUAGGCCGAA IUGAUGGC | 14643 |
| 5611 | AGAAAAGC C CAUUUUCA | 5304 | UGAAAAUG CUGAUGAGGCCGUUAGGCCGAA ICUUUUCU | 14644 |
| 5612 | GAAAAGCC C AUUUUCAA | 5305 | UUGAAAAU CUGAUGAGGCCGUUAGGCCGAA IGCUUUUC | 14645 |
| 5613 | AAAAGCCC A UUUUCAAC | 5306 | GUUGAAAA CUGAUGAGGCCGUUAGGCCGAA IGGCUUUU | 14646 |
| 5619 | CCAUUUUC A ACUGCUUU | 5307 | AAAGCAGU CUGAUGAGGCCGUUAGGCCGAA IAAAAUGG | 14647 |
| 5622 | UUUUCAAC U GCUUUGAA | 5308 | UUCAAAGC CUGAUGAGGCCGUUAGGCCGAA IUUGAAAA | 14648 |
| 5625 | UCAACUGC U UUGAAACU | 5309 | AGUUUCAA CUGAUGAGGCCGUUAGGCCGAA ICAGUUGA | 14649 |
| 5633 | UUUGAAAC U UGCCUGGG | 5310 | CCCAGGCA CUGAUGAGGCCGUUAGGCCGAA IUUUCAAA | 14650 |
| 5637 | AAACUUGC C UGGGGUCU | 5311 | AGACCCCA CUGAUGAGGCCGUUAGGCCGAA ICAAGUUU | 14651 |
| 5638 | AACUUGCC U GUGGUCUG | 5312 | CAGACCCC CUGAUGAGGCCGUUAGGCCGAA IGCAAGUU | 14652 |
| 5645 | CUGGGGUC U GAGCAUGA | 5313 | UCAUGCUC CUGAUGAGGCCGUUAGGCCGAA IACCCCAG | 14653 |
| 5650 | GUCUGAGC A UGAUGGGA | 5314 | UCCCAUCA CUGAUGAGGCCGUUAGGCCGAA ICUCAGAC | 14654 |
| 5669 | AGGGAGAC A GGGUAGGA | 5315 | UCCUACCC CUGAUGAGGCCGUUAGGCCGAA IUCUCCCU | 14655 |
| 5686 | AAGGGCGC C UACUCUUC | 5316 | GAAGAGUA CUGAUGAGGCCGUUAGGCCGAA ICGCCCUU | 14656 |
| 5687 | AGGGCGCC U ACUCUUCA | 5317 | UGAAGAGU CUGAUGAGGCCGUUAGGCCGAA IGCGCCCU | 14657 |
| 5690 | GCGCCUAC U CUUCAGGG | 5318 | CCCUGAAG CUGAUGAGGCCGUUAGGCCGAA IUAGGCGC | 14658 |
| 5692 | GCCUACUC U UCAGGGUC | 5319 | GACCCUGA CUGAUGAGGCCGUUAGGCCGAA IAGUAGGC | 14659 |
| 5695 | UACUCUUC A GGGUCUAA | 5320 | UUAGACCC CUGAUGAGGCCGUUAGGCCGAA IAAGAGUA | 14660 |
| 5701 | UCAGGGUC U AAAGAUCA | 5321 | UGAUCUUU CUGAUGAGGCCGUUAGGCCGAA IACCCUGA | 14661 |
| 5709 | UAAAGAUC A AGUGGGCC | 5322 | GGCCCACU CUGAUGAGGCCGUUAGGCCGAA IAUCUUUA | 14662 |
| 5717 | AAGUGGGC C UUGGAUCG | 5323 | CGAUCCAA CUGAUGAGGCCGUUAGGCCGAA ICCCACUU | 14663 |
| 5718 | AGUGGGCC U UGGAUCGC | 5324 | GCGAUCCA CUGAUGAGGCCGUUAGGCCGAA IGCCCACU | 14664 |
| 5727 | UGGAUCGC U AAGCUGGC | 5325 | GCCAGCUU CUGAUGAGGCCGUUAGGCCGAA ICGAUCCA | 14665 |
| 5732 | CGCUAAGC U GCCUCUGU | 5326 | ACAGAGCC CUGAUGAGGCCGUUAGGCCGAA ICUUAGCG | 14666 |
| 5736 | AAGCUGGC U CUGUUUGA | 5327 | UCAAACAG CUGAUGAGGCCGUUAGGCCGAA ICCAGCUU | 14667 |
| 5738 | GCUGGCUC U GUUUGAUG | 5328 | CAUCAAAC CUGAUGAGGCCGUUAGGCCGAA IAGCCAGC | 14668 |
| 5748 | UUUGAUGC U AUUUAUGC | 5329 | GCAUAAAU CUGAUGAGGCCGUUAGGCCGAA ICAUCAAA | 14669 |
| 5757 | AUUUAUGC A AGUUAGGU | 5330 | CCCUAACU CUGAUGAGGCCGUUAGGCCGAA ICAUAPAU | 14670 |
| 5768 | UUAGCGUC U AUGUAUUU | 5331 | AAAUACAU CUGAUGAGGCCGUUAGGCCGAA IACCCUAA | 14671 |
| 5786 | GGAUGCGC C UACUCUUC | 5332 | GAAGAGUA CUGAUGAGGCCGUUAGGCCGAA ICGCAUCC | 14672 |
| 5787 | GAUGCGCC U ACUCUUCA | 5333 | UGAACACU CUGAUGAGGCCGUUAGGCCGAA IGCGCAUC | 14673 |
| 5790 | GCGCCUAC U CUUCAGGG | 5318 | CCCUAAGG CUGAUGAGGCCGUUAGGCCGAA IUAGCCGC | 14658 |
| 5792 | GCCUACUC U UCAGGGUC | 5319 | GACCCUGA CUGAUGAGGCCGUUAGGCCGAA IAGUAGGC | 14659 |
| 5795 | UACUCUUC A GGGUCUAA | 5320 | UUAGACCC CUGAUGAGGCCGUUAGGCCGAA IAAGAGUA | 14660 |
| 5801 | UCAGGGUC U AAAGAUCA | 5321 | UGAUCUUU CUGAUGAGGCCGUUAGGCCGAA IACCCUGA | 14661 |
| 5809 | UAAAGAUC A AGUCGGCC | 5322 | GCCCCACU CUGAUGAGGCCGUUAGGCCGAA IAUCUUUA | 14662 |
| 5817 | AAGUGGGC C UUGGAUCG | 5323 | CGAUCCAA CUGAUGAGGCCGUUAGGCCGAA ICCCACUU | 14663 |
| 5818 | AGUGGGCC U UGGAUCGC | 5324 | GCGAUCCA CUGAUGAGGCCGUUAGGCCGAA IGCCCACU | 14664 |
| 5827 | UGGAUCGC U AAGCUGGC | 5325 | GCCAGCUU CUGAUGAGGCCGUUAGGCCGAA ICCAUCCA | 14665 |
| 5832 | CGCUAAGC U GGCUCUGU | 5326 | ACACAGCC CUGAUGAGGCCGUUAGGCCGAA ICUUAGCG | 14666 |
| 5836 | AAGCUGGC U CUGUUUGA | 5327 | UCAAACAG CUGAUGAGGCCGUUAGGCCGAA ICCAGCUU | 14667 |
| 5838 | GCUGGCUC U GUUUGAUG | 5328 | CAUCAAAC CUGAUGAGGCCGUUAGGCCGAA IAGCCAGC | 14668 |
| 5848 | UUUGAUCC U AUUUAUGC | 5329 | GCAUAAAU CUGAUGAGGCCGUUAGGCCGAA ICAUCAAA | 14669 |
| 5857 | AUUUAUGC A AGUUACGG | 5330 | CCCUAACU CUGAUGAGGCCGUUAGGCCGAA ICAUAAAU | 14670 |
| 5868 | UUAGGGUC U AUGUAUUU | 5331 | AAAUACAU CUGAUGACGCCGUUAGGCCGAA IACCCUAA | 14671 |
| 5885 | AGGAUGUC U GCACCUUC | 5334 | GAAGGUGC CUGAUGAGGCCGUUAGGCCGAA IACAUCCU | 14674 |
| 5888 | AUGUCUGG A CCUUCUGC | 5335 | GCAGAAGG CUGAUGAGGCCGUUAGGCCGAA ICAGACAU | 14675 |
| 5890 | GUCUGCAC C UUCUGCAG | 5336 | CUGCAGAA CUGAUGAGGCCGUUAGGCCGAA IUGCAGAC | 14676 |
| 5891 | UCUGCACC U UCUGCAGC | 5337 | GCUGCAGA CUGAUGAGGCCGUUAGGCCGAA IGUCCAGA | 14677 |
| 5894 | GCACCUUC U GCAGCCAG | 5338 | CUGGCUGC CUGAUGAGGCCGUUAGGCCGAA IAAGGUGC | 14678 |
| 5897 | CCUUCUGC A GCCAGUCA | 5339 | UGACUGGC CUGAUGAGGCCGUUAGGCCGAA ICAGAAGG | 14679 |
| 5900 | UCUGCAGC C AGUCAGAA | 5340 | UUCUGACU CUGAUGAGGCCGUUAGGCCGAA ICUGCAGA | 14680 |
| 5901 | CUGCAGCC A GUCAGAAG | 5341 | CUUCUGAC CUGAUGAGGCCGUUAGGCCGAA IGCUGCAG | 14681 |
| 5905 | AGCCAGUC A GAAGCUGG | 5342 | CCAGCUUC CUGAUGAGGCCGUUAGGCCGAA IACUGGCU | 14682 |
| 5911 | UCAGAAGC U GGAGAGGC | 5343 | GCCUCUCC CUGAUGAGGCCGUUAGGCCGAA ICUUCUGA | 14683 |
| 5920 | GGAGAGGC A ACAGUGGA | 5344 | UCCACUGU CUGAUGAGGCCGUUAGGCCGAA ICCUCUCC | 14684 |
| 5923 | GAGGCAAC A GUGGAUUG | 5345 | CAAUCCAC CUGAUGAGGCCGUUAGGCCGAA IUUGCCUC | 14685 |
| 5933 | UGGAUUGC U GCUUCUUG | 5346 | CAAGAAGC CUGAUGAGGCCGUUAGGCCGAA ICAAUCCA | 14686 |
| 5936 | AUUGCUGC U UCUUGGGG | 5347 | CCCCAAGA CUGAUGAGGCCGUUAGGCCGAA ICAGCAAU | 14687 |
| 5939 | GCUGCUUC U UGGGGAGA | 5348 | UCUCCCCA CUGAUGAGGCCGUUAGGCCGAA IAAGCAGC | 14688 |
| 5957 | GAGUAUGC U UCCUUUUA | 5349 | UAAAAGGA CUGAUGAGGCCGUUAGGCCGAA ICAUACUC | 14689 |
| 5960 | UAUGCUUC C UUUUAUCC | 5350 | GGAUAAAA CUGAUGAGGCCGUUAGGCCGAA IAAGCAUA | 14690 |
| 5961 | AUGCUUCC U UUUAUCCA | 5351 | UGGAUAAA CUGAUGAGGCCGUUAGGCCGAA IGAAGCAU | 14691 |
| 5968 | CUUUUAUC C AUGUAAUU | 5352 | AAUUACAU CUGAUGAGGCCGUUAGGCCGAA IAUAAAAG | 14692 |
| 5969 | UUUUAUCC A UGUAAUUU | 5353 | AAAUUACA CUGAUGAGGCCGUUAGGCCGAA IGAUAAAA | 14693 |
| 5981 | AAUUUAAC U GUAGAACC | 5354 | GGUUCUAC CUGAUGAGGCCGUUAGGCCGAA IUUAAAUU | 14694 |
| 5989 | UGUAGAAC C UGAGCUCU | 5355 | AGAGCUCA CUGAUGAGGCCGUUAGGCCGAA IUUCUACA | 14695 |
| 5990 | GUAGAACC U GAGCUCUA | 5356 | UAGAGCUC CUGAUGAGGCCGUUAGGCCGAA IGUUCUAC | 14696 |
| 5995 | ACCUGAGC U CUAAGUAA | 5357 | UUACUUAG CUGAUGAGGCCGUUAGGCCGAA ICUCAGGU | 14697 |
| 5997 | CUGAGCUC U AAGUAACC | 5358 | GGUUACUU CUGAUGAGGCCGUUAGGCCGAA IAGCUCAG | 14698 |
| 6005 | UAAGUAAC C GAAGAAUG | 5359 | CAUUCUUC CUGAUGAGGCCGUUAGGCCGAA IUUACUUA | 14699 |
| 6019 | AUGUAUGC C UCUGUUCU | 5360 | AGAACAGA CUGAUGAGGCCGUUAGGCCGAA ICAUACAU | 14700 |
| 6020 | UGUAUGCC U CUGUUCUU | 5361 | AAGAACAG CUGAUGAGGCCGUUAGGCCGAA IGCAUACA | 14701 |
| 6022 | UAUGCCUC U GUUCUUAU | 5362 | AUAAGAAC CUGAUGAGGCCGUUAGGCCGAA IAGCCAUA | 14702 |
| 6027 | CUCUGUUC U UAUGUGCC | 5363 | GGCACAUA CUGAUGAGGCCGUUAGGCCGAA IAACAGAG | 14703 |
| 6035 | UUAUGUGC C ACAUCCUU | 5364 | AAGGAUGU CUGAUGAGGCCGUUAGGCCGAA ICACAUAA | 14704 |
| 6036 | UAUGUGCC A CAUCCUUG | 5365 | CAAGCAUG CUGAUGAGGCCGUUAGGCCGAA IGCACAUA | 14705 |
| 6038 | UGUGCCAC A UCCUUGUU | 5366 | AACAAGGA CUGAUGAGGCCGUUAGGCCGAA IUGGCACA | 14706 |
| 6041 | GCCACAUC C UUGUUUAA | 5367 | UUAAACAA CUGAUGAGGCCGUUAGGCCGAA IAUGUUGC | 14707 |
| 6042 | CCACAUCC U UGUUUAAA | 5368 | UUUAAACA CUGAUGAGGCCGUUAGGCCGAA IGAUGUGG | 14708 |
| 6054 | UUAAAGGC U CUCUGUAU | 5369 | AUACAGAG CUGAUGAGGCCGUUAGGCCGAA ICCUUUAA | 14709 |
| 6056 | AAAGGCUC U CUGUAUGA | 5370 | UCAUACAG CUGAUGAGGCCGUUAGGCCGAA IAGCCUUU | 14710 |
| 6058 | ACGCUCUC U GUAUGAAG | 5371 | CUUCAUAC CUGAUGAGGCCGUUAGGCCGAA IAGAGCCU | 14711 |
| 6076 | GAUGUGAC C GUCAUCAG | 5372 | CUGAUGAC CUGAUGAGGCCGUUAGGCCGAA IUCCCAUC | 14712 |
| 6080 | GGACCGUC A UCAGCACA | 5373 | UGUGCUGA CUGAUGAGGCCGUUAGGCCGAA IACGGUCC | 14713 |
| 6083 | CCGUCAUC A GCACAUUC | 5374 | GAAUGUGC CUGAUGAGGCCGUUAGGCCGAA IAUGACGG | 14714 |
| 6086 | UCAUCAGC A CAUUCCCU | 5375 | AGGGAAUG CUGAUGAGGCCGUUAGGCCGAA ICUGAUGA | 14715 |
| 6088 | AUCAGCAC A UUCCCUAG | 5376 | CUAGGGAA CUGAUGAGGCCGUUAGGCCGAA IUGCUGAU | 14716 |
| 6092 | UCACAGUC C CUAGUGAG | 5377 | CUCACUAG CUGAUGAGGCCGUUAGGCCGAA IAAUGUGC | 14717 |
| 6093 | CACAUUCC C UAGUGAGC | 5378 | GCUCACUA CUGAUGAGGCCGUUAGGCCGAA IGAAUGUG | 14718 |
| 6094 | ACAUUCCC U AGUGAGCC | 5379 | GGCUCACU CUGAUGAGGCCGUUAGGCCGAA IGGAAUGU | 14719 |
| 6102 | UAGUGAGC C UACUGGCU | 5380 | AGCCAGUA CUGAUGAGGCCGUUAGGCCGAA ICUCACUA | 14720 |
| 6103 | AGUGAGCC U ACUGGCUC | 5381 | GAGCCAGU CUGAUGAGGCCGUUAGGCCGAA IGCUCACU | 14721 |
| 6106 | GAGCCUAC U GGCUCCUG | 5382 | CAGGAGCC CUGAUGAGGCCGUUAGGCCGAA IUAGGCUC | 14722 |
| 6110 | CUACUGGC U CCUGGCAG | 5383 | CUGCCAGG CUGAUGAGGCCGUUAGGCCGAA ICCAGUAG | 14723 |
| 6112 | ACUGGCUC C UGGCAGCG | 5384 | CGCUGCCA CUGAUGAGGCCGUUAGGCCGAA IAGCCAGU | 14724 |
| 6113 | CUGGCUCC U GGCAGCGG | 5385 | CCGCUGCC CUGAUGAGGCCGUUAGGCCGAA IGAGCCAG | 14725 |
| 6117 | CUCCUGGC A GCGGCUUU | 5386 | AAAGCCGC CUGAUGAGGCCGUUAGGCCGAA ICCAGGAG | 14726 |
| 6123 | GCAGCGGC U UGUGUGGA | 5387 | UCCACAAA CUGAUGAGGCCGUUAGGCCGAA ICCGCUGC | 14727 |
| 6136 | UGGAAGAC U CACUAGCC | 5388 | GGCUAGUG CUGAUGAGGCCGUUAGGCCGAA IUCUUCCA | 14728 |
| 6138 | GAAGACUC A CUAGCCAG | 5389 | CUGGCUAG CUGAUGAGGCCGUUAGGCCGAA IAGUCUUC | 14729 |
| 6140 | AGACUCAC U AGCCAGAA | 5390 | UUCUGGCU CUGAUGAGGCCGUUAGGCCGAA IUGAGUCU | 14730 |
| 6144 | UCACUAGC C AGAAGAGA | 5391 | UCUCUUCU CUGAUGAGGCCGUUAGGCCGAA ICUAGUGA | 14731 |
| 6145 | CACUAGCC A GAAGAGAG | 5392 | CUCUCUUC CUGAUGAGGCCGUUAGGCCGAA IGCUAGUG | 14732 |
| 6163 | AGUGGGAC A GUCCUCUC | 5393 | GAGAGGAC CUGAUGAGGCCGUUAGGCCGAA IUCCCACU | 14733 |
| 6167 | GGACAGUC C UCUCCACC | 5394 | GGUGGAGA CUGAUGAGGCCGUUAGGCCGAA IACUGUCC | 14734 |
| 6168 | GACAGUCC U CUCCACCA | 5395 | UGGUGGAG CUGAUGAGGCCGUUAGGCCGAA IGACUGUC | 14735 |
| 6170 | CAGUCCUC U CCACCAAG | 5396 | CUUGGUGG CUGAUGAGGCCGUUAGGCCGAA IAGGACUG | 14736 |
| 6172 | GUCCUCUC C ACCAAGAU | 5397 | AUCUUGGU CUGAUGAGGCCGUUAGGCCGAA IAGAGGAC | 14737 |
| 6173 | UCCUCUCC A CCAAGAUC | 5398 | GAUCUUGG CUGAUGAGGCCGUUAGGCCGAA IGAGAGGA | 14738 |
| 6175 | CUCUCCAC C AAGAUCUA | 5399 | UAGAUCUU CUGAUGAGGCCGUUAGGCCGAA IUGGAGAG | 14739 |
| 6176 | UCUCCACC A AGAUCUAA | 5400 | UUAGAUCU CUGAUGAGGCCGUUAGGCCGAA IGUGGAGA | 14740 |
| 6182 | CCAAGAUC U AAAUCCAA | 5401 | UUGGAUUU CUGAUGAGGCCGUUAGGCCGAA IAUCUUGG | 14741 |
| 6188 | UCUAAAUC C AAACAAAA | 5402 | UUUUGUUU CUGAUGAGGCCGUUAGGCCGAA IAUUUAGA | 14742 |
| 6189 | CUAAAUCC A AACAAAAG | 5403 | CUUUUGUU CUGAUGAGGCCGUUAGGCCGAA IGAUUUAG | 14743 |
| 6193 | AUCCAAAC A AAAGCAGG | 5404 | CCUGCUUU CUGAUGAGGCCGUUAGGCCGAA IUUUGGAU | 14744 |
| 6199 | ACAAAAGC A GGCUAGAG | 5405 | CUCUAGCC CUGAUGAGGCCGUUAGGCCGAA ICU1IUUGU | 14745 |
| 6203 | AAGCAGGC U AGAGCCAG | 5406 | CUGGCUCU CUGAUGAGGCCGUUAGGCCGAA ICCUGCUU | 14746 |
| 6209 | GCUAGAGC C AGAAGAGA | 5407 | UCUCUUCU CUGAUGAGGCCGUUAGGCCGAA ICUCUAGC | 14747 |
| 6210 | CUAGAGCC A GAAGAGAG | 5408 | CUCUCUUC CUGAUGAGGCCGUUAGGCCGAA IGCUCUAG | 14748 |
| 6222 | GAGAGGAC A AAUCUUUG | 5409 | CAAAGAUU CUGAUGAGGCCGUUAGGCCGAA IUCCUCUC | 14749 |
| 6227 | GACAAAUC U UUGUUGUU | 5410 | AACAACAA CUGAUGAGGCCGUUAGGCCGAA IAUUUGUC | 14750 |
| 6237 | UGUUGUUC C UCUUCUUU | 5411 | AAAGAAGA CUGAUGAGGCCGUUAGGCCGAA IAACAACA | 14751 |
| 6238 | GUUGUUCC U CUUCUUUA | 5412 | UAAAGAAG CUGAUGAGGCCGUUAGGCCGAA IGAACAAC | 14752 |
| 6240 | UGUUCCUC U UCUUUACA | 5413 | UGUAAAGA CUGAUGAGGCCGUUAGGCCGAA IAGGAACA | 14753 |
| 6243 | UCCUCUUC U UUACACAU | 5414 | AUGUGUAA CUGAUGAGGCCGUUAGGCCGAA IAAGAGGA | 14754 |
| 6248 | UUCUUUAC A CAUACGCA | 5415 | UGCGUAUG CUGAUGAGGCCGUUAGGCCGAA IUAAAGAA | 14755 |
| 6250 | CUUUACAC A UACGCAAA | 5416 | UUUGCGUA CUGAUGAGGCCGUUAGGCCGAA IUGUAAAG | 14756 |
| 6256 | ACAUACGC A AACCACCU | 5417 | AGGUGGUU CUGAUGAGGCCGUUAGGCCGAA ICGUAUGU | 14757 |
| 6260 | ACGCAAAC C ACCUGUGA | 5418 | UCACAGGU CUGAUGAGGCCGUUAGGCCGAA IUUUGCGU | 14758 |
| 6261 | CGCAAACC A CCUGUGAC | 5419 | GUCACAUG CUGAUGAGGCCGUUAGGCCGAA IGUUUGCG | 14759 |
| 6263 | CAAACCAC C UGUGACAG | 5420 | CUGUCACA CUGAUGAGGCCGUUAGGCCGAA IUGGUUUG | 14760 |
| 6264 | AAACCACC U GUGACAGC | 5421 | GCUGUCAC CUGAUGAGGCCGUUAGGCCGAA IGUGGUGU | 14761 |
| 6270 | GCUGUGAC A GCUGGCAA | 5422 | UUGCCAGC CUGAUGAGGCCGUUAGGCCGAA IUCACAGG | 14762 |
| 6273 | GUGACAGC U GGCAAUUU | 5423 | AAAUUGCC CUGAUGAGGCCGUUAGGCCGAA ICUGUCAC | 14763 |
| 6277 | CAGCUGGC A AUUUUAUA | 5424 | UAUAAAAU CUGAUGAGGCCGUUAGGCCGAA ICCAGCUG | 14764 |
| 6290 | UAUAAAUC A GGUAACUG | 5425 | CAGUUACC CUGAUGAGGCCGUUAGGCCGAA IAUUUAUA | 14765 |
| 6297 | CAGGUAAC U GGAAGGAG | 5426 | CUCCUUCC CUGAUGAGGCCGUUAGGCCGAA IUUACCUG | 14766 |
| 6313 | GGUUAAAC U CAGAAAAA | 5427 | UUUUUCUG CUGAUGAGGCCGUUAGGCCGAA IUUUAACC | 14767 |
| 6315 | UUAAACUC A GAAAAAAG | 5428 | CUUUUUUC CUGAUGAGGCCGUUAGGCCGAA IAGUUUAA | 14768 |
| 6329 | AAGAAGAC C UCAGUCAA | 5429 | UUGACUGA CUGAUGAGGCCGUUAGGCCGAA IUCUUCUU | 14769 |
| 6330 | AGAAGACC U CAGUCAAU | 5430 | AGUGACUG CUGAUGAGGCCGUUAGGCCGAA IGUCUUCU | 14770 |
| 6332 | AAGACCUC A GUCAAUUC | 5431 | GAAUUGAC CUGAUGAGGCCGUUAGGCCGAA IAGGUCUU | 14771 |
| 6336 | CCUCAGUC A AUUCUCUA | 5432 | UAGAGAAU CUGAUGAGGCCGUUAGGCCGAA IACUGAGG | 14772 |
| 6341 | GUCAAUUC U CUACUUUU | 5433 | AAAAGUAG CUGAUGAGGCCGUUAGGCCGAA IAAUUGAC | 14773 |
| 6343 | CAAUUCUC U ACUUUUUU | 5434 | AAAAAAGU CUGAUGAGGCCGUUAGGCCGAA IAGAAUUG | 14774 |
| 6346 | UUCUCUAC U UUUUUUUU | 5435 | AAAAAAAA CUGAUGAGGCCGUUAGGCCGAA IUAGAGAA | 14775 |
| 6363 | UUUUUUUC C AAAUCAGA | 5436 | UCUGAUUU CUGAUGAGGCCGUUAGGCCGAA IAAAAAAA | 14776 |
| 6364 | UUUUUUCC A AAUCAGAU | 5437 | AUCUGAUU CUGAUGAGGCCGUUAGGCCGAA IGAAAAAA | 14777 |
| 6369 | UCCAAAUC A GAUAAUAG | 5438 | CUAUUAUC CUGAUGAGGCCGUUAGGCCGAA IAUUUGGA | 14778 |
| 6379 | AUAAUAGC C CAGCAAAU | 5439 | AUUUGCUG CUGAUGAGGCCGUUAGGCCGAA ICUAUUAU | 14779 |
| 6380 | UAAUAGCC C AGCAAAUA | 5440 | UAUUUGCU CUGAUGAGGCCGUUAGGCCGAA IGCUAUUA | 14780 |
| 6381 | AAUAGCCC A GCAAAUAG | 5441 | CUAUUUGC CUGAUGAGGCCGUUAGGCCGAA IGGCUAUU | 14781 |
| 6384 | AGCCCAGC A AAUAGUGA | 5442 | UCACUAUU CUGAUGAGGCCGUUAGGCCGAA ICUGGGCU | 14782 |
| 6397 | GUGAUAAC A AAUAAAAC | 5443 | GUUUUAUU CUGAUGAGGCCGUUAGGCCGAA IUUAUCAC | 14783 |
| 6406 | AAUAAAAC C UUAGCUGU | 5444 | ACAGCUAA CUGAUGAGGCCGUUAGGCCGAA IUUUUAUU | 14784 |
| 6407 | AUAAAACC U UAGCUGUU | 5445 | AACAGCUA CUGAUGAGGCCGUUAGGCCGAA IGUUUUAU | 14785 |
| 6412 | ACCUUAGC U GUUCAUGU | 5446 | ACAUGAAC CUGAUGAGGCCGUUAGGCCGAA ICUAAGGU | 14786 |
| 6417 | AGCUGUUC A UGUCUUGA | 5447 | UCAAGACA CUGAUGAGGCCGUUAGGCCGAA IAACAGCU | 14787 |
| 6422 | UUCAUGUC U UGAUUUCA | 5448 | UGAAAUCA CUGAUGAGGCCGUUAGGCCGAA IACAUGAA | 14788 |
| 6430 | UUGAUUUC A AUAAUUAA | 5449 | UUAAUUAU CUGAUGAGGCCGUUAGGCCGAA IAAAUCAA | 14789 |
| 6442 | AUUAAUUC U UAAUCAUU | 5450 | AAUGAUUA CUGAUGAGGCCGUUAGGCCGAA IAAUUAAU | 14790 |
| 6448 | UCUUAAUC A UUAAGAGA | 5451 | UCUCUUAA CUGAUGAGGCCGUUAGGCCGAA IAUUAAGA | 14791 |
| 6458 | UAAGAGAC C AUAAUAAA | 5452 | UUUAUUAU CUGAUGAGGCCGUUAGGCCGAA IUCUCUUA | 14792 |
| 6459 | AAGAGACC A UAAUAAAU | 5453 | AUUUAUUA CUGAUGAGGCCGUUAGGCCGAA IGUCUCUU | 14793 |
| 6470 | AUAAAUAC U CCUUUUCA | 5454 | UGAAAAGG CUGAUGAGGCCGUUAGGCCGAA IUAUUUAU | 14794 |
| 6472 | AAAUACUC C UUUUCAAG | 5455 | CUUGAAAA CUGAUGAGGCCGUUAGGCCGAA IAGUAUUU | 14795 |
| 6473 | AAUACUCC U UUUCAAGA | 5456 | UCUUGAAA CUGAUGAGGCCGUUAGGCCGAA IGAGUAUU | 14796 |
| 6478 | UCCUUUUC A AGAGAAAA | 5457 | UUUUCUCU CUGAUGAGGCCGUUAGGCCGAA IAAAAGGA | 14797 |
| 6489 | AGAAAAGC A AAACCAUU | 5458 | AAUGGUUU CUGAUGAGGCCGUUAGGCCGAA ICUUUUCU | 14798 |
| 6494 | AGCAAAAC C AUUAGAAU | 5459 | AUUCUAAU CUGAUGAGGCCGUUAGGCCGAA IUUUUGCU | 14799 |
| 6495 | GCAAAACC A UUAGAAUU | 5460 | AAUUCUAA CUGAUGAGGCCGUUAGGCCGAA IGUUUUGC | 14800 |
| 6509 | AUUGUUAC U CAGCUCCU | 5461 | AGGAGCUG CUGAUGAGGCCGUUAGGCCGAA IUAACAAU | 14801 |
| 6511 | UGUUACUC A GCUCCUUC | 5462 | GAAGGAGC CUGAUGAGGCCGUUAGGCCGAA IAGUAACA | 14802 |
| 6514 | UACUCAGC U CCUUCAAA | 5463 | UUUGAAGG CUGAUGAGGCCGUUAGGCCGAA ICUGAGUA | 14803 |
| 6516 | CUCACCUC C UUCAAACU | 5464 | ACUUUGAA CUGAUGAGGCCGUUAGGCCGAA IACCUGAG | 14804 |
| 6517 | UCAGCUCC U UCAAACUC | 5465 | GAGUUUGA CUGAUGAGGCCGUUAGGCCGAA IGACCUGA | 14805 |
| 6520 | GCUCCUUC A AACUCAGG | 5466 | CCUGAGUU CUGAUGAGGCCGUUAGGCCGAA IAAGGAGC | 14806 |
| 6524 | CUUCAAAC U CAGGUGUG | 5467 | CAAACCUG CUGAUGAGGCCGUUAGGCCGAA IUUUGAAG | 14807 |
| 6526 | UCAAACUC A GGUUUGUA | 5468 | UACAAACC CUGAUGAGGCCGUUAGGCCGAA IAGGUGGA | 14808 |
| 6537 | UUUGUAGC A GACAUGAG | 5469 | CUCAUGUA CUGAUGAGGCCGUUAGGCCGAA ICUACAAA | 14809 |
| 6541 | UAGCAUAC A UGAGUCCA | 5470 | UGGACUCA CUGAUGAGGCCGUUAGGCCGAA IUAUGCUA | 14810 |
| 6548 | CAUGAGUC C AUCCAUCA | 5471 | UGAUGGAU CUGAUGAGGCCGUUAGGCCGAA IACUCAUG | 14811 |
| 6549 | AUGAGUCC A UCCAUCAG | 5472 | CUGAUGGA CUGAUGAGGCCGUUAGGCCGAA IGACUCAU | 14812 |
| 6552 | AGUCCAUC C AUCAGUCA | 5473 | UGACUGAU CUGAUGAGGCCGUUAGGCCGAA IAUGGACU | 14813 |
| 6553 | GUCCAUCC A UCAGUCAA | 5474 | UUGACUGA CUGAUGAGGCCGUUAGGCCGAA IGAUGGAC | 14814 |
| 6556 | CAUCCAUC A GUCAAAGA | 5475 | UCUGUGAC CUGAUGAGGCCGUUAGGCCGAA IAUGGAUG | 14815 |
| 6560 | CAUCAGUC A AAGAAUGG | 5476 | CCAUGCUU CUGAUGAGGCCGUUAGGCCGAA IACUGAUG | 14816 |
| 6572 | AAUGGUUC C AUCUGGAG | 5477 | CUCCAGAU CUGAUGAGGCCGUUAGGCCGAA IAACCAUU | 14817 |
| 6573 | AUGGUUCC A UCUGGAGU | 5478 | ACUCCAGA CUGAUGAGGCCGUUAGGCCGAA IGAACCAU | 14818 |
| 6576 | GUUCCAUC U GGAGUCUU | 5479 | AAGACUCC CUGAUGAGGCCGUUAGGCCGAA IAUGGAAC | 14819 |
| 6583 | CUGGAGUC U UAAUGUAG | 5480 | CUACAUGA CUGAUGAGGCCGUUAGGCCGAA IACUCCAG | 14820 |
| 6608 | AUGGAGAC U UGUAAUAA | 5481 | UUAUUACA CUGAUGAGGCCGUUAGGCCGAA IUCUCCAU | 14821 |
| 6622 | UAAUGAGC U AGUUACAA | 5482 | UUGUAACU CUGAUGAGGCCGUUAGGCCGAA ICUCAUUA | 14822 |
| 6629 | CUAGUGAC A AAGUGCUU | 5483 | AAGCACUU CUGAUGAGGCCGUUAGGCCGAA IUAACUAG | 14823 |
| 6636 | CAAAGUGC U UGUUCAUU | 5484 | AAUGAACA CUGAUGAGGCCGUUAGGCCGAA ICACUUUG | 14824 |
| 6642 | GCUUGUUC A UUAAAAUA | 5485 | UAUUUUAA CUGAUGAGGCCGUUAGGCCGAA IAACAAGC | 14825 |
| 6653 | AAAAUAGC A CUGAAAAU | 5486 | AUUUUCAG CUGAUGAGGCCGUUAGGCCGAA ICUAUUUU | 14825 |
| 6655 | AAUAGCAC U GAAAAUUG | 5487 | CAAUUUUC CUGAUGAGGCCGUUAGGCCGAA IUGCUAUU | 14827 |
| 6668 | AUUGAAAC A UGAAUUAA | 5488 | UUAAUUCA CUGAUGAGGCCGUUAGGCCGAA IUUUCAAU | 14828 |
| 6678 | GAAUUAAC U GAUAAUAU | 5489 | AUAUUAUC CUGAUGAGGCCGUUAGGCCGAA IUUAAUUC | 14829 |
| 6689 | UAAUAUUC C AAUCAUUU | 5490 | AAAUGAUU CUGAUGAGGCCGUUAGGCCGAA IAAUAUUA | 14830 |
| 6690 | AAUAUUCC A AUCAUUUG | 5491 | CAAAUGAU CUGAUGAGGCCGUUAGGCCGAA IGAAUAUU | 14831 |
| 6694 | UUCCAAUC A UUUGCCAU | 5492 | AUGGCAAA CUGAUGAGGCCGUUAGGCCGAA IAUUGGAA | 14832 |
| 6700 | UCAUUUGC C AUGGAUGA | 5493 | UCAUAAAU CUGAUGAGGCCGUUAGGCCGAA ICAAAUGA | 14833 |
| 6701 | CAUUUGCC A UUUAUGAC | 5494 | GUCAUAAA CUGAUGAGGCCGUUAGGCCGAA IGCAAAUG | 14834 |
| 6710 | UUUAUGAC A AAAAUGGU | 5495 | ACCAUUUU CUGAUGAGGCCGUUAGGCCGAA IUCAUAAA | 14835 |
| 6723 | UGGUUGGC A CUAACAAA | 5496 | UGUGUGAG CUGAUGAGGCCGUUAGGCCGAA ICCAACCA | 14836 |
| 6725 | GUUGGCAC U AACAAAGA | 5497 | UCUUUGUU CUGAUGAGGCCGUUAGGCCGAA IUGCCAAC | 14837 |
| 6729 | GCACUAAC A AAGAACGA | 5498 | UCGUUCUU CUGAUGAGGCCGUUAGGCCGAA IUUAGUGC | 14838 |
| 6740 | GAACGAGC A CUUCCUUU | 5499 | AAAGGAAG CUGAUGAGGCCGUUAGGCCGAA ICUCGUUC | 14839 |
| 6742 | ACGAGCAC U UCCUUUCA | 5500 | UGAAAGGA CUGAUGAGGCCGUUAGGCCGAA IUGCUCGU | 14840 |
| 6745 | AGCACUUC C UUUCAGAG | 5501 | CUCUGAAA CUGAUGAGGCCGUUAGGCCGAA IAAGUGCU | 14841 |
| 6746 | GCACUUCC U UUCAGAGU | 5502 | ACUCUGAA CUGAUGAGGCCGUUAGGCCGAA IGAAGUGC | 14842 |
| 6750 | UUCCUUUC A GAGUGUCU | 5503 | AGAAACUC CUGAUGAGGCCGUUAGGCCGAA IAAAGGAA | 14843 |
| 6758 | AGAGUGUC U GAGAUAAU | 5504 | AUGAUCUC CUGAUGAGGCCGUUAGGCCGAA IAAACUCU | 14844 |
| 6778 | CGUGGAAC A GUCUGGGU | 5505 | ACCCAGAC CUGAUGAGGCCGUUAGGCCGAA IUUCCACG | 14845 |
| 6782 | GAACAGUC U GGGUGGAA | 5506 | UUCCACCC CUGAUGAGGCCGUUAGGCCGAA IACUGUUC | 14846 |
| 6797 | AAUGGGGC U GAAACCAU | 5507 | AUGGUUUC CUGAUGAGGCCGUUAGGCCGAA ICCCCAUU | 14847 |
| 6803 | GCUGAAAC C AUGUGCAA | 5508 | UUGCACAU CUGAUGAGGCCGUUAGGCCGAA IUUUCAGC | 14848 |
| 6804 | CUGAAACC A UGUGCAAG | 5509 | CUUGCACA CUGAUGAGGCCGUUAGGCCGAA IGUGUCAG | 14849 |
| 6810 | CCAUGUGC A AGUCUGUG | 5510 | CACAGACU CUGAUGAGGCCGUUAGGCCGAA ICACAUGG | 14850 |
| 6815 | UGCAAGUC U GUGUCUUG | 5511 | CAAGACAC CUGAUGAGGCCGUUAGGCCGAA IACUGGCA | 14851 |
| 6821 | UCUGUGUC U UGUCAGUC | 5512 | GACUGACA CUGAUGAGGCCGUUAGGCCGAA IACACAGA | 14852 |
| 6826 | GUCUUGUC A GUCCAAGA | 5513 | UCUUGGAC CUGAUGAGGCCGUUAGGCCGAA IACAAGAC | 14853 |
| 6830 | UGUCAGUC C AAGAAGUG | 5514 | CACUUCUU CUGAUGAGGCCGUUAGGCCGAA IACUGACA | 14854 |
| 6831 | GUCAGUCC A ACAAGUGA | 5515 | UCACUUCU CUGAUGAGGCCGUUAGGCCGAA IGACUGAC | 14855 |
| 6841 | GAAGUGAC A CCGAGAUG | 5516 | CAUCUCGG CUGAUGAGGCCGUUAGGCCGAA IUCACUUC | 14856 |
| 6843 | AGUGACAC C GAGAUGUU | 5517 | AACAUCUC CUGAUGAGGCCGUUAGGCCGAA IUGUCACU | 14857 |
| 6864 | UUAGGGAC C CGUGCCUU | 5518 | AAGGCACG CUGAUGAGGCCGUUAGGCCGAA IUCCCUAA | 14858 |
| 6865 | UAGGGACC C GUGCCUUG | 5519 | CAAGGCAC CUGAUGAGGCCGUUAGGCCGAA IGUCCCUA | 14859 |
| 6870 | ACCCGUGC C UUGUUUCC | 5520 | GGAAACAA CUGAUGAGGCCGUUAGGCCGAA ICACGGGU | 14860 |
| 6871 | CCCGUGCC U UGUUUCCU | 5521 | AGGAAACA CUGAUGAGGCCGUUAGGCCGAA IGCACGGG | 14861 |
| 6878 | CUUGUUUC C UAGCCCAC | 5522 | GUGGGCUA CUGAUGAGGCCGUUAGGCCGAA IAAACAAG | 14862 |
| 6879 | UUGUUUCC U AGCCCACA | 5523 | UGUGGGCU CUGAUGAGGCCGUUAGGCCGAA IGAAACAA | 14863 |
| 6883 | UUCCUAGC C CACAAGAA | 5524 | UUCUUGUG CUGAUGAGGCCGUUAGGCCGAA ICUAGGAA | 14864 |
| 6884 | UCCUAGCC C ACAAGAAU | 5525 | AUUCUUGU CUGAUGAGGCCGUUAGGCCGAA ICCUAGGA | 14865 |
| 6885 | CCUAGCCC A CAAGAAUG | 5526 | CAUUCUUG CUGAUGAGGCCGUUAGGCCGAA IGGCUAGG | 14866 |
| 6887 | UAGCCCAC A AGAAUGCA | 5527 | UGCAUUCU CUGAUGAGGCCGUUAGGCCGAA IUGGGCUA | 14867 |
| 6895 | AAGAAUGC A AACAUCAA | 5528 | UUGAUGUU CUGAUGAGGCCGUUAGGCCGAA ICAUUCUU | 14868 |
| 6899 | AUGCAAAC A UCAAACAG | 5529 | CUGUUUGA CUGAUGAGGCCGUUAGGCCGAA IUUUGCAU | 14869 |
| 6902 | CAAACAUC A AACAGAUA | 5530 | UAUCUGUU CUGAUGAGGCCGUUAGGCCGAA IAUGUUUG | 14870 |
| 6906 | CAUCAAAC A GAUACUCG | 5531 | CGAGUAUC CUGAUGAGGCCGUUAGGCCGAA IUUUGAUG | 14871 |
| 6912 | ACAGAUAC U CGCUAGCC | 5532 | GGCUAGCG CUGAUGAGGCCGUUAGGCCGAA IUAUCUGU | 14872 |
| 6916 | AUACUCGC U AGCCUCAU | 5533 | AUGAGGCU CUGAUGAGGCCGUUAGGCCGAA ICGAGUAU | 14873 |
| 6920 | UCGCUAGC C UCAUUUAA | 5534 | UUAAAUGA CUGAUGAGGCCGUUAGGCCGAA ICUAGCGA | 14874 |
| 6921 | CGCUAGCC U CAUUUAAA | 5535 | UUUAAAUG CUGAUGAGGCCGUUAGGCCGAA IGCUAGCG | 14875 |
| 6923 | CUAGCCUC A UUUAAAUU | 5536 | AAUUUAAA CUGAUGAGGCCGUUAGGCCGAA IAGGCUAG | 14876 |
| 6949 | AGGAGUGC A UCUUUGGC | 5537 | GCCAAAGA CUGAUGAGGCCGUUAGGCCGAA ICACUCCU | 14877 |
| 6952 | AGUGCAUC U UUGGCCGA | 5538 | UCGGCCAA CUGAUGAGGCCGUUAGGCCGAA IAUGCACU | 14878 |
| 6958 | UCUUUGGC C GACAGUGG | 5539 | CCACUGUC CUGAUGAGGCCGUUAGGCCGAA ICCAAAGA | 14879 |
| 6962 | UGGCCGAC A GUGGUGUA | 5540 | UACACCAC CUGAUGAGGCCGUUAGGCCGAA IUCGGCCA | 14880 |
| 6973 | GGUGUAAC U GUGUGUGU | 5541 | ACACACAC CUGAUGAGGCCGUUAGGCCGAA IUUACACC | 14881 |
| 7041 | UUUUGUGC A UAACUAUU | 5542 | AAUAGUUA CUGAUGAGGCCGUUAGGCCGAA ICACAAAA | 14882 |
| 7046 | UGCAUAAC U AUUUAAGG | 5543 | CCUUAAAU CUGAUGAGGCCGUUAGGCCGAA IUUAUGCA | 14883 |
| 7059 | AAGGAAAC U GGAAUUUU | 5544 | AAAAUUCC CUGAUGAGGCCGUUAGGCCGAA IUUUCCUU | 14884 |
| 7076 | AAAGUUAC U UUUAUACA | 5545 | UGUAUAAA CUGAUGAGGCCGUUAGGCCGAA IUAACUUU | 14885 |
| 7084 | UUUUAUAC A AACCAAGA | 5546 | UCUUGGUU CUGAUGAGGCCGUUAGGCCGAA IUAUAAAA | 14886 |
| 7088 | AUACAAAC C AAGAAUAU | 5547 | AUAUUCUU CUGAUGAGGCCGUUAGGCCGAA IUUUGUAU | 14887 |
| 7089 | UACAAACC A AGAAUAUA | 5548 | UAUAUUCU CUGAUGAGGCCGUUAGGCCGAA IGUUUGUA | 14888 |
| 7101 | AUAUAUGC U ACAGAUAU | 5549 | AUAUCUGU CUGAUGAGGCCGUUAGGCCGAA ICAUAUAU | 14889 |
| 7104 | UAUGCUAC A GAUAUAAG | 5550 | CUUAUAUC CUGAUGAGGCCGUUAGGCCGAA IUAGCAUA | 14890 |
| 7115 | UAUAAGAC A GACAUGGU | 5551 | ACCAUGUC CUGAUGAGGCCGUUAGGCCGAA IUCUUAUA | 14891 |
| 7119 | AGACAGAC A UGGUUUGG | 5552 | CCAAACCA CUGAUGAGGCCGUUAGGCCGAA IUCUGUCU | 14892 |
| 7130 | GUUUGGUC C UAUAUUUC | 5553 | GAAAUAUA CUGAUGAGGCCGUUAGGCCGAA IACCAAAC | 14893 |
| 7131 | UUUGGUCC U AUAUUUCU | 5554 | AGAAAUAU CUGAUGAGGCCGUUAGGCCGAA IGACCAAA | 14894 |
| 7139 | UAUAUUUC U AGUCAUGA | 5555 | UCAUGACU CUGAUGAGGCCGUUAGGCCGAA IAAAUAUA | 14895 |
| 7144 | UUCUAGUC A UGAUGAAU | 5556 | AUUCAUCA CUGAUGAGGCCGUUAGGCCGAA IACUAGAA | 14896 |
| 7166 | UUGUAUAC C AUCUUCAU | 5557 | AUGAAGAU CUGAUGAGGCCGUUAGGCCGAA IUAUACAA | 14897 |
| 7167 | UGUAUACC A UCUUCAUA | 5558 | UAUGAAGA CUGAUGAGGCCGUUAGGCCGAA IGUAUACA | 14898 |
| 7170 | AUACCAUC U UCAUAUAA | 5559 | UUAUAUGA CUGAUGAGGCCGUUAGGCCGAA IAUGGUAU | 14899 |
| 7173 | CCAUCUUC A UAUAAUAU | 5560 | AUAUUAUA CUGAUGAGGCCGUUAGGCCGAA IAAGAUGG | 14900 |
| 7184 | UAAUAUAC U UAAAAAUA | 5561 | UAUUUUUA CUGAUGAGGCCGUUAGGCCGAA IUAUAUUA | 14901 |
| 7197 | AAUAUUUC U UAAUUGGG | 5562 | CCCAAUUA CUGAUGAGGCCGUUAGGCCGAA IAAAUAUU | 14902 |
| 7220 | AAUCGUAC C AACUUAAU | 5563 | AUUAAGUU CUGAUGAGGCCGUUAGGCCGAA IUACGAUU | 14903 |
| 7221 | AUCGUACC A ACUUAAUU | 5564 | AAUUAAGU CUGAUGAGGCCGUUAGGCCGAA IGUACGAU | 14904 |
| 7224 | GUACCAAC U UAAUUGAU | 5565 | AUCAAUUA CUGAUGAGGCCGUUAGGCCGAA IUUGGUAC | 14905 |
| 7237 | UGAUAAAC U UGGCAACU | 5566 | AGUUGCCA CUGAUGAGGCCGUUAGGCCGAA IUUUAUCA | 14906 |
| 7242 | AACUUGGC A ACUGCUUU | 5567 | AAAGCAGU CUGAUGAGGCCGUUAGGCCGAA ICCAAGUU | 14907 |
| 7245 | UUGGCAAC U GCUUUUAU | 5568 | AUAAAAGC CUGAUGAGGCCGUUAGGCCGAA IUUGCCAA | 14908 |
| 7248 | GCAACUGC U UUUAUGUU | 5569 | AACAUAAA CUGAUGAGGCCGUUAGGCCGAA ICAGUUGC | 14909 |
| 7258 | UUAUGUUC U GUCUCCUU | 5570 | AAGGAGAC CUGAUGAGGCCGUUAGGCCGAA IAACAUAA | 14910 |
| 7262 | GUUCUCUC U CCUUCCAU | 5571 | AUGGAAGG CUGAUGAGGCCGUUAGGCCGAA IACAGAAC | 14911 |
| 7264 | UCUGUCUC C UUCCAUAA | 5572 | UUAUGGAA CUGAUGAGGCCGUUAGGCCGAA IAGACAGA | 14912 |
| 7265 | CUGUCUCC U UCCAUAAA | 5573 | UUUAUGGA CUGAUGAGGCCGUUAGGCCGAA IGAGACAG | 14913 |
| 7268 | UCUCCUUC C AUAAAUUU | 5574 | AAAUUUAU CUGAUGAGGCCGUUAGGCCGAA IAAGGAGA | 14914 |
| 7269 | CUCCUUCC A UAAAUUUU | 5575 | AAAAUUUA CUGAUGAGGCCGUUAGGCCGAA IGAAGGAG | 14915 |
| 7280 | AAUUUUUC A AAAUACUA | 5576 | UAGUAUUU CUGAUGAGGCCGUUAGGCCGAA IAAAAAUU | 14916 |
| 7287 | CAAAAUAC U AAUUCAAC | 5577 | GUUGAAUU CUGAUGAGGCCGUUAGGCCGAA IUAUUUUG | 14917 |
| 7293 | ACUAAUUC A ACAAAGAA | 5578 | UUCUUUGU CUGAUGAGGCCGUUAGGCCGAA IAAUUAGU | 14918 |
| 7296 | AAUUCAAC A AAGAAAAA | 5579 | UUUUUCUU CUGAUGAGGCCGUUAGGCCGAA IUUGAAUU | 14919 |
| 7307 | GAAAAAGC U CUUUUUUU | 5580 | AAAAAAAG CUGAUGAGGCCGUUAGGCCGAA ICUUUUUC | 14920 |
| 7309 | AAAAGCUC U UUUUUUUC | 5581 | GAAAAAAA CUGAUGAGGCCGUUAGGCCGAA IAGCUUUU | 14921 |
| 7318 | UUUUUUUC C UAAAAUAA | 5582 | UUAUUUUA CUGAUGAGGCCGUUAGGCCGAA IAAAAAAA | 14922 |
| 7319 | UUUUUUCC U AAAAUAAA | 5583 | UUUAUUUU CUGAUGAGGCCGUUAGGCCGAA IGAAAAAA | 14923 |
| 7329 | AAAUAAAC U CAAAUUUA | 5584 | UAAAUUUG CUGAUGAGGCCGUUAGGCCGAA IUUUAUUU | 14924 |
| 7331 | AUAAACUC A AAUUUAUC | 5585 | GAUAAAUU CUGAUGAGGCCGUUAGGCCGAA IAGUUUAU | 14925 |
| 7340 | AAUUUAUC C UUGUUUAG | 5586 | CUAAACAA CUGAUGAGGCCGUUAGGCCGAA IAUAAAUU | 14926 |
| 7341 | AUUUAUCC U UGUUUAGA | 5587 | UCUAAACA CUGAUGAGGCCGUUAGGCCGAA IGAUAAAU | 14927 |
| 7352 | UUUAGAGC A GAGAAAAA | 5588 | UUUUUCUC CUGAUGAGGCCGUUAGGCCGAA ICUCUAAA | 14928 |
| 7372 | AGAAAAAC U UUGAAAUG | 5589 | CAUUUCAA CUGAUGAGGCCGUUAGGCCGAA IUUUUUCU | 14929 |
| 7384 | AAAUGGUC U CAAAAAAU | 5590 | AUUUUUUG CUGAUGAGGCCGUUAGGCCGAA IACCAUUU | 14930 |
| 7386 | AUGGUCUC A AAAAAUUG | 5591 | CAAUUUUU CUGAUGAGGCCGUUAGGCCGAA IAGACCAU | 14931 |
| 7396 | AAAAUUGC U AAAUAUUU | 5592 | AAAUAUUU CUGAUGAGGCCGUUAGGCCGAA ICAAU1IUU | 14932 |
| 7407 | AUAUUUUC A AUGGAAAA | 5593 | UUUUCCAU CUGAUGAGGCCGUUAGGCCGAA IAAAAUAU | 14933 |
| 7417 | UGGAAAAC U AAAUGUUA | 5594 | UAACAUUU CUGAUGAGGCCGUUAGGCCGAA IUUUUCCA | 14934 |
| 7433 | AGUUUAGC U GAUUGUAU | 5595 | AUACAAUC CUGAUGAGGCCGUUAGGCCGAA ICUAAACU | 14935 |
| 7455 | UUUCGAAC C UUUCACUU | 5596 | AAGUGAAA CUGAUGAGGCCGUUAGGCCGAA IUUCGAAA | 14936 |
| 7456 | UUCGAACC U UUCACUUU | 5597 | AAAGUGAA CUGAUGAGGCCGUUAGGCCGAA IGUUCGAA | 14937 |
| 7460 | AACCUUUC A CUUUUUGU | 5598 | ACAAAAAG CUGAUGAGGCCGUUAGGCCGAA IAAAGGUU | 14938 |
| 7462 | CCUUUCAC U UUUUGUUU | 5599 | AAACAAAA CUGAUGAGGCCGUUAGGCCGAA IUGAAAGG | 14939 |
| 7478 | UGUUUUAC C UAUUUCAC | 5600 | GUGAAAUA CUGAUGAGGCCGUUAGGCCGAA IUAAAACA | 14940 |
| 7479 | GUUUUACC U AUUUCACA | 5601 | UGUGAAAU CUGAUGAGGCCGUUAGGCCGAA IGUAAAAC | 14941 |
| 7485 | CCUAUUUC A CAACUGUG | 5602 | CACAGUUG CUGAUGAGGCCGUUAGGCCGAA IAAAUAGG | 14942 |
| 7487 | UAUUUCAC A ACUGUGUA | 5603 | UACACAGU CUGAUGAGGCCGUUAGGCCGAA IUGAAAUA | 14943 |
| 7490 | UUCACAAC U GUGUAAAU | 5604 | AUUUACAC CUGAUGAGGCCGUUAGGCCGAA IUUGUGAA | 14944 |
| 7502 | UAAAUUGC C AAUAAUUC | 5605 | GAAUUAUU CUGAUGAGGCCGUUAGGCCGAA ICAAUUUA | 14945 |
| 7503 | AAAUUGCC A AUAAUUCC | 5606 | GGAAUUAU CUGAUGAGGCCGUUAGGCCGAA IGCAAUUU | 14946 |
| 7511 | AAUAAUUC C UGUCCAUG | 5607 | CAUGGACA CUGAUGAGGCCGUUAGGCCGAA IAAUUAUU | 14947 |
| 7512 | AUAAUUCC U GUCCAUGA | 5608 | UCAUGGAC CUGAUGAGGCCGUUAGGCCGAA IGAAUUAU | 14948 |
| 7516 | UUCCUGUC C AUGAAAAU | 5609 | AUUUUCAU CUGAUGAGGCCGUUAGGCCGAA IACAGGAA | 14949 |
| 7517 | UCCUGUCC A UGAAAAUG | 5610 | CAUUUUCA CUGAUGAGGCCGUUAGGCCGAA IGACAGGA | 14950 |
| 7527 | GAAAAUGC A AAUUAUCC | 5611 | GGAUAAUU CUGAUGAGGCCGUUAGGCCGAA ICAUUUUC | 14951 |
| 7535 | AAAUUAUC C AGUGUAGA | 5612 | UCUACACU CUGAUGAGGCCGUUAGGCCGAA IAUAAUUU | 14952 |
| 7536 | AAUUAUCC A GUGUAGAU | 5613 | AUCUACAC CUGAUGAGGCCGUUAGGCCGAA IGAUAAUU | 14953 |
| 7554 | UAUUUGAC C AUCACCCU | 5614 | AGGGUGAU CUGAUGAGGCCGUUAGGCCGAA IUCAAAUA | 14954 |
| 7555 | AUUUGACC A UCACCCUA | 5615 | UAGGGUGA CUGAUGAGGCCGUUAGGCCGAA IGUCAAAU | 14955 |
| 7558 | UGACCAUC A CCCUAUGG | 5616 | CCAUAGGG CUGAUGAGGCCGUUAGGCCGAA IAUGGUCA | 14956 |
| 7560 | ACCAUCAC C CUAUCGAU | 5617 | AUCCAUAG CUGAUGAGGCCGUUAGGCCGAA IUGAUGGU | 14957 |
| 7561 | CCAUCACC C UAUGGAUA | 5618 | UAUCCAUA CUGAUGAGGCCGUUAGGCCGAA ICUGAUGG | 14958 |
| 7562 | CAUCACCC U AUGGAUAU | 5619 | AUAUCCAU CUGAUGAGGCCGUUAGGCCGAA IGGUGAUG | 14959 |
| 7575 | AUAUUGGC U AGUUUUGC | 5620 | GCAAAACU CUGAUGAGGCCGUUAGGCCGAA ICCAAUAU | 14960 |
| 7584 | AGUUUUGC C UUUAUUAA | 5621 | UUAAUAAA CUGAUGAGGCCGUUAGGCCGAA ICAAAACU | 14961 |
| 7585 | GUUUUGCC U UUAUUAAG | 5622 | CUUAAUAA CUGAUGAGGCCGUUAGGCCGAA IGCAAAAC | 14962 |
| 7595 | UAUUAAGC A AAUUCAUU | 5623 | AAUGAAUU CUGAUGAGGCCGUUAGGCCGAA ICUUAAUA | 14963 |
| 7601 | GCAAAUUC A UUUCAGCC | 5624 | GGCUGAAA CUGAUGAGGCCGUUAGGCCGAA IAAUUUGC | 14964 |
| 7606 | UUCAUUUC A GCCUGAAU | 5625 | AUUCAGGC CUGAUGAGGCCGUUAGGCCGAA IAAAUGAA | 14965 |
| 7609 | AUUUCAGC C UGAAUGUC | 5626 | GACAUUCA CUGAUGAGGCCGUUAGGCCGAA ICUGAAAU | 14966 |
| 7610 | UUUCAGCC U GAAUGUCU | 5627 | AGACAUUC CUGAUGAGGCCGUUAGGCCGAA IGCUGAAA | 14967 |
| 7618 | UGAAUGUC U GCCUAUAU | 5628 | AUAUAGGC CUGAUGAGGCCGUUAGGCCGAA IACAUUCA | 14968 |
| 7621 | AUGUCUGC C UAUAUAUU | 5629 | AAUAUAUA CUGAUGAGGCCGUUAGGCCGAA ICAGACAU | 14969 |
| 7622 | UGUCUGCC U AUAUAUUC | 5630 | GAAUAUAU CUGAUGAGGCCGUUAGGCCGAA IGCAGACA | 14970 |
| 7631 | AUAUAUUC U CUGCUCUU | 5631 | AAGAGCAG CUGAUGAGGCCGUUAGGCCGAA IAAUAUAU | 14971 |
| 7633 | AUAUUCUC U GCUCUUUG | 5632 | CAAAGAGC CUGAUGAGGCCGUUAGGCCGAA IAGAAUAU | 14972 |
| 7636 | UUCUCUGC U CUUUGUAU | 5633 | AUACAAAG CUGAUGAGGCCGUUAGGCCGAA ICAGAGAA | 14973 |
| 7638 | CUCUGCUC U UUGUAUUC | 5634 | GAAUACAA CUGAUGAGGCCGUUAGGCCGAA IAUCAGAG | 14974 |
| 7647 | UUGUAUUC U CCUUUGAA | 5635 | UUCAAAGG CUGAUGAGGCCGUUAGGCCGAA IAAUACAA | 14975 |
| 7649 | GUAUUCUC C UUUGAACC | 5636 | GGUUCAAA CUGAUGAGGCCGUUAGGCCGAA IAGAAUAC | 14976 |
| 7650 | UAUUCUCC U UUGAACCC | 5637 | GGGUUCAA CUGAUGAGGCCGUUAGGCCGAA IGAGAAUA | 14977 |
| 7657 | CUUUGAAC C CGUUAAAA | 5638 | UUUUAACG CUGAUGAGGCCGUUAGGCCGAA IUUCAAAG | 14978 |
| 7658 | UUUGAACC C GUUAAAAC | 5639 | GUUUUAAC CUGAUGAGGCCGUUAGGCCGAA IGUUCAAA | 14979 |
| 7667 | GUUAAAAC A UCCUGUGG | 5640 | CCACAGGA CUGAUGAGGCCGUUAGGCCGAA IUUUUAAC | 14980 |
| 7670 | AAAACAUC C UGUGUCAC | 5641 | GUGCCACA CUGAUGAGGCCGUUAGGCCGAA IAUGUUUU | 14981 |
| 7671 | AAACAUCC U GUGGCACU | 5642 | AGUGCCAC CUGAUGAGGCCGUUAGGCCGAA IGAUGUUU | 14982 |

Core sequence = CUGAUGAG GCCGUUAGGC CGAA.
Underlined region can be any X sequence or linker, as described herein.
“I” represents Inosine.

TABLE XV
G-Cleaver Ribozyme and Target Sequences

| | | Seq ID | | Seq ID |
|---|---|---|---|---|
| Pos | Target | No | Ribozyme Sequence | No |
| 67 | CGGCUCGGGU C CAGCG | 5643 | CCCUG UGAUG GCAUGCACUAUGC GCG ACCCGAGCCC | 14983 |
| 91 | GCCUGGCGGC G AGGAG | 5644 | AUCCU UGAUG GCAUGCACUAUGC GCG GCCGCCAGGC | 14984 |
| 114 | GGAAGUGGUU C UCUCC | 5645 | GCAGA UGAUG CCAUGCACUAUGC CCC AACCACUUCC | 14985 |
| 131 | CCCUCCACCC G CGACA | 5646 | UCUCG UGAUG CCAUGCACUAUCC GCC GCCUCCACCC | 14986 |
| 133 | CUCGAGCCCC G AGACC | 5647 | CCUCU UCAUC CCAUGCACUAUCC GCG GCCCCUCCAC | 14987 |
| 142 | CGAGACCGGC C CUCAG | 5648 | CUGAG UGAUG CCAUGCACUAUCC CCG GCCCGUCUCG | 14988 |
| 151 | CGCUCAGGGC G CGCGG | 5649 | CCCCC UCAUC GCAUCCACUAUCC CCC GCCCUCAGCC | 14989 |
| 168 | CGCCCGCGGC C AACCA | 5650 | UCGUU UGAUG CCAUGCACUAUGC GCC GCCCCCCCCC | 14990 |
| 172 | GGCGGCGAAC C AGAGG | 5651 | CCUCU UGAUC CCAUGCACUAUGC CCG GUUCCCCGCC | 14991 |
| 214 | CCCCGCCACC G CCCCC | 5652 | CCCCC UCAUG GCAUCCACUAUCC CCG GCUCCCCCGC | 14992 |
| 227 | CCCACCGCGC G ACCAC | 5653 | CUCCU UCAUC GCAUCCACUAUCC GCG GCCCGCUGCC | 14993 |
| 236 | CGAGCAGGCC C CCUCG | 5654 | CGACC UGAUG CCAUCCACUAUCC CCC CGCCUGCUCC | 14994 |
| 241 | AGGCCCCGUC C CCCUC | 5655 | CAGCC UCAUG CCAUGCACUAUGC GCC CACCCCCCCU | 14995 |
| 243 | GCCGCGUCGC C CUCAC | 5656 | GUCAC UGAUG CCAUCCACUAUCC GCG GCGACGCGCC | 14996 |
| 279 | CCGCCCUCCU G CUCUC | 5657 | CACAC UCAUC GCAUCCACUAUCC CCG ACGACCCCGC | 14997 |
| 282 | GCCUCCUCCU C UGCCC | 5658 | CCGCA UGAUC GCAUGCACUAUCC GCG AGCACCACCC | 14998 |
| 284 | GUCCUGCUGU G CCCCC | 5659 | CCCCC UCAUC GCAUGCACUAUCC GCC ACACCACCAC | 14999 |
| 286 | CCUGCUCUGC C CCCUG | 5660 | CAGCC UCAUG GCAUCCACUAUCC CCC CCACACCACC | 15000 |
| 288 | UCCUCUCCCC C CUCCU | 5661 | AGCAC UGAUG CCAUGCACUAUGC GCC GCCCACAGCA | 15001 |
| 291 | UCUGCGCCCU C CUCAC | 5662 | CUGAC UGAUG GCAUGCACUAUCC GCG AGCCCGCACA | 15002 |
| 299 | CUCCUCACCU C UCUCC | 5663 | GCAGA UCAUG CCAUCCACUAUCC CCC AGCUGACCAG | 15003 |
| 303 | UCACCUCUCU C CUUCU | 5664 | ACAAC UCAUC CCAUCCACUAUCC CCC ACACACCUCA | 15004 |
| 346 | AAAACAUCCU C AACUC | 5665 | CAGUU UCAUC CCAUCCACUAUCC CCC ACCAUCUUUU | 15005 |
| 351 | AUCCUGAACU C ACUUU | 5666 | AAACU UCAUC CCAUCCACUAUCC CCC ACUUCACCAU | 15006 |
| 378 | ACCACAUCAC C CAACC | 5667 | CCUUC UCAUC CCAUCCACUAUCC CCC AUGAUCUCCU | 15007 |
| 396 | CCCACACACU C CAUCU | 5668 | ACAUC UCAUC CCAUCCACUAUCC CCC ACUCUCUCCC | 15008 |
| 407 | CAUCUCCAAU C CACGC | 5669 | CCCUC UCAUC CCAUCCACUAUCC CCC AUUCCACAUC | 15009 |
| 438 | AAUCCUCUUU C GCUCA | 5670 | UCACC UCAUC CCAUCCACUAUCC CCC AAACACCAUU | 15010 |
| 442 | CUCUUUCCCU C AAAUC | 5671 | CAUUU UCAUC CCAUCCACUAUCC CCC ACCCAAACAC | 15011 |
| 450 | CUCAAAUCCU C ACUAA | 5672 | UUACU UCAUC CCAUCCACUAUCC CCC ACCAUUUCAC | 15012 |
| 463 | UAACCAAACC C AAACC | 5673 | CCUUU UCAUC CCAUCCACUAUCC CCC CCUUUCCUUA | 15013 |
| 471 | CCCAAACCCU C ACCAU | 5674 | AUCCU UCAUC CCAUCCACUAUCC CCC ACCCUUUCCC | 15014 |
| 487 | AACUAAAUCU C CCUCU | 5675 | ACACC UCAUC CCAUCCACUAUCC CCC ACAUUUACUU | 15015 |
| 491 | AAAUCUCCCU C UCCAA | 5676 | UUCCA UCAUC CCAUCCACUAUCC CCC ACCCACAUUU | 15016 |
| 515 | AAACAAUUCU C CACUA | 5677 | CACUC UCAUC CCAUCCACUAUCC CCC ACAAUUCUUU | 15017 |
| 531 | CUUUAACCUU C AACAC | 5678 | CUCUU UCAUC CCAUCCACUAUCC CCC AACCUUAAAC | 15018 |
| 569 | UUCUACACCU C CAAAU | 5679 | AUCUC UCAUC CCAUCCACUAUCC CCC ACCUCUACAA | 15019 |
| 583 | AUAUCUACCU C UACCU | 5680 | ACCUA UCAUC CCAUCCACUAUCC CCC ACCUACAUAU | 15020 |
| 616 | AACACAAUCU C CAAUC | 5681 | CACUC UCAUC CCAUCCACUAUCC CCC ACAUUCUCUU | 15021 |
| 637 | AUUUAUUACU C AUACA | 5682 | UCUAC UCAUC CCAUCCACUAUCC CCC ACUAAUAAAU | 15022 |
| 663 | UCCUACACAU C UACAC | 5683 | CUCUA UCAUC CCAUCCACUAUCC CCC AUCUCUACCA | 15023 |
| 670 | CAUCUACACU C AAAUC | 5684 | CAUUU UCAUC CCAUCCACUAUCC CCC ACUCUACAUC | 15024 |
| 679 | UCAAAUCCCC C AAAUU | 5685 | AAUUU UCAUC CCAUCCACUAUCC CCC CCCCAUUUCA | 15025 |
| 693 | UUAUACACAU C ACUCA | 5686 | UCACU UCAUC CCAUCCACUAUCC CCC AUCUCUAUAA | 15026 |
| 697 | ACACAUCACU C AACCA | 5687 | UCCUU UCAUC CCAUCCACUAUCC CCC ACUCAUCUCU | 15027 |
| 722 | GUCAUUCCCU G CCGGG | 5688 | CCCGG UGAUG GCAUGCACUAUGC GCG AGGGAAUGAC | 15028 |
| 748 | UAACAUCACU G UUACU | 5689 | AGUAA UGAUG GCAUGCACUAUGC GCG AGUGAUGUUA | 15029 |
| 772 | GUUUCCACUU G ACACU | 5690 | AGUGU UGAUG GCAUGCACUAUGC GCG AAGUGGAAAC | 15030 |
| 780 | UUGACACUUU G AUCCC | 5691 | GGGAU UGAUG GCAUGCACUAUGC GCG AAAGUGUCAA | 15031 |
| 787 | UUUGAUCCCU G AUGGA | 5692 | UCCAU UGAUG GCAUGCACUAUGC GCG AGGGAUCAAA | 15032 |
| 797 | GAUGGAAAAC G CAUAA | 5693 | UUAUG UGAUG GCAUGCACUAUGC GCG GUUUUCCAUC | 15033 |
| 838 | CAUAUCAAAU G CAACG | 5694 | CGUUG UGAUG GCAUGCACUAUGC GCG AUUUGAUAUG | 15034 |
| 864 | UAGGGCUUCU G ACCUG | 5695 | CAGGU UGAUG GCAUGCACUAUGC GCG AGAAGCCCUA | 15035 |
| 869 | CUUCUGACCU G UGAAG | 5696 | CUUCA UGAUG GCAUGCACUAUGC GCG AGGUCAGAAG | 15036 |
| 871 | UCUGACCUGU G AAGCA | 5697 | UGCUU UGAUG GCAUGCACUAUGC GCG ACAGGUCAGA | 15037 |
| 894 | AUGGGCAUUU G UAUAA | 5698 | UUAUA UGAUG GCAUGCACUAUGC GCG AAAUGCCCAU | 15038 |
| 920 | CUCACACAUC G ACAAA | 5699 | UUUGU UGAUG GCAUGCACUAUGC GCG GAUGUGUGAG | 15039 |
| 943 | AAUCAUAGAU G UCCAA | 5700 | UUGGA UGAUG GCAUGCACUAUGC GCG AUCUAUGAUU | 15040 |
| 962 | ACCACACCAC G CCCAG | 5701 | CUGGG UGAUG GCAUGCACUAUGC GCG GUGGUGUGCU | 15041 |
| 994 | CCAUACUCUU G UCCUC | 5702 | GAGGA UGAUG GCAUGCACUAUGC GCG AAGAGUAUGG | 15042 |
| 1004 | GUCCUCAAUU G UACUG | 5703 | CAGUA UGAUG GCAUGCACUAUGC GCG AAUUGAGGAC | 15043 |
| 1009 | CAAUUGUACU G CUACC | 5704 | GGUAG UGAUG GCAUGCACUAUGC GCG AGUACAAUUG | 15044 |
| 1023 | CCACUCCCUU G AACAC | 5705 | GUGUU UGAUG GCAUGCACUAUGC GCG AAGGGAGUGG | 15045 |
| 1029 | CCUUGAACAC G AGAGU | 5706 | ACUCU UGAUG GCAUGCACUAUGC GCG GUGUUCAAGG | 15046 |
| 1041 | GAGUUCAAAU G ACCUG | 5707 | CAGGU UGAUG GCAUGCACUAUGC GCG AUUUGAACUC | 15047 |
| 1057 | GAGUUACCCU G AUGAA | 5708 | UUCAU UGAUG GCAUGCACUAUGC GCG AGGGUAACUC | 15048 |
| 1060 | UUACCCUGAU G AAAAA | 5709 | UUUUU UGAUG GCAUGCACUAUGC GCG AUCAGGGUAA | 15049 |
| 1088 | UCCGUAAGGC G ACGAA | 5710 | UUCGU UGAUG GCAUGCACUAUGC GCG GCCUUACGGA | 15050 |
| 1091 | GUAAGGCGAC G AAUUG | 5711 | CAAUU UGAUG GCAUGCACUAUGC GCG GUCGCCUUAC | 15051 |
| 1096 | GCGACGAAUU G ACCAA | 5712 | UUGGU UGAUG GCAUGCACUAUGC GCG AAUUCGUCGC | 15052 |
| 1114 | CAAUUCCCAU G CCAAC | 5713 | GUUGG UGAUG GCAUGCACUAUGC GCG AUGGGAAUUG | 15053 |
| 1132 | AUUCUACAGU G UUCUU | 5714 | AAGAA UGAUG GCAUGCACUAUGC GCG ACUGUAGAAU | 15054 |
| 1144 | UCUUACUAUU G ACAAA | 5715 | UUUGU UGAUG GCAUGCACUAUGC GCG AAUAGUAAGA | 15055 |
| 1152 | UUGACAAAAU G CAGAA | 5716 | UUCUG UGAUG GCAUGCACUAUGC GCG AUUUUGUCAA | 15056 |
| 1181 | CUUUAUACUU G UCGUG | 5717 | CACGA UGAUG GCAUGCACUAUGC GCG AAGUAUAAAG | 15057 |
| 1186 | UACUUGUCGU G UAAGG | 5718 | CCUUA UGAUG GCAUGCACUAUGC GCG ACGACAAGUA | 15058 |
| 1213 | AUUCAAAUCU G UUAAC | 5719 | GUUAA UGAUG GCAUGCACUAUGC GCG AGAUUUGAAU | 15059 |
| 1227 | ACACCUCAGU G CAGAG | 5720 | AUAUG UGAUG GCAUGCACUAUGC GCG ACUGAGGUGU | 15060 |
| 1237 | GCAUAUAUAU G AUAAA | 5721 | UUUAU UGAUG GCAUGCACUAUGC GCG AUAUAUAUGC | 15061 |
| 1255 | AUUCAUCACU G UGAAU | 5722 | UUUCA UGAUG GCAUGCACUAUGC GCG AGUGAUGAAU | 15062 |
| 1257 | UCAUCACUGU G AAACA | 5723 | UGUUU UGAUG GCAUGCACUAUGC GCG ACAGUGAUGA | 15063 |
| 1265 | GUGAAACAUC G AAAAC | 5724 | GUUUU UGAUG GCAUGCACUAUGC GCG GAUGUUUCAC | 15064 |
| 1278 | AACAGCAGGU G CUUGA | 5725 | UCAAG UGAUG GCAUGCACUAUGC GCG ACCUGCUGUU | 15065 |
| 1282 | GCAGGUGCUU G AAACC | 5726 | GGUUU UGAUG GCAUGCACUAUGC GCG AAGCACCUGC | 15066 |
| 1320 | GGCUCUCUAU G AAAGU | 5727 | ACUUU UGAUG GCAUGCACUAUGC GCG AUAGAGAGCC | 15067 |
| 1326 | CUAUGAAAGU G AAGGC | 5728 | GCCUU UGAUG GCAUGCACUAUGC GCG ACUUUCAUAG | 15068 |
| 1341 | CAUUUCCCUC G CCGGA | 5729 | UCCGG UGAUG GCAUGCACUAUGC GCG GAGGGAAAUG | 15069 |
| 1351 | GCCGGAAGUU G UAUGG | 5730 | CCAUA UGAUG GCAUGCACUAUGC GCG AACUUCCGGC | 15070 |
| 1375 | UGGGUUACCU G CGACU | 5731 | AGUCG UGAUG GCAUGCACUAUGC GCG AGGUAACCCA | 15071 |
| 1377 | GGUUACCUGC G ACUGA | 5732 | UCAGU UGAUG GCAUGCACUAUGC GCG GCAGGUAACC | 15072 |
| 1381 | ACCUGCGACU G AGAAA | 5733 | UUUCU UGAUG GCAUGCACUAUGC GCG AGUCGCAGGU | 15073 |
| 1390 | UGAGAAAUCU G CUCGC | 5734 | GCGAG UGAUG GCAUGCACUAUGC GCG AGAUUUCUCA | 15074 |
| 1394 | AAAUCUGCUC G CUAUU | 5735 | AAUAG UGAUG GCAUGCACUAUGC GCG GAGCAGAUUU | 15075 |
| 1401 | CUCGCUAUUU G ACUCG | 5736 | CGAGU UGAUG GCAUGCACUAUGC GCG AAAUAGCGAG | 15076 |
| 1438 | GGACGUAACU G AAGAG | 5737 | CUCUU UGAUG GCAUGCACUAUGC GCG AGUUACGUCC | 15077 |
| 1447 | UGAAGAGGAU U CAGUG | 5738 | CCCUG UGAUG GCAUGCACUAUGC GCG AUCCUCUUCA | 15078 |
| 1467 | AUACAAUCUU C CUGAG | 5739 | CUCAG UGAUG GCAUGCACUAUGC GCG AAGAUUGUAU | 15079 |
| 1470 | CAAUCUUGCU G AGCAU | 5740 | AUGCU UGAUG GCAUGCACUAUGC GCG AGCAAGAUUG | 15080 |
| 1489 | ACAGUCAAAU C UGUUU | 5741 | AAACA UGAUG GCAUGCACUAUGC GCG AUUUGACUGU | 15081 |
| 1491 | AGUCAAAUGU G UUUAA | 5742 | UUAAA UGAUG GCAUGCACUAUGC GCG ACAUUUGACU | 15082 |
| 1507 | AAACCUCACU G CCACU | 5743 | AGUGG UGAUG GCAUGCACUAUGC GCG AGUGAGGUUU | 15083 |
| 1519 | CACUCUAAUU C UCAAU | 5744 | AGUGA UGAUG GCAUGCACUAUGC GCG AAUUAGAGUG | 15084 |
| 1525 | AAUUGUCAAU G UGAAA | 5745 | UUUCA UCAUG GCAUGCACUAUGC CCC AUUCACAAUU | 15085 |
| 1527 | UUCUCAAUGU G AAACC | 5746 | GGUUU UGAUG GCAUGCACUAUGC GCG ACAUUGACAA | 15086 |
| 1543 | CCAGAUUUAC G AAAAG | 5747 | CUUUU UGAUG GCAUCCACUAUGC GCG GUAAAUCUCG | 15087 |
| 1554 | AAAAGGCCGU C UCAUC | 5748 | GAUGA UGAUG GCAUGCACUAUCC GCC ACGCCCUUUU | 15088 |
| 1605 | GACAAAUCCU C ACUUG | 5749 | CAAGU UGAUC GCAUGCACUAUGC GCG AGGAUGUGUC | 15089 |
| 1610 | AUCCUGACUU G UACCC | 5750 | CGGUA UGAUG GCAUGCACUAUGC CCC AAGUCAGGAU | 15090 |
| 1615 | CACUUGUACC G CAUAU | 5751 | AUAUG UGAUG GCAUGCACUAUGC GCG GGUACAAGUC | 15091 |
| 1661 | UGGCACCCCU G UAACC | 5752 | GGUUA UGAUG GCAUGCACUAUGC GCG AGCGGUCCCA | 15092 |
| 1678 | UAAUCAUUCC G AACCA | 5753 | UGCUU UGAUG GCAUGCACUAUGC CCG GCAAUCAUUA | 15093 |
| 1688 | GAAGCAACGU G UGACU | 5754 | ACUCA UGAUG GCAUGCACUAUGC GCG ACCUUGCUUC | 15094 |
| 1690 | AGCAAGGUGU G ACUUU | 5755 | AAAGU UCAUG GCAUCCACUAUGC CCG ACACCUUGCU | 15095 |
| 1697 | UGUGACUUUU C GUCCA | 5756 | UGGAA UGAUG GCAUGCACUAUGC GCG AAAAGUCACA | 15096 |
| 1708 | UUCCAAUAAU C AAGAG | 5757 | CUCUU UCAUG GCAUGCACUAUGC GCC AUUAUUGGAA | 15097 |
| 1729 | UAUCCUGGAU G CUGAC | 5758 | GUCAG UGAUG GCAUGCACUAUGC GCG AUCCAGGAUA | 15098 |
| 1732 | CCUGGAUGCU C ACAUC | 5759 | GCUGU UGAUG GCAUGCACUAUGC GCG AGCAUCCAGG | 15099 |
| 1756 | AAACAGAAUU G AGAGC | 5760 | CCUCU UGAUC CCAUCCACUAUCC GCG AAUUCUGUGU | 15100 |
| 1772 | AUCACUCAUC C CAUCG | 5761 | CCAUG UCAUG GCAUGCACUAUGC GCC CCUCACUCAU | 15101 |
| 1819 | CACCUUGCUU C UGGCU | 5762 | AUCCA UCAUC GCAUGCACUAUCC GCG AACCAACCUG | 15102 |
| 1825 | CGUUGUGGCU G ACUCU | 5763 | AGAGU UGAUG GCAUGCACUAUCC CCC AGCCACAACC | 15103 |
| 1853 | AUCUACAUUU G CAUAC | 5764 | CUAUG UGAUG CCAUGCACUAUCC CCC AAAUGUAGAU | 15104 |
| 1879 | AGUUGCGACU C UGUGA | 5765 | UCCCA UCAUC CCAUCCACUAUGC GCG AGUCCCAACU | 15105 |
| 1912 | UAUCACAGAU G UCCCA | 5766 | UCUCA UGAUG GCAUGCACUAUCC GCC AUCUCUCAUA | 15106 |
| 1914 | UCACAGAUCU C CCAAA | 5767 | UUUCC UCAUG GCAUCCACUAUCC CCG ACAUCUGUGA | 15107 |
| 1930 | UGGCUUUCAU C UUAAC | 5768 | GUUAA UCAUC CCAUCCACUAUGC GCG AUCAAACCCA | 15108 |
| 1947 | UCCAAAAAAU G CCCAC | 5769 | CUCCC UGAUG GCAUGCACUAUGC CCC AUUUUUUCCA | 15109 |
| 1950 | AAAAAAUGCC C ACCCA | 5770 | UCCGU UGAUG GCAUCCACUAUCC CCC GGCAUUUUUU | 15110 |
| 1968 | GAGACCACCU C AAACU | 5771 | AGUUU UCAUC CCAUGCACUAUGC GCG AGGUCCUCUC | 15111 |
| 1974 | ACCUGAAACU U UCUUG | 5772 | CAACA UCAUG GCAUGCACUAUGC GCC AGUUUCACCU | 15112 |
| 1979 | AAACUGUCUU C CACAG | 5773 | CUCUG UGAUG GCAUCCACUAUGC CCC AACACACUUU | 15113 |
| 2025 | CGAUUUUACU C CGGAC | 5774 | CUCCG UCAUC GCAUCCACUAUGC CCC ACUAAAAUCC | 15114 |
| 2049 | ACACAACAAU C CACUA | 5775 | UACUG UCAUG GCAUCCACUAUGC GCG AUUCUUCUGU | 15115 |
| 2121 | UUACCAUCAU G AAUCU | 5776 | ACAUU UCAUG GCAUCCACUAUGC GCG AUCAUGGUAA | 15116 |
| 2125 | CAUCAUGAAU C UUUCC | 5777 | CGAAA UGAUC GCAUCCACUAUGC CCC AUUCAUGAUC | 15117 |
| 2133 | AUGUUUCCCU C CAACA | 5778 | UCUUC UGAUG GCAUCCACUAUGC CCC ACCCAAACAU | 15118 |
| 2152 | ACCCACCUAU G CCUCC | 5779 | UCAUC UCAUG CCAUCCACUAUGC CCC AUAGGUGCCU | 15119 |
| 2156 | ACCUAUGCCU U CACAG | 5780 | CUCUC UCAUC CCAUCCACUAUGC GCG AGGCAUAGGU | 15120 |
| 2170 | AGCCAGGAAU C UAUAC | 5781 | CUAUA UGAUG GCAUGCACUAUGC CCC AUUCCUCGCU | 15121 |
| 2241 | CAUACCUCCU U CGAAA | 5782 | UUUCG UGAUC GCAUCCACUAUGC CCC ACCAGGUAUG | 15122 |
| 2243 | UACCUCCUGC G AAACC | 5783 | CCUUU UCAUC CCAUCCACUAUGC CCC CCACCAGCUA | 15123 |
| 2254 | AAACCUCACU C AUCAC | 5784 | CUCAU UCAUC CCAUCCACUAUCC CCG ACUGAGCUUU | 15124 |
| 2294 | ACUUUAGACU C UCAUG | 5785 | CAUGA UGAUG CCAUGCACUAUGC CCC ACUCUAAAGU | 15125 |
| 2299 | AGACUGUCAU G CUAAU | 5786 | AUUAG UGAUG GCAUGCACUAUGC GCG AUGACAGUCU | 15126 |
| 2308 | UGCUAAUGGU G UCCCC | 5787 | GGGGA UGAUG GCAUGCACUAUGC GCG ACCAUUAGCA | 15127 |
| 2314 | UGGUGUCCCC G AUCCG | 5788 | AGGCU UGAUG GCAUGCACUAUGC GCG GGGGACACCA | 15128 |
| 2394 | GAAGCAGCAC G CUGUU | 5789 | AACAG UGAUG GCAUGCACUAUGC GCG GUGCUGCUUC | 15129 |
| 2397 | GCAGCACGCU G UUUAU | 579G | AUAAA UGAUG GCAUGCACUAUGC GCG AGCGUGCUGC | 15138 |
| 2404 | GCUGUUUAUU G APAGA | 5791 | UCUUU UGAUG GCAUGCACUAUGC GCG AAUAAACAGC | 15131 |
| 2425 | AGAAGAGGAU G AAGGU | 5792 | ACCUU UGAUG GCAUGCACUAUGC GCG AUCCUCGUCG | 15132 |
| 2431 | GGAUGAAGGU G UCUAU | 5793 | AUAGA UGAUG GCAUGCACUAUGC GCG ACCUUCAUCC | 15133 |
| 2441 | GUCUAUCACU G CAAAG | 5794 | CUUUG UGAUG GCAUGCACUAUGC GCC AGUGAUAGAC | 15134 |
| 2467 | GAAGCGCUCU G UGGAA | 5795 | UUCCA UGAUG GCAUGCACUAUGC GCG AGAGCCCUUC | 15135 |
| 2491 | AUACCUCACU G UUCAA | 5796 | UUGAA UGAUG GCAUGCACUAUGC GCG AGUGAGGUAU | 15136 |
| 2526 | AUCUGGAGCU G AUCAC | 5797 | GUGAU UGAUG GCAUGCACUAUGC GCG AGCUCCAGAU | 15137 |
| 254G | ACUCUAACAU G CACCU | 5798 | AGGUG UGAUG GCAUGCACUAUGC GCG AUGUUAGAGU | 15138 |
| 2546 | ACAUGCACCU G UGUGG | 5799 | CCACA UGAUG GCAUGCACUAUGC GCG AGGUGCAUGU | 15139 |
| 2548 | AUGCACCUGU G UGGCU | 580G | AGCCA UGAUG GCAUGCACUAUGC GCC ACAGGUGCAU | 15140 |
| 2554 | CUGUGUGGCU G CGACU | 5801 | AGUCG UGAUG GCAUGCACUAUGC GCG AGCCACACAG | 15141 |
| 2556 | GUGUGGCUGC G ACUCU | 5802 | AGAGG UGAUG GCAUGCACUAUGC GCG GCAGCCACAC | 15142 |
| 2591 | CUCCUUAUCC G AAAAA | 5803 | UUUUU UGAUG GCAUGCACUAUGC GCC GGAUAAGGAG | 15143 |
| 2598 | UCCGAAAAAU G AAAAG | 5804 | CUUUU UGAUG GCAUGCACUAUGC GCG AUUUUUCGGA | 15144 |
| 2614 | GUCUUCUUCU G AAAUA | 5805 | UAUUU UGAUG GCAUGCACUAUGC GCG AGAAGAAGAC | 15145 |
| 2626 | AAUAAAGACU G ACUAC | 5806 | GUAGU UGAUG GCAUGCACUAUGC GCG AGUCUUUAUU | 15146 |
| 2656 | GGACCCAGAU C AAGUU | 5807 | AACUU UGAUG GCAUGCACUAUCC GCG AUCUGGGUCC | 15147 |
| 2671 | UCCUUUGGAU G ACCAG | 5808 | CUGCU UGAUG GCAUCCACUAUGC GCG AUCCAAAGGA | 15148 |
| 2678 | CAUGACCAGU G UGAGC | 5809 | GCUCA UCAUG GCAUGCACUAUCC GCG ACUGCUCAUC | 15149 |
| 268G | UGAGCAGUGU G ACCGC | 581G | CCGCU UGAUC GCAUGCACUAUGC CCC ACACUGCUCA | 15150 |
| 2695 | GCUCCCUUAU C AUCCC | 5811 | GGCAU UGAUG GCAUGCACUAUGC CCG AUAAGGGAGC | 15151 |
| 2698 | CCCUUAUGAU G CCAGC | 5812 | CCUGG UGAUG GCAUCCACUAUCC GCG AUCAUAAGGG | 15152 |
| 2716 | GUGGGACUUU G CCCGG | 5813 | CCGGG UCAUG CCAUGCACUAUGC GCG AAACUCCCAC | 15153 |
| 2813 | UCACCUACGU G CCGGA | 5814 | UCCGG UGAUC CCAUCCACUAUGC GCC ACCUAGGUCA | 15154 |
| 2821 | GUGCCGGACU G UCCCU | 5815 | AGCCA UGAUG GCAUGCACUAUGC CCG ACUCCGGCAC | 15155 |
| 2827 | GACUGUCGCG G UGAAA | 5816 | UUUCA UGAUG GCAUGCACUAUCC CCC AGCCACACUC | 15156 |
| 2829 | CUCUCCCUGU G AAAAU | 5817 | AUUUU UCAUC CCAUCCACUAUGC CCC ACACCCACAC | 15157 |
| 2835 | CUGUGAAAAU C CUGAA | 5818 | UUCAC UCAUG GCAUGCACUAUCC CCC AUUUUCACAG | 15158 |
| 2838 | UGAAAAUCCU C AAAGA | 5819 | UCUUU UCAUG CCAUCCACUAUCC GCC AGCAUUUUCA | 15159 |
| 286G | CACCCCCAGC G AGUAC | 582G | CUACG UCAUG CCAUCCACUAUGC CCC GCUCGCCGUC | 1516G |
| 2874 | ACAAAGCUCU C AUGAC | 5821 | CUCAU UCAUC CCAUGCACUAUCC CCC AGAGCUUUGU | 15161 |
| 2877 | AAGCUCUGAU C ACUCA | 5822 | UCAGU UCAUG CCAUGCACUAUCC CCC AUCAGAGCUU | 15162 |
| 2881 | UCUCAUCACU C AGCUA | 5823 | UACCU UCAUG CCAUGCACUAUCC CCC ACUCAUCACA | 15163 |
| 2895 | UAAAAAUCUU C ACCCA | 5824 | UCGGG UCAUC CCAUCCACUAUCC CCC AAGAUUUUUA | 15164 |
| 2916 | GCCACCAUCU G AACCU | 5825 | ACGUU UGAUC CCAUCCACUAUCC CCC AGAUCCUCGC | 15165 |
| 2931 | UGGUUAACCU C CUCGC | 5826 | CCCAC UCAUG GCAUGCACUAUCC CCC ACGUUAACCA | 15166 |
| 2942 | CUCCCAGCCU C CACCA | 5827 | UCCUC UGAUC CCAUCCACUAUCC CCC ACCCUCCCAG | 15167 |
| 2964 | CAGCGCCUCU C AUCCU | 5828 | ACCAU UCAUC CCAUGCACUAUGC CCC ACACCCCCUC | 15168 |
| 297G | CUCUGAUCCU C AUUGU | 5829 | ACAAU UCAUC CCAUGCACUAUCC CCC ACCAUCACAC | 15169 |
| 2974 | CAUGGUGAUU C UUGAA | 583G | UUCAA UGAUC CCAUCCACUAUCC CCC AAUCACCAUC | 15170 |
| 2977 | GGUGAUUGUU C AAUAC | 5831 | GUAUU UCAUC CCAUCCACUAUCC CCC AACAAUCACC | 15171 |
| 2984 | CUUCAAUACU C CAAAU | 5832 | AUUUG UCAUC CCAUGCACUAUCC CCC ACUAUUCAAC | 15172 |
| 3025 | CACCAAACGU G ACUUA | 5833 | UAACU UCAUG CCAUGCACUAUCC CCC ACGUUUCCUC | 15173 |
| 3049 | CAACAAGGAU C CACCA | 5834 | UCCUC UCAUG CCAUGCACUAUCC CCC AUCCUUGUUG | 15174 |
| 3139 | CACCAGCAGC C AAAGC | 5835 | CCUUU UCAUG GCAUCCACUAUGC GCC GCUGCUGGUG | 15175 |
| 3148 | CGAAAGCUUU C CGAGC | 5836 | GCUCG UGAUC CCAUCCACUAUCC GCC AAACCUUUCC | 15176 |
| 3150 | AAACCUUUGC G AGCUC | 5837 | GAGCU UGAUC CCAUGCACUAUCC GCC CCAAACCUUU | 15177 |
| 3180 | AUAAAAGUCU C AGUGA | 5838 | UCACU UGAUG GCAUGCACUAUGC GCG AGACUUUUAU | 15178 |
| 3184 | AAGUCUGAGU G AUGUU | 5839 | AACAU UGAUC CCAUCCACUAUCC CCC ACUCAGACUU | 15179 |
| 3187 | UCUGAGUGAU G UUGAG | 5840 | CUCAA UGAUG GCAUGCACUAUCC GCG AUCACUCAGA | 15180 |
| 3190 | GACUCAUCUU G AGCAA | 5841 | UUCCU UGAUC CCAUGCACUAUCC CCG AACAUCACUC | 15181 |
| 3208 | GCAGCAUUCU G ACCGU | 5842 | ACCGU UGAUC CCAUCCACUAUGC GCC AGAAUCCUCC | 15182 |
| 3246 | UGCAAGAUCU G AUCUC | 5843 | GAAAU UCAUC GCAUGCACUAUGC GCC ACAUCUUCCA | 15183 |
| 3288 | UGGACUUCCU C UCUUC | 5844 | GAAGA UGAUG CCAUGCACUAUCC CCG ACGAACUCCA | 15184 |
| 3302 | UCCAGAAAGU G CAUUC | 5845 | GAAUG UGAUC GCAUGCACUAUCC GCG ACUUUCUCGA | 15185 |
| 3324 | ACCUCGCACC G AGAAA | 5846 | UUUCU UCAUC GCAUGCACUAUGC GCC GCUGCCAGCU | 15186 |
| 3343 | UCUUUUAUCU G AGAAC | 5847 | CUUCU UCAUC GCAUCCACUAUGC GCC AGAUAAAAGA | 15187 |
| 3357 | ACAACCUCGU G AAGAU | 5848 | AUCUU UGAUG CCAUCCACUAUGC CCC ACCACGUUGU | 15188 |
| 3365 | GUCAAGAUUU C UGAUU | 5849 | AAUCA UGAUG GCAUCCACUAUCC GCG AAAUCUUCAC | 15189 |
| 3367 | GAAGAUUUCU G AUUUU | 5850 | AAAAU UCAUG GCAUCCACUAUGC CCC ACAAAUCUUC | 15190 |
| 3379 | UUUUCCCCUU G CCCGG | 5851 | CCGGC UCAUG CCAUCCACUAUGC GCC AAGCCCAAAA | 15191 |
| 3403 | UAACAACCCC C AUUAU | 5852 | AUAAU UCAUC CCAUCCACUAUGC CCC CGCCUUCUUA | 15192 |
| 3409 | CCCCCAUUAU C UCACA | 5853 | UCUCA UCAUC CCAUCCACUAUCC CCC AUAAUCCCCC | 15193 |
| 3411 | CCCAUUAUCU C ACAAA | 5854 | UUUCU UCAUC CCAUGCACUAUCC CCC ACAUAAUCCC | 15194 |
| 3428 | CCACAUACUC C ACUUC | 5855 | CAACU UCAUC GCAUCCACUAUCC CCC CACUAUCUCC | 15195 |
| 3438 | CACUUCCUCU C AAAUC | 5856 | CAUUU UCAUC CCAUCCACUAUCC CCC ACACGAACUC | 15196 |
| 3454 | CAUGCCUCCC C AAUCU | 5857 | ACAUU UCAUC CCAUCCACUAUCC CCC CCCACCCAUC | 15197 |
| 3466 | AUCUAUCUUU C ACAAA | 5858 | UUUCU UCAUC CCAUCCACUAUCC CCC AAACAUACAU | 15198 |
| 3490 | CACCAACACC C ACCUC | 5859 | CACCU UCAUC CCAUCCACUAUCC CCC CCUCUUCCUC | 15199 |
| 3495 | ACACCGACCU C UCCUC | 586G | CACCA UCAUC CCAUCCACUAUCC CCC ACCUCCCUCU | 15200 |
| 3513 | ACCCACUAUU C CUCUC | 5861 | CACAC UCAUC CCAUCCACUAUCC CCC AAUACUCCCU | 15201 |
| 3516 | CACUAUUCCU C UCCCA | 5862 | UCCCA UCAUC CCAUCCACUAUCC CCC ACCAAUACUC | 15202 |
| 3568 | ACAAAUCCAU C ACCAC | 5863 | CUCCU UCAUC CCAUCCACUAUCC CCC AUCCAUUUCU | 15203 |
| 3578 | CACCACUUUU C CACUC | 5864 | CACUC UCAUC CCAUCCACUAUCC CCC AAAAGUCCUC | 15204 |
| 3584 | UUUUCCACUC C CCUCA | 5865 | UCACC UCAUC CCAUCCACUAUCC CCC CACUCCAAAA | 15205 |
| 3588 | CCACUCCCCU C ACCCA | 5866 | UCCCU UCAUC CCAUCCACUAUCC CCC ACCCCACUCC | 15206 |
| 3600 | CCCAACCCAU C ACCAU | 5867 | AUCCU UCAUC CCAUCCACUAUCC CCC AUCCCUUCCC | 15207 |
| 3606 | CCAUCAGGAU C ACACC | 5868 | CCUCU UCAUC CCAUCCACUAUCC CCC AUCCUCAUCC | 15208 |
| 3616 | CACACCUCCU C ACUAC | 5869 | GUACU UCAUC CCAUCCACUAUCC CCC ACCACCUCUC | 15209 |
| 3631 | CUCUACUCCU C AAAUC | 5870 | CAUUU UCAUC CCAUCCACUAUCC CCC ACCACUACAC | 1521G |
| 3648 | AUCACAUCAU C CUCCA | 5871 | UCCAC UCAUC CCAUCCACUAUCC CCC AUCAUCUCAU | 15211 |
| 3656 | AUCCUCCACU C CUCCC | 5872 | GCCAC UCAUC CCAUCCACUAUCC CCC ACUCCACCAU | 15212 |
| 3691 | CCCAACAUUU C CACAA | 5873 | UUCUC UCAUC CCAUCCACUAUCC CCC AAAUCUUCCC | 15213 |
| 3700 | UCCACAACUU C UCCAA | 5874 | UUCCA UCAUC CCAUCCACUAUCC CCC AACUUCUCCA | 15214 |
| 3715 | AAAACUACGU C AUUUC | 5875 | CAAAU UCAUC CCAUCCACUAUCC CCC ACCUACUUUU | 15215 |
| 372G | UACCUCAUUU C CUUCA | 5876 | UCAAC UCAUC CCAUCCACUAUCC CCC AAAUCACCUA | 15216 |
| 3733 | UCAACCAAAU C UACAA | 5877 | UUCUA UCAUC CCAUCCACUAUGC CCC AUCUCCUUCA | 15217 |
| 3769 | CCCAAUCAAU C CCAUA | 5878 | UAUCC UCAUC CCAUCCACUAUCC CCC AUUCAUUCCC | 15218 |
| 3777 | AUCCCAUACU C ACACC | 5879 | CCUCU UGAUC CCAUCCACUAUCC CCC ACUAUCCCAU | 15219 |
| 3811 | CUCAACUCCU C CCUUC | 5880 | CAACC UCAUC CCAUCCACUAUGC CCC ACCACUUCAC | 1522G |
| 382G | UCCCUUCUCU C ACCAC | 5881 | CUCCU UCAUC CCAUCCACUAUCC CCC ACACAACCCA | 15221 |
| 3852 | UUUCACCUCC C AACUU | 5882 | AACUU UCAUC CCAUCCACUAUCC CCC CCACCUCAAA | 15222 |
| 3874 | AGCAACCUCU C AUCAU | 5883 | AUCAU UCAUC GCAUGCACUAUGC CCC ACACCUUCCU | 15223 |
| 3877 | AAGCUCUGAU C AUGUC | 5884 | CACAU UGAUG GCAUGCACUAUGC CCC AUCAGAGCUU | 15224 |
| 3880 | CUCUGAUGAU C UCACA | 5885 | UCUGA UGAUG GCAUGCACUAUGC GCG AUCAUCAGAG | 15225 |
| 3889 | UGUCAGAUAU G UAAAU | 5886 | AGUCA UCAUG GCAUCCACUAUCC GCG AUAUCUGACA | 15226 |
| 3895 | AUAUGUAAAU C CUUUC | 5887 | GAAAG UGAUG CCAUCCACUAUGC GCG AUUUACAUAU | 15227 |
| 3909 | UCAACUUCAU C AGCCU | 5888 | ACGCU UCAUG CCAUCCACUAUGC CCC AUCAACUUCA | 15228 |
| 3934 | CAAAACCUUU G AAGAA | 5889 | UUCUU UCAUC CCAUGCACUAUCC GCC AAAGCUUUUC | 15229 |
| 3948 | AACUUUUACC G AAUCC | 5890 | GCAUU UGAUC GCAUCCACUAUGC CCC CGUAAAAGUU | 15230 |
| 3952 | UUUACCGAAU G CCACC | 5891 | GGUGG UGAUG CCAUGCACUAUGC GCG AUUCGCUAAA | 15231 |
| 3963 | CCACCUCCAU C UCUGA | 5892 | UCAAA UGAUG CCAUGCACUAUGC CCC AUGGAGGUCC | 15232 |
| 3967 | CUCCAUGUUU C AUGAC | 5893 | CUCAU UCAUC GCAUGCACUAUGC CCC AAACAUCGAG | 15233 |
| 3970 | CAUGUUUCAU C ACUAC | 5894 | GUAGU UGAUG CCAUCCACUAUCC CCG AUCAAACAUG | 15234 |
| 3982 | CUACCAGCCC C ACACC | 5895 | CCUGU UGAUG CCAUCCACUAUCC CCC CCCCUGGUAG | 15235 |
| 3996 | GCAGCACUCU C UUCCC | 5896 | GCCAA UCAUG CCAUCCACUAUCC CCC AGAGUCCUCC | 15236 |
| 4011 | CCUCUCCCAU C CUCAA | 5897 | UUCAG UCAUC GCAUGCACUAUGC GCG AUGGGAGAGG | 15237 |
| 4014 | CUCCCAUGCU C AAGCG | 5898 | CCCUU UGAUC GCAUGCACUAUCC CCC AGCAUGCGAG | 15238 |
| 4019 | AUCCUCAACC C CUUCA | 5899 | UCAAC UCAUC CCAUCCACUAUCC CCC CCUUCACCAU | 15239 |
| 4033 | CACCUCCACU C ACACC | 5900 | CCUGU UCAUC CCAUCCACUAUCC CCC AGUCCAGGUC | 15240 |
| 4053 | CCAACCCCUC C CUCAA | 5901 | GUCAC UCAUC CCAUCCACUAUCC CCC CACCCCUUCC | 15241 |
| 4063 | CCUCAACAUU C ACUUC | 5902 | CAACU UCAUC CCAUCCACUAUCC CCC AAUCUUCACC | 15242 |
| 4068 | ACAUUCACUU C ACACU | 5903 | ACUCU UCAUC GCAUCCACUAUCC CCC AACUCAAUCU | 15243 |
| 4101 | ACUCCCCCCU C UCUCA | 5904 | UCACA UCAUC CCAUCCACUAUCC CCC ACCCCCCACU | 15244 |
| 4105 | CCGCCUCUCU C AUCUC | 5905 | CACAC UCAUC CCAUCCACUAUCC CCC ACACACCCCC | 15245 |
| 4108 | CCUCUCUCAU C UCACC | 5906 | CCUGA UCAUC CCAUCCACUAUCC CCC AUCACACACC | 15246 |
| 4127 | CCCAGUUUCU C CCAUU | 5907 | AAUCC UCAUG CCAUCCACUAUCC CCC ACAAACUCCC | 15247 |
| 4139 | CAUUCCACCU C UCCCC | 5908 | GCCCA UCAUC CCAUCCACUAUCC CCC ACCUCCAAUG | 15248 |
| 4153 | CCACCUCACC C AAGGC | 5909 | CCCUU UCAUC CCAUCCACUAUGC CCC CCUCACCUCC | 15249 |
| 4163 | CAAGGCAACC C CACCU | 591G | ACCUC UCAUC GCAUCCACUAUCC CCC CCUUCCCUUC | 15250 |
| 4177 | CUUCACCUAC C ACCAC | 5911 | CUCCU UGAUC CCAUCCACUAUCC CCC CUACCUCAAC | 15251 |
| 4183 | CUACCACCAC C CUCAC | 5912 | CUCAC UCAUC CCAUCCACUAUCC CCC GUCGUCCUAC | 15252 |
| 4186 | CCACCACCCU C ACCUC | 5913 | CACCU UCAUC CCAUGCACUAUCC CCC ACCCUCCUCC | 15253 |
| 4204 | AACCAAAAUC C CCUCC | 5914 | CCACC UGAUC CCAUCCACUAUCC CCC CAUUUUCCUU | 15254 |
| 4208 | AAAAUCCCCU C CUCCU | 5915 | ACCAC UCAUC CCAUCCACUAUCC CCC ACCCCAUUUU | 15255 |
| 4211 | AUCCCCUCCU C CUCCC | 5916 | CCCAC UCAUC CCAUCCACUAUCC CCC ACCACGCCAU | 15256 |
| 4218 | CCUCCUCCCC C CCCCC | 5917 | CCCCC UCAUC CCAUCCACUAUCC CCC CCCCACCACC | 15257 |
| 4245 | CCCUCCUCCU C GACUC | 5918 | CACUA UCAUC CCAUCCACUAUCC CCC ACCACCACCC | 15258 |
| 4272 | UCUACAGGUG C ACACC | 5919 | CCUCU UCAUC CCAUCCACUAUCC CCC AAACUCUACA | 15259 |
| 4277 | ACUUUCACAC C AACCC | 592G | CCCUU UCAUC CCAUCCACUAUCC CCG CUCUCAAACU | 15260 |
| 4301 | ACAACCACAU C UCUAG | 5921 | AGACA UCAUC CCAUCCACUAUCC CCC AUCUCCUUCU | 15261 |
| 4303 | AACCACAUCU C UAUUU | 5922 | AAAUA UCAUC CCAUCCACUAUCC CCC ACAUCUCCUU | 15262 |
| 4332 | ACUACCUUUU C CCACU | 5923 | ACUCC UCAUC CCAUCCACUAUCC CCC AAAACCUACU | 15263 |
| 4343 | CCACUAUUAU C CAGAG | 5924 | AGAUC UCAUC CCAUCCACUAUCC CCC AUAAUACUCC | 15264 |
| 4386 | GCACCCACCU C CUUUU | 5925 | AAAAC UCAUC CCAUCCACUAUCC CCC ACCUCCCUCC | 15265 |
| 4393 | CCUCCUUUUU C UCAUU | 5926 | AAUCA UCAUC CCAUCCACUAUCC CCC AAAAACCACC | 15266 |
| 4395 | UCCUUUUUCU C AUUUU | 5927 | AAAAU UCAUC CCAUCCACUAUCC CCC ACAAAAACCA | 15267 |
| 441G | UUUUAAUACU C CLUUU | 5928 | AAAAC UCAUC CCAUCCACUAUCC CCC ACUAUUAAAA | 15268 |
| 4423 | UUUUUUUUUU C ACUAA | 5929 | UUACU UCAUC CCAUCCACUAUCC CCC AAAAAAAAAA | 15269 |
| 4436 | UAACAACAAU C UAACU | 5930 | ACUUA UCAUC CCAUCCACUAUCC CCC AUUCUUCUUA | 15270 |
| 4459 | CACAAAUACU C ACAAC | 5931 | CUUCU UCAUC CCAUCCACUAUCC CCC ACUAUUUCUC | 15271 |
| 4466 | ACUCACAACU C AACAA | 5932 | UUCUU UCAUC CCAUCCACUAUCC CCC ACUUCUCACU | 15272 |
| 4479 | GAACACUACU G CUAAA | 5933 | UUUAG UGAUG GCAUGCACUAUGC GCG AGUAGUGUUC | 15273 |
| 4492 | AAAUCCUCAU G UUACU | 5934 | AGUAA UGAUG GCAUGCACUAUGC GCG AUGAGGAUUU | 15274 |
| 4502 | GUUACUCAGU G UUAGA | 5935 | UCUAA UGAUG GCAUGCACUAUGC GCG ACUGAGUAAC | 15275 |
| 4529 | UAAACCCAAU G ACUUC | 5936 | GAAGU UGAUG GCAUGCACUAUGC GCG AUUGGGUUUA | 15276 |
| 4538 | UGACUUCCCU C CUCCA | 5937 | UGGAG UGAUG GCAUGCACUAUGC GCG AGGGAAGUCA | 15277 |
| 4550 | UCCAACCCCC C CCACC | 5938 | GGUGG UGAUG GCAUGCACUAUGC GCG GGGGGUUGGA | 15278 |
| 4565 | CUCAGGGCAC C CAGGA | 5939 | UCCUG UGAUG GCAUGCACUAUGC GCG GUGGCCUGAG | 15279 |
| 4578 | GGACCAGUUU G AUUGA | 5940 | UCAAU UGAUG GCAUGCACUAUGC GCG AAACUCGUCC | 15280 |
| 4582 | CAGUUUGAUU G AGGAG | 5941 | CUCCU UGAUG GCAUGCACUAUGC GCG AAUCAAACUG | 15281 |
| 4590 | UUGAGGAGCU C CACUG | 5942 | CAGUC UGAUG GCAUGCACUAUGC GCC AGCUCCUCAA | 15282 |
| 4595 | GAGCUGCACU G AUCAC | 5943 | GUGAU UGAUG GCAUGCACUAUGC GCG AGUGCAGCUC | 15283 |
| 4606 | AUCACCCAAU G CAUCA | 5944 | UGAUG UGAUG GCAUGCACUAUGC GCG AUUGGGUGAU | 15284 |
| 4634 | GGCCAGCCCU G CAGCC | 5945 | GGCUG UGAUG GCAUGCACUAUGC GCG AGGGCUGGCC | 15285 |
| 4691 | UGGCUGGCCU G AGCAA | 5946 | UUGCU UGAUG CCAUGCACUAUGC GCG AGGCCAGCCA | 15286 |
| 4730 | CCUAAGACAU G UGAGG | 5947 | CCUCA UGAUG GCAUGCACUAUGC GCG AUCUCUUAGG | 15287 |
| 4732 | UAAGACAUGU G AGGAG | 5948 | CUCCU UGAUG GCAUGCACUAUGC CGG ACAUGUCUUA | 15288 |
| 4792 | GAGAAGGCAU G AGAAA | 5949 | UUUCU UGAUG GCAUGCACUAUGC GCG AUGCCUUCUC | 15289 |
| 4804 | GAAAGAAUUU G AGACG | 5950 | CGUCU UGAUG GCAUGCACUAUGC GCG AAAUUCUUUC | 15290 |
| 4809 | AAUUUGAGAC G CACCA | 5951 | UGGUG UGAUG GCAUGCACUAUCC GCG GUCUCAAAUU | 15291 |
| 4816 | GACGCACCAU G UGGGC | 5952 | GGCCA UGAUG GCAUGCACUAUGC GCG AUGGUGCGUC | 15292 |
| 4847 | GCUCAGCAAU G CCAUU | 5953 | AAUGG UGAUG GCAUGCACUAUGC GCG AUUGCUGAGC | 15293 |
| 4872 | UCCCAGCUCU G ACCCU | 5954 | AGGGU UGAUG GCAUGCACUAUGC GCG AGAGCUGGGA | 15294 |
| 4887 | UUCUACAUUU C ACGGC | 5955 | GCCCU UGAUG CCAUGCACUAUGC CCC AAAUGUAGAA | 15295 |
| 4916 | GAUCCACAGC C AUCAG | 5956 | CUCAU UCAUG CCAUGCACUAUGC CGG CGUGUCCAUC | 15296 |
| 4919 | CCACACCCAU C ACCCG | 5957 | CCCCU UCAUC GCAUGCACUAUCC CCC AUCCGUCUCG | 15297 |
| 5018 | GUGGUUCUAU C UCCAU | 5958 | AUCCA UGAUC CCAUGCACUAUCC CCC AUAGAACCAC | 15298 |
| 5039 | UUCGUGGCAU C UUUUG | 5959 | CAAAA UCAUG GCAUCCACUAUCC CCC AUGCCACGAA | 15299 |
| 5044 | GGCAUGUUUU C AUUUC | 5960 | CAAAU UGAUG GCAUGCACUAUCC CCC AAAACAUGCC | 15300 |
| 5049 | GUUUUGAUUU C UACCA | 5961 | UGCUA UGAUC CCAUCCACUAUGC CCC AAAUCAAAAC | 15301 |
| 5057 | UUCUAGCACU C AGGGU | 5962 | ACCCU UCAUC CCAUGCACUAUGC CCC AGUCCUACAA | 15302 |
| 5076 | ACUCAACUCU C ACCGC | 5963 | CCCCU UCAUC CCAUCCACUAUCC CCC ACAGUUCAGU | 15303 |
| 5107 | CUAGUAACAU C CACUC | 5964 | CACUC UCAUC CCAUGCACUAUCC CGG AUCUUACUAC | 15304 |
| 5112 | AACAUCCACU C AAAAC | 5965 | CUUUU UCAUC CCAUCCACUAUCC CCC ACUGCAUCUU | 15305 |
| 5135 | GAGUUAGGUU C UCUCC | 5966 | GCACA UGAUC CCAUCCACUAUCC CCC AACCUAACUC | 15306 |
| 5148 | UCCACGCCAU C AUCCG | 5967 | GCCAU UCAUC CCAUCCACUAUCC CCC AUCCGCUCCA | 15307 |
| 5162 | CCCUUACACU C AAAAU | 5968 | AUUUU UCAUC CCAUCCACUAUGC CCC AGUGUAAGCC | 15308 |
| 5168 | CACUGAAAAU C UCACA | 5969 | UCUCA UCAUG CCAUGCACUAUCC CCC AUUUUCACUC | 15309 |
| 5234 | GUAUUAUUCU C UUUUG | 5970 | CAAAA UGAUC CCAUGCACUAUCC CCC ACAAUAAUAC | 15310 |
| 5239 | AUUCUCUUUU C CACAC | 5971 | CUGUC UCAUC CCAUCCACUAUCC CCC AAAACAGAAU | 15311 |
| 5251 | ACAGUUACUU C UGAAA | 5972 | UUUCA UCAUC CCAUCCACUAUCC CCC AACUAACUGU | 15312 |
| 5253 | AGUUAGUUGU C AAAGA | 5973 | UCUUU UCAUC CCAUCCACUAUGC CCC ACAACUAACU | 15313 |
| 5264 | AAACAAACCU C ACAAG | 5974 | CUUCU UCAUC CCAUCCACUAUCC CCC ACCUUUCUUU | 15314 |
| 5273 | UCACAAGAAU C AAAAU | 5975 | AUUUU UCAUC CCAUCCACUAUCC CCC AUUCUUCUCA | 15315 |
| 5279 | GAAUCAAAAU C CACUC | 5976 | CACUC UCAUC CCAUCCACUAUCC CCC AUUUUCAUUC | 15316 |
| 5287 | AUCCACUCCU C ACCAC | 5977 | CUCCU UGAUC GCAUGCACUAUCC CCC ACCACUCCAC | 15317 |
| 5313 | AUAUCAAAAC C ACCCC | 5978 | CCCCU UGAUC CCAUCCACUAUCC CCC CUUUUCAUAU | 15318 |
| 532G | AACGAGGCCU C AUCCA | 5979 | UCCAU UCAUC CCAUCCACUAUCC CCC ACCCCUCCUU | 15319 |
| 5469 | ACACUAAUCU C AAACC | 5980 | CCUUU UCAUC CCAUCCACUAUCC CCC ACAUUACUCU | 15320 |
| 5477 | CUCAAACCAU C UCCAA | 5981 | UUCCA UCAUC GCAUCCACUAUCC CCC AUCCUUUCAC | 15321 |
| 5497 | AUUAGCUGGC G CAUAU | 5982 | AUAUG UGAUG GCAUGCACUAUGC GCG GCCAGCUAAU | 15322 |
| 5522 | UAAGCUCCUU C AGUAA | 5983 | UUACU UGAUG GCAUCCACUAUGC CCC AAGGACCUUA | 15323 |
| 5539 | AAGGUGGUAU G UAAUU | 5984 | AAUUA UGAUC GCAUGCACUAUGC CCG AUACCACCUU | 15324 |
| 5548 | UCUAAUUUAU C CAAGG | 5985 | CCUUG UCAUC CCAUGCACUAUCC GCC AUAAAUUACA | 15325 |
| 5589 | AUUAGUUAAU C AGCCA | 5986 | UGCCU UCAUG CCAUCCACUAUCC CCC AUUAACUAAU | 15326 |
| 5623 | AUUUUCAACU G CUUUC | 5987 | CAAAG UGAUG GCAUGCACUAUGC GCG AGUUGAAAAU | 15327 |
| 5628 | CAACUGCUUU G AAACU | 5988 | ACUUU UGAUC CCAUGCACUAUGC GCG AAACCAGUUG | 15328 |
| 5635 | UUUGAAACUU G CCUCG | 5989 | CCACC UCAUC GCAUGCACUAUGC GCC AAGUUUCAAA | 15329 |
| 5646 | CCUCGGCUCU G ACCAC | 5990 | AUGCU UGAUG GCAUCCACUAUCC CCG ACACCCCACC | 15330 |
| 5652 | GUCUCAGCAU C AUCCC | 5991 | CCCAU UGAUC GCAUCCACUAUCC GCG AUCCUCAGAC | 15331 |
| 5684 | ACGAAAGCGC G CCUAC | 5992 | GUAGC UCAUC GCAUGCACUAUGC CCG GCCCUUUCCU | 15332 |
| 5725 | GCCUUCCAUC G CUAAC | 5993 | CUGAG UCAUG CCAUCCACUAUGC CCG GAUCCAAGGC | 15333 |
| 5739 | ACCUGGCUCU G UUUGA | 5994 | UCAAA UCAUG CCAUCCACUAUCC GCC AGACCCACCU | 15334 |
| 5743 | GGCUCUCUUU C AUCCU | 5995 | AGCAU UCAUG GCAUGCACUAUCC GCG AAACACAGCC | 15335 |
| 5746 | UCUGUUUCAU G CUAUU | 5996 | AAUAC UGAUG CCAUGCACUAUCC GCC AUCAAACAGA | 15336 |
| 5755 | UGCUAUUUAU C CAAGU | 5997 | ACUUC UCAUC GCAUGCACUAUGC GCC AUAAAUACCA | 15337 |
| 5771 | UAGGGUCUAU G UAUUU | 5998 | AAAUA UCAUG CCAUGCACUAUCC GCC AUACACCCUA | 15338 |
| 5782 | UAUUUACCAU C CCCCU | 5999 | ACCCC UCAUC CCAUCCACUAUCC CCC AUCCUAAAUA | 15339 |
| 5784 | UUUACCAUCC C CCUAC | 6000 | CUACC UCAUC CCAUCCACUAUCC CCC CCAUCCUAAA | 15340 |
| 5825 | CCCUUCCAUC C CUAAC | 5993 | CUUAC UCAUC CCAUGCACUAUCC CCC CAUCCAACCC | 15333 |
| 5839 | ACCUCCCUCU C UUUCA | 5994 | UCAAA UCAUC CCAUCCACUAUCC CCG ACACCCACCU | 15334 |
| 5843 | CCCUCUCUUU C AUCCU | 5995 | ACCAU UGAUC CCAUCCACUAUCC CCC AAACACACCC | 15335 |
| 5846 | UCUCUUUCAU C CUAUU | 5996 | AAUAC UCAUC CCAUCCACUAUCC CCC AUCAAACACA | 15336 |
| 5855 | UCCUAUUUAU C CAACU | 5997 | ACUUC UCAUC CCAUCCACUAUCC CCC AUAAAUAGCA | 15337 |
| 5871 | UACCCUCUAU C UAUUU | 5998 | AAAUA UCAUC CCAUCCACUAUCC CCC AUACACCCUA | 15338 |
| 5882 | UAUUUACCAU C UCUCC | 6001 | CCACA UCAUC CCAUCCACUAUCC CCC AUCCUAAAUA | 15341 |
| 5886 | UACCAUCUCU C CACCU | 6002 | ACCUC UCAUC CCAUCCACUAUCC CCC ACACAUCCUA | 15342 |
| 5895 | UCCACCUUCU C CACCC | 6003 | CCCUC UCAUC CCAUCCACUAUCC CCC ACAACCUCCA | 15343 |
| 5931 | ACACUCCAUU C CUCCU | 6004 | ACCAC UCAUC CCAUCCACUAUCC CCC AAUCCACUCU | 15344 |
| 5934 | CUCCAUUCCU C CUUCU | 6005 | ACAAC UCAUC CCAUCCACUAUCC CCC ACCAAUCCAC | 15345 |
| 5955 | ACAACACUAU C CUUCC | 6006 | CCAAC UCAUC CCAUCCACUAUCC CCC AUACUCUUCU | 15346 |
| 5971 | UUUUAUCCAU C UAAUU | 6007 | AAUUA UCAUC CCAUCCACUAUCC CCC AUCCAUAAAA | 15347 |
| 5982 | UAAUUUAACU C UACAA | 6008 | UUCUA UCAUC CCAUCCACUAUCC CCC ACUUAAAUUA | 15348 |
| 5991 | UCUACAACCU C ACCUC | 6009 | CACCU UCAUC CCAUCCACUAUCC CCC ACCUUCUACA | 15349 |
| 6006 | CUAACUAACC C AACAA | 601G | UUCUU UCAUC CCAUCCACUAUCC CCC CCUUACUUAC | 15350 |
| 6013 | ACCCAACAAU C UAUCC | 6011 | CCAUA UCAUC CCAUCCACUAUCC CCC AUUCUUCCCU | 15351 |
| 6017 | AACAAUCUAU C CCUCU | 6012 | ACACC UCAUC CCAUCCACUAUCC CCC AUACAUUCUU | 15352 |
| 6023 | CUAUCCCUCU C UUCUU | 6013 | AACAA UCAUC CCAUCCACUAUCC CCC ACACCCAUAC | 15353 |
| 6031 | CUCUUCUUAU C UCCCA | 6014 | UCCCA UCAUC CCAUCCACUAUCC CCC AUAACAACAC | 15354 |
| 6033 | CUUCUUAUCU C CCACA | 6015 | UCUCC UCAUC CCAUCCACUAUCC CCC ACAUAACAAC | 15355 |
| 6044 | CCACAUCCUU C UUUAA | 6016 | UUAAA UCAUC CCAUCCACUAUCC CCC AACCAUCUCC | 15356 |
| 6059 | AACCCUCUCU C CAUCA | 6017 | UCAUA UCAUC CCAUCCACUAUCC CCC ACACACCCUU | 15357 |
| 6063 | CUCUCUCUAU C AACAC | 6018 | CUCUU UCAUC CCAUCCACUAUCC CCC AUACACACAC | 15358 |
| 6098 | AUUCCCUACU C ACCCU | 6019 | ACCCU UCAUC CCAUCCACUAUCC CCC ACUAGCCAAU | 15359 |
| 6127 | ACCCCCUUUU C UCCAA | 602G | CUCCA UCAUC CCAUCCACUAUCC CCC AAAACCCCCU | 15360 |
| 6230 | ACAUAUCUUU C UUCUU | 6021 | AACAA UCAUC CCAUCCACUAUCC CCC AAACAUUUCU | 15361 |
| 6233 | AAUCUUUCUU C UUCCU | 6022 | ACCAA UCAUC CCAUCCACUAUCC CCC AACAAACAUU | 15362 |
| 6254 | UUACACAUAC C CAAAC | 6023 | CUUUC UCAUC CCAUCCACUAUCC CCC CUAUCUCUAA | 15363 |
| 6265 | CAAACCACCU C UCACA | 6024 | UCUCA UCAUC CCAUCCACUAUCC CCC ACCUCCUUUC | 15364 |
| 6267 | AACCACCUGU C ACAGC | 6025 | GCUGU UGAUG GCAUGCACUAUGC GCG ACAGGUGGUU | 15365 |
| 6391 | AGCAAAUAGU G AUAAC | 6026 | GUUAU UGAUG GCAUGCACUAUGC CCC ACUAUUUCCU | 15366 |
| 6413 | AACCUUACCU G UUCAU | 6027 | AUCAA UCAUC GCAUGCACUAUCC CCG ACCUAAGCUU | 15367 |
| 6419 | ACCUCUUCAU C UCUUC | 6028 | CAAGA UCAUC CCAUCCACUAUCC UCG AUCAACAGCU | 15368 |
| 6424 | UUCAUGUCUU C AUCUC | 6029 | GAAAU UCAUC GCAUCCACUAUCC CCC AAGACAUGAA | 15369 |
| 6504 | CAUUACAAUU G UUACU | 6030 | AGUAA UCAUG GCAUCCACUAUCC CCG AAUUCUAAUG | 15370 |
| 6532 | ACUCACGUUU G UAGCA | 6031 | UGCUA UGAUG CCAUGCACUAUCC CCC AAACCUCACU | 15371 |
| 6543 | UAGCAUACAU G AGUCC | 6032 | GGACU UCAUG GCAUGCACUAUCC CCC AUCUAUCCUA | 15372 |
| 6588 | CAGUCUUAAU G UAGAA | 6033 | UUCCA UGAUC CCAUGCACUAUCC GCC AUUAAGACUC | 15373 |
| 661G | AUGGACACUC C UAAUA | 6034 | UAUUA UGAUG GCAUGCACUAUGC CCG AAGUCUCCAU | 15374 |
| 6618 | UUGUAAUAAU C ACCUA | 6035 | UACCU UGAUG CCAUGCACUAUCC CCC AUUAUUACAA | 15375 |
| 6634 | GUUACAAAGU G CUUGU | 6036 | ACAAC UGAUG CCAUCCACUAUCC CCC ACUUUGUAAC | 15376 |
| 6638 | CAAAGUGCUU G UUCAU | 6037 | AUGAA UCAUG GCAUGCACUAUGC CCC AACCACUUUC | 15377 |
| 6656 | AAAUACCACU G AAAAU | 6038 | AUCUC UGAUG CCAUGCACUAUCC CCG ACUCCUAUUU | 15378 |
| 6663 | ACUCAAAAUU C AAACA | 6039 | UCUUU UCAUG CCAUCCACUAUCC CCC AAUUUUCACU | 15379 |
| 6670 | AUUCAAACAU C AAUUA | 6040 | UAAUU UGAUG CCAUCCACUAUGC CCC AUGUUUCAAU | 15380 |
| 6679 | UGAAUUAACU C AUAAU | 6041 | AUUAU UGAUG CCAUCCACUAUGC CCC AGUUAAUUCA | 15381 |
| 6698 | CCAAUCAUUU C CCAUU | 6042 | AAUGC UGAUG CCAUCCACUAUCC CCG AAAUCAUUCC | 15382 |
| 6707 | UCCCAUUUAU C ACAAA | 6043 | UUUCU UCAUC CCAUGCACUAUGC CCC AUAAAUCCCA | 15383 |
| 6736 | AACAAAGAAC C AGCAC | 6044 | CUCCU UCAUC CCAUCCACUAUCC CCC CUUCUUUCUU | 15384 |
| 6759 | CACAGUUUCU C ACAUA | 6045 | UAUCU UCAUG CCAUCCACUAUCC CCG ACAAACUCUG | 15385 |
| 6767 | CUCACAUAAU C UACCU | 6046 | ACCUA UCAUC CCAUCCACUAUCC CCC AUUAUCUCAC | 15386 |
| 6798 | CAAUCCCCCU C AAACC | 6047 | CCUUU UCAUC CCAUCCACUAUGC CCC ACCCCCAUUC | 15387 |
| 6806 | CUCAAACCAU C UCCAA | 6048 | CUCCA UCAUC CCAUCCACUAUCC CCC AUCGUUUCAC | 15388 |
| 6808 | CAAACCAUCU C CAACU | 6049 | ACUUC UCAUC CCAUCCACUAUCC CCC ACAUCCUCUC | 15389 |
| 6816 | CUCCAACUCU C UCUCU | 6050 | ACACA UCAUC CCAUGCACUAUCC CCC ACACCUCCAC | 15390 |
| 6818 | CCAACUCUCU C UCUUC | 6051 | CAACA UCAUC CCAUCCACUAUCC CCC ACACACUUCC | 15391 |
| 6823 | UCUCUCUCUU C UCACU | 6052 | ACUCA UCAUC GCAUCCACUAUCC CCC AACACACACA | 15392 |
| 6838 | UCCAACAACU C ACACG | 6053 | CCUCU UCAUC CCAUGCACUAUCC CCC ACUUCUUCCA | 15393 |
| 6844 | AACUCACACC C ACAUC | 6054 | CAUCU UCAUC GCAUCCACUAUGC CCC CCUGUCACUU | 15394 |
| 6849 | ACACCCACAU C UUAAU | 6055 | AUUAA UCAUC CCAUCCACUAUCC CCC AUCUCCCUCU | 15395 |
| 6868 | ACCCACCCCU C CCUUC | 6056 | CAACC UCAUC CCAUCCACUAUCC CCC ACCCCUCCCU | 15396 |
| 6873 | CCCCUCCCUU C UUUCC | 6057 | CCAAA UCAUC CCAUCCACUAUCC CCC AACCCACCCC | 15397 |
| 6893 | CCACAAGAAU C CAAAC | 6058 | CUCUC UCAUC CCAUCCACUAUCC CCC AUUCUUCUCC | 15398 |
| 6914 | ACACAUACUC C CUACC | 6059 | CCUAC UCAUC CCAUCCACUAUCC CCC CACUAUCUCU | 15399 |
| 6932 | CAUUUAAAUU C AUUAA | 6060 | UUAAU UCAUC CCAUCCACUAUCC CCC AAUUUAAAUC | 15400 |
| 6947 | AACCACCACU C CAUCU | 6061 | ACAUC UCAUC CCAUCCACUAUCC CCG ACUCCUCCUC | 15401 |
| 6959 | AUCUUUCCCC C ACACU | 6062 | ACUCU UCAUC CCAUCCACUAUCC CCC CCCCAAACAU | 15402 |
| 6968 | CCACACUCCU C UAACU | 6063 | ACUCA UCAUC CCAUCCACUAUCC CCC ACCACUCUCC | 15403 |
| 6974 | UCCUCUAACU C UCUGU | 6064 | ACACA UCAUC CCAUCCACUAUCC CCC ACUUACACCA | 15404 |
| 6976 | CUGUAACUCU C UCUCU | 6065 | ACACA UGAUC CCAUCCACUAUCC CCG ACACUCACAC | 15405 |
| 6978 | GUAACUCUCU C UCUCU | 6066 | ACACA UCAUC CCAUCCACUAUCC CCC ACACAGUCAC | 15406 |
| 6980 | AACUCUCUCU C UCUCU | 6067 | ACACA UCAUC CCAUCCACUAUCC CCC ACACACACUC | 15407 |
| 6982 | CUCUCUCUCU C UGUCU | 6068 | ACACA UCAUC CCAUCCACUAUCC CCC ACACACACAC | 15408 |
| 6984 | CUCUCUCUCU C UCUCU | 6069 | ACACA UCAUC CCAUCCACUAUCC CCC ACACACACAC | 15409 |
| 6986 | CUCUCUCUCU C UCUCU | 6069 | ACACA UCAUC CCAUCCACUAUCC CCC ACACACACAC | 15409 |
| 6988 | GUCUCUCUCU C UCUCU | 6069 | ACACA UCAUC CCAUCCACUAUCC CCC ACACACACAC | 15409 |
| 6990 | CUCUCUCUCU C UCUCU | 6069 | ACACA UCAUC CCAUCCACUAUCC CCC ACACACACAC | 15409 |
| 6992 | CUCUCUCUCU C UCUCU | 6069 | ACACA UCAUC CCAUCCACUAUCC CCC ACACACACAC | 15409 |
| 6994 | GUGUGUGUGU G UGUGU | 6069 | ACACA UGAUG GCAUGCACUAUGC GCG ACACACACAC | 15409 |
| 6996 | GUGUGUGUGU C UGUGU | 6069 | ACACA UGAUG GCAUCCACUAUGC CCC ACACACACAC | 15409 |
| 6998 | GUGUCUCUCU C UGUCU | 6069 | ACACA UCAUG CCAUGCACUAUGC GCG ACACACACAC | 15409 |
| 7000 | GUGUGUCUGU G UGUCU | 6069 | ACACA UGAUG GCAUGCACUAUCC CCG ACACACACAC | 15409 |
| 7002 | GUGUGUGUGU C UGUGU | 6069 | ACACA UGAUG GCAUGCACUAUGC GCG ACACACACAC | 15409 |
| 7004 | GUGUGUGUCU G UGUGU | 6069 | ACACA UGAUC CCAUGCACUAUGC CCC ACACACACAC | 15409 |
| 7006 | GUGUGUGUGU G UGUCU | 6069 | ACACA UGAUG GCAUCCACUAUGC CCG ACACACACAC | 15409 |
| 7008 | GUGUGUGUCU G UGUGU | 6069 | ACACA UGAUC GCAUCCACUAUGC GCG ACACACACAC | 15409 |
| 701G | CUGUCUGUCU G UCUCC | 6070 | CCACA UCAUG CCAUCCACUAUGC CCG ACACACACAC | 15410 |
| 7012 | GUGUCUGUCU C UCGCU | 6071 | ACCCA UCAUG CCAUGCACUAUGC GCG ACACACACAC | 15411 |
| 7018 | CUGUCUGGGU G UCCGU | 6072 | ACCCA UCAUC GCAUGCACUAUCC GCC ACCCACACAC | 15412 |
| 7024 | GGCUCUGCCU C UAUGU | 6073 | ACAGA UGAUG GCAUGCACUAUCC CCC ACCCACACCC | 15413 |
| 7028 | GUGGGUGUAU C UCUGU | 6074 | ACACA UCAUC CCAUGCACUAUGC GCC AUACACCCAC | 15414 |
| 7030 | CGGUCUAUCU G UGUUU | 6075 | AAACA UCAUC GCAUCCACUAUGC GCG ACAUACACCC | 15415 |
| 7032 | CUCUAUCUGU G UUUUG | 6076 | CAAAA UGAUC GCAUCCACUAUGC CCG ACACAUACAC | 15416 |
| 7037 | UGUGUGUUUU G UCCAU | 6077 | AUCCA UGAUG GCAUCCACUAUGC GCG AAAACACACA | 15417 |
| 7039 | UGUCUUUUGU G CAUAA | 6078 | UUAUG UGAUG GCAUGCACUAUGC GCG ACAAAACACA | 15418 |
| 7099 | AACAAUAUAU C CUACA | 6079 | UCUAC UCAUC CCAUCCACUAUCC CCC AUAUAUUCUU | 15419 |
| 7146 | UUCUACUCAU C AUCAA | 6080 | UUCAU UCAUC CCAUCCACUAUCC CCC AUCACUACAA | 1542G |
| 7149 | UACUCAUCAU C AAUCU | 6081 | ACAUU UCAUC CCAUCCACUAUCC CCC AUCAUCACUA | 15421 |
| 7153 | CAUCAUGAAU G UAUUU | 6082 | AAAUA UCAUG CCAUCCACUAUCC CCC AUUCAUCAUC | 15422 |
| 7160 | AAUCUAUUUU C UAUAC | 6083 | CUAGA UCAUC CCAUGCACUAUCC CCC AAAAUACAUU | 15423 |
| 721G | AUUCCCAUUU C UAAUC | 6084 | CAUUA UCAUC GCAUGCACUAUGC CCC AAAUCCCAAU | 15424 |
| 7230 | CAACUUAAUU C AUAAA | 6085 | UUUAU UCAUG CCAUCCACUAUCC CCC AAUUAACUUC | 15425 |
| 7246 | CUUCCCAACU C CUUUU | 6086 | AAAAC UCAUC CCAUCCACUAUCC CCC ACUUCCCAAC | 15426 |
| 7254 | CUCCUUUUAU C GUCUC | 6087 | CACAA UCAUC CCAUCCACUAUCC CCC AUAAAACCAC | 15427 |
| 7259 | UUUAUCUUCU C UCUCC | 6088 | CCACA UCAUC CCAUCCACUAUCC CCC ACAACAUAAA | 15428 |
| 7343 | AUUUAUCCUU C UGUAC | 6089 | CUAAA UCAUC CCAUGCACUAUCC CCC AACCAUAAAU | 15429 |
| 7375 | CAAAAACUUU C AAAUC | 6090 | CAGUG UCAUC CCAUCCACUAUCC CCC AAACUUUUUC | 15430 |
| 7394 | UCAAAAAAUU C CUAAA | 6091 | UUUAC UCAUC CCAUCCACUAUCC CCC AAUUUUUUCA | 15431 |
| 7422 | AAAACUAAAU C UUACU | 6092 | ACUAA UCAUC CCAUCCACUAUCC CCC AUUUACUUUU | 15432 |
| 7434 | UACUUUACCU C AUUCU | 6093 | ACAAU UCAUC CCAUCCACUAUCC CCC ACCUAAACUA | 15433 |
| 7438 | UUACCUCAUU C UAUCC | 6094 | CCAUA UCAUC CCAUCCACUAUCC CCC AAUCACCUAA | 15434 |
| 7451 | UCGCCUUUUC C AACCU | 6095 | ACCUG UCAUC CCAUCCACUAUCC CCC CAAAACCCCA | 15435 |
| 7467 | UUCACUUUUU C UUUCU | 6096 | ACAAA UCAUC CCAUCCACUAUCC CCC AAAAACUCAA | 15436 |
| 7471 | CUUUUCUUUC U UUUUA | 6097 | UAAAA UCAUC CCAUCCACUAUCC CCC AAACAAAAAC | 15437 |
| 7491 | UUUCACAACU C UCUAA | 6098 | CUACA UCAUC CCAUCCACUAUCC CCC ACUUCUCAAA | 15438 |
| 7493 | UCACAACUGU C UAAAU | 6099 | AUUGA UCAUC CCAUCCACUAUCC CCC ACACUUCUCA | 15439 |
| 7500 | UCUCUAAAUU C CCAAU | 6100 | AUUCC UGAUC CCAUCCACUAUCC CCC AAUUUACACA | 15440 |
| 7513 | AAUAAUUCCU C UCCAU | 6101 | AUCCA UCAUC CCAUCCACUAUCC CCC ACCAAUUAUU | 15441 |
| 7519 | UCCUCUCCAU C AAAAU | 6102 | AUUUU UCAUC CCAUCCACUAUCC CCC AUCCACACCA | 15442 |
| 7525 | CCAUCAAAAU C CAAAU | 6103 | AUGUC UCAUC CCAUCCACUAUCC CCC AUUUUCAUCC | 15443 |
| 7539 | AUUAUCCACU C GAGAU | 6104 | AUCUA UCAUC CCAUCCACUAUCC CCC ACUCCAUAAU | 15444 |
| 7551 | ACAUAUAUUU C ACCAU | 6105 | AUCCU UCAUC CCAUCCACUAUCC CCC AAAUAUAUCU | 15445 |
| 7582 | CCCUACUUUU C CCUUU | 6106 | AAACC UCAUC CCAUCCACUAUCC CCC AAAACUACCC | 15446 |
| 7611 | AUUUCACCCU C AAUCU | 6107 | ACAUU UCAUC CCAUCCACUAUCC CCC ACCCUCAAAU | 15447 |
| 7615 | CACCCUCAAU C UCUCC | 6108 | CCACA UCAUC CCAUCCACUAUCC CCC AUUCACCCUC | 15448 |
| 7619 | CUCAAUCUCU C CCUAU | 6109 | AUACC UCAUC CCAUCCACUAUCC CCC ACACAUUCAC | 15449 |
| 7634 | UAUAUUCUCU C CUCUG | 6110 | AACAC UCAUC CCAUCCACUAUCC CCC ACACAAUAUA | 15450 |
| 7641 | UCUGCUCUUU G UAUUC | 6111 | GAAUA UGAUG GCAUGCACUAUGC GCG AAAGAGCAGA | 15451 |
| 7653 | AUUCUCCUAA U AACCC | 6112 | GGGUU UGAUG GCAUGCACUAUGC GCG AAAGGAGAAU | 15452 |
| 7672 | AAAACAUCCU G UGGCA | 6113 | UGCCA UGAUG GCAUGCACUAUGC GCG AGGAUGUUUU | 15453 |

TABLE XVI
Human KDR NCH Ribozyme and Target Sequences

| | | Seq ID | | Seq ID |
|---|---|---|---|---|
| Pos | Target | No | Ribozyme Sequence | No |
| 11 | CAAGGUGC U CCUGGCCG | 6114 | CGGCCAGC CUGAUGAGGCCGUUAGGCCGAA ICACCUUG | 15454 |
| 14 | GGUGCUGC U GGCCGUCG | 6115 | CGACCGCC CUGAUGAGGCCGUUAGGCCGAA ICAGCACC | 15455 |
| 18 | CUGCUGGC C GUCGCCCU | 6116 | AGGGCCAC CUGAUGAGGCCGUUAGGCCGAA ICCAGCAG | 15456 |
| 24 | GCCGUCGC C CUGUGGCU | 6117 | AGCCACAG CUGAUGAGGCCGUUAGGCCGAA ICGACGGC | 15457 |
| 25 | CCGUCGCC C UGUOCCUC | 6118 | GAGCCACA CUGAUGAGGCCGUUAGGCCGAA IGCGACGG | 15458 |
| 26 | CGUCGCCC U GUGGCUCU | 6119 | AGAGCCAC CUGAUGAGGCCGUUAGGCCGAA IGGCGACG | 15459 |
| 32 | CCUGUGGC U CUGCGUGG | 6120 | CCACGCAG CUGAUGAGGCCGUUAGGCCGAA ICCACAUG | 15460 |
| 34 | UGUGGCUC U UGGUGGAG | 6121 | CUCCACUC CUGAUGAGGCCGUUAGGCCGAA IAUCCACA | 15461 |
| 45 | GUGGAGAC C CGGGCCGC | 6122 | GCGGCCCG CUGAUGAGGCCGUUAGGCCGAA IUCUCCAC | 15462 |
| 46 | UGGAGACC C GGGCCGCC | 6123 | GGCGGCCC CUGAUGAGGCCGUUAGGCCGAA IGUCUCCA | 15463 |
| 51 | ACCCGGGC C GCCUCUGU | 6124 | ACAGAGGC CUGAUGAGGCCGUUAGGCCGAA ICCCGGGU | 15464 |
| 54 | CGGGCCGC C UCUGUGGG | 6125 | CCCACAGA CUGAUGAGGCCGUUAGGCCGAA ICGGCCCG | 15465 |
| 55 | GGGCCGCC U CUGUGGGU | 6126 | ACCCACAG CUGAUGAGGCCGUUAGGCCGAA IGCGGCCC | 15466 |
| 57 | GCCGCCUC U GUGGGUUU | 6127 | AAACCCAC CUGAUGAGGCCGUUAGGCCGAA IAGGCGGC | 15467 |
| 68 | GGGUUUGC C UAGUGUUU | 6128 | AAACACUA CUGAUGAGGCCGUUAGGCCGAA ICAAACCC | 15468 |
| 69 | GGUUUGCC U AGUGUUUC | 6129 | GAAACACU CUGAUGAGGCCGUUAGGCCGAA IGCAAACC | 15469 |
| 78 | AGUGUUUC U CUUGAUCU | 6130 | AGAUCAAG CUGAUGAGGCCGUUAGGCCGAA IAAACACU | 15470 |
| 80 | UGUUUCUC U UGAUCUGC | 6131 | GCAGAUCA CUGAUGAGGCCGUUAGGCCGAA IAGAAACA | 15471 |
| 86 | UCUUGAUC U GCCCAGGC | 6132 | GCCUGGGC CUGAUGAGGCCGUUAGGCCGAA IAUCAAGA | 15472 |
| 89 | UGAUCUGC C CAGGCUCA | 6133 | UGAGCCUG CUGAUGAGGCCGUUAGGCCGAA ICAGAUCA | 15473 |
| 90 | GAUCUGCC C AGGCUCAG | 6134 | CUGAGCCU CUGAUGAGGCCGUUAGGCCGAA ICCAGAUC | 15474 |
| 91 | AUCUGCCC A GGCUCAGC | 6135 | GCUGAGCC CUGAUGAGGCCGUUAGGCCGAA IGGCAGAU | 15475 |
| 95 | GCCCAGGC U CAGCAUAC | 6136 | GUAUGCUG CUGAUGAGGCCGUUAGGCCGAA ICCUGGGC | 15476 |
| 97 | CCAGGCUC A GCAUACAA | 6137 | UUGUAUGC CUGAUGAGGCCGUUAGGCCGAA IAGCCUGG | 15477 |
| 100 | GGCUCAGC A UACAAAAA | 6138 | UUUUUGUA CUGAUGAGGCCGUUAGGCCGAA ICUGAGCC | 15478 |
| 104 | CAGCAUAC A AAAAGACA | 6139 | UGUCUUUU CUGAUGAGGCCGUUAGGCCGAA IUAUGCUG | 15479 |
| 112 | AAAAAGAC A UACUUACA | 6140 | UGUAAGUA CUGAUGAGGCCGUUAGGCCGAA IUCUUUUU | 15480 |
| 116 | AGACAUAC U UACAAUUA | 6141 | UAAUUGUA CUGAUGAGGCCGUUAGGCCGAA IUAUGUCU | 15481 |
| 120 | AUACUUAC A AUUAAGGC | 6142 | GCCUUAAU CUGAUGAGGCCGUUAGGCCGAA IUAAGUAU | 15482 |
| 129 | AUUAAGGC U AAUACAAC | 6143 | GUUGUAUU CUGAUGAGGCCGUUAGGCCGAA ICCUUAAU | 15483 |
| 135 | GCUAAUAC A ACUCUUCA | 6144 | UGAAGAGU CUGAUGAGGCCGUUAGGCCGAA IUAUUAGC | 15484 |
| 138 | AAUACAAC U CUUCAAAU | 6145 | AUUUGAAG CUGAUGAGGCCGUUAGGCCGAA IUUGUAUU | 15485 |
| 140 | UACAACUC U UCAAAUUA | 6146 | UAAUUUGA CUGAUGAGGCCGUUAGGCCGAA IAGUUGUA | 15486 |
| 143 | AACUCUUC A AAUUACUU | 6147 | AAGUAAUU CUGAUGAGGCCGUUAGGCCGAA IAAGAGUU | 15487 |
| 150 | CAAAUUAC U UGCAGGGG | 6148 | CCCCUGCA CUGAUGAGGCCGUUAGGCCGAA IUAAUUUG | 15488 |
| 154 | UUACUUGC A GGGGACAG | 6149 | CUGUCCCC CUGAUGAGGCCGUUAGGCCGAA ICAAGUAA | 15489 |
| 161 | CAGGGGAC A GAGGGACU | 6150 | AGUCCCUC CUGAUGAGGCCGUUAGGCCGAA IUCCCCUG | 15490 |
| 169 | AGAGUGAC U UGGACUGG | 6151 | CCAGUCCA CUGAUGAGGCCGUUAGGCCGAA IUCCCUCU | 15491 |
| 175 | ACUUGGAC U GGCUUUGG | 6152 | CCAAAGCC CUGAUGAGGCCGUUAGGCCGAA IUCCAAGU | 15492 |
| 179 | GGACUGGC U UUGGCCCA | 6153 | UGGGCCAA CUGAUGAGGCCGUUAGGCCGAA ICCAGUCC | 15493 |
| 185 | GCUUUGGC C CAAUAAUC | 6154 | GAUUAUUG CUGAUGAGGCCGUUAGGCCGAA ICCAAAGC | 15494 |
| 186 | CUUUGGCC C AAUAAUCA | 6155 | UGAUUAUU CUGAUGAGGCCGUUAGGCCGAA IGCCAAAG | 15495 |
| 187 | UUUGGCCC A AUAAUCAG | 6156 | CUGAUUAU CUGAUGAGGCCGUUAGGCCGAA IGGCCAAA | 15496 |
| 194 | CAAUAAUC A GAGUGGCA | 6157 | UGCCACUC CUGAUGAGGCCGUUAGGCCGAA IAUUAUUG | 15497 |
| 202 | AGAGUGGC A GUGAGCAA | 6158 | UUGCUCAC CUGAUGAGGCCGUUAGGCCGAA ICCACUCU | 15498 |
| 209 | CAGUGAGC A AAGGGUGG | 6159 | CCACCCUU CUGAUGAGGCCGUUAGGCCGAA ICUCACUG | 15499 |
| 225 | GAGGUGAC U GAGUOCAG | 6160 | CUGCACUC CUGAUGAGGCCGUUAGGCCGAA IUCACCUC | 15500 |
| 232 | CUGAGUGC A GCGAUGGC | 6161 | GCCAUCGC CUGAUGAGGCCGUUAGGCCGAA ICACUCAG | 15501 |
| 241 | UCCAUGUC C UCUUCUGU | 6162 | ACAGAAGA CUGAUGAGGCCGUUAGGCCGAA ICCAUCGC | 15502 |
| 242 | CGAUGGCC U CUUCUGUA | 6163 | UACAGAAG CUGAUGAGGCCGUUAGGCCGAA IGCCAUCG | 15503 |
| 244 | AUGGCCUC U UCUGUAAG | 6164 | CUUACAGA CUGAUGAGGCCGUUAGGCCGAA IAGGCCAU | 15504 |
| 247 | GCCUCUUC U GUAAGACA | 6165 | UGUCUUAC CUGAUGAGGCCGUUAGGCCGAA IAAGAGGC | 15505 |
| 255 | UGUAAGAC A CUCACAAU | 6166 | AUUGUGAG CUGAUGAGGCCGUUAGGCCGAA IUCUUACA | 15506 |
| 257 | UAAGACAC U CACAAUUC | 6167 | GAAUUGUG CUGAUGAGGCCGUUAGGCCGAA IUGUCUUA | 15507 |
| 259 | AGACACUC A CAAUUCCA | 6168 | UGGAAUUG CUGAUGAGGCCGUUAGGCCGAA IAGUGUCU | 15508 |
| 261 | ACACUCAC A AUUCCAAA | 6169 | UUUGGAAU CUGAUGAGGCCGUUAGGCCGAA IUGAGUGU | 15509 |
| 266 | CACAAUUC C AAAAGUGA | 6170 | UCACUUUU CUGAUGAGGCCGUUAGGCCGAA IAAUUGUG | 15510 |
| 267 | ACAAUUCC A AAAGUGAU | 6171 | AUCACUUU CUGAUGAGGCCGUUAGGCCGAA IGAAUUGU | 15511 |
| 286 | GAAAUGAC A CUGGAGCC | 6172 | GGCUCCAG CUGAUGAGGCCGUUAGGCCGAA IUCAUUUC | 15512 |
| 288 | AAUGACAC U GGAGCCUA | 6173 | UAGGCUCC CUGAUGAGGCCGUUAGGCCGAA IUGUCAUU | 15513 |
| 294 | ACUGGAGC C UACAAGUG | 6174 | CACUUGUA CUGAUGAGGCCGUUAGGCCGAA ICUCCAGU | 15514 |
| 295 | CUGGAGCC U ACAAGUGC | 6175 | GCACUUGU CUGAUGAGGCCGUUAGGCCGAA IGCUCCAG | 15515 |
| 298 | GAGCCUAC A AGUGCUUC | 6176 | GAAGCACU CUGAUGAGGCCGUUAGGCCGAA IUAGGCUC | 15516 |
| 304 | ACAAGUGC U UCUACCGG | 6177 | CCGGUAGA CUGAUGAGGCCGUUAGGCCGAA ICACUUGU | 15517 |
| 307 | AGUGCUUC U ACCGGGAA | 6178 | UUCCCGGU CUGAUGAGGCCGUUAGGCCGAA IAAGCACU | 15518 |
| 310 | GCUUCUAC C GGGAAACU | 6179 | AGUUUCCC CUGAUGAGGCCGUUAGGCCGAA IUAGAAGC | 15519 |
| 318 | CGGGAAAC U GACUUGGC | 6180 | GCCAAGUC CUGAUGAGGCCGUUAGGCCGAA IUUUCCCG | 15520 |
| 322 | AAACUGAC U UGGCCUCG | 6181 | CGAGGCCA CUGAUGAGGCCGUUAGGCCGAA IUCAGUUU | 15521 |
| 327 | GACUUGGC C UCGGUCAU | 6182 | AUGACCGA CUGAUGAGGCCGUUAGGCCGAA ICCAAGUC | 15522 |
| 328 | ACUUGGCC U CGGUCAUU | 6183 | AAUGACCG CUGAUGAGGCCGUUAGGCCGAA IGCCAAGU | 15523 |
| 334 | CCUCGGUC A UUUAUGUC | 6184 | GACAUAAA CUGAUGAGGCCGUUAGGCCGAA IACCGAGG | 15524 |
| 343 | UUUAUGUC U AUGUUCAA | 6185 | UUGAACAU CUGAUGAGGCCGUUAGGCCGAA IACAUAAA | 15525 |
| 350 | CUAUGUUC A AGAUUACA | 6186 | UGUAAUCU CUGAUGAGGCCGUUAGGCCGAA IAACAUAG | 15526 |
| 358 | AAGAUUAC A GAUCUCCA | 6187 | UGGAGAUC CUGAUGAGGCCGUUAGGCCGAA IUAAUCUU | 15527 |
| 363 | UACAGAUC U CCAUUUAU | 6188 | AUAAAUGG CUGAUGAGGCCGUUAGGCCGAA IAUCUGUA | 15528 |
| 365 | CAGAUCUC C AUUUAUUG | 6189 | CAAUAAAU CUGAUGAGGCCGUUAGGCCGAA IAGAUCUG | 15529 |
| 366 | AGAUCUCC A UUUAUUGC | 6190 | GCAAUAAA CUGAUGAGGCCGUUAGGCCGAA IGAGAUCU | 15530 |
| 375 | UUUAUUGC U UCUGUUAG | 6191 | CUAACAGA CUGAUGAGGCCGUUAGGCCGAA ICAAUAAA | 15531 |
| 378 | AUUGCUUC U GUUAGUGA | 6192 | UCACUAAC CUGAUGAGGCCGUUAGGCCGAA IAAGCAAU | 15532 |
| 388 | UUAGUGAC C AACAUGGA | 6193 | UCCAUGUU CUGAUGAGGCCGUUAGGCCGAA IUCACUAA | 15533 |
| 389 | UAGUGACC A ACAUGGAG | 6194 | CUCCAUGU CUGAUGAGGCCGUUAGGCCGAA IGUCACUA | 15534 |
| 392 | UGACCAAC A UGGAGUCG | 6195 | CGACUCCA CUGAUGAGGCCGUUAGGCCGAA IUUGGUCA | 15535 |
| 406 | UCCUGUAC A UUACUGAG | 6196 | CUCAGUAA CUGAUGAGGCCGUUAGGCCGAA IUACACCA | 15536 |
| 411 | UACAUUAC U GAGAACAA | 6197 | UUGUUCUC CUGAUGAGGCCGUUAGGCCGAA IUAAUGUA | 15537 |
| 418 | CUGAGAAC A AAAACAAA | 6198 | UUUGUUUU CUGAUGAGGCCGUUAGGCCGAA IUUCUCAG | 15538 |
| 424 | ACAAAAAC A AAACUGUG | 6199 | CACAGUUU CUGAUGAGGCCGUUAGGCCGAA IUUUUUGU | 15539 |
| 429 | AACAAAAC U GUGGUGAU | 6200 | AUCACCAC CUGAUGAGGCCGUUAGGCCGAA IUUUUGUU | 15540 |
| 440 | GGUGAUUC C AUGUCUCG | 6201 | CGAGACAU CUGAUGAGGCCGUUAGGCCGAA IAAUCACC | 15541 |
| 441 | GUGAUUCC A UGUCUCUG | 6202 | CCGAGACA CUGAUGAGGCCGUUAGGCCGAA IGAAUCAC | 15542 |
| 446 | UCCAUGUC U CGGGUCCA | 6203 | UGGACCCG CUGAUGAGGCCGUUAGGCCGAA IACAUGGA | 15543 |
| 453 | CUCGGGUC C AUUUCAAA | 6204 | UUUGAAAU CUGAUGAGGCCGUUAGGCCGAA IACCCGAG | 15544 |
| 454 | UCGGGUCC A UUUCAAAU | 6205 | AUUUGAAA CUGAUGAGGCCGUUAGGCCGAA IGACCCGA | 15545 |
| 459 | UCCAUUUC A AAUCUCAA | 6206 | UUGAGAUU CUGAUGAGGCCGUUAGGCCGAA IAAAUGGA | 15546 |
| 464 | UUCAAAUC U CAACGUGU | 6207 | ACACGUUG CUGAUGAGGCCGUUAGGCCGAA IAUUUGAA | 15547 |
| 466 | CAAAUCUC A ACGUGUCA | 6208 | UGACACGU CUGAUGAGGCCGUUAGGCCGAA IAGAUUUG | 15548 |
| 474 | AACGUGUC A CUUUGUGC | 6209 | GCACAAAG CUGAUGAGGCCGUUAGGCCGAA TACACGUU | 15549 |
| 476 | CGUGUCAC U UUGUGCAA | 6210 | UUGCACAA CUGAUGAGGCCGUUAGGCCGAA IUGACACG | 15550 |
| 483 | CUUUGUGC A AGAUACCC | 6211 | GGGUAUCU CUGAUGAGGCCGUUAGGCCGAA ICACAAAG | 15551 |
| 490 | CAAGAUAC C CAGAAAAG | 6212 | CUUUUCUG CUGAUGAGGCCGUUAGGCCGAA IUAUCUUG | 15552 |
| 491 | AAGAUACC C AGAAAAGA | 6213 | UCUUUUCU CUGAUGAGGCCGUUAGGCCGAA IGUAUCUU | 15553 |
| 492 | AGAUACCC A GAAAAGAG | 6214 | CUCUUUUC CUGAUGAGGCCGUUAGGCCGAA IGGUAUCU | 15554 |
| 509 | AUUUGUUC C UGAUGGUA | 6215 | UACCAUCA CUGAUGAGGCCGUUAGGCCGAA IAACAAAU | 15555 |
| 510 | UUUGUUCC U GAUGGUAA | 6216 | UUACCAUC CUGAUGAGGCCGUUAGGCCGAA IGAACAAA | 15556 |
| 520 | AUGGUAAC A GAAUUUCC | 6217 | GGAAAUUC CUGAUGAGGCCGUUAGGCCGAA IUUACCAU | 15557 |
| 528 | AGAAUUUC C UGGGACAG | 6218 | CUGUCCCA CUGAUGAGGCCGUUAGGCCGAA IAAAUUCU | 15558 |
| 529 | GAAUUUCC U GGGACAGC | 6219 | GCUGUCCC CUGAUGAGGCCGUUAGGCCGAA IGAAAUUC | 15559 |
| 535 | CCUCGCAC A GCAAGAAG | 6220 | CUUCUUGC CUGAUGAGGCCGUUAGGCCGAA IUCCCAGG | 15560 |
| 538 | GGGACAGC A AGAAGGGC | 6221 | GCCCUUCU CUGAUGAGGCCGUUAGGCCGAA ICUGUCCC | 15561 |
| 547 | AGAACGGC U UUACUAUU | 6222 | AAUAGUAA CUGAUGAGGCCGUUAGGCCGAA ICCCUUCU | 15562 |
| 552 | GGCUUUAC U AUUCCCAG | 6223 | CUGGGAAU CUGAUGAGGCCGUUAGGCCGAA IUAAAGCC | 15563 |
| 557 | UACUAUUC C CACCUACA | 6224 | UGUAGCUG CUGAUGAGGCCGUUAGGCCGAA IAAUAGUA | 15564 |
| 558 | ACUAUUCC C AGCUACAU | 6225 | AUGUAGCU CUGAUGAGGCCGUUAGGCCGAA IGAAUACU | 15565 |
| 559 | CUAUUCCC A UCUACAUG | 6226 | CAUGUAGC CUGAUGAGGCCGUUAGGCCGAA IGGAAUAG | 15566 |
| 562 | UUCCCAGC U ACAUGAUC | 6227 | GAUCAUGU CUGAUGAGGCCGUUAGGCCGAA ICUGGGAA | 15567 |
| 565 | CCAGCUAC A UGAUCAUC | 6228 | GCUGAUCA CUGAUGAGGCCGUUAGGCCGAA IUAGCUGG | 15568 |
| 571 | ACAUGAUC A GCUAUGCU | 6229 | AGCAUAGC CUGAUGAGGCCGUUAGGCCGAA IAUCAUGU | 15569 |
| 574 | UGAUCAGC U AUGCUCGC | 6230 | GCCAGCAU CUGAUGAGGCCGUUAGGCCGAA ICUGAUCA | 15570 |
| 579 | AGCUAUGC U GGCAUGGU | 6231 | ACCAUGCC CUGAUGAGGCCGUUAGGCCGAA ICAUAGCU | 15571 |
| 583 | AUGCUGGC A UGGUCUUC | 6232 | GAAGACCA CUGAUGAGGCCGUUAGGCCGAA ICCAGCAU | 15572 |
| 589 | GCAUGGUC U UCUGUGAA | 6233 | UUCACAGA CUGAUGAGGCCGUUAGGCCGAA IACCAUGC | 15573 |
| 592 | UGGUCUUC U GUGAAGCA | 6234 | UGCUUCAC CUGAUGAGGCCGUUAGGCCGAA IAAGACCA | 15574 |
| 600 | UGUGAAGC A AAAAUUAA | 6235 | UUAAUUUU CUGAUGAGGCCGUUAGGCCGAA ICUUCACA | 15575 |
| 622 | AAAGUUAC C AGUCUAUU | 6236 | AAUAGACU CUGAUGAGGCCGUUAGGCCGAA IUAACUUU | 15576 |
| 623 | AAGUUACC A GUCUAUUA | 6237 | UAAUAGAC CUGAUGAGGCCGUUAGGCCGAA IGUAACUU | 15577 |
| 627 | UACCAGUC U AUUAUGUA | 6238 | UACAUAAU CUGAUGAGGCCGUUAGGCCGAA IACUGGUA | 15578 |
| 637 | UUAUGUAC A UAGUUGUC | 6239 | GACAACUA CUGAUGAGGCCGUUAGGCCGAA IUACAUAA | 15579 |
| 677 | UGUGGUUC U GAGUCCGU | 6240 | ACUGACUC CUGAUGAGGCCGUUAGGCCGAA IAACCACA | 15580 |
| 683 | UCUGAGUC C GUCUCAUG | 6241 | CAUGAGAC CUGAUGAGGCCGUUAGGCCGAA IACUCAGA | 15581 |
| 687 | AGUCCGUC U CAUGGAAU | 6242 | AUUCCAUG CUGAUGAGGCCGUUAGGCCGAA IACGGACU | 15582 |
| 689 | UCCGUCUC A UGGAAUUG | 6243 | CAAUUCCA CUGAUGAGGCCGUUAGGCCGAA IAGACGGA | 15583 |
| 701 | AAUUGAAC U AUCUGUUG | 6244 | CAACAGAU CUGAUGAGGCCGUUAGGCCGAA IUUCAAUU | 15584 |
| 705 | GAACUAUC U GUUGGAGA | 6245 | UCUCCAAC CUGAUGAGGCCGUUAGGCCGAA IAUAGUUC | 15585 |
| 719 | AGAAAAGC U UGUCUUAA | 6246 | UUAAGACA CUGAUGAGGCCGUUAGGCCGAA ICUUUUCU | 15586 |
| 724 | AGCUUGUC U UAAAUUGU | 6247 | ACAAUUUA CUGAUGAGGCCGUUAGGCCGAA IACAAGCU | 15587 |
| 735 | AAUUGUAC A GCAAGAAC | 6248 | GUUCUUGC CUGAUGAGGCCGUUAGGCCGAA IUACAAUU | 15588 |
| 738 | UGUACAGC A AGAACUGA | 6249 | UCAGUUCU CUGAUGAGGCCGUUAGGCCGAA ICUGUACA | 15589 |
| 744 | GCAAGAAC U GAACUAAA | 6250 | UUUAGUUC CUGAUGAGGCCGUUAGGCCGAA IUUCUUGC | 15590 |
| 749 | AACUGAAC U AAAUGUGG | 6251 | CCACAUUU CUGAUGAGGCCGUUAGGCCGAA IUUCAGUU | 15591 |
| 766 | GGAUUGAC U UCAACUGG | 6252 | CCAGUUGA CUGAUGAGGCCGUUAGGCCGAA IUCAAUCC | 15592 |
| 769 | UUGACUUC A ACUGGGAA | 6253 | UUCCCAGU CUGAUGAGGCCGUUAGGCCGAA IAAGUCAA | 15593 |
| 772 | ACUUCAAC U GGGAAUAC | 6254 | GUAUUCCC CUGAUGAGGCCGUUAGGCCGAA IUUGAAGU | 15594 |
| 781 | GGGAAUAC C CUUCUUCG | 6255 | CGAAGAAG CUGAUGAGGCCGUUAGGCCGAA IUAUUCCC | 15595 |
| 782 | GGAAUACC C UUCUUCGA | 6256 | UCGAAGAA CUGAUGAGGCCGUUAGGCCGAA IGUAUUCC | 15596 |
| 783 | GAAUACCC U UCUUCGAA | 6257 | UUCGAAGA CUGAUGAGGCCGUUAGGCCGAA IGGUAUUC | 15597 |
| 786 | UACCCUUC U UCGAACCA | 6258 | UGCUUCGA CUGAUGAGGCCGUUAGGCCGAA IAAGGGUA | 15598 |
| 794 | UUCGAAGC A UCAGCAUA | 6259 | UAUGCUGA CUGAUGAGGCCGUUAGGCCGAA ICUUCCAA | 15599 |
| 797 | GAAGCAUC A GCAUAAGA | 6260 | UCUUAUGC CUGAUGAGGCCGUUAGGCCGAA IAUGCUUC | 15600 |
| 800 | GCAUCAGC A UAAGAAAC | 6261 | GUUUCUUA CUGAUGAGGCCGUUAGGCCGAA ICUGAUGC | 15601 |
| 809 | UAAGAAAC U UGUAAACC | 6262 | GGUUUACA CUGAUGAGGCCGUUAGGCCGAA IUUUCUUA | 15602 |
| 817 | UUGUAAAC C GAGACCUA | 6263 | UAGGUCUC CUGAUGAGGCCGUUAGGCCGAA IUUUACAA | 15603 |
| 823 | ACCGACAC C UAAAAACC | 6264 | GGUUUUUA CUGAUGAGGCCGUUAGGCCGAA IUCUCGGU | 15604 |
| 824 | CCCAGACC U AAAAACCC | 6265 | GGGUUUUU CUGAUGAGGCCGUUAGGCCGAA ICUCUCGG | 15605 |
| 831 | CUAAAAAC C CAGUCUGG | 6266 | CCAGACUG CUGAUGAGGCCGUUAGGCCGAA IUUUUUAG | 15606 |
| 832 | UAAAAACC C AGUCUGGG | 6267 | CCCAGACU CUGAUGAGGCCGUUAGGCCGAA IGUUUUUA | 15607 |
| 833 | AAAAACCC A GUCUGGGA | 6268 | UCCCAGAC CUGAUGAGGCCGUUAGGCCGAA IGGUUUUU | 15608 |
| 837 | ACCCAGUC U GGGAGUGA | 6269 | UCACUCCC CUGAUGAGGCCGUUAGGCCGAA IACUCGCU | 15609 |
| 865 | UUUUGAGC A CCUUAACU | 6270 | AGUUAAGG CUGAUGAGGCCGUUAGGCCGAA ICUCAAAA | 15610 |
| 867 | UUGAGCAC C UUAACUAU | 6271 | AUAGUUAA CUGAUGAGGCCGUUAGGCCGAA IUGCUCAA | 15611 |
| 868 | UCAGCACC U UAACUAUA | 6272 | UAUAGUUA CUGAUGAGGCCGUUAGGCCGAA IGUGCUCA | 15612 |
| 873 | ACCUUAAC U AUAGAUGC | 6273 | CCAUCUAU CUGAUGAGGCCGUUAGGCCGAA IUUAAGGU | 15613 |
| 888 | GGUCUAAC C CGGAGUGA | 6274 | UCACUCCG CUGAUGAGGCCGUUAGGCCGAA IUUACACC | 15614 |
| 889 | GUGUAACC C GUAGUGAC | 6275 | GUCACUCC CUGAUGAGGCCGUUAGGCCGAA IGUUACAC | 15615 |
| 898 | GGAGUGAC C AAGGAUUG | 6276 | CAAUCCUU CUGAUGAGGCCGUUAGGCCGAA IUCACUCC | 15616 |
| 899 | GAGUGACC A AGGAUUGU | 6277 | ACAAUCCU CUGAUGAGGCCGUUAGGCCGAA IGUCACUC | 15617 |
| 910 | GAUUGUAC A CCUGUGCA | 6278 | UGCACAGG CUGAUGAGGCCGUUAGGCCGAA IUACAAUC | 15618 |
| 912 | UUGUACAC C UGUGCAGC | 6279 | GCUGCACA CUGAUGAGGCCGUUAGGCCGAA IUGUACAA | 15619 |
| 913 | UGUACACC U GUGCAGCA | 6280 | UGCUGCAC CUGAUGAGGCCGUUAGGCCGAA IGUGUACA | 15620 |
| 918 | ACCUGUGC A GCAUCCAG | 6281 | CUGGAUGC CUGAUGAGGCCGUUAGGCCGAA ICACAGGU | 15621 |
| 921 | UGUGCAGC A UCCAGUOG | 6282 | CCACUGGA CUGAUGAGGCCGUUAGGCCGAA ICUCCACA | 15622 |
| 924 | GCACCAUC C AGUGGGCU | 6283 | AGCCCACU CUGAUGAGGCCGUUAGGCCGAA IAUGCUGC | 15623 |
| 925 | CAGCAUCC A GUGGGCUG | 6284 | CAGCCCAC CUGAUGAGGCCGUUAGGCCGAA IGAUGCUG | 15624 |
| 932 | CAGUGGGC U GAUGACCA | 6285 | UGGUCAUC CUGAUGAGGCCGUUAGGCCGAA ICCCACUG | 15625 |
| 939 | CUGAUGAC C AAGAAGAA | 6286 | UUCUUCUU CUGAUGAGGCCGUUAGGCCGAA IUCAUCAG | 15626 |
| 940 | UGAUGACC A AGAAGAAC | 6287 | GUUCUUCU CUGAUGAGGCCGUUAGGCCGAA IGUCAUCA | 15627 |
| 949 | AGAAGAAC A GCACAUUU | 6288 | AAAUGUGC CUGAUGAGGCCGUUAGGCCGAA IUUCUUCU | 15628 |
| 952 | AGAACAGC A CAUUUGUC | 6289 | GACAAAUG CUGAUGAGGCCGUUAGGCCGAA ICUGUUCU | 15629 |
| 954 | AACAGCAC A UUUGUCAG | 6290 | CUGACAAA CUGAUGAGGCCGUUAGGCCGAA IUGCUGUU | 15630 |
| 961 | CAUUUGUC A GGGUCCAU | 6291 | AUGGACCC CUGAUGAGGCCGUUAGGCCGAA IACAAAUG | 15631 |
| 967 | UCAGGGUC C AUGAAAAA | 6292 | UUUUUCAU CUGAUGAGGCCGUUAGGCCGAA IACCCUGA | 15632 |
| 968 | CAGGGUCC A UGAAAAAC | 6293 | GUUUUUCA CUGAUGAGGCCGUUAGGCCGAA IGACCCUG | 15633 |
| 977 | UGAAAAAC C UUUUGUUG | 6294 | CAACAAAA CUGAUGAGGCCGUUAGGCCGAA IUUUUUCA | 15634 |
| 978 | GAAAAACC U UUUCUUCC | 6295 | GCAACAAA CUGAUGAGGCCGUUAGGCCGAA IGUUUUUC | 15635 |
| 987 | UUUGUUGC U UUUGGAAG | 6296 | CUUCCAAA CUGAUGAGGCCGUUAGGCCGAA ICAACAAA | 15636 |
| 1000 | GAAGUGGC A UGGAAUCU | 6297 | AGAUUCCA CUGAUGAGGCCGUUAGGCCGAA ICCACUUC | 15637 |
| 1008 | AUGGAAUC U CUGGUGGA | 6298 | UCCACCAG CUGAUGAGGCCGUUAGGCCGAA IAUUCCAU | 15638 |
| 1010 | GGAAUCUC U GGUGGAAG | 6299 | CUUCCACC CUGAUGAGGCCGUUAGGCCGAA IAGAUUCC | 15639 |
| 1020 | GUGGAAGC C ACGGUGGG | 6300 | CCCACCGU CUGAUGAGGCCGUUAGGCCGAA ICUUCCAC | 15640 |
| 1021 | UGGAAGCC A CGGUGGGG | 6301 | CCCCACCG CUGAUGAGGCCGUUAGGCCGAA IGCUUCCA | 15641 |
| 1039 | AGCGUGUC A GAAUCCCU | 6302 | AGGGAUUC CUGAUGAGGCCGUUAGGCCGAA IACACGCU | 15642 |
| 1045 | UCAGAAUC C CUGCGAAG | 6303 | CUUCGCAG CUGAUGAGGCCGUUAGGCCGAA IAUUCUGA | 15643 |
| 1046 | CAGAAUCC C UGCGAAGU | 6304 | ACUUCGCA CUGAUGAGGCCGUUAGGCCGAA IGAUUCUG | 15644 |
| 1047 | AGAAUCCC U GCGAAGUA | 6305 | UACUUCGC CUGAUGAGGCCGUUAGGCCGAA IGGAUUCU | 15645 |
| 1057 | CGAAGUAC C UUGGUUAC | 6306 | GUAACCAA CUGAUGAGGCCGUUAGGCCGAA IUACUUCG | 15646 |
| 1058 | GAAGUACC U UGGUUACC | 6307 | GGUAACCA CUGAUGAGGCCGUUAGGCCGAA IGUACUUC | 15647 |
| 1066 | UUGGUUAC C CACCCCCA | 6308 | UCGGGGUG CUGAUGAGGCCGUUAGGCCGAA IUAACCAA | 15648 |
| 1067 | UGGUUACC C ACCCCCAG | 6309 | CUGGGCCU CUGAUGAGGCCGUUAGGCCGAA IGUAACCA | 15649 |
| 1068 | GGUUACCC A CCCCCAOA | 6310 | UCUGGGGG CUGAUGAGGCCGUUAGGCCGAA ICGUAACC | 15650 |
| 1070 | UUACCCAC C CCCAGAAA | 6311 | UUUCUGGG CUGAUGAGGCCGUUAGGCCGAA IUGGGUAA | 15651 |
| 1071 | UACCCACC C CCAGAAAU | 6312 | AUUUCUGC CUGAUGAGGCCGUUAGGCCGAA IGUGGGUA | 15652 |
| 1072 | ACCCACCC C CAGAAAUA | 6313 | UAUUUCUG CUGAUGAGGCCGUUAGGCCGAA IGGUGGGU | 15653 |
| 1073 | CCCACCCC C AGAAAUAA | 6314 | UUAUUUCU CUGAUGAGGCCGUUAGGCCGAA IGGCUGGG | 15654 |
| 1074 | CCACCCCC A GAAAUAAA | 6315 | UUUAUUUC CUGAUGAGGCCGUUAGGCCGAA ICGGGUGG | 15655 |
| 1103 | UGGAAUAC C CCUUGACU | 6316 | ACUCAAGG CUGAUGAGGCCGUUAGGCCGAA IUAUUCCA | 15656 |
| 1104 | GGAAUACC C CUUGAGUC | 6317 | GACUCAAG CUGAUGAGGCCGUUAGGCCGAA IGUAUUCC | 15657 |
| 1105 | GAAUACCC C UUGAGUCC | 6318 | GGACUCAA CUGAUGAGGCCGUUAGGCCGAA IGGUAUUC | 15658 |
| 1106 | AAUACCCC U UGAGUCCA | 6319 | UGGACUCA CUGAUGAGGCCGUUAGGCCGAA IGGGUAUU | 15659 |
| 1113 | CUUGAGUC C AAUCACAC | 6320 | GUGUGAUU CUGAUGAGGCCGUUAGGCCGAA IACUCAAG | 15660 |
| 1114 | UUGAGUCC A AUCACACA | 6321 | UGUGUGAU CUGAUGAGGCCGUUAGGCCGAA IGACUCAA | 15661 |
| 1118 | GUCCAAUC A CACAAUUA | 6322 | UAAUUGUG CUGAUGAGGCCGUUAGGCCGAA IAUUGGAC | 15662 |
| 1120 | CCAAUCAC A CAAUUAAA | 6323 | UUUAAUUG CUGAUGAGGCCGUUAGGCCGAA IUGAUUGG | 15663 |
| 1122 | AAUCACAC A AUUAAAGC | 6324 | GCUUUAAU CUGAUGAGGCCGUUAGGCCGAA IUGUGAUU | 15664 |
| 1136 | AGCGGGGC A UGUACUGA | 6325 | UCAGUACA CUGAUGAGGCCGUUAGGCCGAA ICCCCGCU | 15665 |
| 1142 | OCAUGUAC U GACGAUUA | 6326 | UAAUCGUC CUGAUGAGGCCGUUAGGCCGAA IUACAUGC | 15666 |
| 1171 | AAAGAGAC A CAGGAAAU | 6327 | AUUUCCUG CUGAUGAGGCCGUUAGGCCGAA IUCUCUUU | 15667 |
| 1173 | AGAGACAC A GGAAAUUA | 6328 | UAAUUUCC CUGAUGAGGCCGUUAGGCCGAA IUGUCUCU | 15668 |
| 1183 | GAAAUUAC A CUGUCAUC | 6329 | GAUGACAG CUGAUGAGGCCGUUAGGCCGAA IUAAUUUC | 15669 |
| 1185 | AAUUACAC U GUCAUCCU | 6330 | AGGAUGAC CUGAUGAGGCCGUUAGGCCGAA IUGUAAUU | 15670 |
| 1189 | ACACUGUC A UCCUUACC | 6331 | GGUAAGGA CUGAUGAGGCCGUUAGGCCGAA IACAGUGU | 15671 |
| 1192 | CUGUCAUC C UUACCAAU | 6332 | AUUGGUAA CUGAUGAGGCCGUUAGGCCGAA IAUGACAG | 15672 |
| 1193 | UGUCAUCC U UACCAAUC | 6333 | GAUUGGUA CUGAUGAGGCCGUUAGGCCGAA IGAUGACA | 15673 |
| 1197 | AUCCUUAC C AAUCCCAU | 6334 | AUGGGAUU CUGAUGAGGCCGUUAGGCCGAA IUAAGGAU | 15674 |
| 1198 | UCCUUACC A AUCCCAUU | 6335 | AAUGGGAU CUGAUGAGGCCGUUAGGCCGAA IGUAAGGA | 15675 |
| 1202 | UACCAAUC C CAUUUCAA | 6336 | UUGAAAUG CUGAUGAGGCCGUUAGGCCGAA IAUUGGUA | 15676 |
| 1203 | ACCAAUCC C AUUUCAAA | 6337 | UUUGAAAU CUGAUGAGGCCGUUAGGCCGAA IGAUUGGU | 15677 |
| 1204 | CCAAUCCC A UUUCAAAG | 6338 | CUUUGAAA CUGAUGAGGCCGUUAGGCCGAA IGGAUUGG | 15678 |
| 1209 | CCCAUUUC A AAGGAGAA | 6339 | UUCUCCUU CUGAUGAGGCCGUUAGGCCGAA IAAAUGGG | 15679 |
| 1220 | GGAGAAGC A GAGCCAUG | 6340 | CAUGGCUC CUGAUGAGGCCGUUAGGCCGAA ICUUCUCC | 15680 |
| 1225 | AGCAGAGC C AUGUGGUC | 6341 | GACCACAU CUGAUGAGGCCGUUAGGCCGAA ICUCUGCU | 15681 |
| 1226 | GCAGAGCC A UGUGGUCU | 6342 | AGACCACA CUGAUGAGGCCGUUAGGCCGAA IGCUCUGC | 15682 |
| 1234 | AUGUGGUC U CUCUGGUU | 6343 | AACCAGAG CUGAUGAGGCCGUUAGGCCGAA IACCACAU | 15683 |
| 1236 | GUGGUCUC U CUGGUUGU | 6344 | ACAACCAG CUGAUGAGGCCGUUAGGCCGAA IAGACCAC | 15684 |
| 1238 | GGUCUCUC U GGUUGUGU | 6345 | ACACAACC CUGAUGAGGCCGUUAGGCCGAA IAGAGACC | 15685 |
| 1252 | UGUAUGUC C CACCCCAG | 6346 | CUGGGGUG CUGAUGAGGCCGUUAGGCCGAA IACAUACA | 15686 |
| 1253 | GUAUGUCC C ACCCCAGA | 6347 | UCUGGGGU CUGAUGAGGCCGUUAGGCCGAA IGACAUAC | 15687 |
| 1254 | UAUGUCCC A CCCCAGAU | 6348 | AUCUGGGG CUGAUGAGGCCGUUAGGCCGAA IGGACAUA | 15688 |
| 1256 | UGUCCCAC C CCAGAUUG | 6349 | CAAUCUGG CUGAUGAGGCCGUUAGGCCGAA IUGGGACA | 15689 |
| 1257 | GUCCCACC C CAGAUUGG | 6350 | CCAAUCUG CUGAUGAGGCCGUUAGGCCGAA IGUGGGAC | 15690 |
| 1258 | UCCCACCC C AGAUUGGU | 6351 | ACCAAUCU CUGAUGAGGCCGUUAGGCCGAA IGGUGGGA | 15691 |
| 1259 | CCCACCCC A GAUUGGUG | 6352 | CACCAAUC CUGAUGAGGCCGUUAGGCCGAA IGGGUGGG | 15692 |
| 1275 | GAGAAAUC U CUAAUCUC | 6353 | GAGAUUAG CUGAUGAGGCCGUUAGGCCGAA IAUUUCUC | 15693 |
| 1277 | GAAAUCUC U AAUCUCUC | 6354 | GAGAGAUU CUGAUGAGGCCGUUAGGCCGAA IAGAUUUC | 15694 |
| 1282 | CUCUAAUC U CUCCUGUG | 6355 | CACAGGAG CUGAUGAGGCCGUUAGGCCGAA IAUUAGAG | 15695 |
| 1284 | CUAAUCUC U CCUGUGGA | 6356 | UCCACAGG CUGAUGAGGCCGUUAGGCCGAA IAGACUAG | 15696 |
| 1286 | AAUCUCUC C UGUGGAUU | 6357 | AAUCCACA CUGAUGAGGCCGUUAGGCCGAA IAGAGAUU | 15697 |
| 1287 | AUCUCUCC U GUGGAUUC | 6358 | GAAUCCAC CUGAUGAGGCCGUUAGGCCGAA IGAGAGAU | 15698 |
| 1296 | GUGGAUUC C UACCAGUA | 6359 | UACUGGUA CUGAUGAGGCCGUUAGGCCGAA IAAUCCAC | 15699 |
| 1297 | UGGAUUCC U ACCAGUAC | 63 | GUACUGGU CUGAUGAGGCCGUUAGGCCGAA IGAAUCCA | 15700 |
| 1300 | AUUCCUAC C AGUACCOC | 6361 | GCCGUACU CUGAUGAGGCCGUUAGGCCGAA IUAGGAAU | 15701 |
| 1301 | UUCCUACC A GUACGGCA | 6362 | UGCCGUAC CUGAUGAGGCCGUUAGGCCGAA IGUAGGAA | 15702 |
| 1309 | AGUACCOC A CCACUCAA | 6363 | UUGAGUGG CUGAUGAGGCCGUUAGGCCGAA ICCGUACU | 15703 |
| 1311 | UACGGCAC C ACUCAAAC | 6364 | CUUUGAGU CUGAUGAGGCCGUUAGGCCGAA IUGCCGUA | 15704 |
| 1312 | ACGGCACC A CUCAAACG | 6365 | CGUUUGAG CUGAUGAGGCCGUUAGGCCGAA IGUGCCGU | 15705 |
| 1314 | GGCACCAC U CAAACGCU | 6366 | AGCGUUUG CUGAUGAGGCCGUUAGGCCGAA IUGGUGCC | 15706 |
| 1316 | CACCACUC A AACGCUGA | 6367 | UCAGCGUU CUGAUGAGGCCGUUAGGCCGAA IAGUGGUG | 15707 |
| 1322 | UCAAACGC U GACAUGUA | 6368 | UACAUGUC CUGAUGAGGCCGUUAGGCCGAA ICGUUUGA | 15708 |
| 1326 | ACGCUGAC A UGUACGGU | 6369 | ACCGUACA CUGAUGAGGCCGUUAGGCCGAA IUCAGCGU | 15709 |
| 1336 | GUACGGUC U AUGCCAUU | 6370 | AAUGGCAU CUGAUGAGGCCGUUAGGCCGAA IACCGUAC | 15710 |
| 1341 | GUCUAUGC C AUUCCUCC | 6371 | GGAGGAAU CUGAUGAGGCCGUUAGGCCGAA ICAUAGAC | 15711 |
| 1342 | UCUAUGCC A UUCCUCCC | 6372 | GGGAGGAA CUGAUGAGGCCGUUAGGCCGAA IGCAUAGA | 15712 |
| 1346 | UGCCAUUC C UCCCCCGC | 6373 | GCGGGGGA CUGAUGAGGCCGUUAGGCCGAA IAAUGGCA | 15713 |
| 1347 | GCCAUUCC U CCCCCGCA | 6374 | UGCGGGGG CUGAUGAGGCCGUUAGGCCGAA IGAAUGGC | 15714 |
| 1349 | CAUUCCUC C CCCGCAUC | 6375 | GAUGCGGG CUGAUGAGGCCGUUAGGCCGAA IAGGAAUG | 15715 |
| 1350 | AUUCCUCC C CCGCAUCA | 6376 | UGAUGCGG CUGAUGAGGCCGUUAGGCCGAA IGAGGAAU | 15716 |
| 1351 | UUCCUCCC C CGCAUCAC | 6377 | GUGAUGCG CUGAUGAGGCCGUUAGGCCGAA IGGAGGAA | 15717 |
| 1352 | UCCUCCCC C GCAUCACA | 6378 | UGUGAUGC CUGAUGAGGCCGUUAGGCCGAA IGGGAGGA | 15718 |
| 1355 | UCCCCCGC A UCACAUCC | 6379 | GGAUGUGA CUGAUGAGGCCGUUAGGCCGAA ICGGGGGA | 15719 |
| 1358 | CCCGCAUC A CAUCCACU | 6380 | AGUGGAUG CUGAUGAGGCCGUUAGGCCGAA IAUGCGGG | 15720 |
| 1360 | CGCAUCAC A UCCACUGG | 6381 | CCAGUGGA CUGAUGAGGCCGUUAGGCCGAA IUGAUCCG | 15721 |
| 1363 | AUCACAUC C ACUGGUAU | 6382 | AUACCAGU CUGAUGAGGCCGUUAGGCCGAA IAUGUGAU | 15722 |
| 1364 | UCACAUCC A CUGGUAUU | 6383 | AAUACCAG CUGAUGAGGCCGUUAGGCCGAA IGAUGUGA | 15723 |
| 1366 | ACAUCCAC U GGUAUUGG | 6384 | CCAAUACC CUGAUGAGGCCGUUAGGCCGAA IUGGAUGU | 15724 |
| 1376 | GUAUUGGC A GUUGGAGG | 6385 | CCUCCAAC CUGAUGAGGCCGUUAGGCCGAA ICCAAUAC | 15725 |
| 1395 | GAGUGCGC C AACGAGCC | 6386 | GGCUCGUU CUGAUGAGGCCGUUAGGCCGAA ICGCACUC | 15726 |
| 1396 | AGUGCGCC A ACGAGCCC | 6387 | GGGCUCGU CUGAUGAGGCCGUUAGGCCGAA IGCGCACU | 15727 |
| 1403 | CAACGAGC C CAGCCAAG | 6388 | CUUGGCUG CUGAUGAGGCCGUUAGGCCGAA ICUCGUUG | 15728 |
| 1404 | AACGAGCC C AGCCAAGC | 6389 | GCUUGGCU CUGAUGAGGCCGUUAGGCCGAA IGCUCGUU | 15729 |
| 1405 | ACGAGCCC A GCCAAGCU | 6390 | AGCUUGGC CUGAUGAGGCCGUUAGGCCGAA IGGCUCGU | 15730 |
| 1408 | AGCCCAGC C AAGCUGUC | 6391 | GACAGCUU CUGAUGAGGCCGUUAGGCCGAA ICUGGGCU | 15731 |
| 1409 | GCCCAGCC A AGCUGUCU | 6392 | AGACAGCU CUGAUGAGGCCGUUAGGCCGAA IGCUGGGC | 15732 |
| 1413 | AGCCAAGC U GUCUCAGU | 6393 | ACUGAGAC CUGAUGAGGCCGUUAGGCCGAA ICUUGGCU | 15733 |
| 1417 | AAGCUGUC U CAGUGACA | 6394 | UGUCACUG CUGAUGAGGCCGUUAGGCCGAA IACAGCUU | 15734 |
| 1419 | GCUGUCUC A GUGACAAA | 6395 | UUUGUCAC CUGAUGAGGCCGUUAGGCCGAA IAGACAGC | 15735 |
| 1425 | UCAGUGAC A AACCCAUA | 6396 | UAUGGGUU CUGAUGAGGCCGUUAGGCCGAA IUCACUGA | 15736 |
| 1429 | UGACAAAC C CAUACCCU | 6397 | AGGGUAUG CUGAUGAGGCCGUUAGGCCGAA IUUUGUCA | 15737 |
| 1430 | GACAAACC C AUACCCUU | 6398 | AAGGGUAU CUGAUGAGGCCGUUAGGCCGAA IGUUUGUC | 15738 |
| 1431 | ACAAACCC A UACCCUUG | 6399 | CAAGGGUA CUGAUGAGGCCGUUAGGCCGAA IGGUUUGU | 15739 |
| 1435 | ACCCAUAC C CUUGUGAA | 6400 | UUCACAAG CUGAUGAGGCCGUUAGGCCGAA IUAUGGGU | 15740 |
| 1436 | CCCAUACC C UUGUGAAG | 6401 | CUUCACAA CUGAUGAGGCCGUUAGGCCGAA IGUAUGGG | 15741 |
| 1437 | CCAUACCC U UGUGAAGA | 6402 | UCUUCACA CUGAUGAGGCCGUUAGGCCGAA IGGUAUGG | 15742 |
| 1465 | UGGAGGAC U UCCAGGGA | 6403 | UCCCUGGA CUGAUGAGGCCGUUAGGCCGAA IUCCUCCA | 15743 |
| 1468 | AGGACUUC C AGGGAGGA | 6404 | UCCUCCCU CUGAUGAGGCCGUUAGGCCGAA IAAGUCCU | 15744 |
| 1469 | GGACUUCC A GGGAGGAA | 6405 | UUCCUCCC CUGAUGAGGCCGUUAGGCCGAA IGAAGUCC | 15745 |
| 1502 | UAAAAAUC A AUUUGCUC | 6406 | GAGCAAAU CUGAUGAGGCCGUUAGGCCGAA TAUUUUUA | 15746 |
| 1509 | CAAUUUGC U CUAAUUGA | 6407 | UCAAUUAG CUGAUGAGGCCGUUAGGCCGAA ICAAAUUG | 15747 |
| 1511 | AUUUGCUC U AAUUCAAG | 6408 | CUUCAAUU CUGAUGAGGCCGUUAGGCCGAA IAGCAAAU | 15748 |
| 1528 | GAAAAAAC A AAACUGUA | 6409 | UACAGUUU CUGAUGAGGCCGUUAGGCCGAA IUUUUUUC | 15749 |
| 1533 | AACAAAAC U CUAAGUAC | 6410 | GUACUUAC CUGAUGAGGCCGUUAGGCCGAA IUUUUGUU | 15750 |
| 1542 | GUAAGUAC C CUUGUUAU | 6411 | AUAACAAG CUGAUGAGGCCGUUAGGCCGAA IUACUUAC | 15751 |
| 1543 | UAAGUACC C UUGUUAUC | 6412 | GAUAACAA CUGAUGAGGCCGUUAGGCCGAA IGUACUUA | 15752 |
| 1544 | AAGUACCC U UGUUAUCC | 6413 | GGAUAACA CUGAUGAGGCCGUUAGGCCGAA ICCUACUU | 15753 |
| 1552 | UUGUUAUC C AAGCGGCA | 6414 | UGCCGCUU CUGAUGAGGCCGUUAGGCCGAA IAUAACAA | 15754 |
| 1553 | UGUUAUCC A AGCGGCAA | 6415 | UUGCCGCU CUGAUGAGGCCGUUAGGCCGAA IGAUAACA | 15755 |
| 1560 | CAAGCGGC A AAUGUGUC | 6416 | GACACAUU CUGAUGAGGCCGUUAGGCCGAA ICCGCUUG | 15756 |
| 1569 | AAUGUGUC A GCUUUGUA | 6417 | UACAAAGC CUGAUGAGGCCGUUAGGCCGAA IACACAUG | 15757 |
| 1572 | GUGUCAUC U UUGUACAA | 6418 | UUGUACAA CUGAUGAGGCCGUUAGGCCGAA ICUGACAC | 15758 |
| 1579 | CUGUGUAC A AAUGUGAA | 6419 | UUCACAUU CUGAUGAGGCCGUUAGGCCGAA IUACAAAG | 15759 |
| 1594 | AAGCGGUC A ACAAAGUC | 6420 | GACUUUGU CUGAUGAGGCCGUUAGGCCGAA IACCOCUG | 15760 |
| 1597 | CGGUCAAC A AAGUCGGG | 6421 | CCCGACUU CUGAUGAGGCCGUUAGGCCGAA IUUGACCG | 15761 |
| 1624 | GGGUGAUC U CCUUCCAC | 6422 | GUGGAAGG CUGAUGAGGCCGUUAGGCCGAA IAUCACCC | 15762 |
| 1626 | GUGAUCUC C UUCCACGU | 6423 | ACGUGGAA CUGAUGAGGCCGUUAGGCCGAA IAGAUCAC | 15763 |
| 1627 | UGAUCUCC U UCCACGUG | 6424 | CACCUGGA CUGAUGAGGCCGUUAGGCCGAA IGAGAUCA | 15764 |
| 1630 | UCUCCUUC C ACGUGACC | 6425 | GGUCACGU CUGAUGAGGCCGUUAGGCCGAA IAAGGAGA | 15765 |
| 1631 | CUCCUUCC A CGUGACCA | 6426 | UGGUCACG CUGAUGAGGCCGUUAGGCCGAA IGAAGGAG | 15766 |
| 1638 | CACGUGAC C AGGGGUCC | 6427 | GGACCCCU CUGAUGAGGCCGUUAGGCCGAA IUCACGUG | 15767 |
| 1639 | ACGUGACC A GGGGUCCU | 6428 | AGGACCCC CUGAUGAGGCCGUUAGGCCGAA IGUCACGU | 15768 |
| 1646 | CAGOGGUC C UGAAAUUA | 6429 | UAAUUUCA CUGAUGAGGCCGUUAGGCCGAA IACCCCUG | 15769 |
| 1647 | AGGGGUCC U GAAAUUAC | 6430 | GUAAUUUC CUGAUGAGGCCGUUAGGCCGAA IGACCCCU | 15770 |
| 1656 | GAAAUUAC U UUGCAACC | 6431 | GGUUGCAA CUGAUGAGGCCGUUAGGCCGAA IUAAUUUC | 15771 |
| 1661 | UACUUUGC A ACCUGACA | 6432 | UGUCAGGU CUGAUGAGGCCGUUAGGCCGAA ICAAAGUA | 15772 |
| 1664 | UUGCAAAC C UGACAUGC | 6433 | GCAUGUCA CUGAUGAGGCCGUUAGGCCGAA IUUGCAAA | 15773 |
| 1665 | UUGCAACC U GACAUGCA | 6434 | UGCAUGUC CUGAUGAGGCCGUUAGGCCGAA IGUUGCAA | 15774 |
| 1669 | AACCUGAC A UGCAGCCC | 6435 | GGGCUGCA CUGAUGAGGCCGUUAGGCCGAA IUCAGGUG | 15775 |
| 1673 | UGACAUGC A GCCCACUG | 6436 | CAGUGGGC CUGAUGAGGCCGUUAGGCCGAA ICAUGUCA | 15776 |
| 1676 | CAUGCAGC C CACUGAGC | 6437 | GCUCAGUG CUGAUGAGGCCGUUAGGCCGAA ICUCCAUG | 15777 |
| 1677 | AUGCAGCC C ACUGAGCA | 6438 | UGCUCAGU CUGAUGAGGCCGUUAGGCCGAA IGCUGCAU | 15778 |
| 1678 | UGCAGCCC A CUGAGCAG | 6439 | CUGCUCAG CUGAUGAGGCCGUUAGGCCGAA IGGCUGCA | 15779 |
| 1680 | CAGCCCAC U GAGCAGGA | 6440 | UCCUGCUC CUGAUGAGGCCGUUAGGCCGAA IUGGGCUG | 15780 |
| 1685 | CACUGAGC A GGAGAGCG | 6441 | CGCUCUCC CUGAUGAGGCCGUUAGGCCGAA ICUCAGUG | 15781 |
| 1698 | AGCGUGUC U UGGUGGUG | 6442 | CACCACAA CUGAUGAGGCCGUUAGGCCGAA IACACGCU | 15782 |
| 1708 | UGUGGUGC A CUGCAGAC | 6443 | GUCUOCAG CUGAUGAGGCCGUUAGGCCGAA ICACCACA | 15783 |
| 1710 | UGGUGCAC U GCAGACAG | 6444 | CUGUCUGC CUGAUGAGGCCGUUAGGCCGAA IUGCACCA | 15784 |
| 1713 | UGCACUGC A GACAGAUC | 6445 | GAUCUGUC CUGAUGAGGCCGUUAGGCCGAA ICAGUGCA | 15785 |
| 1717 | CUGCAGAC A GAUCUACG | 6446 | CGUAGAUC CUGAUGAGGCCGUUAGGCCGAA IUCUGCAG | 15786 |
| 1722 | GACAGAUC U ACGUUUGA | 6447 | UCAAACGU CUGAUGAGGCCGUUAGGCCGAA IAUCUGUC | 15787 |
| 1735 | UUGAGAAC C UCACAUGG | 6448 | CCAUGUGA CUGAUGAGGCCGUUAGGCCGAA IUUCUCAA | 15788 |
| 1736 | UGAGAACC U CACAUGGU | 6449 | ACCAUGUG CUGAUGAGGCCGUUAGGCCGAA IGUUCUCA | 15789 |
| 1738 | AGAACCUC A CAUGGUAC | 6450 | GUACCAUG CUGAUGAGGCCGUUAGGCCGAA IAGGUUCU | 15790 |
| 1740 | AACCUCAC A UGGUACAA | 6451 | UGGUACCA CUGAUGAGGCCGUUAGGCCGAA IUGAGGUU | 15791 |
| 1747 | CAUGGUAC A AGCUUGGC | 6452 | GCCAAGCU CUGAUGAGGCCGUUAGGCCGAA IGACCAUG | 15792 |
| 1751 | GUACAAGC U UGGCCCAC | 6453 | GUGGGCCA CUGAUGAGGCCGUUAGGCCGAA ICUUCUAC | 15793 |
| 1756 | AGCUUGGC C CACAGCCU | 6454 | AGGCUGUG CUGAUGAGGCCGUUAGGCCGAA ICCAAGCU | 15794 |
| 1757 | GCUUGGCC C ACAGCCUC | 6455 | GAGGCUGU CUGAUGAGGCCGUUAGGCCGAA IGCCAAGC | 15795 |
| 1758 | CUUGGCCC A CAGCCUCU | 6456 | AGAGGCUG CUGAUGAGGCCGUUAGGCCGAA IGGCCAAG | 15796 |
| 1760 | UGGCCCAC A GCCUCUGC | 6457 | GCAGAGCC CUGAUGAGGCCGUUAGGCCGAA IUGCGCCA | 15797 |
| 1763 | CCCACAGC C UCUGCCAA | 6458 | UUGCCAGA CUGAUGAGGCCGUUAGGCCGAA ICUGUGGG | 15798 |
| 1764 | CCACAGCC U CUGCCAAU | 6459 | AUUGGCAG CUGAUGAGGCCGUUAGGCCGAA ICCUGUGG | 15799 |
| 1766 | ACAGCCUC U GCCAAUCC | 6460 | GGAUUGGC CUGAUGAGGCCGUUAGGCCGAA IAGGCUGU | 15800 |
| 1769 | GCCUCUGC C AAUCCAUG | 6461 | CAUGGAUC CUGAUGAGGCCGUUAGGCCGAA ICAGAGUC | 15801 |
| 1770 | CCUCUGCC A AUCCAUGU | 6462 | ACAUGGAU CUGAUGAGGCCGUUAGGCCGAA IGCAGAGG | 15802 |
| 1774 | UGCCAAUC C AUGUGGGA | 6463 | UCCCACAU CUGAUGAGGCCGUUAGGCCGAA IAUUGGCA | 15803 |
| 1775 | GCCAAUCC A UGUGGGAG | 6464 | CUCCCACA CUGAUGAGGCCGUUAGGCCGAA IGAUUGGC | 15804 |
| 1790 | AGAGUUGC C CACACCUG | 6465 | CAGGUGUG CUGAUGAGGCCGUUAGGCCGAA ICAACUCU | 15805 |
| 1791 | GAGUUGCC C ACACCUGU | 6466 | ACAGGUGU CUGAUGAGGCCGUUAGGCCGAA IGCAACUC | 15806 |
| 1792 | AGUUGCCC A CACCUGUU | 6467 | AACAGGUG CUGAUGAGGCCGUUAGGCCGAA IGGCAACU | 15807 |
| 1794 | UUGCCCAC A CCUGUUUG | 6468 | CAAACAGG CUGAUGAGGCCGUUAGGCCGAA IUGGGCAA | 15808 |
| 1796 | GCCCACAC C UGUUUGCA | 6469 | UGCAAACA CUGAUGAGGCCGUUAGGCCGAA IUGUGGGC | 15809 |
| 1797 | CCCACACC U GUUUGCAA | 6470 | UUGCAAAC CUGAUGAGGCCGUUAGGCCGAA IGUGUGGG | 15810 |
| 1804 | CUGUUUGC A AGAACUUG | 6471 | CAAGUUCU CUGAUGAGGCCGUUAGGCCGAA ICAAACAG | 15811 |
| 1810 | GCAAGAAC U UGGAUACU | 6472 | AGUAUCCA CUGAUGAGGCCGUUAGGCCGAA IUUCUUGC | 15812 |
| 1818 | UUGGAUAC U CUUUGGAA | 6473 | UUCCAAAG CUGAUGAGGCCGUUAGGCCGAA IUAUCCAA | 15813 |
| 1820 | GGAUACUC U UUGGAAAU | 6474 | AUUCCCAA CUGAUGAGGCCGUUAGGCCGAA IAGUAUCC | 15814 |
| 1836 | UUGAAUGC C ACCAUGUU | 6475 | AACAUGGU CUGAUGAGGCCGUUAGGCCGAA ICAUUCAA | 15815 |
| 1837 | UGAAUGCC A CCAUGUUC | 6476 | GAACAUGG CUGAUGAGGCCGUUAGGCCGAA IGCAUUCA | 15816 |
| 1839 | AAUGCCAC C AUGUUCUC | 6477 | GAGAACAU CUGAUGAGGCCGUUAGGCCGAA IUGGCAUU | 15817 |
| 1840 | AUGCCACC A UGUUCUCU | 6478 | AGAGAACA CUGAUGAGGCCGUUAGGCCGAA IGUGGCAU | 15818 |
| 1846 | CCAUGUUC U CUAAUAGC | 6479 | GCUAUUAG CUGAUGAGGCCGUUAGGCCGAA IAACAUGG | 15819 |
| 1848 | AUGUUCUC U AAUAGCAC | 6480 | GUGCUAUU CUGAUGAGGCCGUUAGGCCGAA IAGAACAU | 15820 |
| 1855 | CUAAUAGC A CAAAUGAC | 6481 | GUCAUUUG CUGAUGAGGCCGUUAGGCCGAA ICUADUAG | 15821 |
| 1857 | AAUAGCAC A AAUGACAU | 6482 | AUGUCAUC CUGAUGAGGCCGUUAGGCCGAA IUGCUAUU | 15822 |
| 1864 | CAAAUGAC A UCCUGAUC | 6483 | GAUCAAAA CUGAUGAGGCCGUUAGGCCGAA IUCAUUUG | 15823 |
| 1873 | UUUUGAUC A UGGAGCUU | 6484 | AAGCUCCA CUGAUGAGGCCGUUAGGCCGAA IAUCAAAA | 15824 |
| 1880 | CAUGGAGC U UAAGAAUG | 6485 | CAUUCUUA CUGAUGAGGCCGUUAGGCCGAA ICUCCAUG | 15825 |
| 1890 | AAGAAUGC A UCCUUGCA | 6486 | UGCAAGGA CUGAUGAGGCCGUUAGGCCGAA ICAUUCUU | 15826 |
| 1893 | AAUGCAUC C UUGCAGGA | 6487 | UCCUGCAA CUGAUGAGGCCGUUAGGCCGAA IAUGCAUU | 15827 |
| 1894 | AUGCAUCC U UGCAGGAC | 6488 | GUCCUGCA CUGAUGAGGCCGUUAGGCCGAA IGAUGCAU | 15828 |
| 1898 | AUCCUUGC A GGACCAAG | 6489 | CUUGGUCC CUGAUGAGGCCGUUAGGCCGAA ICAAGGAU | 15829 |
| 1903 | UGCAGGAC C AAGGAGAC | 6490 | GUCUCCUU CUGAUGAGGCCGUUAGGCCGAA IUCCUGCA | 15830 |
| 1904 | GCAGGACC A AGGAGACU | 6491 | AGUCUCCU CUGAUGAGGCCGUUAGGCCGAA IGUCCUGC | 15831 |
| 1912 | AAGGAGAC U AUGUCUGC | 6492 | GCAGACAU CUGAUGAGGCCGUUAGGCCGAA IUCUCCUC | 15832 |
| 1918 | ACUAUGUC U GCCUUGCU | 6493 | AGCAAGGC CUGAUGAGGCCGUUAGGCCGAA IACAUAGU | 15833 |
| 1921 | AUGUCUGC C UUGCUCAA | 6494 | UUGAGCAA CUGAUGAGGCCGUUAGGCCGAA ICAGACAU | 15834 |
| 1922 | UGUCUGCC U UGCUCAAG | 6495 | CUUGAGCA CUGAUGAGGCCGUUAGGCCGAA IGCAGACA | 15835 |
| 1926 | UGCCUUGC U CAAGACAG | 6496 | CUGUCUUG CUGAUGAGGCCGUUAGGCCGAA ICAAGGCA | 15836 |
| 1928 | CCUUGCUC A AGACAGGA | 6497 | UCCUGUCU CUGAUGAGGCCGUUAGGCCGAA IAGCAAGG | 15837 |
| 1933 | CUCAAGAC A GGAAGACC | 6498 | GGUCUUCC CUGAUGAGGCCGUUAGGCCGAA IUCCUGAG | 15838 |
| 1941 | AGGAAGAC C AAGAAAAG | 6499 | CUUUUCUU CUGAUGAGGCCGUUAGGCCGAA IUCUUCCU | 15839 |
| 1942 | GGAAGACC A AGAAAAGA | 6500 | UCUUUUCU CUGAUGAGGCCGUUAGGCCGAA IGUCUUCC | 15840 |
| 1952 | GAAAAGAC A UUGCGUGG | 6501 | CCACGCAA CUGAUGAGGCCGUUAGGCCGAA IUCUUUUC | 15841 |
| 1963 | GCGUGGUC A GGCAGCUC | 6502 | GAGCUGCC CUGAUGAGGCCGUUAGGCCGAA IACCACGC | 15842 |
| 1967 | GGUCAGGC A GCUCACAG | 6503 | CUGUGAGC CUGAUGAGGCCGUUAGGCCGAA ICCUGACC | 15843 |
| 1970 | CAGGCAGC U CACAGUCC | 6504 | CGACUGUG CUGAUGAGGCCGUUAGGCCGAA ICUCCCUG | 15844 |
| 1972 | GGCAGCUC A CAGUCCUA | 6505 | UAGGACUG CUGAUGAGGCCGUUAGGCCGAA IAGCUGCC | 15845 |
| 1974 | CACCUCAC A GUCCUAGA | 6506 | UCUAGGAC CUGAUGAGGCCGUUAGGCCGAA IUGAGCUG | 15846 |
| 1978 | UCACAGUC C UAGAGCGU | 6507 | ACGCUCUA CUGAUGAGGCCGUUAGGCCGAA IACUGUGA | 15847 |
| 1979 | CACAGUCC U AGAUCCUG | 6508 | CACGCUCU CUGAUGAGGCCGUUAGGCCGAA IGACUGUG | 15848 |
| 1992 | CGUGUGGC A CCCACGAU | 6509 | AUCGUGGG CUGAUGAGGCCGUUAGGCCGAA ICCACACG | 15849 |
| 1994 | UGUGGCAC C CACGAUCA | 6510 | UGAUCGUG CUGAUGAGGCCGUUAGGCCGAA IUGCCACA | 15850 |
| 1995 | GUGGCACC C ACGAUCAC | 6511 | GUGAUCGU CUGAUGAGGCCGUUAGGCCGAA IGUGCCAC | 15851 |
| 1996 | UGGCACCC A CGAUCACA | 6512 | UGUGAUCG CUGAUGAGGCCGUUAGGCCGAA IGGUGCCA | 15852 |
| 2002 | CCACGAUC A CAGGAAAC | 6513 | GUUUCCUG CUGAUGAGGCCGUUAGGCCGAA IAUCGUGG | 15853 |
| 2004 | ACGAUCAC A GGAAACCU | 6514 | AGGUUUCC CUGAUGAGGCCGUUAGGCCGAA IUGAUCGU | 15854 |
| 2011 | CAGGAAAC C UGGAGAAU | 6515 | AUUCUCCA CUGAUGAGGCCGUUAGGCCGAA IUUUCCUG | 15855 |
| 2012 | AGGAAACC U GGAGAAUC | 6516 | GAUUCUCC CUGAUGAGGCCGUUAGGCCGAA IGUUUCCU | 15856 |
| 2021 | GGAGAAUC A GACGACAA | 6517 | UUGUCGUC CUGAUGAGGCCGUUAGGCCGAA IAUUCUCC | 15857 |
| 2028 | CAGACGAC A AGUAUUGG | 6518 | CCAAUACU CUGAUGAGGCCGUUAGGCCGAA IUCGUCUG | 15858 |
| 2044 | GGGAAAGC A UCGAAGUC | 6519 | GACUUCGA CUGAUGAGGCCGUUAGGCCGAA ICUUUCCC | 15859 |
| 2053 | UCGAAGUC U CAUGCACG | 6520 | CGUGCAUG CUGAUGAGGCCGUUAGGCCGAA IACUUCGA | 15860 |
| 2055 | GAAGUCUC A UGCACGGC | 6521 | GCCGUGCA CUGAUGAGGCCGUUAGGCCGAA IAGACUUC | 15861 |
| 2059 | UCUCAUGC A CGGCAUCU | 6522 | AGAUGCCG CUGAUGAGGCCGUUAGGCCGAA ICAUGAGA | 15862 |
| 2064 | UGCACGGC A UCUGGGAA | 6523 | UUCCCAGA CUGAUGAGGCCGUUAGGCCGAA ICCGUGCA | 15863 |
| 2067 | ACGGCAUC U GGGAAUCC | 6524 | GGAUUCCC CUGAUGAGGCCGUUAGGCCGAA IAUGCCGU | 15864 |
| 2075 | UGGGAAUC C CCCUCCAC | 6525 | GUGGAGGG CUGAUGAGGCCGUUAGGCCGAA IAUUCCCA | 15865 |
| 2076 | GGGAAUCC C CCUCCACA | 6526 | UGUGGAGG CUGAUGAGGCCGUUAGGCCGAA IGAUUCCC | 15866 |
| 2077 | GGAAUCCC C CUCCACAG | 6527 | CUGUGGAG CUGAUGAGGCCGUUAGGCCGAA IGGAUUCC | 15867 |
| 2078 | GAAUCCCC C UCCACAGA | 6528 | UCUGUGGA CUGAUGAGGCCGUUAGGCCGAA IGGGAUUC | 15868 |
| 2079 | AAUCCCCC U CCACAGAU | 6529 | AUCUGUGG CUGAUGAGGCCGUUAGGCCGAA IGGGGAUU | 15869 |
| 2081 | UCCCCCUC C ACAGAUCA | 6530 | UGAUCUGU CUGAUGAGGCCGUUAGGCCGAA IAGGGGGA | 15870 |
| 2082 | CCCCCUCC A CAGAUCAU | 6531 | AUGAUCUG CUGAUGAGGCCGUUAGGCCGAA IGAGGGGG | 15871 |
| 2084 | CCCUCCAC A GAUCAUGU | 6532 | ACAUGAUC CUGAUGAGGCCGUUAGGCCGAA IUGGAGGG | 15872 |
| 2089 | CACAGAUC A UGUGGUUU | 6533 | AAACCACA CUGAUGAGGCCGUUAGGCCGAA IAUCUGUG | 15873 |
| 2112 | AAUGAGAC C CUUGUAGA | 6534 | UCUACAAG CUGAUGAGGCCGUUAGGCCGAA IUCUCAUU | 15874 |
| 2113 | AUGAGACC C UUGUAGAA | 6535 | UUCUACAA CUGAUGAGGCCGUUAGGCCGAA IGUCUCAU | 15875 |
| 2114 | UGAGACCC U UGUAGAAG | 6536 | CUUCUACA CUGAUGAGGCCGUUAGGCCGAA IGGUCUCA | 15876 |
| 2125 | UAGAAGAC U CAGGCAUU | 6537 | AAUGCCUG CUGAUGAGGCCGUUAGGCCGAA IUCUUCUA | 15877 |
| 2127 | GAAGACUC A GGCAUUGU | 6538 | ACAAUGCC CUGAUGAGGCCGUUAGGCCGAA IAGUCUUC | 15878 |
| 2131 | ACUCAGGC A UUGUAUUG | 6539 | CAAUACAA CUGAUGAGGCCGUUAGGCCGAA ICCUGAGU | 15879 |
| 2152 | AUGGGAAC C GGAACCUC | 6540 | GAGGUUCC CUGAUGAGGCCGUUAGGCCGAA IUUCCCAU | 15880 |
| 2158 | ACCGGAAC C UCACUAUC | 6541 | GAUAGUGA CUGAUGAGGCCGUUAGGCCGAA IUUCCGGU | 15881 |
| 2159 | CCGGAACC U CACUAUCC | 6542 | GGAUAGUG CUGAUGAGGCCGUUAGGCCGAA IGUUCCGG | 15882 |
| 2161 | GGAACCUC A CUAUCCGC | 6543 | GCGGAUAG CUGAUGAGGCCGUUAGGCCGAA IAGGUUCC | 15883 |
| 2163 | AACCUCAC U AUCCGCAG | 6544 | CUGCGGAU CUGAUGAGGCCGUUAGGCCGAA IUGAGGUU | 15884 |
| 2167 | UCACUAUC C GCAGAGUG | 6545 | CACUCUGC CUGAUGAGGCCGUUAGGCCGAA IAUAGUGA | 15885 |
| 2170 | CUAUCCGC A GAGUGAGG | 6546 | CCUCACUC CUGAUGAGGCCGUUAGGCCGAA ICGGAUAG | 15886 |
| 2194 | ACGAAGGC C UCUACACC | 6547 | GGUGUAGA CUGAUGAGGCCGUUAGGCCGAA ICCUUCGU | 15887 |
| 2195 | CGAAGGCC U CUACACCU | 6548 | AGGUGUAG CUGAUGAGGCCGUUAGGCCGAA IGCCUUCG | 15888 |
| 2197 | AAGGCCUC U ACACCUGC | 6549 | GCAGGUGU CUGAUGAGGCCGUUAGGCCGAA IAGGCCUU | 15889 |
| 2200 | GCCUCUAC A CCUGCCAG | 6550 | CUGGCAGG CUGAUGAGGCCGUUAGGCCGAA IUAGAGGC | 15890 |
| 2202 | CUCUACAC C UGCCAGGC | 6551 | GCCUGGCA CUGAUGAGGCCGUUAGGCCGAA IUGUAGAG | 15891 |
| 2203 | UCUACACC U GCCAGGCA | 6552 | UGCCUGGC CUGAUGAGGCCGUUAGGCCGAA IGUGUAGA | 15892 |
| 2206 | ACACCUGC C AGGCAUGC | 6553 | GCAUGCCU CUGAUGAGGCCGUUAGGCCGAA ICAGGUGU | 15893 |
| 2207 | CACCUGCC A GGCAUGCA | 6554 | UGCAUGCC CUGAUGAGGCCGUUAGGCCGAA IGCAGGUG | 15894 |
| 2211 | UGCCAGGC A UGCAGUGU | 6555 | ACACUGCA CUGAUGAGGCCGUUAGGCCGAA ICCUGGCA | 15895 |
| 2215 | AGGCAUGC A GUGUUCUU | 6556 | AAGAACAC CUGAUGAGGCCGUUAGGCCGAA ICAUGCCU | 15896 |
| 2222 | CAGUGUUC U UGGCUGUG | 6557 | CACAGCCA CUGAUGAGGCCGUUAGGCCGAA IAACACUG | 15897 |
| 2227 | UUCUUGGC U GUGCAAAA | 6558 | UUUUGCAC CUGAUGAGGCCGUUAGGCCGAA ICCAAGAA | 15898 |
| 2232 | GGCUGUGC A AAAGUGGA | 6559 | UCCACUUU CUGAUGAGGCCGUUAGGCCGAA ICACAGCC | 15899 |
| 2244 | GUGGAGOC A UUUUUCAU | 6560 | AUGAAAAA CUGAUGAGGCCGUUAGGCCGAA ICCUCCAC | 15900 |
| 2251 | CAUUUUUC A UAAUAGAA | 6561 | UUCUAUUA CUGAUGAGGCCGUUAGGCCGAA IAAAAAUG | 15901 |
| 2265 | GAAGGUGC C CAGGAAAA | 6562 | UUUUCCUG CUGAUGAGGCCGUUAGGCCGAA ICACCUUC | 15902 |
| 2266 | AAGGUGCC C AGGAAAAG | 6563 | CUUUUCCU CUGAUGAGGCCGUUAGGCCGAA IGCACCUU | 15903 |
| 2267 | AGGUGCCC A GGAAAAGA | 6564 | UCUUUUCC CUGAUGAGGCCGUUAGGCCGAA IGGCACCU | 15904 |
| 2281 | AGACGAAC U UGGAAAUC | 6565 | GAUUUCCA CUGAUGAGGCCGUUAGGCCGAA IUUCGUCU | 15905 |
| 2290 | UGGAAAUC A UUAUUCUA | 6566 | UAGAAUAA CUGAUGAGGCCGUUAGGCCGAA IAGUUCCA | 15906 |
| 2297 | CAUUAUUC U AGUAGGCA | 6567 | UGCCUACU CUGAUGAGGCCGUUAGGCCGAA IAAUAAUG | 15907 |
| 2305 | UAGUAGGC A CGACGGUG | 6568 | CACCGUCG CUGAUGAGGCCGUUAGGCCGAA ICCUACUA | 15908 |
| 2319 | GUGAUUGC C AUGUUCUU | 6569 | AAGAACAU CUGAUGAGGCCGUUAGGCCGAA ICAAUCAC | 15909 |
| 2320 | UGAUUGCC A UGUUCUUC | 6570 | GAAGAACA CUGAUGAGGCCGUUAGGCCGAA IGCAAUCA | 15910 |
| 2326 | CCAUGUUC U UCUGGCUA | 6571 | UAGCCAGA CUGAUGAGGCCGUUAGGCCGAA IAACAUGG | 15911 |
| 2329 | UGUUCUUC U GGCUACUU | 6572 | AAGUAGCC CUGAUGAGGCCGUUAGGCCGAA IAAGAACA | 15912 |
| 2333 | CUUCUGGC U ACUUCUUG | 6573 | CAAGAAGU CUGAUGAGGCCGUUAGGCCGAA ICCAGAAG | 15913 |
| 2336 | CUGGCUAC U UCUUGUCA | 6574 | UGACAAGA CUGAUGAGGCCGUUAGGCCGAA IUAGCCAG | 15914 |
| 2339 | GCUACUUC U UGUCAUCA | 6575 | UGAUGACA CUGAUGAGGCCGUUAGGCCGAA IAAGUAGC | 15915 |
| 2344 | UUCUUGUC A UCAUCCUA | 6576 | UAGGAUGA CUGAUGAGGCCGUUAGGCCGAA IACAAGAA | 15916 |
| 2347 | UUGUCAUC A UCCUAGGG | 6577 | CCCUAGGA CUGAUGAGGCCGUUAGGCCGAA IAUGACAA | 15917 |
| 2350 | UCAUCAUC C UAGGGACC | 6578 | GGUCCCUA CUGAUGAGGCCGUUAGGCCGAA IAUGAUGA | 15918 |
| 2351 | CAUCAUCC U AGGGACCG | 6579 | CGGUCCCU CUGAUGAGGCCGUUAGGCCGAA IGAUGAUG | 15919 |
| 2358 | CUAGGGAC C GUUAAGCG | 6580 | CGCUUAAC CUGAUGAGGCCGUUAGGCCGAA IUCCCUAG | 15920 |
| 2370 | AUGCGGGC C AAUGGAGG | 6581 | CCUCCAUU CUGAUGAGGCCGUUAGGCCGAA ICCCGCUU | 15921 |
| 2371 | AGCGGGCC A AUGGAGOG | 6582 | CCCUCCAU CUGAUGAGGCCGUUAGGCCGAA IGCCCGCU | 15922 |
| 2384 | AGGGGAAC U GAAGACAG | 6583 | CUGUCUUC CUGAUGAGGCCGUUAGGCCGAA IUUCCCCU | 15923 |
| 2391 | CUGAAGAC A GGCUACUU | 6584 | AAGUAGCC CUGAUGAGGCCGUUAGGCCGAA IUCUUCAG | 15924 |
| 2395 | AGACAGGC U ACUUGUCC | 6585 | GGACAAGU CUGAUGAGGCCGUUAGGCCGAA ICCUGUCU | 15925 |
| 2398 | CAGGCUAC U UGUCCAUC | 6586 | GAUGGACA CUGAUGAGGCCGUUAGGCCGAA IUAGCCUG | 15926 |
| 2403 | UACUUGUC C AUCGUCAU | 6587 | AUGACGAU CUGAUGAGGCCGUUAGGCCGAA IACAAGUA | 15927 |
| 2404 | ACUUGUCC A UCCUCAUG | 6588 | CAUGACGA CUGAUGAGGCCGUUAGGCCGAA IGACAAGU | 15928 |
| 2410 | CCAUCGUC A UGGAUCCA | 6589 | UGGAUCCA CUGAUGAGGCCGUUAGGCCGAA IACGAUGG | 15929 |
| 2417 | CAUGGAUC C AGAUGAAC | 6590 | GUUCAUCU CUGAUGAGGCCGUUAGGCCGAA IAUCCAUG | 15930 |
| 2418 | AUGGAUCC A GAUGAACU | 6591 | AGUUCAUC CUGAUGAGGCCGUUAGGCCGAA IGAUCCAU | 15931 |
| 2426 | AGAUGAAC U CCCAUUGG | 6592 | CCAAUGGG CUGAUGAGGCCGUUAGGCCGAA IUUCAUCU | 15932 |
| 2428 | AUGAACUC C CAUUGGAU | 6593 | AUCCAAUG CUGAUGAGGCCGUUAGGCCGAA IAGUUCAU | 15933 |
| 2429 | UGAACUCC C AUUGGAUG | 6594 | CAUCCAAU CUGAUGAGGCCGUUAGGCCGAA IGAGUUCA | 15934 |
| 2430 | GAACUCCC A UUGGAUGA | 6595 | UCAUCCAA CUGAUGAGGCCGUUAGGCCGAA IGGAGUUC | 15935 |
| 2441 | GGAUGAAC A UUGUGAAC | 6596 | GUUCACAA CUGAUGAGGCCGUUAGGCCGAA IUUCAUCC | 15936 |
| 2453 | UGAACGAC U GCCUUAUG | 6597 | CAUAAGGC CUGAUGAGGCCGUUAGGCCGAA IUCGUUCA | 15937 |
| 2456 | ACGACUGC C UUAUGAUG | 6598 | CAUCAUAA CUGAUGAGGCCGUUAGGCCGAA ICAGUCGU | 15938 |
| 2457 | CGACUGCC U UAUGAUGC | 6599 | GCAUCAUA CUGAUGAGGCCGUUAGGCCGAA IGCAGUCG | 15939 |
| 2466 | UAUGAUGC C AGCAAAUG | 6600 | CAUUUGCU CUGAUGAGGCCGUUAGGCCGAA ICAUCAUA | 15940 |
| 2467 | AUGAUGCC A GCAAAUGG | 6601 | CCAUUUGC CUGAUGAGGCCGUUAGGCCGAA IGCAUCAU | 15941 |
| 2470 | AUGCCAGC A AAUGGGAA | 6602 | UUCCCAUU CUGAUGAGGCCGUUAGGCCGAA ICUGGCAU | 15942 |
| 2482 | GGGAAUUC C CCAGAGAC | 6603 | GUCUCUGG CUGAUGAGGCCGUUAGGCCGAA IAAUUCCC | 15943 |
| 2483 | GGAAUUCC C CAGAGACC | 6604 | GGUCUCUG CUGAUGAGGCCGUUAGGCCGAA IGAAUUCC | 15944 |
| 2484 | GAAUUCCC C AGAGACCG | 6605 | CGGUCUCU CUGAUGAGGCCGUUAGGCCGAA IGGAAUUC | 15945 |
| 2485 | AAUUCCCC A GAGACCGG | 6606 | CCGGUCUC CUGAUGAGGCCGUUAGGCCGAA IGGGAAUU | 15946 |
| 2491 | CCAGAGAC C GGCUGAAC | 6607 | GUUCAGCC CUGAUGAGGCCGUUAGGCCGAA IUCUCUGG | 15947 |
| 2495 | AGACCGGC U GAACCUAG | 6608 | CUAGGUUC CUGAUGAGGCCGUUAGGCCGAA ICCGGUCU | 15948 |
| 2500 | GGCUGAAC C UAGGUAAG | 6609 | CUUACCUA CUGAUGAGGCCGUUAGGCCGAA IUUCAGCC | 15949 |
| 2501 | GCUGAACC U AGGUAAGC | 6610 | GCUUACCU CUGAUGAGGCCGUUAGGCCGAA IGUUCACC | 15950 |
| 2510 | AGUUAAGC C UCUUUGCC | 6611 | GGCCAAGA CUGAUGAGGCCGUUAGGCCGAA ICUUACCU | 15951 |
| 2511 | GGUAAGCC U CUUGGCCG | 6612 | CGGCCAAG CUGAUGAGGCCGUUAGGCCGAA IGCUUACC | 15952 |
| 2513 | UAAGCCUC U UGGCCGUG | 6613 | CACCGCCA CUGAUGAGGCCGUUAGGCCGAA IACGCUUA | 15953 |
| 2518 | CUCUUGCC C GUGGUGCC | 6614 | GGCACCAC CUGAUGAGGCCGUUAGGCCGAA ICCAAGAG | 15954 |
| 2526 | CGUGGUUC C UUUGGCCA | 6615 | UGGCCAAA CUGAUGAGGCCGUUAGGCCGAA ICACCACG | 15955 |
| 2527 | GUGGUGCC U UUGGCCAA | 6616 | UUGGCCAA CUGAUGAGGCCGUUAGGCCGAA IGCACCAC | 15956 |
| 2533 | CCUUUGGC C AAGAGAUU | 6617 | AAUCUCUU CUGAUGAGGCCGUUAGGCCGAA ICCAAAGG | 15957 |
| 2534 | CUUUGGCC A AGAGAUUC | 6618 | CAAUCUCU CUGAUGAGGCCGUUAGGCCGAA IGCCAAAG | 15958 |
| 2547 | AUUGAAGC A GAUGCCUU | 6619 | AAGGCAUC CUGAUGAGGCCGUUAGGCCGAA ICUUCAAU | 15959 |
| 2553 | GCAGAUGC C UUUGGAAU | 6620 | AUUCCAAA CUGAUGAGGCCGUUAGGCCGAA ICAUCUGC | 15960 |
| 2554 | CAGAUGCC U UUGGAAUU | 6621 | AAUUCCAA CUGAUGAGGCCGUUAGGCCGAA IUCAUCUG | 15961 |
| 2566 | GAAUUGAC A AGACAGCA | 6622 | UGCUGUCU CUGAUGAGGCCGUUAGGCCGAA IUCAAUUC | 15962 |
| 2571 | GACAAGAC A GCAACUUG | 6623 | CAAGUUGC CUGAUGAGGCCGUUAGGCCGAA IUCUGGUC | 15963 |
| 2574 | AAGACAGC A ACUGUCAG | 6624 | CUGCAAGU CUGAUGAGGCCGUUAGGCCGAA ICUGUCUU | 15964 |
| 2577 | ACAGCAAC U UGCAGGAC | 6625 | GUCCUGCA CUGAUGAGGCCGUUAGGCCGAA IUUGCUGU | 15965 |
| 2581 | CAACUUGC A GGACAGUA | 6626 | UACUGUCC CUGAUGAGGCCGUUAGGCCGAA ICAAGUUG | 15966 |
| 2586 | UCCAGGAC A GUAGCAGU | 6627 | ACUGCUAC CUGAUGAGGCCGUUAGGCCGAA IUCCUGCA | 15967 |
| 2592 | ACAGUAGC A GUCAAAAU | 6628 | AUUUUGAC CUGAUGAGGCCGUUAGGCCGAA ICUACUGU | 15968 |
| 2596 | UAGCAGUC A AAAUGUUG | 6629 | CAACAUUU CUGAUGAGGCCGUUAGGCCGAA IACUGCUA | 15969 |
| 2616 | GAAGGAGC A ACACACAG | 6630 | CUGUGUGU CUGAUGAGGCCGUUAGGCCGAA ICUCCUUC | 15970 |
| 2619 | GGAGCAAC A CACAGUGA | 6631 | UCACUGUG CUGAUGAGGCCGUUAGGCCGAA IUUGCUCC | 15971 |
| 2621 | AGCAACAC A CAGUGAGC | 6632 | GCUCACUG CUGAUGAGGCCGUUAGGCCGAA IUGUUGCU | 15972 |
| 2623 | CAACACAC A GUGAGCAU | 6633 | AUGCUCAC CUGAUGAGGCCGUUAGGCCGAA IUGUGUUG | 15973 |
| 2630 | CAGUGAGC A UCGAGCUC | 6634 | GAGCUCGA CUGAUGAGGCCGUUAGGCCGAA ICUCACUG | 15974 |
| 2637 | CAUCGAGC U CUCAUGUC | 6635 | GACAUGAG CUGAUGAGGCCGUUAGGCCGAA ICUCGAUG | 15975 |
| 2639 | UCGAGCUC U CAUGUCUG | 6636 | CAGACAUG CUGAUGAGGCCGUUAGGCCGAA IAGCUCGA | 15976 |
| 2641 | GAGCUCUC A UGUCUGAA | 6637 | UUCAGACA CUGAUGAGGCCGUUAGGCCGAA IAGAGCUC | 15977 |
| 2646 | CUCAUGUC U GAACUCAA | 6638 | UUGAGUUC CUGAUGAGGCCGUUAGGCCGAA IACAUGAG | 15978 |
| 2651 | GUCUGAAC U CAAGAUCC | 6639 | GGAUCUUG CUGAUGAGGCCGUUAGGCCGAA IUUCAGAC | 15979 |
| 2653 | CUGAACUC A AGAUCCUC | 6640 | GAGGAUCU CUGAUGAGGCCGUUAGGCCGAA IAGUUCAG | 15980 |
| 2659 | UCAAGAUC C UCAUUCAU | 6641 | AUGAAUGA CUGAUGAGGCCGUUAGGCCGAA IAUCUUGA | 15981 |
| 2660 | CAAGAUCC U CAUUCAUA | 6642 | UAUGAAUG CUGAUGAGGCCGUUAGGCCGAA IGAUCUUG | 15982 |
| 2662 | AGAUCCUC A UUCAUAUU | 6643 | AAUAUGAA CUGAUGAGGCCGUUAGGCCGAA IAGGAUCU | 15983 |
| 2666 | CCUCAUUC A UAUUGGUC | 6644 | GACCAAUA CUGAUGAGGCCGUUAGGCCGAA IAAUGAGG | 15984 |
| 2675 | UAUUGGUC A CCAUCUCA | 6645 | UGAGAUGG CUGAUGAGGCCGUUAGGCCGAA IACCAAUA | 15985 |
| 2677 | UUGGUCAC C AUCUCAAU | 6646 | AUUGAGAU CUGAUGAGGCCGUUAGGCCGAA IUGACCAA | 15986 |
| 2678 | UGGUCACC A UCUCAAUG | 6647 | CAUGGAGA CUGAUGAGGCCGUUAGGCCGAA IGUGACCA | 15987 |
| 2681 | UCACCAUC U CAAUGUGG | 6648 | CCACAUUG CUGAUGAGGCCGUUAGGCCGAA IAUGGUGA | 15988 |
| 2683 | ACCAUCUC A AUCUGGUC | 6649 | GACCACAU CUGAUGAGGCCGUUAGGCCGAA IAGAUGGU | 15989 |
| 2692 | AUGUGGUC A ACCUUCUA | 6650 | UAGAAGGU CUGAUGAGGCCGUUAGGCCGAA IACCACAU | 15990 |
| 2695 | UGGUCAAC C UUCUAGGU | 6651 | ACCUAGAA CUGAUGAGGCCGUUAGGCCGAA IUUGACCA | 15991 |
| 2696 | GGUCAACC U UCUAGGUG | 6652 | CACCUAGA CUGAUGAGGCCGUUAGGCCGAA IGUUGACC | 15992 |
| 2699 | CAACCUUC U AGGUGCCU | 6653 | AGGCACCU CUGAUGAGGCCGUUAGGCCGAA IAAGGUUG | 15993 |
| 2706 | CUAGGUGC C UGUACCAA | 6654 | UHUGUACA CUGAUGAGGCCGUUAGGCCGAA ICACCUAG | 15994 |
| 2707 | UAGGUGCC U GUACCAAG | 6655 | CUUGGUAC CUGAUGAGGCCGUUAGGCCGAA IGCACCUA | 15995 |
| 2712 | GCCUGUAC C AAGCCAGG | 6656 | CCUGGCUU CUGAUGAGGCCGUUAGGCCGAA IUACAGGC | 15996 |
| 2713 | CCUGUACC A AGCCAGGA | 6657 | UCCUGGCU CUGAUGAGGCCGUUAGGCCGAA IGUACAGG | 15997 |
| 2717 | UACCAAGC C AGGAGGGC | 6658 | GCCCUCCU CUGAUGAGGCCGUUAGGCCGAA ICUUGGUA | 15998 |
| 2718 | ACCAAGCC A GGAGGGCC | 6659 | GGCCCUCC CUGAUGAGGCCGUUAGGCCGAA IGCUUGGU | 15999 |
| 2726 | AGGAGGGC C ACUCAUGG | 6660 | CCAUGAGU CUGAUGAGGCCGUUAGGCCGAA ICCCUCCU | 16000 |
| 2727 | GGAGGGCC A CUCAUGGU | 6661 | ACCAUGAG CUGAUGAGGCCGUUAGGCCGAA IGCCCUCC | 16001 |
| 2729 | AGGGCCAC U CAUGGUGA | 6662 | UCACCAUG CUGAUGAGGCCGUUAGGCCGAA IUGGCCCU | 16002 |
| 2731 | GGCCACUC A UGGUGAUU | 6663 | AAUCACCA CUGAUGAGGCCGUUAGGCCGAA IAGUGGCC | 16003 |
| 2749 | UGGAAUUC U GCAAAUUU | 6664 | AAAUUUGC CUGAUGAGGCCGUUAGGCCGAA IAAUUCCA | 16004 |
| 2752 | AAUUCUGC A AAUUUGGA | 6665 | UCCAAAUU CUGAUGAGGCCGUUAGGCCGAA ICAGAAUU | 16005 |
| 2764 | UUGGAAAC C UGUCCACU | 6666 | AGUGGACA CUGAUGAGGCCGUUAGGCCGAA IUUUCCAA | 16006 |
| 2765 | UGGAAACC U GUCCACUU | 6667 | AAGUGGAC CUGAUGAGGCCGUUAGGCCGAA IGUUUCCA | 16007 |
| 2769 | AACCUGUC C ACUUACCU | 6668 | AGGUAAGU CUGAUGAGGCCGUUAGGCCGAA IACAGGUU | 16008 |
| 2770 | ACCUGUCC A CUUACCUG | 6669 | CAGGUAAG CUGAUGAGGCCGUUAGGCCGAA IGACAGGU | 16009 |
| 2772 | CUGUCCAC U HACCUGAG | 6670 | CUCAGGUA CUGAUGAGGCCGUUAGGCCGAA IUGGACAG | 16010 |
| 2776 | CCACUUAC C UGAGGAGC | 6671 | GCUCCUCA CUGAUGAGGCCGUUAGGCCGAA IUAAGUGG | 16011 |
| 2777 | CACUUACC U GAGGACCA | 6672 | UCCUCCUC CUGAUGAGGCCGUUAGGCCGAA IGUAAGUG | 16012 |
| 2785 | UGAGGAGC A AGAGAAAU | 6673 | AUUUCUCU CUGAUGAGGCCGUUAGGCCGAA ICUCCUCA | 16013 |
| 2803 | AAUUUGUC C CCUACAAG | 6674 | CUUGUAGG CUGAUGAGGCCGUUAGGCCGAA IACAAAUU | 16014 |
| 2804 | AUUUGUCC C CUACAAGA | 6675 | UCUUGUAG CUGAUGAGGCCGUUAGGCCGAA IGACAAAU | 16015 |
| 2805 | UUUGUCCC C UACAAGAC | 6676 | GUCUUGUA CUGAUGAGGCCGUUAGGCCGAA IGGACAAA | 16016 |
| 2806 | UUGUCCCC U ACAAGACC | 6677 | GGUCUUGU CUGAUGAGGCCGUUAGGCCGAA IGGGACAA | 16017 |
| 2809 | UCCCCUAC A AGACCAAA | 6678 | UUUGGUCU CUGAUGAGGCCGUUAGGCCGAA IUAGGGGA | 16018 |
| 2814 | UACAAGAC C AAAGGGGC | 6679 | GCCCCUUU CUGAUGAGGCCGUUAGGCCGAA IUCUUGUA | 16019 |
| 2815 | ACAAGACC A AAGGGGCA | 6680 | UGCCCCUU CUGAUGAGGCCGUUAGGCCGAA IGUCUUGU | 16020 |
| 2823 | AAAGGGGC A CGAUUCCG | 6681 | CGGAAUCG CUGAUGAGGCCGUUAGGCCGAA ICCCCUUU | 16021 |
| 2830 | CACGAUUC C GUCAAGGG | 6682 | CCCUUGAC CUGAUGAGGCCGUUAGGCCGAA IAAUCGUG | 16022 |
| 2834 | AUHCCGUC A AGGGAAAG | 6683 | CUUUCCCU CUGAUGAGGCCGUUAGGCCGAA IACGGAAU | 16023 |
| 2845 | GGAAAGAC U ACGUUGGA | 6684 | UCCAACGU CUGAUGAGGCCGUUAGGCCGAA IUCUUUCC | 16024 |
| 2856 | GUUGGAGC A AUCCCUGU | 6685 | ACAGGGAU CUGAUGAGGCCGUUAGGCCGAA ICUCCAAC | 16025 |
| 2860 | GAGCAAUC C CUGUGGAU | 6686 | AUCCACAG CUGAUGAGGCCGUUAGGCCGAA IAUUGCUC | 16026 |
| 2861 | AGCAAUCC C UGUGGAUC | 6687 | GAUCCACA CUGAUGAGGCCGUUAGGCCGAA IGAUUGCU | 16027 |
| 2862 | GCAAUCCC U GUGGAUCU | 6688 | AGAUCCAC CUGAUGAGGCCGUUAGGCCGAA IGGAUUGC | 16028 |
| 2870 | UGUGGAUC U GAAACGGC | 6689 | GCCGUUUC CUGAUGAGGCCGUUAGGCCGAA IAUCCACA | 16029 |
| 2881 | AACGGCGC U UGGACAGC | 6690 | GCUGUCCA CUGAUGAGGCCGUUAGGCCGAA ICGCCGUU | 16030 |
| 2887 | GCUUGGAC A GCAUCACC | 6691 | GGUGAUGC CUGAUGAGGCCGUUAGGCCGAA IUCCAAGC | 16031 |
| 2890 | UGGACAGC A UCACCAGU | 6692 | ACUGGUGA CUGAUGAGGCCGUUAGGCCGAA ICUGUCCA | 16032 |
| 2893 | ACACCAUC A CCAGUAGC | 6693 | GCUACUGG CUGAUGAGGCCGUUAGGCCGAA IAUGCUGU | 16033 |
| 2895 | ACCAUCAC C AGUAGCCA | 6694 | UGGCUACU CUGAUGAGGCCGUUAGGCCGAA IUGAUGCU | 16034 |
| 2896 | GCAUCACC A GUAGCCAG | 6695 | CUGGCUAC CUGAUGAGGCCGUUAGGCCGAA IGUGAUGC | 16035 |
| 2902 | CCAGUAGC C AGACCUCA | 6696 | UGAGCUCU CUGAUGAGGCCGUUAGGCCGAA ICUACUGG | 16036 |
| 2903 | CAGUAGCC A GACCUCAG | 6697 | CUGACCUC CUGAUGAGGCCGUUAGGCCGAA ICCUACUG | 16037 |
| 2908 | GCCAGAGC U CAGCCAGC | 6698 | GCUGGCUG CUGAUGAGGCCGUUAGGCCGAA ICUCUGUC | 16038 |
| 2910 | CAGAGCUC A GCCAGCUC | 6699 | GAGCUGGC CUGAUGAGGCCGUUAGGCCGAA IAGCUCUG | 16039 |
| 2913 | ACCUCAUC C AGCUCUGG | 6700 | CCAGAGCU CUGAUGAGGCCGUUAGGCCGAA ICUGAGCU | 16040 |
| 2914 | GCUCAGCC A UCUCUGGA | 6701 | UCCAGAGC CUGAUGAGGCCGUUAGGCCGAA IGCUGAGC | 16041 |
| 2917 | CAGCCAGC U CUGGAUUU | 6702 | AAAUCCAG CUGAUGAGGCCGUUAGGCCGAA ICUGGCUG | 16042 |
| 2919 | GCCAUCUC U GGAUUUGU | 6703 | ACAAAUCC CUGAUGAGGCCGUUAGGCCGAA IAGCUGGC | 16043 |
| 2940 | GAGAAGUC C CUCAGUGA | 6704 | UCACUGAG CUGAUGAGGCCGUUAGGCCGAA IACUUCUC | 16044 |
| 2941 | AGAAGUCC C UCAGUGAU | 6705 | AUCACUGA CUGAUGAGGCCGUUAGGCCGAA IGACUUCU | 16045 |
| 2942 | GAAGUCCC U CAGUGAUG | 6706 | CAUCACUG CUGAUGAGGCCGUUAGGCCGAA IGGACCUC | 16046 |
| 2944 | AGUCCCUC A GUGAUGUA | 6707 | UACAUCAC CUGAUGAGGCCGUUAGGCCGAA IAGGGACU | 16047 |
| 2967 | GAGGAAGC U CCUGAAGA | 6708 | UCUUCAGG CUGAUGAGGCCGUUAGGCCGAA ICUUCCUC | 16048 |
| 2969 | GGAAGCUC C UGAAGAUC | 6709 | GAUCUUCA CUGAUGAGGCCGUUAGGCCGAA IAGCUUCC | 16049 |
| 2970 | GAAGCUCC U GAAGAUCU | 6710 | AGAUCUUC CUGAUGAGGCCGUUAGGCCGAA IGAGCUUC | 16050 |
| 2978 | UGAAGAUC U GUAUAAGG | 6711 | CCUUAUAC CUGAUGAGGCCGUUAGGCCGAA IAUCUUCA | 16051 |
| 2989 | AUAAGGAC U UCCUGACC | 6712 | GGUCAGGA CUGAUGAGGCCGUUAGGCCGAA IUCCUUAU | 16052 |
| 2992 | AGGACUUC C UGACCUUG | 6713 | CAAGGUCA CUGAUGAGGCCGUUAGGCCGAA IAAGUCCU | 16053 |
| 2993 | GGACUUCC U GACCUUGG | 6714 | CCAAGGUC CUGAUGAGGCCGUUAGGCCGAA IGAAGUCC | 16054 |
| 2997 | UUCCUGAC C UUGGAGCA | 6715 | UGCUCCAA CUGAUGAGGCCGUUAGGCCGAA IUCAGGAA | 16055 |
| 2998 | UCCUGACC U UGGAGCAU | 6716 | AUGCUCCA CUGAUGAGGCCGUUAGGCCGAA IGUCAGGA | 16056 |
| 3005 | CUUGGAGC A UCUCAUCU | 6717 | AGAUGAGA CUGAUGAGGCCGUUAGGCCGAA ICUCCAAG | 16057 |
| 3008 | GGAGCAUC U CAUCUGUU | 6718 | AACAGAUG CUGAUGAGGCCGUUAGGCCGAA IAUGCUCC | 16058 |
| 3010 | AGCAUCUC A UCUGUUAC | 6719 | GUAACAGA CUGAUGAGGCCGUUAGGCCGAA IAGAUGCU | 16059 |
| 3013 | AUCUCAUC U GUUACAGC | 6720 | GCUGUAAC CUGAUGAGGCCGUUAGGCCGAA IAUGAGAU | 16060 |
| 3019 | UCUGUUAC A GCUUCCAA | 6721 | UUGGAAGC CUGAUGAGGCCGUUAGGCCGAA IUAACAGA | 16061 |
| 3022 | GUUACAGC U UCCAAGUG | 6722 | CACUUGGA CUGAUGAGGCCGUUAGGCCGAA ICUGUAAC | 16062 |
| 3025 | ACAGCUUC C AAGUGGCU | 6723 | AGCCACUU CUGAUGAGGCCGUUAGGCCGAA IAAGCUGU | 16063 |
| 3026 | CAGCUUCC A AGUGGCUA | 6724 | UAGCCACU CUGAUGAGGCCGUUAGGCCGAA IGAAGCUG | 16064 |
| 3033 | CAAGUGGC U AAGGGCAU | 6725 | AUGCCCUU CUGAUGAGGCCGUUAGGCCGAA ICCACUUG | 16065 |
| 3040 | CUAAGGGC A UGGAGUUC | 6726 | GAACUCCA CUGAUGAGGCCGUUAGGCCGAA ICCCUUAG | 16066 |
| 3049 | UGGAGUUC U UGUCAUCG | 6727 | CGAUGCCA CUGAUGAGGCCGUUAGGCCGAA IAACUCCA | 16067 |
| 3054 | UUCUUGGC A UCGCGAAA | 6728 | UUUCGCGA CUGAUGAGGCCGUUAGGCCGAA ICCAAGAA | 16068 |
| 3070 | AGUGUAUC C ACAGGGAC | 6729 | GUCCCUGU CUGAUGAGGCCGUUAGGCCGAA IAUACACU | 16069 |
| 3071 | GUGUAUCC A CAGGGACC | 6730 | GGUCCCUG CUGAUGAGGCCGUUAGGCCGAA IGAUACAC | 16070 |
| 3073 | GUAUCCAC A GGGACCUG | 6731 | CAGGUCCC CUGAUGAGGCCGUUAGGCCGAA IUGGAUAC | 16071 |
| 3079 | ACAUGGAC C UGGCGGCA | 6732 | UGCCGCCA CUGAUGAGGCCGUUAGGCCGAA IUCCCUGU | 16072 |
| 3080 | CAGGGACC U GGCGGCAC | 6733 | GUGCCGCC CUGAUGAGGCCGUUAGGCCGAA IGUCCCUG | 16073 |
| 3087 | CUGGCGGC A CGAAAUAU | 6734 | AUAUUUCG CUGAUGAGGCCGUUAGGCCGAA ICCGCCAG | 16074 |
| 3097 | GAAAUAUC C UCUUAUCG | 6735 | CGAUAAGA CUGAUGAGGCCGUUAGGCCGAA IAUAUUUC | 16075 |
| 3098 | AAAUAUCC U CUUAUCGG | 6736 | CCGAUAAG CUGAUGAGGCCGUUAGGCCGAA IGAUAUUU | 16076 |
| 3100 | AUAUCCUC U UAUCGGAG | 6737 | CUCCGAUA CUGAUGAGGCCGUUAGGCCGAA IAGGAUAU | 16077 |
| 3127 | UUAAAAUC U GUGACUUU | 6738 | AAAGUCAC CUGAUGAGGCCGUUAGGCCGAA IAUUUUAA | 16078 |
| 3133 | UCUGUGAC U UUGGCUUG | 6739 | CAAGCCAA CUGAUGAGGCCGUUAGGCCGAA IUCACAGA | 16079 |
| 3139 | ACUUUGGC U UGGCCCGG | 6740 | CCGGGCCA CUGAUGAGGCCGUUAGGCCGAA ICCAAAGU | 16080 |
| 3144 | GGCUUGGC C CGGGAUAU | 6741 | AUAUCCCG CUGAUGAGGCCGUUAGGCCGAA ICCAAGCC | 16081 |
| 3145 | GCUUGGCC C GGGAUAUU | 6742 | AAUAUCCC CUGAUGAGGCCGUUAGGCCGAA IGCCAAGC | 16082 |
| 3164 | UAAAGAUC C AGAUUAUG | 6743 | CAUAAUCU CUGAUGAGGCCGUUAGGCCGAA IAUCUUUA | 16083 |
| 3165 | AAAGAUCC A GAUUAUGU | 6744 | ACAUAAUC CUGAUGAGGCCGUUAGGCCGAA IGAUCUUU | 16084 |
| 3175 | AUUAUGUC A GAAAAGGA | 6745 | UCCUUUUC CUGAUGAGGCCGUUAGGCCGAA IACAUAAU | 16085 |
| 3189 | GGAGAUGC U CGCCUCCC | 6746 | GGGAGGCG CUGAUGAGGCCGUUAGGCCGAA ICAUCUCC | 16086 |
| 3193 | AUCCUCGC C UCCCIUUG | 6747 | CAAAGGGA CUGAUGAGGCCGUUAGGCCGAA ICGAGCAU | 16087 |
| 3194 | UGCUCGCC U CCCUUUGA | 6748 | UCAAAGGG CUGAUGAGGCCGUUAGGCCGAA IGCGAGCA | 16088 |
| 3196 | CUCGCCUC C CUUUGAAA | 6749 | UUUCAAAG CUGAUGAGGCCGUUAGGCCGAA IAGCCGAC | 16089 |
| 3197 | UCGCCUCC C UUUGAAAU | 6750 | AUUUCAAA CUGAUGAGGCCGUUAGGCCGAA IGAGGCGA | 16090 |
| 3198 | CGCCUCCC U UUGAAAUG | 6751 | CAUUUCAA CUGAUGAGGCCGUUAGGCCGAA IGGAGGCG | 16091 |
| 3213 | UGGAUGGC C CCAGAAAC | 6752 | GUUUCUGG CUGAUGAGGCCGUUAGGCCGAA ICCAUCCA | 16092 |
| 3214 | GGAUGGCC C CACAAACA | 6753 | UGUUUCUG CUGAUGAGGCCGUUAGGCCGAA IGCCAUCC | 16093 |
| 3215 | GAUGGCCC C AGAAACAA | 6754 | UUGUUUCU CUGAUGAGGCCGUUAGGCCGAA IGGCCAUC | 16094 |
| 3216 | AUCGCCCC A GAAACAAU | 6755 | AUUGUUUC CUGAUGAGGCCGUUAGGCCGAA IGGGCCAU | 16095 |
| 3222 | CCAGAAAC A AUUUUUGA | 6756 | UCAAAAAU CUGAUGAGGCCGUUAGGCCGAA IUUUCUGG | 16096 |
| 3232 | UUUUUGAC A GAGUGUAC | 6757 | GUACACUC CUGAUGAGGCCGUUAGGCCGAA IUCAAAAA | 16097 |
| 3241 | GAGUGUAC A CAAUCCAG | 6758 | CUGGAUUG CUGAUGAGGCCGUUAGGCCGAA IUACACUC | 16098 |
| 3243 | GUGUACAC A AUCCAGAG | 6759 | CUCUGGAU CUGAUGAGGCCGUUAGGCCGAA IUGUACAC | 16099 |
| 3247 | ACACAAUC C AGAGUGAC | 6760 | GUCACUCU CUGAUGAGGCCGUUAGGCCGAA IAUUGUGU | 16100 |
| 3248 | CACAAUCC A GAGUGACG | 6761 | CGUCACUC CUGAUGAGGCCGUUAGGCCGAA IGAUUGUG | 16101 |
| 3259 | GUGACGUC U GGUCUUUU | 6762 | AAAAGACC CUGAUGAGGCCGUUAGGCCGAA IACGUCAC | 16102 |
| 3264 | GUCUGGUC U UUUGGUGU | 6763 | ACACCAAA CUGAUGAGGCCGUUAGGCCGAA IACCAGAC | 16103 |
| 3278 | UGUUUUGC U GUGGGAAA | 6764 | UUUCCCAC CUGAUGAGGCCGUUAGGCCGAA ICAAAACA | 16104 |
| 3294 | AUAUUUUC C UUAGGUGC | 6765 | GCACCUAA CUGAUGAGGCCGUUAGGCCGAA IAAAAUAU | 16105 |
| 3295 | UAUUUUCC U UAGGUGCU | 6766 | AGCACCUA CUGAUGAGGCCGUUAGGCCGAA IGAAAAUA | 16106 |
| 3303 | UUAGGUGC U UCUCCAUA | 6767 | UAUGGAGA CUGAUGAGGCCGUUAGGCCGAA ICACCUAA | 16107 |
| 3306 | GGUGCUUC U CCAUAUCC | 6768 | GGAUAUGG CUGAUGAGGCCGUUAGGCCGAA IAAGCACC | 16108 |
| 3308 | UGCUUCUC C AUAUCCUG | 6769 | CAGGAUAU CUGAUGAGGCCGUUAGGCCGAA IAGAAGCA | 16109 |
| 3309 | GCUUCUCC A UAUCCUGG | 6770 | CCAGGAUA CUGAUGAGGCCGUUAGGCCGAA IGAGAAGC | 16110 |
| 3314 | UCCAUAUC C UGGGGUAA | 6771 | UUACCCCA CUGAUGAGGCCGUUAGGCCGAA IAUAUGGA | 16111 |
| 3315 | CCAUAUCC U GGGGUAAA | 6772 | UUUACCCC CUGAUGAGGCCGUUAGGCCGAA IGAUAUGG | 16112 |
| 3363 | GAAGGAAC U AGAAUGAG | 6773 | CUCAUUCU CUGAUGAGGCCGUUAGGCCGAA IUUCCUUC | 16113 |
| 3375 | AUGAGGGC C CCUGAUUA | 6774 | UAAUCAGG CUGAUGAGGCCGUUAGGCCGAA ICCCUCAU | 16114 |
| 3376 | UGAGGGCC C CUGAUUAU | 6775 | AUAAUCAG CUGAUGAGGCCGUUAGGCCGAA IGCCCUCA | 16115 |
| 3377 | GAGGGCCC C UGAUUAUA | 6776 | UAUAAUCA CUGAUGAGGCCGUUAGGCCGAA IGGCCCUC | 16116 |
| 3378 | AGGGCCCC U GAUUAUAC | 6777 | GUAUAAUC CUGAUGAGGCCGUUAGGCCGAA IGGGCCCU | 16117 |
| 3387 | GAUUAUAC U ACACCAGA | 6778 | UCUGGUGU CUGAUGAGGCCGUUAGGCCGAA IUAUAAUC | 16118 |
| 3390 | UAUACUAC A CCAGAAAU | 6779 | AUUUCUGG CUGAUGAGGCCGUUAGGCCGAA IUAGUAUA | 16119 |
| 3392 | UACUACAC C AGAAAUGU | 6780 | ACAUUUCU CUGAUGAGGCCGUUAGGCCGAA IUGUAGUA | 16120 |
| 3393 | ACUACACC A GAAAUGUA | 6781 | UACAUUUC CUGAUGAGGCCGUUAGGCCGAA IGUGUAGU | 16121 |
| 3403 | AAAUGUAC C AGACCAUG | 6782 | CAUGGUCU CUGAUGAGGCCGUUAGGCCGAA IUACAUUU | 16122 |
| 3404 | AAUGUACC A GACCAUGC | 6783 | GCAUGGUC CUGAUGAGGCCGUUAGGCCGAA IGUACAUU | 16123 |
| 3408 | UACCAGAC C AUGCUGGA | 6784 | UCCAGCAU CUGAUGAGGCCGUUAGGCCGAA IUCUGGUA | 16124 |
| 3409 | ACCAGACC A UCCUGGAC | 6785 | GUCCAGCA CUGAUGAGGCCGUUAGGCCGAA IGUCUGGU | 16125 |
| 3413 | GACCAUGC U GGACUGCU | 6786 | AGCAGUCC CUGAUGAGGCCGUUAGGCCGAA ICAUGGUC | 16126 |
| 3418 | UGCUGGAC U GCUGGCAC | 4886 | GUGCCAGC CUGAUGAGGCCGUUAGGCCGAA IUCCACCA | 14226 |
| 3421 | UGGACUGC U GGCACGGG | 6787 | CCCGUGCC CUGAUGAGGCCGUUAGGCCGAA ICAGUCCA | 16127 |
| 3425 | CUGCUGGC A CGGGGAGC | 6788 | GCUCCCCG CUGAUGAGGCCGUUAGGCCGAA ICCAGCAG | 16128 |
| 3434 | CGGGGAGC C CAGUCAGA | 6789 | UCUGACUG CUGAUGAGGCCGUUAGGCCGAA ICUCCCCG | 16129 |
| 3435 | GGGGAGCC C AGUCAGAG | 6790 | CUCUGACU CUGAUGAGGCCGUUAGGCCGAA IGCUCCCC | 16130 |
| 3436 | GGGAGCCC A GUCAGAGA | 6791 | UCUCUGAC CUGAUGAGGCCGUUAGGCCGAA IGGCUCCC | 16131 |
| 3440 | GCCCAGUC A GAGACCCA | 6792 | UGGGUCUC CUGAUGAGGCCGUUAGGCCGAA IACUGGGC | 16132 |
| 3446 | UCAGAGAC C CACGUUUU | 6793 | AAAACGUG CUGAUGAGGCCGUUAGGCCGAA IUCUCUGA | 16133 |
| 3447 | CAGAGACC C ACGUUUUC | 6794 | GAAAACGU CUGAUGAGGCCGUUAGGCCGAA IGUCUCUG | 16134 |
| 3448 | AGAGACCC A CGUUUUCA | 6795 | UGAAAACG CUGAUGAGGCCGUUAGGCCGAA IGGUCUCU | 16135 |
| 3456 | ACGUUUUC A GAGUUGGU | 6796 | ACCAACUC CUGAUGAGGCCGUUAGGCCGAA IAAAACGU | 16136 |
| 3470 | GGUGCAAC A UUUGGGAA | 6797 | UUCCCAAA CUGAUGAGGCCGUUAGGCCGAA IUUCCACC | 16137 |
| 3482 | GCGAAAUC U CUUGCAAG | 6798 | CUUGCAAG CUGAUGAGGCCGUUAGGCCGAA IAUUUCCC | 16138 |
| 3484 | GAAAUCUC U UGCAAGCU | 6799 | AGCUUGCA CUGAUGAGGCCGUUAGGCCGAA IAGAUUUC | 16139 |
| 3488 | UCUCUUGC A AGCUAAUG | 6800 | CAUUACCU CUGAUGAGGCCGUUAGGCCGAA ICAAGAGA | 16140 |
| 3492 | UUGCAAGC U AAUGCUCA | 6801 | UGAGCAUU CUGAUGAGGCCGUUAGGCCGAA ICUUGCAA | 16141 |
| 3498 | GCUAAUCC U CAGCAGGA | 6802 | UCCUGCUC CUGAUGAGGCCGUUAGGCCGAA ICAUUAGC | 16142 |
| 3500 | UAAUGCUC A GCAGGAUG | 6803 | CAUCCUGC CUGAUGAGGCCGUUAGGCCGAA IAGCAUUA | 16143 |
| 3503 | UGCUCAGC A GGAUGGCA | 6804 | UGCCAUCC CUGAUGAGGCCGUUAGGCCGAA ICUGAUCA | 16144 |
| 3511 | AGGAUGGC A AAGACUAC | 6805 | GUAGUCUU CUGAUGAGGCCGUUAGGCCGAA ICCAUCCU | 16145 |
| 3517 | GCAAAGAC U ACAUUGUU | 6806 | AACAAUGU CUGAUGAGGCCGUUAGGCCGAA IUCUUUGC | 16146 |
| 3520 | AAGACUAC A UUGUUCUU | 6807 | AAGAACAA CUGAUGAGGCCGUUAGGCCGAA IUAGUCUU | 16147 |
| 3527 | CAUUGUUC U UCCGAUAU | 6808 | AUAUCGGA CUGAUGAGGCCGUUAGGCCGAA IAACAAUG | 16148 |
| 3530 | UGUUCUUC C GAUAUCAG | 6809 | CUGAUAUC CUGAUGAGGCCGUUAGGCCGAA IAAGAACA | 16149 |
| 3537 | CCCAUAUC A GAGACUUU | 6810 | AAAGUCUC CUGAUGAGGCCGUUAGGCCGAA IAUAUCGG | 16150 |
| 3543 | UCAGAGAC U UUGAGCAU | 6811 | AUGCUCAA CUGAUGAGGCCGUUAGGCCGAA IUCUCUGA | 16151 |
| 3550 | CUUUGAGC A UGGAAGAG | 6812 | CUCUUCCA CUGAUGAGGCCGUUAGGCCGAA ICUCAAAG | 16152 |
| 3564 | GAGGAGUC U UGACUCUC | 6813 | GAGAGUCC CUGAUGAGGCCGUUAGGCCGAA IAAUCCUC | 16153 |
| 3569 | UUCUGGAC U CUCUCUGC | 6814 | GCAGAGAG CUGAUGAGGCCGUUAGGCCGAA IUCCAGAA | 16154 |
| 3571 | CUGGACUC U CUCUGCCU | 6815 | AGGCAGAG CUGAUGAGGCCGUUAGGCCGAA IAGUCCAG | 16155 |
| 3573 | GGACUCUC U CUGCCUAC | 6816 | GUAGGCAG CUGAUGAGGCCGUUAGGCCGAA IAGAGUCC | 16156 |
| 3575 | ACUCUCUC U GCCUACCU | 6817 | AGGUAGGC CUGAUGAGGCCGUUAGGCCGAA IAGAGAGU | 16157 |
| 3578 | CUCUCUGC C UACCUCAC | 6818 | GUGAGGUA CUGAUGAGGCCGUUAGGCCGAA ICAGAGAG | 16158 |
| 3579 | UCUCUGCC U ACCUCACC | 6819 | GGUGAGGU CUGAUGAGGCCGUUAGGCCGAA IGCAGAGA | 16159 |
| 3582 | CUGCCUAC C UCACCUGU | 6820 | ACAGGUGA CUGAUGAGGCCGUUAGGCCGAA IUAGGCAG | 16160 |
| 3583 | UGCCUACC U CACCUGUU | 6821 | AACAGGUG CUGAUGAGGCCGUUAGGCCGAA IGUAGGCA | 16161 |
| 3585 | CCUACCUC A CCUGUUUC | 6822 | GAAACAGG CUGAUGAGGCCGUUAGGCCGAA IAGGUAGG | 16162 |
| 3587 | UACCUCAC C UGUUUCCU | 6823 | AGGAAACA CUGAUGAGGCCGUUAGGCCGAA IUGAGGUA | 16163 |
| 3588 | ACCUCACC U GUUUCCUG | 6824 | CAGGAAAC CUGAUGAGGCCGUUAGGCCGAA IGUGAGGU | 16164 |
| 3594 | CCUGUUUC C UGUAUGGA | 6825 | UCCAUACA CUGAUGAGGCCGUUAGGCCGAA IAAACAGG | 16165 |
| 3595 | CUGUUUCC U GUAUGGAG | 6826 | CUCCAUAC CUGAUGAGGCCGUUAGGCCGAA IGAAACAG | 16166 |
| 3622 | UAUGUGAC C CCAAAUUC | 6827 | GAAUUUGG CUGAUGAGGCCGUUAGGCCGAA IUCACAUA | 16167 |
| 3623 | AUGUGACC C CAAAUUCC | 6828 | GGAAUUUG CUGAUGAGGCCGUUAGGCCGAA IGUCACAU | 16168 |
| 3624 | UGUGACCC C AAAUUCCA | 6829 | UGGAAUUU CUGAUGAGGCCGUUAGGCCGAA IGGUCACA | 16169 |
| 3625 | GUGACCCC A AAUUCCAU | 6830 | AUGGAAUU CUGAUGAGGCCGUUAGGCCGAA IGGGUCAC | 16170 |
| 3631 | CCAAAUUC C AUUAUGAC | 6831 | GUCAUAAU CUGAUGAGGCCGUUAGGCCGAA IAAUUUGG | 16171 |
| 3632 | CAAAUUCC A UUAUGACA | 6832 | UGUCAUAA CUGAUGAGGCCGUUAGGCCGAA IGAAUUUG | 16172 |
| 3640 | AUUAUGAC A ACACAGCA | 6833 | UGCUGUGU CUGAUGAGGCCGUUAGGCCGAA IUCAUAAU | 16173 |
| 3643 | AUGACAAC A CAGCAGGA | 6834 | UCCUGCUG CUGAUGAGGCCGUUAGGCCGAA IUUGUCAU | 16174 |
| 3645 | GACAACAC A GCAGGAAU | 6835 | AUUCCUGC CUGAUGAGGCCGUUAGGCCGAA IUGUUGUC | 16175 |
| 3648 | AACACAGC A GGAAUCAG | 6836 | CUGAUUCC CUGAUGAGGCCGUUAGGCCGAA ICUGUGUU | 16176 |
| 3655 | CAGGAAUC A GUCAGUAU | 6837 | AUACUGAC CUGAUGAGGCCGUUAGGCCGAA IAUUCCUG | 16177 |
| 3659 | AAUCAGUC A GUAUCUGC | 6838 | GCAGAUAC CUGAUGAGGCCGUUAGGCCGAA IACUGAUU | 16178 |
| 3665 | UCAGUAUC U GCAGAACA | 6839 | UGUUCUGC CUGAUGAGGCCGUUAGGCCGAA IAUACUGA | 16179 |
| 3668 | GUAUCUGC A GAACAGUA | 6840 | UACUGUUC CUGAUGAGGCCGUUAGGCCGAA ICAGAUAC | 16180 |
| 3673 | UGCAGAAC A GUAAGCGA | 6841 | UCGCUUAC CUGAUGAGGCCGUUAGGCCGAA IUUCUGCA | 16181 |
| 3688 | GAAAGAGC C GGCCUGUG | 6842 | CACAGGCC CUGAUGAGGCCGUUAGGCCGAA ICUCUUUC | 16182 |
| 3692 | GAGCCGGC C UGUGAGUG | 6843 | CACUCACA CUGAUGAGGCCGUUAGGCCGAA ICCGGCUC | 16183 |
| 3693 | AGCCGGCC U GUGAGUGU | 6844 | ACACUCAC CUGAUGAGGCCGUUAGGCCGAA IGCCGGCU | 16184 |
| 3708 | GUAAAAAC A UUUGAAGA | 6845 | UCUUCAAA CUGAUGAGGCCGUUAGGCCGAA IUUUUUAC | 16185 |
| 3721 | AAGAUAUC C CGUUAGAA | 6846 | UUCUAACG CUGAUGAGGCCGUUAGGCCGAA IAUAUCUU | 16186 |
| 3722 | AGAUAUCC C GUUAGAAG | 6847 | CUUCUAAC CUGAUGAGGCCGUUAGGCCGAA IGAUAUCU | 16187 |
| 3734 | AGAAGAAC C AGAAGUAA | 6848 | UUACUUCU CUGAUGAGGCCGUUAGGCCGAA IUUCUUCU | 16188 |
| 3735 | GAAGAACC A GAAGUAAA | 6849 | UUUACUUC CUGAUGAGGCCGUUAGGCCGAA IGUUCUUC | 16189 |
| 3751 | AACUAAUC C CAGAUGAC | 6850 | GUCAUCUG CUGAUGAGGCCGUUAGGCCGAA IAUUACUU | 16190 |
| 3752 | AGUAAUCC C AGAUGACA | 6851 | UGUCAUCU CUGAUGAGGCCGUUAGGCCGAA IGAUUACU | 16191 |
| 3753 | GUAAUCCC A GAUGACAA | 6852 | UUGUCAUC CUGAUGAGGCCGUUAGGCCGAA IGGAUUAC | 16192 |
| 3760 | CAGAUGAC A ACCAGACG | 6853 | CGUCUGGU CUGAUGAGGCCGUUAGGCCGAA IUCAUCUG | 16193 |
| 3763 | AUGACAAC C AGACGGAC | 6854 | GUCCGUCU CUGAUGAGGCCGUUAGGCCGAA IUUGUCAU | 16194 |
| 3764 | UGACAACC A GACGGACA | 6855 | UGUCGGUC CUGAUGAGGCCGUUAGGCCGAA IGUUGUCA | 16195 |
| 3772 | AGACGGAC A GUGGUAUG | 6856 | CAUACCAC CUGAUGAGGCCGUUAGGCCGAA IUCCGUCU | 16196 |
| 3785 | UAUGGUUC U UGCCUCAG | 6857 | CUGAGGCA CUGAUGAGGCCGUUAGGCCGAA IAACCAUA | 16197 |
| 3789 | GUUCUUGC C UCAGAAGA | 6858 | UCUUCUGA CUGAUGAGGCCGUUAGGCCGAA ICAAGAAC | 16198 |
| 3790 | UUCUUGCC U CAGAAGAG | 6859 | CUCUUCUG CUGAUGAGGCCGUUAGGCCGAA IGCAAGAA | 16199 |
| 3792 | CUUGCCUC A GAAGAGCU | 6860 | AGCUCUUC CUGAUGAGGCCGUUAGGCCGAA IAGGCAAG | 16200 |
| 3800 | AGAAGAGC U GAAAACUU | 6861 | AAGUUUUC CUGAUGAGGCCGUUAGGCCGAA ICUCUUCU | 16201 |
| 3807 | CUGAAAAC U UUGGAAGA | 6862 | UCUUCCAA CUGAUGAGGCCGUUAGGCCGAA IUUUUCAG | 16202 |
| 3817 | UGGAAGAC A GAACCAAA | 6863 | UUUGGUUC CUGAUGAGGCCGUUAGGCCGAA IUCUUCCA | 16203 |
| 3822 | GACAGAAC C AAAUUAUC | 6864 | GAUAAUUU CUGAUGAGGCCGUUAGGCCGAA IUUCUGUC | 16204 |
| 3823 | ACAGAACC A AAUUAUCU | 6865 | AGAUAAUU CUGAUGAGGCCGUUAGGCCGAA IGUUCUGU | 16205 |
| 3831 | AAAUUAUC U CCAUCUUU | 6866 | AAAGAUGG CUGAUGAGGCCGUUAGGCCGAA IAUAAUUU | 16206 |
| 3833 | AUUAUCUC C AUCUUUUG | 6867 | CAAAAGAU CUGAUGAGGCCGUUAGGCCGAA IAGAUAAU | 16207 |
| 3834 | UUAUCUCC A UCUUUUGG | 6868 | CCAAAAGA CUGAUGAGGCCGUUAGGCCGAA IGAGAUAA | 16208 |
| 3837 | UCUCCAUC U UUUGGUGG | 6869 | CCACCAAA CUGAUGAGGCCGUUAGGCCGAA IAUGGAGA | 16209 |
| 3854 | AAUGGUGC C CAGCAAAA | 6870 | UUUUGCUG CUGAUGAGGCCGUUAGGCCGAA ICACCAUU | 16210 |
| 3855 | AUGGUGCC C AGCAAAAG | 6871 | CUUUUGCU CUGAUGAGGCCGUUAGGCCGAA IGCACCAU | 16211 |
| 3856 | UGGUGCCC A GCAAAAGC | 6872 | GCUUUUGC CUGAUGAGGCCGUUAGGCCGAA IGGCACCA | 16212 |
| 3859 | UGCCCAGC A AAAGCAGG | 6873 | CCUGCUUU CUGAUGAGGCCGUUAGGCCGAA ICUGGGCA | 16213 |
| 3865 | GCAAAAGC A GGGAGUCU | 6874 | AGACUCCC CUGAUGAGGCCGUUAGGCCGAA ICUUUUGC | 16214 |
| 3873 | AGGGAGUC U GUGGCAUC | 6875 | GAUGCCAC CUGAUGAGGCCGUUAGGCCGAA IACUCCCU | 16215 |
| 3879 | UCUGUGGC A UCUGAAGG | 6876 | CCUUCAGA CUGAUGAGGCCGUUAGGCCGAA ICCACAGA | 16216 |
| 3882 | GUGGCAUC U GAAGGCUC | 6877 | GAGCCUUC CUGAUGAGGCCGUUAGGCCGAA IAUGCCAC | 16217 |
| 3889 | CUGAAGGC U CAAACCAG | 6878 | CUGGUUUG CUGAUGAGGCCGUUAGGCCGAA ICCUUCAG | 16218 |
| 3891 | GAAGGCUC A AACCAGAC | 6879 | GUCUGGUU CUGAUGAGGCCGUUAGGCCGAA IAGCCUUC | 16219 |
| 3895 | GCUCAAAC C AGACAAGC | 6880 | GCUUGUCU CUGAUGAGGCCGUUAGGCCGAA IUUUGAGC | 16220 |
| 3896 | CUCAAACC A GACAAGCG | 6881 | CGCUUGUC CUGAUGAGGCCGUUAGGCCGAA IGUUUGAG | 16221 |
| 3900 | AACCAGAC A AGCGGCUA | 6882 | UAGCCGCU CUGAUGAGGCCGUUAGGCCGAA IUCUGGUG | 16222 |
| 3907 | CAAGCGGC U ACCAGUCC | 6883 | GGACUGGU CUGAUGAGGCCGUUAGGCCGAA ICCGCUUG | 16223 |
| 3910 | GCGGCUAC C AGUCCGGA | 6884 | UCCGGACU CUGAUGAGGCCGUUAGGCCGAA IUAGCCGC | 16224 |
| 3911 | CGGCUACC A GUCCGGAU | 6885 | AUCCGGAC CUGAUGAGGCCGUUAGGCCGAA IGUAGCCG | 16225 |
| 3915 | UACCAGUC C GGAUAUCA | 6886 | UGAUAUCC CUGAUGAGGCCGUUAGGCCGAA IACUGGUA | 16226 |
| 3923 | CGGAUAUC A CUCCGAUG | 6887 | CAUCGGAG CUGAUGAGGCCGUUAGGCCGAA IAUAUCCG | 16227 |
| 3925 | GAHAUCAC U CCGAUGAC | 6888 | GUCAUCGG CUGAUGAGGCCGUUAGGCCGAA IUGAUAUC | 16228 |
| 3927 | UAUCACUC C GAUGACAC | 6889 | GUGUCAUC CUGAUGAGGCCGUUAGGCCGAA IAGUGAUA | 16229 |
| 3934 | CCGAUGAC A CAGACACC | 6890 | GGUGUCUG CUGAUGAGGCCGUUAGGCCGAA IUCAUCGG | 16230 |
| 3936 | GAUGACAC A GACACCAC | 6891 | GUGGUGUC CUGAUGAGGCCGUUAGGCCGAA IUGUCAUC | 16231 |
| 3940 | ACACAGAC A CCACCGUG | 6892 | CACGGUGG CUGAUGAGGCCGUUAGGCCGAA IUCUGUGU | 16232 |
| 3942 | ACAGACAC C ACCGUGUA | 6893 | UACACGGU CUGAUGAGGCCGUUAGGCCGAA IUGUCUCU | 16233 |
| 3943 | CACACACC A CCGUGUAC | 6894 | GUACACGG CUGAUGAGGCCGUUAGGCCGAA IGUGUCUG | 16234 |
| 3945 | GACACCAC C GUGUACUC | 6895 | GAGUACAC CUGAUGAGGCCGUUAGGCCGAA IUGGUGUC | 16235 |
| 3952 | CCGUGUAC U CCAGUGAG | 6896 | CUCACUGG CUGAUGAGGCCGUUAGGCCGAA IUACACUG | 16236 |
| 3954 | GUGUACUC C AGUGAGGA | 6897 | UCCUCACU CUGAUGAGGCCGUUAGGCCGAA IAGUACAC | 16237 |
| 3955 | UGUACUCC A GUGAGGAA | 6898 | UUCCUCAC CUGAUGAGGCCGUUAGGCCGAA IGAGUACA | 16238 |
| 3966 | GAGGAAGC A GAACUUUU | 6899 | AAAAGUUC CUGAUGAGGCCGUUAGGCCGAA ICUUCCUC | 16239 |
| 3971 | AGCAGAAC U UUUAAAGC | 6900 | GCUUUAAA CUGAUGAGGCCGUUAGGCCGAA IUUCUGCU | 16240 |
| 3980 | UUUAAAGC U GAGAGAGA | 6901 | UCUCUAUC CUGAUGAGGCCGUUAGGCCGAA ICUUUAAA | 16241 |
| 3998 | UGGAGUGC A AACCGGUA | 6902 | UACCGGUU CUGAUGAGGCCGUUAGGCCGAA ICACUCCA | 16242 |
| 4002 | GUGCAAAC C GGUAGCAC | 6903 | GUGCUACC CUGAUGAGGCCGUUAGGCCGAA IUUUGCAC | 16243 |
| 4009 | CCGGUAGC A CAGCCCAG | 6904 | CUGGGCUG CUGAUGAGGCCGUUAGGCCGAA ICUACCGG | 16244 |
| 4011 | GGUAGCAC A GCCCAGAU | 6905 | AUCUGGGC CUGAUGAGGCCGUUAGGCCGAA IUGCUACC | 16245 |
| 4014 | AGCACAGC C CAGAUUCU | 6906 | AGAAUCUG CUGAUGAGGCCGUUAGGCCGAA ICUGUGCU | 16246 |
| 4015 | GCACAGCC C AGAUUCUC | 6907 | GAGAAUCU CUGAUGAGGCCGUUAGGCCGAA IGCUGUGC | 16247 |
| 4016 | CACAGCCC A GAUUCUCC | 6908 | GGAGAAUC CUGAUGAGGCCGUUAGGCCGAA IGGCUGUG | 16248 |
| 4022 | CCAGAUUC U CCAGCCUG | 6909 | CAGGCUGG CUGAUGAGGCCGUUAGGCCGAA IAAUCUGG | 16249 |
| 4024 | AGAUUCUC C AGCCUGAC | 6910 | GUCAGGCU CUGAUGAGGCCGUUAGGCCGAA IAGAAUCU | 16250 |
| 4025 | GAUUCUCC A GCCUGACA | 6911 | UGUCAGGC CUGAUGAGGCCGUUAGGCCGAA IGAGAAUC | 16251 |
| 4028 | UCUCCAGC C UGACAGGG | 6912 | CCGUGUCA CUGAUGAGGCCGUUAGGCCGAA ICUGGAGA | 16252 |
| 4029 | CUCCAGCC U GACACGGG | 6913 | CCCGUGUC CUGAUGAGGCCGUUAGGCCGAA ICCUGGAG | 16253 |
| 4033 | AGCCUGAC A CGGGGACC | 6914 | GGUCCCCG CUGAUGAGGCCGUUAGGCCGAA IUCAGGCU | 16254 |
| 4041 | ACGGGGAC C ACACUGAG | 6915 | CUCAGUGU CUGAUGAGGCCGUUAGGCCGAA IUCCCCGU | 16255 |
| 4042 | CGGGGACC A CACUGAGC | 6916 | GCUCAGUG CUGAUGAGGCCGUUAGGCCGAA IGUCCCCG | 16256 |
| 4044 | GGGACCAC A CUGACCUC | 6917 | GAGCUCAG CUGAUGAGGCCGUUAGGCCGAA IUGGUCCC | 16257 |
| 4046 | GACCACAC U GACCUCUC | 6918 | GAGACCUC CUGAUGAGGCCGUUAGGCCGAA IUGUGGUC | 16258 |
| 4051 | CACUGAGC U CUCCUCCU | 6919 | AGGAGGAG CUGAUGAGGCCGUUAGGCCGAA ICUCAGUG | 16259 |
| 4053 | CUGAGCUC U CCUCCUGU | 6920 | ACAGGAGG CUGAUGAGGCCGUUAGGCCGAA IAGCUCAG | 16260 |
| 4055 | GAGCUCUC C UCCUGUUU | 6921 | AAACAGGA CUGAUGAGGCCGUUAGGCCGAA IAGAGCUC | 16261 |
| 4056 | AGCUCUCC U CCUGUUUA | 6922 | UAAACAGG CUGAUGAGGCCGUUAGGCCGAA IGAGAGCU | 16262 |
| 4058 | CUCUCCUC C UGUUUAAA | 6923 | UUUAAACA CUGAUGAGGCCGUUAGGCCGAA IAGGAGAG | 16263 |
| 4059 | UCUCCUCC U GUUUAAAA | 6924 | UUUUAAAC CUGAUGAGGCCGUUAGGCCGAA IGAGGAGA | 16264 |
| 4074 | AAGGAAGC A UCCACACC | 6925 | GGUGUGGA CUGAUGAGGCCGUUAGGCCGAA ICUUCCUU | 16265 |
| 4077 | GAAGCAUC C ACACCCCA | 6926 | UGGGGUGU CUGAUGAGGCCGUUAGGCCGAA IAUCCUUC | 16266 |
| 4078 | AAGCAUCC A CACCCCAA | 6927 | UUGGGGUG CUGAUGAGGCCGUUAGGCCGAA IGAUGCUU | 16267 |
| 4080 | GCAUCCAC A CCCCAACU | 6928 | AGUUGGGG CUGAUGAGGCCGUUAGGCCGAA IUGGAUGC | 16268 |
| 4082 | AUCCACAC C CCAACUCC | 6929 | GGAGUUGG CUGAUGAGGCCGUUAGGCCGAA IUGUGGAU | 16269 |
| 4083 | UCCACACC C CAACUCCC | 6930 | GGGAGUUG CUGAUGAGGCCGUUAGGCCGAA IGUGUGGA | 16270 |
| 4084 | CCACACCC C AACUCCCG | 6931 | CGGGAGUU CUGAUGAGGCCGUUAGGCCGAA IGGUGUGG | 16271 |
| 4085 | CACACCCC A ACUCCCGG | 6932 | CCGGGAGU CUGAUGAGGCCGUUAGGCCGAA IGGGUGUG | 16272 |
| 4088 | ACCCCAAC U CCCGGACA | 6933 | UGUCCGGG CUGAUGAGGCCGUUAGGCCGAA IUUGGGGU | 16273 |
| 4090 | CCCAACUC C CGGACAUC | 6934 | GAUGUCCG CUGAUGAGGCCGUUAGGCCGAA IAGUUGGG | 16274 |
| 4091 | CCAACUCC C GGACAUCA | 6935 | UGAUGUCC CUGAUGAGGCCGUUAGGCCGAA IGAGUUGG | 16275 |
| 4096 | UCCCGGAC A UCACAUGA | 6936 | UCAUGUGA CUGAUGAGGCCGUUAGGCCGAA IUCCGGGA | 16276 |
| 4099 | CGGACAUC A CAUGAGAG | 6937 | CUCUCAUG CUGAUGAGGCCGUUAGGCCGAA IAUGUCCG | 16277 |
| 4101 | GACAUCAC A UGAGAGGU | 6938 | ACCUCUCA CUGAUGAGGCCGUUAGGCCGAA IUGAUGUC | 16278 |
| 4111 | GAGAGGUC U GCUCAGAU | 6939 | AUCUGAGC CUGAUGAGGCCGUUAGGCCGAA IACCUCUC | 16279 |
| 4114 | AGGUCUGC U CAGAUUUU | 6940 | AAAAUCUG CUGAUGAGGCCGUUAGGCCGAA ICAGACCU | 16280 |
| 4116 | GUCUCCUC A GAUUUUGA | 6941 | UCAAAAUC CUGAUGAGGCCGUUAGGCCGAA IAGCAGAC | 16281 |
| 4135 | UCUUGUUC U UUCCACCA | 6942 | UGGUGGAA CUGAUGAGGCCGUUAGGCCGAA IAACAACA | 16282 |
| 4139 | GUUCUUUC C ACCAGCAG | 6943 | CUGCUGGU CUGAUGAGGCCGUUAGGCCGAA IAAAGAAC | 16283 |
| 4140 | UUCUUUCC A CCAGCAGG | 6944 | CCUGCUGG CUGAUGAGGCCGUUAGGCCGAA ICAAAGAA | 16284 |
| 4142 | CUUUCCAC C AGCAGGAA | 6945 | UUCCUGCU CUGAUGAGGCCGUUAGGCCGAA IUGGAAAG | 16285 |
| 4143 | UUUCCACC A GCAGGAAG | 6946 | CUUCCUCC CUGAUGAGGCCGUUAGGCCGAA IGUGGAAA | 16286 |
| 4146 | CCACCAGC A GGAAGUAG | 6947 | CUACUUCC CUGAUGAGGCCGUUAGGCCGAA ICUGGUGG | 16287 |
| 4156 | GAAGUAGC C GCAUUUGA | 6948 | UCAAAUGC CUGAUGAGGCCGUUAGGCCGAA ICUACUUC | 16288 |
| 4159 | GUAGCCGC A UUUGAUUU | 6949 | AAAUCAAA CUGAUGAGGCCGUUAGGCCGAA ICGGCUAC | 16289 |
| 4170 | UGAUUUUC A UUUCGACA | 6950 | UGUCGAAA CUGAUGAGGCCGUUAGGCCGAA IAAAAUCA | 16290 |
| 4178 | AUUUCGAC A ACACAAAA | 6951 | UUUUCUGU CUGAUGAGGCCGUUAGGCCGAA IUCGAAAU | 16291 |
| 4181 | UCGACAAC A GAAAAAGC | 6952 | CCUUUUUC CUGAUGAGGCCGUUAGGCCGAA IUUCUCGA | 16292 |
| 4192 | AAAAGGAC C UCGGACUG | 6953 | CAGUCCGA CUGAUGAGGCCGUUAGGCCGAA IUCCUUUU | 16293 |
| 4193 | AAAGGACC U CGGACUGC | 6954 | GCAGUCCG CUGAUGAGGCCGUUAGGCCGAA IGUCCUUU | 16294 |
| 4199 | CCUCGGAC U GCAGGGAG | 6955 | CUCCCUGC CUGAUGAGGCCGUUAGGCCGAA IUCCGAGG | 16295 |
| 4202 | CGGACUGC A UGGAUCCA | 6956 | UGGCUCCC CUGAUGAGGCCGUUAGGCCGAA ICAGUCCG | 16296 |
| 4209 | CAGGGAGC C AGCUCUUC | 6957 | GAAGAGCU CUGAUGAGGCCGUUAGGCCGAA ICUCCCUG | 16297 |
| 4210 | AGGGAGCC A GCUCUUCU | 6958 | AGAAGAGC CUGAUGAGGCCGUUAGGCCGAA IGCUCCCU | 16298 |
| 4213 | GAGCCAGC U CUUCUAGG | 6959 | CCUAGAAG CUGAUGAGGCCGUUAGGCCGAA ICUGGCUC | 16299 |
| 4215 | GCCAGCUC U UCUAGGCU | 6960 | AGCCUAGA CUGAUGAGGCCGUUAGGCCGAA IAGCUGGC | 16300 |
| 4218 | AGCUCUUC U AGGCUUGU | 6961 | ACAAGCCU CUGAUGAGGCCGUUAGGCCGAA IAAGAGCU | 16301 |

Core Sequence = CUGAUGAG GCCGUUAGGC CGAA.
Underlined region can be any X sequence or linker, as described herein.
“I” represents Inosine

TABLE XVII
KDR G-Cleaver Ribozyme and Target Sequences

| | | Seq ID | | Seq ID |
|---|---|---|---|---|
| Pos | Target | No | Ribozyme Sequence | No |
| 12 | GCAAGGUGCU G CUGGC | 6962 | GCCAG UGAUG GCAUGCACUAUGC GCG AGCACCUUGC | 16302 |
| 22 | GCUGGCCGUC G CCCUG | 6963 | CAGGG UGAUG GCAUGCACUAUGC GCG GACGGCCAGC | 16303 |
| 35 | CUGUGGCUCU G CGUGG | 6964 | CCACG UGAUG GCAUGCACUAUGC GCG AGAGCCACAG | 16304 |
| 52 | GACCCGGGCC G CCUCU | 6965 | AGAGG UGAUG GCAUGCACUAUGC GCG GGCCCGGGUC | 16305 |
| 66 | CUGUGGGUUU G CCUAG | 6966 | CUAGG UGAUG GCAUGCACUAUGC GCG AAACCCACAG | 16306 |
| 82 | UGUUUCUCUU G AUCUG | 6967 | CAGAU UGAUG GCAUGCACUAUGC GCG AAGAGAAACA | 16307 |
| 87 | CUCUUGAUGU G CCCAG | 6968 | CUGCC UGAUG GCAUGCACUAUGC GCG ACAUCAACAC | 16308 |
| 152 | CAAAUUACUU G CAGCG | 6969 | CCCUG UGAUG GCAUGCACUAUGC GCG AACUAAUUUG | 16309 |
| 205 | CAGUCCCAGU G ACCAA | 6970 | UUGCU UGAUG GCAUGCACUAUGC GCG ACUCCCACUC | 16310 |
| 222 | CCCUGGAGGU G ACUGA | 6971 | UCACU UGAUG GCAUGCACUAUGC CGG ACCUCCACCC | 16311 |
| 226 | CGAGGUGACU G AGUGC | 6972 | GCACU UGAUG GCAUGCACUAUGC GCG AGUCACCUCC | 16312 |
| 230 | GUCACUCAGU G CACCC | 6973 | CGCUG UGAUG GCAUGCACUAUGC GCG ACUCACUCAC | 16313 |
| 235 | UGACUCCAGC G AUCGC | 6974 | CCCAU UGAUG GCAUGCACUAUGC GCG CCUGCACUCA | 16314 |
| 273 | UUCCAAAAGU G AUCGG | 6975 | CCCAU UGAUG GCAUGCACUAUGC GCG ACUUUUGGAA | 16315 |
| 283 | GAUCGCAAAU G ACACU | 6976 | AGUCU UGAUG GCAUGCACUAUGC GCG AUUUCCCAUC | 16316 |
| 302 | CCCUACAAGU G CUUCU | 6977 | AGAAG UGAUG GCAUGCACUAUGC GCG ACUUCUACCC | 16317 |
| 319 | CCGCCAAACU G ACUUC | 6978 | CAACU UGAUG GCAUGCACUAUGC GCG ACUUUCCCCG | 16318 |
| 373 | UCCAUUUAUU G CUUCU | 6979 | ACAAC UGAUG GCAUGCACUAUGC GCG AAUAAAUGGA | 16319 |
| 385 | UUCUGUUAGU G ACCAA | 6980 | UUCGU UGAUG GCAUGCACUAUGC GCG ACUAACAGAA | 16320 |
| 412 | GUACAUUACU G ACAAC | 6981 | CUUCU UGAUG GCAUGCACUAUGC GCG AGUAAUCUAC | 16321 |
| 435 | AAACUCUCCU G AUUCC | 6982 | GCAAU UGAUG GCAUGCACUAUGC GCG ACCACAGUUU | 16322 |
| 481 | CUCACUUUCU G CAAGA | 6983 | UCUUC UGAUG GCAUGCACUAUGC GCG ACAAAGUGAC | 16323 |
| 511 | AUUUCUUCCU G AUCCU | 6984 | ACCAU UGAUG GCAUGCACUAUGC GCG ACCAACAAAU | 16324 |
| 567 | CCACCUACAU G AUCAC | 6985 | CUCAU UGAUG GCAUGCACUAUGC GCG AUCUACCUCC | 16325 |
| 577 | CAUCACCUAU G CUCCC | 6986 | CCCAC UGAUG GCAUGCACUAUGC GCG AUACCUGAUG | 16326 |
| 595 | CCUCUUCUCU G AACCA | 6987 | UCCUU UGAUG GCAUGCACUAUGC GCG ACACAACACC | 16327 |
| 610 | AAAAAUUAAU G AUCAA | 6988 | UUCAU UGAUG GCAUGCACUAUGC GCG AUUAAUUUUU | 16328 |
| 613 | AAUUAAUCAU G AAACU | 6989 | ACUUU UGAUG GCAUGCACUAUGC GCG AUCAUUAAUU | 16329 |
| 667 | UACCAUUUAU G AUCUC | 6990 | CACAC UGAUG GCAUGCACUAUGC GCG AUAAAUCCUA | 16330 |
| 678 | AUCUCCUUCU G ACUCC | 6991 | CCACU UGAUG GCAUGCACUAUGC GCG ACAACCACAU | 16331 |
| 697 | UGAUGCAAUU G AACUA | 6992 | UACUU UGAUG GCAUGCACUAUGC GCG AAUUCCAUCA | 16332 |
| 745 | ACCAACAACU G AACUA | 6993 | UACUU UGAUG GCAUGCACUAUGC GCG ACUUCUUCCU | 16333 |
| 763 | UCUCCCCAUU G ACUUC | 6994 | CAACU UGAUG GCAUGCACUAUGC GCG AAUCCCCACA | 16334 |
| 789 | ACCCUUCUUC G AACCA | 6995 | UCCUU UGAUG GCAUGCACUAUGC GCG CAACAACCCU | 16335 |
| 818 | CUUCUAAACC G ACACC | 6996 | CCUCU UGAUG GCAUGCACUAUGC GCG CCUUUACAAC | 16336 |
| 844 | CUCUCCCACU G ACAUC | 6997 | CAUCU UGAUG GCAUGCACUAUGC GCG ACUCCCACAC | 16337 |
| 849 | CCACUCACAU G AACAA | 6998 | UUCUU UGAUG GCAUGCACUAUGC GCG AUCUCACUCC | 16338 |
| 861 | ACAAAUUUUU G ACCAC | 6999 | CUCCU UGAUG GCAUGCACUAUGC GCG AAAAAUUUCU | 16339 |
| 895 | AACCCCCACU G ACCAA | 7000 | UUCCU UGAUG GCAUGCACUAUGC GCG ACUCCCCCUU | 16340 |
| 916 | CUACACCUCU G CACCA | 7001 | UCCUC UGAUG GCAUGCACUAUGC GCG ACACCUCUAC | 16341 |
| 933 | CCACUCCCCU G AUCAC | 7002 | CUCAC UGAUG GCAUGCACUAUGC GCG ACCCCACUCC | 16342 |
| 936 | CUCCCCUCAU G ACCAA | 7003 | UUCCU UGAUG GCAUGCACUAUGC GCG AUCACCCCAC | 16343 |
| 970 | CACCCUCCAU G AAAAA | 7004 | UUUUU UGAUG GCAUGCACUAUGC GCG AUCCACCCUC | 16344 |
| 985 | ACCUUUUCUU G CUUUU | 7005 | AAAAC UGAUG GCAUGCACUAUGC GCG AACAAAACCU | 16345 |
| 1048 | CACAAUCCCU G CCAAC | 7006 | CUUCC UGAUG GCAUGCACUAUGC GCG ACCCAUUCUC | 16346 |
| 1050 | CAAUCCCUCC G AAGUA | 7007 | UACUU UGAUG GCAUGCACUAUGC GCG CCACCCAUUC | 16347 |
| 1108 | AAUACCCCUU G AGUCC | 7008 | GGACU UGAUC GCAUGCACUAUGC GCG AAGGGCUAUU | 16348 |
| 1143 | GGCAUGUACU G ACGAU | 7009 | AUCGU UGAUG GCAUCCACUAUGC GCG AGUACAUGCC | 16349 |
| 1146 | AUGUACUGAC G AUUAU | 7010 | AUAAU UGAUG GCAUGCACUAUGC GCG GUCAGUACAU | 16350 |
| 1158 | UUAUCCAAGU G AGUGA | 7011 | UCACU UGAUG GCAUGCACUAUCC GCC ACUUCCAUAA | 16351 |
| 1162 | GCAAGUGAGU G AAAGA | 7012 | UCUUU UGAUG GCAUCCACUAUCC GCG ACUCACUUCC | 16352 |
| 1267 | CCACAUUGCU G AGAAA | 7013 | UUUCU UGAUG GCAUGCACUAUGC GCC ACCAAUCUGG | 16353 |
| 1320 | CCACUCAAAC G CUGAC | 7014 | GUCAC UGAUG GCAUGCACUAUGC CCG GUUUCAGUGC | 16354 |
| 1323 | CUCAAACCCU G ACAUC | 7015 | CAUGU UGAUG GCAUGCACUAUGC GCG AGCCUUUCAC | 16355 |
| 1339 | UACCCUCUAU G CCAUU | 7016 | AAUGC UGAUG GCAUGCACUAUGC GCC AUAGACCGUA | 16356 |
| 1353 | UUCCUCCCCC G CAUCA | 7017 | UGAUG UGAUC GCAUGCACUAUGC GCC CCGCCAGGAA | 16357 |
| 1391 | CAGCAAGAGU G CGCCA | 7018 | UGCCG UGAUG GCAUGCACUAUGC GCG ACUCUUCCUC | 16358 |
| 1393 | GGAAGAGUCC G CCAAC | 7019 | CUUCC UGAUG GCAUGCACUAUGC GCC GCACUCUUCC | 16359 |
| 1399 | GUGCGCCAAC G AGCCC | 7020 | GCGCU UGAUG GCAUGCACUAUGC GCG CUUGCCCCAC | 16360 |
| 1422 | CUCUCUCAGU G ACAAA | 7021 | UUUCU UGAUC GCAUGCACUAUGC GCG ACUGACACAC | 16361 |
| 1441 | AUACCCUUGU G AAGAA | 7022 | UUCUU UGAUC GCAUGCACUAUGC GCG ACAAGCGUAU | 16362 |
| 1486 | AAAUAAAAUU G AACUU | 7023 | AACUU UGAUG GCAUGCACUAUGC GCG AAUUUUAUUU | 16363 |
| 1507 | AAAUCAAUUU G CUCUA | 7024 | UACAG UGAUG GCAUGCACUAUGC GCC AAAUUCAUUU | 16364 |
| 1516 | UGCUCUAAUU G AACGA | 7025 | UCCUU UGAUG GCAUGCACUAUGC GCC AAUUAGAGCA | 16365 |
| 1585 | GUACAAAUCU G AAGCC | 7026 | CCCUU UGAUC GCAUCCACUAUGC CCG ACAUUUGUAC | 16366 |
| 1620 | GACACACCCU G AUCUC | 7027 | CACAU UGAUG GCAUGCACUAUGC GCG ACCCUCUCUC | 16367 |
| 1635 | CCUUCCACCU G ACCAC | 7028 | CUCCU UGAUG GCAUGCACUAUGC GCG ACCUCCAACC | 16368 |
| 1648 | CACCCCUCCU G AAAUU | 7029 | AAUUU UGAUG GCAUGCACUAUGC GCG ACCACCCCUC | 16369 |
| 1659 | AAAUUACUUU G CAACC | 7030 | CCUUC UGAUG GCAUGCACUAUGC GCG AAACUAAUUU | 16370 |
| 1666 | UUUCCAACCU G ACAUC | 7031 | CAUCU UGAUG GCAUGCACUAUGC GCC ACCUUCCAAA | 16371 |
| 1671 | AACCUCACAU G CACCC | 7032 | CCCUC UGAUG GCAUGCACUAUGC GCG AUCUCACCUU | 16372 |
| 1681 | CCACCCCACU G ACCAC | 7033 | CUCCU UGAUG GCAUGCACUAUGC GCG ACUGCCCUCC | 16373 |
| 1706 | UCUUUCUCCU G CACUC | 7034 | CACUC UGAUG GCAUGCACUAUGC GCG ACCACAAACA | 16374 |
| 1711 | CUCCUCCACU G CACAC | 7035 | CUCUC UGAUG GCAUGCACUAUGC GCG ACUCCACCAC | 16375 |
| 1729 | AUCUACCUUU G ACAAC | 7036 | CUUCU UGAUG GCAUGCACUAUGC GCG AAACCUACAU | 16376 |
| 1767 | CACACCCUCU G CCAAU | 7037 | AUUCC UGAUG GCAUGCACUAUGC GCG ACACCCUCUC | 16377 |
| 1788 | UCCCACACUU G CCCAC | 7038 | CUCCC UGAUG GCAUGCACUAUGC GCG AACUCUCCCA | 16378 |
| 1802 | ACACCUCUUU G CAACA | 7039 | UCUUC UGAUG GCAUGCACUAUGC GCG AAACACCUCU | 16379 |
| 1830 | UUUCCAAAUU G AAUCC | 7040 | CCAUU UGAUG GCAUGCACUAUGC GCG AAUUUCCAAA | 16380 |
| 1834 | CAAAUUCAAU G CCACC | 7041 | CCUCC UGAUG GCAUGCACUAUGC GCG AUUCAAUUUC | 16381 |
| 1861 | UACCACAAAU G ACAUU | 7042 | AAUCU UGAUG GCAUGCACUAUGC GCG AUUUCUCCUA | 16382 |
| 1869 | AUCACAUUUU G AUCAU | 7043 | AUCAU UGAUG GCAUGCACUAUGC GCG AAAAUCUCAU | 16383 |
| 1888 | CCUUAACAAU G CAUCC | 7044 | CCAUC UGAUG GCAUGCACUAUGC GCG AUUCUUAACC | 16384 |
| 1896 | AUCCAUCCUU G CACCA | 7045 | UCCUC UGAUG GCAUGCACUAUGC GCG AACCAUCCAU | 16385 |
| 1919 | CACUAUCUCU G CCUUC | 7046 | CAACC UGAUG GCAUGCACUAUGC GCG ACACAUACUC | 16386 |
| 1924 | UCUCUCCCUU G CUCAA | 7047 | UUCAC UGAUG GCAUGCACUAUGC GCG AACCCACACA | 16387 |
| 1955 | AAAACACAUU G CCUCC | 7048 | CCACC UGAUG GCAUGCACUAUGC GCG AAUCUCUUUU | 16388 |
| 1998 | UCCCACCCAC G AUCAC | 7049 | CUCAU UGAUG GCAUGCACUAUGC GCG CUCCCUCCCA | 16389 |
| 2025 | ACAAUCACAC G ACAAC | 7050 | CUUCU UGAUG GCAUGCACUAUGC GCG CUCUCAUUCU | 16390 |
| 2047 | CCAAACCAUC G AACUC | 7051 | CACUU UGAUG GCAUGCACUAUGC GCG CAUCCUUUCC | 16391 |
| 2057 | CAACUCUCAU G CACCC | 7052 | CCCUC UGAUG GCAUGCACUAUGC GCG AUCACACUUC | 16392 |
| 2107 | UAAACAUAAU G ACACC | 7053 | CCUCU UGAUG GCAUGCACUAUGC GCG AUUAUCUUUA | 16393 |
| 2139 | CCAUUCUAUU G AACCA | 7054 | UCCUU UGAUG GCAUGCACUAUGC GCG AAUACAAUCC | 16394 |
| 2168 | CUCACUAUCC G CACAC | 7055 | CUCUC UGAUG GCAUGCACUAUGC GCG CCAUACUCAC | 16395 |
| 2175 | UCCCCACACU G ACCAA | 7056 | UUCCU UGAUG GCAUGCACUAUGC GCG ACUCUCCCCA | 16396 |
| 2188 | CAACCACCAC G AACCC | 7057 | CCCUU UGAUG GCAUGCACUAUGC GCG CUCCUCCUUC | 16397 |
| 2204 | CUCUACACCU G CCAGC | 7058 | GCUGG UGAUG GCAUGCACUAUGC GGG AGGUGUACAC | 16398 |
| 2213 | UGCCAGGCAU G CACUG | 7059 | CACUG UGAUG GCAUGCACUAUGC GCG AUGCCUCGCA | 16399 |
| 2230 | UCUUGGCUGU G CAAAA | 7060 | UUUUC UGAUC GCAUGCACUAUGC GCC ACAGCCAAGA | 16400 |
| 2263 | AAUAGAAGGU G CGCAG | 7061 | CUGGG UGAUG CCAUGCACUAUGC CCG ACCUUCUAUU | 16401 |
| 2277 | AGGAAAAGAC G AACUU | 7062 | AAGUU UGAUG CCAUGCACUAUGC GGG CUCUUUUCCU | 16402 |
| 2307 | UAGUAGGCAC G ACCGU | 7063 | ACCGU UGAUG GCAUGCACUAUGC GCG CUCCCUACUA | 16403 |
| 2313 | CCACGACGCU G AUUCC | 7064 | GCAAU UGAUC GCAUGCACUAUGC GGC ACCCUCGUGC | 16404 |
| 2317 | GACGGUGAUU G CCAUC | 7065 | CAUCC UGAUC GCAUGCACUAUGC GCG AAUCACCGUC | 16405 |
| 2385 | GACCCGAACU G AAGAC | 7066 | CUCUU UGAUG GCAUGCACUAUGC GCG ACUUCCCCUC | 16406 |
| 2422 | GGAUCCACAU G AACUC | 7067 | CACUU UGAUC GCAUGCACUAUGC GCG AUCUGCAUCC | 16407 |
| 2437 | CCCAUUCCAU G AACAU | 7068 | AUCUU UGAUC GCAUGCACUAUGC GCG AUCCAAUGGC | 16408 |
| 2446 | UGAACAUUCU G AACGA | 7069 | UCGUU UGAUG GCAUGCACUAUGC GCG ACAAUGUUCA | 16409 |
| 2450 | CAUUCUGAAC G ACUCC | 7070 | GCACU UGAUG GCAUGCACUAUGC GCG GUUCACAAUC | 16410 |
| 2454 | CUCAACCACU G CCUUA | 7071 | UAAGG UGAUG GCAUGCACUAUGC GCC ACUCCUCCAC | 16411 |
| 2461 | ACUCCCUUAU G AUGCC | 7072 | CCCAU UGAUG GCAUGCACUAUGC GCC AUAACGCACU | 16412 |
| 2464 | GCCUUAUCAU G CCACC | 7073 | GGUCG UGAUG GCAUGCACUAUGC GCG AUCAUAAGGC | 16413 |
| 2496 | CACACCCCCU G AACCU | 7074 | ACCUU UGAUG GCAUGCACUAUGC GCG ACCCCCUCUC | 16414 |
| 2524 | UCCCCCUCCU G CCUUU | 7075 | AAACC UGAUG GCAUGCACUAUGC GCG ACCACCCCCA | 16415 |
| 2542 | CCAACACAUU G AACCA | 7076 | UCCUC UGAUG GCAUGCACUAUGC GCG AAUCUCUUCC | 16416 |
| 2551 | UCAACCACAU G CCUUU | 7077 | AAACC UGAUG GCAUGCACUAUGC GCG AUCUCCUUCA | 16417 |
| 2563 | CUUUCCAAUU G ACAAC | 7078 | CUUCU UGAUG GCAUGCACUAUGC GCG AAUUCCAAAC | 16418 |
| 2579 | ACACCAACUU G CACCA | 7079 | UCCUC UGAUG GCAUGCACUAUGC GCG AACUUCCUCU | 16419 |
| 2604 | UCAAAAUCUU G AAACA | 7080 | UCUUU UGAUG GCAUGCACUAUGC GCG AACAUUUUCA | 16420 |
| 2626 | AACACACACU G ACCAU | 7081 | AUCCU UGAUG GCAUGCACUAUGC GCG ACUCUCUCUC | 16421 |
| 2633 | ACUCACCAUC G ACCUC | 7082 | CACCU UGAUC GCAUGCACUAUGC GCG CAUCCUCACU | 16422 |
| 2647 | UCUGAUGUCU G AACUC | 7083 | CACUC UGAUC GCAUGCACUAUGC GCG ACACAUCACA | 16423 |
| 2704 | CCUUCUACCU G CCUCU | 7084 | ACACC UGAUG GCAUGCACUAUGC GCG ACCUACAACC | 16424 |
| 2736 | CACUGAUGCU G AUUCU | 7085 | ACAAU UGAUG GCAUGCACUAUGC GCG ACCAUGACUC | 16425 |
| 2750 | CUCCAAUUCU G CAAAU | 7086 | AUUUC UGAUG GCAUGCACUAUGC GCG ACAAUUCCAC | 16426 |
| 2778 | CCACUUACCU G ACCAC | 7087 | CUCCU UGAUG GCAUGCACUAUGC GCG ACCUAACUCC | 16427 |
| 2794 | CAACACAAAU G AAUUU | 7088 | AAAUU UGAUG GCAUGCACUAUGC GCG AUUUCUCCUC | 16428 |
| 2825 | AAACCCCCAC G AUUCC | 7089 | CCAAU UGAUG GCAUGCACUAUGC GCG CUCCCCCUUU | 16429 |
| 2871 | CUCUCCAUCU G AAACC | 7090 | GCHUG UGAUG GCAUGCACUAUGC GCG ACAUCCACAC | 16430 |
| 2879 | CUCAAACCCC G CUUCC | 7091 | CCAAC UGAUG GCAUGCACUAUGC GCG CGCCUUCCAC | 16431 |
| 2947 | CUCCCUCACU G AUCUA | 7092 | UACAU UGAUG GCAUGCACUAUGC GCG ACUCACCCAC | 16432 |
| 2971 | CCAACCUCCU G AACAU | 7093 | AUCUG UGAUG GCAUGCACUAUGC GCG ACCACCUUCG | 16433 |
| 2994 | ACCACUUCCU G ACCUU | 7094 | AACCU UGAUG GCAUGCACUAUGC GCG ACCAACUCCU | 16434 |
| 3057 | UUUUGGCAUC G CCAAA | 7095 | UUUGC UGAUG GCAUGCACUAUGC GCG CAUGCCAACA | 16435 |
| 3059 | UUCCCAUCCC G AAACU | 7096 | ACUUG UGAUG GCAUGCACUAUGC GCG CCCAUCCCAA | 16436 |
| 3089 | CUCCGCCGAC G AAAUA | 7097 | UAUUU UGAUG GCAUGCACUAUGC GCG CUCGCCGCAC | 16437 |
| 3130 | UAAAAUCUCU G ACUUU | 7098 | AAACU UGAUG GCAUGCACUAUGC GCG ACACAUCUGA | 16438 |
| 3187 | AAAACCACAU G CUCCG | 7099 | CGCAC UGAUG GCAUGCACUAUGC GCG AUCUCCUUUU | 16439 |
| 3191 | CCACAUCCUC G GCUCG | 7100 | CCACC UGAUG GCAUGCACUAUGC GCG CACCAUCUCC | 16440 |
| 3201 | CCCUCCCUUU G AAAUC | 7101 | CAUUU UGAUG GCAUGCACUAUGC GCG AAACCCACCC | 16441 |
| 3229 | AACAAUUUUU G ACACA | 7102 | UCUCU UGAUG GCAUGCACUAUGC GCG AAAAUUUCUU | 16442 |
| 3253 | AAUCCACACU G ACCUC | 7103 | CACCU UGAUG GCAUGCACUAUGC GCG ACUCUCCAUC | 16443 |
| 3276 | UUCCUCUUUG G CUCUC | 7104 | CACAC UGAUG GCAUGCACUAUGC GCG AAAACACCAA | 16444 |
| 3301 | UUCCUUACCU G CUUCU | 7105 | ACAAC UGAUG GCAUGCACUAUGC GCG ACCUAACCAA | 16445 |
| 3328 | CCUAAACAUU G AUGAA | 7106 | UUCAU UGAUG GCAUGCACUAUGC GCG AAUCUUUACC | 16446 |
| 3331 | AAACAUUCAU G AACAA | 7107 | UUCUU UGAUG GCAUGCACUAUGC GCG AUCAAUCUUU | 16447 |
| 3347 | UUUUAUACGC G AUUGA | 7108 | UCAAU UGAUG GCAUGCACUAUGC GCG GCCUACAAAA | 16448 |
| 3351 | GUAGGCCAUU G AAAGA | 7109 | UCUUU UGAUG GCAUGCACUAUGC GCG AAUCGCCUAC | 16449 |
| 3369 | GAACUAGAAU U AGGUC | 7110 | GCCCU UGAUG GCAUGCACUAUGC GCG AUUCUAGUUC | 16450 |
| 3379 | GAGGGCCCCU G AUUAU | 7111 | AUAAU UGAUG GCAUGCACUAUGC GCG AGGGGCCCUC | 16451 |
| 3411 | ACCAGACCAU U CUGGA | 7112 | UCCAG UGAUG GCAUGCACUAUGC GCG AUGGUCUGGG | 16452 |
| 3419 | AUGCUGGACU U CUGUC | 5872 | GCCAG UGAUG GCAUGCACUAUGC GCG AGUCCAGCAU | 15212 |
| 3486 | GAAAUCUCUU G CAAGC | 7113 | GCUUG UGAUG GCAUGCACUAUGC GCG AAGAGAUUUC | 16453 |
| 3496 | GCAAGCUAAU U CUCAG | 7114 | CUGAG UGAUG GCAUGCACUAUGC GCG AUUAGCUUGC | 16454 |
| 3531 | UUGUUCUUCC G AUAUC | 7115 | CAUAU UGAUG GCAUGCACUAUGC GCG GCAAGAACAA | 16455 |
| 3546 | CACACACUUU G AGCAU | 7116 | AUCCU UGAUG GCAUGCACUAUGC GCG AAAGUCUCUG | 16456 |
| 3576 | CACUCUCUCU G CCUAC | 7117 | GUACG UGAUG GCAUGCACUAUGC GCG AGAGACACUC | 16457 |
| 3619 | GGAACUAUGU G ACCCC | 7118 | GGGCU UGAUG GCAUGCACUAUGC GCG ACAUACUUCC | 16458 |
| 3637 | AUUCCAUUAU G ACAAC | 7119 | GUUGU UGAUC GCAUGCACUAUGC GCC AUAAUGCAAU | 16459 |
| 3666 | CUCACUAUCU G CAGAA | 7120 | UUCUG UGAUG CCAUGCACUAUGC GCG ACAUACUCAC | 16460 |
| 3680 | AACAGUAACC G AAACA | 7121 | UCUUU UGAUG CCAUGCACUAUGC GCG CCUUACUGUU | 16461 |
| 3696 | GCCCGCCUGU G AGUCU | 7122 | ACACU UGAUG CCAUGCACUAUGC GCG ACACCCCGGC | 16462 |
| 3712 | AAAAACAUUU G AAGAU | 7123 | AUCUU UGAUG GCAUGCACUAUGC CCG AAAUGUUUUU | 16463 |
| 3757 | AAUCCCAGAU G ACAAC | 7124 | GUUGU UGAUG GCAUGCACUAUGC GCG AUCUGCGAUU | 16464 |
| 3787 | UAUGGUUCUU G CCUCA | 7125 | UCAGC UGAUG GCAUGCACUAUGC GCG AAGAACCAUA | 16465 |
| 3801 | CAGAAGAGCU G AAAAC | 7126 | GUUUU UGAUG GCAUGCACUAUGC CCG AGCUCUUCUG | 16466 |
| 3852 | GUCGAAUGGU G CCCAG | 7127 | CUCGC UGAUG GCAUGCACUAUGC GCG ACCAUUCCAC | 16467 |
| 3883 | UGUGCCAUCU G AAGCC | 7128 | GCCUU UGAUG GCAUGCACUAUGC CCG AGACUCCACA | 16468 |
| 3928 | AUAUCACUCC G AUCAC | 7129 | CUCAU UGAUC GCAUGCACUAUGC GCG CCACUCAUAU | 16469 |
| 3931 | UCACUCCGAU G ACACA | 7130 | UCUCU UGAUG GCAUGCACUAUGC GCG AUCGGAGUGA | 16470 |
| 3958 | CUACUCCAUG G ACCAA | 7131 | UUCCU UGAUG GCAUGCACUAUGC GCG ACUCCACUAC | 16471 |
| 3981 | UUUUAAAGCU G AUACA | 7132 | UCUAU UGAUG GCAUGCACUAUGC GCG AGCUUUAAAA | 16472 |
| 3996 | ACAUUCGAGU G CAAAC | 7133 | GUUUC UGAUG GCAUGCACUAUGC GCG ACUCCAAUCU | 16473 |
| 4030 | UCUCCACCCU G ACACC | 7134 | CCUCU UGAUG GCAUGCACUAUGC GCG ACUCUCCACA | 16474 |
| 4047 | CCACCACACU G ACCUC | 7135 | CACCU UGAUG GCAUGCACUAUGC GCG ACUCUCCUCC | 16475 |
| 4103 | CACAUCACAU G ACAUC | 7136 | CCUCU UGAUG GCAUGCACUAUGC GCG AUCUGAUGUC | 16476 |
| 4112 | UCACACCUCU G CUCAC | 7137 | CUCAC UGAUG GCAUGCACUAUGC GCG ACACCUCUCA | 16477 |
| 4123 | CUCACAUUUU G AACUC | 7138 | CACUU UGAUG GCAUGCACUAUGC GCG AAAAUCUCAC | 16478 |
| 4157 | CCAACUACCC G CAUUU | 7139 | AAAUC UGAUG GCAUGCACUAUGC GCG CCCUACUUCC | 16479 |
| 4163 | ACCCCCAUUU G AUUUU | 7140 | AAAAU UGAUG GCAUGCACUAUGC GCG AAAUCCCCCU | 16480 |
| 4175 | UUUUCAUUUC G ACAAC | 7141 | CUUCU UGAUG GCAUGCACUAUGC GCG CAAAUCAAAA | 16481 |
| 4200 | ACCUCCCACU G CACCC | 7142 | CCCUC UGAUG GCAUGCACUAUGC GCG ACUCCCACCU | 16482 |

TABLE XVIII
Human FLT DNAzyme and Substrate Sequence

| | | Seq ID | | Seq ID |
|---|---|---|---|---|
| Pos | Substrate | No | DNAzyme | No |
| 17 | UCCUCUCG G CUCCUCCC | 7143 | GGGAGGAG GGCTAGCTACAACGA CGAGAGGA | 16483 |
| 28 | CCUCCCCG G CAGCGGCG | 7144 | CGCCGCTG GGCTAGCTACAACGA CGGGGAGG | 16484 |
| 31 | CCCCGGCA G CGGCGGCG | 7145 | CGCCGCCG GGCTAGCTACAACGA TGCCGGGG | 16485 |
| 34 | GGGCAGCG G CGGCGGCU | 7146 | AGCCGCCC GGCTAGCTACAACGA CGCTGCCG | 16486 |
| 37 | CAGCGGCG G CGGCUCGG | 7147 | CGGAGCCC GGCTAGCTACAACGA CGCCGCTG | 16487 |
| 40 | CGGCGGCG G CUCGGAGC | 7148 | GCTCCCAC GGCTAGCTACAACGA CGCCGCCG | 16488 |
| 47 | GGCUCGGA G CGGGCUCC | 7149 | GGAGCCCG GGCTAGCTACAACGA TCCGAGCC | 16489 |
| 51 | CGGAGCGG G CUCCGGGG | 7150 | CCCCGGAG GGCTAGCTACAACGA CCGCTCCG | 16490 |
| 59 | GCUCCGGG G CUCGGGUG | 7151 | CACCCGAG GGCTAGCTACAACGA CCCGGAGC | 16491 |
| 65 | GGCCUCGG G UGCAGCGG | 7152 | CCGCTGCA GGCTAGCTACAACGA CCGAGCCC | 16492 |
| 67 | GCUCGGGU G CAGCGGCC | 7153 | GGCCGCTG GGCTAGCTACAACGA ACCCGAGC | 16493 |
| 70 | CGGGUGCA G CGGCCAGC | 7154 | GCTGGCCG GGCTAGCTACAACGA TGCACCCG | 16494 |
| 73 | GUGCAGCG G CCAGCGGG | 7155 | CCCGCTGG GGCTAGCTACAACGA CGCTGCAC | 16495 |
| 77 | AGCGGCCA G CCGGCCUG | 7156 | CAGGCCCC GGCTAGCTACAACGA TGCCCGCT | 16496 |
| 81 | GCCAGCGG G CCUGCCGC | 7157 | CCGCCACC GGCTAGCTACAACGA CCGCTGGC | 16497 |
| 86 | CGGCCCUG G CCCCGACG | 7158 | CCTCGCCC GGCTAGCTACAACGA CAGGCCCG | 16498 |
| 89 | GCCUGGCG G CCAGCAUU | 7159 | AATCCTCG GGCTAGCTACAACGA CGCCAGGC | 16499 |
| 95 | CGGCGAGG A UUACCCGC | 7160 | CCCCGTAA GGCTAGCTACAACGA CCTCGCCG | 16500 |
| 98 | CGAGGAUU A CCCGCGGA | 10 | TCCCCGCG GGCTAGCTACAACGA AATCCTCG | 16501 |
| 108 | CCGGGGAA G UGGUUCUC | 7161 | CACAACCA GGCTAGCTACAACGA TTCCCCGG | 16502 |
| 111 | GGGAAGUG G UUGUCUCC | 7162 | GGAGACAA GGCTAGCTACAACGA CACTTCCC | 16503 |
| 114 | AAGUGGUU G UCUCCUGC | 7163 | CCAGGAGA GGCTAGCTACAACGA AACCACTT | 16504 |
| 122 | GUCUCCUG G CUGGAGCC | 7164 | GCCTCCAG GGCTAGCTACAACGA CAGGAGAC | 16505 |
| 128 | UGGCUGGA G CCGCGAGA | 7165 | TCTCGCGG GGCTAGCTACAACGA TCCAGCCA | 16506 |
| 131 | CUGGAGCC G CGACACCG | 7166 | CCGTCTCC GGCTAGCTACAACGA GCCTCCAG | 16507 |
| 136 | GCCGCCAG A CCCGCCCU | 7167 | ACCCCCCG GGCTAGCTACAACGA CTCGCGGC | 16508 |
| 140 | CGAGACGG G CCCUCAGG | 7168 | CCTCACCC GGCTAGCTACAACGA CCGTCTCG | 16509 |
| 142 | AGACGGGC G CUCACCGC | 7169 | CCCCTGAC GGCTAGCTACAACGA GCCCGTCT | 16510 |
| 149 | CGCUCAGG G CCCCCCCC | 7170 | GCCCCGCG GGCTAGCTACAACGA CCTGAGCG | 16511 |
| 151 | CUCAGGGC G CCCCCCCC | 7171 | CGGCCCCG GGCTAGCTACAACGA GCCCTGAG | 16512 |
| 156 | GGCGCGGG G CCCCCCCC | 7172 | GCCGCCGG GGCTAGCTACAACGA CCCGCGCC | 16513 |
| 160 | CGGGGCCG G CCCCCCCC | 7173 | CGCCGCCG GGCTAGCTACAACGA CGGCCCCG | 16514 |
| 163 | GGCCGGCG G CCCCCAAC | 7174 | GTTGCCCG GGCTAGCTACAACGA CGCCGGCC | 16515 |
| 166 | CGGCGGCG G CCAACCAC | 7175 | CTCGTTCG GGCTAGCTACAACGA CGCCGCCG | 16516 |
| 170 | GGCGGCGA A CCACACCA | 7176 | TCCTCTCG GGCTAGCTACAACGA TCGCCGCC | 16517 |
| 178 | ACGAGAGG A CCCACUCU | 7177 | AGAGTCCG GGCTAGCTACAACGA CCTCTCGT | 16518 |
| 182 | GAGGACGG A CUCUCCCC | 7178 | CGCCAGAG GGCTAGCTACAACGA CCGTCCTC | 16519 |
| 188 | GGACUCUG G CCCCCCCC | 7179 | CCCGGCCG GGCTAGCTACAACGA CAGAGTCC | 16520 |
| 191 | CUCUGGCG G CCCCCUCC | 7180 | CGACCCGG GGCTAGCTACAACGA CGCCAGAC | 16521 |
| 196 | GCGGCCGG G UCGUUGGC | 7181 | GCCAACGA GGCTAGCTACAACGA CCGGCCGC | 16522 |
| 199 | GCCGGGUC G UUGGCCGG | 7182 | CCGGCCAA GGCTAGCTACAACGA GACCCGGC | 16523 |
| 203 | GGUCGUUG G CCGGGGGA | 7183 | TCCCCCGG GGCTAGCTACAACGA CAACGACC | 16524 |
| 212 | CCGGGGGA G CGCGGGCA | 7184 | TGCCCGCG GGCTAGCTACAACGA TCCCCCGG | 16525 |
| 214 | GGGGGAGC G CGGGCACC | 7185 | GGTGCCCG GGCTAGCTACAACGA GCTCCCCC | 16526 |
| 218 | GAGCGCGG G CACCGGGC | 7186 | GCCCGGTG GGCTAGCTACAACGA CCGCGCTC | 16527 |
| 220 | GCGCGGGC A CCGGGCGA | 4191 | TCGCCCGG GGCTAGCTACAACGA GCCCGCGC | 16528 |
| 225 | GGCACCGG G CGAGCAGG | 7187 | CCTCCTCC GGCTAGCTACAACGA CCGGTGCC | 16529 |
| 229 | CCCGCCGA G CAGGCCGC | 7188 | GCGGCCTG GGCTAGCTACAACCA TCGCCCGG | 16530 |
| 233 | GCGACCAG G CCGCGUCG | 7189 | CGACGCCC GGCTAGCTACAACGA CTGCTCGC | 16531 |
| 236 | ACCAGGGC G CGUCGCGC | 7190 | GCGCGACG GGCTAGCTACAACGA GGCCTGCT | 16532 |
| 238 | CAGGCCGC G UCGCGCUC | 7191 | GAGCGCGA GGCTAGCTACAACGA GCGGCCTG | 16533 |
| 241 | GCCGCGUC G CGCUCACC | 7192 | GGTGAGCG GGCTAGCTACAACGA CACGCGGC | 16534 |
| 243 | CGCCUCGC G CUCACCAU | 7193 | ATCGTGAG GGCTAGCTACAACGA CCCACCCG | 16535 |
| 247 | UCGCGCUC A CCAUCGUC | 4196 | GACCATGG GGCTAGCTACAACGA GAGCGCGA | 16536 |
| 250 | CGCUCACC A UGCUCACC | 4198 | GCTCACCA GGCTAGCTACAACGA GGTCACCC | 16537 |
| 253 | UCACCAUG G UCACCUAC | 7194 | GTAGCTGA GGCTAGCTACAACGA CATCGTCA | 16538 |
| 257 | CAUGGUCA G CUACUGGG | 7195 | CCCAGTAG GGCTAGCTACAACGA TGACCATC | 16539 |
| 260 | GGUCAGCU A CUGGGACA | 21 | TGTCCCAC GGCTAGCTACAACGA ACCTGACC | 16540 |
| 266 | CUACUCGG A CACCGGGG | 7196 | CCCCCGTG GGCTAGCTACAACGA CCCACTAG | 16541 |
| 268 | ACUCGGAC A CCGGGGUC | 4202 | CACCCCCG GGCTAGCTACAACGA GTCCCAGT | 16542 |
| 274 | ACACCGGC G UCCUGCUG | 7197 | CAGCAGGA GGCTAGCTACAACGA CCCGCTGT | 16543 |
| 279 | GCGGUCCU G CUGUGCCC | 7198 | GCGCACAG GGCTAGCTACAACGA ACGACCCC | 16544 |
| 282 | GUCCUGCU G UGCGCGCU | 7199 | AGCGCGCA GGCTAGCTACAACGA ACCAGGAC | 16545 |
| 284 | CCUGCUGU G CGCCCUGC | 7200 | CCACCCCG GGCTAGCTACAACGA ACAGCACG | 16546 |
| 286 | UGCUGUCC G CGCUGCUC | 7201 | GACCAGCC GGCTAGCTACAACGA GCACAGCA | 16547 |
| 288 | CUGUCCGC G CUGCUCAC | 7202 | CTCAGCAG GGCTAGCTACAACGA GCCCACAG | 16548 |
| 291 | UCCCCCCU G CUCAGCUG | 7203 | CAGCTGAG GGCTAGCTACAACGA AGCGCGCA | 16549 |
| 296 | GCUGCUCA G CUGUCUGC | 7204 | GCACACAG GGCTAGCTACAACGA TGAGCAGC | 16550 |
| 299 | GCUCAGCU G UCUGCUUC | 7205 | GAACCAGA GGCTAGCTACAACGA AGCTGACC | 16551 |
| 303 | ACCUGUCU G CUUCUCAC | 7206 | CTCACAAC GGCTAGCTACAACGA ACACACCT | 16552 |
| 310 | UGCUUCUC A CACGAUCU | 4214 | AGATCCTG GGCTAGCTACAACGA GACAACCA | 16553 |
| 315 | CUCACAGC A UCUAGUUC | 7207 | CAACTAGA GGCTAGCTACAACGA CCTCTCAG | 16554 |
| 320 | ACGAUCUA G UUCACCUU | 7208 | AACCTGAA GGCTAGCTACAACGA TAGATCCT | 16555 |
| 326 | UAGUUCAG G UUCAAAAU | 7209 | ATTTTCAA GGCTAGCTACAACGA CTCAACTA | 16556 |
| 333 | GGUUCAAA A UUAAAAAG | 7210 | TCTTTTAA GGCTAGCTACAACGA TTTGAACC | 16557 |
| 341 | AUUAAAAC A UCCUCAAC | 7211 | CTTCAGCA GGCTAGCTACAACGA CTTTTAAT | 16558 |
| 348 | CAUCCUCA A CUCAGUUU | 7212 | AAACTCAG GGCTAGCTACAACGA TCAGGATC | 16559 |
| 353 | UCAACUGA G UUUAAAAG | 7213 | CTTTTAAA GGCTAGCTACAACGA TCACTTCA | 16560 |
| 362 | UUUAAAAC G CACCCAGC | 7214 | CCTCGGTG GGCTAGCTACAACGA CTTTTAAA | 16561 |
| 364 | UAAAACGC A CCCAGCAC | 4222 | CTGCTGGG GGCTAGCTACAACGA GCCTTTTA | 16562 |
| 369 | CGCACCCA G CACAUCAC | 7215 | ATCATCTC GGCTAGCTACAACGA TGCCTCCC | 16563 |
| 371 | CACCCAGC A CAUGAUGC | 4226 | GCATGATC GGCTAGCTACAACGA CCTCCCTC | 16564 |
| 373 | CCCAGCAC A UGAUGCAA | 4227 | TTCCATCA GGCTAGCTACAACGA CTCCTGCC | 16565 |
| 376 | ACCACAUC A UCCAACCA | 4228 | TCCTTCCA GGCTAGCTACAACGA GATCTGCT | 16566 |
| 378 | CACAUCAU G CAACCACC | 7216 | CCTCCTTC GGCTAGCTACAACGA ATCATCTC | 16567 |
| 382 | UGAUGCAA G CACCCCAC | 7217 | CTCCCCTG GGCTAGCTACAACGA TTCCATCA | 16568 |
| 386 | GCAAGCAG G CCACACAC | 7218 | CTCTCTCC GGCTAGCTACAACGA CTGCTTCC | 16569 |
| 391 | CAGCCCAC A CACUCCAC | 7219 | ATCCAGTC GGCTAGCTACAACGA CTGGCCTG | 16570 |
| 393 | GGCCACAC A CUCCAUCU | 4233 | AGATGCAG GGCTAGCTACAACGA GTCTGCCC | 16571 |
| 396 | CAGACACU G CAUCUCCA | 7220 | TGGACATG GGCTAGCTACAACGA AGTGTCTC | 16572 |
| 398 | GACACUGC A UCUCCAAU | 4235 | ATTGCACA GGCTAGCTACAACGA CCACTCTC | 16573 |
| 405 | CAUCUCCA A UGCAGCGG | 7221 | CCCCTCCA GGCTAGCTACAACGA TCCACATG | 16574 |
| 407 | UCUCCAAU G CAGGCCCC | 7222 | CCCCCCTC GGCTAGCTACAACGA ATTCCAGA | 16575 |
| 418 | GGGGGGAA G CACCCCAU | 7223 | ATCCCCTC GGCTAGCTACAACGA TTCCCCCC | 16576 |
| 421 | GGGAAGCA G CCCAUAAA | 7224 | TTTATGGG GGCTAGCTACAACGA TGCTTCCC | 16577 |
| 425 | AGCAGCCC A UAAAUGGU | 4243 | ACCATTTA GGCTAGCTACAACGA GGGCTGCT | 16578 |
| 429 | GCCCAUAA A UGGUCUUU | 7225 | AAAGACCA GGCTAGCTACAACGA TTATGGGC | 16579 |
| 432 | CAUAAAUG G UCUUUGCC | 7226 | GGCAAAGA GGCTAGCTACAACGA CATTTATG | 16580 |
| 438 | UGGUCUUU G CCUGAAAU | 7227 | ATTTCAGG GGCTAGCTACAACGA AAAGACCA | 16581 |
| 445 | UGCCUGAA A UGCUGAGU | 7228 | ACTCACCA GGCTAGCTACAACGA TTCAGGCA | 16582 |
| 448 | CUGAAAUG G UGAGUAAG | 7229 | CTTACTCA GGCTAGCTACAACGA CATTTCAG | 16583 |
| 452 | AAUGGUGA G UAAGGAAA | 7230 | TTTCCTTA GGCTAGCTACAACGA TCACCATT | 16584 |
| 461 | UAAGGAAA G CGAAACGC | 7231 | GCCTTTCG GGCTAGCTACAACGA TTTCCTTA | 16585 |
| 468 | AGCGAAAG C CUCAGCAU | 7232 | ATGCTCAG GGCTAGCTACAACGA CTTTCCCT | 16586 |
| 473 | AAGGCUCA G CAUAACUA | 7233 | TAGTTATG GGCTAGCTACAACGA TCAGCCTT | 16587 |
| 475 | GGCUGAGC A UAACUAAA | 4248 | TTTAGTTA GGCTAGCTACAACGA GCTCAGCC | 16588 |
| 478 | UGAGCAUA A CUAAAUCU | 7234 | AGATTTAC GGCTAGCTACAACGA TATGCTCA | 16589 |
| 483 | AUAACUAA A UCUGCCUC | 7235 | CAGGCACA GGCTAGCTACAACGA TTAGTTAT | 16590 |
| 487 | CUAAAUCU C CCUGUGGA | 7236 | TCCACAGC GGCTAGCTACAACGA AGATTTAG | 16591 |
| 491 | AUCUCCCU C UGCAAGAA | 7237 | TTCTTCCA GGCTAGCTACAACGA AGGCAGAT | 16592 |
| 500 | UGGAACAA A UCCCAAAC | 7238 | GTTTGCCA GGCTAGCTACAACGA TTCTTCCA | 16593 |
| 503 | AAGAAAUG G CAAACAAU | 7239 | ATTCTTTG GGCTAGCTACAACGA CATTTCTT | 16594 |
| 507 | AAUCGCAA A CAAUUCUC | 7240 | CAGAATTC GGCTAGCTACAACGA TTGCCATT | 16595 |
| 510 | CGCAAACA A UUCUCCAC | 7241 | CTCCACAA GGCTAGCTACAACGA TCTTTGCC | 16596 |
| 515 | ACAAUUCU G CACUACUU | 7242 | AAGTACTC GGCTAGCTACAACGA AGAATTGT | 16597 |
| 518 | AUUCUGCA C UACUUUAA | 7243 | TTAAACTA GGCTAGCTACAACGA TGCACAAT | 16598 |
| 520 | UCUGCACU A CUUUAACC | 53 | GCTTAAAC GGCTAGCTACAACGA ACTCCAGA | 16599 |
| 526 | GUACUUUA A CCUUGAAC | 7244 | GTTCAAGG GGCTAGCTACAACGA TAAACTAC | 16600 |
| 533 | AACCUUCA A CACACCUC | 7245 | CAGCTGTG GGCTAGCTACAACGA TCAAGCTT | 16601 |
| 535 | CCUUGAAC A CAGCUCAA | 4260 | TTCAGCTC GGCTAGCTACAACGA GTTCAAGG | 16602 |
| 538 | UCAACACA C CUCAAGCA | 7246 | TCCTTCAG GGCTAGCTACAACGA TCTCTTCA | 16603 |
| 544 | CACCUCAA C CAAACCAC | 7247 | CTGGTTTG GGCTAGCTACAACGA TTCACCTG | 16604 |
| 548 | UCAAGCAA A CCACACUG | 7248 | CACTGTCG GGCTAGCTACAACGA TTGCTTCA | 16605 |
| 551 | ACCAAACC A CACUGCCU | 4266 | ACCCAGTC GGCTAGCTACAACGA CCTTTGCT | 16606 |
| 553 | CAAACCAC A CUCCCUUC | 4267 | GAAGCCAC GGCTAGCTACAACGA GTGGTTTC | 16607 |
| 557 | CCACACUC G CUUCUACA | 7249 | TGTAGAAG GGCTAGCTACAACGA CAGTGTGC | 16608 |
| 563 | UGCCUUCU A CACCUCCA | 61 | TGCAGCTC GGCTAGCTACAACGA AGAAGCCA | 16609 |
| 566 | CUUCUACA C CUGCAAAU | 7250 | ATTTGCAG GGCTAGCTACAACGA TCTAGAAC | 16610 |
| 569 | CUACAGCU G CAAAUAUC | 7251 | CATATTTG GGCTAGCTACAACGA AGCTGTAC | 16611 |
| 573 | ACCUCCAA A UAUCUAGC | 7252 | CCTAGATA GGCTAGCTACAACGA TTCCACCT | 16612 |
| 575 | CUCCAAAU A UCUAGCUG | 62 | CACCTACA GGCTAGCTACAACGA ATTTCCAC | 16613 |
| 580 | AAUAUCUA C CUCUACCU | 7253 | ACCTACAC GGCTAGCTACAACGA TACATATT | 16614 |
| 583 | AUCUACCU C UACCUACU | 7254 | ACTACCTA GGCTAGCTACAACGA ACCTACAT | 16615 |
| 585 | CUACCUCU A CCUACUUC | 65 | CAACTACC GGCTAGCTACAACGA ACACCTAC | 16616 |
| 589 | CUCUACCU A CUUCAAAG | 66 | CTTTCAAC GGCTAGCTACAACGA AUCTACAC | 16617 |
| 607 | ACAAGCAA A CACAAUCU | 7255 | AGATTCTC GGCTAGCTACAACGA TTCCTTCT | 16618 |
| 612 | CAAACACA A UCUCCAAU | 7256 | ATTCCACA GGCTAGCTACAACGA TCTCTTTC | 16619 |
| 616 | CACAAUCU C CAAUCUAU | 7257 | ATACATTC GGCTAGCTACAACGA ACATTCTC | 16620 |
| 619 | AAUCUCCA A UCUAUAUA | 7258 | TATATACA GGCTAGCTACAACGA TGCAGATT | 16621 |
| 623 | UCCAAUCU A UAUAUUUA | 71 | TAAATATA GGCTAGCTACAACGA ACATTCCA | 16622 |
| 625 | CAAUCUAU A UAUUUAUU | 72 | AATAAATA GGCTAGCTACAACGA ATACATTC | 16623 |
| 627 | AUCUAUAU A UUUAUUAC | 73 | CTAATAAA GGCTAGCTACAACGA ATATACAT | 16624 |
| 631 | AUAUAUUU A UUACUCAU | 76 | ATCACTAA GGCTAGCTACAACGA AAATATAT | 16625 |
| 635 | AUUUAUUA G UGAUACAG | 7259 | CTGTATCA GGCTAGCTACAACGA TAATAAAT | 16626 |
| 638 | UAUUAGUG A UACAGGUA | 7260 | TACCTGTA GGCTAGCTACAACGA CACTAATA | 16627 |
| 640 | UUAGUGAU A CAGGUAGA | 79 | TCTACCTG GGCTAGCTACAACGA ATCACTAA | 16628 |
| 644 | UGAUACAG G UAGACCUU | 7261 | AAGGTCTA GGCTAGCTACAACGA CTGTATCA | 16629 |
| 648 | ACAGGUAG A CCUUUCGU | 7262 | ACGAAAGG GGCTAGCTACAACGA CTACCTGT | 16630 |
| 655 | GACCUUUC G UAGAGAUG | 7263 | CATCTCTA GGCTAGCTACAACGA GAAAGGTC | 16631 |
| 661 | UCGUAGAG A UGUACAGU | 7264 | ACTGTACA GGCTAGCTACAACGA CTCTACGA | 16632 |
| 663 | GUAGAGAU G UACAGUGA | 7265 | TCACTGTA GGCTAGCTACAACGA ATCTCTAC | 16633 |
| 665 | AGAGAUGU A CAGUGAAA | 85 | TTTCACTG GGCTAGCTACAACGA ACATCTCT | 16634 |
| 668 | GAUGUACA G UGAAAUCC | 7266 | GGATTTCA GGCTAGCTACAACGA TGTACATC | 16635 |
| 673 | ACAGUGAA A UCCCCGAA | 7267 | TTCGGGGA GGCTAGCTACAACGA TTCACTGT | 16636 |
| 682 | UCCCCGAA A UUAUACAC | 7268 | GTGTATAA GGCTAGCTACAACGA TTCGGGGA | 16637 |
| 685 | CCGAAAUU A UACACAUG | 88 | CATGTGTA GGCTAGCTACAACGA AATTTCGG | 16638 |
| 687 | GAAAUUAU A CACAUGAC | 89 | GTCATGTG GGCTAGCTACAACGA ATAATTTC | 16639 |
| 689 | AAUUAUAC A CAUGACUG | 4291 | CAGTCATG GGCTAGCTACAACGA GTATAATT | 16640 |
| 691 | UUAUACAC A UGACUGAA | 4292 | TTCAGTCA GGCTAGCTACAACGA GTGTATAA | 16641 |
| 694 | UACACAUG A CUGAAGGA | 7269 | TCCTTCAG GGCEAGCTACAACGA CATGTGTA | 16642 |
| 708 | GGAAGGGA G CUCGUCAU | 7270 | ATGACGAG GGCTAGCTACAACGA TCCCTTCC | 16643 |
| 712 | GGGAGCUC G UCAUUCCC | 7271 | GGGAATGA GGCTAGCTACAACGA GAGCTCCC | 16644 |
| 715 | AGCUCGUC A UUCCCUGC | 4295 | GCAGGGAA GGCTAGCTACAACGA GACGAGCT | 16645 |
| 722 | CAUUCCCU G CCGGGUUA | 7272 | TAACCCGG GGCTAGCTACAACGA AGGGAATG | 16646 |
| 727 | CCUGCCGG G UUACGUCA | 7273 | TGACGTAA GGCTAGCTACAACGA CCGGCAGG | 16647 |
| 730 | GCCGGGUU A CGUCACCU | 95 | AGGTGACG GGCTAGCTACAACGA AACCCGGC | 16648 |
| 732 | CGGGUUAC G UCACCUAA | 7274 | TTAGGTGA GGCTAGCTACAACGA GTAACCCG | 16649 |
| 735 | GUUACGUC A CCUAACAU | 4300 | ATGTTAGG GGCTAGCTACAACGA GACGTAAC | 16650 |
| 740 | GUCACCUA A CAUCACUG | 7275 | CAGTGATG GGCTAGCTACAACGA TAGGTGAC | 16651 |
| 742 | CACCUAAC A UCACUGUU | 4303 | AACAGTGA GGCTAGCTACAACGA GTTAGGTG | 16652 |
| 745 | CUAACAUC A CUGUUACU | 4304 | AGTAACAG GGCTAGCTACAACGA GATGTTAG | 16653 |
| 748 | ACAUCACU G UUACUUUA | 7276 | TAAAGTAA GGCTAGCTACAACGA AGTGATGT | 16654 |
| 751 | UCACUGUU A CUUUAAAA | 100 | TTTTAAAG GGCTAGCTACAACGA AACAGTGA | 16655 |
| 762 | UUAAAAAA G UUUCCACU | 7277 | AGTGGAAA GGCTAGCTACAACGA TTTTTTAA | 16656 |
| 768 | AAGUUUCC A CUUGACAC | 4308 | GTGTCAAG GGCTAGCTACAACGA GGAAACTT | 16657 |
| 773 | UCCACUUG A CACUUUGA | 7278 | TCAAAGTG GGCTAGCTACAACGA CAAGTGGA | 16658 |
| 775 | CACUUGAC A CUUUGAUC | 4310 | GATCAAAG GGCTAGCTACAACGA GTCAAGTG | 16659 |
| 781 | ACACUUUG A UCCCUGAU | 7279 | ATCAGGGA GGCTAGCTACAACGA CAAAGTGT | 16660 |
| 788 | GAUCCCUG A UGGAAAAC | 7280 | GTTTTCCA GGCTAGCTACAACGA CAGGGATC | 16661 |
| 795 | GAUGGAAA A CGCAUAAU | 7281 | ATTATGCG GGCTAGCTACAACGA TTTCCATC | 16662 |
| 797 | UGGAAAAC G CAUAAUCU | 7282 | AGATTATG GGCTAGCTACAACGA GTTTTCCA | 16663 |
| 799 | GAAAACGC A UAAUCUGG | 4315 | CCAGATTA GGCTAGCTACAACGA GCGTTTTC | 16664 |
| 802 | AACGCAUA A UCUGGGAC | 7283 | GTCCCAGA GGCTAGCTACAACGA TATGCGTT | 16665 |
| 809 | AAUCUGGG A CAGUAGAA | 7284 | TTCTACTG GGCTAGCTACAACGA CCCAGATT | 16666 |
| 812 | CUGGGACA G UAGAAAGG | 7285 | CCTTTCTA GGCTAGCTACAACGA TGTCCCAG | 16667 |
| 821 | UAGAAAGG G CUUGAUGA | 7286 | TGATGAAG GGCTAGCTACAACGA CCTTTCTA | 16668 |
| 826 | AGGGCUUC A UCAUAUCA | 4319 | TGATATGA GGCTAGCTACAACGA GAAGCCCT | 16669 |
| 829 | GCUUGAUG A UAUCAAAU | 4320 | ATTTGATA GGCTAGCTACAACGA GATGAAGC | 16670 |
| 831 | UUGAUGAU A UCAAAUGC | 117 | GCATTTGA GGCTAGCTACAACGA ATGATGAA | 16671 |
| 836 | CAUAUCAA A UGCAACGU | 7287 | ACGTTGCA GGCTAGCTACAACGA TTGATATG | 16672 |
| 838 | UAUCAAAU G CAACGUAC | 7288 | GTACGTTG GGCTAGCTACAACGA ATTTGATA | 16673 |
| 841 | CAAAUGCA A CGUACAAA | 7289 | TTTGTACG GGCTAGCTACAACGA TGCATTTG | 16674 |
| 843 | AAUGCAAC G UACAAAGA | 7290 | TCTTTGTA GGCTAGCTACAACGA GTTGCATT | 16675 |
| 845 | UGCAACGU A CAAAGAAA | 119 | TTTCTTTG GGCTAGCTACAACGA ACGTTGCA | 16676 |
| 853 | ACAAAGAA A UAGGGCUU | 7291 | AAGCCCTA GGCTAGCTACAACGA TTCTTTGT | 16677 |
| 858 | GAAAUAGG G CUUCUGAC | 7292 | GTCAGAAG GGCTAGCTACAACGA CCTATTTC | 16678 |
| 865 | GGCUUCUG A CCUGUGAA | 7293 | TTCACAGG GGCTAGCTACAACGA CAGAAGCC | 16679 |
| 869 | UCUGACCU G UCAAGCAA | 7294 | TTGCTTCA GGCTAGCTACAACGA AGGTCAGA | 16680 |
| 874 | CCUGUGAA G CAACAGUC | 7295 | GACTCTTG GGCTAGCTACAACGA TTCACAGC | 16681 |
| 877 | GUGAACCA A CAGUCAAU | 7296 | ATTGACTG GGCTAGCTACAACGA TGCTTCAC | 16682 |
| 880 | AAGCAACA G UCAAUGGG | 7297 | CCCATTCA GGCTAGCTACAACGA TGTTCCTT | 16683 |
| 884 | AACAGUCA A UCGGCAUU | 7298 | AATCCCCA GGCTAGCTACAACGA TCACTGTT | 16684 |
| 888 | CUCAAUGG G CAUUUCUA | 7299 | TACAAATG GGCTAGCTACAACGA CCATTGAC | 16685 |
| 890 | CAAUGCGC A UCUGUACA | 4331 | TATACAAA GGCTAGCTACAACGA GCCCATTG | 16686 |
| 894 | GGCCAUUU G UAUAAGAC | 7300 | GTCTTATA GGCTAGCTACAACGA AAATGCCC | 16687 |
| 896 | GCAUUUGU A UAAGACAA | 126 | TTGTCTTA GGCTAGCTACAACGA ACAAATCC | 16688 |
| 901 | UGUAUAAC A CAAACUAU | 7301 | ATACTTTG GGCTAGCTACAACGA CTTATACA | 16689 |
| 905 | UAAGACAA A CUAUCUCA | 7302 | TGACATAG GGCTAGCTACAACGA TTGTCTTA | 16690 |
| 908 | GACAAACU A UCUCACAC | 128 | GTGTGAGA GGCTAGCTACAACGA ACTTTGTC | 16691 |
| 913 | ACUACCUC A CACAUCGA | 4335 | TCGATCTC GGCTAGCTACAACGA GAGATAGT | 16692 |
| 915 | UAUCUCAC A CAUCGACA | 4336 | TCTCCATC GGCTAGCTACAACGA GTGAGATA | 16693 |
| 917 | UCUCACAC A UCGACAAA | 4337 | TTTGTCGA GGCTAGCTACAACGA CTGTGAGA | 16694 |
| 921 | ACACAUCC A CAAACCAA | 7303 | TTGGTTTC GGCTAGCTACAACGA CGATCTGT | 16695 |
| 925 | AUCCACAA A CCAAUACA | 7304 | TGTATTGG GGCTAGCTACAACGA TTCTCGAT | 16696 |
| 929 | ACAAACCA A UACAAUCA | 7305 | TGATTCTA GGCTAGCTACAACGA TGGTTTGT | 16697 |
| 931 | AAACCAAU A CAAUCAUA | 132 | TATGATTG GGCTAGCTACAACGA ATTGGTTT | 16698 |
| 934 | CCAAUACA A UCAUACAU | 7306 | ATCTATGA GGCTAGCTACAACGA TCTATTGG | 16699 |
| 937 | AUACAAUC A UAGAUCUC | 4342 | GACATCTA GGCTAGCTACAACGA GATTGTAT | 16700 |
| 941 | AAUCAUAC A UGUCCAAA | 7307 | TTTCCACA GGCTAGCTACAACGA CTATCATT | 16701 |
| 943 | UCAUACAU G UCCAAAUA | 7308 | TATTTCGA GGCTAGCTACAACGA ATCTATGA | 16702 |
| 949 | AUGUCCAA A UAAGCACA | 7309 | TGTCCTTA GGCTAGCTACAACGA TTGCACAT | 16703 |
| 953 | CCAAAUAA G CACACCAC | 7310 | GTCCTGTG GGCTAGCTACAACGA TTATTTGC | 16704 |
| 955 | AAAUAACC A CACCACCC | 4345 | CCCTGCTG GGCTAGCTACAACGA GCTTATTT | 16705 |
| 957 | AUAACCAC A CCACCCCC | 4346 | GGGCGTCC GGCTAGCTACAACGA CTGCTTAT | 16706 |
| 960 | ACCACACC A CGCCCACU | 4348 | ACTGGCCG GGCTAGCTACAACGA GGTGTCCT | 16707 |
| 962 | CACACCAC G CCCAGUCA | 7311 | TGACTGCC GGCTAGCTACAACGA GTGGTCTG | 16708 |
| 967 | CACCCCCA G UCAAAUUA | 7312 | TAATTTGA GGCTAGCTACAACGA TGCGCGTG | 16709 |
| 972 | CCAGUCAA A UUACUCAG | 7313 | CTAAGTAA GGCTAGCTACAACGA TTCACTCC | 16710 |
| 975 | CUCAAAUU A CUUACACC | 139 | CCTCTAAG GGCTAGCTACAACGA AATTTGAC | 16711 |
| 983 | ACUUACAC G CCAUACUC | 7314 | CACTATCC GGCTAGCTACAACGA CTCTAACT | 16712 |
| 986 | UACAGGCC A UACUCUUC | 4355 | CAAGACTA GGCTAGCTACAACGA CCCCTCTA | 16713 |
| 988 | GAGCCCAU A CUCUCCUC | 142 | GACAAGAG GGCTAGCTACAACGA ATCCCCTC | 16714 |
| 994 | AUACUCUU G UCCUCAAU | 7315 | ATTCACCA GGCTAGCTACAACGA AAGAGTAT | 16715 |
| 1001 | UCUCCUCA A UUGUACUG | 7316 | CACTACAA GGCTAGCTACAACGA TCACCACA | 16716 |
| 1004 | CCUCAAUU G UACUCCUA | 7317 | TAGCACTA GGCTAGCTACAACGA AATTCACC | 16717 |
| 1006 | UCAAUUCU A CUCCUACC | 148 | CGTAGCAC GGCTAGCTACAACGA ACAATTCA | 16718 |
| 1009 | AUUCUACU G CUACCACU | 7318 | ACTCCTAC GGCTAGCTACAACGA ACTACAAT | 16719 |
| 1012 | CUACUGCU A CCACUCCC | 149 | CCCACTGG GGCTAGCTACAACGA ACCACTAC | 16720 |
| 1015 | CUCCUACC A CUCCCUUG | 4364 | CAAGGGAG GGCTAGCTACAACGA CCTACCAC | 16721 |
| 1025 | UCCCUUCA A CACCACAG | 7319 | CTCTCCTC GGCTAGCTACAACGA TCAACCCA | 16722 |
| 1027 | CCUUCAAC A CCACACUU | 4369 | AACTCTCG GGCTAGCTACAACGA GTTCAAGG | 16723 |
| 1033 | ACACGACA G UUCAAAUG | 7320 | CATTTGAA GGCTAGCTACAACGA TCTCGTGT | 16724 |
| 1039 | GAGUUCAA A UGACCUGG | 7321 | CCAGGTCA GGCTAGCTACAACGA TTGAACTC | 16725 |
| 1042 | UUCAAAUG A CCUGGAGU | 7322 | ACTCCACG GGCTAGCTACAACGA CATTTGAA | 16726 |
| 1049 | GACCUGGA G UUACCCUG | 7323 | CAGGGTAA GGCTAGCTACAACGA TCCAGGTC | 16727 |
| 1052 | CUGGAGUU A CCCUGAUG | 155 | CATCACGG GGCTAGCTACAACGA AACTCCAC | 16728 |
| 1058 | UUACCCUG A UGAAAAAA | 7324 | TTTTTTCA GGCTAGCTACAACGA CAGGGTAA | 16729 |
| 1067 | UGAAAAAA A UAAGAGAG | 7325 | CTCTCTTA GGCTAGCTACAACGA TTTTTTCA | 16730 |
| 1075 | AUAACAGA G CUUCCGUA | 7326 | TACGGAAC GGCTAGCTACAACGA TCTCTTAT | 16731 |
| 1081 | GAGCUUCC G UAAGCCCA | 7327 | TCGCCTTA GGCTAGCTACAACGA CCAAGCTC | 16732 |
| 1086 | UCCGUAAC G CGACGAAU | 7328 | ATTCGTCG GGCTAGCTACAACGA CTTACCGA | 16733 |
| 1089 | GUAAGGCG A CGAAUUGA | 7329 | TCAATTCG GGCTAGCTACAACGA CGCCTTAC | 16734 |
| 1093 | GGCGACGA A UUGACCAA | 7330 | TTGGTCAA GGCTAGCTACAACGA TCGTCGCC | 16735 |
| 1097 | ACGAAUUG A CCAAAGCA | 7331 | TGCTTTGG GGCTAGCTACAACGA CAATTCGT | 16736 |
| 1103 | UGACCAAA G CAAUUCCC | 7332 | GGGAATTG GGCTAGCTACAACGA TTTGGTCA | 16737 |
| 1106 | CCAAAGCA A UUCCCAUG | 7333 | CATGGCAA GGCTAGCTACAACGA TCCTTTCG | 16738 |
| 1112 | CAAUUCCC A UCCCAACA | 4383 | TCTTCGCA GGCTAGCTACAACGA CCCAATTG | 16739 |
| 1114 | AUUCCCAU G CCAACAUA | 7334 | TATGTTGC GGCTAGCTACAACGA ATGGGAAT | 16740 |
| 1118 | CCAUGCCA A CAUAUUCU | 7335 | ACAATATG GGCTAGCTACAACGA TGCCATCC | 16741 |
| 1141 | UUCUUACU A UUCACAAA | 171 | TTTCTCAA GGCTAGCTACAACGA AGTAAGAA | 16748 |
| 1145 | UACUAUUC A CAAAAUCC | 7338 | GCATTTTG GGCTAGCTACAACGA CAATAGTA | 16749 |
| 1150 | UUCACAAA A UCCACAAC | 7339 | GTTCTCCA GGCTAGCTACAACGA TTTGTCAA | 16750 |
| 1152 | CACAAAAU G CAGAACAA | 7340 | TTCTTCTC GGCTAGCTACAACGA ATTTTGTC | 16751 |
| 1157 | AAUGCACA A CAAACACA | 7341 | TGTCTTTC GGCTAGCTACAACGA TCTCCATT | 16752 |
| 1163 | CAACAAAC A CAAACGAC | 7342 | GTCCTTTG GGCTAGCTACAACGA CTTTCTTC | 16753 |
| 1170 | CACAAACC A CUUUAUAC | 7343 | CTATAAAC GGCTAGCTACAACGA CCTTTCTC | 16754 |
| 1175 | ACCACUUU A UACUUCUC | 175 | CACAACTA GGCTAGCTACAACGA AAACTCCT | 16755 |
| 1177 | GACUUUAU A CUUGUCCU | 176 | ACCACAAC GGCTAGCTACAACGA ATAAACTC | 16756 |
| 1181 | UUAUACUU G UCCUCUAA | 7344 | TTACACCA GGCTAGCTACAACGA AACTATAA | 16757 |
| 1184 | UACUUCUC G UCUAACCA | 7345 | TCCTTACA GGCTAGCTACAACGA CACAACTA | 16758 |
| 1186 | CUUCUCCU G UAACCACU | 7346 | ACTCCTTA GGCTAGCTACAACGA ACCACAAC | 16759 |
| 1193 | UCUAACCA G UCCACCAU | 7347 | ATCCTCCA GGCTAGCTACAACGA TCCTTACA | 16760 |
| 1197 | ACCACUCC A CCAUCAUU | 7348 | AATCATCC GGCTAGCTACAACGA CCACTCCT | 16761 |
| 1200 | ACUCCACC A UCAUUCAA | 4398 | TTCAATCA GGCTAGCTACAACGA CCTCCACT | 16762 |
| 1203 | CCACCAUC A UUCAAAUC | 4399 | CATTTCAA GGCTAGCTACAACGA CATCCTCC | 16763 |
| 1209 | UCAUUCAA A UCUCUUAA | 7349 | TTAACACA GGCTAGCTACAACGA TTCAATCA | 16764 |
| 1213 | UCAAAUCU G UUAACACC | 7350 | CCTCTTAA GGCTAGCTACAACGA ACATTTCA | 16765 |
| 1217 | AUCUCUUA A CACCUCAC | 7351 | CTCACCTC GGCTAGCTACAACGA TAACACAT | 16766 |
| 1219 | CUCUUAAC A CCUCACUC | 4402 | CACTCACC GGCTAGCTACAACGA CTTAACAC | 16767 |
| 1225 | ACACCUCA G UCCAUAUA | 7352 | TATATCCA GGCTAGCTACAACGA TCACCTCT | 16768 |
| 1227 | ACCUCACU G CAUAUAUA | 7353 | TATATATC GGCTAGCTACAACGA ACTCACCT | 16769 |
| 1229 | CUCACUCC A UAUAUAUC | 4406 | CATATATA GGCTAGCTACAACGA CCACTCAC | 16770 |
| 1231 | CACUCCAU A UAUAUCAU | 187 | ATCATATA GGCTAGCTACAACGA ATCCACTC | 16771 |
| 1233 | CUCCAUAU A UAUCAUAA | 188 | TTATCATA GGCTAGCTACAACGA ATATCCAC | 16772 |
| 1235 | GCAUAUAU A UGAUAAAG | 189 | CTTTATCA GGCTAGCTACAACGA ATATATGC | 16773 |
| 1238 | UAUAUAUG A UAAAGCAU | 7354 | ATGCTTTA GGCTAGCTACAACGA CATATATA | 16774 |
| 1243 | AUGAUAAA G CAUUGAUG | 7355 | GATGAATC GGCTAGCTACAACGA TTTATCAT | 16775 |
| 1245 | GAUAAAGC A UUCAUCAC | 4407 | CTGATCAA GGCTAGCTACAACGA GCTTTATC | 16776 |
| 1249 | AAGCAUUC A UCACUGUC | 4408 | CACAGTCA GGCTAGCTACAACGA GAATGCTT | 16777 |
| 1252 | CAUUCAUC A CUGUGAAA | 4409 | TTTCACAC GGCTAGCTACAACGA CATGAATC | 16778 |
| 1255 | UGAUGACU G UGAAACAU | 7356 | ATCTTTCA GGCTAGCTACAACGA AGTGATGA | 16779 |
| 1260 | ACUGUCAA A CAUCGAAA | 7357 | TTTCCATG GGCTAGCTACAACGA TTCACACT | 16780 |
| 1262 | UCUGAAAC A UCCAAAAC | 4411 | CTTTTCCA GGCTAGCTACAACGA CTTTCACA | 16781 |
| 1269 | CAUCGAAA A CACCAGCU | 7358 | ACCTGCTC GGCTAGCTACAACGA TTTCCATG | 16782 |
| 1272 | CCAAAACA G CACGUCCU | 7359 | ACCACCTC GGCTAGCTACAACGA TGTTTTCC | 16783 |
| 1276 | AACACCAG G UCCUUGAA | 7360 | TTCAAGCA GGCTAGCTACAACGA CTCCTGTT | 16784 |
| 1278 | CAGCACCU G CUUCAAAC | 7361 | GTTTCAAC GGCTAGCTACAACGA ACCTCCTC | 16785 |
| 1285 | UGCUUGAA A CCCUACCU | 7362 | AGCTACGC GGCTAGCTACAACGA TTCAACCA | 16786 |
| 1288 | UUGAAACC G UACCUGCC | 7363 | CCCACCTA GGCTAGCTACAACGA GCTTTCAA | 16787 |
| 1291 | AAACCCUA G CUGCCAAG | 7364 | CTTCCCAC GGCTAGCTACAACGA TACCGTTT | 16788 |
| 1295 | CCUAGCUG G CAAGCGGU | 7365 | ACCGCTTC GGCTAGCTACAACGA CAGCTACG | 16789 |
| 1299 | CCUGCCAA G CGCUCUUA | 7366 | TAACACCC GGCTAGCTACAACGA TTGCCACC | 16790 |
| 1302 | GGCAACCG G UCUUACCG | 7367 | CGGTAACA GGCTAGCTACAACGA CCCTTCCC | 16791 |
| 1307 | CCGCUCUU A CCGCCUCU | 199 | ACAGCCCC GGCTAGCTACAACGA AAGACCCC | 16792 |
| 1311 | UCUUACCC G CUCUCUAU | 7368 | ATAGACAG GGCTAGCTACAACGA CGGTAAGA | 16793 |
| 1318 | GGCUCUCU A UGAAACUC | 202 | CACTTTCA GGCTAGCTACAACGA ACAGAGCC | 16794 |
| 1324 | CUAUCAAA G UCAAGGCA | 7369 | TCCCTTCA GGCTAGCTACAACGA TTTCATAC | 16795 |
| 1330 | AAGUGAAG G CAUUUCCC | 7370 | GGCAAATG GGCTAGCTACAACGA CTTCACTT | 16796 |
| 1332 | GUCAAGCC A UUUCCCUC | 4423 | GAGGGAAA GGCTAGCTACAACGA CCCTTCAC | 16797 |
| 1341 | UUUCCCUC G CCGGAAGU | 7371 | ACTTCCGC GGCTAGCTACAACGA GACCGAAA | 16798 |
| 1348 | CGCCGCAA G UUCUAUCC | 7372 | CCATACAA GGCTAGCTACAACGA TTCCCCCC | 16799 |
| 1351 | CCCAACUU G UAUCCUUA | 7373 | TAACCATA GGCTAGCTACAACGA AACTTCCC | 16800 |
| 1353 | CAACUUCU A UCCUUAAA | 208 | TTTAACCA GGCTAGCTACAACGA ACAACTTC | 16801 |
| 1356 | CUUCUAUC G UUAAAACA | 7374 | TCTTTTAA GGCTAGCTACAACGA CATACAAC | 16802 |
| 1364 | CUUAAAAC A UCCCUUAC | 7375 | CTAACCCA GGCTAGCTACAACGA CTTTTAAC | 16803 |
| 1368 | AAACAUCC G UUACCUCC | 7376 | CCACCTAA GGCTAGCTACAACGA CCATCTTT | 16804 |
| 1371 | CAUCCCUU A CCUCCCAC | 212 | CTCCCACC GGCTAGCTACAACGA AACCCATC | 16805 |
| 1375 | CCUUACCU G CCACUCAC | 7377 | CTCACTCC GGCTAGCTACAACGA ACCTAACC | 16806 |
| 1378 | UACCUCCC A CUCACAAA | 7378 | TTTCTCAC GGCTAGCTACAACGA CCCACCTA | 16807 |
| 1386 | ACUCACAA A UCUCCUCC | 7379 | CCACCACA GGCTAGCTACAACGA TTCTCACT | 16808 |
| 1390 | ACAAAUCU G CUCCCUAU | 7380 | ATACCCAC GGCTAGCTACAACGA ACATTTCT | 16809 |
| 1394 | AUCUCCUC G CUAUUUCA | 7381 | TCAAATAC GGCTAGCTACAACGA CACCACAT | 16810 |
| 1397 | UCCUCCCU A CUCCACUC | 215 | CACTCAAA GGCTAGCTACAACGA ACCCACCA | 16811 |
| 1402 | CCUAUUUC A CUCCUCCC | 7382 | CCCACCAC GGCTAGCTACAACGA CAAATACC | 16812 |
| 1406 | UUUCACUC G UCCCUACU | 7383 | ACTACCCA GGCTAGCTACAACGA CACTCAAA | 16813 |
| 1409 | CACUCCUC G CUACUCCU | 7384 | ACCACTAC GGCTAGCTACAACGA CACCACTC | 16814 |
| 1412 | UCCUCCCU A CUCCUUAA | 219 | TTAACCAC GGCTAGCTACAACGA ACCCACCA | 16815 |
| 1416 | CCCUACUC G UUAAUUAU | 7385 | ATAATTAA GGCTAGCTACAACGA CACTACCC | 16816 |
| 1420 | ACUCCUUA A UUAUCAAC | 7386 | CTTCATAA GGCTAGCTACAACGA TAACCACT | 16817 |
| 1423 | CCUUAAUU A UCAACCAC | 224 | CTCCTTCA GGCTAGCTACAACGA AATTAACC | 16818 |
| 1430 | UAUCAACC A CCUAACUC | 7387 | CACTTACC GGCTAGCTACAACGA CCTTCATA | 16819 |
| 1432 | UCAACCAC G UAACUCAA | 7388 | TTCACTTA GGCTAGCTACAACGA CTCCTTCA | 16820 |
| 1435 | ACCACCUA A CUCAACAC | 7389 | CTCTTCAC GGCTAGCTACAACGA TACCTCCT | 16821 |
| 1445 | UGAAGAGG A UGCAGGGA | 7390 | TCCCTGCA GGCTAGCTACAACGA CCTCTTCA | 16822 |
| 1447 | AAGAGGAU G CAGGGAAU | 7391 | ATTCCCTG GGCTAGCTACAACGA ATCCTCTT | 16823 |
| 1454 | UGCAGGGA A UUAUACAA | 7392 | TTGTATAA GGCTAGCTACAACGA TCCCTGCA | 16824 |
| 1457 | AGGGAAUU A UACAAUCU | 228 | AGATTGTA GGCTAGCTACAACGA AATTCCCT | 16825 |
| 1459 | GGAAUUAU A CAAUCUUG | 229 | CAAGATTG GGCTAGCTACAACGA ATAATTCC | 16826 |
| 1462 | AUUAUACA A UCUUGCUG | 7393 | CAGCAAGA GGCTAGCTACAACGA TGTATAAT | 16827 |
| 1467 | ACAAUCUU G CUGAGCAU | 7394 | ATGCTCAG GGCTAGCTACAACGA AAGATTGT | 16828 |
| 1472 | CUUGCUGA G CAUAAAAC | 7395 | GTTTTATG GGCTAGCTACAACGA TCAGCAAG | 16829 |
| 1474 | UGCUGAGC A UAAAACAG | 4443 | CTGTTTTA GGCTAGCTACAACGA GCTCAGCA | 16830 |
| 1479 | AGCAUAAA A CAGUCAAA | 7396 | TTTGACTG GGCTAGCTACAACGA TTTATGCT | 16831 |
| 1482 | AUAAAACA G UCAAAUGU | 7397 | ACATTTGA GGCTAGCTACAACGA TCTTTTAT | 16832 |
| 1487 | ACAGUCAA A UGUGUUUA | 7398 | TAAACACA GGCTAGCTACAACGA TTCACTGT | 16833 |
| 1489 | AGUCAAAU G UGUUUAAA | 7399 | TTTAAACA GGCTAGCTACAACGA ATTTGACT | 16834 |
| 1491 | UCAAAUGU G UUUAAAAA | 7400 | TTTTTAAA GGCTAGCTACAACGA ACATTTGA | 16835 |
| 1499 | GUUUAAAA A CCUCACUG | 7401 | CAGTGACG GGCTAGCTACAACGA TTTTAAAC | 16836 |
| 1504 | AAAACCUC A CUGCCACU | 4448 | AGTCCCAC GGCTAGCTACAACGA GACCTTTT | 16837 |
| 1507 | ACCUCACU G CCACUCUA | 7402 | TACACTGG GGCTAGCTACAACGA ACTCAGGT | 16838 |
| 1510 | UCACUGCC A CUCUAAUU | 4451 | AATTAGAC GGCTAGCTACAACGA GGCAGTCA | 16839 |
| 1516 | CCACUCUA A UUGUCAAU | 7403 | ATTCACAA GGCTAGCTACAACGA TAGAGTCC | 16840 |
| 1519 | CUCUAAUU G UCAAUCUG | 7404 | CACAETGA GGCTAGCTACAACGA AATTAGAC | 16841 |
| 1523 | AAUUGUCA A UGUCAAAC | 7405 | GTTTCACA GGCTAGCTACAACGA TGACAATT | 16842 |
| 1525 | UUGUCAAU G UGAAACCC | 7406 | GGGTTTCA GGCTAGCTACAACGA ATTCACAA | 16843 |
| 1530 | AAUCUGAA A CCCCACAU | 7407 | ATCTGGCC GGCTAGCTACAACGA TTCACATT | 16844 |
| 1537 | AACCCCAG A UUUACCAA | 7408 | TTCCTAAA GGCTAGCTACAACGA CTGGGCTT | 16845 |
| 1541 | CCAGAUUU A CCAAAAGG | 244 | CCTTTTCC GGCTAGCTACAACGA AAATCTGG | 16846 |
| 1549 | ACGAAAAG G CCGUGUCA | 7409 | TCACACGC GGCTAGCTACAACGA CTTTTCGT | 16847 |
| 1552 | AAAAGGCC G UGUGAUGG | 7410 | CGATGACA GGCTAGCTACAACGA GCCCTTTT | 16848 |
| 1554 | AAGCCCCU G UGAUGCUU | 7411 | AACCATGA GGCTAGCTACAACGA ACGGCCTT | 16849 |
| 1557 | GCCGUCUC A UCGUUUCC | 4460 | CCAAACGA GGCTAGCTACAACGA GACACCGC | 16850 |
| 1560 | GUGUGAUG G UUUCCAGA | 7412 | TCTGGAAA GGCTAGCTACAACGA GATCACAC | 16851 |
| 1568 | GUUUCCAG A CCCGGCUC | 7413 | GAGCCGGC GGCTAGCTACAACGA CTGCAAAC | 16852 |
| 1573 | CACACCCG G CUCUCUAC | 7414 | CTAGAGAC GGCTAGCTACAACGA CGCCTCTC | 16853 |
| 1580 | GGCUCUCU A CCCACUCC | 252 | CCAGTCGC GGCTAGCTACAACGA AGAGAGCC | 16854 |
| 1584 | CUCUACCC A CUCGGCAC | 4470 | CTGCCCAC GGCTAGCTACAACGA GCGTAGAG | 16855 |
| 1589 | CCCACUGC G CACCAGAC | 7415 | GTCTCCTG GGCTAGCTACAACGA CCACTCCC | 16856 |
| 1592 | ACUCGGCA G CAGACAAA | 7416 | TTTGTCTG GGCTAGCTACAACGA TGCCCAGT | 16857 |
| 1596 | GCCAGCAG A CAAAUCCU | 7417 | ACGATTTC GGCTAGCTACAACGA CTGCTCCC | 16858 |
| 1600 | CCACACAA A UCCUCACU | 7418 | ACTCAGCA GGCTAGCTACAACGA TTGTCTCC | 16859 |
| 1606 | AAAUCCUG A CUUCUACC | 7419 | CCTACAAG GGCTAGCTACAACGA CAGGATTT | 16860 |
| 1610 | CCUCACUU G UACCGCAU | 7420 | ATGCCCTA GGCTAGCTACAACGA AACTCAGC | 16861 |
| 1612 | UGACUUCU A CCCCAUAU | 255 | ATATCCGC GGCTAGCTACAACGA ACAACTCA | 16862 |
| 1615 | CUUGUACC G CAUAUGGU | 7421 | ACCATATG GGCTAGCTACAACGA GCTACAAG | 16863 |
| 1617 | UCUACCGC A UAUCCUAU | 4479 | ATACCATA GGCTAGCTACAACGA GCGGTACA | 16864 |
| 1619 | UACCGCAU A UGGUAUCC | 256 | CGATACCA GGCTAGCTACAACGA ATCCGCTA | 16865 |
| 1622 | CGCAUAUC G UAUCCCUC | 7422 | CACCCATA GGCTAGCTACAACGA CATATCCC | 16866 |
| 1624 | CAUAUCCU A UCCCUCAA | 257 | TTCACCCA GGCTAGCTACAACGA ACCATATC | 16867 |
| 1632 | AUCCCUCA A CCUACAAU | 7423 | ATTCTAGC GGCTAGCTACAACGA TCACCGAT | 16868 |
| 1636 | CUCAACCU A CAAUCAAC | 260 | CTTGATTC GGCTAGCTACAACGA ACCTTGAC | 16869 |
| 1639 | AACCUACA A UCAACUCC | 7424 | CCACTTCA GGCTAGCTACAACGA TCTACCTT | 16870 |
| 1644 | ACAAUCAA G UGGUUCUG | 7425 | CAGAACCA GGCTAGCTACAACGA TTGATTGT | 16871 |
| 1647 | AUCAAGUG G UUCUGGCA | 7426 | TGCCAGAA GGCTAGCTACAACGA CACTTGAT | 16872 |
| 1653 | UGGUUCUG G CACCCCUG | 7427 | CAGCGGTG GGCTAGCTACAACGA CAGAACCA | 16873 |
| 1655 | GUUCUGGC A CCCCUGUA | 4489 | TACAGGGG GGCTAGCTACAACGA GCCAGAAC | 16874 |
| 1661 | GCACCCCU G UAACCAUA | 7428 | TATGGTTA GGCTAGCTACAACGA AGGGGTGC | 16875 |
| 1664 | CCCCUGUA A CCAUAAUC | 7429 | CATTATGC GGCTAGCTACAACGA TACAGGGG | 16876 |
| 1667 | CUGUAACC A UAAUCAUU | 4495 | AATGATTA GGCTAGCTACAACGA GGTTACAG | 16877 |
| 1670 | UAACCAUA A UCAUUCCG | 7430 | CGGAATGA GGCTAGCTACAACGA TATGGTTA | 16878 |
| 1673 | CCAUAAUC A UUCCGAAG | 4496 | CTTCGGAA GGCTAGCTACAACGA GATTATGG | 16879 |
| 1681 | AUUCCGAA G CAAGGUGU | 7431 | ACACCTTG GGCTAGCTACAACGA TTCGGAAT | 16880 |
| 1686 | GAAGCAAG G UGUGACUU | 7432 | AAGTCACA GGCTAGCTACAACGA CTTGCTTC | 16881 |
| 1688 | AGCAAGGU G UGACUUUU | 7433 | AAAAGTCA GGCTAGCTACAACGA ACCTTGCT | 16882 |
| 1691 | AAGGUGUG A CUUUUGUU | 7434 | AACAAAAG GGCTAGCTACAACGA CACACCTT | 16883 |
| 1697 | UGACUUUU G UUCCAAUA | 7435 | TATTGGAA GGCTAGCTACAACGA AAAAGTCA | 16884 |
| 1703 | UUGUUCCA A UAAUGAAG | 7436 | CTTCATTA GGCTAGCTACAACGA TGGAACAA | 16885 |
| 1706 | UUCCAAUA A UGAAGAGU | 7437 | ACTCTTCA GGCTAGCTACAACGA TATTCGAA | 16886 |
| 1713 | AAUGAAGA G UCCUUUAU | 7438 | ATAAAGGA GGCTAGCTACAACGA TCTTCATT | 16887 |
| 1720 | ACUCCUUU A UCCUGGAU | 278 | ATCCAGGA GGCTAGCTACAACGA AAAGGACT | 16888 |
| 1727 | UAUCCUCG A UGCUGACA | 7439 | TCTCAGCA GGCTAGCTACAACGA CCACGATA | 16889 |
| 1729 | UCCUGCAU G CUGACAGC | 7440 | CCTGTCAC GGCTAGCTACAACGA ATCCAGGA | 16890 |
| 1733 | GCAUGCUG A CAGCAACA | 7441 | TGTTCCTG GGCTAGCTACAACGA CACCATCC | 16891 |
| 1736 | UCCUCACA G CAACAUGG | 7442 | CCATGTTG GGCTAGCTACAACGA TGTCACCA | 16892 |
| 1739 | UGACAGCA A CAUGGCAA | 7443 | TTCCCATC GGCTAGCTACAACGA TGCTGTCA | 16893 |
| 1741 | ACAGCAAC A UCGCAAAC | 4509 | GTTTCCCA GGCTAGCTACAACGA GTTCCTGT | 16894 |
| 1748 | CAUGGGAA A CAGAAUUC | 7444 | CAATTCTG GGCTAGCTACAACGA TTCCCATC | 16895 |
| 1753 | CAAACACA A UUCACAGC | 7445 | GCTCTCAA GGCTAGCTACAACGA TCTCTTTC | 16896 |
| 1760 | AAUUCAGA G CAUCACUC | 7446 | GACTGATG GGCTAGCTACAACGA TCTCAATT | 16897 |
| 1762 | UUCAGAGC A UCACUCAG | 4511 | CTGAGTCA GGCTAGCTACAACGA CCTCTCAA | 16898 |
| 1765 | AGAGCAUC A CUCAGCGC | 4512 | GCGCTGAG GGCTAGCTACAACGA GATCCTCT | 16899 |
| 1770 | AUCACUCA G CGCAUGCC | 7447 | GCCATCCC GGCTAGCTACAACGA TGAGTGAT | 16900 |
| 1772 | CACUCAGC G CAUCCCAA | 7448 | TTGCCATC GGCTAGCTACAACGA GCTCAGTG | 16901 |
| 1774 | CUCACCGC A UGGCAAUA | 4515 | TATTGCCA GGCTAGCTACAACGA CCGCTCAC | 16902 |
| 1777 | AGCGCAUC G CAAUAAUA | 7449 | TATTATTC GGCTAGCTACAACGA CATGCGCT | 16903 |
| 1780 | GCAUGGCA A UAAUAGAA | 7450 | TTCTATTA GGCTAGCTACAACGA TCCCATGC | 16904 |
| 1783 | UCGCAAUA A UACAACGA | 7451 | TCCTTCTA GGCTAGCTACAACGA TATTGCCA | 16905 |
| 1796 | ACCAAAGA A UAACAUCG | 7452 | CCATCTTA GGCTAGCTACAACGA TCTTTCCT | 16906 |
| 1801 | AGAAUAAC A UGCCUAGC | 7453 | CCTACCCA GGCTAGCTACAACGA CTTATTCT | 16907 |
| 1804 | AUAAGAUG G CUAGCACC | 7454 | GGTCCTAG GGCTAGCTACAACGA CATCTTAT | 16908 |
| 1808 | CAUCGCUA G CACCUUGG | 7455 | CCAAGGTG GGCTAGCTACAACGA TACCCATC | 16909 |
| 1810 | UCCCUACC A CCUUCCUU | 4518 | AACCAAGC GGCTAGCTACAACGA CCTACCCA | 16910 |
| 1816 | CCACCUUC G UUCUGCCU | 7456 | AGCCACAA GGCTAGCTACAACGA CAACGTCC | 16911 |
| 1819 | CCUUGCUU G UCCCUGAC | 7457 | CTCAGCCA GGCTAGCTACAACGA AACCAACC | 16912 |
| 1822 | UCCUUCUC G CUCACUCU | 7458 | ACACTCAG GGCTAGCTACAACGA CACAACCA | 16913 |
| 1826 | UGUCCCUC A CUCUACAA | 7459 | TTCTACAC GGCTAGCTACAACGA CACCCACA | 16914 |
| 1834 | ACUCUAGA A UUUCUGGA | 7460 | TCCACAAA GGCTAGCTACAACGA TCTAGACT | 16915 |
| 1843 | UUUCUCCA A UCUACAUU | 7461 | AATCTACA GGCTAGCTACAACGA TCCACAAA | 16916 |
| 1847 | UCCAAUCU A CAUUUCCA | 295 | TCCAAATC GGCTAGCTACAACGA ACATTCCA | 16917 |
| 1849 | CAAUCUAC A UUUCCAUA | 4526 | TATCCAAA GGCTAGCTACAACGA CTACATTC | 16918 |
| 1853 | CUACAUUU G CAUACCUU | 7462 | AACCTATC GGCTAGCTACAACGA AAATGTAC | 16919 |
| 1855 | ACAUUUGC A UAGCUUCC | 4527 | GGAAGCTA GGCTAGCTACAACGA GCAAATGT | 16920 |
| 1858 | UUUGCAUA G CUUCCAAU | 7463 | ATTGGAAG GGCTAGCTACAACGA TATGCAAA | 16921 |
| 1865 | ACCUUCCA A UAAAGUUG | 7464 | CAACTTTA GGCTAGCTACAACGA TGGAAGCT | 16922 |
| 1870 | CCAAUAAA G UUGGGACU | 7465 | AGTCCCAA GGCTAGCTACAACGA TTTATTGG | 16923 |
| 1876 | AAGUUGGC A CUGUGGGA | 7466 | TCCCACAG GGCTAGCTACAACGA CCCAACTT | 16924 |
| 1879 | UUGGCACU G UGGGAAGA | 7467 | TCTTCCCA GGCTAGCTACAACGA AGTCCCAA | 16925 |
| 1889 | GGGAAGAA A CAUAAGCU | 7468 | AGCTTATG GGCTAGCTACAACGA TTCTTCCC | 16926 |
| 1891 | GAAGAAAC A UAAGCUUU | 4532 | AAAGCTTA GGCTAGCTACAACGA GTTTCTTC | 16927 |
| 1895 | AAACAUAA G CUUUUAUA | 7469 | TATAAAAG GGCTAGCTACAACGA TTATGTTT | 16928 |
| 1901 | AAGCUUUU A UAUCACAG | 307 | CTGTGATA GGCTAGCTACAACGA AAAAGCTT | 16929 |
| 1903 | GCUUUUAU A UCACAGAU | 308 | ATCTGTGA GGCTAGCTACAACGA ATAAAAGC | 16930 |
| 1906 | UUUAUAUC A CAGAUGUG | 4534 | CACATCTG GGCTAGCTACAACGA GATATAAA | 16931 |
| 1910 | UAUCACAG A UGUGCCAA | 7470 | TTGGCACA GGCTAGCTACAACGA CTGTGATA | 16932 |
| 1912 | UCACAGAU G UGCCAAAU | 7471 | ATTTGGCA GGCTAGCTACAACGA ATCTGTGA | 16933 |
| 1914 | ACAGAUGU G CCAAAUGG | 7472 | CCATTTGG GGCTAGCTACAACGA ACATCTGT | 16934 |
| 1919 | UGUGCCAA A UGGGUUUC | 7473 | GAAACCCA GGCTAGCTACAACGA TTGGCACA | 16935 |
| 1923 | CCAAAUGG U UUUGAUGU | 7474 | ACATGAAA GGCTAGCTACAACGA CCATTTGG | 16936 |
| 1928 | UGGGUUUC A UGUUAACU | 4538 | AGTTAACA GGCTAGCTACAACGA GAAACCCA | 16937 |
| 1930 | GGUUUCAU G UUAACUUG | 7475 | CAAGTTAA GGCTAGCTACAACGA ATGAAACC | 16938 |
| 1934 | UGAUGUUA A CUUGGAAA | 7476 | TTTCCAAG GGCTAGCTACAACGA TAACATGA | 16939 |
| 1945 | UGGAAAAA A UGCCGACG | 7477 | CGTCGGCA GGCTAGCTACAACGA TTTTTCCA | 16940 |
| 1947 | GAAAAAAU U CCGACGGA | 7478 | TCCGTCGG GGCTAGCTACAACGA ATTTTTTC | 16941 |
| 1951 | AAAUGCCG A CGGAAGGA | 7479 | TCCTTCCG GGCTAGCTACAACGA CGGCATTT | 16942 |
| 1964 | AGGAGAUG A CCUGAAAC | 7480 | GTTTCAGG GGCTAGCTACAACGA CCTCTCCT | 16943 |
| 1971 | GACCUGAA A CUGUCUUG | 7481 | CAAGACAG GGCTAGCTACAACGA TTCAGGTC | 16944 |
| 1974 | CUGAAACU G UCUUGCAC | 7482 | GTGCAAGA GGCTAGCTACAACGA AGTTTCAG | 16945 |
| 1979 | ACUGUCUU U CACAGUUA | 7483 | TAACTGTG GGCTAGCTACAACGA AAGACAGT | 16946 |
| 1981 | UUUCUUGC A CAGUUAAC | 4545 | GTTAACTG GGCTAGCTACAACGA GCAAGACA | 16947 |
| 1984 | CUUGCACA G UUAACAAG | 7484 | CTTGTTAA GGCTAGCTACAACGA TGTGCAAG | 16948 |
| 1988 | CACAGUUA A CAAGUUCU | 7485 | AGAACTTG GGCTAGCTACAACGA TAACTGTG | 16949 |
| 1992 | GUUAACAA U UUCUUAUA | 7486 | TATAAGAA GGCTAGCTACAACGA TTGTTAAC | 16950 |
| 1998 | AAGUUCUU A UACAGAGA | 323 | TCTCTGTA GGCTAGCTACAACGA AAGAACTT | 16951 |
| 2000 | GUUCUUAU A CAGAGACG | 324 | CGTCTCTG GGCTAGCTACAACGA ATAAGAAC | 16952 |
| 2006 | AUACAGAG A CGUUACUU | 7487 | AAGTAACG GGCTAGCTACAACGA CTCTGTAT | 16953 |
| 2008 | ACAGAGAC U UUACUUGG | 7488 | CCAAUTAA GGCTAGCTACAACGA GTCTCTGT | 16954 |
| 2011 | GAGACGUU A CUUGGAUU | 326 | AATCCAAG GGCTAGCTACAACGA AACGTCTC | 16955 |
| 2017 | UUACUUGG A UUUUACUG | 7489 | CAGTAAAA GGCTAGCTACAACGA CCAAGTAA | 16956 |
| 2022 | UGGAUUUU A CUGCGGAC | 331 | GTCCGCAG GGCTAGCTACAACGA AAAATCCA | 16957 |
| 2025 | AUUUUACU G CGGACAGU | 7490 | ACTGTCCG GGCTAGCTACAACGA AUTAAAAT | 16958 |
| 2029 | UACUGCGG A CAGUUAAU | 7491 | ATTAACTG GGCTAGCTACAACGA CCGCAGTA | 16959 |
| 2032 | UGCGGACA G UUAAUAAC | 7492 | GTTATTAA GGCTAGCTACAACGA TGTCCGCA | 16960 |
| 2036 | GACAGUUA A UAACAGAA | 7493 | TTCTGTTA GGCTAGCTACAACGA TAACTGTC | 16961 |
| 2039 | AGUUAAUA A CAGAACAA | 7494 | TTGTTCTG GGCTAGCTACAACGA TATTAACT | 16962 |
| 2044 | AUAACAGA A CAAUGCAC | 7495 | GTGCATTG GGCTAGCTACAACGA TCTGTTAT | 16963 |
| 2047 | ACAGAACA A UGCACUAC | 7496 | GTAGTGCA GUCTAGCTACAACGA TGTTCTGT | 16964 |
| 2049 | AGAACAAU U CACUACAG | 7497 | CTGTAGTG GGCTAGCTACAACGA ATTGTTCT | 16965 |
| 2051 | AACAAUGC A CUACAGUA | 4555 | TACTGTAG GGCTAGCTACAACGA GCATTGTT | 16966 |
| 2054 | AAUGCACU A CAGUAUUA | 335 | TAATACTG GGCTAGCTACAACGA AGTGCATT | 16967 |
| 2057 | UCACUACA U UAUUAUCA | 7498 | TGCTAATA GUCTAGCTACAACGA TGTAUTGC | 16968 |
| 2059 | ACUACAGU A UUAGCAAG | 336 | CTTGCTAA GGCTAGCTACAACGA ACTGTAGT | 16969 |
| 2063 | CAGUAUUA G CAAGCAAA | 7499 | TTTGCTTG GGCTAGCTACAACGA TAATACTG | 16970 |
| 2067 | AUUAGCAA G CAAAAAAU | 7500 | ATTTTTTC GGCTAGCTACAACGA TTGCTAAT | 16971 |
| 2074 | AGCAAAAA A UGGCCAUC | 7501 | GATGGCCA GGCTAGCTACAACGA TTTTTGCT | 16972 |
| 2077 | AAAAAAUG G CCAUCACU | 7502 | AGTGATGG GGCTAGCTACAACGA CATTTTTT | 16973 |
| 2080 | AAAUGGCC A UCACUAAG | 4561 | CTTAGTGA GGCTAGCTACAACGA GGCCATTT | 16974 |
| 2083 | UGGCCAUC A CUAAGGAG | 4562 | CTCCTTAG GGCTAGCTACAACGA GATGGCCA | 16975 |
| 2091 | ACUAAGGA G CACUCCAU | 7503 | ATG3AGTG GGCTAGCTACAACGA TCCTTAGT | 16976 |
| 2093 | UAAGGAGC A CUCCAUCA | 4564 | TGATGGAG GGCTAGCTACAACGA GCTCCTTA | 16977 |
| 2098 | AGCACUCC A UCACUCUU | 4567 | AAGAGTGA GGCTAGCTACAACGA GGAGTCCT | 16978 |
| 2101 | ACUCCAUC A CUCUUAAU | 4568 | ATTAACAG GGCTAGCTACAACGA GATGCACT | 16979 |
| 2108 | CACUCUUA A UCUUACCA | 7504 | TGGTAAGA GGCTAGCTACAACGA TAAGAGTG | 16980 |
| 2113 | UUAAUCUU A CCAUGAUG | 348 | CATGATGC GGCTAGCTACAACGA AAGATTAA | 16981 |
| 2116 | AUCUUACC A UGAUGAAU | 4573 | ATTCATGA GGCTAGCTACAACGA GGTAAGAT | 16982 |
| 2119 | UCACCAUC A UGAAUGUU | 4574 | AACATTCA GGCTAGCTACAACGA GATGGTAA | 16983 |
| 2123 | CAUGAUGA A UGUUUCCC | 7505 | GGCAAACA GGCTAGCTACAACGA TCATGATG | 16984 |
| 2125 | UGAUGAAU G UUUCCCUG | 7506 | CAGGGAAA GGCTAGCTACAACGA ATTCATCA | 16985 |
| 2133 | CUUUCCCU G CAAGAUUC | 7507 | CAATCTTC GGCTAGCTACAACGA AGCGAAAC | 16986 |
| 2138 | CCUCCAAC A UUCACGCA | 7508 | TCCCTCAA GGCTAGCTACAACGA CTTGCAGC | 16987 |
| 2144 | AGAUUCAC G CACCUAUC | 7509 | CATAGGTG GGCTAGCTACAACGA CTCAATCT | 16988 |
| 2146 | AUUCACGC A CCUAUGCC | 4580 | GCCATAGG GGCTAGCTACAACGA GCCTCAAT | 16989 |
| 2150 | AGCCACCU A UGCCUCCA | 355 | TUCACUCA GGCTAGCTACAACGA AGCTGCCT | 16990 |
| 2152 | GCACCUAU G CCUGCACA | 7510 | TCTGCAGG GGCTACCTACAACGA ATACGTGC | 16991 |
| 2156 | CUAUGCCU G CAGAGCCA | 7511 | TGGCTCTG GGCTAGCTACAACGA AGGCATAC | 16992 |
| 2161 | CCUGCAGA G CCAGGAAU | 7512 | ATTCCTGC GGCTAGCTACAACGA TCTCCAGG | 16993 |
| 2168 | ACCCACGA A UCUAUACA | 7513 | TGTATACA GGCTAGCTACAACGA TCCTGCCT | 16994 |
| 2170 | CCAGCAAU G UAUACACA | 7514 | TGTCTATA GGCTAGCTACAACGA ATTCCTGG | 16995 |
| 2172 | ACGAAUGU A UACACAGC | 356 | CCTGTGTA GGCTAGCTACAACGA ACATTCCT | 16996 |
| 2174 | GAAUGUAU A CACACCGG | 357 | CCCCTGTG GGCTAGCTACAACGA ATACATTC | 16997 |
| 2176 | AUGUAUAC A CAGGGGAA | 4588 | TTCCCCTG GGCTAGCTACAACGA GTATACAT | 16998 |
| 2188 | CGGAACAA A UCCUCCAG | 7515 | CTGGACGA GGCTAGCTACAACGA TTCTTCCC | 16999 |
| 2206 | AGAAACAA A UUACAAUC | 7516 | CATTGTAA GGCTAGCTACAACGA TTCTTTCT | 17000 |
| 2209 | AAGAAAUU A CAAUCAGA | 361 | TCTGATTG GGCTAGCTACAACGA AATTTCTT | 17001 |
| 2212 | AAAUUACA A UCACACAU | 7517 | ATCTCTGA GGCTAGCTACAACGA TGTAATTT | 17002 |
| 2219 | AAUCACAC A UCACGAAC | 7518 | CTTCCTGA GGCTAGCTACAACGA CTCTGATT | 17003 |
| 2227 | AUCAGCAA G CACCACAC | 7519 | GTATCGTG GGCTAGCTACAACGA TTCCTGAT | 17004 |
| 2229 | CACGAAGC A CCAUACCU | 4597 | AGGTATCG GGCTAGCTACAACGA CCTTCCTG | 17005 |
| 2232 | CAACCACC A UACCUCCU | 4599 | ACCAGCTA GGCTAGCTACAACGA GGTGCTTC | 17006 |
| 2234 | AGCACCAU A CCUCCUGC | 364 | CCAGGACG GGCTAGCTACAACGA ATCGTCCT | 17007 |
| 2241 | UACCUCCU G CGAAACCU | 7520 | ACGTTTCG GGCTAGCTACAACGA AGCACCTA | 17008 |
| 2246 | CCUGCCAA A CCUCACUG | 7521 | CACTCACC GGCTAGCTACAACGA TTCCCAGC | 17009 |
| 2252 | AAACCUCA G UGAUCACA | 7522 | TCTGATCA GGCTAGCTACAACGA TCACCTTT | 17010 |
| 2255 | CCUCACUG A UCACACAC | 7523 | CTCTGTGA GGCTAGCTACAACGA CACTCACC | 17011 |
| 2258 | CACUCAUC A CACAGUCC | 4607 | CCACTCTC GGCTAGCTACAACGA CATCACTC | 17012 |
| 2260 | CUGAUGAC A CACUCCCC | 4608 | CCCCACTC GGCTAGCTACAACGA CTCATCAC | 17013 |
| 2263 | AUCACACA G UCGCCAUC | 7524 | CATCCCCA GGCTAGCTACAACGA TGTGTCAT | 17014 |
| 2266 | ACACACUC G CCAUCAGC | 7525 | CCTCATCC GGCTAGCTACAACGA CACTGTCT | 17015 |
| 2269 | CACUGGCC A UCACCACU | 4611 | ACTCCTCA GGCTAGCTACAACGA CCCCACTC | 17016 |
| 2273 | CCCCAUCA G CACUUCCA | 7526 | TCCAACTC GGCTAGCTACAACGA TGATCCCC | 17017 |
| 2276 | CAUCAGCA G UUCCACCA | 7527 | TGGTGGAA GGCTAGCTACAACGA TGCTGATG | 17018 |
| 2281 | GCAGUUCC A CCACUUUA | 4615 | TAAAGTGG GGCTAGCTACAACGA GGAACTGC | 17019 |
| 2284 | GUUCCACC A CUUUAGAC | 4617 | GTCTAAAG GGCTAGCTACAACGA GGTGGAAC | 17020 |
| 2291 | CACUUUAG A CUGUGAUG | 7528 | CATGACAG GGCTAGCTACAACGA CTAAAGTG | 17021 |
| 2294 | UUUAGACU G UGAUGCUA | 7529 | TAGCATGA GGCTAGCTACAACGA AGTCTAAA | 17022 |
| 2297 | AGACUGUC A UGCUAAUG | 4620 | CATTAGCA GGCTAGCTACAACGA GACAGTCT | 17023 |
| 2299 | ACUGUCAU G CUAAUGGU | 7530 | ACCATTAG GGCTAGCTACAACGA ATGACAGT | 17024 |
| 2303 | UGAUGCUA A UGGUGUCC | 7531 | GGACACCA GGCTAGCTACAACGA TAGCATGA | 17025 |
| 2306 | UGCUAAUG G UGUCCCCG | 7532 | CGGGGACA GGCTAGCTACAACGA CATTAGCA | 17026 |
| 2308 | CUAAUGGU G UCCCCGAG | 7533 | CTCGGGGA GGCTAGCTACAACGA ACCATTAG | 17027 |
| 2316 | GUCCCCGA G CCUCAGAU | 7534 | ATCTGAGG GGCTAGCTACAACGA TCGGGGAC | 17028 |
| 2323 | AGCCUCAG A UCACUUGG | 7535 | CCAAGTGA GGCTAGCTACAACGA CTGAGGCT | 17029 |
| 2326 | CUCAGAUC A CUUGGUUU | 4628 | AAACCAAG GGCTAGCTACAACGA GATCTGAG | 17030 |
| 2331 | AUCACUUG G UUUAAAAA | 7536 | TTTTTAAA GGCTAGCTACAACGA CAAGTGAT | 17031 |
| 2339 | GUUUAAAA A CAACCACA | 7537 | TGTGGTTG GGCTAGCTACAACGA TTTTAAAC | 17032 |
| 2342 | UAAAAACA A CCACAAAA | 7538 | TTTTGTGG GGCTAGCTACAACGA TGTTTTTA | 17033 |
| 2345 | AAACAACC A CAAAAUAC | 4632 | GTATTTTG GGCTAGCTACAACGA GGTTGTTT | 17034 |
| 2350 | ACCACAAA A UACAACAA | 7539 | TTGTTGTA GGCTAGCTACAACGA TTTGTGGT | 17035 |
| 2352 | CACAAAAU A CAACAAGA | 383 | TCTTGTTG GGCTAGCTACAACGA ATTTTGTG | 17036 |
| 2355 | AAAAUACA A CAAGAGCC | 7540 | GGCTCTTG GGCTAGCTACAACGA TGTATTTT | 17037 |
| 2361 | CAACAAGA G CCUGGAAU | 7541 | ATTCCAGG GGCTAGCTACAACGA TCTTGTTG | 17038 |
| 2368 | AGCCUGGA A UUAUUUUA | 7542 | TAAAATAA GGCTAGCTACAACGA TCCAGGCT | 17039 |
| 2371 | CUGGAAUU A UUUUAGGA | 385 | TCCTAAAA GGCTAGCTACAACGA AATTCCAG | 17040 |
| 2379 | AUUUUAGG A CCAGGAAG | 7543 | CTTCCTGG GGCTAGCTACAACGA CCTAAAAT | 17041 |
| 2387 | ACCAGGAA G CACCACUC | 7544 | GCGTGCTG GGCTAGCTACAACGA TTCCTGGT | 17042 |
| 2390 | AGGAAGCA G CACGCUGU | 7545 | ACAGCGTG GGCTAGCTACAACGA TGCTTCCT | 17043 |
| 2392 | GAAGCAGC A CGCUGUUU | 4641 | AAACAGCG GGCTAGCTACAACGA GCTGCTTC | 17044 |
| 2394 | AGCAGCAC G CUGUUUAU | 7546 | ATAAACAG GGCTAGCTACAACGA GTGCTGCT | 17045 |
| 2397 | AGCACGCU G UUUAUUGA | 7547 | TCAATAAA GGCTAGCTACAACGA AGCGTGCT | 17046 |
| 2401 | CGCUGUUU A UUGAAAGA | 392 | TCTTTCAA GGCTAGCTACAACGA AAACAGCG | 17047 |
| 2410 | UUGAAAGA G UCACAGAA | 7548 | TTCTGTGA GGCTAGCTACAACGA TCTTTCAA | 17048 |
| 2413 | AAAGAGUC A CAGAAGAG | 4643 | CTCTTCTG GGCTAGCTACAACGA GACTCTTT | 17049 |
| 2423 | AGAAGAGG A UGAAGGUG | 7549 | CACCTTCA GGCTAGCTACAACGA CCTCTTCT | 17050 |
| 2429 | GGAUGAAG G UGUCUAUC | 7550 | GATAGACA GGCTAGCTACAACGA CTTCATCC | 17051 |
| 2431 | AUGAAGGU G UCUAUCAC | 7551 | GTGATAGA GGCTAGCTACAACGA ACCTTCAT | 17052 |
| 2435 | AGGUGUCU A UCACUGCA | 396 | TGCAGTGA GGCTAGCTACAACGA AGACACCT | 17053 |
| 2438 | UGUCUAUC A CUGCAAAG | 4646 | CTTTGCAG GGCTAGCTACAACGA GATAGACA | 17054 |
| 2441 | CUAUCACU G CAAAGCCA | 7552 | TGGCTTTG GGCTAGCTACAACGA AGTGATAG | 17055 |
| 2446 | ACUGCAAA G CCACCAAC | 7553 | GTTGGTGG GGCTAGCTACAACGA TTTGCAGT | 17056 |
| 2449 | GCAAAGCC A CCAACCAG | 4650 | CTGGTTGG GGCTAGCTACAACGA GGCTTTGC | 17057 |
| 2453 | AGCCACCA A CCAGAAGG | 7554 | CCTTCTGG GGCTAGCTACAACGA TGGTGGCT | 17058 |
| 2462 | CCAGAAGG G CUCUGUGG | 7555 | CCACAGAG GGCTAGCTACAACGA CCTTCTGG | 17059 |
| 2467 | AGGGCUCU G UGGAAAGU | 7556 | ACTTTCCA GGCTAGCTACAACGA AGAGCCCT | 17060 |
| 2474 | UGUGGAAA G UUCAGCAU | 7557 | ATGCTGAA GGCTAGCTACAACGA TTTCCACA | 17061 |
| 2479 | AAAGUUCA G CAUACCUC | 7558 | GAGGTATG GGCTAGCTACAACGA TGAACTTT | 17062 |
| 2481 | AGUUCAGC A UACCUCAC | 4658 | GTGAGGTA GGCTAGCTACAACGA GCTGAACT | 17063 |
| 2483 | UUCAGCAU A CCUCACUG | 401 | CAGTGAGG GGCTAGCTACAACGA ATGCTGAA | 17064 |
| 2488 | CAUACCUC A CUGUUCAA | 4661 | TTGAACAG GGCTAGCTACAACGA GAGGTATG | 17065 |
| 2491 | ACCUCACU G UUCAAGGA | 7559 | TCCTTGAA GGCTAGCTACAACGA AGTGAGGT | 17066 |
| 2500 | UUCAAGGA A CCUCGGAC | 7560 | GTCCGAGG GGCTAGCTACAACGA TCCTTGAA | 17067 |
| 2507 | AACCUCGG A CAAGUCUA | 7561 | TAGACTTG GGCTAGCTACAACGA CCGACGTT | 17068 |
| 2511 | UCGGACAA G UCUAAUCU | 7562 | AGATTAGA GGCTAGCTACAACGA TTGTCCGA | 17069 |
| 2516 | CAAGUCUA A UCUGGAGC | 7563 | GCTCCAGA GGCTAGCTACAACGA TAGACTTG | 17070 |
| 2523 | AAUCUGGA G CUGAUCAC | 7564 | GTGATCAG GGCTAGCTACAACGA TCCAGATT | 17071 |
| 2527 | UGGAGCUG A UCACUCUA | 7565 | TAGAGTGA GGCTAGCTACAACGA CAGCTCCA | 17072 |
| 2530 | AGCUGAUC A CUCUAACA | 4670 | TGTTAGAG GGCTAGCTACAACGA GATCAGCT | 17073 |
| 2536 | UCACUCUA A CAUGCACC | 7566 | CGTGCATG GGCTAGCTACAACGA TAGAGTGA | 17074 |
| 2538 | ACUCUAAC A UCCACCUG | 4673 | CAGGTGCA GGCTAGCTACAACGA GTTAGAGT | 17075 |
| 2540 | UCUAACAU G CACCUGUG | 7567 | CACAGGTG GGCTAGCTACAACGA ATUTTAGA | 17076 |
| 2542 | UAACAUGC A CCUGUGUG | 4674 | CACACAGG GGCTAGCTACAACGA GCATGTTA | 17077 |
| 2546 | AUGCACCU G UGUGGCUG | 7568 | CAGCCACA GGCTAGCTACAACGA AGGTGCAT | 17078 |
| 2548 | GCACCUGU G UGGCUGCG | 7569 | CGCAGCCA GGCTAGCTACAACGA ACAGGTGC | 17079 |
| 2551 | CCUGUGUG G CUGCGACU | 7570 | AGTCGCAG GGCTAGCTACAACGA CACACAGG | 17080 |
| 2554 | GUGUGGCU G CUACUCUC | 7571 | GACAGTCG GGCTAGCTACAACGA AGCCACAC | 17081 |
| 2557 | UGGCUGCG A CUCUCUUC | 7572 | GAAGAGAG GGCTAGCTACAACGA CGCACCCA | 17082 |
| 2568 | CUCUCCUG G CUCCUAUU | 7573 | AATAGGAG GGCTAGCTACAACGA CAGAAGAG | 17083 |
| 2574 | UCCCUCCU A UUAACCCU | 417 | ACCGTTAA GGCTAGCTACAACGA ACCACCCA | 17084 |
| 2578 | UCCUAUUA A CCCUCCUU | 7574 | AAGCACGC GGCTAGCTACAACGA TAATACGA | 17085 |
| 2587 | CCCUCCUU A UCCGAAAA | 422 | TTTTCCCA GGCTAGCTACAACGA AACGAGCC | 17086 |
| 2596 | UCCGAAAA A UGAAAAGG | 7575 | CCTTTTCA GGCTAGCTACAACGA TTTTCCGA | 17087 |
| 2604 | AUGAAAAG G UCUUCUUC | 7576 | GAACAAGA GGCTAGCTACAACGA CTTTTCAT | 17088 |
| 2617 | CUUCUGAA A UAAAGACU | 7577 | AGTCTTTA GGCTAGCTACAACGA TTCAGAAC | 17089 |
| 2623 | AAAUAAAG A CUGACUAC | 7578 | GTAGTCAG GGCTAGCTACAACGA CTTTATTT | 17090 |
| 2627 | AAAGACUG A CUACCUAU | 7579 | ATAGGTAG GGCTAGCTACAACGA CAGTCTTT | 17091 |
| 2630 | CACUGACU A CCUAUCAA | 430 | TTCATACG GGCTAGCTACAACGA AGTCAGTC | 17092 |
| 2634 | GACUACCU A UCAAUUAU | 431 | ATAATTGA GGCTAGCTACAACGA ACGTAGTC | 17093 |
| 2638 | ACCUAUCA A UUAUAAUG | 7580 | CATTATAA GGCTAGCTACAACGA TCATAGGT | 17094 |
| 2641 | UAUCAAUU A UAAUGCAC | 434 | CTCCATTA GGCTAGCTACAACGA AATTCATA | 17095 |
| 2644 | CAAUUAUA A UGCACCCA | 7581 | TCCCTCCA GGCTAGCTACAACGA TATAATTG | 17096 |
| 2648 | UAUAAUGG A CCCACAUG | 7582 | CATCTCGG GGCTAGCTACAACGA CCATTATA | 17097 |
| 2654 | CGACCCAG A UGAAGUUC | 7583 | CAACTTCA GGCTAGCTACAACGA CTGGCTCC | 17098 |
| 2659 | CACAUGAA G UUCCUUUG | 7584 | CAAAGGAA GGCTAGCTACAACGA TTCATCTC | 17099 |
| 2669 | UCCUUUGG A UGACCAGU | 7585 | ACTCCTCA GGCTAGCTACAACGA CCAAAGGA | 17100 |
| 2673 | UUCGAUCA G CACUCUCA | 7586 | TCACACTC GGCTAGCTACAACGA TCATCCAA | 17101 |
| 2676 | GAUGACCA G UCUCACCC | 7587 | CCCTCACA GGCTAGCTACAACGA TGCTCATC | 17102 |
| 2678 | UCAGCAGU G UGACCGCC | 7588 | CCCCCTCA GGCTAGCTACAACGA ACTCCTCA | 17103 |
| 2682 | CACUCUCA G CCCCUCCC | 7589 | CCCACCCG GGCTAGCTACAACGA TCACACTC | 17104 |
| 2685 | UCUGACCG G CUCCCUUA | 7590 | TAAGCCAG GGCTAGCTACAACGA CCCTCACA | 17105 |
| 2693 | CCUCCCUU A UGAUGCCA | 442 | TCCCATCA GGCTAGCTACAACGA AACCCACC | 17106 |
| 2696 | CCCUUAUC A UCCCAGCA | 7591 | TCCTCCCA GGCTAGCTACAACGA CATAACCC | 17107 |
| 2698 | CUUAUCAU G CCACCAAG | 7592 | CTTCCTCC GGCTAGCTACAACGA ATCATAAG | 17108 |
| 2702 | UGAUGCCA G CAACUCCC | 7593 | CCCACTTC GGCTAGCTACAACGA TGCCATCA | 17109 |
| 2706 | CCCACCAA G UGGCACUU | 7594 | AACTCCCA GGCTAGCTACAACGA TTCCTCCC | 17110 |
| 2712 | AAGUCCCA G UUUCCCCC | 7595 | CGCCCAAA GGCTAGCTACAACGA TCCCACTT | 17111 |
| 2716 | CCCAGUUU G CCCCCCAC | 7596 | CTCCCGCC GGCTAGCTACAACGA AAACTCCC | 17112 |
| 2727 | CCGGACAC A CUUAAACU | 7597 | ACTTTAAG GGCTAGCTACAACGA CTCTCCCC | 17113 |
| 2733 | ACACUUAA A CUCCCCAA | 7598 | TTGCCCAG GGCTAGCTACAACGA TTAACTCT | 17114 |
| 2738 | UAAACUCC G CAAAUCAC | 7599 | CTCATTTC GGCTAGCTACAACGA CCACTTTA | 17115 |
| 2742 | CUGGGCAA A UCACUUGG | 7600 | CCAAGTGA GGCTAGCTACAACGA TTGCCCAG | 17116 |
| 2745 | GGCAAAUC A CUUGGAAG | 4717 | CTTCCAAG GGCTAGCTACAACGA GATTTGCC | 17117 |
| 2758 | GAAGAGGG G CUUUUGGA | 7601 | TCCAAAAG GGCTAGCTACAACGA CCCTCTTC | 17118 |
| 2770 | UUGGAAAA G UGGUUCAA | 7602 | TTGAACCA GGCTAGCTACAACGA TTTTCCAA | 17119 |
| 2773 | GAAAAGUG G UUCAAGCA | 7603 | TGCTTGAA GGCTAGCTACAACGA CACTTTTC | 17120 |
| 2779 | UGCUUCAA G CAUCAGCA | 7604 | TGCTCATC GGCTAGCTACAACGA TTGAACCA | 17121 |
| 2781 | CUUCAACC A UCACCAUU | 4721 | AATGCTCA GGCTAGCTACAACGA CCTTCAAC | 17122 |
| 2785 | AACCAUCA G CAUUUGCC | 7605 | GCCAAATC GGCTAGCTACAACGA TGATGCTT | 17123 |
| 2787 | GCAUCACC A UUUGGCAU | 4723 | ATCCCAAA GGCTAGCTACAACGA GCTGATGC | 17124 |
| 2792 | AGCAUUUG G CAUUAAGA | 7606 | TCTTAATC GGCTAGCTACAACGA CAAATGCT | 17125 |
| 2794 | CAUUUCCC A UUAAGAAA | 4724 | TTTCTTAA GGCTAGCTACAACGA GCCAAATC | 17126 |
| 2802 | AUUAAGAA A UCACCUAC | 7607 | GTACGTCA GGCTAGCTACAACGA TTCTTAAT | 17127 |
| 2805 | AACAAAUC A CCUACCUG | 4725 | CACGTACC GGCTAGCTACAACGA GATTTCTT | 17128 |
| 2809 | AAUCACCU A CCUCCCGG | 460 | CCGGCACG GGCTAGCTACAACGA ACGTGATT | 17129 |
| 2811 | UCACCUAC G UGCCGCAC | 7608 | GTCCGGCA GGCTAGCTACAACGA GTAGGTCA | 17130 |
| 2813 | ACCUACCU G CCCGACUC | 7609 | CAGTCCCC GGCTAGCTACAACGA ACGTACCT | 17131 |
| 2818 | CGUCCCGG A CUGUGCCU | 7610 | ACCCACAC GGCTAGCTACAACGA CCGGCACG | 17132 |
| 2821 | GCCCGACU G UGCCUCUC | 7611 | CACACCCA GGCTAGCTACAACGA AGTCCCCC | 17133 |
| 2824 | CCACUCUG G CUGUGAAA | 7612 | TTTCACAC GGCTAGCTACAACGA CACACTCC | 17134 |
| 2827 | CUCUCCCU G UGAAAAUG | 7613 | CATTTTCA GGCTAGCTACAACGA ACCCACAG | 17135 |
| 2833 | CUGUGAAA A UGCUGAAA | 7614 | TTTCACCA GGCTAGCTACAACGA TTTCACAG | 17136 |
| 2835 | CUGAAAAU G CUCAAACA | 7615 | TCTTTCAC GGCTAGCTACAACGA ATTTTCAC | 17137 |
| 2848 | AACACCCC G CCACCCCC | 7616 | GCCCGTGG GGCTAGCTACAACGA CCCCTCTT | 17138 |
| 2851 | AGGGCGCC A CCCCCACC | 4733 | CCTCGCCC GGCTAGCTACAACGA CGCCCCCT | 17139 |
| 2854 | GGGCCACC G CCAGCCAG | 7617 | CTCGCTGC GGCTAGCTACAACGA CCTCGCCC | 17140 |
| 2858 | CACGGCCA G CGAGUACA | 7618 | TGTACTCG GGCTAGCTACAACGA TCGCCGTC | 17141 |
| 2862 | GCCAGCGA G UACAAAGC | 7619 | CCTTTCTA GGCTAGCTACAACGA TCCCTGCC | 17142 |
| 2864 | CACCCAGU A CAAAGCUC | 461 | CACCTTTC GGCTAGCTACAACGA ACTCGCTG | 17143 |
| 2869 | AGUACAAA G CUCUGAUG | 7620 | CATCAGAG GGCTAGCTACAACGA TTTCTACT | 17144 |
| 2875 | AAGCUCUG A UGACUCAG | 7621 | CTCAGTCA GGCTAGCTACAACGA CACACCTT | 17145 |
| 2878 | CUCUGAUG A CUCACCUA | 7622 | TACCTCAC GGCTAGCTACAACGA CATCACAC | 17146 |
| 2883 | AUCACUCA G CUAAAAAU | 7623 | ATTTTTAC GGCTAGCTACAACGA TCACTCAT | 17147 |
| 2890 | ACCUAAAA A UCUUCACC | 7624 | CCTCAACA GGCTAGCTACAACGA TTTTAGCT | 17148 |
| 2896 | AAAUCUUC A CCCACAUU | 7625 | AATGTCGG GGCTAGCTACAACGA CAACATTT | 17149 |
| 2900 | CUUCACCC A CAUUCGCC | 4744 | CCCCAATC GGCTAGCTACAACGA GCCTCAAC | 17150 |
| 2902 | UCACCCAC A UUCCCCAC | 4745 | CTGCCCAA GGCTAGCTACAACGA GTGGGTCA | 17151 |
| 2906 | CCACAUUC G CCACCAUC | 7626 | CATCCTCC GGCTAGCTACAACGA CAATCTCC | 17152 |
| 2909 | CAUUGCCC A CCAUCUGA | 4747 | TCACATCC GGCTAGCTACAACGA CCCCAATC | 17153 |
| 2912 | UCCCCACC A UCUCAACC | 4749 | CCTTCACA GGCTAGCTACAACGA GCTCCCCA | 17154 |
| 2918 | CCAUCUGA A CCUCCUUA | 7627 | TAACCACC GGCTAGCTACAACGA TCAGATGG | 17155 |
| 2920 | AUCUCAAC G UCCUUAAC | 7628 | CTTAACCA GGCTAGCTACAACGA CTTCAGAT | 17156 |
| 2923 | UCAACCUC G UUAACCUG | 7629 | CAGCTTAA GGCTAGCTACAACGA CACCTTCA | 17157 |
| 2927 | CGUCGUUA A CCUGCUCC | 7630 | CCACCACC GGCTAGCTACAACGA TAACCACC | 17158 |
| 2931 | CUUAACCU G CUCCCACC | 7631 | CCTCCCAC GGCTAGCTACAACGA ACCTTAAC | 17159 |
| 2938 | UGCUGGGA G CCUGCACC | 7632 | GGTCCACC GGCTAGCTACAACGA TCCCACCA | 17160 |
| 2942 | CCCACCCU G CACCAACC | 7633 | CCTTCCTC GGCTAGCTACAACGA ACCCTCCC | 17161 |
| 2944 | GAGCCUGC A CCAAGCAA | 4756 | TTGCTTGC GGCTAGCTACAACGA CCACCCTC | 17162 |
| 2949 | UCCACCAA G CAACGAGC | 7634 | CCTCCTTC GGCTAGCTACAACGA TTGGTGCA | 17163 |
| 2958 | CAACCACC G CCUCUCAU | 7635 | ATCACACC GGCTAGCTACAACGA CCTCCTTC | 17164 |
| 2965 | GGCCUCUG A UGGUGAUU | 7636 | AATCACCA GGCTAGCTACAACGA CACAGGCC | 17165 |
| 2968 | CUCUGAUG G UGAUUGUU | 7637 | AACAATCA GGCTAGCTACAACGA CATCAGAG | 17166 |
| 2971 | UGAUGGUG A UUGUUGAA | 7638 | TTCAACAA GGCTAGCTACAACGA CACCATCA | 17167 |
| 2974 | UGGUGAUU G UUGAAUAC | 7639 | GTATTCAA GGCTAGCTACAACGA AATCACCA | 17168 |
| 2979 | AUUGUUGA A UACUGCAA | 7640 | TTGCAGTA GGCTAGCTACAACGA TCAACAAT | 17169 |
| 2981 | UGUUGAAU A CUGCAAAU | 473 | ATTTGCAG GGCTAGCTACAACGA ATTCAACA | 17170 |
| 2984 | UGAAUACU G CAAAUAUG | 7641 | CATATTTG GGCTAGCTACAACGA AGTATTCA | 17171 |
| 2988 | UACUCCAA A UAUGGAAA | 7642 | TTTCCATA GGCTAGCTACAACGA TTGCAGTA | 17172 |
| 2990 | CUGCAAAU A UGGAAAUC | 474 | GATTTCCA GGCTAGCTACAACGA ATTTGCAG | 17173 |
| 2996 | AUAUGGAA A UCUCUCCA | 7643 | TGGAGAGA GGCTAGCTACAACGA TTCCATAT | 17174 |
| 3005 | UCUCUCCA A CUACCUCA | 7644 | TGAGGTAG GGCTAGCTACAACGA TGGAGAGA | 17175 |
| 3008 | CUCCAACU A CCUCAAGA | 478 | TCTTGAGG GGCTAGCTACAACGA AGTTGGAG | 17176 |
| 3017 | CCUCAAGA G CAAACGUG | 7645 | CACGTTTC GGCTAGCTACAACGA TCTTGAGG | 17177 |
| 3021 | AAGAGCAA A CGUGACUU | 7646 | AAGTCACG GGCTAGCTACAACGA TTGCTCTT | 17178 |
| 3023 | GAGCAAAC G UGACUUAU | 7647 | ATAAGTCA GGCTAGCTACAACGA GTTTGCTC | 17179 |
| 3026 | CAAACGUG A CUUAUUUU | 7648 | AAAATAAC GGCTAGCTACAACGA CACCTTTC | 17180 |
| 3030 | CGUGACUU A UUUUUUCU | 481 | ACAAAAAA GGCTAGCTACAACGA AAGTCACC | 17181 |
| 3041 | UUUUCUCA A CAAGCAUG | 7649 | CATCCTTG GGCTAGCTACAACGA TGAGAAAA | 17182 |
| 3047 | CAACAAGG A UGCAGCAC | 7650 | CTGCTGCA GGCTAGCTACAACGA CCTTGTTG | 17183 |
| 3049 | ACAACGAU G CACCACUA | 7651 | TACTCCTC GGCTAGCTACAACGA ATCCTTCT | 17184 |
| 3052 | AGGAUGCA G CACUACAC | 7652 | CTGTACTC GGCTAGCTACAACGA TCCATCCT | 17185 |
| 3054 | GAUGCAGC A CUACACAU | 4779 | ATGTCTAC GGCTAGCTACAACGA GCTGCATC | 17186 |
| 3057 | GCACCACU A CACAUGGA | 489 | TCCATGTG GGCTAGCTACAACGA ACTCCTGC | 17187 |
| 3059 | AGCACUAC A CAUGGAUC | 4781 | GCTCCATG GGCTAGCTACAACGA CTAGTCCT | 17188 |
| 3061 | CACUACAC A UGGAGCCU | 4782 | AGCCTCCA GGCTAGCTACAACGA CTGTACTC | 17189 |
| 3066 | CACAUGGA G CCUAAGAA | 7653 | TTCTTAGG GGCTAGCTACAACGA TCCATGTG | 17190 |
| 3082 | AAGAAAAA A UCCAGCCA | 7654 | TGGCTCCA GGCTAGCTACAACGA TTTTTCTT | 17191 |
| 3087 | AAAAUGGA G CCAGGCCU | 7655 | AGGCCTGC GGCTAGCTACAACGA TCCATTTT | 17192 |
| 3092 | CCACCCAG G CCUGGAAC | 7656 | GTTCCACG GGCTAGCTACAACGA CTCGCTCC | 17193 |
| 3099 | GGCCUGGA A CAACGCAA | 7657 | TTCCCTTG GGCTAGCTACAACGA TCCAGGCC | 17194 |
| 3104 | GGAACAAG G CAAGAAAC | 7658 | GTTTCTTG GGCTAGCTACAACGA CTTGTTCC | 17195 |
| 3111 | GGCAACAA A CCAAGACU | 7659 | ACTCTTGG GGCTAGCTACAACGA TTCTTGCC | 17196 |
| 3117 | AAACCAAG A CUAGAUAG | 7660 | CTATCTAC GGCTAGCTACAACGA CTTGGTTT | 17197 |
| 3122 | AAGACUAC A UAGCGUCA | 7661 | TGACGCTA GGCTAGCTACAACGA CTAGTCTT | 17198 |
| 3125 | ACUACAUA G CGUCACCA | 7662 | TGGTGACG GGCTAGCTACAACGA TATCTAGT | 17199 |
| 3127 | UACAUACC G UCACCACC | 7663 | GCTGGTGA GGCTAGCTACAACGA GCTATCTA | 17200 |
| 3130 | AUACCGUC A CCACCAGC | 4794 | GCTGCTGG GGCTAGCTACAACGA CACGCTAT | 17201 |
| 3134 | CGUCACCA G CAGCCAAA | 7664 | TTTCCCTG GGCTAGCTACAACGA TCGTGACG | 17202 |
| 3137 | CACCACCA G CCAAACCU | 7665 | ACCTTTCC GGCTAGCTACAACGA TCCTCGTC | 17203 |
| 3143 | CACCCAAA G CUUUCCCA | 7666 | TCCCAAAC GGCTAGCTACAACGA TTTCGCTG | 17204 |
| 3148 | AAACCUUU G CCACCUCC | 7667 | CCACCTCC GGCTAGCTACAACGA AAAGCTTT | 17205 |
| 3152 | CUUUCCGA G CUCCCCCU | 7668 | ACCCCCAC GGCTAGCTACAACGA TCCCAAAC | 17206 |
| 3158 | CACCUCCC G CUUUCACG | 7669 | CCTCAAAG GGCTAGCTACAACGA CCCACCTC | 17207 |
| 3170 | UCACCAAC A UAAAACUC | 7670 | CACTTTTA GGCTAGCTACAACGA CTTCCTCA | 17208 |
| 3176 | ACAUAAAA G UCUCACUC | 7671 | CACTCACA GGCTAGCTACAACGA TTTTATCT | 17209 |
| 3182 | AACUCUCA G UGAUGCUC | 7672 | CAACATCA GGCTAGCTACAACGA TCACACTT | 17210 |
| 3185 | UCUCACUG A UCUUCACC | 7673 | CCTCAACA GGCTAGCTACAACGA CACTCACA | 17211 |
| 3187 | UCACUCAU G UUCACCAA | 7674 | TTCCTCAA GGCTAGCTACAACGA ATCACTCA | 17212 |
| 3203 | ACACCAGC A UUCUCACC | 7675 | CCTCACAA GGCTAGCTACAACGA CCTCCTCT | 17213 |
| 3209 | GGAUUCUG A CGGUUUCU | 7676 | AGAAACCG GGCTAGCTACAACGA CAGAATCC | 17214 |
| 3212 | UUCUGACG G UUUCUACA | 7677 | TGTAGAAA GGCTAGCTACAACGA CGTCAGAA | 17215 |
| 3218 | CGGUUUCU A CAACGAGC | 508 | GCTCCTTG GGCTAGCTACAACGA AGAAACCG | 17216 |
| 3225 | UACAAGGA G CCCAUCAC | 7678 | GTGATGGG GGCTAGCTACAACGA TCCTTCTA | 17217 |
| 3229 | AGGAGCCC A UCACUACG | 4809 | CATAGTGA GGCTAGCTACAACGA GGCCTCCT | 17218 |
| 3232 | AGCCCAUC A CUAUGGAA | 4810 | TTCCATAG GGCTAGCTACAACGA GATGGGCT | 17219 |
| 3235 | CCAUCACU A UGGAAGAU | 510 | ATCTTCCA GGCTAGCTACAACGA AGTGATGG | 17220 |
| 3242 | UAUGCAAG A UCUGAUUU | 7679 | AAATCACA GGCTAGCTACAACGA CTTCCATA | 17221 |
| 3247 | AAGAUCUC A UUUCUUAC | 7680 | GTAAGAAA GGCTAGCTACAACGA CAGATCTT | 17222 |
| 3254 | GAUUUCUU A CAGUUUUC | 516 | GAAAACTG GGCTAGCTACAACGA AAGAAATC | 17223 |
| 3257 | UUCUUACA G UUUUCAAC | 7681 | CTTGAAAA GGCTAGCTACAACGA TGTAAGAA | 17224 |
| 3265 | GUUUUCAA G UGGCCAGA | 7682 | TCTCGCCA GGCTAGCTACAACGA TTGAAAAC | 17225 |
| 3268 | UUCAACUC G CCAGAGGC | 7683 | GCCTCTGG GGCTAGCTACAACGA CACTTGAA | 17226 |
| 3275 | GGCCAGAG G CAUGGACU | 7684 | ACTCCATG GGCTAGCTACAACGA CTCTCGCC | 17227 |
| 3277 | CCAGAGGC A UGCACUUC | 4818 | CAACTCCA GGCTAGCTACAACGA CCCTCTGG | 17228 |
| 3282 | GGCAUCCA G UUCCUCUC | 7685 | GACAGCAA GGCTAGCTACAACGA TCCATCCC | 17229 |
| 3288 | GAGUUCCU G UCUUCCAG | 7686 | CTGCAACA GGCTAGCTACAACGA AGGAACTC | 17230 |
| 3300 | UCCAGAAA G UGCAUUCA | 7687 | TGAATCCA GGCTAGCTACAACGA TTTCTCGA | 17231 |
| 3302 | CAGAAACU G CAUUGAUG | 7688 | GATGAATC GGCTAGCTACAACGA ACTTTCTC | 17232 |
| 3304 | GAAACUGC A UUGAUGGG | 4824 | CCGATCAA GGCTAGCTACAACGA CCACTTTC | 17233 |
| 3308 | GUGCAUUC A UCGGGACC | 4825 | GCTCCCCA GGCTAGCTACAACGA CAATGCAC | 17234 |
| 3314 | UGAUGGGC A CCUCCCAG | 7689 | CTCCCAGG GGCTAGCTACAACGA CCCCATGA | 17235 |
| 3319 | GGGACCUC G CACCGACA | 7690 | TCTCGCTG GGCTAGCTACAACGA CAGCTCCC | 17236 |
| 3322 | ACCUGGCA G CGAGAAAC | 7691 | CTTTCTCG GGCTAGCTACAACGA TCCCACCT | 17237 |
| 3329 | AGCGACAA A CAUUCUUU | 7692 | AAAGAATG GGCTAGCTACAACGA TTCTCGCT | 17238 |
| 3331 | CGACAAAC A UUCUUUUA | 4829 | TAAAAGAA GGCTAGCTACAACGA GTTTCTCG | 17239 |
| 3339 | AUUCUUUU A UCUGAGAA | 534 | TTCTCAGA GGCTAGCTACAACGA AAAAGAAT | 17240 |
| 3347 | AUCUGACA A CAACGUGG | 7693 | CCACCTTG GGCTAGCTACAACGA TCTCACAT | 17241 |
| 3350 | UCACAACA A CGUGGUGA | 7694 | TCACCACG GGCTAGCTACAACGA TGTTCTCA | 17242 |
| 3352 | ACAACAAC G UCGUCAAC | 7695 | CTTCACCA GGCTAGCTACAACGA GTTCTTCT | 17243 |
| 3355 | ACAACGUG G UCAACAUU | 7696 | AATCTTCA GGCTAGCTACAACGA CACCTTGT | 17244 |
| 3361 | UGGUGAAG A UUUGUGAU | 7697 | ATCACAAA GGCTAGCTACAACGA CTTCACCA | 17245 |
| 3365 | GAACAUUU G UCAUUUUC | 7698 | CAAAATCA GGCTAGCTACAACGA AAATCTTC | 17246 |
| 3368 | CAUUUCUC A UUUUCCCC | 7699 | CCCCAAAA GGCTAGCTACAACGA CACAAATC | 17247 |
| 3374 | UGAUUUUC G CCUUCCCC | 7700 | CCCCAACC GGCTAGCTACAACGA CAAAATCA | 17248 |
| 3379 | UUCCCCUU G CCCGCCAU | 7701 | ATCCCCCC GGCTAGCTACAACGA AACCCCAA | 17249 |
| 3386 | UCCCCGCC A UAUUUAUA | 7702 | TATAAATA GGCTAGCTACAACGA CCCCGCCA | 17250 |
| 3388 | CCCCCCAU A UUUAUAAC | 542 | CTTATAAA GGCTAGCTACAACGA ATCCCCCC | 17251 |
| 3392 | CCAUAUUU A UAAGAACC | 545 | CCTTCTTA GGCTAGCTACAACGA AAATATCC | 17252 |
| 3398 | UUAUAACA A CCCCCAUU | 7703 | AATCCCGC GGCTAGCTACAACGA TCTTATAA | 17253 |
| 3404 | CAACCCCC A UUAUCUGA | 7704 | TCACATAA GGCTAGCTACAACGA CGGGGTTC | 17254 |
| 3407 | CCCCGAUU A UCUGACAA | 548 | TTCTCACA GGCTAGCTACAACGA AATCGGGG | 17255 |
| 3409 | CCCAUUAU G UCACAAAA | 7705 | TTTTCTCA GGCTAGCTACAACGA ATAATCCG | 17256 |
| 3422 | AAAAGCAC A UACUCCAC | 7706 | CTCCACTA GGCTAGCTACAACGA CTCCTTTT | 17257 |
| 3424 | AACCACAU A CUCCACUU | 549 | AAGTCCAC GGCTAGCTACAACGA ATCTCCTT | 17258 |
| 3429 | CAUACUCC A CUUCCUCU | 7707 | ACACCAAC GGCTAGCTACAACGA CCACTATC | 17259 |
| 3441 | CCUCUCAA A UCCAUCGC | 7708 | CCCATCCA GGCTAGCTACAACGA TTCACACC | 17260 |
| 3445 | UCAAAUCC A UGCCUCCC | 7709 | CCCAGCCA GGCTAGCTACAACGA CCATTTCA | 17261 |
| 3448 | AAUCGAUC G CUCCCCAA | 7710 | TTCCCCAC GGCTAGCTACAACGA CATCCATT | 17262 |
| 3456 | GCUCCCGA A UCUAUCUU | 7711 | AAGATAGA GGCTAGCTACAACGA TCGGGAGC | 17263 |
| 3460 | CCGAAUCU A UCUUUGAC | 556 | GTCAAAGA GGCTAGCTACAACGA AGATTCGG | 17264 |
| 3467 | UAUCUUUG A CAAAAUCU | 7712 | AGATTTTG GGCTAGCTACAACGA CAAAGATA | 17265 |
| 3472 | UUGACAAA A UCUACAGC | 7713 | GCTGTAGA GGCTAGCTACAACGA TTTGTCAA | 17266 |
| 3476 | CAAAAUCU A CAGCACCA | 561 | TGGTGCTG GGCTAGCTACAACGA AGATTTTG | 17267 |
| 3479 | AAUCUACA G CACCAAGA | 7714 | TCTTGGTG GGCTAGCTACAACGA TGTAGATT | 17268 |
| 3481 | UCUACAGC A CCAAGAGC | 4853 | GCTCTTGG GGCTAGCTACAACGA GCTGTAGA | 17269 |
| 3488 | CACCAAGA G CGACGUGU | 7715 | ACACGTCG GGCTAGCTACAACGA TCTTGGTG | 17270 |
| 3491 | CAAGAGCG A CCUGUGGU | 7716 | ACCACACG GGCTAGCTACAACGA CGCTCTTG | 17271 |
| 3493 | AGAGCGAC G UGUGGUCU | 7717 | AGACCACA GGCTAGCTACAACGA GTCGCTCT | 17272 |
| 3495 | AGCGACGU G UGGUCUUA | 7718 | TAAGACCA GGCTAGCTACAACGA ACGTCGCT | 17273 |
| 3498 | GACGUGUG G UCUUACGG | 7719 | CCGTAAGA GGCTAGCTACAACGA CACACGTC | 17274 |
| 3503 | GUGGUCUU A CGGAGUAU | 564 | ATACTCCG GGCTAGCTACAACGA AAGACCAC | 17275 |
| 3508 | CUUACGGA G UAUUGCUG | 7720 | CAGCAATA GGCTAGCTACAACGA TCCGTAAG | 17276 |
| 3510 | UACGGAGU A UUGCUGUG | 565 | CACAGCAA GGCTAGCTACAACGA ACTCCGTA | 17277 |
| 3513 | GGAGUAUU G CUGUGGGA | 7721 | TCCCACAG GGCTAGCTACAACGA AATACTCC | 17278 |
| 3516 | GUAUUGCU G UGGGAAAU | 7722 | ATTTCCCA GGCTAGCTACAACGA AGCAATAC | 17279 |
| 3523 | UGUGGGAA A UCUUCUCC | 7723 | GGAGAAGA GGCTAGCTACAACGA TTCCCACA | 17280 |
| 3536 | CUCCUUAG G UGGGUCUC | 7724 | GAGACCCA GGCTAGCTACAACGA CTAAGGAG | 17281 |
| 3540 | UUAGGUGG G UCUCCACA | 7725 | TATCGAGA GGCTAGCTACAACGA CCACCTAA | 17282 |
| 3546 | GGGUCUCC A UACCCAGG | 4864 | CCTGGGTA GGCTAGCTACAACGA GGAGACCC | 17283 |
| 3548 | CUCUCCAU A CCCAGGAG | 575 | CTCCTGGC GGCTAGCTACAACGA ATGGAGAC | 17284 |
| 3556 | ACCCAGGA G UACAAAUG | 7726 | CATTTGTA GGCTAGCTACAACGA TCCTGGGT | 17285 |
| 3558 | CCAGGAGU A CAAAUGCA | 576 | TCCATTTG GGCTAGCTACAACGA ACTCCTGG | 17286 |
| 3562 | GAGUACAA A UGGAUGAG | 7727 | CTCATCCA GGCTAGCTACAACGA TTGTACTC | 17287 |
| 3566 | ACAAAUGG A UGAGGACU | 7728 | AGTCCTCA GGCTAGCTACAACGA CCATTTGT | 17288 |
| 3572 | GGAUGAGG A CUUUUGCA | 7729 | TGCAAAAG GGCTAGCTACAACGA CCTCATCC | 17289 |
| 3578 | GGACUUUU G CAGUCGCC | 7730 | GGCGACTG GGCTAGCTACAACGA AAAAGTCC | 17290 |
| 3581 | CUUUUGCA G UCGCCUGA | 7731 | TCAGGCGA GGCTAGCTACAACGA TGCAAAAG | 17291 |
| 3584 | UUGCAGUC G CCUGAGGG | 7732 | CCCTCAGG GGCTAGCTACAACGA GACTGCAA | 17292 |
| 3596 | GAGGGAAG G CAUGAGGA | 7733 | TCCTCATG GGCTAGCTACAACGA CTTCCCTC | 17293 |
| 3598 | GGGAAGGC A UGAGGAUG | 4873 | CATCCTCA GGCTAGCTACAACGA GCCTTCCC | 17294 |
| 3604 | GCAUGAGG A UGAGAGCU | 7734 | AGCTCTCA GGCTAGCTACAACGA CCTCATGC | 17295 |
| 3610 | GGAUGAGA G CUCCUGAG | 7735 | CTCAGGAG GGCTAGCTACAACGA TCTCATCC | 17296 |
| 3618 | GCUCCUGA G UACUCUAC | 7736 | GTAGAGTA GGCTAGCTACAACGA TCAGGAGC | 17297 |
| 3620 | UCCUGAGU A CUCUACUC | 582 | GAGTAGAG GGCTAGCTACAACGA ACTCAGGA | 17298 |
| 3625 | AGUACUCU A CUCCUGAA | 584 | TTCAGGAG GGCTAGCTACAACGA AGACTACT | 17299 |
| 3634 | CUCCUGAA A UCUAUCAG | 7737 | CTGATAGA GGCTAGCTACAACGA TTCAGGAG | 17300 |
| 3638 | UGAAAUCU A UCAGAUCA | 587 | TGATCTGA GGCTAGCTACAACGA AGATTTCA | 17301 |
| 3643 | UCUAUCAG A UGAUGCUG | 7738 | CAGCATGA GGCTAGCTACAACGA CTGATAGA | 17302 |
| 3646 | AUCAGAUC A UGCUGGAC | 4884 | GTCCAGCA GGCTAGCTACAACGA GATCTGAT | 17303 |
| 3648 | CAGAUCAU G CUGGACUG | 7739 | CAGTCCAG GGCTAGCTACAACGA ATGATCTG | 17304 |
| 3653 | CAUGCUGG A CUGCUGGC | 7740 | GCCAGCAG GGCTAGCTACAACGA CCAGCATG | 17305 |
| 3656 | GCUGGACU G CUGGCACA | 7741 | TGTGCCAG GGCTAGCTACAACGA AGTCCAGC | 17306 |
| 3660 | GACUGCUG G CACAGAGA | 7742 | TCTCTGTG GGCTAGCTACAACGA CAGCAGTC | 17307 |
| 3662 | CUGCUGGC A CAGAGACC | 4888 | GGTCTCTG GGCTAGCTACAACGA GCCAGCAG | 17308 |
| 3668 | GCACAGAG A CCCAAAAG | 7743 | CTTTTGGG GGCTAGCTACAACGA CTCTGTGC | 17309 |
| 3681 | AAAGAAAG G CCAAGAUU | 7744 | AATCTTGG GGCTAGCTACAACGA CTTTCTTT | 17310 |
| 3687 | AGGCCAAG A UUUGCAGA | 7745 | TCTGCAAA GGCTAGCTACAACGA CTTGGCCT | 17311 |
| 3691 | CAAGAUUU G CAGAACUU | 7746 | AAGTTCTG GGCTAGCTACAACGA AAATCTTG | 17312 |
| 3696 | UUUCCAGA A CUUGUGGA | 7747 | TCCACAAG GGCTAGCTACAACGA TCTCCAAA | 17313 |
| 3700 | CAGAACUU G UGGAAAAA | 7748 | TTTTTCCA GGCTAGCTACAACGA AAGTTCTG | 17314 |
| 3708 | GUGGAAAA A CUAGGUGA | 7749 | TCACCTAG GGCTAGCTACAACGA TTTTCCAC | 17315 |
| 3713 | AAAACUAG G UGAUUUGC | 7750 | GCAAATCA GGCTAGCTACAACGA CTAGTTTT | 17316 |
| 3716 | ACUAGGUG A UUUGCUUC | 7751 | GAAGCAAA GGCTAGCTACAACGA CACCTAGT | 17317 |
| 3720 | GGUGAUUU G CUUCAAGC | 7752 | GCTTGAAG GGCTAGCTACAACGA AAATCACC | 17318 |
| 3727 | UGCUUCAA G CAAAUGUA | 7753 | TACATTTG GGCTAGCTACAACGA TTGAAGCA | 17319 |
| 3731 | UCAAGCAA A UGUACAAC | 7754 | GTTGTACA GGCTAGCTACAACGA TTGCTTGA | 17320 |
| 3733 | AAGCAAAU G UACAACAG | 7755 | CTGTTGTA GGCTAGCTACAACGA ATTTGCTT | 17321 |
| 3735 | GCAAAUGU A CAACAGGA | 598 | TCCTGTTG GGCTAGCTACAACGA ACATTTGC | 17322 |
| 3738 | AAUGUACA A CAGGAUGG | 7756 | CCATCCTG GGCTAGCTACAACGA TGTACATT | 17323 |
| 3743 | ACAACAGG A UGGUAAAG | 7757 | CTTTACCA GGCTAGCTACAACGA CCTGTTCT | 17324 |
| 3746 | ACAGGAUG G UAAAGACU | 7758 | AGTCTTTA GGCTAGCTACAACGA CATCCTGT | 17325 |
| 3752 | UGGUAAAG A CUACAUCC | 7759 | GGATGTAG GGCTAGCTACAACGA CTTTACCA | 17326 |
| 3755 | UAAAGACU A CAUCCCAA | 600 | TTGGGATG GGCTAGCTACAACGA AGTCTTTA | 17327 |
| 3757 | AAGACUAC A UCCCAAUC | 4904 | GATTGGGA GGCTAGCTACAACGA GTAGTCTT | 17328 |
| 3763 | ACAUCCCA A UCAAUGCC | 7760 | GGCATTGA GGCTAGCTACAACGA TGGGATGT | 17329 |
| 3767 | CCCAAUCA A UGCCAUAC | 7761 | GTATGGCA GGCTAGCTACAACGA TGATTGGG | 17330 |
| 3769 | CAAUCAAU G CCAUACUG | 7762 | CAGTATGG GGCTAGCTACAACGA ATTGATTG | 17331 |
| 3772 | UCAAUGCC A UACUGACA | 4910 | TGTCAGTA GGCTAGCTACAACGA GGCATTGA | 17332 |
| 3774 | AAUGCCAU A CUGACAGG | 603 | CCTGTCAG GGCTAGCTACAACGA ATGGCATT | 17333 |
| 3778 | CCAUACUG A CAGGAAAU | 7763 | ATTTCCTG GGCTAGCTACAACGA CAGTATGG | 17334 |
| 3785 | GACAGGAA A UAGUGGGU | 7764 | ACCCACTA GGCTAGCTACAACGA TTCCTGTC | 17335 |
| 3788 | AGGAAAUA G UGGGUUUA | 7765 | TAAACCCA GGCTAGCTACAACGA TATTTCCT | 17336 |
| 3792 | AAUAGUGG G UUUACAUA | 7766 | TATGTAAA GGCTAGCTACAACGA CCACTATT | 17337 |
| 3796 | GUGUGGUG A CAUACUCA | 607 | TGAGTATG GGCTAGCTACAACGA AAACCCAC | 17338 |
| 3798 | GGGUUUAC A UACUCAAC | 4913 | GTTGAGTA GGCTAGCTACAACGA GTAPACCC | 17339 |
| 3800 | GUUUACAU A CUCAACUC | 608 | GAGTTGAG GGCTAGCTACAACGA ATGTAAAC | 17340 |
| 3805 | CAGACUCA A CUCCUGCC | 7767 | GGCAGGAG GGCTAGCTACAACGA TGAGTATG | 17341 |
| 3811 | CAACUCCU G CCUUCUCU | 7768 | AGAGAAGG GGCTAGCTACAACGA AGGAGTTG | 17342 |
| 3824 | CUCUGAGG A CUUCUUCA | 7769 | TGAAGAAG GGCTAGCTACAACGA CCTCAGAG | 17343 |
| 3839 | CAAGGAAA G UAUUUCAG | 7770 | CTGAAATA GGCTAGCTACAACGA TTTCCTTG | 17344 |
| 3841 | AGGAAAGU A UUUCAGCU | 618 | AGCTGAAA GGCTAGCTACAACGA ACTTTCCT | 17345 |
| 3847 | GUAUUUCA G CUCCGAAG | 7771 | CTTCGGAG GGCTAGCTACAACGA TGAAATAC | 17346 |
| 3855 | GCUCCGAA G UUUAAUUC | 7772 | GAATTAAA GGCTAGCTACAACGA TTCGGAGC | 17347 |
| 3860 | GAAGUUUA A UUCAGGAA | 7773 | TTCCTGAA GGCTAGCTACAACGA TAAACTTC | 17348 |
| 3869 | UUCAGGAA G CUCUGAUG | 7774 | CATCAGAG GGCTAGCTACAACGA TTCCTGAA | 17349 |
| 3875 | AAGCUCUG A UGAUGUCA | 7775 | TGACATCA GGCTAGCTACAACGA CAGAGCTT | 17350 |
| 3878 | CUCUGAUG A UGUCAGAU | 7776 | ATCTGACA GGCTAGCTACAACGA CATCAGAG | 17351 |
| 3880 | CUGAUGAU G UCAGAUAU | 7777 | ATATCTGA GGCTAGCTACAACGA ATCATCAG | 17352 |
| 3885 | GAUGUCAG A UAUGUAAA | 7778 | TTTACATA GGCTAGCTACAACGA CTGACATC | 17353 |
| 3887 | UGUCAGAU A UGUAAAUG | 630 | CATTTACA GGCTAGCTACAACGA ATCTGACA | 17354 |
| 3889 | UCAGAUAU G UAAAUGCU | 7779 | AGCATTTA GGCTAGCTACAACGA ATATCTGA | 17355 |
| 3893 | AUAUGUAA A UGCUUUCA | 7780 | TGAAAGCA GGCTAGCTACAACGA TTACATAT | 17356 |
| 3895 | AUGUAAAU G CUUUCAAG | 7781 | CTTGAAAG GGCTAGCTACAACGA ATTTACAT | 17357 |
| 3903 | GCUUUCAA G UUGAUGAG | 7782 | CTCATGAA GGCTAGCTACAACGA TTGAAAGC | 17358 |
| 3907 | UCAAGUUC A UGAGCCUG | 4935 | CAGGCTCA GGCTAGCTACAACGA GAACTTGA | 17359 |
| 3911 | GUUGAUGA G CCUGGAAA | 7783 | TTTCCAGG GGCTAGCTACAACGA TCATGAAC | 17360 |
| 3922 | UGGAAAGA A UCAAAACC | 7784 | GGTTTTGA GGCTAGCTACAACGA TCTTTCCA | 17361 |
| 3928 | GAAUCAAA A CCUUUGAA | 7785 | TTCAAAGG GGCTAGCTACAACGA TTTGATTC | 17362 |
| 3939 | UUUGAAGA A CUUUUACC | 7786 | GGTAAAAG GGCTAGCTACAACGA TCTTCAAA | 17363 |
| 3945 | GAACUUUU A CCGAAUGC | 643 | GCATTCGG GGCTAGCTACAACGA AAAAGTTC | 17364 |
| 3950 | UUUACCGA A UGCCACCU | 7787 | AGGTGGCA GGCTAGCTACAACGA TCGGTAAA | 17365 |
| 3952 | UACCGAAU G CCACCUCC | 7788 | GGAGGTGG GGCTAGCTACAACGA ATTCCGTA | 17366 |
| 3955 | CGAAUGCC A CCUCCAUG | 4944 | CATGGAGG GGCTAGCTACAACGA GGCATTCG | 17367 |
| 3961 | CCACCUCC A UGUUUGAU | 4948 | ATCAAACA GGCTAGCTACAACGA GGAGGTGG | 17368 |
| 3963 | ACCUCCAU G UUUGAUCA | 7789 | TCATCAAA GGCTAGCTACAACGA ATGGAGGT | 17369 |
| 3968 | CAUGUUUG A UGACUACC | 7790 | GGTAGTCA GGCTAGCTACAACGA CAAACATC | 17370 |
| 3971 | GUUUGAUG A CUACCAGG | 7791 | CCTGGTAG GGCTAGCTACAACGA CATCAAAC | 17371 |
| 3974 | UGAUGACU A CCAGGGCG | 647 | CGCCCTCG GGCTAGCTACAACGA AGTCATCA | 17372 |
| 3980 | CUACCAGG G CGACAGCA | 7792 | TCCTCTCG GGCTAGCTACAACGA CCTGGTAG | 17373 |
| 3983 | CCAGGGCG A CAGCAGCA | 7793 | TGCTCCTG GGCTAGCTACAACGA CGCCCTGC | 17374 |
| 3986 | GGGCGACA G CAGCACUC | 7794 | GAGTGCTG GGCTAGCTACAACGA TGTCGCCC | 17375 |
| 3989 | CCACAGCA G CACUCUGU | 7795 | ACACAGTG GGCTAGCTACAACGA TGCTGTCG | 17376 |
| 3991 | ACACCAUC A CUCUGUUG | 4954 | CAACACAG GGCTAGCTACAACGA GCTCCTGT | 17377 |
| 3996 | AGCACUCU G UUGGCCUC | 7796 | GAGGCCAA GGCTAGCTACAACGA AGACTCCT | 17378 |
| 4000 | CUCUCCUC G CCUCUCCC | 7797 | CCGAGAGG GGCTAGCTACAACGA CAACAGAC | 17379 |
| 4009 | CCUCUCCC A UCCUCAAG | 4962 | CTTCACCA GGCTAGCTACAACGA CCGAGACC | 17380 |
| 4011 | UCUCCCAU G CUGAAGCG | 7798 | CGCTTCAG GGCTAGCTACAACGA ATGCCAGA | 17381 |
| 4017 | AUCCUGAA G CGCUUCAC | 7799 | CTCAAGCG GGCTAGCTACAACGA TTCAGCAT | 17382 |
| 4019 | GCUGAACC G CUUCACCU | 7800 | ACGTGAAC GGCTAGCTACAACGA GCTTCAGC | 17383 |
| 4024 | ACCGCCUC A CCUGGACU | 4965 | ACTCCACC GGCTAGCTACAACGA CAACCCCT | 17384 |
| 4030 | UCACCUGC A CUCACAGC | 7801 | CCTGTCAC GGCTAGCTACAACGA CCACGTCA | 17385 |
| 4034 | CUGGACUC A CAGCAAAC | 7802 | GTTTCCTG GGCTAGCTACAACGA CACTCCAG | 17386 |
| 4037 | GACUGACA G CAAACCCA | 7803 | TGCGTTTC GGCTAGCTACAACGA TGTCACTC | 17387 |
| 4041 | GACAGCAA A CCCAAGCC | 7804 | CCCTTGCC GGCTAGCTACAACGA TTGCTGTC | 17388 |
| 4048 | AACCCAAG G CCUCCCUC | 7805 | CACCGAGC GGCTAGCTACAACGA CTTCCGTT | 17389 |
| 4053 | AACCCCUC G CUCAAGAU | 7806 | ATCTTGAC GGCTAGCTACAACGA GAGGCCTT | 17390 |
| 4060 | CCCUCAAG A UUGACUUC | 7807 | CAAGTCAA GGCTAGCTACAACGA CTTGAGCG | 17391 |
| 4064 | CAAGAUUG A CUUGAGAC | 7808 | CTCTCAAC GGCTAGCTACAACGA CAATCTTG | 17392 |
| 4072 | ACUUGAGA G UAACCACU | 7809 | ACTGGTTA GGCTAGCTACAACGA TCTCAACT | 17393 |
| 4075 | UCAGACUA A CCACUAAA | 7810 | TTTACTCC GGCTAGCTACAACGA TACTCTCA | 17394 |
| 4079 | ACUAACCA G UAAAAGUA | 7811 | TACTTTTA GGCTAGCTACAACGA TGCTTACT | 17395 |
| 4085 | CACUAAAA G UAAGCAGU | 7812 | ACTCCTTA GGCTAGCTACAACGA TTTTACTG | 17396 |
| 4092 | ACUAACCA G UCCCCCCU | 7813 | ACCCCCCA GGCTAGCTACAACGA TCCTTACT | 17397 |
| 4098 | CACUCCCC G CUCUCUCA | 7814 | TCACACAC GGCTAGCTACAACGA CCCCACTC | 17398 |
| 4101 | UCCCCCCU G UCUGAUGU | 7815 | ACATCACA GGCTAGCTACAACGA ACCCCCCA | 17399 |
| 4106 | CCUCUCUC A UCUCACCA | 7816 | TCCTCACA GGCTAGCTACAACGA CACACACC | 17400 |
| 4108 | UCUCUCAU G UCACCACC | 7817 | CCTCCTCA GGCTAGCTACAACGA ATCACACA | 17401 |
| 4112 | UGAUGUCA G CACCCCCA | 7818 | TCCCCCTC GGCTAGCTACAACGA TCACATCA | 17402 |
| 4116 | CUCACCAC G CCCACUUU | 7819 | AAACTCCC GGCTAGCTACAACGA CTCCTCAC | 17403 |
| 4121 | CACCCCCA G UUUCUCCC | 7820 | CCCACAAA GGCTAGCTACAACGA TCCCCCTC | 17404 |
| 4127 | CACUUUCU G CCAUUCCA | 7821 | TCCAATCC GGCTAGCTACAACGA ACAAACTC | 17405 |
| 4130 | UUUCUCCC A UUCCACCU | 4990 | ACCTCCAA GGCTAGCTACAACGA CCCACAAA | 17406 |
| 4136 | CCAUUCCA G CUCUCCCC | 7822 | CCCCACAC GGCTAGCTACAACGA TCCAATCC | 17407 |
| 4139 | UUCCACCU G UCCCCACC | 7823 | CCTCCCCA GGCTAGCTACAACGA ACCTCCAA | 17408 |
| 4143 | ACCUCUCC G CACCUCAC | 7824 | CTCACCTC GGCTAGCTACAACGA CCACACCT | 17409 |
| 4145 | CUGUGGGC A CGUCAGCG | 4994 | CGCTGACC GGCTAGCTACAACGA GCCCACAG | 17410 |
| 4147 | GUGGGCAC G UCAGCGAA | 7825 | TTCGCTGA GGCTAGCTACAACGA GTGCCCAC | 17411 |
| 4151 | GCACGUCA G CGAAGGCA | 7826 | TGCCTTCG GGCTAGCTACAACGA TGACGTGC | 17412 |
| 4157 | CAGCGAAG G CAAGCGCA | 7827 | TGCGCTTG GGCTAGCTACAACGA CTTCGCTG | 17413 |
| 4161 | GAAGGCAA G CGCAGGUU | 7828 | AACCTGCG GGCTAGCTACAACGA TTGCCTTC | 17414 |
| 4163 | AGGCAAGC G CAGGUUCA | 7829 | TGAACCTG GGCTAGCTACAACGA GCTTGCCT | 17415 |
| 4167 | AAGCGCAC G UUCACCUA | 7830 | TAGGTGAA GGCTAGCTACAACGA CTGCCCTT | 17416 |
| 4171 | GCAGGUUC A CCUACGAC | 4998 | GTCGTAGG GGCTACCTACAACGA GAACCTGC | 17417 |
| 4175 | GUUCACCU A CGACCACG | 672 | CGTGGTCG GGCTAGCTACAACGA AGGTGAAC | 17418 |
| 4178 | CACCUACG A CCACGCUG | 7831 | CACCGTGG GGCTAGCTACAACGA CGTAGGTG | 17419 |
| 4181 | CUACGACC A CGCUGACC | 5002 | GCTCAGCG GGCTAGCTACAACGA GGTCGTAG | 17420 |
| 4183 | ACGACCAC 0 CUGAGCUC | 7832 | CAGCTCAG GGCTAGCTACAACGA GTGGTCGT | 17421 |
| 4188 | CACGCUGA G CUGGAAAG | 7833 | CTTTCCAG GGCTAGCTACAACGA TCAGCGTG | 17422 |
| 4201 | AAACGAAA A UCCCGUGC | 7834 | GCACGCGA GGCTAGCTACAACGA TTTCCTTT | 17423 |
| 4204 | GGAAAAUC G CGUGCUGC | 7835 | GCAGCACG GGCTAGCTACAACGA GATTTTCC | 17424 |
| 4206 | AAAAUCGC G UGCUGCUC | 7836 | GACCACCA GGCTAGCTACAACGA GCGATTTT | 17425 |
| 4208 | AAUCCCGU G CUGCUCCC | 7837 | GGCACCAC GGCTAGCTACAACGA ACGCGATT | 17426 |
| 4211 | CGCCUCCU G CUCCCCGC | 7838 | GCCGCGAG GGCTAGCTACAACGA ACCACGCG | 17427 |
| 4218 | UCCUCCCC G CCCCCAGA | 7839 | TCTCGGCG GGCTAGCTACAACGA GGGCAGCA | 17428 |
| 4226 | GCCCCCAG A CUACAACU | 7840 | ACTTGTAC GGCTAGCTACAACGA CTGCCCGC | 17429 |
| 4229 | CCCAGACU A CAACUCGG | 675 | CCCAGTTG GGCTAGCTACAACGA ACTCTCGG | 17430 |
| 4232 | AGACUACA A CUCGCUGG | 7841 | CCACCGAG GGCTAGCTACAACGA TCTAGTCT | 17431 |
| 4237 | ACAACUCC G UGGUCCUG | 7842 | CAGGACCA GGCTAGCTACAACGA CCAGTTGT | 17432 |
| 4240 | ACUCGGUG G UCCUGUAC | 7843 | GTACACGA GGCTAGCTACAACGA CACCGAGT | 17433 |
| 4245 | GUGGUCCU G UACUCCAC | 7844 | GTGGAGTA GGCTAGCTACAACGA AGGACCAC | 17434 |
| 4247 | CGUCCUGU A CUCCACCC | 678 | CGGTGGAG GGCTAGCTACAACGA ACAGGACC | 17435 |
| 4252 | UGUACUCC A CCCCACCC | 5022 | CGGTCGGG GGCTAGCTACAACGA GGACTACA | 17436 |
| 4257 | UCCACCCC A CCCAUCUA | 5026 | TAGATGGG GGCTAGCTACAACGA GGGGTGGA | 17437 |
| 4261 | CCCCACCC A UCUAGAGU | 5029 | ACTCTAGA GGCTAGCTACAACGA GGGTGCGG | 17438 |
| 4268 | CAUCUAGA G UUUGACAC | 7845 | CTGTCAAA GGCTAGCTACAACGA TCTAGATG | 17439 |
| 4273 | AGAGUUUG A CACGAACC | 7846 | CCTTCGTG GGCTAGCTACAACGA CAAACTCT | 17440 |
| 4275 | AGUUUGAC A CGAAGCCU | 5031 | AGGCTTCC GGCTAGCTACAACGA GTCAAACT | 17441 |
| 4280 | GACACGAA G CCUUAUUU | 7847 | AAATAAGG GGCTAGCTACAACGA TTCGTCTC | 17442 |
| 4285 | GAACCCUU A UUUCUACA | 685 | TCTAGAAA GGCTAGCTACAACGA AAGGCTTC | 17443 |
| 4295 | UUCUAGAA G CACAUGUG | 7848 | CACATGTG GGCTAGCTACAACGA TTCTAGAA | 17444 |
| 4297 | CUACAAGC A CAUGUCUA | 5035 | TACACATG GGCTAGCTACAACGA CCTTCTAG | 17445 |
| 4299 | AGAAGCAC A UGUCUAUU | 5036 | AATACACA GGCTAGCTACAACGA GTGCTTCT | 17446 |
| 4301 | AAGCACAU G UCUAUUUA | 7849 | TAAATACA GGCTAGCTACAACGA ATGTCCTT | 17447 |
| 4303 | GCACAUGU G UAUUUAUA | 7850 | TATAAATA GGCTAGCTACAACGA ACATGTCC | 17448 |
| 4305 | ACAUGUCU A UUUAUACC | 690 | CCTATAAA GGCTAGCTACAACGA ACACATGT | 17449 |
| 4309 | GUGUAUUU A UACCCCCA | 693 | TGCGGGTA GGCTAGCTACAACGA AAATACAC | 17450 |
| 4311 | GUAUUUAU A CCCCCAGG | 694 | CCTGCGGC GGCTAGCTACAACGA ATAAATAC | 17451 |
| 4322 | CCCAGCAA A CUAGCUUU | 7851 | AAAGCTAG GGCTAGCTACAACGA TTCCTCGC | 17452 |
| 4326 | CCAAACUA G CUUUUGCC | 7852 | CGCAAAAG GGCTAGCTACAACGA TACTTTCC | 17453 |
| 4332 | UAGCUUUU G CCAGUAUU | 7853 | AATACTGC GGCTAGCTACAACGA AAAACCTA | 17454 |
| 4336 | UUUUGCCA G UAUUAUGC | 7854 | GCATAATA GGCTAGCTACAACGA TGGCAAAA | 17455 |
| 4338 | UUCCCACU A UUAUCCAU | 699 | ATGCATAA GGCTAGCTACAACGA ACTGGCAA | 17456 |
| 4341 | CCACUAUU A UGCAUAUA | 701 | TATATGCA GGCTAGCTACAACGA AATACTGC | 17457 |
| 4343 | ACUAUUAU G CAUAUAUA | 7855 | TATATATG GGCTAGCTACAACGA ATAATACT | 17458 |
| 4345 | UAUUAUGC A UAUAUAAG | 5046 | CTTATATA GGCTAGCTACAACGA GCATAATA | 17459 |
| 4347 | UUAUGCAU A UAUAAGUU | 702 | AACTTATA GGCTAGCTACAACGA ATGCATAA | 17460 |
| 4349 | AUGCAUAU A UAAGUUUA | 703 | TAAACTTA GGCTAGCTACAACGA ATATGCAT | 17461 |
| 4353 | AUAUAUAA G UUUACACC | 7656 | CGTGTAAA GGCTAGCTACAACGA TTATATAT | 17462 |
| 4357 | AUAAGUUU A CACCUUUA | 707 | TAAAGGTG GGCTAGCTACAACGA AAACTTAT | 17462 |
| 4359 | AAGUUUAC A CCUUUAUC | 5047 | GATAAAGG GGCTAGCTACAACGA GTAAACTT | 17464 |
| 4365 | ACACCUUU A UCUUUCCA | 710 | TGGAAAGA GGCTAGCTACAACGA AAAGGTGT | 17465 |
| 4373 | AUCUUUCC A UGGGACCC | 5052 | GGCTCCCA GGCTAGCTACAACGA GCAAAGAT | 17466 |
| 4379 | CCAUGGGA G CCAGCUGC | 7857 | GCAGCTGG GGCTAGCTACAACGA TCCCATGG | 17467 |
| 4383 | GGGAGCCA G CUGCUUUU | 7858 | AAAAGCAG GGCTAGCTACAACGA TGGCTCCC | 17468 |
| 4386 | AGCCAGCU G CUUUUUGU | 7859 | ACAAAAAG GGCTAGCTACAACGA AGCTGGCT | 17469 |
| 4393 | UGCUUUUU G UCAUUUUU | 7860 | AAAAATCA GGCTAGCTACAACGA AAAAAGCA | 17470 |
| 4396 | UUUUUGUG A UUUUUUUA | 7861 | TAAAAAAA GGCTAGCTACAACGA CACAAAAA | 17471 |
| 4405 | UUUUUUUA A UAGUGCUU | 7862 | AAGCACTA GGCTAGCTACAACGA TAAAAAAA | 17472 |
| 4408 | UUUUAAUA G UGCUUUUU | 7863 | AAAAAGCA GGCTAGCTACAACGA TATTAAAA | 17473 |
| 4410 | UUAAUAGU G CUUUUUUU | 7864 | AAAAAAAG GGCTAGCTACAACGA ACTATTAA | 17474 |
| 4424 | UUUUUUUG A CUAACAAC | 7865 | CTTCTTAG GGCTAGCTACAACGA CAAAAAAA | 17475 |
| 4428 | UUUGACUA A CAAGAAUC | 7866 | CATTCTTC GGCTAGCTACAACGA TAGTCAAA | 17476 |
| 4434 | UAACAAGA A UGUAACUC | 7867 | GACTTACA GGCTAGCTACAACGA TCTTGTTA | 17477 |
| 4436 | ACAAGAAU G UAACUCCA | 7868 | TGGAGTTA GGCTAGCTACAACGA ATTCTTCT | 17478 |
| 4439 | AGAAUGUA A CUCCACAU | 7869 | ATCTGGAG GGCTAGCTACAACGA TACATTCT | 17479 |
| 4446 | AACUCCAC A UAGAGAAA | 7870 | TTTCTCTA GGCTAGCTACAACGA CTGCACTT | 17480 |
| 4454 | AUAGAGAA A UAGUGACA | 7871 | TGTCACTA GGCTAGCTACAACGA TTCTCTAT | 17481 |
| 4457 | GACAAAUA G UGACAACU | 7872 | ACTTCTCA GGCTAGCTACAACGA TATTTCTC | 17482 |
| 4460 | AAAUAGUG A CAAGUGAA | 7873 | TTCACTTC GGCTAGCTACAACGA CACTATTT | 17483 |
| 4464 | AGUCACAA G UCAAGAAC | 7874 | CTTCTTCA GGCTAGCTACAACGA TTGTCACT | 17484 |
| 4471 | AGUGAACA A CACUACUG | 7875 | CACTAGTG GGCTAGCTACAACGA TCTTCACT | 17485 |
| 4473 | UGAAGAAC A CUACUGCU | 5064 | AGCAGTAG GGCTAGCTACAACGA GTTCTTCA | 17486 |
| 4476 | ACAACACU A CUGCUAAA | 742 | TTTACCAC GGCTAGCTACAACGA ACTGTTCT | 17487 |
| 4479 | ACACUACU G CUAAAUCC | 7876 | GCATTTAG GGCTAGCTACAACGA ACTAGTGT | 17488 |
| 4484 | ACUGCUAA A UCCUGAUG | 7877 | CATCAGGA GGCTAGCTACAACGA TTAGCAGT | 17489 |
| 4490 | AAAUCCUC A UGUUACUC | 5070 | GAGTAACA GGCTAGCTACAACGA GACCATTT | 17490 |
| 4492 | AUCCUCAU G UGACUCAG | 7878 | CTCACTAA GGCTAGCTACAACGA ATGAGCAT | 17491 |
| 4495 | CUGAUGUU A CUCAGUCU | 747 | ACACTGAG GGCTAGCTACAACGA AACATGAG | 17492 |
| 4500 | GUUACUCA G UCUGACAG | 7879 | CTCTAACA GGCTAGCTACAACGA TGAGTAAC | 17493 |
| 4502 | UACUCAGU G UUAGAGAA | 7880 | TTCTCTAA GGCTAGCTACAACGA ACTCACTA | 17494 |
| 4511 | UUACAGAA A UCCUUCCU | 7881 | AGCAACGA GGCTAGCTACAACGA TTCTCTAA | 17495 |
| 4522 | CUUCCUAA A CCCAAUGA | 7882 | TCATTCGC GGCTAGCTACAACGA TTAGGAAG | 17496 |
| 4527 | UAAACCCA A UCACUUCC | 7883 | GGAAGTCA GGCTAGCTACAACGA TCGGTTTA | 17497 |
| 4530 | ACCCAAUG A CUUCCCUC | 7884 | CAGCGAAG GGCTAGCTACAACGA CATTGCGT | 17498 |
| 4538 | ACUUCCCU G CUCCAACC | 7885 | GGTTGCAC GGCTAGCTACAACGA AGCGAAGT | 17499 |
| 4544 | CUGCUCCA A CCCCCGCC | 7886 | GCCGCGCC GGCTAGCTACAACGA TGCAGCAG | 17500 |
| 4550 | CAACCCCC G CCACCUCA | 7887 | TCAGGTGC GGCTAGCTACAACGA GGGGCTTC | 17501 |
| 4553 | CCCCCGCC A CCUCAGCG | 5092 | CCCTCAGG GGCTAGCTACAACGA GGCCCGCG | 17502 |
| 4561 | ACCUCAGG G CACCCAGG | 7888 | CCTCCCTG GGCTAGCTACAACGA CCTGAGGT | 17503 |
| 4563 | CUCACGGC A CCCAGGAC | 5096 | GTCCTCCG GGCTAGCTACAACGA CCCCTGAC | 17504 |
| 4565 | CACGGCAC G CAGCACCA | 7889 | TCCTCCTC GGCTAGCTACAACGA CTCCCCTG | 17505 |
| 4570 | CACCCAGG A CCAGUUUC | 7890 | CAAACTGG GGCTAGCTACAACGA CCTGCGTG | 17506 |
| 4574 | CAGCACCA G UUUGAUUC | 7891 | CAATCAAA GGCTAGCTACAACGA TGCTCCTG | 17507 |
| 4579 | CCAGUUUG A UUGAGGAG | 7892 | CTCCTCAA GGCTAGCTACAACGA CAAACTGG | 17508 |
| 4587 | AUUGAGGA G CUGCACUG | 7893 | CAGTGCAG GGCTAGCTACAACGA TCCTCAAT | 17509 |
| 4590 | GAGGAGCU G CACUGAUC | 7894 | GATCAGTG GGCTAGCTACAACGA AGCTCCTC | 17510 |
| 4592 | GGAGCUGC A CUGAUCAC | 5101 | GTGATCAG GGCTAGCTACAACGA GCAGCTCC | 17511 |
| 4596 | CUGCACUG A UCACCCAA | 7895 | TTGGGTGA GGCTAGCTACAACGA CAGTGCAG | 17512 |
| 4599 | CACUGAUC A CCCAAUGC | 5103 | GCATTGGG GGCTAGCTACAACGA GATCAGTG | 17513 |
| 4604 | AUCACCCA A UCCAUCAC | 7896 | GTGATGCA GGCTAGCTACAACGA TGGGTGAT | 17514 |
| 4606 | CACCCAAU G CAUCACGU | 7897 | ACGTGATG GGCTAGCTACAACGA ATTGGGTG | 17515 |
| 4608 | CCCAAUGC A UCACGUAC | 5107 | GTACGTGA GGCTAGCTACAACGA GCATTGGG | 17516 |
| 4611 | AAUGCAUC A CGUACCCC | 5108 | GGGGTACG GGCTAGCTACAACGA GATGCATT | 17517 |
| 4613 | UGCAUCAC G UACCCCAC | 7898 | GTGGGGTA GGCTAGCTACAACGA GTGATGCA | 17518 |
| 4615 | CAUCACGU A CCCCACUG | 764 | CAGTGGGG GGCTAGCTACAACGA ACGTGATG | 17519 |
| 4620 | CGUACCCC A CUGGGCCA | 5112 | TGGCCCAG GGCTAGCTACAACGA GGGGTACG | 17520 |
| 4625 | CCCACUGG G CCAGCCCU | 7899 | AGGGCTGG GGCTAGCTACAACGA CCAGTGGG | 17521 |
| 4629 | CUGGGCCA G CCCUGCAG | 7900 | CTGCAGGG GGCTAGCTACAACGA TGGCCCAG | 17522 |
| 4634 | CCAGCCCU G CAGCCCAA | 7901 | TTGGGCTG GGCTAGCTACAACGA AGGUCTGG | 17523 |
| 4637 | GCCCUGCA G CCCAAAAC | 7902 | GTTTTGGG GGCTAGCTACAACGA TGCAGGGC | 17524 |
| 4644 | AGCCCAAA A CCCAGGGC | 7903 | GCCCTGGG GGCTAGCTACAACGA TTTGGGCT | 17525 |
| 4651 | AACCCAGG G CAACAAGC | 7904 | GCTTGTTG GGCTAGCTACAACGA CCTGGGTT | 17526 |
| 4654 | CCAGGGCA A CAAGCCCG | 7905 | CGGGCTTG GGCTAGCTACAACGA TGCCCTGG | 17527 |
| 4658 | GGCAACAA G CCCGUUAG | 7906 | CTAACGGG GGCTAGCTACAACGA TTGTTGCC | 17528 |
| 4662 | ACAAGCCC G UUAGCCCC | 7907 | GGGGCTAA GGCTAGCTACAACGA GGGCTTGT | 17529 |
| 4666 | GCCCGUUA G CCCCAGGG | 7908 | CCCTGGGG GGCTAGCTACAACGA TAACGGGC | 17530 |
| 4676 | CCCAGGGG A UCACUGGC | 7909 | GCCAGTGA GGCTAGCTACAACGA CCCCTGGG | 17531 |
| 4679 | AGGGGAUC A CUGGCUGG | 5134 | CCAGCCAG GGCTAGCTACAACGA GATCCCCT | 17532 |
| 4683 | GAUCACUG G CUGGCCUG | 7910 | CAGGCCAG GGCTAGCTACAACGA CAGTGATC | 17533 |
| 4687 | ACUGGCUG G CCUGAGCA | 7911 | TGCTCAGG GGCTAGCTACAACGA CAGCCAGT | 17534 |
| 4693 | UGGCCUGA G CAACAUCU | 7912 | AGATGTTG GGCTAGCTACAACGA TCAGGCCA | 17535 |
| 4696 | CCUGAGCA A CAUCUCGG | 7913 | CCGAGATG GGCTAGCTACAACGA TGCTCAGG | 17536 |
| 4698 | UGAGCAAC A UCUCGGGA | 5140 | TCCCGAGA GGCTAGCTACAACGA GTTGCTCA | 17537 |
| 4707 | UCUCGGGA G UCCUCUAG | 7914 | CTAGAGGA GGCTAGCTACAACGA TCCCGAGA | 17538 |
| 4715 | GUCCUCUA G CAGGCCUA | 7915 | TAGGCCTG GGCTAGCTACAACGA TAGAGGAC | 17539 |
| 4719 | UCUAGCAG G CCUAAGAC | 7916 | GTCTTAGG GGCTAGCTACAACGA CTGCTAGA | 17540 |
| 4726 | GGCCUAAG A CAUGUGAG | 7917 | CTCACATG GGCTAGCTACAACGA CTTAGGCC | 17541 |
| 4728 | CCUAAGAC A UGUGAGGA | 5148 | TCCTCACA GGCTAGCTACAACGA GTCTTAGG | 17542 |
| 4730 | UAAGACAU G UGAGGAGG | 7918 | CCTCCTCA GGCTAGCTACAACGA ATGTCTTA | 17543 |
| 4752 | GAAAAAAA G CAAAAAGC | 7919 | GCTTTTTG GGCTAGCTACAACGA TTTTTTTC | 17544 |
| 4759 | AGCAAAAA G CAAGGGAG | 7920 | CTCCCTTG GGCTAGCTACAACGA TTTTTGCT | 17545 |
| 4777 | AAAGAGAA A CCGGGAGA | 7921 | TCTCCCGG GGCTAGCTACAACGA TTCTCTTT | 17546 |
| 4788 | GGGAGAAG G CAUGAGAA | 7922 | TTCTCATG GGCTAGCTACAACGA CTTCTCCC | 17547 |
| 4790 | GAGAAGGC A UGAGAAAG | 5152 | CTTTCTCA GGCTAGCTACAACGA GCCTTCTC | 17548 |
| 4800 | GAGAAAGA A UUUGAGAC | 7923 | GTCTCAAA GGCTAGCTACAACGA TCTTTCTC | 17549 |
| 4807 | AAUUUGAG A CGCACCAU | 7924 | ATGGTGCG GGCTAGCTACAACGA CTCAAATT | 17550 |
| 4809 | UUUGAGAC G CACCAUGU | 7925 | ACATGGTG GGCTAGCTACAACGA GTCTCAAA | 17551 |
| 4811 | UGAGACGC A CCAUGUGG | 5153 | CCACATGG GGCTAGCTACAACGA GCGTCTCA | 17552 |
| 4814 | GACGCACC A UGUGGGCA | 5155 | TGCCCACA GGCTAGCTACAACGA GGTGCGTC | 17553 |
| 4816 | CGCACCAU G UGGGCACG | 7926 | CGTGCCCA GGCTAGCTACAACGA ATGGTGCG | 17554 |
| 4820 | CCAUGUGG G CACGGAGG | 7927 | CCTCCGTG GGCTAGCTACAACGA CCACATGG | 17555 |
| 4822 | AUGUGGGC A CGGAGGGG | 5156 | CCCCTCCG GGCTAGCTACAACGA GCCCACAT | 17556 |
| 4832 | GGAGGGGG A CGGGGCUC | 7928 | GAGCCCCG GGCTAGCTACAACGA CCCCCTCC | 17557 |
| 4837 | GGGACGGG G CUCAGCAA | 7929 | TTGCTGAG GGCTAGCTACAACGA CCCGTCCC | 17558 |
| 4842 | GGGGCUCA G CAAUGCCA | 7930 | TGGCATTG GGCTAGCTACAACGA TGAGCCCC | 17559 |
| 4845 | GCUCAGCA A UGCCAUUU | 7931 | AAATGGCA GGCTAGCTACAACGA TGCTGAGC | 17560 |
| 4847 | UCAGCAAU G CCAUUUCA | 7932 | TGAAATGG GGCTAGCTACAACGA ATTGCTGA | 17561 |
| 4850 | GCAAUGCC A UUUCAGUG | 5161 | CACTGAAA GGCTAGCTACAACGA GGCATTGC | 17562 |
| 4856 | CCAUUUCA G UGCCUUCC | 7933 | GGAACCCA GGCTAGCTACAACGA TGAAATGG | 17563 |
| 4859 | UUUCAGUC G CUUCCCAG | 7934 | CTGGGAAG GGCTAGCTACAACGA CACTGAAA | 17564 |
| 4867 | GCUUCCCA G CUCUGACC | 7935 | GGTCAGAG GGCTAGCTACAACGA TGGGAAGC | 17565 |
| 4873 | CAGCUCUG A CCCUUCUA | 7936 | TAGAAGGG GGCTAGCTACAACGA CAGAGCTG | 17566 |
| 4881 | ACCCUUCU A CAUUUCAG | 785 | CTCAAATG GGCTAGCTACAACGA AGAAGGGT | 17567 |
| 4883 | CCUUCUAC A UUUGAGGG | 5173 | CCCTCAAA GGCTAGCTACAACGA GTACAAGG | 17568 |
| 4891 | AUUUGAGG G CCCAGCCA | 7937 | TGGCTGGG GGCTAGCTACAACGA CCTCAAAT | 17569 |
| 4896 | AGGGCCCA G CCAGGAGC | 7938 | GCTCCTGG GGCTAGCTACAACGA TGGGCCCT | 17570 |
| 4903 | AGCCAGGA G CAGAUGGA | 7939 | TCCATCTG GGCTAGCTACAACGA TCCTGGCT | 17571 |
| 4907 | AGGAGCAG A UGGACAGC | 7940 | GCTGTCCA GGCTAGCTACAACGA CTGCTCCT | 17572 |
| 4911 | GCAGAUGG A CAGCGAUG | 7941 | CATCGCTG GGCTAGCTACAACGA CCATCTGC | 17573 |
| 4914 | GAUGGACA G CGAUGAGG | 7942 | CCTCATCG GGCTAGCTACAACGA TGTCCATC | 17574 |
| 4917 | GGACAGCG A UGAGGGGA | 7943 | TCCCCTCA GGCTAGCTACAACGA CGCTGTCC | 17575 |
| 4925 | AUGAGGUG A CAUUUUCU | 7944 | AGAAAATG GGCTAGCTACAACGA CCCCTCAT | 17576 |
| 4927 | GAGGGGAC A UUUUCUGG | 5181 | CCAGAAAA GGCTAGCTACAACGA GTCCCCTC | 17577 |
| 4936 | UUUUCUGG A UUCUGGGA | 7945 | TCCCAGAA GGCTAGCTACAACGA CCAGAAAA | 17578 |
| 4946 | UCUGGGAG G CAAGAAAA | 7946 | TTTTCTTG GGCTAGCTACAACGA CTCCCAGA | 17579 |
| 4957 | AGAAAAGG A CAAAUAUC | 7947 | GATATTTG GGCTAGCTACAACGA CCTTTTCT | 17580 |
| 4961 | AAGGACAA A UAUCUUUU | 7948 | AAAAGATA GGCTAGCTACAACGA TTGTCCTT | 17581 |
| 4963 | GGACAAAU A UCUUUUUU | 794 | AAAAAAGA GGCTAGCTACAACGA ATTTGTCC | 17582 |
| 4975 | UUUUUGGA A CUAAAGCA | 7949 | TGCTTTAG GGCTAGCTACAACGA TCCAAAAA | 17583 |
| 4981 | GAACUAAA G CAAAUUUU | 7950 | AAAATTTG GGCTAGCTACAACGA TTTAGTTC | 17584 |
| 4985 | UAAAGCAA A UUUUAGAC | 7951 | GTCTAAAA GGCTAGCTACAACGA TTGCTTTA | 17585 |
| 4992 | AAUUUUAG A CCUUUACC | 7952 | GGTAAAGG GGCTAGCTACAACGA CTAAAATT | 17586 |
| 4998 | AGACCUUU A CCUAUGGA | 808 | TCCATAGG GGCTAGCTACAACGA AAAGGTCT | 17587 |
| 5002 | CUUUACCU A UGGAAGUG | 809 | CACTTCCA GGCTAGCTACAACGA AGGTAAAG | 17588 |
| 5008 | CUAUGGAA G UGGUUCUA | 7953 | TAGAACCA GGCTAGCTACAACGA TTCCATAG | 17589 |
| 5011 | UGGAAGUG G UUCUAUGU | 7954 | ACATAGAA GGCTAGCTACAACGA CACTTCCA | 17590 |
| 5016 | GUGGUUCU A UGUCCAUU | 812 | AATGGACA GGCTAGCTACAACGA AGAACCAC | 17591 |
| 5018 | GGUUCUAU G UCCAUUCU | 7955 | AGAATGGA GGCTAGCTACAACGA ATAGAACC | 17592 |
| 5022 | CUAUGUCC A UUCUCAUU | 5195 | AATGAGAA GGCTAGCTACAACGA GGACATAG | 17593 |
| 5028 | CCAUUCUC A UUCGUGGC | 5197 | GCCACGAA GGCTAGCTACAACGA GAGAATGG | 17594 |
| 5032 | UCUCAUUC G UGGCAUGU | 7956 | ACATGCCA GGCTAGCTACAACGA GAATGAGA | 17595 |
| 5035 | CAUUCGUG G CAUGUUUU | 7957 | AAAACATG GGCTAGCTACAACGA CACGAATG | 17596 |
| 5037 | UUCGUGGC A UGUUUUGA | 5198 | TCAAAACA GGCTAGCTACAACGA GCCACGAA | 17597 |
| 5039 | CGUGGCAU G UUUUGAUU | 7958 | AATCAAAA GGCTAGCTACAACGA ATGCCACG | 17598 |
| 5045 | AUGUUUUG A UUUGUAGC | 7959 | GCTACAAA GGCTAGCTACAACGA CAAAACAT | 17599 |
| 5049 | UUUGAUUU G UAGCACUG | 7960 | CAGTGCTA GGCTAGCTACAACGA AAATCAAA | 17600 |
| 5052 | GAUUUGUA G CACUGAGG | 7961 | CCTCAGTG GGCTAGCTACAACGA TACAAATC | 17601 |
| 5054 | UUUGUAGC A CUGAGGCU | 5199 | ACCCTCAG GGCTAGCTACAACGA GCTACAAA | 17602 |
| 5061 | CACUGAGC G UGCCACUC | 7962 | GACTGCCA GGCTAGCTACAACGA CCTCACTG | 17603 |
| 5064 | UCACGGUC G CACUCAAC | 7963 | CTTGACTG GGCTAGCTACAACGA CACCCTCA | 17604 |
| 5066 | ACCCUCCC A CUCAACUC | 5201 | CACTTGAG GGCTAGCTACAACGA CCCACCCT | 17605 |
| 5071 | GGCACUCA A CUCUGAGC | 7964 | GCTCAGAG GGCTAGCTACAACGA TGAGTGCC | 17606 |
| 5078 | AACUCUGA G CCCAUACU | 7965 | AGTATGGG GGCTAGCTACAACGA TCAGAGTT | 17607 |
| 5082 | CUGAGCCC A UACUUUUG | 5208 | CAAAACTA GGCTAGCTACAACGA GGGCTCAG | 17608 |
| 5084 | GACCCCAU A CUUUUGGC | 827 | GCCAAAAC GGCTAGCTACAACGA ATGGGCTC | 17609 |
| 5091 | UACUUUUG G CUCCUCUA | 7966 | TAGAGGAG GGCTAGCTACAACGA CAAAAGTA | 17610 |
| 5100 | CUCCUCUA G UAAGAUGC | 7967 | GCATCTTA GGCTAGCTACAACGA TAGAGGAG | 17611 |
| 5105 | CUAGUAAG A UGCACUGA | 7968 | TCAGTGCA GGCTAGCTACAACGA CTTACTAG | 17612 |
| 5107 | AGUAAGAU G CACUGAAA | 7969 | TTTCAGTG GGCTAGCTACAACGA ATCTTACT | 17613 |
| 5109 | UAAGAUGC A CUGAAAAC | 5214 | GTTTTCAG GGCTAGCTACAACGA GCATCTTA | 17614 |
| 5116 | CACUGAAA A CUUAGCCA | 7970 | TGGCTAAG GGCTAGCTACAACGA TTTCAGTG | 17615 |
| 5121 | AAAACUUA G CCAGAGUU | 7971 | AACTCTGG GGCTAGCTACAACGA TAAGTTTT | 17616 |
| 5127 | UAGCCAGA G UUACGUUG | 7972 | CAACCTAA GGCTAGCTACAACGA TCTCGCTA | 17617 |
| 5132 | AGAGUUAG G UUGUCUCC | 7973 | GGAGACAA GGCTAGCTACAACGA CTAACTCT | 17618 |
| 5135 | GUUAGGUU G UCUCCAGG | 7974 | CCTGGAGA GGCTAGCTACAACGA AACCTAAC | 17619 |
| 5143 | GUCUCCAG G CCAUGAUG | 7975 | CATCATGG GGCTAGCTACAACGA CTGGAGAC | 17620 |
| 5146 | UCCAGGCC A UGAUGGGC | 5223 | GGCCATCA GGCTAGCTACAACGA GGCCTGGA | 17621 |
| 5149 | AGGCCAUG A UGGCCUUA | 7976 | TAAGGCCA GGCTAGCTACAACGA CATGGCCT | 17622 |
| 5152 | CCAUGAUG G CCUUACAC | 7977 | GTGTAAGG GGCTAGCTACAACGA CATCATGG | 17623 |
| 5157 | AUGGCCUU A CACUGAAA | 843 | TTTCAGTG GGCTAGCTACAACGA AAGGCCAT | 17624 |
| 5159 | GGCCUUAC A CUGAAAAU | 5226 | ATTTTCAG GGCTAGCTACAACGA GTAAGGCC | 17625 |
| 5166 | CACUGAAA A UGUCACAU | 7978 | ATGTGACA GGCTAGCTACAACGA TTTCAGTG | 17626 |
| 5168 | CUGAAAAU G UCACAUUC | 7979 | GAATGTGA GGCTAGCTACAACGA ATTTTCAG | 17627 |
| 5171 | AAAAUGUC A CAUUCUAU | 5228 | ATAGAATG GGCTAGCTACAACGA GACATTTT | 17628 |
| 5173 | AAUGUCAC A UUCUAUUU | 5229 | AAATAGAA GGCTAGCTACAACGA GTGACATT | 17629 |
| 5178 | CACAUUCU A UUUUGGGU | 847 | ACCCAAAA GGCTAGCTACAACGA AGAATGTG | 17630 |
| 5185 | UAUUUUGG G UAUUAAUA | 7980 | TATTAATA GGCTAGCTACAACGA CCAAAATA | 17631 |
| 5187 | UUUUGGGU A UUAAUAUA | 851 | TATATTAA GGCTAGCTACAACGA ACCCAAAA | 17632 |
| 5191 | GGGUAUUA A UAUAUAGU | 7981 | ACTATATA GGCTAGCTACAACGA TAATACCC | 17633 |
| 5193 | GUAUUAAU A UAUAGUCC | 854 | GGACTATA GGCTAGCTACAACGA ATTAATAC | 17634 |
| 5195 | AUUAAUAU A UAGUCCAG | 855 | CTGGACTA GGCTAGCTACAACGA ATATTAAT | 17635 |
| 5198 | AAUAUAUA G UCCAGACA | 7982 | TCTCTGGA GGCTAGCTACAACGA TATATATT | 17636 |
| 5204 | UAGUCCAG A CACUUAAC | 7983 | GTTAACTG GGCTAGCTACAACGA CTGGACTA | 17637 |
| 5206 | GUCCAGAC A CUUAACUC | 5233 | GAGTTAAG GGCTAGCTACAACGA GTCTGGAC | 17638 |
| 5211 | GACACUUA A CUCAAUUU | 7984 | AAATTGAG GGCTAGCTACAACGA TAAGTGTC | 17639 |
| 5216 | UUAACUCA A UUUCUUGG | 7985 | CCAAGAAA GGCTAGCTACAACGA TGAGTTAA | 17640 |
| 5224 | AUUUCUUG G UAUUAUUC | 7986 | GAATAATA GGCTAGCTACAACGA CAAGAAAT | 17641 |
| 5226 | UUCUUCGU A UUAUUCUG | 865 | CAGAATAA GGCTAGCTACAACGA ACCAACAA | 17642 |
| 5229 | UUGGUAUU A UUCUGUUU | 867 | AAACAGAA GGCTAGCTACAACGA AATACCAA | 17643 |
| 5234 | AUUAUUCU G UUUUGCAC | 7987 | GTGCAAAA GGCTAGCTACAACGA AGAATAAT | 17644 |
| 5239 | UCUGUUUU G CACAGUUA | 7988 | TAACTGTG GGCTAGCTACAACGA AAAACAGA | 17645 |
| 5241 | UGUUUUGC A CAGUUAGU | 5239 | ACTAACTG GGCTAGCTACAACGA GCAAAACA | 17646 |
| 5244 | UUUGCACA G UUAGUUCU | 7989 | ACAACTAA GGCTAGCTACAACGA TGTGCAAA | 17647 |
| 5248 | CACAGUUA G UUGUGAAA | 7990 | TTTCACAA GGCTAGCTACAACGA TAACTGTC | 17648 |
| 5251 | ACUUACUU G UCAAACAA | 7991 | TTCTTTCA GGCTAGCTACAACGA AACTAACT | 17649 |
| 5261 | CAAACAAA G CUGACAAC | 7992 | CTTCTCAC CCCTACCTACAACCA TTTCTTTC | 17650 |
| 5271 | UCACAACA A UCAAAAUC | 7993 | CATTTTCA GGCTAGCTACAACGA TCTTCTCA | 17651 |
| 5277 | CAAUGAAA A UCCACUCC | 7994 | CCACTGCA GGCTAGCTACAACGA TTTCATTC | 17652 |
| 5279 | AUGAAAAU G CACUCCUC | 7995 | CACCACTG GGCTAGCTACAACGA ATTTTCAT | 17653 |
| 5282 | AAAAUCCA G UCCUCAGG | 7996 | CCTCACCA GGCTAGCTACAACGA TGCATTTT | 17654 |
| 5294 | UGAGGAGA G UUUUCUCC | 7997 | GGAGAAAA GGCTAGCTACAACGA TCTCCTCA | 17655 |
| 5303 | UUUUCUCC A UAUCAAAA | 5247 | TTTTGATA GGCTAGCTACAACGA GGAGAAAA | 17656 |
| 5305 | UUCUCCAU A UCAAAACG | 882 | CGTTTTGA GGCTAGCTACAACGA ATGGAGAA | 17657 |
| 5311 | AUAUCAAA A CGAGGGCU | 7998 | AGCCCTCG GGCTAGCTACAACGA TTTGAEAT | 17658 |
| 5317 | AAACGAGG G CUGAUGGA | 7999 | TCCATCAG GGCTAGCTACAACGA CCTCGTTT | 17659 |
| 5321 | GAGGGCUG A UGGAGGAA | 8000 | TTCCTCCA GGCTAGCTACAACGA CAGCCCTC | 17660 |
| 5334 | GGAAAAAG G UCAAUAAG | 8001 | CTTATTGA GGCTAGCTACAACGA CTTTTTCC | 17661 |
| 5338 | AAAGGUCA A UAAGGUCA | 8002 | TGACCTTA GGCTAGCTACAACGA TGACCTTT | 17662 |
| 5343 | UCAAUAAG G UCAAGGGA | 8003 | TCCCTTGA GGCTAGCTACAACGA CTTATTGA | 17663 |
| 5354 | AAGGGAAG A CCCCGUCU | 8004 | AGACGGGG GGCTAGCTACAACGA CTTCCCTT | 17664 |
| 5359 | AAGACCCC G UCUCUAAA | 8005 | TATAGAGA GGCTAGCTACAACGA GGGGTCTT | 17665 |
| 5365 | CCGUCUCU A UACCAACC | 889 | GGTTGGTA GGCTAGCTACAACGA AGAGACGG | 17666 |
| 5367 | GUCUCUAU A CCAACCAA | 890 | TTGGTTGG GGCTAGCTACAACGA ATAGAGAC | 17667 |
| 5371 | CUAUACCA A CCAAACCA | 8006 | TGGTTTGG GGCTAGCTACAACGA TGGTATAG | 17668 |
| 5376 | CCAACCAA A CCAAUUCA | 8007 | TGAATTGG GGCTAGCTACAACGA TTGGTTGG | 17669 |
| 5380 | CCAAACCA A UUCACCAA | 8008 | TTGGTGAA GGCTAGCTACAACGA TGGTTTGG | 17670 |
| 5384 | ACCAAUUC A CCAACACA | 5263 | TGTGTTGG GGCTAGCTACAACGA GAATTGGT | 17671 |
| 5388 | AUUCACCA A CACAGUUG | 8009 | CAACTGTG GGCTAGCTACAACGA TGGTGAAT | 17672 |
| 5390 | UCACCAAC A CAGUUGGG | 5266 | CCCAACTG GGCTAGCTACAACGA GTTGGTGA | 17673 |
| 5393 | CCAACACA G UUGGGACC | 8010 | GGTCCCAA GGCTAGCTACAACGA TGTGTTGG | 17674 |
| 5399 | CAGUUGGG A CCCAAAAC | 8011 | GTTTTGGG GGCTAGCTACAACGA CCCAACTG | 17675 |
| 5406 | GACCCAAA A CACAGGAA | 8012 | TTCCTGTG GGCTAGCTACAACGA TTTGGGTC | 17676 |
| 5408 | CCCAAAAC A CAGGAAGU | 5271 | ACTTCCTG GGCTAGCTACAACGA GTTTTGGG | 17677 |
| 5415 | CACAGGAA G UCAGUCAC | 8013 | GTGACTGA GGCTAGCTACAACGA TTCCTGTG | 17678 |
| 5419 | GGAAGUCA G UCACGUUU | 8014 | AAACGTGA GGCTAGCTACAACGA TGACTTCC | 17679 |
| 5422 | AGUCAGUC A CGUUUCCU | 5274 | AGGAAACG GGCTAGCTACAACGA GACTUACT | 17680 |
| 5424 | UCAGUCAC G UDUCCUUG | 8015 | AAAGGAAA GGCTAGCTACAACGA GTGACTGA | 17681 |
| 5435 | UCCUUUUC A UUUAAUGG | 5277 | CCATTAAA GGCTAGCTACAACGA GAAAAGGA | 17682 |
| 5440 | UUCAUUUA A UGGGGAUU | 8016 | AATCCCCA GGCTAGCTACAACGA TAAATGAA | 17683 |
| 5446 | UAAUGGGG A UUCCACUA | 8017 | TAGTGGAA GGCTAGCTACAACGA CCCCATTA | 17684 |
| 5451 | GGGAUUCC A CUAUCUCA | 5279 | TGAGATAG GGCTAGCTACAACGA GGAATCCC | 17685 |
| 5454 | AUUCCACU A UCUCACAC | 908 | GTGTUAGA GGCTAGCTACAACGA AGTGGAAT | 17686 |
| 5459 | ACUAUCUC A CACUAAUC | 5282 | GATTAGTG GGCTAGCTACAACGA GAGATAGT | 17687 |
| 5461 | UAUCUCAC A CUAAUCUG | 5283 | CAGATTAG GGCTAGCTACAACGA GTGAGATA | 17688 |
| 5465 | UCACACUA A UCUGAAAG | 8018 | CTTTCAGA GGCTAGCTACAACGA TAGTGTGA | 17689 |
| 5475 | CUGAAAGG A UGUGGAAG | 8019 | CTTCCACA GGCTAGCTACAACGA CCTTTCAG | 17690 |
| 5477 | GAAAGGAU G UGGAAGAG | 8020 | CTCTTCCA GGCTAGCTACAACGA ATCCTTTC | 17691 |
| 5485 | GUGGAAGA G CAUDAGCU | 8021 | AGCTAATG GGCTAGCTACAACGA TCTTCCAC | 17692 |
| 5487 | GGAAGAGC A UUAGCUGG | 5286 | CCAGCTAA GGCTAGCTACAACGA GCTCTTCC | 17693 |
| 5491 | GAUCAUDA G CUGUCUCA | 8022 | TGCGCCAG GGCTAGCTACAACGA TAATGCTC | 17694 |
| 5495 | AUDAUCUG G CGCAUAUU | 8023 | AATATGCG GGCTAGCTACAACGA CAGCTAAT | 17695 |
| 5497 | GAUCUGUC G CAUAUUAA | 8024 | TTAATATG GGCTAGCTACAACGA GCCAGCTA | 17696 |
| 5499 | UCUGUCUC A UAUUAAGC | 5288 | GCTTAATA GGCTAGCTACAACUA GCGCCAGC | 17697 |
| 5501 | UGUCUCAU A UUAAGCAC | 915 | GTGCTTAA GGCTAUCTACAACGA ATGCGCCA | 17698 |
| 5506 | CAUAUUAA G CACUUUAA | 8025 | TTAAAGTG GGCTAGCTACAACGA TTAATATG | 17699 |
| 5508 | UAUUAAUC A CUUUAAGC | 5289 | GCTTAAAG GGCTAGCTACAACGA GCTTAATA | 17700 |
| 5515 | CACUUUAA G CUCCUUGA | 8026 | TCAAGGAG GGCTAGCTACAACGA TTAAAGTG | 17701 |
| 5524 | CUCCUUGA G UAAAAAGG | 8027 | CCTTTTTA GGCTAGCTACAACGA TCAAGGAG | 17702 |
| 5532 | UUAAAAAG G UGGUAUUU | 8028 | ACATACCA GGCTAGCTACAACGA CTTTTTAC | 17703 |
| 5535 | AAAAGGUG G UAUGUAAU | 8029 | ATTACATA GGCTAGCTACAACGA CACCTTTT | 17704 |
| 5537 | AACGUGGU A UGUAAUUU | 924 | AAATTACA GGCTAGCTACAACGA ACCACCTT | 17705 |
| 5539 | GGUGGUAU G UAAUUUAU | 8030 | ATAAATTA GGCTAGCTACAACGA ATACCACC | 17706 |
| 5542 | GGUAUGUA A UUUAUGCA | 8031 | TGCATAAA GGCTAGCTACAACGA TACATACC | 17707 |
| 5546 | UGUAAUUU A UGCAAGGU | 928 | ACCTTGCA GGCTAGCTACAACGA AAATTACA | 17708 |
| 5548 | UAAUUUAU G CAAGGUAU | 8032 | ATACCTTG GGCTAGCTACAACGA ATAAATTA | 17709 |
| 5553 | UAUGCAAG G UAUUUCUC | 8033 | GAGAAATA GGCTAGCTACAACGA CTTGCATA | 17710 |
| 5555 | UGCAAGGU A UUUCUCCA | 929 | TGGAGAAA GGCTAGCTACAACGA ACCTTGCA | 17711 |
| 5564 | UUUCUCCA G UUGCGACU | 8034 | AGTCCCAA GGCTAGCTACAACGA TGGACAAA | 17712 |
| 5570 | CAGUUGGG A CUCAGGAU | 8035 | ATCCTGAG GGCTAGCTACAACGA CCCAACTG | 17713 |
| 5577 | GACUCAGG A UAUUAGUU | 8036 | AACTAATA GGCTAGCTACAACGA CCTGAGTC | 17714 |
| 5579 | CUCAGCAU A UUAGUUAA | 936 | TTAACTAA GGCTAGCTACAACGA ATCCTGAG | 17715 |
| 5583 | GGAUAUUA G UUAAUCAC | 8037 | CTCATTAA GGCTAGCTACAACGA TAATATCC | 17716 |
| 5587 | AUUAGUUA A UCAGCCAU | 8038 | ATGCCTCA GGCTAGCTACAACGA TAACTAAT | 17717 |
| 5591 | GUUAAUGA G CCAUCACU | 8039 | AGTGATGG GGCTAGCTACAACGA TCATTAAC | 17718 |
| 5594 | AAUCAGCC A UCACUACA | 5301 | TCTAGTCA GGCTAGCTACAACGA GGCTCATT | 17719 |
| 5597 | GACCCAUC A CUACAAGA | 5302 | TCTTCTAG GGCTAGCTACAACGA GATGGCTC | 17720 |
| 5609 | CAACAAAA G CCCAUUUU | 8040 | AAAATGGG GGCTAGCTACAACGA TTTTCTTC | 17721 |
| 5613 | AAAAGCCC A UUUUCAAC | 5306 | GTTCAAAA GGCTAGCTACAACGA GCGCTTTT | 17722 |
| 5620 | CAUUUUCA A CUGCUUUC | 8041 | CAAAGCAC GGCTAGCTACAACGA TGAAAATG | 17723 |
| 5623 | UUUCAACU G CUUUGAAA | 8042 | TTTCAAAG GGCTAGCTACAACGA AGTTCAAA | 17724 |
| 5631 | GCUUUGAA A CUUGCCUG | 8043 | CAGCCAAG GGCTAGCTACAACGA TTCAAAGC | 17725 |
| 5635 | UGAAACUU G CCUCGGGU | 8044 | ACCCCAGG GGCTAGCTACAACGA AACTTTCA | 17726 |
| 5642 | UCCCUGCG G UCUGACCA | 8045 | TGCTCAGA GGCTAGCTACAACGA CCCACCCA | 17727 |
| 5648 | GGGUCUGA G CAUGAUCG | 8046 | CCATCATG GGCTAGCTACAACGA TCAGACCC | 17728 |
| 5650 | GUCUGAUC A UGAUGUGA | 5314 | TCCCATCA GGCTAGCTACAACGA GCTCAGAC | 17729 |
| 5653 | UGAGCAUG A UGGGAAUA | 8047 | TATTCCCA GGCTAGCTACAACGA CATGCTCA | 17730 |
| 5659 | UGAUGUCA A UACGGAGA | 8048 | TCTCCCTA GGCTAGCTACAACGA TCCCATCA | 17731 |
| 5667 | AUAGGCAG A CAGGGUAG | 8049 | CTACCCTG GGCTAGCTACAACGA CTCCCTAT | 17732 |
| 5672 | CACACAGG G UAGGAAAC | 8050 | CTTTCCTA GGCTAGCTACAACGA CCTCTCTC | 17733 |
| 5682 | AGGAAAGG G CGCCUACU | 8051 | AGTAGGCG GGCTAGCTACAACGA CCTTTCCT | 17734 |
| 5684 | GAAAGGGC G CCUACUCU | 8052 | AGAGTAGG GGCTAGCTACAACGA GCCCTTTC | 17735 |
| 5688 | GGGCGCCU A CUCUUCAG | 953 | CTCAAGAG GGCTAGCTACAACGA AGGCGCCC | 17736 |
| 5698 | UCUUCAGG G UCUAAACA | 8053 | TCTTTAGA GGCTAGCTACAACGA CCTGAAGA | 17737 |
| 5706 | GUCUAAAG A UCAAGUCC | 8054 | CCACTTGA GGCTAGCTACAACGA CTTTAGAC | 17738 |
| 5711 | AAGAUCAA G UGGGCCUU | 8055 | AACGCCCA GGCTAGCTACAACGA TTGATCTT | 17739 |
| 5715 | UCAAGUCC G CCUUGGAU | 8056 | ATCCAAGG GGCTAGCTACAACGA CCACTTCA | 17740 |
| 5722 | GGCCUUGG A UCGCUAAC | 8057 | CTTAGCGA GGCTAGCTACAACGA CCAAGCCC | 17741 |
| 5725 | CUUGGAUC G CUAAGCUG | 8058 | CAGCTTAG GGCTAGCTACAACGA GATCCAAC | 17742 |
| 5730 | AUCCCUAA G CUGGCUCU | 8059 | AGACCCAG GGCTAGCTACAACGA TTAGCCAT | 17743 |
| 5734 | CUAACCUG G CUCUGUUU | 8060 | AAACAGAG GGCTAGCTACAACGA CACCTTAC | 17744 |
| 5739 | CUCCCUCU G UUUGAUGC | 8061 | GCATCAAA GGCTAGCTACAACGA AGAGCCAG | 17745 |
| 5744 | UCUGUUUC A UCCUAUUU | 8062 | AAATACCA GGCTAGCTACAACGA CAAACACA | 17746 |
| 5746 | UGUUUCAU G CUAUUUAU | 8063 | ATAAATAG GGCTAGCTACAACGA ATCAAACA | 17747 |
| 5749 | UUGAUCCU A UUUAUCCA | 966 | TCCATAAA GGCTAGCTACAACGA AGCATCAA | 17748 |
| 5753 | UCCUAUUU A UCCAACUU | 969 | AACTTGCA GGCTAGCTACAACGA AAATACCA | 17749 |
| 5755 | CUAUUUAU G CAACUUAG | 8064 | CTAACTTC GGCTAGCTACAACGA ATAAATAC | 17750 |
| 5759 | UUAUGCAA G UUACGGUC | 8065 | GACCCTAA GGCTAGCTACAACGA TTGCATAA | 17751 |
| 5765 | AACUUAGG G UCUAUCUA | 8066 | TACATACA GGCTAGCTACAACGA CCTAACTT | 17752 |
| 5769 | UAGGGUCU A UGUAUUUA | 973 | TAAATACA GGCTAGCTACAACGA AGACCCTA | 17753 |
| 5771 | GGGUCUAU G UAUUUAGG | 8067 | CCTAAATA GGCTAGCTACAACGA ATAGACCC | 17754 |
| 5773 | CUCUAUGU A UUUAGGAU | 974 | ATCCTAAA GGCTAGCTACAACGA ACATAGAC | 17755 |
| 5780 | UAUUUAGG A UGCGCCUA | 8068 | TAGGCGCA GGCTAGCTACAACGA CCTAAATA | 17756 |
| 5782 | UUUAGGAU G CGCCUACU | 8069 | AGTAGGCC GGCTAGCTACAACGA ATCCTAAA | 17757 |
| 5784 | UAGGAUGC G CCUACUCU | 8070 | AGAGTAGG GGCTAGCTACAACGA GCATCCTA | 17758 |
| 5788 | AUCCGCCU A CUCUUCAC | 978 | CTGAAGAG GGCTAGCTACAACGA AGCCGCAT | 17759 |
| 5798 | UCUUCAGG G UCUAAAGA | 8053 | TCTTTAGA GGCTAGCTACAACGA CCTGAAGA | 17737 |
| 5806 | CUCUAAAG A UCAACUGG | 8054 | CCACTTCA GGCTAGCTACAACGA CTTTAGAC | 17738 |
| 5811 | AACAUCAA G UGGGCCUU | 8055 | AAGGCCCA GGCTAGCTACAACGA TTCATCTT | 17739 |
| 5815 | UCAAGUGG G CCUUCCAU | 8056 | ATCCAACG GGCTAGCTACAACGA CCACTTGA | 17740 |
| 5822 | GGCCUUGC A UCGCUAAC | 8057 | CTTACCCA GGCTAGCTACAACGA CCAACCCC | 17741 |
| 5825 | CUUGCAUC G CUAAGCUC | 8058 | CAGCTTAC GGCTAGCTACAACGA CATCCAAG | 17742 |
| 5830 | AUCGCUAA G CUGCCUCU | 8059 | ACACCCAG GGCTAGCTACAACGA TTACCCAT | 17743 |
| 5834 | CUAAGCUC G CUCUCUUU | 8060 | AAACAGAG GGCTAGCTACAACGA CAGCTTAC | 17744 |
| 5839 | CUCCCUCU G UUUGAUCC | 8061 | GCATCAAA GGCTAGCTACAACGA AGACCCAC | 17745 |
| 5844 | UCUGUUUG A UCCUAUUU | 8062 | AAATAGCA GGCTAGCTACAACGA CAAACAGA | 17746 |
| 5846 | UGUUUCAU G CUAUUUAU | 8063 | ATAAATAG GGCTAGCTACAACGA ATCAAACA | 17747 |
| 5849 | UUGAUCCU A UUUAUGCA | 966 | TGCATAAA GGCTAGCTACAACGA AGCATCAA | 17748 |
| 5853 | UGCUAUUU A UGCAAGUU | 969 | AACTTGCA GGCTAGCTACAACGA AAATAGCA | 17749 |
| 5855 | CUAUUUAU G CAAGUUAC | 8064 | CTAACTTG GGCTAGCTACAACGA ATAAATAC | 17750 |
| 5859 | UUAUGCAA G UUAGGCUC | 8065 | CACCCTAA GGCTAGCTACAACGA TTCCATAA | 17751 |
| 5865 | AACUUAGC G UCUAUGUA | 8066 | TACATAGA GGCTAGCTACAACGA CCTAACTT | 17752 |
| 5869 | UAGCCUCU A UCUAUUUA | 973 | TAAATACA GGCTAGCTACAACGA AGACCCTA | 17753 |
| 5871 | CGCUCUAU G UAUUUAGC | 8067 | CCTAAATA GGCTAGCTACAACGA ATAGACCC | 17754 |
| 5873 | GUCUAUCU A UUUACCAU | 974 | ATCCTAAA GGCTAGCTACAACGA ACATAGAC | 17755 |
| 5880 | UAUUUAGC A UCUCUCCA | 8071 | TGCAGACA GGCTAGCTACAACGA CCTAAATA | 17760 |
| 5882 | UUUAGGAU G UCUGCACC | 8072 | CGTGCAGA GGCTAGCTACAACGA ATCCTAAA | 17761 |
| 5886 | GCAUGUCU G CACCUUCU | 8073 | ACAACGTG GGCTAGCTACAACGA ACACATCC | 17762 |
| 5888 | AUGUCUCC A CCUUCUGC | 5335 | GCAGAACG GGCTAGCTACAACGA GCAGACAT | 17763 |
| 5895 | CACCUUCU G CAGCCAGU | 8074 | ACTGGCTC GGCTAGCTACAACGA ACAACCTG | 17764 |
| 5898 | CUUCUGCA G CCACUCAC | 8075 | CTCACTCC GGCTAGCTACAACGA TCCAGAAC | 17765 |
| 5902 | UCCACCCA G UCACAACC | 8076 | CCTTCTGA GGCTAGCTACAACGA TGGCTCCA | 17766 |
| 5909 | ACUCACAA G CUGGACAC | 8077 | CTCTCCAC GGCTAGCTACAACGA TTCTGACT | 17767 |
| 5918 | CUGGACAC G CAACACUG | 8078 | CACTCTTC GGCTAGCTACAACGA CTCTCCAC | 17768 |
| 5921 | GACAGGCA A CAGUCCAU | 8079 | ATCCACTC GGCTAGCTACAACGA TCCCTCTC | 17769 |
| 5924 | ACGCAACA G UCCAUUGC | 8080 | CCAATCCA GGCTAGCTACAACGA TCTTCCCT | 17770 |
| 5928 | AACACUGC A UUCCUGCU | 8081 | AGCACCAA GGCTAGCTACAACGA CCACTGTT | 17771 |
| 5931 | ACUGCAUU G CUGCUUCU | 8082 | ACAAGCAC GGCTAGCTACAACGA AATCCACT | 17772 |
| 5934 | CGAUUGCU G CUUCUUGG | 8083 | CCAACAAG GGCTAGCTACAACGA ACCAATCC | 17773 |
| 5951 | CGACAAGA G UAUCCUUC | 8084 | CAACCATA GGCTAGCTACAACGA TCTTCTCC | 17774 |
| 5953 | ACAAGACU A UGCUUCCU | 990 | AGGAAGCA GGCTAGCTACAACGA ACTCTTCT | 17775 |
| 5955 | AAGAGUAU G CUUCCUUU | 8085 | AAAGCAAG GGCTAGCTACAACGA ATACTCTT | 17776 |
| 5965 | UUCCUUUU A UCCAUCUA | 996 | TACATGGA GGCTAGCTACAACGA AAAACCAA | 17777 |
| 5969 | UUUUAUCC A UGUAAUUU | 5353 | AAATTACA GGCTAGCTACAACGA GCATAAAA | 17778 |
| 5971 | UUAUCCAU G UAAUUUAA | 8086 | TTAAATTA GGCTAGCTACAACGA ATCCATAA | 17779 |
| 5974 | UCCAUCUA A UUUAACUG | 8087 | CAGTTAAA GGCTAGCTACAACGA TACATGCA | 17780 |
| 5979 | CUAAUUUA A CUGUACAA | 8088 | TTCTACAG GGCTAGCTACAACGA TAAATTAC | 17781 |
| 5982 | AUUUAACU G UACAACCU | 8089 | ACCTTCTA GGCTAGCTACAACGA ACTTAAAT | 17782 |
| 5987 | ACUGUAGA A CCUGAGCU | 8090 | AGCTCAGG GGCTAGCTACAACGA TCTACAGT | 17783 |
| 5993 | GAACCUGA G CUCUAAGU | 8091 | ACTTAGAG GGCTAGCTACAACGA TCAGGTTC | 17784 |
| 6000 | AGCUCUAA G UAACCGAA | 8092 | TTCGGTTA GGCTAGCTACAACGA TTAGAGCT | 17785 |
| 6003 | UCUAAGUA A CCCAAGAA | 8093 | TTCTTCGG GGCTAGCTACAACGA TACTTAGA | 17786 |
| 6011 | ACCGAAGA A UGUAUGCC | 8094 | GGCATACA GGCTAGCTACAACGA TCTTCGGT | 17787 |
| 6013 | CGAAGAAU G UAUGCCUC | 8095 | GACGCATA GGCTAGCTACAACGA ATTCTTCC | 17788 |
| 6015 | AAGAAUGU A UGCCUCUG | 1006 | CAGAGGCA GGCTAGCTACAACGA ACATTCTT | 17789 |
| 6017 | CAAUGUAU G CCUCUCUU | 8096 | AACAGAGG GGCTAGCTACAACGA ATACATTC | 17790 |
| 6023 | AUGGCUCH G UUCUUAUG | 8097 | CATAAGAA GGCTAGCTACAACGA AGAGGCAT | 17791 |
| 6029 | CUGUUCUU A UGUGCCAC | 1011 | GTGCCACA GGCTAGCTACAACGA AAGAACAG | 17792 |
| 6031 | GUUCUUAU G UGCCACAU | 8098 | ATGTCGCA GGCTAGCTACAACGA ATAAGAAC | 17793 |
| 6033 | UCUUAUGU G CCACAUCC | 8099 | CCATGTGC GGCTAGCTACAACGA ACATAACA | 17794 |
| 6036 | UAUGUCCC A CAUCCUUG | 5365 | CAAGCATC GGCTAGCTACAACGA GGCACATA | 17795 |
| 6038 | UGUGCCAC A UCCUUCUU | 5366 | AACAAGCA GGCTAGCTACAACGA GTGCCACA | 17796 |
| 6044 | ACAUCCUU G UUUAAAGG | 8100 | CCTTTAAA GGCTAGCTACAACGA AAGCATGT | 17797 |
| 6052 | CUUUAAAC G CUCUCUGU | 8101 | ACAGAGAG GGCTAGCTACAACGA CTTTAAAC | 17798 |
| 6059 | CGCUCUCU G UAUGAAGA | 8102 | TCTTCATA GGCTAGCTACAACGA AGACACCC | 17799 |
| 6061 | CUCUCUGU A UCAACACA | 1019 | TCTCTTCA GGCTAGCTACAACGA ACACAGAG | 17800 |
| 6069 | AUGAACAG A UCGGACCC | 8103 | CCGTCCCA GGCTAGCTACAACGA CTCTTCAT | 17801 |
| 6074 | CAGAUGGC A CCCUGAUG | 8104 | CATCACGG GGCTAGCTACAACGA CCCATCTC | 17802 |
| 6077 | AUCCGACC G UGAUGAGC | 8105 | CCTGATCA GGCTAGCTACAACGA GCTCCCAT | 17803 |
| 6080 | CCACCGUC A UCAGCACA | 5373 | TGTCCTGA GGCTAGCTACAACGA CACGGTCC | 17804 |
| 6084 | CCUGAUGA G CACAUUCC | 8106 | GCAATGTG GGCTAGCTACAACGA TCATGACC | 17805 |
| 6086 | UGAUGACC A CAUUCCCU | 5375 | ACGGAATC GGCTAGCTACAACGA CCTGATCA | 17806 |
| 6088 | AUCAGCAC A UUCCCUAC | 5376 | CTACCCAA GGCTAGCTACAACGA GTCCTGAT | 17807 |
| 6096 | AUUCCCUA G UGACCCUA | 8107 | TAGCCTCA GGCTAGCTACAACGA TAGGGAAT | 17808 |
| 6100 | CCUACUCA G CCUACUGG | 8108 | CCACTACC GGCTAGCTACAACGA TCACTACG | 17809 |
| 6104 | CUCAGCCU A CUGGCUCC | 1025 | GGAGCCAG GGCTAGCTACAACGA ACGCTCAC | 17810 |
| 6108 | GCCUACUG G CUCCUCGC | 8109 | CCCACGAC GGCTAGCTACAACGA CAGTACGC | 17811 |
| 6115 | GGCUCCUG G CACCCGCU | 8110 | ACCCGCTC GGCTAGCTACAACGA CACCAGCC | 17812 |
| 6118 | UCCUGCCA G CCGCUUUU | 8111 | AAAAGCCC GGCTAGCTACAACGA TGCCAGCA | 17813 |
| 6121 | UGGCACCC G CUUUUCUC | 8112 | CACAAAAG GGCTAGCTACAACGA CCCTCCCA | 17814 |
| 6127 | CCCCUUUU G UCCAACAC | 8113 | CTCTTCCA GGCTAGCTACAACGA AAAACCCC | 17815 |
| 6134 | UGUCCAAC A CUCACUAC | 8114 | CTACTCAC GGCTAGCTACAACGA CTTCCACA | 17816 |
| 6138 | CAACACUC A CUACCCAC | 5389 | CTCCCTAC GGCTAGCTACAACGA CAGTCTTC | 17817 |
| 6142 | ACUCACUA G CCACAACA | 8115 | TCTTCTCC GGCTAGCTACAACGA TACTCACT | 17818 |
| 6156 | ACACACCA G UCCGACAC | 8116 | CTCTCCCA GGCTAGCTACAACGA TCCTCTCT | 17819 |
| 6161 | CCACUCCC A CACUCCUC | 8117 | CACCACTC GGCTAGCTACAACGA CCCACTCC | 17820 |
| 6164 | CUCGCACA G UCCUCUCC | 8118 | GCACACCA GGCTAGCTACAACGA TCTCCCAC | 17821 |
| 6173 | UCCUCUCC A CCAACAUC | 5398 | CATCTTCC GGCTAGCTACAACGA CGACACCA | 17822 |
| 6179 | CCACCAAC A UCUAAAUC | 8119 | CATTTACA GGCTAGCTACAACGA CTTCCTCC | 17823 |
| 6185 | ACAUCUAA A UCCAAACA | 8120 | TCTTTCCA GGCTAGCTACAACGA TTACATCT | 17824 |
| 6191 | AAAUCCAA A CAAAACCA | 8121 | TCCTTTTC GGCTAGCTACAACGA TTCCATTT | 17825 |
| 6197 | AAACAAAA G CACCCUAC | 8122 | CTACCCTC GGCTAGCTACAACGA TTTTCTTT | 17826 |
| 6201 | AAAACCAC G CUACACCC | 8123 | CCCTCTAC GGCTAGCTACAACGA CTCCTTTT | 17827 |
| 6207 | ACCCUACA G CCACAACA | 8124 | TCTTCTCC GGCTAGCTACAACGA TCTACCCT | 17828 |
| 6220 | AACACACC A CAAAUCUU | 8125 | AACATTTC GGCTAGCTACAACGA CCTCTCTT | 17829 |
| 6224 | CACCACAA A UCUUUCUU | 8126 | AACAAACA GGCTAGCTACAACGA TTCTCCTC | 17830 |
| 6230 | AAAUCUUU G UUCUUCCU | 8127 | ACCAACAA GGCTAGCTACAACGA AAACATTT | 17831 |
| 6233 | UCUUUGUU G UUCCUCUU | 8128 | AAGAGGAA GGCTAGCTACAACGA AACAAAGA | 17832 |
| 6246 | UCUUCUUU A CACAUACG | 1050 | CGTATGTG GGCTAGCTACAACGA AAAGAAGA | 17833 |
| 6248 | UUCUUUAC A CAUACCCA | 5415 | TGCGTATG GGCTAGCTACAACGA GTAAAGAA | 17834 |
| 6250 | CUUUACAC A UACCCAAA | 5416 | TTTGCGTA GGCTAGCTACAACGA GTGTAAAG | 17835 |
| 6252 | UUACACAU A CGCAAACC | 1051 | GGTTTCCG GGCTAGCTACAACGA ATGTGTAA | 17836 |
| 6254 | ACACAUAC G CAAACCAC | 8129 | GTGGTTTG GGCTAGCTACAACGA GTATGTGT | 17837 |
| 6258 | AUACCCAA A CCACCUCU | 8130 | ACAGGTGG GGCTAGCTACAACGA TTGCGTAT | 17838 |
| 6261 | CUCAAACC A CCUGUGAC | 5419 | GTCACAGG GGCTAGCTACAACGA GGTTTGCG | 17839 |
| 6265 | AACCACCU G UGACAGCU | 8131 | AGCTGTCA GGCTAGCTACAACGA AGGTGGTT | 17840 |
| 6268 | CACCUGUG A CAGCUGGC | 8132 | GCCAGCTG GGCTAGCTACAACGA CACAGGTG | 17841 |
| 6271 | CUGUGACA G CUGGCAAU | 8133 | ATTGCCAG GGCTAGCTACAACGA TGTCACAG | 17842 |
| 6275 | GACAUCUG G CAAUUUUA | 8134 | TAAAATTG GGCTAGCTACAACGA CAGCTGTC | 17843 |
| 6278 | AUCUGGCA A UUUUAUAA | 8135 | TTATAAAA GGCTAGCTACAACGA TGCCAGCT | 17844 |
| 6283 | UCAAUUUU A UAAAUCAU | 1055 | CTGATTTA GGCTAGCTACAACGA AAAATTGC | 17845 |
| 6287 | UUUUAUAA A UCAGUUAA | 8136 | TTACCTGA GGCTAGCTACAACGA TTATAAAA | 17846 |
| 6292 | UAAAUCAG G UAACUUGA | 8137 | TCCAGTTA GGCTAGCTACAACGA CTGATTTA | 17847 |
| 6295 | AUCAGGUA A CUGGAAUG | 8138 | CCTTCCAG GGCTAGCTACAACGA TACCTGAT | 17848 |
| 6306 | UGAAGGAG G UUAAACUC | 8139 | GAGTTTAA GGCTAGCTACAACGA CTCCTTCC | 17849 |
| 6311 | GAGGUUAA A CUCAGAAA | 8140 | TTTCTGAG GGCTAGCTACAACGA TTAACCTC | 17850 |
| 6327 | AAAAGAAG A CCUCAUUC | 8141 | GACTGAGG GGCTAGCTACAACGA CTTCTTTT | 17851 |
| 6333 | AGACCUCA G UCAAUUCU | 8142 | AGAATTGA GGCTAGCTACAACGA TGAGGTCT | 17852 |
| 6337 | CUCAGUCA A UUCUCUAC | 8143 | GTAGAGAA GGCTAGCTACAACGA TGACTGAG | 17853 |
| 6344 | AAUUCUCU A CUUUUUUU | 1067 | AAAAAAAG GGCTAGCTACAACGA AGAGAATT | 17854 |
| 6366 | UUUUCCAA A UCAUAUAA | 8144 | TTATCTGA GGCTAGCTACAACGA TTGGAAAA | 17855 |
| 6371 | CAAAUCAG A UAAUAGCC | 8145 | GGCTATTA GGCTAGCTACAACGA CTGATTTG | 17856 |
| 6374 | AUCAGAUA A UAGCCCAG | 8146 | CTGGGCTA GGCTAGCTACAACGA TATCTGAT | 17857 |
| 6377 | AGAUAAUA G CCCAGCAA | 8147 | TTGCTGGG GGCTAGCTACAACGA TATTATCT | 17858 |
| 6382 | AUAUCCCA G CAAAUAGU | 8148 | ACTATTTG GGCTAGCTACAACGA TGGGCTAT | 17859 |
| 6386 | CCCAGCAA A UAGUGAUA | 8149 | TATCACTA GGCTAGCTACAACGA TTGCTGGG | 17860 |
| 6389 | AGCAAAUA G UGAUAACA | 8150 | TGTTATCA GGCTAGCTACAACGA TATTTGCT | 17861 |
| 6392 | AAAUAUUU A UAACAAAU | 8151 | ATTTGTTA GGCTAGCTACAACGA CACTATTT | 17862 |
| 6395 | UAUUUAUA A CAAAUAAA | 8152 | TTTATTTG GGCTAGCTACAACGA TATCACTA | 17863 |
| 6399 | UAUAACAA A UAAAACCU | 8153 | AGGTTTTA GGCTAGCTACAACGA TTGTTATC | 17864 |
| 6404 | CAAAUAAA A CCUUAUCU | 8154 | AGCTAAGG GGCTAGCTACAACGA TTTATTTG | 17865 |
| 6410 | AAACCUUA G CUUUUCAU | 8155 | ATGAACAG GGCTAGCTACAACGA TAAGGTTT | 17866 |
| 6413 | CCUUAUCU G UUCAUUUC | 8156 | GACATGAA GGCTAGCTACAACGA AUCTAAGG | 17867 |
| 6417 | AUCUUUUC A UUUCUUUA | 5447 | TCAAGACA GGCTAGCTACAACGA GAACAGCT | 17868 |
| 6419 | CUUUUCAU G UCUUUAUU | 8157 | AATCAAGA GGCTAGCTACAACGA ATGAACAG | 17869 |
| 6425 | AUGUCUUG A UUUCAAUA | 8158 | TATTGAAA GGCTAGCTACAACGA CAAGACAT | 17870 |
| 6431 | UGAUUUCA A UAAUUAAU | 8159 | ATTAATTA GGCTAGCTACAACGA TGAAATCA | 17871 |
| 6434 | UUUCAAUA A UUAAUUCU | 8160 | AGAATTAA GGCTAGCTACAACGA TATTGAAA | 17872 |
| 6438 | AAUAAUUA A UUCUUAAU | 8161 | ATTAAGAA GGCTAGCTACAACGA TAATTATT | 17873 |
| 6445 | AAUUCUUA A UCAUUAAU | 8162 | CTTAATGA GGCTAGCTACAACGA TAAGAATT | 17874 |
| 6448 | UCUUAAUC A UUAAUAGA | 5451 | TCTCTTAA GGCTAGCTACAACGA GATTAAGA | 17875 |
| 6456 | AUUAAUAU A CCAUAAUA | 8163 | TATTATGG GGCTAGCTACAACGA CTCTTAAT | 17876 |
| 6459 | AAUAUACC A UAAUAAAU | 5453 | ATTTATTA GGCTAGCTACAACGA GGTCTCTT | 17877 |
| 6462 | AUACCAUA A UAAAUACU | 8164 | AGTATTTA GGCTAGCTACAACGA TATGGTCT | 17878 |
| 6466 | CAUAAUAA A UACUCCUU | 8165 | AAGGAGTA GGCTAGCTACAACGA TTATTATG | 17879 |
| 6468 | UAAUAAAU A CUCCUUUU | 1111 | AAAAGGAG GGCTAGCTACAACGA ATTTATTA | 17880 |
| 6487 | AGACAAAA G CAAAACCA | 8166 | TGGTTTTG GGCTAGCTACAACGA TTTTCTCT | 17881 |
| 6492 | AAAGCAAA A CCAUUAGA | 8167 | TCTAATGG GGCTAGCTACAACGA TTTGCTTT | 17882 |
| 6495 | GCAAAACC A UUAGAAUU | 5460 | AATTCTAA GGCTAGCTACAACGA GGTTTTGC | 17883 |
| 6501 | CCAUUAGA A UUGUUACU | 8168 | AGTAACAA GGCTAGCTACAACGA TCTAATGG | 17884 |
| 6504 | UUAGAAUU G UUACUCAG | 8169 | CTGAGTAA GGCTAGCTACAACGA AATTCTAA | 17885 |
| 6507 | GAAUUGUU A CUCAGCUC | 1121 | GAGCTGAG GGCTAGCTACAACGA AACAATTC | 17886 |
| 6512 | GUUACUCA G CUCCUUCA | 8170 | TGAAGGAG GGCTAGCTACAACGA TCAGTAAC | 17887 |
| 6522 | UCCUUCAA A CUCAGGUU | 8171 | AACCTGAG GGCTAGCTACAACGA TTGAAGGA | 17888 |
| 6528 | AAACUCAG G UUUCUAGC | 8172 | GCTACAAA GGCTAGCTACAACGA CTGAGTTT | 17889 |
| 6532 | UCAGGUUU G UAGCAUAC | 8173 | GTATGCTA GGCTAGCTACAACGA AAACCTGA | 17890 |
| 6535 | GGUUUGUA G CACACAUG | 8174 | CATGTATG GGCTAGCTACAACGA TACAAACC | 17891 |
| 6537 | UUUGUAGC A UACAUGAC | 5469 | CTCATGTA GGCTAGCTACAACGA GCTACAAA | 17892 |
| 6539 | UGUAGCAU A CAUGAGUC | 1130 | GACTCATG GGCTAGCTACAACGA ATGCTACA | 17893 |
| 6541 | UAGCAUAC A UGAGUCCA | 5470 | TGGACTCA GGCTAGCTACAACGA GTATGCTA | 17894 |
| 6545 | AUACAUGA G UCCAUCCA | 8175 | TGGATGGA GGCTAGCTACAACGA TCATGTAT | 17895 |
| 6549 | AUGAGUCC A UCCAUCAG | 5472 | CTGATGGA GGCTAGCTACAACGA GGACTCAT | 17896 |
| 6553 | GUCCAUCC A UCAGUCAA | 5474 | TTGACTGA GGCTAGCTACAACGA GGATGGAC | 17897 |
| 6557 | AUCCAUCA G UCAAAGAA | 8176 | TTCTTTGA GGCTAGCTACAACGA TGATGGAT | 17898 |
| 6565 | GUCAAAGA A UGGUUCCA | 8177 | TGGAACCA GGCTAGCTACAACGA TCTTTGAC | 17899 |
| 6568 | AAAGAAUG G UUCCAUCU | 8178 | AGATGGAA GGCTAGCTACAACGA CATTCTTT | 17900 |
| 6573 | AUGGUUGC A UCUGGAGU | 5478 | ACTCCAGA GGCTAGCTACAACGA GGAACCAT | 17901 |
| 6580 | CAUCUGGA G UCUUAAUG | 8179 | CATTAAGA GGCTAGCTACAACGA TCCAGATG | 17902 |
| 6586 | GAGUCUUA A UGUAGAAA | 8180 | TTTCTACA GGCTAGCTACAACGA TAAGACTC | 17903 |
| 6588 | GUCUUAAU G UAGAAAGA | 8181 | TCTTTCTA GGCTAGCTACAACGA ATTAAGAC | 17904 |
| 6600 | AAAGAAAA A UGGAGACU | 8182 | AGTCTCCA GGCTAGCTACAACGA TTTTCTTT | 17905 |
| 6606 | AAAUGGAG A CUUGUAAU | 8183 | ATTACAAG GGCTAGCTACAACGA CTCCATTT | 17906 |
| 6610 | GGAGACUU G UAAUAAUG | 8184 | CATTATTA GGCTAGCTACAACGA AAGTCTCC | 17907 |
| 6613 | GACUUGUA A UAAUGAGC | 8185 | GCTCATTA GGCTAGCTACAACGA TACAAGTC | 17908 |
| 6616 | UUGUAAUA A UGAGCUAG | 8186 | CTAGCTCA GGCTAGCTACAACGA TATTACAA | 17909 |
| 6620 | AAUAAUGA G CUAGUCAC | 8187 | GTAACTAG GGCTAGCTACAACGA TCATTATT | 17910 |
| 6624 | AUGAGCUA G UUACAAAG | 8188 | CTTTGTAA GGCTAGCTACAACGA TAGCTCAT | 17911 |
| 6627 | AGCUAGUU A CAAAGUGC | 1147 | GCACTTTG GGCTAGCTACAACGA AACTAGCT | 17912 |
| 6632 | GUUACAAA G UGCUUGUU | 8189 | AACAAGCA GGCTAGCTACAACGA TTTGTAAC | 17913 |
| 6634 | UACAAAGU G CUUGUUCA | 8190 | TGAACAAG GGCTAGCTACAACGA ACTTTGTA | 17914 |
| 6638 | AAGUGCUU G UUCAUUAA | 8191 | TTAATGAA GGCTAGCTACAACGA AAGCACTT | 17915 |
| 6642 | GCUUGUUC A UUAAAAUA | 5485 | TATTTTAA GGCTAGCTACAACGA GAACAAGC | 17916 |
| 6648 | UCAUUAAA A UAGCACUG | 8192 | CAGTGCTA GGCTAGCTACAACGA TTTAATGA | 17917 |
| 6651 | UUAAAAUA G CACUGAAA | 8193 | TTTCAGTG GGCTAGCTACAACGA TATTTTAA | 17918 |
| 6653 | AAAAUAGC A CUGAAAAU | 5486 | ATTTTCAG GGCTAGCTACAACGA GCTATTTT | 17919 |
| 6660 | CACUGAAA A UUGAAACA | 8194 | TGTTTCAA GGCTAGCTACAACGA TTTCAGTG | 17920 |
| 6666 | AAAUUGAA A CAUGAAUU | 8195 | AATTCATG GGCTAGCTACAACGA TTCAATTT | 17921 |
| 6668 | AUUGAAAC A UGAAUUAA | 5488 | TTAATTCA GGCTAGCTACAACGA GTTTCAAT | 17922 |
| 6672 | AAACAUGA A UUAACUGA | 8196 | TCAGTTAA GGCTAGCTACAACGA TCATGTTT | 17923 |
| 6676 | AUGAAUUA A CUGAUAAU | 8197 | ATTATCAG GGCTAGCTACAACGA TAATTCAT | 17924 |
| 6680 | AUUAACUG A UAAUAUUC | 8198 | GAATATTA GGCTAGCTACAACGA CAGTTAAT | 17925 |
| 6683 | AACUGAUA A UAUUCCAA | 8199 | TTGGAATA GGCTAGCTACAACGA TATCAGTT | 17926 |
| 6685 | CUGAUAAU A UUCCAAUC | 1158 | GATTGGAA GGCTAGCTACAACGA ATTATCAG | 17927 |
| 6691 | AUAUUCCA A UCAUUUGC | 8200 | GCAAATGA GGCTAGCTACAACGA TGGAATAT | 17928 |
| 6694 | UUCCAAUC A UUUGCCAU | 5492 | ATGGCAAA GGCTAGCTACAACGA GATTGGAA | 17929 |
| 6698 | AAUCAUUU G CCAUUUAU | 8201 | ATAAATGG GGCTAGCTACAACGA AAATGATT | 17930 |
| 6701 | CAUUUGCC A UUUAUGAC | 5494 | GTCATAAA GGCTAGCTACAACGA GGCAAATG | 17931 |
| 6705 | UUCCAUUU A UGACAAAA | 1166 | TTTTGTCA GGCTAGCTACAACGA AAATGGCA | 17932 |
| 6708 | CAUUUAUG A CAAAAAUG | 8202 | CATTTTTG GGCTAGCTACAACGA CATAAATG | 17933 |
| 6714 | UGACAAAA A UGGUUGGC | 8203 | GCCAACCA GGCTAGCTACAACGA TTTTGTCA | 17934 |
| 6717 | CAAAAAUG G UUGGCACU | 8204 | AGTGCCAA GGCTAGCTACAACGA CATTTTTG | 17935 |
| 6721 | AAUGGUUG G CACUAACA | 8205 | TGTTAGTG GGCTAGCTACAACGA CAACCATT | 17936 |
| 6723 | UGGUUGGC A CUAACAAA | 5496 | TTTGTTAG GGCTAGCTACAACGA CCCAACCA | 17937 |
| 6727 | UGGCACUA A CAAAGAAC | 8206 | GTTCTTTG GGCTAGCTACAACGA TAGTGCCA | 17938 |
| 6734 | AACAAAGA A CGAGCACU | 8207 | AGTGCTCG GGCTAGCTACAACGA TCTTTGTT | 17939 |
| 6738 | AAGAACGA G CACUUCCU | 8208 | AGCAAGTG GGCTAGCTACAACGA TCGTTCTT | 17940 |
| 6740 | GAACGAGC A CUUCCUUU | 5499 | AAAGGAAG GGCTAGCTACAACGA GCTCGTTC | 17941 |
| 6753 | CUUUCAGA G UUUCUGAG | 8209 | CTCAGAAA GGCTAGCTACAACGA TCTGAAAG | 17942 |
| 6762 | UUUCUGAG A UAAUGUAC | 8210 | GTACATTA GGCTAGCTACAACGA CTCAGAAA | 17943 |
| 6765 | CUGAGAUA A UGUACGUG | 8211 | CACGTACA GGCTAGCTACAACGA TATCTCAG | 17944 |
| 6767 | GAGAUAAU G UACGUGGA | 8212 | TCCACGTA GGCTAGCTACAACGA ATTATCTC | 17945 |
| 6769 | GAUAAUGU A CGUGGAAC | 1178 | GTTCCACG GGCTAGCTACAACGA ACATTATC | 17946 |
| 6771 | UAAUGUAC G UGGAACAG | 8213 | CTGTTCCA GGCTAGCTACAACGA GTACATTA | 17947 |
| 6776 | UACGUGGA A CAGUCUGG | 8214 | CCAGACTG GGCTAGCTACAACGA TCCACGTA | 17948 |
| 6779 | GUGGAACA G UCUGGGUG | 8215 | CACCCAGA GGCTAGCTACAACGA TGTTCCAC | 17949 |
| 6785 | CAGUCUGG G UGGAAUGG | 8216 | CCATTCCA GGCTAGCTACAACGA CCAGACTG | 17950 |
| 6790 | UGGGUGGA A UCGGGCUG | 8217 | CAGCCCCA GGCTAGCTACAACGA TCCACCCA | 17951 |
| 6795 | GGAAUGGG G CUGAAACC | 8218 | GGTTTCAG GGCTAGCTACAACGA CCCATTCC | 17952 |
| 6801 | GGCCUCAA A CCAUGUGC | 8219 | GCACATCC GGCTAGCTACAACGA TTCAGCCC | 17953 |
| 6804 | CUGAAACC A UGUGCAAG | 5509 | CTTGCACA GGCTAGCTACAACGA GGTTTCAG | 17954 |
| 6806 | GAAACCAU G UGCAACUC | 8220 | GACTTGCA GGCTAGCTACAACGA ATGCTTTC | 17955 |
| 6808 | AACCAUGU G CAAGUCUG | 8221 | CAGACTTC GGCTAGCTACAACGA ACATGCTT | 17956 |
| 6812 | AUCUCCAA G UCUGUGUC | 8222 | CACACAGA GGCTAGCTACAACGA TTGCACAT | 17957 |
| 6816 | CCAACUCU G UGUCUUCU | 8223 | ACAAGACA GGCTAGCTACAACGA AGACTTCC | 17958 |
| 6818 | AACUCUCU G UCUUGUCA | 8224 | TCACAAGA GGCTAGCTACAACGA ACACACTT | 17959 |
| 6823 | UCUCUCUU G UCACUCCA | 8225 | TCGACTGA GGCTAGCTACAACGA AAGACACA | 17960 |
| 6827 | UCUGGUCA G UCCAAGAA | 8226 | TTCTTCCA GGCTAGCTACAACGA TGACAAGA | 17961 |
| 6836 | UCCAAGAA G UGACACCG | 8227 | CGGTCTCA GGCTAGCTACAACGA TTCTTGGA | 17962 |
| 6839 | AAGAAGUG A CACCCAGA | 8228 | TCTCGGTG GGCTAGCTACAACGA CACTTCTT | 17963 |
| 6841 | GAAGUCAC A CCGAGAUC | 5516 | CATCTCCG GGCTAGCTACAACGA CTCACTTC | 17964 |
| 6847 | ACACCGAC A UGUUAAUU | 8229 | AATTAACA GGCTAGCTACAACGA CTCGGTCT | 17965 |
| 6849 | ACCGAGAU G UUAAUUUU | 8230 | AAAATTAA GGCTAGCTACAACGA ATCTCGGT | 17966 |
| 6853 | AGAUGUUA A UUUUAGGG | 8231 | CCCTAAAA GGCTAGCTACAACGA TAACATCT | 17967 |
| 6862 | UUUUAGGG A CCCGUCCC | 8232 | GGCACGGG GGCTAGCTACAACGA CCCTAAAA | 17968 |
| 6866 | AGGGACCC G UCCCUUGU | 8233 | ACAAGGCA GGCTAGCTACAACGA CCGTCCCT | 17969 |
| 6868 | GCACCCGU G CCUUGUUU | 8234 | AAACAAGG GGCTAGCTACAACGA ACGGCTCC | 17970 |
| 6873 | CGUGCCUU G UUUCCUAG | 8235 | CTACCAAA GGCTAGCTACAACGA AAGGCACG | 17971 |
| 6881 | GUUUCCUA G CCCACAAG | 8236 | CTTGTCGG GGCTAGCTACAACGA TAGGAAAC | 17972 |
| 6885 | CCUAGCCC A CAAGAAUC | 5526 | CATTCTTG GGCTAGCTACAACGA GGGCTACG | 17973 |
| 6891 | CCACAAGA A UGCAAACA | 8237 | TGTTTGCA GGCTAGCTACAACGA TCTTGTGG | 17974 |
| 6893 | ACAAGAAU G CAAACAUC | 8238 | GATGTTTG GGCTAGCTACAACGA ATTCTTGT | 17975 |
| 6897 | GAAUCCAA A CAUCAAAC | 8239 | GTTTGATG GGCTAGCTACAACGA TTCCATTC | 17976 |
| 6899 | AUGCAAAC A UCAAACAC | 5529 | CTCTTTGA GGCTAGCTACAACGA GTTTGCAT | 17977 |
| 6904 | AACAUCAA A CACAUACU | 8240 | AGTATCTG GGCTAGCTACAACGA TTCATGTT | 17978 |
| 6908 | UCAAACAG A UACUCGCU | 8241 | AGCGAGTA GGCTAGCTACAACGA CTGTTTGA | 17979 |
| 6910 | AAACAGAU A CUCGCUAG | 1197 | CTAGCGAG GGCTAGCTACAACGA ATCTGTTT | 17980 |
| 6914 | AGAUACUC G CUACCCUC | 8242 | GAGGCTAG GGCTAGCTACAACGA GAGTATCT | 17981 |
| 6918 | ACUCGCUA G CCUCAUUU | 8243 | AAATGAGG GGCTAGCTACAACGA TAGCGAGT | 17982 |
| 6923 | CUAGCCUC A UUUAAAUU | 5536 | AATTTAAA GGCTAGCTACAACGA GAGGCTAG | 17983 |
| 6929 | UCAUUUAA A UUGAUUAA | 8244 | TTAATCAA GGCTAGCTACAACGA TTAAATGA | 17984 |
| 6933 | UUAAAUUG A UUAAAGGA | 8245 | TCCTTTAA GGCTAGCTACAACGA CAATTTAA | 17985 |
| 6945 | AAGGAGGA G UCCAUCUU | 8246 | AAGATGCA GGCTAGCTACAACGA TCCTCCTT | 17986 |
| 6947 | GGAGGAGU G CAUCUUUC | 8247 | CAAAGATC GGCTAGCTACAACGA ACTCCTCC | 17987 |
| 6949 | AGGAGUGC A UCUUUGGC | 5537 | CCCAAAGA GGCTAGCTACAACGA GCACTCCT | 17988 |
| 6956 | CAUCUUUG G CCGACAGU | 8248 | ACTCTCGC GGCTAGCTACAACGA CAAACATG | 17989 |
| 6960 | UUUGGCCG A CAGUGGUG | 8249 | CACCACTC GGCTAGCTACAACGA CGGCCAAA | 17990 |
| 6963 | GGCCCACA G UGGUGUAA | 8250 | TTACACCA GGCTAGCTACAACGA TGTCGCCC | 17991 |
| 6966 | CGACAGUG G UGUAACUC | 8251 | CAGTTACA GGCTAGCTACAACGA CACTGTCG | 17992 |
| 6968 | ACAGUGCU G UAACUGUC | 8252 | CACAGTTA GGCTAGCTACAACGA ACCACTGT | 17993 |
| 6971 | CUCGUGUA A CUCUGUCU | 8253 | ACACACAC GGCTAGCTACAACGA TACACCAC | 17994 |
| 6974 | CUCUAACU G UCUCUGUG | 8254 | CACACACA GGCTAGCTACAACGA AGTTACAC | 17995 |
| 6976 | CUAACUCU G UGUCUGUG | 8255 | CACACACA GGCTAGCTACAACGA ACAGTTAC | 17996 |
| 6978 | AACUGUGU G UGUGUGUG | 8256 | CACACACA GGCTAGCTACAACGA ACACACTT | 17997 |
| 6980 | CUGUGUGU G UGUCUGUG | 8257 | CACACACA GGCTAGCTACAACGA ACACACAG | 17998 |
| 6982 | GUCUGUGU G UGUGUGUG | 8258 | CACACACA GGCTAGCTACAACGA ACACACAC | 17999 |
| 6984 | GUGUGUGU G UGUGUGUG | 8258 | CACACACA GGCTAGCTACAACGA ACACACAC | 17999 |
| 6986 | GUCUGUGU G UGUGUGUG | 8258 | CACACACA GGCTAGCTACAACGA ACACACAC | 17999 |
| 6988 | GUGUCUGU G UGUGUGUG | 8258 | CACACACA GGCTAGCTACAACGA ACACACAC | 17999 |
| 6990 | GUGUGUGU G UGUGUGUG | 8258 | CACACACA GGCTAGCTACAACGA ACACACAC | 17999 |
| 6992 | GUGUCUCU G UGUCUCUC | 8258 | CACACACA GGCTAGCTACAACGA ACACACAC | 17999 |
| 6994 | CUCUGUGU G UGUGUGUG | 8258 | CACACACA GGCTAGCTACAACGA ACACACAC | 17999 |
| 6996 | GUCUCUGU G UCUCUGUG | 8258 | CACACACA GGCTAGCTACAACGA ACACACAC | 17999 |
| 6998 | CUCUGUGU G UGUGUGUG | 8258 | CACACACA GGCTAGCTACAACGA ACACACAC | 17999 |
| 7000 | GUGUGUGU G UGUGUGUG | 8258 | CACACACA GGCTAGCTACAACGA ACACACAC | 17999 |
| 7002 | GUGUGUGU G UGUCUGUG | 8258 | CACACACA GGCTAGCTACAACGA ACACACAC | 17999 |
| 7004 | GUCUCUGU G UGUGUCUG | 8258 | CACACACA GGCTAGCTACAACGA ACACACAC | 17999 |
| 7006 | CUGUGUGU G UGUGUCUG | 8258 | CACACACA GGCTAGCTACAACGA ACACACAC | 17999 |
| 7008 | CUCUCUCU G UCUCUCCC | 8259 | CCCACACA GGCTAGCTACAACGA ACACACAC | 18000 |
| 7010 | CUCUCUCU G UCUCGCUC | 8260 | CACCCACA GGCTAGCTACAACGA ACACACAC | 18001 |
| 7012 | GUCUGUCU G UCCCUCUC | 8261 | CACACCCA GGCTAGCTACAACGA ACACACAC | 18002 |
| 7016 | GUGUCUCG G UCUCGCUC | 8262 | CACCCACA GGCTAGCTACAACGA CCACACAC | 18003 |
| 7018 | CUCUCCCU G UGGCUCUA | 8263 | TACACCCA GGCTAGCTACAACGA ACCCACAC | 18004 |
| 7022 | GGCUGUGG G UGUAUGUC | 8264 | CACATACA GGCTAGCTACAACGA CCACACCC | 18005 |
| 7024 | CUCUGCCU G UAUGUGUG | 8265 | CACACATA GGCTAGCTACAACGA ACCCACAC | 18006 |
| 7026 | GUGGGUGU A UCUCUCUU | 1211 | AACACACA GGCTAGCTACAACGA ACACCCAC | 18007 |
| 7028 | CCCUCUAU G UGUCUUUU | 8266 | AAAACACA GGCTAGCTACAACGA ATACACCC | 18008 |
| 7030 | CUGUAUCU G UGUUUUCU | 8267 | ACAAAACA GGCTAGCTACAACGA ACATACAC | 18009 |
| 7032 | CUAUCUGU G UUUUCUCC | 8268 | CCACAAAA GGCTAGCTACAACGA ACACATAC | 18010 |
| 7037 | UCUCUUUU G UCCAUAAC | 8269 | GTTATGCA GGCTAGCTACAACGA AAAACACA | 18011 |
| 7039 | UGUUUUCU G CAUAACUA | 8270 | TACTTATC GGCTAGCTACAACGA ACAAAACA | 18012 |
| 7041 | UUUUCUCC A UAACUAUU | 5542 | AATACTTA GGCTAGCTACAACGA GCACAAAA | 18013 |
| 7044 | UGUGCAUA A CUAUUUAA | 8271 | TTAAATAC GGCTAGCTACAACGA TATGCACA | 18014 |
| 7047 | GCAUAACU A UUUAACCA | 1216 | TCCTTAAA GGCTAGCTACAACGA ACTTATCC | 18015 |
| 7057 | UUAAGCAA A CUGGAAUU | 8272 | AATTCCAG GGCTAGCTACAACGA TTCCTTAA | 18016 |
| 7063 | AAACUGGA A UUUUAAAG | 8273 | CTTTAAAA GGCTAGCTACAACGA TCCAGTTT | 18017 |
| 7071 | AUUUUAAA G UUACUUUU | 8274 | AAAAGTAA GGCTAGCTACAACGA TTTAAAAT | 18018 |
| 7074 | UUAAAGUU A CUUUUAUA | 1225 | TATAAAAG GGCTAGCTACAACGA AACTTTAA | 18019 |
| 7080 | UUACUUUU A UACAAACC | 1229 | GGTTTGTA GGCTAGCTACAACGA AAAAGTAA | 18020 |
| 7082 | ACUUUUAU A CAAACCAA | 1230 | TTGGTTTG GGCTAGCTACAACGA ATAAAAGT | 18021 |
| 7086 | UUAUACAA A CCAACAAU | 8275 | ATTCTTGG GGCTAGCTACAACGA TTGTATAA | 18022 |
| 7093 | AACCAAGA A UAUAUGCU | 8276 | AGCATATA GGCTAGCTACAACGA TCTTGCTT | 18023 |
| 7095 | CCAAGAAU A UAUGCUAC | 1231 | GTAGCATA GGCTAGCTACAACGA ATTCTTGG | 18024 |
| 7097 | AAGAAUAU A UGCUACAG | 1232 | CTGTAGCA GGCTAGCTACAACGA ATATTCTT | 18025 |
| 7099 | GAAUAUAU G CUACAGAU | 8277 | ATCTGTAG GGCTAGCTACAACGA ATATATTC | 18026 |
| 7102 | UAUAUGCU A CACAUAUA | 1233 | TATATCTG GGCTAGCTACAACGA AGCATATA | 18027 |
| 7106 | UGCUACAG A UAUAAGAC | 8278 | GTCTTATA GGCTAGCTACAACGA CTGTACCA | 18028 |
| 7108 | CUACAGAU A UAAGACAG | 1234 | CTGTCTTA GGCTAGCTACAACGA ATCTGTAG | 18029 |
| 7113 | GAUAUAAG A CAGACAUG | 8279 | CATGTCTG GGCTAGCTACAACGA CTTATATC | 18030 |
| 7117 | UAAGACAG A CAUGGUUU | 8280 | AAACCATG GGCTAGCTACAACGA CTGTCTTA | 18031 |
| 7119 | AGACAGAC A UGGUUUGG | 5552 | CCAAACCA GGCTAGCTACAACGA GTCTGTCT | 18032 |
| 7122 | CAGACAUG G UUUGGUCC | 8281 | GGACCAAA GGCTAGCTACAACGA CATGTCTG | 18033 |
| 7127 | AUGGUUUG G UCCUAUAU | 8282 | ATATAGGA GGCTAGCTACAACGA CAAACCAT | 18034 |
| 7132 | UUGGUCCU A UAUUUCUA | 1239 | TAGAAATA GGCTAGCTACAACGA AGGACCAA | 18035 |
| 7134 | GGUCCUAU A UUUCUAGU | 1240 | ACTAGAAA GGCTAGCTACAACGA ATAGGACC | 18036 |
| 7141 | UAUUUCUA G UGAUGAUG | 8283 | CATCATGA GGCTAGCTACAACGA TAGAAATA | 18037 |
| 7144 | UUCUAGUC A UGAUGAAU | 5556 | ATTCATCA GGCTAGCTACAACGA GACTAGAA | 18038 |
| 7147 | UAGUGAUG A UGAAUGUA | 8284 | TACATTCA GGCTAGCTACAACGA CATGACTA | 18039 |
| 7151 | CAUGAUGA A UGUAUUUU | 8285 | AAAATACA GGCTAGCTACAACGA TCATCATG | 18040 |
| 7153 | UGAUGAAU G UAUUUUGU | 8286 | ACAAAATA GGCTAGCTACAACGA ATTCATCA | 18041 |
| 7155 | AUGAAUGU A UUUUGUAU | 1246 | ATACAAAA GGCTAGCTACAACGA ACATTCAT | 18042 |
| 7160 | UGUAUUUU G UAUACCAU | 8287 | ATGGTATA GGCTAGCTACAACGA AAAATACA | 18043 |
| 7162 | UAUUUUGU A UACCAUCU | 1250 | AGATGGTA GGCTAGCTACAACGA ACAAAATA | 18044 |
| 7164 | UUUUGUAU A CCAUCUUC | 1251 | GAAGATGG GGCTAGCTACAACGA ATACAAAA | 18045 |
| 7167 | UGUAUACC A UCUUCAUA | 5558 | TATGAAGA GGCTAGCTACAACGA GUTATACA | 18046 |
| 7173 | CCAUCUUC A UAUAAUAU | 5560 | ATATTATA GGCTAGCTACAACGA GAAGATGG | 18047 |
| 7175 | AUCUUCAU A UAAUAUAC | 1255 | GTATATTA GGCTAGCTACAACGA ATGAAGAT | 18048 |
| 7178 | UUCAUAUA A UAUACUUA | 8288 | TAAGTATA GGCTAGCTACAACGA TATATGAA | 18049 |
| 7180 | CAUAUAAU A UACUUAAA | 1257 | TTTAAGTA GGCTAGCTACAACGA ATTATATG | 18050 |
| 7182 | UAUAAUAU A CUUAAAAA | 1258 | TTTTTAAG GGCTAGCTACAACGA ATATTATA | 18051 |
| 7190 | ACUUAAAA A UAUUUCUU | 8289 | AAGAAATA GGCTAGCTACAACGA TTTTAAGT | 18052 |
| 7192 | UUAAAAAU A UUUCUUAA | 1261 | TTAAGAAA GGCTAGCTACAACGA ATTTTTAA | 18053 |
| 7200 | AUUUCUUA A UUGGGAUU | 8290 | AATCCCAA GGCTAGCTACAACGA TAAGAAAT | 18054 |
| 7206 | UAAUUGGG A UUUGUAAU | 8291 | ATTACAAA GGCTAGCTACAACGA CCCAATTA | 18055 |
| 7210 | UGGGAUUU G UAAUCGUA | 8292 | TACGATTA GGCTAGCTACAACGA AAATCCCA | 18056 |
| 7213 | GAUUUGUA A UCCUACCA | 8293 | TGGTACGA GGCTAGCTACAACGA TACAAATC | 18057 |
| 7216 | UUGUAAUC G UACCAACU | 8294 | AGTTGGTA GGCTAGCTACAACGA GATTACAA | 18058 |
| 7218 | GUAAUCGU A CCAACUUA | 1272 | TAAGTTGG GGCTAGCTACAACGA ACGATTAC | 18059 |
| 7222 | UCGUACCA A CUUAAUUG | 8295 | CAATTAAG GGCTAGCTACAACGA TGGTACGA | 18060 |
| 7227 | CCAACUUA A UUGAUAAA | 8296 | TTTATCAA GGCTAGCTACAACGA TAAGTTGG | 18061 |
| 7231 | CUUAAUUG A UAAACUUG | 8297 | CAAGTTTA GGCTAGCTACAACGA CAATTAAG | 18062 |
| 7235 | AUUGAUAA A CUUGGCAA | 8298 | TTGCCAAG GGCTAGCTACAACGA TTATCAAT | 18063 |
| 7240 | UAAACUUG G CAACUGCU | 8299 | AGCAGTTG GGCTAGCTACAACGA CAAGTTTA | 18064 |
| 7243 | ACUUGGCA A CUGCUUUU | 8300 | AAAAGCAG GGCTAGCTACAACGA TGCCAAGT | 18065 |
| 7246 | UGGCAACU G CUUUUAUG | 8301 | CATAAAAG GGCTAGCTACAACGA AGTTGCCA | 18066 |
| 7252 | CUGCUUUU A UGUUCUGU | 1281 | ACAGAACA GGCTAGCTACAACGA AAAAGCAG | 18067 |
| 7254 | GCUUUUAU G UUCUGUCU | 8302 | AGACAGAA GGCTAGCTACAACGA ATAAAAGC | 18068 |
| 7259 | UAUGUUCU G UCUCCUUC | 8303 | GAAGGAGA GGCTAGCTACAACGA AGAACATA | 18069 |
| 7269 | CUCCUUCC A UAAAUUUU | 5575 | AAAATTTA GGCTAGCTACAACGA GGAAGGAG | 18070 |
| 7273 | UUCCAUAA A UUUUUCAA | 8304 | TTGAAAAA GGCTAGCTACAACGA TTATCGAA | 18071 |
| 7283 | UUUUCAAA A UACUAAUU | 8305 | AATTAGTA GGCTAGCTACAACGA TTTGAAAA | 18072 |
| 7285 | UUCAAAAU A CUAAUUCA | 1294 | TGAATTAG GGCTAGCTACAACGA ATTTTGAA | 18073 |
| 7289 | AAAUACUA A UUCAACAA | 8306 | TTCTTCAA GGCTAGCTACAACGA TAGTATTT | 18074 |
| 7294 | CUAAUUCA A CAAAGAAA | 8307 | TTTCTTTG GGCTAGCTACAACGA TGAATTAG | 18075 |
| 7305 | AAGAAAAA G CUCUUUUU | 8308 | AAAAAGAG GGCTAGCTACAACGA TTTTTCTT | 18076 |
| 7323 | UUCCUAAA A UAAACUCA | 8309 | TGAGTTTA GGCTAGCTACAACGA TTTAGGAA | 18077 |
| 7327 | UAAAAUAA A CUCAAAUU | 8310 | AATTTGAG GGCTAGCTACAACGA TTATTTTA | 18078 |
| 7333 | AAACUCAA A UUUAUCCU | 8311 | AGCATAAA GGCTAGCTACAACGA TTGAGTTT | 18079 |
| 7337 | UCAAAUUU A UCCUUGUU | 1312 | AACAAGGA GGCTAGCTACAACGA AAATTTGA | 18080 |
| 7343 | UUAUCCUU G UUUAGAGC | 8312 | GCTCTAAA GGCTAGCTACAACGA AAGUATAA | 18081 |
| 7350 | UGUUUAGA G CAGAGAAA | 8313 | TTTCTCTG GGCTAGCTACAACGA TCTAAACA | 18082 |
| 7360 | AGAGAAAA A UUAAGAAA | 8314 | TTTCTTAA GGCTAGCTACAACGA TTTTCTCT | 18083 |
| 7370 | UAACAAAA A CUUUGAAA | 8315 | TTTCAAAG GGCTAGCTACAACGA TTTTCTTA | 18084 |
| 7378 | ACUUUGAA A UGGUCUCA | 8316 | TGAGACCA GGCTAGCTACAACGA TTCAAAGT | 18085 |
| 7381 | UUGAAAUG G UCUCAAAA | 8317 | TTTTGAUA GGCTAGCTACAACGA CATTTCAA | 18086 |
| 7391 | CUCAAAAA A UUGCUAAA | 8318 | TTTAGCAA GGCTAGCTACAACGA TTTTTGAG | 18087 |
| 7394 | AAAAAAUU G CUAAAUAU | 8319 | ATATTTAU GGCTAGCTACAACGA AATTTTTT | 18088 |
| 7399 | AUUGCUAA A UAUUUUCA | 8320 | TGAAAATA GGCTAGCTACAACGA TTAGCAAT | 18089 |
| 7401 | UUCUAAAU A UUUUCAAU | 1326 | ATTGAAAA GGCTAGCTACAACGA ATTTAGCA | 18090 |
| 7408 | UAUUUUCA A UGGAAAAC | 8321 | UTTTTCCA GGCTAGCTACAACGA TUAAAATA | 18091 |
| 7415 | AAUUGAAA A CUAAAUUU | 8322 | ACATTTAG GGCTAGCTACAACGA TTTCCATT | 18092 |
| 7420 | AAAACUAA A UGUUAGUU | 8323 | AACTAACA GGCTAGCTACAACGA TTAGTTTT | 18093 |
| 7422 | AACUAAAU G UUAGUUUA | 8324 | TAAACTAA GGCTAGCTACAACGA ATTTAGTT | 18094 |
| 7426 | AAAUGUUA G HUUAUCUG | 8325 | CAGCTAAA GGCTAGCTACAACGA TAACATTT | 18095 |
| 7431 | UUAGUUUA G CUGAUUGU | 8326 | ACAATCAG GGCTAGCTACAACGA TAAACTAA | 18096 |
| 7435 | UUUAGCUG A UUGUAUGG | 8327 | CCATACAA GGCTAGCTACAACGA CAGCTAAA | 18097 |
| 7438 | AGCUUAUU G UAUGGGGU | 8328 | ACCCCATA GGCTAGCTACAACGA AATCAGCT | 18098 |
| 7440 | CUGAUUGU A UGGGGUUU | 1338 | AAACCCCA GGCTAGCTACAACGA ACAATCAG | 18099 |
| 7445 | UGUAUGGG G UUUUCGAA | 8329 | TTCGAAAA GGCTAGCTACAACGA CCCATACA | 18100 |
| 7453 | GUUUUCGA A CCUUUCAC | 8330 | GTGAAAGG GGCTAGCTACAACGA TCGAAAAC | 18101 |
| 7460 | AACCUUUC A CUUUUUGU | 5598 | ACAAAAAG GGCTAGCTACAACGA GAAAGGTT | 18102 |
| 7467 | CACUUUUU G UUUGUUUU | 8331 | AAAACAAA GGCTAGCTACAACGA AAAAAGTG | 18103 |
| 7471 | UUUUGUUU G UUUUACCU | 8332 | AGGTAAAA GGCTAGCTACAACGA AAACAAAA | 18104 |
| 7476 | UUUGUUUU A CCUAUUUC | 1355 | GAAATAGG GGCTAGCTACAACGA AAAACAAA | 18105 |
| 7480 | UUUUACCU A UUUCACAA | 1356 | TTGTGAAA GGCTAGCTACAACGA AGGTAAAA | 18106 |
| 7485 | CCUAUUUC A CAACUGUG | 5602 | CACAGTTG GGCTAGCTACAACGA GAAATAGG | 18107 |
| 7488 | AUUUCACA A CUGUGUAA | 8333 | TTACACAG GGCTAGCTACAACGA TGTGAAAT | 18108 |
| 7491 | UCACAACU G UGUAAAUU | 8334 | AATTTACA GGCTAGCTACAACGA AUTTGTGA | 18109 |
| 7493 | ACAACUGU G UAAAUUGC | 8335 | UCAATTTA GGCTAGCTACAACGA ACAGTTGT | 18110 |
| 7497 | CUUUGUAA A UUGCCAAU | 8336 | ATTGGCAA GGCTAUCTACAACGA TTACACAG | 18111 |
| 7500 | UUUAAAUU G CCAAUAAU | 8337 | ATTATTGU GGCTAUCTACAACGA AATTTACA | 18112 |
| 7504 | AAUUGCCA A UAAUUCCU | 8338 | AGGAATTA GGCTAGCTACAACGA TGGCAATT | 18113 |
| 7507 | UGCCAAUA A UUCCUGUC | 8339 | GACAGGAA GGCTAGCTACAACGA TATTGGCA | 18114 |
| 7513 | UAAUUCCU G UCCAUGAA | 8340 | TTCATGGA GGCTAGCTACAACGA AGGAATTA | 18115 |
| 7517 | UCCUGUCC A UGAAAAUG | 5610 | CATTTTCA GGCTAGCTACAACGA GGACAGGA | 18116 |
| 7523 | CCAUGAAA A UGCAAAUU | 8341 | AATTTGCA GGCTAGCTACAACGA TTTCATGC | 18117 |
| 7525 | AUGAAAAU G CAAAUUAU | 8342 | ATAATTTG GGCTAGCTACAACGA ATTTTCAT | 18118 |
| 7529 | AAAUGCAA A UUAUCCAG | 8343 | CTGCATAA GGCTAGCTACAACGA TTGCATTT | 18119 |
| 7532 | UGCAAAUU A UCCAGUGU | 1367 | ACACTGGA GGCTAGCTACAACGA AATTTGCA | 18120 |
| 7537 | AUUAUCCA G UCUAGAUA | 8344 | TATCTACA GGCTAGCTACAACGA TGGATAAT | 18121 |
| 7539 | UAUCCAGU G UAGAUAUA | 8345 | TATATCTA GGCTAGCTACAACGA ACTCGATA | 18122 |
| 7543 | CACUCUAG A UAUAUUUC | 8346 | CAAATATA GGCTAGCTACAACGA CTACACTC | 18123 |
| 7545 | GUCUAGAU A UAUUUCAC | 1370 | GTCAAATA GGCTAGCTACAACGA ATCTACAC | 18124 |
| 7547 | CUAGAUAU A UUUGACCA | 1371 | TGCTCAAA GGCTAGCTACAACGA ATATCTAC | 18125 |
| 7552 | UAUAUUUG A CCAUCACC | 8347 | GCTCATGC GGCTAGCTACAACGA CAAATATA | 18126 |
| 7555 | AUUUCACC A UCACCCUA | 5615 | TAGCCTGA GGCTAGCTACAACGA CCTCAAAT | 18127 |
| 7558 | UGACCAUC A CCCUAUCC | 5616 | CCATACCG GGCTAGCTACAACGA GATCGTCA | 18128 |
| 7563 | AUCACCCU A UCCAUAUU | 1375 | AATATCCA GGCTAGCTACAACGA AGGGTCAT | 18129 |
| 7567 | CCCUAUGG A UAUUCGCU | 8348 | ACCCAATA GGCTAGCTACAACGA CCATAGGG | 18130 |
| 7569 | CUAUGCAU A UUGGCUAC | 1376 | CTAGCCAA GGCTAGCTACAACGA ATCCATAG | 18131 |
| 7573 | GGAUAUUC G CUACUUUU | 8349 | AAAACTAC GGCTAGCTACAACGA CAATATCC | 18132 |
| 7577 | AUUCCCUA G UUUUCCCU | 8350 | AGGCAAAA GGCTAGCTACAACGA TAGCCAAT | 18133 |
| 7582 | CUACUUUU G CCUUUAUU | 8351 | AATAAAGC GGCTAGCTACAACGA AAAACTAC | 18134 |
| 7588 | UUCCCUUU A UUAAGCAA | 1384 | TTCCTTAA GGCTAGCTACAACGA AAACGCAA | 18135 |
| 7593 | UUUAUUAA G CAAAUUCA | 8352 | TGAATTTG GGCTAGCTACAACGA TTAATAAA | 18136 |
| 7597 | UUAAGCAA A UUCAUUUC | 8353 | CAAATGAA GGCTAGCTACAACGA TTCCTTAA | 18137 |
| 7601 | GCAAAUUC A UUUCACCC | 5624 | GCCTGAAA GGCTAGCTACAACGA GAATTTCC | 18138 |
| 7607 | UCAUUUCA G CCUGAAUC | 8354 | CATTCAGG GGCTAGCTACAACGA TGAAATGA | 18139 |
| 7613 | CAGCCUGA A UCUCUCCC | 8355 | GCCACACA GGCTAGCTACAACGA TCAGCCTG | 18140 |
| 7615 | CCCUCAAU G UCUCCCUA | 8356 | TAGGCAGA GGCTAGCTACAACGA ATTCACCC | 18141 |
| 7619 | GAAUCUCU G CCUAUAUA | 8357 | TATATAGC GGCTAGCTACAACGA ACACATTC | 18142 |
| 7623 | CUCUGCCU A UAUAUUCU | 1393 | AGAATATA GGCTAGCTACAACGA AGGCACAC | 18143 |
| 7625 | CUCCCUAU A UAUUCUCU | 1394 | AGAGAATA GGCTAGCTACAACGA ATAGCCAC | 18144 |
| 7627 | CCCUAUAU A UUCUCUGC | 1395 | CCAGAGAA GGCTAGCTACAACGA ATATACCC | 18145 |
| 7634 | UAUUCUCU G CUCUUUGU | 8358 | ACAAAGAG GGCTAGCTACAACGA AGAGAATA | 18146 |
| 7641 | UCCUCUUU G UAUUCUCC | 8359 | CGACAATA GGCTAGCTACAACGA AAACAGCA | 18147 |
| 7643 | CUCUUUCU A UUCUCCUU | 1402 | AACCACAA GGCTAGCTACAACGA ACAAACAC | 18148 |
| 7655 | UCCUUUCA A CCCCUUAA | 8360 | TTAACCCC GGCTAGCTACAACGA TCAAACCA | 18149 |
| 7659 | UUCAACCC G UUAAAACA | 8361 | TCTTTTAA GGCTAGCTACAACGA CCCTTCAA | 18150 |
| 7665 | CCCUUAAA A CAUCCUCU | 8362 | ACACCATC GGCTAGCTACAACGA TTTAACCC | 18151 |
| 7667 | CUUAAAAC A UCCUCUCC | 5640 | CCACACCA GGCTAGCTACAACGA CTTTTAAC | 18152 |
| 7672 | AACAUCCU G UCCCACUC | 8363 | CACTCCCA GGCTAGCTACAACGA ACCATCTT | 18153 |

Input Sequence = HSFLT.
Cut Site = R/Y
Arm Length = 8.
Core Sequence = GGCTAGCTACAACGA
HSFLT (Human flt mRNA for receptor-related tyrosine kinase.; Acc# X51602; 7680 bp)

TABLE XIX
Human FLT Zinzyme and Substrate Sequence

| | | Seq ID | | Seq ID |
|---|---|---|---|---|
| Pos | Substrate | No | Zinzyme | No |
| 17 | UCCUCUCG G CUCCUCCC | 7143 | GGGAGGAG GCCGAAAGGCGAGUGAGGUCU CGAGAGGA | 18154 |
| 28 | CCUCCCCG G CAGCGGCG | 7144 | CGCCGCUG GCCGAAAGGCGAGUGAGGUCU CGGGGAGG | 18155 |
| 31 | CCCCGGCA G CGGCGGCG | 7145 | CGCCGCCG GCCGAAAGGCGAGUGAGGUCU UGCCGGGG | 18156 |
| 34 | CGGCAGCG G CGGCGGCU | 7146 | AGCCGCCG GCCGAAAGGCGAGUGAGGUCU CGCUGCCG | 18157 |
| 37 | CAGCGGCG G CGGCUCGG | 7147 | CCGAGCCG GCCGAAAGGCGAGUGAGGUCU CGCCGCUG | 18158 |
| 40 | CGGCGGCG G CUCGGAGC | 7148 | GCUCCGAG GCCGAAAGGCGAGUGAGGUCU CGCCGCCG | 18159 |
| 47 | GGCUCGGA G CGGGCUCC | 7149 | GGAGCCCG GCCGAAAGGCGAGUGAGGUCU UCCGAGCC | 18160 |
| 51 | CGGAGCGG G CUCCGGGG | 7150 | CCCCGGAG GCCGAAAGGCGAGUGAGGUCU CCGCUCCG | 18161 |
| 59 | GCUCCGGG G CUCGGGUG | 7151 | CACCCGAG GCCGAAAGGCGAGUGAGGUCU CCCGGAGC | 18162 |
| 65 | GGGCUCGG G UGCAGCGG | 7152 | CCGCUGCA GCCGAAAGGCGAGUGAGGUCU CCGAGCCC | 18163 |
| 67 | GCUCGGGU G CAGCGGCC | 7153 | GGCCGCUG GCCGAAAGGCGAGUGAGGUCU ACCCGAGC | 18164 |
| 70 | CGGGUGCA G CGGCCAGC | 7154 | GCUGGCCG GCCGAAAGGCGAGUGAGGUCU UGCACCCG | 18165 |
| 73 | GUGCAGCG G CCAGCGGG | 7155 | CCCGCUGG GCCGAAAGGCGAGUGAGGUCU CGCUGCAC | 18166 |
| 77 | AGCGGCCA G CGGGCCUG | 7156 | CAGGCCCG GCCGAAAGGCGAGUGAGGUCU UGGCCGCU | 18167 |
| 81 | GCCAGCGG G CCUGGCGG | 7157 | CCGCCAGG GCCGAAAGGCGAGUGAGGUCU CCGCUGGC | 18168 |
| 86 | CGGGCCUG G CGGCGAGG | 7158 | CCUCGCCG GCCGAAAGGCGAGUGAGGUCU CAGGCCCG | 18169 |
| 89 | GCCUGGCG G CGAGGAUU | 7159 | AAUCCUCG GCCGAAAGGCGAGUGAGGUCU CGCCAGGC | 18170 |
| 108 | CCGGGGAA G UGGUUGUC | 7161 | GACAACCA GCCGAAAGGCGAGUGAGGUCU UUCCCCGG | 18171 |
| 111 | GGGAAGUG G UUGUCUCC | 7162 | GGAGACAA GCCGAAAGGCGAGUGAGGUCU CACUUCCC | 18172 |
| 114 | AAGUGGUU G UCUCCUGG | 7163 | CCAGGAGA GCCGAAAGGCGAGUGAGGUCU AACCACUU | 18173 |
| 122 | GUCUCCUG G CUGGAGCC | 7164 | GGCUCCAG GCCGAAAGGCGAGUGAGGUCU CAGGAGAC | 18174 |
| 128 | UGGCUGGA G CCGCGAGA | 7165 | UCUCGCGG GCCGAAAGGCGAGUGAGGUCU UCCAGCCA | 18175 |
| 131 | CUGGAGCC G CGAGACGG | 7166 | CCGUCUCG GCCGAAAGGCGAGUGAGGUCU GGCUCCAG | 18176 |
| 140 | CGAGACGG G CGCUCAGG | 7168 | CCUGAGCG GCCGAAAGGCGAGUGAGGUCU CCGUCUCG | 18177 |
| 142 | AGACGGGC G CUCAGGGC | 7169 | GCCCUGAG GCCGAAAGGCGAGUGAGGUCU GCCCGUCU | 18178 |
| 149 | CGCUCAGG G CGCGGGGC | 7170 | GCCCCGCG GCCGAAAGGCGAGUGAGGUCU CCUGAGCG | 18179 |
| 151 | CUCAGGGC G CGGGGCCG | 7171 | CGGCCCCG GCCGAAAGGCGAGUGAGGUCU GCCCUGAG | 18180 |
| 156 | GGCGCGGG G CCGGCGGC | 7172 | GCCGCCGG GCCGAAAGGCGAGUGAGGUCU CCCGCGCC | 18181 |
| 160 | CGGGGCCG G CGGCGGCG | 7173 | CGCCGCCG GCCGAAAGGCGAGUGAGGUCU CGGCCCCG | 18182 |
| 163 | GGCCGGCG G CGGCGAAC | 7174 | GUUCGCCG GCCGAAAGGCGAGUGAGGUCU CGCCGGCC | 18183 |
| 166 | CGGCGGCG G CGAACGAG | 7175 | CUCGUUCG GCCGAAAGGCGAGUGAGGUCU CGCCGCCG | 18184 |
| 188 | GGACUCUG G CGGCCGGG | 7179 | CCCGGCCG GCCGAAAGGCGAGUGAGGUCU CAGAGUCC | 18185 |
| 191 | CUCUGGCG G CCGGGUCG | 7180 | CGACCCGG GCCGAAAGGCGAGUGAGGUCU CGCCAGAG | 18186 |
| 196 | GCGGCCGG G UCGUUGGC | 7181 | GCCAACGA GCCGAAAGGCGAGUGAGGUCU CCGGCCGC | 18187 |
| 199 | GCCGGGUC G UUGGCCGG | 7182 | CCGGCCAA GCCGAAAGGCGAGUGAGGUCU GACCCGGC | 18188 |
| 203 | GGUCGUUG G CCGGGGGA | 7183 | UCCCCCGG GCCGAAAGGCGAGUGAGGUCU CAACGACC | 18189 |
| 212 | CCGGGGGA G CGCGGGCA | 7184 | UGCCCGCG GCCGAAAGGCGAGUGAGGUCU UCCCCCGG | 18190 |
| 214 | GGGGGAGC G CGGGCACC | 7185 | GGUGCCCG GCCGAAAGGCGAGUGAGGUCU GCUCCCCC | 18191 |
| 218 | GAGCGCGG G CACCGGGC | 7186 | GCCCGGUG GCCGAAAGGCGAGUGAGGUCU CCGCGCUC | 18192 |
| 225 | GGCACCGG G CGAGCAGG | 7187 | CCUGCUCG GCCGAAAGGCGAGUGAGGUCU CCGGUGCC | 18193 |
| 229 | CCGGGCGA G CAGGCCGC | 7188 | GCGGCCUG GCCGAAAGGCGAGUGAGGUCU UCGCCCGG | 18194 |
| 233 | GCGAGCAG G CCGCGUCG | 7189 | CGACGCGG GCCGAAAGGCGAGUGAGGUCU CUGCUCGC | 18195 |
| 236 | AGCAGGCC G CGUCGCGC | 7190 | GCGCGACG GCCGAAAGGCGAGUGAGGUCU GGCCUGCU | 18196 |
| 238 | CAGGCCGC G UCGCGCUC | 7191 | GAGCGCGA GCCGAAAGGCGAGUGAGGUCU GCGGCCUG | 18197 |
| 241 | GCCGCGUC G CGCUCACC | 7192 | GGUGAGCG GCCGAAAGGCGAGUGAGGUCU GACGCGGC | 18198 |
| 243 | CGCGUCGC G CUCACCAU | 7193 | AUGGUGAG GCCGAAAGGCGAGUGAGGUCU GCGACGCG | 18199 |
| 253 | UCACCAUG G UCAGCUAC | 7194 | GUAGCUGA GCCGAAAGGCGAGUGAGGUCU CAUGGUGA | 18200 |
| 257 | CAUGGUCA G CUACUGGG | 7195 | CCCAGUAG GCCGAAAGGCGAGUGAGGUCU UGACCAUG | 18201 |
| 274 | ACACCGGG G UCCUGCUG | 7197 | CAGCAGGA GCCGAAAGGCGAGUGAGGUCU CCCGGUGU | 18202 |
| 279 | GGGGUCCU G CUGUGCGC | 7198 | GCGCACAG GCCGAAAGGCGAGUGAGGUCU AGGACCCC | 18203 |
| 282 | GUCCUGCU G UGCGCGCU | 7199 | AGCGCGCA GCCGAAAGGCGAGUGAGGUCU UGACCAUG | 18204 |
| 284 | CCUGCUGU G CGCGCUGC | 7200 | GCAGCGCG GCCGAAAGGCGAGUGAGGUCU ACAGCAGG | 18205 |
| 286 | UGCUGUGC G CGCUGCUC | 7201 | GAGCAGCG GCCGAAAGGCGAGUGAGGUCU GCACAGCA | 18206 |
| 288 | CUGUGCGC G CUGCUCAG | 7202 | CUGAGCAG GCCGAAAGGCGAGUGAGGUCU GCGCACAG | 18207 |
| 291 | UGCGCGCU G CUCAGCUG | 7203 | CAGCUGAG GCCGAAAGGCGAGUGAGGUCU AGCGCGCA | 18208 |
| 296 | GCUGCUCA G CUGUCUGC | 7204 | GCAGACAG GCCGAAAGGCGAGUGAGGUCU UGAGCAGC | 18209 |
| 299 | GCUCAGCU G UCUGCUUC | 7205 | GAAGCAGA GCCGAAAGGCGAGUGAGGUCU AGCUGAGC | 18210 |
| 303 | AGCUGUCU G CUUCUCAC | 7206 | GUGAGAAG GCCGAAAGGCGAGUGAGGUCU UCAGUUCA | 18211 |
| 320 | AGGAUCUA G UUCAGGUU | 7208 | AACCUGAA GCCGAAAGGCGAGUGAGGUCU UAGAUCCU | 18212 |
| 326 | UAGUUCAG G UUCAAAAU | 7209 | AUUUUGAA GCCGAAAGGCGAGUGAGGUCU CUGAACUA | 18213 |
| 353 | UGAACUGA G UUUAAAAG | 7213 | CUUUUAAA GCCGAAAGGCGAGUGAGGUCU UCAGUUCA | 18214 |
| 362 | UUUAAAAG G CACCCAGC | 7214 | GCUGGGUG GCCGAAAGGCGAGUGAGGUCU CUUUUAAA | 18215 |
| 369 | GGCACCCA G CACAUCAU | 7215 | AUGAUGUG GCCGAAAGGCGAGUGAGGUCU UGGGUGCC | 18216 |
| 378 | CACAUCAU G CAAGCAGG | 7216 | CCUGCUUG GCCGAAAGGCGAGUGAGGUCU AUGAUGUG | 18217 |
| 382 | UCAUGCAA G CAGGCCAG | 7217 | CUGGCCUG GCCGAAAGGCGAGUGAGGUCU UUGCAUGA | 18218 |
| 386 | GCAAGCAG G CCAGACAC | 7218 | GUGUCUGG GCCGAAAGGCGAGUGAGGUCU CUGCUUGC | 18219 |
| 396 | CAGACACU G CAUCUCCA | 7220 | UGGAGAUG GCCGAAAGGCGAGUGAGGUCU AGUGUCUG | 18220 |
| 407 | UCUCCAAU G CAGGGGGG | 7222 | CCCCCCUG GCCGAAAGGCGAGUGAGGUCU AUUGGAGA | 18221 |
| 418 | GGGGGGAA G CAGCCCAU | 7223 | AUGGGCUG GCCGAAAGGCGAGUGAGGUCU UUCCCCCC | 18222 |
| 421 | GGGAAGCA G CCCAUAAA | 7224 | UUUAUGGG GCCGAAAGGCGAGUGAGGUCU UGCUUCCC | 18223 |
| 432 | CAUAAAUG G UCUUUGCC | 7226 | GGCAAAGA GCCGAAAGGCGAGUGAGGUCU CAUUUAUG | 18224 |
| 438 | UGGUCUUU G CCUGAAAU | 7227 | AUUUCAGG GCCGAAAGGCGAGUGAGGUCU AAAGACCA | 18225 |
| 448 | CUGAAAUG G UGAGUAAG | 7229 | CUUACUCA GCCGAAAGGCGAGUGAGGUCU CAUUUCAG | 18226 |
| 452 | AAUGGUGA G UAAGGAAA | 7230 | UUUCCUUA GCCGAAAGGCGAGUGAGGUCU UCACCAUU | 18227 |
| 461 | UAAGGAAA G CGAAAGGC | 7231 | GCCUUUCG GCCGAAAGGCGAGUGAGGUCU UUUCCUUA | 18228 |
| 468 | AGCGAAAG G CUGAGCAU | 7232 | AUGCUCAG GCCGAAAGGCGAGUGAGGUCU CUUUCGCU | 18229 |
| 473 | AAGGCUGA G CAUAACUA | 7233 | UAGUUAUG GCCGAAAGGCGAGUGAGGUCU UCAGCCUU | 18230 |
| 487 | CUAAAUCU G CCUGUGGA | 7236 | UCCACAGG GCCGAAAGGCGAGUGAGGUCU AGAUUUAG | 18231 |
| 491 | AUCUGCCU G UGGAAGAA | 7237 | UUCUUCCA GCCGAAAGGCGAGUGAGGUCU AGGCAGAU | 18232 |
| 503 | AAGAAAUG G CAAACAAU | 7239 | AUUGUUUG GCCGAAAGGCGAGUGAGGUCU CAUUUCUU | 18233 |
| 515 | ACAAUUCU G CAGUACUU | 7242 | AAGUACUG GCCGAAAGGCGAGUGAGGUCU AGAAUUGU | 18234 |
| 518 | AUUCUGCA G UACUUUAA | 7243 | UUAAAGUA GCCGAAAGGCGAGUGAGGUCU UGCAGAAU | 18235 |
| 538 | UGAACACA G CUCAAGCA | 7246 | UGCUUGAG GCCGAAAGGCGAGUGAGGUCU UGUGUUCA | 18236 |
| 544 | CAGCUCAA G CAAACCAC | 7247 | GUGGUUUG GCCGAAAGGCGAGUGAGGUCU UUGAGCUG | 18237 |
| 557 | CCACACUG G CUUCUACA | 7249 | UGUAGAAG GCCGAAAGGCGAGUGAGGUCU CAGUGUGG | 18238 |
| 566 | CUUCUACA G CUGCAAAU | 7250 | AUUUGCAG GCCGAAAGGCGAGUGAGGUCU UGUAGAAG | 18239 |
| 569 | CUACAGCU G CAAAUAUC | 7251 | GAUAUUUG GCCGAAAGGCGAGUGAGGUCU AGCUGUAG | 18240 |
| 580 | AAUAUCUA G CUGUACCU | 7253 | AGGUACAG GCCGAAAGGCGAGUGAGGUCU UAGAUAUU | 18241 |
| 583 | AUCUAGCU G UACCUACU | 7254 | AGUAGGUA GCCGAAAGGCGAGUGAGGUCU AGCUAGAU | 18242 |
| 616 | CAGAAUCU G CAAUCUAU | 7257 | AUAGAUUG GCCGAAAGGCGAGUGAGGUCU AGAUUCUG | 18243 |
| 635 | AUUUAUUA G UGAUACAG | 7259 | CUGUAUCA GCCGAAAGGCGAGUGAGGUCU UAAUAAAU | 18244 |
| 644 | UGAUACAG G UAGACCUU | 7261 | AAGGUCUA GCCGAAAGGCGAGUGAGGUCU CUGUAUCA | 18245 |
| 655 | GACCUUUC G UAGAGAUG | 7263 | CAUCUCUA GCCGAAAGGCGAGUGAGGUCU GAAAGGUC | 18246 |
| 663 | GUAGAGAU G UACAGUGA | 7265 | UCACUGUA GCCGAAAGGCGAGUGAGGUCU AUCUCUAC | 18247 |
| 668 | GAUGUACA G UGAAAUCC | 7266 | GGAUUUCA GCCGAAAGGCGAGUGAGGUCU UGUACAUC | 18248 |
| 708 | GGAAGGGA G CUCGUCAU | 7270 | AUGACGAG GCCGAAAGGCGAGUGAGGUCU UCCCUUCC | 18249 |
| 712 | GGGAGCUC G UCAUUCCC | 7271 | GGGAAUGA GCCGAAAGGCGAGUGAGGUCU GAGCUCCC | 18250 |
| 722 | CAUUCCCU G CCGGGUUA | 7272 | UAACCCGG GCCGAAAGGCGAGUGAGGUCU AGGGAAUG | 18251 |
| 727 | CCUGCCGG G UUACGUCA | 7273 | UGACGUAA GCCGAAAGGCGAGUGAGGUCU CCGGCAGG | 18252 |
| 732 | CGGGUUAC G UCACCUAA | 7274 | UUAGGUGA GCCGAAAGGCGAGUGAGGUCU GUAACCCG | 18253 |
| 748 | ACAUCACU G UUACUUUA | 7276 | UAAAGUAA GCCGAAAGGCGAGUGAGGUCU AGUGAUGU | 18254 |
| 762 | UUAAAAAA G UUUCCACU | 7277 | AGUGGAAA GCCGAAAGGCGAGUGAGGUCU UUUUUUAA | 18255 |
| 797 | UGGAAAAC G CAUAAUCU | 7282 | AGAUUAUG GCCGAAAGGCGAGUGAGGUCU GUUUUCCA | 18256 |
| 812 | CUGGGACA G UAGAAAGG | 7285 | CCUUUCUA GCCGAAAGGCGAGUGAGGUCU UGUCCCAG | 18257 |
| 821 | UAGAAAGG G CUUCAUCA | 7286 | UGAUGAAG GCCGAAAGGCGAGUGAGGUCU CCUUUCUA | 18258 |
| 838 | UAUCAAAU G CAACGUAC | 7288 | GUACGUUG GCCGAAAGGCGAGUGAGGUCU AUUUGAUA | 18259 |
| 843 | AAUGCAAC G UACAAAGA | 7290 | UCUUUGUA GCCGAAAGGCGAGUGAGGUCU GUUGCAUU | 18260 |
| 858 | GAAAUAGG G CUUCUGAC | 7292 | GUCAGAAG GCCGAAAGGCGAGUGAGGUCU CCUAUUUC | 18261 |
| 869 | UCUGACCU G UGAAGCAA | 7294 | UUGCUUCA GCCGAAAGGCGAGUGAGGUCU AGGUCAGA | 18262 |
| 874 | CCUGUGAA G CAACAGUC | 7295 | GACUGUUG GCCGAAAGGCGAGUGAGGUCU UUCACAGG | 18263 |
| 880 | AAGCAACA G UCAAUGGG | 7297 | CCCAUUGA GCCGAAAGGCGAGUGAGGUCU UGUUGCUU | 18264 |
| 888 | GUCAAUGG G CAUUUGUA | 7299 | UACAAAUG GCCGAAAGGCGAGUGAGGUCU CCAUUGAC | 18265 |
| 894 | GGGCAUUU G UAUAAGAC | 7300 | GUCUUAUA GCCGAAAGGCGAGUGAGGUCU AUCUAUGA | 18266 |
| 943 | UCAUAGAU G UCCAAAUA | 7308 | UAUUUGGA GCCGAAAGGCGAGUGAGGUCU AUCUAUGA | 18267 |
| 953 | CCAAAUAA G CACACCAC | 7310 | GUGGUGUG GCCGAAAGGCGAGUGAGGUCU UUAUUUGG | 18268 |
| 962 | CACACCAC G CCCAGUCA | 7311 | UGACUGGG GCCGAAAGGCGAGUGAGGUCU GUGGUGUG | 18269 |
| 967 | CACGCCCA G UCAAAUUA | 7312 | UAAUUUGA GCCGAAAGGCGAGUGAGGUCU UGGGCGUG | 18270 |
| 983 | ACUUAGAG G CCAUACUC | 7314 | GAGUAUGG GCCGAAAGGCGAGUGAGGUCU CUCUAAGU | 18271 |
| 994 | AUACUCUU G UCCUCAAU | 7315 | AUUGAGGA GCCGAAAGGCGAGUGAGGUCU AAGAGUAU | 18272 |
| 1004 | CCUCAAUU G UACUGCUA | 7317 | UAGCAGUA GCCGAAAGGCGAGUGAGGUCU AAUUGAGG | 18273 |
| 1009 | AUUGUACU G CUACCACU | 7318 | AGUGGUAG GCCGAAAGGCGAGUGAGGUCU AGUACAAU | 18274 |
| 1033 | ACACGAGA G UUCAAAUG | 7320 | CAUUUGAA GCCGAAAGGCGAGUGAGGUCU UCUCGUGU | 18275 |
| 1049 | GACCUGGA G UUACCCUG | 7323 | CAGGGUAA GCCGAAAGGCGAGUGAGGUCU UCCAGGUC | 18276 |
| 1075 | AUAAGAGA G CUUCCGUA | 7326 | UACGGAAG GCCGAAAGGCGAGUGAGGUCU UCUCUUAU | 18277 |
| 1081 | GAGCUUCC G UAAGGCGA | 7327 | UCGCCUUA GCCGAAAGGCGAGUGAGGUCU GGAAGCUC | 18278 |
| 1086 | UCCGUAAG G CGACGAAU | 7328 | AUUCGUCG GCCGAAAGGCGAGUGAGGUCU CUUACGGA | 18279 |
| 1103 | UGACCAAA G CAAUUCCC | 7332 | GGGAAUUG GCCGAAAGGCGAGUGAGGUCU UUUGGUCA | 18280 |
| 1114 | AUUCCCAU G CCAACAUA | 7334 | UAUGUUGG GCCGAAAGGCGAGUGAGGUCU AUGGGAAU | 18281 |
| 1130 | AUUCUACA G UGUUCUUA | 7336 | UAAGAACA GCCGAAAGGCGAGUGAGGUCU UGUAGAAU | 18282 |
| 1132 | UCUACAGU G UUCUUACU | 7337 | AGUAAGAA GCCGAAAGGCGAGUGAGGUCU ACUGUAGA | 18283 |
| 1152 | GACAAAAU G CAGAACAA | 7340 | UUGUUCUG GCCGAAAGGCGAGUGAGGUCU AUUUUGUC | 18284 |
| 1181 | UUAUACUU G UCGUGUAA | 7344 | UUACACGA GCCGAAAGGCGAGUGAGGUCU AAGUAUAA | 18285 |
| 1184 | UACUUGUC G UGUAAGGA | 7345 | UCCUUACA GCCGAAAGGCGAGUGAGGUCU GACAAGUA | 18286 |
| 1186 | CUUGUCGU G UAAGGAGU | 7346 | ACUCCUUA GCCGAAAGGCGAGUGAGGUCU ACGACAAG | 18287 |
| 1193 | UGUAAGGA G UGGACCAU | 7347 | AUGGUCCA GCCGAAAGGCGAGUGAGGUCU UCCUUACA | 18288 |
| 1213 | UCAAAUCU G UUAACACC | 7350 | GGUGUUAA GCCGAAAGGCGAGUGAGGUCU AGAUUUGA | 18289 |
| 1225 | ACACCUCA G UGCAUAUA | 7352 | UAUAUGCA GCCGAAAGGCGAGUGAGGUCU UGAGGUGU | 18290 |
| 1227 | ACCUCAGU G CAUAUAUA | 7353 | UAUAUAUG GCCGAAAGGCGAGUGAGGUCU ACUGAGGU | 18291 |
| 1243 | AUGAUAAA G CAUUCAUC | 7355 | GAUGAAUG GCCGAAAGGCGAGUGAGGUCU UUUAUCAU | 18292 |
| 1255 | UCAUCACU G UGAAACAU | 7356 | AUGUUUCA GCCGAAAGGCGAGUGAGGUCU AGUGAUGA | 18293 |
| 1272 | CGAAAACA G CAGGUGCU | 7359 | AGCACCUG GCCGAAAGGCGAGUGAGGUCU UGUUUUCG | 18294 |
| 1276 | AACAGCAG G UGCUUGAA | 7360 | UUCAAGCA GCCGAAAGGCGAGUGAGGUCU CUGCUGUU | 18295 |
| 1278 | CAGCAGGU G CUUGAAAC | 7361 | GUUUCAAG GCCGAAAGGCGAGUGAGGUCU ACCUGCUG | 18296 |
| 1288 | UUGAAACC G UAGCUGGC | 7363 | GCCAGCUA GCCGAAAGGCGAGUGAGGUCU GGUUUCAA | 18297 |
| 1291 | AAACCGUA G CUGGCAAG | 7364 | CUUGCCAG GCCGAAAGGCGAGUGAGGUCU UACGGUUU | 18298 |
| 1295 | CGUAGCUG G CAAGCGGU | 7365 | ACCGCUUG GCCGAAAGGCGAGUGAGGUCU CAGCUACG | 18299 |
| 1299 | GCUGGCAA G CGGUCUUA | 7366 | UAAGACCG GCCGAAAGGCGAGUGAGGUCU UUGCCAGC | 18300 |
| 1302 | GGCAAGCG G UCUUACCG | 7367 | CGGUAAGA GCCGAAAGGCGAGUGAGGUCU CGCUUGCC | 18301 |
| 1311 | UCUUACCG G CUCUCUAU | 7368 | AUAGAGAG GCCGAAAGGCGAGUGAGGUCU CGGUAAGA | 18302 |
| 1324 | CUAUGAAA G UGAAGGCA | 7369 | UGCCUUCA GCCGAAAGGCGAGUGAGGUCU UUUCAUAG | 18303 |
| 1330 | AAGUGAAG G CAUUUCCC | 7370 | GGGAAAUG GCCGAAAGGCGAGUGAGGUCU CUUCACUU | 18304 |
| 1341 | UUUCCCUC G CCGGAAGU | 7371 | ACUUCCGG GCCGAAAGGCGAGUGAGGUCU GAGGGAAA | 18305 |
| 1348 | CGCCGGAA G UUGUAUGG | 7372 | CCAUACAA GCCGAAAGGCGAGUGAGGUCU UUCCGGCG | 18306 |
| 1351 | CGGAAGUU G UAUGGUUA | 7373 | UAACCAUA GCCGAAAGGCGAGUGAGGUCU AACUUCCG | 18307 |
| 1356 | GUUGUAUG G UUAAAAGA | 7374 | UCUUUUAA GCCGAAAGGCGAGUGAGGUCU CAUACAAC | 18308 |
| 1368 | AAAGAUGG G UUACCUGC | 7376 | GCAGGUAA GCCGAAAGGCGAGUGAGGUCU CCAUCUUU | 18309 |
| 1375 | GGUUACCU G CGACUGAG | 7377 | CUCAGUCG GCCGAAAGGCGAGUGAGGUCU AGGUAACC | 18310 |
| 1390 | AGAAAUCU G CUCGCUAU | 7380 | AUAGCGAG GCCGAAAGGCGAGUGAGGUCU AGAUUUCU | 18311 |
| 1394 | AUCUGCUC G CUAUUUGA | 7381 | UCAAAUAG GCCGAAAGGCGAGUGAGGUCU GAGCAGAU | 18312 |
| 1406 | UUUGACUC G UGGCUACU | 7383 | AGUAGCCA GCCGAAAGGCGAGUGAGGUCU GAGUCAAA | 18313 |
| 1409 | GACUCGUG G CUACUCGU | 7384 | ACGAGUAG GCCGAAAGGCGAGUGAGGUCU CACGAGUC | 18314 |
| 1416 | GGCUACUC G UUAAUUAU | 7385 | AUAAUUAA GCCGAAAGGCGAGUGAGGUCU GAGUAGCC | 18315 |
| 1432 | UCAAGGAC G UAACUGAA | 7388 | UUCAGUUA GCCGAAAGGCGAGUGAGGUCU GUCCUUGA | 18316 |
| 1447 | AAGAGGAU G CAGGGAAU | 7391 | AUUCCCUG GCCGAAAGGCGAGUGAGGUCU AUCCUCUU | 18317 |
| 1467 | ACAAUCUU G CUGAGCAU | 7394 | AUGCUCAG GCCGAAAGGCGAGUGAGGUCU AAGAUUGU | 18318 |
| 1472 | CUUGCUGA G CAUAAAAC | 7395 | GUUUUAUG GCCGAAAGGCGAGUGAGGUCU UCAGCAAG | 18319 |
| 1482 | AUAAAACA G UCAAAUGU | 7397 | ACAUUUGA GCCGAAAGGCGAGUGAGGUCU UGUUUUAU | 18320 |
| 1489 | AGUCAAAU G UGUUUAAA | 7399 | UUUAAACA GCCGAAAGGCGAGUGAGGUCU AUUUGACU | 18321 |
| 1491 | UCAAAUGU G UUUAAAAA | 7400 | UUUUUAAA GCCGAAAGGCGAGUGAGGUCU ACAUUUGA | 18322 |
| 1507 | ACCUCACU G CCACUCUA | 7402 | UAGAGUGG GCCGAAAGGCGAGUGAGGUCU AGUGAGGU | 18323 |
| 1519 | CUCUAAUU G UCAAUGUG | 7404 | CACAUUGA GCCGAAAGGCGAGUGAGGUCU AAUUAGAG | 18324 |
| 1525 | UUGUCAAU G UGAAACCC | 7406 | GGGUUUCA GCCGAAAGGCGAGUGAGGUCU AUUGACAA | 18325 |
| 1549 | ACGAAAAG G CCGUGUCA | 7409 | UGACACGG GCCGAAAGGCGAGUGAGGUCU CUUUUCGU | 18326 |
| 1552 | AAAAGGCC G UGUCAUCG | 7410 | CGAUGACA GCCGAAAGGCGAGUGAGGUCU GGCCUUUU | 18327 |
| 1554 | AAGGCCGU G UCAUCGUU | 7411 | AACGAUGA GCCGAAAGGCGAGUGAGGUCU ACGGCCUU | 18328 |
| 1560 | GUGUCAUC G UUUCCAGA | 7412 | UCUGGAAA GCCGAAAGGCGAGUGAGGUCU GAUGACAC | 18329 |
| 1573 | CAGACCCG G CUCUCUAC | 7414 | GUAGAGAG GCCGAAAGGCGAGUGAGGUCU CGGGUCUG | 18330 |
| 1589 | CCCACUGG G CAGCAGAC | 7415 | GUCUGCUG GCCGAAAGGCGAGUGAGGUCU CCAGUGGG | 18331 |
| 1592 | ACUGGGCA G CAGACAAA | 7416 | UUUGUCUG GCCGAAAGGCGAGUGAGGUCU UGCCCAGU | 18332 |
| 1610 | CCUGACUU G UACCGCAU | 7420 | AUGCGGUA GCCGAAAGGCGAGUGAGGUCU AAGUCAGG | 18333 |
| 1615 | CUUGUACC G CAUAUGGU | 7421 | ACCAUAUG GCCGAAAGGCGAGUGAGGUCU GGUACAAG | 18334 |
| 1622 | CGCAUAUG G UAUCCCUC | 7422 | GAGGGAUA GCCGAAAGGCGAGUGAGGUCU CAUAUGCG | 18335 |
| 1644 | ACAAUCAA G UGGUUCUG | 7425 | CAGAACCA GCCGAAAGGCGAGUGAGGUCU UUGAUUGU | 18336 |
| 1647 | AUCAAGUG G UUCUGGCA | 7426 | UGCCAGAA GCCGAAAGGCGAGUGAGGUCU CACUUGAU | 18337 |
| 1653 | UGGUUCUG G CACCCCUG | 7427 | CAGGGGUG GCCGAAAGGCGAGUGAGGUCU CAGAACCA | 18338 |
| 1661 | GCACCCCU G UAACCAUA | 7428 | UAUGGUUA GCCGAAAGGCGAGUGAGGUCU AGGGGUGC | 18339 |
| 1681 | AUUCCGAA G CAAGGUGU | 7431 | ACACCUUG GCCGAAAGGCGAGUGAGGUCU UUCGGAAU | 18340 |
| 1686 | GAAGCAAG G UGUGACUU | 7432 | AAGUCACA GCCGAAAGGCGAGUGAGGUCU CUUGCUUC | 18341 |
| 1688 | AGCAAGGU G UGACUUUU | 7433 | AAAAGUCA GCCGAAAGGCGAGUGAGGUCU ACCUUGCU | 18342 |
| 1697 | UGACUUUU G UUCCAAUA | 7435 | UAUUGGAA GCCGAAAGGCGAGUGAGGUCU AAAAGUCA | 18343 |
| 1713 | AAUGAAGA G UCCUUUAU | 7438 | AUAAAGGA GCCGAAAGGCGAGUGAGGUCU UCUUCAUU | 18344 |
| 1729 | UCCUGGAU G CUGACAGC | 7440 | GCUGUCAG GCCGAAAGGCGAGUGAGGUCU AUCCAGGA | 18345 |
| 1736 | UGCUGACA G CAACAUGG | 7442 | CCAUGUUG GCCGAAAGGCGAGUGAGGUCU UGUCAGCA | 18346 |
| 1760 | AAUUGAGA G CAUCACUC | 7446 | GAGUGAUG GCCGAAAGGCGAGUGAGGUCU UCUCAAUU | 18347 |
| 1770 | AUCACUCA G CGCAUGGC | 7447 | GCCAUGCG GCCGAAAGGCGAGUGAGGUCU UGAGUGAU | 18348 |
| 1772 | CACUCAGC G CAUGGCAA | 7448 | UUGCCAUG GCCGAAAGGCGAGUGAGGUCU GCUGAGUG | 18349 |
| 1777 | AGCGCAUG G CAAUAAUA | 7449 | UAUUAUUG GCCGAAAGGCGAGUGAGGUCU CAUGCGCU | 18350 |
| 1804 | AUAAGAUG G CUAGCACC | 7454 | GGUGCUAG GCCGAAAGGCGAGUGAGGUCU CAUCUUAU | 18351 |
| 1808 | GAUGGCUA G CACCUUGG | 7455 | CCAAGGUG GCCGAAAGGCGAGUGAGGUCU UAGCCAUC | 18352 |
| 1816 | GCACCUUG G UUGUGGCU | 7456 | AGCCACAA GCCGAAAGGCGAGUGAGGUCU CAAGGUGC | 18353 |
| 1819 | CCUUGGUU G UGGCUGAC | 7457 | GUCAGCCA GCCGAAAGGCGAGUGAGGUCU AACCAAGG | 18354 |
| 1822 | UGGUUGUG G CUGACUCU | 7458 | AGAGUCAG GCCGAAAGGCGAGUGAGGUCU CACAACCA | 18355 |
| 1853 | CUACAUUU G CAUAGCUU | 7462 | AAGCUAUG GCCGAAAGGCGAGUGAGGUCU AAAUGUAG | 18356 |
| 1858 | UUUGCAUA G CUUCCAAU | 7463 | AUUGGAAG GCCGAAAGGCGAGUGAGGUCU UAUGCAAA | 18357 |
| 1870 | CCAAUAAA G UUGGGACU | 7465 | AGUCCCAA GCCGAAAGGCGAGUGAGGUCU UUUAUUGG | 18358 |
| 1879 | UUGGGACU G UGGGAAGA | 7467 | UCUUCCCA GCCGAAAGGCGAGUGAGGUCU AGUCCCAA | 18359 |
| 1895 | AAACAUAA G CUUUUAUA | 7469 | UAUAAAAG GCCGAAAGGCGAGUGAGGUCU UUAUGUUU | 18360 |
| 1912 | UCACAGAU G UGCCAAAU | 7471 | AUUUGGCA GCCGAAAGGCGAGUGAGGUCU AUCUGUGA | 18361 |
| 1914 | ACAGUAGU G CCAAAUGG | 7472 | CCAUUUGG GCCGAAAGGCGAGUGAGGUCU ACAUCUGU | 18362 |
| 1923 | CCAAAUGG G UUUCAUGU | 7474 | ACAUGAAA GCCGAAAGGCGAGUGAGGUCU CCAUUUGG | 18363 |
| 1930 | GGUUUCAU G UUAACUUG | 7475 | CAAGUUAA GCCGAAAGGCGAGUGAGGUCU AUGAAACC | 18364 |
| 1947 | GAAAAAAU G CCGACGGA | 7478 | UCCGUCGG GCCGAAAGGCGAGUGAGGUCU AUUUUUUC | 18365 |
| 1974 | CUGAAACU G UCUUGCAC | 7482 | GUGCAAGA GCCGAAAGGCGAGUGAGGUCU AGUUUCAG | 18366 |
| 1979 | ACUGUCUU G CACAGUUA | 7483 | UAACUGUG GCCGAAAGGCGAGUGAGGUCU AAGACAGU | 18367 |
| 1984 | CUUGCACA G UUAACAAG | 7484 | CUUGUUAA GCCGAAAGGCGAGUGAGGUCU UGUGCAAG | 18368 |
| 1992 | GUUAACAA G UUCUUAUA | 7486 | UAUAAGAA GCCGAAAGGCGAGUGAGGUCU UUGUUAAC | 18369 |
| 2008 | ACAGAGAC G UUACUUGG | 7488 | CCAAGUAA GCCGAAAGGCGAGUGAGGUCU GUCUCUGU | 18370 |
| 2025 | AUUUUACU G CGGACAGU | 7490 | ACUGUCCG GCCGAAAGGCGAGUGAGGUCU AGUAAAAU | 18371 |
| 2032 | UGCGGACA G UUAAUAAC | 7492 | GUUAUUAA GCCGAAAGGCGAGUGAGGUCU UGUCCGCA | 18372 |
| 2049 | AGAACAAU G CACUACAG | 7497 | CUGUAGUG GCCGAAAGGCGAGUGAGGUCU AUUGUUCU | 18373 |
| 2057 | GCACUACA G UAUUAGCA | 7498 | UGCUAAUA GCCGAAAGGCGAGUGAGGUCU UGUAGUGC | 18374 |
| 2063 | CAGUAUUA G CAAGCAAA | 7499 | UUUGCUUG GCCGAAAGGCGAGUGAGGUCU UAAUACUG | 18375 |
| 2067 | AUUAGCAA G CAAAAAAU | 7500 | AUUUUUUG GCCGAAAGGCGAGUGAGGUCU UUGCUAAU | 18376 |
| 2077 | AAAAAAUG G CCAUCACU | 7502 | AGUGAUGG GCCGAAAGGCGAGUGAGGUCU CAUUUUUU | 18377 |
| 2091 | ACUAAGGA G CACUCCAU | 7503 | AUGGAGUG GCCGAAAGGCGAGUGAGGUCU UCCUUAGU | 18378 |
| 2125 | UCAUGAAU G UUUCCCUG | 7506 | CAGGGAAA GCCGAAAGGCGAGUGAGGUCU AUUCAUGA | 18379 |
| 2133 | GUUUCCCU G CAAGAUUC | 7507 | GAAUCUUG GCCGAAAGGCGAGUGAGGUCU AGGGAAAC | 18380 |
| 2144 | AGAUUCAG G CACCUAUG | 7509 | CAUAGGUG GCCGAAAGGCGAGUGAGGUCU CUGAAUCU | 18381 |
| 2152 | GCACCUAU G CCUGCAGA | 7510 | UCUGCAGG GCCGAAAGGCGAGUGAGGUCU AUAGGUGC | 18382 |
| 2156 | CUAUGCCU G CAGAGCCA | 7511 | UGGCUCUG GCCGAAAGGCGAGUGAGGUCU AGGCAUAG | 18383 |
| 2161 | CCUGCAGA G CCAGGAAU | 7512 | AUUCCUGG GCCGAAAGGCGAGUGAGGUCU UCUGCAGG | 18384 |
| 2170 | CCAGGAAU G UAUACACA | 7514 | UGUGUAUA GCCGAAAGGCGAGUGAGGUCU AUUCCUGG | 18385 |
| 2227 | AUCAGGAA G CACCAUAC | 7519 | GUAUGGUG GCCGAAAGGCGAGUGAGGUCU UUCCUGAU | 18386 |
| 2241 | UACCUCCU G CGAAACCU | 7520 | AGGUUUCG GCCGAAAGGCGAGUGAGGUCU AGGAGGUA | 18387 |
| 2252 | AAACCUCA G UGAUCACA | 7522 | UGUGAUCA GCCGAAAGGCGAGUGAGGUCU UGAGGUUU | 18388 |
| 2263 | AUCACACA G UGGCCAUC | 7524 | GAUGGCCA GCCGAAAGGCGAGUGAGGUCU UGUGUGAU | 18389 |
| 2266 | ACACAGUG G CCAUCAGC | 7525 | GCUGAUGG GCCGAAAGGCGAGUGAGGUCU CACUGUGU | 18390 |
| 2273 | GGCCAUCA G CAGUUCCA | 7526 | UGGAACUG GCCGAAAGGCGAGUGAGGUCU UGAUGGCC | 18391 |
| 2276 | CAUCAGCA G UUCCACCA | 7527 | UGGUGGAA GCCGAAAGGCGAGUGAGGUCU UGCUGAUG | 18392 |
| 2294 | UUUAGACU G UCAUGCUA | 7529 | UAGCAUGA GCCGAAAGGCGAGUGAGGUCU AGUCUAAA | 18393 |
| 2299 | ACUGUCAU G CUAAUGGU | 7530 | ACCAUUAG GCCGAAAGGCGAGUGAGGUCU AUGACAGU | 18394 |
| 2306 | UGCUAAUG G UGUCCCCG | 7532 | CGGGGACA GCCGAAAGGCGAGUGAGGUCU CAUUAGCA | 18395 |
| 2308 | CUAAUGGU G UCCCCGAG | 7533 | CUCGGGGA GCCGAAAGGCGAGUGAGGUCU ACCAUUAG | 18396 |
| 2316 | GUCCCCGA G CCUCAGAU | 7534 | AUCUGAGG GCCGAAAGGCGAGUGAGGUCU UCGGGGAC | 18397 |
| 2331 | AUCACUUG G UUUAAAAA | 7536 | UUUUUAAA GCCGAAAGGCGAGUGAGGUCU CAAGUGAU | 18398 |
| 2361 | CAACAAGA G CCUGGAAU | 7541 | AUUCCAGG GCCGAAAGGCGAGUGAGGUCU UCUUGUUG | 18399 |
| 2387 | ACCAGGAA G CAGCACGC | 7544 | GCGUGCUG GCCGAAAGGCGAGUGAGGUCU UUCCUGGU | 18400 |
| 2390 | AGGAAGCA G CACGCUGU | 7545 | ACAGCGUG GCCGAAAGGCGAGUGAGGUCU UGCUUCCU | 18401 |
| 2394 | AGCAGCAC G CUGUUUAU | 7546 | AUAAACAG GCCGAAAGGCGAGUGAGGUCU GUGCUGCU | 18402 |
| 2397 | AGCACGCU G UUUAUUGA | 7547 | UCAAUAAA GCCGAAAGGCGAGUGAGGUCU AGCGUGCU | 18403 |
| 2410 | UUGAAAGA G UCACAGAA | 7548 | UUCUGUGA GCCGAAAGGCGAGUGAGGUCU UCUUUCAA | 18404 |
| 2429 | GGAUGAAG G UGUCUAUC | 7550 | GAUAGACA GCCGAAAGGCGAGUGAGGUCU CUUCAUCC | 18405 |
| 2431 | AUGAAGGU G UCUAUCAC | 7551 | GUGAUAGA GCCGAAAGGCGAGUGAGGUCU ACCUUCAU | 18406 |
| 2441 | CUAUCACU G CAAAGCCA | 7552 | UGGCUUUG GCCGAAAGGCGAGUGAGGUCU AGUGAUAG | 18407 |
| 2446 | ACUGCAAA G CCACCAAC | 7553 | GUUGGUGG GCCGAAAGGCGAGUGAGGUCU UUUGCAGU | 18408 |
| 2462 | CCAGAAGG G CUCUGUGG | 7555 | CCACAGAG GCCGAAAGGCGAGUGAGGUCU CCUUCUGG | 18409 |
| 2467 | AGGGCUCU G UGGAAAGU | 7556 | ACUUUCCA GCCGAAAGGCGAGUGAGGUCU AGAGCCCU | 18410 |
| 2474 | UGUGGAAA G UUCAGCAU | 7557 | AUGCUGAA GCCGAAAGGCGAGUGAGGUCU UUUCCACA | 18411 |
| 2479 | AAAGUUCA G CAUACCUC | 7558 | GAGGUAUG GCCGAAAGGCGAGUGAGGUCU UGAACUUU | 18412 |
| 2491 | ACCUCACU G UUCAAGGA | 7559 | UCCUUGAA GCCGAAAGGCGAGUGAGGUCU AGUGAGGU | 18413 |
| 2511 | UCGGACAA G UCUAAUCU | 7562 | AGAUUAGA GCCGAAAGGCGAGUGAGGUCU UUGUCCGA | 18414 |
| 2523 | AAUCUGGA G CUGAUCAC | 7564 | GUGAUCAG GCCGAAAGGCGAGUGAGGUCU UCCAGAUU | 18415 |
| 2540 | UCUAACAU G CACCUGUG | 7567 | CACAGGUG GCCGAAAGGCGAGUGAGGUCU AUGUUAGA | 18416 |
| 2546 | AUGCACCU G UGUGGCUG | 7568 | CAGCCACA GCCGAAAGGCGAGUGAGGUCU AGGUGCAU | 18417 |
| 2548 | GCACCUGU G UGGCUGCG | 7569 | CGCAGCCA GCCGAAAGGCGAGUGAGGUCU ACAGGUGC | 18418 |
| 2551 | CCUGUGUG G CUGCGACU | 7570 | AGUCGCAG GCCGAAAGGCGAGUGAGGUCU CACACAGG | 18419 |
| 2554 | GUGUGGCU G CGACUCUC | 7571 | GAGAGUCG GCCGAAAGGCGAGUGAGGUCU AGCCACAC | 18420 |
| 2568 | CUCUUCUG G CUCCUAUU | 7573 | AAUAGGAG GCCGAAAGGCGAGUGAGGUCU CAGAAGAG | 18421 |
| 2604 | AUGAAAAG G UCUUCUUC | 7576 | GAAGAAGA GCCGAAAGGCGAGUGAGGUCU CUUUUCAU | 18422 |
| 2659 | CAGAUGAA G UUCCUUUG | 7584 | CAAAGGAA GCCGAAAGGCGAGUGAGGUCU UUCAUCUG | 18423 |
| 2673 | UUGGAUGA G CAGUGUGA | 7586 | UCACACUG GCCGAAAGGCGAGUGAGGUCU UCAUCCAA | 18424 |
| 2676 | GAUGAGCA G UGUGAGCG | 7587 | CGCUCACA GCCGAAAGGCGAGUGAGGUCU UGCUCAUC | 18425 |
| 2678 | UGAGCAGU G UGAGCGGC | 7588 | GCCGCUCA GCCGAAAGGCGAGUGAGGUCU ACUGCUCA | 18426 |
| 2682 | CAGUGUGA G CGGCUCCC | 7589 | GGGAGCCG GCCGAAAGGCGAGUGAGGUCU UCACACUG | 18427 |
| 2685 | UGUGAGCG G CUCCCUUA | 7590 | UAAGGGAG GCCGAAAGGCGAGUGAGGUCU CGCUCACA | 18428 |
| 2698 | CUUAUGAU G CCAGCAAG | 7292 | CUUGCUGG GCCGAAAGGCGAGUGAGGUCU AUCAUAAG | 18429 |
| 2702 | UGAUGCCA G CAAGUGGG | 7593 | CCCACUUG GCCGAAAGGCGAGUGAGGUCU UGGCAUCA | 18430 |
| 2706 | GCCAGCAA G UGGGAGUU | 7594 | AACUCCCA GCCGAAAGGCGAGUGAGGUCU UUGCUGGC | 18431 |
| 2712 | AAGUGGGA G UUUGCCCG | 7595 | CGGGCAAA GCCGAAAGGCGAGUGAGGUCU UCCCACUU | 18432 |
| 2716 | GGGAGUUU G CCCGGGAG | 7596 | CUCCCGGG GCCGAAAGGCGAGUGAGGUCU AAACUCCC | 18433 |
| 2738 | UAAACUGG G CAAAUCAC | 7599 | GUGAUUUG GCCGAAAGGCGAGUGAGGUCU CCAGUUUA | 18434 |
| 2758 | GAAGAGGG G CUUUUGGA | 7601 | UCCAAAAG GCCGAAAGGCGAGUGAGGUCU CCCUCUUC | 18435 |
| 2770 | UUGGAAAA G UGGUUCAA | 7602 | UUGAACCA GCCGAAAGGCGAGUGAGGUCU UUUUCCAA | 18436 |
| 2773 | GAAAAGUG G UUCAAGCA | 7603 | UGCUUGAA GCCGAAAGGCGAGUGAGGUCU CACUUUUC | 18437 |
| 2779 | UGGUUCAA G CAUCAGCA | 7604 | UGCUGAUG GCCGAAAGGCGAGUGAGGUCU UUGAACCA | 18438 |
| 2785 | AAGCAUCA G CAUUUGGC | 7605 | GCCAAAUG GCCGAAAGGCGAGUGAGGUCU UGAUGCUU | 18439 |
| 2792 | AGCAUUUG G CAUUAAGA | 7606 | UCUUAAUG GCCGAAAGGCGAGUGAGGUCU CAAAUGCU | 18440 |
| 2811 | UCACCUAC G UGCCGGAC | 7608 | GUCCGGCA GCCGAAAGGCGAGUGAGGUCU GUAGGUGA | 18441 |
| 2813 | ACCUACGU G CCGGACUG | 7609 | CAGUCCGG GCCGAAAGGCGAGUGAGGUCU ACGUAGGU | 18442 |
| 2821 | GCCGGACU G UGGCUGUG | 7611 | CACAGCCA GCCGAAAGGCGAGUGAGGUCU AGUCCGGC | 18443 |
| 2824 | GGACUGUG G CUGUGAAA | 7612 | UUUCACAG GCCGAAAGGCGAGUGAGGUCU CACAGUCC | 18444 |
| 2827 | CUGUGGCU G UGAAAAUG | 7613 | CAUUUUCA GCCGAAAGGCGAGUGAGGUCU AGCCACAG | 18445 |
| 2835 | GUGAAAAU G CUGAAAGA | 7615 | UCUUUCAG GCCGAAAGGCGAGUGAGGUCU AUUUUCAC | 18446 |
| 2848 | AAGAGGGG G CCACGGCC | 7616 | GGCCGUGG GCCGAAAGGCGAGUGAGGUCU CCCCUCUU | 18447 |
| 2854 | GGGCCACG G CCAGCGAG | 7617 | CUCGCUGG GCCGAAAGGCGAGUGAGGUCU CGUGGCCC | 18448 |
| 2858 | CACGGCCA G CGAGUACA | 7618 | UGUACUCG GCCGAAAGGCGAGUGAGGUCU UGGCCGUG | 18449 |
| 2862 | GCCAGCGA G UACAAAGC | 7619 | GCUUUGUA GCCGAAAGGCGAGUGAGGUCU UCGCUGGC | 18450 |
| 2869 | AGUACAAA G CUCUGAUG | 7620 | CAUCAGAG GCCGAAAGGCGAGUGAGGUCU UUUGUACU | 18451 |
| 2883 | AUGACUGA G CUAAAAAU | 7623 | AUUUUUAG GCCGAAAGGCGAGUGAGGUCU UCAGUCAU | 18452 |
| 2906 | CCACAUUG G CCACCAUC | 7626 | GAUGGUGG GCCGAAAGGCGAGUGAGGUCU CAAUGUGG | 18453 |
| 2920 | AUCUGAAC G UGGUUAAC | 7628 | GUUAACCA GCCGAAAGGCGAGUGAGGUCU GUUCAGAU | 18454 |
| 2923 | UGAACGUG G UUAACCUG | 7629 | CAGGUUAA GCCGAAAGGCGAGUGAGGUCU CACGUUCA | 18455 |
| 2931 | GUUAACCU G CUGGGAGC | 7631 | GCUCCCAG GCCGAAAGGCGAGUGAGGUCU AGGUUAAC | 18456 |
| 2938 | UGCUGGGA G CCUGCACC | 7632 | GGUGCAGG GCCGAAAGGCGAGUGAGGUCU UCCCAGCA | 18457 |
| 2942 | GGGAGCCU G CACCAAGC | 7633 | GCUUGGUG GCCGAAAGGCGAGUGAGGUCU AGGCUCCC | 18458 |
| 2949 | UGCACCAA G CAAGGAGG | 7634 | CCUCCUUG GCCGAAAGGCGAGUGAGGUCU UUGGUGCA | 18459 |
| 2958 | CAAGGAGG G CCUCUGAU | 7635 | AUCAGAGG GCCGAAAGGCGAGUGAGGUCU CCUCCUUG | 18460 |
| 2968 | CUCUGAUG G UGAUUGUU | 7637 | AACAAUCA GCCGAAAGGCGAGUGAGGUCU CAUCAGAG | 18461 |
| 2974 | UGGUGAUU G UUGAAUAC | 7639 | GUAUUCAA GCCGAAAGGCGAGUGAGGUCU AAUCACCA | 18462 |
| 2984 | UGAAUACU G CAAAUAUG | 7641 | CAUAUUUG GCCGAAAGGCGAGUGAGGUCU AGUAUUCA | 18463 |
| 3017 | CCUCAAGA G CAAACGUG | 7645 | CACGUUUG GCCGAAAGGCGAGUGAGGUCU UCUUGAGG | 18464 |
| 3023 | GAGCAAAC G UGACUUAU | 7647 | AUAAGUCA GCCGAAAGGCGAGUGAGGUCU GUUUGCUC | 18465 |
| 3049 | ACAAGGAU G CAGCACUA | 7651 | UAGUGCUG GCCGAAAGGCGAGUGAGGUCU AUCCUUGU | 18466 |
| 3052 | AGGAUGCA G CACUACAC | 7652 | GUGUAGUG GCCGAAAGGCGAGUGAGGUCU UGCAUCC8 | 18467 |
| 3066 | CACAUGGA G CCUAAGAA | 7653 | UUCUUAGG GCCGAAAGGCGAGUGAGGUCU UCCAUGUG | 18468 |
| 3087 | AAAAUGGA G CCAGGCCU | 7655 | AGGCCUGG GCCGAAAGGCGAGUGAGGUCU UCCAUUUU | 18469 |
| 3092 | GGAGCCAG G CCUGGAAC | 7656 | GUUCCAGG GCCGAAAGGCGAGUGAGGUCU CUGGCUCC | 18470 |
| 3104 | GGAACAAG G CAAGAAAC | 7658 | GUUUCUUG GCCGAAAGGCGAGUGAGGUCU CUUGUUCC | 18471 |
| 3125 | ACUAGAUA G CGUCACCA | 7662 | UGGUGACG GCCGAAAGGCGAGUGAGGUCU UAUCUAGU | 18472 |
| 3127 | UAGAUAGC G UCACCAGC | 7663 | GCUGGUGA GCCGAAAGGCGAGUGAGGUCU GCUAUCUA | 18473 |
| 3134 | CGUCACCA G CAGCGAAA | 7664 | UUUCGCUG GCCGAAAGGCGAGUGAGGUCU UGGUGACG | 18474 |
| 3137 | CACCAGCA G CGAAAGCU | 7665 | AGCUUUCG GCCGAAAGGCGAGUGAGGUCU UGCUGGUG | 18475 |
| 3143 | CAGCGAAA G CUUUGCGA | 7666 | UCGCAAAG GCCGAAAGGCGAGUGAGGUCU UUUCGCUG | 18476 |
| 3148 | AAAGCUUU G CGAGCUCC | 7667 | GGAGCUCG GCCGAAAGGCGAGUGAGGUCU AAAGCUUU | 18477 |
| 3152 | CUUUGCGA G CUCCGGCU | 7668 | AGCCGGAG GCCGAAAGGCGAGUGAGGUCU UCGCAAAG | 18478 |
| 3158 | GAGCUCCG G CUUUCAGG | 7669 | CCUGAAAG GCCGAAAGGCGAGUGAGGUCU CGGAGCUC | 18479 |
| 3176 | AGAUAAAA G UCUGAGUG | 7671 | CACUCAGA GCCGAAAGGCGAGUGAGGUCU UUUUAUCU | 18480 |
| 3182 | AAGUCUGA G UGAUGUUG | 7672 | CAACAUCA GCCGAAAGGCGAGUGAGGUCU UCAGACUU | 18481 |
| 3187 | UGAGUGAU G UUGAGGAA | 7674 | UUCCUCAA GCCGAAAGGCGAGUGAGGUCU AUCACUCA | 18482 |
| 3212 | UUCUGACG G UUUCUACA | 7677 | UGUAGAAA GCCGAAAGGCGAGUGAGGUCU CGUCAGAA | 18483 |
| 3225 | UACAAGGA G CCCAUCAC | 7678 | GUGAUGGG GCCGAAAGGCGAGUGAGGUCU UCCUUGUA | 18484 |
| 3257 | UUCUUACA G UUUUCAAG | 7681 | CUUGAAAA GCCGAAAGGCGAGUGAGGUCU UGUAAGAA | 18485 |
| 3265 | GUUUUCAA G UGGCCAGA | 7682 | UCUGGCCA GCCGAAAGGCGAGUGAGGUCU UUGAAAAC | 18486 |
| 3268 | UUCAAGUG G CCAGAGGC | 7683 | GCCUCUGG GCCGAAAGGCGAGUGAGGUCU CACUUGAA | 18487 |
| 3275 | GGCCAGAG G CAUGGAGU | 7684 | ACUCCAUG GCCGAAAGGCGAGUGAGGUCU CUCUGGCC | 18488 |
| 3282 | GGCAUGGA G UUCCUGUC | 7685 | GACAGGAA GCCGAAAGGCGAGUGAGGUCU UCCAUGCC | 18489 |
| 3288 | GAGUUCCU G UCUUCCAG | 7686 | CUGGAAGA GCCGAAAGGCGAGUGAGGUCU AGGAACUC | 18490 |
| 3300 | UCCAGAAA G UGCAUUCA | 7687 | UGAAUGCA GCCGAAAGGCGAGUGAGGUCU UUUCUGGA | 18491 |
| 3302 | CAGAAAGU G CAUUCAUC | 7688 | GAUGAAUG GCCGAAAGGCGAGUGAGGUCU ACUUUCUG | 18492 |
| 3319 | GGGACCUG G CAGCGAGA | 7690 | UCUCGCUG GCCGAAAGGCGAGUGAGGUCU CAGGUCCC | 18493 |
| 3322 | ACCUGGCA G CGAGAAAC | 7691 | GUUUCUCG GCCGAAAGGCGAGUGAGGUCU UGCCAGGU | 18494 |
| 3352 | AGAACAAC G UGGUGAAG | 7695 | CUUCACCA GCCGAAAGGCGAGUGAGGUCU GUUGUUCU | 18495 |
| 3355 | ACAACGUG G UGAAGAUU | 7696 | AAUCUUCA GCCGAAAGGCGAGUGAGGUCU CACGUUGU | 18496 |
| 3365 | GAAGAUUU G UGAUUUUG | 7698 | CAAAAUCA GCCGAAAGGCGAGUGAGGUCU AAAUCUUC | 18497 |
| 3374 | UGAUUUUG G CCUUGCCC | 7700 | GGGCAAGG GCCGAAAGGCGAGUGAGGUCU CAAAAUCA | 18498 |
| 3379 | UUGGCCUU G CCCGGGAU | 7701 | AUCCCGGG GCCGAAAGGCGAGUGAGGUCU AAGGCCAA | 18499 |
| 3409 | CCGAUUAU G UGAGAAAA | 7705 | UUUUCUCA GCCGAAAGGCGAGUGAGGUCU AUAAUCGG | 18500 |
| 3448 | AAUGGAUG G CUCCCGAA | 7710 | UUCGGGAG GCCGAAAGGCGAGUGAGGUCU CAUCCAUU | 18501 |
| 3479 | AAUCUACA G CACCAAGA | 7714 | UCUUGGUG GCCGAAAGGCGAGUGAGGUCU UGUAGAUU | 18502 |
| 3488 | CACCAAGA G CGACGUGU | 7715 | ACACGUCG GCCGAAAGGCGAGUGAGGUCU UCUUGGUG | 18503 |
| 3493 | AGAGCGAC G UGUGGUCU | 7717 | AGACCACA GCCGAAAGGCGAGUGAGGUCU GUCGCUCU | 18504 |
| 3495 | AGCGACGU G UGGUCUUA | 7718 | UAAGACCA GCCGAAAGGCGAGUGAGGUCU ACGUCGCU | 18505 |
| 3498 | GACGUGUG G UCUUACGG | 7719 | CCGUAAGA GCCGAAAGGCGAGUGAGGUCU CACACGUC | 18506 |
| 3508 | CUUACGGA G UAUUGCUG | 7720 | CAGCAAUA GCCGAAAGGCGAGUGAGGUCU UCCGUAAG | 18507 |
| 3513 | UUAGGUGG G UCUCCAUA | 7721 | UAUGGAGA GCCGAAAGGCGAGUGAGGUCU CCACCUAA | 18508 |
| 3516 | ACCCAGGA G UACAAAUG | 7722 | CAUUUGUA GCCGAAAGGCGAGUGAGGUCU UCCUGGGU | 18509 |
| 3536 | GGACUUUU G CAGUCGCC | 7724 | GGCGACUG GCCGAAAGGCGAGUGAGGUCU AAAAGUCC | 18510 |
| 3540 | UUAGGUGG G UCUCCAUA | 7725 | UAUGGAGA GCCGAAAGGCGAGUGAGGUCU CCACCUAA | 18511 |
| 3556 | ACCCAGGA G UACAAAUG | 7726 | CAUUUGUA GCCGAAAGGCGAGUGAGGUCU UCCUGGGU | 18512 |
| 3578 | GGACUUUU G CAGUCGCC | 7730 | GGCGACUG GCCGAAAGGCGAGUGAGGUCU AAAAGUCC | 18513 |
| 3581 | CUUUUGCA G UCGCCUGA | 7731 | UCAGGCGA GCCGAAAGGCGAGUGAGGUCU UGCAAAAG | 18514 |
| 3584 | UUGCAGUC G CCUGAGGG | 7732 | CCCUCAGG GCCGAAAGGCGAGUGAGGUCU GACUGCAA | 18515 |
| 3596 | GAGGGAAG G CAUGAGGA | 7733 | UCCUCAUG GCCGAAAGGCGAGUGAGGUCU CUUCCCUC | 18516 |
| 3610 | GGAUGAGA G CUCCUGAG | 7735 | CUCAGGAG GCCGAAAGGCGAGUGAGGUCU UCUCAUCC | 18517 |
| 3618 | GCUCCUGA G UACUCUAC | 7736 | GUAGAGUA GCCGAAAGGCGAGUGAGGUCU UCAGGAGC | 18518 |
| 3648 | CAGAUCAU G CUGGACUG | 7739 | CAGUCCAG GCCGAAAGGCGAGUGAGGUCU AUGAUCUG | 18519 |
| 3656 | GCUGGACU G CUGGCACA | 7741 | UGUGCCAG GCCGAAAGGCGAGUGAGGUCU AGUCCAGC | 18520 |
| 3660 | GACUGCUG G CACAGAGA | 7742 | UCUCUGUG GCCGAAAGGCGAGUGAGGUCU CAGCAGUC | 18521 |
| 3681 | AAAGAAAG G CCAAGAUU | 7744 | AAUCUUGG GCCGAAAGGCGAGUGAGGUCU CUUUCUUU | 18522 |
| 3691 | CAAGAUUU G CAGAACUU | 7746 | AAGUUCUG GCCGAAAGGCGAGUGAGGUCU AAAUCUUG | 18523 |
| 3700 | CAGAACUU G UGGAAAAA | 7748 | UUUUUCCA GCCGAAAGGCGAGUGAGGUCU AAGUUCUG | 18524 |
| 3713 | AAAACUAG G UGAUUUGC | 7750 | GCAAAUCA GCCGAAAGGCGAGUGAGGUCU CUAGUUUU | 18525 |
| 3720 | GGUGAUUU G CUUCAAGC | 7752 | GCUUGAAG GCCGAAAGGCGAGUGAGGUCU AAAUCACC | 18526 |
| 3727 | UGCUUCAA G CAAAUGUA | 7753 | UACAUUUG GCCGAAAGGCGAGUGAGGUCU UUGAAGCA | 18527 |
| 3733 | AAGCAAAU G UACAACAG | 7755 | CUGUUGUA GCCGAAAGGCGAGUGAGGUCU AUUUGCUU | 18528 |
| 3746 | ACAGGAUG G UAAAGACU | 7758 | AGUCUUUA GCCGAAAGGCGAGUGAGGUCU CAUCCUGU | 18529 |
| 3769 | CAAUCAAU G CCAUACUG | 7762 | CAGUAUGG GCCGAAAGGCGAGUGAGGUCU AUUGAUUG | 18530 |
| 3788 | AGGAAAUA G UGGGUUUA | 7765 | UAAACCCA GCCGAAAGGCGAGUGAGGUCU UAUUUCCU | 18531 |
| 3792 | AAUAGUGG G UUUACAUA | 7766 | UAUGUAAA GCCGAAAGGCGAGUGAGGUCU CCACUAUU | 18532 |
| 3811 | CAACUCCU G CCUUCUCU | 7768 | AGAGAAGG GCCGAAAGGCGAGUGAGGUCU AGGAGUUG | 18533 |
| 3839 | CAAGGAAA G UAUUUCAG | 7770 | CUGAAAUA GCCGAAAGGCGAGUGAGGUCU UUUCCUUG | 18534 |
| 3847 | GUAUUUCA G CUCCGAAG | 7771 | CUUCGGAG GCCGAAAGGCGAGUGAGGUCU UGAAAUAC | 18535 |
| 3855 | GCUCCGAA G UUUAAUUC | 7772 | GAAUUAAA GCCGAAAGGCGAGUGAGGUCU UUCGGAGC | 18536 |
| 3869 | UUCAGGAA G CUCUGAUG | 7774 | CAUCAGAG GCCGAAAGGCGAGUGAGGUCU UUCCUGAA | 18537 |
| 3880 | CUGAUGAU G UCAGAUAU | 7777 | AUAUCUGA GCCGAAAGGCGAGUGAGGUCU AUCAUCAG | 18538 |
| 3889 | UCAGAUAU G UAAAUGCU | 7779 | AGCAUUUA GCCGAAAGGCGAGUGAGGUCU UAUUCUGA | 18539 |
| 3895 | AUGUAAAU G CUUUCAAG | 7781 | CUUGAAAG GCCGAAAGGCGAGUGAGGUCU AUUUACAU | 18540 |
| 3903 | GCUUUCAA G UUCAUGAG | 7782 | CUCAUGAA GCCGAAAGGCGAGUGAGGUCU UUGAAAGC | 18541 |
| 3911 | GUUCAUGA G CCUGGAAA | 7783 | UUUCCAGG GCCGAAAGGCGAGUGAGGUCU UCAUGAAC | 18542 |
| 3952 | UACCGAAU G CCACCUCC | 7788 | GGAGGUGG GCCGAAAGGCGAGUGAGGUCU AUUCGGUA | 18543 |
| 3963 | ACCUCCAU G UUUGAUGA | 7789 | UCAUCAAA GCCGAAAGGCGAGUGAGGUCU AUGGAGGU | 18544 |
| 3980 | CUACCAGG G CGACAGCA | 7792 | UGCUGUCG GCCGAAAGGCGAGUGAGGUCU AUGGAGGU | 18545 |
| 3986 | GGGCGACA G CAGCACUC | 7794 | GAGUGCUG GCCGAAAGGCGAGUGAGGUCU UGUCGCCC | 18546 |
| 3989 | CGACAGCA G CACUCUGU | 7795 | ACAGAGUG GCCGAAAGGCGAGUGAGGUCU UGCUGUCG | 18547 |
| 3996 | AGCACUCU G UUGGCCUC | 7796 | GAGGCCAA GCCGAAAGGCGAGUGAGGUCU AGAGUGCU | 18548 |
| 4000 | CUCUGUUG G CCUCUCCC | 7797 | GGGAGAGG GCCGAAAGGCGAGUGAGGUCU CAACAGAG | 18549 |
| 4011 | UCUCCCAU G CUGAAGCG | 7798 | CGCUUCAG GCCGAAAGGCGAGUGAGGUCU AUGGGAGA | 18550 |
| 4017 | AUGCUGAA G CGCUUCAC | 7799 | GUGAAGCG GCCGAAAGGCGAGUGAGGUCU UUCAGCAU | 18551 |
| 4019 | GCUGAAGC G CUUCACCU | 7800 | AGGUGAAG GCCGAAAGGCGAGUGAGGUCU GCUUCAGC | 18552 |
| 4037 | GACUGACA G CAAACCCA | 7803 | UGGGUUUG GCCGAAAGGCGAGUGAGGUCU UGUCAGUC | 18553 |
| 4048 | AACCCAAG G CCUCGCUC | 7805 | GAGCGAGG GCCGAAAGGCGAGUGAGGUCU CUUGGGUU | 18554 |
| 4053 | AAGGCCUC G CUCAAGAU | 7806 | AUCUUGAG GCCGAAAGGCGAGUGAGGUCU GAGGCCUU | 18555 |
| 4072 | ACUUGAGA G UAACCAGU | 7809 | ACUGGUUA GCCGAAAGGCGAGUGAGGUCU UCUCAAGU | 18556 |
| 4079 | AGUAACCA G UAAAAGUA | 7811 | UACUUUUA GCCGAAAGGCGAGUGAGGUCU UGGUUACU | 18557 |
| 4085 | CAGUAAAA G UAAGGAGU | 7812 | ACUCCUUA GCCGAAAGGCGAGUGAGGUCU UUUUACUG | 18558 |
| 4092 | AGUAAGGA G UCGGGGCU | 7813 | AGCCCCGA GCCGAAAGGCGAGUGAGGUCU UCCUUACU | 18559 |
| 4098 | GAGUCGGG G CUGUCUGA | 7814 | UCAGACAG GCCGAAAGGCGAGUGAGGUCU CCCGACUC | 18560 |
| 4101 | UCGGGGCU G UCUGAUGU | 7815 | ACAUCAGA GCCGAAAGGCGAGUGAGGUCU AGCCCCGA | 18561 |
| 4108 | UGUCUGAU G UCAGCAGG | 7817 | CCUGCUGA GCCGAAAGGCGAGUGAGGUCU AUCAGACA | 18562 |
| 4112 | UGAUGUCA G CAGGCCCA | 7818 | UGGGCCUG GCCGAAAGGCGAGUGAGGUCU UGACAUCA | 18563 |
| 4116 | GUCAGCAG G CCCAGUUU | 7819 | AAACUGGG GCCGAAAGGCGAGUGAGGUCU CUGCUGAC | 18564 |
| 4121 | CAGGCCCA G UUUCUGCC | 7820 | GGCAGAAA GCCGAAAGGCGAGUGAGGUCU UGGGCCUG | 18565 |
| 4127 | CAGUUUCU G CCAUUCCA | 7821 | UGGAAUGG GCCGAAAGGCGAGUGAGGUCU AGAAACUG | 18566 |
| 4136 | CCAUUCCA G CUGUGGGC | 7822 | GCCCACAG GCCGAAAGGCGAGUGAGGUCU UGGAAUGG | 18567 |
| 4139 | UUCCAGCU G UGGGCACG | 7823 | CGUGCCCA GCCGAAAGGCGAGUGAGGUCU AGCUGGAA | 18568 |
| 4143 | AGCUGUGG G CACGUCAG | 7824 | CUGACGUG GCCGAAAGGCGAGUGAGGUCU CCACAGCU | 18569 |
| 4147 | GUGGGCAC G UCAGCGAA | 7825 | UUCGCUGA GCCGAAAGGCGAGUGAGGUCU GUGCCCAC | 18570 |
| 4151 | GCACGUCA G CGAAGGCA | 7826 | UGCCUUCG GCCGAAAGGCGAGUGAGGUCU UGACGUGC | 18571 |
| 4157 | CAGCGAAG G CAAGCGCA | 7827 | UGCGCUUG GCCGAAAGGCGAGUGAGGUCU CUUCGCUG | 18572 |
| 4161 | GAAGGCAA G CGCAGGUU | 7828 | AACCUGCG GCCGAAAGGCGAGUGAGGUCU UUGCCUUC | 18573 |
| 4163 | AGGCAAGC G CAGGUUCA | 7829 | UGAACCUG GCCGAAAGGCGAGUGAGGUCU GCUUGCCU | 18574 |
| 4167 | AAGCGCAG G UUCACCUA | 7830 | UAGGUGAA GCCGAAAGGCGAGUGAGGUCU CUGCGCUU | 18575 |
| 4183 | ACGACCAC G CUGAGCUG | 7832 | CAGCUCAG GCCGAAAGGCGAGUGAGGUCU GUGGUCGU | 18576 |
| 4188 | CACGCUGA G CUGGAAAG | 7833 | CUUUCCAG GCCGAAAGGCGAGUGAGGUCU UCAGCGUG | 18577 |
| 4204 | GGAAAAUC G CGUGCUGC | 7835 | GCAGCACG GCCGAAAGGCGAGUGAGGUCU GAUUUUCC | 18578 |
| 4206 | AAAAUCGC G UGCUGCUC | 7836 | GAGCAGCA GCCGAAAGGCGAGUGAGGUCU GCGAUUUU | 18579 |
| 4208 | AAUCGCGU G CUGCUCCC | 7837 | GGGAGCAG GCCGAAAGGCGAGUGAGGUCU ACGCGAUU | 18580 |
| 4211 | CGCGUGCU G CUCCCCGC | 7838 | GCGGGGAG GCCGAAAGGCGAGUGAGGUCU AGCACGCG | 18581 |
| 4218 | UGCUCCCC G CCCCCAGA | 7839 | UCUGGGGG GCCGAAAGGCGAGUGAGGUCU GGGGAGCA | 18582 |
| 4237 | ACAACUCG G UGGUCCUG | 7842 | CAGGACCA GCCGAAAGGCGAGUGAGGUCU CGAGUUGU | 18583 |
| 4240 | ACUCGGUG G UCCUGUAC | 7843 | GUACAGGA GCCGAAAGGCGAGUGAGGUCU CACCGAGU | 18584 |
| 4245 | GUGGUCCU G UACUCCAC | 7844 | GUGGAGUA GCCGAAAGGCGAGUGAGGUCU AGGACCAC | 18585 |
| 4268 | CAUCUAGA G UUUGACAC | 7845 | GUGUCAAA GCCGAAAGGCGAGUGAGGUCU UCUAGAUG | 18586 |
| 4280 | GACACGAA G CCUUAUUU | 7847 | AAAUAAGG GCCGAAAGGCGAGUGAGGUCU UUCGUGUC | 18587 |
| 4295 | UUCUAGAA G CACAUGUG | 7848 | CACAUGUG GCCGAAAGGCGAGUGAGGUCU UUCUAGAA | 18588 |
| 4301 | AAGCACAU G UGUAUUUA | 7849 | UAAAUACA GCCGAAAGGCGAGUGAGGUCU AUGUGCUU | 18589 |
| 4303 | GCACAUGU G UAUUUAUA | 7850 | UAUAAAUA GCCGAAAGGCGAGUGAGGUCU ACAUGUGC | 18590 |
| 4326 | GGAAACUA G CUUUUGCC | 7852 | GGCAAAAG GCCGAAAGGCGAGUGAGGUCU UAGUUUCC | 18591 |
| 4332 | UAGCUUUU G CCAGUAUU | 7853 | AAUACUGG GCCGAAAGGCGAGUGAGGUCU AAAAGCUA | 18592 |
| 4336 | UUUUGCCA G UAUUAUGC | 7854 | GCAUAAUA GCCGAAAGGCGAGUGAGGUCU UGGCAAAA | 18593 |
| 4343 | AGUAUUAU G CAUAUAUA | 7855 | UAUAUAUG GCCGAAAGGCGAGUGAGGUCU AUAAUACU | 18594 |
| 4353 | AUAUAUAA G UUUACACC | 7856 | GGUGUAAA GCCGAAAGGCGAGUGAGGUCU UUAUAUAU | 18595 |
| 4379 | CCAUGGGA G CCAGCUGC | 7857 | GCAGCUGG GCCGAAAGGCGAGUGAGGUCU UCCCAUGG | 18596 |
| 4383 | GGGAGCCA G CUGCUUUU | 7858 | AAAAGCAG GCCGAAAGGCGAGUGAGGUCU UGGCUCCC | 18597 |
| 4386 | AGCCAGCU G CUUUUUGU | 7859 | ACAAAAAG GCCGAAAGGCGAGUGAGGUCU AGCUGGCU | 18598 |
| 4393 | UGCUUUUU G UGAUUUUU | 7860 | AAAAAUCA GCCGAAAGGCGAGUGAGGUCU AAAAAGCA | 18599 |
| 4408 | UUUUAAUA G UGCUUUUU | 7863 | AAAAAGCA GCCGAAAGGCGAGUGAGGUCU UAUUAAAA | 18600 |
| 4410 | UUAAUAGU G CUUUUUUU | 7864 | AAAAAAAG GCCGAAAGGCGAGUGAGGUCU ACUAUUAA | 18601 |
| 4436 | ACAAGAAU G UAACUCCA | 7868 | UGGAGUUA GCCGAAAGGCGAGUGAGGUCU AUUCUUGU | 18602 |
| 4457 | GAGAAAUA G UGACAAGU | 7872 | ACUUGUCA GCCGAAAGGCGAGUGAGGUCU UAUUUCUC | 18603 |
| 4464 | AGUGACAA G UGAAGAAC | 7874 | GUUCUUCA GCCGAAAGGCGAGUGAGGUCU UUGUCACU | 18604 |
| 4479 | ACACUACU G CUAAAUCC | 7876 | GGAUUUAG GCCGAAAGGCGAGUGAGGUCU AGUAGUGU | 18605 |
| 4492 | AUCCUCAU G UUACUCAG | 7878 | CUGAGUAA GCCGAAAGGCGAGUGAGGUCU AUGAGGAU | 18606 |
| 4500 | GUUACUCA G UGUUAGAG | 7879 | CUCUAACA GCCGAAAGGCGAGUGAGGUCU UGAGUAAC | 18607 |
| 4502 | UACUCAGU G UUAGAGAA | 7880 | UUCUCUAA GCCGAAAGGCGAGUGAGGUCU ACUGAGUA | 18608 |
| 4538 | ACUUCCCU G CUCCAACC | 7885 | GGUUGGAG GCCGAAAGGCGAGUGAGGUCU AGGGAAGU | 18609 |
| 4550 | CAACCCCC G CCACCUCA | 7887 | UGAGGUGG GCCGAAAGGCGAGUGAGGUCU GGGGGUUG | 18610 |
| 4561 | ACCUCAGG G CACGCAGG | 7888 | CCUGCGUG GCCGAAAGGCGAGUGAGGUCU CCUGAGGU | 18611 |
| 4565 | CAGGGCAC G CAGGACCA | 7889 | UGGUCCUG GCCGAAAGGCGAGUGAGGUCU GUGCCCUG | 18612 |
| 4574 | CAGGACCA G UUUGAUUG | 7891 | CAAUCAAA GCCGAAAGGCGAGUGAGGUCU UGGUCCUG | 18613 |
| 4587 | AUUGAGGA G CUGCACUG | 7893 | CAGUGCAG GCCGAAAGGCGAGUGAGGUCU UCCUCAAU | 18614 |
| 4590 | GAGGAGCU G CACUGAUC | 7894 | GAUCAGUG GCCGAAAGGCGAGUGAGGUCU AGCUCCUC | 18615 |
| 4606 | CACCCAAU G CAUCACGU | 7897 | ACGUGAUG GCCGAAAGGCGAGUGAGGUCU AUUGGGUG | 18616 |
| 4613 | UGCAUCAC G UACCCCAC | 7898 | GUGGGGUA GCCGAAAGGCGAGUGAGGUCU GUGAUGCA | 18617 |
| 4625 | CCCACUGG G CCAGCCCU | 7899 | AGGGCUGG GCCGAAAGGCGAGUGAGGUCU CCAGUGGG | 18618 |
| 4629 | CUGGGCCA G CCCUGCAG | 7900 | CUGCAGGG GCCGAAAGGCGAGUGAGGUCU UGGCCCAG | 18619 |
| 4634 | CCAGCCCU G CAGCCCAA | 7901 | UUGGGCUG GCCGAAAGGCGAGUGAGGUCU AGGGCUGG | 18620 |
| 4637 | GCCCUGCA G CCCAAAAC | 7902 | GUUUUGGG GCCGAAAGGCGAGUGAGGUCU UGCAGGGC | 18621 |
| 4651 | AACCCAGG G CAACAAGC | 7904 | GCUUGUUG GCCGAAAGGCGAGUGAGGUCU CCUGGGUU | 18622 |
| 4658 | GGCAACAA G CCCGUUAG | 7906 | CUAACGGG GCCGAAAGGCGAGUGAGGUCU UUGUUGCC | 18623 |
| 4662 | ACAAGCCC G UUAGCCCC | 7907 | GGGGCUAA GCCGAAAGGCGAGUGAGGUCU GGGCUUGU | 18624 |
| 4666 | GCCCGUUA G CCCCAGGG | 7908 | CCCUGGGG GCCGAAAGGCGAGUGAGGUCU UAACGGGC | 18625 |
| 4683 | GAUCACUG G CUGGCCUG | 7910 | CAGGCCAG GCCGAAAGGCGAGUGAGGUCU CAGUGAUC | 18626 |
| 4687 | ACUGGCUG G CCUGAGCA | 7911 | UGCUCAGG GCCGAAAGGCGAGUGAGGUCU CAGCCAGU | 18627 |
| 4693 | UGGCCUGA G CAACAUCU | 7912 | AGAUGUUG GCCGAAAGGCGAGUGAGGUCU UCAGGCCA | 18628 |
| 4707 | UCUCGGGA G UCCUCUAG | 7914 | CUAGAGGA GCCGAAAGGCGAGUGAGGUCU UCCCGAGA | 18629 |
| 4715 | GUCCUCUA G CAGGCCUA | 7915 | UAGGCCUG GCCGAAAGGCGAGUGAGGUCU UAGAGGAC | 18630 |
| 4719 | UCUAGCAG G CCUAAGAC | 7916 | GUCUUAGG GCCGAAAGGCGAGUGAGGUCU CUGCUAGA | 18631 |
| 4730 | UAAGACAU G UGAGGAGG | 7918 | CCUCCUCA GCCGAAAGGCGAGUGAGGUCU AUGUCUUA | 18632 |
| 4752 | GAAAAAAA G CAAAAAGC | 7919 | GCUUUUUG GCCGAAAGGCGAGUGAGGUCU UUUUUUUC | 18633 |
| 4759 | AGCAAAAA G CAAGGGAG | 7920 | CUCCCUUG GCCGAAAGGCGAGUGAGGUCU UUUUUGCU | 18634 |
| 4788 | GGGAGAAG G CAUGAGAA | 7922 | UUCUCAUG GCCGAAAGGCGAGUGAGGUCU CUUCUCCC | 18635 |
| 4809 | UUUGAGAC G CACCAUGU | 7925 | ACAUGGUG GCCGAAAGGCGAGUGAGGUCU GUCUCAAA | 18636 |
| 4816 | CGCACCAU G UGGGCACG | 7926 | CGUGCCCA GCCGAAAGGCGAGUGAGGUCU AUGGUGCG | 18637 |
| 4820 | CCAUGUGG G CACGGAGG | 7927 | CCUCCGUG GCCGAAAGGCGAGUGAGGUCU CCACAUGG | 18638 |
| 4837 | GGGACGGG G CUCAGCAA | 7929 | UUGCUGAG GCCGAAAGGCGAGUGAGGUCU CCCGUCCC | 18639 |
| 4842 | GGGGCUCA G CAAUGCCA | 7930 | UGGCAUUG GCCGAAAGGCGAGUGAGGUCU UGACCCCC | 18640 |
| 4847 | UCAGCAUU G CCAUUUCA | 7932 | UGAAAUGG GCCGAAAGGCGAGUGAGGUCU CACUGAAA | 18641 |
| 4856 | CCAUUUCA G UGGCUUCC | 7933 | GGAAGCCA GCCGAAAGGCGAGUGAGGUCU UGAAAUGG | 18642 |
| 4859 | UUUCAGUG G CUUCCCAG | 7934 | CUGGGAAG GCCGAAAGGCGAGUGAGGUCU CACUGAAA | 18643 |
| 4867 | GCUUCCCA G CUCUGACC | 7935 | GGUCAGAG GCCGAAAGGCGAGUGAGGUCU UGGGAAGC | 18644 |
| 4891 | AUUUGAGG G CCCAGCCA | 7937 | UGGCUGGG GCCGAAAGGCGAGUGAGGUCU CCUCAAAU | 18645 |
| 4896 | AGGGCCCA G CCAGGAGC | 7938 | GCUCCUGG GCCGAAAGGCGAGUGAGGUCU UGGGCCCU | 18646 |
| 4903 | AGCCAGGA G CAGAUGGA | 7939 | UCCAUCUG GCCGAAAGGCGAGUGAGGUCU UCCUGGCU | 18647 |
| 4914 | GAUGGACA G CGAUGAGG | 7942 | CCUCAUCG GCCGAAAGGCGAGUGAGGUCU UGUCCAUC | 18648 |
| 4946 | UCUGGGAG G CAAGAAAA | 7946 | UUUUCUUG GCCGAAAGGCGAGUGAGGUCU CUCCCAGA | 18649 |
| 4981 | GAACUAAA G CAAAUUUU | 7950 | AAAAUUUG GCCGAAAGGCGAGUGAGGUCU UUUAGUUC | 18650 |
| 5008 | CUAUGGAA G UGGUUCUA | 7953 | UAGAACCA GCCGAAAGGCGAGUGAGGUCU UUCCAUAG | 18651 |
| 5011 | UGGAAGUG G UUCUAUGU | 7954 | ACAUAGAA GCCGAAAGGCGAGUGAGGUCU CACUUCCA | 18652 |
| 5018 | GGUUCUAU G UCCAUUCU | 7955 | AAAACAUG GCCGAAAGGCGAGUGAGGUCU CACGAAUG | 18653 |
| 5032 | UCUCAUUC G UGGCAUGU | 7956 | AAUCAAAA GCCGAAAGGCGAGUGAGGUCU AUGCCACG | 18654 |
| 5035 | CAUUCGUG G CAUGUUUU | 7957 | AAAACAUG GCCGAAAGGCGAGUGAGGUCU AAAUCAAA | 18655 |
| 5039 | CGUGGCAU G UUUUGAUU | 7958 | AAUCAAAA GCCGAAAGGCGAGUGAGGUCU UACAAAUC | 18656 |
| 5049 | UUUGAUUU G UAGCACUG | 7960 | CAGUGCUA GCCGAAAGGCGAGUGAGGUCU AAAUCAAA | 18657 |
| 5052 | GAUUUGUA G CACUGAGG | 7961 | CCUCAGUG GCCGAAAGGCGAGUGAGGUCU UACAAAUC | 18658 |
| 5061 | CACUGAGG G UGGCACUC | 7962 | GAGUGCCA GCCGAAAGGCGAGUGAGGUCU CCUCAGUG | 18659 |
| 5064 | UGAGGGUG G CACUGAGG | 7963 | GUUGAGUG GCCGAAAGGCGAGUGAGGUCU CACCCUCA | 18660 |
| 5078 | AACUCUGA G CCCAUACU | 7965 | AGUAUGGG GCCGAAAGGCGAGUGAGGUCU UCAGAGUU | 18661 |
| 5091 | UACUUUUG G CUCCUCUA | 7966 | UAGAGGAG GCCGAAAGGCGAGUGAGGUCU CAAAAGUA | 18662 |
| 5100 | CUCCUCUA G UAAGAUGC | 7967 | GCAUCUUA GCCGAAAGGCGAGUGAGGUCU UAGAGGAG | 18663 |
| 5107 | AGUAAGAU G CACUGAAA | 7969 | UUUCAGUG GCCGAAAGGCGAGUGAGGUCU AUCUUACU | 18664 |
| 5121 | AAAACUUA G CCAGAGUU | 7971 | AACUCUGG GCCGAAAGGCGAGUGAGGUCU UAAGUUUU | 18665 |
| 5127 | UAGCCAGA G UUAGGUUG | 7972 | CAACCUAA GCCGAAAGGCGAGUGAGGUCU UCUGGCUA | 18666 |
| 5132 | AGAGUUAG G UUGUCUCC | 7973 | GGAGACAA GCCGAAAGGCGAGUGAGGUCU CUAACUCU | 18667 |
| 5135 | GUUAGGUU G UCUCCAGG | 7974 | CCUGGAGA GCCGAAAGGCGAGUGAGGUCU AACCUAAC | 18668 |
| 5143 | GUCUCCAG G CCAUGAUG | 7975 | CAUCAUGG GCCGAAAGGCGAGUGAGGUCU CUGGAGAC | 18669 |
| 5152 | CCAUGAUG G CCUUACAC | 7977 | GUGUAAGG GCCGAAAGGCGAGUGAGGUCU CAUCAUGG | 18670 |
| 5168 | CUGAAAAU G UCACAUUC | 7979 | GAAUGUGA GCCGAAAGGCGAGUGAGGUCU AUUUUCAG | 18671 |
| 5185 | UAUUUUGG G UAUUAAUA | 7980 | UAUUAAUA GCCGAAAGGCGAGUGAGGUCU CCAAAAUA | 18672 |
| 5198 | AAUAUAUA G UCCAGACA | 7982 | UGUCUGGA GCCGAAAGGCGAGUGAGGUCU UAUAUAUU | 18673 |
| 5224 | AUUUCUUG G UAUUAUUC | 7986 | GAAUAAUA GCCGAAAGGCGAGUGAGGUCU CAAGAAAU | 18674 |
| 5234 | AUUAUUCU G UUUUGCAC | 7987 | GUGCAAAA GCCGAAAGGCGAGUGAGGUCU AGAAUAAU | 18675 |
| 5239 | UCUGUUUU G CACAGUUA | 7988 | UAACUGUG GCCGAAAGGCGAGUGAGGUCU AAAACAGA | 18676 |
| 5244 | UUUGCACA G UUAGUUGU | 7989 | ACAACUAA GCCGAAAGGCGAGUGAGGUCU UGUGCAAA | 18677 |
| 5248 | CACAGUUA G UUGUGAAA | 7990 | UUUCACAA GCCGAAAGGCGAGUGAGGUCU UAACUGUG | 18678 |
| 5251 | AGUUAGUU G UGAAAGAA | 7991 | UUCUUUCA GCCGAAAGGCGAGUGAGGUCU AACUAACU | 18679 |
| 5261 | GAAAGAAA G CUGAGAAG | 7992 | CUUCUCAG GCCGAAAGGCGAGUGAGGUCU UUUCUUUC | 18680 |
| 5279 | AUGAAAAU G CAGUCCUG | 7995 | CAGGACUG GCCGAAAGGCGAGUGAGGUCU AUUUUCAU | 18681 |
| 5282 | AAAAUGCA G UCCUGAGG | 7996 | CCUCAGGA GCCGAAAGGCGAGUGAGGUCU UGCAUUUU | 18682 |
| 5294 | UGAGGAGA G UUUUCUCC | 7997 | GGAGAAAA GCCGAAAGGCGAGUGAGGUCU UCUCCUCA | 18683 |
| 5317 | AAACGAGG G CUGAUGGA | 7999 | UCCAUCAG GCCGAAAGGCGAGUGAGGUCU CCUCGUUU | 18684 |
| 5334 | GGAAAAAG G UCAAUAAG | 8001 | UAUAGAGA GCCGAAAGGCGAGUGAGGUCU CUUUUUCC | 18685 |
| 5343 | UCAAUAAG G UCAAGGGA | 8003 | GGUCCCAA GCCGAAAGGCGAGUGAGGUCU CUUAUUGA | 18686 |
| 5359 | AAGACCCC G UCUCUAUA | 8005 | GUGACUGA GCCGAAAGGCGAGUGAGGUCU GGGGUCUU | 18687 |
| 5393 | CCAACACA G UUGGGACC | 8010 | AAACGUGA GCCGAAAGGCGAGUGAGGUCU UGACUUCC | 18688 |
| 5415 | CACAGGAA G UCAGUCAC | 8013 | AAAGGAAA GCCGAAAGGCGAGUGAGGUCU UUCCUGUG | 18689 |
| 5419 | GGAAGUCA G UCACGUUU | 8014 | AAACGUGA GCCGAAAGGCGAGUGAGGUCU UGACUUCC | 18690 |
| 5424 | UCAGUCAC G UUUCCUUU | 8015 | CUCUUCCA GCCGAAAGGCGAGUGAGGUCU GUGACUGA | 18691 |
| 5477 | GAAAGGAU G UGGAAGAG | 8020 | AGCUAAUG GCCGAAAGGCGAGUGAGGUCU AUCCUUUC | 18692 |
| 5485 | GUGGAAGA G CAUUAGCU | 8021 | UGCGCCAG GCCGAAAGGCGAGUGAGGUCU UAAUGCUC | 18693 |
| 5491 | GAGCAUUA G CUGGCGCA | 8022 | AAUAUGCG GCCGAAAGGCGAGUGAGGUCU CAGCUAAU | 18694 |
| 5495 | AUUAGCUG G CGCAUAUU | 8023 | AAUAUGCG GCCGAAAGGCGAGUGAGGUCU GCCAGCUA | 18695 |
| 5497 | UAGCUGGC G CAUAUUAA | 8024 | UUAAUAUG GCCGAAAGGCGAGUGAGGUCU UUAAUAUG | 18696 |
| 5506 | CAUAUUAA G CACUUUAA | 8025 | UUAAAGUG GCCGAAAGGCGAGUGAGGUCU UUAAUAUG | 18697 |
| 5515 | CACUUUAA G CUCCUUGA | 8026 | UCAAGGAG GCCGAAAGGCGAGUGAGGUCU UUAAAGUG | 18698 |
| 5524 | CUCCUUGA G UAAAAAGG | 8027 | CCUUUUUA GCCGAAAGGCGAGUGAGGUCU UCAAGGAG | 18699 |
| 5532 | GUAAAAAG G UGGUAUGU | 8028 | ACAUACCA GCCGAAAGGCGAGUGAGGUCU CUUUUUAC | 18700 |
| 5535 | AAAAGGUG G UAUGUAAU | 8029 | AUUACAUA GCCGAAAGGCGAGUGAGGUCU CACCUUUU | 18701 |
| 5539 | GGUGGUAU G UAAUUUAU | 8030 | AUAAAUUA GCCGAAAGGCGAGUGAGGUCU AUACCACC | 18702 |
| 5548 | UAAUUUAU G CAAGGUAU | 8032 | AUACCUUG GCCGAAAGGCGAGUGAGGUCU AUAAAUUA | 18703 |
| 5553 | UAUGCAAG G UAUUUCUC | 8033 | GAGAAAUA GCCGAAAGGCGAGUGAGGUCU CUUGCAUA | 18704 |
| 5564 | UUUCUCCA G UUGGGACU | 8034 | AGUCCCAA GCCGAAAGGCGAGUGAGGUCU UGGAGAAA | 18705 |
| 5583 | GGAUAUUA G UUAAUGAG | 8037 | CUCAUUAA GCCGAAAGGCGAGUGAGGUCU UAAUAUCC | 18706 |
| 5591 | GUUAAUGA G CCAUCACU | 8039 | AGUGAUGG GCCGAAAGGCGAGUGAGGUCU UCAUUAAC | 18707 |
| 5609 | GAAGAAAA G CUUUGAAA | 8040 | AAAAUGGG GCCGAAAGGCGAGUGAGGUCU UUUUCUUC | 18708 |
| 5623 | UUUCAACU G CUUUGAAA | 8042 | UUUCAAAG GCCGAAAGGCGAGUGAGGUCU AGUUGAAA | 18709 |
| 5635 | UGAAACUU G CCUGGGGU | 8044 | ACCCCAGG GCCGAAAGGCGAGUGAGGUCU AAGUUUCA | 18710 |
| 5642 | UGCCUGGG G UCUGAGCA | 8045 | UGCUCAGA GCCGAAAGGCGAGUGAGGUCU CCCAGGCA | 18711 |
| 5648 | GGGUCUGA G CAUGAUGG | 8046 | CCAUCAUG GCCGAAAGGCGAGUGAGGUCU UCAGACCC | 18712 |
| 5672 | GAGACAGG G UAGGAAAG | 8050 | CUUUCCUA GCCGAAAGGCGAGUGAGGUCU CCUGUCUC | 18713 |
| 5682 | AGGAAAGG G CGCCUACU | 8051 | AGUAGGCG GCCGAAAGGCGAGUGAGGUCU CCUUUCCU | 18714 |
| 5684 | GAAAGGGC G CCUACUCU | 8052 | AGAGUAGG GCCGAAAGGCGAGUGAGGUCU GCCCUUUC | 18715 |
| 5698 | UCUUCAGG G UCUAAAGA | 8053 | UCUUUAGA GCCGAAAGGCGAGUGAGGUCU CCUGAAGA | 18716 |
| 5711 | AAGAUCAA G UGGGCCUU | 8055 | AAGGCCCA GCCGAAAGGCGAGUGAGGUCU UUGAUCUU | 18717 |
| 5715 | UCAAGUGG G CCUUGGAU | 8056 | AUCCAAGG GCCGAAAGGCGAGUGAGGUCU CCACUUGA | 18718 |
| 5725 | CUUGGAUC G CUAAGCUG | 8058 | CAGCUUAG GCCGAAAGGCGAGUGAGGUCU GAUCCAAG | 18719 |
| 5730 | AUCGCUAA G CUGGCUCU | 8059 | AGAGCCAG GCCGAAAGGCGAGUGAGGUCU UUAGCGAU | 18720 |
| 5734 | CUAAGCUG G CUCUGUUU | 8060 | AAACAGAG GCCGAAAGGCGAGUGAGGUCU CAGCUUAG | 18721 |
| 5739 | CUGGCUCU G UUUGAUGC | 8061 | GCAUCAAA GCCGAAAGGCGAGUGAGGUCU AGAGCCAG | 18722 |
| 5746 | UGUUUGAU G CUAUUUAU | 8063 | AUAAAUAG GCCGAAAGGCGAGUGAGGUCU AUCAAACA | 18723 |
| 5755 | CUAUUUAU G CAAGUUAG | 8064 | CUAACUUG GCCGAAAGGCGAGUGAGGUCU AUAAAUAG | 18724 |
| 5759 | UUAUGCAA G UUAGGGUC | 8065 | GACCCUAA GCCGAAAGGCGAGUGAGGUCU UUGCAUAA | 18725 |
| 5765 | AAGUUAGG G UCUAUGUA | 8066 | UACAUAGA GCCGAAAGGCGAGUGAGGUCU CCUAACUU | 18726 |
| 5771 | GGGUCUAU G UAUUUAGG | 8067 | CCUAAAUA GCCGAAAGGCGAGUGAGGUCU AUAGACCC | 18727 |
| 5782 | UUUAGGAU G CGCCUACU | 8069 | AGUAGGCG GCCGAAAGGCGAGUGAGGUCU AUCCUAAA | 18728 |
| 5784 | UAGGAUGC G CCUACUCU | 8070 | AGAGUAGG GCCGAAAGGCGAGUGAGGUCU GCAUCCUA | 18729 |
| 5798 | UCUUCAGG G UCUAAAGA | 8053 | UCUUUAGA GCCGAAAGGCGAGUGAGGUCU CCUGAAGA | 18716 |
| 5811 | AAGAUCAA G UGGGCCUU | 8055 | AAGGCCCA GCCGAAAGGCGAGUGAGGUCU UUGAUCUU | 18717 |
| 5815 | UCAAGUGG G CCUUGGAU | 8056 | AUCCAAGG GCCGAAAGGCGAGUGAGGUCU CCACUUGA | 18718 |
| 5825 | CUUGGAUC G CUAAGCUG | 8058 | CAGCUUAG GCCGAAAGGCGAGUGAGGUCU GAUCCAAG | 18719 |
| 5830 | AUCGCUAA G CUGGCUCU | 8059 | AGAGCCAG GCCGAAAGGCGAGUGAGGUCU UUAGCGAU | 18720 |
| 5834 | CUAAGCUG G CUCUGUUU | 8060 | AAACAGAG GCCGAAAGGCGAGUGAGGUCU CAGCUUAG | 18721 |
| 5839 | CUGGCUCU G UUUGAUGC | 8061 | GCAUCAAA GCCGAAAGGCGAGUGAGGUCU AGAGCCAG | 18722 |
| 5846 | UGUUUGAU G CUAUUUAU | 8063 | AUAAAUAG GCCGAAAGGCGAGUGAGGUCU AUCAAACA | 18723 |
| 5855 | CUAUUUAU G CAAGUUAG | 8064 | CUAACUUG GCCGAAAGGCGAGUGAGGUCU AUAAAUAG | 18724 |
| 5859 | UUAUGCAA G UUAGGGUC | 8065 | GACCCUAA GCCGAAAGGCGAGUGAGGUCU UUGCAUAA | 18725 |
| 5865 | AAGUUAGG G UCUAUGUA | 8066 | UACAUAGA GCCGAAAGGCGAGUGAGGUCU CCUAACUU | 18726 |
| 5871 | GGGUCUAU G UAUUUAGG | 8067 | CCUAAAUA GCCGAAAGGCGAGUGAGGUCU AUAGACCC | 18727 |
| 5882 | UUUAGGAU G UCUGCACC | 8072 | GGUGCAGA GCCGAAAGGCGAGUGAGGUCU AUCCUAAA | 18730 |
| 5886 | GGAUGUCU G CACCUUCU | 8073 | UACAUAGA GCCGAAAGGCGAGUGAGGUCU AGAAGGUG | 18731 |
| 5895 | CACCUUCU G CAGCCAGU | 8074 | ACUGGCUG GCCGAAAGGCGAGUGAGGUCU AGAAGGUG | 18732 |
| 5898 | CUUCUGCA G CCAGUCAG | 8075 | CUGACUGG GCCGAAAGGCGAGUGAGGUCU UGCAGAAG | 18733 |
| 5902 | UGCAGCCA G UCAGAAGC | 8076 | GCUUCUGA GCCGAAAGGCGAGUGAGGUCU UGGCUGCA | 18734 |
| 5909 | AGUCAGAA G CUGGAGAG | 8077 | CUCUCCAG GCCGAAAGGCGAGUGAGGUCU UUCUGACU | 18735 |
| 5918 | CUGGAGAG G CAACAGUG | 8078 | CACUGUUG GCCGAAAGGCGAGUGAGGUCU CUCUCCAG | 18736 |
| 5924 | AGGCAACA G UGGAUUGC | 8080 | GCAAUCCA GCCGAAAGGCGAGUGAGGUCU UGUUGCCU | 18737 |
| 5931 | AGUGGAUU G CUGCUUCU | 8082 | AGAAGCAG GCCGAAAGGCGAGUGAGGUCU AAUCCACU | 18738 |
| 5934 | GGAUUGCU G CUUCUUGG | 8083 | CCAAGAAG GCCGAAAGGCGAGUGAGGUCU AGCAAUCC | 18739 |
| 5951 | GGAGAAGA G UAUGCUUC | 8084 | GAAGCAUA GCCGAAAGGCGAGUGAGGUCU UCUUCUCC | 18740 |
| 5955 | AAGAGUAU G CUUCCUUU | 8085 | AAAGGAAG GCCGAAAGGCGAGUGAGGUCU AUACUCUU | 18741 |
| 5971 | UUAUCCAU G UAAUUUAA | 8086 | UUAAAUUA GCCGAAAGGCGAGUGAGGUCU AUGGAUAA | 18742 |
| 5982 | AUUUAACU G UAGAACCU | 8089 | AGGUUCUA GCCGAAAGGCGAGUGAGGUCU AGUUAAAU | 18743 |
| 5993 | GAACCUGA G CUCUAAGU | 8091 | ACUUAGAG GCCGAAAGGCGAGUGAGGUCU UCAGGUUC | 18744 |
| 6000 | AGCUCUAA G UAACCGAA | 8092 | UUCGGUUA GCCGAAAGGCGAGUGAGGUCU UUAGAGCU | 18745 |
| 6013 | CGAAGAAU G UAUGCCUC | 8095 | GAGGCAUA GCCGAAAGGCGAGUGAGGUCU AUUCUUCG | 18746 |
| 6017 | GAAUGUAU G CCUCUGUU | 8096 | AACAGAGG GCCGAAAGGCGAGUGAGGUCU AUACAUUC | 18747 |
| 6023 | AUGCCUCU G UUCUUAUG | 8097 | CAUAAGAA GCCGAAAGGCGAGUGAGGUCU AGAGGCAU | 18748 |
| 6031 | GUUCUUAU G UGCCACAU | 8098 | AUGUGGCA GCCGAAAGGCGAGUGAGGUCU AUAAGAAC | 18749 |
| 6033 | UCUUAUGU G CCACAUCC | 8099 | GGAUGUGG GCCGAAAGGCGAGUGAGGUCU AGAGGCAU | 18750 |
| 6044 | ACAUCCUU G UUUAAAGG | 8100 | CCUUUAAA GCCGAAAGGCGAGUGAGGUCU AAGGAUGU | 18751 |
| 6052 | GUUUAAAG G CUCUCUGU | 8101 | ACAGAGAG GCCGAAAGGCGAGUGAGGUCU CUUUAAAC | 18752 |
| 6059 | GGCUCUCU G UAUGAAGA | 8102 | UCUUCAUA GCCGAAAGGCGAGUGAGGUCU AAGAGAGC | 18753 |
| 6077 | AUGGGACC G UCAUCAGC | 8105 | GCUGAUGA GCCGAAAGGCGAGUGAGGUCU GGUCCCAU | 18754 |
| 6084 | CGUCAUCA G CACAUUCC | 8106 | GGAAUGUG GCCGAAAGGCGAGUGAGGUCU UGAUGACG | 18755 |
| 6096 | AUUCCCUA G UGAGCCUA | 8107 | UAGGCUCA GCCGAAAGGCGAGUGAGGUCU UAGGGAAU | 18756 |
| 6100 | CCUAGUGA G CCUACUGG | 8108 | CCAGUAGG GCCGAAAGGCGAGUGAGGUCU UCACUAGG | 18757 |
| 6108 | GCCUACUG G CUCCUGGC | 8109 | GCCAGGAG GCCGAAAGGCGAGUGAGGUCU CAGUAGGC | 18758 |
| 6115 | GGCUCCUG G CAGCGGCU | 8110 | AGCCGCUG GCCGAAAGGCGAGUGAGGUCU CAGGAGCC | 18759 |
| 6118 | UCCUGGCA G CGGCUUUU | 8111 | AAAAGCCG GCCGAAAGGCGAGUGAGGUCU UGCCAGGA | 18760 |
| 6121 | UGGCAGCG G CUUUUGUG | 8112 | CACAAAAG GCCGAAAGGCGAGUGAGGUCU CGCUGCCA | 18761 |
| 6127 | CGGCUUUU G UGGAAGAC | 8113 | GUCUUCCA GCCGAAAGGCGAGUGAGGUCU AAAAGCCG | 18762 |
| 6142 | ACUCACUA G CCAGAAGA | 8115 | UCUUCUGG GCCGAAAGGCGAGUGAGGUCU UAGUGAGU | 18763 |
| 6156 | AGAGAGGA G UGGGACAG | 8116 | CUGUCCCA GCCGAAAGGCGAGUGAGGUCU UCCUCUCU | 18764 |
| 6164 | GUGGGACA G UCCUCUCC | 8118 | GGAGAGGA GCCGAAAGGCGAGUGAGGUCU UGUCCCAC | 18765 |
| 6197 | AAACAAAA G CAGGCUAG | 8122 | CUAGCCUG GCCGAAAGGCGAGUGAGGUCU UUUUGUUU | 18766 |
| 6201 | AAAAGCAG G CUAGAGCC | 8123 | GGCUCUAG GCCGAAAGGCGAGUGAGGUCU CUGCUUUU | 18767 |
| 6207 | AGGCUAGA G CCAGAAGA | 8124 | UCUUCUGG GCCGAAAGGCGAGUGAGGUCU UCUAGCCU | 18768 |
| 6230 | AAAUCUUU G UUGUUCCU | 8127 | AGGAACAA GCCGAAAGGCGAGUGAGGUCU AAAGAUUU | 18769 |
| 6233 | UCUUUGUU G UUCCUCUU | 8128 | AAGAGGAA GCCGAAAGGCGAGUGAGGUCU AACAAAGA | 18770 |
| 6254 | ACACAUAC G CAAACCAC | 8129 | GUGGUUUG GCCGAAAGGCGAGUGAGGUCU GUAUGUGU | 18771 |
| 6265 | AACCACCU G UGACAGCU | 8131 | AGCUGUCA GCCGAAAGGCGAGUGAGGUCU AGGUGGUU | 18772 |
| 6271 | CUGUGACA G CUGGCAAU | 8133 | AUUGCCAG GCCGAAAGGCGAGUGAGGUCU UGUCACAG | 18773 |
| 6275 | GACAGCUG G CAAUUUUA | 8134 | UAAAAUUG GCCGAAAGGCGAGUGAGGUCU CAGCUGUC | 18774 |
| 6292 | UAAAUCAG G UAACUGGA | 8137 | UCCAGUUA GCCGAAAGGCGAGUGAGGUCU CUGAUUUA | 18775 |
| 6306 | GGAAGGAG G UUAAACUC | 8139 | GAGUUUAA GCCGAAAGGCGAGUGAGGUCU CUCCUUCC | 18776 |
| 6333 | AGACCUCA G UCAAUUCU | 8142 | AGAAUUGA GCCGAAAGGCGAGUGAGGUCU UGAGGUCU | 18777 |
| 6377 | AGAUAAUA G CCCAGCAA | 8147 | UUGCUGGG GCCGAAAGGCGAGUGAGGUCU UAUUAUCU | 18778 |
| 6382 | AUAGCCCA G CAAAUAGU | 8148 | ACUAUUUG GCCGAAAGGCGAGUGAGGUCU UGGGCUAU | 18779 |
| 6389 | AGCAAAUA G UGAUAACA | 8150 | UGUUAUCA GCCGAAAGGCGAGUGAGGUCU UAUUUGCU | 18780 |
| 6410 | AAACCUUA G CUGUUCAU | 8155 | AUGAACAG GCCGAAAGGCGAGUGAGGUCU UAAGGUUU | 18781 |
| 6413 | CCUUAGCU G UUCAUGUC | 8156 | GACAUGAA GCCGAAAGGCGAGUGAGGUCU AGCUAAGG | 18782 |
| 6419 | CUGUUCAU G UCUUGAUU | 8157 | AAUCAAGA GCCGAAAGGCGAGUGAGGUCU AUGAACAG | 18783 |
| 6487 | AGAGAAAA G CAAAACCA | 8166 | UGGUUUUG GCCGAAAGGCGAGUGAGGUCU UUUUCUCU | 18784 |
| 6504 | UUAGAAUU G UUACUCAG | 8169 | CUGAGUAA GCCGAAAGGCGAGUGAGGUCU AAUUCUAA | 18785 |
| 6512 | GUUACUCA G CUCCUUCA | 8170 | UGAAGGAG GCCGAAAGGCGAGUGAGGUCU UGAGUAAC | 18786 |
| 6528 | AAACUCAG G UUUGUAGC | 8172 | GCUACAAA GCCGAAAGGCGAGUGAGGUCU CUGAGUUU | 18787 |
| 6532 | UCAGGUUU G UAGCAUAC | 8173 | GUAUGCUA GCCGAAAGGCGAGUGAGGUCU AAACCUGA | 18788 |
| 6535 | GGUUUGUA G CAUACAUG | 8174 | CAUGUAUG GCCGAAAGGCGAGUGAGGUCU UACAAACC | 18789 |
| 6545 | AUACAUGA G UCCAUCCA | 8175 | UGGAUGGA GCCGAAAGGCGAGUGAGGUCU UCAUGUAU | 18790 |
| 6557 | AUCCAUCA G UCAAAGAA | 8176 | UUCUUUGA GCCGAAAGGCGAGUGAGGUCU UGAUGGAU | 18791 |
| 6568 | AAAGAAUG G UUCCAUCU | 8178 | AGAUGGAA GCCGAAAGGCGAGUGAGGUCU CAUUCUUU | 18792 |
| 6580 | CAUCUGGA G UCUUAAUG | 8179 | CAUUAAGA GCCGAAAGGCGAGUGAGGUCU UCCAGAUG | 18793 |
| 6588 | GUCUUAAU G UAGAAAGA | 8181 | UCUUUCUA GCCGAAAGGCGAGUGAGGUCU AUUAAGAC | 18794 |
| 6610 | GGAGACUU G UAAUAAUG | 8184 | CAUUAUUA GCCGAAAGGCGAGUGAGGUCU AAGUCUCC | 18795 |
| 6620 | AAUAAUGA G CUAGUUAC | 8187 | GUAACUAG GCCGAAAGGCGAGUGAGGUCU UCAUUAUU | 18796 |
| 6624 | AUGAGCUA G UUACAAAG | 8188 | CUUUGUAA GCCGAAAGGCGAGUGAGGUCU UAGCUCAU | 18797 |
| 6632 | GUUACAAA G UGCUUGUU | 8189 | AACAAGCA GCCGAAAGGCGAGUGAGGUCU UUUGUAAC | 18798 |
| 6634 | UACAAAGU G CUUGUUCA | 8190 | UGAACAAG GCCGAAAGGCGAGUGAGGUCU ACUUUGUA | 18799 |
| 6638 | AAGUGCUU G UUCAUUAA | 8191 | UUAAUGAA GCCGAAAGGCGAGUGAGGUCU AAGCACUU | 18800 |
| 6651 | UUAAAAUA G CACUGAAA | 8193 | UUUCAGUG GCCGAAAGGCGAGUGAGGUCU UAUUUUAA | 18801 |
| 6698 | AAUCAUUU G CCAUUUAU | 8201 | AUAAAUGG GCCGAAAGGCGAGUGAGGUCU AAAUGAUU | 18802 |
| 6717 | CAAAAAUG G UUGGCACU | 8204 | AGUGCCAA GCCGAAAGGCGAGUGAGGUCU CAUUUUUG | 18803 |
| 6721 | AAUGGUUG G CACUAACA | 8205 | UGUUAGUG GCCGAAAGGCGAGUGAGGUCU CAACCAUU | 18804 |
| 6738 | AAGAACGA G CACUUCCU | 8208 | AGGAAGUG GCCGAAAGGCGAGUGAGGUCU UCGUUCUU | 18805 |
| 6753 | CUUUCAGA G UUUCUGAG | 8209 | CUCAGAAA GCCGAAAGGCGAGUGAGGUCU UCUGAAAG | 18806 |
| 6767 | GAGAUAAU G UACGUGGA | 8212 | UCCACGUA GCCGAAAGGCGAGUGAGGUCU AUUAUCUC | 18807 |
| 6771 | UAAUGUAC G UGGAACAG | 8213 | CUGUUCCA GCCGAAAGGCGAGUGAGGUCU GUACAUUA | 18808 |
| 6779 | GUGGAACA G UCUGGGUG | 8215 | CACCCAGA GCCGAAAGGCGAGUGAGGUCU UGUUCCAC | 18809 |
| 6785 | CAGUCUGG G UGGAAUGG | 8216 | CCAUUCCA GCCGAAAGGCGAGUGAGGUCU CCAGACUG | 18810 |
| 6795 | GGAAUGGG G CUGAAACC | 8218 | GGUUUCAG GCCGAAAGGCGAGUGAGGUCU CCCAUUCC | 18811 |
| 6806 | GAAACCAU G UGCAAGUC | 8220 | GACUUGCA GCCGAAAGGCGAGUGAGGUCU AUGGUUUC | 18812 |
| 6808 | AACCAUGU G CAAGUCUG | 8221 | CAGACUUG GCCGAAAGGCGAGUGAGGUCU ACAUGGUU | 18813 |
| 6812 | AUGUGCAA G UCUGUGUC | 8222 | GACACAGA GCCGAAAGGCGAGUGAGGUCU UUGCACAU | 18814 |
| 6816 | GCAAGUCU G UGUCUUGU | 8223 | ACAAGACA GCCGAAAGGCGAGUGAGGUCU AGACUUGC | 18815 |
| 6818 | AAGUCUGU G UCUUGUCA | 8224 | UGACAAGA GCCGAAAGGCGAGUGAGGUCU ACAGACUU | 18816 |
| 6823 | UGUUGUCA G UCCAAGAA | 8225 | UUCUUGGA GCCGAAAGGCGAGUGAGGUCU AAGACACA | 18817 |
| 6827 | UCUUGUCA G UCCAAGAA | 8226 | UUCUUGGA GCCGAAAGGCGAGUGAGGUCU UGACAAGA | 18818 |
| 6836 | UCCAAGAA G UGACACCG | 8227 | CGGUGUCA GCCGAAAGGCGAGUGAGGUCU UUCUUGGA | 18819 |
| 6849 | ACCGAGAU G UUAAUUUU | 8230 | AAAAUUAA GCCGAAAGGCGAGUGAGGUCU AUCUCGGU | 18820 |
| 6866 | AGGGACCC G UGCCUUGU | 8233 | ACAAGGCA GCCGAAAGGCGAGUGAGGUCU GGGUCCCU | 18821 |
| 6868 | GGACCCGU G CCUUGUUU | 8234 | AAACAAGG GCCGAAAGGCGAGUGAGGUCU ACGGGUCC | 18822 |
| 6873 | CGUGCCUU G UUUCCUAG | 8235 | CUAGGAAA GCCGAAAGGCGAGUGAGGUCU AAGGCACG | 18823 |
| 6881 | GUUUCCUA G CCCACAAG | 8236 | CUUGUGGG GCCGAAAGGCGAGUGAGGUCU UAGGAAAC | 18824 |
| 6893 | ACAAGAAU G CAAACAUC | 8238 | GAUGUUUG GCCGAAAGGCGAGUGAGGUCU AUUCUUGU | 18825 |
| 6914 | AGAUACUC G CUAGCCUC | 8242 | GAGGCUAG GCCGAAAGGCGAGUGAGGUCU GAGUAUCU | 18826 |
| 6918 | ACUCGCUA G CCUCAUUU | 8243 | AAAUGAGG GCCGAAAGGCGAGUGAGGUCU UAGCGAGU | 18827 |
| 6945 | AAGGAGGA G UGCAUCUU | 8246 | AAGAUGCA GCCGAAAGGCGAGUGAGGUCU UCCUCCUU | 18828 |
| 6947 | GGAGGAGU G CAUCUUUG | 8247 | CAAAGAUG GCCGAAAGGCGAGUGAGGUCU ACUCCUCC | 18829 |
| 6956 | CAUCUUUG G CCGACAGU | 8248 | ACUGUCGG GCCGAAAGGCGAGUGAGGUCU CAAAGAUG | 18830 |
| 6963 | GGCCGACA G UGGUGUAA | 8250 | UUACACCA GCCGAAAGGCGAGUGAGGUCU UGUCGGCC | 18831 |
| 6966 | CGACAGUG G UGUAACUG | 8251 | CAGUUACA GCCGAAAGGCGAGUGAGGUCU CACUGUCG | 18832 |
| 6968 | ACAGUGGU G UAACUGUG | 8252 | CACUUACA GCCGAAAGGCGAGUGAGGUCU CACUGUCG | 18833 |
| 6974 | GUGUAACU G UGUGUGUG | 8254 | CACACACA GCCGAAAGGCGAGUGAGGUCU AGUUACAC | 18834 |
| 6976 | GUAACUGU G UGUGUGUG | 8255 | CACACACA GCCGAAAGGCGAGUGAGGUCU ACAGUUAC | 18835 |
| 6978 | AACUGUGU G UGUGUGUG | 8256 | CACCCACA GCCGAAAGGCGAGUGAGGUCU ACACAGUU | 18836 |
| 6980 | CUGUGUGU G UGUGUGUG | 8257 | CACCCACA GCCGAAAGGCGAGUGAGGUCU ACACACAG | 18837 |
| 6982 | GUGUGUGU G UGUGUGUG | 8258 | CACACACA GCCGAAAGGCGAGUGAGGUCU ACACACAC | 18837 |
| 6984 | GUGUGUGU G UGUGUGUG | 8258 | CACACACA GCCGAAAGGCGAGUGAGGUCU ACACACAC | 18837 |
| 6986 | GUGUGUGU G UGUGUGUG | 8258 | CACACACA GCCGAAAGGCGAGUGAGGUCU ACACACAC | 18837 |
| 6988 | GUGUGUGU G UGUGUGUG | 8258 | CACACACA GCCGAAAGGCGAGUGAGGUCU ACACACAC | 18837 |
| 6990 | GUGUGUGU G UGUGUGUG | 8258 | CACACACA GCCGAAAGGCGAGUGAGGUCU ACACACAC | 18837 |
| 6992 | GUGUGUGU G UGUGUGUG | 8258 | CACACACA GCCGAAAGGCGAGUGAGGUCU ACACACAC | 18837 |
| 6994 | GUGUGUGU G UGUGUGUG | 8258 | CACACACA GCCGAAAGGCGAGUGAGGUCU ACACACAC | 18837 |
| 6996 | GUGUGUGU G UGUGUGUG | 8258 | CACACACA GCCGAAAGGCGAGUGAGGUCU ACACACAC | 18837 |
| 6998 | GUGUGUGU G UGUGUGUG | 8258 | CACACACA GCCGAAAGGCGAGUGAGGUCU ACACACAC | 18837 |
| 7000 | GUGUGUGU G UGUGUGUG | 8258 | CACACACA GCCGAAAGGCGAGUGAGGUCU ACACACAC | 18837 |
| 7002 | GUGUGUGU G UGUGUGUG | 8258 | CACACACA GCCGAAAGGCGAGUGAGGUCU ACACACAC | 18837 |
| 7004 | GUGUGUGU G UGUGUGUG | 8258 | CACACACA GCCGAAAGGCGAGUGAGGUCU ACACACAC | 18837 |
| 7006 | GUGUGUGU G UGUGUGUG | 8258 | CACACACA GCCGAAAGGCGAGUGAGGUCU ACACACAC | 18837 |
| 7008 | GUGUGUGU G UGUGUGGG | 8259 | CACACACA GCCGAAAGGCGAGUGAGGUCU ACACACAC | 18839 |
| 7010 | GUGUGUGU G UGUGGGUG | 8260 | CACACACA GCCGAAAGGCGAGUGAGGUCU ACACACAC | 18840 |
| 7012 | GUGUGUGU G UGGGUGUG | 8261 | CACACCCA GCCGAAAGGCGAGUGAGGUCU ACACACAC | 18841 |
| 7016 | GUGUGUGG G UGUGGGUG | 8262 | CACCCACA GCCGAAAGGCGAGUGAGGUCU CCACACAC | 18842 |
| 7018 | GUGUGGGU G UGGGUGUA | 8263 | UACACCCA GCCGAAAGGCGAGUGAGGUCU ACCCACAC | 18843 |
| 7022 | GGGUGUGG G UGUAUGUG | 8264 | CACAUACA GCCGAAAGGCGAGUGAGGUCU CCACACCC | 18844 |
| 7024 | GUGUGGGU G UAUGUGUG | 8265 | CACACAUA GCCGAAAGGCGAGUGAGGUCU ACCCACAC | 18845 |
| 7028 | GGGUGUAU G UGUGUUUU | 8266 | AAAACACA GCCGAAAGGCGAGUGAGGUCU AUACACCC | 18846 |
| 7030 | GUGUAUGG G UGUUUUGU | 8267 | ACAAAACA GCCGAAAGGCGAGUGAGGUCU ACAUACAC | 18847 |
| 7032 | GUAUGUGU G UUUUGUGC | 8268 | GCACAAAA GCCGAAAGGCGAGUGAGGUCU ACACAUAC | 18848 |
| 7037 | UGUGUUUU G UGCAUAAC | 8269 | GUUAUGCA GCCGAAAGGCGAGUGAGGUCU AAAACACA | 18849 |
| 7039 | UGUUUUGU G CAUAACUA | 8270 | UAGUUAUG GCCGAAAGGCGAGUGAGGUCU ACAAAACA | 18850 |
| 7071 | AUUUUAAA G UUACUUUU | 8274 | AAAAGUAA GCCGAAAGGCGAGUGAGGUCU UUUAAAAU | 18851 |
| 7099 | GAAUAUAU G CUACAGAU | 8277 | AUCUGUAG GCCGAAAGGCGAGUGAGGUCU AUAUAUUC | 18852 |
| 7122 | CAGACAUG G UUUGGUCC | 8281 | GGACCAAA GCCGAAAGGCGAGUGAGGUCU CAUGUCUG | 18853 |
| 7127 | AUGGUUUG G UCCUAUAU | 8282 | AUAUAGGA GCCGAAAGGCGAGUGAGGUCU CAAACCAU | 18854 |
| 7141 | UAUUUCUA G UCAUGAUG | 8283 | CAUCAUGA GCCGAAAGGCGAGUGAGGUCU UAGAAAUA | 18855 |
| 7153 | UGAUGAAU G UAUUUUGU | 8286 | ACAAAAUA GCCGAAAGGCGAGUGAGGUCU AUUCAUCA | 18856 |
| 7160 | UGUAUUUU G UAUACCAU | 8287 | AUGGUAUA GCCGAAAGGCGAGUGAGGUCU AAAAUACA | 18857 |
| 7210 | UGGGAUUU G UAAUCGUA | 8292 | UACGAUUA GCCGAAAGGCGAGUGAGGUCU AAAUCCCA | 18858 |
| 7216 | UUGUAAUC G UACCAACU | 8294 | AGUUGGUA GCCGAAAGGCGAGUGAGGUCU GAUUACAA | 18859 |
| 7240 | UAAACUUG G CAACUGCU | 8299 | AGCAGUUG GCCGAAAGGCGAGUGAGGUCU CAAGUUUA | 18860 |
| 7246 | UGGCAACU G CUUUUAUG | 8301 | CAUAAAAG GCCGAAAGGCGAGUGAGGUCU AGUUGCCA | 18861 |
| 7254 | GCUUUUAU G UUCUGUCU | 8302 | AGACAGAA GCCGAAAGGCGAGUGAGGUCU AUAAAAGC | 18862 |
| 7259 | UAUGUUCU G UCUCCUUC | 8303 | GAAGGAGA GCCGAAAGGCGAGUGAGGUCU AGAACAUA | 18863 |
| 7305 | AAGAAAAA G CUCUUUUU | 8308 | AAAAAGAG GCCGAAAGGCGAGUGAGGUCU UUUUUCUU | 18864 |
| 7343 | UUAUCCUU G UUUAGAGC | 8312 | GCUCUAAA GCCGAAAGGCGAGUGAGGUCU AAGGAUAA | 18865 |
| 7350 | UGUUUAGA G CAGAGAAA | 8313 | UUUCUCUG GCCGAAAGGCGAGUGAGGUCU UCUAAACA | 18866 |
| 7381 | UUGAAAUG G UCUCAAAA | 8317 | UUUUGAGA GCCGAAAGGCGAGUGAGGUCU CAUUUCAA | 18867 |
| 7394 | AAAAAAUU G CUAAAUAU | 8319 | AUAUUUAG GCCGAAAGGCGAGUGAGGUCU AAUUUUUU | 18868 |
| 7422 | AACUAAAU G UUAGUUUA | 8324 | UAAACUAA GCCGAAAGGCGAGUGAGGUCU AUUUAGUU | 18869 |
| 7426 | AAAUGUUA G UUUAGCUG | 8325 | CAGCUAAA GCCGAAAGGCGAGUGAGGUCU UAACAUUU | 18870 |
| 7431 | UUAGUUUA G CUGAUUGU | 8326 | ACAAUCAG GCCGAAAGGCGAGUGAGGUCU UAAACUAA | 18871 |
| 7438 | AGCUGAUU G UAUGGGGU | 8328 | ACCCCAUA GCCGAAAGGCGAGUGAGGUCU AAUCAGCU | 18872 |
| 7445 | UGUAUGGG G UUUUCGAA | 8329 | UUCGAAAA GCCGAAAGGCGAGUGAGGUCU CCCAUACA | 18873 |
| 7467 | CACUUUUU G UUUGUUUU | 8331 | AAAACAAA GCCGAAAGGCGAGUGAGGUCU AAAAAGUG | 18874 |
| 7471 | UUUUGUUU G UUUUACCU | 8332 | AGGUAAAA GCCGAAAGGCGAGUGAGGUCU AAACAAAA | 18875 |
| 7491 | UCACAACU G UGUAAAUU | 8334 | AAUUUACA GCCGAAAGGCGAGUGAGGUCU AGUUGUGA | 18876 |
| 7493 | ACAACUGU G UAAAUUGC | 8335 | GCAAUUUA GCCGAAAGGCGAGUGAGGUCU ACAGUUGU | 18877 |
| 7500 | UGUAAAUU G CCAAUAAU | 8337 | AUUAUUGG GCCGAAAGGCGAGUGAGGUCU AAUUUACA | 18878 |
| 7513 | UAAUUCCU G UCCAUGAA | 8340 | UUCAUGGA GCCGAAAGGCGAGUGAGGUCU AGGAAUUA | 18879 |
| 7525 | AUGAAAAU G CAAAUUAU | 8342 | AUAAUUUG GCCGAAAGGCGAGUGAGGUCU AUUUUCAU | 18880 |
| 7537 | AUUAUCCA G UGUAGAUA | 8344 | UAUCUACA GCCGAAAGGCGAGUGAGGUCU UGGAUAAU | 18881 |
| 7539 | UAUCCAGU G UAGAUAUA | 8345 | UAUAUCUA GCCGAAAGGCGAGUGAGGUCU ACUGGAUA | 18882 |
| 7573 | GGAUAUUG G CUAGUUUU | 8349 | AAAACUAG GCCGAAAGGCGAGUGAGGUCU CAAUAUCC | 18883 |
| 7577 | AUUGGCUA G UUUUGCCU | 8350 | AGGCAAAA GCCGAAAGGCGAGUGAGGUCU UAGCCAAU | 18884 |
| 7582 | CUAGUUUU G CCUUUAUU | 8351 | AAUAAAGG GCCGAAAGGCGAGUGAGGUCU AAAACUAG | 18885 |
| 7593 | UUUAUUAA G CAAAUUCA | 8352 | UGAAUUUG GCCGAAAGGCGAGUGAGGUCU UUAAUAAA | 18886 |
| 7607 | UCAUUUCA G CCUGAAUG | 8354 | CAUUCAGG GCCGAAAGGCGAGUGAGGUCU UGAAAUGA | 18887 |
| 7615 | GCCUGAAU G UCUGCCUA | 8356 | UAGGCAGA GCCGAAAGGCGAGUGAGGUCU AUUCAGGC | 18888 |
| 7619 | GAAUGUCU G CCUAUAUA | 8357 | UAUAUAGG GCCGAAAGGCGAGUGAGGUCU AGACAUUC | 18889 |
| 7634 | UAUUCUCU G CUCUUUGU | 8358 | ACAAAGAG GCCGAAAGGCGAGUGAGGUCU AGAGAAUA | 18890 |
| 7641 | UGCUCUUU G UAUUCUCC | 8359 | GGAGAAUA GCCGAAAGGCGAGUGAGGUCU AAAGAGCA | 18891 |
| 7659 | UUGAACCC G UUAAAACA | 8361 | UGUUUUAA GCCGAAAGGCGAGUGAGGUCU GGGUUCAA | 18892 |
| 7672 | AACAUCCU G UGGCACUC | 8363 | GAGUGCCA GCCGAAAGGCGAGUGAGGUCU AGGAUGUU | 18893 |

Input Sequence = HSFLT.
Cut Site = G/Y
Arm Length = 8.
Core Sequence = GCcgaaagGCGaGuCaaGGuCu
HSFLT (Human flt mRNA for receptor-related tyrosine kinase.; Acc# X51602; 7680 bp)

TABLE XX
Patient Demographics

| Dose cohort | | | | | | |
|---|---|---|---|---|---|---|
| (mg/m²) | Pt # | Age | Sex | Diagnosis | Status | Doses |
| 10 | 1001 | 49 | F | NSC Lung | PD | 29 |
| 10 | 1002 | 65 | F | liposarcoma | PD | 120 |
| 10 | 1003 | 49 | M | nasopharyngeal CA | WD | 109 |
| 30 | 1004* | | | | | |
| 30 | 1005 | 45 | F | melanoma (ocular) | PD | 113 |
| 30 | 1006 | 57 | M | colon | PD | 199 |
| 30 | 1007 | 39 | F | epitheliod hemangioendothelioma | PD | 198 |
| 100 | 1008 | 52 | M | adrenal ca | PD | 57 |
| 100 | 1009 | 44 | F | breast | PD | 35 |
| 100 | 1010 | 62 | F | renal | PD | 134 |
| 300 | 1011 | 24 | F | melanoma | PD | 31 |
| 300 | 1012 | 57 | M | renal cell | PD | 178 |
| 300 | 1013 | 53 | M | nasopharyngeal SCCA | PD | 29 |
| 300 | 1014 | 64 | F | peritoneal mesothelioma | stable | 236** |
| 100 | 1015 | 65 | M | melanoma | PD | 140 |
| 100 | 1016 | 77 | F | breast | stable | 231** |
| 100 | 1017 | | F | melanoma | PD | 35 |
| 100 | 1018* | | | | | |
| 100 | 1019 | 69 | F | endometrial sarcoma | stable | 212** |
| 100 | 1020 | 65 | M | carcinoid | PD | 124 |
| 100 | 1021 | 59 | M | gallbladder adeno carcinoma | PD | 34 |
| 100 | 1022* | | | | | |
| 100 | 1023 | 78 | F | breast | PD | 50 |
| 100 | 1024 | 40 | F | parotid adenocarcinoma | stable | 161** |
| 100 | 1025 | 52 | F | breast | WD | 71 |
| 100 | 1026 | 39 | F | breast | PD | 34 |
| 100 | 1027 | 55 | F | breast | PD | 36 |
| 100 | 1028 | 52 | M | melanoma | PD | 29 |
| 100 | 1029 | 38 | M | pancreatic | PD | 36 |
| 100 | 1030 | 83 | M | melanoma | PD | 41 |
| 100 | 1031 | 50 | M | medullary thyroid | WD | 108 |

PD = progressive disease;
WD = withdrawal.
4 patients remain on study.
*Inappropriate enrollment, patient withdrawn
**As of Jan. 11, 2000

TABLE XXI
Pharmacokinetic parameters of ANGIOZYME after bolus subcutaneous
administration.

| | 10 mg/m² | | 30 mg/m² | | 100 mg/m² | | 300 mg/m² | |
|---|---|---|---|---|---|---|---|---|
| | Mean | SD | Mean | SD | Mean | SD | Mean | SD |
| Day 1 | | | | | | | | |
| Cmax | 0.43 | 0.07 | 0.62 | 0.28 | 3.17 | 0.69 | 8.91 | 2.93 |
| (ug/mL) | | | | | | | | |
| AUCt (ug * | 2.60 | 1.43 | 6.04 | 2.70 | 34.14 | 2.28 | 89.87 | 21.68 |
| hr/mL) | | | | | | | | |
| AUCinf | 4.40 | 0.06 | 7.99 | 1.66 | 37.51 | 1.91 | 101.57 | 13.47 |
| (ug * hr/mL) | | | | | | | | |
| t (½) (hr) | 3.62 | 0.79 | 7.32 | 6.94 | 4.58 | 0.02 | 9.26 | 6.20 |
| CL/F | 2.24 | 0.08 | 3.73 | 0.92 | 2.96 | 0.61 | 2.99 | 0.43 |
| (L/hr/m²) | | | | | | | | |
| Day 29 | | | | | | | | |
| Cmax | 0.35 | 0.19 | 1.17 | 0.53 | 3.23 | 0.35 | 8.93 | 6.71 |
| (ug/mL) | | | | | | | | |
| AUCt (ug * | 2.11 | 1.31 | 7.29 | 1.16 | 31.87 | 1.91 | 119.42 | 65.84 |
| hr/mL) | | | | | | | | |
| AUCinf | 3.38 | 1.31 | 8.54 | 2.46 | 33.61 | 2.16 | 132.73 | 67.82 |
| (ug * hr/mL) | | | | | | | | |
| t (½) (hr) | 4.49 | 1.60 | 3.26 | 1.01 | 4.66 | 0.35 | 7.24 | 0.70 |
| CL/F | 2.49 | 1.48 | 3.69 | 0.94 | 3.21 | 0.56 | 2.72 | 1.40 |
| (L/hr/m²) | | | | | | | | |

TABLE XXII
Human KDR DNAzyme and Substrate sequence

| | | Seq ID | | Seq ID |
|---|---|---|---|---|
| Pos | Substrate | No | DNAzyme | No |
| 14 | GUCCCGGG A CCCCGGGA | 8364 | TCCCGGGG GGCTAGCTACAACGA CCCGGGAC | 18894 |
| 25 | CCGGGAGA G CGGUCAGU | 8365 | ACTGACCG GGCTAGCTACAACGA TCTCCCGG | 18895 |
| 28 | GGAGAGCG G UCAGUGUG | 8366 | CACACTGA GGCTAGCTACAACGA CGCTCTCC | 18896 |
| 32 | AGCGGUCA G UGUGUGGU | 8367 | ACCACACA GGCTAGCTACAACGA TGACCGCT | 18897 |
| 34 | CGGUCAGU G UGUGGUCG | 8368 | CGACCACA GGCTAGCTACAACGA ACTGACCG | 18898 |
| 36 | GUCAGUGU G UGGUCGCU | 8369 | AGCGACCA GGCTAGCTACAACGA ACACTGAC | 18899 |
| 39 | AGUGUGUG G UCGCUGCG | 8370 | CGCAGCGA GGCTAGCTACAACGA CACACACT | 18900 |
| 42 | GUGUGGUC G CUGCGUUU | 8371 | AAACGCAG GGCTAGCTACAACGA GACCACAC | 18901 |
| 45 | UGGUCGCU G CGUUUCCU | 8372 | AGGAAACG GGCTAGCTACAACGA AGCGACCA | 18902 |
| 47 | GUCGCUGC G UUUCCUCU | 8373 | AGAGGAAA GGCTAGCTACAACGA GCAGCGAC | 18903 |
| 56 | UUUCCUCU G CCUGCGCC | 8374 | GGCGCAGG GGCTAGCTACAACGA AGAGGAAA | 18904 |
| 60 | CUCUGCCU G CGCCGGGC | 8375 | GCCCGGCG GGCTAGCTACAACGA AGGCAGAG | 18905 |
| 62 | CUGCCUGC G CCGGGCAU | 8376 | ATGCCCGG GGCTAGCTACAACGA GCAGGCAG | 18906 |
| 67 | UGCGCCGG G CAUCACUU | 8377 | AAGTGATG GGCTAGCTACAACGA CCGGCGCA | 18907 |
| 69 | CGCCGGGC A UCACUUGC | 8378 | GCAAGTGA GGCTAGCTACAACGA GCCCGGCG | 18908 |
| 72 | CGGGCAUC A CUUGCGCG | 8379 | CGCGCAAG GGCTAGCTACAACGA GATGCCCG | 18909 |
| 76 | CAUCACUU G CGCGCCGC | 8380 | GCGGCGCG GGCTAGCTACAACGA AAGTGATG | 18910 |
| 78 | UCACUUGC G CGCCGCAG | 8381 | CTGCGGCG GGCTAGCTACAACGA GCAAGTGA | 18911 |
| 80 | ACUUGCGC G CCGCAGAA | 8382 | TTCTGCGG GGCTAGCTACAACGA GCGCAAGT | 18912 |
| 83 | UGCGCGCC G CAGAAAGU | 8383 | ACTTTCTG GGCTAGCTACAACGA GGCGCGCA | 18913 |
| 90 | CGCAGAAA G UCCGUCUG | 8384 | CAGACGGA GGCTAGCTACAACGA TTTCTGCG | 18914 |
| 94 | GAAAGUCC G UCUGGCAG | 8385 | CTGCCAGA GGCTAGCTACAACGA GGACTTTC | 18915 |
| 99 | UCCGUCUG G CAGCCUGG | 8386 | CCAGGCTG GGCTAGCTACAACGA CAGACGGA | 18916 |
| 102 | GUCUGGCA G CCUGGAUA | 8387 | TATCCAGG GGCTAGCTACAACGA TGCCAGAC | 18917 |
| 108 | CAGCCUGG A UAUCCUCU | 8388 | AGAGGATA GGCTAGCTACAACGA CCAGGCTG | 18918 |
| 110 | GCCUGGAU A UCCUCUCC | 8389 | GGAGAGGA GGCTAGCTACAACGA ATCCAGGC | 18919 |
| 120 | CCUCUCCU A CCGGCACC | 8390 | GGTGCCGG GGCTAGCTACAACGA AGGAGAGG | 18920 |
| 124 | UCCUACCG G CACCCGCA | 8391 | TGCGGGTG GGCTAGCTACAACGA CGGTAGGA | 18921 |
| 126 | CUACCGGC A CCCGCAGA | 8392 | TCTGCGGG GGCTAGCTACAACGA GCCGGTAG | 18922 |
| 130 | CGGCACCC G CAGACGCC | 8393 | GGCGTCTG GGCTAGCTACAACGA GGGTGCCG | 18923 |
| 134 | ACCCGCAG A CGCCCCUG | 8394 | CAGGGGCG GGCTAGCTACAACGA CTGCGGGT | 18924 |
| 136 | CCGCAGAC G CCCCUGCA | 8395 | TGCAGGGG GGCTAGCTACAACGA GTCTGCGG | 18925 |
| 142 | ACGCCCCU G CAGCCGCC | 8396 | GGCGGCTG GGCTAGCTACAACGA AGGGGCGT | 18926 |
| 145 | CCCCUGCA G CCGCCGGU | 8397 | CCCGGGCG GGCTAGCTACAACGA CGACCGGC | 18927 |
| 148 | CUGCAGCC G CCGGUCGG | 8398 | CCGACCGG GGCTAGCTACAACGA GGCTGCAG | 18928 |
| 152 | AGCCGCCG G UCGGCGCC | 8399 | GGCGCCGA GGCTAGCTACAACGA CGGCGGCT | 18929 |
| 156 | GCCGGUCG G CGCCCGGG | 8400 | CCCGGGCG GGCTAGCTACAACGA CGACCGGC | 18930 |
| 158 | CGGUCGGC G CCCGGGCU | 8401 | AGCCCGGG GGCTAGCTACAACGA GCCGACCG | 18931 |
| 164 | GCGCCCGG G CUCCCUAG | 8402 | CTAGGGAG GGCTAGCTACAACGA CCGGGCGC | 18932 |
| 172 | GCUCCCUA G CCCUGUGC | 8403 | GCACAGGG GGCTAGCTACAACGA TAGGGAGC | 18933 |
| 177 | CUAGCCCU G UGCGCUCA | 8404 | TGAGCGCA GGCTAGCTACAACGA AGGGCTAG | 18934 |
| 179 | AGCCCUGU G CGCUCAAC | 8405 | GTTGAGCG GGCTAGCTACAACGA ACAGGGCT | 18935 |
| 181 | CCCUGUGC G CUCAACUG | 8406 | CAGTTGAG GGCTAGCTACAACGA GCACAGGG | 18936 |
| 186 | UGCGCUCA A CUGUCCUG | 8407 | CAGGACAG GGCTAGCTACAACGA TGACCGCA | 18937 |
| 189 | GCUCAACU G UCCUGCGC | 8408 | GCGCAGGA GGCTAGCTACAACGA AGTTGAGC | 18938 |
| 194 | ACUGUCCU G CGCUGCGG | 8409 | CCGCAGCG GGCTAGCTACAACGA AGGACAGT | 18939 |
| 196 | UGUCCUGC G CUGCGGGG | 8410 | CCCCGCAG GGCTAGCTACAACGA GCAGGACA | 18940 |
| 199 | CCUGCGCU G CGGGGUGC | 8411 | GCACCCCG GGCTAGCTACAACGA AGCGCAGG | 18941 |
| 204 | GCUGCGGG G UGCCGCGA | 8412 | TCGCGGCA GGCTAGCTACAACGA CCCGCAGC | 18942 |
| 206 | UGCGGGGU G CCGCGAGU | 8413 | ACTCGCGG GGCTAGCTACAACGA ACCCCGCA | 18943 |
| 209 | GGGGUGCC G CGAGUUCC | 8414 | GGAACTCG GGCTAGCTACAACGA GGCACCCC | 18944 |
| 213 | UGCCGCGA G UUCCACCU | 8415 | AGGTGGAA GGCTAGCTACAACGA TCGCGGCA | 18945 |
| 218 | CGAGUUCC A CCUCCGCG | 8416 | CGCGGAGG GGCTAGCTACAACGA GGAACTCG | 18946 |
| 224 | CCACCUCC G CGCCUCCU | 8417 | AGGAGGCG GGCTAGCTACAACGA GGAGGTGG | 18947 |
| 226 | ACCUCCGC G CCUCCUUC | 8418 | GAAGGAGG GGCTAGCTACAACGA GCGGAGGT | 18948 |
| 240 | UUCUCUAG A CAGGCGCU | 8419 | AGCGCCTG GGCTAGCTACAACGA CTAGAGAA | 18949 |
| 244 | CUAGACAG G CGCUGGGA | 8420 | TCCCAGCG GGCTAGCTACAACGA CTGTCTAG | 18950 |
| 246 | AGACAGGC G CUGGGAGA | 8421 | TCTCCCAG GGCTAGCTACAACGA GCCTGTCT | 18951 |
| 259 | GAGAAAGA A CCGGCUCC | 8422 | GGAGCCGG GGCTAGCTACAACGA TCTTTCTC | 18952 |
| 263 | AAGAACCG G CUCCCGAG | 8423 | CTCGGGAG GGCTAGCTACAACGA CGGTTCTT | 18953 |
| 271 | GCUCCCGA G UUCUGGGC | 8424 | GCCCAGAA GGCTAGCTACAACGA TCGGGAGC | 18954 |
| 278 | AGUUCUGG G CAUUUCGC | 8425 | GCGAAATG GGCTAGCTACAACGA CCAGAACT | 18955 |
| 280 | UUCUGGGC A UUUCGCCC | 8426 | GGGCGAAA GGCTAGCTACAACGA GCCCAGAA | 18956 |
| 285 | GGCUCGAG G CCCGGCUC | 8427 | GAGCCGGG GGCTAGCTACAACGA GAAATGCC | 18957 |
| 290 | UUCGCCCG G CUCGAGGU | 8428 | ACCTCGAG GGCTAGCTACAACGA CGGGCGAA | 18958 |
| 297 | GGCUCGAG G UGCAGGAU | 8429 | ATCCTGCA GGCTAGCTACAACGA CTCGAGCC | 18959 |
| 299 | CUCGAGGU G CAGGAUGC | 8430 | GCATCCTG GGCTAGCTACAACGA ACCTCGAG | 18960 |
| 304 | GGUGCAGG A UGCAGAGC | 8431 | GCTCTGCA GGCTAGCTACAACGA CCTGCACC | 18961 |
| 306 | UGCAGGAU G CAGAGCAA | 8432 | TTGCTCTG GGCTAGCTACAACGA ATCCTGCA | 18962 |
| 311 | GAUGCAGA G CAAGGUGC | 8433 | GCACCTTG GGCTAGCTACAACGA TCTGCATC | 18963 |
| 316 | AGAGCAAG G UGCUGCUG | 8434 | CAGCAGCA GGCTAGCTACAACGA CTTGCTCT | 18964 |
| 318 | AGCAAGGU G CUGCUGGC | 8435 | GCCAGCAG GGCTAGCTACAACGA ACCTTGCT | 18965 |
| 321 | AAGGUGCU G CUGGCCGU | 8436 | ACGGCCAG GGCTAGCTACAACGA AGCACCTT | 18966 |
| 325 | UGCUGCUG G CCGUCGCC | 8437 | GGCGACGG GGCTAGCTACAACGA CAGCAGCA | 18967 |
| 328 | UGCUGGCC G UCGCCCUG | 8438 | CAGGGCGA GGCTAGCTACAACGA GGCCAGCA | 18968 |
| 331 | UGGCCGUC G CCCUGUGG | 8439 | CCACAGGG GGCTAGCTACAACGA GACGGCCA | 18969 |
| 336 | GUCGCCCU G UGGCUCUG | 8440 | CAGAGCCA GGCTAGCTACAACGA AGGGCGAC | 18970 |
| 339 | GCCCUGUG G CUCUGCGU | 8441 | ACGCAGAG GGCTAGCTACAACGA CACAGGGC | 18971 |
| 344 | GUGGCUCU G CGUGGAGA | 8442 | TCTCCACG GGCTAGCTACAACGA AGAGCCAC | 18972 |
| 346 | GGCUCUGC G UGGAGACC | 8443 | GGTCTCCA GGCTAGCTACAACGA GCAGAGCC | 18973 |
| 352 | GCGUGGAG A CCCGGGCC | 8444 | GGCCCGGG GGCTAGCTACAACGA CTCCACGC | 18974 |
| 358 | AGACCCGG G CCGCCUCU | 8445 | AGAGGCGG GGCTAGCTACAACGA CCGGGTCT | 18975 |
| 361 | CCCGGGCC G CCUCUGUG | 8446 | CACAGAGG GGCTAGCTACAACGA GGCCCGGG | 18976 |
| 367 | CCGCCUCU G UGGGUUUG | 8447 | CAAACCCA GGCTAGCTACAACGA AGAGGCGG | 18977 |
| 371 | CUCUGUGG G UUUGCCUA | 8448 | TAGGCAAA GGCTAGCTACAACGA CCACAGAG | 18978 |
| 375 | GUGGGUUU G CCUAGUGU | 8449 | ACACTAGG GGCTAGCTACAACGA AAACCCAC | 18979 |
| 380 | UUUGCCUA G UGUUUCUC | 8450 | GAGAAACA GGCTAGCTACAACGA TAGGCAAA | 18980 |
| 382 | UGCCUAGU G UUUCUCUU | 8451 | AAGAGAAA GGCTAGCTACAACGA ACTAGGCA | 18981 |
| 392 | UUCUCUUG A UCUGCCCA | 8452 | TGGGCAGA GGCTAGCTACAACGA CAAGAGAA | 18982 |
| 396 | CUUGAUCU G CCCAGGCU | 8453 | AGCCTGGG GGCTAGCTACAACGA AGATCAAG | 18983 |
| 402 | CUGCCCAG G CUCAGCAU | 8454 | ATGCTGAG GGCTAGCTACAACGA CTGGGCAG | 18984 |
| 407 | CAGGCUCA G CAUACAAA | 8455 | TTTGTATG GGCTAGCTACAACGA TGAGCCTG | 18985 |
| 409 | GGCUCAGC A UACAAAAA | 6138 | TTTTTGTA GGCTAGCTACAACGA GCTGAGCC | 18986 |
| 411 | CUCAGCAU A CAAAAAGA | 1519 | TCTTTTTG GGCTAGCTACAACGA ATGCTGAG | 18987 |
| 419 | ACAAAAAG A CAUACUUA | 8456 | TAAGTATG GGCTAGCTACAACGA CTTTTTGT | 18988 |
| 421 | AAAAAGAC A UACUUACA | 6140 | TGTAAGTA GGCTAGCTACAACGA GTCTTTTT | 18989 |
| 423 | AAAGACAU A CUUACAAU | 1520 | ATTGTAAG GGCTAGCTACAACGA ATGTCTTT | 18990 |
| 427 | ACAUACUU A CAAUUAAG | 1522 | CTTAATTG GGCTAGCTACAACGA AAGTATGT | 18991 |
| 430 | UACUUACA A UUAAGGCU | 8457 | AGCCTTAA GGCTAGCTACAACGA TGTAAGTA | 18992 |
| 436 | CAAUUAAG G CUAAUACA | 8458 | TGTATTAG GGCTAGCTACAACGA CTTAATTG | 18993 |
| 440 | UAAGGCUA A UACAACUC | 8459 | GAGTTGTA GGCTAGCTACAACGA TAGCCTTA | 18994 |
| 442 | AGGCUAAU A CAACUCUU | 1526 | AAGAGTTG GGCTAGCTACAACGA ATTAGCCT | 18995 |
| 445 | CUAAUACA A CUCUUCAA | 8460 | TTGAAGAG GGCTAGCTACAACGA TGTATTAG | 18996 |
| 454 | CUCUUCAA A UUACUUGC | 8461 | GCAAGTAA GGCTAGCTACAACGA TTGAAGAG | 18997 |
| 457 | UUCAAAUU A CUUGCAGG | 1531 | CCTGCAAG GGCTAGCTACAACGA AATTTGAA | 18998 |
| 461 | AAUUACUU G CUUGCAGG | 8462 | CCTGCAAG GGCTAGCTACAACGA AATTTGAA | 18999 |
| 468 | UGCAGGGG A CAGAGGGA | 8463 | GTCCCCTG GGCTAGCTACAACGA AAGTAATT | 19000 |
| 476 | ACAGAGGG A CUUGGACU | 8464 | TCCCTCTG GGCTAGCTACAACGA CCCCTGCA | 19001 |
| 482 | GGACUUGG A CUGGCUUU | 8465 | AGTCCAAG GGCTAGCTACAACGA CCCTCTGT | 19002 |
| 486 | UUGGACUG G CUUUGGCC | 8466 | AAAGCCAG GGCTAGCTACAACGA CCAAGTCC | 19003 |
| 492 | UGGCUUUG G CCCAAUAA | 8467 | TTATTGGG GGCTAGCTACAACGA CAGTCCAA | 19004 |
| 497 | UUGGCCCA A UAAUCAGA | 8468 | TCTGATTA GGCTAGCTACAACGA TGGGCCAA | 19005 |
| 500 | GCCCAAUA A UCAGAGUG | 8469 | CACTCTGA GGCTAGCTACAACGA TATTGGGC | 19006 |
| 506 | UAAUCAGA C UGGCAGUG | 8470 | GCTCACTG GGCTAGCTACAACGA TCTGATTA | 19007 |
| 509 | UCAGAGUC G CAGUGAGC | 8471 | GCTCACTG GGCTAGCTACAACGA CACTCTGA | 19008 |
| 512 | GAGUGGCA G UGAGCAAA | 8472 | TTTGCTCA GGCTAGCTACAACGA TGCCACTC | 19009 |
| 516 | GGCAGUGA C CAAAGGGU | 8473 | ACCCTTTG GGCTAGCTACAACGA TCACTGCC | 19010 |
| 523 | AGCAAAGG C UGGAGGUG | 8474 | CACCTCCA GGCTAGCTACAACGA CCTTTGCT | 19011 |
| 529 | GGGUGGAG C UGACUGAG | 8475 | CTCAGTCA GGCTAGCTACAACGA CTCCACCC | 19012 |
| 532 | UGGAGGUG A CUGACUGC | 8476 | GCACTCAG GGCTAGCTACAACGA CACCTCCA | 19013 |
| 537 | GUGACUGA C UGCAGCGA | 8477 | TCGCTGCA GGCTAGCTACAACGA TCAGTCAC | 19014 |
| 539 | GACUGAGU C CAGCGAUG | 8478 | CATCGCTG GGCTAGCTACAACGA ACTCAGTC | 19015 |
| 542 | UGAGUGCA C CGAUGGCC | 8479 | GGCCATCG GGCTAGCTACAACGA TGCACTCA | 19016 |
| 545 | GUGCAGCG A UGGCCUCU | 8480 | AGAGGCCA GGCTAGCTACAACGA CGCTGCAC | 19017 |
| 548 | CAGCGAUG C CCUCUUCU | 8481 | AGAAGAGG GGCTAGCTACAACGA CATCGCTG | 19018 |
| 557 | CCUCUUCU C UAAGACAC | 8482 | GTGTCTTA GGCTAGCTACAACGA AGAAGAGG | 19019 |
| 562 | UCUGUAAC A CACUCACA | 8483 | TGTGAGTG GGCTAGCTACAACGA CTTACAGA | 19020 |
| 564 | UGUAAGAC A CUCACAAU | 6166 | ATTGTGAG GGCTAGCTACAACGA GTCTTACA | 19021 |
| 568 | AGACACUC A CAAUUCCA | 6168 | TGGAATTG GGCTAGCTACAACGA GAGTGTCT | 19022 |
| 571 | CACUCACA A UUCCAAAA | 8484 | TTTTGGAA GGCTAGCTACAACGA TGTGAGTG | 19023 |
| 580 | UUCCAAAA C UGAUCGCA | 8485 | TCCGATCA GGCTAGCTACAACGA TTTTGGAA | 19024 |
| 583 | CAAAAGUG A UCGGAAAU | 8486 | ATTTCCGA GGCTAGCTACAACGA CACTTTTG | 19025 |
| 590 | GAUCGGAA A UGACACUG | 8487 | CAGTGTCA GGCTAGCTACAACGA TTCCGATC | 19026 |
| 593 | CGGAAAUG A CACUGGAG | 8488 | CTCCAGTG GGCTAGCTACAACGA CATTTCCG | 19027 |
| 595 | GAAAUGAC A CUGGAGCC | 6172 | GGCTCCAG GGCTAGCTACAACGA GTCATTTC | 19028 |
| 601 | ACACUGGA C CCUACAAG | 8489 | CTTGTAGG GGCTAGCTACAACGA TCCAGTGT | 19029 |
| 605 | UGGAGCCU A CAAGUGCU | 1546 | AGCACTTG GGCTAGCTACAACGA AGGCTCCA | 19030 |
| 609 | GCCUACAA C UGCUUCUA | 8490 | TAGAAGCA GGCTAGCTACAACGA TTGTAGGC | 19031 |
| 611 | CUACAAGU C CUUCUACC | 8491 | GGTAGAAG GGCTAGCTACAACGA ACTTGTAG | 19032 |
| 617 | GUGCUUCU A CCGGGAAA | 1549 | TTTCCCGG GGCTAGCTACAACGA AGAAGCAC | 19033 |
| 625 | ACCGGGAA A CUGACUUG | 8492 | CAAGTCAG GGCTAGCTACAACGA TTCCCGGT | 19034 |
| 629 | GGAAACUG A CUUGGCCU | 8493 | AGGCCAAG GGCTAGCTACAACGA CAGTTTCC | 19035 |
| 634 | CUGACUUG C CCUCGGUC | 8494 | GACCGAGG GGCTAGCTACAACGA CAAGTCAG | 19036 |
| 640 | UGGCCUCG G UCAUUUAU | 8495 | ATAAATGA GGCTAGCTACAACGA CGAGGCCA | 19037 |
| 643 | CCUCGGUC A UUUAUGUC | 6184 | GACATAAA GGCTAGCTACAACGA GACCGAGG | 19038 |
| 647 | GGUCAUUU A UGUCUAUG | 1555 | CATAGACA GGCTAGCTACAACGA AAATGACC | 19039 |
| 649 | UCAUUUAU G UCUAUGUU | 8496 | AACATAGA GGCTAGCTACAACGA ATAAATGA | 19040 |
| 653 | UUAUGUCU A UGUUCAAG | 1557 | CTTGAACA GGCTAGCTACAACGA AGACATAA | 19041 |
| 655 | AUGUCUAU G UUCAAGAU | 8497 | ATCTTGAA GGCTAGCTACAACGA ATAGACAT | 19042 |
| 662 | UGUUCAAG A UUACAGAU | 8498 | ATCTGTAA GGCTAGCTACAACGA CTTGAACA | 19043 |
| 665 | UCAAGAUU A CAGAUCUC | 1561 | GAGATCTG GGCTAGCTACAACGA AATCTTGA | 19044 |
| 669 | GAUUACAG A UCUCCAUU | 8499 | AATGGAGA GGCTAGCTACAACGA CTGTAATC | 19045 |
| 675 | AGAUCUCC A UUUAUUGC | 6190 | GCAATAAA GGCTAGCTACAACGA GGAGATCT | 19046 |
| 679 | CUCCAUUU A UUGCUUCU | 1566 | AGAAGCAA GGCTAGCTACAACGA AAATGGAG | 19047 |
| 682 | CAUUUAUU G CUUCUGUU | 8500 | AACAGAAG GGCTAGCTACAACGA AATAAATG | 19048 |
| 688 | UUGCUUCU G UUAGUGAC | 8501 | GTCACTAA GGCTAGCTACAACGA AGAAGCAA | 19049 |
| 692 | UUCUGUUA G UGACCAAC | 8502 | GTTGGTCA GGCTAGCTACAACGA TAACAGAA | 19050 |
| 695 | UGUUAGUG A CCAACAUG | 8503 | CATGTTGG GGCTAGCTACAACGA CACTAACA | 19051 |
| 699 | AGUGACCA A CAUGGAGU | 8504 | ACTCCATG GGCTAGCTACAACGA TGGTCACT | 19052 |
| 701 | UGACCAAC A UGGAGUCG | 6195 | CGACTCCA GGCTAGCTACAACGA GTTGGTCA | 19053 |
| 706 | AACAUGGA G UCGUGUAC | 8505 | GTACACGA GGCTAGCTACAACGA TCCATGTT | 19054 |
| 709 | AUGGAGUC G UGUACAUU | 8506 | AATGTACA GGCTAGCTACAACGA GACTCCAT | 19055 |
| 711 | GGAGUCGU G UACAUUAC | 8507 | GTAATGTA GGCTAGCTACAACGA ACGACTCC | 19056 |
| 713 | AGUCGUGU A CAUUACUG | 1573 | CAGTAATG GGCTAGCTACAACGA ACACGACT | 19057 |
| 715 | UCGUGUAC A UUACUGAG | 6196 | CTCAGTAA GGCTAGCTACAACGA GTACACGA | 19058 |
| 718 | UGUACAUU A CUGAGAAC | 1575 | GTTCTCAG GGCTAGCTACAACGA AATGTACA | 19059 |
| 725 | UACUGAGA A CAAAAACA | 8508 | TGTTTTTG GGCTAGCTACAACGA TCTCAGTA | 19060 |
| 731 | GAACAAAA A CAAAACUG | 8509 | CAGTTTTG GGCTAGCTACAACGA TTTTGTTC | 19061 |
| 736 | AAAACAAA A CUGUGGUG | 8510 | CACCACAG GGCTAGCTACAACGA TTTGTTTT | 19062 |
| 739 | ACAAAACU G UGGUGAUU | 8511 | AATCACCA GGCTAGCTACAACGA AGTTTTGT | 19063 |
| 742 | AAACUGUG G UGAUUCCA | 8512 | TGGAATCA GGCTAGCTACAACGA CACAGTTT | 19064 |
| 745 | CUGUGGUG A UUCCAUGU | 8513 | ACATGGAA GGCTAGCTACAACGA CACCACAG | 19065 |
| 750 | GUGAUUCC A UGUCUCGG | 6202 | CCGAGACA GGCTAGCTACAACGA GGAATCAC | 19066 |
| 752 | GAUUCCAU G UCUCGGGU | 8514 | ACCCGAGA GGCTAGCTACAACGA ATGGAATC | 19067 |
| 759 | UGUCUCGG G UCCAUUUC | 8515 | GAAATGGA GGCTAGCTACAACGA CCGAGACA | 19068 |
| 763 | UCGGGUCC A UUUCAAAU | 6205 | ATTTGAAA GGCTAGCTACAACGA GGACCCGA | 19069 |
| 770 | CAUUUCAA A UCUCAACG | 8516 | CGTTGAGA GGCTAGCTACAACGA TTGAAATG | 19070 |
| 776 | AAAUCUCA A CGUGUCAC | 8517 | GTGACACG GGCTAGCTACAACGA TGAGATTT | 19071 |
| 778 | AUCUCAAC G UGUCACUU | 8518 | AAGTGACA GGCTAGCTACAACGA GTTGAGAT | 19072 |
| 780 | CUCAACGU G UCACUUUG | 8519 | CAAAGTGA GGCTAGCTACAACGA ACGTTGAG | 19073 |
| 783 | AACGUGUC A CUUUGUGC | 6209 | GCACAAAG GGCTAGCTACAACGA GACACGTT | 19074 |
| 788 | GUCACUUU G UGCAAGAU | 8520 | ATCTTGCA GGCTAGCTACAACGA AAAGTGAC | 19075 |
| 790 | CACUUUGU G CAAGAUAC | 8521 | GTATCTTG GGCTAGCTACAACGA ACAAAGTG | 19076 |
| 795 | UGUGCAAG A UACCCAGA | 8522 | TCTGGGTA GGCTAGCTACAACGA CTTGCACA | 19077 |
| 797 | UGCAAGAU A CCCAGAAA | 1589 | TTTCTGGG GGCTAGCTACAACGA ATCTTGCA | 19078 |
| 810 | GAAAAGAG A UUUGUUCC | 8523 | GGAACAAA GGCTAGCTACAACGA CTCTTTTC | 19079 |
| 814 | AGAGAUUU G UUCCUGAU | 8524 | ATCAGGAA GGCTAGCTACAACGA AAATCTCT | 19080 |
| 821 | UGUUCCUG A UGGUAACA | 8525 | TGTTACCA GGCTAGCTACAACGA CAGGAACA | 19081 |
| 824 | UCCUGAUG G UAACAGAA | 8526 | TTCTGTTA GGCTAGCTACAACGA CATCAGGA | 19082 |
| 827 | UGAUGGUA A CAGAAUUU | 8527 | AAATTCTG GGCTAGCTACAACGA TACCATCA | 19083 |
| 832 | GUAACAGA A UUUCCUGG | 8528 | CCAGGAAA GGCTAGCTACAACGA TCTGTTAC | 19084 |
| 842 | UUCCUGGG A CAGCAAGA | 8529 | TCTTGCTG GGCTAGCTACAACGA CCCAGGAA | 19085 |
| 843 | CUGGGACA G CAAGAAGG | 8530 | CCTTCTTG GGCTAGCTACAACGA TGTCCCAG | 19086 |
| 854 | CAAGAAGG G CUUUACUA | 8531 | TAGTAAAG GGCTAGCTACAACGA CCTTCTTG | 19087 |
| 859 | AGGGCUUU A CUAUUCCC | 1600 | GGCAATAG GGCTAGCTACAACGA AAAGCCCT | 19088 |
| 862 | GCUUUACU A UUCCCAGC | 1601 | GCTGGGAA GGCTAGCTACAACGA AGTAAACC | 19089 |
| 869 | UAUUCCCA G CUACAUGA | 8532 | TCATGTAG GGCTAGCTACAACGA TGGGAATA | 19090 |
| 872 | UCCCAGCU A CAUGAUCA | 1604 | TGATCATG GGCTAGCTACAACGA AGCTGGGA | 19091 |
| 874 | CCAGCUAC A UGAUCAGC | 6228 | GCTGATCA GGCTAGCTACAACGA GTAGCTGG | 19092 |
| 877 | GCUACAUG A UCAGCUAU | 8533 | ATAGCTGA GGCTAGCTACAACGA CATGTAGC | 19093 |
| 881 | CAUGAUCA G CUAUGCUG | 8534 | CAGCATAG GGCTAGCTACAACGA TGATCATG | 19094 |
| 884 | GAUCAGCU A UGCUGGCA | 1606 | TGCCAGCA GGCTAGCTACAACGA AGCTGATC | 19095 |
| 886 | UCAGCUAU C CUGGCAUG | 8535 | CATOCCAG GGCTAGCTACAACGA ATAGCTGA | 19096 |
| 890 | CUAUGCUG G CAUGGUCU | 8536 | AGACCATG GGCTAGCTACAACGA CACCATAG | 19097 |
| 892 | AUGCUGGC A UGGUCUUC | 6232 | GAAGACCA GGCTAGCTACAACGA GCCAGCAT | 19098 |
| 895 | CUGGCAUG C UCUUCUGU | 8537 | ACAGAAGA GGCTAGCTACAACGA CATGCCAC | 19099 |
| 902 | GGUCUUCU G UGAAGCAA | 8538 | TTGCTTCA GGCTAGCTACAACGA AGAAGACC | 19100 |
| 907 | UCUGUGAA G CAAAAAUU | 8539 | AATTTTTG GGCTAGCTACAACGA TTCACAGA | 19101 |
| 913 | AAGCAAAA A UUAAUGAU | 8540 | ATCATTAA GGCTAGCTACAACGA TTTTGCTT | 19102 |
| 917 | AAAAAUUA A UCAUGAAA | 8541 | TTTCATCA GGCTAGCTACAACGA TAATTTTT | 19103 |
| 920 | AAUUAAUC A UGAAACUU | 8542 | AACTTTCA GGCTAGCTACAACGA CATTAATT | 19104 |
| 926 | UGAUCAAA G UUACCAGU | 8543 | ACTGGTAA GGCTAGCTACAACGA TTTCATCA | 19105 |
| 929 | UGAAAGUU A CCAGUCUA | 1613 | TACACTGC GGCTAGCTACAACGA AACTTTCA | 19106 |
| 933 | AGUUACCA G UCUAUUAU | 8544 | ATAATACA GGCTAGCTACAACGA TGGTAACT | 19107 |
| 937 | ACCACUCU A UUAUGUAC | 1615 | CTACATAA GGCTAGCTACAACGA AGACTGGT | 19108 |
| 940 | AGUCUAUU A UCUACAUA | 1617 | TATGTACA GGCTAGCTACAACGA AATACACT | 19109 |
| 942 | UCUAUUAU C UACAUACU | 8545 | ACTATGTA GGCTAGCTACAACGA ATAATAGA | 19110 |
| 944 | UAUUAUCU A CAUAGUUG | 1618 | CAACTATG GGCTAGCTACAACGA ACATAATA | 19111 |
| 946 | UUAUGUAC A UACUUGUC | 6239 | GACAACTA GGCTAGCTACAACGA CTACATAA | 19112 |
| 949 | UGUACAUA G UUGUCCUU | 8546 | AACGACAA GGCTAGCTACAACGA TATGTACA | 19113 |
| 952 | ACAUAGUU G UCGUUCUA | 8547 | TACAACCA GGCTAGCTACAACGA AACTATGT | 19114 |
| 955 | UAGUUGUC G UUGUACGG | 8548 | CCCTACAA GGCTAGCTACAACGA CACAACTA | 19115 |
| 958 | UUGUCGUU C UAGGGUAU | 8549 | ATACCCTA GGCTAGCTACAACGA AACGACAA | 19116 |
| 963 | GUUGUACG G UAUAGGAU | 8550 | ATCCTATA GGCTAGCTACAACGA CCTACAAC | 19117 |
| 965 | UGUAGGGU A UAGGAUUU | 1624 | AAATCCTA GGCTAGCTACAACGA ACCCTACA | 19118 |
| 970 | GCUAUAGC A UUUAUGAU | 8551 | ATCATAAA GGCTAGCTACAACGA CCTATACC | 19119 |
| 974 | UAGCAUUU A UGAUGUGC | 1628 | CCACATCA GGCTAGCTACAACGA AAATCCTA | 19120 |
| 977 | GAUUUAUG A UGUCGUUC | 8552 | CAACCACA GGCTAGCTACAACGA CATAAATC | 19121 |
| 979 | UUUAUCAU C UCGUUCUC | 8553 | CAGAACCA GGCTAGCTACAACGA ATCATAAA | 19122 |
| 982 | AUGAUCUG C UUCUCACU | 8554 | ACTCACAA GGCTAGCTACAACGA CACATCAT | 19123 |
| 989 | GGUUCUGA C UCCGUCUC | 8555 | CACACGGA GGCTAGCTACAACGA TCACAACC | 19124 |
| 993 | CUCACUCC C UCUCAUCC | 8556 | CCATCACA GGCTAGCTACAACGA CCACTCAC | 19125 |
| 998 | UCCCUCUC A UCCAAUUC | 6243 | CAATTCCA GGCTAGCTACAACGA CACACCCA | 19126 |
| 1003 | CUCAUCCA A UUCAACUA | 8557 | TACTTCAA GGCTAGCTACAACGA TCCATCAC | 19127 |
| 1008 | CCAAUUCA A CUAUCUCU | 8558 | ACACATAC GGCTAGCTACAACGA TCAATTCC | 19128 |
| 1011 | AUUCAACU A UCUCUUCC | 1635 | CCAACACA GGCTAGCTACAACGA ACTTCAAT | 19129 |
| 1015 | AACUAUCU C UUGCACAA | 8559 | TTCTCCAA GGCTAGCTACAACGA ACATACTT | 19130 |
| 1026 | CCACAAAA C CUUCUCUU | 8560 | AACACAAC GGCTAGCTACAACGA TTTTCTCC | 19131 |
| 1030 | AAAACCUU C UCUUAAAU | 8561 | ATTTAACA GGCTAGCTACAACGA AACCTTTT | 19132 |
| 1037 | UCUCUUAA A UUGUACAC | 8562 | CTCTACAA GGCTAGCTACAACGA TTAACACA | 19133 |
| 1040 | CUUAAAUu C UACACCAA | 8563 | TTCCTCTA GGCTAGCTACAACGA AATTTAAC | 19134 |
| 1042 | UAAAUUGU A CAGCAAGA | 1643 | TCTTGCTG GGCTAGCTACAUCGA ACAATTTA | 19135 |
| 1045 | AUUCUACA G CAAGAACU | 8564 | AGTTCTTC GGCTAGCTACAACGA TGTACAAT | 19136 |
| 1051 | CAGCAAGA A CUGAACUA | 8565 | TAGTTCAG GGCTAGCTACAACGA TCTTGCTG | 19137 |
| 1056 | AGAACUGA A CUAAAUGU | 8566 | ACATTTAG GGCTAGCTACAACGA TCAGTTCT | 19138 |
| 1061 | UCAACUAA A UGUCGGGA | 8567 | TCCCCACA GGCTAGCTACAACGA TTAGTTCA | 19139 |
| 1063 | AACUAAAU C UGGGCAUU | 8568 | AATCCCCA GGCTAGCTACAACGA ATTTAGTT | 19140 |
| 1069 | AUGUGUGC A UUCACUUC | 8569 | CAAGTCAA GGCTAGCTACAACGA CCCCACAT | 19141 |
| 1073 | GGCCAUUG A CUUCAACU | 8570 | AGTTGAAC GGCTAGCTACAACGA CAATCCCC | 19142 |
| 1079 | UGACUUCA A CUGCCAAU | 8571 | ATTCCCAG GGCTAGCTACAACGA TGAAGTCA | 19143 |
| 1086 | AACUCCCA A UACCCUUC | 8572 | CAAGCGTA GGCTAGCTACAACGA TCCCAGTT | 19144 |
| 1088 | CUCGGAAU A CCCUUCUU | 1648 | AAGAACGG GGCTAGCTACAACGA ATTCCCAG | 19145 |
| 1101 | UCUUCCAA G CAUCACCA | 8573 | TGCTCATG GGCTAGCTACAACGA TTCGAACA | 19146 |
| 1103 | UUCGAACC A UCAGCAUA | 6259 | TATCCTGA GGCTAGCTACAUCGA GCTTCGAA | 19147 |
| 1107 | AACCAUCA C CAUAACAA | 8574 | TTCTTATG GGCTAGCTACAACGA TGATCCTT | 19148 |
| 1109 | CCAUCACC A UAAGAAAC | 6261 | CTTTCTTA GGCTAGCTACAACGA GCTCATGC | 19149 |
| 1116 | CAUAAGAA A CUUCUAAA | 8575 | TTTACAAC GGCTAGCTACAACGA TTCTTATG | 19150 |
| 1120 | AGAAACUU C UAAACCCA | 8576 | TCGCTTTA GGCTAGCTACAACGA AACTTTCT | 19151 |
| 1124 | ACUUCUAA A CCGACACC | 8577 | GCTCTCGG GGCTAGCTACAACGA TTACAACT | 19152 |
| 1130 | AAACCCAC A CCUAAAAA | 8578 | TTTTTAGC GGCTAGCTACAACGA CTCGGTTT | 19153 |
| 1138 | ACCUAAAA A CCCAGUCU | 8579 | ACACTCGC GGCTAGCTACAACGA TTTTAGGT | 19154 |
| 1143 | AAAACCCA C UCUGGCAC | 8580 | CTCCCACA GGCTAGCTACAACGA TCCCTTTT | 19155 |
| 1151 | CUCUGGCA C UGAGAUCA | 8581 | TCATCTCA GGCTAGCTACAACGA TCCCACAC | 19156 |
| 1156 | GGACUGAC A UGAACAAA | 8582 | TTTCTTCA GGCTAGCTACAACGA CTCACTCC | 19157 |
| 1164 | AUGAAGAA A UUUUUCAC | 8583 | CTCAAAAA GGCTAGCTACAACGA TTCTTCAT | 19158 |
| 1172 | AUUUUUGA G CACCUUAA | 8584 | TTAACGTG GGCTAGCTACAACGA TCAAAAAT | 19159 |
| 1174 | UUUUGACC A CCUUAACU | 6270 | ACTTAAGC GGCTAGCTACAACGA CCTCAAAA | 19160 |
| 1180 | GCACCUUA A CUAUAGAU | 8585 | ATCTATAG GGCTAGCTACAACGA TAACGTCC | 19161 |
| 1183 | CCUUAACU A UACAUGCU | 1665 | ACCATCTA GGCTAGCTACAACGA AGTTAACC | 19162 |
| 1187 | AACUAUAC A UGGUGUAA | 8586 | TTACACCA GGCTAGCTACAACGA CTATAGTT | 19163 |
| 1190 | UAUAGAUC C UGUAACCC | 8587 | CGGTTACA GGCTAGCTACAACGA CATCTATA | 19164 |
| 1192 | UAGAUGGU C UAACCCGG | 8588 | CCGCCTTA GGCTAGCTACAACGA ACCATCTA | 19165 |
| 1195 | AUCGUCUA A CCCGGACU | 8589 | ACTCCGGC GGCTAGCTACAACGA TACACCAT | 19166 |
| 1202 | AACCCGGA C UCACCAAG | 8590 | CTTCGTCA GGCTAGCTACAACGA TCCGCGTT | 19167 |
| 1205 | CCGCAGUC A CCAAGCAU | 8591 | ATCCTTCC GGCTAGCTACAACGA CACTCCGC | 19168 |
| 1212 | CACCAAGG A UUGUACAC | 8592 | GTGTACAA GGCTAGCTACAACGA CCTTCGTC | 19169 |
| 1215 | CAAGGAUU C UACACCUG | 8593 | CAGGTGTA GCCTAGCTACAACGA AATCCTTG | 19170 |
| 1217 | ACGAUUCU A CACCUCUG | 1669 | CACACCTG CGCTAGCTACAACGA ACAATCCT | 19171 |
| 1219 | GAUUCUAC A CCUGUGCA | 6278 | TGCACAGC GGCTAGCTACAACGA CTACAATC | 19172 |
| 1223 | GUACACCU C UGCAGCAU | 8594 | ATGCTCCA GGCTAGCTACAACGA AGCTGTAC | 19173 |
| 1225 | ACACCUGU C CAGCAUCC | 8595 | GGATGCTG CCCTAGCTACAACGA ACAGCTCT | 19174 |
| 1228 | CCUGUGCA C CAUCCAGU | 8596 | ACTGGATG CCCTAGCTACAACGA TCCACAGC | 19175 |
| 1230 | UCUGCACC A UCCACUGG | 6282 | CCACTCGA CGCTAGCTACAACGA CCTCCACA | 19176 |
| 1235 | ACCAUCCA C UGGGCUCA | 8597 | TCAGCCCA GCCTAGCTACAACGA TCCATGCT | 19177 |
| 1239 | UCCAGUCC C CUCAUGAC | 8598 | GTCATCAC CGCTAGCTACAACGA CCACTCGA | 19178 |
| 1243 | CUCCGCUG A UGACCAAC | 8599 | CTTCCTCA GCCTAGCTACAACGA CAGCCCAC | 19179 |
| 1246 | GCCUGAUC A CCAACAAG | 8600 | CTTCTTGC CGCTAGCTACAACGA CATCAGCC | 19180 |
| 1256 | CAAGAAGA A CACCACAU | 8601 | ATGTCCTC GGCTAGCTACAACGA TCTTCTTG | 19181 |
| 1259 | CAACAACA C CACAUUUC | 8602 | CAAATGTC CCCTAGCTACAACGA TGTTCTTC | 19182 |
| 1261 | ACAACACC A CAUUUCUC | 6289 | CACAAATC GCCTAGCTACAACGA CCTCTTCT | 19183 |
| 1263 | AACAGCAC A UUUGUCAG | 6290 | CTGACAAA GGCTAGCTACAACGA GTGCTGTT | 19184 |
| 1267 | GCACAUUU G UCAGGGUC | 8603 | GACCCTGA GGCTAGCTACAACGA AAATGTGC | 19185 |
| 1273 | UUGUCAGG G UCCAUGAA | 8604 | TTCATGGA GGCTAGCTACAACGA CCTGACAA | 19186 |
| 1277 | CAGGGUCC A UGAAAAAC | 6293 | GTTTTTCA GGCTAGCTACAACGA GGACCCTG | 19187 |
| 1284 | CAUGAAAA A CCUUUUGU | 8605 | ACAAAAGG GGCTAGCTACAACGA TTTTCATG | 19188 |
| 1291 | AACCUUUU G UUGCUUUU | 8606 | AAAAGCAA GGCTAGCTACAACGA AAAAGGTT | 19189 |
| 1294 | CUUUUGUU C CUUUUGGA | 8607 | TCCAAAAG GGCTAGCTACAACGA AACAAAAG | 19190 |
| 1304 | UUUUGGAA C UGGCAUGG | 8608 | CCATGCCA GGCTAGCTACAACGA TTCCAAAA | 19191 |
| 1307 | UGGAAGUG G CAUGGAAU | 8609 | ATTCCATG GGCTAGCTACAACGA CACTTCCA | 19192 |
| 1309 | GAAGUGGC A UGGAAUCU | 6297 | AGATTCCA CGCTAGCTACAACGA CCCACTTC | 19193 |
| 1314 | GGCAUGCA A UCUCUGGU | 8610 | ACCAGAGA GGCTAGCTACAACGA TCCATGCC | 19194 |
| 1321 | AAUCUCUC C UGGAAGCC | 8611 | GCCTTCCA GGCTAGCTACAACGA CAGACATT | 19195 |
| 1327 | UGGUGGAA C CCACGGUG | 8612 | CACCGTGG GGCTAGCTACAACGA TTCCACCA | 19196 |
| 1330 | UCGAAGCC A CGCUGGCG | 6301 | CCCCACCC GGCTAGCTACAACGA CGCTTCCA | 19197 |
| 1333 | AAGCCACG C UGCGGCAG | 8613 | CTCCCCCA GGCTAGCTACAACGA CGTGCCTT | 19198 |
| 1341 | GUGGCGGA C CGUGUCAG | 8614 | CTCACACG GGCTAGCTACAACGA TCCCCCAC | 19199 |
| 1343 | CGGCCAGC C UCUCAGAA | 8615 | TTCTGACA GGCTAGCTACAACGA GCTCCCCC | 19200 |
| 1345 | GCGAGCGU C UCAGAAUC | 8616 | GATTCTGA GGCTAGCTACAACGA ACGCTCCC | 19201 |
| 1351 | GUGUCACA A UCCCUGCG | 8617 | CCCAGCGA GGCTAGCTACAACGA TCTGACAC | 19202 |
| 1357 | GAAUCCCU G CCAACUAC | 8618 | CTACTTCG GGCTAGCTACAACGA ACGGATTC | 19203 |
| 1362 | CCUGCGAA G UACCUUGG | 8619 | CCAAGGTA GGCTAGCTACAACGA TTCCCAGG | 19204 |
| 1364 | UGCGAAGU A CCUUGGUU | 1686 | AACCAACG GGCTAGCTACAACGA ACTTCCCA | 19205 |
| 1370 | GUACCUUG G UUACCCAC | 8620 | CTGCGTAA GGCTAGCTACAACGA CAAGGTAC | 19206 |
| 1373 | CCUUGGUU A CCCACCCC | 1689 | CGGGTGGG GGCTAGCTACAACGA AACCAAGC | 19207 |
| 1377 | GGUUACCC A CCCCCAGA | 6310 | TCTGGGCG GGCTAGCTACAACGA GGCTAACC | 19208 |
| 1387 | CCCCAGAA A UAAAAUCG | 8621 | CCATTTTA GGCTAGCTACAACGA TTCTGGGG | 19209 |
| 1392 | GAAAUAAA A UGGUAUAA | 8622 | TTATACCA GGCTAGCTACAACGA TTTATTTC | 19210 |
| 1395 | AUAAAAUG G UAUAAAAA | 8623 | TTTTTATA GGCTAGCTACAACGA CATTTTAT | 19211 |
| 1397 | AAAAUGCU A UAAAAAUG | 1691 | CATTTTTA GGCTAGCTACAACGA ACCATTTT | 19212 |
| 1403 | GUAUAAAA A UGGAAUAC | 8624 | GTATTCCA GGCTAGCTACAACGA TTTTATAC | 19213 |
| 1408 | AAAAUCGA A UACCCCUU | 8625 | AACGGGTA GGCTAGCTACAACGA TCCATTTT | 19214 |
| 1410 | AAUGGAAU A CCCCUUGA | 1693 | TCAACGGG GGCTAGCTACAACGA ATTCCATT | 19215 |
| 1419 | CCCCUUGA C UCCAAUCA | 8626 | TGATTGGA GGCTAGCTACAACGA TCAAGCCG | 19216 |
| 1424 | UGACUCCA A UCACACAA | 8627 | TTCTGTGA GGCTAGCTACAACGA TCGACTCA | 19217 |
| 1427 | CUCCAAUC A CACAAUUA | 6322 | TAATTCTC GGCTAGCTACAACGA GATTGGAC | 19218 |
| 1429 | CCAAUCAC A CAAUUAAA | 6323 | TTTAATTC GGCTAGCTACAACGA GTCATTCC | 19219 |
| 1432 | AUCACACA A UUAAACCC | 8628 | CCCTTTAA GGCTAGCTACAACGA TCTCTCAT | 19220 |
| 1438 | CAAUUAAA C CCCCCCAU | 8629 | ATCCCCCC GGCTAGCTACAACGA TTTAATTG | 19221 |
| 1443 | AAAGCGCG C CAUCUACU | 8630 | AGTACATC GGCTAGCTACAACGA CCCCCTTT | 19222 |
| 1445 | AGCGGGCC A UCUACUCA | 6325 | TCAGTACA GGCTAGCTACAACGA CCCCCCCT | 19223 |
| 1447 | CCCCCCAU C UACUCACC | 8631 | CGTCACTA GGCTAGCTACAACGA ATCCCCCC | 19224 |
| 1449 | CCCCAUGU A CUCACCAU | 1699 | ATCCTCAG GGCTAGCTACAACGA ACATCCCC | 19225 |
| 1453 | AUCUACUC A CCAUUAUG | 8632 | CATAATCC GGCTAGCTACAACGA CACTACAT | 19226 |
| 1456 | UACUCACC A UUAUGCAA | 8633 | TTCCATAA GGCTAGCTACAACGA CGTCAGTA | 19227 |
| 1459 | UCACCAUU A UCCAAGUG | 1701 | CACTTCCA GGCTAGCTACAACGA AATCCTCA | 19228 |
| 1465 | UUAUCCAA C UCAGUCAA | 8634 | TTCACTCA GGCTAGCTACAACGA TTCCATAA | 19229 |
| 1469 | CCAACUGA G UCAAACAC | 8635 | CTCTTTCA GGCTAGCTACAACGA TCACTTCC | 19230 |
| 1478 | UCAAAGAG A CACACGAA | 8636 | TTCCTCTG GGCTAGCTACAACGA CTCTTTCA | 19231 |
| 1480 | AAACACAC A CACGAAAU | 6327 | ATTTCCTG GGCTAGCTACAACGA GTCTCTTT | 19232 |
| 1487 | CACAGGAA A UUACACUG | 8637 | CAGTGTAA GGCTAGCTACAACGA TTCCTGTG | 19233 |
| 1490 | AGGAAAUU A CACUGUCA | 1703 | TGACAGTG GGCTAGCTACAACGA AATTTCCT | 19234 |
| 1492 | GAAAUUAC A CUGUCAUC | 6329 | GATOACAG GGCTAGCTACAACGA GTAATTTC | 19235 |
| 1495 | AUUACACU G UCAUCCUU | 8638 | AAGGATGA GGCTAGCTACAACGA AGTGTAAT | 19236 |
| 1498 | ACACUGUC A UCCUUACC | 6331 | GGTAAGGA GGCTAGCTACAACGA GACAGTGT | 19237 |
| 1504 | UCAUCCUU A CCAAUCCC | 1707 | GGGATTGG GGCTAGCTACAACGA AAGGATGA | 19238 |
| 1508 | CCUUACCA A UCCCAUUU | 8639 | AAATGGGA GGCTAGCTACAACGA TGGTAAGG | 19239 |
| 1513 | CCAAUCCC A UUUCAAAG | 6338 | CTTTGAAA GGCTAGCTACAACGA GGGATTGG | 19240 |
| 1527 | AAGGAGAA G CAGAGCCA | 8640 | TGGCTCTG GGCTAGCTACAACGA TTCTCCTT | 19241 |
| 1532 | GAAGCAGA G CCAUGUGG | 8641 | CCACATGG GGCTAGCTACAACGA TCTGCTTC | 19242 |
| 1535 | GCAGAGCC A UGUGGUCU | 6342 | AGACCACA GGCTAGCTACAACGA GGCTCTGC | 19243 |
| 1537 | AGAGCCAU G UGGUCUCU | 8642 | AGAGACCA GGCTAGCTACAACGA ATGGCTCT | 19244 |
| 1540 | GCCAUGUG U UCUCUCUG | 8643 | CAGAGAGA GGCTAGCTACAACGA CACATGGC | 19245 |
| 1549 | UCUCUCUG U UUGUGUAU | 8644 | ATACACAA GGCTAGCTACAACGA CAGAGAGA | 19246 |
| 1552 | CUCUGGUU C UGUAUGUC | 8645 | GACATACA GGCTAGCTACAACGA AACCAGAG | 19247 |
| 1554 | CUGGUUGU C UAUGUCCC | 8646 | GGGACATA GGCTAGCTACAACGA ACAACCAG | 19248 |
| 1556 | GGUUGUGU A UGUCCCAC | 1716 | GTGGGACA GGCTAGCTACAACGA ACACAACC | 19249 |
| 1558 | UUGUGUAU C UCCCACCC | 8647 | GGGTGGGA GGCTAGCTACAACGA ATACACAA | 19250 |
| 1563 | UAUGUCCC A CCCCAGAU | 6348 | ATCTGGGG GGCTAGCTACAACGA GGGACATA | 19251 |
| 1570 | CACCCCAG A UUGGUGAG | 8648 | CTCACCAA GGCTAGCTACAACGA CTGGGGTG | 19252 |
| 1574 | CCAGAUUG G UGAGAAAU | 8649 | ATTTCTCA GGCTAGCTACAACGA CAATCTGG | 19253 |
| 1581 | GGUGAGAA A UCUCUAAU | 8650 | ATTAGAGA GGCTAGCTACAACGA TTCTCACC | 19254 |
| 1588 | AAUCUCUA A UCUCUCCU | 8651 | AGGAGAGA GGCTAGCTACAACGA TAGAGATT | 19255 |
| 1597 | UCUCUCCU G UGGAUUCC | 8652 | GGAATCCA GGCTAGCTACAACGA AGGAGAGA | 19256 |
| 1601 | UCCUGUGG A UUCCUACC | 8653 | GGTAGGAA GGCTAGCTACAACGA CCACAGGA | 19257 |
| 1607 | GGAUUCCU A CCAGUACG | 1727 | CGTACTGG GGCTAGCTACAACGA AGGAATCC | 19258 |
| 1611 | UCCUACCA G UACGGCAC | 8654 | GTGCCGTA GGCTAGCTACAACGA TGGTAGGA | 19259 |
| 1613 | CUACCAGU A CGGCACCA | 1728 | TGGTGCCG GGCTAGCTACAACGA ACTGGTAG | 19260 |
| 1616 | CCAGUACG C CACCACUC | 8655 | GAGTGGTG GGCTAGCTACAACGA CGTACTGG | 19261 |
| 1618 | AGUACGGC A CCACUCAA | 6363 | TTGAGTGG GGCTAGCTACAACGA GCCGTACT | 19262 |
| 1621 | ACGGCACC A CUCAAACG | 6365 | CCTTTGAG GGCTAGCTACAACGA GGTGCCGT | 19263 |
| 1627 | CCACUCAA A CGCUGACA | 8656 | TGTCAGCG GGCTAGCTACAACGA TTGAGTGG | 19264 |
| 1629 | ACUCAAAC G CUGACAUG | 8657 | CATGTCAG GGCTAGCTACAACGA GTTTGAGT | 19265 |
| 1633 | AAACGCUG A CAUGUACG | 8658 | CGTACATG GGCTAGCTACAACGA CAGCGTTT | 19266 |
| 1635 | ACGCUGAC A UGUACGGU | 6369 | ACCGTACA GGCTAGCTACAACGA GTCAGCGT | 19267 |
| 1637 | GCUGACAU G UACGGUCU | 8659 | AGACCGTA GGCTAGCTACAACGA ATGTCAGC | 19268 |
| 1639 | UGACAUGU A CGGUCUAU | 1730 | ATAGACCG GGCTAGCTACAACGA ACATGTCA | 19269 |
| 1642 | CAUGUACG G UCUAUGCC | 8660 | GGCATAGA GGCTAGCTACAACGA CGTACATG | 19270 |
| 1646 | UACGGUCU A UGCCAUUC | 1732 | GAATGGCA GGCTAGCTACAACGA AGACCGTA | 19271 |
| 1648 | CGGUCUAU G CCAUUCCU | 8661 | AGGAATGG GGCTAGCTACAACGA ATAGACCG | 19272 |
| 1651 | UCUAUGCC A UUCCUCCC | 6372 | GGGAGGAA GGCTAGCTACAACGA GGCATAGA | 19273 |
| 1662 | CCUCCCCC G CAUCACAU | 8662 | ATGTGATG GGCTAGCTACAACGA GGGGGAGG | 19274 |
| 1664 | UCCCCCGC A UCACAUCC | 6379 | GGATGTGA GGCTAGCTACAACGA GCGGGGGA | 19275 |
| 1667 | CCCGCAUC A CAUCCACU | 6380 | AGTGGATG GGCTAGCTACAACGA GATGCGGG | 19276 |
| 1669 | CGCAUCAC A UCCACUGG | 6381 | CCAGTGGA GGCTAGCTACAACGA GTGATGCG | 19277 |
| 1673 | UCACAUCC A CUGGUAUU | 6383 | AATACCAG GGCTAGCTACAACGA GGATGTGA | 19278 |
| 1677 | AUCCACUG G UAUUGGCA | 8663 | TGCCAATA GGCTAGCTACAACGA CAGTGGAT | 19279 |
| 1679 | CCACUGGU A UUGGCAGU | 1738 | ACTGCCAA GGCTAGCTACAACGA ACCAGTGG | 19280 |
| 1683 | UGGUAUUG G CAGGUGGA | 8664 | TCCAACTG GGCTAGCTACAACGA CAATACCA | 19281 |
| 1686 | UAUUGCCA G UUGGAGGA | 8665 | TCCTCCAA GGCTACCTACAACGA TGCCAATA | 19282 |
| 1698 | GAGGAAGA G UGCGCCAA | 8666 | TTGGCGCA GGCTAGCTACAACGA TCTTCCTC | 19283 |
| 1700 | GCAAGAGU G CGCCAACG | 8667 | CGTTGGCG GGCTAGCTACAACGA ACTCTTCC | 19284 |
| 1702 | AAGAGUGC G CCAACGAG | 8668 | CTCGTTGG GGCTAGCTACAACGA GCACTCTT | 19285 |
| 1706 | GUGCGCCA A CGAGCCCA | 8669 | TGGGCTCG CGCTAGCTACAACGA TGGCGCAC | 19286 |
| 1710 | GCCAACGA G CCCAGCCA | 8670 | TGGCTGGG GCCTAGCTACAACGA TCGTTGGC | 19287 |
| 1715 | CGAGCCCA G CCAAGCUG | 8671 | CAGCTTGG GGCTAGCTACAACGA TGCGCTCG | 19288 |
| 1720 | CCAGCCAA G CUGUCUCA | 8672 | TOACACAG GGCTAGCTACAACGA TTGGCTGG | 19289 |
| 1723 | GCCAAGCU G UCUCAGUG | 8673 | CACTGAGA GGCTAGCTACAACGA AGCTTGGC | 19290 |
| 1729 | CUGUCUCA G UGACAAAC | 8674 | GTTGGTCA GGCTAGCTACAACGA TGAGACAC | 19291 |
| 1732 | UCUCAGUG A CAAACCCA | 8675 | TGGGTTTG GGCTAGCTACAACGA CACTUAGA | 19292 |
| 1736 | AGUGACAA A CCCAUACC | 8676 | GGTATGGG GGCTAGCTACAACGA TTGTCACT | 19293 |
| 1740 | ACAAACCC A UACCCUUG | 6399 | CAAGGGTA GGCTAGCTACAACGA GGGTTTGT | 19294 |
| 1742 | AAACCCAU A CCCUUGUG | 1743 | CACAAGGG GGCTAGCTACAACGA ATGGGTTT | 19295 |
| 1748 | AUACCCUU G UGAAGAAU | 8677 | ATTCTTCA GGCTAGCTACAACGA AAGGGTAT | 19296 |
| 1755 | UGUGAAGA A UGGAGAAG | 8678 | CTTCTCCA GGCTAGCTACAACGA TCTTCACA | 19297 |
| 1763 | AUGGAGAA G UGUGGAGG | 8679 | CCTCCACA GGCTAGCTACAACGA TTCTCCAT | 19298 |
| 1765 | GGAGAAGU G UGGAGGAC | 8680 | GTCCTCCA GGCTAGCTACAACGA ACTTCTCC | 19299 |
| 1772 | UGUGGAGG A CUUCCAGG | 8681 | CCTGGAAG GGCTAGCTACAACGA CCTCCACA | 19300 |
| 1787 | GGGAGGAA A UAAAAUUG | 8682 | CAATTTTA GGCTAGCTACAACGA TTCCTCCC | 19301 |
| 1792 | GAAAUAAA A UUGAAGUU | 8683 | AACTTCAA GGCTAGCTACAACGA TTTATTTC | 19302 |
| 1798 | AAAUUGAA G UUAAUAAA | 8684 | TTTATTAA GGCTAGCTACAACGA TTCAATTT | 19303 |
| 1802 | UGAAGUUA A UAAAAAUC | 8685 | GATTTTTA GGCTAGCTACAACGA TAACTTCA | 19304 |
| 1808 | UAAUAAAA A UCAAUUUG | 8686 | CAAATTGA GGCTAGCTACAACGA TTTTATTA | 19305 |
| 1812 | AAAAAUCA A UUUGCUCU | 8687 | AGAGCAAA GGCTAGCTACAACGA TGATTTTT | 19306 |
| 1816 | AUCAAUUU G CUCUAAUU | 8688 | AATTAGAG GGCTAGCTACAACGA AAATTGAT | 19307 |
| 1822 | UUGCUCUA A UUGAAGGA | 8689 | TCCTTCAA GGCTAGCTACAACGA TAGAGCAA | 19308 |
| 1835 | AGGAAAAA A CAAAACUG | 8690 | CAGTTTTG GGCTAGCTACAACGA TTTTTCCT | 19309 |
| 1840 | AAAACAAA A CUGUAAGU | 8691 | ACTTACAG GGCTAGCTACAACGA TTTGTTTT | 19310 |
| 1843 | ACAAAACU G UAAGUACC | 8692 | GGTACTTA GGCTAGCTACAACGA AGTTTTGT | 19311 |
| 1847 | AACUGUAA G UACCCUUG | 8693 | CAAGGGTA GGCTAGCTACAACGA TTACAGTT | 19312 |
| 1849 | CUGUAAGU A CCCUUGUU | 1759 | AACAAGGG GGCTAGCTACAACGA ACTTACAG | 19313 |
| 1855 | GUACCCUU G UUAUCCAA | 8694 | TTGGATAA GGCTAGCTACAACGA AAGGGTAC | 19314 |
| 1858 | CCCUUGUU A UCCAAGCG | 1762 | CGCTTGGA GGCTAGCTACAACGA AACAAGGG | 19315 |
| 1864 | UUAUCCAA G CGGCAAAU | 8695 | ATTTGCCG GGCTAGCTACAACGA TTGGATAA | 19316 |
| 1867 | UCCAAGCG G CAAAUGUG | 8696 | CACATTTG GGCTAGCTACAACGA CGCTTGGA | 19317 |
| 1871 | AGCGGCAA A UGUGUCAG | 8697 | CTGACACA GGCTAGCTACAACGA TTGCCGCT | 19318 |
| 1873 | CGGCAAAU G UGUCAGCU | 8698 | AGCTGACA GGCTAGCTACAACGA ATTTGCCG | 19319 |
| 1875 | GCAAAUGU G UCAGCUUU | 8699 | AAAGCTGA GGCTAGCTACAACGA ACATTTGC | 19320 |
| 1879 | AUGUGUCA G CUUUGUAC | 8700 | GTACAAAG GGCTAGCTACAACGA TGACACAT | 19321 |
| 1884 | UCAGCUUU G UACAAAUG | 8701 | CATTTGTA GGCTAGCTACAACGA AAAGCTGA | 19322 |
| 1886 | AGCUUUGU A CAAAUGUG | 1767 | CACATTTG GGCTAGCTACAACGA ACAAAGCT | 19323 |
| 1890 | UUGUACAA A UGUGAAGC | 8702 | GCTTCACA GGCTAGCTACAACGA TTGTACAA | 19324 |
| 1892 | GUACAAAU G UGAAGCGG | 8703 | CCGCTTCA GGCTAGCTACAACGA ATTTGTAC | 19325 |
| 1897 | AAUGUGAA G CGGUCAAC | 8704 | GTTGACCG GGCTAGCTACAACGA TTCACATT | 19326 |
| 1900 | GUGAAGCG G UCAACAAA | 8705 | TTTGTTGA GGCTAGCTACAACGA CGCTTCAC | 19327 |
| 1904 | AGCGGUCA A CAAAGUCG | 8706 | CGACTTTG GGCTAGCTACAACGA TGACCGCT | 19328 |
| 1909 | UCAACAAA G UCUGGAGA | 8707 | TCTCCCGA GGCTAGCTACAACGA TTTGTTGA | 19329 |
| 1927 | GAGAGAGG G UGAUCUCC | 8708 | GGAGATCA GGCTAGCTACAACGA CCTCTCTC | 19330 |
| 1930 | AGAGGGUG A UCUCCUUC | 8709 | GAAGGAGA GGCTAGCTACAACGA CACCCTCT | 19331 |
| 1940 | CUCCUUCC A CGUGACCA | 6426 | TGGTCACG GGCTAGCTACAACGA GGAACGAG | 19332 |
| 1942 | CCUUCCAC G UGACCAGG | 8710 | CCTGGTCA GGCTAGCTACAACGA GTGGAAGG | 19333 |
| 1945 | UCCACGUG A CCAGGGGU | 8711 | ACCCCTGG GGCTAGCTACAACGA CACGTGGA | 19334 |
| 1952 | GACCAGGG G UCCUGAAA | 8712 | TTTCAGGA GGCTAGCTACAACGA CCCTGGTC | 19335 |
| 1960 | GUCCUGAA A UUACUUUG | 8713 | CAAAGTAA GGCTAGCTACAACGA TTCAGGAC | 19336 |
| 1963 | CUGAAAUU A CUUUGCAA | 1776 | TTGCAAAG GGCTAGCTACAACGA AATTTCAG | 19337 |
| 1968 | AUUACUUU G CAACCUGA | 8714 | TCAGGTTG GGCTAGCTACAACGA AAAGTAAT | 19338 |
| 1971 | ACUUUGCA A CCUGACAU | 8715 | ATGTCAGG GGCTAGCTACAACGA TGCAAAGT | 19339 |
| 1976 | GCAACCUG A CAUGCAGC | 8716 | GCTGCATG GGCTAGCTACAACGA CAGGTTGC | 19340 |
| 1978 | AACCUGAC A UGCAGCCC | 6435 | GGGCTGCA GGCTAGCTACAACGA GTCAGGTT | 19341 |
| 1980 | CCUGACAU G CAGCCCAC | 8717 | GTGGGCTG GGCTAGCTACAACGA ATGTCAGG | 19342 |
| 1983 | GACAUCCA G CCCACUGA | 8718 | TCAGTGGG GGCTAGCTACAACGA TGCATGTC | 19343 |
| 1987 | UGCAGCCC A CUGAGCAG | 6439 | CTGCTCAG GGCTAGCTACAACGA GGGCTGCA | 19344 |
| 1992 | CCCACUGA G CAGGAGAG | 8719 | CTCTCCTG GGCTAGCTACAACGA TCAGTGGG | 19345 |
| 2000 | UCAGGAGA G CGUUUCUU | 8720 | AAGACACG GGCTAGCTACAACGA TCTCCTGC | 19346 |
| 2002 | AGGAGAUC G UGUCUUUG | 8721 | CAAAGACA GGCTAGCTACAACGA GCTCTCCT | 19347 |
| 2004 | GAGAGCGU G UCUUUGUG | 8722 | CACAAAGA GGCTAGCTACAACGA ACGCTCTC | 19348 |
| 2010 | GUGUCUUU G UGGUGCAC | 8723 | GTGCACCA GGCTAGCTACAACGA AAAGACAC | 19349 |
| 2013 | UCUUUGUG G UGCACUGC | 8724 | GCAGTGCA GGCTAGCTACAACGA CACAAAGA | 19350 |
| 2015 | UUUGUGGU G CACUGCAG | 8725 | CTGCAGTG GGCTAGCTACAACGA ACCACAAA | 19351 |
| 2017 | UGUGGUGC A CUCCAGAC | 6443 | GTCTGCAG GGCTAGCTACAACGA UCACCACA | 19352 |
| 2020 | GGUGCACU G CAGACAGA | 8726 | TCTGTCTG GGCTAGCTACAACGA AGTGCACC | 19353 |
| 2024 | CACUGCAG A CAGAUCUA | 8727 | TAGATCTG GGCTAGCTACAACGA CTGCAGTG | 19354 |
| 2028 | GCAGACAG A UCUACGUU | 8728 | AACGTAGA GGCTAGCTACAACGA CTGTCTGC | 19355 |
| 2032 | ACAGAUCU A CGUUUGAG | 1783 | CTCAAACG GGCTAGCTACAACGA AGATCTGT | 19356 |
| 2034 | AGAUCUAC G UUUGAGAA | 8729 | TTCTCAAA GGCTAGCTACAACGA GTAGATCT | 19357 |
| 2042 | GUUUGAGA A CCUCACAU | 8730 | ATGTGAGG GGCTAGCTACAACGA TCTCAAAC | 19358 |
| 2047 | AGAACCUC A CAUGGUAC | 6450 | GTACCATG GGCTAGCTACAACGA GAGGTTCT | 19359 |
| 2049 | AACCUCAC A UGGUACAA | 6451 | TTGTACCA GGCTAGCTACAACGA GTGAGGTT | 19360 |
| 2052 | CUCACAUG G UACAAGCU | 8731 | AGCTTGTA GGCTAGCTACAACGA CATGTGAG | 19361 |
| 2054 | CACAUGGU A CAAGCUUG | 1787 | CAAGCTTG GGCTAGCTACAACGA ACCATGTG | 19362 |
| 2058 | UGGUACAA G CUUGGCCC | 8732 | GGGCCAAG GGCTAGCTACAACGA TTGTACCA | 19363 |
| 2063 | CAAGCUUG G CCCACAGC | 8733 | GCTGTGGG GGCTAGCTACAACGA CAAGCTTG | 19364 |
| 2067 | CUUGGCCC A CAGCCUCU | 6456 | AGAGGCTG GGCTAGCTACAACGA GGGCCAAG | 19365 |
| 2070 | GGCCCACA G CCUCUGCC | 8734 | GGCAGAGG GGCTAGCTACAACGA TGTGGGCC | 19366 |
| 2076 | CAUCCUCU G CCAAUCCA | 8735 | TGGATTUG GGCTAGCTACAACGA AGAGGCTG | 19367 |
| 2080 | CUCUGCCA A UCCAUGUG | 8736 | CACATGGA GGCTAGCTACAACGA TGGCAUAG | 19368 |
| 2084 | GCCAAUCC A UGUGUGAG | 6464 | CTCCCACA GGCTAGCTACAACGA GGATTGGC | 19369 |
| 2086 | CAAUCCAU G UGGGAGAG | 8737 | CTCTCCCA GGCTAGCTACAACGA ATGGATTG | 19370 |
| 2094 | GUGGUAGA G UUGCCCAC | 8738 | GTGGGCAA GGCTAGCTACAACGA TCTCCCAC | 19371 |
| 2097 | GUAGAUUU G CCCACACC | 8739 | GGTGTGUG GGCTAGCTACAACGA AACTCTCC | 19372 |
| 2101 | AGUUGCCC A CACCUGUU | 6467 | AACAGUTG GGCTAGCTACAACGA GGGCAACT | 19373 |
| 2103 | UUGCCCAC A CCUUUUUU | 6468 | CAAACAGG GGCTAGCTACAACGA GTGGGCAA | 19374 |
| 2107 | CCACACCU G UUUGCAAG | 8740 | CTTGCAAA GGCTAGCTACAACGA AGGTGTGG | 19375 |
| 2111 | ACCUGUUU G CAAGAACU | 8741 | AGTTCTTU GGCTAGCTACAACGA AAACAUGT | 19376 |
| 2117 | UUGCAAUA A CUUGGAUA | 8742 | TATCCAAG GGCTAGCTACAACGA TCTTGCAA | 19377 |
| 2123 | GAACUUGG A UACUCUUU | 8743 | AAAGAGTA GGCTAGCTACAACGA CCAAUTTC | 19378 |
| 2125 | ACUUGGAU A CUCUUUGG | 1795 | CCAAAGAG GGCTAGCTACAACGA ATCCAAGT | 19379 |
| 2136 | CUUUGGAA A UUGAAUGC | 8744 | GCATTCAA GGCTAGCTACAACGA TTCCAAAG | 19380 |
| 2141 | GAAAUUGA A UGCCACCA | 8745 | TGGTGGCA GGCTAGCTACAACCA TCAATTTC | 19281 |
| 2143 | AAUUGAAU G CCACCAUG | 8746 | CATGGTGG GGCTAGCTACAACGA ATTCAATT | 19382 |
| 2146 | UGAAUGCC A CCAUGUUC | 6476 | GAACATGG GCCTAGCTACAACGA GGCATTCA | 19383 |
| 2149 | AUGCCACC A UGUUCUCU | 6478 | AGAGAACA GGCTAGCTACAACGA GGTGGCAT | 19384 |
| 2151 | GCCACCAU G UUCUCUAA | 8747 | TTAGAGAA GGCTAGCTACAACGA ATGGTGGC | 19385 |
| 2159 | GUUCUCUA A UAGCACAA | 8748 | TTGTGCTA GGCTAGCTACAACGA TAGAGAAC | 19386 |
| 2162 | CUCUAAUA G CACAAAUG | 8749 | CATTTGTG GGCTAGCTACAACGA TATTAGAG | 19387 |
| 2164 | CUAAUAGC A CAAAUGAC | 6481 | GTCATTTG GGCTAGCTACAACGA GCTATTAG | 19388 |
| 2168 | UAGCACAA A UGACAUUU | 8750 | AAATGTCA GGCTAGCTACAACGA TTGTGCTA | 19389 |
| 2171 | CACAAAUG A CAUUUUGA | 8751 | TCAAAATG GGCTAGCTACAACGA CATTTGTG | 19390 |
| 2173 | CAAAUGAC A UUUUGAUC | 6483 | GATCAAAA GGCTAGCTACAACGA GTCATTTG | 19391 |
| 2179 | ACAUUUUG A UCAUGGAG | 8752 | CTCCATGA GGCTAGCTACAACGA CAAAATGT | 19392 |
| 2182 | UUUUGAUC A UGGAGCUU | 6484 | AAGCTCCA GGCTAGCTACAACGA GATCAAAA | 19393 |
| 2187 | AUCAUGGA G CUUAAGAA | 8753 | TTCTTAAG GGCTAGCTACAACGA TCCATGAT | 19394 |
| 2195 | GCUUAAGA A UGCAUCCU | 8754 | AGGATGCA GGCTAGCTACAACGA TCTTAAGC | 19395 |
| 2197 | UUAAGAAU G CAUCCUUG | 8755 | CAAGGATG GGCTAGCTACAACGA ATTCTTAA | 19396 |
| 2199 | AAGAAUGC A UCCUUGCA | 6486 | TGCAAGGA GGCTAGCTACAACGA GCATTCTT | 19397 |
| 2205 | GCAUCCUU C CAGGACCA | 8756 | TGGTCCTG GGCTAGCTACAACGA AAGGATGC | 19398 |
| 2210 | CUUGCAGG A CCAAGGAG | 8757 | CTCCTTGG GGCTAGCTACAACGA CCTGCAAG | 19399 |
| 2219 | CCAAGGAG A CUAUGUCU | 8758 | AGACATAG GGCTAGCTACAACGA CTCCTTGG | 19400 |
| 2222 | AGGAGACU A UGUCUGCC | 1813 | GGCAGACA GGCTAGCTACAACGA AGTCTCCT | 19401 |
| 2224 | GAGACUAU G UCUGCCUU | 8759 | AAGGCAGA GGCTAGCTACAACGA ATAGTCTC | 19402 |
| 2228 | CUAUGUCU C CCUUGCUC | 8760 | GAGCAAGG GGCTAGCTACAACGA AGACATAG | 19403 |
| 2233 | UCUGCCUU G CUCAAGAC | 8761 | GTCTTGAG GGCTAGCTACAACGA AAGGCAGA | 19404 |
| 2240 | UGCUCAAG A CAGGAAGA | 8762 | TCTTCCTG GGCTAGCTACAACGA CTTGAGCA | 19405 |
| 2248 | ACAGGAAG A CCAAGAAA | 8763 | TTTCTTGG GGCTAGCTACAACGA CTTCCTGT | 19406 |
| 2259 | AAGAAAAG A CAUUGCGU | 8764 | ACGCAATG GGCTAGCTACAACGA CTTTTCTT | 19407 |
| 2261 | GAAAAGAC A UUGCGUGG | 6501 | CCACGCAA GGCTAGCTACPACGA GTCTTTTC | 19408 |
| 2264 | AAGACAUU G CGUGGUCA | 8765 | TGACCACG GGCTAGCTACAACGA AATGTCTT | 19409 |
| 2266 | GACAUUGC G UGGUCAGG | 8766 | CCTGACCA GGCTAGCTACAACGA GCAATGTC | 19410 |
| 2269 | AUUGCGUG G UCAGGCAG | 8767 | CTGCCTGA GGCTAGCTACAACGA CACGCAAT | 19411 |
| 2274 | GUGGUCAG G CACCUCAC | 8768 | GTGAGCTG GGCTAGCTACAACGA CTGACCAC | 19412 |
| 2277 | GUCAGGCA G CUCACAGU | 8769 | ACTGTGAG GGCTAGCTACAACGA TGCCTGAC | 19413 |
| 2281 | GGCAGCUC A CAGUCCUA | 6505 | TAGGACTG GGCTAGCTACAACGA GAGCTGCC | 19414 |
| 2284 | AGCUCACA C UCCUAGAG | 8770 | CTCTAGGA GGCTAGCTACAACGA TGTGAGCT | 19415 |
| 2292 | GUCCUAGA G CGUGUGGC | 8771 | GCCACACG GGCTAGCTACAACGA TCTAGGAC | 19416 |
| 2294 | CCUAGAGC G UGUGGCAC | 8772 | GTGCCACA GGCTAGCTACAACGA GCTCTAGG | 19417 |
| 2296 | UAGAGCGU G UGGCACCC | 8773 | GGGTGCCA GGCTAGCTACAACGA ACGCTCTA | 19418 |
| 2299 | AGCGUGUG G CACCCACG | 8774 | CGTGGGTG GGCTAGCTACAACGA CACACGCT | 19419 |
| 2301 | CGUGUGGC A CCCACGAU | 6509 | ATCGTGGG GGCTAGCTACAACGA GCCACACG | 19420 |
| 2305 | UGGCACCC A CGAUCACA | 6512 | TGTGATCC GGCTAGCTACAACGA GGGTGCCA | 19421 |
| 2308 | CACCCACG A UCACAGGA | 8775 | TCCTGTGA GGCTAGCTACAACGA CGTGGGTG | 19422 |
| 2311 | CCACGAUC A CAGGAAAC | 6513 | GTTTCCTG GGCTAGCTACAACGA GATCGTGG | 19423 |
| 2318 | CACAGGAA A CCUGGAGA | 8776 | TCTCCAGG GGCTAGCTACAACGA TTCCTGTG | 19424 |
| 2327 | CCUGGAGA A UCAGACGA | 8777 | TCGTCTGA GGCTAGCTACAACGA TCTCCAGG | 19425 |
| 2332 | AGAAUCAG A CGACAAGU | 8778 | ACTTGTCG GGCTAGCTACAACGA CTGATTCT | 19426 |
| 2335 | AUCAGACG A CAAGUAUU | 8779 | AATACTTG GGCTAGCTACAACGA CGTCTGAT | 19427 |
| 2339 | GACGACAA G UAUUGGGG | 8780 | CCCCAATA GGCTAGCTACAACGA TTGTCGTC | 19428 |
| 2341 | CGACAAGU A UUGGGGAA | 1824 | TTCCCCAA GGCTAGCTACAACGA ACTTGTCG | 19429 |
| 2351 | UGGGGAAA G CAUCGAAG | 8781 | CTTCGATG GGCTAGCTACAACGA TTTCCCCA | 19430 |
| 2353 | GGGAAAGC A UCGAAGUC | 6519 | GACTTCGA GGCTAGCTACAACGA GCTTTCCC | 19431 |
| 2359 | GCAUCGAA G UCUCAUGC | 8782 | CCATGAGA GGCTAGCTACAACGA TTCGATGC | 19432 |
| 2364 | GAAGUCUC A UGCACGGC | 6521 | GCCGTGCA GGCTAGCTACAACGA GAGACTTC | 19433 |
| 2366 | AGUCUCAU G CACGGCAU | 8783 | ATGCCGTG GGCTAGCTACAACGA ATGAGACT | 19434 |
| 2368 | UCUCAUGC A CCGCAUCU | 6522 | AGATGCCG GGCTAGCTACAACGA CCATCAGA | 19435 |
| 2371 | CAUGCACG G CAUCUGGG | 8784 | CCCAGATG GGCTAGCTACAACGA CGTGCATG | 19436 |
| 2373 | UGCACGGC A UCUGGGAA | 6523 | TTCCCAGA GGCTAGCTACAACCA GCCGTGCA | 19437 |
| 2381 | AUCUCGGA A UCCCCCUC | 8785 | GAGGGGGA GGCTAGCTACAACGA TCCCAGAT | 19438 |
| 2391 | CCCCCUCC A CAGATCAU | 6531 | ATGATCTG GGCTAGCTACAACGA GGAGGGGG | 19439 |
| 2395 | CUCCACAG A UCAUGUGG | 8786 | CCACATGA GGCTAGCTACAACGA CTGTGGAG | 19440 |
| 2398 | CACAGAUC A UGUGGUUU | 6533 | AAACCACA GGCTAGCTACAACGA GATCTGTG | 19441 |
| 2400 | CAGAUCAU 0 UGGUUUAA | 8787 | TTAAACCA GGCTAGCTACAACGA ATGATCTG | 19442 |
| 2403 | AUCAUGUG G UUUAAAGA | 8788 | TCTTTAAA GGCTAGCTACAACGA CACATGAT | 19443 |
| 2411 | GUUTAAAG A UAAUGAGA | 8789 | TCTCATTA GGCTAGCTACAACGA CTTTAAAC | 19444 |
| 2414 | UAAAGAUA A UGAGACCC | 8790 | GGGTCTCA GGCTAGCTACAACGA TATCTTTA | 19445 |
| 2419 | AUAAUGAG A CCCUUGUA | 8791 | TACAAGGG GGCTAGCTACAACGA CTCATTAT | 19446 |
| 2425 | AGACCCUU G UAGAAGAC | 8792 | GTCTTCTA GGCTAGCTACAACGA AAGGGTCT | 19447 |
| 2432 | UGUAGAAG A CUCAGGCA | 8793 | TGCCTGAG GGCTAGCTACAACGA CTTCTACA | 19448 |
| 2438 | AGACUCAG G CAUUGUAU | 8794 | ATACAATG GGCTAGCTACAACGA CTGAGTCT | 19449 |
| 2440 | ACUCAGGC A UUGUAUUG | 6539 | CAATACAA GGCTAGCTACAACGA GCCTGAGT | 19450 |
| 2443 | CAGGCAUU C UAUUGAAG | 8795 | CTTCAATA GGCTAGCTACAACGA AATGCCTG | 19451 |
| 2445 | GGCAUUGU A UUGAAGGA | 1841 | TCCTTCAA GGCTAGCTACAACGA ACAATGCC | 19452 |
| 2453 | AUUGAAGG A UGGGAACC | 8796 | GGTTCCCA GGCTAGCTACAACGA CCTTCAAT | 19453 |
| 2459 | GGAUGCGA A CCGGAACC | 8797 | GGTTCCGG GCCTAGCTACAACGA TCCCATCC | 19454 |
| 2465 | GAACCGGA A CCUCACUA | 8798 | TAGTGAGG GGCTAGCTACAACGA TCCCGTTC | 19455 |
| 2470 | GGAACCUC A CUAUCCGC | 6543 | UCOGATAG GGCTAGCTACAACGA GAGGTTCC | 19456 |
| 2473 | ACCUCACU A UCCUCAGA | 1844 | TCTGCGGA GGCTAGCTACAACGA AGTGAGGT | 19457 |
| 2477 | CACUAUCC G CAGAGUGA | 8799 | TCACTCTG GGCTAGCTACAACGA GGATAGTG | 19458 |
| 2482 | UCCUCAGA G UGAGGAAG | 8800 | CTTCCTCA GGCTAGCTACAACGA TCTGCGGA | 19459 |
| 2495 | GAAGGAGG A CGAAGGCC | 8801 | GGCCTTCG GGCTAGCTACAACGA CCTCCTTC | 19460 |
| 2501 | GGACGAAG G CCUCUACA | 8802 | TGTAGAGG GGCTAGCTACAACGA CTTCGTCC | 19461 |
| 2507 | AGGCCUCU A CACCUGCC | 1847 | GGCAGGTG GGCTAGCTACAACGA AGAGGCCT | 19462 |
| 2509 | GCCUCUAC A CCUGCCAG | 6550 | CTGGCAGG GGCTAGCTACAACGA GTAGAGGC | 19463 |
| 2513 | CUACACCU G CCAUGCAU | 8803 | ATGCCTGG GGCTAGCTACAACGA AGGTGTAG | 19464 |
| 2518 | CCUGCCAG G CAUGCAGU | 8804 | ACTGCATG GGCTAGCTACAACGA CTGGCAGG | 19465 |
| 2520 | UGCCAGGC A UGCAGUGU | 6555 | ACACTUCA GGCTAGCTACAACGA GCCTGGCA | 19466 |
| 2522 | CCAGGCAU G CAGUGUUC | 8805 | GAACACTG GGCTAGCTACAACGA ATGCCTGG | 19467 |
| 2525 | GUCAUCCA G UGUUCUUG | 8806 | CAAGAACA GGCTAGCTACAACGA TGCATGCC | 19468 |
| 2527 | CAUGCAGU G UUCUUGGC | 8807 | GCCAAGAA GGCTAGCTACAACGA ACTGCATG | 19469 |
| 2534 | UGUUCTUG G CUGUUCAA | 8808 | TTGCACAG GGCTAGCTACAACGA CAAGAACA | 19470 |
| 2537 | UCUUGUCU G UGCAAAAG | 8809 | CTTTTGCA GGCTAGCTACAACGA AGCCAAGA | 19471 |
| 2539 | UUGGCUGU G CAAAAUUG | 8810 | CACTTTTG GGCTAGCTACAACGA ACAGCCAA | 19472 |
| 2545 | GUGCAAAA G UGGAUGCA | 8811 | TGCCTCCA GGCTAGCTACAACGA TTTTGCAC | 19473 |
| 2551 | AAGUUGAG G CAUUUUUC | 8812 | GAAAAATU GGCTAGCTACAACGA CTCCACTT | 19474 |
| 2553 | GUGUAUGC A UUUUUCAU | 6560 | ATGAAAAA GGCTAGCTACAACGA GCCTCCAC | 19475 |
| 2560 | CAGUUGUC A UAAUAGAU | 6561 | TTCTATTA GGCTAGCTACAACGA GAAAAATG | 19476 |
| 2563 | UUUUCAUA A UAGAAGGU | 8813 | ACCTTCTA GGCTAGCTACAACGA TATGAAAA | 19477 |
| 2570 | AAUAGAAG G UGCCCAGG | 8814 | CCTGGGCA GGCTAGCTACAACGA CTTCTATT | 19478 |
| 2572 | UAGAAGGU G CCCAGGAA | 8815 | TTCCTGGG GGCTAGCTACAACGA ACCTTCTA | 19479 |
| 2584 | AGGAAAAG A CGAACUUG | 8818 | CAAGTTCG GGCTAGCTACAACGA CTTTTCCT | 19480 |
| 2588 | AAAGACGA A CUUCGAAA | 8817 | TTTCCAAG GGCTAGCTACAACGA TCGTCTTT | 19481 |
| 2596 | ACUUGGAA A UCAUUAUU | 8818 | AATAATGA GGCTAGCTACAACGA TTCCAAGT | 19482 |
| 2599 | UGGAAAUC A UUAUUCUA | 6566 | TAGAATAA GGCTAGCTACAACGA CATTTCCA | 19483 |
| 2602 | AAAUCAUU A UUCUAGUA | 1861 | TACTAGAA GGCTAGCTACAACGA AATGATTT | 19484 |
| 2608 | UUAUUCUA C UAGGCACG | 8819 | CGTGCCTA GGCTAGCTACAACGA TAGAATAA | 19485 |
| 2612 | UCUAGUAG C CACGGCGG | 8820 | CCGCCGTG GGCTAGCTACAACGA CTACTAGA | 19486 |
| 2614 | UAGUAGCC A CGGCGGUG | 8821 | CACCCCCC GGCTAGCTACAACGA CCCTACTA | 19487 |
| 2617 | UAGGCACG C CGCUGAUU | 8822 | AATCACCC GGCTAGCTACAACGA CGTGCCTA | 19488 |
| 2620 | GCACGCCG G UCAUUGCC | 8823 | GCCAATCA GGCTAGCTACAACGA CGCCGTGC | 19489 |
| 2623 | CGCCGCUC A UUGCCAUG | 8824 | CATGGCAA GGCTAGCTACAACGA CACCCCCC | 19490 |
| 2626 | CGGUGAUU G CCAUGUUC | 8825 | GAACATGC GGCTAGCTACAACGA AATCACCG | 19491 |
| 2629 | UGAUUCCC A UCUUCUUC | 6570 | CAAGAACA GGCTAGCTACAACGA GGCAATCA | 19492 |
| 2631 | AUUCCCAU G UUCUUCUG | 8826 | CAGAAGAA GGCTAGCTACAACGA ATGGCAAT | 19493 |
| 2640 | UUCUUCUG G CUACUUCU | 8827 | AGAACTAG GGCTAGCTACAACGA CACAAGAA | 19494 |
| 2643 | UUCUGGCU A CUUCUUCU | 1871 | ACAACAAC GGCTAGCTACAACGA ACCCAGAA | 19495 |
| 2650 | UACUUCUU G UCAUCAUC | 8828 | CATCATGA GGCTAGCTACAACGA AAGAAGTA | 19496 |
| 2653 | CUCCUCUC A UCAUCCUA | 6576 | TAGGATCA GGCTAGCTACAACGA GACAAGAA | 19497 |
| 2656 | UUGUCAUC A UCCUACOC | 8829 | CCGTACCA GGCTAGCTACAACGA CATCACAA | 19498 |
| 2661 | AUCAUCCU A CGGACCGU | 8830 | ACCGTCCC GGCTAGCTACAACGA ACGATCAT | 19499 |
| 2665 | UCCUACGC A CCGUUAAG | 8831 | CTTAACCG GGCTAGCTACAACGA CCCTACCA | 19500 |
| 2668 | UACGCACC C UUAACCGC | 8832 | CCGCTTAA GGCTAGCTACAACGA GCTCCGTA | 19501 |
| 2673 | ACCGUUAA G CCGGCCAA | 8833 | TTGGCCCC GGCTAGCTACAACGA TTAACGGT | 19502 |
| 2677 | UUAAGCGG G CCAAUGCA | 8834 | TCCATTGG GGCTAGCTACAACGA CCCCTTAA | 19503 |
| 2681 | GCCCGCCA A UGCACGGC | 8835 | CCCCTCCA GGCTAGCTACAACGA TGCCCCGC | 19504 |
| 2691 | CGACGCCA A CUGAAGAC | 8836 | CTCTTCAC GGCTAGCTACAACGA TCCCCTCC | 19505 |
| 2698 | AACUCAAC A CACGCUAC | 8837 | GTACCCTC GGCTAGCTACAACGA CTTCAGTT | 19506 |
| 2702 | GAACACAC G CUACUUCU | 8838 | ACAAGTAC GGCTAGCTACAACGA CTCTCTTC | 19507 |
| 2705 | CACACCCU A CUUCUCCA | 1881 | TCCACAAC GGCTAGCTACAACGA ACCCTCTC | 19508 |
| 2709 | CCCUACUU G UCCAUCCU | 8839 | ACCATCCA GGCTAGCTACAACGA AACTACCC | 19509 |
| 2713 | ACUUCUCC A UCCUCAUC | 6588 | CATCACCA GGCTAGCTACAACGA CCACAACT | 19510 |
| 2716 | UCUCCAUC G UCAUCCAU | 8840 | ATCCATCA GGCTAGCTACAACGA CATCCACA | 19511 |
| 2719 | CCAUCCUC A UCCAUCCA | 6589 | TCCATCCA GGCTAGCTACAACGA CACCATCC | 19512 |
| 2723 | CCUCAUCC A UCCACAUC | 8841 | CATCTCCA GGCTAGCTACAACGA CCATCACC | 19513 |
| 2729 | CCAUCCAC A UCAACUCC | 8842 | CCACTTCA GGCTAGCTACAACGA CTCCATCC | 19514 |
| 2733 | CCACAUCA A CUCCCAUU | 8843 | AATCCCAC GGCTAGCTACAACGA TCATCTCC | 19515 |
| 2739 | CAACUCCC A UUCCAUGA | 6595 | TCATCCAA GGCTAGCTACAACGA CCCACTTC | 19516 |
| 2744 | CCCAUUCC A UCAACAUU | 8844 | AATCTTCA GGCTAGCTACAACGA CCAATCCC | 19517 |
| 2748 | UUCCAUCA A CAUUCUCA | 8845 | TCACAATC GGCTAGCTACAACGA TCATCCAA | 19518 |
| 2750 | CCAUCAAC A UUGUCAAC | 6596 | CTTCACAA GGCTAGCTACAACGA CTTCATCC | 19519 |
| 2753 | UCAACAUU G UCAACCAC | 8846 | CTCCTTCA GGCTAGCTACAACGA AATCTTCA | 19520 |
| 2757 | CAUUCUCA A CCACUCCC | 8847 | CCCACTCC GGCTAGCTACAACGA TCACAATC | 19521 |
| 2760 | UCUCAACC A CUCCCUUA | 8848 | TAACCCAC GGCTAGCTACAACGA CCTTCACA | 19522 |
| 2763 | CAACCACU G CCUUAUCA | 8849 | TCATAACC GGCTAGCTACAACGA ACTCCTTC | 19523 |
| 2768 | ACUCCCUU A UCAUCCCA | 1891 | TCCCATCA GGCTAGCTACAACGA AACCCACT | 19524 |
| 2771 | CCCUUAUC A UCCCACCA | 8850 | TCCTCCCA GGCTAGCTACAACGA CATAACCC | 19525 |
| 2773 | CUUAUCAU G CCACCAAA | 8851 | TTTCCTCC GGCTAGCTACAACGA ATCATAAC | 19526 |
| 2777 | UGAUCCCA G CAAAUGCG | 8852 | CCCATTTG GGCTAGCTACAACGA TCGCATCA | 19527 |
| 2781 | GCCAGCAA A UGGGAAUU | 8853 | AATTCCCA GGCTAGCTACAACGA TTGCTGGC | 19528 |
| 2787 | AAAUGGGA A UUCCCCAG | 8854 | CTGGGGAA GGCTAGCTACAACGA TCCCATTT | 19529 |
| 2798 | CCCCAGAG A CCGGCUGA | 8855 | TCAGCCGG GGCTAGCTACAACGA CTCTGGGG | 19530 |
| 2802 | AGAGACCG G CUGAAGCU | 8856 | AGCTTCAG GGCTAGCTACAACGA CGGTCTCT | 19531 |
| 2808 | CGGCUGAA G CUAGGUAA | 8857 | TTACCTAG GGCTAGCTACAACGA TTCACCCG | 19532 |
| 2813 | GAAGCUAG G UAAGCCUC | 8858 | GAGGCTTA GGCTAGCTACAACGA CTAGCTTC | 19533 |
| 2817 | CUAGGUAA G CCUCUUGG | 8859 | CCAAGAGG GGCTAGCTACAACGA TTACCTAG | 19534 |
| 2825 | GCCUCUGG G CCGUGGUG | 8860 | CACCACUG GGCTAGCTACAACGA CAAGAGGC | 19535 |
| 2828 | UCUUGGCC G UGGUGCCU | 8861 | AGGCACCA GGCTAGCTACAACGA GGCCAAGA | 19536 |
| 2831 | UGGCCGUG G UUCCUUUG | 8862 | CAAAGGCA GGCTAGCTACAACGA CACGGCCA | 19537 |
| 2833 | GCCCUGGU G CCUUUGGC | 8863 | GCCAAAGG GGCTAGCTACAACGA ACCACGGC | 19538 |
| 2840 | UGCCUUUG G CCAAGUGA | 8864 | TCACTTGG GGCTAGCTACAACGA CAAAGGCA | 19539 |
| 2845 | UUGGCCAA G UGAUUGAA | 8865 | TTCAATCA GGCTAGCTACAACGA TTGGCCAA | 19540 |
| 2848 | GCCAAGUG A UUGAAGCA | 8866 | TGCTTCAA GGCTAGCTACAACGA CACTTGGC | 19541 |
| 2854 | UGAUUGAU G CAGAUGCC | 8867 | GGCATCTG GGCTAGCTACAACGA TTCAATCA | 19542 |
| 2858 | UGAAGCAG A UGCCUUUG | 8868 | CAAAGGCA GGCTAGCTACAACGA CTGCTTCA | 19543 |
| 2860 | AAGCAGAU G CCUUUGGA | 8869 | TCCAAAGG GGCTAGCTACAACGA ATCTGCTT | 19544 |
| 2869 | CCUUUGGA A UUGACAAG | 8870 | CTTGTCAA GGCTAGCTACAACGA TCCAAAGG | 19545 |
| 2873 | UGGAAUUG A CAAGACAG | 8871 | CTGTCTTG GGCTAGCTACAACGA CAATTCCA | 19546 |
| 2878 | UUGACAAG A CAGCAACU | 8872 | AGTTGCTG GGCTAGCTACAACGA CTTGTCAA | 19547 |
| 2881 | ACAAGACA G CAACUUGC | 8873 | GCAAGTTG GGCTAGCTACAACGA TGTCTTGT | 19548 |
| 2884 | AGACAGCA A COUCCAUG | 8874 | CCTGCAAG GGCTAGCTACAACGA TGCTGTCT | 19549 |
| 2888 | AGCAACUU G CAGGACAG | 8875 | CTGTCCTG GGCTAGCTACAACGA AAGTTGCT | 19550 |
| 2893 | CUUGCAGG A CAGUAGCA | 8876 | TGCTACTG GGCTAGCTACAACGA CCTGCAAG | 19551 |
| 2896 | GCAGGACA G UAGCAGUC | 8877 | GACTGCTA GGCTAGCTACAACGA TGTGCTGC | 19552 |
| 2899 | GGACAGUA G CAGUCAAA | 8878 | TTTGACTG GGCTAGCTACAACGA TACTGTCC | 19553 |
| 2902 | CAGUAGCA G UCAAAAUG | 8879 | CATTTTGA GGCTAGCTACAACGA TGCTACTG | 19554 |
| 2908 | CAGUCAAA A UGUUGAAA | 8880 | TTTCAACA GGCTAGCTACAACGA TTTGACTG | 19555 |
| 2910 | GUCAAAAU G UUGAAAGA | 8881 | TCTTTCAA GGCTAGCTACAACGA ATTTTGAC | 19556 |
| 2923 | AAGAAGGA G CAACACAC | 8882 | GTGTGTTG GGCTAGCTACAACGA TCCTTCTT | 19557 |
| 2926 | AAGGAGCA A CACACAGU | 8883 | ACTGTGTG GGCTAGCTACAACGA TGCTCCTT | 19558 |
| 2928 | GGAGCAAC A CACAGUGA | 6631 | TCACTGTG GGCTAGCTACAACGA GTTGCTCC | 19559 |
| 2930 | AGCAACAC A CAGUGAGC | 6632 | GCTCACTG GGCTAGCTACAACGA GTGTTGCT | 19560 |
| 2933 | AACACACA G UGAUCAUC | 8884 | GATGCTCA GGCTAGCTACAACGA TGTGTGTT | 19561 |
| 2937 | CACAGUGA G CAUC0AGC | 8885 | GCTCGATG GGCTAGCTACAACGA TCACTGTG | 19562 |
| 2939 | CAGUGAGC A UCGAGCUC | 6634 | GAGCTCGA GGCTAGCTACAACGA GCTCACTG | 19563 |
| 2944 | AGCAUCGA G CUCUCAUG | 8886 | CATGAGAG GGCTAGCTACAACGA TCGATGCT | 19564 |
| 2950 | GAUCUCUC A UGUCUGAA | 6637 | TTCAGACA GGCTAGCTACAACGA GAGAGCTC | 19565 |
| 2952 | GCUCUCAU G UCUGAACU | 8887 | AGTTCAGA GGCTAGCTACAACGA ATGAGAGC | 19566 |
| 2958 | AUGUCUGA A CUCAAGAU | 8888 | ATCTTGAG GGCTAGCTACAACGA TCAGACAT | 19567 |
| 2965 | AACUCAAG A UCCUCAUU | 8889 | AATGAGGA GGCTAGCTACAACGA CTTGAGTT | 19568 |
| 2971 | AGAUCCUC A UUCAUAUU | 6643 | AATATGAA GGCTAGCTACAACGA GAGGATCT | 19569 |
| 2975 | CCUCAUUC A UAUUGGUC | 6644 | GACCAATA GGCTAGCTACAACGA GAATGAGG | 19570 |
| 2977 | UCAUUCAU A UUGGUCAC | 1917 | GTGACCAA GGCTAGCTACAACGA ATGAATGA | 19571 |
| 2981 | UCAUAUUG G UCACCAUC | 8890 | GATGGTGA GGCTAGCTACAACGA CAATATGA | 19572 |
| 2984 | UAUUGGUC A CCAUCUCA | 6645 | TGAGATGG GGCTAGCTACAACGA GACCAATA | 19573 |
| 2987 | UGGUCACC A UCUCAAUG | 6647 | CATTGAGA GGCTAGCTACAACGA GGTGACCA | 19574 |
| 2993 | CCAUCUCA A UGUGGUCA | 8891 | TGACCACA GGCTAGCTACAACGA TGAGATGG | 19575 |
| 2995 | AUCUCAAU C UGGUCAAC | 8892 | GTTGACCA GGCTAGCTACAACGA ATTGAGAT | 19576 |
| 2998 | UCAAUGUC G UCAACCUU | 8893 | AACGTTGA GGCTAGCTACAACGA CACATTGA | 19577 |
| 3002 | UGUGGUCA A CCUUCUAG | 8894 | CTAGAAGG GGCTAGCTACAACGA TGACCACA | 19578 |
| 3011 | CCUUCUAG G UGCCUGUA | 8895 | TACAGUCA GGCTAGCTACAACGA CTAGAAGG | 19579 |
| 3013 | UUCUAGGU C CCUGUACC | 8896 | GCTACAGG GGCTAGCTACAACGA ACCTAGAA | 19580 |
| 3017 | AGGUGCCU G UACCAAGC | 8897 | GCTTGCTA GCCTAGCTACAACGA AGGCACCT | 19581 |
| 3019 | GUGCCU3U A CCAAGCCA | 1926 | TGGCTTGG GGCTAGCTACAACGA ACAGGCAC | 19582 |
| 3024 | UGUACCAA G CCAGCAGG | 8898 | CCTCCTGG GCCTAGCTACAACGA TTGGTACA | 19583 |
| 3033 | CCAGGAGG G CCACUCAU | 8899 | ATGAGTGG GGCTAGCTACAACGA CCTCCTGG | 19584 |
| 3036 | CGAGGGCC A CUCAUGGU | 6661 | ACCATCAG GGCTAGCTACAACGA GGCCCTCC | 19585 |
| 3040 | GGCCACUC A UGGUGAUU | 6663 | AATCACCA GGCTAGCTACAACGA GAGTCGCC | 19586 |
| 3043 | CACUCAUG G UGAUUGUG | 8900 | CACAATCA GGCTAGCTACAACGA CATGAGTG | 19587 |
| 3046 | UCAUGGUG A UUGUGGAA | 8901 | TTCCACAA GGCTAGCTACAACGA CACCATGA | 19588 |
| 3049 | UGGUGAUU C UGGAAUUC | 8902 | CAATTCCA GCCTAGCTACAACGA AATCACCA | 19589 |
| 3054 | AUUGUGGA A UUCUGCAA | 8903 | TTCCAGAA GGCTAGCTACAACGA TCCACAAT | 19590 |
| 3059 | GGAAUUCU C CAAAUUUG | 8904 | CAAATTTG GGCTAGCTACAACGA AGAATTCC | 19591 |
| 3063 | UUCUGCAA A UUUCGAAA | 8905 | TTTCCAAA GGCTAGCTACAACGA TTGCACAA | 19592 |
| 3071 | AUUUGGAA A CCUGUCCA | 8906 | TGGACACG GCCTAGCTACAACGA TTCCAAAT | 19593 |
| 3075 | GGAAACCU C UCCACUUA | 8907 | TAACTGCA GGCTACCTACAACGA AGCTTTCC | 19594 |
| 3079 | ACCUGUCC A CUUACCUG | 6669 | CAGGTAAG GGCTAGCTACAACGA CGACAGCT | 19595 |
| 3083 | GUCCACUU A CCUGAGGA | 1935 | TCCTCAGG GGCTAGCTACAACGA AAGTGCAC | 19596 |
| 3092 | CCUGACGA C CAAGAGAA | 8908 | TTCTCTTC GGCTAGCTACAACGA TCCTCAGG | 19597 |
| 3101 | CAACAGAA A UCAAUUUC | 8909 | CAAATTCA GGCTAGCTACAACGA TTCTCTTC | 19598 |
| 3105 | AGAAAUGA A UUUCUCCC | 8910 | GGGACAAA GGCTAGCTACAACGA TCATTTCT | 19599 |
| 3109 | AUGAAUUU G UCCCCUAC | 8911 | CTAGGCGA GGCTAGCTACAACGA AAATTCAT | 19600 |
| 3116 | UCUCCCCU A CAACACCA | 1939 | TCCTCTTG GGCTAGCTACAACGA ACCCCACA | 19601 |
| 3121 | CCUACAAC A CCAAAGGG | 8912 | CCCTTTCC GGCTAGCTACAACGA CTTCTACG | 19602 |
| 3130 | CCAAACGC C CACCAUUC | 8913 | CAATCGTG GGCTAGCTACAACGA CCCTTTGC | 19603 |
| 3132 | AAAGGCCC A CCAUUCCG | 6681 | CGGAATCG GGCTAGCTACAACGA GCCCCTTT | 19604 |
| 3135 | GCCGCACG A UUCCGUCA | 8914 | TGACGGAA GGCTAGCTACAACGA CCTGCCCC | 19605 |
| 3140 | ACGAUUCC G UCAAGGGA | 8915 | TCCCTTCA GGCTAGCTACAACGA GCAATCGT | 19606 |
| 3152 | ACCGAAUG A CUACGUUG | 8916 | CAACGTAC GGCTAGCTACAACGA CTTTCCCT | 19607 |
| 3155 | GAAACACU A CGUUGCAG | 1943 | CTCCAACG GGCTAGCTACAACGA ACTCTTTC | 19608 |
| 3157 | AAGACUAC C UUGGAGCA | 8917 | TGCTCCAA GGCTAGCTACAACGA CTAGTCTT | 19609 |
| 3163 | ACCUUCGA C CAAUCCCU | 8918 | AGGGATTC GGCTAGCTACAACGA TCCAACGT | 19610 |
| 3166 | UUGGACCA A UCCCUCUC | 8919 | CACACCGA GGCTAGCTACAACGA TGCTCCAA | 19611 |
| 3172 | CAAUCCCU C UGGAUCUC | 8920 | CACATCCA GGCTAGCTACAACGA ACCGATTG | 19612 |
| 3176 | CCCUCUCG A UCUCAAAC | 8921 | GTTTCACA GGCTAGCTACAACGA CCACAGCG | 19613 |
| 3183 | CAUCUCAA A CCCCCCUU | 8922 | AACCCCCC GGCTAGCTACAACGA TTCACATC | 19614 |
| 3186 | CUCAAACC C CCCUUCCA | 8923 | TCCAACCC GGCTAGCTACAACGA CCTTTCAC | 19615 |
| 3188 | CAAACCCC C CUUCCACA | 8924 | TCTCCAAC GGCTAGCTACAACGA CCCCTTTC | 19616 |
| 3194 | CCCCUUGG A CACCAUCA | 8925 | TCATCCTC GGCTAGCTACAACGA CCAAGCCC | 19617 |
| 3197 | CUUCCACA C CAUCACCA | 8926 | TCGTCATC GGCTAGCTACAACGA TCTCCAAC | 19618 |
| 3199 | UCCACACC A UCACCACU | 6692 | ACTCCTCA GGCTAGCTACAACGA CCTCTCCA | 19619 |
| 3202 | ACACCAUC A CCACUACC | 6693 | GCTACTCC GGCTAGCTACAACGA CATCCTCT | 19620 |
| 3206 | CAUCACCA C UACCCACA | 8927 | TCTCGCTA GGCTAGCTACAACGA TCCTCATC | 19621 |
| 3209 | CACCACUA C CCACACCU | 8928 | ACCTCTCC GGCTAGCTACAACGA TACTCCTC | 19622 |
| 3215 | UACCCACA C CUCACCCA | 8929 | TCGCTCAC GGCTAGCTACAACGA TCTCCCTA | 19623 |
| 3220 | ACACCUCA C CCACCUCU | 8930 | ACACCTCC GGCTAGCTACAACGA TCACCTCT | 19624 |
| 3224 | CUCAGCCA G CUCUGGAU | 8931 | ATCCAGAG GGCTAGCTACAACGA TGGCTGAG | 19625 |
| 3231 | AGCUCUGG A UUUGUGGA | 8932 | TCCACAAA GGCTAGCTACAACGA CCAGAGCT | 19626 |
| 3235 | CUGGAUUU G UCCAGGAG | 8933 | CTCCTCCA GGCTAGCTACAACGA AAATCCAG | 19627 |
| 3246 | GAGGAGAA G UCCCUCAG | 8934 | CTGAGGGA GGCTAGCTACAACGA TTCTCCTC | 19628 |
| 3254 | GUCCCUCA G UGAUGUAG | 8935 | CTACATCA GGCTAGCTACAACGA TGAGGGAC | 19629 |
| 3257 | CCUCAGUG A UGUAGAAG | 8936 | CTTCTACA GGCTAGCTACAACGA CACTGAGG | 19630 |
| 3259 | UCAGUGAG G UAGAAGAA | 8937 | TTCTTCTA GGCTAGCTACAACGA ATCACTGA | 19631 |
| 3274 | AAGAGGAA G CUCCUGAA | 8938 | TTCAGGAG GGCTAGCTACAACGA TTCCTCTT | 19632 |
| 3284 | UCCUGAAC A UCUGUAUA | 8939 | TATACAGA GGCTAGCTACAACGA CTTCAGGA | 19633 |
| 3288 | GAAGAUCU G UAUAAGGA | 8940 | TCCTTATA GGCTAGCTACAACGA AGATCTTC | 19634 |
| 3290 | AGAUCUGU A UAAGGACU | 1959 | AGTCCTTA GGCTAGCTACAACGA ACAGATCT | 19635 |
| 3296 | GUAUAAGG A CUUCCUGA | 8941 | TCAGGAAG GGCTAGCTACAACGA CCTTATAC | 19636 |
| 3304 | ACUUCCUG A CCUUGGAG | 8942 | CTCCAAGG GGCTAGCTACAACGA CAGGAAGT | 19637 |
| 3312 | ACCUUGUA G CAUCUCAU | 8943 | ATGAGATG GGCTAGCTACAACGA TCCAAGGT | 19638 |
| 3314 | CUGUGAUC A UCUCAUCU | 6717 | AGATGAGA GGCTAGCTACAACGA GCTCCAAG | 19639 |
| 3319 | AGCAUCUC A UCUGUGAC | 6719 | GTAACAGA GGCTAGCTACAACGA GAGATGCT | 19640 |
| 3323 | UCUCAUCU G UUACAGCU | 8944 | AGCTUTAA GGCTAGCTACAACGA AGATGAGA | 19641 |
| 3326 | CAUCUUUU A CAGCUUCC | 1968 | GGAAGCTG GGCTAGCTACAACGA AACAGATG | 19642 |
| 3329 | CUGUUACA G CUUCCAAU | 8945 | CTTGGAAG GGCTAGCTACAACGA TGTAACAG | 19643 |
| 3337 | UCUUCCAA G UGGCUAAG | 8946 | CTTAGCCA GGCTAGCTACAACGA TTGGAAGC | 19644 |
| 3340 | UCCAAGUG G CUAAGUGC | 8947 | GCCCTTAG GGCTAGCTACAACGA CACTTGGA | 19645 |
| 3347 | GGCUAAUG G CAUGUAUU | 8948 | ACTCCATG GGCTAGCTACAACGA CCTTAGCC | 19646 |
| 3349 | CUAAGGGC A UUGAGUUC | 6726 | GAACTCCA GGCTAGCTACAACGA GCCCTTAG | 19647 |
| 3354 | GUCAUGGA G UUCUUGGC | 8949 | GCCAAGAA GGCTAGCTACAACGA TCCATGCC | 19648 |
| 3361 | AGUUCUUG G CAUCGCUA | 8950 | TCGCGATG GGCTAGCTACAACGA CAAGAACT | 19649 |
| 3363 | UUCUUUUC A UCGCUAUA | 6728 | TTTCGCGA GGCTAGCTACAACGA GCCAAGAA | 19650 |
| 3366 | UUGGCAUC G CGAAAGUG | 8951 | CACTTTCG GGCTAGCTACAACGA GATGCCAA | 19651 |
| 3372 | UCGCUAAA G UGUAUCCA | 8952 | TGGATACA GGCTAGCTACAACGA TTTCGCGA | 19652 |
| 3374 | UCGAAAGU G UAUCCACA | 8953 | TGTGGATA GGCTAGCTACAACGA ACTTTCGC | 19653 |
| 3376 | GAAAUUGU A UCCACAUG | 1976 | CCTGTGGA GGCTAGCTACAACGA ACACTTTC | 19654 |
| 3380 | GUUUAUCC A CAGUGACC | 6730 | GGTCCCTG GGCTAGCTACAACGA GGATACAC | 19655 |
| 3386 | CCACAUUU A CCUGUCUU | 8954 | CCGCCAGG GGCTAGCTACAACGA CCCTGTGG | 19656 |
| 3391 | UGGACCUG G CGGCACGA | 8955 | TCGTGCCG GGCTAGCTACAACGA CAGUTCCC | 19657 |
| 3394 | ACCUUGCG G CACUAAAU | 8956 | ATTTCGTG GGCTAGCTACAACGA CGCCAGGT | 19658 |
| 3396 | CUGUOGUC A CGAAAUAU | 6734 | ATATTTCG GGCTAGCTACAACGA GCCGCCAG | 19659 |
| 3401 | GGCACGAA A UAUCCUCU | 8957 | AGAGGATA GGCTAGCTACAACGA TTCGTGCC | 19660 |
| 3403 | CACUAAAU A UCCUCUGA | 1978 | TAAGAGGA GGCTAGCTACAACGA ATTTCGTG | 19661 |
| 3411 | AUCCUCUG A UCGGAGAA | 1982 | TTCTCCGA GGCTAGCTACAACGA AAGAGGAT | 19662 |
| 3422 | GGAGAGGA A CGUGGUUA | 8958 | TAACCACG GGCTAGCTACAACGA TCTTCTCC | 19663 |
| 3424 | AUAAUAAC G UGUUUAAA | 8959 | TTTAACCA GGCTAGCTACAACGA GTTCTTCT | 19664 |
| 3427 | AUAACUUU G UUAAAAUC | 8960 | GATTTTAA GGCTAGCTACAACGA CACGTTCT | 19665 |
| 3433 | UUGUUAAA A UCUGUGAC | 8961 | GTCACAGA GGCTAGCTACAACGA TTTAACCA | 19666 |
| 3437 | UAAAAUCU G UGACUUUU | 8962 | CAAAGTCA GGCTAGCTACAACGA AGATTTTA | 19667 |
| 3440 | AAUCUUUG A CUUUGGCU | 8963 | AGCCAAAG GGCTAGCTACAACGA CACAGATT | 19668 |
| 3446 | UGACUUUU G CUUUUCCC | 8964 | GGGCCAAG GGCTAGCTACAACGA CAAAGTCA | 19669 |
| 3451 | UUUUCUUU G CCCGGUAU | 8965 | ATCCCGGG GGCTAGCTACAACGA CAAGCCAT | 19670 |
| 3458 | UGCCCUUU A UAUUUAUA | 8966 | TATAAATA GGCTAGCTACAACGA CCCGGGCC | 19671 |
| 3460 | CCCUGGAU A UUAAUAAA | 1990 | TTTATAAA GGCTAGCTACAACGA ATCCCGGG | 19672 |
| 3464 | GGAUAUUU A UAAAGAUC | 1993 | GATCTTTA GGCTAGCTACAACGA AAATATCC | 19673 |
| 3470 | UUAUAAAG A UCCAGAUU | 8967 | AATCTGCA GGCTAGCTACAACGA CTTTATAA | 19674 |
| 3476 | ACAUCCAG A UHAUGUCA | 8968 | TGACATAA GGCTAGCTACAACGA CTGGATCT | 19675 |
| 3479 | UCCAGAUG A UGUCAGAA | 1997 | TTCTGACA GGCTAGCTACAACGA AATCTGGA | 19676 |
| 3481 | CAGAUUAU G UCAGAAAA | 8969 | TTTTCTGA GGCTAGCTACAACGA ATAATCTG | 19677 |
| 3494 | AAAAGGAG A UGCUCGCC | 8970 | GGCGAGCA GGCTAGCTACAACGA CTCCTTTT | 19678 |
| 3496 | AAGGAGAU G CUCGCCUC | 8971 | GAGGCGAG GGCTAGCTACAACGA ATCTCCTT | 19679 |
| 3500 | AGAUCCUC C CCUCCCUU | 8972 | AAGGGAGG GGCTAGCTACAACGA GAGCATCT | 19680 |
| 3513 | CCUUUGAA A UGGAUGGC | 8973 | GCCATCCA GGCTAGCTACAACGA TTCAAAGG | 19681 |
| 3517 | UGAAAUGG A UGGCCCCA | 8974 | TGGGGCCA GGCTAGCTACAACGA CCATTTCA | 19682 |
| 3520 | AAUGGAUG G CCCCAGAA | 8975 | TTCTGGGG GGCTAGCTACAACGA CATCCATT | 19683 |
| 3529 | CCCCAGAA A CAAUUUUU | 8976 | AAAAATTG GGCTAGCTACAACGA TTCTGGGG | 19684 |
| 3532 | CAGAAACA A UUUUUGAC | 8977 | GTCAAAAA GGCTAGCTACAACGA TGTTTCTG | 19685 |
| 3539 | AAUUUUUG A CAGAGUGU | 8978 | ACACTCTG GGCTAGCTACAACGA CAAAAATT | 19686 |
| 3544 | UUGACAGA G UGUACACA | 8979 | TGTGTACA GGCTAGCTACAACGA TCTGTCAA | 19687 |
| 3546 | GACAGAGU G UACACAAU | 8980 | ATTGTGTA GGCTAGCTACAACGA ACTCTGTC | 19688 |
| 3548 | CAGAGUGU A CACAAUCC | 2007 | GGATTGTG GGCTAGCTACAACGA ACACTCTG | 19689 |
| 3550 | GAGUGUAC A CAAUCCAG | 6758 | CTGGATTG GGCTAGCTACAACGA GTACACTC | 19690 |
| 3553 | UGUACACA A UCCAGAGU | 8981 | ACTCTGGA GGCTAGCTACAACGA TGTGTACA | 19691 |
| 3560 | AAUCCAGA G UGACGUCU | 8982 | AGACGTCA GGCTAGCTACAACGA TCTGGATT | 19692 |
| 3563 | CCAGAGUG A CGUCUGGU | 8983 | ACCAGACG GGCTAGCTACAACGA CACTCTGG | 19693 |
| 3565 | AGAGUGAC G UCUGGUCU | 8984 | AGACCAGA GGCTAGCTACAACGA GTCACTCT | 19694 |
| 3570 | GACGUCUG U UCUUUUGG | 8985 | CCAAAAGA GGCTAGCTACAACGA CAGACGTC | 19695 |
| 3578 | GUCUUUUG G UGUUUUGC | 8986 | GCAAAACA GGCTAGCTACAACGA CAAAAGAC | 19696 |
| 3580 | CUUUUGGU C UUUUGCUG | 8987 | CAGCAAAA GGCTAGCTACAACGA ACCAAAAG | 19697 |
| 3585 | GGUGUUUU G CUGUGGGA | 8988 | TCCCACAG GGCTAGCTACAACGA AAAACACC | 19698 |
| 3588 | GUUUUGCU G UGGGAAAU | 8989 | ATTTCCCA GGCTAGCTACAACGA AGCAAAAC | 19699 |
| 3595 | UGUGGGAA A UAUUUUCC | 8990 | GGAAAATA GGCTAGCTACAACGA TTCCCACA | 19700 |
| 3597 | UGGGAAAU A UUUUCCUU | 2017 | AAGGAAAA GGCTAGCTACAACGA ATTTCCCA | 19701 |
| 3608 | UUCCUUAG G UGCUUCUC | 8991 | GAGAAGCA GGCTAGCTACAACGA CTAAGGAA | 19702 |
| 3610 | CCUUAGGU G CUUCUCCA | 8992 | TGGAGAAG GGCTAGCTACAACGA ACCTAAGG | 19703 |
| 3618 | GCUUCUCC A UAUCCUGG | 6770 | CCAGGATA GGCTAGCTACAACGA GGAGAAGC | 19704 |
| 3620 | UUCUCCAU A UCCUGGUG | 2027 | CCCCAGGA GGCTAGCTACAACGA ATGGAGAA | 19705 |
| 3628 | AUCCUGUG G UAAAGAUU | 8993 | AATCTTTA GGCTAGCTACAACGA CCCAGGAT | 19706 |
| 3634 | GGGUAAAG A UUGAUGAA | 8994 | TTCATCAA GGCTAGCTACAACGA CTTTACCC | 19707 |
| 3638 | AAAGAUUG A UGAAGAAU | 8995 | ATTCTTCA GGCTAGCTACAACGA CAATCTTT | 19708 |
| 3645 | GAUGAAGA A UUUUGUAG | 8996 | CTACAAAA GGCTAGCTACAACGA TCTTCATC | 19709 |
| 3650 | AGAAUUUU C UAGGCGAU | 8997 | ATCGCCTA GGCTAGCTACAACGA AAAATTCT | 19710 |
| 3654 | UUUUGUAG C CGAUUGAA | 8998 | TTCAATCG GGCTAGCTACAACGA CTACAAAA | 19711 |
| 3657 | UGUAGGCG A UUGAAAGA | 8999 | TCTTTCAA GGCTAGCTACAACGA CGCCTACA | 19712 |
| 3670 | AAGAAGGA A CUAGAAUG | 9000 | CATTCTAG GGCTAGCTACAACGA TCCTTCTT | 19713 |
| 3676 | GAACUAGA A UGAGGGCC | 9001 | GGCCCTCA GGCTAGCTACAACGA TCTAGTTC | 19714 |
| 3682 | GAAUGAGG C CCCCUGAU | 9002 | ATCAGGGG GGCTAGCTACAACGA CCTCATTC | 19715 |
| 3689 | GUCOCCUG A UUAUACUA | 9003 | TAGTATAA GGCTAGCTACAACGA CAGGUCCG | 19716 |
| 3692 | CCCUGAUU A UACUACAC | 2038 | GTGTAGTA GGCTAGCTACAICGA AATCACGG | 19717 |
| 3694 | CUGAUUAU A CUACACCA | 2039 | TOCTUTAG GGCTAGCTACAACGA ATAATCAG | 19718 |
| 3697 | AUUAUACU A CACCAGAA | 2040 | TTCTGGTC GGCTAGCTACAACGA AGTATAAT | 19719 |
| 3699 | UAUACUAC A CCAGAAAU | 6779 | ATTTCTGG GGCTAGCTACAACGA GTACTATA | 19720 |
| 3706 | CACCAGAA A UGUACCAG | 9004 | CTGGTACA GGCTAGCTACAACGA TTCTGGTC | 19721 |
| 3708 | CCAGAAAU C UACCAGAC | 9005 | GTCTCGTA GGCTAGCTACAACGA ATTTCTGG | 19722 |
| 3710 | AGAAAUCU A CCAGACCA | 2041 | TGGTCTGG GGCTAGCTACAACGA ACATTTCT | 19723 |
| 3715 | UGUACCAG A CCAUGCUG | 9006 | CAGCATGG GGCTAGCTACAACGA CTGGTACA | 19724 |
| 3718 | ACCAGACC A UGCUGGAC | 6785 | GTCCAGCA GGCTAGCTACAACGA GGTCTGGT | 19725 |
| 3720 | CAGACCAU G CUGGACUG | 9007 | CAGTCCAG GGCTAGCTACAACGA ATGGTCTG | 19726 |
| 3725 | CAUGCUGG A CUGCUGGC | 7740 | GCCAGCAG GGCTAGCTACAACGA CCAGCATG | 17305 |
| 3728 | GCUGGACU G CUGGCACG | 9008 | CGTGCCAG GGCTAGCTACAACGA AGTCCAGC | 19727 |
| 3732 | GACUOCUG G CACGGGGA | 9009 | TCCCCGTG GGCTAGCTACAACGA CAGCAGTC | 19728 |
| 3734 | CUGCUGGC A CGGGGAGC | 6788 | GCTCCCCG GGCTAGCTACAACGA UCCACCAG | 19729 |
| 3741 | CACCUGGA G CCCAGUCA | 9010 | TGACTGGG GGCTAGCTACAACGA TCCCCGTG | 19730 |
| 3746 | GGAGCCCA G UCAGACAC | 9011 | GTCTCTGA GGCTAGCTACAACGA TGGGCTCC | 19731 |
| 3753 | AGUCAGAG A CCCACGUU | 9012 | AACCTGGG GGCTAGCTACAACGA CTCTGACT | 19732 |
| 3757 | AGAGACCC A CGUUUUCA | 6795 | TGAAAACG GGCTAGCTACAACGA GGGTCTCT | 19733 |
| 3759 | AGACCCAC C UUUUCAGA | 9013 | TCTGAAAA GGCTACCTACAACGA GTGGGTCT | 19734 |
| 3768 | UUUUCAGA G UUGGUGGA | 9014 | TCCACCAA GGCTAGCTACAACGA TCTGAAAA | 19735 |
| 3772 | CAGAGUUG G UGGAACAU | 9015 | ATGTTCCA GGCTAGCTACAACGA CAACTCTG | 19736 |
| 3777 | GUGGUGGA A CAUUUGGG | 9016 | CCCAAATG GGCTAGCTACAACGA TCCACCAA | 19737 |
| 3779 | GGUGGAAC A UUUGGGAA | 6797 | TTCCCAAA GGCTAGCTACAACGA GTTCCACC | 19738 |
| 3788 | UUUGGGAA A UCUCUUGC | 9017 | GCAAGAGA GGCTAGCTACAACGA TTCCCAAA | 19739 |
| 3795 | AAUCUCUU G CAAGCUAA | 9018 | TTAGCTTG GGCTAGCTACAACGA AAGAGATT | 19740 |
| 3799 | UCUUGCAA G CUAAUGCU | 9019 | ACCATTAG GGCTAGCTACAACGA TTGCAAGA | 19741 |
| 3803 | GCAAGCUA A UGCUCAGC | 9020 | GCTGAGCA GGCTAGCTACAACGA TAGCTTGC | 19742 |
| 3805 | AAGCUAAU G CUCACCAG | 9021 | CTGCTGAG GGCTAGCTACAACGA ATTAGCTT | 19743 |
| 3810 | AAUGCUCA C CAGGAUGG | 9022 | CCATCCTG GGCTAGCTACAACGA TGAGCATT | 19744 |
| 3815 | UCAGCAGG A UGGCAAAG | 9023 | CTTTGCCA GGCTAGCTACAACGA CCTGCTGA | 19745 |
| 3818 | OCAGGAUG G CAAAGACU | 9024 | AGTCTTTG GGCTAGCTACAACGA CATCCTGC | 19746 |
| 3824 | UGGCAAAG A CUACAUUG | 9025 | CAATGTAG GGCTAGCTACAACGA CTTTGCCA | 19747 |
| 3827 | CAAAGACU A CAUUGUUC | 2055 | GAACAATG GGCTAGCTACAACGA AGTCTTTG | 19748 |
| 3829 | AAGACUAC A UUGUUCUU | 6807 | AAGAACAA GGCTAGCTACAACGA GTAGTCTT | 19749 |
| 3832 | ACUACAUU G UUCUUCCG | 9026 | CGGAAGAA GGCTAGCTACAACGA AATGTAGT | 19750 |
| 3841 | UUCUUCCG A UAUCAGAG | 9027 | CTCTGATA GGCTAGCTACAACGA CGGAAGAA | 19751 |
| 3843 | CUUCCGAU A UCAGAGAC | 2061 | GTCTCTGA GGCTAGCTACAACGA ATCGGAAG | 19752 |
| 3850 | UAUCAGAG A CUUUGAGC | 9028 | GCTCAAAG GGCTAGCTACAACGA CTCTGATA | 19753 |
| 3857 | GACUUUGA G CAUGGAAG | 9029 | CTTCCATG GGCTAGCTACAACGA TCAAAGTC | 19754 |
| 3859 | CUUUGAGC A UGGAAGAG | 6812 | CTCTTCCA GGCTAGCTACAACGA GCTCAAAG | 19755 |
| 3869 | GGAAGAGG A UUCUGGAC | 9030 | GTCCAGAA GGCTAGCTACAACGA CCTCTTCC | 19756 |
| 3876 | GAUUCUGG A CUCUCUCU | 9031 | AGAGAGAG GGCTAGCTACAACGA CCAGAATC | 19757 |
| 3885 | CUCUCUCU C CCUACCUC | 9032 | GAGGTAGG GGCTAGCTACAACGA AGAGAGAG | 19758 |
| 3889 | CUCUGCCU A CCUCACCU | 2070 | AGGTGAGG GGCTAGCTACAACGA AGGCAGAG | 19759 |
| 3894 | CCUACCUC A CCUGUUUC | 6822 | GAAACAGG GGCTAGCTACAACGA GAGGTAGG | 19760 |
| 3898 | CCUCACCU C UUUCCUGU | 9033 | ACAGGAAA GGCTAGCTACAACGA AGGTGAGG | 19761 |
| 3905 | UGUUUCCU C UAUGGAGG | 9034 | CCTCCATA GGCTAGCTACAACGA AGGAAACA | 19762 |
| 3907 | UUUCCUGU A UGGAGGAG | 2075 | CTCCTCCA GGCTAGCTACAACGA ACAGGAAA | 19763 |
| 3922 | AGGAGGAA C UAUGUGAC | 9035 | GTCACATA GGCTAGCTACAACCA TTCCTCCT | 19764 |
| 3924 | GAGGAAGU A UGUGACCC | 2076 | GCCTCACA GGCTAGCTACAACGA ACTTCCTC | 19765 |
| 3926 | GCAAGUAU C UGACCCCA | 9036 | TGGGCTCA GGCTAGCTACAACGA ATACTTCC | 19766 |
| 3929 | AGUAUGUG A CCCCAAAU | 9037 | ATTTGGGG GGCTAGCTACAACGA CACATACT | 19767 |
| 3936 | CACCCCAA A UUCCAUUA | 9038 | TAATCGAA GGCTAGCTACAACGA TTCCGGTC | 19768 |
| 3941 | CAAAUUCC A UUAUGACA | 6832 | TCTCATAA GGCTAGCTACAACGA GCAATTTG | 19769 |
| 3944 | AUUCCAUU A UGACAACA | 2080 | TGTTCTCA GGCTACCTACAACGA AATGGAAT | 19770 |
| 3947 | CCAUUAUG A CAACACAG | 9039 | CTGTGTTG GGCTAGCTACAACGA CATAATGG | 19771 |
| 3950 | UUAUGACA A CACACCAG | 9040 | CTGCTGTG GGCTAGCTACAACGA TGTCATAA | 19772 |
| 3952 | AUGACAAC A CAGCAGGA | 6834 | TCCTGCTG GGCTAGCTACAACGA GTTGTCAT | 19773 |
| 3955 | ACAACACA C CAGGAAUC | 9041 | GATTCCTG GGCTAGCTACAACGA TGTGTTGT | 19774 |
| 3961 | CAGCAGGA A UCAGUCAG | 9042 | CTGACTGA GGCTAGCTACAACGA TCCTGCTG | 19775 |
| 3965 | AGGAAUCA G UCAGUAUC | 9043 | GATACTGA GGCTAGCTACAACGA TGATTCCT | 19776 |
| 3969 | AUCAGUCA G UAUCUGCA | 9044 | TGCAGATA GGCTAGCTACAACGA TGACTGAT | 19777 |
| 3971 | CAGUCAGU A UCUGCAGA | 2083 | TCTGCAGA GGCTAGCTACAACGA ACTGACTG | 19778 |
| 3975 | CAGUAUCU G CAGAACAG | 9045 | CTGTTCTG GGCTAGCTACAACGA AGATACTG | 19779 |
| 3980 | UCUGCAGA A CAGUAAGC | 9046 | GCTTACTG GGCTAGCTACAACGA TCTGCAGA | 19780 |
| 3983 | GCAGAACA G UAAGCGAA | 9047 | TTCGCTTA GGCTAGCTACAACGA TGTTCTGC | 19781 |
| 3987 | AACAGUAA C CGAAAGAG | 9048 | CTCTTTCG GGCTAGCTACAACGA TTACTGTT | 19782 |
| 3995 | GCGAAAGA G CCGGCCUG | 9049 | CAGGCCGG GGCTAGCTACAACGA TCTTTCGC | 19783 |
| 3999 | AAGAGCCG G CCUGUGAG | 9050 | CTCACAGG GGCTAGCTACAACGA CGCCTCTT | 19784 |
| 4003 | GCCGGCCU G UGAGUGUA | 9051 | TACACTCA GGCTAGCTACAACGA AGGCCGGC | 19785 |
| 4007 | GCCUGUGA C UGUAAAAA | 9052 | TTTTTACA GGCTAGCTACAACGA TCACAGGC | 19786 |
| 4009 | CUGUGACU G UAAAAACA | 9053 | TGTTTTTA GCCTACCTACAACGA ACTCACAC | 19787 |
| 4015 | GUGUAAAA A CAUUUGAA | 9054 | TTCAAATG GGCTAGCTACAACGA TTTTACAC | 19788 |
| 4017 | CUAAAAAC A UUUGAAGA | 6845 | TCTTCAAA GGCTAGCTACAACGA GTTTTTAC | 19789 |
| 4025 | AUUUGAAG A UAUCCCGU | 9055 | ACGGGATA GGCTAGCTACAACGA CTTCAAAT | 19790 |
| 4027 | UUCAACAU A UCCCCUUA | 2089 | TAACGCCA GGCTACCTACAACGA ATCTTCAA | 19791 |
| 4032 | GAUAUCCC G UUAGAAGA | 9056 | TCTTCTAA GGCTAGCTACAACGA GGCATATC | 19792 |
| 4041 | UUAGAAGA A CCAGAAGU | 9057 | ACTTCTGG GGCTAGCTACAACGA TCTTCTAA | 19793 |
| 4048 | AACCAGAA C UAAAACUA | 9058 | TACTTTTA GGCTAGCTACAACGA TTCTGGTT | 19794 |
| 4054 | AAGUAAAA G UAAUCCCA | 9059 | TOGGATTA GGCTAGCTACAACGA TTTTACTT | 19795 |
| 4057 | UAAAAGUA A UCCCACAU | 9060 | ATCTGGGA GGCTAGCTACAACGA TACTTTTA | 19796 |
| 4064 | AAUCCCAC A UGACAACC | 9061 | GCTTCTCA GGCTAGCTACAACGA CTGCGATT | 19797 |
| 4067 | CCCAGAUG A CAACCACA | 9062 | TCTGGTTG GGCTAGCTACAACGA CATCTGGG | 19798 |
| 4070 | AGAUGACA A CCAGACGG | 9063 | CCGTCTGG GGCTACCTACAACGA TGTCATCT | 19799 |
| 4075 | ACAACCAG A CGGACAGU | 9064 | ACTGTCCG GGCTAGCTACAACGA CTGGTTGT | 19800 |
| 4079 | CCAGACGG A CAGUGGUA | 9065 | TACCACTG GGCTAGCTACAACGA CCGTCTGG | 19801 |
| 4082 | CACGGACA G UGGUAUGG | 9066 | CCATACCA GGCTAGCTACAACGA TGTCCGTC | 19802 |
| 4085 | GGACAGUG C UAUGGUUC | 9067 | GAACCATA GGCTAGCTACAACGA CACTGTCC | 19803 |
| 4087 | ACACUGGU A UGCUUCUU | 2096 | AAGAACCA GGCTAGCTACAACGA ACCACTGT | 19804 |
| 4090 | GUGCUAUG G UUCUUGCC | 9068 | GGCAAGAA GGCTAGCTACAACGA CATACCAC | 19805 |
| 4096 | UGGUUCUU C CCUCAGAA | 9069 | TTCTGAGG GGCTAGCTACAACGA AAGAACCA | 19806 |
| 4107 | UCAGAAGA G CUGAAAAC | 9070 | GTTTTCAG GGCTAGCTACAACGA TCTTCTGA | 19807 |
| 4114 | AGCUCAAA A CUUUCGAA | 9071 | TTCCAAAG GGCTAGCTACAACGA TTTCAGCT | 19808 |
| 4124 | UUUGGAAG A CACAACCA | 9072 | TGGTTCTC GGCTACCTACAACGA CTTCCAAA | 19809 |
| 4129 | AAGACAGA A CCAAAUUA | 9073 | TAATTTGG GGCTAGCTACAACGA TCTCTCTT | 19810 |
| 4134 | AGAACCAA A UUAUCUCC | 9074 | GGACATAA GGCTAGCTACAACGA TTCGTTCT | 19811 |
| 4137 | ACCAAAUU A UCUCCAUC | 2104 | GATGCAGA GGCTAGCTACAACGA AATTTGGT | 19812 |
| 4143 | UUAUCUCC A UCUUUUGG | 6868 | CCAAAAGA GGCTAGCTACAACGA GGACATAA | 19813 |
| 4151 | AUCUUUUG C UCGAAUGC | 9075 | CCATTCCA GGCTAGCTACAACGA CAAAAGAT | 19814 |
| 4156 | UUGCUGGA A UGCUCCCC | 9076 | GGGCACCA GGCTAGCTACAACGA TCCACCAA | 19815 |
| 4159 | GUCGAAUG C UCCCCAGC | 9077 | CCTCGGCA GGCTAGCTACAACGA CATTCCAC | 19816 |
| 4161 | GGAAUGCU C CCCAGCAA | 9078 | TTGCTGCG GGCTAGCTACAACGA ACCATTCC | 19817 |
| 4166 | CGUGCCCA C CAAAACCA | 9079 | TCCTTTTC GGCTAGCTACAACGA TCCCCACC | 19818 |
| 4172 | CACCAAAA C CACCCACU | 9080 | ACTCCCTC GGCTAGCTACAACCA TTTTCCTC | 19819 |
| 4179 | AGOACOGA G UCUGUGGC | 9081 | GCCACAGA GGCTAGCTACAACGA TCCCTGCT | 19820 |
| 4183 | GGGAGUCU G UGGCAUCU | 9082 | AGATOCCA GGCTAGCTACAACGA AGACTCCC | 19821 |
| 4186 | AGUCUGUG C CAUCUGAA | 9083 | TTCAGATG GGCTAGCTACAACGA CACAGACT | 19822 |
| 4188 | UCUGUGGC A UCUGAAGG | 6876 | CCTTCAGA GGCTAGCTACAACGA GCCACAGA | 19823 |
| 4196 | AUCUGAAG G CUCAAACC | 9084 | GGTTTGAG GGCTAGCTACAACGA CTTCAGAT | 19824 |
| 4202 | AGGCUCAA A CCAGACAA | 9085 | TTGTCTGG GGCTAGCTACAACGA TTGAGCCT | 19825 |
| 4207 | CAAACCAG A CAAGCGGC | 9086 | GCCGCTTG GGCTAGCTACAACGA CTGGTTTG | 19826 |
| 4211 | CCAGACAA G CGGCUACC | 9087 | GGTAGCCG GGCTAGCTACAACGA TTGTCTGG | 19827 |
| 4214 | GACAAGCG G CUACCAGU | 9088 | ACTGGTAG GGCTAGCTACAACGA CGCTTGTC | 19828 |
| 4217 | AAGCGGCU A CCAGUCCG | 2114 | CGGACTGG GGCTAGCTACAACGA AGCCGCTT | 19829 |
| 4221 | GGCUACCA G UCCGGAUA | 9089 | TATCCGGA GGCTAGCTACAACGA TGGTAGCC | 19830 |
| 4227 | CAGUCCUG A UAUCACUC | 9090 | GAGTGATA GGCTAGCTACAACGA CCGGACTG | 19831 |
| 4229 | GUCCGGAU A UCACUCCG | 2116 | CGGAGTGA GGCTAGCTACAACGA ATCCGGAC | 19832 |
| 4232 | CGGAUAUC A CUCCGAUG | 6887 | CATCGGAG GGCTAGCTACAACGA GATATCCG | 19833 |
| 4238 | UCACUCCG A UGACACAG | 9091 | CTGTGTCA GGCTAGCTACAACGA CGGAGTGA | 19834 |
| 4241 | CUCCGAUG A CACAGACA | 9092 | TGTCTGTG GGCTAGCTACAACGA CATCGGAG | 19835 |
| 4243 | CCGAUGAC A CAGACACC | 6890 | GGTGTCTG GGCTAGCTACAACGA GTCATCGG | 19836 |
| 4247 | UGACACAG A CACCACCG | 9093 | CGGTGGTG GGCTAGCTACAACGA CTGTGTCA | 19837 |
| 4249 | ACACAGAC A CCACCGUG | 6892 | CACGGTGG GGCTAGCTACAACGA GTCTGTGT | 19838 |
| 4252 | CAGACACC A CCGUGUAC | 6894 | OTACACOG GGCTAGCTACAACGA GGTGTCTG | 19839 |
| 4255 | ACACCACC G UGUACUCC | 9094 | GGAGTACA GGCTAGCTACAACGA GGTGGTGT | 19840 |
| 4257 | ACCACCGU C UACUCCAG | 9095 | CTGGAGTA GGCTAGCTACAACGA ACGGTGGT | 19841 |
| 4259 | CACCGUGU A CUCCAGUG | 2119 | CACTGGAG GGCTAGCTACAACGA ACACGGTG | 19842 |
| 4265 | GUACUCCA C UGAGGAAG | 9096 | CTTCCTCA GGCTAGCTACAACGA TGGAGTAC | 19843 |
| 4273 | GUGAGGAA C CAGAACUU | 9097 | AAGTTCTG GGCTAGCTACAACGA TTCCTCAC | 19844 |
| 4278 | GAAGCAGA A CUUUUAAA | 9098 | TTTAAAAG GGCTAGCTACAACGA TCTGCTTC | 19845 |
| 4287 | CUUUUAAA C CUGAUAGA | 9099 | TCTATCAG GGCTAGCTACAACGA TTTAAAAG | 19846 |
| 4291 | UAAAGCUG A UAGAGAUU | 9100 | AATCTCTA GGCTAGCTACAACGA CAGCTTTA | 19847 |
| 4297 | UGAUAGAG A UUGGAGUG | 9101 | CACTCCAA GGCTAGCTACAACGA CTCTATCA | 19848 |
| 4303 | AGAGUGGA G UGCAAACC | 9102 | GGTTTGCA GGCTAGCTACAACGA TCCAATCT | 19849 |
| 4305 | AUUGGAGU G CAAACCGG | 9103 | CCGGTTTG GGCTAGCTACAACGA ACTCCAAT | 19850 |
| 4309 | GAGUGCAA A CCGGUAGC | 9104 | GCTACCGG GGCTAGCTACAACGA TTGCACTC | 19851 |
| 4313 | GCAAACCG C UAGCACAG | 9105 | CTGTGCTA GGCTAGCTACAACGA CGGTTTGC | 19852 |
| 4316 | AACCGGUA G CACAGCCC | 9106 | GGGCTGTG GGCTAGCTACAACGA TACCGGTT | 19853 |
| 4318 | CCGGUAGC A CAGCCCAG | 6904 | CTGGGCTG GGCTAGCTACAACGA GCTACCGG | 19854 |
| 4321 | GUAGCACA G CCCAGAUU | 9107 | AATCTGGG GGCTAGCTACAACGA TGTGCTAC | 19855 |
| 4327 | CAGCCCAG A UUCUCCAG | 9108 | CTGGAGAA GGCTAGCTACAACGA CTGGGCTG | 19856 |
| 4335 | AUUCUCCA C CCUGACUC | 9109 | GAGTCAGG GGCTAGCTACAACGA TGGAGAAT | 19857 |
| 4340 | CCAGCCUG A CUCGGGGA | 9110 | TCCCCGAG GGCTAGCTACAACGA CAGGCTGG | 19858 |
| 4348 | ACUCGGGG A CCACACUG | 9111 | CAGTGTGG GGCTAGCTACAACGA CCCCGAGT | 19859 |
| 4351 | CGGGGACC A CACUGAGC | 6916 | GCTCAGTG GGCTAGCTACAACGA GGTCCCCG | 19860 |
| 4353 | GGGACCAC A CUGAGCUC | 6917 | GAGCTCAG GGCTAGCTACAACGA GTGGTCCC | 19861 |
| 4358 | CACACUGA C CUCUCCUC | 9112 | GAGGAGAG GGCTAGCTACAACGA TCAGTGTG | 19862 |
| 4369 | CUCCUCCU C UUUAAAAG | 9113 | CTTTTAAA GGCTAGCTACAACGA AGGAGGAG | 19863 |
| 4381 | AAAAGGAA C CAUCCACA | 9114 | TGTGGATG GGCTAGCTACAACGA TTCCTTTT | 19864 |
| 4383 | AAGGAAGC A UCCACACC | 6925 | GGTGTGGA GGCTAGCTACAACGA GCTTCCTT | 19865 |
| 4387 | AAGCAUCC A CACCCCAA | 6927 | TTGGGGTG GGCTAGCTACAACGA GGATGCTT | 19866 |
| 4389 | GCAUCCAC A CCCCAACU | 6928 | AGTTGGGG GGCTAGCTACAACGA GTGGATGC | 19867 |
| 4395 | ACACCCCA A CUCCCGGA | 9115 | TCCGGGAG GGCTAGCTACAACGA TGGGGTGT | 19868 |
| 4403 | ACUCCCGG A CAUCACAU | 9116 | ATGTGATG GGCTAGCTACAACGA CCGGGAGT | 19869 |
| 4405 | UCCCGGAC A UCACAUGA | 6936 | TCATGTGA GGCTAGCTACAACGA GTCCGGCA | 19870 |
| 4408 | CGGACAUC A CAUGAGAG | 6937 | CTCTCATG GGCTAGCTACAACGA GATGTCCG | 19871 |
| 4410 | GACAUCAC A UGAGAGGU | 6938 | ACCTCTCA GGCTAGCTACAACGA GTGATGTC | 19872 |
| 4417 | CAUGAGAG G UCUGCUCA | 9117 | TGAGCAGA GGCTAGCTACAACGA CTCTCATG | 19873 |
| 4421 | AGAGGUCU C CUCAGAUU | 9118 | AATCTGAG GGCTAGCTACAACGA ACACCTCT | 19874 |
| 4427 | CUGCUCAG A UUUUGAAG | 9119 | CTTCAAAA GGCTAGCTACAACGA CTGAGCAG | 19875 |
| 4435 | AUUUUGAA G UGUUGUUC | 9120 | GAACAACA GGCTAGCTACAACGA TTCAAAAT | 19876 |
| 4437 | UUUGAAGU C UUGUUCUU | 9121 | AAGAACAA GGCTAGCTACAACGA ACTTCAAA | 19877 |
| 4440 | GAAGUCUU G UUCUUUCC | 9122 | GGAAACAA GGCTAGCTACAACGA AACACTTC | 19878 |
| 4449 | UUCUUUCC A CCAGCAGG | 6944 | CCTGCTGG GGCTAGCTACAACGA GCAAAGAA | 19879 |
| 4453 | UUCCACCA C CAGCAACU | 9123 | ACTTCCTG GGCTAGCTACAACGA TCGTGGAA | 19880 |
| 4460 | ACCACGAA C UAGCCGCA | 9124 | TGCGGCTA GGCTAGCTACAACGA TTCCTGCT | 19881 |
| 4463 | AGCAAGUA G CCGCAUUU | 9125 | AAATGCGG GGCTAGCTACAACGA TACTTCCT | 19882 |
| 4466 | AAGUACCC C CAUUUGAU | 9126 | ATCAAATG GGCTAGCTACAACGA CGCTACTT | 19883 |
| 4468 | CUAGCCGC A UUUGAUUU | 6949 | AAATCAAA GGCTAGCTACAACGA GCGCCTAC | 19884 |
| 4473 | CGCAUUUC A UUUUCAUU | 9127 | AATCAAAA GGCTAGCTACAACGA CAAATGCG | 19885 |
| 4479 | UCAUUUUC A UUUCGACA | 6950 | TGTCCAAA GGCTAGCTACAACGA CAAAATCA | 19886 |
| 4485 | UCAUUUCG A CAACAGAA | 9128 | TTCTCTTG GGCTAGCTACAACGA CGAAATCA | 19887 |
| 4488 | UUUCGACA A CAGAAAAA | 9129 | TTTTTCTG GGCTAGCTACAACGA TGTCCAAA | 19888 |
| 4499 | CAAAAAGG A CCUCCGAC | 9130 | CTCCCACG GGCTAGCTACAACGA CCTTTTTC | 19889 |
| 4506 | GACCUCCG A CUGCAGGG | 9131 | CCCTGCAC GGCTAGCTACAACGA CCGACGTC | 19890 |
| 4509 | CUCGCACU C CACGGAGC | 9132 | GCTCCCTG GGCTAGCTACAACGA AGTCCCAC | 19891 |
| 4516 | UCCAGGGA G CCAGUCUU | 9133 | AAGACTGG GGCTAGCTACAACGA TCCCTCCA | 19892 |
| 4520 | GCCACCCA G UCUCCUAG | 9134 | CTACAAGA GGCTAGCTACAACGA TCCCTCCC | 19893 |
| 4529 | UCUUCUAC G CAUAUCCU | 9135 | AGCATATC GGCTAGCTACAACGA CTAGAAGA | 19894 |
| 4531 | UUCUAGGC A UAUCCUGG | 9136 | CCAGCATA GGCTAGCTACAACGA GCCTAGAA | 19895 |
| 4533 | CUAGGCAU A UCCUGGAA | 9137 | TTCCAGGA GGCTAGCTACAACGA ATCCCTAC | 19896 |
| 4545 | UGGAACAG C CUUGUCAC | 9138 | GTCACAAC GGCTAGCTACAACGA CTCTTCCA | 19897 |
| 4549 | AGAGGCUU C UGACCCAA | 9139 | TTGCCTCA GGCTAGCTACAACGA AAGCCTCT | 19898 |
| 4552 | GCCUUGUG A CCCAACAA | 9140 | TTCTTGGG GGCTAGCTACAACGA CACAACCC | 19899 |
| 4560 | ACCCAAGA A UGUGUCUG | 9141 | CAGACACA GGCTAGCTACAACGA TCTTGGGT | 19900 |
| 4562 | CCAAGAAU G UGUCUGUG | 9142 | CACACACA GGCTAGCTACAACGA ATTCTTCG | 19901 |
| 4564 | AAGAAUGU C UCUCUGUC | 9143 | CACACAGA GGCTAGCTACAACGA ACATTCTT | 19902 |
| 4568 | AUGUCUCU C UCUCUUCU | 9144 | ACAACACA GGCTAGCTACAACGA AGACACAT | 19903 |
| 4570 | GUCUCUCU C UCUUCUCC | 9145 | CGAGAAGA GGCTAGCTACAACGA ACAGACAC | 19904 |
| 4581 | UUCUCCCA C UCUUCACC | 9146 | CCTCAACA GGCTAGCTACAACGA TGGGAGAA | 19905 |
| 4583 | CUCCCACU C UGGACCUC | 9147 | CACCTCAA GGCTAGCTACAACGA ACTGGCAC | 19906 |
| 4587 | CACUCUUC A CCUCAUCC | 9148 | GCATCACG GGCTAGCTACAACGA CAACACTC | 19907 |
| 4592 | GUCACCUG A UCCUCUUU | 9149 | AAAGACCA GGCTAGCTACAACGA CACCTCAA | 19908 |
| 4605 | CUUUUUUC A UUCAUUUA | 9150 | TAAATCAA GGCTAGCTACAACGA GAAAAAAG | 19909 |
| 4609 | UUUCAUUC A UUUAAAAA | 9151 | TTTTTAAA GGCTAGCTACAACGA CAATCAAA | 19910 |
| 4618 | UUUAAAAA G CAUUAUCA | 9152 | TCATAATC GGCTAGCTACAACGA TTTTTAAA | 19911 |
| 4620 | UAAAAACC A UUAUCAUC | 9153 | CATCATAA GGCTAGCTACAACGA CCTTTTTA | 19912 |
| 4623 | AAACCAUU A UCAUCCCC | 9154 | CCGCATGA GGCTAGCTACAACGA AATGCTTT | 19913 |
| 4626 | GCAUUAUC A UGCCCCUG | 9155 | CACGCCCA GGCTAGCTACAACGA GATAATCC | 19914 |
| 4628 | AUUAUCAU C CCCCUCCU | 9156 | ACCACCCC GGCTAGCTACAACGA ATCATAAT | 19915 |
| 4634 | AUCCCCCU C CUCCCCCU | 9157 | ACCCCCAC GGCTAGCTACAACGA ACCCCCAT | 19916 |
| 4637 | CCCCUGCU C CCGCUCUC | 9158 | GAGACCCG GGCTAGCTACAACGA ACCACCCG | 19917 |
| 4641 | UGCUGCGG G UCUCACCA | 9159 | TGGTCAGA GGCTACCTACAACGA CCGCAGCA | 19918 |
| 4646 | CGGGUCUC A CCAUGGGU | 9160 | ACCCATGG GCCTAGCTACAACGA GAGACCCG | 19919 |
| 4649 | GUCUCACC A UGGGUUUA | 9161 | TAAACCCA GGCTAGCTACAACGA GGTCAGAC | 19920 |
| 4653 | CACCAUGG G UUUAGAAC | 9162 | GTTCTAAA GGCTAGCTACAACGA CCATGGTG | 19921 |
| 4660 | GGUUUAGA A CAAAGAGC | 9163 | GCTCTTTG GGCTAGCTACAACGA TCTAAACC | 19922 |
| 4667 | AACAAAGA C CUUCAACC | 9164 | CCTTGAAG GGCTAGCTACAACGA TCTTTCTT | 19923 |
| 4674 | AGCUUCAA C CAAUCGCC | 9165 | CGCCATTC GGCTACCTACAACGA TTGAAGCT | 19924 |
| 4677 | UUCAAGCA A UGGCCCCA | 9166 | TGGGGCCA GGCTAGCTACAACGA TGCTTGAA | 19925 |
| 4680 | AAGCAAUG G CCCCAUCC | 9167 | GGATGGCG GGCTAGCTACAACGA CATTCCTT | 19926 |
| 4685 | AUGGCCCC A UCCUCAAA | 9168 | TTTGAGGA GGCTAGCTACAACGA CUGUCCAT | 19927 |
| 4697 | UCAAAGAA C UACCACUA | 9169 | TACTUCTA GGCTACCTACAACGA TTCTTTGA | 19928 |
| 4700 | AACAACUA G CAGUACCU | 9170 | ACCTACTC GGCTACCTACAACGA TACTTCTT | 19929 |
| 4703 | AAGUACCA C UACCUCCG | 9171 | CCCAGGTA GGCTACCTACAACGA TCCTACTT | 19930 |
| 4705 | CUACCACU A CCUCGCGA | 9172 | TCCCCACG GGCTAGCTACAACGA ACTGCTAC | 19931 |
| 4714 | CCUGGCGA C CUGACACU | 9173 | ACTCTCAC GGCTAGCTACAACGA TCCCCAGC | 19932 |
| 4718 | CCCACCUC A CACUUCUC | 9174 | CAGAAGTC GGCTACCTACAACGA CACCTCCC | 19933 |
| 4720 | GAGCUGAC A CUUCUCUA | 9175 | TACAGAAG GGCTAGCTACAACGA GTCAGCTC | 19934 |
| 4726 | ACACUUCU G UAAAACUA | 9176 | TAGTTTTA GGCTACCTACAACGA ACAAGTCT | 19935 |
| 4731 | UCUCUAAA A CUAGAAGA | 9177 | TCTTCTAG GGCTAGCTACAACGA TTTACAGA | 19936 |
| 4739 | ACUAGAAC A UAAACCAG | 9178 | CTGGTTTA GCCTAGCTACAACGA CTTCTAGT | 19937 |
| 4743 | CAAGAUAA A CCAGGCAA | 9179 | TTGCCTGG GGCTAGCTACAACGA TTATCTTC | 19938 |
| 4748 | UAAACCAG G CAACCUAA | 9180 | TTACGTTC GGCTAGCTACAACGA CTGCTTTA | 19939 |
| 4751 | ACCACGCA A CCUAACUG | 9181 | CACTTACC GGCTAGCTACAACGA TCCCTCCT | 19940 |
| 4753 | CAGCCAAC G UAACUGUU | 9182 | AACACTTA GGCTAGCTACAACGA GTTCCCTC | 19941 |
| 4757 | CAACCUAA G UCUUCGAC | 9183 | CTCGAACA GGCTAGCTACAACGA TTACGTTC | 19942 |
| 4759 | ACCUAAGU C UUCGACCU | 9184 | ACCTCGAA GGCTAGCTACAACGA ACTTACGT | 19943 |
| 4766 | UGUUCGAG G UGUUGAAG | 9185 | CTTCAACA GGCTAGCTACAACGA CTCCAACA | 19944 |
| 4768 | UUCGAGGU C UUCAAGAU | 9186 | ATCTTCAA GGCTAGCTACAACGA ACCTCCAA | 19945 |
| 4775 | UGUUGAAC A UCCGAAGG | 9187 | CCTTCCCA GGCTAGCTACAACGA CTTCAACA | 19946 |
| 4784 | UGGCAAGC A UUUGCAGC | 9188 | CCTCCAAA GGCTAGCTACAACGA CCTTCCCA | 19947 |
| 4788 | AAGCAUUU C CAGGGCUG | 9189 | CACCCCTC GGCTAGCTACAACGA AAATCCTT | 19948 |
| 4793 | UUUCCAGG C CUGACUCU | 9190 | ACACTCAG GGCTAGCTACAACGA CCTGCAAA | 19949 |
| 4798 | AGGGCUGA C UCUAUCCA | 9191 | TCCATACA GGCTAGCTACAACGA TCACCCCT | 19950 |
| 4802 | CUCACUCU A UCCAACAC | 9192 | CTCTTCCA GGCTAGCTACAACGA ACACTCAC | 19951 |
| 4811 | UCCAACAC C CUUUCUUU | 9193 | AAACAAAC GGCTAGCTACAACGA CTCTTCCA | 19952 |
| 4816 | CACCCUUU C UUUACCAC | 9194 | CTCCTAAA GGCTAGCTACAACGA AAACCCTC | 19953 |
| 4823 | UCUUUACC A CCUCCCUC | 9195 | CACCCACG GGCTAGCTACAACGA CCTAAACA | 19954 |
| 4825 | UUUACCAC C UCCCUCCC | 9196 | CCCACCCA GGCTAGCTACAACGA CCCCTAAA | 19955 |
| 4829 | CGACCUCC C UCCCAACC | 9197 | CCTTCCCA GGCTAGCTACAACGA CCACCTCC | 19956 |
| 4836 | CCUCCCAA C CCAACCCU | 9198 | ACCCTTCC GGCTAGCTACAACGA TTCCCACC | 19957 |
| 4841 | CAACCCAA C CCUUAACU | 9199 | ACTTAAGC GGCTAGCTACAACGA TTCCCTTC | 19958 |
| 4848 | ACCCUUAA C UCUCCAAU | 9200 | ATTCCACA GGCTAGCTACAACGA TTAACGCT | 19959 |
| 4850 | CCUUAACU C UCCAAUUC | 9201 | CAATTCCA GGCTAGCTACAACGA ACTTAACG | 19960 |
| 4855 | ACUCUCCA A UUCCCAUU | 9202 | AATCCCAA GGCTAGCTACAACGA TCCACACT | 19961 |
| 4861 | CAAUUCCC A UUCAUACA | 9203 | TCTATCAA GGCTAGCTACAACGA CCCAATTC | 19962 |
| 4865 | UCCCAUUC A UACAAAGC | 9204 | CCTTTCTA GGCTAGCTACAACGA CAATCCCA | 19963 |
| 4877 | AAACCAAC A CUAACCUU | 9205 | AACCTTAC GGCTAGCTACAACGA CTTCCTTT | 19964 |
| 4881 | CAACACUA A CCUUACCU | 9206 | ACCTAACC GGCTAGCTACAACGA TACTCTTC | 19965 |
| 4883 | ACACUAAC C UUACCUUC | 9207 | CAACCTAA GGCTAGCTACAACGA CTTACTCT | 19966 |
| 4886 | CUAACGUU A CCUUGCUU | 9208 | AAGCAAGG GGCTAGCTACAACGA AACGTTAG | 19967 |
| 4891 | GUUACCUU G CUUUGGAG | 9209 | CTCCAAAG GGCTAGCTACAACGA AAGGTAAC | 19968 |
| 4901 | UUUGGAGA G UACUGGAG | 9210 | CTCCAGTA GGCTAGCTACAACGA TCTCCAAA | 19969 |
| 4903 | UGGAGAGU A CUGGAGCC | 9211 | GGCTCCAG GCCTAGCTACAACGA ACTCTCCA | 19970 |
| 4909 | GUACUGGA G CCUGCAAA | 9212 | TTTGCAGG GGCTAGCTACAACGA TCCAGTAC | 19971 |
| 4913 | UGGAGCCU G CAAAUGCA | 9213 | TGCATTTG GGCTAGCTACAACGA AGGCTCCA | 19972 |
| 4917 | GCCUGCAA A UGCAUUGU | 9214 | ACAATGCA GGCTAGCTACAACGA TTGCAGGC | 19973 |
| 4919 | CUGCAAAU G CAUUGUGU | 9215 | ACACAATG GGCTAGCTACAACGA ATTTGCAG | 19974 |
| 4921 | GCAAAUGC A UUGUGGUG | 9216 | AAACACAA GGCTAGCTACAACGA GCATTTGC | 19975 |
| 4924 | AAUGCAUU G UGUUUGCU | 9217 | AGCAAACA GGCTAGCTACAACGA AATGCATT | 19976 |
| 4926 | UGCAUUGU G UUUGCUCU | 9218 | AGAGCAAA GGCTAGCTACAACGA ACAATGCA | 19977 |
| 4930 | UUGUGUUG G CUCUGGUG | 9219 | CACCAGAG GGCTAGCTACAACGA AAACACAA | 19978 |
| 4936 | UUGCUCUG G UGGAGGUG | 9220 | CACCTCCA GGCTAGCTACAACGA CAGAGCAA | 19979 |
| 4942 | UGGUGGAG G UGGGCAUG | 9221 | CATGCCCA GGCTAGCTACAACGA CTCCACCA | 19980 |
| 4946 | GGAGGUGG G CAUGGGGU | 9222 | ACCCCATG GGCTAGCTACAACGA CCACCTCC | 19981 |
| 4948 | AGGUGGGC A UGGGGUCU | 9223 | AGACCCCA GGCTAGCTACAACGA GCCCACCT | 19982 |
| 4953 | GGCAUGGG G UCUGUUCU | 9224 | AGAACAGA GGCTAGCTACAACGA CCCATGCC | 19983 |
| 4957 | UGGGGUCU G UUCUGAAA | 9225 | TTTCAGAA GGCTAGCTACAACGA AGACCCCA | 19984 |
| 4965 | GUUCUGAA A UGUAAAGG | 9226 | CCTTTACA GGCTAGCTACAACGA TTCAGAAC | 19985 |
| 4967 | UCUGAAAU G UAAAGGGU | 9227 | ACCCTTTA GGCTAGCTACAACGA ATTTCAGA | 19986 |
| 4974 | UGUAAAGG G UUCAGACG | 9228 | CGTCTGAA GGCTAGCTACAACGA CCTTTACA | 19987 |
| 4980 | GGGUUCAG A CGGGGUUU | 9229 | AAACCCCG GGCTAGCTACAACGA CTGAACCC | 19988 |
| 4985 | CAGACGGG G UUUCUGGU | 9230 | ACCAGAAA GGCTAGCTACAACGA CCCGTCTG | 19989 |
| 4992 | GGUUUCUG G UUUUAGAA | 9231 | TTCTAAAA GGCTAGCTACAACGA CAGAAACC | 19990 |
| 5002 | UUUAGAAG 0 UUGCGUGU | 9232 | ACACGCAA GGCTAGCTACAACGA CTTCTAAA | 19991 |
| 5005 | AGAAGGUU G CGUGUUCU | 9233 | AGAACACG GGCTAGCTACAACGA AACCTTCT | 19992 |
| 5007 | AAGGUUGC G UGUUCUUC | 9234 | GAAGAACA GGCTAGCTACAACGA GCAACCTT | 19993 |
| 5009 | GGUUGCGU G UUCUUCGA | 9235 | TCGAAGAA GGCTAGCTACAACGA ACGCAACC | 19994 |
| 5018 | UUCUUCGA G UUGGGCUA | 9236 | TAGCCCAA GGCTAGCTACAACGA TCGAAGAA | 19995 |
| 5023 | CGAGUUGG G CUAAAGUA | 9237 | TACTTTAG GGCTAGCTACAACGA CCAACTCG | 19996 |
| 5029 | GGGCUAAA G UAGAGUUC | 9238 | GAACTCTA GGCTAGCTACAACGA TTTAGCCC | 19997 |
| 5034 | AAAGUAGA G UUCGUUGU | 9239 | ACAACGAA GGCTAGCTACAACGA TCTACTTT | 19998 |
| 5038 | UAGAGUUC G UUGUGCUG | 9240 | CAGCACAA GGCTAGCTACAACGA GAACTCTA | 19999 |
| 5041 | AGUUCGUU G UGCUGUUU | 9241 | AAACAGCA GGCTAGCTACAACGA AACGAACT | 20000 |
| 5043 | UUCGUUGU G CUGUUUCU | 9242 | AGAAACAG GGCTAGCTACAACGA ACAACGAA | 20001 |
| 5046 | GUUGUGCU G UUUCUGAC | 9243 | GTCAGAAA GGCTAGCTACAACGA AGCACAAC | 20002 |
| 5053 | UGGUUCUG A CUCCUAAU | 9244 | ATTAGGAG GGCTAGCTACAACGA CAGAAACA | 20003 |
| 5060 | GACUCCUA A UGAGAGUU | 9245 | AACTCTCA GGCTAGCTACAACGA TAGGAGTC | 20004 |
| 5066 | UAAUGAGA G UUCCUUCC | 9246 | GGAAGGAA GGCTAGCTACAACGA TCTCATTA | 20005 |
| 5077 | CCUUCCAG A CCGUUAGC | 9247 | GCTAACGG GGCTAGCTACAACGA CTGGAAGG | 20006 |
| 5080 | UCCAGACC G UUAGCUGU | 9248 | ACAGCTAA GGCTAGCTACAACGA GGTCTGGA | 20007 |
| 5084 | GACCGUUA G CUGUCUCC | 9249 | GGAGACAG GGCTAGCTACAACGA TAACGGTC | 20008 |
| 5087 | CGUUAGCU G UCUCCUUG | 9250 | CAAGGAGA GGCTAGCTACAACGA AGCTAACG | 20009 |
| 5095 | GUCUCCUU G CCAAGCCC | 9251 | GGGCTTGG GGCTAGCTACAACGA AAGGAGAC | 20010 |
| 5100 | CUUGCCAA G CCCCAGGA | 9252 | TCCTGGGG GGCTAGCTACAACGA TTGGCAAG | 20011 |
| 5114 | GGAAGAAA A UGAUCCAG | 9253 | CTGCATCA GGCTAGCTACAACGA TTTCTTCC | 20012 |
| 5117 | AGAAAAUG A UGCAGCUC | 9254 | GAGCTGCA GGCTAGCTACAACGA CATTTTCT | 20013 |
| 5119 | AAAAUGAU G CAGGUCUG | 9255 | CAGAGCTG GGCTAGCTACAACGA ATCATTTT | 20014 |
| 5122 | AUGAUGCA G CUCUGGCU | 9256 | AGCCAGAG GGCTAGCTACAACGA TGCATCAT | 20015 |
| 5128 | CAGCUCUG G CUCCUUGU | 9257 | ACAAGGAC GGCTAGCTACAACGA CAGAGCTG | 20016 |
| 5135 | GGCUCCUU G UCUCCCAG | 9258 | CTGGGAGA GGCTAGCTACAACGA AAGCAGCC | 20017 |
| 5144 | UCUCCCAG G CUGAUCCU | 9259 | AGGATCAG GGCTAGCTACAACGA CTGGGAGA | 20018 |
| 5148 | CCAGGCUG A UCCUUUAU | 9260 | ATAAAGGA GGCTAGCTACAACGA CAGCCTGG | 20019 |
| 5155 | GAUCCUUU A UUCAGAAU | 9261 | ATTCTGAA GGCTAGCTACAACGA AAAGGATC | 20020 |
| 5162 | UAUUCAGA A UACCACAA | 9262 | TTGTGGTA GGCTAGCTACAACGA TCTGAATA | 20021 |
| 5164 | UUCAGAAU A CCACAAAG | 9263 | CTTTGTGG GGCTAGCTACAACGA ATTCTGAA | 20022 |
| 5167 | AGAAUACC A CAAAGAAA | 9264 | TTTCTTTG GGCTAGCTACAACGA GGTATTCT | 20023 |
| 5178 | AAGAAAGG A CAUUCAGC | 9265 | GCTGAATG GGCTAGCTACAACGA CCTTTCTT | 20024 |
| 5180 | GAAAGGAC A UUCAGCUC | 9266 | GAGCTGAA GGCTAGCTACAACGA GTCCTTTC | 20025 |
| 5185 | GACAUUCA G CUCAAGGC | 9267 | GCCTTGAG GGCTAGCTACAACGA TGAATGTC | 20026 |
| 5192 | AGCUCAAG G CUCCCUGC | 9268 | GCAGGGAG GGCTAGCTACAACGA CTTGAGCT | 20027 |
| 5199 | GGCUCCCU G CCGUGUUG | 9269 | CAACACGG GGCTAGCTACAACGA AGGGAGCC | 20028 |
| 5202 | UCCCUGCC G UGUUGAAG | 9270 | CTTCAACA GGCTAGCTACAACGA GGCAGGGA | 20029 |
| 5204 | CCUGCCGU G UUGAAGAG | 9271 | CTCTTCAA GGCTAGCTACAACGA ACGGCAGG | 20030 |
| 5212 | GUUGAAGA G UUCUGACU | 9272 | AGTCAGAA GGCTAGCTACAACGA TCTTCAAC | 20031 |
| 5218 | GAGUUCUG A CUGCACAA | 9273 | TTGTGCAG GGCTAGCTACAACGA CAGAACTC | 20032 |
| 5221 | UUCUGACU G CACAAACC | 9274 | GGTTTGTG GGCTAGCTACAACGA ACTCAGAA | 20033 |
| 5223 | CUGACUGC A CAAACCAG | 9275 | CTGGTTTG GGCTACCTACAACGA GCAGTCAG | 20034 |
| 5227 | CUGCACAA A CCAGCUUC | 9276 | GAAGCTGG GGCTAGCTACAACGA TTGTGCAG | 20035 |
| 5231 | ACAAACCA G CUUCUCGU | 9277 | ACCAGAAC GGCTAGCTACAACGA TGGTTTGT | 20036 |
| 5238 | AGCUUCUG G UUUCUUCU | 9278 | AGAAGAAA GGCTAGCTACAACGA CAGAAGCT | 20037 |
| 5250 | CUUCUGGA A UGAAUACC | 9279 | GGTATTCA GGCTAGCTACAACGA TCCAGAAG | 20038 |
| 5254 | UGGAAUGA A UACCCUCA | 9280 | TGAGGGTA GGCTAGCTACAACGA TCATTCCA | 20039 |
| 5256 | GAAUGAAU A CCCUCAUA | 9281 | TATGAGGG GGCTAGCTACAACGA ATTCATTC | 20040 |
| 5262 | AUACCCUC A UAUCUUUC | 9282 | GACAGATA GGCTAGCTACAACGA GAGGGTAT | 20041 |
| 5264 | ACCCUCAU A UCUGUCCU | 9283 | AGGACAGA GGCTAGCTACAACGA ATGAGGGT | 20042 |
| 5268 | UCAUAUCU G UCCUGAUG | 9284 | CATCAGGA GGCTAGCTACAACGA AGATATGA | 20043 |
| 5274 | CUGUCCUG A UGUGAUAU | 9285 | ATATCACA GGCTAGCTACAACGA CAGGACAG | 20044 |
| 5276 | GUCCUGAU C UGAUAUGU | 9286 | ACATATCA GGCTAGCTACAACGA ATCAGGAC | 20045 |
| 5279 | CUGAUGUG A CAUGUCUG | 9287 | CAGACATA GGCTAGCTACAACGA CACATCAG | 20046 |
| 5281 | GAUGUGAU A UGUCUGAG | 9288 | CTCAGACA GGCTAGCTACAACGA ATCACATC | 20047 |
| 5283 | UGUGAUAU G UCUGAGAC | 9289 | GTCTCAGA GGCTAGCTACAACGA ATATCACA | 20048 |
| 5290 | UGUCUGAG A CUGAAUGC | 9290 | GCATTCAG GGCTAGCTACAACGA CTCAGACA | 20049 |
| 5295 | GAGACUGA A UGCGGCAG | 9291 | CTCCCGCA GGCTAGCTACAACGA TCAGTCTC | 20050 |
| 5297 | GACUGAAU G CGGGAGGU | 9292 | ACCTCCCG GGCTAGCTACAACGA ATTCAGTC | 20051 |
| 5304 | UGCGGGAG G UUCAAUGU | 9293 | ACATTGAA GGCTAGCTACAACGA CTCCCGCA | 20052 |
| 5309 | GAGGUUCA A UGUGAAGC | 9294 | GCTTCACA GGCTAGCTACAACGA TGAACCTC | 20053 |
| 5311 | GGUUCAAU C UGAAGCUC | 9295 | CAGCTTCA GGCTAGCTACAACGA ATTGAACC | 20054 |
| 5316 | AAUCUCAA G CUGUGUGU | 9296 | ACACACAG GGCTAGCTACAACGA TTCACATT | 20055 |
| 5319 | GUGAAGCU G UGUGUGGU | 9297 | ACCACACA GGCTAGCTACAACGA AGCTTCAC | 20056 |
| 5321 | GAAGCUGU G UGUGGUGU | 9298 | ACACCACA GGCTAGCTACAACGA ACAGCTTC | 20057 |
| 5323 | ACCUCUGU G UGGUGUCA | 9299 | TGACACCA GGCTAGCTACAACGA ACACAGCT | 20058 |
| 5326 | UGUGUGUG C UCUCAAAG | 9300 | CTTTGACA GGCTAGCTACAACGA CACACACA | 20059 |
| 5328 | UGUGUGGU G UCAAAGUU | 9301 | AACTTTGA GGCTAGCTACAACGA ACCACACA | 20060 |
| 5334 | GUCUCAAA G UUUCAGGA | 9302 | TCCTGAAA GGCTAGCTACAACGA TTTGACAC | 20061 |
| 5346 | CAGGAAGG A UUUUACCC | 9303 | GGGTAAAA GGCTAGCTACAACGA CCTTCCTG | 20062 |
| 5351 | AGGAUUUU A CCCUUUUG | 9304 | CAAAAGGG GGCTAGCTACAACGA AAAATCCT | 20063 |
| 5359 | ACCCUUUU C UUCUUCCC | 9305 | GCGAAGAA GGCTAGCTACAACGA AAAAGGGT | 20064 |
| 5371 | UUCCCCCU G UCCCCAAC | 9306 | GTTGGGGA GGCTAGCTACAACGA AGGGGGAA | 20065 |
| 5378 | UGUCCCCA A CCCACUCU | 9307 | AGAGTGGG GGCTAGCTACAACGA TGGGGACA | 20066 |
| 5382 | CCCAACCC A CUCUCACC | 9308 | GGTGAGAG GGCTAGCTACAACGA GGGTTGGG | 20067 |
| 5388 | CCACUCUC A CCCCGCAA | 9309 | TTGCGGGG GGCTAGCTACAACGA GAGAGTGG | 20068 |
| 5393 | CUCACCCC G CAACCCAU | 9310 | ATGGGTTG GGCTAGCTACAACGA GGGGTGAG | 20069 |
| 5396 | ACCCCGCA A CCCAUCAG | 9311 | CTGATGGG GGCTAGCTACAACGA TGCGGGGT | 20070 |
| 5420 | GUUAUUUG G CCUCUACU | 9317 | AGTAGAGG GGCTAGCTACAACGA CAAATAAC | 20076 |
| 5426 | UGGCCUCU A CUCCAGUA | 9318 | TACTGGAG GGCTAGCTACAACGA AGAGGCCA | 20077 |
| 5432 | CUACUCCA G UAAACCUG | 9319 | CAGGTTTA GGCTAGCTACAACGA TGGAGTAG | 20078 |
| 5436 | UCCAGUAA A CCUGAUUG | 9320 | CAATCAGG GGCTAGCTACAACGA TTACTGGA | 20079 |
| 5441 | UAAACCUG A UUGGGUUU | 9321 | AAACCCAA GGCTAGCTACAACGA CAGGTTTA | 20080 |
| 5446 | CUGAUUGG G UUUGUUCA | 9322 | TGAACAAA GGCTAGCTACAACGA CCAATCAG | 20081 |
| 5450 | UUGGGUUU G UUCACUCU | 9323 | AGAGTGAA GGCTAGCTACAACGA AAACCCAA | 20082 |
| 5454 | GUUUGUUC A CUCUCUGA | 9324 | TCAGAGAG GGCTAGCTACAACGA GAACAAAC | 20083 |
| 5463 | CUCUCUGA A UGAUUAUU | 9325 | AATAATCA GGCTAGCTACAACGA TCAGAGAG | 20084 |
| 5466 | UCUGAAUG A UUAUUAGC | 9326 | GCTAATAA GGCTAGCTACAACGA CATTCAGA | 20085 |
| 5469 | GAAUGAUU A UUAGCCAG | 9327 | CTGGCTAA GGCTAGCTACAACGA AATCATTC | 20086 |
| 5473 | GAUUAUUA G CCAGACUU | 9328 | AAGTCTGG GGCTAGCTACAACGA TAATAATC | 20087 |
| 5478 | UUAGCCAG A CUUCAAAA | 9329 | TTTTGAAG GGCTAGCTACAACGA CTGGCTAA | 20088 |
| 5486 | ACUUCAAA A UUAUUUUA | 9330 | TAAAATAA GGCTAGCTACAACGA TTTGAAGT | 20089 |
| 5489 | UCAAAAUU A UUUUAUAG | 9331 | CTATAAAA GGCTAGCTACAACGA AATTTTGA | 20090 |
| 5494 | AUUAUUUU A UAGCCCAA | 9332 | TTGGGCTA GGCTAGCTACAACGA AAAATAAT | 20091 |
| 5497 | AUUUUAUA G CCCAAAUU | 9333 | AATTTGGG GGCTAGCTACAACGA TATAAAAT | 20092 |
| 5503 | UAGCCCAA A UUAUAACA | 9334 | TGTTATAA GGCTAGCTACAACGA TTGGGCTA | 20093 |
| 5506 | CCCAAAUU A UAACAUCU | 9335 | AGATGTTA GGCTAGCTACAACGA AATTTGGG | 20094 |
| 5509 | AAAUUAUA A CAUCUAUU | 9336 | AATAGATG GGCTAGCTACAACGA TATAATTT | 20095 |
| 5511 | AUUAUAAC A UCUAUUGU | 9337 | ACAATAGA GGCTAGCTACAACGA GTTATAAT | 20096 |
| 5515 | UAACAUCU A UUGUAUUA | 9338 | TAATACAA GGCTAGCTACAACGA AGATGTTA | 20097 |
| 5518 | CAUCUAUU G UAUUAUUU | 9339 | AAATAATA GGCTAGCTACAACGA AATAGATG | 20098 |
| 5520 | UCUAUUGU A UUAUUUAG | 9340 | CTAAATAA GGCTAGCTACAACGA ACAATAGA | 20099 |
| 5523 | AUUGUAUU A UUUAGACU | 9341 | AGTCTAAA GGCTAGCTACAACGA AATACAAT | 20100 |
| 5529 | UUAUUUAG A CUUUUAAC | 9342 | GTTAAAAG GGCTAGCTACAACGA CTAAATAA | 20101 |
| 5536 | GACUUUUA A CAUAUAGA | 9343 | TCTATATG GGCTAGCTACAACGA TAAAAGTC | 20102 |
| 5538 | CUUUUAAC A UAUAGAGC | 9344 | GCTCTATA GGCTAGCTACAACGA GTTAAAAG | 20103 |
| 5540 | UUUAACAU A UAGAGCUA | 9345 | TAGCTCTA GGCTAGCTACAACGA ATGTTAAA | 20104 |
| 5545 | CAUAUAGA G CUAUUUCU | 9346 | AGAAATAG GGCTAGCTACAACGA TCTATATG | 20105 |
| 5548 | AUAGAGCU A UUUCUACU | 9347 | AGTAGAAA GGCTAGCTACAACGA AGCTCTAT | 20106 |
| 5554 | CUAUUUCU A CUGAUUUU | 9348 | AAAATCAG GGCTAGCTACAACGA AGAAATAG | 20107 |
| 5558 | UUCUACUG A UUUUUGCC | 9349 | GGCAAAAA GGCTAGCTACAACGA CAGTAGAA | 20108 |
| 5564 | UGAUUUUU G CCCUUGUU | 9350 | AACAAGGG GGCTAGCTACAACGA AAAAATCA | 20109 |
| 5570 | UUGCCCUU G UUCUGUCC | 9351 | GGACAGAA GGCTAGCTACAACGA AAGGGCAA | 20110 |
| 5575 | CUUGUUCU G UCCUUUUU | 9352 | AAAAAGGA GGCTAGCTACAACGA AGAACAAG | 20111 |
| 5597 | AAAAGAAA A UGUGUUUU | 9353 | AAAACACA GGCTAGCTACAACGA TTTCTTTT | 20112 |
| 5599 | AAGAAAAU G UGUUUUUU | 9354 | AAAAAACA GGCTAGCTACAACGA ATTTTCTT | 20113 |
| 5601 | GAAAAUGU G UUUUUUGU | 9355 | ACAAAAAA GGCTAGCTACAACGA ACATTTTC | 20114 |
| 5608 | UGUUUUUU G UUUGGUAC | 9356 | GTACCAAA GGCTAGCTACAACGA AAAAAACA | 20115 |
| 5613 | UUUGUUUG G UACCAUAG | 9357 | CTATGGTA GGCTAGCTACAACGA CAAACAAA | 20116 |
| 5615 | UGUUUGGU A CCAUAGUG | 9358 | CACTATGG GGCTAGCTACAACGA ACCAAACA | 20117 |
| 5618 | UUGGUACC A UAGUGUGA | 9359 | TCACACTA GGCTAGCTACAACGA GGTACCAA | 20118 |
| 5621 | GUACCAUA C UGUGAAAU | 9360 | ATTTCACA GGCTAGCTACAACGA TATGGTAC | 20119 |
| 5623 | ACCAUAGU C UGAAAUGC | 9361 | GCATTTCA GGCTAGCTACAACGA ACTATGCT | 20120 |
| 5628 | AGUCUGAA A UGCUGGCA | 9362 | TCCCACCA GGCTAGCTACAACGA TTCACACT | 20121 |
| 5630 | UGUGAAAU C CUGCGAAC | 9363 | CTTCCCAG GGCTAGCTACAACGA ATTTCACA | 20122 |
| 5637 | UGCUGGCA A CAAUGACU | 9364 | ACTCATTG GGCTAGCTACAACGA TCCCAGCA | 20123 |
| 5640 | UGCGAACA A UGACUAUA | 9365 | TATACTCA GGCTAGCTACAACGA TGTTCCCA | 20124 |
| 5643 | CAACAAUC A CUAUAACA | 9366 | TCTTATAC GGCTAGCTACAACGA CATTCTTC | 20125 |
| 5646 | CAAUCACU A UAACACAU | 9367 | ATCTCTTA GGCTAGCTACAACGA ACTCATTG | 20126 |
| 5651 | ACUAUAAC A CAUGCUAU | 9368 | ATACCATG GGCTAGCTACAACGA CTTATAGT | 20127 |
| 5653 | UAUAAGAC A UGCUAUGC | 9369 | CCATAGCA GGCTAGCTACAACGA CTCTTATA | 20128 |
| 5655 | UAAGACAU G CUAUGCCA | 9370 | TGCCATAC GGCTAGCTACAACGA ATCTCTTA | 20129 |
| 5658 | GACAUGCU A UGGCACAU | 9371 | ATCTGCCA GGCTAGCTACAACGA ACCATGTC | 20130 |
| 5661 | AUGCUAUG G CACAUAUA | 9372 | TATATCTG GGCTAGCTACAACGA CATACCAT | 20131 |
| 5663 | CCUAUCGC A CAUAUAUU | 9373 | AATATATC GGCTAGCTACAACGA CCCATAGC | 20132 |
| 5665 | UAUGGCAC A UAUAUUUA | 9374 | TAAATATA GGCTAGCTACAACGA GTCCCATA | 20133 |
| 5667 | UGCCACAU A UAUUUAUA | 9375 | TATAAATA GGCTAGCTACAACGA ATGTGCCA | 20134 |
| 5669 | GCACAUAU A UUUAUAGU | 9376 | ACTATAAA GGCTAGCTACAACGA ATATGTCC | 20135 |
| 5673 | AUAUAUUU A UACUCUCU | 9377 | ACACACTA GGCTAGCTACAACGA AAATATAT | 20136 |
| 5676 | UAUUAUUA G UCUGUUUA | 9378 | TAAACACA GGCTAGCTACAACGA TATAAATA | 20137 |
| 5680 | UAUACUCU G UUUAUCUA | 9379 | TACATAAA GGCTAGCTACAACGA AGACTATA | 20138 |
| 5684 | GUCUGUUU A UCUACAAA | 9380 | TTTCTACA GGCTAGCTACAACGA AAACACAC | 20139 |
| 5686 | CUGUUUAU G UAGAAACA | 9381 | TCTTTCTA GGCTAGCTACAACGA ATAAACAC | 20140 |
| 5692 | AUCUAGAA A CAAAUCUA | 9382 | TACATTTC GGCTAGCTACAACGA TTCTACAT | 20141 |
| 5696 | AGAAACAA A UGUAAUAU | 9383 | ATATTACA GGCTAGCTACAACGA TTGTTTCT | 20142 |
| 5698 | AAACAAAU G UAAUAUAU | 9384 | ATATATTA GGCTAGCTACAACGA ATTTGTTT | 20143 |
| 5701 | CAAAUGUA A UAUAUUAA | 9385 | TTAATATA GGCTAGCTACAACGA TACATTTC | 20144 |
| 5703 | AAUCUAAU A UAUUAAAC | 9386 | CTTTAATA GGCTAGCTACAACGA ATTACATT | 20145 |
| 5705 | UGUAAUAU A UUAAACCC | 9387 | GGCTTTAA GGCTAGCTACAACGA ATATTACA | 20146 |
| 5711 | AUAUUAAA C CCUUAUAU | 9388 | ATATAAGC GGCTAGCTACAACGA TTTAATAT | 20147 |
| 5716 | AAAGCCUU A UAUAUAAU | 9389 | ATTATATA GGCTAGCTACAACGA AAGCCTTT | 20148 |
| 5718 | ACCCUUAU A UAUAAUGA | 9390 | TCATTATA GGCTAGCTACAACGA ATAACGCT | 20149 |
| 5720 | CCUUAUAU A UAAUCAAC | 9391 | GTTCATTA GGCTAGCTACAACGA ATATAACG | 20150 |
| 5723 | UAUAUAUA A UGAACUUU | 9392 | AAACTTCA GGCTAGCTACAACGA TATATATA | 20151 |
| 5727 | UAUAAUGA A CUUUGUAC | 9393 | CTACAAAG GGCTAGCTACAACGA TCATTATA | 20152 |
| 5732 | UCAACUUU C UACUAUUC | 9394 | GAATACTA GGCTAGCTACAACGA AAACTTCA | 20153 |
| 5734 | AACUUUGU A CUACUCAC | 9395 | CTGAATAG GGCTAGCTACAACGA ACAAACTT | 20154 |
| 5737 | UUUCUACU A UUCACAUU | 9396 | AATGTGAA GGCTAGCTACAACGA ACTACAAA | 20155 |
| 5741 | UACUAUUC A CAUUUUCU | 9397 | ACAAAATC GGCTAGCTACAACGA GAATACTA | 20156 |
| 5743 | CUAUUCAC A UUUUCUAU | 9398 | ATACAAAA GGCTAGCTACAACGA CTCAATAC | 20157 |
| 5748 | CACAUUUU C UAUCACUA | 9399 | TACTCATA GGCTAGCTACAACGA AAAATCTC | 20158 |
| 5750 | CAUUUUCU A UCACUAUU | 9400 | AATACTCA GGCTAGCTACAACGA ACAAAATC | 20159 |
| 5754 | UUCUAUCA C UAUUAUCU | 9401 | ACATAATA GGCTAGCTACAACGA TCATACAA | 20160 |
| 5756 | CUAUCACU A UUAUCUAC | 9402 | CTACATAA GGCTAGCTACAACGA ACTCATAC | 20161 |
| 5759 | UCACUAUU A UCUACCAU | 9403 | ATCCTACA GGCTAGCTACAACGA AATACTCA | 20162 |
| 5761 | AGUAUUAU C UAGCAUAA | 9404 | TTATCCTA GGCTAGCTACAACGA ATAATACT | 20163 |
| 5764 | AUUAUGUA C CAUAACAA | 9405 | TTCTTATC GGCTAGCTACAACGA TACATAAT | 20164 |
| 5766 | UAUGUAGC A UAACAAAG | 9406 | CTTTGTTA GGCTAGCTACAACGA GCTACATA | 20165 |
| 5769 | GUAGCAUA A CAAAGGUC | 9407 | CACCTTTC GGCTAGCTACAACGA TATGCTAC | 20166 |
| 5775 | UAACAAAG G UCAUAAUG | 9408 | CATTATGA GGCTAGCTACAACGA CTTTGTTA | 20167 |
| 5778 | CAAACGUC A UAAUGCUU | 9409 | AACCATTA GGCTAGCTACAACCA GACCTTTG | 20168 |
| 5781 | AGGUCAUA A UGCUUUCA | 9410 | TCAAAGCA GGCTAGCTACAACGA TATGACCT | 20169 |
| 5783 | GUCAUAAU G CUUUCAGC | 9411 | CCTGAAAC GGCTAGCTACAACGA ATTATGAC | 20170 |
| 5790 | UGCUUUCA C CAAUUCAU | 9412 | ATCAATTC GGCTAGCTACAACGA TGAAACCA | 20171 |
| 5793 | UUUCAGCA A UUGAUGUC | 9413 | GACATCAA GGCTAGCTACAACGA TCCTGAAA | 20172 |
| 5797 | ACCAAUUG A UGUCAUUU | 9414 | AAATGACA GGCTAGCPACAACGA CAATTCCT | 20173 |
| 5799 | CAAUUGAU C UCAUUUUA | 9415 | TAAAATGA GGCTAGCTACAACGA ATCAATTC | 20174 |
| 5802 | UUCAUGUC A UUUUAUUA | 9416 | TAATAAAA GGCTAGCTACAACGA CACATCAA | 20175 |
| 5807 | CUCAUUUU A UUAAAGAA | 9417 | TTCTTTAA GGCTAGCTACAACGA AAAATCAC | 20176 |
| 5815 | AUUAAAGA A CAUUGAAA | 9418 | TTTCAATC GGCTAGCTACAACGA TCTTTAAT | 20177 |
| 5817 | UAAACAAC A UUGAAAAA | 9419 | TTTTTCAA GGCTAGCTACAACGA CTTCTTTA | 20178 |

Input Sequence = AF035121.
Cut Site = R/Y
Arm Length = 8.
Core Sequence = GGCTAGCTACAACGA
AF035121 (Homo sapiens KDR/flk-1 protein mRNA, complete cds.; Acc# AF035121; 5830 bp)

TABLE XXIII
Human KDR Zinzyme and Substrate Sequence

| | | Seq ID | | Seq ID |
|---|---|---|---|---|
| Pos | Substrate | No | Zinzyme | No |
| 25 | CCGGGAGA G CGGUCAGU | 8365 | ACUGACCG GCCGAAAGGCGAGUGAGGUCU UCUCCCGG | 20179 |
| 28 | GGAGAGCG G UCAGUGUG | 8366 | CACACUGA GCCGAAAGGCGAGUGAGGUCU CGCUCUCC | 20180 |
| 32 | AGCGGUCA G UGUGUGGU | 8367 | ACCACACA GCCGAAAGGCGAGUGAGGUCU UGACCGCU | 20181 |
| 34 | CGGUCAGU G UGUGGUCG | 8368 | CGACCACA GCCGAAAGGCGAGUGAGGUCU ACUGACCG | 20182 |
| 36 | GUCAGUGU G UGGUCCCU | 8369 | AGCGACCA GCCGAAAGGCGAGUGAGGUCU ACACUGAC | 20183 |
| 39 | AGUGUGUG G UCGCUGCG | 8370 | CGCAGCGA GCCGAAAGGCGAGUGAGGUCU CACACACU | 20184 |
| 42 | GUGUGGUC G CUGCGUUU | 8371 | AAACGCAG GCCGAAAGGCGAGUGAGGUCU CACCACAC | 20185 |
| 45 | UGGUCGCU G CGUUUCCU | 8372 | AGGAAACG GCCGAAAGGCGAGUGAGGUCU AGCGACCA | 20186 |
| 47 | GUCGCUGC G UUUCCUCU | 8373 | AGACGAAA GCCGAAAGGCGAGUGAGGUCU GCAGCGAC | 20187 |
| 56 | UUUCCUCU G CCUGCGCC | 8374 | GGCGCAGG GCCGAAAGGCGAGUGAGGUCU AGAGGAAA | 20188 |
| 60 | CUCUGCCU G CGCCGGGC | 8375 | GCCCGGCG GCCGAAAGGCGAGUGAGGUCU AGGCAGAG | 20189 |
| 62 | CUGCCUGC G CCGGGCAU | 8376 | AUGCCCGG GCCGAAAGGCGAGUGAGGUCU GCAGGCAG | 20190 |
| 67 | UGCGCCGG G CAUCACUU | 8377 | AAGUGAUG GCCGAAAGGCGAGUGAGGUCU CCGGCGCA | 20191 |
| 76 | CAUCACUU G CGCGCCGC | 8380 | GCGGCGCG GCCGAAAGGCGAGUGAGGUCU AAGUGAUG | 20192 |
| 78 | UCACUUGC G CGCCGCAG | 8381 | CUGCGGCG GCCGAAAGGCGAGUGAGGUCU GCAAGUGA | 20193 |
| 80 | ACUUGCGC G CCGCAGAA | 8382 | UUCUGCGG GCCGAAAGGCGAGUGAGGUCU GCGCAAGU | 20194 |
| 83 | UGCGCGCC G CAGAAAGU | 8383 | ACUUUCUG GCCGAAAGGCGAGUGAGGUCU GGCGCGCA | 20195 |
| 90 | CGCAGAAA G UCCGUCUG | 8384 | CAGACGGA GCCGAAAGGCGAGUGAGGUCU UUUCUGCG | 20196 |
| 94 | GAAAGUCC G UCUGGCAG | 8385 | CUGCCAGA GCCGAAAGGCGAGUGAGGUCU GGACUUUC | 20197 |
| 99 | UCCGUCUG G CAGCCUGG | 8386 | CCAGGCUG GCCGAAAGGCGAGUGAGGUCU CAGACGGA | 20198 |
| 102 | GUCUGGCA G CCUGGAUA | 8387 | UAUCCAGG GCCGAAAGGCGAGUGAGGUCU UGCCAGAC | 20199 |
| 124 | UCCUACCG G CACCCGCA | 8391 | UGCGGGUG GCCGAAAGGCGAGUGAGGUCU CGGUAGGA | 20200 |
| 130 | CGGCACCC G CAGACGCC | 8393 | GGCGUCUG GCCGAAAGGCGAGUGAGGUCU GGGUGCCG | 20201 |
| 136 | CCGCAGAC G CCCCUGCA | 8395 | UGCAGGGG GCCGAAAGGCGAGUGAGGUCU GUCUGCGG | 20202 |
| 142 | ACGCCCCU G CAGCCGCC | 8396 | GGCGGCUG GCCGAAAGGCGAGUGAGGUCU AGGGGCGU | 20203 |
| 145 | CCCCUGCA G CCGCCGGU | 8397 | ACCGGCGG GCCGAAAGGCGAGUGAGGUCU UGCAGGGG | 20204 |
| 148 | CUGCAGCC G CCGGUCGG | 8398 | CCGACCGG GCCGAAAGGCGAGUGAGGUCU GGCUGCAG | 20205 |
| 152 | AGCCGCCG G UCGGCGCC | 8399 | GGCGCCGA GCCGAAAGGCGAGUGAGGUCU CGGCGGCU | 20206 |
| 156 | GCCGGUCG G CGCCCGGG | 8400 | CCCGGGCG GCCGAAAGGCGAGUGAGGUCU CGACCGGC | 20207 |
| 158 | CGGUCGGC G CCCGGGCU | 8401 | AGCCCGGG GCCGAAAGGCGAGUGAGGUCU GCCGACCG | 20208 |
| 164 | GCGCCCGG G CUCCCUAG | 8402 | CUAGGGAG GCCGAAAGGCGAGUGAGGUCU CCGGGCGC | 20209 |
| 172 | GCUCCCUA G CCCUGUGC | 8403 | GCACAGGG GCCGAAAGGCGAGUGAGGUCU UAGGGAGC | 20210 |
| 177 | CUAGCCCU G UGCGCUCA | 8404 | UGAGCGCA GCCGAAAGGCGAGUGAGGUCU AGGGCUAG | 20211 |
| 179 | AGCCCUGU G CGCUCAAC | 8405 | GUUGAGCG GCCGAAAGGCGAGUGAGGUCU ACAGGGCU | 20212 |
| 181 | CCCUGUGC G CUCAACUG | 8406 | CAGUUGAG GCCGAAAGGCGAGUGAGGUCU GCACAGGG | 20213 |
| 189 | GCUCAACU G UCCUGCGC | 8408 | GCGCAGGA GCCGAAAGGCGAGUGAGGUCU AGUUGAGC | 20214 |
| 194 | ACUGUCCU G CGCUGCGG | 8409 | CCGCAGCG GCCGAAAGGCGAGUGAGGUCU AGGACAGU | 20215 |
| 196 | UGUCCUGC G CUGCGGGG | 8410 | CCCCGCAG GCCGAAAGGCGAGUGAGGUCU GCAGGACA | 20216 |
| 199 | CCUGCGCU G CGGGGUGC | 8411 | GCACCCCG GCCGAAAGGCGAGUGAGGUCU AGCGCAGG | 20217 |
| 204 | GCUGCGGG G UGCCGCGA | 8412 | UCGCGGCA GCCGAAAGGCGAGUGAGGUCU CCCGCAGC | 20218 |
| 206 | UGCGGGGU G CCGCGAGU | 8413 | ACUCGCGG GCCGAAAGGCGAGUGAGGUCU ACCCCGCA | 20219 |
| 209 | GGGGUGCC G CGAGUUCC | 8414 | GGAACUCG GCCGAAAGGCGAGUGAGGUCU GGCACCCC | 20220 |
| 213 | UGCCGCGA G UUCCACCU | 8415 | AGGUGGAA GCCGAAAGGCGAGUGAGGUCU UCGCGGCA | 20221 |
| 224 | CCACCUCC G CGCCUCCU | 8417 | AGGAGGCG GCCGAAAGGCGAGUGAGGUCU GGAGGUGG | 20222 |
| 226 | ACCUCCGC G CCUCCUUC | 8418 | GAAGGAGG GCCGAAAGGCGAGUGAGGUCU GCGGAGGU | 20223 |
| 244 | CUAGACAG G CGCUGGGA | 8420 | UCCCAGCG GCCGAAAGGCGAGUGAGGUCU CUGUCUAG | 20224 |
| 246 | AGACAGGC G CUGGGAGA | 8421 | UCUCCCAG GCCGAAAGGCGAGUGAGGUCU GCCUGUCU | 20225 |
| 263 | AAGAACCG G CUCCCGAG | 8423 | CUCGGGAG GCCGAAAGGCGAGUGAGGUCU CGGUUCUU | 20226 |
| 271 | GCUCCCGA G UUCUGGGC | 8424 | GCCCAGAA GCCGAAAGGCGAGUGAGGUCU UCGGGAGC | 20227 |
| 278 | AGUUCUGG G CAUUUCGC | 8425 | GCGAAAUG GCCGAAAGGCGAGUGAGGUCU CCAGAACU | 20228 |
| 285 | GGCAUUUC G CCCGGCUC | 8427 | GAGCCGGG GCCGAAAGGCGAGUGAGGUCU GAAAUGCC | 20229 |
| 290 | UUCGCCCG G CUCGAGGU | 8428 | ACCUCGAG GCCGAAAGGCGAGUGAGGUCU CGGGCGAA | 20230 |
| 297 | GGCUCGAG G UGCAGGAU | 8429 | AUCCUGCA GCCGAAAGGCGAGUGAGGUCU CUCGAGCC | 20231 |
| 299 | CUCGAGGU G CAGGAUGC | 8430 | GCAUCCUG GCCGAAAGGCGAGUGAGGUCU ACCUCGAG | 20232 |
| 306 | UGCAGGAU G CAGAGCAA | 8432 | UUGCUCUG GCCGAAAGGCGAGUGAGGUCU AUCCUGCA | 20233 |
| 311 | GAUGCAGA G CAAGGUGC | 8433 | GCACCUUG GCCGAAAGGCGAGUGAGGUCU UCUGCAUC | 20234 |
| 316 | AGAGCAAG G UGCUGCUG | 8434 | CAGCAGCA GCCGAAAGGCGAGUGAGGUCU CUUGCUCU | 20235 |
| 318 | AGCAAGGU G CUGCUGGC | 8435 | GCCAGCAG GCCGAAAGGCGAGUGAGGUCU ACCUUGCU | 20236 |
| 321 | AAGGUGCU G CUGGCCGU | 8436 | ACGGCCAG GCCGAAAGGCGAGUGAGGUCU AGCACCUU | 20237 |
| 325 | UGCUGCUG G CCGUCGCC | 8437 | GGCGACGG GCCGAAAGGCGAGUGAGGUCU CAGCAGCA | 20238 |
| 328 | UGCUGGCC G UCGCCCUG | 8438 | CAGGGCGA GCCGAAAGGCGAGUGAGGUCU GGCCAGCA | 20239 |
| 331 | UGGCCGUC G CCCUGUGG | 8439 | CCACAGGG GCCGAAAGGCGAGUGAGGUCU GACGGCCA | 20240 |
| 336 | GUCGCCCU G UGGCUCUG | 8440 | CAGAGCCA GCCGAAAGGCGAGUGAGGUCU AGGGCGAC | 20241 |
| 339 | GCCCUGUG G CUCUGCGU | 8441 | ACGCAGAG GCCGAAAGGCGAGUGAGGUCU CACAGGGC | 20242 |
| 344 | GUGGCUCU G CGUGGAGA | 8442 | UCUCCACG GCCGAAAGGCGAGUGAGGUCU AGAGCCAC | 20243 |
| 346 | GGCUCUGC G UGGAGACC | 8443 | GGUCUCCA GCCGAAAGGCGAGUGAGGUCU GCAGAGCC | 20244 |
| 358 | AGACCCGG G CCGCCUCU | 8445 | AGAGGCGG GCCGAAAGGCGAGUGAGGUCU CCGGGUCU | 20245 |
| 361 | CCCGGGCC G CCUCUGUG | 8446 | CACAGAGG GCCGAAAGGCGAGUGAGGUCU GGCCCGGG | 20246 |
| 367 | CCGCCUCU G UGGGUUUG | 8447 | CAAACCCA GCCGAAAGGCGAGUGAGGUCU AGAGGCGG | 20247 |
| 371 | CUCUGUGG G UUUGCCUA | 8448 | UAGGCAAA GCCGAAAGGCGAGUGAGGUCU CCACAGAG | 20248 |
| 375 | GUGGGUUU G CCUAGUGU | 8449 | ACACUAGG GCCGAAAGGCGAGUGAGGUCU AAACCCAC | 20249 |
| 380 | UUUGCCUA G UGUUUCUC | 8450 | GAGAAACA GCCGAAAGGCGAGUGAGGUCU UAGGCAAA | 20250 |
| 382 | UGCCUAGU G UUUCUCUU | 8451 | AAGAGAAA GCCGAAAGGCGAGUGAGGUCU ACUAGGCA | 20251 |
| 396 | CUUGAUCU G CCCAGGCU | 8453 | AGCCUGGG GCCGAAAGGCGAGUGAGGUCU AGAUCAAG | 20252 |
| 402 | CUGCCCAG G CUCAGCAU | 8454 | AUGCUGAG GCCGAAAGGCGAGUGAGGUCU CUGGGCAG | 20253 |
| 407 | CAGGCUCA G CAUACAAA | 8455 | UUUGUAUG GCCGAAAGGCGAGUGAGGUCU UGAGCCUG | 20254 |
| 436 | CAAUUAAG G CUAAUACA | 8458 | UGUAUUAG GCCGAAAGGCGAGUGAGGUCU CUUAAUUG | 20255 |
| 461 | AAUUACUU G CAGGGGAC | 8462 | GUCCCCUG GCCGAAAGGCGAGUGAGGUCU AAGUAAUU | 20256 |
| 486 | UUGGACUG G CUUUGGCC | 8466 | GGCCAAAG GCCGAAAGGCGAGUGAGGUCU CAGUCCAA | 20257 |
| 492 | UGGCUUUG G CCCAAUAA | 8467 | UUAUUGGG GCCGAAAGGCGAGUGAGGUCU CAAAGCCA | 20258 |
| 506 | UAAUCAGA G UGGCAGUG | 8470 | CACUGCCA GCCGAAAGGCGAGUGAGGUCU UCUGAUUA | 20259 |
| 509 | UCAGAGUG G CAGUGAGC | 8471 | GCUCACUG GCCGAAAGGCGAGUGAGGUCU CACUCUGA | 20260 |
| 512 | GAGUGGCA G UGAGCAAA | 8472 | UUUGCUCA GCCGAAAGGCGAGUGAGGUCU UGCCACUC | 20261 |
| 516 | GGCAGUGA G CAAAGGGU | 8473 | ACCCUUUG GCCGAAAGGCGAGUGAGGUCU UCACUGCC | 20262 |
| 523 | AGCAAAGG G UGGAGGUG | 8474 | CACCUCCA GCCGAAAGGCGAGUGAGGUCU CCUUUGCU | 20263 |
| 529 | GGGUGGAG G UGACUGAG | 8475 | CUCAGUCA GCCGAAAGGCGAGUGAGGUCU CUCCACCC | 20264 |
| 537 | GUGACUGA G UGCAGCGA | 8477 | UCGCUGCA GCCGAAAGGCGAGUGAGGUCU UCAGUCAC | 20265 |
| 539 | GACUGAGU G CAGCGAUG | 8478 | CAUCGCUG GCCGAAAGGCGAGUGAGGUCU ACUCAGUC | 20266 |
| 542 | UGAGUGCA G CGAUGGCC | 8479 | GGCCAUCG GCCGAAAGGCGAGUGAGGUCU UGCACUCA | 20267 |
| 548 | CAGCGAUG G CCUCUUCU | 8481 | AGAAGAGG GCCGAAAGGCGAGUGAGGUCU CAUCGCUG | 20268 |
| 557 | CCUCUUCU G UAAGACAC | 8482 | GUGUCUUA GCCGAAAGGCGAGUGAGGUCU AGAAGAGG | 20269 |
| 580 | UUCCAAAA G UGAUCGGA | 8485 | UCCGAUCA GCCGAAAGGCGAGUGAGGUCU UUUUGGAA | 20270 |
| 601 | ACACUGGA G CCUACAAG | 8489 | CUUGUAGG GCCGAAAGGCGAGUGAGGUCU UCCAGUGU | 20271 |
| 609 | GCCUACAA G UGCUUCUA | 8490 | UAGAAGCA GCCGAAAGGCGAGUGAGGUCU UUGUAGGC | 20272 |
| 611 | CUACAAGU G CUUCUACC | 8491 | GGUAGAAG GCCGAAAGGCGAGUGAGGUCU ACUUGUAG | 20273 |
| 634 | CUGACUUG G CCUCGGUC | 8494 | GACCGAGG GCCGAAAGGCGAGUGAGGUCU CAAGUCAG | 20274 |
| 640 | UGGCCUCG G UCAUUUAU | 8495 | AUAAAUGA GCCGAAAGGCGAGUGAGGUCU CGAGGCCA | 20275 |
| 649 | UCAUUUAU G UCUAUGUU | 8496 | AACAUAGA GCCGAAAGGCGAGUGAGGUCU AUAAAUGA | 20276 |
| 655 | AUGUCUAU G UUCAAGAU | 8497 | AUCUUGAA GCCGAAAGGCGAGUGAGGUCU AUAGACAU | 20277 |
| 682 | CAUUUAUU G CUUCUGUU | 8500 | AACAGAAG GCCGAAAGGCGAGUGAGGUCU AAUAAAUG | 20278 |
| 688 | UUGCUUCU G UUAGUGAC | 8501 | GUCACUAA GCCGAAAGGCGAGUGAGGUCU AGAAGCAA | 20279 |
| 692 | UUCUGUUA G UGACCAAC | 8502 | GUUGGUCA GCCGAAAGGCGAGUGAGGUCU UAACAGAA | 20280 |
| 706 | AACAUGGA G UCGUGUAC | 8505 | GUACACGA GCCGAAAGGCGAGUGAGGUCU UCCAUGUU | 20281 |
| 709 | AUGGAGUC G UGUACAUU | 8506 | AAUGUACA GCCGAAAGGCGAGUGAGGUCU GACUCCAU | 20282 |
| 711 | GGAGUCGU G UACAUUAC | 8507 | GUAAUGUA GCCGAAAGGCGAGUGAGGUCU ACGACUCC | 20283 |
| 739 | ACAAAACU G UGGUGAUU | 8511 | AAUCACCA GCCGAAAGGCGAGUGAGGUCU AGUUUUGU | 20284 |
| 742 | AAACUGUG G UGAUUCCA | 8512 | UGGAAUCA GCCGAAAGGCGAGUGAGGUCU CACAGUUU | 20285 |
| 752 | GAUUCCAU G UCUCGGGU | 8514 | ACCCGAGA GCCGAAAGGCGAGUGAGGUCU AUGGAAUC | 20286 |
| 759 | UGUCUCGG G UCCAUUUC | 8515 | CAAAUGGA GCCGAAAGGCGAGUGAGGUCU CCGAGACA | 20287 |
| 778 | AUCUCAAC G UGUCACUU | 8518 | AAGUGACA GCCGAAAGGCGAGUGAGGUCU GUUGAGAU | 20288 |
| 780 | CUCAACGU G UCACUUUG | 8519 | CAAAGUGA GCCGAAAGGCGAGUGAGGUCU ACGUUGAG | 20289 |
| 788 | GUCACUUU G UGCAAGAU | 8520 | AUCUUGCA GCCGAAAGGCGAGUGAGGUCU AAAGUGAC | 20290 |
| 790 | CACUUUGU G CAAGAUAC | 8521 | GUAUCUUG GCCGAAAGGCGAGUGAGGUCU ACAAAGUG | 20291 |
| 814 | AGAGAUUU G UUCCUGAU | 8524 | AUCAGGAA GCCGAAAGGCGAGUGAGGUCU AAAUCUCU | 20292 |
| 824 | UCCUGAUG G UAACAGAA | 8526 | UUCUGUUA GCCGAAAGGCGAGUGAGGUCU CAUCAGGA | 20293 |
| 845 | CUGGGACA G CAAGAAGG | 8530 | CCUUCUUG GCCGAAAGGCGAGUGAGGUCU UGUCCCAG | 20294 |
| 854 | CAAGAAGG G CUUUACUA | 8531 | UAGUAAAG GCCGAAAGGCGAGUGAGGUCU CCUUCUUG | 20295 |
| 869 | UAUUCCCA G CUACAUGA | 8532 | UCAUGUAG GCCGAAAGGCGAGUGAGGUCU UGGGAAUA | 20296 |
| 881 | CAUGAUCA G CUAUGCUC | 8534 | CAGCAUAG GCCGAAAGGCGAGUGAGGUCU UGAUCAUG | 20297 |
| 886 | UCAGCUAU G CUGGCAUG | 8535 | CAUGCCAG GCCGAAAGGCGAGUGAGGUCU AUAGCUGA | 20298 |
| 890 | CUAUGCUG G CAUGGUCU | 8536 | AGACCAUG GCCGAAAGGCGAGUGAGGUCU CAGCAUAG | 20299 |
| 895 | CUGGCAUG G UCUUCUGU | 8537 | ACAGAAGA GCCGAAAGGCGAGUGAGGUCU CAUGCCAG | 20300 |
| 902 | GGUCUUCU G UGAAGCAA | 8538 | UUGCUUCA GCCGAAAGGCGAGUGAGGUCU AGAAGACC | 20301 |
| 907 | UCUGUGAA G CAAAAAUU | 8539 | AAUUUUUG GCCGAAAGGCGAGUGAGGUCU UUCACAGA | 20302 |
| 926 | UGAUGAAA G UUACCAGU | 8543 | ACUGGUAA GCCGAAAGGCGAGUGAGGUCU UUUCAUCA | 20303 |
| 933 | AGUUACCA G UCUAUUAU | 8544 | AUAAUAGA GCCGAAAGGCGAGUGAGGUCU UGGUAACU | 20304 |
| 942 | UCUAUUAU G UACAUAGU | 8545 | ACUAUGUA GCCGAAAGGCGAGUGAGGUCU AUAAUAGA | 20305 |
| 949 | UGUACAUA G UUGUCGUU | 8546 | AACGACAA GCCGAAAGGCGAGUGAGGUCU UAUGUACA | 20306 |
| 952 | ACAUAGUU G UCGUUGUA | 8547 | UACAACGA GCCGAAAGGCGAGUGAGGUCU AACUAUGU | 20307 |
| 955 | UAGUUGUC G UUGUAGGG | 8548 | CCCUACAA GCCGAAAGGCGAGUGAGGUCU GACAACUA | 20308 |
| 958 | UUGUCGUU G UAGGGUAU | 8549 | AUACCCUA GCCGAAAGGCGAGUGAGGUCU AACGACAA | 20309 |
| 963 | GUUGUAGG G UAUAGGAU | 8550 | AUCCUAUA GCCGAAAGGCGAGUGAGGUCU CCUACAAC | 20310 |
| 979 | UUUAUGAU G UGGUUCUG | 8553 | CAGAACCA GCCGAAAGGCGAGUGAGGUCU AUCAUAAA | 20311 |
| 982 | AUGAUGUG G UUCUGAGU | 8554 | ACUCAGAA GCCGAAAGGCGAGUGAGGUCU CACAUCAU | 20312 |
| 989 | GGUUCUGA G UCCGUCUC | 8555 | GAGACGGA GCCGAAAGGCGAGUGAGGUCU UCAGAACC | 20313 |
| 993 | CUGAGUCC G UCUCAUGG | 8556 | CCAUGAGA GCCGAAAGGCGAGUGAGGUCU GGACUCAG | 20314 |
| 1015 | AACUAUCU G UUGGAGAA | 8559 | UUCUCCAA GCCGAAAGGCGAGUGAGGUCU AGAUAGUU | 20315 |
| 1026 | GGAGAAAA G CUUGUCUU | 8560 | AAGACAAG GCCGAAAGGCGAGUGAGGUCU UUUUCUCC | 20316 |
| 1030 | AAAAGCUU G UCUUAAAU | 8561 | AUUUAAGA GCCGAAAGGCGAGUGAGGUCU AAGCUUUU | 20317 |
| 1040 | CUUAAAUU G UACAGCAA | 8563 | UUGCUGUA GCCGAAAGGCGAGUGAGGUCU AAUUUAAG | 20318 |
| 1045 | AUUGUACA G CAAGAACU | 8564 | AGUUCUUG GCCGAAAGGCGAGUGAGGUCU UGUACAAU | 20319 |
| 1063 | AACUAAAU G UGGGGAUU | 8568 | AAUCCCCA GCCGAAAGGCGAGUGAGGUCU AUUUAGUU | 20320 |
| 1101 | UCUUCGAA G CAUCAGCA | 8573 | UGCUCAUG GCCGAAAGGCGAGUGAGGUCU UUCGAAGA | 20321 |
| 1107 | AAGCAUCA G CAUAAGAA | 8574 | UUCUUAUG GCCGAAAGGCGAGUGAGGUCU UGAUGCUU | 20322 |
| 1120 | AGAAACUU G UAAACCGA | 8576 | UCGGUUUA GCCGAAAGGCGAGUGAGGUCU AAGUUUCU | 20323 |
| 1143 | AAAACCCA G UCUGGGAG | 8580 | CUCCCAGA GCCGAAAGGCGAGUGAGGUCU UGGGUUUU | 20324 |
| 1151 | GUCUGGGA G UGAGAUGA | 8581 | UCAUCUCA GCCGAAAGGCGAGUGAGGUCU UCCCAGAC | 20325 |
| 1172 | AUUUUUGA G CACCUUAA | 8584 | UUAAGGUG GCCGAAAGGCGAGUGAGGUCU UCAAAAAU | 20326 |
| 1190 | UAUAGAUG G UGUAACCC | 8587 | GGGUUACA GCCGAAAGGCGAGUGAGGUCU CAUCUAUA | 20327 |
| 1192 | UAGAUGGU G UAACCCGG | 8588 | CCGGGUUA GCCGAAAGGCGAGUGAGGUCU ACCAUCUA | 20328 |
| 1202 | AACCCGGA G UGACCAAG | 8590 | CUUGGUCA GCCGAAAGGCGAGUGAGGUCU UCCGGGUU | 20329 |
| 1215 | CAAGGAUU G UACACCUG | 8593 | CAGGUGUA GCCGAAAGGCGAGUGAGGUCU AAUCCUUG | 20330 |
| 1223 | GUACACCU G UGCAGCAU | 8594 | AUGCUGCA GCCGAAAGGCGAGUGAGGUCU AGGUGUAC | 20331 |
| 1225 | ACACCUGU G CAGCAUCC | 8595 | GGAUGCUG GCCGAAAGGCGAGUGAGGUCU ACAGGUGU | 20332 |
| 1228 | CCUGUGCA G CAUCCAGU | 8596 | ACUGGAUG GCCGAAAGGCGAGUGAGGUCU UGCACAGG | 20333 |
| 1235 | AGCAUCCA G UGGGCUGA | 8597 | UCAGCCCA GCCGAAAGGCGAGUGAGGUCU UGGAUGCU | 20334 |
| 1239 | UCCAGUGG G CUGAUGAC | 8598 | GUCAUCAG GCCGAAAGGCGAGUGAGGUCU CCACUGGA | 20335 |
| 1259 | GAAGAACA G CACAUUUG | 8602 | CAAAUGUG GCCGAAAGGCGAGUGAGGUCU UGUUCUUC | 20336 |
| 1267 | GCACAUUU G UCAGGGUC | 8603 | GACCCUGA GCCGAAAGGCGAGUGAGGUCU AAAUGUGC | 20337 |
| 1273 | UUGUCAGG G UCCAUGAA | 8604 | UUCAUGGA GCCGAAAGGCGAGUGAGGUCU CCUGACAA | 20338 |
| 1291 | AACCUUUU G UUGCUUUU | 8606 | AAAAGCAA GCCGAAAGGCGAGUGAGGUCU AAAAGGUU | 20339 |
| 1294 | CUUUUGUU G CUUUUGGA | 8607 | UCCAAAAU GCCGAAAGGCGAGUGAGGUCU AACAAAAG | 20340 |
| 1304 | UUUUGGAA G UGGCAUGG | 8608 | CCAUGCCA GCCGAAAGGCGAGUGAGGUCU UUCCAAAA | 20341 |
| 1307 | UGGAAGUG G CAUGGAAU | 8609 | AUUCCAUG GCCGAAAGGCGAGUGAGGUCU CACUUCCA | 20342 |
| 1321 | AAUCUCUG G UGGAAGCC | 8611 | GGCUUCCA GCCGAAAGGCGAGUGAGGUCU CAGAGAUU | 20343 |
| 1327 | UGGUGGAA G CCACGGUG | 8612 | CACCGUGG GCCGAAAGGCGAGUGAGGUCU UUCCACCA | 20344 |
| 1333 | AAGCCACG G UGGGGGAG | 8613 | CUCCCCCA GCCGAAAGGCGAGUGAGGUCU CGUGGCUU | 20345 |
| 1341 | GUGGGGGA G CGUGUCAG | 8614 | CUGACACG GCCGAAAGGCGAGUGAGGUCU UCCCCCAC | 20346 |
| 1343 | GGGGGAGC G UGUCAGAA | 8615 | UUCUGACA GCCGAAAGGCGAGUGAGGUCU GCUCCCCC | 20347 |
| 1345 | GGGAGCGU G UCAGAAUC | 8616 | GAUUCUGA GCCGAAAGGCGAGUGAGGUCU ACGCUCCC | 20348 |
| 1357 | GAAUCCCU G CGAAGUAC | 8618 | GUACUUCG GCCGAAAGGCGAGUGAGGUCU AGGGAUUC | 20349 |
| 1362 | CCUGCGAA G UACCUUGG | 8619 | CCAAGGUA GCCGAAAGGCGAGUGAGGUCU UUCGCAGG | 20350 |
| 1370 | GUACCUUG G UUACCCAC | 8620 | GUGGGUAA GCCGAAAGGCGAGUGAGGUCU CAAGGUAC | 20351 |
| 1395 | AUAAAAUG G UAUAAAAA | 8623 | UUUUUAUA GCCGAAAGGCGAGUGAGGUCU CAUUUUAU | 20352 |
| 1419 | CCCCUUGA G UCCAAUCA | 8626 | UGAUUGGA GCCGAAAGGCGAGUGAGGUCU UCAAGGGG | 20353 |
| 1438 | CAAUUAAA G CGGGGCAU | 8629 | AUGCCCCG GCCGAAAGGCGAGUGAGGUCU UUUAAUUG | 20354 |
| 1443 | AAAGCGGG G CAUGUACU | 8630 | AGUACAUG GCCGAAAGGCGAGUGAGGUCU CCCGCUUU | 20355 |
| 1447 | CGGGGCAU G UACUGACG | 8631 | CGUCAGUA GCCGAAAGGCGAGUGAGGUCU AUGCCCCG | 20356 |
| 1465 | UUAUGGAA G UGAGUGAA | 8634 | UUCACUCA GCCGAAAGGCGAGUGAGGUCU UUCCAUAA | 20357 |
| 1469 | GGAAGUGA G UGAAAGAG | 8635 | CUCUUUCA GCCGAAAGGCGAGUGAGGUCU UCACUUCC | 20358 |
| 1495 | AUUACACU G UCAUCCUU | 8638 | AAGGAUGA GCCGAAAGGCGAGUGAGGUCU AGUGUAAU | 20359 |
| 1527 | AAGGAGAA G CAGAGCCA | 8640 | UGGCUCUG GCCGAAAGGCGAGUGAGGUCU UUCUCCUU | 20360 |
| 1532 | GAAGCAGA G CCAUGUGG | 8641 | CCACAUGG GCCGAAAGGCGAGUGAGGUCU UCUGCUUC | 20361 |
| 1537 | AGAGCCAU G UGGUCUCU | 8642 | AGAGACCA GCCGAAAGGCGAGUGAGGUCU AUGGCUCU | 20362 |
| 1540 | GCCAUGUG G UCUCUCUG | 8643 | CAGAGAGA GCCGAAAGGCGAGUGAGGUCU CACAUGGC | 20363 |
| 1549 | UCUCUCUG G UUGUGUAU | 8644 | AUACACAA GCCGAAAGGCGAGUGAGGUCU CAGAGAGA | 20364 |
| 1552 | CUCUGGUU G UGUAUGUC | 8645 | GACAUACA GCCGAAAGGCGAGUGAGGUCU AACCAGAG | 20365 |
| 1554 | CUGGUUGU G UAUGUCCC | 8646 | GGGACAUA GCCGAAAGGCGAGUGAGGUCU ACAACCAG | 20366 |
| 1558 | UUGUGUAU G UCCCACCC | 8647 | GGGUGGGA GCCGAAAGGCGAGUGAGGUCU AUACACAA | 20367 |
| 1574 | CCAGAUUG G UGAGAAAU | 8649 | AUUUCUCA GCCGAAAGGCGAGUGAGGUCU CAAUCUGG | 20368 |
| 1597 | UCUCUCCU G UGGAUUCC | 8652 | GGAAUCCA GCCGAAAGGCGAGUGAGGUCU AGGAGAGA | 20369 |
| 1611 | UCCUACCA G UACGGCAC | 8654 | GUGCCGUA GCCGAAAGGCGAGUGAGGUCU UGGUAGGA | 20370 |
| 1616 | CCAGUACG G CACCACUC | 8655 | GAGUGGUG GCCGAAAGGCGAGUGAGGUCU CGUACUGG | 20371 |
| 1629 | ACUCAAAC G CUGACAUG | 8657 | CAUGUCAG GCCGAAAGGCGAGUGAGGUCU GUUUGAGU | 20372 |
| 1637 | GCUGACAU G UACGGUCU | 8659 | AGACCGUA GCCGAAAGGCGAGUGAGGUCU AUGUCAGC | 20373 |
| 1642 | CAUGUACG G UCUAUGCC | 8660 | GGCAUAGA GCCGAAAGGCGAGUGAGGUCU CGUACAUG | 20374 |
| 1648 | CGGUCUAU G CCAUUCCU | 8661 | AGGAAUGG GCCGAAAGGCGAGUGAGGUCU AUAGACCG | 20375 |
| 1662 | CCUCCCCC G CAUCACAU | 8662 | AUGUGAUG GCCGAAAGGCGAGUGAGGUCU GGGGGAGG | 20376 |
| 1677 | AUCCACUG G UAUUGGCA | 8663 | UGCCAAUA GCCGAAAGGCGAGUGAGGUCU CAGUGGAU | 20377 |
| 1683 | UGGUAUUG G CAGUUGGA | 8664 | UCCAACUG GCCGAAAGGCGAGUGAGGUCU CAAUACCA | 20378 |
| 1686 | UAUUGGCA G UUGGAGGA | 8665 | UCCUCCAA GCCGAAAGGCGAGUGAGGUCU UGCCAAUA | 20379 |
| 1698 | GACGAAGA G UGCGCCAA | 8666 | UUGGCGCA GCCGAAAGGCGAGUGAGGUCU UCUUCCUC | 20380 |
| 1700 | GGAAGAGU G CGCCAACG | 8667 | CGUUGGCG GCCGAAAGGCGAGUGAGGUCU ACUCUUCC | 20381 |
| 1702 | AAGAGUGC G CCAACGAG | 8668 | CUCGUUGG GCCGAAAGGCGAGUGAGGUCU GCACUCUU | 20382 |
| 1710 | GCCAACGA G CCCAGCCA | 8670 | UGGCUGGG GCCGAAAGGCGAGUGAGGUCU UCGUUGGC | 20383 |
| 1715 | CGAGCCCA G CCAAGCUG | 8671 | CAGCUUGG GCCGAAAGGCGAGUGAGGUCU UGGGCUCG | 20384 |
| 1720 | CCAGCCAA G CUGUCUCA | 8672 | UGAGACAG GCCGAAAGGCGAGUGAGGUCU UUGGCUGG | 20385 |
| 1723 | GCCAAGCU G UCUCAGUG | 8673 | CACUGAGA GCCGAAAGGCGAGUGAGGUCU AGCUUGGC | 20386 |
| 1729 | CUGUCUCA G UGACAAAC | 8674 | GUUUGUCA GCCGAAAGGCGAGUGAGGUCU UGAGACAG | 20387 |
| 1748 | AUACCCUU G UGAAGAAU | 8677 | AUUCUUCA GCCGAAAGGCGAGUGAGGUCU AAGGGUAU | 20388 |
| 1763 | AUGGAGAA G UGUGGAGG | 8679 | CCUCCACA GCCGAAAGGCGAGUGAGGUCU UUCUCCAU | 20389 |
| 1765 | GGAGAAGU G UGGAGGAC | 8680 | GUCCUCCA GCCGAAAGGCGAGUGAGGUCU ACUUCUCC | 20390 |
| 1798 | AAAUUGAA G UUAAUAAA | 8684 | UUUAUUAA GCCGAAAGGCGAGUGAGGUCU UUCAAUUU | 20391 |
| 1816 | AUCAAUUU G CUCUAAUU | 8688 | AAUUAGAG GCCGAAAGGCGAGUGAGGUCU AAAUUGAU | 20392 |
| 1843 | ACAAAACU G UAAGUACC | 8692 | GGUACUUA GCCGAAAGGCGAGUGAGGUCU AGUUUUGU | 20393 |
| 1847 | AACUGUAA G UACCCUUG | 8693 | CAAGGGUA GCCGAAAGGCGAGUGAGGUCU UUACAGUU | 20394 |
| 1855 | GUACCCUU G UUAUCCAA | 8694 | UUGGAUAA GCCGAAAGGCGAGUGAGGUCU AAGGGUAC | 20395 |
| 1864 | UUAUCCAA G CGGCAAAU | 8695 | AUUUGCCG GCCGAAAGGCGAGUGAGGUCU UUGGAUAA | 20396 |
| 1867 | UCCAAGCG G CAAAUGUG | 8696 | CACAUUUG GCCGAAAGGCGAGUGAGGUCU CGCUUGGA | 20397 |
| 1873 | CGGCAAAU G UGUCAGCU | 8698 | AGCUGACA GCCGAAAGGCGAGUGAGGUCU AUUUGCCG | 20398 |
| 1875 | GCAAAUGU G UCAGCUUU | 8699 | AAAGCUGA GCCGAAAGGCGAGUGAGGUCU ACAUUUGC | 20399 |
| 1879 | AUGUGUCA G CUUUGUAC | 8700 | GUACAAAG GCCGAAAGGCGAGUGAGGUCU UGACACAU | 20400 |
| 1884 | UCAGCUUU G UACAAAUG | 8701 | CAUUUGUA GCCGAAAGGCGAGUGAGGUCU AAAGCUGA | 20401 |
| 1892 | GUACAAAU G UGAAGCGG | 8703 | CCGCUUCA GCCGAAAGGCGAGUGAGGUCU AUUUGUAC | 20402 |
| 1897 | AAUGUGAA G CGGUCAAC | 8704 | GUUGACCG GCCGAAAGGCGAGUGAGGUCU UUCACAUU | 20403 |
| 1900 | GUGAAGGG G UCAACAAA | 8705 | UUUGUUGA GCCGAAAGGCGAGUGAGGUCU CGCUUCAC | 20404 |
| 1909 | UCAACAAA G UCGGGAGA | 8707 | UCUCCCGA GCCGAAAGGCGAGUGAGGUCU UUUGUUGA | 20405 |
| 1927 | CAGAGAGG G UGAUCUCC | 8708 | GGAGAUCA GCCGAAAGGCGAGUGAGGUCU CCUCUCUC | 20406 |
| 1942 | CCUUCCAC G UGACCAGG | 8710 | CCUGGUCA GCCGAAAGGCGAGUGAGGUCU GUGGAAGG | 20407 |
| 1952 | GACCAGGG G UCCUGAAA | 8712 | UUUCAGGA GCCGAAAGGCGAGUGAGGUCU CCCUGGUC | 20408 |
| 1968 | AUUACUUU G CAACCUGA | 8714 | UCAGGUUG GCCGAAAGGCGAGUGAGGUCU AAAGUAAU | 20409 |
| 1980 | CCUCACAU G CAGCCCAC | 8717 | GUGGGCUG GCCGAAAGGCGAGUGAGGUCU AUGUCAGG | 20410 |
| 1983 | GACAUGCA G CCCACUGA | 8718 | UCAGUGGG GCCGAAAGGCGAGUGAGGUCU UGCAUGUC | 20411 |
| 1992 | CCCACUGA G CAGGAGAG | 8719 | CUCUCCUG GCCGAAAGGCGAGUGAGGUCU UCAGUGGG | 20412 |
| 2000 | GCAGGAGA G CGUGUCUU | 8720 | AAGACACG GCCGAAAGGCGAGUGAGGUCU UCUCCUGC | 20413 |
| 2002 | AGGAGAGC G UGUCUUUG | 8721 | CAAAGACA GCCGAAAGGCGAGUGAGGUCU GCUCUCCU | 20414 |
| 2004 | GAGAGCGU G UCUUUGUG | 8722 | CACAAAGA GCCGAAAGGCGAGUGAGGUCU ACGCUCUC | 20415 |
| 2010 | GUGUCUUU G UGGUGCAC | 8723 | GUGCACCA GCCGAAAGGCGAGUGAGGUCU AAAGACAC | 20416 |
| 2013 | UCUUUGUG G UGCACUGC | 8724 | GCAGUGCA GCCGAAAGGCGAGUGAGGUCU CACAAAGA | 20417 |
| 2015 | UUUGUGGU G CACUGCAG | 8725 | CUGCAGUG GCCGAAAGGCGAGUGAGGUCU ACCACAAA | 20418 |
| 2020 | GGUGCACU G CAGACAGA | 8726 | UCUGUCUG GCCGAAAGGCGAGUGAGGUCU AGUGCACC | 20419 |
| 2034 | AGAUCUAC G UUUGAGAA | 8729 | UUCUCAAA GCCGAAAGGCGAGUGAGGUCU GUAGAUCU | 20420 |
| 2052 | CUCACAUG G UACAAGCU | 8731 | AGCUUGUA GCCGAAAGGCGAGUGAGGUCU CAUGUGAG | 20421 |
| 2058 | UGGUACAA G CUUGGCCC | 8732 | GGGCCAAG GCCGAAAGGCGAGUGAGGUCU UUGUACCA | 20422 |
| 2063 | CAAGCUUG G CCCACAGC | 8733 | GCUGUGGG GCCGAAAGGCGAGUGAGGUCU CAAGCUUG | 20423 |
| 2070 | GGCCCACA G CCUCUGCC | 8734 | GGCAGAGG GCCGAAAGGCGAGUGAGGUCU UGUGGGCC | 20424 |
| 2076 | CAGCCUCU G CCAAUCCA | 8735 | UGGAUUGG GCCGAAAGGCGAGUGAGGUCU AGAGUCUG | 20425 |
| 2086 | CAAUCCAU G UGGGAGAG | 8737 | CUCUCCCA GCCGAAAGGCGAGUGAGGUCU AUGGAUUG | 20426 |
| 2094 | GUGGGAGA G UUGCCCAC | 8738 | GUGGGCAA GCCGAAAGGCGAGUGAGGUCU UCUCCCAC | 20427 |
| 2097 | GGAGAGUU G CCCACACC | 8739 | GGUGUGGG GCCGAAAGGCGAGUGAGGUCU AACUCUCC | 20428 |
| 2107 | CCACACCU G UUUGCAAG | 8740 | CUUGCAAA GCCGAAAGGCGAGUGAGGUCU AGGUGUGG | 20429 |
| 2111 | ACCUGUUU G CAAGAACU | 8741 | AGUUCUUG GCCGAAAGGCGAGUGAGGUCU AAACAGGU | 20430 |
| 2143 | AAUUGAAU G CCACCAUG | 8746 | CAUGGUGG GCCGAAAGGCGAGUGAGGUCU AUUCAAUU | 20431 |
| 2151 | GCCACCAU G UUCUCUAA | 8747 | UUAGAGAA GCCGAAAGGCGAGUGAGGUCU AUGGUGGC | 20432 |
| 2162 | CUCUAAUA G CACAAAUG | 8749 | CAUUUGUG GCCGAAAGGCGAGUGAGGUCU UAUUAGAG | 20433 |
| 2187 | AUCAUGGA G CUUAAGAA | 8753 | UUCUUAAG GCCGAAAGGCGAGUGAGGUCU UCCAUGAU | 20434 |
| 2197 | UUAAGAAU G CAUCCUUG | 8755 | CAAGGAUG GCCGAAAGGCGAGUGAGGUCU AUUCUUAA | 20435 |
| 2205 | GCAUCCUU G CAGGACCA | 8756 | UGGUCCUG GCCGAAAGGCGAGUGAGGUCU AAGGAUGC | 20436 |
| 2224 | GAGACUAU G UCUGCCUU | 8759 | AAGGCAGA GCCGAAAGGCGAGUGAGGUCU AUAGUCUC | 20437 |
| 2228 | CUAUGUCU G CCUUGCUC | 8760 | GAGCAAGG GCCGAAAGGCGAGUGAGGUCU AGACAUAG | 20438 |
| 2233 | UCUGCCUU G CUCAAGAC | 8761 | GUCCUGAG GCCGAAAGGCGAGUGAGGUCU AAGGCAGA | 20439 |
| 2264 | AAGACAUU G CGUGGUCA | 8765 | UGACCACG GCCGAAAGGCGAGUGAGGUCU AAUGUCUU | 20440 |
| 2266 | GACAUUGC G UGGUCAGG | 8766 | CCUGACCA GCCGAAAGGCGAGUGAGGUCU GCAAUGUC | 20441 |
| 2269 | AUUGCGUG G UCAGGCAG | 8767 | CUGCCUGA GCCGAAAGGCGAGUGAGGUCU CACGCAAU | 20442 |
| 2274 | GUGGUCAG G CAGCUCAC | 8768 | GUGAGCUG GCCGAAAGGCGAGUGAGGUCU CUGACCAC | 20443 |
| 2277 | GUCAGGCA G CUCACAGU | 8769 | ACUGUGAG GCCGAAAGGCGAGUGAGGUCU UGCCUGAC | 20444 |
| 2284 | AGCUCACA G UCCUAGAG | 8770 | CUCUAGGA GCCGAAAGGCGAGUGAGGUCU UGUGAGCU | 20445 |
| 2292 | GUCCUAGA G CGUGUGGC | 8771 | GCCACACG GCCGAAAGGCGAGUGAGGUCU UCUAGGAC | 20446 |
| 2294 | CCUAGAGC G UGUGGCAC | 8772 | GUGCCACA GCCGAAAGGCGAGUGAGGUCU GCUCUAGG | 20447 |
| 2296 | UAGAGCGU G UGGCACCC | 8773 | GGGUGCCA GCCGAAAGGCGAGUGAGGUCU ACGCUCUA | 20448 |
| 2299 | AGCGUGUG G CACCCACG | 8774 | CGUGGGUG GCCGAAAGGCGAGUGAGGUCU CACACGCU | 20449 |
| 2339 | GACGACAA G UAUUGGGG | 8780 | CCCCAAUA GCCGAAAGGCGAGUGAGGUCU UUGUCGUC | 20450 |
| 2351 | UGGGGAAA G CAUCGAAG | 8781 | CUUCGAUG GCCGAAAGGCGAGUGAGGUCU UUUCCCCA | 20451 |
| 2359 | GCAUCGAA G UCUCAUGC | 8782 | GCAUGAGA GCCGAAAGGCGAGUGAGGUCU UUCGAUGC | 20452 |
| 2366 | AGUCUCAU G CACGGCAU | 8783 | AUGCCGUG GCCGAAAGGCGAGUGAGGUCU AUGAGACU | 20453 |
| 2371 | CAUGCACG G CAUCUGGG | 8784 | CCCAGAUG GCCGAAAGGCGAGUGAGGUCU CGUGCAUG | 20454 |
| 2400 | CAGAUCAU G UGGUUUAA | 8787 | UUAAACCA GCCGAAAGGCGAGUGAGGUCU AUGAUCUG | 20455 |
| 2403 | AUCAUGUG G UUUAAAGA | 8788 | UCUUUAAA GCCGAAAGGCGAGUGAGGUCU CACAUGAU | 20456 |
| 2425 | AGACCCUU G UAGAAGAC | 8792 | GUCUUCUA GCCGAAAGGCGAGUGAGGUCU AAGGGUCU | 20457 |
| 2438 | AGACUCAG G CAUUGUAU | 8794 | AUACAAUG GCCGAAAGGCGAGUGAGGUCU CUGAGUCU | 20458 |
| 2443 | CAGGCAUU G UAUUGAAG | 8795 | CUUCAAUA GCCGAAAGGCGAGUGAGGUCU AAUGCCUG | 20459 |
| 2477 | CACUAUCC G CAGAGUGA | 8799 | UCACUCUG GCCGAAAGGCGAGUGAGGUCU GGAUAGUG | 20460 |
| 2482 | UCCGCAGA G UGAGGAAG | 8800 | CUUCCUCA GCCGAAAGGCGAGUGAGGUCU UCUGCGGA | 20461 |
| 2501 | GGACGAAG G CCUCUACA | 8802 | UGUAGAGG GCCGAAAGGCGAGUGAGGUCU CUUCGUCC | 20462 |
| 2513 | CUACACCU G CCAGGCAU | 8803 | AUGCCUGG GCCGAAAGGCGAGUGAGGUCU AGGUGUAG | 20463 |
| 2518 | CCUGCCAG G CAUGCAGU | 8804 | ACUGCAUG GCCGAAAGGCGAGUGAGGUCU CUGGCAGG | 20464 |
| 2522 | CCAGGCAU G CAGUGUUC | 8805 | GAACACUG GCCGAAAGGCGAGUGAGGUCU AUGCCUGG | 20465 |
| 2525 | GGCAUGCA G UGUUCUUG | 8806 | CAAGAACA GCCGAAAGGCGAGUGAGGUCU UGCAUGCC | 20466 |
| 2527 | CAUGCAGU G UUCUUGGC | 8807 | GCCAAGAA GCCGAAAGGCGAGUGAGGUCU ACUGGAUG | 20467 |
| 2534 | UGUUCUUG G CUGUGCAA | 8808 | UUGCACAG GCCGAAAGGCGAGUGAGGUCU CAAGAACA | 20468 |
| 2537 | UCUUGGCU G UGCAAAAG | 8809 | CUUUUGCA GCCGAAAGGCGAGUGAGGUCU AGCCAAGA | 20469 |
| 2539 | UUGGCUGU G CAAAAGUG | 8810 | CACUUUUG GCCGAAAGGCGAGUGAGGUCU ACAGCCAA | 20470 |
| 2545 | GUGCAAAA G UGGAGGCA | 8811 | UGCCUCCA GCCGAAAGGCGAGUGAGGUCU UUUUGCAC | 20471 |
| 2551 | AAGUGGAG G CAUUUUUC | 8812 | GAAAAAUG GCCGAAAGGCGAGUGAGGUCU CUCCACUU | 20472 |
| 2570 | AAUAGAAG G UGCCCAGG | 8814 | CCUGGGCA GCCGAAAGGCGAGUGAGGUCU CUUCUAUU | 20473 |
| 2572 | UAGAAGGU G CCCAGGAA | 8815 | UUCCUGGG GCCGAAAGGCGAGUGAGGUCU ACCUUCUA | 20474 |
| 2608 | UUAUUCUA G UAGGCACG | 8819 | CGUGCCUA GCCGAAAGGCGAGUGAGGUCU UAGAAUAA | 20475 |
| 2612 | UCUAGUAG G CACGGCGG | 8820 | CCGCCGUG GCCGAAAGGCGAGUGAGGUCU CUACUAGA | 20476 |
| 2617 | UAGGCACG G CGGUGAUU | 8822 | AAUCACCG GCCGAAAGGCGAGUGAGGUCU CGUGCCUA | 20477 |
| 2620 | GCACGGCG G UGAUUGCC | 8823 | GGCAAUCA GCCGAAAGGCGAGUGAGGUCU CGCCGUGC | 20478 |
| 2626 | CGGUGAUU G CCAUGUUC | 8825 | GAACAUGG GCCGAAAGGCGAGUGAGGUCU AAUCACCG | 20479 |
| 2631 | AUUGCCAU G UUCUUCUG | 8826 | CAGAAGAA GCCGAAAGGCGAGUGAGGUCU AUGGCAAU | 20480 |
| 2640 | UUCUUCUG G CUACUUCU | 8827 | AGAAGUAG GCCGAAAGGCGAGUGAGGUCU CAGAAGAA | 20481 |
| 2650 | UACUUCUU G UCAUCAUC | 8828 | GAUGAUGA GCCGAAAGGCGAGUGAGGUCU AAGAAGUA | 20482 |
| 2668 | UACGGACC G UUAAGCGG | 8832 | CCGCUUAA GCCGAAAGGCGAGUGAGGUCU GGUCCGUA | 20483 |
| 2673 | ACCGUUAA G CGGGCCAA | 8833 | UUGGCCCG GCCGAAAGGCGAGUGAGGUCU UUAACGGU | 20484 |
| 2677 | UUAAGCGG G CCAAUGGA | 8834 | UCCAUUGG GCCGAAAGGCGAGUGAGGUCU CCGCUUAA | 20485 |
| 2702 | GAAGACAG G CUACUUGU | 8838 | ACAAGUAG GCCGAAAGGCGAGUGAGGUCU CUGUCUUC | 20486 |
| 2709 | UGCUACUU G UCCAUCGU | 8839 | ACGAUGGA GCCGAAAGGCGAGUGAGGUCU AAGUAGCC | 20487 |
| 2716 | UGUCCAUC G UCAUGGAU | 8840 | AUCCAUGA GCCGAAAGGCGAGUGAGGUCU GAUGGACA | 20488 |
| 2753 | UGAACAUU G UGAACGAC | 8846 | GUCGUUCA GCCGAAAGGCGAGUGAGGUCU AAUGUUCA | 20489 |
| 2763 | GAACGACU G CCUUAUGA | 8849 | UCAUAAGG GCCGAAAGGCGAGUGAGGUCU AGUCGUUC | 20490 |
| 2773 | CUUAUGAU G CCAGCAAA | 8851 | UUUGCUGG GCCGAAAGGCGAGUGAGGUCU AUCAUAAG | 20491 |
| 2777 | UGAUGCCA G CAAAUGGG | 8852 | CCCAUUUG GCCGAAAGGCGAGUGAGGUCU UGGCAUCA | 20492 |
| 2802 | AGAGACCG G CUGAAGCU | 8856 | AGCUUCAG GCCGAAAGGCGAGUGAGGUCU CGGUCUCU | 20493 |
| 2808 | CGGCUGAA G CUAGGUAA | 8857 | UUACCUAG GCCGAAAGGCGAGUGAGGUCU UUCAGCCG | 20494 |
| 2813 | GAAGCUAG G UAAGCCUC | 8858 | GAGGCUUA GCCGAAAGGCGAGUGAGGUCU CUAGCUUC | 20495 |
| 2817 | CUAGGUAA G CCUCUUGG | 8859 | CCAAGAGG GCCGAAAGGCGAGUGAGGUCU UUACCUAG | 20496 |
| 2825 | GCCUCUUG G CCGUGGUG | 8860 | CACCACGG GCCGAAAGGCGAGUGAGGUCU CAAGAGGC | 20497 |
| 2828 | UCUUGGCC G UGGUGCCU | 8861 | AGGCACCA GCCGAAAGGCGAGUGAGGUCU GGCCAAGA | 20498 |
| 2831 | UGGCCGUG G UGCCUUUG | 8862 | CAAAGGCA GCCGAAAGGCGAGUGAGGUCU CACGGCCA | 20499 |
| 2833 | GCCGUGGU G CCUUUGGC | 8863 | GCCAAAGG GCCGAAAGGCGAGUGAGGUCU ACCACGGC | 20500 |
| 2840 | UGCCUUUG G CCAAGUGA | 8864 | UCACUGGG GCCGAAAGGCGAGUGAGGUCU CAAAGGCA | 20501 |
| 2845 | UUGGCCAA G UGAUUGAA | 8865 | UUCAAUCA GCCGAAAGGCGAGUGAGGUCU UUGGCCAA | 20502 |
| 2854 | UGAUUGAA G CAGAUGCC | 8867 | GGCAUCUG GCCGAAAGGCGAGUGAGGUCU UUCAAUCA | 20503 |
| 2860 | AAGCAGAU G CCUUUGGA | 8869 | UCCAAAGG GCCGAAAGGCGAGUGAGGUCU AUCUGCUU | 20504 |
| 2881 | ACAAGACA G CAACUUGC | 8873 | GCAAGUUG GCCGAAAGGCGAGUGAGGUCU UGUCUUGU | 20505 |
| 2888 | AGCAACUU G CAGGACAG | 8875 | CUGUCCUG GCCGAAAGGCGAGUGAGGUCU AAGUUGCU | 20506 |
| 2896 | GCAGGACA G UAGCAGUC | 8877 | GACGUCUA GCCGAAAGGCGAGUGAGGUCU UGUCCUGC | 20507 |
| 2899 | GGACAGUA G CAGUCAAA | 8878 | UUUGACUG GCCGAAAGGCGAGUGAGGUCU UACUGUCC | 20508 |
| 2902 | CAGUAGCA G UCAAAAUG | 8879 | CAUUUUGA GCCGAAAGGCGAGUGAGGUCU UGCUACUG | 20509 |
| 2910 | GUCAAAAU G UUGAAAGA | 8881 | UCUUUCAA GCCGAAAGGCGAGUGAGGUCU AUUUUGAC | 20510 |
| 2923 | AAGAAGGA G CAACACAC | 8882 | GUGUGUUG GCCGAAAGGCGAGUGAGGUCU UCCUUCUU | 20511 |
| 2933 | AACACACA G UGAGCAUC | 8884 | GAUGCUCA GCCGAAAGGCGAGUGAGGUCU UGUGUGUU | 20512 |
| 2937 | CACAGUGA G CAUCGAGC | 8885 | GCUCGAUG GCCGAAAGGCGAGUGAGGUCU UCACUGUG | 20513 |
| 2944 | AGCAUCGA G CUCUCAUG | 8886 | CAUGAGAG GCCGAAAGGCGAGUGAGGUCU UCGAUGCU | 20514 |
| 2952 | GCUCUCAU G UCUGAACU | 8887 | AGUUCAGA GCCGAAAGGCGAGUGAGGUCU AUGAGAGC | 20515 |
| 2981 | UCAUAUUG G UCACCAUC | 8890 | GAUGGUGA GCCGAAAGGCGAGUGAGGUCU CAAUAUGA | 20516 |
| 2995 | AUCUCAAU G UGGUCAAC | 8892 | GUUGACCA GCCGAAAGGCGAGUGAGGUCU AUUGAGAU | 20517 |
| 2998 | UCAAUGUG G UCAACCUU | 8893 | AAGGUUGA GCCGAAAGGCGAGUGAGGUCU CACAUUGA | 20518 |
| 3011 | CCUUCUAG G UGCCUGUA | 8895 | UACAGGCA GCCGAAAGGCGAGUGAGGUCU CUAGAAGG | 20519 |
| 3013 | UUCUAGGU G CCUGUACC | 8896 | GGUACAGG GCCGAAAGGCGAGUGAGGUCU ACCUAGAA | 20520 |
| 3017 | AGGUGCCU G UACCAAGC | 8897 | GCUUGGUA GCCGAAAGGCGAGUGAGGUCU AGGCACCU | 20521 |
| 3024 | UGUACCAA G CCAGGAGG | 8898 | CCUCCUGG GCCGAAAGGCGAGUGAGGUCU UUGGUACA | 20522 |
| 3033 | CCAGGAGG G CCACUCAU | 8899 | AUGAGUGG GCCGAAAGGCGAGUGAGGUCU CCUCCUGG | 20523 |
| 3043 | CACUCAUG G UGAUUGUG | 8900 | CACAAUCA GCCGAAAGGCGAGUGAGGUCU CAUGAGUG | 20524 |
| 3049 | UGGUGAUU G UGGAAUUC | 8902 | GAAUUCCA GCCGAAAGGCGAGUGAGGUCU AAUCACCA | 20525 |
| 3059 | GGAAUUCU G CAAAUUUG | 8904 | CAAAUUUG GCCGAAAGGCGAGUGAGGUCU AGAAUUCC | 20526 |
| 3075 | GGAAACCU G UCCACUUA | 8907 | UAAGUGGA GCCGAAAGGCGAGUGAGGUCU AGGUUUCC | 20527 |
| 3092 | CCUGAGGA G CAAGAGAA | 8908 | UUCUCUUG GCCGAAAGGCGAGUGAGGUCU UCCUCAGG | 20528 |
| 3109 | AUGAAUUU G UCCCCUAC | 8911 | GUAGGGGA GCCGAAAGGCGAGUGAGGUCU AAAUUCAU | 20529 |
| 3130 | CCAAAGGG G CACGAUUC | 8913 | GAAUCGUG GCCGAAAGGCGAGUGAGGUCU CCCUUUGG | 20530 |
| 3140 | ACGAUUCC G UCAAGGGA | 8915 | UCCCUUGA GCCGAAAGGCGAGUGAGGUCU GGAAUCGU | 20531 |
| 3157 | AAGACUAC G UUGGAGCA | 8917 | UGCUCCAA GCCGAAAGGCGAGUGAGGUCU GUAGUCUU | 20532 |
| 3163 | ACGUUGGA G CAAUCCCU | 8918 | AGGGAUUG GCCGAAAGGCGAGUGAGGUCU UCCAACGU | 20533 |
| 3172 | CAAUCCCU G UGGAUCUG | 8920 | CAGAUCCA GCCGAAAGGCGAGUGAGGUCU AGGGAUUG | 20534 |
| 3186 | CUGAAACG G CGCUUGGA | 8923 | UCCAAGCG GCCGAAAGGCGAGUGAGGUCU CGUUUCAG | 20535 |
| 3188 | GAAACGGC G CUUGGACA | 8924 | UGUCCAAG GCCGAAAGGCGAGUGAGGUCU GCCGUUUC | 20536 |
| 3197 | CUUGGACA G CAUCACCA | 8926 | UGGUGAUG GCCGAAAGGCGAGUGAGGUCU UGUCCAAG | 20537 |
| 3206 | CAUCACCA G UAGCCAGA | 8927 | UCUGGCUA GCCGAAAGGCGAGUGAGGUCU UGGUGAUG | 20538 |
| 3209 | CACCAGUA G CCAGAGCU | 8928 | AGCUCUGG GCCGAAAGGCGAGUGAGGUCU UACUGGUG | 20539 |
| 3215 | UAGCCAGA G CUCAGCCA | 8929 | UGGCUGAG GCCGAAAGGCGAGUGAGGUCU UCUGGCUA | 20540 |
| 3220 | AGAGCUCA G CCAGCUCU | 8930 | AGAGCUGG GCCGAAAGGCGAGUGAGGUCU UGAGCUCU | 20541 |
| 3224 | CUCAGCCA G CUCUGGAU | 8931 | AUCCAGAG GCCGAAAGGCGAGUGAGGUCU UGGCUGAG | 20542 |
| 3235 | CUGGAUUU G UGGAGGAG | 8933 | CUCCUCCA GCCGAAAGGCGAGUGAGGUCU AAAUCCAG | 20543 |
| 3246 | GAGGAGAA G UCCCUCAG | 8934 | CUGAGGGA GCCGAAAGGCGAGUGAGGUCU UUCUCCUC | 20544 |
| 3254 | GUCCCUCA G UGAUGUAG | 8935 | CUACAUCA GCCGAAAGGCGAGUGAGGUCU UGAGGGAC | 20545 |
| 3259 | UCAGUGAU G UAGAAGAA | 8937 | UUCUUCUA GCCGAAAGGCGAGUGAGGUCU AUCACUGA | 20546 |
| 3274 | AAGAGGAA G CUCCUGAA | 8938 | UUCAGGAG GCCGAAAGGCGAGUGAGGUCU UUCCUCUU | 20547 |
| 3288 | GAAGAUCU G UAUAAGGA | 8940 | UCCUUAUA GCCGAAAGGCGAGUGAGGUCU AGAUCUUC | 20548 |
| 3312 | ACCUUGGA G CAUCUCAU | 8943 | AUGACAUG GCCGAAAGGCGAGUGAGGUCU UCCAAGGU | 20549 |
| 3323 | UCUCAUCU G UUACAGCU | 8944 | AGCUGUAA GCCGAAAGGCGAGUGAGGUCU AGAUGAGA | 20550 |
| 3329 | CUGUUACA G CUUCCAAG | 8945 | CUUGGAAG GCCGAAAGGCGAGUGAGGUCU UGUAACAG | 20551 |
| 3337 | GCUUCCAA G UGGCUAAG | 8946 | CUUAGCCA GCCGAAAGGCGAGUGAGGUCU UUGGAAGC | 20552 |
| 3340 | UCCAAGUG G CUAAGGGC | 8947 | GCCCUUAG GCCGAAAGGCGAGUGAGGUCU CACUUGGA | 20553 |
| 3347 | GGCUAAGG G CAUGGAGU | 8948 | ACUCCAUG GCCGAAAGGCGAGUGAGGUCU CCUUAGCC | 20554 |
| 3354 | GGCAUGGA G UUCUUGGC | 8949 | GCCAAGAA GCCGAAAGGCGAGUGAGGUCU UCCAUGCC | 20555 |
| 3361 | AGUUCUUG G CAUCGCGA | 8950 | UCGCGAUG GCCGAAAGGCGAGUGAGGUCU CAAGAACU | 20556 |
| 3366 | UUGGCAUC G CGAAAGUG | 8951 | CACUUUCG GCCGAAAGGCGAGUGAGGUCU GAUGCCAA | 20557 |
| 3372 | UCGCGAAA G UGUAUCCA | 8952 | UGGAUACA GCCGAAAGGCGAGUGAGGUCU UUUCGCGA | 20558 |
| 3374 | GCGAAAGU G UAUCCACA | 8953 | UGUGGAUA GCCGAAAGGCGAGUGAGGUCU ACUUUCGC | 20559 |
| 3391 | GGGACCUG G CGGCACGA | 8955 | UCGUGCCG GCCGAAAGGCGAGUGAGGUCU CAGGUCCC | 20560 |
| 3394 | ACCUGGCG G CACGAAAU | 8956 | AUUUCGUG GCCGAAAGGCGAGUGAGGUCU CGCCAGGU | 20561 |
| 3424 | AGAAGAAC G UGGUUAAA | 8959 | UUUAACCA GCCGAAAGGCGAGUGAGGUCU GUUCUUCU | 20562 |
| 3427 | AGAACGUG G UUAAAAUC | 8960 | GAUUUUAA GCCGAAAGGCGAGUGAGGUCU CACGUUCU | 20563 |
| 3437 | UAAAAUCU G UGACUUUG | 8962 | CAAAGUCA GCCGAAAGGCGAGUGAGGUCU AGAUUUUA | 20564 |
| 3446 | UGACUUUG G CUUGGCCC | 8964 | GGGCCAAG GCCGAAAGGCGAGUGAGGUCU CAAAGUCA | 20565 |
| 3451 | UUGGCUUG G CCCGGGAU | 8965 | AUCCCGGG GCCGAAAGGCGAGUGAGGUCU CAAGCCAA | 20566 |
| 3481 | CAGAUUAU G UCAGAAAA | 8969 | UUUUCUGA GCCGAAAGGCGAGUGAGGUCU AUAAUCUG | 20567 |
| 3496 | AAGGAGAU G CUCGCCUC | 8971 | GAGGCGAG GCCGAAAGGCGAGUGAGGUCU AUCUCCUU | 20568 |
| 3500 | AGAUGCUC G CCUCCCUU | 8972 | AAGGGAGG GCCGAAAGGCGAGUGAGGUCU CAGCAUCU | 20569 |
| 3520 | AAUGGAUG G CCCCAGAA | 8975 | UUCUGGGG GCCGAAAGGCGAGUGAGGUCU CAUCCAUU | 20570 |
| 3544 | UUGACAGA G UGUACACA | 8979 | UGUGUACA GCCGAAAGGCGAGUGAGGUCU UCUGUCAA | 20571 |
| 3546 | GACAGAGU G UACACAAU | 8980 | AUUGUGUA GCCGAAAGGCGAGUGAGGUCU ACUCUGUC | 20572 |
| 3560 | AAUCCAGA G UGACGUCU | 8982 | AGACGUCA GCCGAAAGGCGAGUGAGGUCU UCUGGAUU | 20573 |
| 3565 | AGAGUGAC G UCUGGUCU | 8984 | AGACCAGA GCCGAAAGGCGAGUGAGGUCU GUCACUCU | 20574 |
| 3570 | GACGUCUG G UCUUUUGG | 8985 | CCAAAACA GCCGAAAGGCGAGUGAGGUCU CAGACGUC | 20575 |
| 3578 | GUCUUUUG G UCUUUUGC | 8986 | GCAAAACA GCCGAAAGGCGAGUGAGGUCU CAAAAGAC | 20576 |
| 3580 | CUUUUGGU G UUUUGCUG | 8987 | CAGCAAAA GCCGAAAGGCGAGUGAGGUCU ACCAAAAG | 20577 |
| 3585 | GGUGUUUU G CUGUGGGA | 8988 | UCCCACAG GCCGAAAGGCGAGUGAGGUCU AAAACACC | 20578 |
| 3588 | CUUUUGCU G UGGGAAAU | 8989 | AUUUCCCA GCCGAAAGGCGAGUGAGGUCU AGCAAAAC | 20579 |
| 3608 | UUCCUUAG G UGCUUCUC | 8991 | GAGAAGCA GCCGAAAGGCGAGUGAGGUCU CUAAGGAA | 20580 |
| 3610 | CCUUAGGU G CUUCUCCA | 8992 | UGGAGAAG GCCGAAAGGCGAGUGAGGUCU ACCUAAGG | 20581 |
| 3628 | AUCCUGGG G UAAAGAUU | 8993 | AAUCUUUA GCCGAAAGGCGAGUGAGGUCU CCCAGGAU | 20582 |
| 3650 | AGAAUUUU G UAGGCGAU | 8997 | AUCGCCUA GCCGAAAGGCGAGUGAGGUCU AAAAUUCU | 20583 |
| 3654 | UUUUGUAG G CGAUUGAA | 8998 | UUCAAUCG GCCGAAAGGCGAGUGAGGUCU CUACAAAA | 20584 |
| 3682 | GAAUGAGG G CCCCUGAU | 9002 | AUCAGGGG GCCGAAAGGCGAGUGAGGUCU CCUCAUUC | 20585 |
| 3708 | CCAGAAAU G UACCAGAC | 9005 | GUCUGGUA GCCGAAAGGCGAGUGAGGUCU AUUUCUGG | 20586 |
| 3720 | CAGACCAU G CUGGACUG | 9007 | CAGUCCAG GCCGAAAGGCGAGUGAGGUCU AUGGUCUG | 20587 |
| 3728 | GCUGGACU G CUGGCACG | 9008 | CGUGCCAG GCCGAAAGGCGAGUGAGGUCU AGUCCAGC | 20588 |
| 3732 | GACUGCUC G CACGGGGA | 9009 | UCCCCGUG GCCGAAAGGCGAGUGAGGUCU CAGCAGUC | 20589 |
| 3741 | CACGGGGA G CCCAGUCA | 9010 | UCACUGGG GCCGAAAGGCGAGUGAGGUCU UCCCCGUG | 20590 |
| 3746 | GGAGCCCA G UCAGAGAC | 9011 | GUCUCUGA GCCGAAAGGCGAGUGAGGUCU UGGGCUCC | 20591 |
| 3759 | AGACCCAC G UUUUCACA | 9013 | UCUGAAAA GCCGAAAGGCGAGUGAGGUCU GUGGGUCU | 20592 |
| 3768 | UUUUCAGA G UUGGUGGA | 9014 | UCCACCAA GCCGAAAGGCGAGUGAGGUCU UCUGAAAA | 20593 |
| 3772 | CAGAGUUG G UGGAACAU | 9015 | AUGUUCCA GCCGAAAGGCGAGUGAGGUCU CAACUCUG | 20594 |
| 3795 | AAUCUCUU G CAAGCUAA | 9018 | UUAGCUUC GCCGAAAGGCGAGUGAGGUCU AAGAGAUU | 20595 |
| 3799 | UCUUGCAA G CUAAUGCU | 9019 | AGCAUUAG GCCGAAAGGCGAGUGAGGUCU UUGCAAGA | 20596 |
| 3805 | AAGCUAAU G CUCAGCAG | 9021 | CUGCUGAG GCCGAAAGGCGAGUGAGGUCU AUUAGCUU | 20597 |
| 3810 | AAUGCUCA G CAGGAUGG | 9022 | CCAUCCUG GCCGAAAGGCGAGUGAGGUCU UGAGCAUU | 20598 |
| 3818 | GCACGAUG G CAAAGACU | 9024 | AGUCUUUG GCCGAAAGGCGAGUGAGGUCU CAUCCUGC | 20599 |
| 3832 | ACUACAUU G UUCUUCCG | 9026 | CGGAAGAA GCCGAAAGGCGAGUGAGGUCU AAUGUAGU | 20600 |
| 3857 | GACUUUGA G CAUGGAAG | 9029 | CUUCCAUG GCCGAAAGGCGAGUGAGGUCU UCAAAGUC | 20601 |
| 3885 | CUCUCUCU G CCUACCUC | 9032 | GAGGUAGG GCCGAAAGGCGAGUGAGGUCU AGAGAGAG | 20602 |
| 3898 | CCUCACCU G UUUCCUGU | 9033 | ACAGGAAA GCCGAAAGGCGAGUGAGGUCU AGGUGAGG | 20603 |
| 3905 | UGUUUCCU G UAUGGAGG | 9034 | CCUCCAUA GCCGAAAGGCGAGUGAGGUCU AGGAAACA | 20604 |
| 3922 | AGGAGGAA G UAUGUGAC | 9035 | GUCACAUA GCCGAAAGGCGAGUGAGGUCU UUCCUCCU | 20605 |
| 3926 | GGAAGUAU G UGACCCCA | 9036 | UGGGGUCA GCCGAAAGGCGAGUGAGGUCU AUACUUCC | 20606 |
| 3955 | ACAACACA G CAGGAAUC | 9041 | GAUUCCUG GCCGAAAGGCGAGUGAGGUCU UGUGUUGU | 20607 |
| 3965 | AGGAAUCA G UCAGUAUC | 9043 | GAUACUGA GCCGAAAGGCGAGUGAGGUCU UGAUUCCU | 20608 |
| 3969 | AUCAGUCA G UAUCUGCA | 9044 | UGCAGAUA GCCGAAAGGCGAGUGAGGUCU UGACUGAU | 20609 |
| 3975 | CAGUAUCU G CAGAACAG | 9045 | CUGUUCUG GCCGAAAGGCGAGUGAGGUCU AGAUACUG | 20610 |
| 3983 | GCAGAACA G UAAGCGAA | 9047 | UUCGCUUA GCCGAAAGGCGAGUGAGGUCU UGUUCUGC | 20611 |
| 3987 | AACAGUAA G CGAAAGAG | 9048 | CUCUUUCG GCCGAAAGGCGAGUGAGGUCU UUACUGUU | 20612 |
| 3995 | GCGAAAGA G CCGGCCUG | 9049 | CAGGCCGG GCCGAAAGGCGAGUGAGGUCU UCUUUCGC | 20613 |
| 3999 | AAGAGCCG G CCUGUGAG | 9050 | CUCACAGG GCCGAAAGGCGAGUGAGGUCU CGGCUCUU | 20614 |
| 4003 | GCCGGCCU G UGAGUGUA | 9051 | UACACUCA GCCGAAAGGCGAGUGAGGUCU AGGCCGGC | 20615 |
| 4007 | GCCUGUGA G UGUAAAAA | 9052 | UUUUUACA GCCGAAAGGCGAGUGAGGUCU UCACAGGC | 20616 |
| 4009 | CUGUGAGU G UAAAAACA | 9053 | UGUUUUUA GCCGAAAGGCGAGUGAGGUCU ACUCACAG | 20617 |
| 4032 | GAUAUCCC G UUAGAAGA | 9056 | UCUUCUAA GCCGAAAGGCGAGUGAGGUCU GGGAUAUC | 20618 |
| 4048 | AACCAGAA G UAAAAGUA | 9058 | UACUUUUA GCCGAAAGGCGAGUGAGGUCU UUCUGGUU | 20619 |
| 4054 | AAGUAAAA G UAAUCCCA | 9059 | UGGGAUUA GCCGAAAGGCGAGUGAGGUCU UUUUACUU | 20620 |
| 4082 | GACGGACA G UGGUAUGG | 9066 | CCAUACCA GCCGAAAGGCGAGUGAGGUCU UGUCCGUC | 20621 |
| 4085 | GGACAGUG G UAUGGUUC | 9067 | GAACCAUA GCCGAAAGGCGAGUGAGGUCU CACUGUCC | 20622 |
| 4090 | GUGGUAUG G UUCUUGCC | 9068 | GGCAAGAA GCCGAAAGGCGAGUGAGGUCU CAUACCAC | 20623 |
| 4096 | UGGUUCUU G CCUCAGAA | 9069 | UUCUGAGG GCCGAAAGGCGAGUGAGGUCU AAGAACCA | 20624 |
| 4107 | UCAGAAGA G CUGAAAAC | 9070 | GUUUUCAG GCCGAAAGGCGAGUGAGGUCU UCUUCUGA | 20625 |
| 4151 | AUCUUUUG G UGGAAUGG | 9075 | CCAUUCCA GCCGAAAGGCGAGUGAGGUCU CAAAAGAU | 20626 |
| 4159 | GUGGAAUG G UGCCCAGC | 9077 | GCUGGGCA GCCGAAAGGCGAGUGAGGUCU CAUUCCAC | 20627 |
| 4161 | GGAAUGGU G CCCAGCAA | 9078 | UUGCUGGG GCCGAAAGGCGAGUGAGGUCU ACCAUUCC | 20628 |
| 4166 | GGUGCCCA G CAAAAGCA | 9079 | UGCUUUUG GCCGAAAGGCGAGUGAGGUCU UGGGCACC | 20629 |
| 4172 | CAGCAAAA G CAGGGAGU | 9080 | ACUCCCUG GCCGAAAGGCGAGUGAGGUCU UUUUGCUG | 20630 |
| 4179 | AGCAGGGA G UCUGUGGC | 9081 | GCCACAGA GCCGAAAGGCGAGUGAGGUCU UCCCUGCU | 20631 |
| 4183 | GGGAGUCU G UGGCAUCU | 9082 | AGAUGCCA GCCGAAAGGCGAGUGAGGUCU AGACUCCC | 20632 |
| 4186 | AGUCUGUG G CAUCUGAA | 9083 | UUCAGAUG GCCGAAAGGCGAGUGAGGUCU CACAGACU | 20633 |
| 4196 | AUCUGAAG G CUCAAACC | 9084 | GGUUUGAG GCCGAAAGGCGAGUGAGGUCU CUUCAGAU | 20634 |
| 4211 | CCAGACAA G CGGCUACC | 9087 | GGUAGCCG GCCGAAAGGCGAGUGAGGUCU UUCUCUGG | 20635 |
| 4214 | GACAAGCG G CUACCAGU | 9088 | ACUGGUAG GCCGAAAGGCGAGUGAGGUCU CGCUUGUC | 20636 |
| 4221 | GCCUACCA G UCCGGAUA | 9089 | UAUCCGGA GCCGAAAGGCGAGUGAGGUCU UGGUAGCC | 20637 |
| 4255 | ACACCACC G UGUACUCC | 9094 | GGAGUACA GCCGAAAGGCGAGUGAGGUCU GGUGGUGU | 20638 |
| 4257 | ACCACCGU G UACUCCAG | 9095 | CUGGAGUA GCCGAAAGGCGAGUGAGGUCU ACGGUGGU | 20639 |
| 4265 | GUACUCCA G UGAGGAAG | 9096 | CUUCCUCA GCCGAAAGGCGAGUGAGGUCU UGGAGUAC | 20640 |
| 4273 | GUGAGGAA G CAGAACUU | 9097 | AAGUUCUG GCCGAAAGGCGAGUGAGGUCU UUCCUCAC | 20641 |
| 4287 | CUUUUAAA G CUGAUAGA | 9099 | UCUAUCAG GCCGAAAGGCGAGUGAGGUCU UUUAAAAG | 20642 |
| 4303 | AGAUUGGA G UGCAAACC | 9102 | GGUUUGCA GCCGAAAGGCGAGUGAGGUCU UCCAAUCU | 20643 |
| 4305 | AUUGGAGU G CAAACCGG | 9103 | CCGGUUUG GCCGAAAGGCGAGUGAGGUCU ACUCCAAU | 20644 |
| 4313 | GCAAACCG G UAGCACAG | 9105 | CUGUGCUA GCCGAAAGGCGAGUGAGGUCU CGGUUUGC | 20645 |
| 4316 | AACCGGUA G CACAGCCC | 9106 | GGGCUGUG GCCGAAAGGCGAGUGAGGUCU UACCGGUU | 20646 |
| 4321 | GUAGCACA G CCCAGAUU | 9107 | AAUCUGGG GCCGAAAGGCGAGUGAGGUCU UGUGCUAC | 20647 |
| 4335 | AUUCUCCA G CCUGACUC | 9109 | GAGUCAGG GCCGAAAGGCGAGUGAGGUCU UGGAGAAU | 20648 |
| 4358 | CACACUGA G CUCUCCUC | 9112 | GAGGAGAG GCCGAAAGGCGAGUGAGGUCU UCAGUGUG | 20649 |
| 4369 | CUCCUCCU G UUUAAAAG | 9113 | CUUUUAAA GCCGAAAGGCGAGUGAGGUCU AGGAGGAG | 20650 |
| 4381 | AAAAGGAA G CAUCCACA | 9114 | UGUGGAUG GCCGAAAGGCGAGUGAGGUCU UUCCUUUU | 20651 |
| 4417 | CAUGAGAG G UCUGCUCA | 9117 | UGAGCAGA GCCGAAAGGCGAGUGAGGUCU CUCUCAUG | 20652 |
| 4421 | AGAGGUCU G CUCAGAUU | 9118 | AAUCUGAG GCCGAAAGGCGAGUGAGGUCU AGACCUCU | 20653 |
| 4435 | AUUUUGAA G UGUUGUUC | 9120 | GAACAACA GCCGAAAGGCGAGUGAGGUCU UUCAAAAU | 20654 |
| 4437 | UUUGAAGU G UUGUUCUU | 9121 | AAGAACAA GCCGAAAGGCGAGUGAGGUCU ACUUCAAA | 20655 |
| 4440 | GAAGUGUU G UUCUUUCC | 9122 | GGAAAGAA GCCGAAAGGCGAGUGAGGUCU AACACUUC | 20656 |
| 4453 | UUCCACCA G CAGGAAGU | 9123 | ACUUCCUG GCCGAAAGGCGAGUGAGGUCU UGGUGGAA | 20657 |
| 4460 | AGCAGGAA G UAGCCGCA | 9124 | UGCGGCUA GCCGAAAGGCGAGUGAGGUCU UUCCUGCU | 20658 |
| 4463 | AGGAAGUA G CCGCAUUU | 9125 | AAAUGCGG GCCGAAAGGCGAGUGAGGUCU UACUUCCU | 20659 |
| 4466 | AAGUAGCC G CAUUUGAU | 9126 | AUCAAAUG GCCGAAAGGCGAGUGAGGUCU GGCUACUU | 20660 |
| 4509 | CUCGGACU G CAGGGAGC | 9132 | GCUCCCUG GCCGAAAGGCGAGUGAGGUCU AGUCCGAG | 20661 |
| 4516 | UGCAGGGA G CCAGUCUU | 9133 | AAGACUGG GCCGAAAGGCGAGUGAGGUCU UCCCUGCA | 20662 |
| 4520 | GGGAGCCA G UCUUCUAG | 9134 | CUAGAAGA GCCGAAAGGCGAGUGAGGUCU UGGCUCCC | 20663 |
| 4529 | UCUUCUAC C CAUAUCCU | 9135 | ACGAUAUC GCCGAAAGGCGAGUGAGGUCU CUACAACA | 20664 |
| 4545 | UGGAAGAG G CUUGUGAC | 9138 | GUCACAAG GCCGAAAGGCGAGUGAGGUCU CUCUUCCA | 20665 |
| 4549 | AGAGGCUU G UGACCCAA | 9139 | UUGGGUCA GCCGAAAGGCGAGUGAGGUCU AAGCCUCU | 20666 |
| 4562 | CCAAGAAU G UGUCUGUG | 9142 | CACAGACA GCCGAAAGGCGAGUGAGGUCU AUUCUUGG | 20667 |
| 4564 | AAGAAUGU G UCUGUGUC | 9143 | GACACAGA GCCGAAAGGCGAGUGAGGUCU ACAUUCUU | 20668 |
| 4568 | AUGUGUCU G UGUCUUCU | 9144 | AGAAGACA GCCGAAAGGCGAGUGAGGUCU AGACACAU | 20669 |
| 4570 | GUGUCUGU G UCUUCUCC | 9145 | GGAGAAGA GCCGAAAGGCGAGUGAGGUCU ACAGACAC | 20670 |
| 4581 | UUCUCCCA G UGUUGACC | 9146 | GGUCAACA GCCGAAAGGCGAGUGAGGUCU UGGGAGAA | 20671 |
| 4583 | CUCCCAGU G UUGACCUG | 9147 | CAGGUCAA GCCGAAAGGCGAGUGAGGUCU ACUGGGAG | 20672 |
| 4618 | UUUAAAAA G CAUUAUCA | 9152 | UGAUAAUG GCCGAAAGGCGAGUGAGGUCU UUUUUAAA | 20673 |
| 4628 | AUUAUCAU G CCCCUGCU | 9156 | AGCAGGGG GCCGAAAGGCGAGUGAGGUCU AUGAUAAU | 20674 |
| 4634 | AUGCCCCU G CUGCGGGU | 9157 | ACCCGCAG GCCGAAAGGCGAGUGAGGUCU AGGGGCAU | 20675 |
| 4637 | CCCCUGCU G CGGGUCUC | 9158 | GAGACCCG GCCGAAAGGCGAGUGAGGUCU AGCAGGGG | 20676 |
| 4641 | UGCUGCGG G UCUCACCA | 9159 | UGGUGAGA GCCGAAAGGCGAGUGAGGUCU CCGCAGCA | 20677 |
| 4653 | CACCAUGG G UUUAGAAC | 9162 | GUUCUAAA GCCGAAAGGCGAGUGAGGUCU CCAUGGUG | 20678 |
| 4667 | AACAAAGA G CUUCAAGC | 9164 | GCUUGAAG GCCGAAAGGCGAGUGAGGUCU UCUUUGUU | 20679 |
| 4674 | AGCUUCAA G CAAUGGCC | 9165 | GGCCAUUG GCCGAAAGGCGAGUGAGGUCU UUGAAGCU | 20680 |
| 4680 | AAGCAAUG G CCCCAUCC | 9167 | GGAUGGGG GCCGAAAGGCGAGUGAGGUCU CAUUGCUU | 20681 |
| 4697 | UCAAAGAA G UAGCAGUA | 9169 | UACUGCUA GCCGAAAGGCGAGUGAGGUCU UUCUUUGA | 20682 |
| 4700 | AAGAAGUA G CAGUACCU | 9170 | AGGUACUG GCCGAAAGGCGAGUGAGGUCU UACUUCUU | 20683 |
| 4703 | AAGUAGCA G UACCUGGG | 9171 | CCCAGGUA GCCGAAAGGCGAGUGAGGUCU UGCUACUU | 20684 |
| 4714 | CCUGGGGA G CUGACACU | 9173 | AGUGUCAG GCCGAAAGGCGAGUGAGGUCU UCCCCAGG | 20685 |
| 4726 | ACACUUCU G UAAAACUA | 9176 | UAGUUUUA GCCGAAAGGCGAGUGAGGUCU AGAAGUGU | 20686 |
| 4748 | UAAACCAG G CAACGUAA | 9180 | UUACGUUG GCCGAAAGGCGAGUGAGGUCU CUGGUUUA | 20687 |
| 4753 | CAGGCAAC G UAAGUGUU | 9182 | AACACUUA GCCGAAAGGCGAGUGAGGUCU GUUGCCUG | 20688 |
| 4757 | CAACGUAA G UGUUCCAG | 9183 | CUCGAACA GCCGAAAGGCGAGUGAGGUCU UUACGUUG | 20689 |
| 4759 | ACGUAAGU G UUCGAGGU | 9184 | ACCUCGAA GCCGAAAGGCGAGUGAGGUCU ACUUACGU | 20690 |
| 4766 | UGUUCGAG G UGUUGAAG | 9185 | CUUCAACA GCCGAAAGGCGAGUGAGGUCU CUCGAACA | 20691 |
| 4768 | UUCGAGGU G UUGAAGAU | 9186 | AUCUUCAA GCCGAAAGGCGAGUGAGGUCU ACCUCGAA | 20692 |
| 4788 | AAGGAUUU G CAGGGCUG | 9189 | CAGCCCUG GCCGAAAGGCGAGUGAGGUCU AAAUCCUU | 20693 |
| 4793 | UUUGCAGG G CUGAGUCU | 9190 | AGACUCAG GCCGAAAGGCGAGUGAGGUCU CCUGCAAA | 20694 |
| 4798 | AGGGCUGA G UCUAUCCA | 9191 | UGGAUAGA GCCGAAAGGCGAGUGAGGUCU UCAGCCCU | 20695 |
| 4811 | UCCAAGAG G CUUUGUUU | 9193 | AAACAAAG GCCGAAAGGCGAGUGAGGUCU CUCUUGGA | 20696 |
| 4816 | GAGGCUUU G UUUAGGAC | 9194 | GUCCUAAA GCCGAAAGGCGAGUGAGGUCU AAAGCCUC | 20697 |
| 4825 | UUUAGGAC G UGGGUCCC | 9196 | GGGACCCA GCCGAAAGGCGAGUGAGGUCU GUCCUAAA | 20698 |
| 4829 | GGACGUGG G UCCCAAGC | 9197 | GCUUGGGA GCCGAAAGGCGAGUGAGGUCU CCACGUCC | 20699 |
| 4836 | GGUCCCAA G CCAAGCCU | 9198 | ACGGUUGG GCCGAAAGGCGAGUGAGGUCU UUGGGACC | 20700 |
| 4841 | CAAGCCAA G CCUUAAGU | 9199 | ACUUAAGG GCCGAAAGGCGAGUGAGGUCU UUGGCUUG | 20701 |
| 4848 | AGCCUUAA G UGUGGAAU | 9200 | AUUCCACA GCCGAAAGGCGAGUGAGGUCU UUAAGGCU | 20702 |
| 4850 | CCUUAAGU G UGGAAUUC | 9201 | GAAUUCCA GCCGAAAGGCGAGUGAGGUCU ACUUAAGG | 20703 |
| 4883 | AGACUAAC G UUACCUUG | 9207 | CAAGGUAA GCCGAAAGGCGAGUGAGGUCU GUUAGUCU | 20704 |
| 4891 | CUUACCUU G CUUUCCAG | 9209 | CUCCAAAG GCCGAAAGGCGAGUGAGGUCU AAGGUAAC | 20705 |
| 4901 | UUUGGAGA G UACUGGAG | 9210 | CUCCAGUA GCCGAAAGGCGAGUGAGGUCU UCUCCAAA | 20706 |
| 4909 | GUACUGGA G CCUGCAAA | 9212 | UUUGCAGG GCCGAAAGGCGAGUGAGGUCU UCCAGUAC | 20707 |
| 4913 | UGGAGCCU G CAAAUGCA | 9213 | UGCAUUUC GCCGAAAGGCGAGUGAGGUCU AGGCUCCA | 20708 |
| 4919 | CUGCAAAU G CAUUGUGU | 9215 | ACACAAUG GCCGAAAGGCGAGUGAGGUCU AUUUGCAG | 20709 |
| 4924 | AAUGCAUU G UGUUUGCU | 9217 | AGCAAACA GCCGAAAGGCGAGUGAGGUCU AAUGCAUU | 20710 |
| 4926 | UGCAUUGU G UUUGCUCU | 9218 | AGAGCAAA GCCGAAAGGCGAGUGAGGUCU ACAAUGCA | 20711 |
| 4930 | UUGUGUUU G CUCUGGUG | 9219 | CACCAGAG GCCGAAAGGCGAGUGAGGUCU AAACACAA | 20712 |
| 4936 | UUGCUCUG G UGGAGGUG | 9220 | CACCUCCA GCCGAAAGGCGAGUGAGGUCU CAGAGCAA | 20713 |
| 4942 | UGGUGGAG G UGGGCAUG | 9221 | CAUGCCCA GCCGAAAGGCGAGUGAGGUCU CUCCACCA | 20714 |
| 4946 | GGAGGUGG G CAUGGGGU | 9222 | ACCCCAUG GCCGAAAGGCGAGUGAGGUCU CCACCUCC | 20715 |
| 4953 | GGCAUGGG G UCUGUUCU | 9224 | AGAACAGA GCCGAAAGGCGAGUGAGGUCU CCCAUGCC | 20716 |
| 4957 | UGGGGUCU G UUCUGAAA | 9225 | UUUCAGAA GCCGAAAGGCGAGUGAGGUCU AGACCCCA | 20717 |
| 4967 | UCUGAAAU G UAAAGGGU | 9227 | ACCCUUUA GCCGAAAGGCGAGUGAGGUCU AUUUCAGA | 20718 |
| 4974 | UGUAAAGG G UUCAGACG | 9228 | CGUCUGAA GCCGAAAGGCGAGUGAGGUCU CCUUUACA | 20719 |
| 4985 | CAGACGGG G UUUCUGGU | 9230 | ACCAGAAA GCCGAAAGGCGAGUGAGGUCU CCCGUCUG | 20720 |
| 4992 | GGUUUCUG G UUUUAGAA | 9231 | UUCUAAAA GCCGAAAGGCGAGUGAGGUCU CAGAAACC | 20721 |
| 5002 | UUUAGAAG G UUGCGUGU | 9232 | ACACGCAA GCCGAAAGGCGAGUGAGGUCU CUUCUAAA | 20722 |
| 5005 | AGAAGGUU G CGUGUUCU | 9233 | AGAACACG GCCGAAAGGCGAGUGAGGUCU AACCUUCU | 20723 |
| 5007 | AAGGUUGC G UGUUCUUC | 9234 | GAAGAACA GCCGAAAGGCGAGUGAGGUCU GCAACCUU | 20724 |
| 5009 | GGUUGCGU G UUCUUCGA | 9235 | UCGAAGAA GCCGAAAGGCGAGUGAGGUCU ACGCAACC | 20725 |
| 5018 | UUCUUCGA G UUGCCCUA | 9236 | UAGCCCAA GCCGAAAGGCGAGUGAGGUCU UCGAAGAA | 20726 |
| 5023 | CGAGUUGG G CUAAAGUA | 9237 | UACUUUAG GCCGAAAGGCGAGUGAGGUCU CCAACUCG | 20727 |
| 5029 | GGGCUAAA G UAGAGUUC | 9238 | GAACUCUA GCCGAAAGGCGAGUGAGGUCU UUUAGCCC | 20728 |
| 5034 | AAAGUAGA G UUCGUUGU | 9239 | ACAACGAA GCCGAAAGGCGAGUGAGGUCU UCUACUUU | 20729 |
| 5038 | UAGAGUUC G UUGUGCUG | 9240 | CAGCACAA GCCGAAAGGCGAGUGAGGUCU GAACUCUA | 20730 |
| 5041 | AGUUCGUU G UGCUGUUU | 9241 | AAACAGCA GCCGAAAGGCGAGUGAGGUCU AACGAACU | 20731 |
| 5043 | UUCGUUGU G CUGUUUCU | 9242 | AGAAACAG GCCGAAAGGCGAGUGAGGUCU ACAACGAA | 20732 |
| 5046 | GUUGUGCU G UUUCUGAC | 9243 | GUCAGAAA GCCGAAAGGCGAGUGAGGUCU AGCACAAC | 20733 |
| 5066 | UAAUGAGA G UUCCUUCC | 9246 | GGAAGGAA GCCGAAAGGCGAGUGAGGUCU UCUCAUUA | 20734 |
| 5080 | UCCAGACC G UUAGCUGU | 9248 | ACAGCUAA GCCGAAAGGCGAGUGAGGUCU GGUCUGGA | 20735 |
| 5084 | GACCGUUA G CUGUCUCC | 9249 | GGAGACAG GCCGAAAGGCGAGUGAGGUCU UAACGGUC | 20736 |
| 5087 | CGUUAGCU G UCUCCUUG | 9250 | CAAGGAGA GCCGAAAGGCGAGUGAGGUCU AGCUAACG | 20737 |
| 5095 | GUCUCCUG G CCAAGCCC | 9251 | GGGCUUGG GCCGAAAGGCGAGUGAGGUCU AAGGAGAC | 20738 |
| 5100 | CUUGCCAA G CCCCAGGA | 9252 | UCCUGGGG GCCGAAAGGCGAGUGAGGUCU UUGGCAAG | 20739 |
| 5119 | AAAAUGAU G CAGCUCUG | 9255 | CAGAGCUG GCCGAAAGGCGAGUGAGGUCU AUCAUUUU | 20740 |
| 5122 | AUGAUGCA G CUCUGGCU | 9256 | AGCCAGAG GCCGAAAGGCGAGUGAGGUCU UGCAUCAU | 20741 |
| 5128 | CAGCUCUG G CUCCUUGU | 9257 | ACAAGGAG GCCGAAAGGCGAGUGAGGUCU CAGAGCUG | 20742 |
| 5135 | GGCUCCUU G UCUCCCAG | 9258 | CUGGGAGA GCCGAAAGGCGAGUGAGGUCU AAGGAGCC | 20743 |
| 5144 | UCUCCCAG G CUGAUCCU | 9259 | AGGAUCAG GCCGAAAGGCGAGUGAGGUCU CUGGGAGA | 20744 |
| 5185 | GACAUUCA G CUCAAGGC | 9267 | GCCUUGAG GCCGAAAGGCGAGUGAGGUCU UGAAUGUC | 20745 |
| 5192 | AGCUCAAG G CUCCCUGC | 9268 | GCAGGGAG GCCGAAAGGCGAGUGAGGUCU CUUGAGCU | 20746 |
| 5199 | GGCUCCCU G CCGUGUUG | 9269 | CAACACGG GCCGAAAGGCGAGUGAGGUCU AGGGAGCC | 20747 |
| 5202 | UCCCUGCC G UGUUGAAG | 9270 | CUUCAACA GCCGAAAGGCGAGUGAGGUCU GGCAGGGA | 20748 |
| 5204 | CCUGCCGU G UUGAAGAG | 9271 | CUCUUCAA GCCGAAAGGCGAGUGAGGUCU ACGGCAGG | 20749 |
| 5212 | GUUGAAGA G UUCUGACU | 9272 | AGUCAGAA GCCGAAAGGCGAGUGAGGUCU UCUUCAAC | 20750 |
| 5221 | UUCUGACU G CACAAACC | 9274 | GGUUUGUG GCCGAAAGGCGAGUGAGGUCU AGUCAGAA | 20751 |
| 5231 | ACAAACCA G CUUCUGGU | 9277 | ACCAGAAG GCCGAAAGGCGAGUGAGGUCU UGGUUUGU | 20752 |
| 5238 | AGCUUCUG G UUUCUUCU | 9278 | AGAAGAAA GCCGAAAGGCGAGUGAGGUCU CAGAAGCU | 20753 |
| 5268 | UCAUAUCU G UCCUGAUG | 9284 | CAUCAGGA GCCGAAAGGCGAGUGAGGUCU AGAUAUGA | 20754 |
| 5276 | GUCCUGAU G UGAUAUGU | 9286 | ACAGAUCA GCCGAAAGGCGAGUGAGGUCU AUCAGGAC | 20755 |
| 5283 | UGUGAUAU G UCUGAGAC | 9289 | GUCUCAGA GCCGAAAGGCGAGUGAGGUCU AUAUCACA | 20756 |
| 5297 | GACUCAAU G CGGGAGGU | 9292 | ACCUCCCG GCCGAAAGGCGAGUGAGGUCU AGUCAGUC | 20757 |
| 5304 | UGCGGGAG G UUCAAUGU | 9293 | ACAUUGAA GCCGAAAGGCGAGUGAGGUCU CUCCCGCA | 20758 |
| 5311 | GGUUCAAU G UGAAGCUG | 9295 | CAGCUUCA GCCGAAAGGCGAGUGAGGUCU AUUGAACC | 20759 |
| 5316 | AAUGUGAA G CUGUGUGU | 9296 | ACACACAG GCCGAAAGGCGAGUGAGGUCU UUCACAUU | 20760 |
| 5319 | GUGAAGCU G UGUGUGGU | 9297 | ACCACACA GCCGAAAGGCGAGUGAGGUCU AGCUUCAC | 20761 |
| 5321 | GAAGCUGU G UGUGGUGU | 9298 | ACACCACA GCCGAAAGGCGAGUGAGGUCU ACAGCUUC | 20762 |
| 5323 | AGCUGUGU G UGGUGUCA | 9299 | UGACACCA GCCGAAAGGCGAGUGAGGUCU ACACAGCU | 20763 |
| 5326 | UGUGUGUG G UGUCAAAG | 9300 | CUUUGACA GCCGAAAGGCGAGUGAGGUCU CACACACA | 20764 |
| 5328 | UGUGUGGU G UCAAAGUU | 9301 | AACUUUGA GCCGAAAGGCGAGUGAGGUCU ACCACACA | 20765 |
| 5334 | GUGUCAAA G UUUCAGGA | 9302 | UCCUGAAA GCCGAAAGGCGAGUGAGGUCU UUUGACAC | 20766 |
| 5359 | ACCCUUUU G UUCUUCCC | 9305 | GGGAAGAA GCCGAAAGGCGAGUGAGGUCU AAAAGGGU | 20767 |
| 5371 | UUCCCCCU G UCCCCAAC | 9306 | GUUGGGGA GCCGAAAGGCGAGUGAGGUCU AGGGGGAA | 20768 |
| 5393 | CUCACCCC G CAACCCAU | 9310 | AUGGGUUG GCCGAAAGGCGAGUGAGGUCU GGGGUGAG | 20769 |
| 5404 | ACCCAUCA G UAUUUUAG | 9313 | CUAAAAUA GCCGAAAGGCGAGUGAGGUCU UGAUGGGU | 20770 |
| 5412 | GUAUUUUA G UUAUUUGG | 9315 | CCAAAUAA GCCGAAAGGCGAGUGAGGUCU UAAAAUAC | 20771 |
| 5420 | GUUAUUUG G CCUCUACU | 9317 | AGUAGAGG GCCGAAAGGCGAGUGAGGUCU CAAAUAAC | 20772 |
| 5432 | CUACUCCA G UAAACCUG | 9319 | CAGGUUUA GCCGAAAGGCGAGUGAGGUCU UGGAGUAG | 20773 |
| 5446 | CUGAUUUG G UUUGUUCA | 9322 | UGAACAAA GCCGAAAGGCGAGUGAGGUCU CCAAUCAG | 20774 |
| 5450 | UUGGGUUU G UUCACUCU | 9323 | AGAGUGAA GCCGAAAGGCGAGUGAGGUCU AAACCCAA | 20775 |
| 5473 | GAUUAUUA G CCAGACUU | 9328 | AAGUCUGG GCCGAAAGGCGAGUGAGGUCU UAAUAAUC | 20776 |
| 5497 | AUUUUAUA G CCCAAAUU | 9333 | AAUUUGGG GCCGAAAGGCGAGUGAGGUCU UAUAAAAU | 20777 |
| 5518 | CAUCUAUU G UAUUAUUU | 9339 | AAAUAAUA GCCGAAAGGCGAGUGAGGUCU AAUAGAUG | 20778 |
| 5545 | CAUAUAGA G CUAUUUCU | 9346 | AGAAAUAG GCCGAAAGGCGAGUGAGGUCU UCUAUAUG | 20779 |
| 5564 | UGAUUUUU G CCCUUGUU | 9350 | AACAAGGG GCCGAAAGGCGAGUGAGGUCU AAAAAUCA | 20780 |
| 5570 | UUGCCCUU G UUCUGUCC | 9351 | GGACAGAA GCCGAAAGGCGAGUGAGGUCU AAGGGCAA | 20781 |
| 5575 | CUUGUUCU G UCCUUUUU | 9352 | AAAAAGGA GCCGAAAGGCGAGUGAGGUCU AGAACAAG | 20782 |
| 5599 | AAGAAAAU G UGUUUUUU | 9354 | AAAAAACA GCCGAAAGGCGAGUGAGGUCU AUUUUCUU | 20783 |
| 5601 | GAAAAUGU G UUUUUUGU | 9355 | ACAAAAAA GCCGAAAGGCGAGUGAGGUCU ACAUUUUC | 20784 |
| 5608 | UGUUUUUU G UUUGGUAC | 9356 | GUACCAAA GCCGAAAGGCGAGUGAGGUCU AAAAAACA | 20785 |
| 5613 | UUUGUUUG G UACCAUAG | 9357 | CUAUGGUA GCCGAAAGGCGAGUGAGGUCU CAAACAAA | 20786 |
| 5621 | GUACCAUA G UGUGAAAU | 9360 | AUUUCACA GCCGAAAGGCGAGUGAGGUCU UAUGGUAC | 20787 |
| 5623 | ACCAUAGU G UGAAAUGC | 9361 | GCAUUUCA GCCGAAAGGCGAGUGAGGUCU ACUAUGGU | 20788 |
| 5630 | UGUGAAAU G CUGGGAAC | 9363 | GUUCCCAG GCCGAAAGGCGAGUGAGGUCU AUUUCACA | 20789 |
| 5655 | UAAGACAU G CUAUGGCA | 9370 | UGCCAUAG GCCGAAAGGCGAGUGAGGUCU AUGUCUUA | 20790 |
| 5661 | AUGCUAUG G CACAUAUA | 9372 | UAUAUGUG GCCGAAAGGCGAGUGAGGUCU CAUAGCAU | 20791 |
| 5676 | UAUUUAUA G UCUGUUUA | 9378 | UAAACAGA GCCGAAAGGCGAGUGAGGUCU UAUAAAUA | 20792 |
| 5680 | UAUAGUCU G UUUAUGUA | 9379 | UACAUAAA GCCGAAAGGCGAGUGAGGUCU AGACUAUA | 20793 |
| 5686 | CUGUUUAU G UAGAAACA | 9381 | UGUUUCUA GCCGAAAGGCGAGUGAGGUCU AUAAACAG | 20794 |
| 5698 | AAACAAAU G UAAUAUAU | 9384 | AUAUAUUA GCCGAAAGGCGAGUGAGGUCU AUUUGUUU | 20795 |
| 5711 | AUAUUAAA G CCUUAUAU | 9388 | AUAUAAGG GCCGAAAGGCGAGUGAGGUCU UUUAAUAU | 20796 |
| 5732 | UGAACUUU G UACUAUUC | 9394 | GAAUAGUA GCCGAAAGGCGAGUGAGGUCU AAAGUUCA | 20797 |
| 5748 | CACAUUUU G UAUCAGUA | 9399 | UACUGAUA GCCGAAAGGCGAGUGAGGUCU AAAAUGUG | 20798 |
| 5754 | UUGUAUCA G UAUUAUGU | 9401 | ACAUAAUA GCCGAAAGGCGAGUGAGGUCU UGAUACAA | 20799 |
| 5761 | AGUAUUAU G UAGCAUAA | 9404 | UUAUGCUA GCCGAAAGGCGAGUGAGGUCU AUAAUACU | 20800 |
| 5764 | AUUAUGUA G CAUAACAA | 9405 | UUGUUAUG GCCGAAAGGCGAGUGAGGUCU UACAUAAU | 20801 |
| 5775 | UAACAAAG G UCAUAAUG | 9408 | CAUUAUGA GCCGAAAGGCGAGUGAGGUCU CUUUGUUA | 20802 |
| 5783 | GUCAUAAU G CUUUCAGC | 9411 | GCUGAAAG GCCGAAAGGCGAGUGAGGUCU AUUAUGAC | 20803 |
| 5790 | UGCUUUCA G CAAUUGAU | 9412 | AUCAAUUG GCCGAAAGGCGAGUGAGGUCU UGAAAGCA | 20804 |
| 5799 | CAAUUGAU G UCAUUUUA | 9415 | UAAAAUGA GCCGAAAGGCGAGUGAGGUCU AUCAAUUG | 20805 |

Input Sequence = AF035121.
Cut Site = G/Y
Arm Length = 8.
Core Sequence = GCcgaaagGCGaGuCaaGGuCu
AF035121 (Homo sapiens KDR/flk-1 protein mRNA, complete cds.; Acc# AF035121; 5830 bp)

TABLE 24
Subcutaneous 4T1 Tumor Model: Spontaneous Lung Metastases
Spontaneous pulmonary metastases from subcutaneous 4T1 tumor enumerated at time of
sacrifice in control and ANGIOZYME-treated mice

| | | 4T1 Tumor | ANGIOZYME | Mice per | Mean No. |
|---|---|---|---|---|---|
| Experimental Group | | Dose | Dose | Group | Metastases (SD) |
| A | Experiment 1 Control | 5.0 × 10⁵ | — | 5 | 32.8 (23.7) |
| B | Low Dose | 5.0 × 10⁵ | 300 mg/m²/day | 5 | 7.2 (5.4)^{a} |
| | ANGIOZYME | | | | |
| C | Experiment 2 Control | 1.0 × 10⁶ | — | 4 | 68.5 (9.1) |
| D | High Dose | 1.0 × 10⁶ | 600 mg/m²/day | 4 | 11.3 (2.5)^{b} |
| | ANGIOZYME | | | | |

^{a}Statistically significant difference compared to control group A (p-value <0.05).
^{b}Statistically significant difference compared to control group C (p-value <0.01).

1. A method for down regulating expression of vascular endothelial growth factor receptors (VEGFRs) in a cell comprising contacting the cell with a nucleic acid molecule that down regulates expression of VEGFR1 and a nucleic acid molecule that down regulates expression of VEGFR2 under conditions suitable for down regulating expression of VEGFRs in the cell.

2. The method of claim 1, wherein the nucleic acid molecule that down regulates expression of VEGFR1 and the nucleic acid molecule that down regulates expression of VEGFR2 are enzymatic nucleic acid molecules.

3. The method of claim 1, wherein the nucleic acid molecule that down regulates expression of VEGFR1 and the nucleic acid molecule that down regulates expression of VEGFR2 are short interfering nucleic acid molecules.

4. The method of claim 1, wherein the nucleic acid molecule that down regulates expression of VEGFR1 and the nucleic acid molecule that down regulates expression of VEGFR2 are antisense nucleic acid molecules.

5. The method of claim 1, wherein the nucleic acid molecule that down regulates expression of VEGFR1 or VEGFR2 is selected from the group consisting of an enzmatic nucleic acid molecule, an antisense nucleic acid molecule, and a short interfering nucleic acid molecule.

6. The method of claim 1, wherein said cell is a mammalian cell.

7. The method of claim 1, wherein said cell is a human cell.

8. The method of claim 1, wherein one or both of the nucleic acid molecules contact the cell in the presence of a delivery reagent.

9. The method of claim 8, wherein said delivery reagent is a lipid.

10. The method of claim 9, wherein said lipid is a cationic lipid.

11. The method of claim 8, wherein said delivery reagent is a liposome.

12. A method for treating cancer in a subject comprising administering to the subject a nucleic acid molecule that down regulates expression of VEGFR1 and a nucleic acid molecule that down regulates expression of VEGFR2 under conditions suitable for the treatment.

13. The method of claim 12, wherein the nucleic acid molecule that down regulates expression of VEGFR1 and the nucleic acid molecule that down regulates expression of VEGFR2 are enzymatic nucleic acid molecules.

14. The method of claim 12, wherein the nucleic acid molecule that down regulates expression of VEGFR1 and the nucleic acid molecule that down regulates expression of VEGFR2 are short interfering nucleic acid molecules.

15. The method of claim 12, wherein the nucleic acid molecule that down regulates expression of VEGFR1 and the nucleic acid molecule that down regulates expression of VEGFR2 are antisense nucleic acid molecules.

16. The method of claim 12, wherein the nucleic acid molecule that down regulates expression of VEGFR1 or VEGFR2 is selected from the group consisting of an enzmatic nucleic acid molecule, an antisense nucleic acid molecule, and a short interfering nucleic acid molecule.

17. The method of claim 12, wherein the cancer is selected from the group consisting of breast cancer, ovarian cancer, lung cancer, colorectal cancer, and melanoma.

18. The method of claim 12, further comprising the administration of one or more therapies under conditions suitable for the treatment.

19. The method of claim 12, wherein said administration is in the presence of a delivery reagent.

20. The method of claim 19, wherein said delivery reagent is a lipidcationic lipid, phospholipid, or liposome.

21. A method for treating cancer in a subject comprising administering to the subject a nucleic acid molecule that down regulates expression of a VEGF receptor in combination with interferon under conditions suitable for the treatment.

22. The method of claim 21, wherein the nucleic acid molecule that down regulates expression of a VEGF receptor is an enzymatic nucleic acid molecule.

23. The method of claim 21, wherein the nucleic acid molecule that down regulates expression of a VEGF receptor is an antisense acid molecule.

24. The method of claim 21, wherein the nucleic acid molecule that down regulates expression of a VEGF receptor is a short interfering nucleic acid molecule.

25. The method of claim 21, wherein the interferon is type I interferon.

26. The method of claim 25, wherein said type I interferon and the compound are administered simultaneously.

27. The method of claim 25, wherein said type I interferon and the compound are administered separately.

28. The method of claim 25, wherein said type I interferon is selected from the group consisiting of interferon alpha, interferon beta, consensus interferon, polyethylene glycol interferon, polyethylene glycol interferon alpha 2a, polyethylene glycol interferon alpha 2b, and polyethylene glycol consensus interferon.

29. The method of claim 21, wherein the cancer is selected from the group consisting of breast cancer, ovarian cancer, lung cancer, colorectal cancer, and melanoma.

30. The method of claim 21, wherein said administration is in the presence of a delivery reagent.

31. The method of claim 30, wherein said delivery reagent is a lipid, cationic lipid, phospholipid, or liposome.
